(19) United States
(12) Patent Application Publication
(10) Pub. No.: US 2024/0147846 A1
(43) Pub. Date: May 2, 2024

(54) ORGANIC ELECTROLUMINESCENT ELEMENT AND ELECTRONIC DEVICE

(71) Applicant: IDEMITSU KOSAN CO.,LTD., Chiyoda-ku (JP)

(72) Inventors: Shota TANAKA, Sodegaura-shi (JP); Shintaro BAN, Sodegaura-shi (JP); Satomi TASAKI, Sodegaura-shi (JP); Hiroaki ITOI, Sodegaura-shi (JP); Tasuku HAKETA, Sodegaura-shi (JP); Yuki NAKANO, Sodegaura-shi (JP)

(73) Assignee: IDEMITSU KOSAN CO.,LTD., Chiyoda-ku (JP)

(21) Appl. No.: 18/256,036

(22) PCT Filed: Dec. 8, 2021
(86) PCT No.: PCT/JP2021/045024

§ 371 Date: Jun. 5, 2023

(30) Foreign Application Priority Data
Dec. 9, 2020 (JP) 2020-204531

(51) Int. Cl. H10K 85/60; C07D 209/86; C07D 307/77; C09K 11/06

(52) U.S. Cl. CPC H10K 85/636; C07D 209/86; C07D 307/77; C09K 11/06; H10K 85/615; H10K 85/6574; H10K 50/12

(57) ABSTRACT

An organic electroluminescent element including, an anode, a cathode, and an organic layer disposed between the anode and the cathode and including a light emitting zone. The organic layer includes a first layer containing a first compound and a second layer containing a second compound. The first layer and the second layer are different layers. The first compound contains 1 mass % or more of a compound having one or more deuterium atoms in a compound represented by the following formula (1), and the second compound contains 1 mass % or more of a compound having one or more deuterium atoms in a compound represented by the following formula (2),

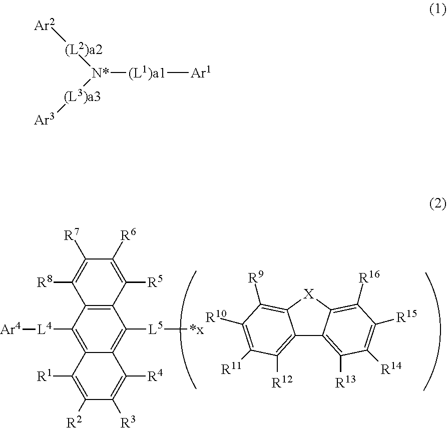

### TECHNICAL FIELD

The present invention relates to an organic electroluminescent element and an electronic device.

### BACKGROUND ART

In general, an organic electroluminescent element (an organic EL element) includes an anode, a cathode, and an organic layer sandwiched between the anode and the cathode. When a voltage is applied between the two electrodes, electrons are injected from a cathode side into a light emitting region, holes are injected from an anode side into the light emitting region, the injected electrons and holes are recombined in the light emitting region to generate an excited state, and light is emitted when the excited state returns to a ground state. Therefore, development of a compound that efficiently transports electrons or holes to a light emitting region and promotes recombination of the electrons and holes is important for obtaining a high-performance organic EL element. In recent years, with further spread of a smart phone, an organic EL television, an organic EL illumination, and the like using the organic EL element, there has been a demand for a compound that satisfies a requirement for a sufficient element lifetime.

For example, PTLs 1 to 5 disclose an aromatic amine compound to be used in an organic EL element and an anthracene compound to be used in the organic EL element.

### CITATION LIST

### Patent Literature

PTL 1: WO 2016/009823

PTL 2: WO 2020/111733

PTL 3: US 2020/111986 A1

PTL 4: US 2017/200899 A1

PTL 5: KR 10-2019-30731 A

### SUMMARY OF INVENTION

### Technical Problem

In the related art, many compounds have been reported as a material for producing an organic EL element, but a compound that further improves characteristics of the organic EL element is still required. As for the aromatic amine compound to be used in the organic EL element, a compound that further improves the characteristics of the organic EL element, particularly a compound that can achieve a much longer lifetime is also still required.

The present invention has been made to solve the above problems, and an object of the present invention is to provide a high-performance organic EL element, more specifically an organic EL element capable of achieving a longer lifetime, and an electronic device including the organic EL element.

### Solution to Problem

As a result of intensive studies to solve the above problems, the present inventors have found that when an organic layer disposed between an anode and a cathode includes a first layer containing a first compound and a second layer containing a second compound, the first compound contains a compound having one or more deuterium atoms in a compound represented by the following formula (1), and the second compound contains a compound having one or more deuterium atoms in a compound represented by the following formula (2), a high-performance organic EL element can be implemented, more specifically an organic EL element with a longer lifetime can be implemented, thereby completing the present invention.

In one aspect, the present invention provides an organic electroluminescent element including: an anode; a cathode facing the anode; and an organic layer disposed between the anode and the cathode and including a light emitting zone. The organic layer includes a first layer containing a first compound and a second layer containing a second compound. The first layer and the second layer are different layers. The first compound contains 1 mass % or more of a compound having one or more deuterium atoms in a compound represented by the following formula (1). The second compound contains 1 mass % or more of a compound having one or more deuterium atoms in a compound represented by the following formula (2).

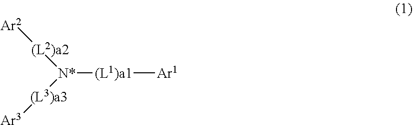

[In the formula (1),
N* is a central nitrogen atom.
L¹ to L³ each independently represent a single bond, a substituted or unsubstituted arylene group having 6 to 30 ring carbon atoms, or a substituted or unsubstituted divalent heterocyclic group having 5 to 30 ring atoms.
a1 to a3 are each independently an integer of 1 to 3.
Ar¹ to Ar³ each independently represent a substituted or unsubstituted aryl group having 6 to 30 ring carbon atoms or a substituted or unsubstituted monovalent heterocyclic group having 5 to 30 ring atoms.]

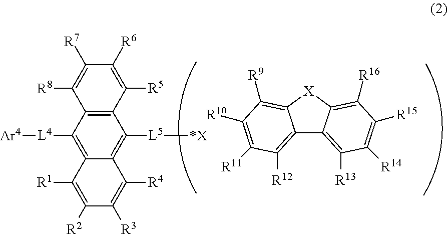

[In the formula (2),
X represents an oxygen atom, a sulfur atom, or NR¹⁰⁰.

L⁴ and L⁵ each independently represent a single bond, a substituted or unsubstituted arylene group having 6 to 30 ring carbon atoms, or a substituted or unsubstituted divalent heterocyclic group having 5 to 30 ring atoms.

Ar⁴ represents a substituted or unsubstituted aryl group having 6 to 30 ring carbon atoms or a substituted or unsubstituted monovalent heterocyclic group having 5 to 30 ring atoms.

One of R⁹ to R¹⁶ and R¹⁰⁰ is bonded to *x.

R¹ to R⁸, and R⁹ to R¹⁶ and R¹⁰⁰ that are not bonded to *x each independently represent a hydrogen atom or a substituent A.

The substituent A is a substituted or unsubstituted alkyl group having 1 to 50 carbon atoms or a substituted or unsubstituted aryl group having 6 to 50 ring carbon atoms.

Adjacent two selected from R⁹ to R¹⁶ that are not bonded to *x may form a ring or may not form a ring.]

In another aspect, the present invention provides an electronic device including the organic electroluminescent element.

### Advantageous Effects of Invention

An organic EL element in which an organic layer disposed between an anode and a cathode and including a light emitting zone includes a first layer containing a first compound containing a compound having one or more deuterium atoms represented by the formula (1) and a second layer containing a second compound containing a compound having one or more deuterium atoms represented by the formula (2) can be made into a high-performance organic EL element. More specifically an organic EL element capable of achieving a longer lifetime can be obtained. In addition, an electronic device including the high-performance organic EL element can be provided.

### BRIEF DESCRIPTION OF DRAWINGS

FIG. 1 is a schematic view showing an example of a layer configuration of an organic EL element according to an embodiment of the present invention.

FIG. 2 is a schematic view showing another example of the layer configuration of the organic EL element according to the embodiment of the invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A)

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

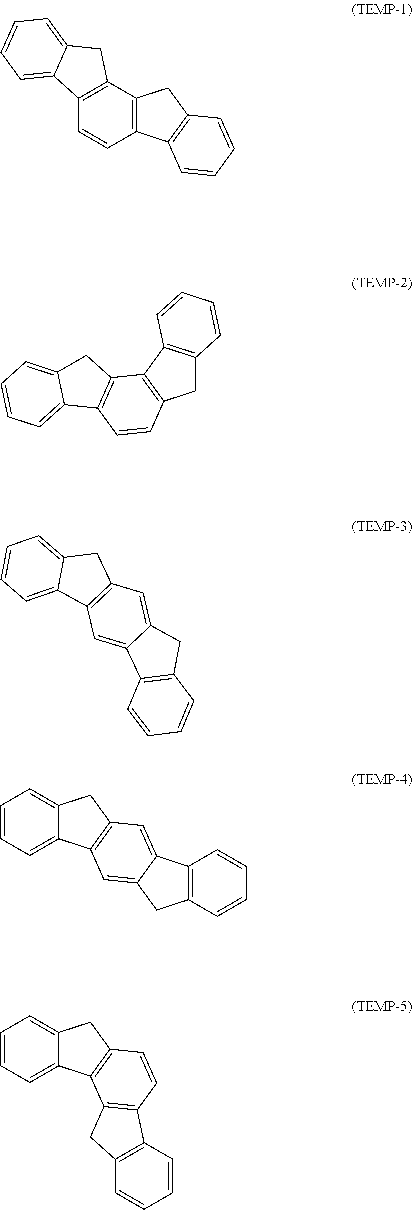

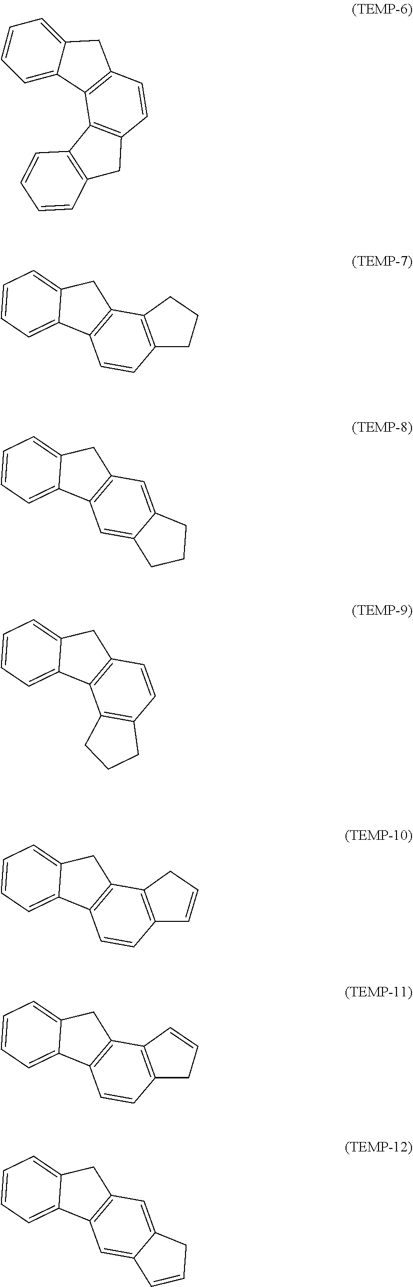

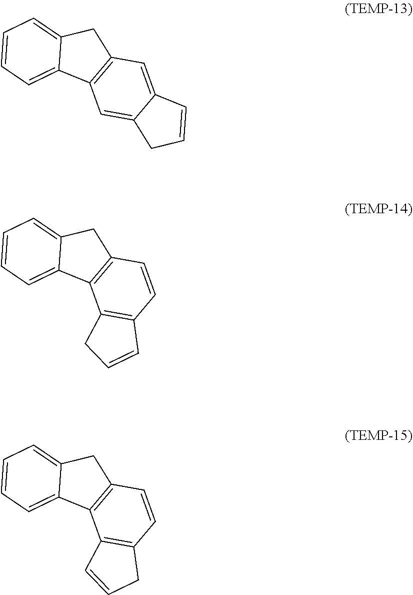

### Substituted Aryl Group (Set of Specific Examples G1B)

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

### Unsubstituted Heterocyclic Group Containing Nitrogen Atom (Set of Specific Examples G2A1)

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing Oxygen Atom (Set of Specific Examples G2A2)

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing Sulfur Atom (Set of Specific Examples G2A3)

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

### Monovalent Heterocyclic Group Derived by Removing One Hydrogen Atom from Ring Structures Represented by General Formulae (TEMP-16) to (TEMP-33) (Set of Specific Examples G2A4)

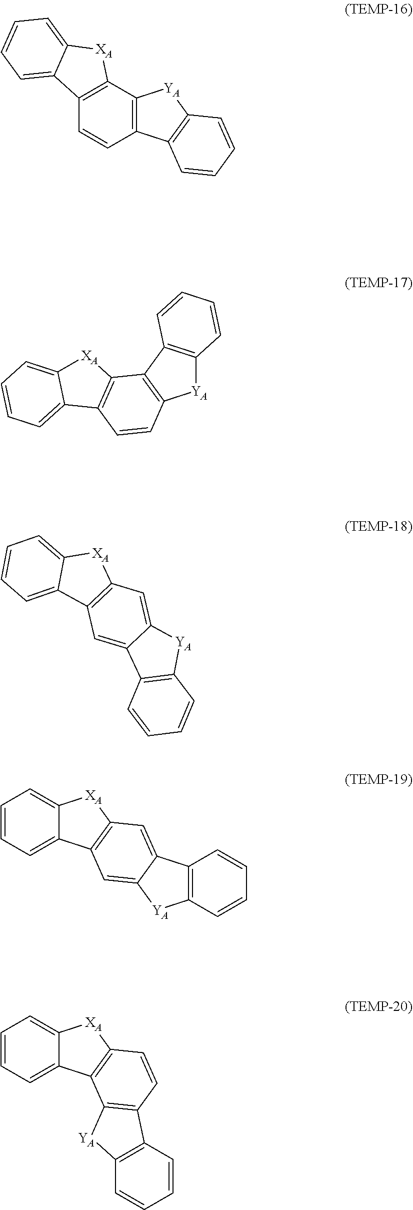

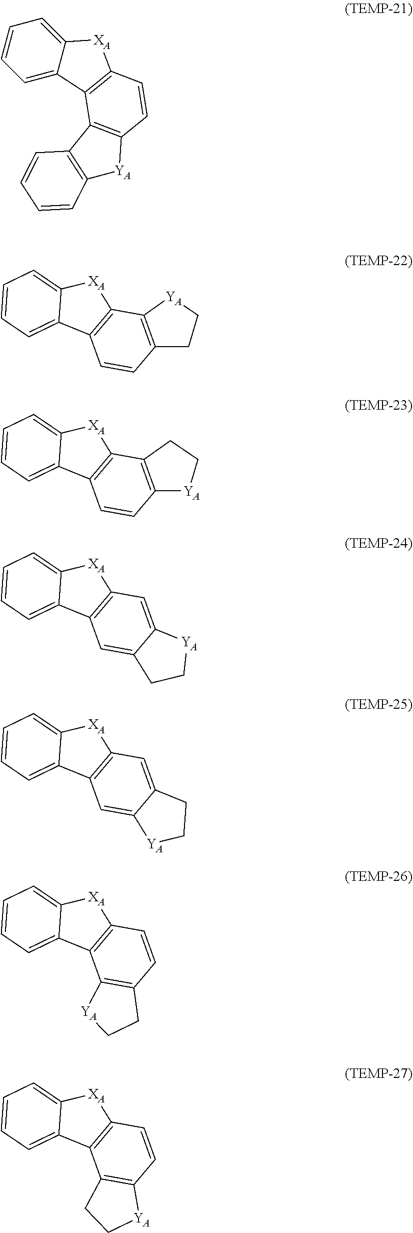

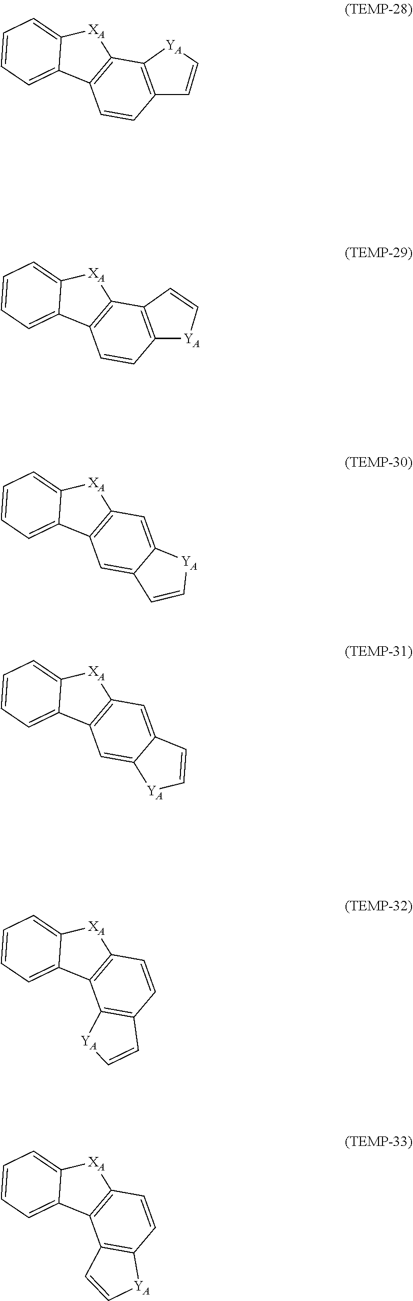

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

### Substituted Heterocyclic Group Containing Nitrogen Atom (Set of Specific Examples G2B1)

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.

### Substituted Heterocyclic Group Containing Oxygen Atom (Set of Specific Examples G2B2)

a phenyldibenzofuranyl group,
a methyldibenzofuranyl group,
a t-butyldibenzofuranyl group, and
a monovalent residual group of spiro[9H-xanthene-9,9′-[9H]fluorene].

### Substituted Heterocyclic Group Containing Sulfur Atom (Set of Specific Examples G2B3)

a phenyldibenzothiophenyl group, a methyldibenzothiophenyl group, a t-butyldibenzothiophenyl group, and a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A}represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A)

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B)

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A)

a vinyl group,
an allyl group,
a 1-butenyl group,
a 2-butenyl group, and
a 3-butenyl group.

### Substituted Alkenyl Group (Set of Specific Examples G4B)

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A)

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A)

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B)

a 4-methylcyclohexyl group.
   Group represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

Group represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G1),
—N(G2)(G2),
—N(G1)(G2),
—N(G3)(G3), and
—N(G6)(G6).

G1 represents the “substituted or unsubstituted aryl group” described in the set of specific examples G1,

G2 represents the “substituted or unsubstituted heterocyclic group” described in the set of specific examples G2,

G3 represents the “substituted or unsubstituted alkyl group” described in the set of specific examples G3, and

G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

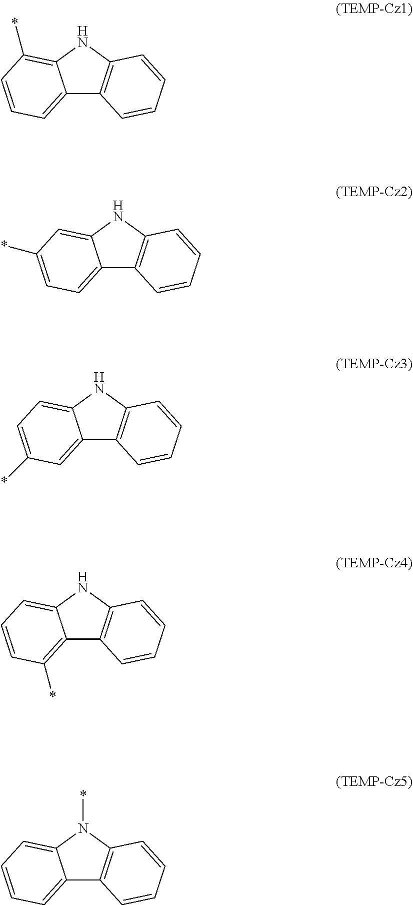 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

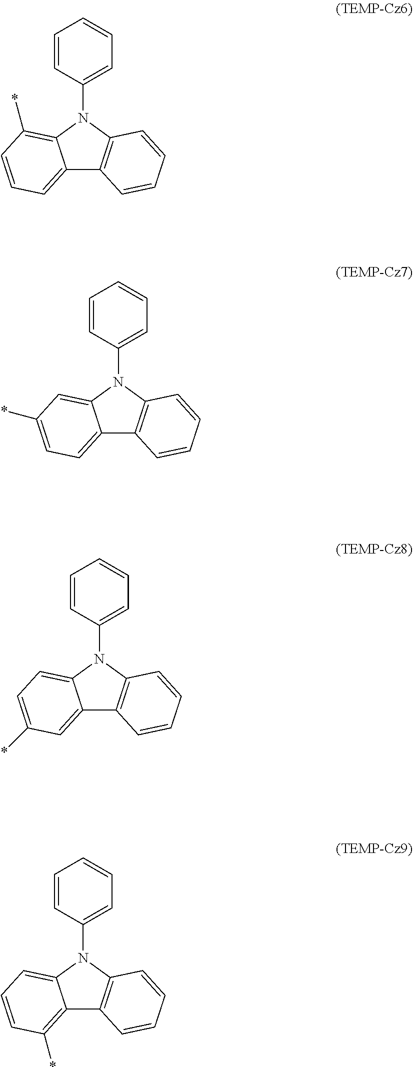 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

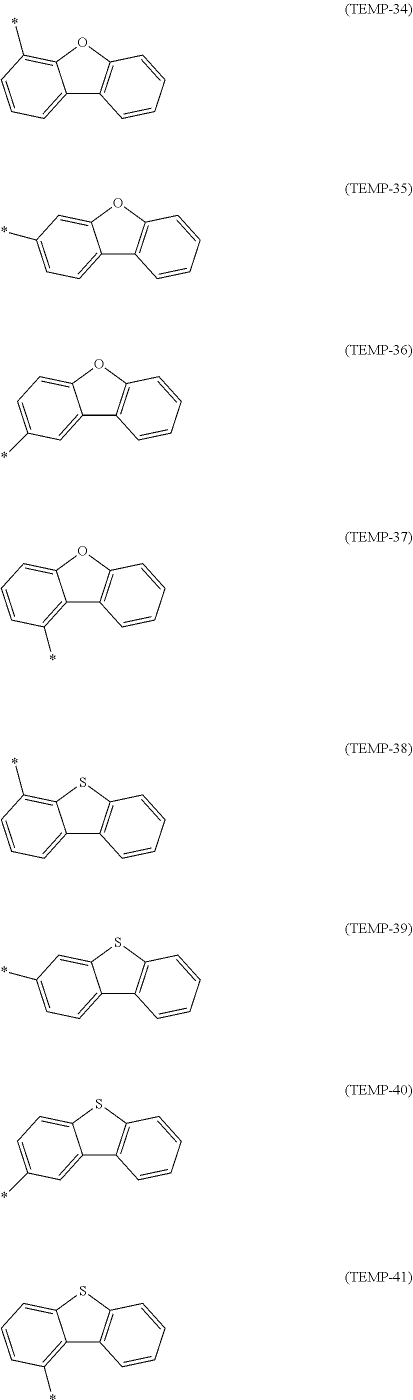

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

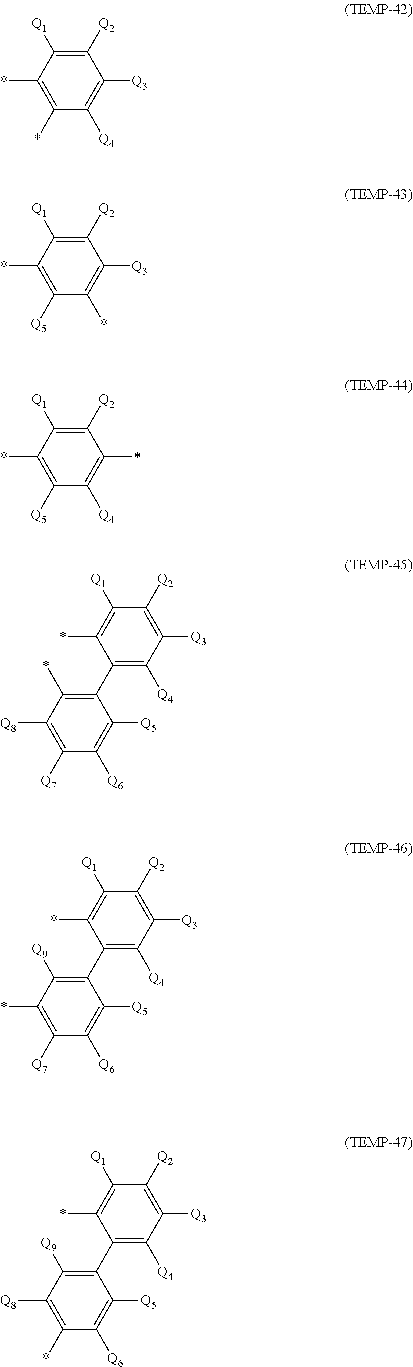

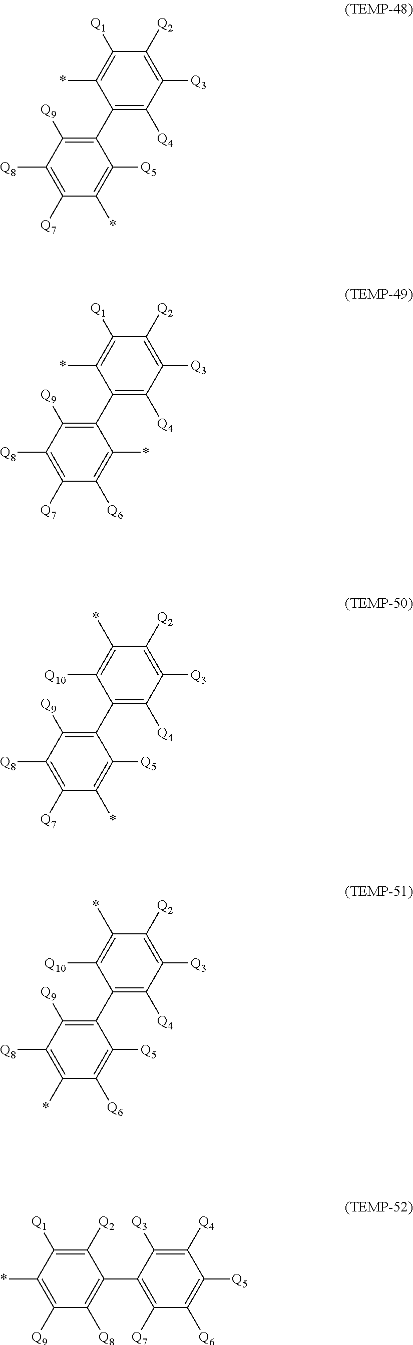

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

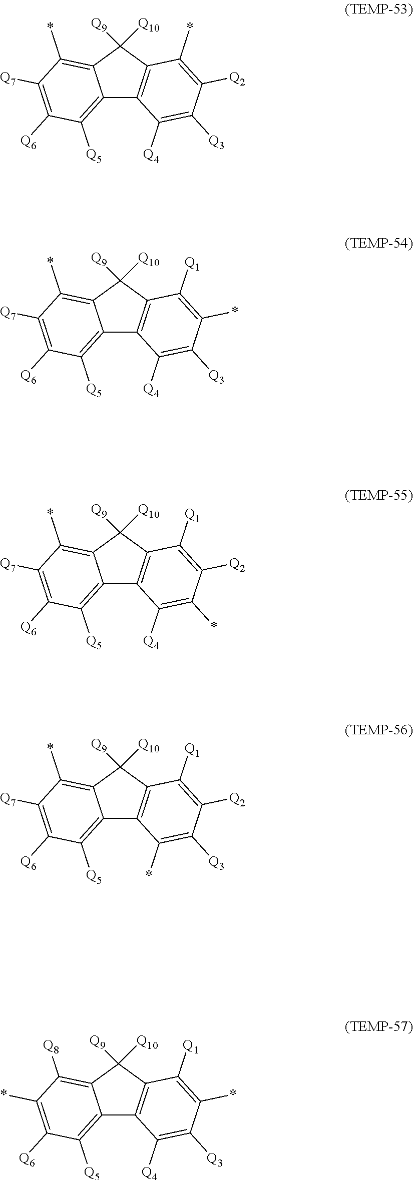

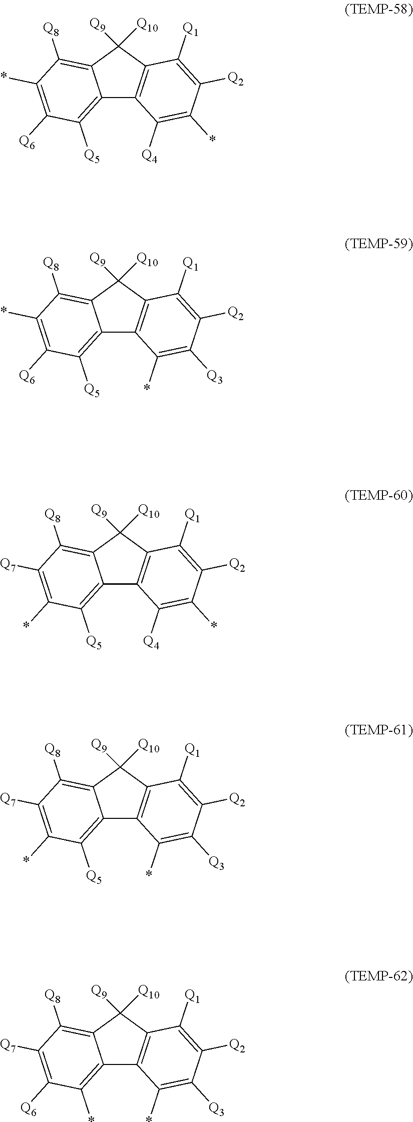

In the general formulae (TEMP-53) to (TEMP-62), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

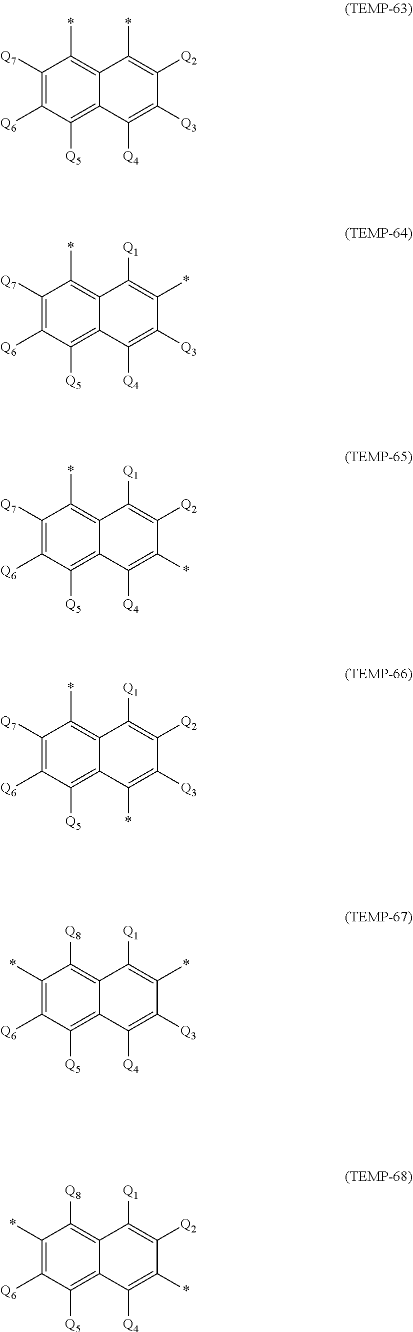

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

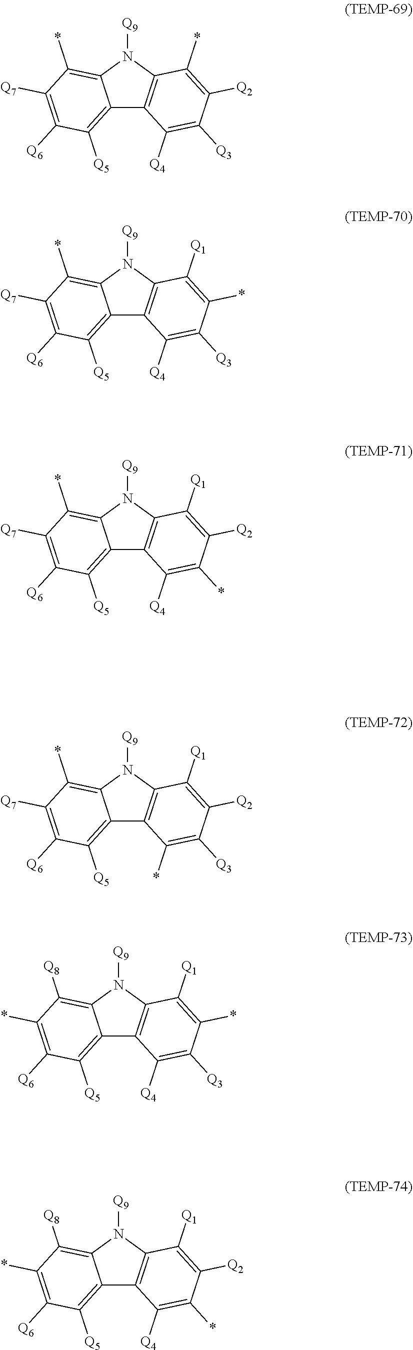

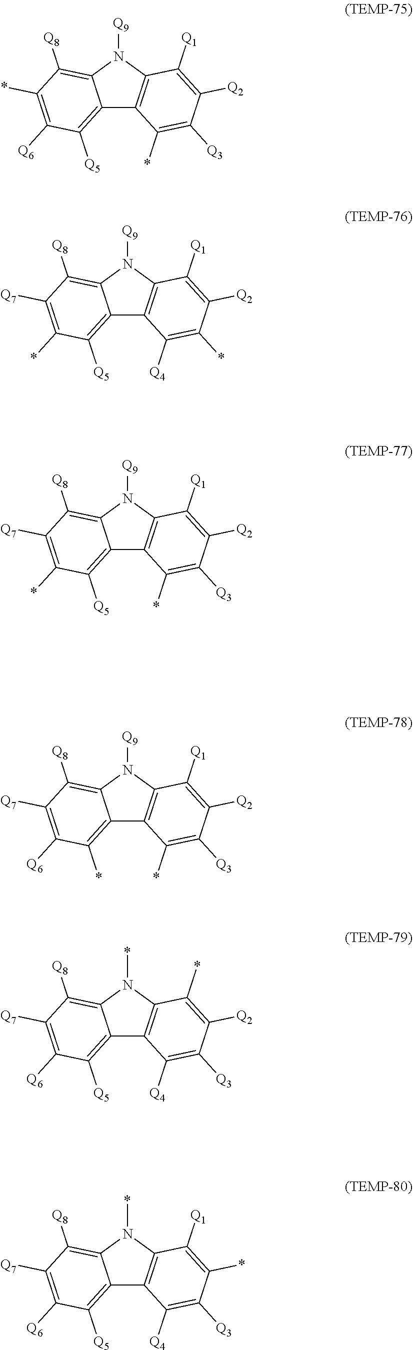

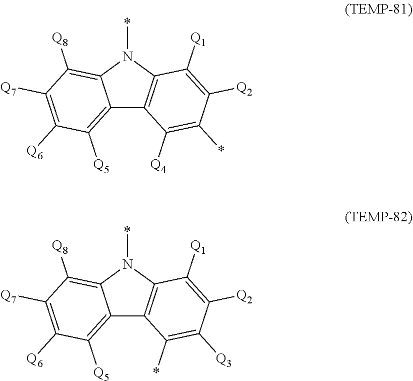

In the general formulae (TEMP-69) to (TEMP-82), Q₁ to Q₉ each independently represent a hydrogen atom or a substituent.

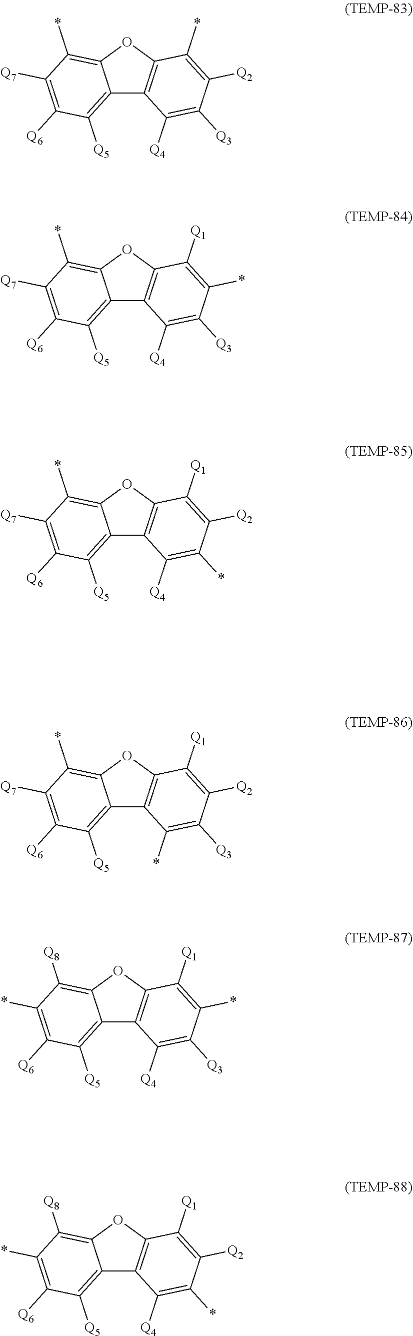

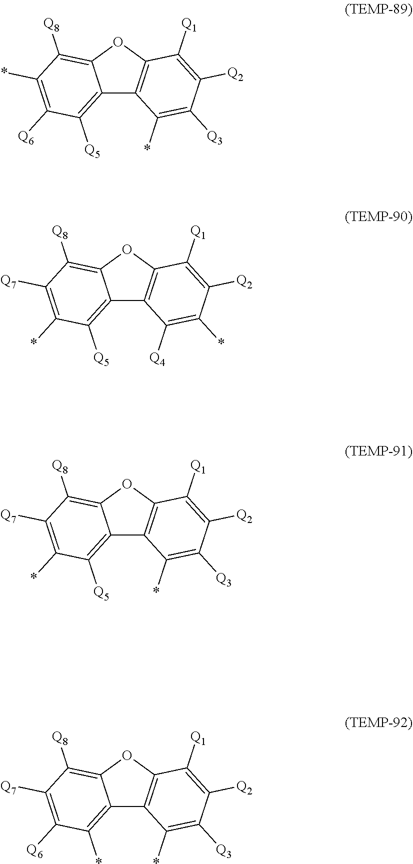

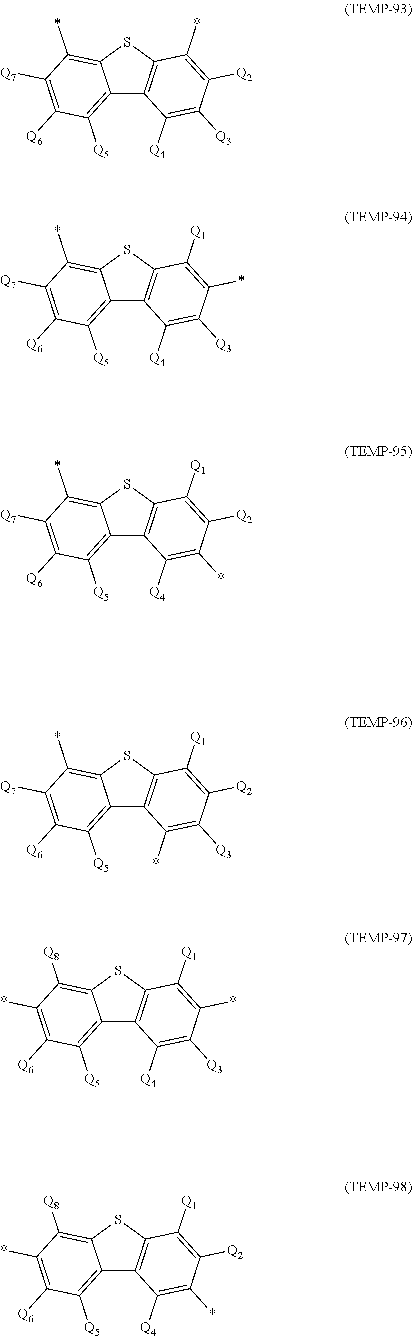

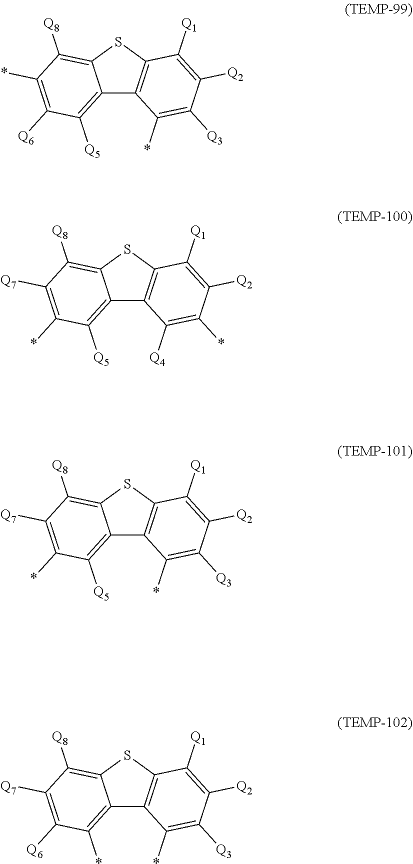

In the general formulae (TEMP-69) to (TEMP-82), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

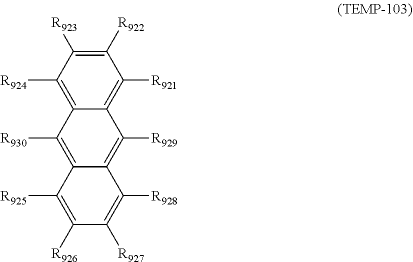

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

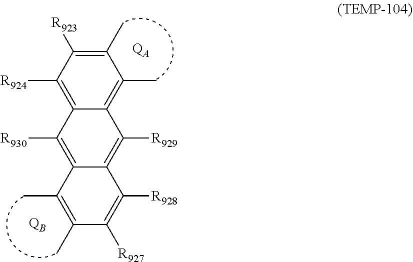

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

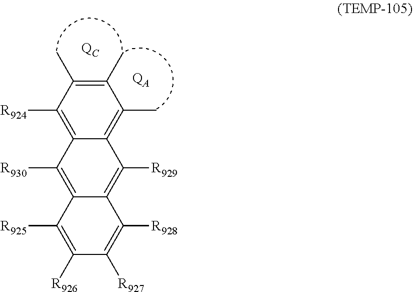

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆) (R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

In one embodiment, the substituent for the case of “substituted or unsubstituted” may be at least one substituent selected from the group consisting of
a cyano group,
a halogen atom,
an alkyl group having 1 to 30 carbon atoms,
a cycloalkyl group having 3 to 30 ring carbon atoms,
an aralkyl group having 7 to 36 carbon atoms,
an alkoxy group having 1 to 30 carbon atoms,
an aryloxy group having 6 to 30 ring carbon atoms,
a tri-substituted silyl group having a substituent selected from an alkyl group having 1 to 30 carbon atoms,
a haloalkyl group having 1 to 30 carbon atoms,
an aryl group having 6 to 30 ring carbon atoms,
an alkylthio group having 1 to 30 carbon atoms, and
an arylthio group having 6 to 30 ring carbon atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

### [Organic EL Element]

An organic EL element according to an embodiment of the present invention includes: an anode; a cathode; and an organic layer disposed between the anode and the cathode and including a light emitting zone. The organic layer includes a first layer containing a first compound and a second layer containing a second compound. The first layer and the second layer are different layers. The first compound contains 1 mass % or more of a compound having one or more deuterium atoms in a compound represented by the following formula (1). The second compound contains 1 mass % or more of a compound having one or more deuterium atoms in a compound represented by the following formula (2).

Hereinafter, the compound represented by the formula (1) may be referred to as a “compound (1)”, and the compound represented by the formula (2) may be referred to as a “compound (2)”. The compound (1) and the compound (2) will be described later.

The organic EL element according to one aspect of the present invention exhibits a high element performance by having the above-described configuration. Specifically an organic EL element having a longer lifetime can be provided.

According to one aspect of the present invention, it is possible to provide a method for improving an organic EL element performance by containing the compound (1) having at least one deuterium atom in the first layer and containing the compound (2) having at least one deuterium atom in the organic layer including the light emitting zone in the organic EL element. This method can improve the organic EL element performance, for example, as compared with at least one of the case where a compound (1) having only a protium atom as a hydrogen atom is used as a hole transport material contained in a hole transport layer which is one layer constituting the organic layer and the case where a compound (2) having only a protium atom as a hydrogen atom is used as a host material contained in the light emitting zone constituting at least a part of the organic layer.

Hereinafter, a compound represented by the formula (1) and having a structure having only a protium atom as a hydrogen atom is also referred to as a “protium body of the compound (1)”. In addition, a compound represented by the formula (2) and having a structure having only a protium atom as a hydrogen atom is also referred to as a “protium body of the compound (2)”.

The case where the protium body is used refers to, for example, at least one of the case where substantially only a protium body is used as the hole transport material contained in the hole transport layer (a content ratio of the protium body of the compound (1) to the first compound is 90 mol % or more, 95 mol % or more, or 99 mol % or more), and the case where substantially only a protium body is used as the host material contained in the light emitting zone (a content ratio of the protium body to a total protium body of the compound (2) to the second compound is 90 mol % or more, 95 mol % or more, or 99 mol % or more).

That is, for example, by using, as the hole transport material, a compound obtained by substituting at least one of the protium atoms in the protium body of the compound (1) by a deuterium atom instead of the protium body of the compound (1) or in addition to the protium body of the compound (1), and by using, as the host material contained in the light emitting zone, a compound obtained by substituting at least one of the protium atoms in the protium body of the compound (2) by a deuterium atom instead of the protium body of the compound (2) or in addition to the protium body of the compound (2), the performance can be enhanced.

A schematic configuration of the organic EL element according to one aspect of the present invention will be described with reference to the drawings.

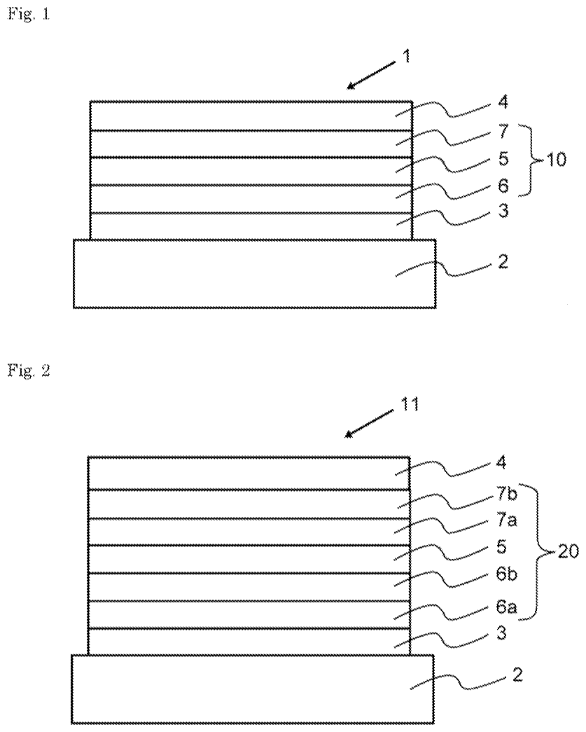

FIG. 1 is a schematic view showing an example of a configuration of the organic EL element. An organic EL element 1 shown in FIG. 1 includes a substrate 2, an anode 3, a cathode 4, and a light emitting unit 10 disposed between the anode 3 and the cathode 4. The light emitting unit 10 has a light emitting zone 5. A hole transport zone 6 (a hole injection layer, a hole transport layer, etc.) is provided between the light emitting zone 5 and the anode 3, and an electron transport zone 7 (an electron injection layer, an electron transport layer, etc.) is provided between the light emitting zone 5 and the cathode 4. An electron blocking layer (not shown) may be provided on the light emitting zone 5 on an anode 3 side, and a hole blocking layer (not shown) may be provided on the light emitting zone 5 on a cathode 4 side. Accordingly electrons and holes can be confined in the light emitting zone 5, and exciton generation efficiency in the light emitting zone 5 can be further enhanced.

FIG. 2 is a schematic view showing another configuration of the organic EL element. An organic EL element 11 shown in FIG. 2 includes the substrate 2, the anode 3, the cathode 4, and a light emitting unit 20 disposed between the anode 3 and the cathode 4. The light emitting unit 20 has the light emitting zone 5. A hole transport zone disposed between the anode 3 and the light emitting zone 5 is formed of a first hole transport layer 6a and a second hole transport layer 6b. An electron transport zone disposed between the light emitting zone 5 and the cathode 4 is formed of a first electron transport layer 7a and a second electron transport layer 7b.

In FIG. 1 and FIG. 2, the light emitting zone 5 includes at least one light emitting layer. The light emitting zone 5 may be a light emitting layer composed of a single layer, or may be a light emitting layer including a plurality of layers (for example, a plurality of light emitting layers, or a plurality of light emitting layers and space layers).

The light emitting unit 10 in the organic EL element 1 in FIG. 1 and the light emitting unit 20 in the organic EL element 11 in FIG. 2 are each an “organic layer including a light emitting zone”. The first layer included in the organic layer contains the first compound which is a compound containing 1 mass % or more of a compound having one or more deuterium atoms in the compound represented by the formula (1). Further, the second layer included in the organic layer contains the second compound which is a compound containing 1 mass % or more of a compound having one or more deuterium atoms in the compound represented by the formula (2), and the second layer is different from the first layer.

Each of the first compound contained in the first layer and the second compound contained in the second layer may be one kind alone, or may be two or more kinds.

In the organic EL element according to one aspect of the present invention, the first compound includes a plurality of compounds represented by the formula (1) and having structures different from each other, and the second compound includes a plurality of compounds represented by the formula (2) and having structures different from each other.

Hereinafter, the first compound contained in the first layer and the second compound contained in the second layer will be described. Subsequently a layer configuration of the organic EL element will be described.

### <First Compound>

As described above, the first compound contained in at least one layer of the organic layer contains 1 mass % or more of the compound having one or more deuterium atoms in the compound represented by the formula (1) (the compound (1)).

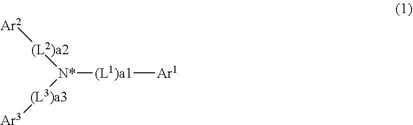

Hereinafter, symbols in the formula (1) and in each formula encompassed in the formula (1) to be described later will be described. The same symbols have the same meanings.

In the formula (1), N* is a central nitrogen atom.

In the formula (1), L¹ to L³ each independently represent a single bond, a substituted or unsubstituted arylene group having 6 to 30 ring carbon atoms, or a substituted or unsubstituted divalent heterocyclic group having 5 to 30 ring atoms.

Details of the substituted or unsubstituted arylene group having 6 to 30 ring carbon atoms and represented by L¹ to L³ are as described above in the section “Substituents in Description”.

The substituted or unsubstituted arylene group represented by L¹ to L³ is each independently preferably a phenylene group, a biphenylene group, or a terphenylene group.

Details of the substituted or unsubstituted divalent heterocyclic group having 5 to 30 ring atoms and represented by L¹ to L³ are as described above in the section “Substituents in Description”.

a1 to a3 are each independently an integer of 1 to 3.

Ar¹ to Ar³ each independently represent a substituted or unsubstituted aryl group having 6 to 30 ring carbon atoms or a substituted or unsubstituted monovalent heterocyclic group having 5 to 30 ring atoms.

In the formula (1), Ar¹ to Ar³ each independently preferably represent a substituted or unsubstituted aryl group having 6 to 16 ring carbon atoms or a substituted or unsubstituted monovalent heterocyclic group having 5 to 30 ring atoms.

When Ar¹ to Ar³ all represent a substituted or unsubstituted aryl group having 6 to 16 ring carbon atoms, the total number of carbon atoms in the two substituted or unsubstituted aryl groups each having 6 to 16 ring carbon atoms is preferably 12 to 38.

Here, in the description herein, the “total number of carbon atoms” also includes the number of carbon atoms in a substituent.

A substituent for Ar¹, a substituent for Ar², and a substituent for Ar³ are all unsubstituted substituents. That is, substitution of the substituent for Ar¹, substitution of the substituent for Ar², and substitution of the substituent for Ar³ are not permitted.

When the substituted or unsubstituted aryl group having 6 to 16 ring carbon atoms and represented by Ar¹ to Ar³ includes a substituted or unsubstituted fluorenyl group, at least one of the substituted or unsubstituted fluorenyl groups is preferably represented by the following formula (A).

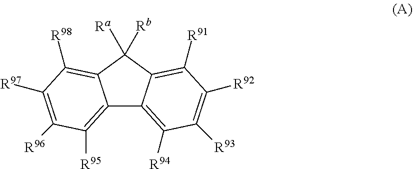

In the formula (A), R^{a} represents a substituted or unsubstituted alkyl group or a substituted or unsubstituted aryl group.

In the formula (A), R^{b} represents a substituted or unsubstituted alkyl group.

In the formula (A), one of R⁹¹ to R⁹⁸ is a single bond bonded to (L¹)a1, (L²)a2, or (L³)a3.

R⁹¹ to R⁹⁸ which are not the single bond each independently represent a hydrogen atom or a substituent, and the substituent is a substituent same as the substituent for Ar¹, the substituent for Ar², and the substituent for Ar³ in the formula (1), and a preferred aspect is also the same.

Details of the substituted or unsubstituted alkyl group represented by R^{a} and R^{b} are as described above in the section “Substituents in Description”.

Substituents for the substituted alkyl group represented by R^{a} and R^{b} are all unsubstituted substituents. That is, substitutions of the substituents for the substituted alkyl group represented by R^{a} and R^{b} are not permitted.

The unsubstituted alkyl group represented by R^{a} and R^{b} is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and even more preferably a methyl group or a t-butyl group.

Details of the substituted or unsubstituted aryl group represented by R^{a} are as described above in the section “Substituents in Description”.

A substituent for the substituted aryl group represented by R^{a} is an unsubstituted substituent. That is, substitution of the substituent for the substituted aryl group represented by R^{a} is not permitted.

The unsubstituted aryl group represented by R^{a} is more preferably selected from the group consisting of a phenyl group, a biphenyl group, a naphthyl group, and a phenanthryl group.

Aryl Group Represented by Ar¹ to Ar³

Details of the substituted or unsubstituted aryl group having 6 to 16 ring carbon atoms and represented by Ar¹ to Ar³ are as described above in the section “Substituents in Description”.

The unsubstituted aryl group represented by Ar¹ to Ar³ preferably includes only a benzene ring, and is more preferably selected from the group consisting of a phenyl group, a biphenyl group, a naphthyl group, and a phenanthryl group.

Heterocyclic Group Represented by Ar¹ to Ar³

Details of the substituted or unsubstituted monovalent heterocyclic group having 5 to 30 ring atoms and represented by Ar¹ to Ar³ are as described above in the section “Substituents in Description”.

The unsubstituted monovalent heterocyclic group represented by Ar¹ to Ar³ is preferably a heterocyclic group selected from a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, a dibenzofuranyl group, a naphthobenzofuranyl group, a dibenzothiophenyl group, and a naphthobenzothiophenyl group, and more preferably a heterocyclic group selected from a pyridyl group, a dibenzofuranyl group, and a dibenzothiophenyl group.

Substituent for Ar¹, Substituent for Ar², and Substituent for Ar³

The substituent for Ar¹, the substituent for Ar², and the substituent for Ar³ are each independently
a halogen atom, a nitro group, a cyano group,
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
an unsubstituted haloalkyl group having 1 to 50 carbon atoms,
an unsubstituted alkoxy group having 1 to 50 carbon atoms,
an unsubstituted haloalkoxy group having 1 to 50 carbon atoms,
an unsubstituted alkylthio group having 1 to 50 carbon atoms,
an unsubstituted aryl group having 6 to 50 ring carbon atoms,
an unsubstituted aryloxy group having 6 to 50 ring carbon atoms,
an unsubstituted arylthio group having 6 to 50 ring carbon atoms,
an unsubstituted aralkyl group having 7 to 50 carbon atoms,
an unsubstituted monovalent heterocyclic group having 5 to 50 ring atoms, or
a mono-, di-, or tri-substituted silyl group having a substituent selected from an unsubstituted alkyl group having 1 to 50 carbon atoms, an unsubstituted cycloalkyl group having 3 to 50 ring carbon atoms, an unsubstituted aryl group having 6 to 50 ring carbon atoms, and an unsubstituted monovalent heterocyclic group having 5 to 50 ring atoms.

### Halogen Atom

Details of the halogen atom as the substituent for Ar¹, the substituent for Ar², and the substituent for Ar³ are as described above in the section “Substituents in Description”.

### Alkyl Group

Details of the unsubstituted alkyl group having 1 to 50 carbon atoms as the substituent for Ar¹, the substituent for Ar², and the substituent for Ar³ are as described above in the section “Substituents in Description”.

### Alkenyl Group

Details of the unsubstituted alkenyl group having 2 to 50 carbon atoms as the substituent for Ar¹, the substituent for Ar², and the substituent for Ar³ are as described above in the section “Substituents in Description”.

### Alkynyl Group

Details of the unsubstituted alkynyl group having 2 to 50 carbon atoms as the substituent for Ar¹, the substituent for Ar², and the substituent for Ar³ are as described above in the section “Substituents in Description”.

### Cycloalkyl Group

Details of the unsubstituted cycloalkyl group having 3 to 50 ring carbon atoms as the substituent for Ar¹, the substituent for Ar², and the substituent for Ar³ are as described above in the section “Substituents in Description”.

### Haloalkyl Group

Details of the unsubstituted haloalkyl group having 1 to 50 carbon atoms as the substituent for Ar¹, the substituent for Ar², and the substituent for Ar³ are as described above in the section “Substituents in Description”.

### Alkoxy Group

Details of the unsubstituted alkoxy group having 1 to 50 carbon atoms as the substituent for Ar¹, the substituent for Ar², and the substituent for Ar³ are as described above in the section “Substituents in Description”.

### Haloalkoxy Group

The unsubstituted haloalkoxy group having 1 to 50 carbon atoms as the substituent for Ar¹, the substituent for Ar², and the substituent for Ar³ is a group represented by —O(G12), and G12 is the unsubstituted haloalkyl group.

### Alkylthio Group

Details of the unsubstituted alkylthio group having 1 to 50 carbon atoms as the substituent for Ar¹, the substituent for Ar², and the substituent for Ar³ are as described above in the section “Substituents in Description”.

### Aryl Group

Details of the unsubstituted aryl group having 6 to 50 ring carbon atoms as the substituent for Ar¹, the substituent for Ar², and the substituent for Ar³ are as described above in the section “Substituents in Description”.

The unsubstituted aryl group as the substituent for Ar¹, the substituent for Ar², and the substituent for Ar³ is more preferably selected from the group consisting of a phenyl group, a naphthyl group, and a phenanthryl group.

The unsubstituted aryl group having 6 to 50 ring carbon atoms as the substituent for Ar² and the substituent for Ar³ includes, for example, a condensed aryl group such as a fluorenyl group, a phenanthryl group, and an anthracenyl group, but does not include a set of rings such as a biphenyl group, a terphenyl group, and a naphthylphenyl group.

### Aryloxy Group

Details of the unsubstituted aryloxy group having 6 to 50 ring carbon atoms as the substituent for Ar¹, the substituent for Ar², and the substituent for Ar³ are as described above in the section “Substituents in Description”.

### Arylthio Group

Details of the unsubstituted arylthio group having 6 to 50 ring carbon atoms as the substituent for Ar¹, the substituent for Ar², and the substituent for Ar³ are as described above in the section “Substituents in Description”.

### Aralkyl Group

Details of the unsubstituted aralkyl group having 7 to 50 carbon atoms as the substituent for Ar¹, the substituent for Ar², and the substituent for Ar³ are as described above in the section “Substituents in Description”.

### Heterocyclic Group

Details of the unsubstituted monovalent heterocyclic group having 5 to 50 ring atoms as the substituent for Ar¹, the substituent for Ar², and the substituent for Ar³ are as described above in the section “Substituents in Description”.

### Substituted Silyl Group

Details of the substituent for the mono-, di-, or tri-substituted silyl group as the substituent for Ar¹, the substituent for Ar², and the substituent for Ar³ are as described above in the section “Substituents in Description”.

Ar¹ is preferably substituted with an unsubstituted alkyl group having 1 to 50 carbon atoms.

In one aspect of the present invention, the total number of carbon atoms in respective groups bonded to the central nitrogen atom in the compound (1) is 10 or more. The total number of carbon atoms in respective groups bonded to the central nitrogen atom in the compound (1) is preferably 12 or more, and more preferably 18 or more.

In one aspect of the present invention, the compound (1) is represented by any one of the following formula (1-1a) to formula (1-1c).

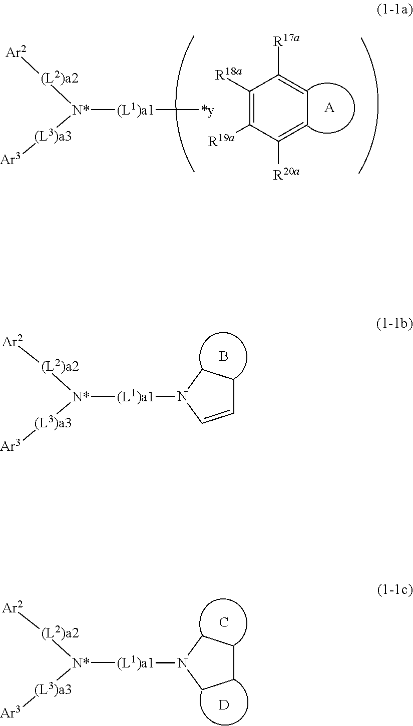

In the formula (1-1a), a ring A is a substituted or unsubstituted aromatic hydrocarbon ring having 6 to 50 ring carbon atoms or a substituted or unsubstituted heterocyclic ring having 5 to 50 ring atoms.

The substituted or unsubstituted aromatic hydrocarbon ring having 6 to 50 ring carbon atoms and represented by the ring A is more preferably selected from the group consisting of a substituted or unsubstituted phenyl group, biphenyl group, naphthyl group, and phenanthryl group.

The substituted or unsubstituted heterocyclic ring having 5 to 50 ring atoms and represented by the ring A is preferably a heterocyclic group selected from a substituted or unsubstituted pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, dibenzofuranyl group, naphthobenzofuranyl group, dibenzothiophenyl group, and naphthobenzothiophenyl group, and more preferably a heterocyclic group selected from a carbazolyl group, a dibenzofuranyl group, and a dibenzothiophenyl group.

In the formula (1-1a), one of R^{17a} to R^{20a} is bonded to *y. R^{17a} to R^{20a} that are not bonded to *7 each independently represent a hydrogen atom or a substituent B.

The substituent B is a substituted or unsubstituted alkyl group having 1 to 50 carbon atoms or a substituted or unsubstituted aryl group having 6 to 50 ring carbon atoms.

Details of the substituted or unsubstituted alkyl group having 1 to 50 carbon atoms and represented by the substituent B are as described above in the section “Substituents in Description”.

Details of the substituted or unsubstituted aryl group having 6 to 50 ring carbon atoms and represented by the substituent B are as described above in the section “Substituents in Description”.

In the formula (1-1a), adjacent two selected from R^{17a} to R^{20a} that are not bonded to *y may form a ring or may not form a ring.

In the formula (1-1b), a ring B is a substituted or unsubstituted aromatic hydrocarbon ring having 6 to 50 ring carbon atoms or a substituted or unsubstituted heterocyclic ring having 5 to 50 ring atoms.

The substituted or unsubstituted aromatic hydrocarbon ring having 6 to 50 ring carbon atoms and the substituted or unsubstituted heterocyclic ring having 5 to 50 ring atoms, which are represented by the ring B, are the same as those described for the ring A.

In the formula (1-1c), a ring C and a ring D are each independently a substituted or unsubstituted aromatic hydrocarbon ring having 6 to 50 ring carbon atoms or a substituted or unsubstituted heterocyclic ring having 5 to 50 ring atoms.

The substituted or unsubstituted aromatic hydrocarbon ring having 6 to 50 ring carbon atoms and the substituted or unsubstituted heterocyclic ring having 5 to 50 ring atoms, which are represented by the ring C and the ring D, are the same as those described for the ring A.

In the formula (1-1a) to the formula (1-1c), N*, L¹ to L³, Ar², Ar³, and a1 to a3 are as defined in the formula (1).

In one aspect of the present invention, the compound (1) is represented by the following formula (1-2).

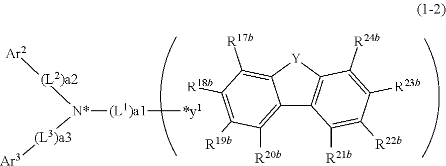

In the formula (1-2), Y represents an oxygen atom, a sulfur atom, NR¹⁰¹, or CR¹⁰²R¹⁰³.

In the formula (1-2), one of R^{17b} to R^{24b} and R¹⁰¹ is bonded to *y¹. R¹⁰², R¹⁰³, and R^{17b} to R^{24b} and R¹⁰¹ that are not bonded to *y¹ each independently represent a hydrogen atom or a substituent C.

The substituent C is a substituted or unsubstituted alkyl group having 1 to 50 carbon atoms or a substituted or unsubstituted aryl group having 6 to 50 ring carbon atoms.

Details of the substituted or unsubstituted alkyl group having 1 to 50 carbon atoms and represented by the substituent C are as described above in the section “Substituents in Description”.

Details of the substituted or unsubstituted aryl group having 6 to 50 ring carbon atoms and represented by the substituent C are as described above in the section “Substituents in Description”.

In the formula (1-2), N*, L¹ to L³, Ar², Ar³, and a1 to a3 are as defined in the formula (1).

In one aspect of the present invention, R^{17b} to R^{24b} that are not bonded to L¹ in the formula (1-2) each independently represent a hydrogen atom or an alkyl group having 1 to 10 carbon atoms, and at least one of R^{17b} to R²⁴ that are not bonded to L¹ represents an alkyl group having 1 to 10 carbon atoms.

In one aspect of the present invention, the compound (1) is represented by the following formula (1-3a) or formula (1-3b).

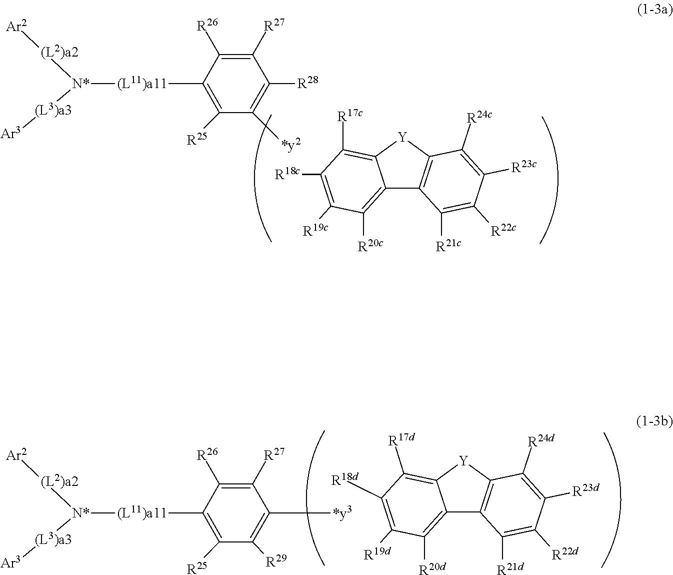

In the formula (1-3a) and the formula (1-3b), L¹¹ represents a single bond, a substituted or unsubstituted arylene group having 6 to 30 ring carbon atoms, or a substituted or unsubstituted divalent heterocyclic group having 5 to 30 ring atoms.

In the formula (1-3a) and the formula (1-3b), all is an integer of 1 or 2. Y represents an oxygen atom, a sulfur atom, NR¹⁰¹, or CR¹⁰²R¹⁰³.

In the formula (1-3a), one of R^{17c} to R^{24c} and R¹⁰¹ is bonded to *y², and R¹⁰², R¹⁰³, and R^{17c} to R^{24c} and R¹⁰¹ that are not bonded to *y² each independently represent a hydrogen atom or a substituent C.

In the formula (1-3b), one of R^{17d} to R^{24d} and R¹⁰¹ is bonded to *y³, and R¹⁰², R¹⁰³, and R^{17d} to R^{24d} and R¹⁰¹ that are not bonded to *y³ each independently represent a hydrogen atom or a substituent C.

R²⁵ to R²⁸ in the formula (1-3a) and R²⁵ to R²⁷ and R²⁹ in the formula (1-3b) each independently represent a hydrogen atom or a substituent D.

The substituent D is a substituted or unsubstituted alkyl group having 1 to 50 carbon atoms or a substituted or unsubstituted aryl group having 6 to 50 ring carbon atoms.

Details of the substituted or unsubstituted alkyl group having 1 to 50 carbon atoms and represented by the substituent D are as described above in the section “Substituents in Description”.

Details of the substituted or unsubstituted aryl group having 6 to 50 ring carbon atoms and represented by the substituent D are as described above in the section “Substituents in Description”.

R²⁶ and R²⁷, R²⁷ and R²⁸ in the formula (1-3a), and R²⁶ and R²⁷, R²⁵ and R²⁹ in the formula (1-3b) may each independently form a ring or may not form a ring.

R²⁵ or R²⁶ in the formula (1-3a) and the formula (1-3b) may or may not be bonded to a ring carbon atom in L¹¹ adjacent thereto.

N*, L², L³, Ar², Ar³, a2, and a3 are as defined in the formula (1). The substituent C is as defined in the formula (1-2).

In one aspect of the present invention, the compound represented by the formula (1) is represented by the formula (1-3a).

In one aspect of the present invention, the compound (1) is represented by the following formula (1-4).

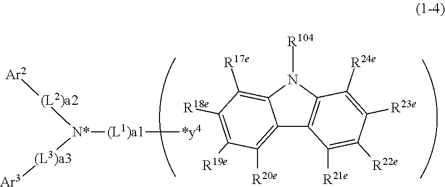

In the formula (1-4), R¹⁰⁴ represents a hydrogen atom or a substituent E.

The substituent E is a substituted or unsubstituted alkyl group having 1 to 50 carbon atoms or a substituted or unsubstituted aryl group having 6 to 50 ring carbon atoms.

Details of the substituted or unsubstituted alkyl group having 1 to 50 carbon atoms and represented by the substituent E are as described above in the section “Substituents in Description”.

Details of the substituted or unsubstituted aryl group having 6 to 50 ring carbon atoms and represented by the substituent E are as described above in the section “Substituents in Description”.

Y represents an oxygen atom, a sulfur atom, NR¹⁰¹, or CR¹⁰²R¹⁰³.

One of R^{17e} to R^{24e} and R¹⁰¹ is bonded to *y⁴. R¹⁰², R¹⁰³, and R^{17e} to R^{24e} and R¹⁰¹ that are not bonded to *y⁴ each independently represent a hydrogen atom or a substituent C.

In the formula (1-4), N*, L¹ to L³, Ar², Ar³, and a1 to a3 are as defined in the formula (1). The substituent C is as defined in the formula (1-2).

In one aspect of the present invention, the compound (1) is represented by the following formula (1-5).

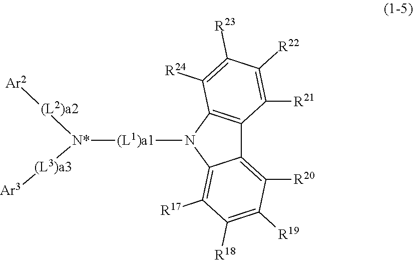

In the formula (1-5), R¹⁷ to R²⁴ each independently represent a hydrogen atom or a substituent C. N*, L¹ to L³, Ar², Ar³, and a1 to a3 are as defined in the formula (1). The substituent C is as defined in the formula (1-2).

In one aspect of the present invention, the compound (1) is represented by the following formula (1-6a) or formula (1-6b).

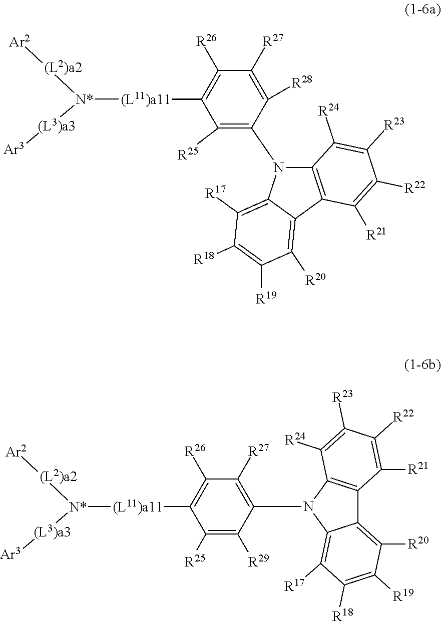

In the formula (1-6a) and the formula (1-6b), N*, L², L³, Ar², Ar³, a2, and a3 are as defined in the formula (1). R¹⁷ to R²⁴ are as defined in the formula (1-5).

R²⁵ to R²⁸ in the formula (1-6a) and R²⁵ to R²⁷ and R²⁹ in the formula (1-6b) are as defined in the formula (1-3a) and the formula (1-3b), respectively.

In one aspect of the present invention, the compound represented by the formula (1) is represented by the formula (1-6a).

### (Preferred Example of Compound (1))

Suitable examples of the compound (1) include compounds represented by the following formulae (1-7) to (1-16).

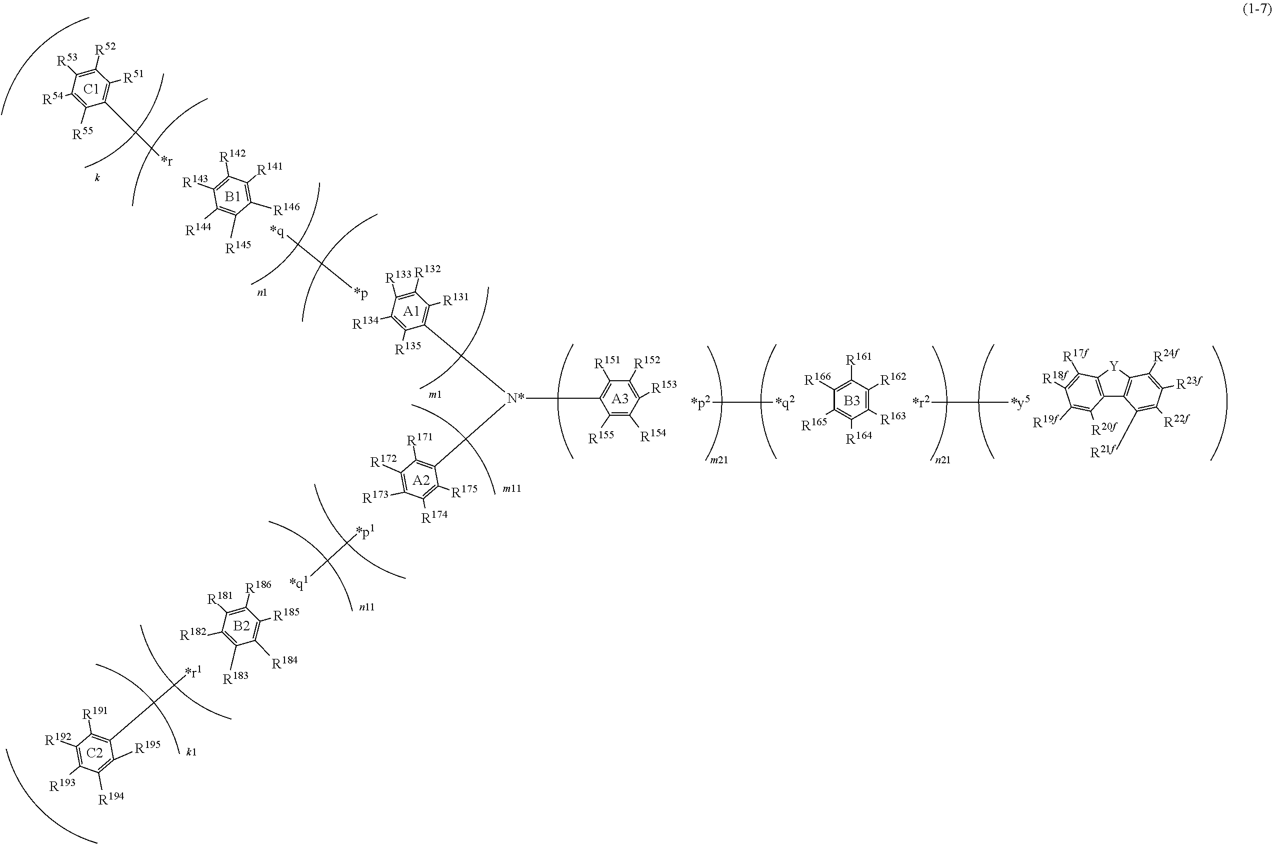

In the formula (1-7), Y represents an oxygen atom, a sulfur atom, NR¹⁰¹, or CR¹⁰²R¹⁰³.

One of R^{17f} to R^{24f} and R¹⁰¹ is bonded to *y⁵. R¹⁰², R¹⁰³, and R^{17f} to R^{24f} and R¹⁰¹ that are not bonded to *y⁵ each independently represent a hydrogen atom or the substituent C.

In the formula (1-7), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another one selected from R¹⁶¹ to R¹⁶⁶ is a single bond bonded to *r².

In the formula (1-7), R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ each independently represent a hydrogen atom or a substituent F. The substituent F is a substituent same as the substituent for Ar¹, the substituent for Ar², and the substituent for Ar³ in the formula (1), provided that
one selected from R¹³¹ to R¹³⁵ is a single bond bonded to *p,
one selected from R¹⁴¹ to R¹⁴⁶ is a single bond bonded to *q, and another one or two selected from R¹⁴¹ to R¹⁴⁶ are a single bond bonded to *r,
one selected from R¹⁷¹ to R¹⁷⁵ is a single bond bonded to *p¹, and
one selected from R¹⁸¹ to R¹⁸⁶ is a single bond bonded to *q¹, and another
one or two selected from R¹⁸¹ to R¹⁸⁶ are a single bond bonded to *r¹.

In the formula (1-7),
m1, m11, and m21 are each independently 0 or 1,
n1, n11, and n21 are each independently 0 or 1,
   when m1 is 0 and n1 is 0, *r is bonded to the central nitrogen atom N*
   when m1 is 0 and n1 is 1, *p is bonded to the central nitrogen atom N*
   when m1 is 1 and n1 is 0, one selected from R¹³¹ to R¹³⁵ is a single bond bonded to *r,
   when m11 is 0 and n11 is 0, *r¹ is bonded to the central nitrogen atom N*,
   when m11 is 0 and n11 is 1, *p¹ is bonded to the central nitrogen atom N*,
   when m11 is 1 and n11 is 0, one selected from R¹⁷¹ to R¹⁷⁵ is a single bond bonded to *r¹,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r², and
k and k1 are each independently 1 or 2.

In the formula (1-7), adjacent two selected from R¹³¹ to R¹³⁵ which are not the single bond, adjacent two selected from R¹⁴¹ to R¹⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R¹⁷¹ to R¹⁷⁵ which are not the single bond, adjacent two selected from R¹⁸¹ to R¹⁸⁶ which are not the single bond, adjacent two selected from R⁵¹ to R⁵⁵, and adjacent two selected from R¹⁹¹ to R¹⁹⁵ are each independently not bonded to each other, and therefore do not form a ring structure, and a benzene ring A1 and a benzene ring B1, the benzene ring A1 and a benzene ring C1, the benzene ring B1 and the benzene ring C1, a benzene ring A2 and a benzene ring B2, the benzene ring A2 and a benzene ring C2, the benzene ring B2 and the benzene ring C2, and a benzene ring A3 and a benzene ring B3 are not crosslinked.

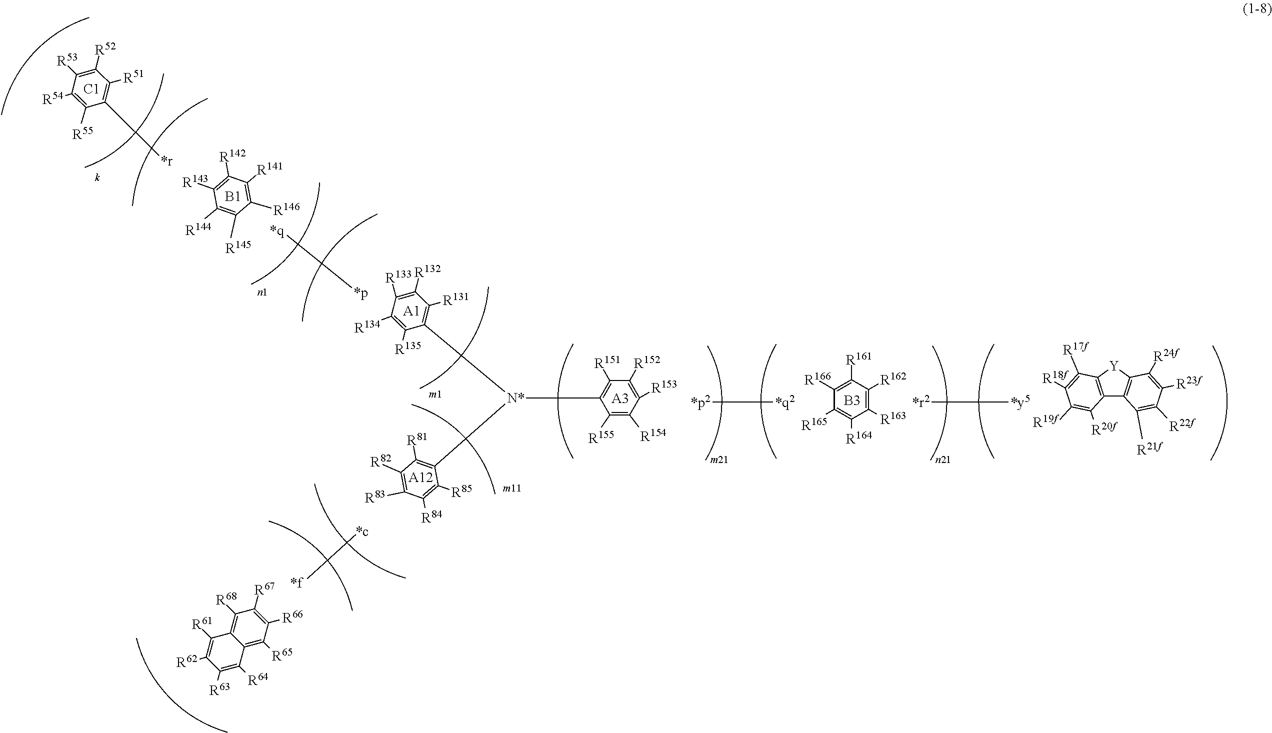

In the formula (1-8), Y and R^{17f} to R^{24f} are as described above.

In the formula (1-8), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-8), R¹³¹ to R¹³⁵, R¹⁴¹ to R¹⁴⁶, R⁸¹ to R⁸⁵, R⁴¹ to R⁴⁶, R⁵¹ to R⁵⁵, and R⁷¹ to R⁸⁰ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R¹³¹ to R¹³⁵ is a single bond bonded to *p,
one selected from R¹⁴¹ to R¹⁴⁶ is a single bond bonded to *q, and another one or two selected from R¹⁴¹ to R¹⁴⁶ are a single bond bonded to *r,
one selected from R⁸¹ to R⁸⁵ is a single bond bonded to *c, and
one selected from R⁴¹ to R⁴⁶ is a single bond bonded to *d, and another one selected from R⁴¹ to R⁴⁶ is a single bond bonded to *e.

In the formula (1-8),
m1, m3, and m21 are each independently 0 or 1,
n1, n3, and n21 are each independently 0 or 1,
   when m1 is 0 and n1 is 0, *r is bonded to the central nitrogen atom N*
   when m1 is 0 and n1 is 1, *p is bonded to the central nitrogen atom N*
   when m1 is 1 and n1 is 0, one selected from R¹³¹ to R¹³⁵ is a single bond bonded to *r,
   when m3 is 0 and n3 is 0, *e is bonded to the central nitrogen atom N*
   when m3 is 0 and n3 is 1, *c is bonded to the central nitrogen atom N*
   when m3 is 1 and n3 is 0, one selected from R⁸¹ to R⁸⁵ is a single bond bonded to *e,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r², and
k is 1 or 2.

In the formula (1-8), one selected from R⁷¹ to R⁸⁰ is a single bond bonded to *h.

In the formula (1-8), adjacent two selected from R¹³¹ to R¹³⁵ which are not the single bond, adjacent two selected from R¹⁴¹ to R¹⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R⁸¹ to R⁸⁵ which are not the single bond, adjacent two selected from R⁴¹ to R⁴⁶ which are not the single bond, adjacent two selected from R⁵¹ to R⁵⁵, and adjacent two selected from R⁷¹ to R⁸⁰ are each independently not bonded to each other, and therefore do not form a ring structure, and a benzene ring A1 and a benzene ring B1, the benzene ring A1 and a benzene ring C1, the benzene ring B1 and the benzene ring C1, a benzene ring A12 and a benzene ring B12, and a benzene ring A3 and a benzene ring B3 are not crosslinked.

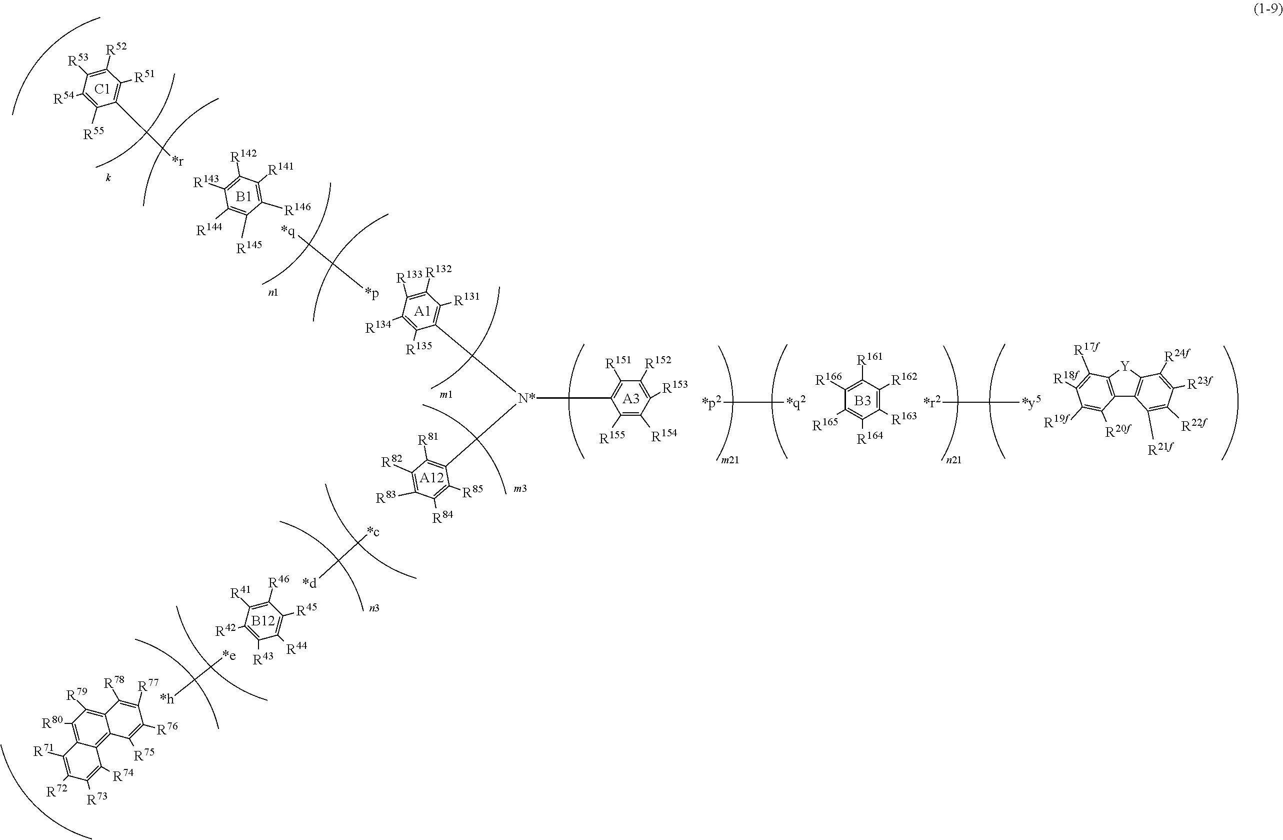

In the formula (1-9), Y and R^{17f} to R^{24f} are as described above.

In the formula (1-9), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-9), R¹³¹ to R¹³⁵, R¹⁴¹ to R¹⁴⁶, R⁸¹ to R⁸⁵, R⁴¹ to R⁴⁶, R⁵¹ to R⁵⁵, and R⁷¹ to R⁸⁰ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R¹³¹ to R¹³⁵ is a single bond bonded to *p,
one selected from R¹⁴¹ to R¹⁴⁶ is a single bond bonded to *q, and another one or two selected from R¹⁴¹ to R¹⁴⁶ are a single bond bonded to *r,
one selected from R⁸¹ to R⁸⁵ is a single bond bonded to *c, and
one selected from R⁴¹ to R⁴⁶ is a single bond bonded to *d, and another one selected from R⁴¹ to R⁴⁶ is a single bond bonded to *e.

In the formula (1-9),
m1, m3, and m21 are each independently 0 or 1,
n1, n3, and n21 are each independently 0 or 1,
   when m1 is 0 and n1 is 0, *r is bonded to the central nitrogen atom N*
   when m1 is 0 and n1 is 1, *p is bonded to the central nitrogen atom N*
   when m1 is 1 and n1 is 0, one selected from R¹³¹ to R¹³⁵ is a single bond bonded to *r,
   when m3 is 0 and n3 is 0, *e is bonded to the central nitrogen atom N*
   when m3 is 0 and n3 is 1, *c is bonded to the central nitrogen atom N*
   when m3 is 1 and n3 is 0, one selected from R⁸¹ to R⁸⁵ is a single bond bonded to *e,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r², and
k is 1 or 2.

In the formula (1-9), one selected from R⁷¹ to R⁸⁰ is a single bond bonded to *h.

In the formula (1-9), adjacent two selected from R¹³¹ to R¹³⁵ which are not the single bond, adjacent two selected from R¹⁴¹ to R¹⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R⁸¹ to R⁸⁵ which are not the single bond, adjacent two selected from R⁴¹ to R⁴⁶ which are not the single bond, adjacent two selected from R⁵¹ to R⁵⁵, and adjacent two selected from R⁷¹ to R⁸⁰ are each independently not bonded to each other, and therefore do not form a ring structure, and a benzene ring A1 and a benzene ring B1, the benzene ring A1 and a benzene ring C1, the benzene ring B1 and the benzene ring C1, a benzene ring A12 and a benzene ring B12, and a benzene ring A3 and a benzene ring B3 are not crosslinked.

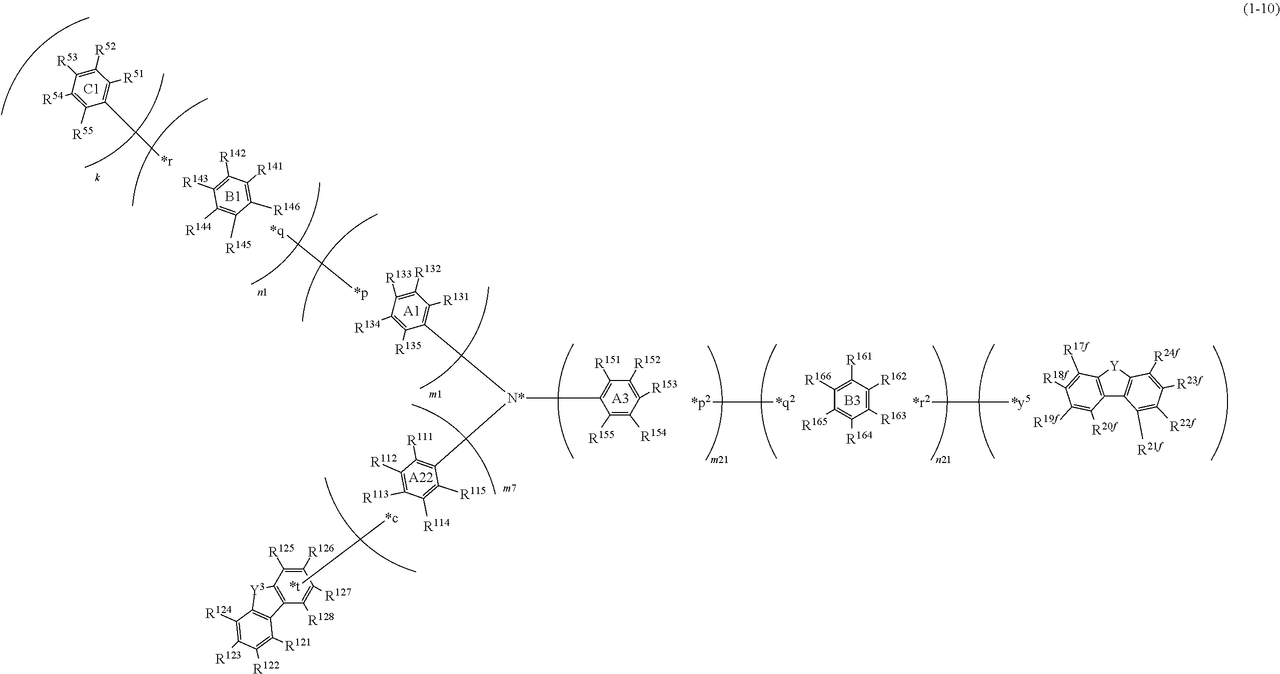

In the formula (1-10), Y and R^{17f} to R^{24f} are as described above.

In the formula (1-10), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-10), R¹³¹ to R¹³⁵, R¹⁴¹ to R¹⁴⁶, R¹¹¹ to R¹¹⁵, R⁵¹ to R⁵⁵, and R¹²¹ to R¹²⁸ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R¹³¹ to R¹³⁵ is a single bond bonded to *p,
one selected from R¹⁴¹ to R¹⁴⁶ is a single bond bonded to *q, and another one or two selected from R¹⁴¹ to R¹⁴⁶ are a single bond bonded to *r,
one selected from R¹¹¹ to R¹¹⁵ is a single bond bonded to *c, and
one selected from R¹²¹ to R¹²⁸ is a single bond bonded to *t.

In the formula (1-10),
m1 and m21 are each independently 0 or 1,
n1 and n21 are each independently 0 or 1,
   when m1 is 0 and n1 is 0, *r is bonded to the central nitrogen atom N*
   when m1 is 0 and n1 is 1, *p is bonded to the central nitrogen atom N*
   when m1 is 1 and n1 is 0, one selected from R¹³¹ to R¹³⁵ is a single bond bonded to *r,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r², and
k is 1 or 2.

In the formula (1-10), m7 is 0 or 1, and *c is bonded to the central nitrogen atom N* when m7 is 0.

In the formula (1-10), Y³ represents an oxygen atom, a sulfur atom, or CR^{c}R^{d}, and
R^{e} and R^{d} each independently represent a substituted or unsubstituted alkyl group having 1 to 50 ring carbon atoms or a substituted or unsubstituted aryl group having 6 to 50 ring carbon atoms. When both R^{e} and R^{d} are aryl groups, R^{e} and R^{d} may be bonded to each other to form a spiro ring.

In the formula (1-10), adjacent two selected from R¹³¹ to R¹³⁵ which are not the single bond, adjacent two selected from R¹⁴¹ to R¹⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R¹¹¹ to R¹¹⁵ which are not the single bond, adjacent two selected from R⁵¹ to R⁵⁵, and adjacent two selected from R¹²¹ to R¹²⁴ and R¹²⁵ to R¹²⁸ are each independently not bonded to each other, and therefore do not form a ring structure, and a benzene ring A1 and a benzene ring B1, the benzene ring A1 and a benzene ring C1, the benzene ring B1 and the benzene ring C1, and a benzene ring A3 and a benzene ring B3 are not crosslinked.

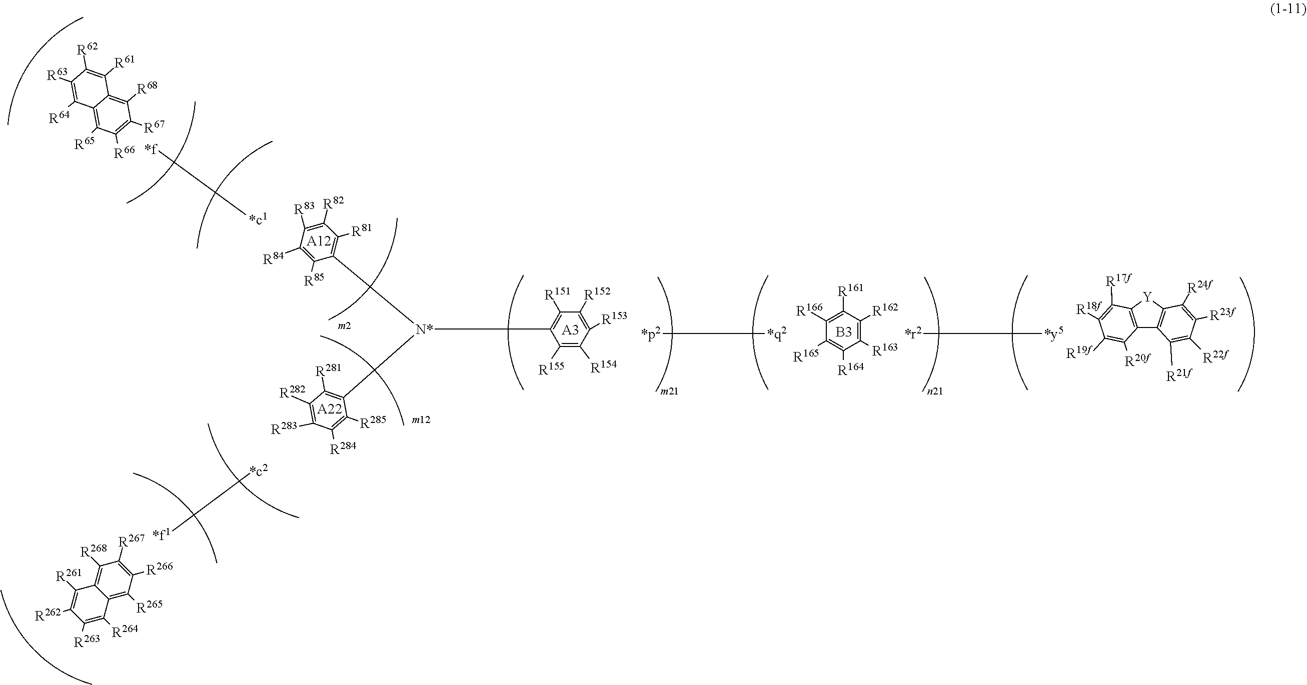

In the formula (1-11), Y and R^{17f} to R^{24f} are as described above.

In the formula (1-11), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-11), R⁸¹ to R⁸⁵, R²⁸¹ to R²⁸⁵, R⁶¹ to R⁶⁸, and R²⁶¹ to R²⁶⁸ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R⁸¹ to R⁸⁵ is a single bond bonded to *c¹,
one selected from R²⁸¹ to R²⁸⁵ is a single bond bonded to *c²,
one selected from R⁶¹ to R⁶⁸ is a single bond bonded to *f, and
one selected from R²⁶¹ to R²⁶⁸ is a single bond bonded to *f¹.

In the formula (1-11),
m21 is 0 or 1, and
n21 is 0 or 1,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r².

In the formula (1-11), m2 is 0 or 1, and *c¹ is bonded to the central nitrogen atom N* when m2 is 0; m12 is 0 or 1, and *c² is bonded to the central nitrogen atom N* when m12 is 0.

In the formula (1-11), adjacent two selected from R⁸¹ to R⁸⁵ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R²⁸¹ to R²⁸⁵ which are not the single bond, adjacent two selected from R⁶¹ to R⁶⁸, and adjacent two selected from R²⁶¹ to R²⁶⁸ are each independently not bonded to each other, and therefore do not form a ring structure, and a benzene ring A3 and a benzene ring B3 are not crosslinked.

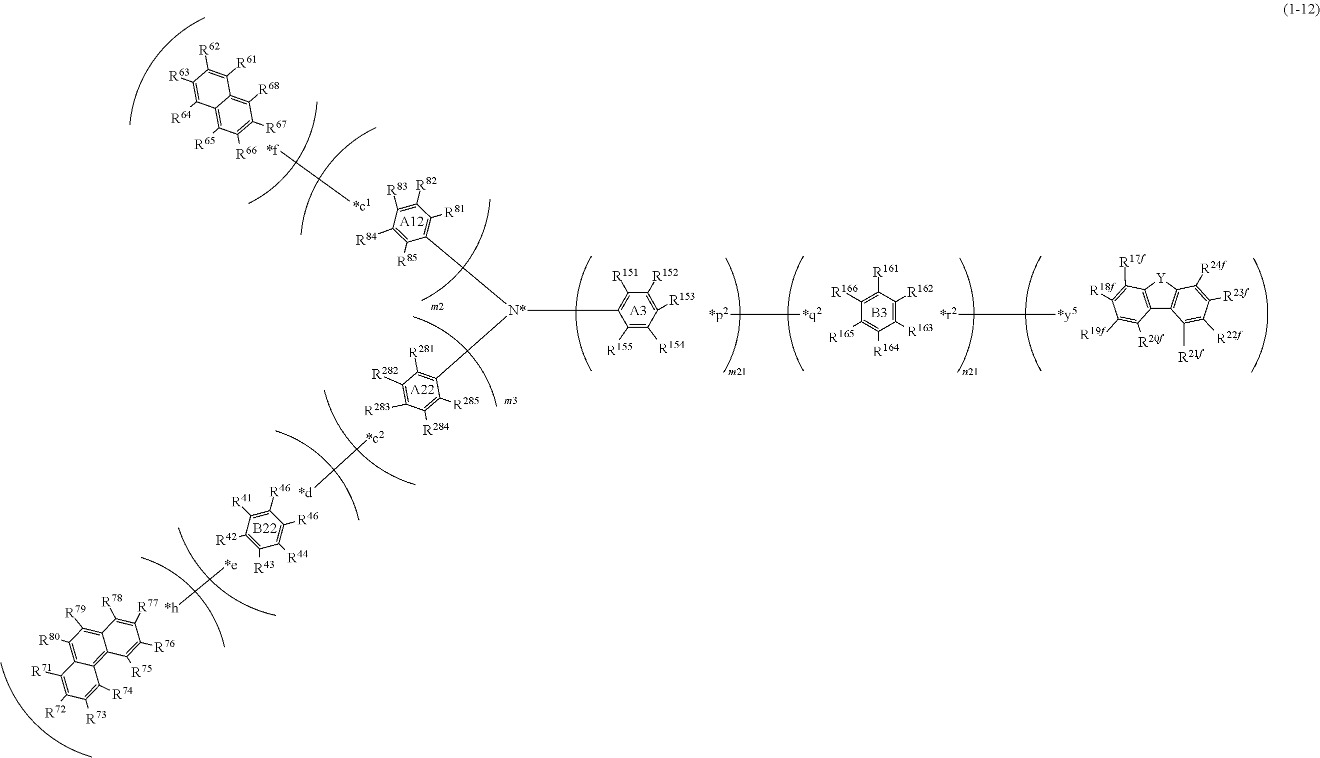

In the formula (1-12), Y and R^{17f} to R^{24f} are as described above.

In the formula (1-12), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-12), R⁸¹ to R⁸⁵, R²⁸¹ to R²⁸⁵, R⁴¹ to R⁴⁶, R⁶¹ to R⁶⁸, and R⁷¹ to R⁸⁰ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R⁸¹ to R⁸⁵ is a single bond bonded to *c¹,
one selected from R²⁸¹ to R²⁸⁵ is a single bond bonded to *c²,
one selected from R⁴¹ to R⁴⁶ is a single bond bonded to *d, and another one selected from R⁴¹ to R⁴⁶ is a single bond bonded to *e,
one selected from R⁶¹ to R⁶⁸ is a single bond bonded to *f, and
one selected from R⁷¹ to R⁸⁰ is a single bond bonded to *h.

In the formula (1-12),
m21 and m3 are each independently 0 or 1, and
n21 and n3 are each independently 0 or 1,
   when m21 is 0 and n21 is 0, *r² is bonded to the central nitrogen atom N*,
   when m21 is 0 and n21 is 1, *p² is bonded to the central nitrogen atom N*,
   when m21 is 1 and n21 is 0, one selected from R¹⁵¹ to R¹⁵⁵ is a single bond bonded to *r²,
   when m3 is 0 and n3 is 0, *e is bonded to the central nitrogen atom N* when m3 is 0 and n3 is 1, *c² is bonded to the central nitrogen atom N*, and
   when m3 is 1 and n3 is 0, one selected from R²⁸¹ to R²⁸⁵ is a single bond bonded to *e.

In the formula (1-12), m2 is 0 or 1, and *c¹ is bonded to the central nitrogen atom N* when m2 is 0.

In the formula (1-12), adjacent two selected from R⁸¹ to R⁸⁵ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R²⁸¹ to R²⁸⁵ which are not the single bond, adjacent two selected from R⁴¹ to R⁴⁶ which are not the single bond, adjacent two selected from R⁶¹ to R⁶⁸, and adjacent two selected from R⁷¹ to R⁸⁰ are each independently not bonded to each other, and therefore do not form a ring structure, and a benzene ring A22 and a benzene ring B22, and a benzene ring A3 and a benzene ring B3 are not crosslinked.

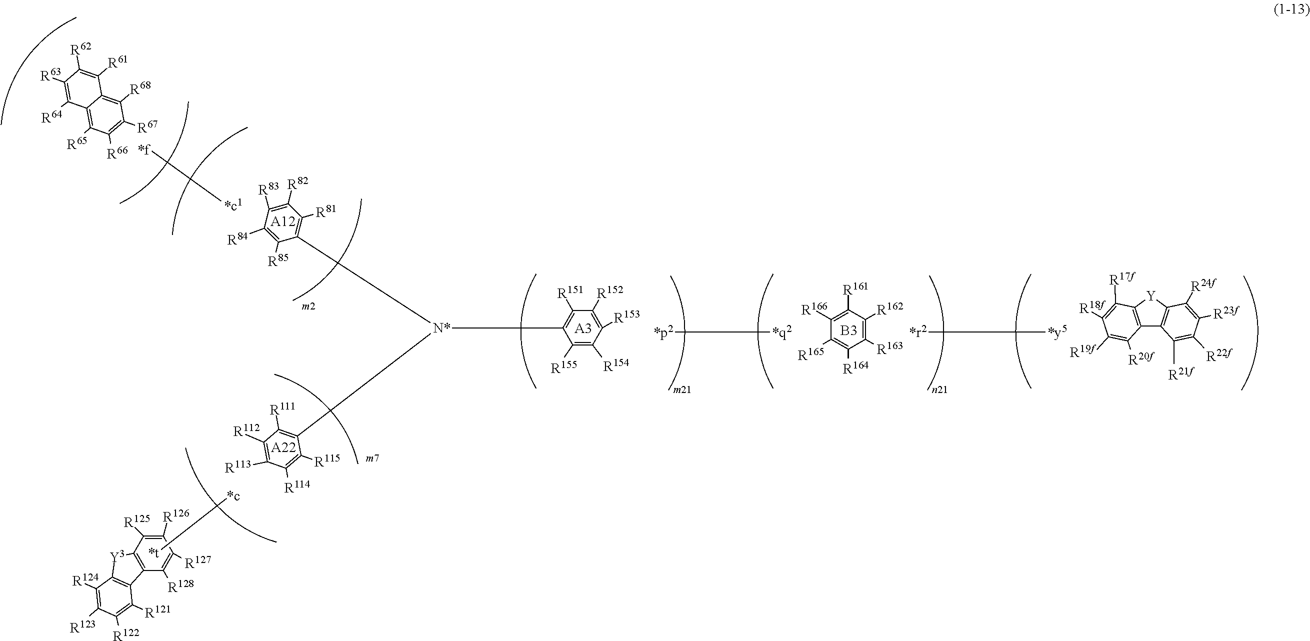

In the formula (1-13), Y and R^{17f} to R^{24f} are as described above.

In the formula (1-13), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-13), R⁸¹ to R⁸⁵, R¹¹¹ to R¹¹⁵, R⁶¹ to R⁶⁸, and R¹²¹ to R¹²⁸ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R⁸¹ to R⁸⁵ is a single bond bonded to *c¹,
one selected from R¹¹¹ to R¹¹⁵ is a single bond bonded to *c²,
one selected from R⁶¹ to R⁶⁵ is a single bond bonded to *f, and
one selected from R¹²¹ to R¹²⁸ is a single bond bonded to *t.

In the formula (1-13),
m21 is 0 or 1, and
n21 is 0 or 1,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r².

In the formula (1-13), m2 is 0 or 1, and *c¹ is bonded to the central nitrogen atom N* when m2 is 0; m7 is 0 or 1, and *c² is bonded to the central nitrogen atom N* when m7 is 0.

In the formula (1-13), Y³ represents an oxygen atom, a sulfur atom, or CR^{c}R^{d}, and
R^{e} and R^{d} each independently represent a substituted or unsubstituted alkyl group having 1 to 50 ring carbon atoms or a substituted or unsubstituted aryl group having 6 to 50 ring carbon atoms. When both R^{e} and R^{d} are aryl groups, R^{e} and R^{d} may be bonded to each other to form a spiro ring.

In the formula (1-13), adjacent two selected from R⁸¹ to R⁸⁵ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R¹¹¹ to R¹¹⁵ which are not the single bond, adjacent two selected from R⁶¹ to R⁶⁸, and adjacent two selected from R¹²¹ to R¹²⁸ are each independently not bonded to each other, and therefore do not form a ring structure, and a benzene ring A3 and a benzene ring B3 are not crosslinked.

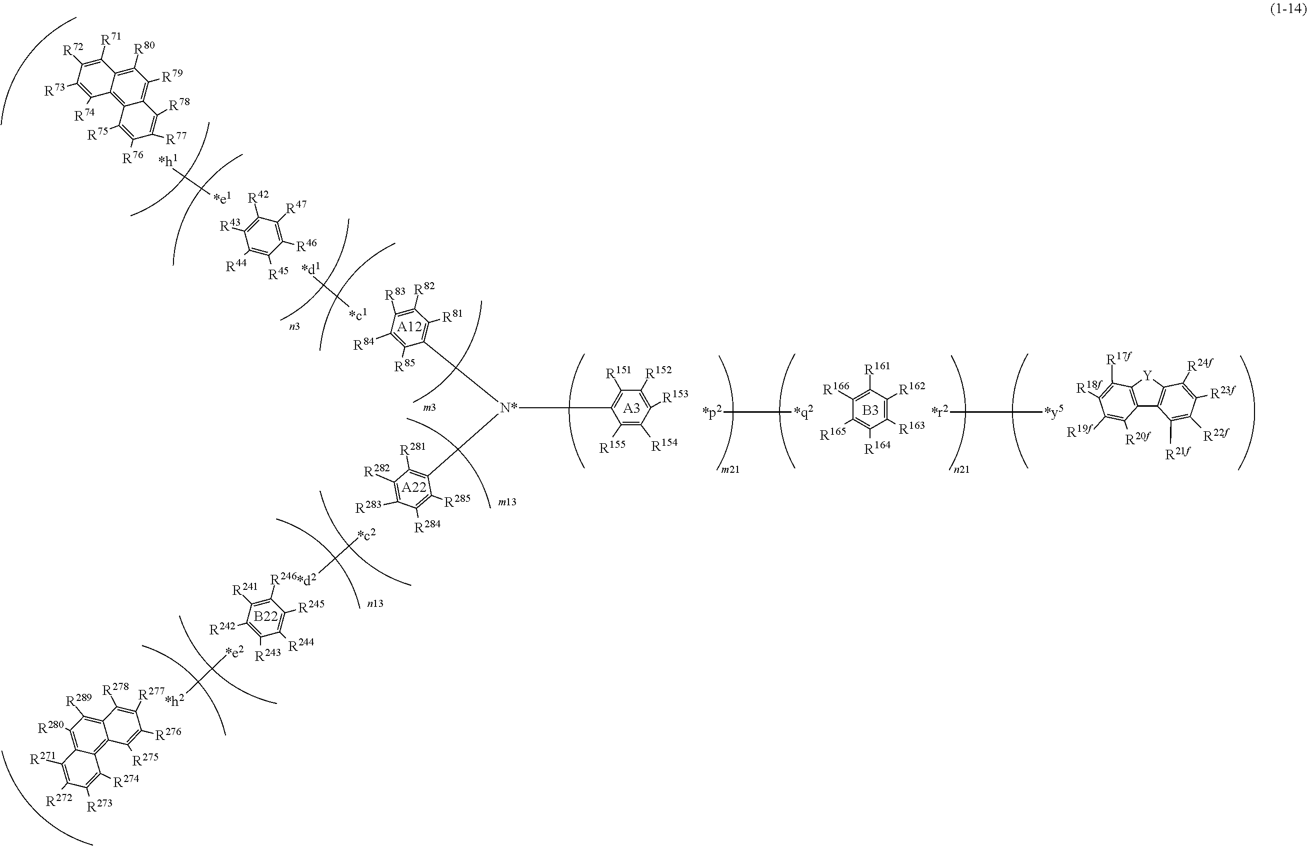

In the formula (1-14), Y and R^{17f} to R^{24f} are as described above.

In the formula (1-14), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-14), R⁸¹ to R⁸⁵, R⁴¹ to R⁴⁶, R²⁸¹ to R²⁸⁵, R²⁴¹ to R²⁴⁶, R⁷¹ to R⁸⁰, and R²⁷¹ to R²⁸⁰ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R⁸¹ to R⁸⁵ is a single bond bonded to *c¹,
one selected from R⁴¹ to R⁴⁶ is a single bond bonded to *d¹, and another one selected from R⁴¹ to R⁴⁶ is a single bond bonded to *e¹,
one selected from R²⁸¹ to R²⁸⁵ is a single bond bonded to *c²,
one selected from R²⁴¹ to R²⁴⁶ is a single bond bonded to *d², and another
one selected from R²⁴¹ to R²⁴⁶ is a single bond bonded to *e²,
one selected from R⁷¹ to R⁸⁰ is a single bond bonded to *h¹, and
one selected from R²⁷¹ to R²⁸⁰ is a single bond bonded to *h².

In the formula (1-14),
m3, m13, and m21 are each independently 0 or 1, and
n3, n13, and n21 are each independently 0 or 1,
   when m3 is 0 and n3 is 0, *e¹ is bonded to the central nitrogen atom N*
   when m3 is 0 and n3 is 1, *c¹ is bonded to the central nitrogen atom N*
   when m3 is 1 and n3 is 0, one selected from R⁸¹ to R⁸⁵ is a single bond bonded to *e¹,
   when m13 is 0 and n13 is 0, *e² is bonded to the central nitrogen atom N*,
   when m13 is 0 and n13 is 1, *c² is bonded to the central nitrogen atom N*,
   when m13 is 1 and n13 is 0, one selected from R²⁸¹ to R²⁸⁵ is a single bond bonded to *e²,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r².

In the formula (1-14), adjacent two selected from R⁸¹ to R⁸⁵ which are not the single bond, adjacent two selected from R⁴¹ to R⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R²⁸¹ to R²⁸⁵ which are not the single bond, adjacent two selected from R²⁴¹ to R²⁴⁶, adjacent two selected from R⁷¹ to R⁸⁰, and adjacent two selected from R²⁷¹ to R²⁸⁰ are each independently not bonded to each other, and therefore do not form a ring structure, and a benzene ring A12 and a benzene ring B12, a benzene ring A22 and a benzene ring B22, and a benzene ring A3 and a benzene ring B3 are not crosslinked.

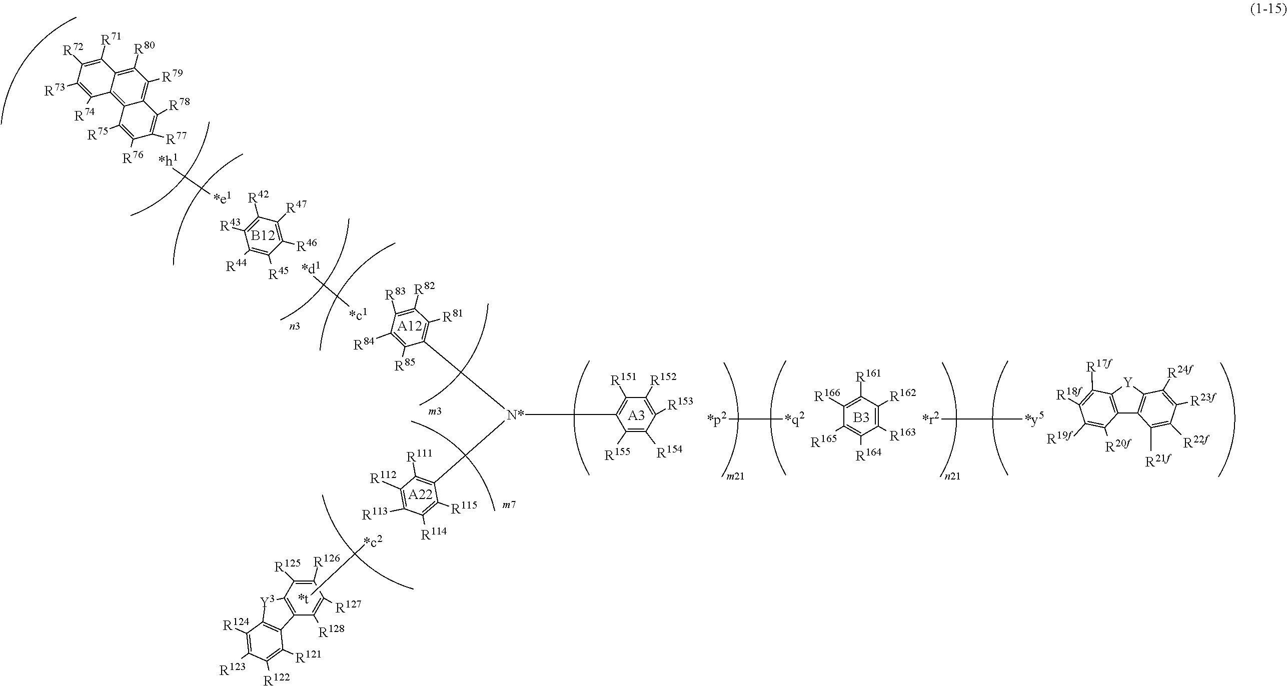

In the formula (1-15), Y and R^{17f} to R^{24f} are as described above.

In the formula (1-15), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-15), R⁸¹ to R⁸⁵, R⁴¹ to R⁴⁶, R¹¹¹ to R¹¹⁵, R⁷¹ to R⁸⁰, and R¹²¹ to R¹²⁸ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R⁸¹ to R⁸⁵ is a single bond bonded to *c¹,
one selected from R⁴¹ to R⁴⁶ is a single bond bonded to *d, and another one selected from R⁴¹ to R⁴⁶ is a single bond bonded to *e,
one selected from R¹¹¹ to R¹¹⁵ is a single bond bonded to *c²,
one selected from R⁷¹ to R⁸⁰ is a single bond bonded to *h, and
one selected from R¹²¹ to R¹²⁸ is a single bond bonded to *t.

In the formula (1-15),
m3 and m21 are each independently 0 or 1, and
n3 and n21 are each independently 0 or 1,
   when m3 is 0 and n3 is 0, *e is bonded to the central nitrogen atom N*
   when m3 is 0 and n3 is 1, *c¹ is bonded to the central nitrogen atom N*
   when m3 is 1 and n3 is 0, one selected from R⁸¹ to R⁸⁵ is a single bond bonded to *e,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r².

In the formula (1-15), m7 is 0 or 1, and *c² is bonded to the central nitrogen atom N* when m7 is 0.

In the formula (1-15), Y³ represents an oxygen atom, a sulfur atom, or CR^{c}R^{d}, and
R^{e} and R^{d} each independently represent a substituted or unsubstituted alkyl group having 1 to 50 ring carbon atoms or a substituted or unsubstituted aryl group having 6 to 50 ring carbon atoms. The case where both R^{e} and R^{d} are substituted or unsubstituted aryl groups each having 6 to 50 ring carbon atoms is excluded.

In the formula (1-15), adjacent two selected from R⁸¹ to R⁸⁵ which are not the single bond, adjacent two selected from R⁴¹ to R⁴⁶ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R¹¹¹ to R¹¹⁵ which are not the single bond, adjacent two selected from R⁷¹ to R⁸⁰, and adjacent two selected from R¹²¹ to R¹²⁸ are each independently not bonded to each other, and therefore do not form a ring structure, and a benzene ring A12 and a benzene ring B12, and a benzene ring A3 and a benzene ring B3 are not crosslinked.

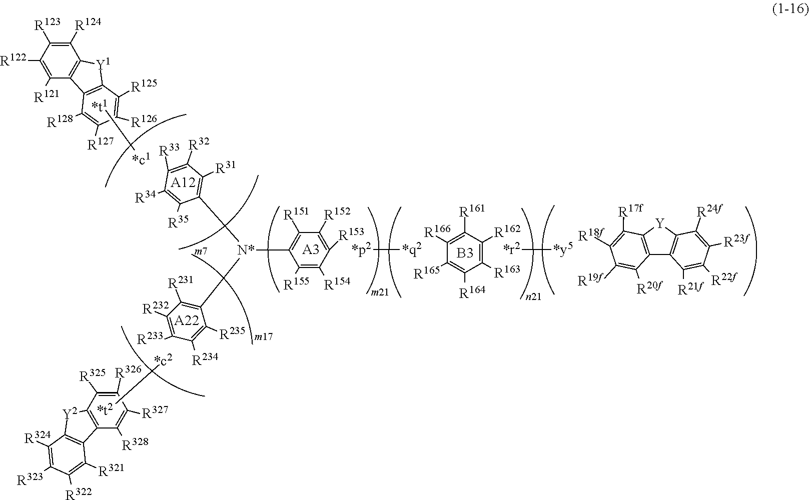

In the formula (1-16), Y and R^{17f} to R^{24f} are as described above.

In the formula (1-16), R¹⁵¹ to R¹⁵⁵ and R¹⁶¹ to R¹⁶⁶ each independently represent a hydrogen atom, a substituted or unsubstituted alkyl group having 1 to 50 carbon atoms, or a substituted or unsubstituted aryl group having 6 to 50 ring carbon atoms, provided that
one selected from R¹⁵¹ to R¹⁵⁵ is a single bond bonded to *p², and
one selected from R¹⁶¹ to R¹⁶⁶ is a single bond bonded to *q², and another
one selected from R¹⁶¹ to R¹⁶⁶ is a single bond bonded to *r².

In the formula (1-16), R³¹ to R³⁵, R²³¹ to R²³⁵, R¹²¹ to R¹²⁸, and R³²¹ to R³²⁸ are each independently the same as R¹³¹ to R¹³⁵, R¹⁴¹ to R¹⁴⁶, R¹⁷¹ to R¹⁷⁵, R¹⁸¹ to R¹⁸⁶, R⁵¹ to R⁵⁵, and R¹⁹¹ to R¹⁹⁵ in the formula (1-7) described above, and a preferred aspect is also the same, provided that
one selected from R³¹ to R³⁵ is a single bond bonded to *c¹,
one selected from R²³¹ to R²³⁵ is a single bond bonded to *c²,
one selected from R¹²¹ to R¹²⁸ is a single bond bonded to *t¹, and
one selected from R³²¹ to R³²⁸ is a single bond bonded to *t².

In the formula (1-16),
m21 is each independently 0 or 1, and
n21 is each independently 0 or 1,
   when m21 is 0 and n21 is 0, *r² is bonded to the central nitrogen atom N*,
   when m21 is 0 and n21 is 1, *p² is bonded to the central nitrogen atom N*, and
   when m21 is 1 and n21 is 0, one selected from R¹⁵¹ to R¹⁵⁵ is a single bond bonded to *r².

In the formula (1-16), m7 is 0 or 1, and *c¹ is bonded to the central nitrogen atom N* when m7 is 0; m17 is 0 or 1, and *c² is bonded to the central nitrogen atom N* when m17 is 0.

In the formula (1-16), Y¹ and Y² each independently represent an oxygen atom, a sulfur atom, or CR^{c}R^{d}, and
R^{e} and R^{d} each independently represent a substituted or unsubstituted alkyl group having 1 to 50 ring carbon atoms or a substituted or unsubstituted aryl group having 6 to 50 ring carbon atoms. When both R^{e} and R^{d} are aryl groups, R^{e} and R^{d} may be bonded to each other to form a spiro ring.

In the formula (1-16), adjacent two selected from R³¹ to R³⁵ which are not the single bond, adjacent two selected from R¹⁵¹ to R¹⁵⁵ which are not the single bond, adjacent two selected from R¹⁶¹ to R¹⁶⁶ which are not the single bond, adjacent two selected from R²³¹ to R²³⁵ which are not the single bond, adjacent two selected from R¹²¹ to R¹²⁸, and adjacent two selected from R³²¹ to R³²⁸ are each independently not bonded to each other, and therefore do not form a ring structure, and a benzene ring A3 and a benzene ring B3 are not crosslinked.

### (Deuterium Atom in Compound (1))

As described above, the “hydrogen atom” used in the description herein encompasses a protium atom, a deuterium atom, and a tritium atom. Therefore, the compound (1) may have a naturally-derived deuterium atom.

A deuterium atom may be intentionally introduced into the compound (1) by using a compound obtained by partially or wholly deuterating a raw material compound. Therefore, in one aspect of the present invention, the compound (1) has at least one deuterium atom. That is, the compound (1) may be a compound represented by the formula (1) in which at least one of hydrogen atoms in the compound is a deuterium atom.

Here, examples of the partially or wholly deuterated raw material compound include a compound forming a terminal portion (Ar¹, Ar², Ar³) of the formula (1) and a compound forming a linker portion (L¹, L², L³) connecting the central nitrogen atom and the terminal portion of the formula (1).

A deuteration ratio of the compound (1) depends on a deuteration ratio of the raw material compound to be used. Even when a raw material having a predetermined deuteration ratio is used, a naturally-derived protium isotope may still be contained at a certain ratio. Accordingly an aspect of the deuteration ratio of the compound (1) shown below includes a ratio obtained by simply counting the number of deuterium atoms represented in a chemical formula and a ratio obtained by taking into account a trace amount of naturally-derived isotopes.

The deuteration ratio of the compound (1) contained in the first compound is 1% or more, preferably 3% or more, more preferably 5% or more, even more preferably 10% or more, and still more preferably 50% or more. In other words, the first compound contains 1 mass % or more, preferably 3 mass % or more, more preferably 5 mass % or more, even more preferably 10 mass % or more, and still more preferably 50 mass % or more of the compound having one or more of deuterium atoms in the compound represented by the formula (1) (the compound (1)).

The compound (1) may be a mixture containing a deuterated compound and a non-deuterated compound, or a mixture of two or more compounds having different deuteration ratios. For example, the compound (1) may be a mixture of the following compound A1 and compound A2, a mixture of the following compound D1 and compound D2, or a mixture of the following compound H1 and compound H2. In these cases, the first compound may contain the following compounds A1 and A2, the following compounds D1 and D2, or the following compounds H1 and H2.

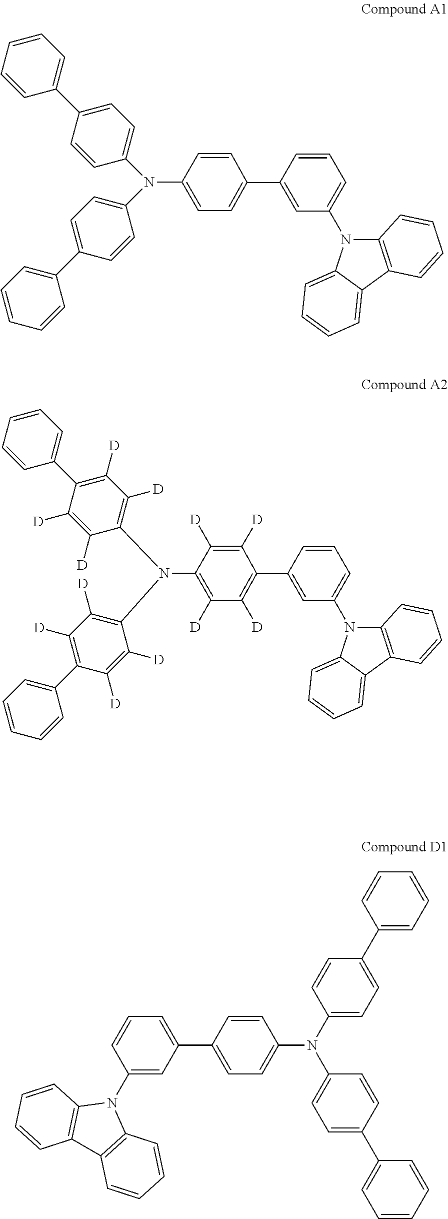 [SMILES: c1ccc(-c2ccc(N(c3ccc(-c4ccccc4)cc3)c3ccc(-c4cccc(-n5c6ccccc6c6ccccc65)c4)cc3)cc2)cc1 ; [2H]c1c([2H])c(N(c2c([2H])c([2H])c(-c3ccccc3)c([2H])c2[2H])c2c([2H])c([2H])c(-c3cccc(-n4c5ccccc5c5ccccc54)c3)c([2H])c2[2H])c([2H])c([2H])c1-c1ccccc1 ; c1ccc(-c2ccc(N(c3ccc(-c4ccccc4)cc3)c3ccc(-c4cccc(-n5c6ccccc6c6ccccc65)c4)cc3)cc2)cc1]

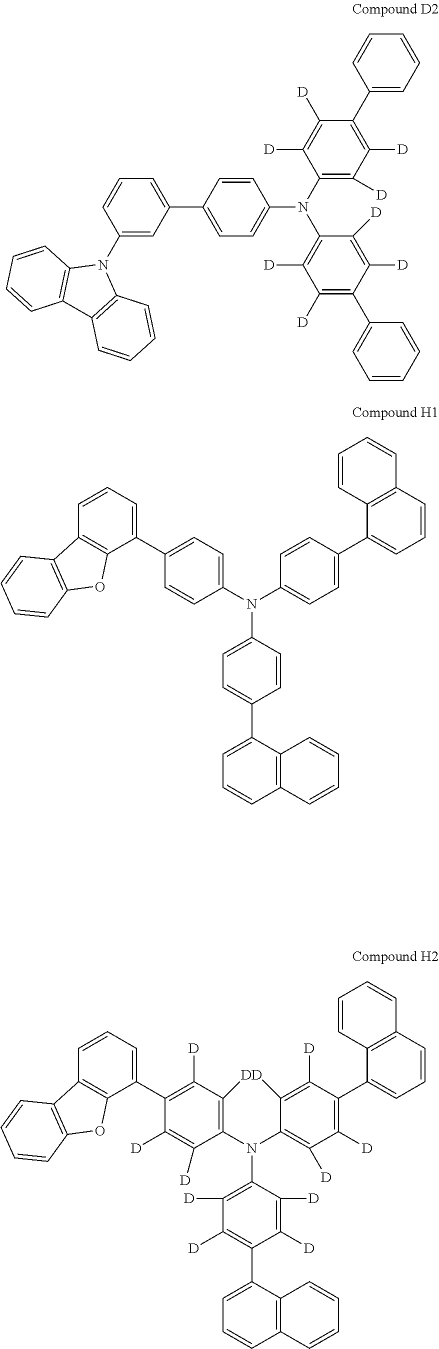 [SMILES: [2H]c1c([2H])c(N(c2ccc(-c3cccc(-n4c5ccccc5c5ccccc54)c3)cc2)c2c([2H])c([2H])c(-c3ccccc3)c([2H])c2[2H])c([2H])c([2H])c1-c1ccccc1 ; c1ccc2c(-c3ccc(N(c4ccc(-c5cccc6ccccc56)cc4)c4ccc(-c5cccc6c5oc5ccccc56)cc4)cc3)cccc2c1 ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1cccc2ccccc12]

A deuteration ratio of such a mixture is preferably 1% or more, more preferably 3% or more, even more preferably 5% or more, still more preferably 10% or more, and even still more preferably 50% or more, and is less than 100%.

A ratio of the number of deuterium atoms to the total number of hydrogen atoms in the compound (1) is preferably 1% or more, more preferably 3% or more, even more preferably 5% or more, and still more preferably 10% or more, and is 100% or less.

It is preferred that, in the compound represented by the formula (1) (the compound (1)) contained in the first compound, at least one of a hydrogen atom directly bonded to the arylene group represented by L¹, a hydrogen atom directly bonded to the divalent heterocyclic group represented by L¹, a hydrogen atom directly bonded to the arylene group represented by L², a hydrogen atom directly bonded to the divalent heterocyclic group represented by L², a hydrogen atom directly bonded to the arylene group represented by L³, a hydrogen atom directly bonded to the divalent heterocyclic group represented by L³, a hydrogen atom directly bonded to the aryl group represented by Ar¹, a hydrogen atom directly bonded to the monovalent heterocyclic group represented by Ar¹, a hydrogen atom directly bonded to the aryl group represented by Ar², a hydrogen atom directly bonded to the monovalent heterocyclic group represented by Ar², a hydrogen atom directly bonded to the aryl group represented by Ar³, and a hydrogen atom directly bonded to the monovalent heterocyclic group represented by Ar³ is a deuterium atom.

That is, in the following formula (1-17), the sum of a, b, c, d, e, and f is preferably 1 or more.

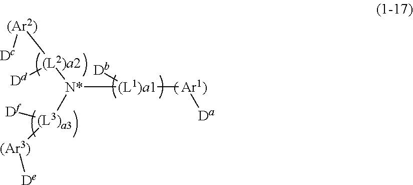

In the formula (1-17), “D^{a}” indicates that a of the hydrogen atoms directly bonded to the aryl group or the monovalent heterocyclic group represented by Ar¹ are deuterium atoms. “D^{b}” indicates that b of the hydrogen atoms represented by hydrogen atoms directly bonded to 1 to 3 arylene groups or divalent heterocyclic groups represented by -((L¹)a1)- are deuterium atoms. “D^{c}” indicates that c of the hydrogen atoms directly bonded to the aryl group or the monovalent heterocyclic group represented by Ar² are deuterium atoms. “D^{d}” indicates that d of the hydrogen atoms directly bonded to 1 to 3 arylene groups or divalent heterocyclic groups represented by -((L²)a2)- are deuterium atoms. “D^{e}” indicates that e of the hydrogen atoms directly bonded to the aryl group or the monovalent heterocyclic group represented by Ar³ are deuterium atoms, and “D^{f}” indicates that f of the hydrogen atoms directly bonded to 1 to 3 arylene groups or divalent heterocyclic groups represented by -((L³)a3)- are deuterium atoms.

Further, it is more preferred that at least one deuterium atom in the compound (1) contained in the first compound is at least one of the hydrogen atom directly bonded to the arylene group represented by L¹, the hydrogen atom directly bonded to the divalent heterocyclic group represented by L¹, the hydrogen atom directly bonded to the arylene group represented by L², the hydrogen atom directly bonded to the divalent heterocyclic group represented by L², the hydrogen atom directly bonded to the arylene group represented by L³, the hydrogen atom directly bonded to the divalent heterocyclic group represented by L³, the hydrogen atom directly bonded to the aryl group represented by Ar¹, the hydrogen atom directly bonded to the monovalent heterocyclic group represented by Ar¹, the hydrogen atom directly bonded to the aryl group represented by Ar², the hydrogen atom directly bonded to the monovalent heterocyclic group represented by Ar², the hydrogen atom directly bonded to the aryl group represented by Ar³, and the hydrogen atom directly bonded to the monovalent heterocyclic group represented by Ar³ (that is, in the compound (1), the substituent when Ar¹ to Ar³ and L¹ to L³ are substituted does not have a deuterium atom).

Suitable examples of the compound (1) having a deuterium atom and contained in the first compound include compounds represented by the following formula (1-18a) or (1-18b).

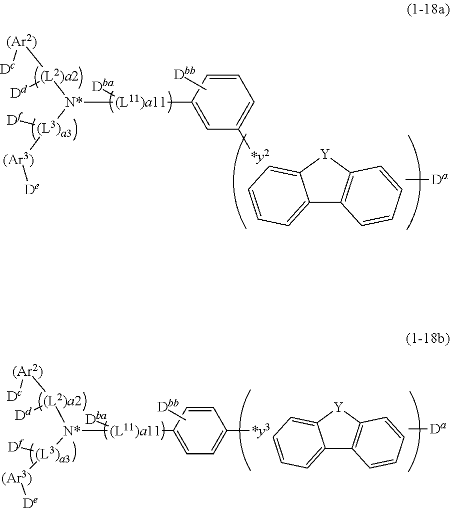

In the formula (1-18a) and the formula (1-18b), “D^{a}”, “D^{c}”, “D^{d}” “D^{e}” and “D” are as defined in the formula (1-17a) and the formula (1-17b). “D^{ba}” indicates that ba of the hydrogen atoms represented by hydrogen atoms directly bonded to 1 to 2 arylene groups or divalent heterocyclic groups represented by -((L¹¹)a11)- are deuterium atoms. “D^{bb}” indicates that bb of R²⁵ to R²⁸ in the formula (1-18a) are deuterium atoms, and bb of R²⁵ to R²⁷ and R²⁹ in the formula (1-18b) are deuterium atoms.

In the formula (1-18a) and the formula (1-18b), Y is as defined in the formula (1-3a) and the formula (1-3b).

In the formula (1-18a) and the formula (1-18b), R is omitted.

In one preferred aspect of the present invention, in the compound represented by the formula (1) (the compound (1)) contained in the first compound, the at least one deuterium atom is at least one of the hydrogen atom directly bonded to the arylene group represented by L¹, the hydrogen atom directly bonded to the divalent heterocyclic group represented by L¹, the hydrogen atom directly bonded to the arylene group represented by L², the hydrogen atom directly bonded to the divalent heterocyclic group represented by L², the hydrogen atom directly bonded to the arylene group represented by L³, and the hydrogen atom directly bonded to the divalent heterocyclic group represented by L³.

That is, in the formula (1-17), the sum of b, d, and f is preferably 1 or more, and in the formula (1-18a) and the formula (1-18b), the sum of ba, bb, d, and f is preferably 1 or more.

In one preferred aspect of the present invention, the first compound contains 10 mass % or more of the compound represented by the formula (1) (the compound (1)) in which at least one of the hydrogen atom directly bonded to the arylene group represented by L¹, the hydrogen atom directly bonded to the divalent heterocyclic group represented by L¹, the hydrogen atom directly bonded to the arylene group represented by L², the hydrogen atom directly bonded to the divalent heterocyclic group represented by L², the hydrogen atom directly bonded to the arylene group represented by L³, and the hydrogen atom directly bonded to the divalent heterocyclic group represented by L³ is a deuterium atom.

A content of the compound having the deuterium atom in the compound (1) contained in the first compound is more preferably 10 mass % or more, and even more preferably 50 mass % or more.

Suitable examples of the compound (1) include compounds represented by the following formulae (1-18-1a) to (1-18-16b).

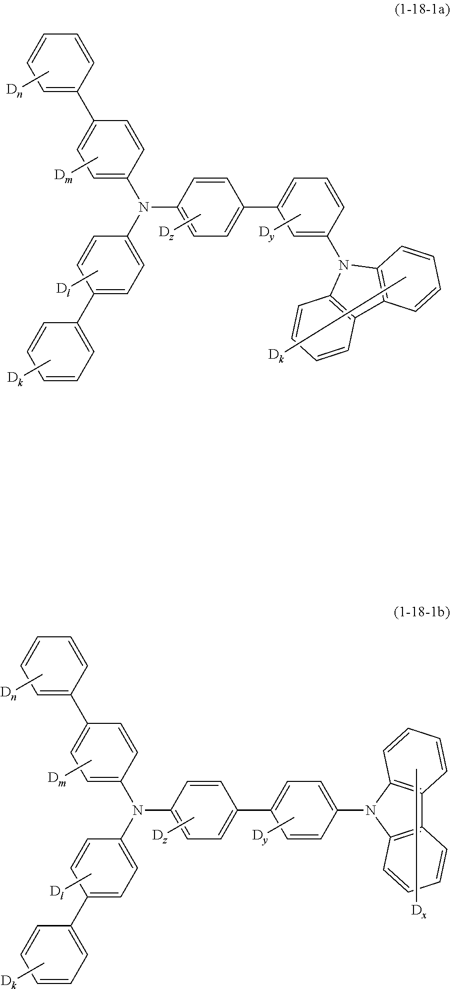

(In the formula (1-18-1a) and the formula (1-18-1b), x+y+z+k+1+m+n=1 to 34, and each R is omitted.)

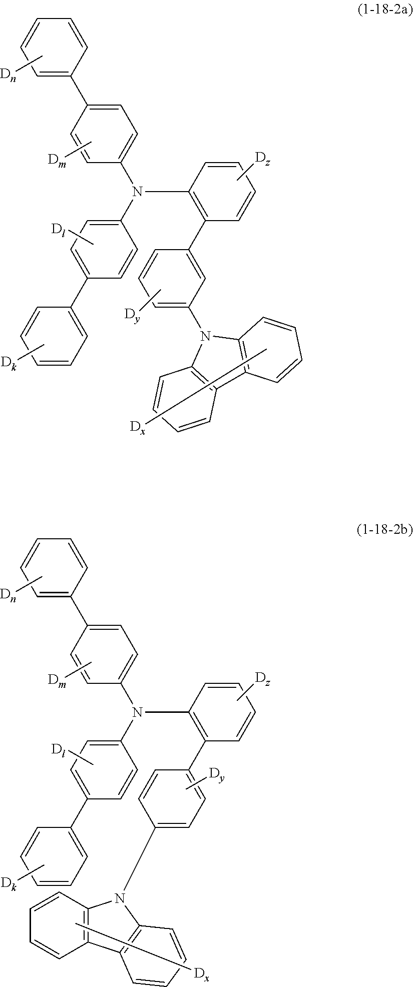

(In the formula (1-18-2a) and the formula (1-18-2b), x+y+z+k+1+m+n=1 to 34, and each R is omitted.)

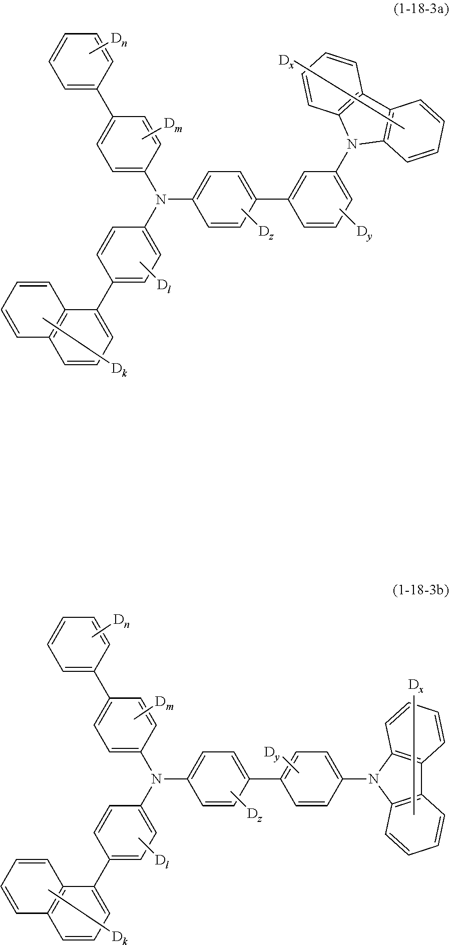

(In the formula (1-18-3a) and the formula (1-18-3b), x+y+z+k+1+m+n=1 to 36, and each R is omitted.)

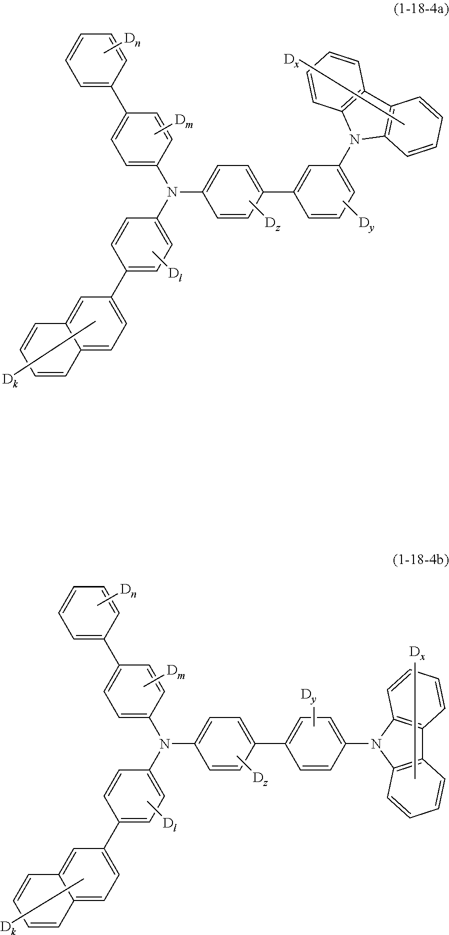

(In the formula (1-18-4a) and the formula (1-18-4b), x+y+z+k+1+m+n=1 to 36, and each R is omitted.)

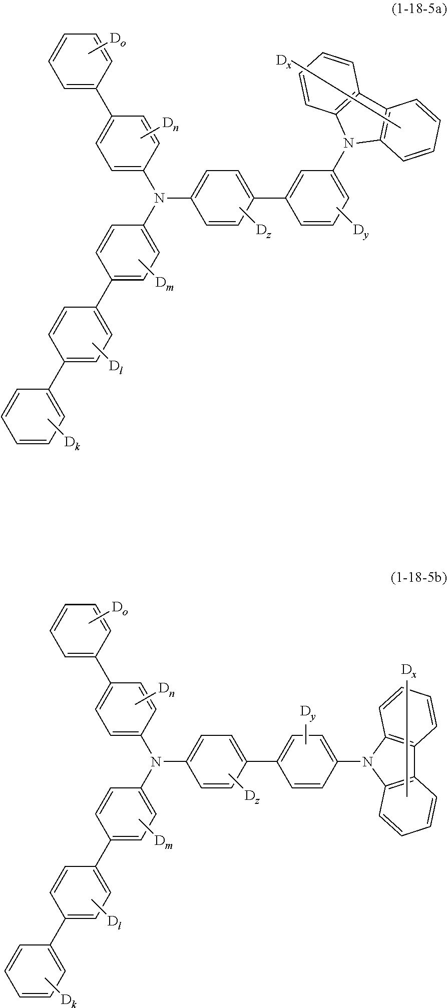

(In the formula (1-18-5a) and the formula (1-18-5b), x+y+z+k+1+m+n+o=1 to 38, and each R is omitted.)

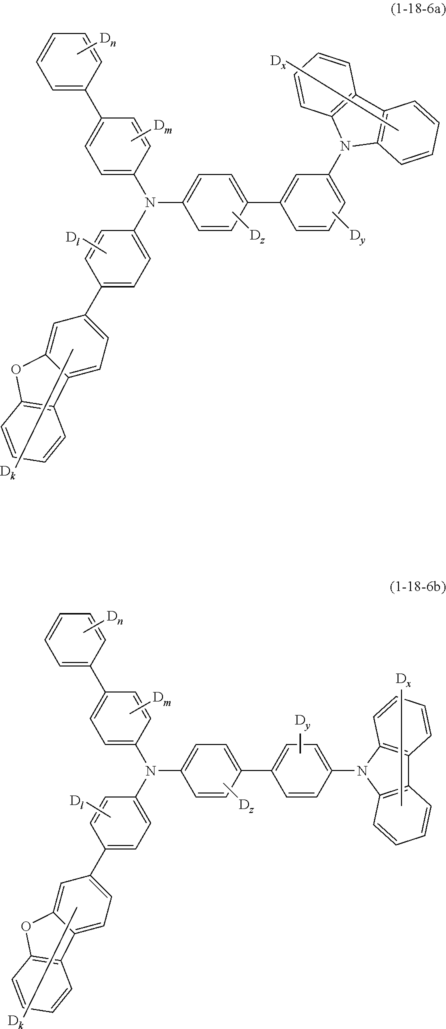

(In the formula (1-18-6a) and the formula (1-18-6b), x+y+z+k+1+m+n=1 to 36, and each R is omitted.)

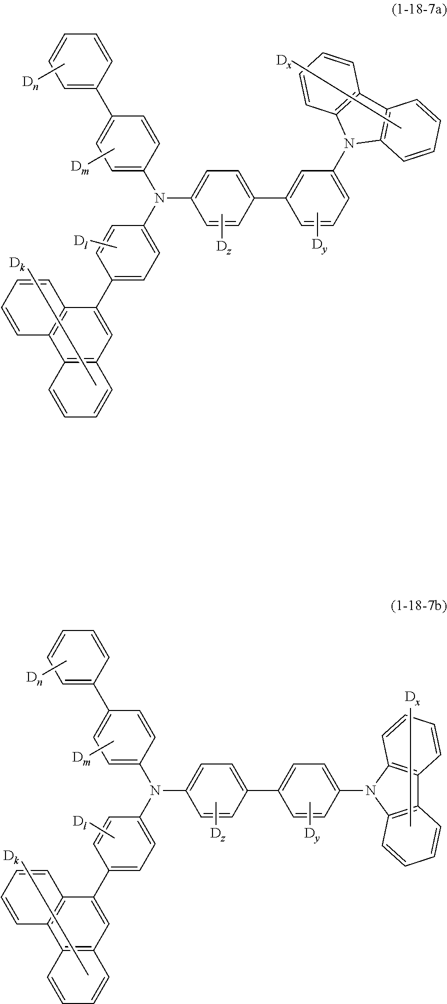

(In the formula (1-18-7a) and the formula (1-18-7b), x+y+z+k+1+m+n=1 to 38, and each R is omitted.)

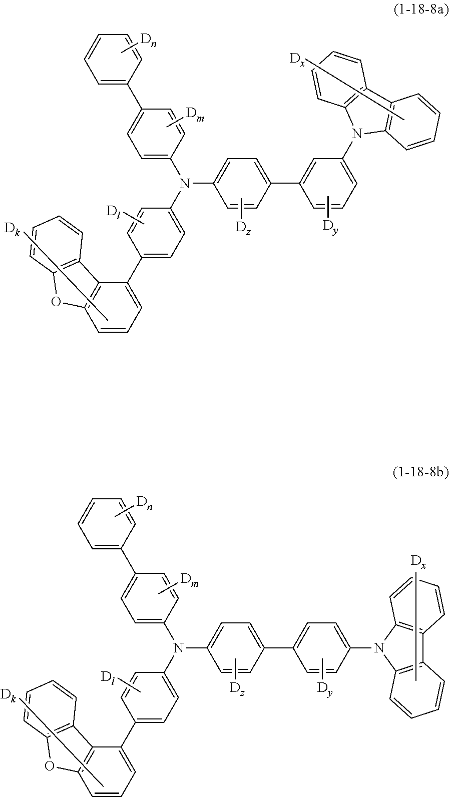

(In the formula (1-18-8a) and the formula (1-18-8b), x+y+z+k+1+m+n=1 to 36, and each R is omitted.)

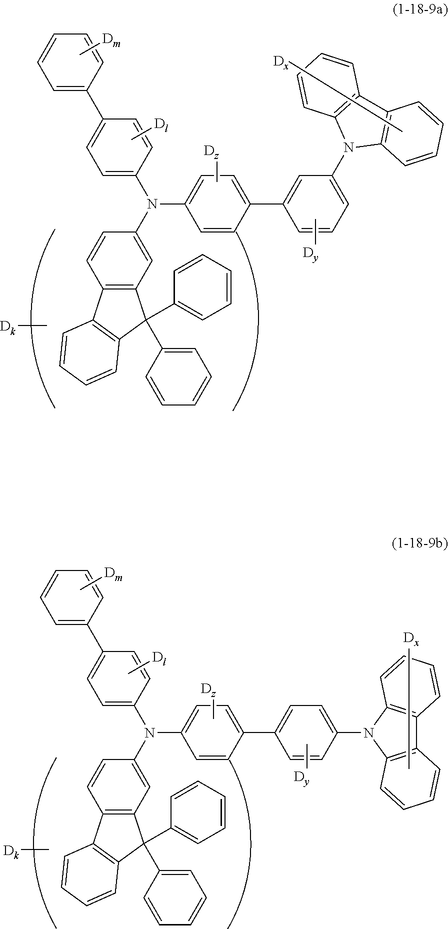

(In the formula (1-18-9a) and the formula (1-18-9b), x+y+z+k+1+m=1 to 43, and each R is omitted.)

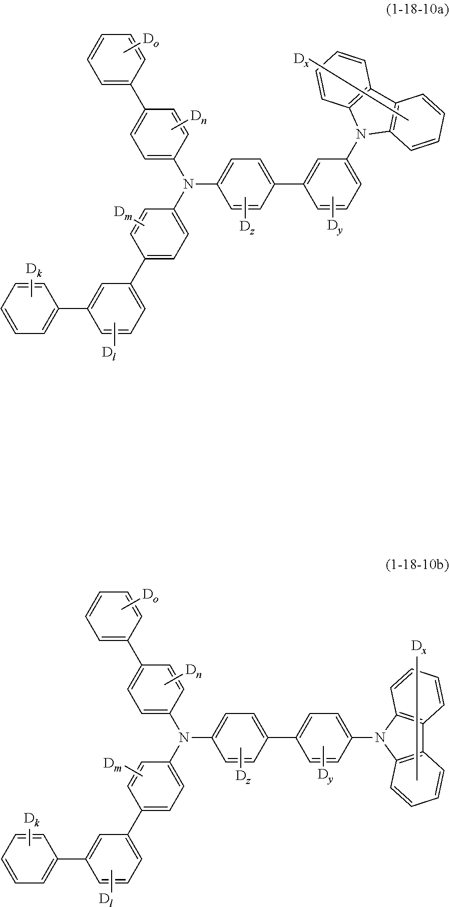

(In the formula (1-18-10a) and the formula (1-18-10b), x+y+z+k+1+m+n+o=1 to 38, and each R is omitted.)

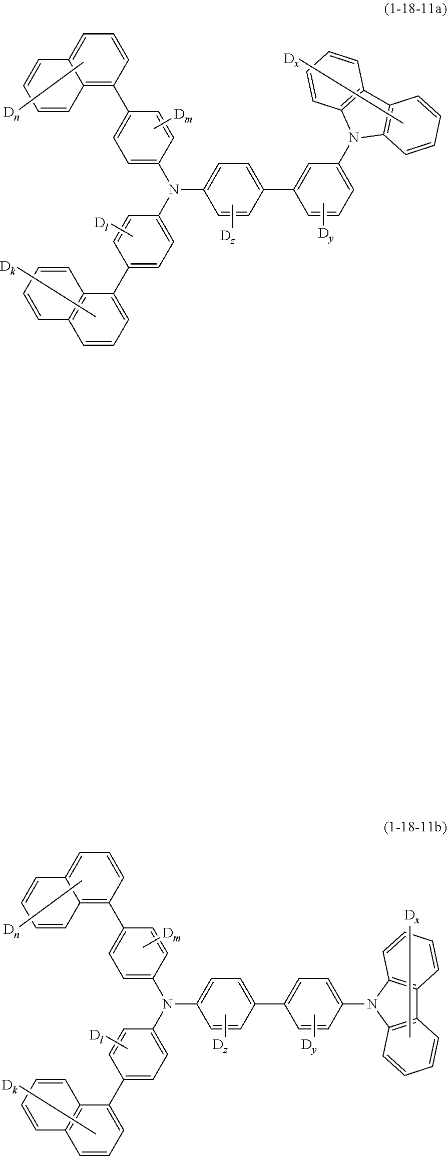

(In the formula (1-18-11a) and the formula (1-18-11b), x+y+z+k+1+m

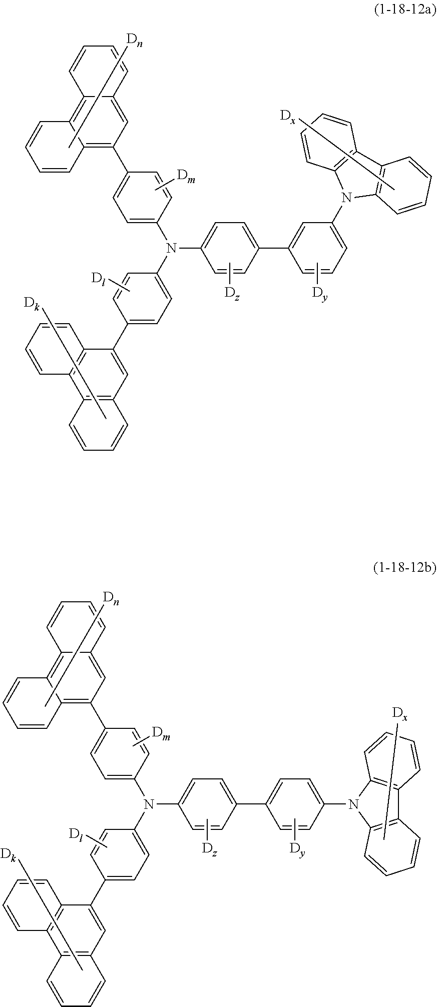

(In the formula (1-18-12a) and the formula (1-18-12b), x+y+z+k+1+m+n=1 to 42, and each R is omitted.)

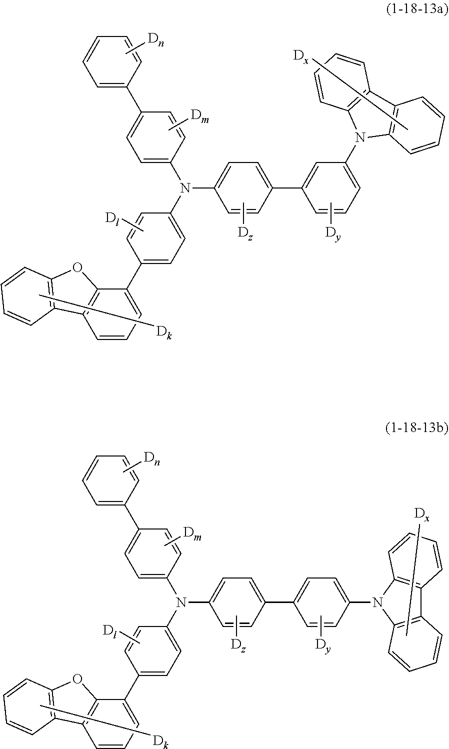

(In the formula (1-18-13a) and the formula (1-18-13b), x+y+z+k+1+m+n=1 to 36, and each R is omitted.)

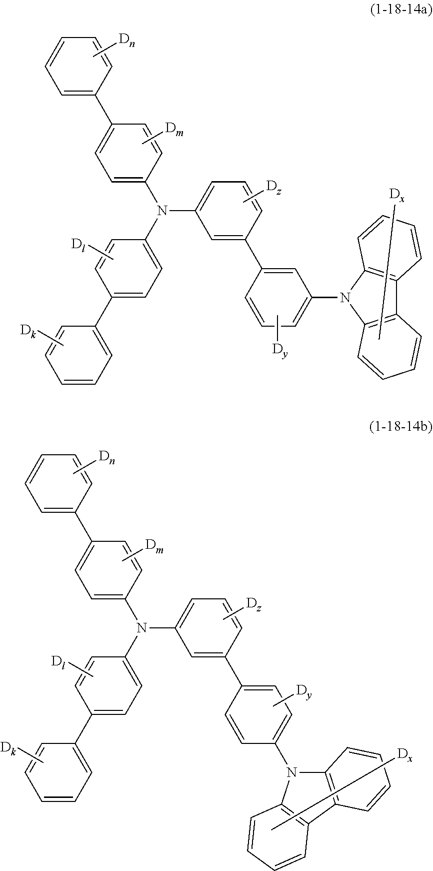

(In the formula (1-18-14a) and the formula (1-18-14b), x+y+z+k+1+m+n=1 to 34, and each R is omitted.)

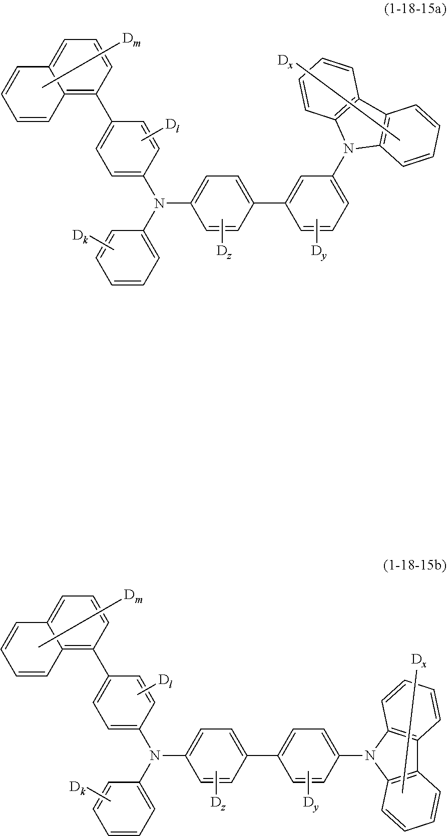

(In the formula (1-18-15a) and the formula (1-18-15b), x+y+z+k+1+m=1 to 32, and each R is omitted.)

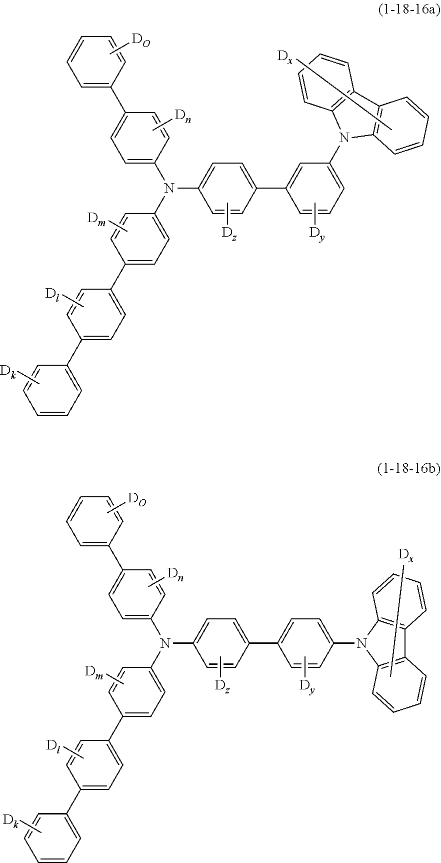

(In the formula (1-18-16a) and the formula (1-18-16b), x+y+z+k+1+m+n+o=1 to 38, and each R is omitted.)

Unless otherwise specified, details of the substituent (any substituent) for the case of “substituted or unsubstituted” included in the definition of each formula are as described in the section “Substituent for ‘Substituted or Unsubstituted’”.

The compound (1) can be easily produced by those skilled in the art with reference to Synthesis Examples below and known synthesis methods.

Specific examples of the compound (1) according to the embodiment of the present invention are shown below. In the following specific examples, “D” represents a deuterium atom. The compound (1) according to the embodiment of the present invention is not limited to these specific examples.

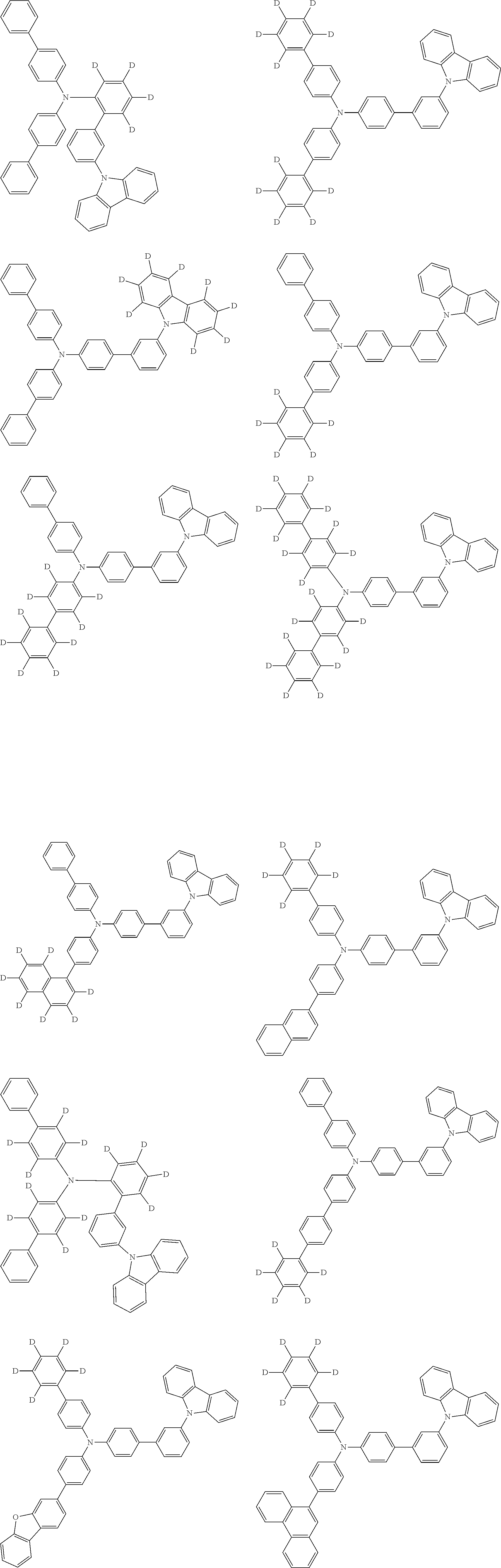

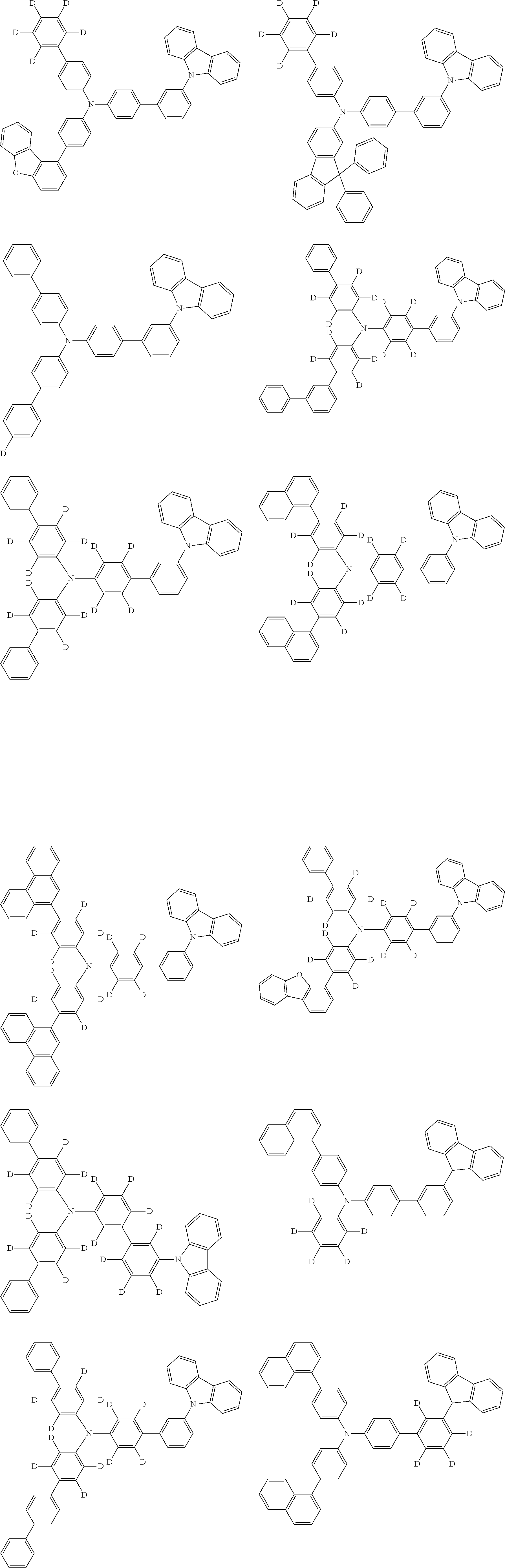

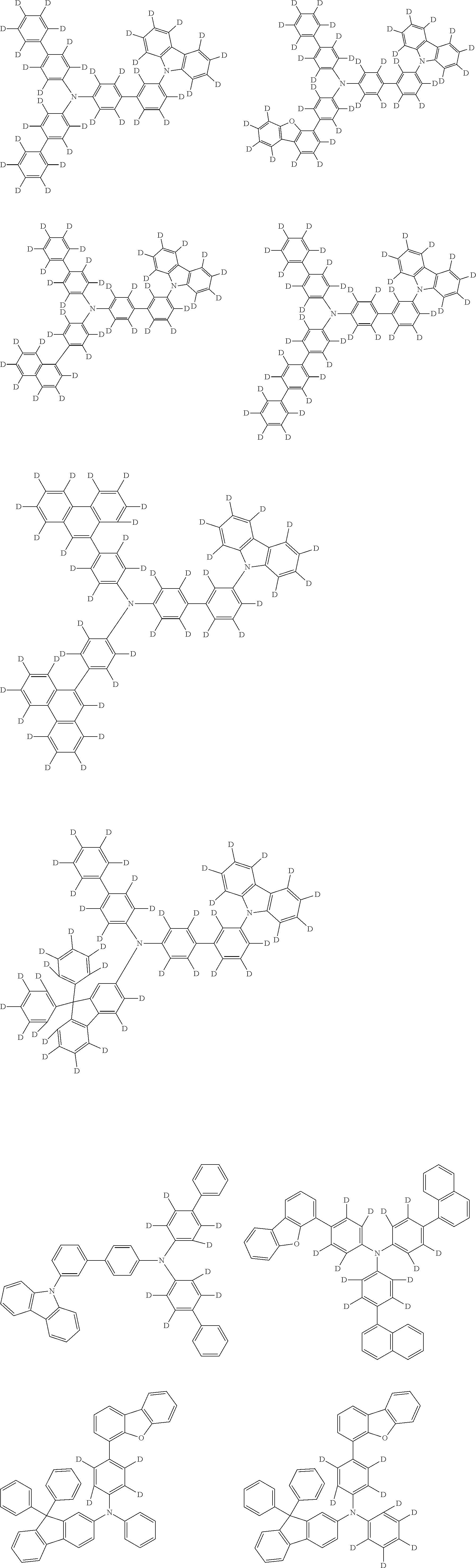

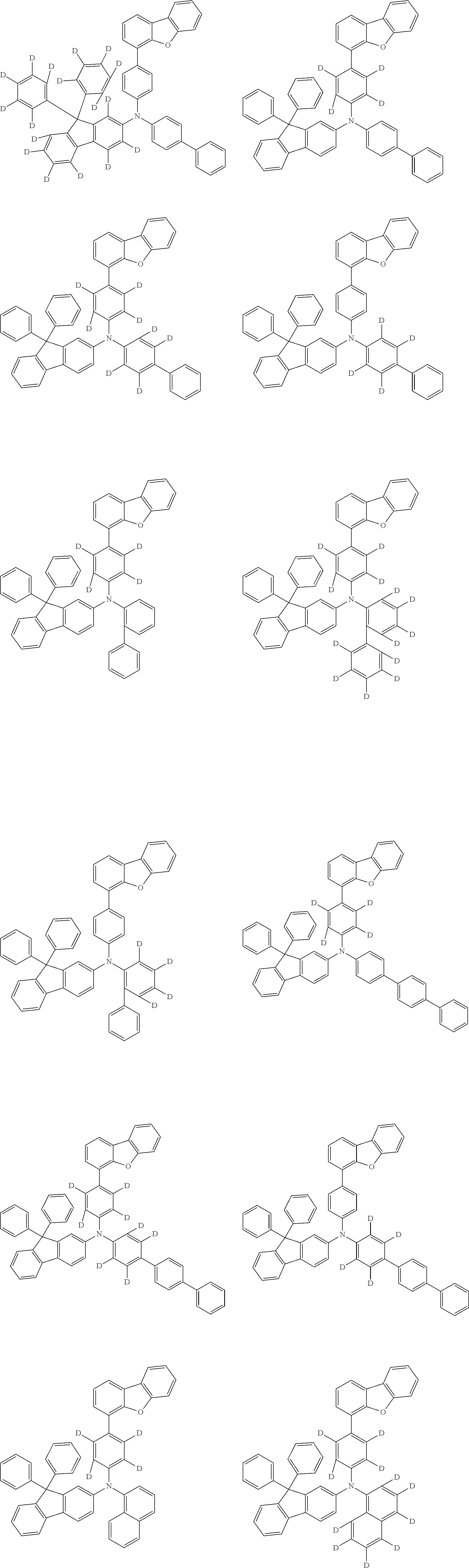

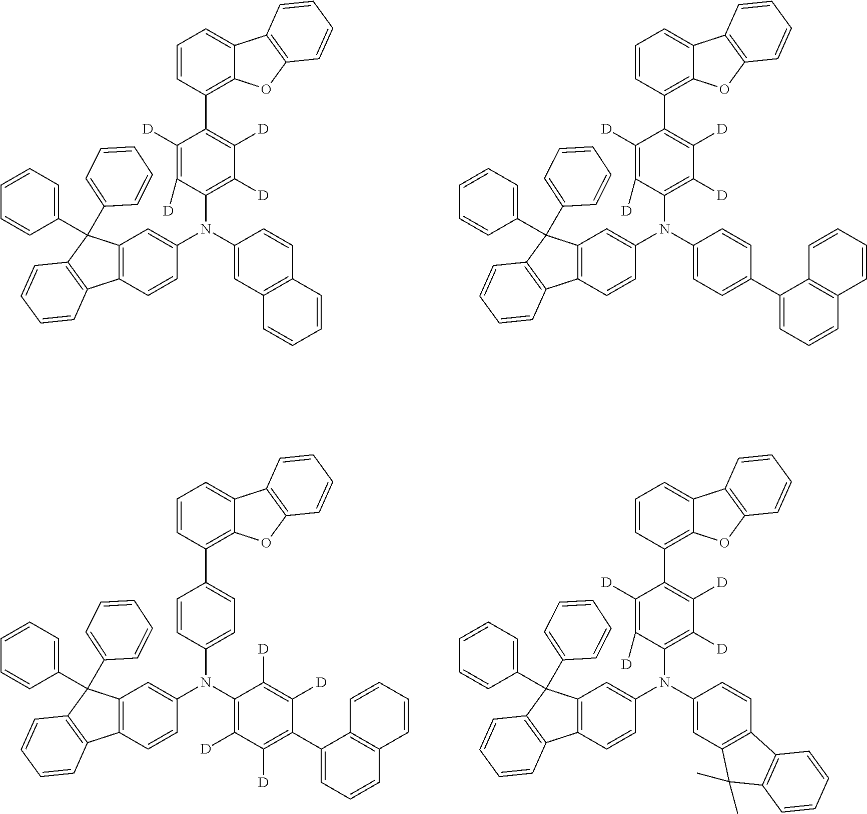 [SMILES: [2H]c1c([2H])c(N(c2ccc3c(c2)C(c2ccccc2)(c2ccccc2)c2ccccc2-3)c2ccc3ccccc3c2)c([2H])c([2H])c1-c1cccc2c1oc1ccccc12 ; [2H]c1c([2H])c(N(c2ccc(-c3cccc4ccccc34)cc2)c2ccc3c(c2)C(c2ccccc2)(c2ccccc2)c2ccccc2-3)c([2H])c([2H])c1-c1cccc2c1oc1ccccc12 ; [2H]c1c([2H])c(N(c2ccc(-c3cccc4c3oc3ccccc34)cc2)c2ccc3c(c2)C(c2ccccc2)(c2ccccc2)c2ccccc2-3)c([2H])c([2H])c1-c1cccc2ccccc12 ; [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1cccc2c1oc1ccccc12]

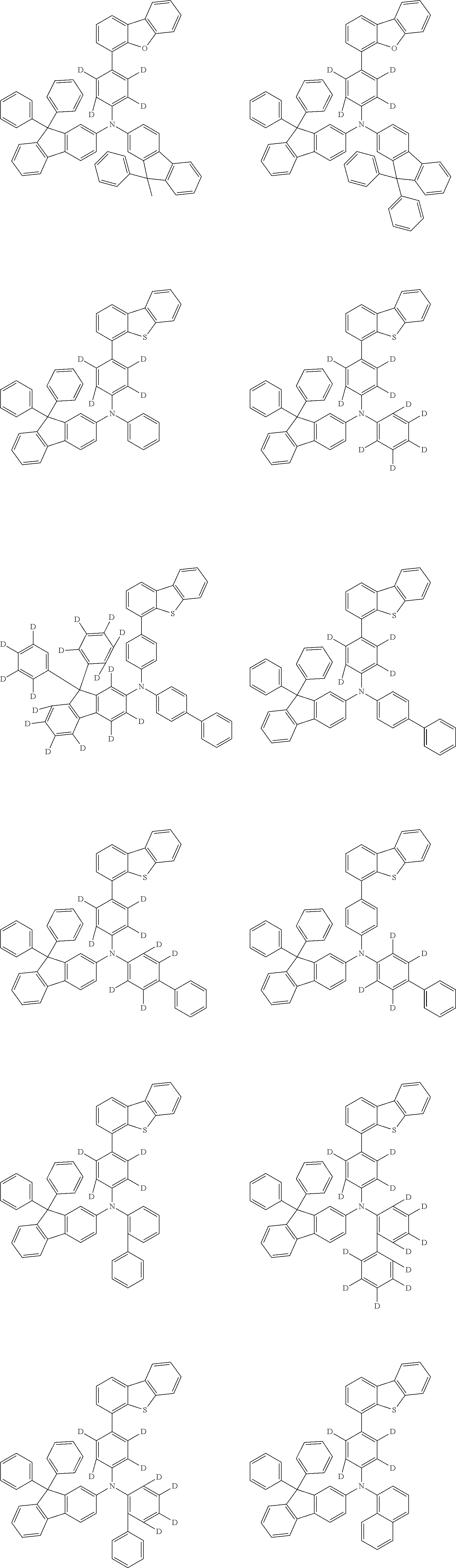

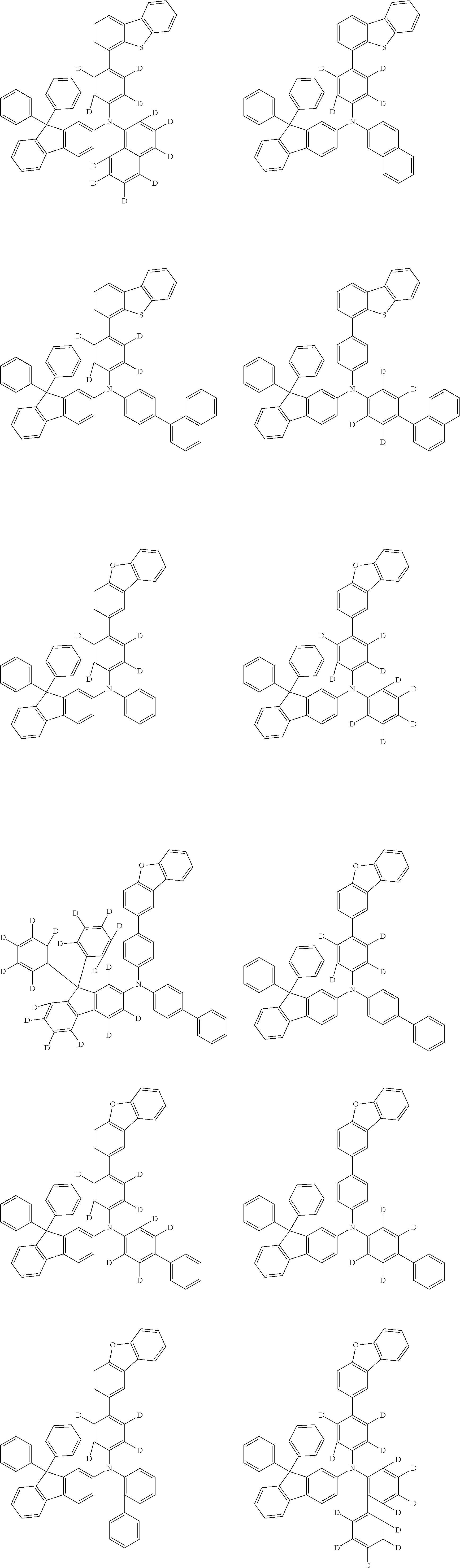

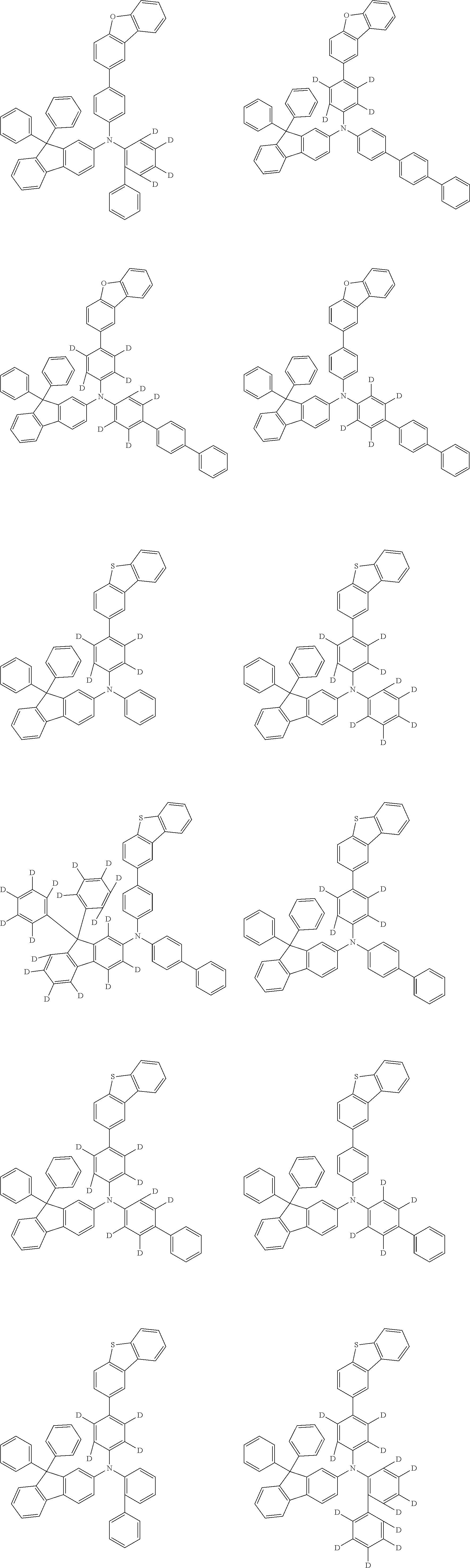

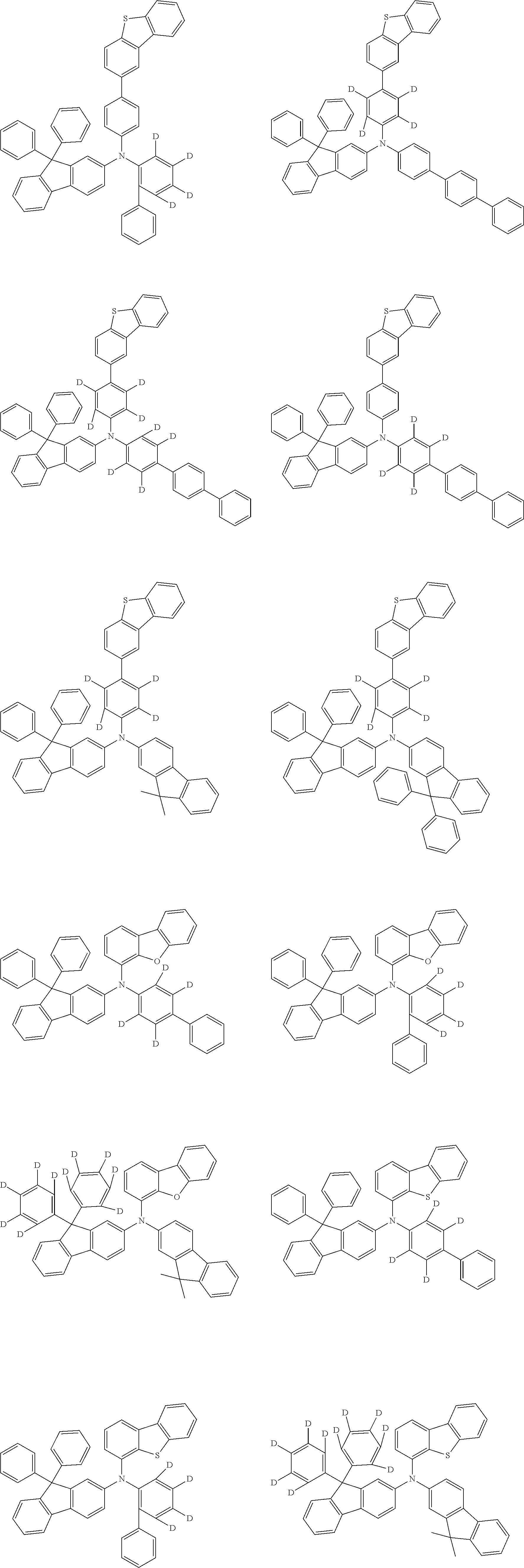

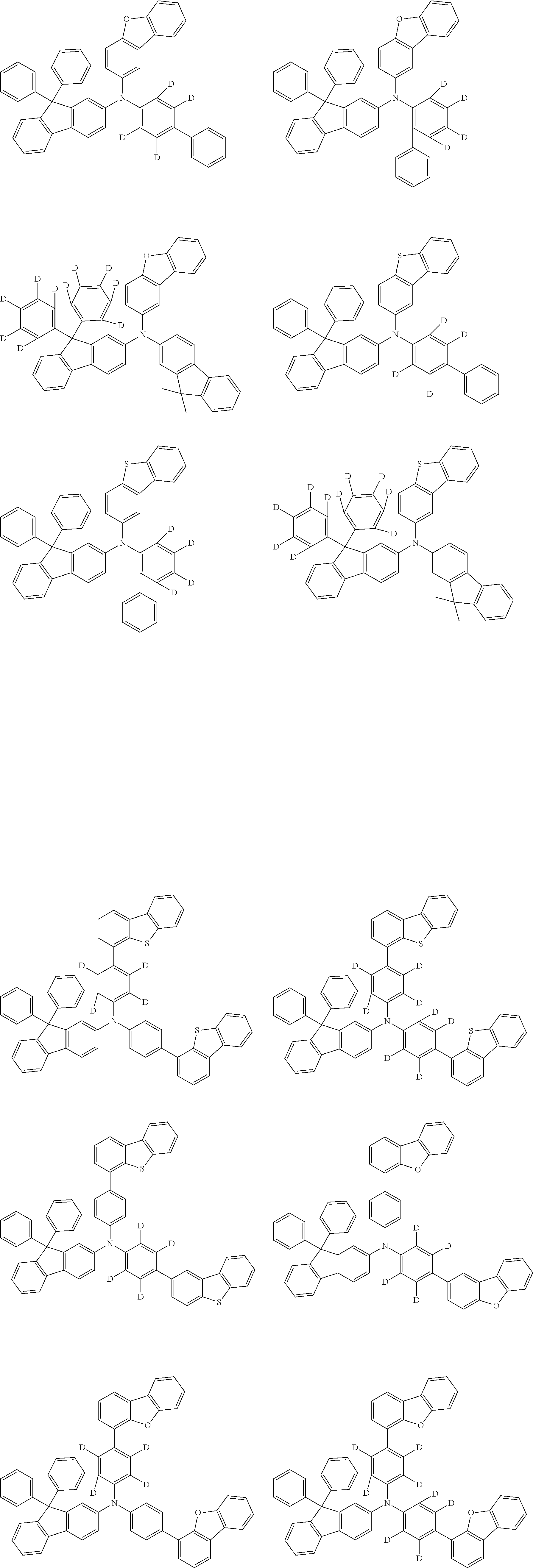

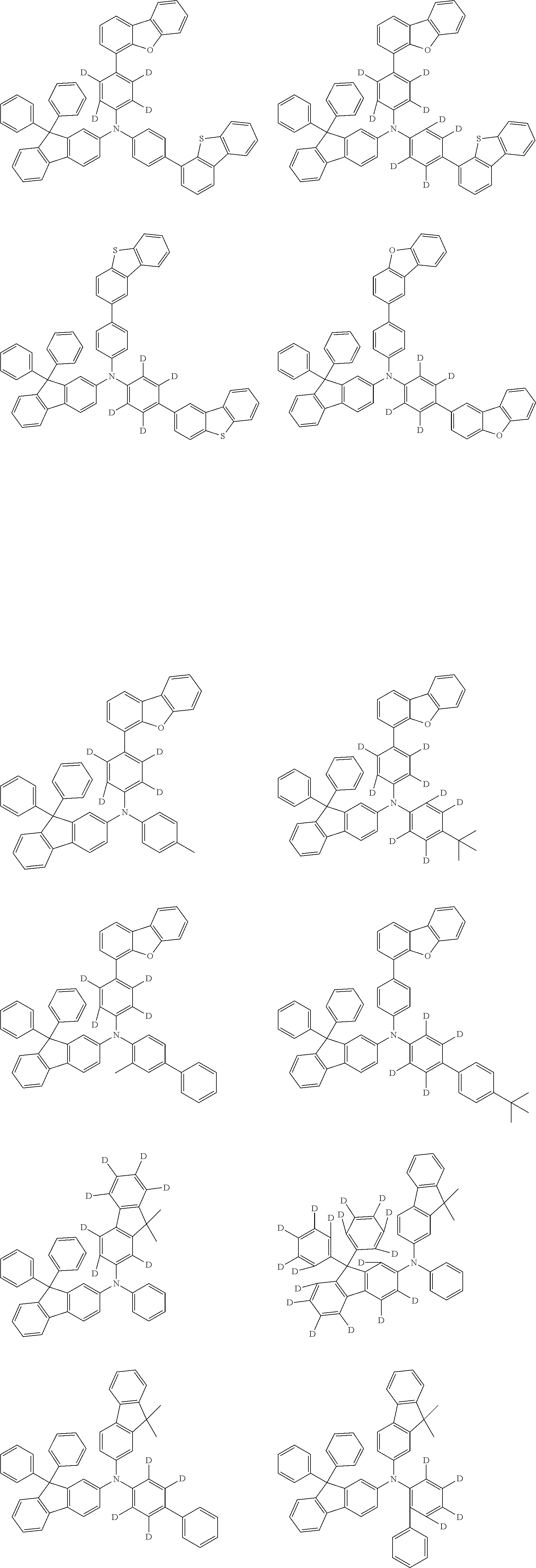

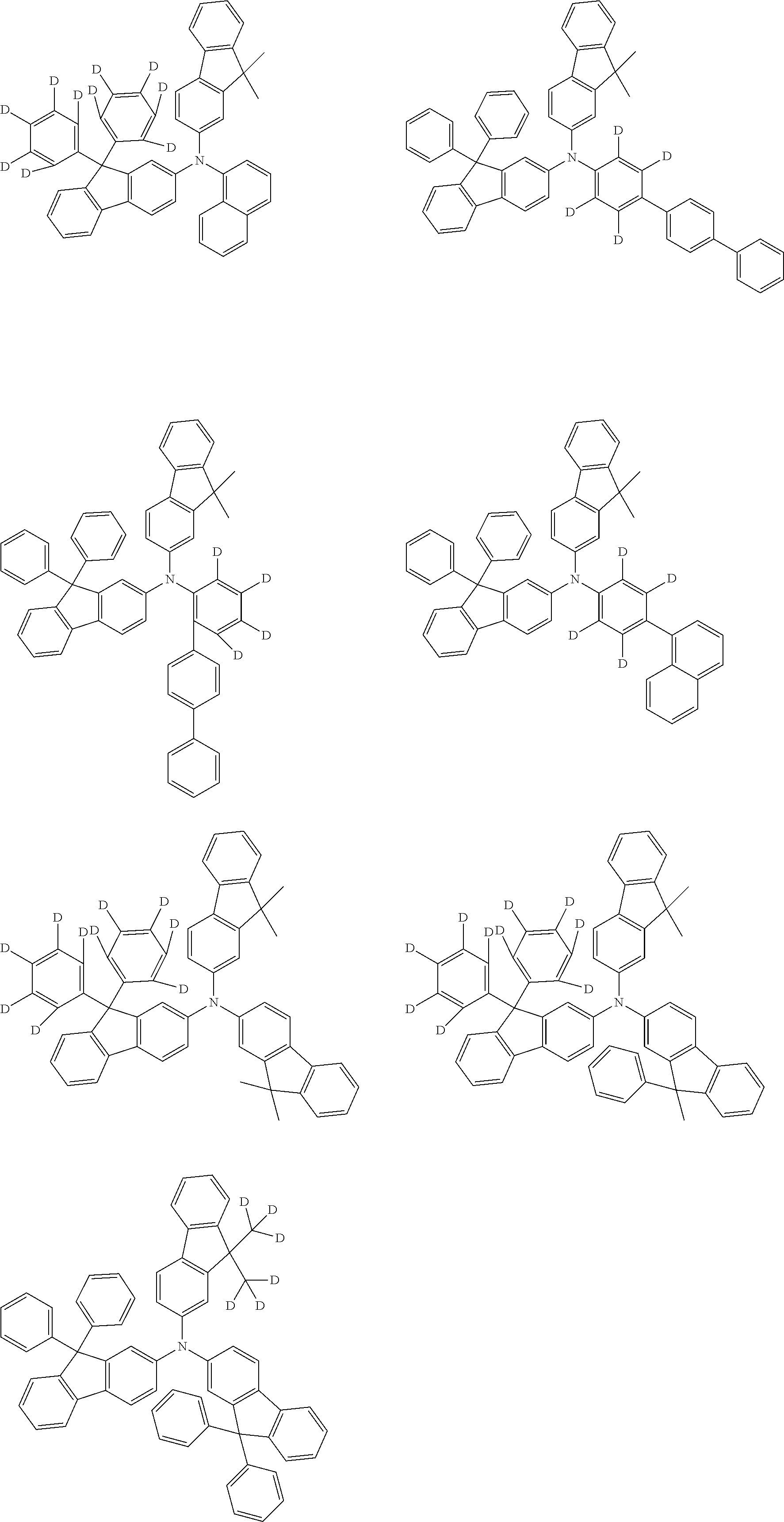

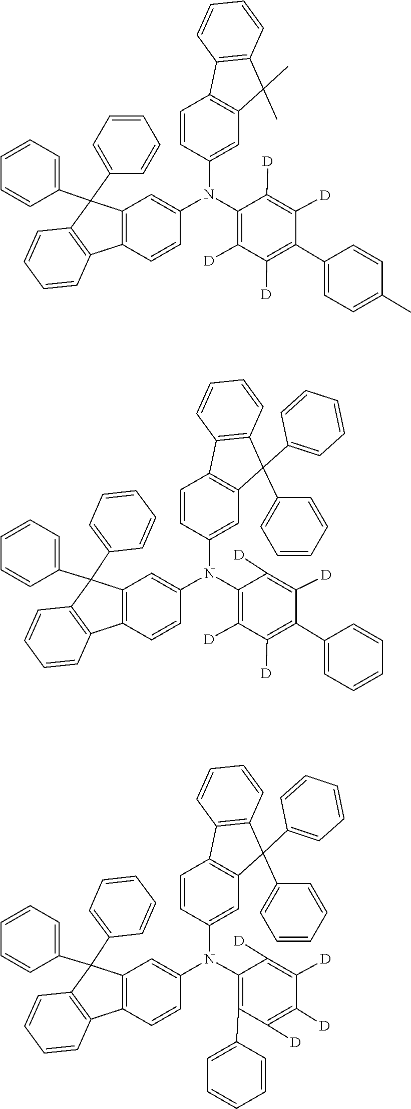 [SMILES: [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1ccc(C)cc1 ; [2H]c1c([2H])c(N(c2ccc3c(c2)C(c2ccccc2)(c2ccccc2)c2ccccc2-3)c2ccc3c(c2)C(c2ccccc2)(c2ccccc2)c2ccccc2-3)c([2H])c([2H])c1-c1ccccc1 ; [2H]c1c([2H])c([2H])c(N(c2ccc3c(c2)C(c2ccccc2)(c2ccccc2)c2ccccc2-3)c2ccc3c(c2)C(c2ccccc2)(c2ccccc2)c2ccccc2-3)c(-c2ccccc2)c1[2H]]

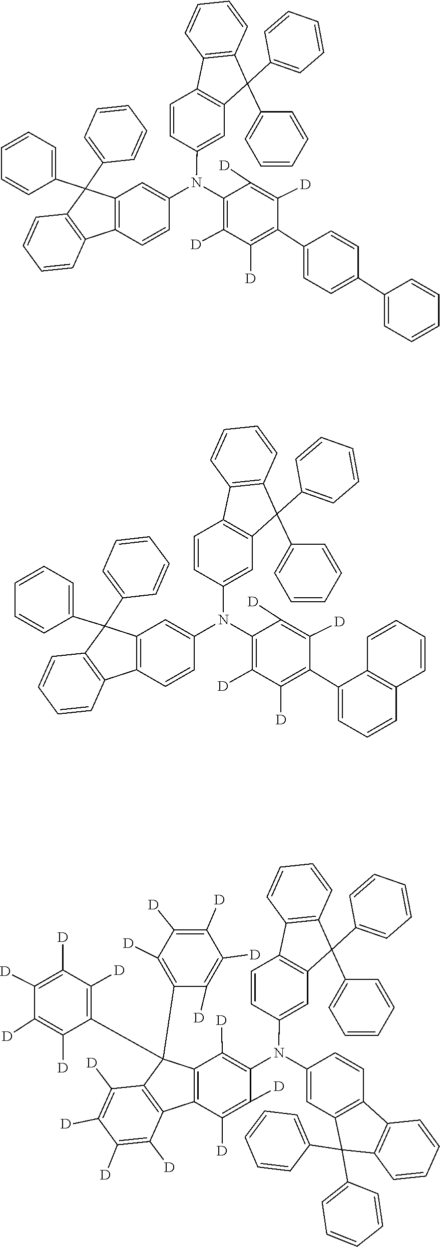

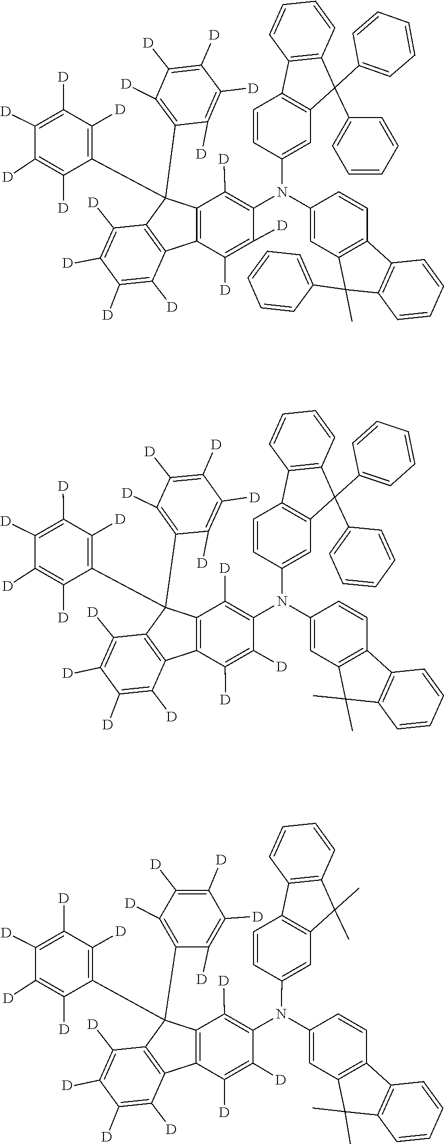

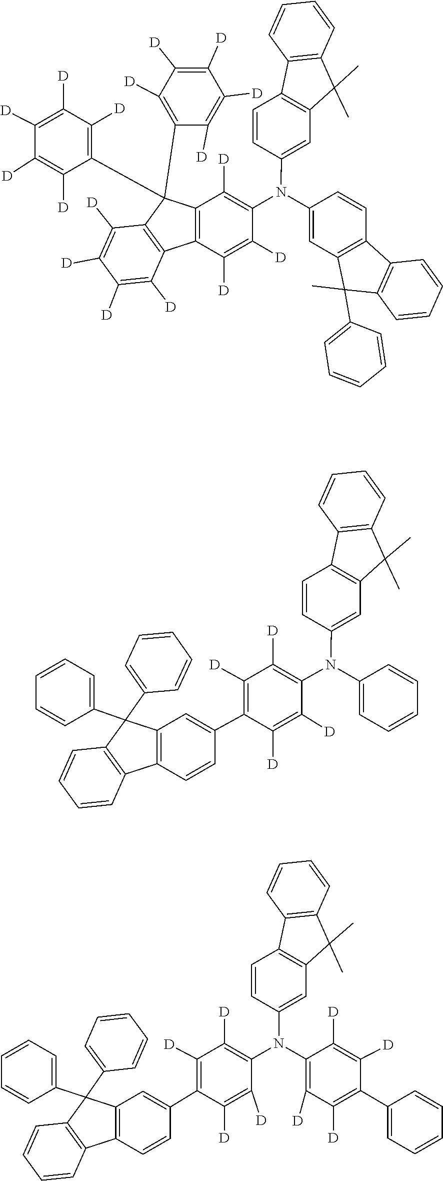 [SMILES: [2H]c1c([2H])c([2H])c(C2(c3c([2H])c([2H])c([2H])c([2H])c3[2H])c3c([2H])c([2H])c([2H])c([2H])c3-c3c([2H])c([2H])c(N(c4ccc5c(c4)C(C)(C)c4ccccc4-5)c4ccc5c(c4)C(C)(c4ccccc4)c4ccccc4-5)c([2H])c32)c([2H])c1[2H] ; [2H]c1c([2H])c(N(c2ccccc2)c2ccc3c(c2)C(C)(C)c2ccccc2-3)c([2H])c([2H])c1-c1ccc2c(c1)C(c1ccccc1)(c1ccccc1)c1ccccc1-2 ; [2H]c1c([2H])c(N(c2ccc3c(c2)C(C)(C)c2ccccc2-3)c2c([2H])c([2H])c(-c3ccc4c(c3)C(c3ccccc3)(c3ccccc3)c3ccccc3-4)c([2H])c2[2H])c([2H])c([2H])c1-c1ccccc1]

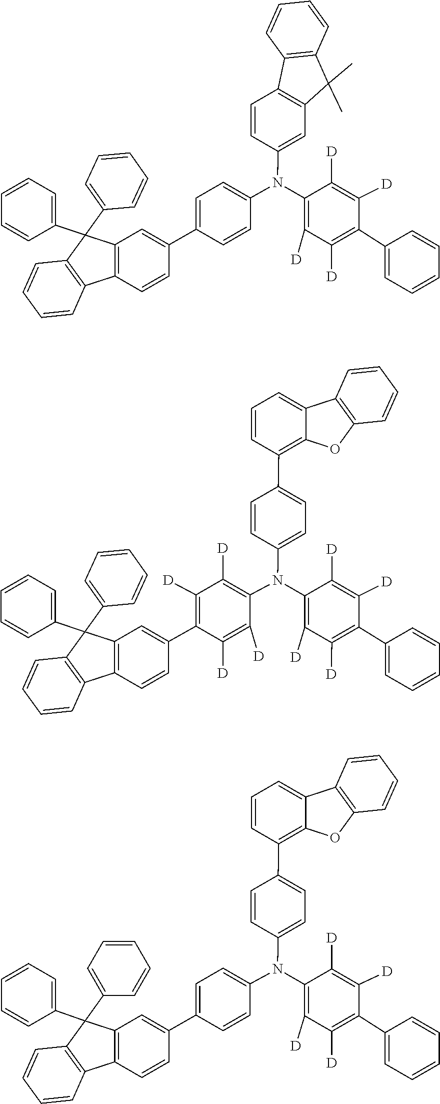 [SMILES: [2H]c1c([2H])c(N(c2ccc(-c3ccc4c(c3)C(c3ccccc3)(c3ccccc3)c3ccccc3-4)cc2)c2ccc3c(c2)C(C)(C)c2ccccc2-3)c([2H])c([2H])c1-c1ccccc1 ; [2H]c1c([2H])c(N(c2ccc(-c3cccc4c3oc3ccccc34)cc2)c2c([2H])c([2H])c(-c3ccc4c(c3)C(c3ccccc3)(c3ccccc3)c3ccccc3-4)c([2H])c2[2H])c([2H])c([2H])c1-c1ccccc1 ; [2H]c1c([2H])c(N(c2ccc(-c3ccc4c(c3)C(c3ccccc3)(c3ccccc3)c3ccccc3-4)cc2)c2ccc(-c3cccc4c3oc3ccccc34)cc2)c([2H])c([2H])c1-c1ccccc1]

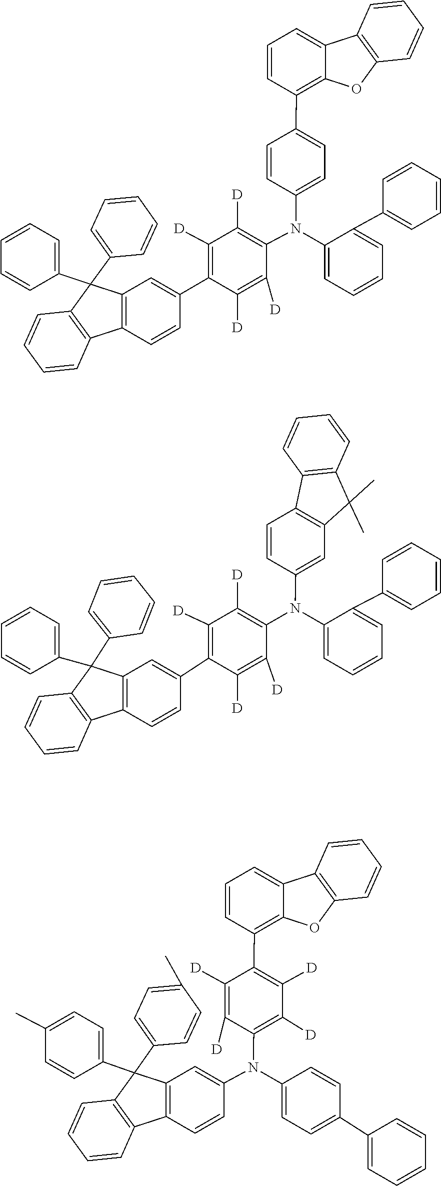 [SMILES: [2H]c1c([2H])c(N(c2ccc(-c3cccc4c3oc3ccccc34)cc2)c2ccccc2-c2ccccc2)c([2H])c([2H])c1-c1ccc2c(c1)C(c1ccccc1)(c1ccccc1)c1ccccc1-2 ; [2H]c1c([2H])c(N(c2ccc3c(c2)C(C)(C)c2ccccc2-3)c2ccccc2-c2ccccc2)c([2H])c([2H])c1-c1ccc2c(c1)C(c1ccccc1)(c1ccccc1)c1ccccc1-2 ; [2H]c1c([2H])c(N(c2ccc(-c3ccccc3)cc2)c2ccc3c(c2)C(c2ccc(C)cc2)(c2ccc(C)cc2)c2ccccc2-3)c([2H])c([2H])c1-c1cccc2c1oc1ccccc12]

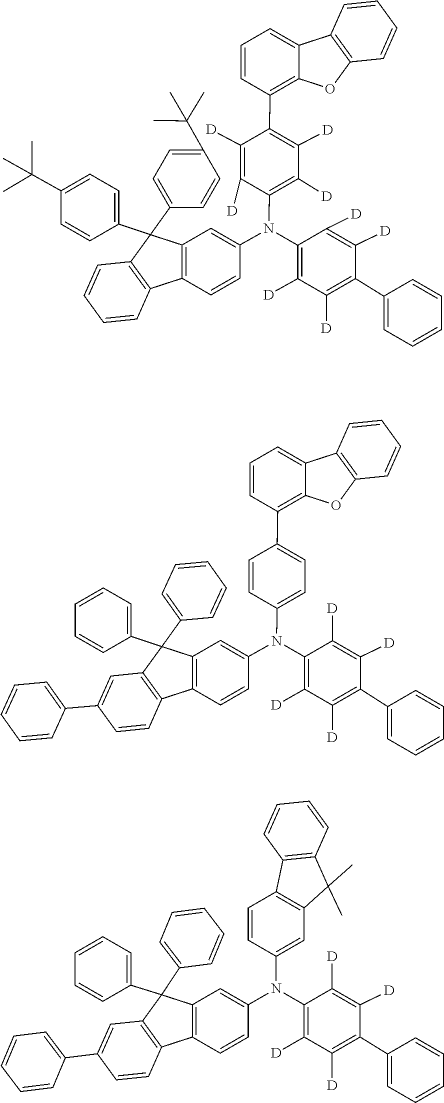 [SMILES: [2H]c1c([2H])c(N(c2ccc3c(c2)C(c2ccc(C(C)(C)C)cc2)(c2ccc(C(C)(C)C)cc2)c2ccccc2-3)c2c([2H])c([2H])c(-c3cccc4c3oc3ccccc34)c([2H])c2[2H])c([2H])c([2H])c1-c1ccccc1 ; [2H]c1c([2H])c(N(c2ccc(-c3cccc4c3oc3ccccc34)cc2)c2ccc3c(c2)C(c2ccccc2)(c2ccccc2)c2cc(-c4ccccc4)ccc2-3)c([2H])c([2H])c1-c1ccccc1 ; [2H]c1c([2H])c(N(c2ccc3c(c2)C(C)(C)c2ccccc2-3)c2ccc3c(c2)C(c2ccccc2)(c2ccccc2)c2cc(-c4ccccc4)ccc2-3)c([2H])c([2H])c1-c1ccccc1]

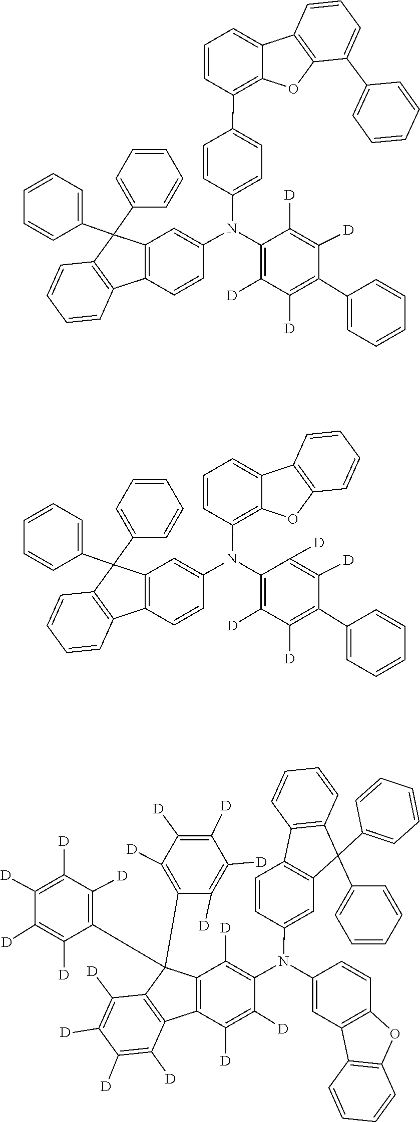 [SMILES: [2H]c1c([2H])c(N(c2ccc(-c3cccc4c3oc3c(-c5ccccc5)cccc34)cc2)c2ccc3c(c2)C(c2ccccc2)(c2ccccc2)c2ccccc2-3)c([2H])c([2H])c1-c1ccccc1 ; [2H]c1c([2H])c(N(c2ccc3c(c2)C(c2ccccc2)(c2ccccc2)c2ccccc2-3)c2cccc3c2oc2ccccc23)c([2H])c([2H])c1-c1ccccc1 ; [2H]c1c([2H])c([2H])c(C2(c3c([2H])c([2H])c([2H])c([2H])c3[2H])c3c([2H])c([2H])c([2H])c([2H])c3-c3c([2H])c([2H])c(N(c4ccc5c(c4)C(c4ccccc4)(c4ccccc4)c4ccccc4-5)c4ccc5oc6ccccc6c5c4)c([2H])c32)c([2H])c1[2H]]

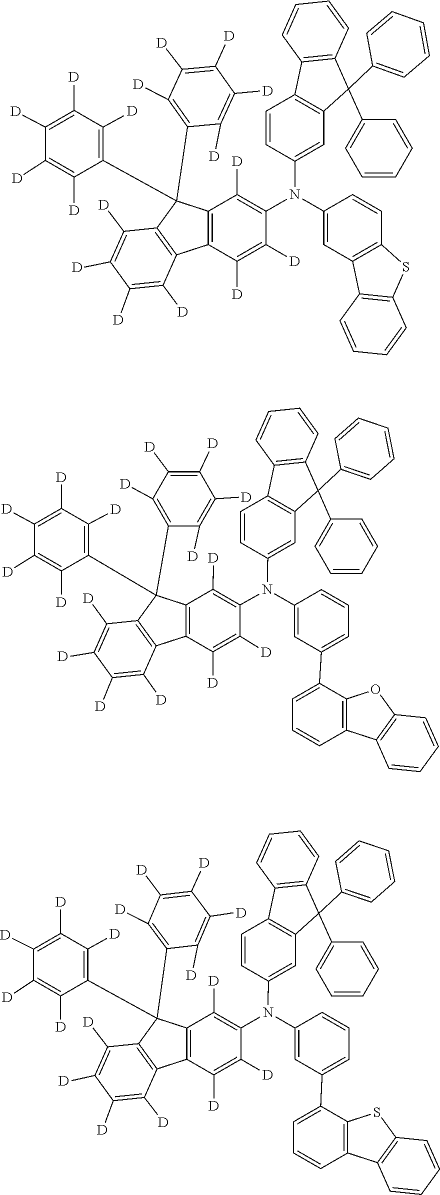

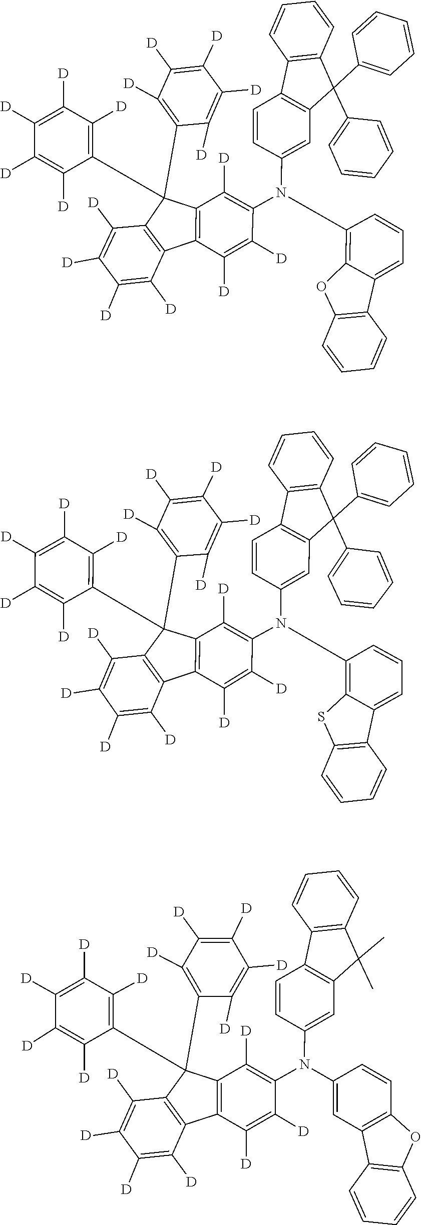

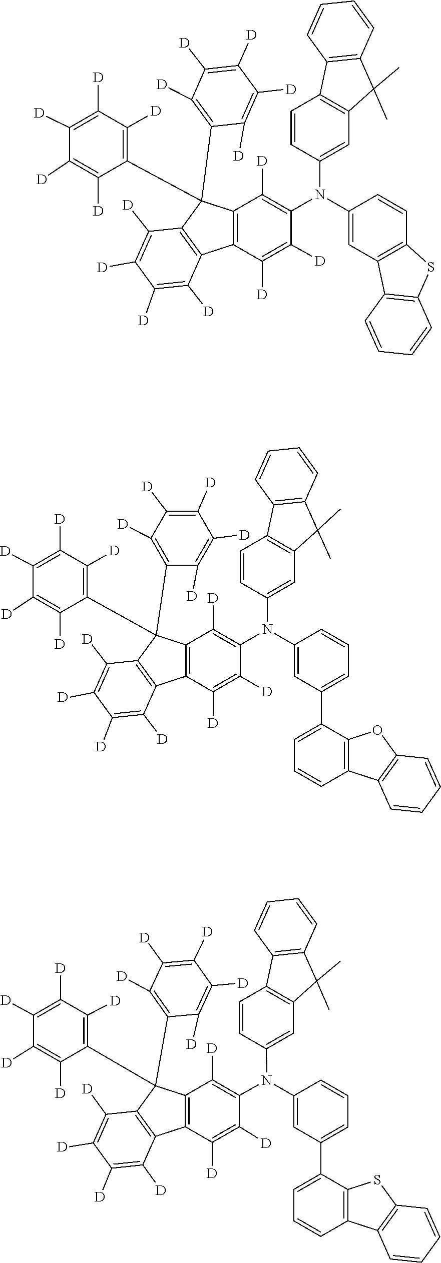

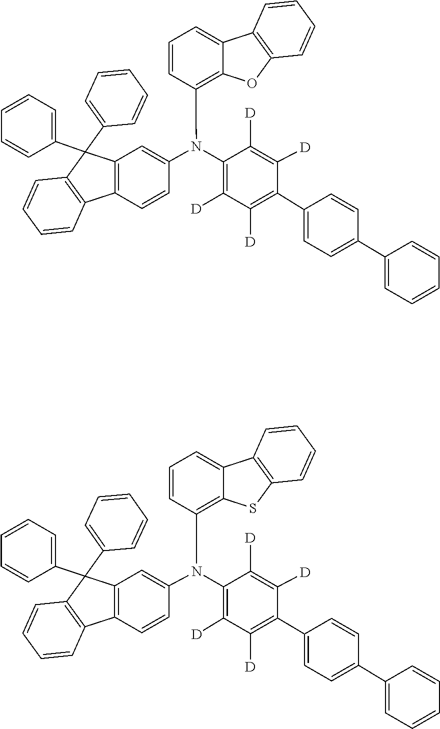 [SMILES: [2H]c1c([2H])c(N(c2ccc3c(c2)C(c2ccccc2)(c2ccccc2)c2ccccc2-3)c2cccc3c2oc2ccccc23)c([2H])c([2H])c1-c1ccc(-c2ccccc2)cc1 ; [2H]c1c([2H])c(N(c2ccc3c(c2)C(c2ccccc2)(c2ccccc2)c2ccccc2-3)c2cccc3c2sc2ccccc23)c([2H])c([2H])c1-c1ccc(-c2ccccc2)cc1]

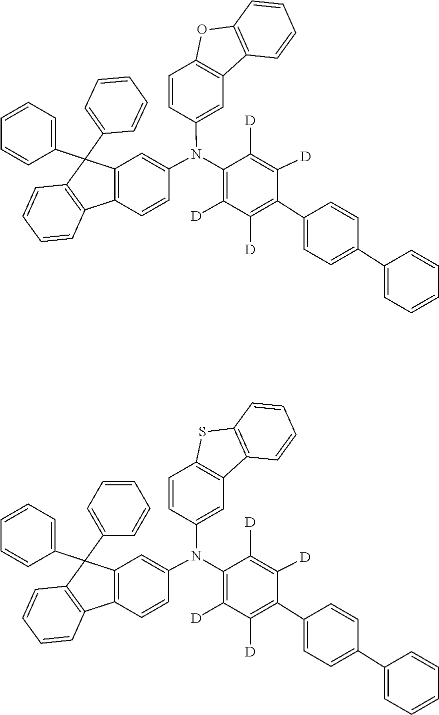 [SMILES: [2H]c1c([2H])c(N(c2ccc3c(c2)C(c2ccccc2)(c2ccccc2)c2ccccc2-3)c2ccc3oc4ccccc4c3c2)c([2H])c([2H])c1-c1ccc(-c2ccccc2)cc1 ; [2H]c1c([2H])c(N(c2ccc3c(c2)C(c2ccccc2)(c2ccccc2)c2ccccc2-3)c2ccc3sc4ccccc4c3c2)c([2H])c([2H])c1-c1ccc(-c2ccccc2)cc1]

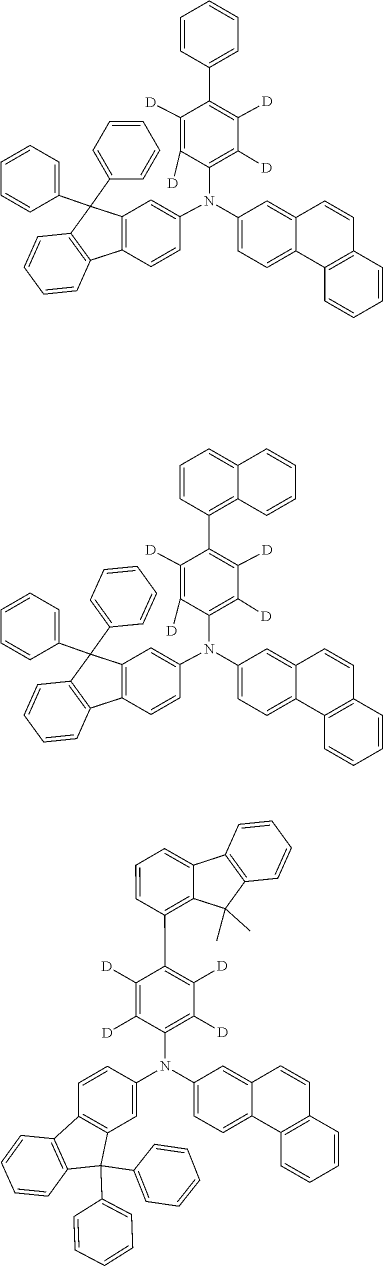 [SMILES: [2H]c1c([2H])c(N(c2ccc3c(c2)C(c2ccccc2)(c2ccccc2)c2ccccc2-3)c2ccc3c(ccc4ccccc43)c2)c([2H])c([2H])c1-c1ccccc1 ; [2H]c1c([2H])c(N(c2ccc3c(c2)C(c2ccccc2)(c2ccccc2)c2ccccc2-3)c2ccc3c(ccc4ccccc43)c2)c([2H])c([2H])c1-c1cccc2ccccc12 ; [2H]c1c([2H])c(N(c2ccc3c(c2)C(c2ccccc2)(c2ccccc2)c2ccccc2-3)c2ccc3c(ccc4ccccc43)c2)c([2H])c([2H])c1-c1cccc2c1C(C)(C)c1ccccc1-2]

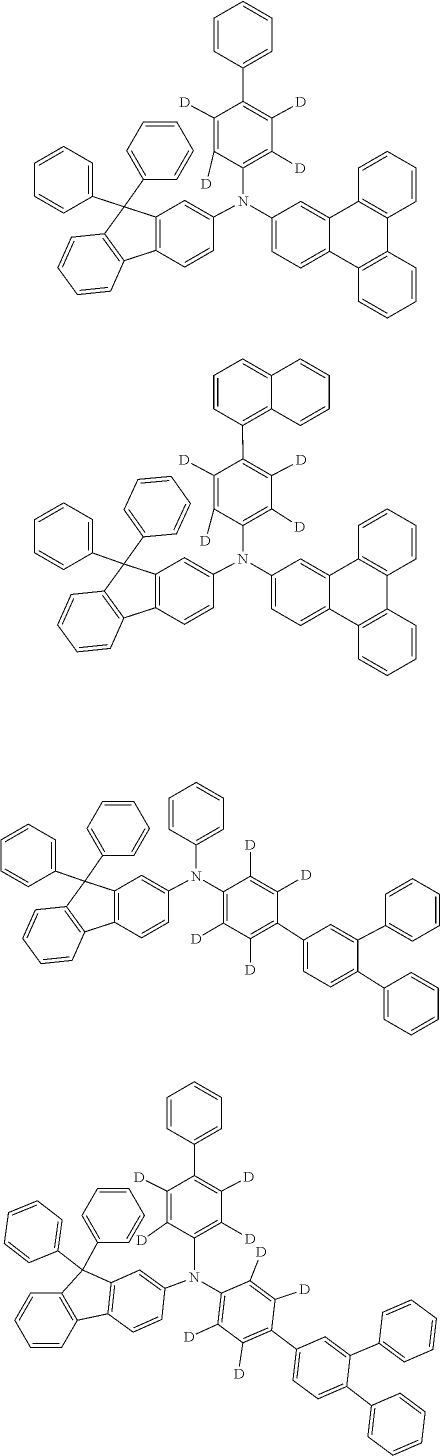

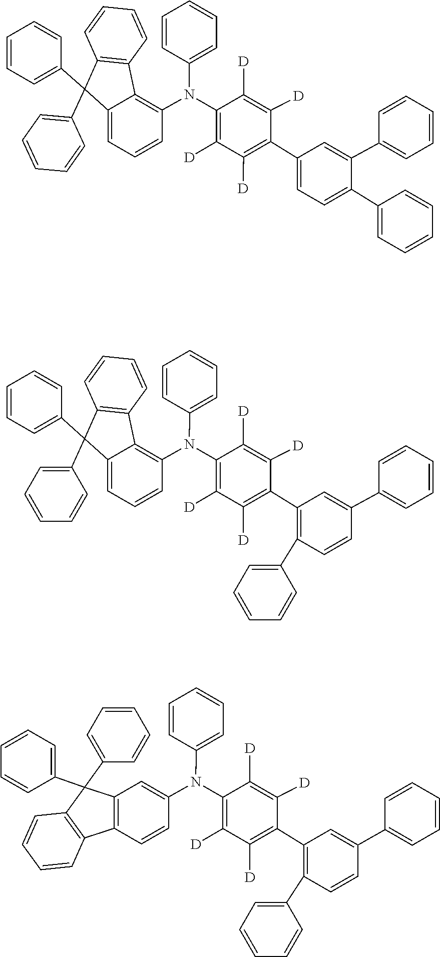 [SMILES: [2H]c1c([2H])c(N(c2ccccc2)c2cccc3c2-c2ccccc2C3(c2ccccc2)c2ccccc2)c([2H])c([2H])c1-c1ccc(-c2ccccc2)c(-c2ccccc2)c1 ; [2H]c1c([2H])c(N(c2ccccc2)c2cccc3c2-c2ccccc2C3(c2ccccc2)c2ccccc2)c([2H])c([2H])c1-c1cc(-c2ccccc2)ccc1-c1ccccc1 ; [2H]c1c([2H])c(N(c2ccccc2)c2ccc3c(c2)C(c2ccccc2)(c2ccccc2)c2ccccc2-3)c([2H])c([2H])c1-c1cc(-c2ccccc2)ccc1-c1ccccc1]

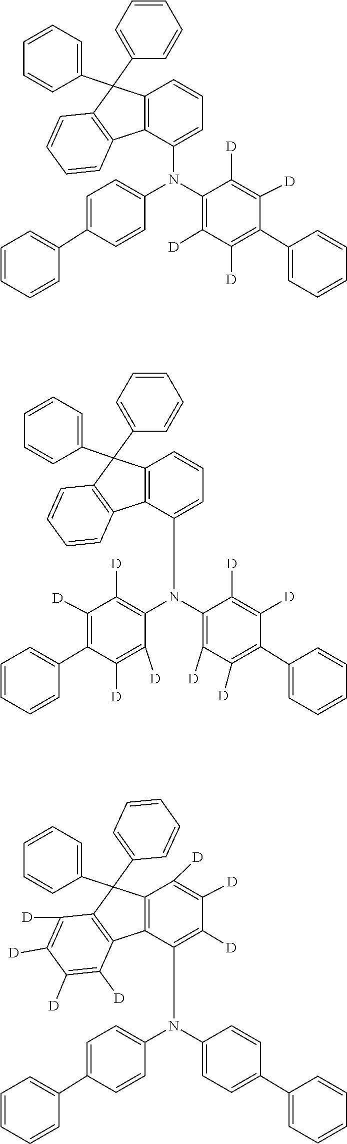 [SMILES: [2H]c1c([2H])c(N(c2ccc(-c3ccccc3)cc2)c2cccc3c2-c2ccccc2C3(c2ccccc2)c2ccccc2)c([2H])c([2H])c1-c1ccccc1 ; [2H]c1c([2H])c(N(c2cccc3c2-c2ccccc2C3(c2ccccc2)c2ccccc2)c2c([2H])c([2H])c(-c3ccccc3)c([2H])c2[2H])c([2H])c([2H])c1-c1ccccc1 ; [2H]c1c([2H])c([2H])c2c(c1[2H])-c1c(N(c3ccc(-c4ccccc4)cc3)c3ccc(-c4ccccc4)cc3)c([2H])c([2H])c([2H])c1C2(c1ccccc1)c1ccccc1]

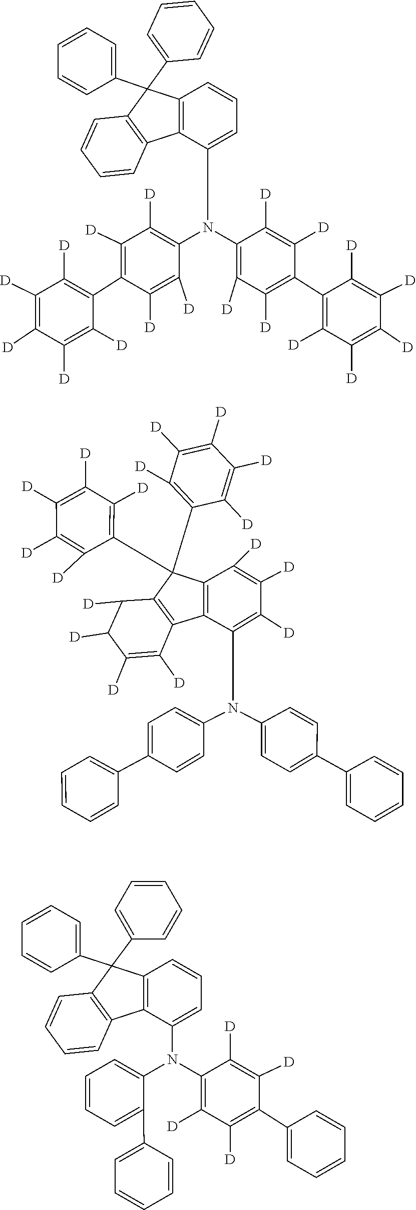 [SMILES: [2H]c1c([2H])c([2H])c(-c2c([2H])c([2H])c(N(c3cccc4c3-c3ccccc3C4(c3ccccc3)c3ccccc3)c3c([2H])c([2H])c(-c4c([2H])c([2H])c([2H])c([2H])c4[2H])c([2H])c3[2H])c([2H])c2[2H])c([2H])c1[2H] ; [2H]C1=C([2H])C([2H])C([2H])C2=C1c1c(N(c3ccc(-c4ccccc4)cc3)c3ccc(-c4ccccc4)cc3)c([2H])c([2H])c([2H])c1C2(c1c([2H])c([2H])c([2H])c([2H])c1[2H])c1c([2H])c([2H])c([2H])c([2H])c1[2H] ; [2H]c1c([2H])c(N(c2ccccc2-c2ccccc2)c2cccc3c2-c2ccccc2C3(c2ccccc2)c2ccccc2)c([2H])c([2H])c1-c1ccccc1]

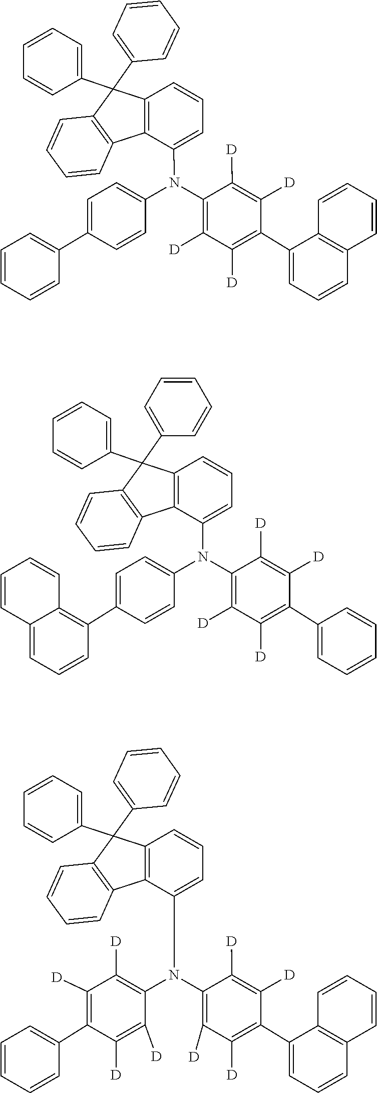 [SMILES: [2H]c1c([2H])c(N(c2ccc(-c3ccccc3)cc2)c2cccc3c2-c2ccccc2C3(c2ccccc2)c2ccccc2)c([2H])c([2H])c1-c1cccc2ccccc12 ; [2H]c1c([2H])c(N(c2ccc(-c3cccc4ccccc34)cc2)c2cccc3c2-c2ccccc2C3(c2ccccc2)c2ccccc2)c([2H])c([2H])c1-c1ccccc1 ; [2H]c1c([2H])c(N(c2cccc3c2-c2ccccc2C3(c2ccccc2)c2ccccc2)c2c([2H])c([2H])c(-c3cccc4ccccc34)c([2H])c2[2H])c([2H])c([2H])c1-c1ccccc1]

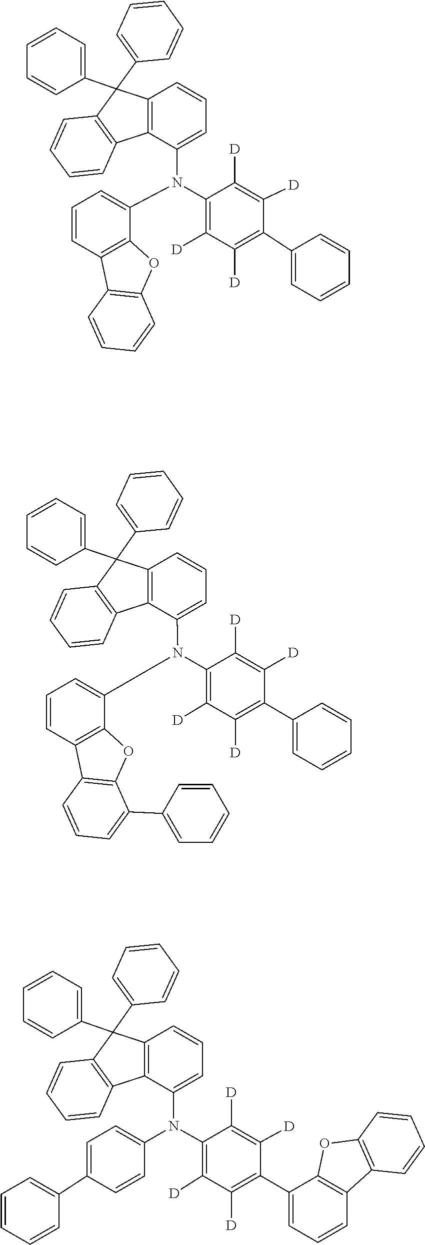 [SMILES: [2H]c1c([2H])c(N(c2cccc3c2-c2ccccc2C3(c2ccccc2)c2ccccc2)c2cccc3c2oc2ccccc23)c([2H])c([2H])c1-c1ccccc1 ; [2H]c1c([2H])c(N(c2cccc3c2-c2ccccc2C3(c2ccccc2)c2ccccc2)c2cccc3c2oc2c(-c4ccccc4)cccc23)c([2H])c([2H])c1-c1ccccc1 ; [2H]c1c([2H])c(N(c2ccc(-c3ccccc3)cc2)c2cccc3c2-c2ccccc2C3(c2ccccc2)c2ccccc2)c([2H])c([2H])c1-c1cccc2c1oc1ccccc12]

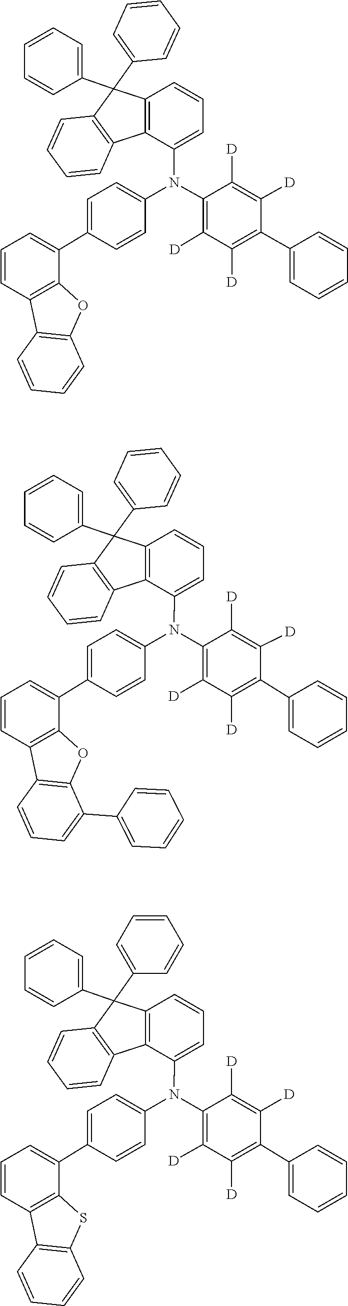 [SMILES: [2H]c1c([2H])c(N(c2ccc(-c3cccc4c3oc3ccccc34)cc2)c2cccc3c2-c2ccccc2C3(c2ccccc2)c2ccccc2)c([2H])c([2H])c1-c1ccccc1 ; [2H]c1c([2H])c(N(c2ccc(-c3cccc4c3oc3c(-c5ccccc5)cccc34)cc2)c2cccc3c2-c2ccccc2C3(c2ccccc2)c2ccccc2)c([2H])c([2H])c1-c1ccccc1 ; [2H]c1c([2H])c(N(c2ccc(-c3cccc4c3sc3ccccc34)cc2)c2cccc3c2-c2ccccc2C3(c2ccccc2)c2ccccc2)c([2H])c([2H])c1-c1ccccc1]

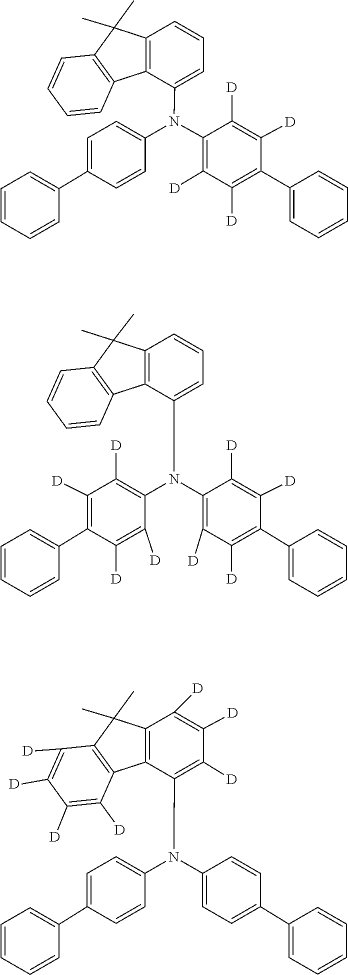 [SMILES: [2H]c1c([2H])c(N(c2ccc(-c3ccccc3)cc2)c2cccc3c2-c2ccccc2C3(C)C)c([2H])c([2H])c1-c1ccccc1 ; [2H]c1c([2H])c(N(c2cccc3c2-c2ccccc2C3(C)C)c2c([2H])c([2H])c(-c3ccccc3)c([2H])c2[2H])c([2H])c([2H])c1-c1ccccc1 ; [2H]c1c([2H])c([2H])c2c(c1[2H])-c1c(N(c3ccc(-c4ccccc4)cc3)c3ccc(-c4ccccc4)cc3)c([2H])c([2H])c([2H])c1C2(C)C]

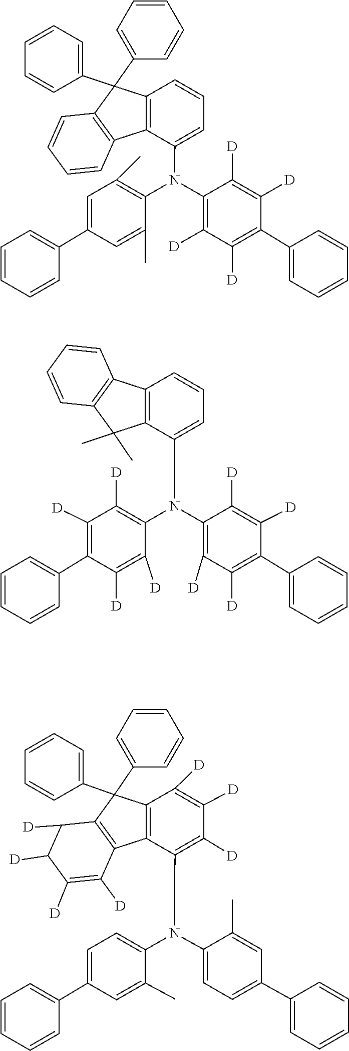 [SMILES: [2H]c1c([2H])c(N(c2cccc3c2-c2ccccc2C3(c2ccccc2)c2ccccc2)c2c(C)cc(-c3ccccc3)cc2C)c([2H])c([2H])c1-c1ccccc1 ; [2H]c1c([2H])c(N(c2cccc3c2C(C)(C)c2ccccc2-3)c2c([2H])c([2H])c(-c3ccccc3)c([2H])c2[2H])c([2H])c([2H])c1-c1ccccc1 ; [2H]C1=C([2H])C([2H])C([2H])C2=C1c1c(N(c3ccc(-c4ccccc4)cc3C)c3ccc(-c4ccccc4)cc3C)c([2H])c([2H])c([2H])c1C2(c1ccccc1)c1ccccc1]

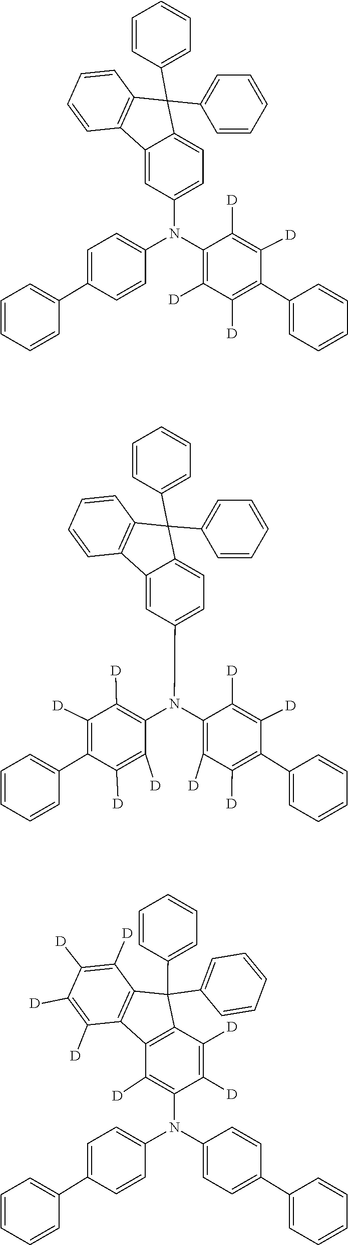 [SMILES: [2H]c1c([2H])c(N(c2ccc(-c3ccccc3)cc2)c2ccc3c(c2)-c2ccccc2C3(c2ccccc2)c2ccccc2)c([2H])c([2H])c1-c1ccccc1 ; [2H]c1c([2H])c(N(c2ccc3c(c2)-c2ccccc2C3(c2ccccc2)c2ccccc2)c2c([2H])c([2H])c(-c3ccccc3)c([2H])c2[2H])c([2H])c([2H])c1-c1ccccc1 ; [2H]c1c([2H])c([2H])c2c(c1[2H])-c1c([2H])c(N(c3ccc(-c4ccccc4)cc3)c3ccc(-c4ccccc4)cc3)c([2H])c([2H])c1C2(c1ccccc1)c1ccccc1]

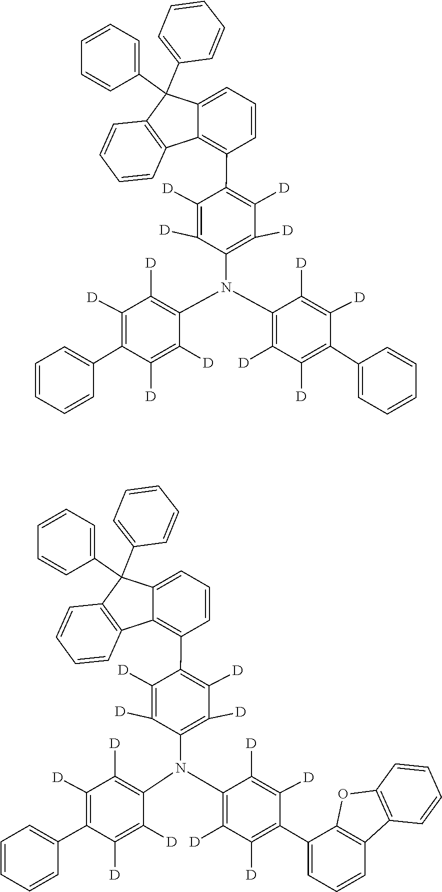 [SMILES: [2H]c1c([2H])c(N(c2c([2H])c([2H])c(-c3ccccc3)c([2H])c2[2H])c2c([2H])c([2H])c(-c3cccc4c3-c3ccccc3C4(c3ccccc3)c3ccccc3)c([2H])c2[2H])c([2H])c([2H])c1-c1ccccc1 ; [2H]c1c([2H])c(N(c2c([2H])c([2H])c(-c3cccc4c3-c3ccccc3C4(c3ccccc3)c3ccccc3)c([2H])c2[2H])c2c([2H])c([2H])c(-c3cccc4c3oc3ccccc34)c([2H])c2[2H])c([2H])c([2H])c1-c1ccccc1]

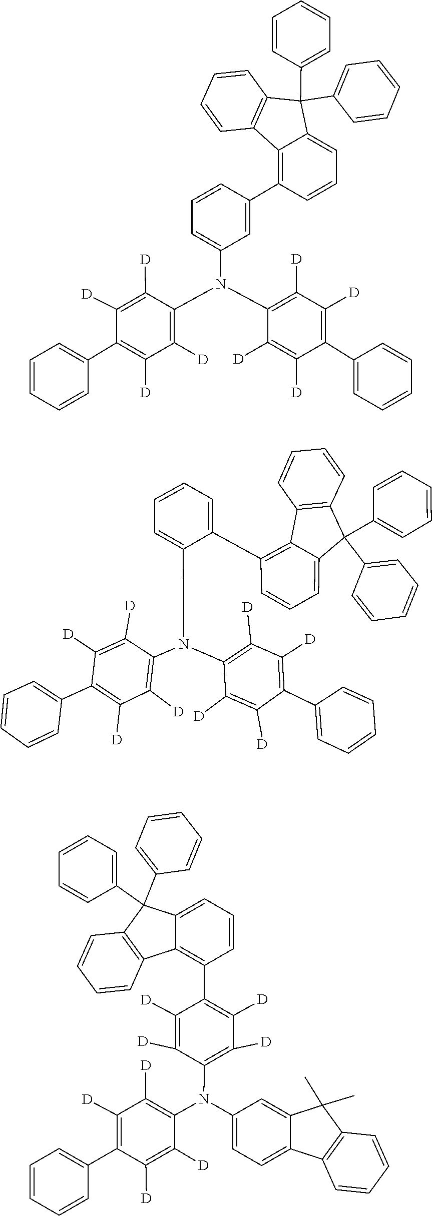 [SMILES: [2H]c1c([2H])c(N(c2cccc(-c3cccc4c3-c3ccccc3C4(c3ccccc3)c3ccccc3)c2)c2c([2H])c([2H])c(-c3ccccc3)c([2H])c2[2H])c([2H])c([2H])c1-c1ccccc1 ; [2H]c1c([2H])c(N(c2ccccc2-c2cccc3c2-c2ccccc2C3(c2ccccc2)c2ccccc2)c2c([2H])c([2H])c(-c3ccccc3)c([2H])c2[2H])c([2H])c([2H])c1-c1ccccc1 ; [2H]c1c([2H])c(N(c2ccc3c(c2)C(C)(C)c2ccccc2-3)c2c([2H])c([2H])c(-c3cccc4c3-c3ccccc3C4(c3ccccc3)c3ccccc3)c([2H])c2[2H])c([2H])c([2H])c1-c1ccccc1]

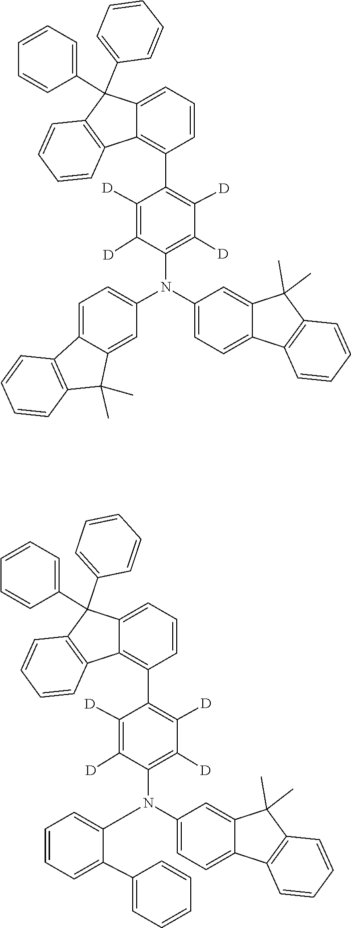 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1cccc2c1-c1ccccc1C2(c1ccccc1)c1ccccc1 ; [2H]c1c([2H])c(N(c2ccc3c(c2)C(C)(C)c2ccccc2-3)c2ccccc2-c2ccccc2)c([2H])c([2H])c1-c1cccc2c1-c1ccccc1C2(c1ccccc1)c1ccccc1]

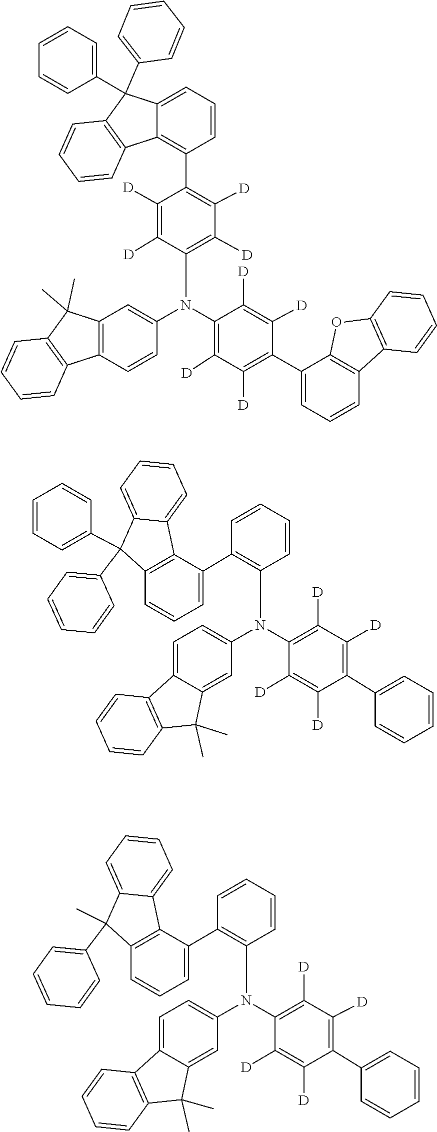 [SMILES: [2H]c1c([2H])c(N(c2ccc3c(c2)C(C)(C)c2ccccc2-3)c2c([2H])c([2H])c(-c3cccc4c3oc3ccccc34)c([2H])c2[2H])c([2H])c([2H])c1-c1cccc2c1-c1ccccc1C2(c1ccccc1)c1ccccc1 ; [2H]c1c([2H])c(N(c2ccc3c(c2)C(C)(C)c2ccccc2-3)c2ccccc2-c2cccc3c2-c2ccccc2C3(c2ccccc2)c2ccccc2)c([2H])c([2H])c1-c1ccccc1 ; [2H]c1c([2H])c(N(c2ccc3c(c2)C(C)(C)c2ccccc2-3)c2ccccc2-c2cccc3c2-c2ccccc2C3(C)c2ccccc2)c([2H])c([2H])c1-c1ccccc1]

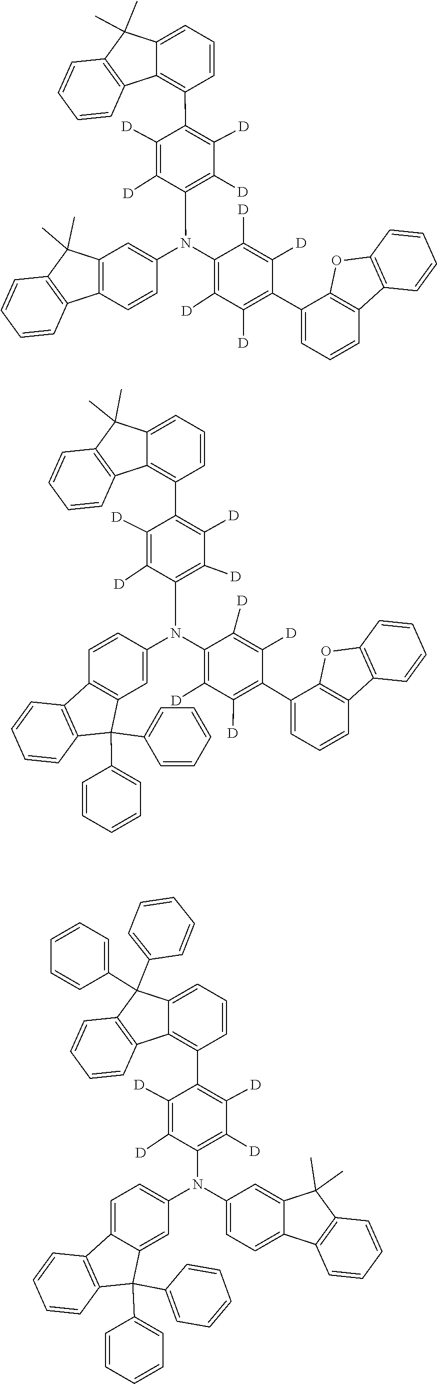 [SMILES: [2H]c1c([2H])c(N(c2ccc3c(c2)C(C)(C)c2ccccc2-3)c2c([2H])c([2H])c(-c3cccc4c3oc3ccccc34)c([2H])c2[2H])c([2H])c([2H])c1-c1cccc2c1-c1ccccc1C2(C)C ; [2H]c1c([2H])c(N(c2ccc3c(c2)C(c2ccccc2)(c2ccccc2)c2ccccc2-3)c2c([2H])c([2H])c(-c3cccc4c3oc3ccccc34)c([2H])c2[2H])c([2H])c([2H])c1-c1cccc2c1-c1ccccc1C2(C)C ; [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1cccc2c1-c1ccccc1C2(c1ccccc1)c1ccccc1]

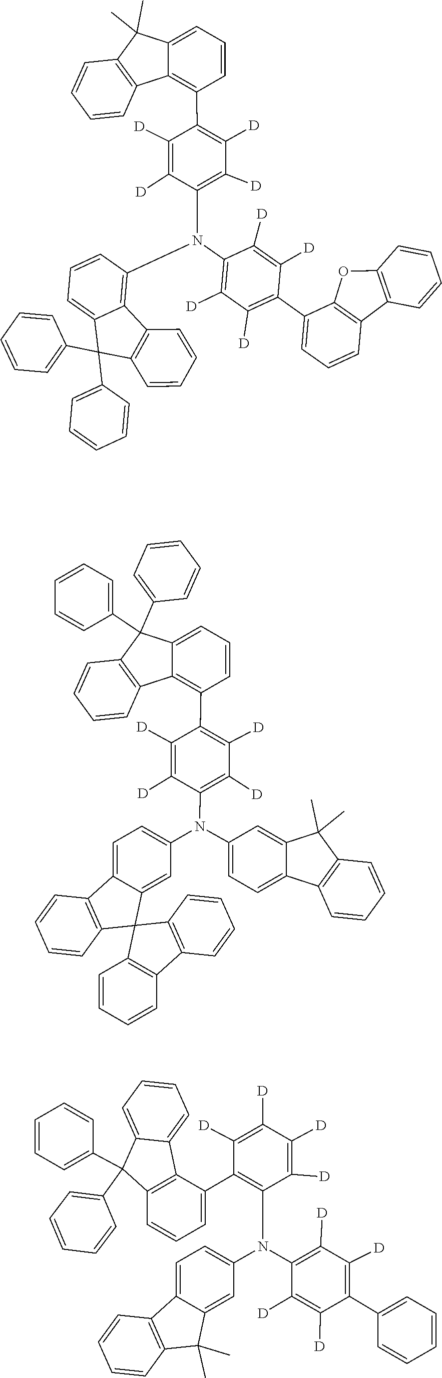 [SMILES: [2H]c1c([2H])c(N(c2cccc3c2-c2ccccc2C3(c2ccccc2)c2ccccc2)c2c([2H])c([2H])c(-c3cccc4c3oc3ccccc34)c([2H])c2[2H])c([2H])c([2H])c1-c1cccc2c1-c1ccccc1C2(C)C ; [2H]c1c([2H])c(N(c2ccc3c(c2)C(C)(C)c2ccccc2-3)c2ccc3c(c2)C2(c4ccccc4-c4ccccc42)c2ccccc2-3)c([2H])c([2H])c1-c1cccc2c1-c1ccccc1C2(c1ccccc1)c1ccccc1 ; [2H]c1c([2H])c([2H])c(N(c2ccc3c(c2)C(C)(C)c2ccccc2-3)c2c([2H])c([2H])c(-c3ccccc3)c([2H])c2[2H])c(-c2cccc3c2-c2ccccc2C3(c2ccccc2)c2ccccc2)c1[2H]]

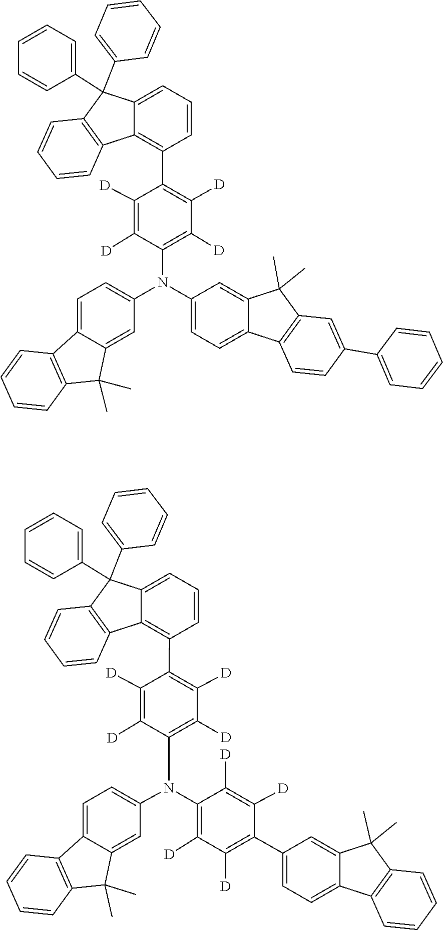 [SMILES: [2H]c1c([2H])c(N(c2ccc3c(c2)C(C)(C)c2ccccc2-3)c2ccc3c(c2)C(C)(C)c2cc(-c4ccccc4)ccc2-3)c([2H])c([2H])c1-c1cccc2c1-c1ccccc1C2(c1ccccc1)c1ccccc1 ; [2H]c1c([2H])c(N(c2ccc3c(c2)C(C)(C)c2ccccc2-3)c2c([2H])c([2H])c(-c3cccc4c3-c3ccccc3C4(c3ccccc3)c3ccccc3)c([2H])c2[2H])c([2H])c([2H])c1-c1ccc2c(c1)C(C)(C)c1ccccc1-2]

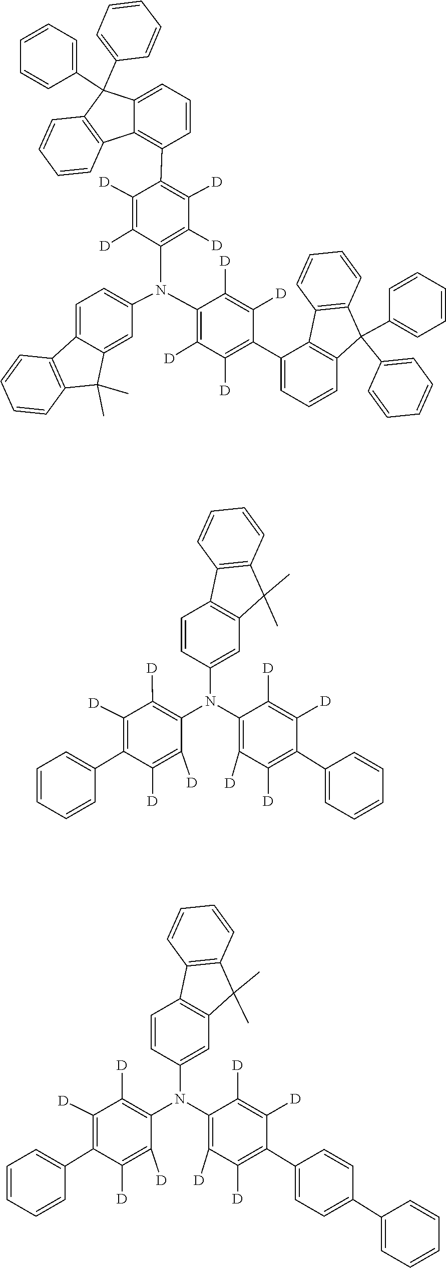 [SMILES: [2H]c1c([2H])c(N(c2ccc3c(c2)C(C)(C)c2ccccc2-3)c2c([2H])c([2H])c(-c3cccc4c3-c3ccccc3C4(c3ccccc3)c3ccccc3)c([2H])c2[2H])c([2H])c([2H])c1-c1cccc2c1-c1ccccc1C2(c1ccccc1)c1ccccc1 ; [2H]c1c([2H])c(N(c2ccc3c(c2)C(C)(C)c2ccccc2-3)c2c([2H])c([2H])c(-c3ccccc3)c([2H])c2[2H])c([2H])c([2H])c1-c1ccccc1 ; [2H]c1c([2H])c(N(c2ccc3c(c2)C(C)(C)c2ccccc2-3)c2c([2H])c([2H])c(-c3ccc(-c4ccccc4)cc3)c([2H])c2[2H])c([2H])c([2H])c1-c1ccccc1]

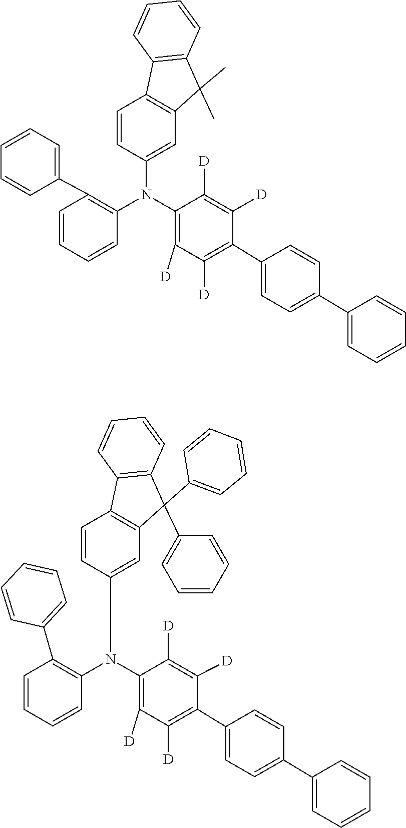 [SMILES: [2H]c1c([2H])c(N(c2ccc3c(c2)C(C)(C)c2ccccc2-3)c2ccccc2-c2ccccc2)c([2H])c([2H])c1-c1ccc(-c2ccccc2)cc1 ; [2H]c1c([2H])c(N(c2ccc3c(c2)C(c2ccccc2)(c2ccccc2)c2ccccc2-3)c2ccccc2-c2ccccc2)c([2H])c([2H])c1-c1ccc(-c2ccccc2)cc1]

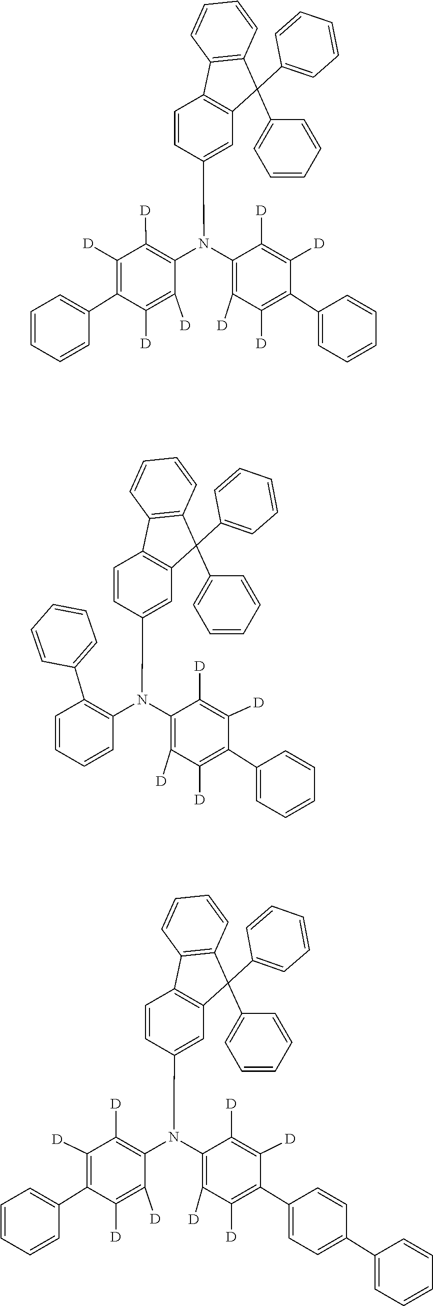 [SMILES: [2H]c1c([2H])c(N(c2ccc3c(c2)C(c2ccccc2)(c2ccccc2)c2ccccc2-3)c2c([2H])c([2H])c(-c3ccccc3)c([2H])c2[2H])c([2H])c([2H])c1-c1ccccc1 ; [2H]c1c([2H])c(N(c2ccc3c(c2)C(c2ccccc2)(c2ccccc2)c2ccccc2-3)c2ccccc2-c2ccccc2)c([2H])c([2H])c1-c1ccccc1 ; [2H]c1c([2H])c(N(c2ccc3c(c2)C(c2ccccc2)(c2ccccc2)c2ccccc2-3)c2c([2H])c([2H])c(-c3ccc(-c4ccccc4)cc3)c([2H])c2[2H])c([2H])c([2H])c1-c1ccccc1]

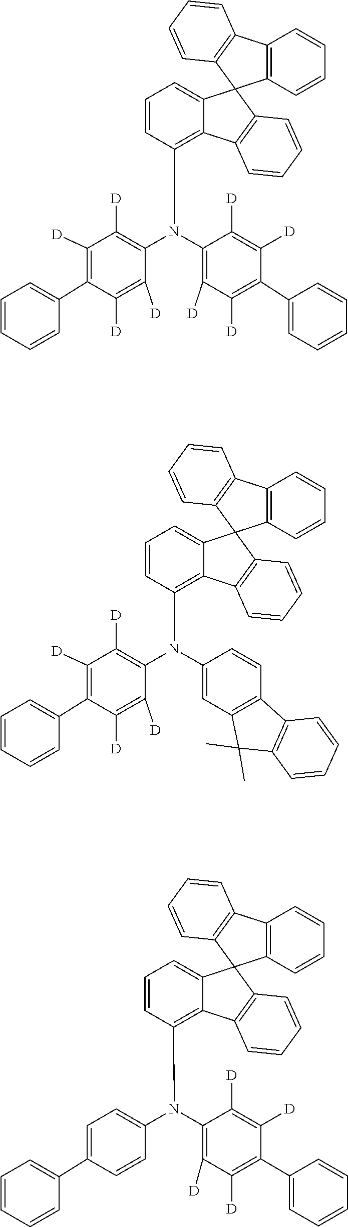 [SMILES: [2H]c1c([2H])c(N(c2cccc3c2-c2ccccc2C32c3ccccc3-c3ccccc32)c2c([2H])c([2H])c(-c3ccccc3)c([2H])c2[2H])c([2H])c([2H])c1-c1ccccc1 ; [2H]c1c([2H])c(N(c2ccc3c(c2)C(C)(C)c2ccccc2-3)c2cccc3c2-c2ccccc2C32c3ccccc3-c3ccccc32)c([2H])c([2H])c1-c1ccccc1 ; [2H]c1c([2H])c(N(c2ccc(-c3ccccc3)cc2)c2cccc3c2-c2ccccc2C32c3ccccc3-c3ccccc32)c([2H])c([2H])c1-c1ccccc1]

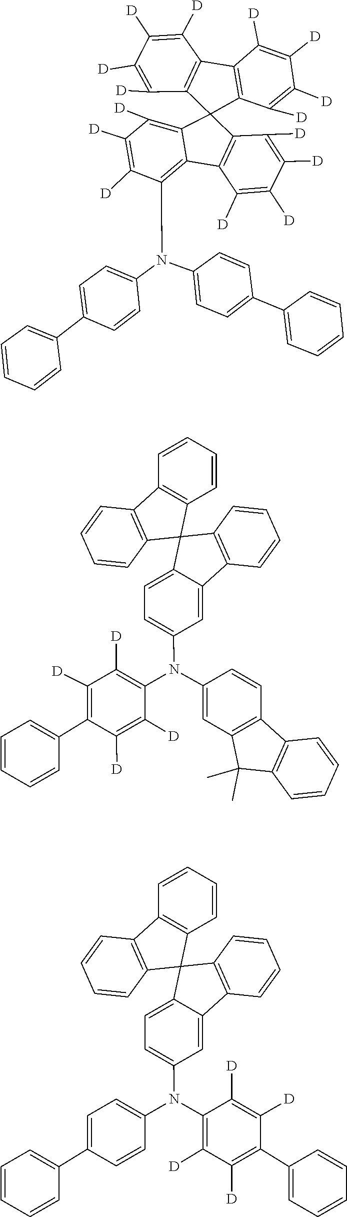 [SMILES: [2H]c1c([2H])c([2H])c2c(c1[2H])-c1c([2H])c([2H])c([2H])c([2H])c1C21c2c([2H])c([2H])c([2H])c([2H])c2-c2c(N(c3ccc(-c4ccccc4)cc3)c3ccc(-c4ccccc4)cc3)c([2H])c([2H])c([2H])c21 ; [2H]c1c([2H])c(N(c2ccc3c(c2)-c2ccccc2C32c3ccccc3-c3ccccc32)c2ccc3c(c2)C(C)(C)c2ccccc2-3)c([2H])c([2H])c1-c1ccccc1 ; [2H]c1c([2H])c(N(c2ccc(-c3ccccc3)cc2)c2ccc3c(c2)-c2ccccc2C32c3ccccc3-c3ccccc32)c([2H])c([2H])c1-c1ccccc1]

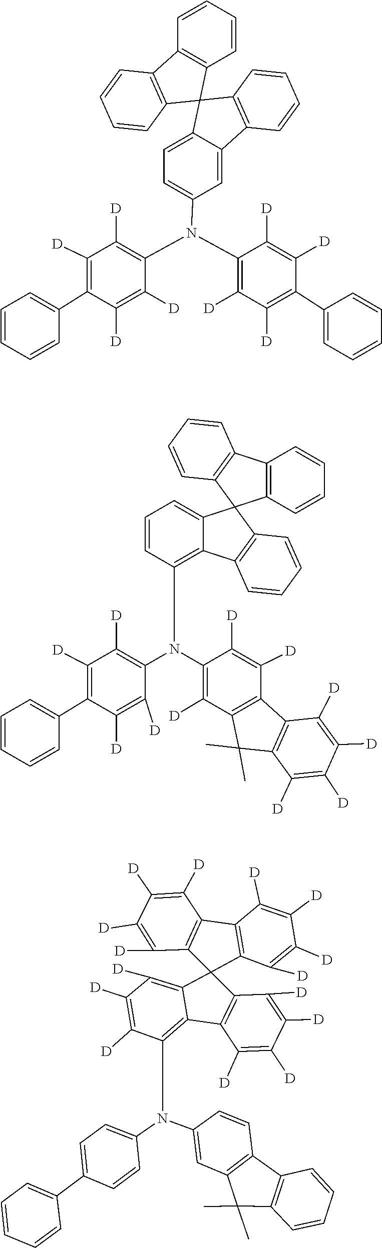 [SMILES: [2H]c1c([2H])c(N(c2ccc3c(c2)-c2ccccc2C32c3ccccc3-c3ccccc32)c2c([2H])c([2H])c(-c3ccccc3)c([2H])c2[2H])c([2H])c([2H])c1-c1ccccc1 ; [2H]c1c([2H])c([2H])c2c(c1[2H])-c1c([2H])c([2H])c(N(c3cccc4c3-c3ccccc3C43c4ccccc4-c4ccccc43)c3c([2H])c([2H])c(-c4ccccc4)c([2H])c3[2H])c([2H])c1C2(C)C ; [2H]c1c([2H])c([2H])c2c(c1[2H])-c1c([2H])c([2H])c([2H])c([2H])c1C21c2c([2H])c([2H])c([2H])c([2H])c2-c2c(N(c3ccc(-c4ccccc4)cc3)c3ccc4c(c3)C(C)(C)c3ccccc3-4)c([2H])c([2H])c([2H])c21]

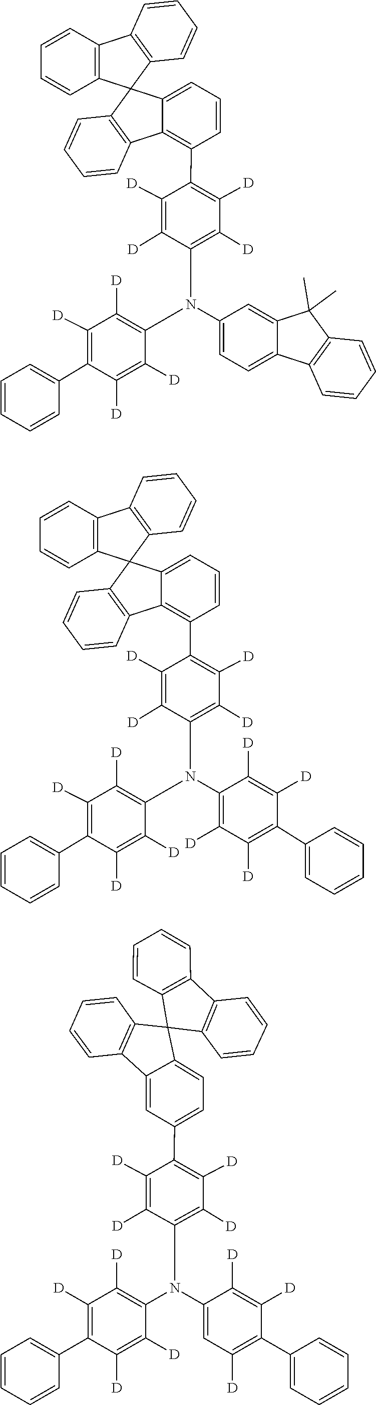 [SMILES: [2H]c1c([2H])c(N(c2ccc3c(c2)C(C)(C)c2ccccc2-3)c2c([2H])c([2H])c(-c3cccc4c3-c3ccccc3C43c4ccccc4-c4ccccc43)c([2H])c2[2H])c([2H])c([2H])c1-c1ccccc1 ; [2H]c1c([2H])c(N(c2c([2H])c([2H])c(-c3ccccc3)c([2H])c2[2H])c2c([2H])c([2H])c(-c3cccc4c3-c3ccccc3C43c4ccccc4-c4ccccc43)c([2H])c2[2H])c([2H])c([2H])c1-c1ccccc1 ; [2H]c1cc(N(c2c([2H])c([2H])c(-c3ccccc3)c([2H])c2[2H])c2c([2H])c([2H])c(-c3ccc4c(c3)-c3ccccc3C43c4ccccc4-c4ccccc43)c([2H])c2[2H])c([2H])c([2H])c1-c1ccccc1]

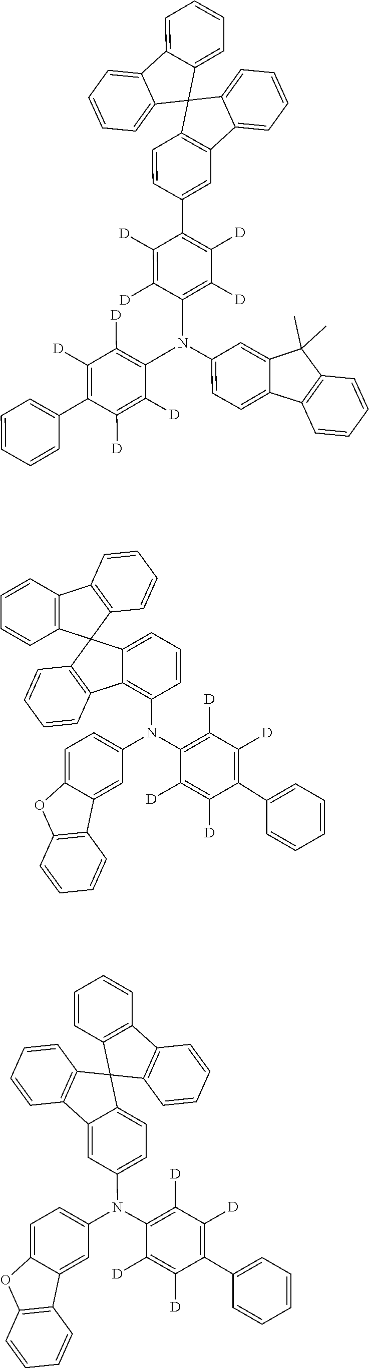 [SMILES: [2H]c1c([2H])c(N(c2ccc3c(c2)C(C)(C)c2ccccc2-3)c2c([2H])c([2H])c(-c3ccc4c(c3)-c3ccccc3C43c4ccccc4-c4ccccc43)c([2H])c2[2H])c([2H])c([2H])c1-c1ccccc1 ; [2H]c1c([2H])c(N(c2ccc3oc4ccccc4c3c2)c2cccc3c2-c2ccccc2C32c3ccccc3-c3ccccc32)c([2H])c([2H])c1-c1ccccc1 ; [2H]c1c([2H])c(N(c2ccc3c(c2)-c2ccccc2C32c3ccccc3-c3ccccc32)c2ccc3oc4ccccc4c3c2)c([2H])c([2H])c1-c1ccccc1]

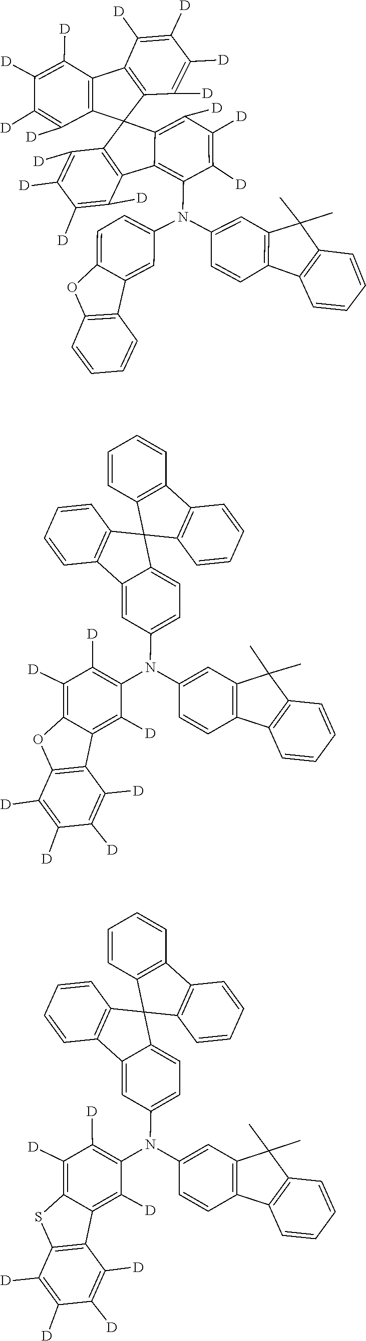 [SMILES: [2H]c1c([2H])c([2H])c2c(c1[2H])-c1c([2H])c([2H])c([2H])c([2H])c1C21c2c([2H])c([2H])c([2H])c([2H])c2-c2c(N(c3ccc4c(c3)C(C)(C)c3ccccc3-4)c3ccc4oc5ccccc5c4c3)c([2H])c([2H])c([2H])c21 ; [2H]c1c([2H])c([2H])c2c(oc3c([2H])c([2H])c(N(c4ccc5c(c4)-c4ccccc4C54c5ccccc5-c5ccccc54)c4ccc5c(c4)C(C)(C)c4ccccc4-5)c([2H])c32)c1[2H] ; [2H]c1c([2H])c([2H])c2c(sc3c([2H])c([2H])c(N(c4ccc5c(c4)-c4ccccc4C54c5ccccc5-c5ccccc54)c4ccc5c(c4)C(C)(C)c4ccccc4-5)c([2H])c32)c1[2H]]

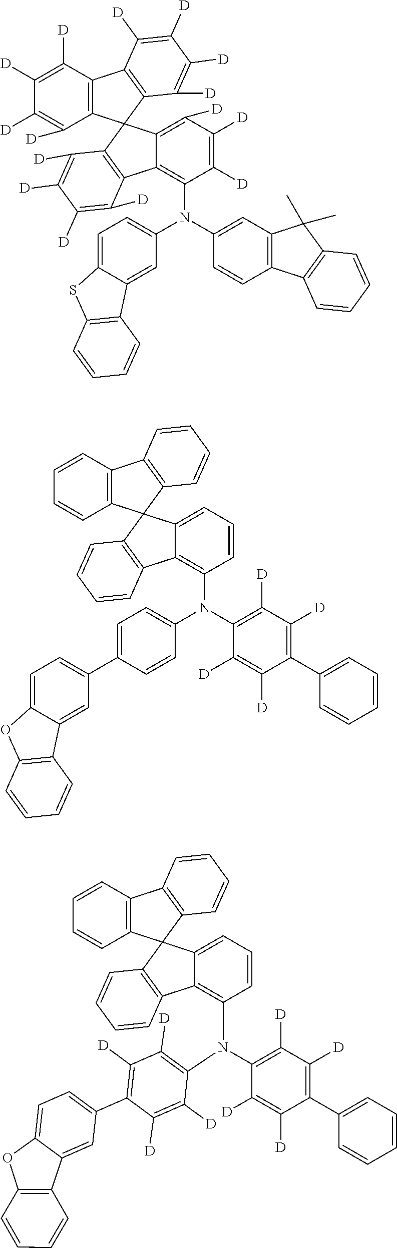 [SMILES: [2H]c1c([2H])c([2H])c2c(c1[2H])-c1c([2H])c([2H])c([2H])c([2H])c1C21c2c([2H])c([2H])c([2H])c([2H])c2-c2c(N(c3ccc4c(c3)C(C)(C)c3ccccc3-4)c3ccc4sc5ccccc5c4c3)c([2H])c([2H])c([2H])c21 ; [2H]c1c([2H])c(N(c2ccc(-c3ccc4oc5ccccc5c4c3)cc2)c2cccc3c2-c2ccccc2C32c3ccccc3-c3ccccc32)c([2H])c([2H])c1-c1ccccc1 ; [2H]c1c([2H])c(N(c2cccc3c2-c2ccccc2C32c3ccccc3-c3ccccc32)c2c([2H])c([2H])c(-c3ccc4oc5ccccc5c4c3)c([2H])c2[2H])c([2H])c([2H])c1-c1ccccc1]

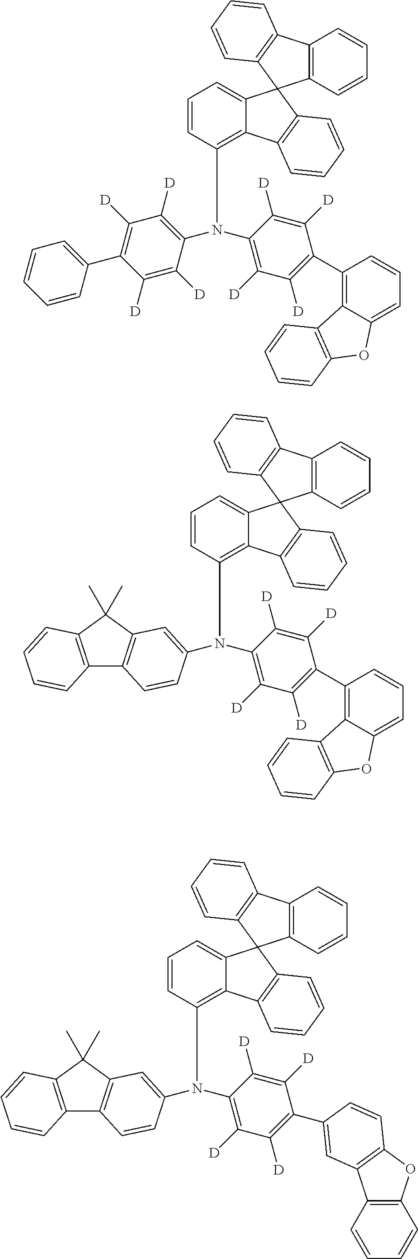

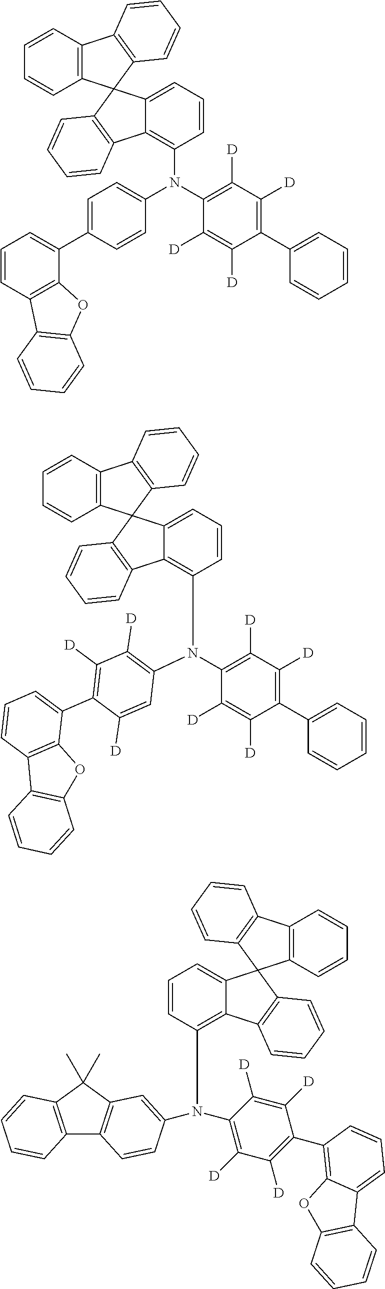 [SMILES: [2H]c1c([2H])c(N(c2ccc(-c3cccc4c3oc3ccccc34)cc2)c2cccc3c2-c2ccccc2C32c3ccccc3-c3ccccc32)c([2H])c([2H])c1-c1ccccc1 ; [2H]c1cc(N(c2cccc3c2-c2ccccc2C32c3ccccc3-c3ccccc32)c2c([2H])c([2H])c(-c3ccccc3)c([2H])c2[2H])c([2H])c([2H])c1-c1cccc2c1oc1ccccc12 ; [2H]c1c([2H])c(N(c2ccc3c(c2)C(C)(C)c2ccccc2-3)c2cccc3c2-c2ccccc2C32c3ccccc3-c3ccccc32)c([2H])c([2H])c1-c1cccc2c1oc1ccccc12]

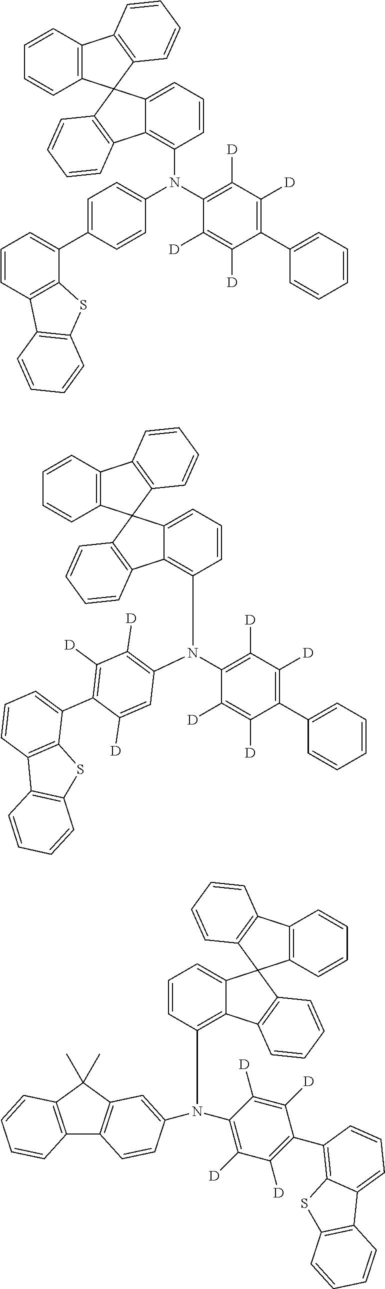 [SMILES: [2H]c1c([2H])c(N(c2ccc(-c3cccc4c3sc3ccccc34)cc2)c2cccc3c2-c2ccccc2C32c3ccccc3-c3ccccc32)c([2H])c([2H])c1-c1ccccc1 ; [2H]c1cc(N(c2cccc3c2-c2ccccc2C32c3ccccc3-c3ccccc32)c2c([2H])c([2H])c(-c3ccccc3)c([2H])c2[2H])c([2H])c([2H])c1-c1cccc2c1sc1ccccc12 ; [2H]c1c([2H])c(N(c2ccc3c(c2)C(C)(C)c2ccccc2-3)c2cccc3c2-c2ccccc2C32c3ccccc3-c3ccccc32)c([2H])c([2H])c1-c1cccc2c1sc1ccccc12]

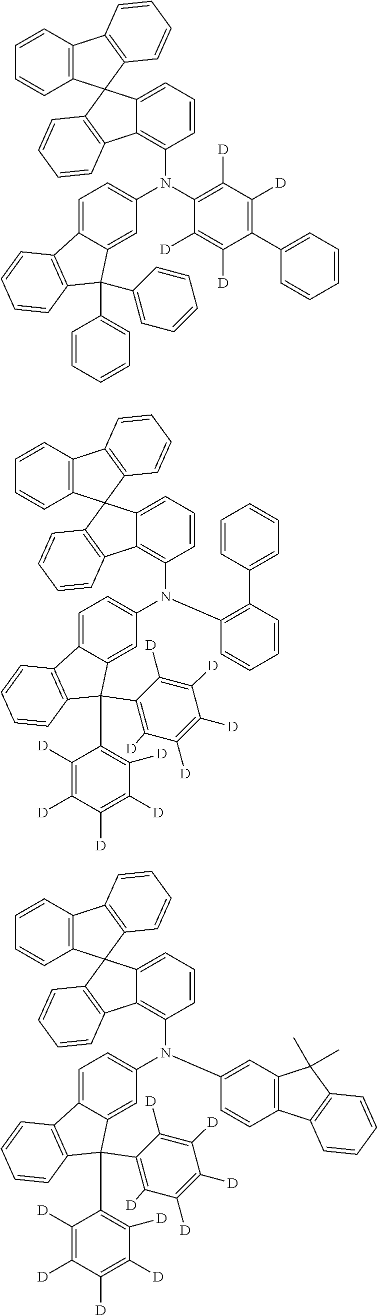

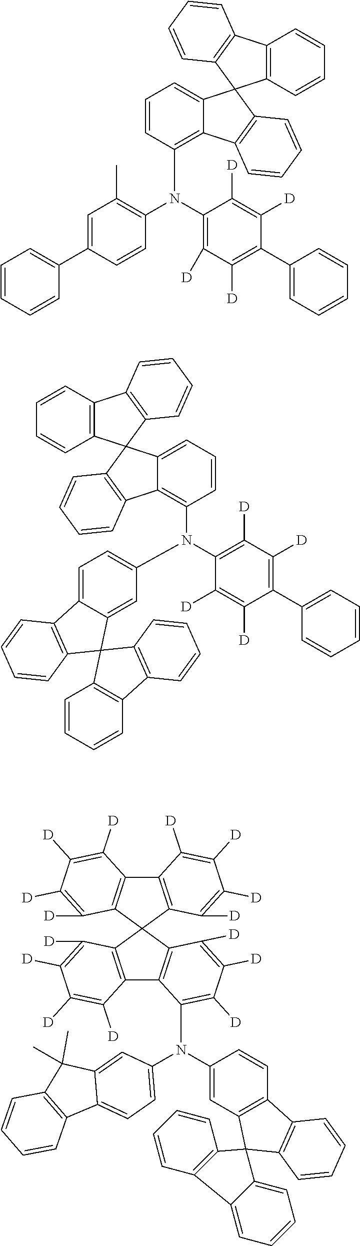 [SMILES: [2H]c1c([2H])c(N(c2ccc(-c3ccccc3)cc2C)c2cccc3c2-c2ccccc2C32c3ccccc3-c3ccccc32)c([2H])c([2H])c1-c1ccccc1 ; [2H]c1c([2H])c(N(c2ccc3c(c2)C2(c4ccccc4-c4ccccc42)c2ccccc2-3)c2cccc3c2-c2ccccc2C32c3ccccc3-c3ccccc32)c([2H])c([2H])c1-c1ccccc1 ; [2H]c1c([2H])c([2H])c2c(c1[2H])-c1c([2H])c([2H])c([2H])c([2H])c1C21c2c([2H])c([2H])c([2H])c([2H])c2-c2c(N(c3ccc4c(c3)C(C)(C)c3ccccc3-4)c3ccc4c(c3)C3(c5ccccc5-c5ccccc53)c3ccccc3-4)c([2H])c([2H])c([2H])c21]

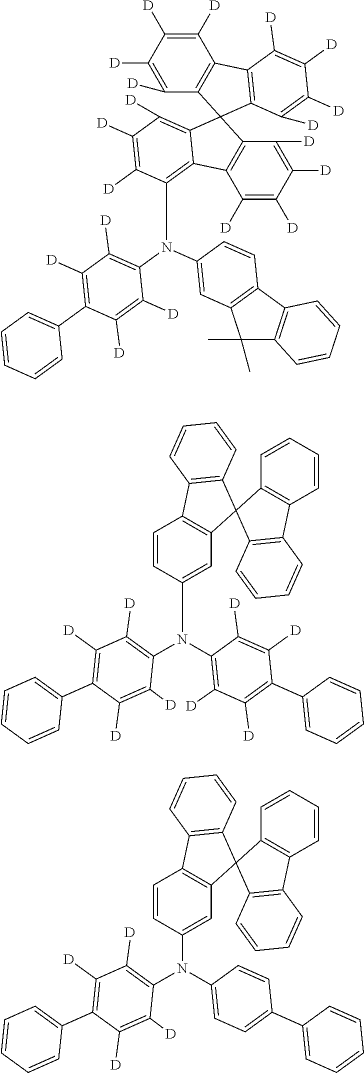 [SMILES: [2H]c1c([2H])c([2H])c2c(c1[2H])-c1c([2H])c([2H])c([2H])c([2H])c1C21c2c([2H])c([2H])c([2H])c([2H])c2-c2c(N(c3ccc4c(c3)C(C)(C)c3ccccc3-4)c3c([2H])c([2H])c(-c4ccccc4)c([2H])c3[2H])c([2H])c([2H])c([2H])c21 ; [2H]c1c([2H])c(N(c2ccc3c(c2)C2(c4ccccc4-c4ccccc42)c2ccccc2-3)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc3c(c2)C2(c4ccccc4-c4ccccc42)c2ccccc2-3)c([2H])c([2H])c1-c1ccccc1]

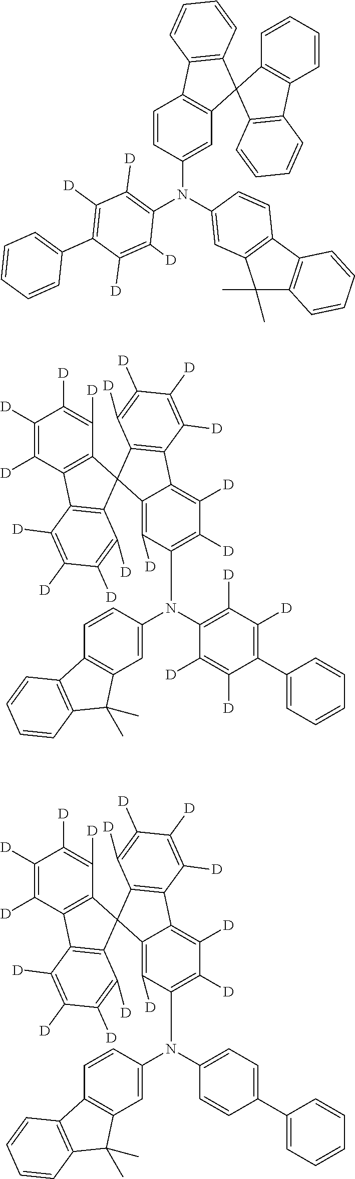

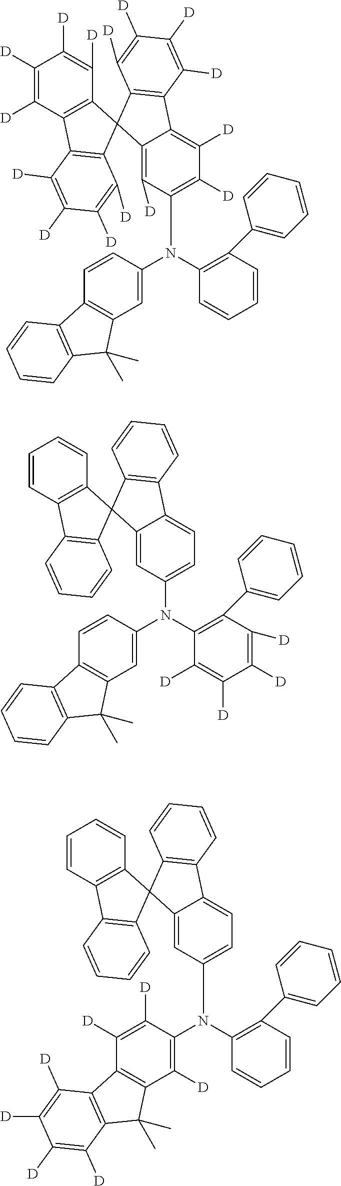

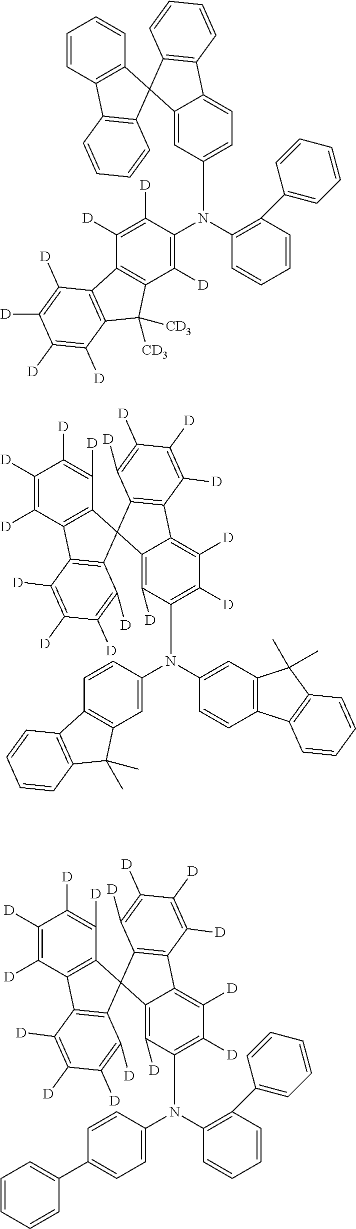

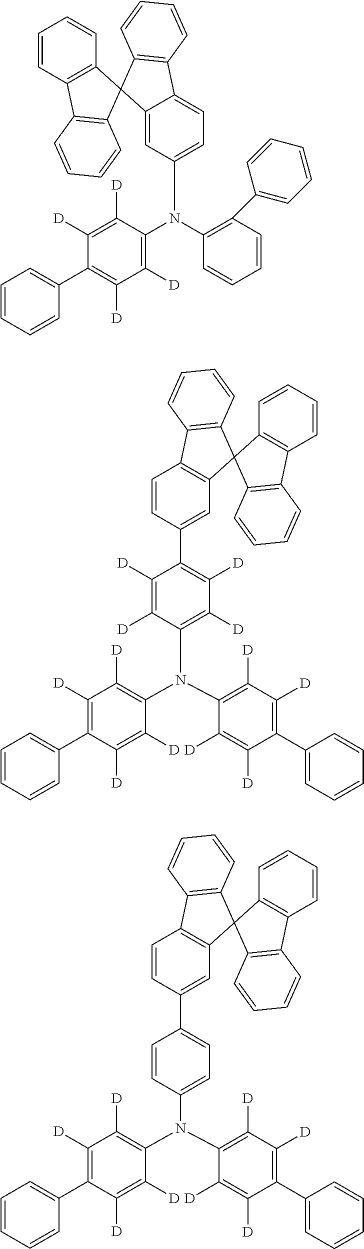 [SMILES: [2H]c1c([2H])c(N(c2ccc3c(c2)C2(c4ccccc4-c4ccccc42)c2ccccc2-3)c2ccccc2-c2ccccc2)c([2H])c([2H])c1-c1ccccc1 ; [2H]c1c([2H])c(N(c2c([2H])c([2H])c(-c3ccccc3)c([2H])c2[2H])c2c([2H])c([2H])c(-c3ccc4c(c3)C3(c5ccccc5-c5ccccc53)c3ccccc3-4)c([2H])c2[2H])c([2H])c([2H])c1-c1ccccc1 ; [2H]c1c([2H])c(N(c2ccc(-c3ccc4c(c3)C3(c5ccccc5-c5ccccc53)c3ccccc3-4)cc2)c2c([2H])c([2H])c(-c3ccccc3)c([2H])c2[2H])c([2H])c([2H])c1-c1ccccc1]

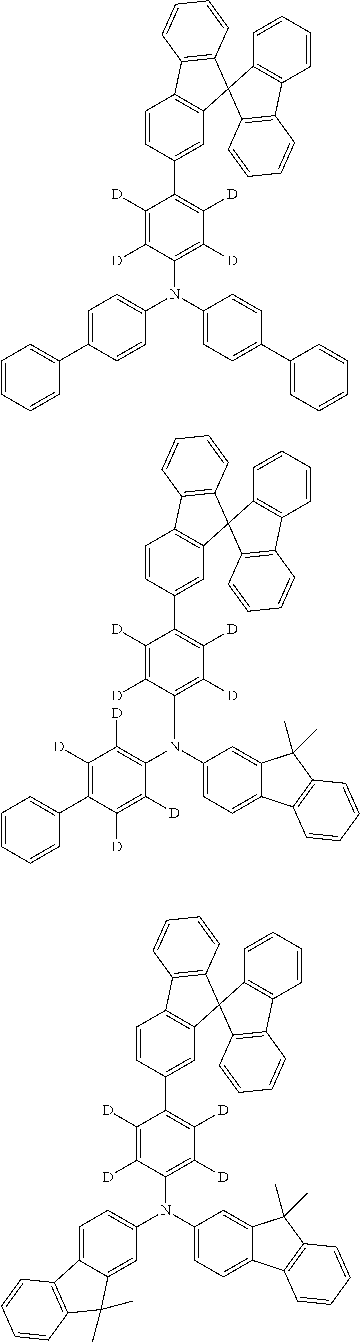 [SMILES: [2H]c1c([2H])c(N(c2ccc(-c3ccccc3)cc2)c2ccc(-c3ccccc3)cc2)c([2H])c([2H])c1-c1ccc2c(c1)C1(c3ccccc3-c3ccccc31)c1ccccc1-2 ; [2H]c1c([2H])c(N(c2ccc3c(c2)C(C)(C)c2ccccc2-3)c2c([2H])c([2H])c(-c3ccc4c(c3)C3(c5ccccc5-c5ccccc53)c3ccccc3-4)c([2H])c2[2H])c([2H])c([2H])c1-c1ccccc1 ; [2H]c1c([2H])c(N(c2ccc3c(c2)C(C)(C)c2ccccc2-3)c2ccc3c(c2)C(C)(C)c2ccccc2-3)c([2H])c([2H])c1-c1ccc2c(c1)C1(c3ccccc3-c3ccccc31)c1ccccc1-2]

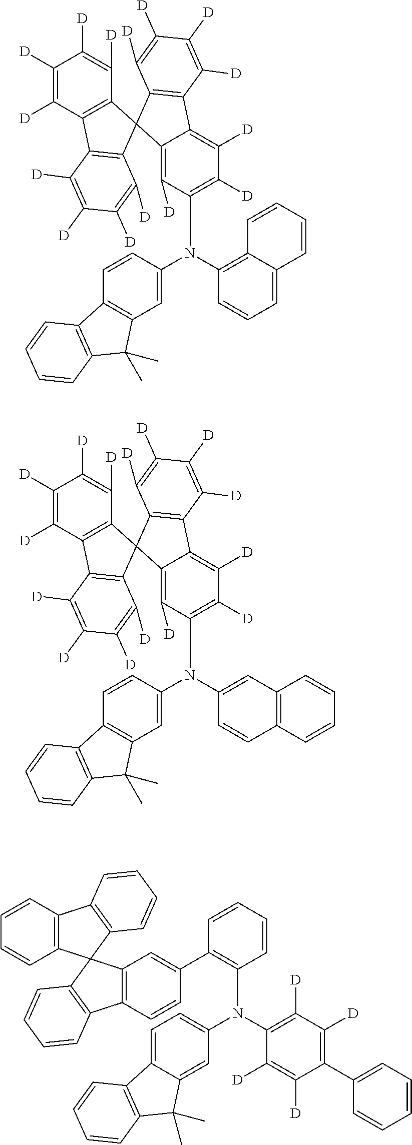 [SMILES: [2H]c1c([2H])c([2H])c2c(c1[2H])-c1c([2H])c([2H])c([2H])c([2H])c1C21c2c([2H])c([2H])c([2H])c([2H])c2-c2c([2H])c([2H])c(N(c3ccc4c(c3)C(C)(C)c3ccccc3-4)c3cccc4ccccc34)c([2H])c21 ; [2H]c1c([2H])c([2H])c2c(c1[2H])-c1c([2H])c([2H])c([2H])c([2H])c1C21c2c([2H])c([2H])c([2H])c([2H])c2-c2c([2H])c([2H])c(N(c3ccc4c(c3)C(C)(C)c3ccccc3-4)c3ccc4ccccc4c3)c([2H])c21 ; [2H]c1c([2H])c(N(c2ccc3c(c2)C(C)(C)c2ccccc2-3)c2ccccc2-c2ccc3c(c2)C2(c4ccccc4-c4ccccc42)c2ccccc2-3)c([2H])c([2H])c1-c1ccccc1]

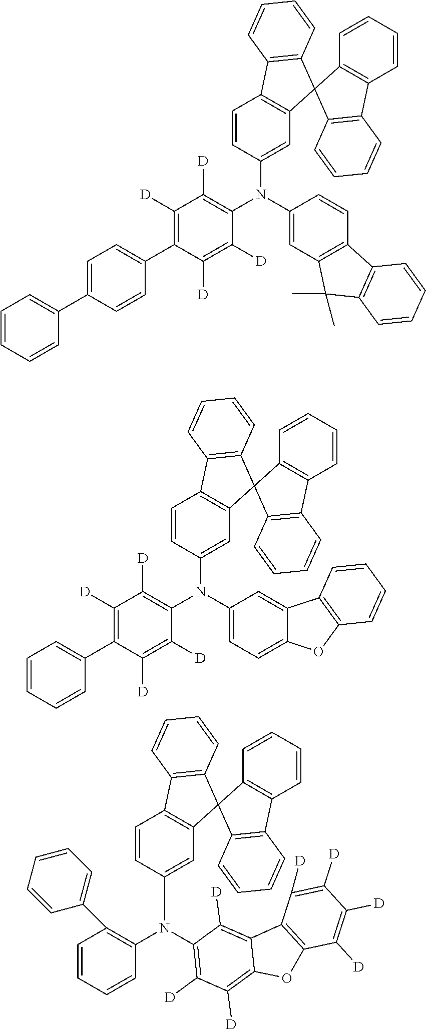

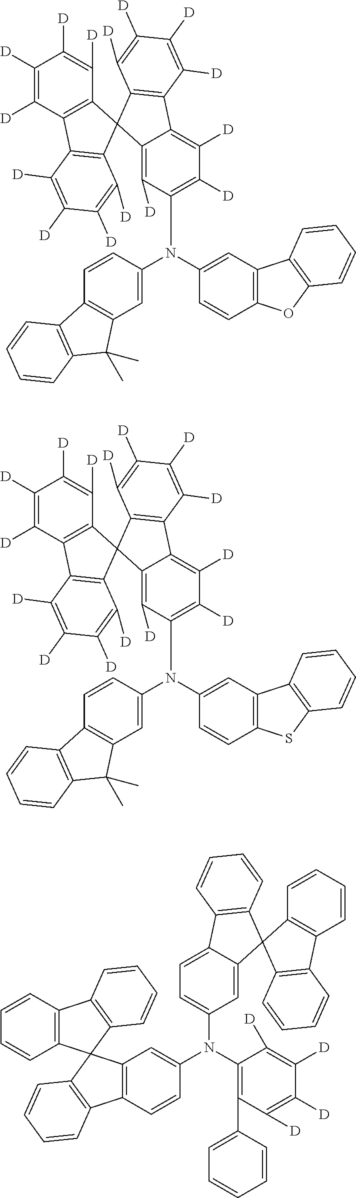 [SMILES: [2H]c1c([2H])c([2H])c2c(c1[2H])-c1c([2H])c([2H])c([2H])c([2H])c1C21c2c([2H])c([2H])c([2H])c([2H])c2-c2c([2H])c([2H])c(N(c3ccc4c(c3)C(C)(C)c3ccccc3-4)c3ccc4oc5ccccc5c4c3)c([2H])c21 ; [2H]c1c([2H])c([2H])c2c(c1[2H])-c1c([2H])c([2H])c([2H])c([2H])c1C21c2c([2H])c([2H])c([2H])c([2H])c2-c2c([2H])c([2H])c(N(c3ccc4c(c3)C(C)(C)c3ccccc3-4)c3ccc4sc5ccccc5c4c3)c([2H])c21 ; [2H]c1c([2H])c([2H])c(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccccc2)c1[2H]]

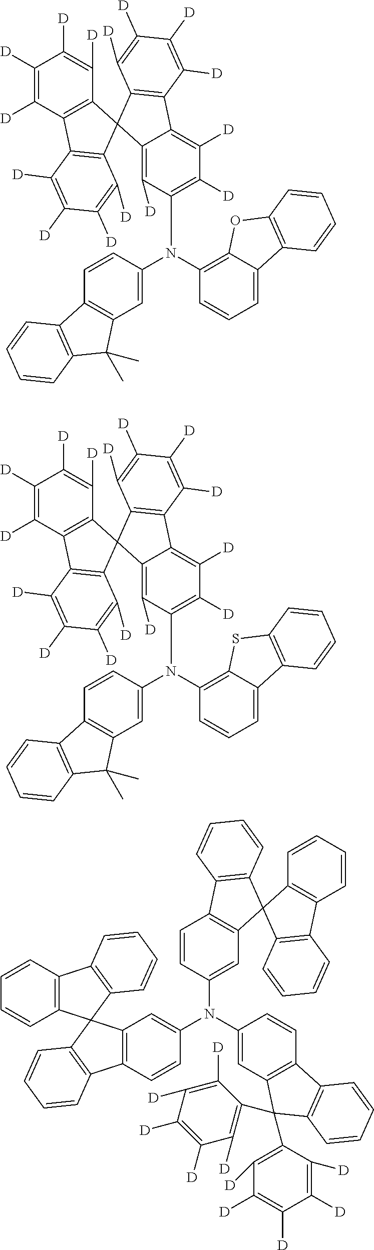

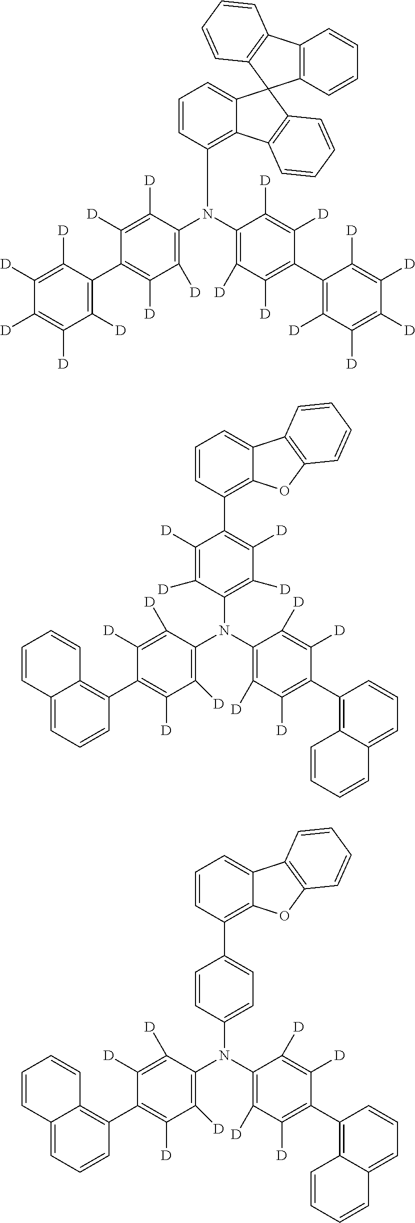

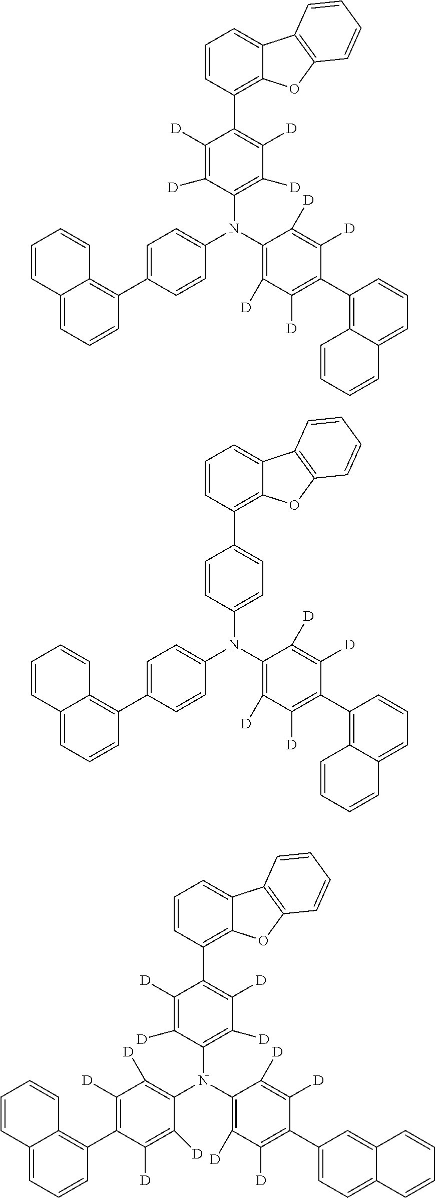

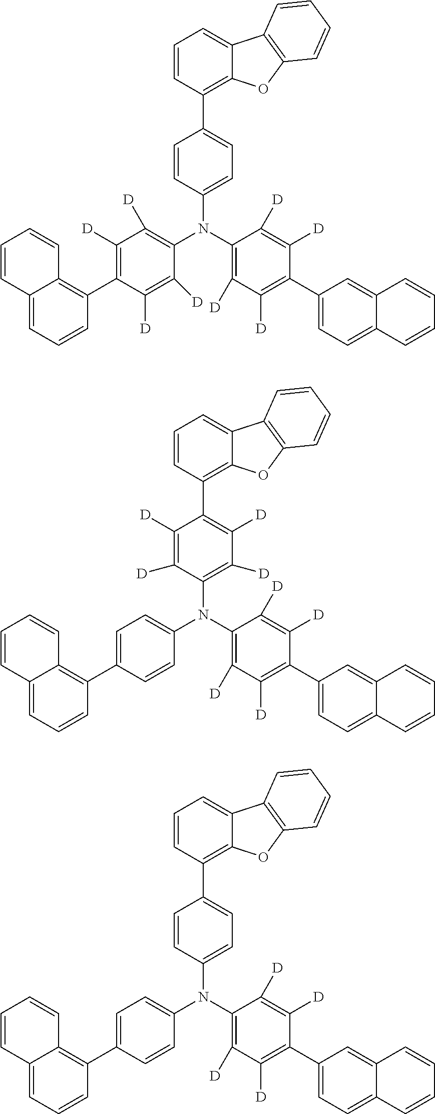 [SMILES: [2H]c1c([2H])c(N(c2ccc(-c3cccc4c3oc3ccccc34)cc2)c2c([2H])c([2H])c(-c3cccc4ccccc34)c([2H])c2[2H])c([2H])c([2H])c1-c1ccc2ccccc2c1 ; [2H]c1c([2H])c(N(c2ccc(-c3cccc4ccccc34)cc2)c2c([2H])c([2H])c(-c3cccc4c3oc3ccccc34)c([2H])c2[2H])c([2H])c([2H])c1-c1ccc2ccccc2c1 ; [2H]c1c([2H])c(N(c2ccc(-c3cccc4ccccc34)cc2)c2ccc(-c3cccc4c3oc3ccccc34)cc2)c([2H])c([2H])c1-c1ccc2ccccc2c1]

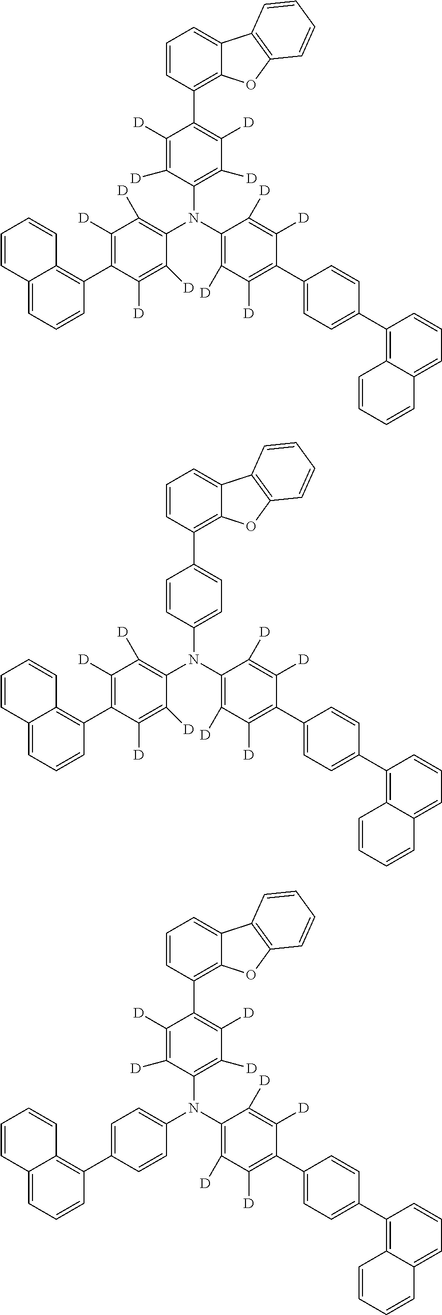 [SMILES: [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1ccc(-c2cccc3ccccc23)cc1 ; [2H]c1c([2H])c(N(c2ccc(-c3cccc4c3oc3ccccc34)cc2)c2c([2H])c([2H])c(-c3cccc4ccccc34)c([2H])c2[2H])c([2H])c([2H])c1-c1ccc(-c2cccc3ccccc23)cc1 ; [2H]c1c([2H])c(N(c2ccc(-c3cccc4ccccc34)cc2)c2c([2H])c([2H])c(-c3cccc4c3oc3ccccc34)c([2H])c2[2H])c([2H])c([2H])c1-c1ccc(-c2cccc3ccccc23)cc1]

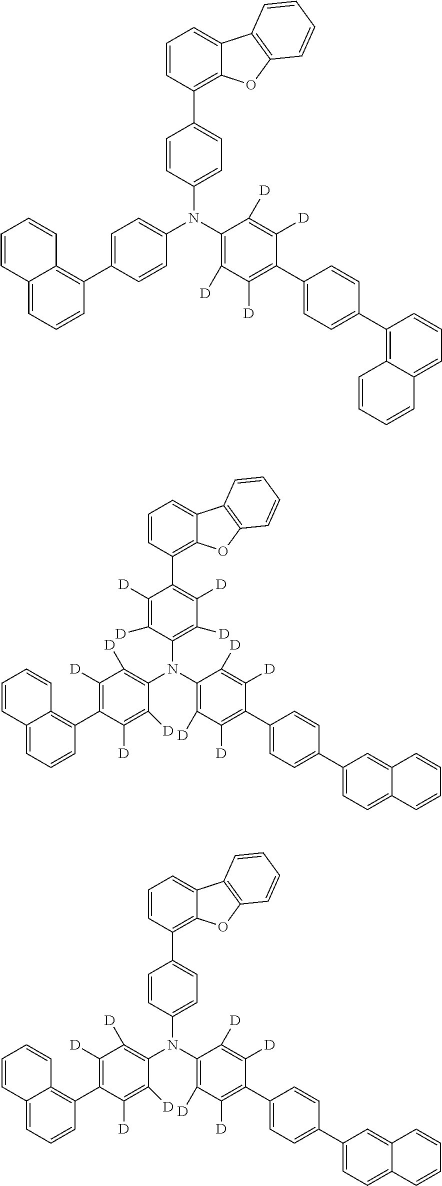 [SMILES: [2H]c1c([2H])c(N(c2ccc(-c3cccc4ccccc34)cc2)c2ccc(-c3cccc4c3oc3ccccc34)cc2)c([2H])c([2H])c1-c1ccc(-c2cccc3ccccc23)cc1 ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1ccc(-c2ccc3ccccc3c2)cc1 ; [2H]c1c([2H])c(N(c2ccc(-c3cccc4c3oc3ccccc34)cc2)c2c([2H])c([2H])c(-c3cccc4ccccc34)c([2H])c2[2H])c([2H])c([2H])c1-c1ccc(-c2ccc3ccccc3c2)cc1]

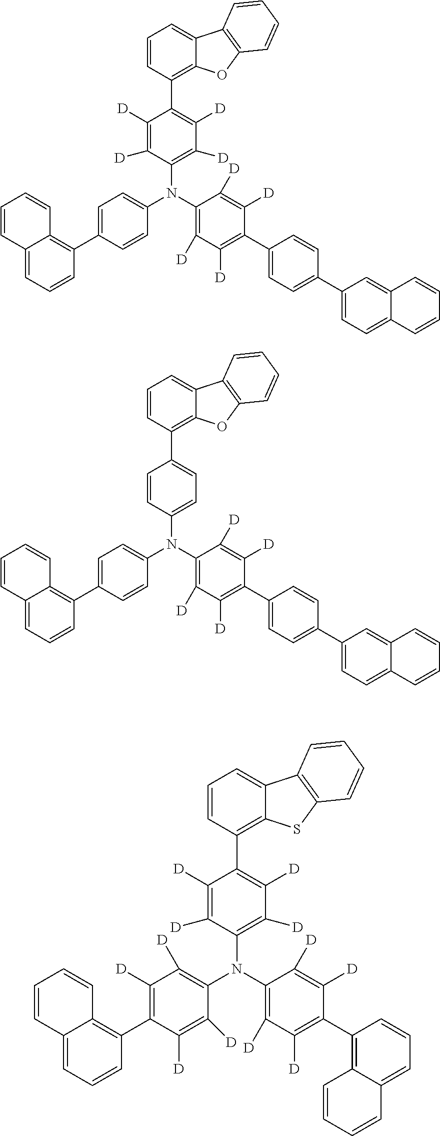 [SMILES: [2H]c1c([2H])c(N(c2ccc(-c3cccc4ccccc34)cc2)c2c([2H])c([2H])c(-c3cccc4c3oc3ccccc34)c([2H])c2[2H])c([2H])c([2H])c1-c1ccc(-c2ccc3ccccc3c2)cc1 ; [2H]c1c([2H])c(N(c2ccc(-c3cccc4ccccc34)cc2)c2ccc(-c3cccc4c3oc3ccccc34)cc2)c([2H])c([2H])c1-c1ccc(-c2ccc3ccccc3c2)cc1 ; [2H]c1c([2H])c(N(c2c([2H])c([2H])c(-c3cccc4ccccc34)c([2H])c2[2H])c2c([2H])c([2H])c(-c3cccc4c3sc3ccccc34)c([2H])c2[2H])c([2H])c([2H])c1-c1cccc2ccccc12]

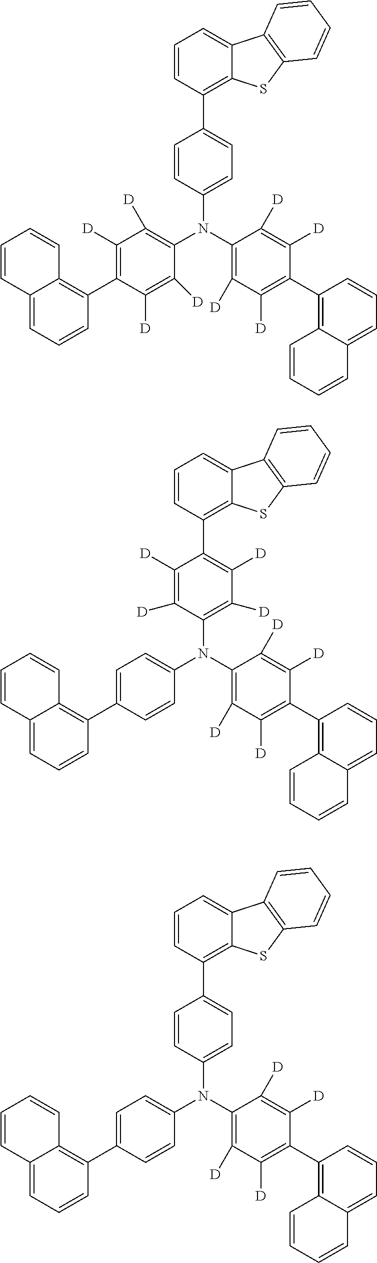

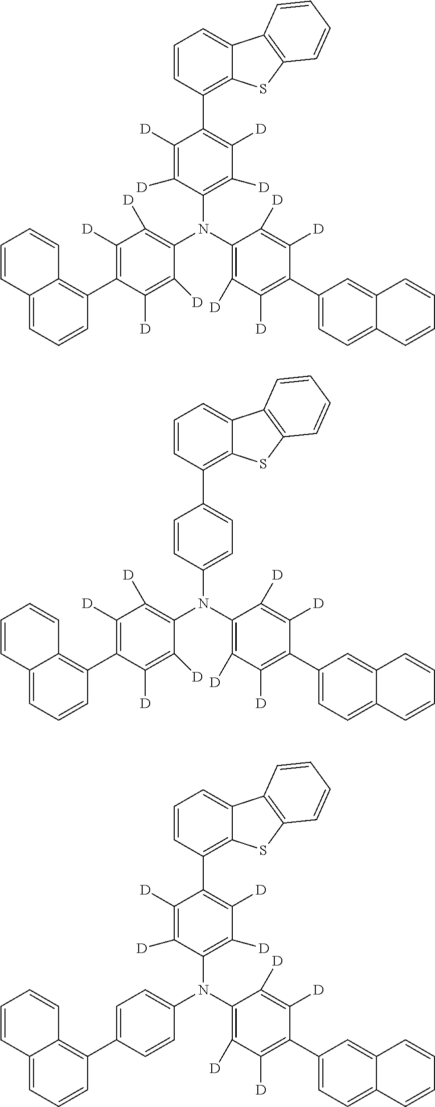 [SMILES: [2H]c1c([2H])c(N(c2c([2H])c([2H])c(-c3cccc4ccccc34)c([2H])c2[2H])c2c([2H])c([2H])c(-c3cccc4c3sc3ccccc34)c([2H])c2[2H])c([2H])c([2H])c1-c1ccc2ccccc2c1 ; [2H]c1c([2H])c(N(c2ccc(-c3cccc4c3sc3ccccc34)cc2)c2c([2H])c([2H])c(-c3cccc4ccccc34)c([2H])c2[2H])c([2H])c([2H])c1-c1ccc2ccccc2c1 ; [2H]c1c([2H])c(N(c2ccc(-c3cccc4ccccc34)cc2)c2c([2H])c([2H])c(-c3cccc4c3sc3ccccc34)c([2H])c2[2H])c([2H])c([2H])c1-c1ccc2ccccc2c1]

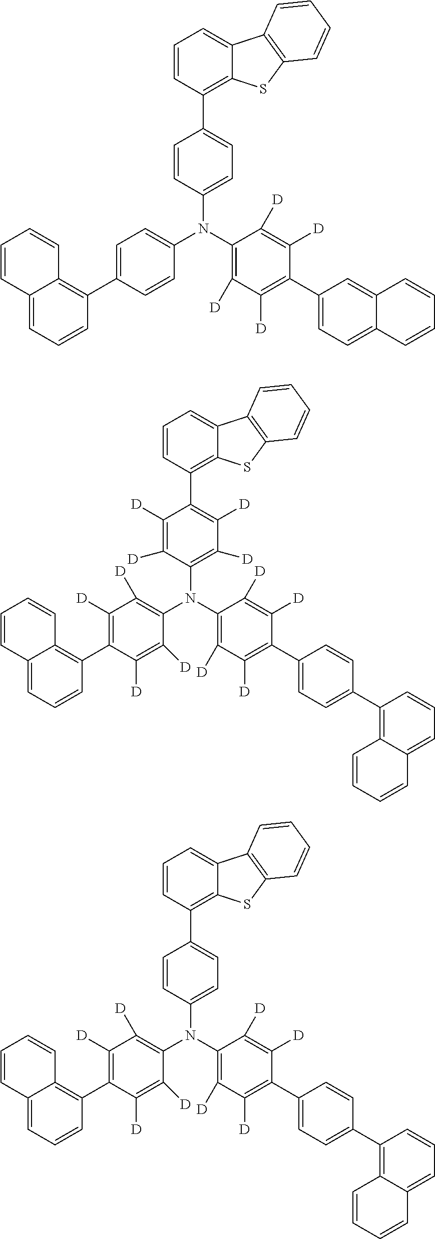 [SMILES: [2H]c1c([2H])c(N(c2ccc(-c3cccc4ccccc34)cc2)c2ccc(-c3cccc4c3sc3ccccc34)cc2)c([2H])c([2H])c1-c1ccc2ccccc2c1 ; [2H]c1c([2H])c(N(c2c([2H])c([2H])c(-c3cccc4ccccc34)c([2H])c2[2H])c2c([2H])c([2H])c(-c3cccc4c3sc3ccccc34)c([2H])c2[2H])c([2H])c([2H])c1-c1ccc(-c2cccc3ccccc23)cc1 ; [2H]c1c([2H])c(N(c2ccc(-c3cccc4c3sc3ccccc34)cc2)c2c([2H])c([2H])c(-c3cccc4ccccc34)c([2H])c2[2H])c([2H])c([2H])c1-c1ccc(-c2cccc3ccccc23)cc1]

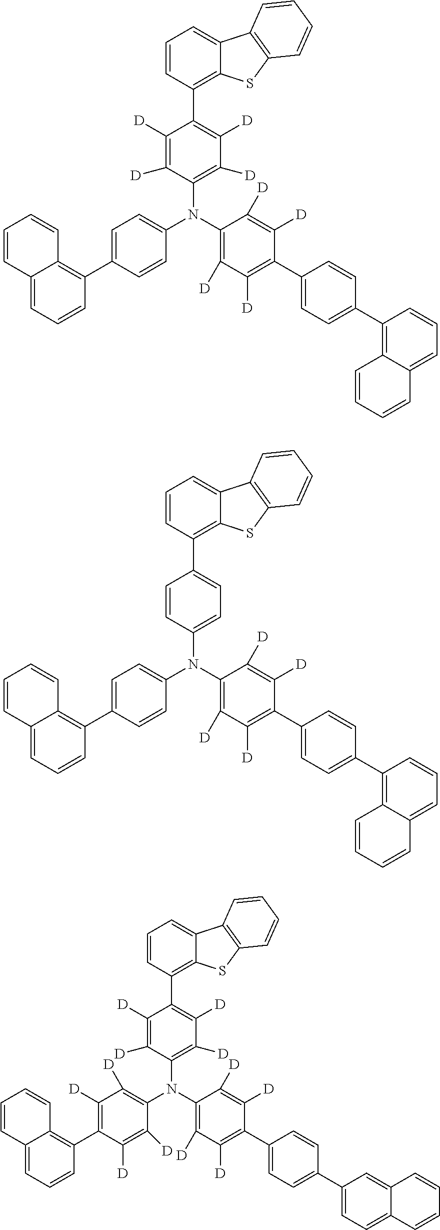 [SMILES: [2H]c1c([2H])c(N(c2ccc(-c3cccc4ccccc34)cc2)c2c([2H])c([2H])c(-c3cccc4c3sc3ccccc34)c([2H])c2[2H])c([2H])c([2H])c1-c1ccc(-c2cccc3ccccc23)cc1 ; [2H]c1c([2H])c(N(c2ccc(-c3cccc4ccccc34)cc2)c2ccc(-c3cccc4c3sc3ccccc34)cc2)c([2H])c([2H])c1-c1ccc(-c2cccc3ccccc23)cc1 ; [2H]c1c([2H])c(N(c2c([2H])c([2H])c(-c3cccc4ccccc34)c([2H])c2[2H])c2c([2H])c([2H])c(-c3cccc4c3sc3ccccc34)c([2H])c2[2H])c([2H])c([2H])c1-c1ccc(-c2ccc3ccccc3c2)cc1]

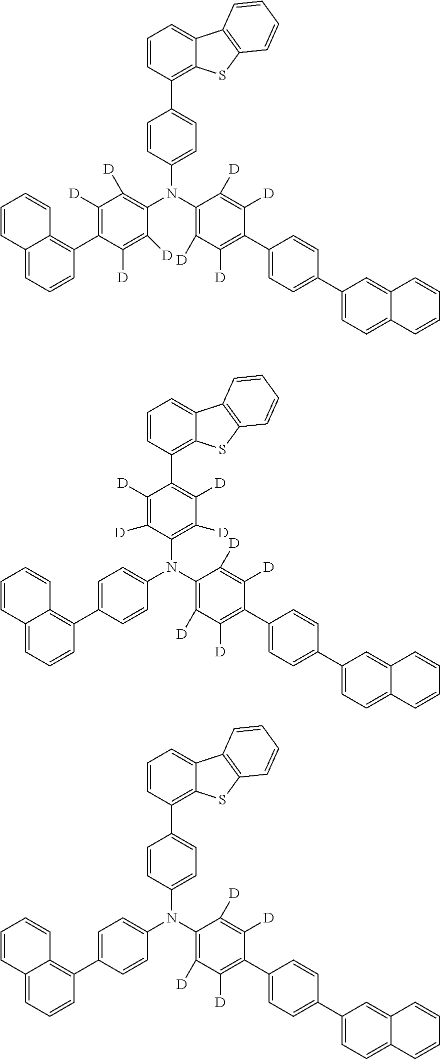 [SMILES: [2H]c1c([2H])c(N(c2ccc(-c3cccc4c3sc3ccccc34)cc2)c2c([2H])c([2H])c(-c3cccc4ccccc34)c([2H])c2[2H])c([2H])c([2H])c1-c1ccc(-c2ccc3ccccc3c2)cc1 ; [2H]c1c([2H])c(N(c2ccc(-c3cccc4ccccc34)cc2)c2c([2H])c([2H])c(-c3cccc4c3sc3ccccc34)c([2H])c2[2H])c([2H])c([2H])c1-c1ccc(-c2ccc3ccccc3c2)cc1 ; [2H]c1c([2H])c(N(c2ccc(-c3cccc4ccccc34)cc2)c2ccc(-c3cccc4c3sc3ccccc34)cc2)c([2H])c([2H])c1-c1ccc(-c2ccc3ccccc3c2)cc1]

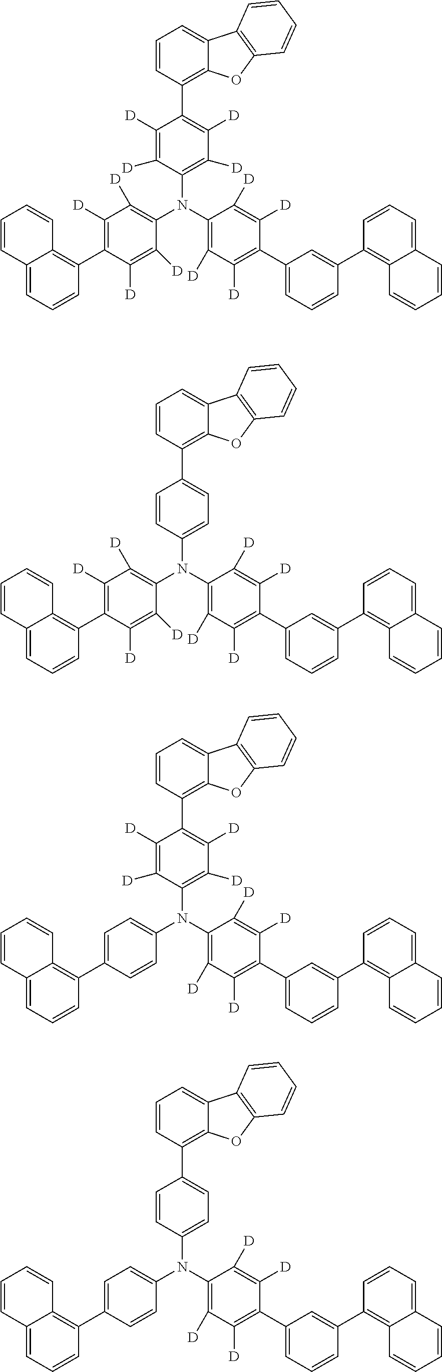

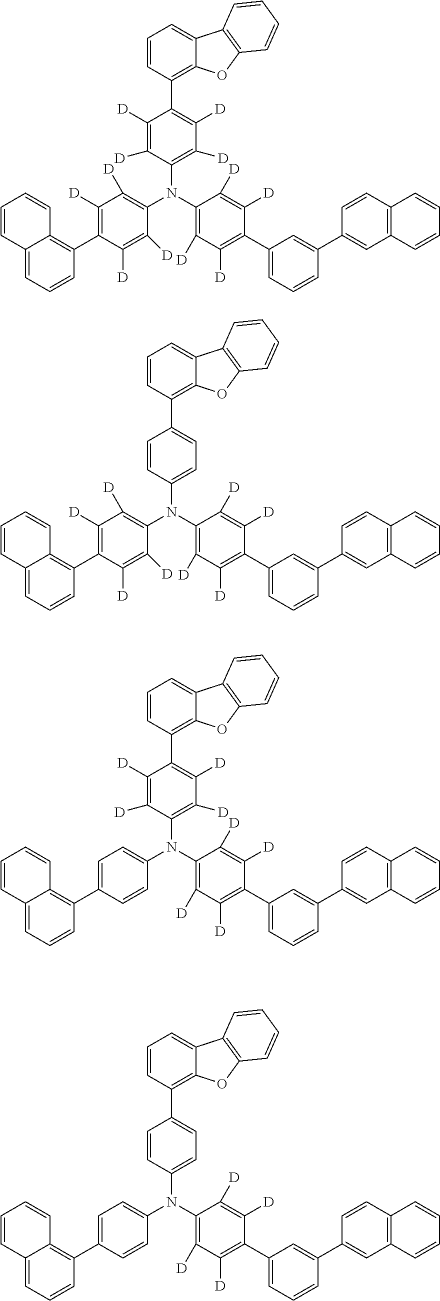

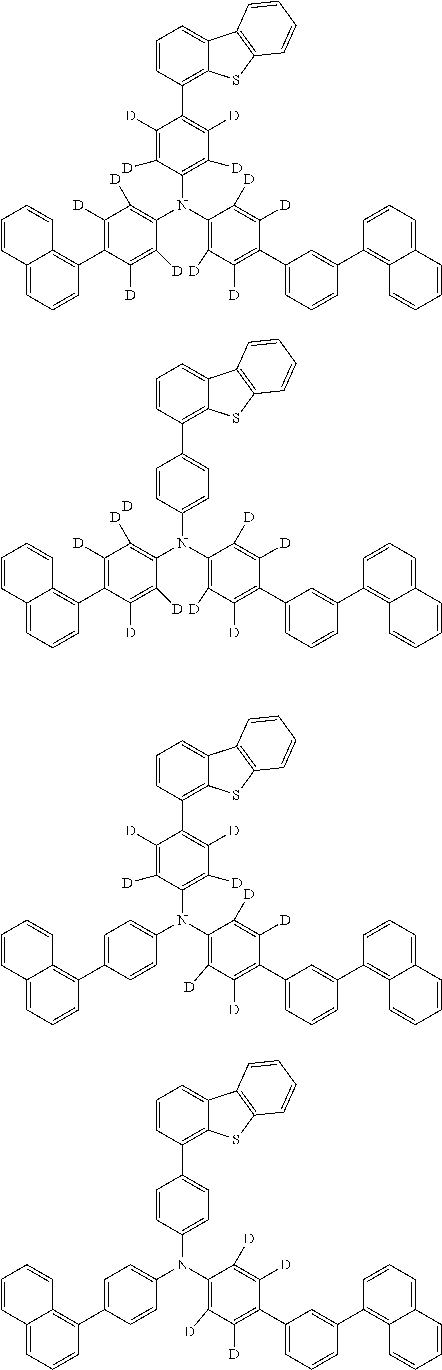

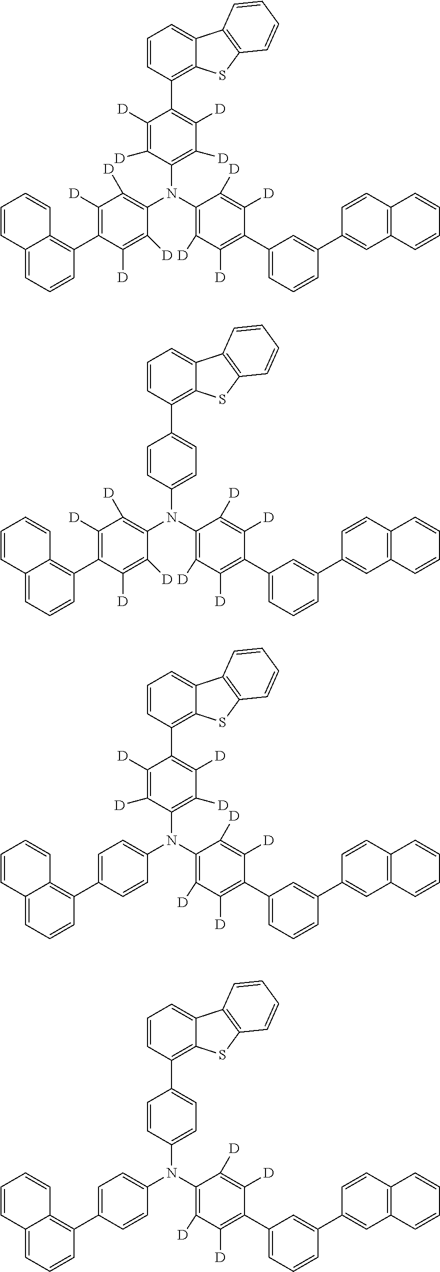

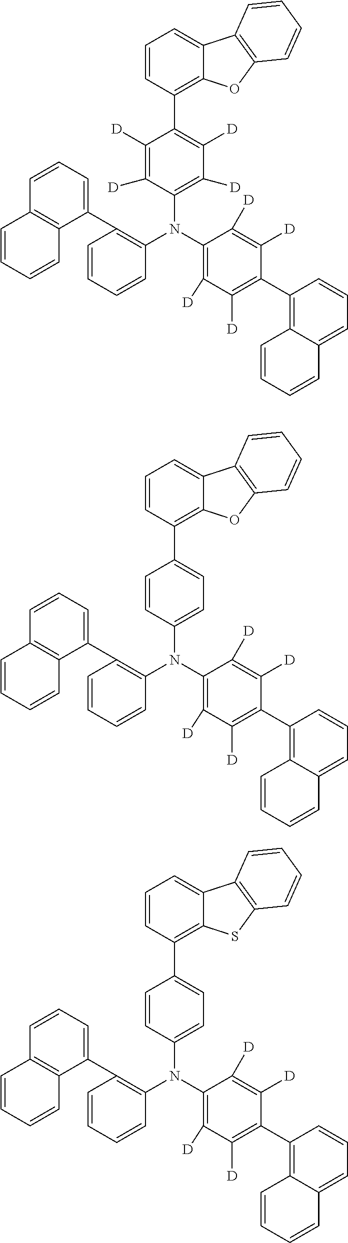

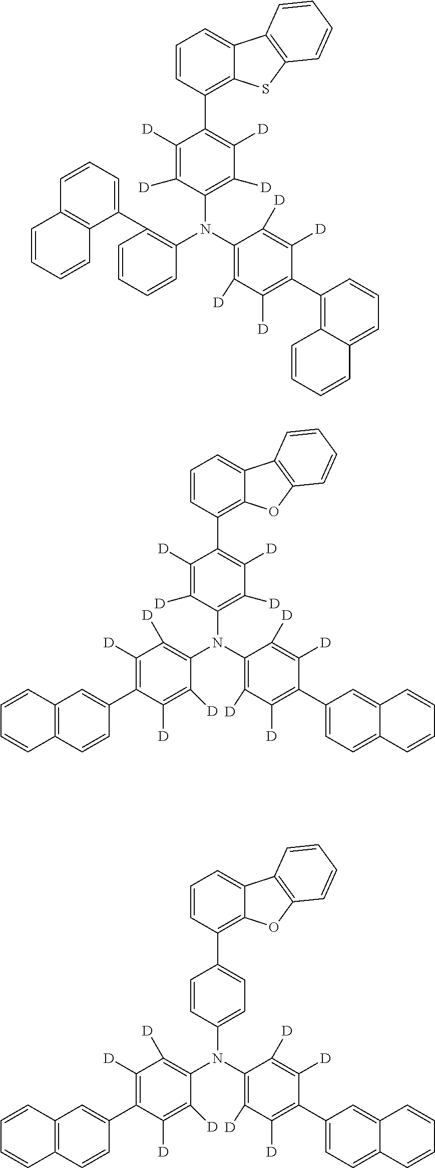

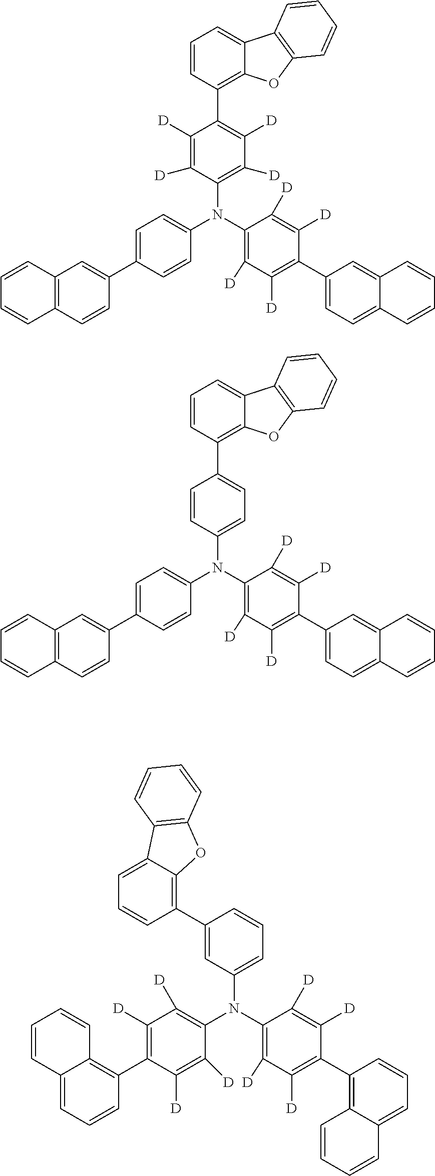 [SMILES: [2H]c1c([2H])c(N(c2ccc(-c3ccc4ccccc4c3)cc2)c2c([2H])c([2H])c(-c3cccc4c3oc3ccccc34)c([2H])c2[2H])c([2H])c([2H])c1-c1ccc2ccccc2c1 ; [2H]c1c([2H])c(N(c2ccc(-c3ccc4ccccc4c3)cc2)c2ccc(-c3cccc4c3oc3ccccc34)cc2)c([2H])c([2H])c1-c1ccc2ccccc2c1 ; [2H]c1c([2H])c(N(c2cccc(-c3cccc4c3oc3ccccc34)c2)c2c([2H])c([2H])c(-c3cccc4ccccc34)c([2H])c2[2H])c([2H])c([2H])c1-c1cccc2ccccc12]

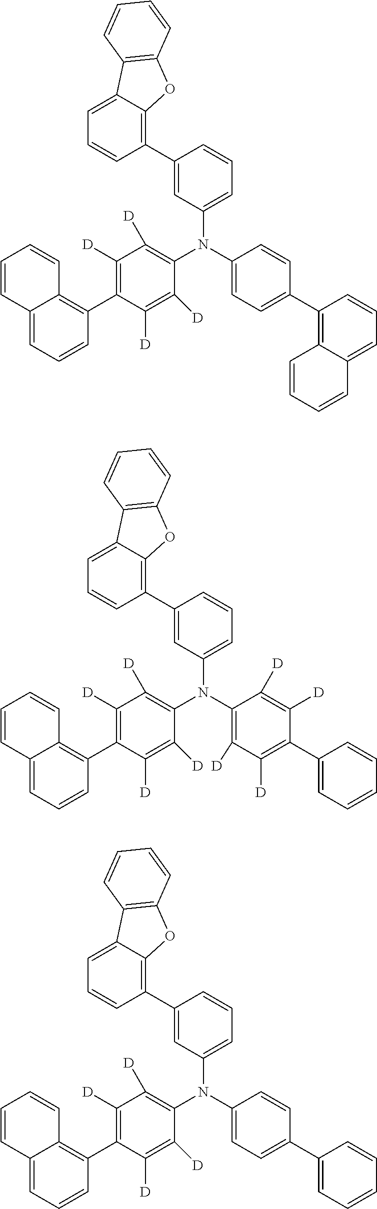 [SMILES: [2H]c1c([2H])c(N(c2ccc(-c3cccc4ccccc34)cc2)c2cccc(-c3cccc4c3oc3ccccc34)c2)c([2H])c([2H])c1-c1cccc2ccccc12 ; [2H]c1c([2H])c(N(c2cccc(-c3cccc4c3oc3ccccc34)c2)c2c([2H])c([2H])c(-c3cccc4ccccc34)c([2H])c2[2H])c([2H])c([2H])c1-c1ccccc1 ; [2H]c1c([2H])c(N(c2ccc(-c3ccccc3)cc2)c2cccc(-c3cccc4c3oc3ccccc34)c2)c([2H])c([2H])c1-c1cccc2ccccc12]

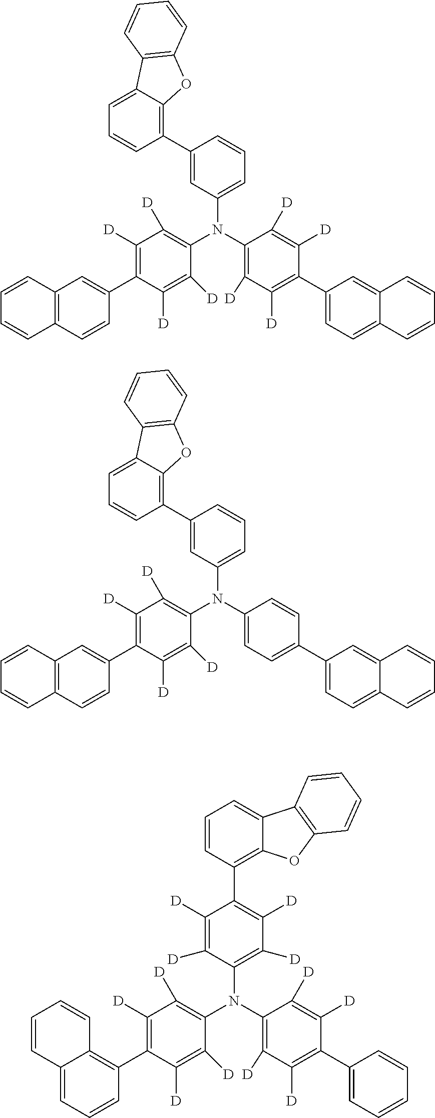 [SMILES: [2H]c1c([2H])c(N(c2cccc(-c3cccc4c3oc3ccccc34)c2)c2c([2H])c([2H])c(-c3ccc4ccccc4c3)c([2H])c2[2H])c([2H])c([2H])c1-c1ccc2ccccc2c1 ; [2H]c1c([2H])c(N(c2ccc(-c3ccc4ccccc4c3)cc2)c2cccc(-c3cccc4c3oc3ccccc34)c2)c([2H])c([2H])c1-c1ccc2ccccc2c1 ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1ccccc1]

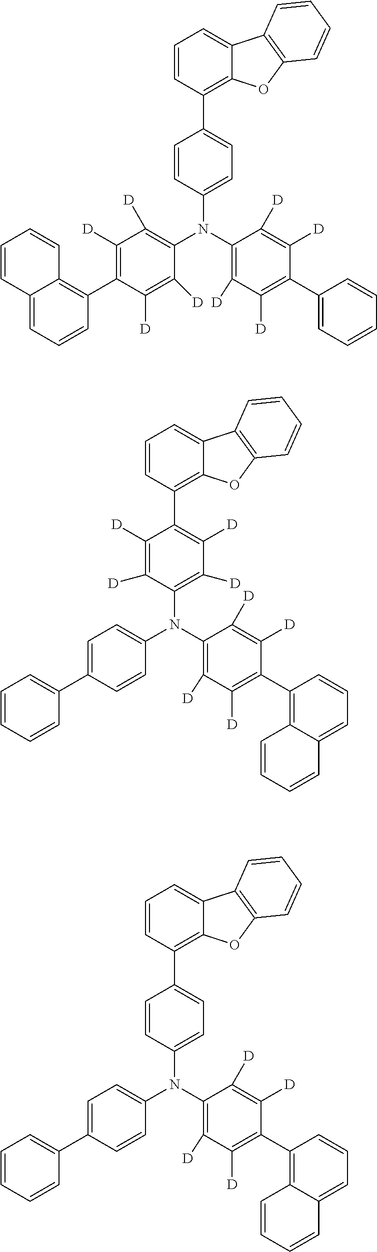 [SMILES: [2H]c1c([2H])c(N(c2ccc(-c3cccc4c3oc3ccccc34)cc2)c2c([2H])c([2H])c(-c3cccc4ccccc34)c([2H])c2[2H])c([2H])c([2H])c1-c1ccccc1 ; [2H]c1c([2H])c(N(c2ccc(-c3ccccc3)cc2)c2c([2H])c([2H])c(-c3cccc4c3oc3ccccc34)c([2H])c2[2H])c([2H])c([2H])c1-c1cccc2ccccc12 ; [2H]c1c([2H])c(N(c2ccc(-c3ccccc3)cc2)c2ccc(-c3cccc4c3oc3ccccc34)cc2)c([2H])c([2H])c1-c1cccc2ccccc12]

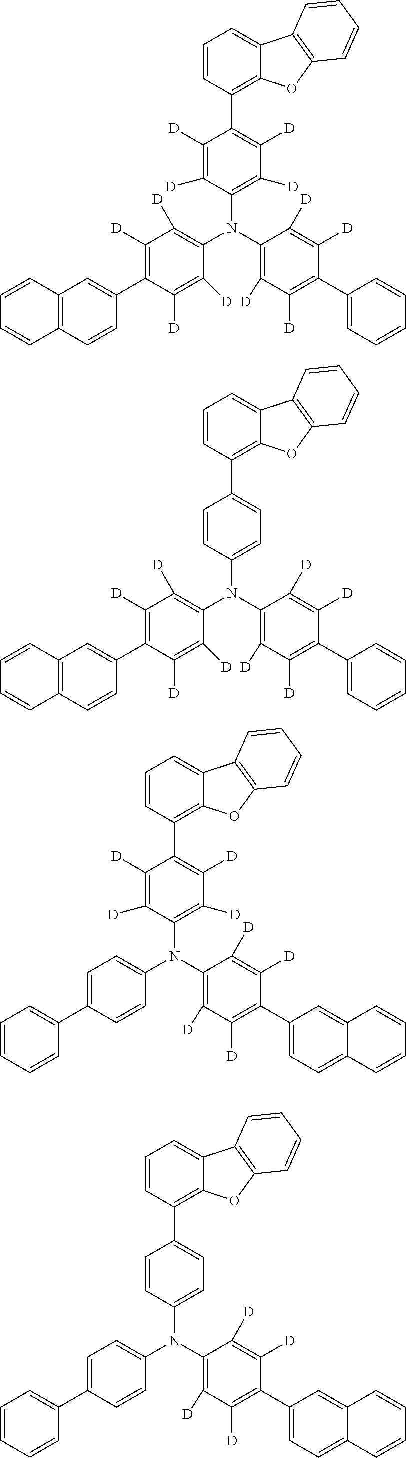

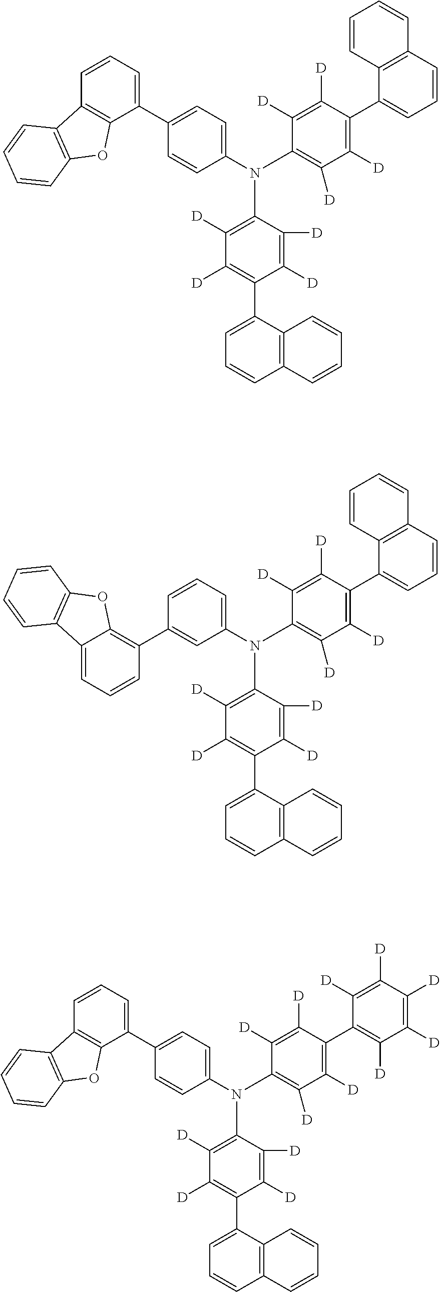 [SMILES: [2H]c1c([2H])c(N(c2ccc(-c3cccc4c3oc3ccccc34)cc2)c2c([2H])c([2H])c(-c3cccc4ccccc34)c([2H])c2[2H])c([2H])c([2H])c1-c1cccc2ccccc12 ; [2H]c1c([2H])c(N(c2cccc(-c3cccc4c3oc3ccccc34)c2)c2c([2H])c([2H])c(-c3cccc4ccccc34)c([2H])c2[2H])c([2H])c([2H])c1-c1cccc2ccccc12 ; [2H]c1c([2H])c([2H])c(-c2c([2H])c([2H])c(N(c3ccc(-c4cccc5c4oc4ccccc45)cc3)c3c([2H])c([2H])c(-c4cccc5ccccc45)c([2H])c3[2H])c([2H])c2[2H])c([2H])c1[2H]]

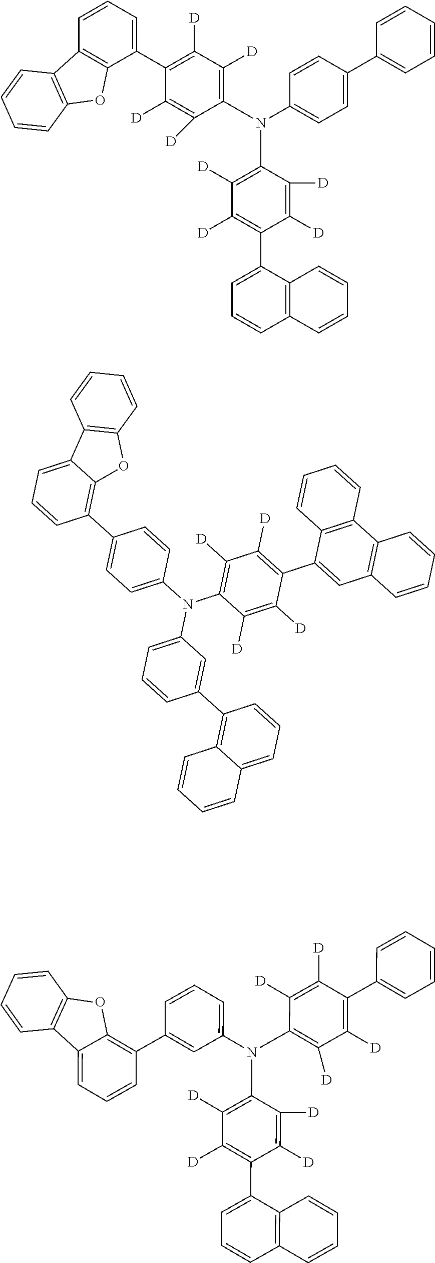 [SMILES: [2H]c1c([2H])c(N(c2ccc(-c3ccccc3)cc2)c2c([2H])c([2H])c(-c3cccc4c3oc3ccccc34)c([2H])c2[2H])c([2H])c([2H])c1-c1cccc2ccccc12 ; [2H]c1c([2H])c(N(c2ccc(-c3cccc4c3oc3ccccc34)cc2)c2cccc(-c3cccc4ccccc34)c2)c([2H])c([2H])c1-c1cc2ccccc2c2ccccc12 ; [2H]c1c([2H])c(N(c2cccc(-c3cccc4c3oc3ccccc34)c2)c2c([2H])c([2H])c(-c3cccc4ccccc34)c([2H])c2[2H])c([2H])c([2H])c1-c1ccccc1]

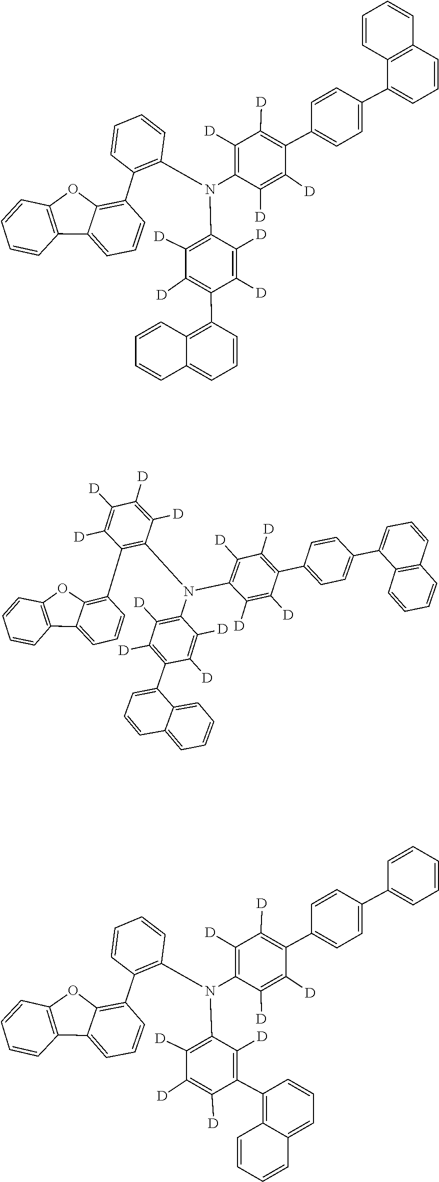 [SMILES: [2H]c1c([2H])c(N(c2ccccc2-c2cccc3c2oc2ccccc23)c2c([2H])c([2H])c(-c3cccc4ccccc34)c([2H])c2[2H])c([2H])c([2H])c1-c1ccc(-c2cccc3ccccc23)cc1 ; [2H]c1c([2H])c([2H])c(N(c2c([2H])c([2H])c(-c3ccc(-c4cccc5ccccc45)cc3)c([2H])c2[2H])c2c([2H])c([2H])c(-c3cccc4ccccc34)c([2H])c2[2H])c(-c2cccc3c2oc2ccccc23)c1[2H] ; [2H]c1c([2H])c(-c2cccc3ccccc23)c([2H])c(N(c2ccccc2-c2cccc3c2oc2ccccc23)c2c([2H])c([2H])c(-c3ccc(-c4ccccc4)cc3)c([2H])c2[2H])c1[2H]]

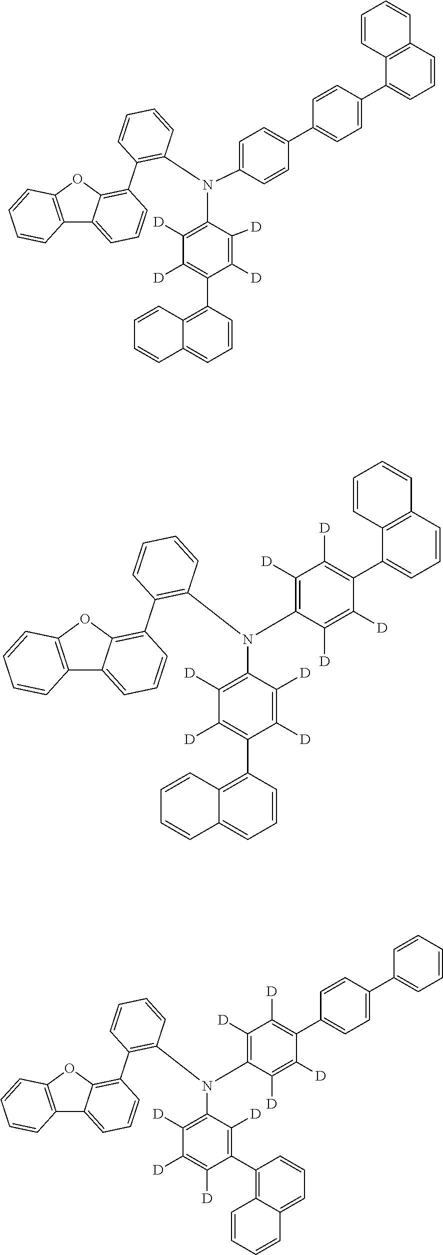 [SMILES: [2H]c1c([2H])c(N(c2ccc(-c3ccc(-c4cccc5ccccc45)cc3)cc2)c2ccccc2-c2cccc3c2oc2ccccc23)c([2H])c([2H])c1-c1cccc2ccccc12 ; [2H]c1c([2H])c(N(c2ccccc2-c2cccc3c2oc2ccccc23)c2c([2H])c([2H])c(-c3cccc4ccccc34)c([2H])c2[2H])c([2H])c([2H])c1-c1cccc2ccccc12 ; [2H]c1c([2H])c(-c2cccc3ccccc23)c([2H])c(N(c2ccccc2-c2cccc3c2oc2ccccc23)c2c([2H])c([2H])c(-c3ccc(-c4ccccc4)cc3)c([2H])c2[2H])c1[2H]]

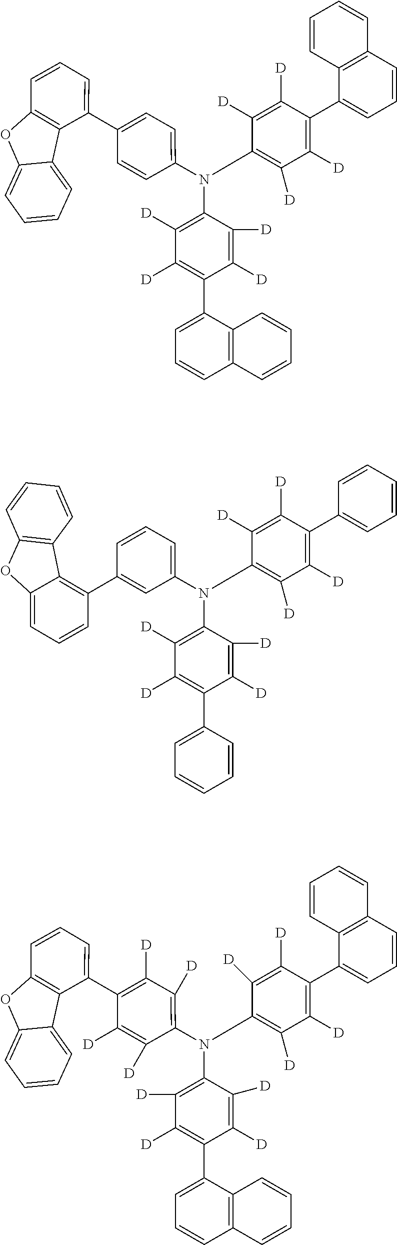 [SMILES: [2H]c1c([2H])c(N(c2ccc(-c3cccc4oc5ccccc5c34)cc2)c2c([2H])c([2H])c(-c3cccc4ccccc34)c([2H])c2[2H])c([2H])c([2H])c1-c1cccc2ccccc12 ; [2H]c1c([2H])c(N(c2cccc(-c3cccc4oc5ccccc5c34)c2)c2c([2H])c([2H])c(-c3ccccc3)c([2H])c2[2H])c([2H])c([2H])c1-c1ccccc1 ; [2H]c1c([2H])c(N(c2c([2H])c([2H])c(-c3cccc4ccccc34)c([2H])c2[2H])c2c([2H])c([2H])c(-c3cccc4oc5ccccc5c34)c([2H])c2[2H])c([2H])c([2H])c1-c1cccc2ccccc12]

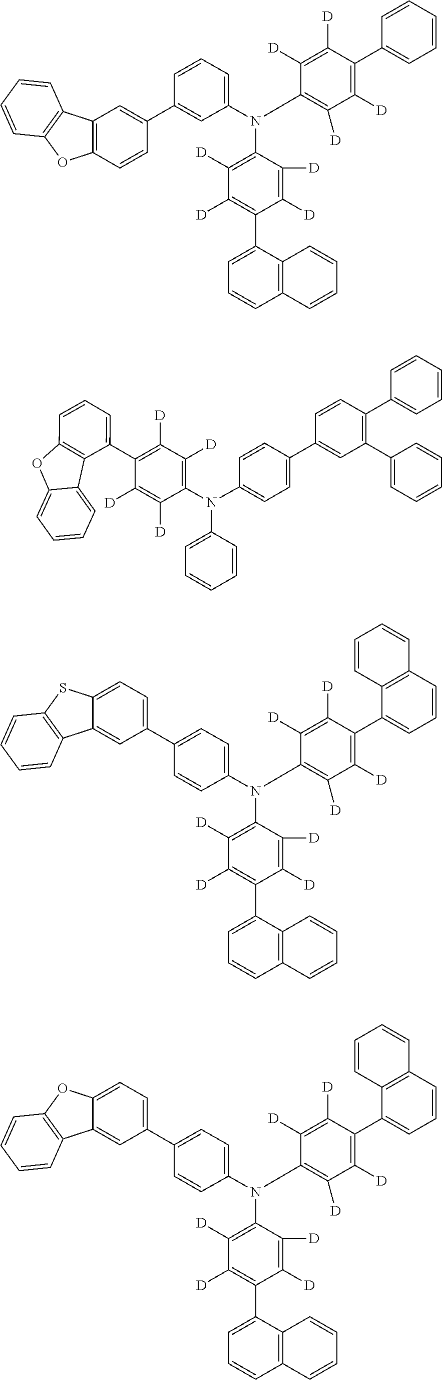 [SMILES: [2H]c1c([2H])c(N(c2cccc(-c3ccc4oc5ccccc5c4c3)c2)c2c([2H])c([2H])c(-c3cccc4ccccc34)c([2H])c2[2H])c([2H])c([2H])c1-c1ccccc1 ; [2H]c1c([2H])c(N(c2ccccc2)c2ccc(-c3ccc(-c4ccccc4)c(-c4ccccc4)c3)cc2)c([2H])c([2H])c1-c1cccc2oc3ccccc3c12 ; [2H]c1c([2H])c(N(c2ccc(-c3ccc4sc5ccccc5c4c3)cc2)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccc4oc5ccccc5c4c3)cc2)c2c([2H])c([2H])c(-c3cccc4ccccc34)c([2H])c2[2H])c([2H])c([2H])c1-c1cccc2ccccc12]

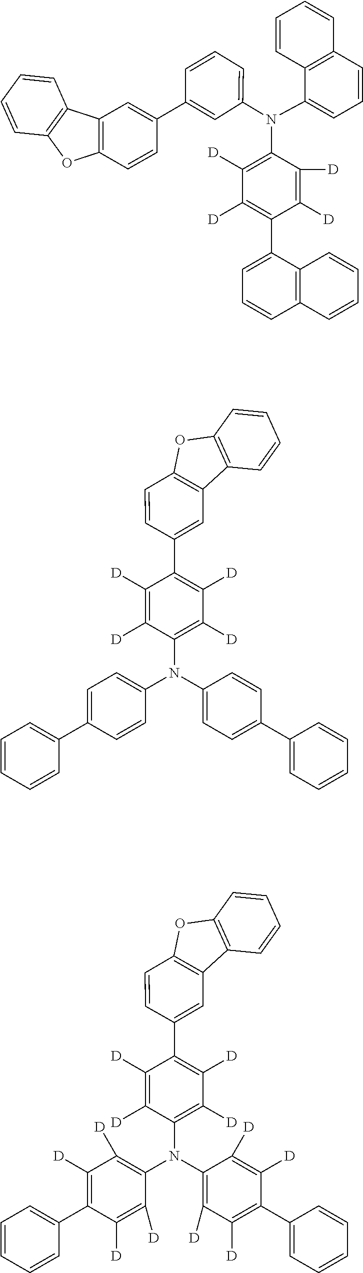 [SMILES: [2H]c1c([2H])c(N(c2cccc(-c3ccc4oc5ccccc5c4c3)c2)c2cccc3ccccc23)c([2H])c([2H])c1-c1cccc2ccccc12 ; [2H]c1c([2H])c(N(c2ccc(-c3ccccc3)cc2)c2ccc(-c3ccccc3)cc2)c([2H])c([2H])c1-c1ccc2oc3ccccc3c2c1 ; [2H]c1c([2H])c(N(c2c([2H])c([2H])c(-c3ccccc3)c([2H])c2[2H])c2c([2H])c([2H])c(-c3ccc4oc5ccccc5c4c3)c([2H])c2[2H])c([2H])c([2H])c1-c1ccccc1]

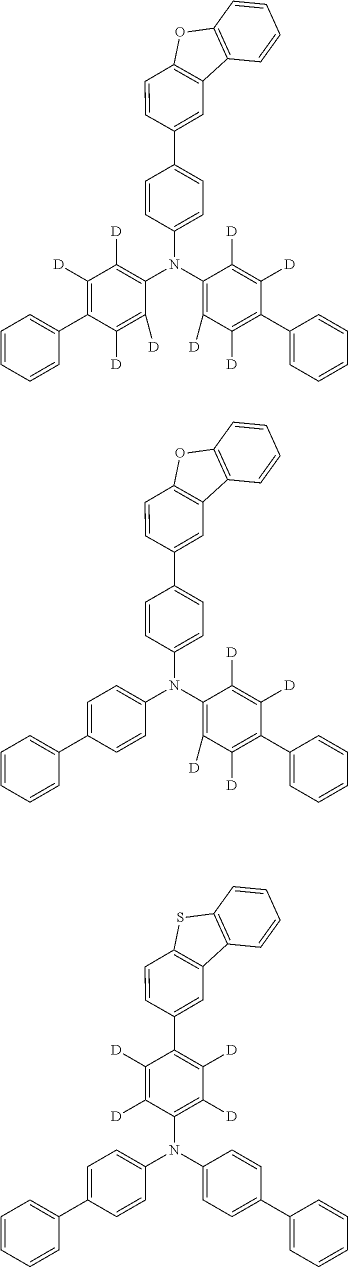 [SMILES: [2H]c1c([2H])c(N(c2ccc(-c3ccc4oc5ccccc5c4c3)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4oc5ccccc5c4c3)cc2)c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1ccc2sc3ccccc3c2c1]

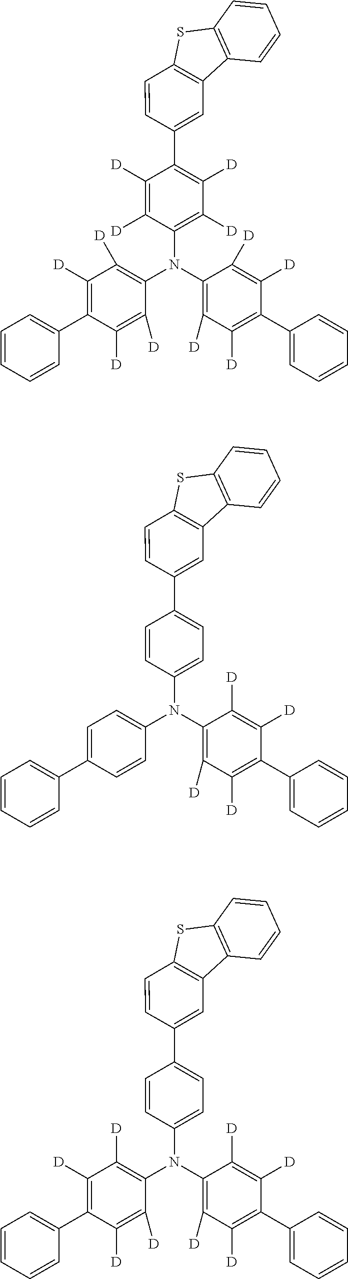 [SMILES: [2H]c1c([2H])c(N(c2c([2H])c([2H])c(-c3ccccc3)c([2H])c2[2H])c2c([2H])c([2H])c(-c3ccc4sc5ccccc5c4c3)c([2H])c2[2H])c([2H])c([2H])c1-c1ccccc1 ; [2H]c1c([2H])c(N(c2ccc(-c3ccccc3)cc2)c2ccc(-c3ccc4sc5ccccc5c4c3)cc2)c([2H])c([2H])c1-c1ccccc1 ; [2H]c1c([2H])c(N(c2ccc(-c3ccc4sc5ccccc5c4c3)cc2)c2c([2H])c([2H])c(-c3ccccc3)c([2H])c2[2H])c([2H])c([2H])c1-c1ccccc1]

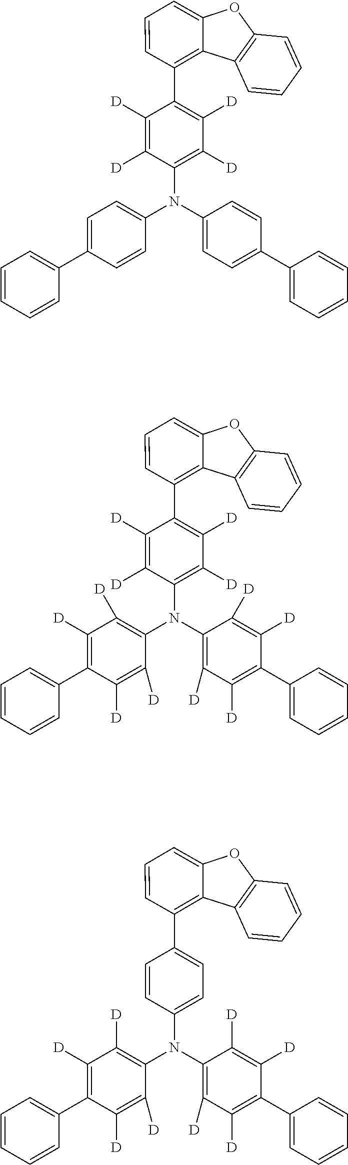 [SMILES: [2H]c1c([2H])c(N(c2ccc(-c3ccccc3)cc2)c2ccc(-c3ccccc3)cc2)c([2H])c([2H])c1-c1cccc2oc3ccccc3c12 ; [2H]c1c([2H])c(N(c2c([2H])c([2H])c(-c3ccccc3)c([2H])c2[2H])c2c([2H])c([2H])c(-c3cccc4oc5ccccc5c34)c([2H])c2[2H])c([2H])c([2H])c1-c1ccccc1 ; [2H]c1c([2H])c(N(c2ccc(-c3cccc4oc5ccccc5c34)cc2)c2c([2H])c([2H])c(-c3ccccc3)c([2H])c2[2H])c([2H])c([2H])c1-c1ccccc1]

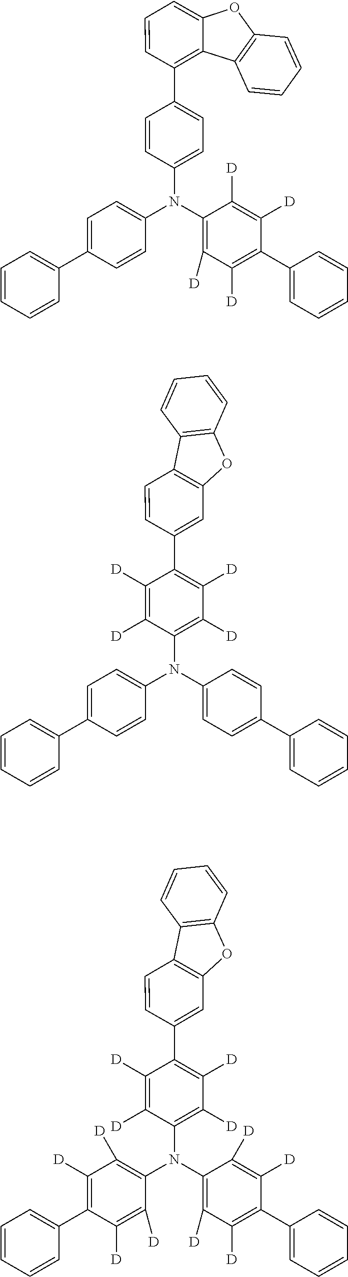 [SMILES: [2H]c1c([2H])c(N(c2ccc(-c3ccccc3)cc2)c2ccc(-c3cccc4oc5ccccc5c34)cc2)c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1ccc2c(c1)oc1ccccc12 ; [2H]c1c([2H])c(N(c2c([2H])c([2H])c(-c3ccccc3)c([2H])c2[2H])c2c([2H])c([2H])c(-c3ccc4c(c3)oc3ccccc34)c([2H])c2[2H])c([2H])c([2H])c1-c1ccccc1]

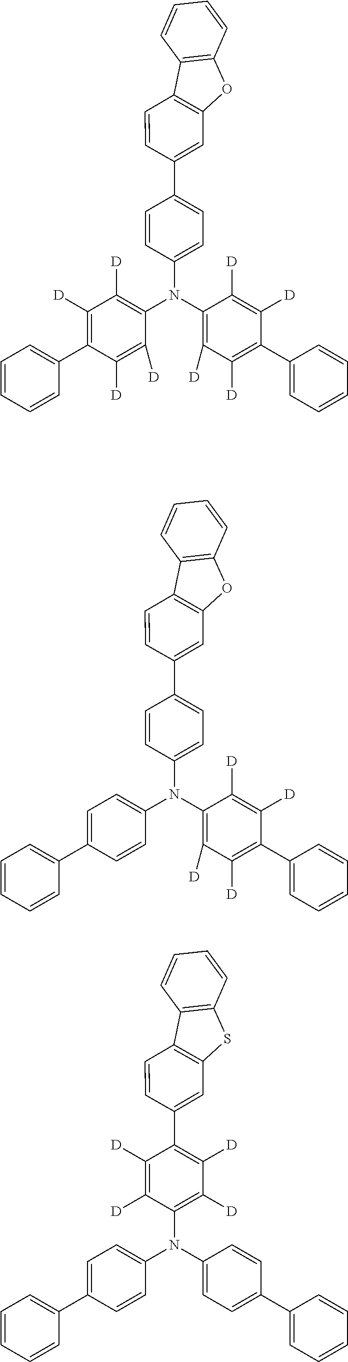 [SMILES: [2H]c1c([2H])c(N(c2ccc(-c3ccc4c(c3)oc3ccccc34)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4c(c3)oc3ccccc34)cc2)c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1ccc2c(c1)sc1ccccc12]

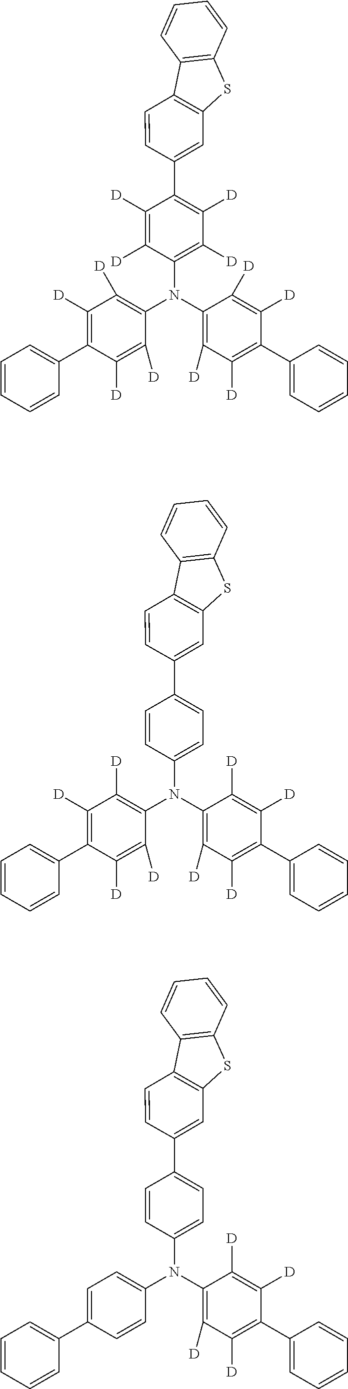 [SMILES: [2H]c1c([2H])c(N(c2c([2H])c([2H])c(-c3ccccc3)c([2H])c2[2H])c2c([2H])c([2H])c(-c3ccc4c(c3)sc3ccccc34)c([2H])c2[2H])c([2H])c([2H])c1-c1ccccc1 ; [2H]c1c([2H])c(N(c2ccc(-c3ccc4c(c3)sc3ccccc34)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4c(c3)sc3ccccc34)cc2)c([2H])c([2H])c1-c1ccccc1]

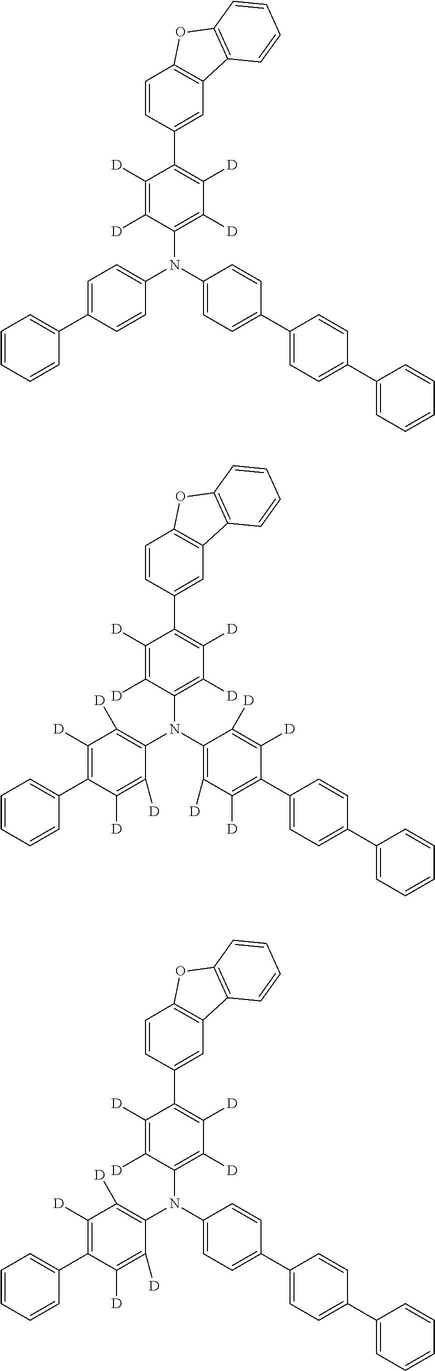 [SMILES: [2H]c1c([2H])c(N(c2ccc(-c3ccccc3)cc2)c2ccc(-c3ccc(-c4ccccc4)cc3)cc2)c([2H])c([2H])c1-c1ccc2oc3ccccc3c2c1 ; [2H]c1c([2H])c(N(c2c([2H])c([2H])c(-c3ccc(-c4ccccc4)cc3)c([2H])c2[2H])c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccc(-c4ccccc4)cc3)cc2)c2c([2H])c([2H])c(-c3ccc4oc5ccccc5c4c3)c([2H])c2[2H])c([2H])c([2H])c1-c1ccccc1]

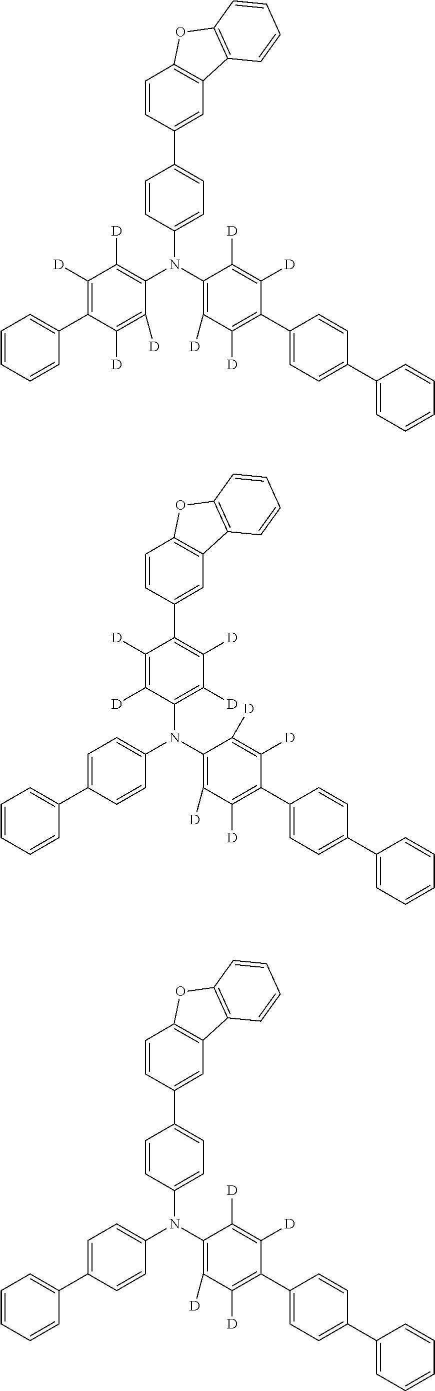 [SMILES: [2H]c1c([2H])c(N(c2ccc(-c3ccc4oc5ccccc5c4c3)cc2)c2c([2H])c([2H])c(-c3ccc(-c4ccccc4)cc3)c([2H])c2[2H])c([2H])c([2H])c1-c1ccccc1 ; [2H]c1c([2H])c(N(c2ccc(-c3ccccc3)cc2)c2c([2H])c([2H])c(-c3ccc4oc5ccccc5c4c3)c([2H])c2[2H])c([2H])c([2H])c1-c1ccc(-c2ccccc2)cc1 ; [2H]c1c([2H])c(N(c2ccc(-c3ccccc3)cc2)c2ccc(-c3ccc4oc5ccccc5c4c3)cc2)c([2H])c([2H])c1-c1ccc(-c2ccccc2)cc1]

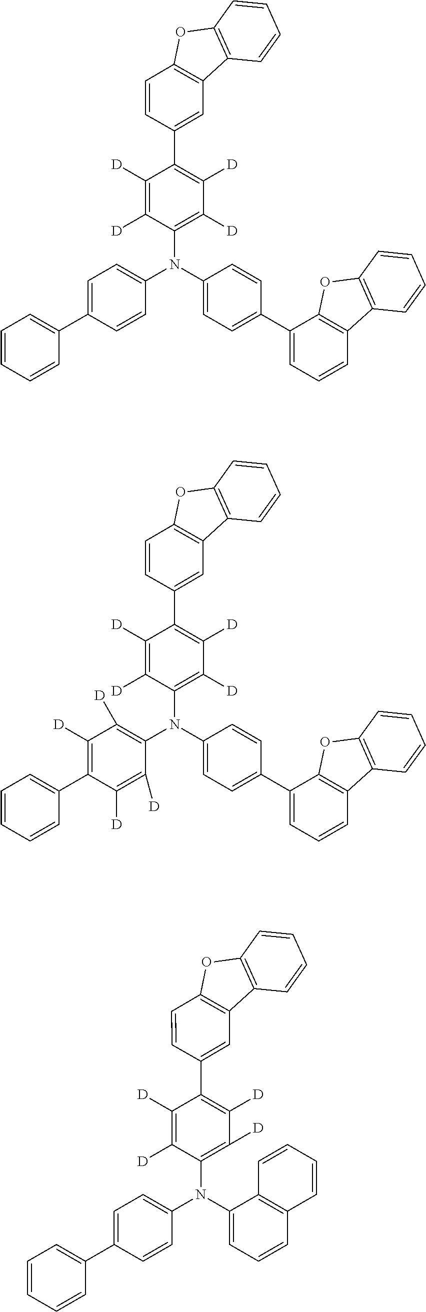 [SMILES: [2H]c1c([2H])c(N(c2ccc(-c3ccccc3)cc2)c2ccc(-c3cccc4c3oc3ccccc34)cc2)c([2H])c([2H])c1-c1ccc2oc3ccccc3c2c1 ; [2H]c1c([2H])c(N(c2ccc(-c3cccc4c3oc3ccccc34)cc2)c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccccc3)cc2)c2cccc3ccccc23)c([2H])c([2H])c1-c1ccc2oc3ccccc3c2c1]

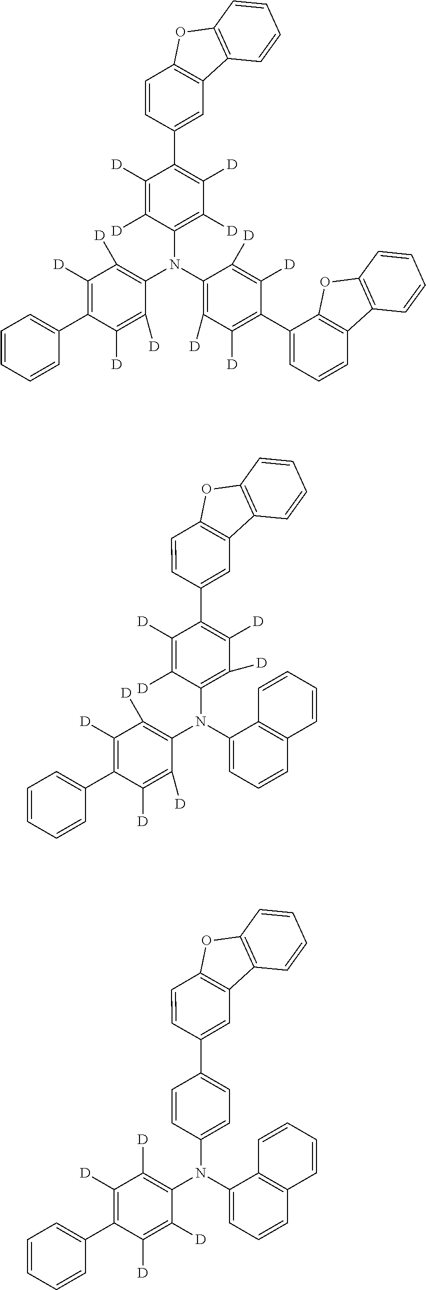 [SMILES: [2H]c1c([2H])c(N(c2c([2H])c([2H])c(-c3ccc4oc5ccccc5c4c3)c([2H])c2[2H])c2c([2H])c([2H])c(-c3cccc4c3oc3ccccc34)c([2H])c2[2H])c([2H])c([2H])c1-c1ccccc1 ; [2H]c1c([2H])c(N(c2c([2H])c([2H])c(-c3ccc4oc5ccccc5c4c3)c([2H])c2[2H])c2cccc3ccccc23)c([2H])c([2H])c1-c1ccccc1 ; [2H]c1c([2H])c(N(c2ccc(-c3ccc4oc5ccccc5c4c3)cc2)c2cccc3ccccc23)c([2H])c([2H])c1-c1ccccc1]

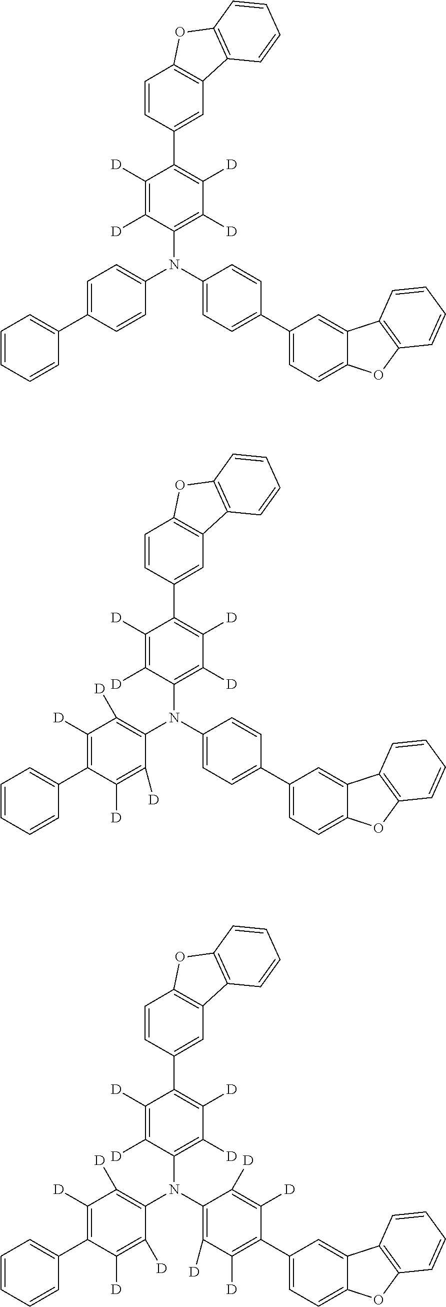 [SMILES: [2H]c1c([2H])c(N(c2ccc(-c3ccccc3)cc2)c2ccc(-c3ccc4oc5ccccc5c4c3)cc2)c([2H])c([2H])c1-c1ccc2oc3ccccc3c2c1 ; [2H]c1c([2H])c(N(c2ccc(-c3ccc4oc5ccccc5c4c3)cc2)c2c([2H])c([2H])c(-c3ccc4oc5ccccc5c4c3)c([2H])c2[2H])c([2H])c([2H])c1-c1ccccc1 ; [2H]c1c([2H])c(N(c2c([2H])c([2H])c(-c3ccc4oc5ccccc5c4c3)c([2H])c2[2H])c2c([2H])c([2H])c(-c3ccc4oc5ccccc5c4c3)c([2H])c2[2H])c([2H])c([2H])c1-c1ccccc1]

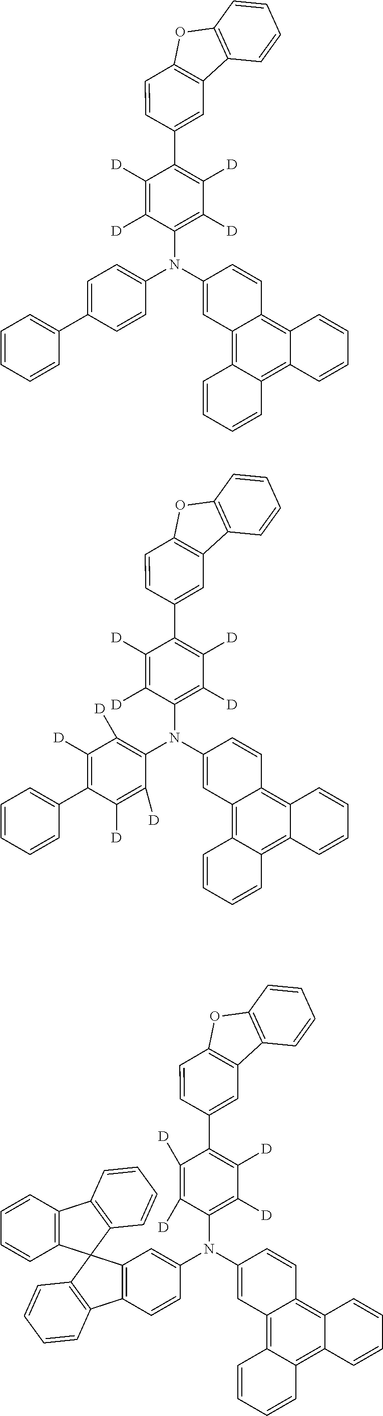

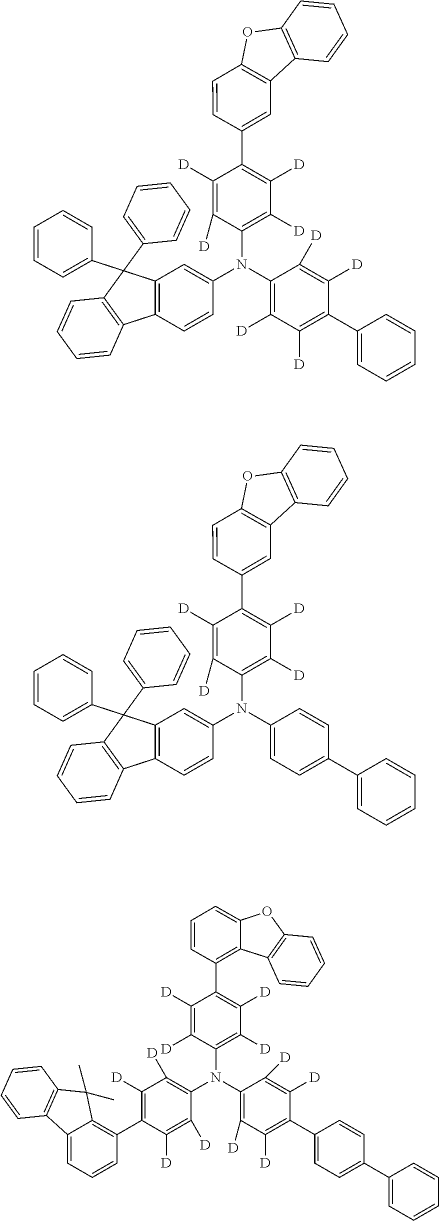 [SMILES: [2H]c1c([2H])c(N(c2ccc3c(c2)C(c2ccccc2)(c2ccccc2)c2ccccc2-3)c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccccc3)cc2)c2ccc3c(c2)C(c2ccccc2)(c2ccccc2)c2ccccc2-3)c([2H])c([2H])c1-c1ccc2oc3ccccc3c2c1 ; [2H]c1c([2H])c(N(c2c([2H])c([2H])c(-c3cccc4c3C(C)(C)c3ccccc3-4)c([2H])c2[2H])c2c([2H])c([2H])c(-c3cccc4oc5ccccc5c34)c([2H])c2[2H])c([2H])c([2H])c1-c1ccc(-c2ccccc2)cc1]

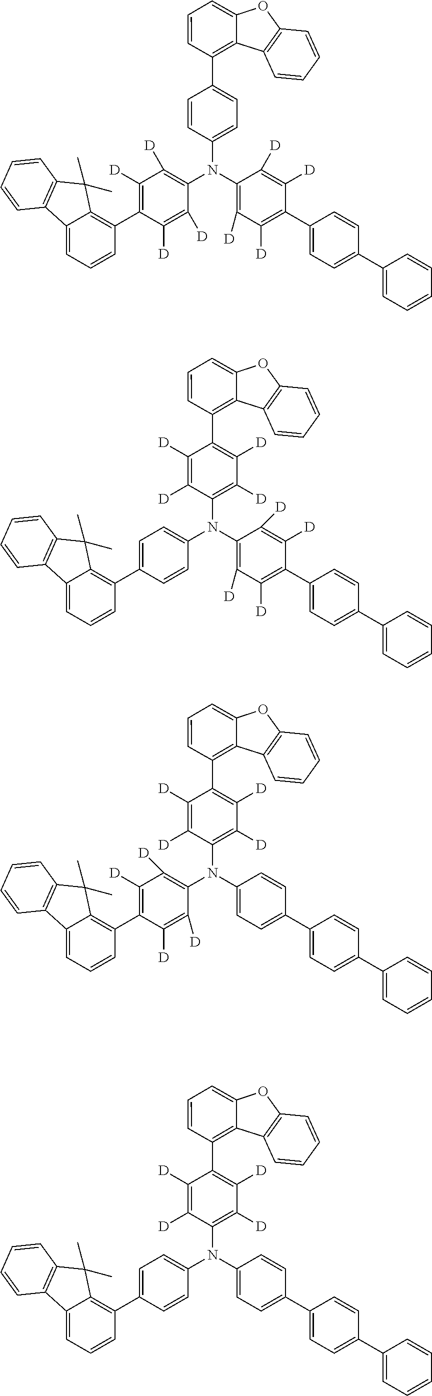

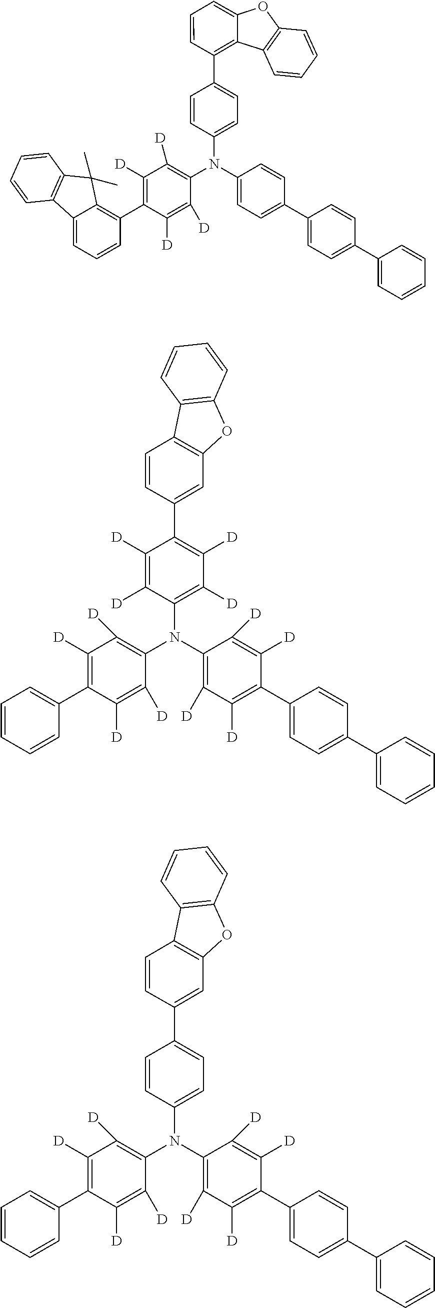 [SMILES: [2H]c1c([2H])c(N(c2ccc(-c3ccc(-c4ccccc4)cc3)cc2)c2ccc(-c3cccc4oc5ccccc5c34)cc2)c([2H])c([2H])c1-c1cccc2c1C(C)(C)c1ccccc1-2 ; [2H]c1c([2H])c(N(c2c([2H])c([2H])c(-c3ccc(-c4ccccc4)cc3)c([2H])c2[2H])c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccc4c(c3)oc3ccccc34)cc2)c2c([2H])c([2H])c(-c3ccc(-c4ccccc4)cc3)c([2H])c2[2H])c([2H])c([2H])c1-c1ccccc1]

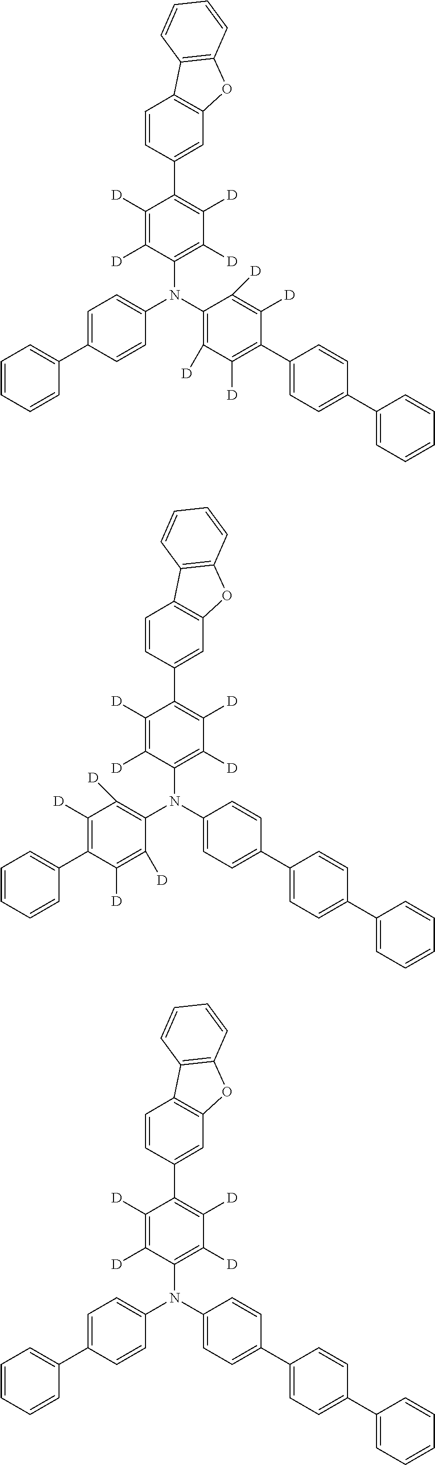 [SMILES: [2H]c1c([2H])c(N(c2ccc(-c3ccccc3)cc2)c2c([2H])c([2H])c(-c3ccc4c(c3)oc3ccccc34)c([2H])c2[2H])c([2H])c([2H])c1-c1ccc(-c2ccccc2)cc1 ; [2H]c1c([2H])c(N(c2ccc(-c3ccc(-c4ccccc4)cc3)cc2)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccccc3)cc2)c2ccc(-c3ccc(-c4ccccc4)cc3)cc2)c([2H])c([2H])c1-c1ccc2c(c1)oc1ccccc12]

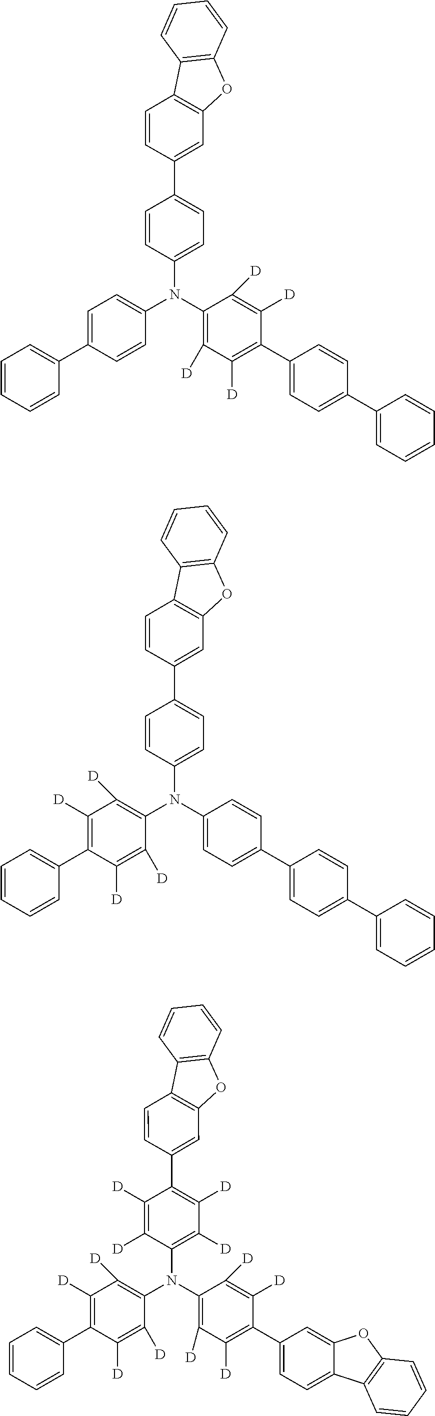 [SMILES: [2H]c1c([2H])c(N(c2ccc(-c3ccccc3)cc2)c2ccc(-c3ccc4c(c3)oc3ccccc34)cc2)c([2H])c([2H])c1-c1ccc(-c2ccccc2)cc1 ; [2H]c1c([2H])c(N(c2ccc(-c3ccc(-c4ccccc4)cc3)cc2)c2ccc(-c3ccc4c(c3)oc3ccccc34)cc2)c([2H])c([2H])c1-c1ccccc1 ; [2H]c1c([2H])c(N(c2c([2H])c([2H])c(-c3ccc4c(c3)oc3ccccc34)c([2H])c2[2H])c2c([2H])c([2H])c(-c3ccc4c(c3)oc3ccccc34)c([2H])c2[2H])c([2H])c([2H])c1-c1ccccc1]

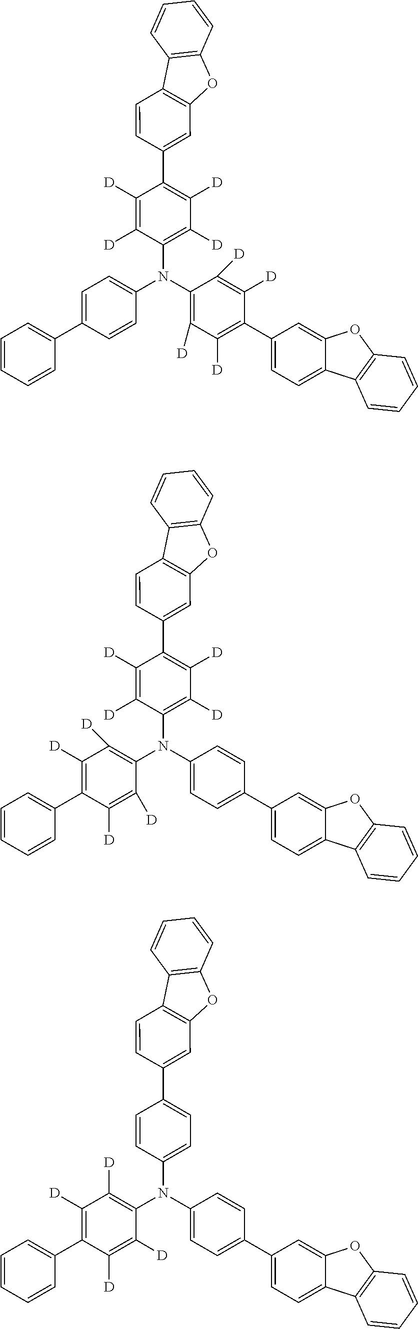 [SMILES: [2H]c1c([2H])c(N(c2ccc(-c3ccccc3)cc2)c2c([2H])c([2H])c(-c3ccc4c(c3)oc3ccccc34)c([2H])c2[2H])c([2H])c([2H])c1-c1ccc2c(c1)oc1ccccc12 ; [2H]c1c([2H])c(N(c2ccc(-c3ccc4c(c3)oc3ccccc34)cc2)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccc4c(c3)oc3ccccc34)cc2)c2ccc(-c3ccc4c(c3)oc3ccccc34)cc2)c([2H])c([2H])c1-c1ccccc1]

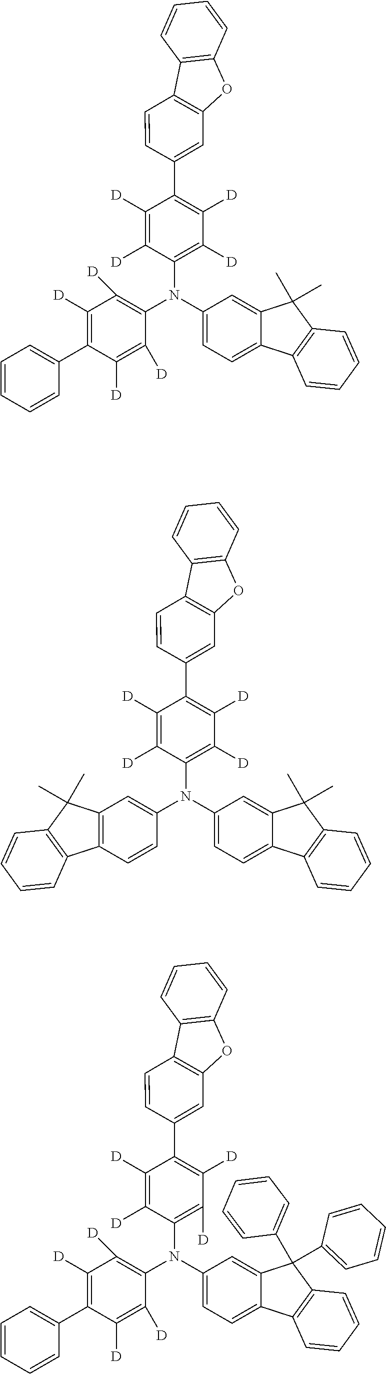 [SMILES: [2H]c1c([2H])c(N(c2ccc3c(c2)C(C)(C)c2ccccc2-3)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc3c(c2)C(C)(C)c2ccccc2-3)c2ccc3c(c2)C(C)(C)c2ccccc2-3)c([2H])c([2H])c1-c1ccc2c(c1)oc1ccccc12 ; [2H]c1c([2H])c(N(c2ccc3c(c2)C(c2ccccc2)(c2ccccc2)c2ccccc2-3)c2c([2H])c([2H])c(-c3ccc4c(c3)oc3ccccc34)c([2H])c2[2H])c([2H])c([2H])c1-c1ccccc1]

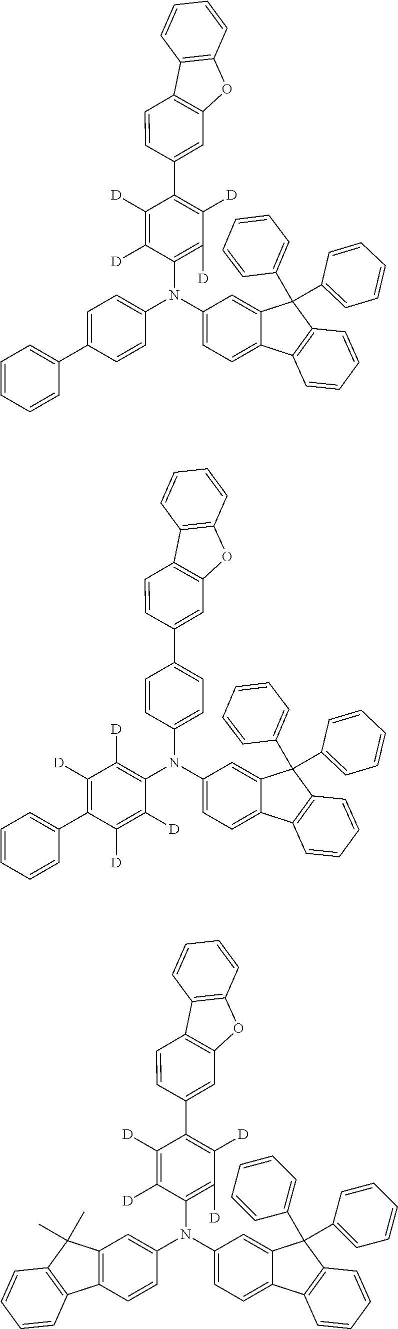 [SMILES: [2H]c1c([2H])c(N(c2ccc(-c3ccccc3)cc2)c2ccc3c(c2)C(c2ccccc2)(c2ccccc2)c2ccccc2-3)c([2H])c([2H])c1-c1ccc2c(c1)oc1ccccc12 ; [2H]c1c([2H])c(N(c2ccc(-c3ccc4c(c3)oc3ccccc34)cc2)c2ccc3c(c2)C(c2ccccc2)(c2ccccc2)c2ccccc2-3)c([2H])c([2H])c1-c1ccccc1 ; [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1ccc2c(c1)oc1ccccc12]

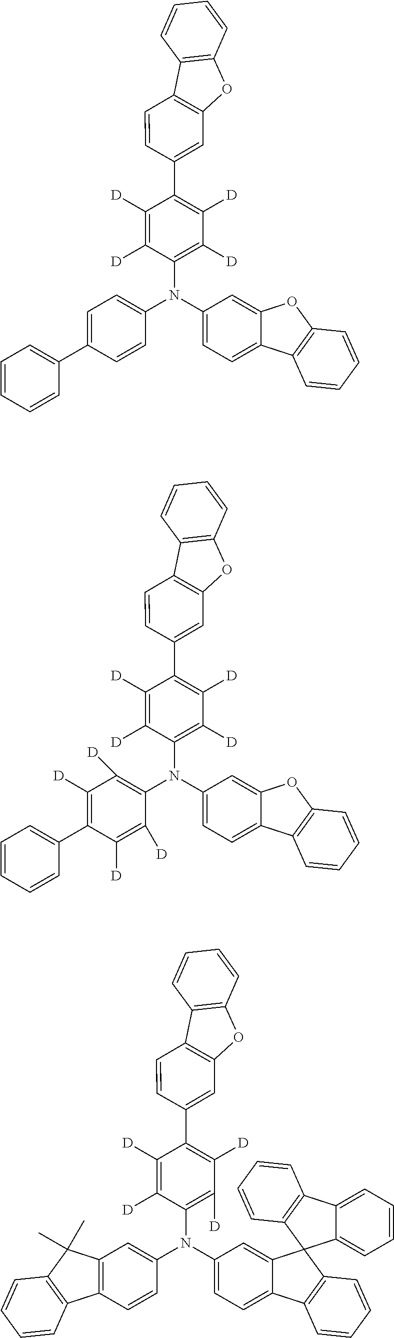 [SMILES: [2H]c1c([2H])c(N(c2ccc(-c3ccccc3)cc2)c2ccc3c(c2)oc2ccccc23)c([2H])c([2H])c1-c1ccc2c(c1)oc1ccccc12 ; [2H]c1c([2H])c(N(c2ccc3c(c2)oc2ccccc23)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc3c(c2)C(C)(C)c2ccccc2-3)c2ccc3c(c2)C2(c4ccccc4-c4ccccc42)c2ccccc2-3)c([2H])c([2H])c1-c1ccc2c(c1)oc1ccccc12]

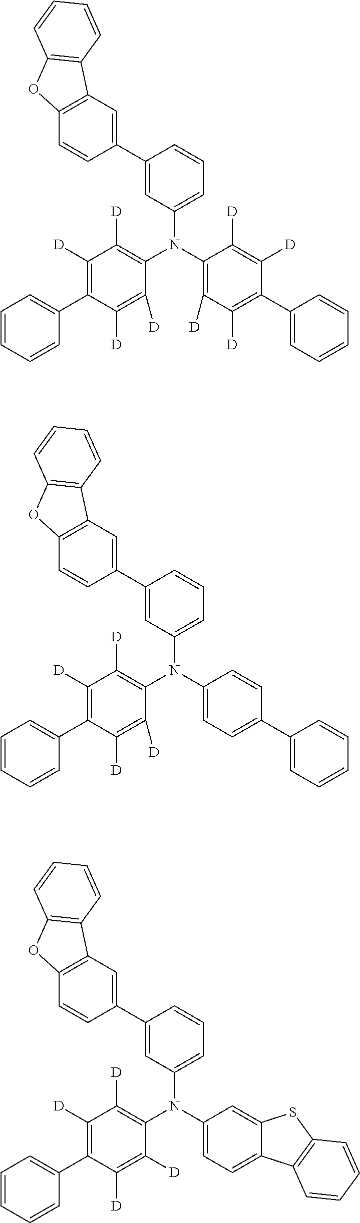 [SMILES: [2H]c1c([2H])c(N(c2cccc(-c3ccc4oc5ccccc5c4c3)c2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2cccc(-c3ccc4oc5ccccc5c4c3)c2)c([2H])c([2H])c1-c1ccccc1 ; [2H]c1c([2H])c(N(c2cccc(-c3ccc4oc5ccccc5c4c3)c2)c2ccc3c(c2)sc2ccccc23)c([2H])c([2H])c1-c1ccccc1]

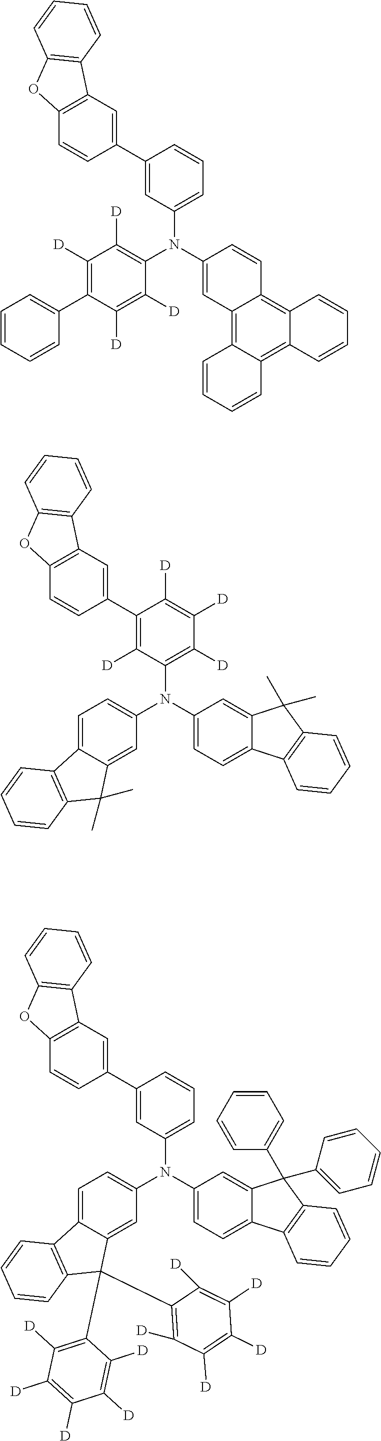 [SMILES: [2H]c1c([2H])c(N(c2cccc(-c3ccc4oc5ccccc5c4c3)c2)c2ccc3c4ccccc4c4ccccc4c3c2)c([2H])c([2H])c1-c1ccccc1 ; [2H]c1c([2H])c(-c2ccc3oc4ccccc4c3c2)c([2H])c(N(c2ccc3c(c2)C(C)(C)c2ccccc2-3)c2ccc3c(c2)C(C)(C)c2ccccc2-3)c1[2H] ; [2H]c1c([2H])c([2H])c(C2(c3c([2H])c([2H])c([2H])c([2H])c3[2H])c3ccccc3-c3ccc(N(c4cccc(-c5ccc6oc7ccccc7c6c5)c4)c4ccc5c(c4)C(c4ccccc4)(c4ccccc4)c4ccccc4-5)cc32)c([2H])c1[2H]]

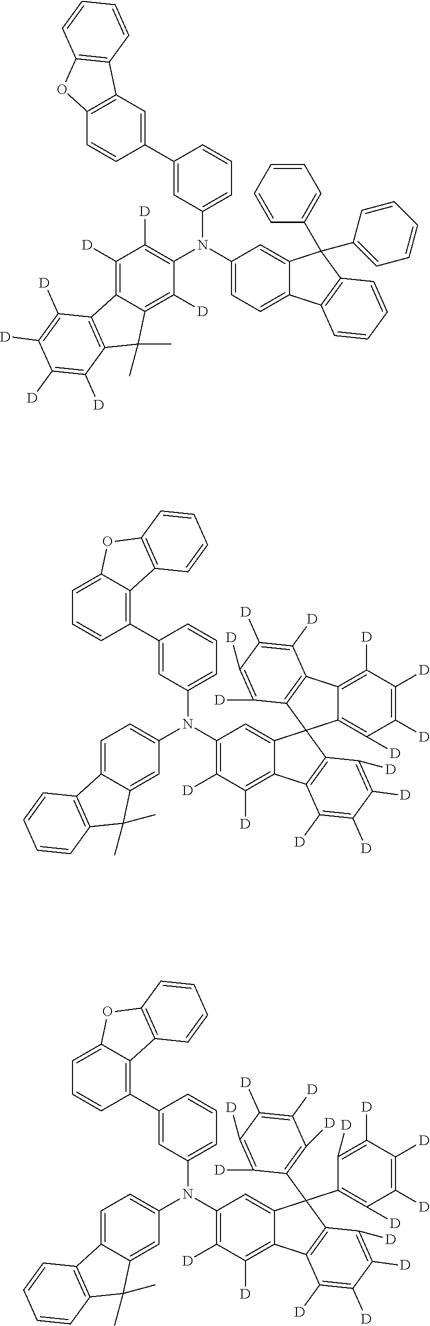

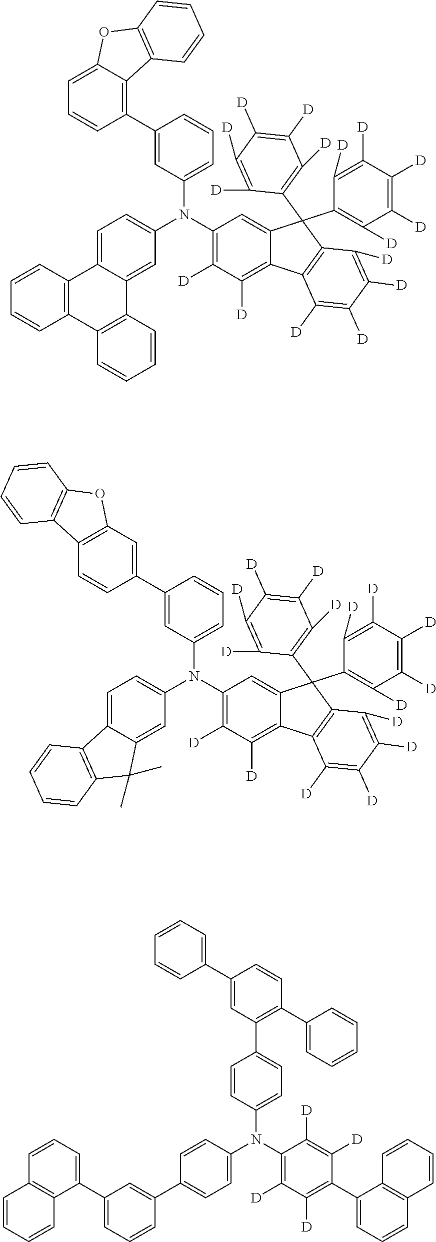

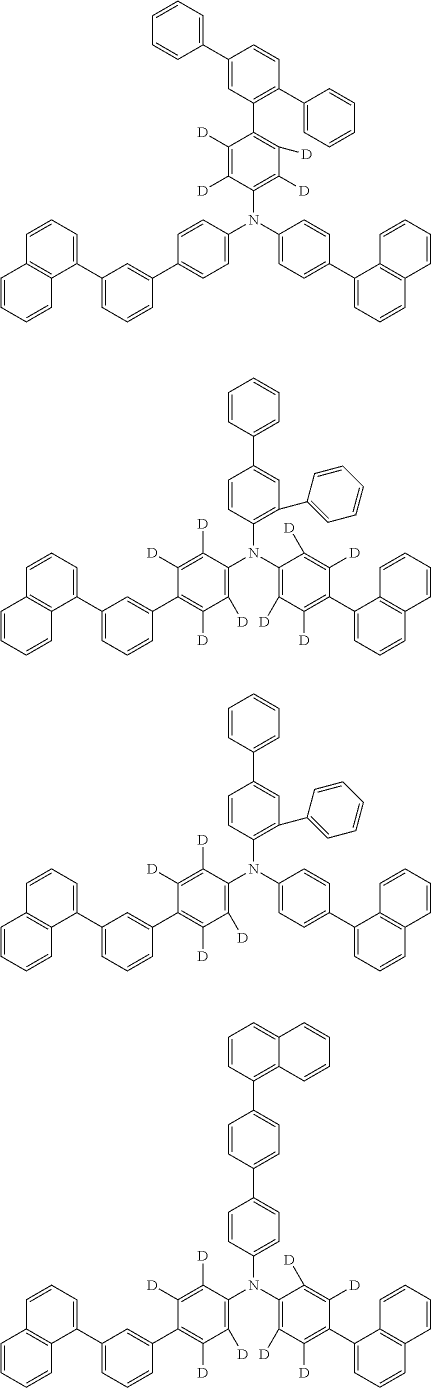

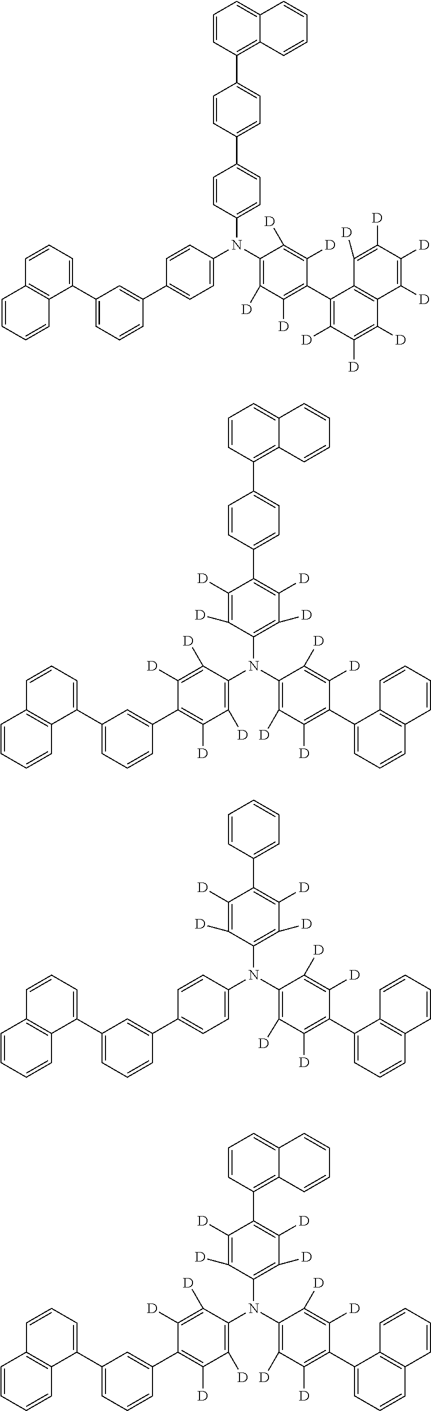

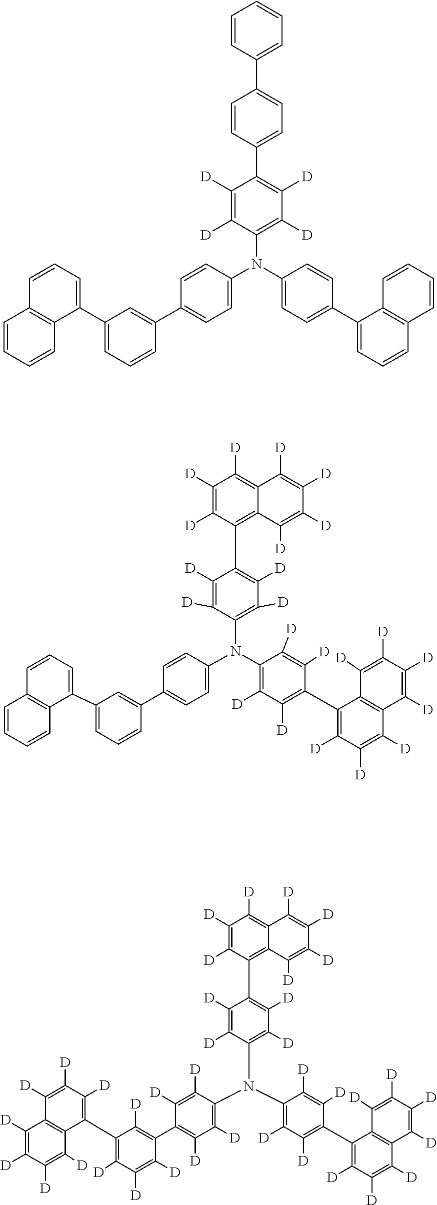

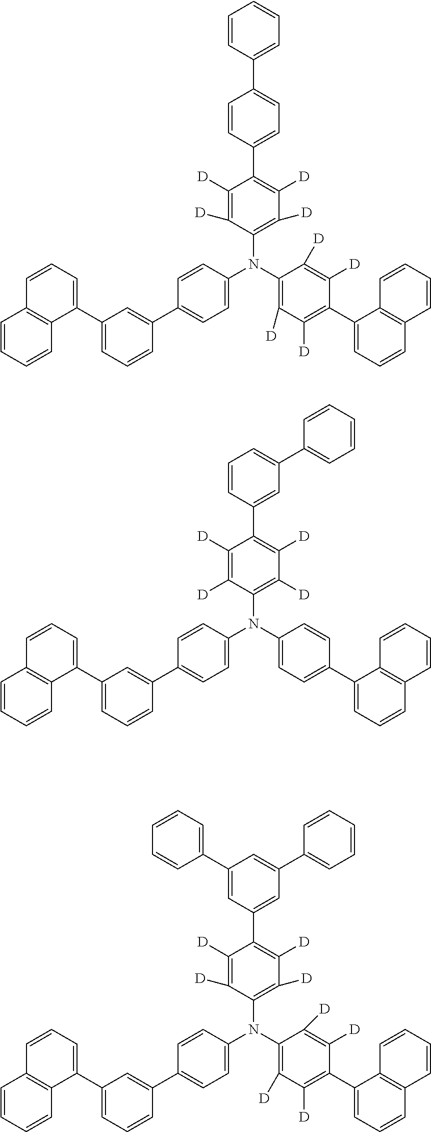 [SMILES: [2H]c1c([2H])c(N(c2ccc(-c3cccc(-c4cccc5ccccc45)c3)cc2)c2c([2H])c([2H])c(-c3cccc4ccccc34)c([2H])c2[2H])c([2H])c([2H])c1-c1ccc(-c2ccccc2)cc1 ; [2H]c1c([2H])c(N(c2ccc(-c3cccc(-c4cccc5ccccc45)c3)cc2)c2ccc(-c3cccc4ccccc34)cc2)c([2H])c([2H])c1-c1cccc(-c2ccccc2)c1 ; [2H]c1c([2H])c(N(c2ccc(-c3cccc(-c4cccc5ccccc45)c3)cc2)c2c([2H])c([2H])c(-c3cccc4ccccc34)c([2H])c2[2H])c([2H])c([2H])c1-c1cc(-c2ccccc2)cc(-c2ccccc2)c1]

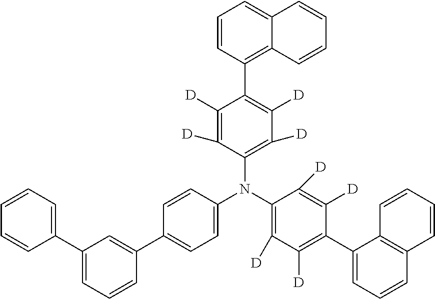 [SMILES: [2H]c1c([2H])c(N(c2ccc(-c3cccc(-c4ccccc4)c3)cc2)c2c([2H])c([2H])c(-c3cccc4ccccc34)c([2H])c2[2H])c([2H])c([2H])c1-c1cccc2ccccc12]

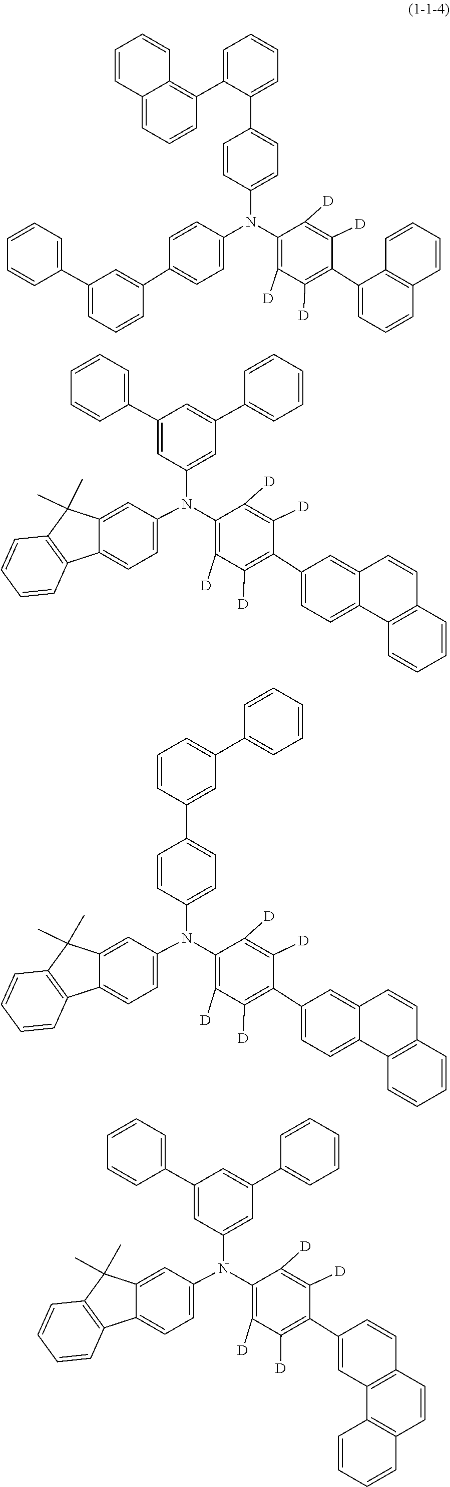

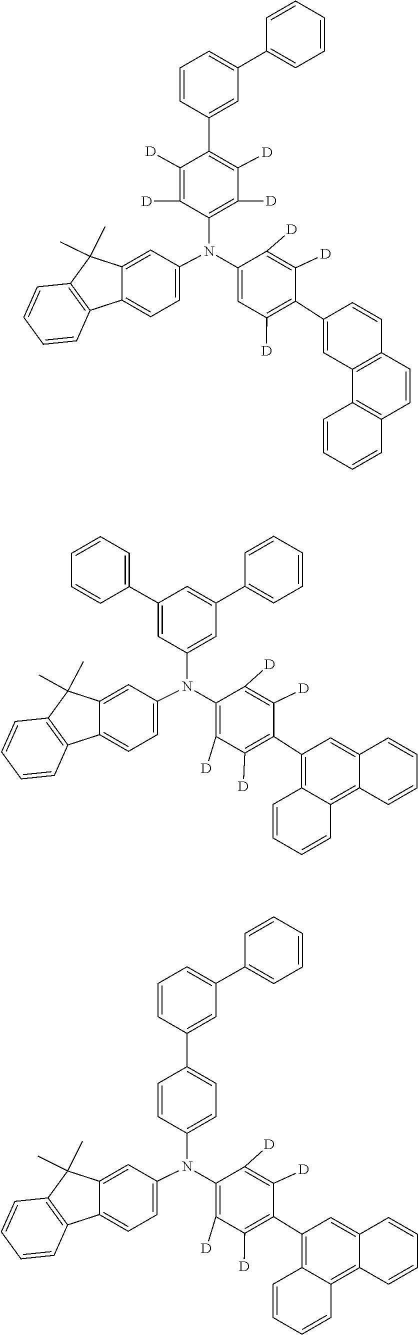 [SMILES: [2H]c1cc(N(c2ccc3c(c2)C(C)(C)c2ccccc2-3)c2c([2H])c([2H])c(-c3cccc(-c4ccccc4)c3)c([2H])c2[2H])c([2H])c([2H])c1-c1ccc2ccc3ccccc3c2c1 ; [2H]c1c([2H])c(N(c2cc(-c3ccccc3)cc(-c3ccccc3)c2)c2ccc3c(c2)C(C)(C)c2ccccc2-3)c([2H])c([2H])c1-c1cc2ccccc2c2ccccc12 ; [2H]c1c([2H])c(N(c2ccc(-c3cccc(-c4ccccc4)c3)cc2)c2ccc3c(c2)C(C)(C)c2ccccc2-3)c([2H])c([2H])c1-c1cc2ccccc2c2ccccc12]

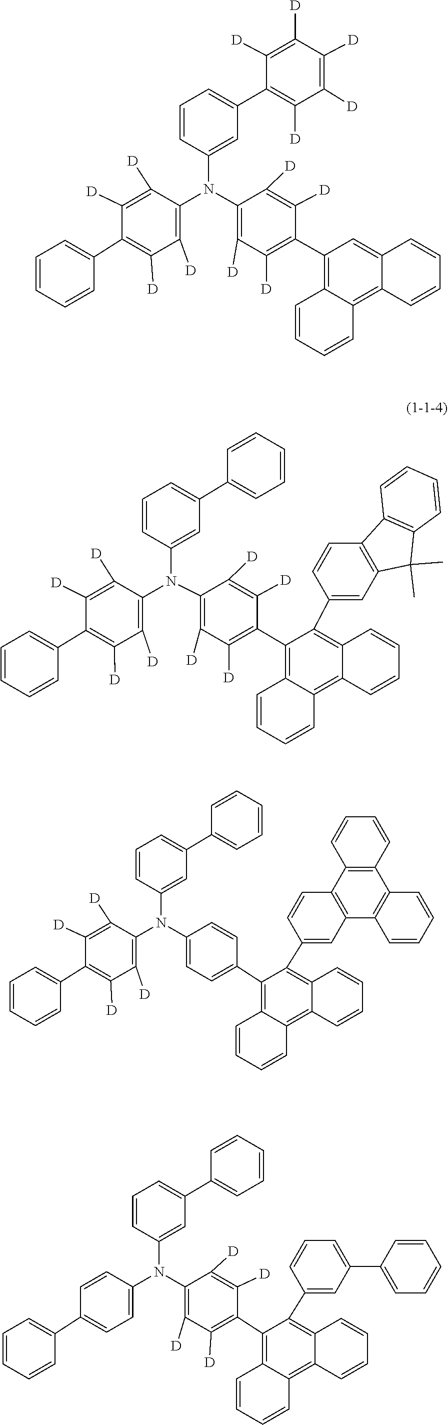

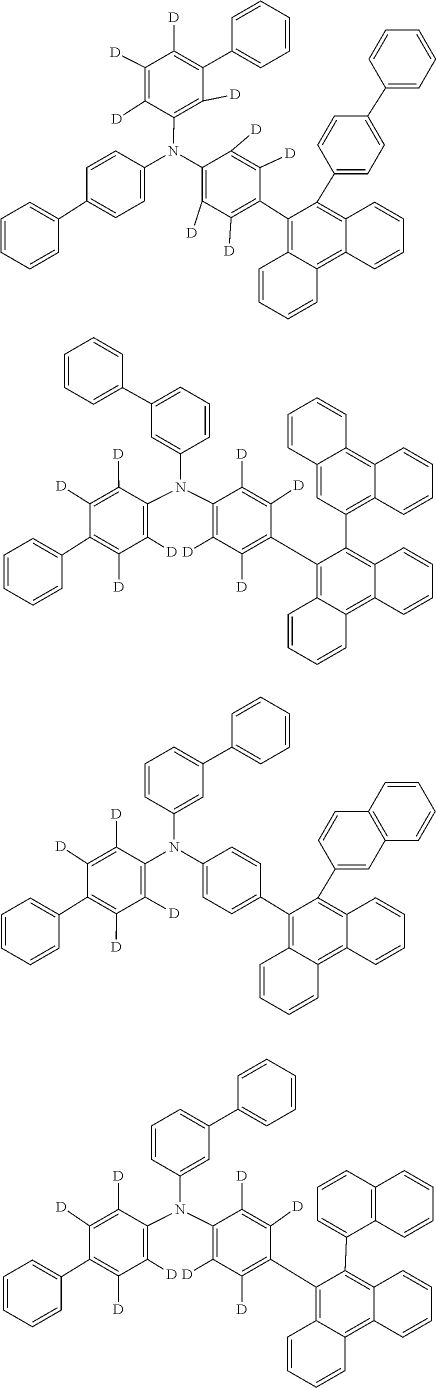

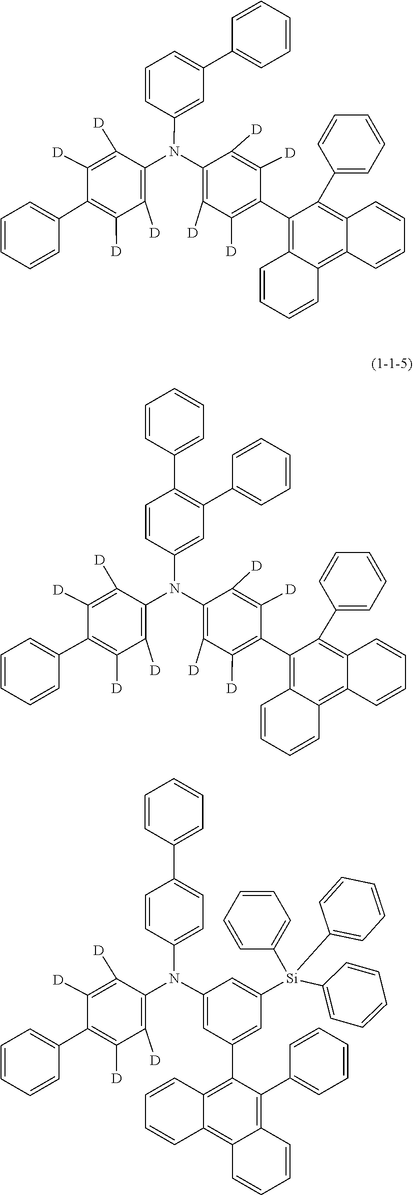 [SMILES: [2H]c1c([2H])c(N(c2cccc(-c3ccccc3)c2)c2c([2H])c([2H])c(-c3c(-c4ccccc4)c4ccccc4c4ccccc34)c([2H])c2[2H])c([2H])c([2H])c1-c1ccccc1 ; [2H]c1c([2H])c(N(c2ccc(-c3ccccc3)c(-c3ccccc3)c2)c2c([2H])c([2H])c(-c3c(-c4ccccc4)c4ccccc4c4ccccc34)c([2H])c2[2H])c([2H])c([2H])c1-c1ccccc1 ; [2H]c1c([2H])c(N(c2ccc(-c3ccccc3)cc2)c2cc(-c3c(-c4ccccc4)c4ccccc4c4ccccc34)cc([Si](c3ccccc3)(c3ccccc3)c3ccccc3)c2)c([2H])c([2H])c1-c1ccccc1]

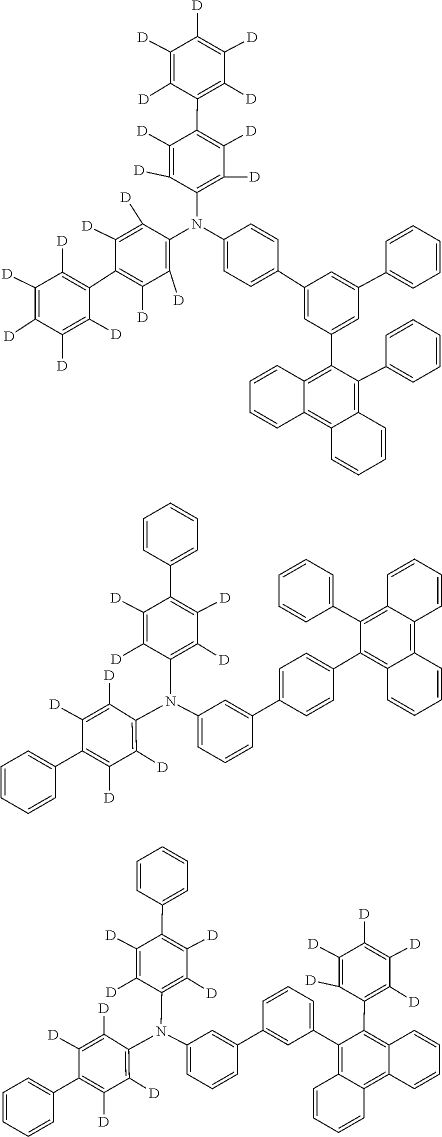

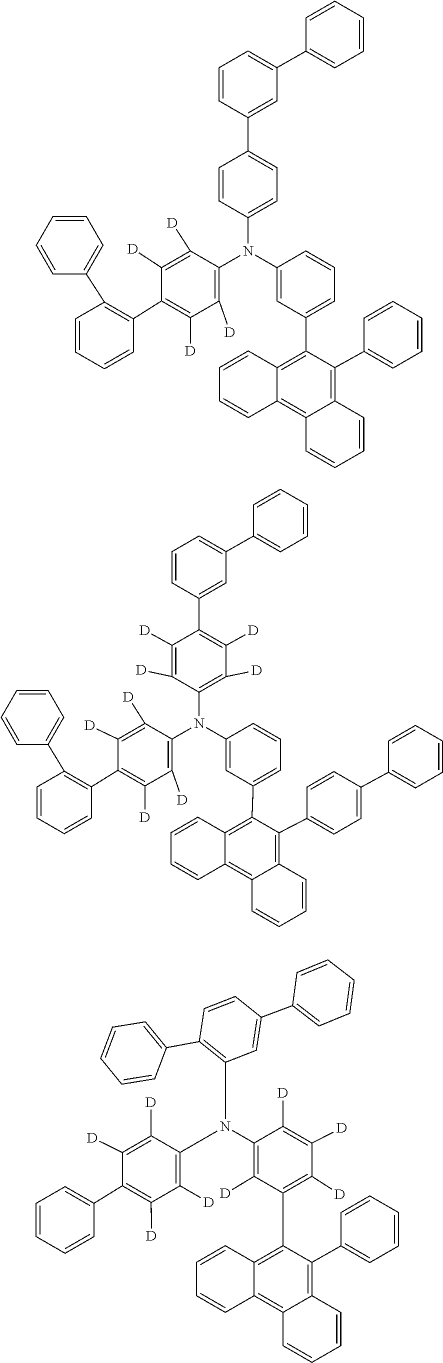

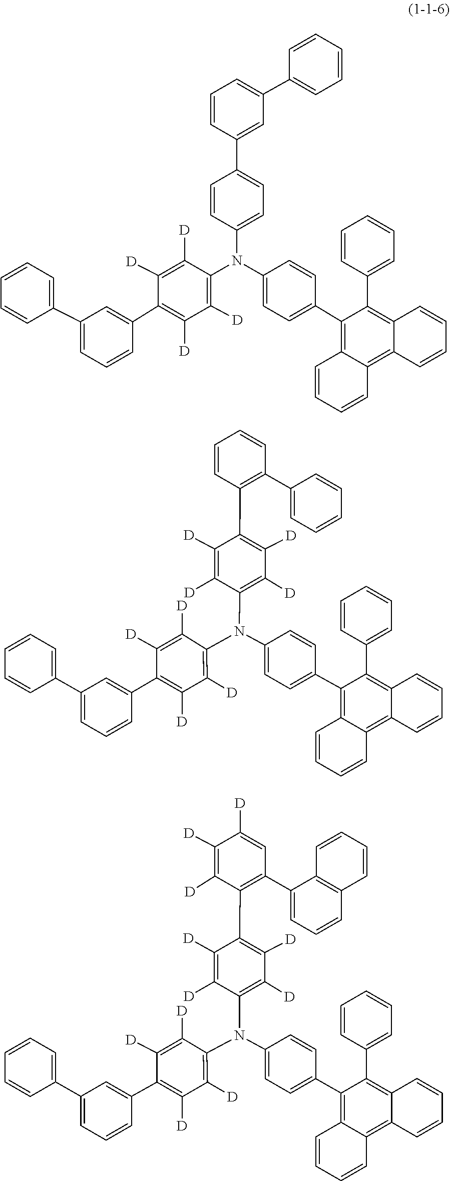 [SMILES: [2H]c1c([2H])c(N(c2ccc(-c3cccc(-c4ccccc4)c3)cc2)c2ccc(-c3c(-c4ccccc4)c4ccccc4c4ccccc34)cc2)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2c([2H])c([2H])c(-c3ccccc3-c3ccccc3)c([2H])c2[2H])c([2H])c([2H])c1-c1cccc(-c2ccccc2)c1 ; [2H]c1cc(-c2cccc3ccccc23)c(-c2c([2H])c([2H])c(N(c3ccc(-c4c(-c5ccccc5)c5ccccc5c5ccccc45)cc3)c3c([2H])c([2H])c(-c4cccc(-c5ccccc5)c4)c([2H])c3[2H])c([2H])c2[2H])c([2H])c1[2H]]

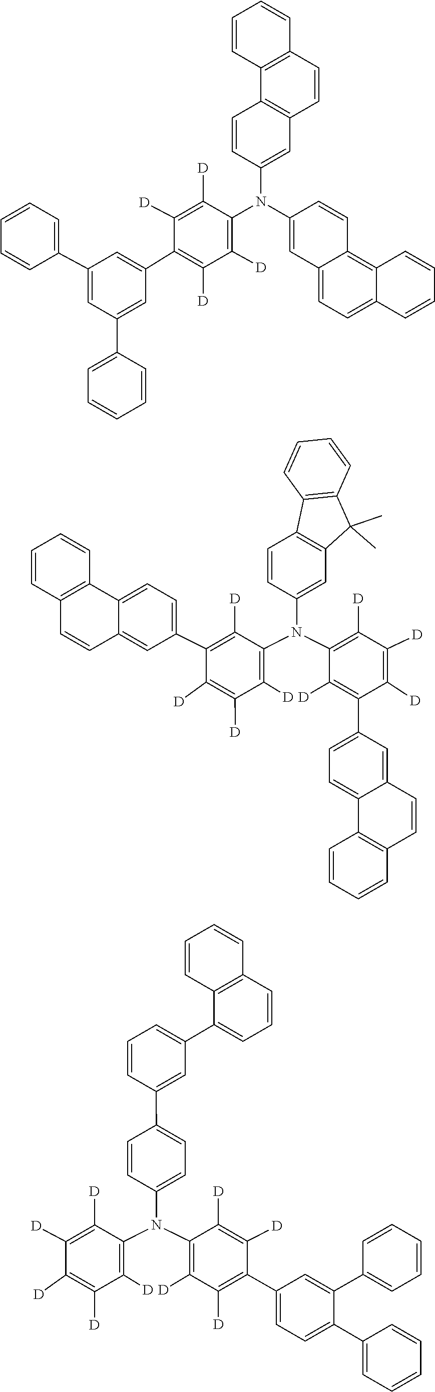 [SMILES: [2H]c1c([2H])c(N(c2ccc3c(ccc4ccccc43)c2)c2ccc3c(ccc4ccccc43)c2)c([2H])c([2H])c1-c1cc(-c2ccccc2)cc(-c2ccccc2)c1 ; [2H]c1c([2H])c(-c2ccc3c(ccc4ccccc43)c2)c([2H])c(N(c2ccc3c(c2)C(C)(C)c2ccccc2-3)c2c([2H])c([2H])c([2H])c(-c3ccc4c(ccc5ccccc54)c3)c2[2H])c1[2H] ; [2H]c1c([2H])c([2H])c(N(c2ccc(-c3cccc(-c4cccc5ccccc45)c3)cc2)c2c([2H])c([2H])c(-c3ccc(-c4ccccc4)c(-c4ccccc4)c3)c([2H])c2[2H])c([2H])c1[2H]]

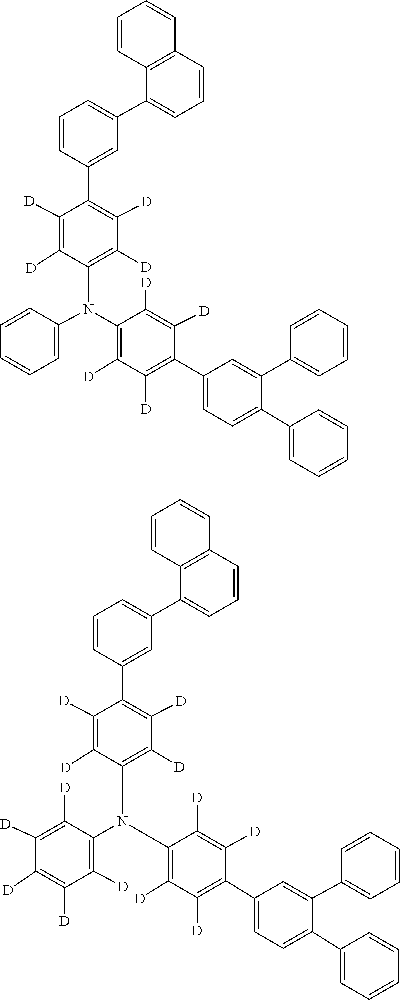 [SMILES: [2H]c1c([2H])c(N(c2ccccc2)c2c([2H])c([2H])c(-c3ccc(-c4ccccc4)c(-c4ccccc4)c3)c([2H])c2[2H])c([2H])c([2H])c1-c1cccc(-c2cccc3ccccc23)c1 ; [2H]c1c([2H])c([2H])c(N(c2c([2H])c([2H])c(-c3cccc(-c4cccc5ccccc45)c3)c([2H])c2[2H])c2c([2H])c([2H])c(-c3ccc(-c4ccccc4)c(-c4ccccc4)c3)c([2H])c2[2H])c([2H])c1[2H]]

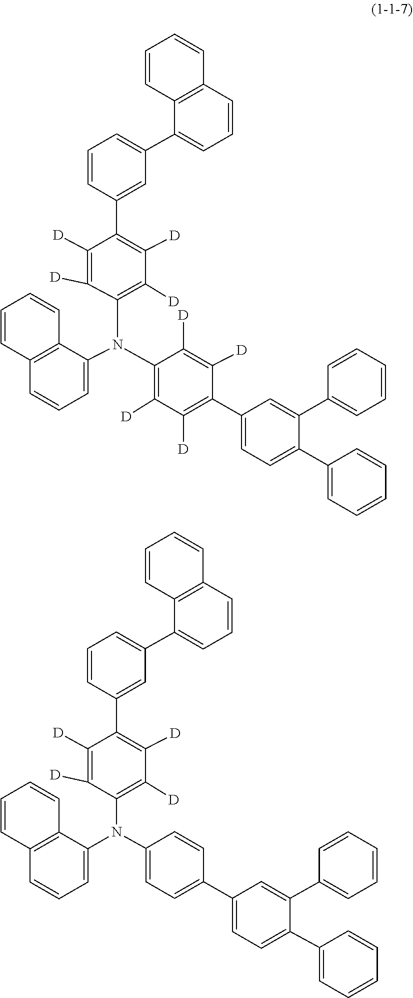 [SMILES: [2H]c1c([2H])c(N(c2c([2H])c([2H])c(-c3ccc(-c4ccccc4)c(-c4ccccc4)c3)c([2H])c2[2H])c2cccc3ccccc23)c([2H])c([2H])c1-c1cccc(-c2cccc3ccccc23)c1 ; [2H]c1c([2H])c(N(c2ccc(-c3ccc(-c4ccccc4)c(-c4ccccc4)c3)cc2)c2cccc3ccccc23)c([2H])c([2H])c1-c1cccc(-c2cccc3ccccc23)c1]

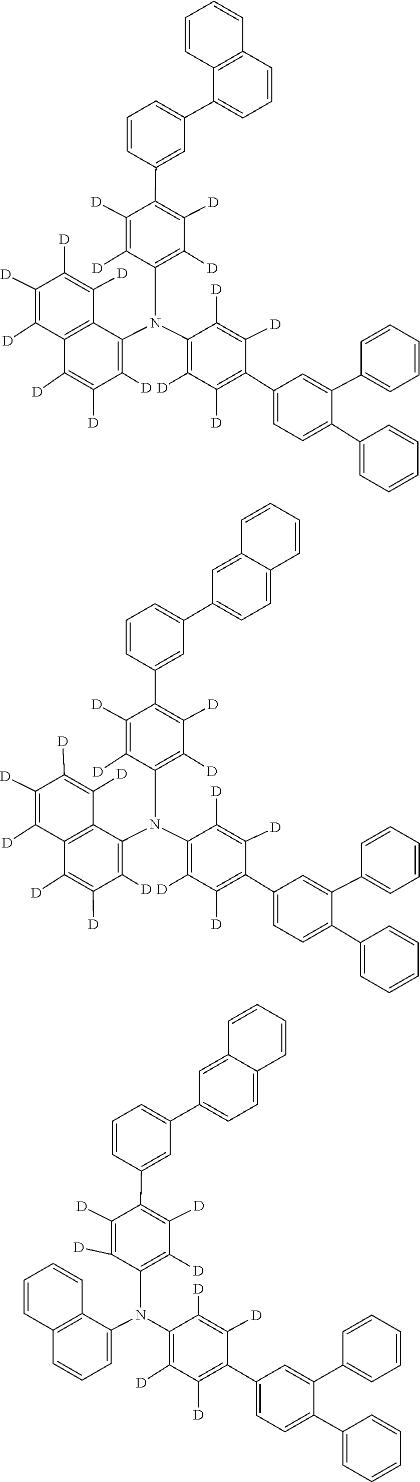

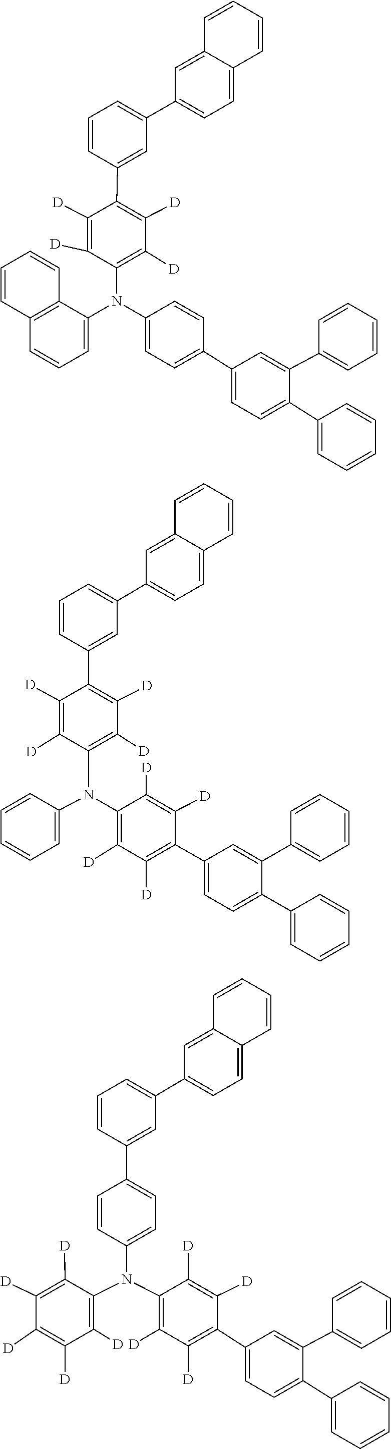 [SMILES: [2H]c1c([2H])c(N(c2ccc(-c3ccc(-c4ccccc4)c(-c4ccccc4)c3)cc2)c2cccc3ccccc23)c([2H])c([2H])c1-c1cccc(-c2ccc3ccccc3c2)c1 ; [2H]c1c([2H])c(N(c2ccccc2)c2c([2H])c([2H])c(-c3ccc(-c4ccccc4)c(-c4ccccc4)c3)c([2H])c2[2H])c([2H])c([2H])c1-c1cccc(-c2ccc3ccccc3c2)c1 ; [2H]c1c([2H])c([2H])c(N(c2ccc(-c3cccc(-c4ccc5ccccc5c4)c3)cc2)c2c([2H])c([2H])c(-c3ccc(-c4ccccc4)c(-c4ccccc4)c3)c([2H])c2[2H])c([2H])c1[2H]]

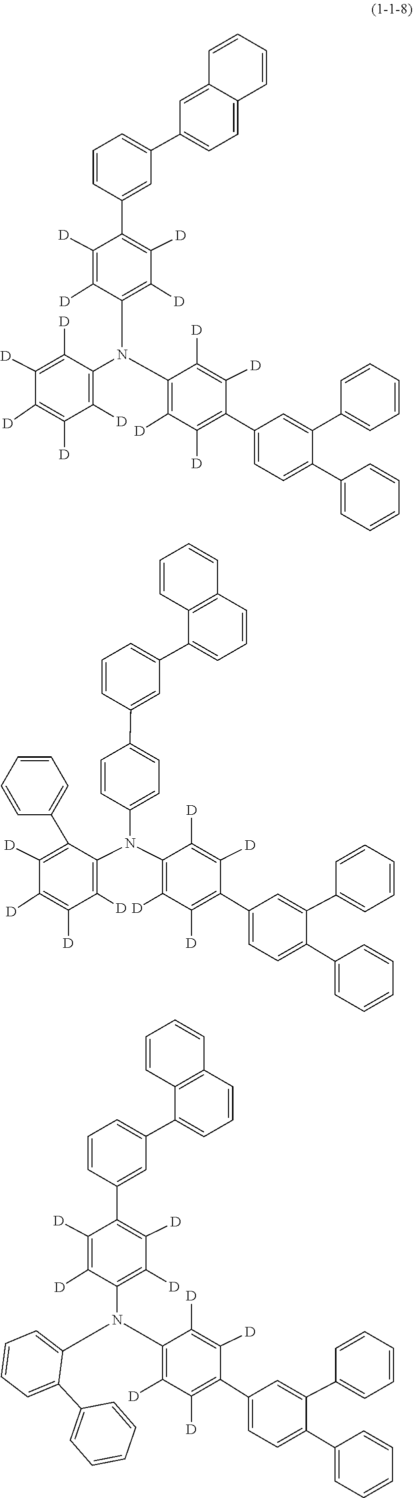 [SMILES: [2H]c1c([2H])c([2H])c(N(c2c([2H])c([2H])c(-c3cccc(-c4ccc5ccccc5c4)c3)c([2H])c2[2H])c2c([2H])c([2H])c(-c3ccc(-c4ccccc4)c(-c4ccccc4)c3)c([2H])c2[2H])c([2H])c1[2H] ; [2H]c1c([2H])c([2H])c(N(c2ccc(-c3cccc(-c4cccc5ccccc45)c3)cc2)c2c([2H])c([2H])c(-c3ccc(-c4ccccc4)c(-c4ccccc4)c3)c([2H])c2[2H])c(-c2ccccc2)c1[2H] ; [2H]c1c([2H])c(N(c2ccccc2-c2ccccc2)c2c([2H])c([2H])c(-c3ccc(-c4ccccc4)c(-c4ccccc4)c3)c([2H])c2[2H])c([2H])c([2H])c1-c1cccc(-c2cccc3ccccc23)c1]

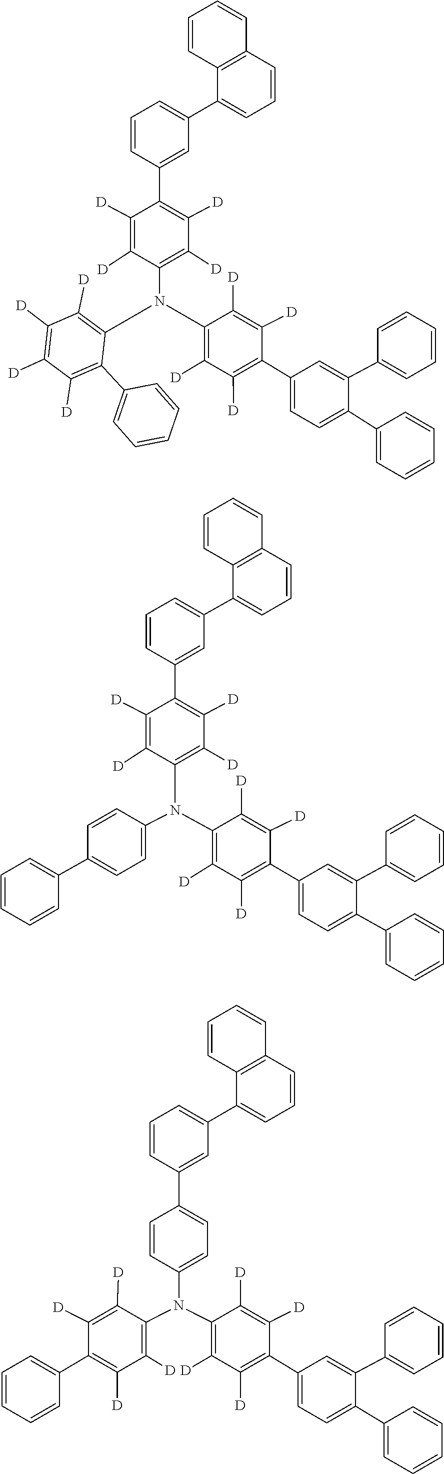 [SMILES: [2H]c1c([2H])c([2H])c(N(c2c([2H])c([2H])c(-c3cccc(-c4cccc5ccccc45)c3)c([2H])c2[2H])c2c([2H])c([2H])c(-c3ccc(-c4ccccc4)c(-c4ccccc4)c3)c([2H])c2[2H])c(-c2ccccc2)c1[2H] ; [2H]c1c([2H])c(N(c2ccc(-c3ccccc3)cc2)c2c([2H])c([2H])c(-c3ccc(-c4ccccc4)c(-c4ccccc4)c3)c([2H])c2[2H])c([2H])c([2H])c1-c1cccc(-c2cccc3ccccc23)c1 ; [2H]c1c([2H])c(N(c2ccc(-c3cccc(-c4cccc5ccccc45)c3)cc2)c2c([2H])c([2H])c(-c3ccc(-c4ccccc4)c(-c4ccccc4)c3)c([2H])c2[2H])c([2H])c([2H])c1-c1ccccc1]

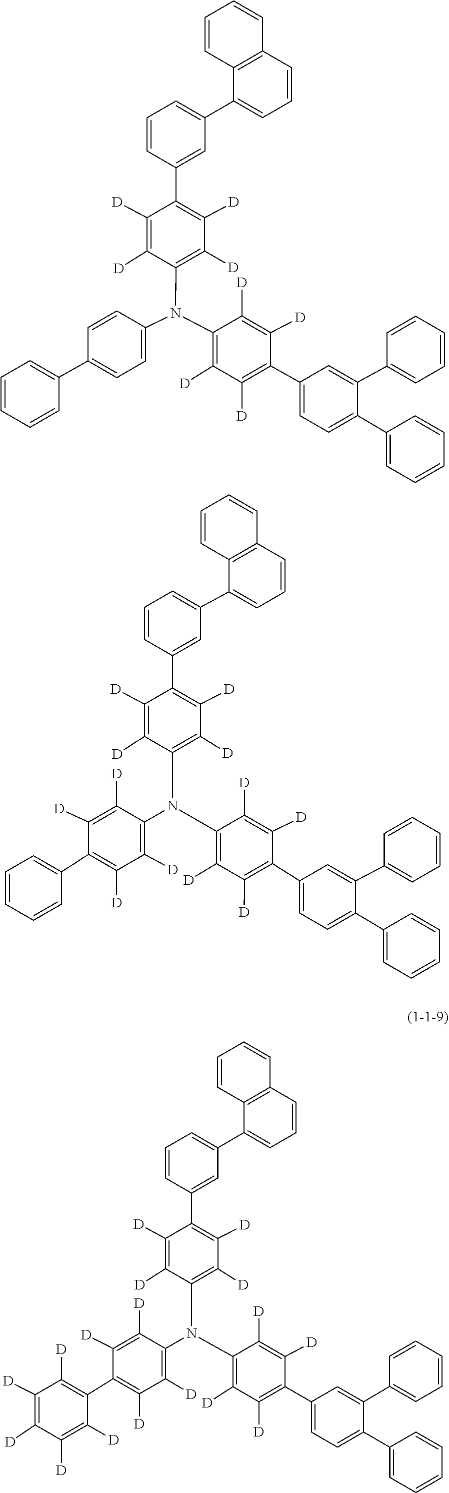

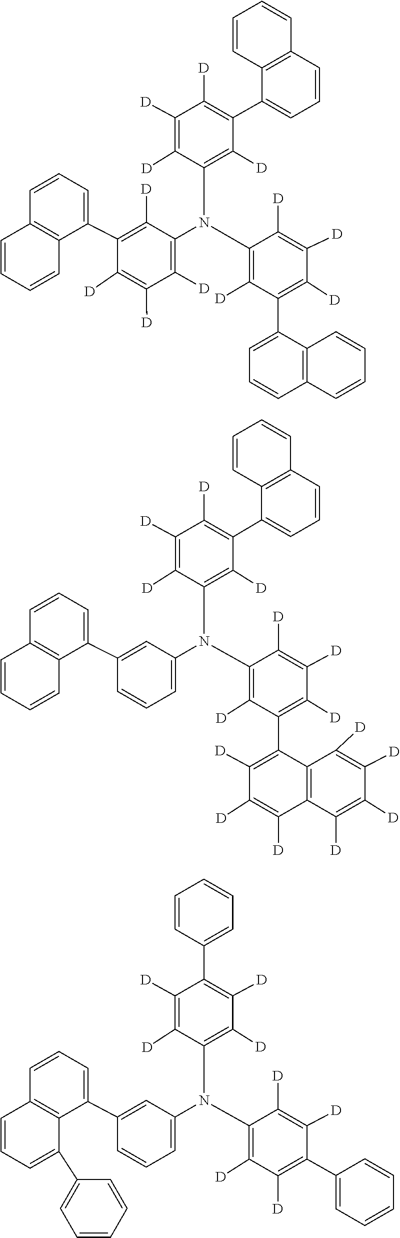

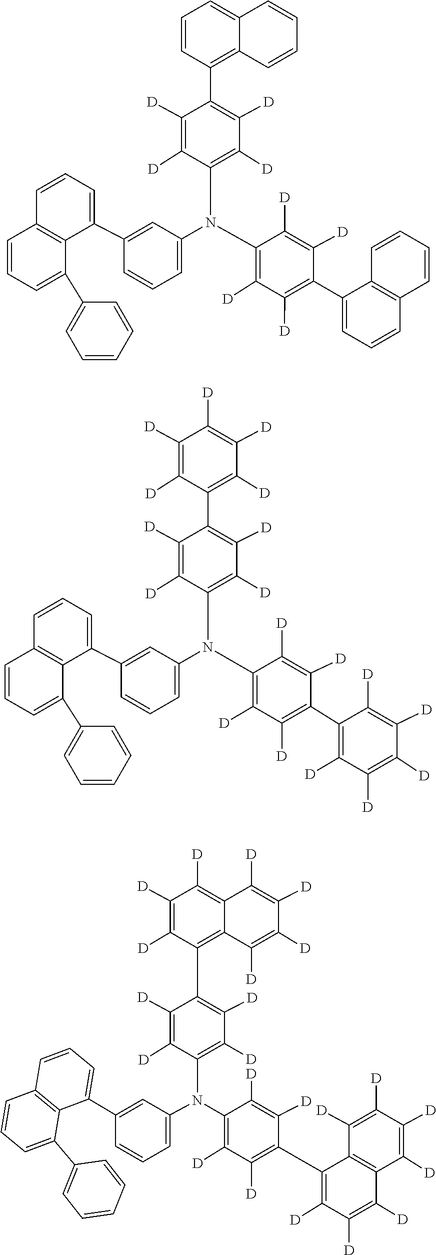

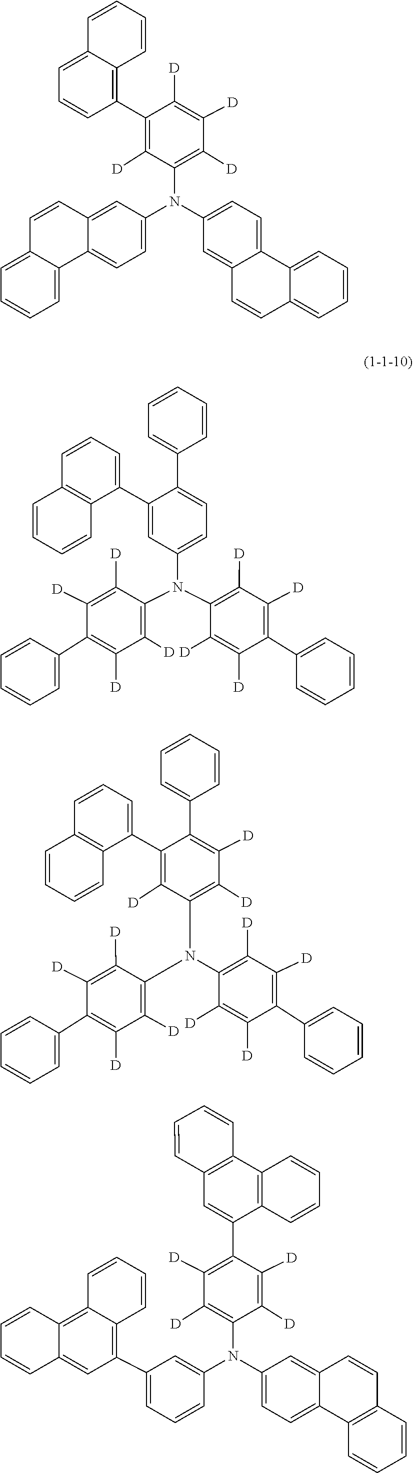 [SMILES: [2H]c1c([2H])c(-c2cccc3ccccc23)c([2H])c(N(c2ccc3c(ccc4ccccc43)c2)c2ccc3c(ccc4ccccc43)c2)c1[2H] ; [2H]c1c([2H])c(N(c2ccc(-c3ccccc3)c(-c3cccc4ccccc34)c2)c2c([2H])c([2H])c(-c3ccccc3)c([2H])c2[2H])c([2H])c([2H])c1-c1ccccc1 ; [2H]c1c([2H])c(N(c2c([2H])c([2H])c(-c3ccccc3)c([2H])c2[2H])c2c([2H])c([2H])c(-c3ccccc3)c(-c3cccc4ccccc34)c2[2H])c([2H])c([2H])c1-c1ccccc1 ; [2H]c1c([2H])c(N(c2cccc(-c3cc4ccccc4c4ccccc34)c2)c2ccc3c(ccc4ccccc43)c2)c([2H])c([2H])c1-c1cc2ccccc2c2ccccc12]

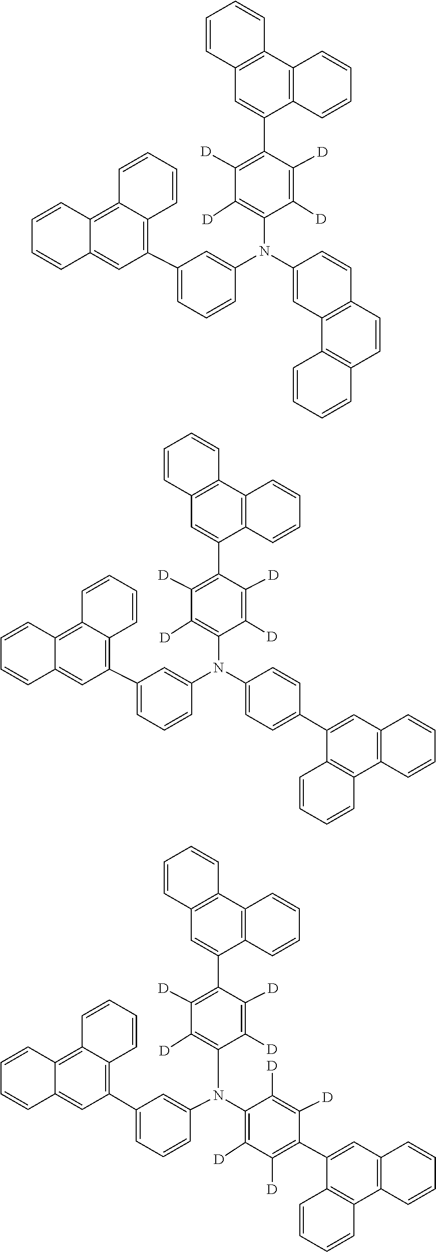 [SMILES: [2H]c1c([2H])c(N(c2cccc(-c3cc4ccccc4c4ccccc34)c2)c2ccc3ccc4ccccc4c3c2)c([2H])c([2H])c1-c1cc2ccccc2c2ccccc12 ; [2H]c1c([2H])c(N(c2ccc(-c3cc4ccccc4c4ccccc34)cc2)c2cccc(-c3cc4ccccc4c4ccccc34)c2)c([2H])c([2H])c1-c1cc2ccccc2c2ccccc12 ; [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1cc2ccccc2c2ccccc12]

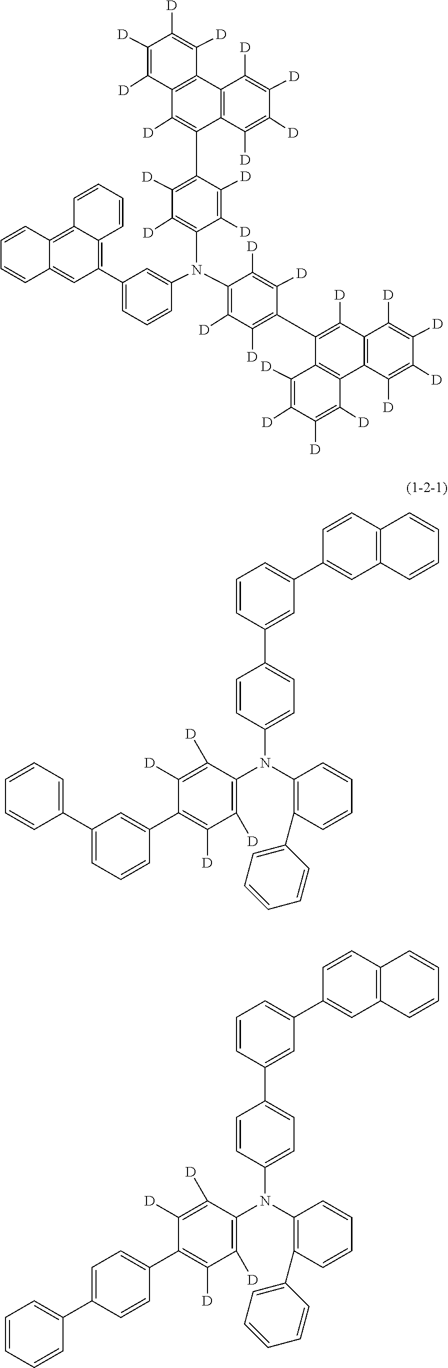 [SMILES: [2H]c1c([2H])c(N(c2cccc(-c3cc4ccccc4c4ccccc34)c2)c2c([2H])c([2H])c(-c3c([2H])c4c([2H])c([2H])c([2H])c([2H])c4c4c([2H])c([2H])c([2H])c([2H])c34)c([2H])c2[2H])c([2H])c([2H])c1-c1c([2H])c2c([2H])c([2H])c([2H])c([2H])c2c2c([2H])c([2H])c([2H])c([2H])c12 ; [2H]c1c([2H])c(N(c2ccc(-c3cccc(-c4ccc5ccccc5c4)c3)cc2)c2ccccc2-c2ccccc2)c([2H])c([2H])c1-c1cccc(-c2ccccc2)c1 ; [2H]c1c([2H])c(N(c2ccc(-c3cccc(-c4ccc5ccccc5c4)c3)cc2)c2ccccc2-c2ccccc2)c([2H])c([2H])c1-c1ccc(-c2ccccc2)cc1]

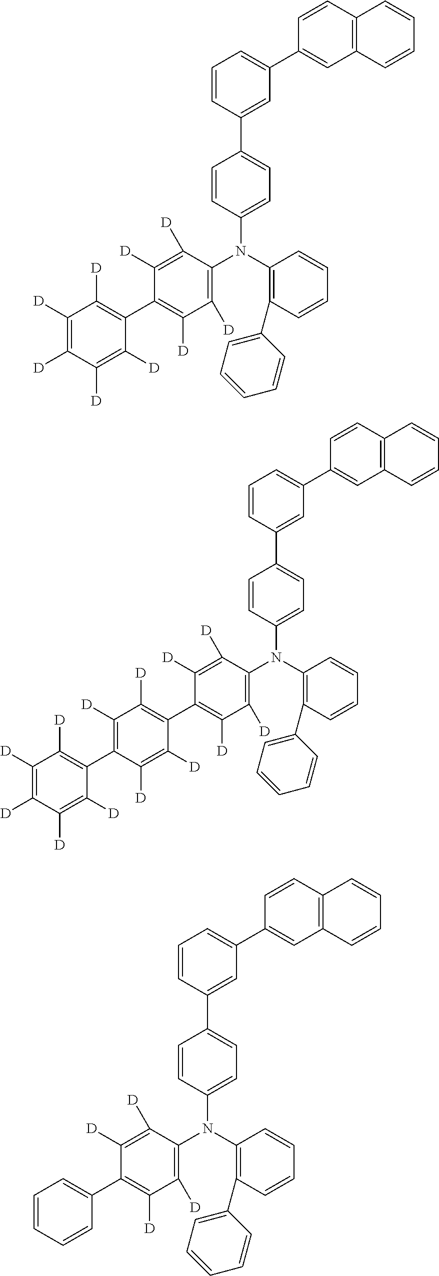 [SMILES: [2H]c1c([2H])c([2H])c(-c2c([2H])c([2H])c(N(c3ccc(-c4cccc(-c5ccc6ccccc6c5)c4)cc3)c3ccccc3-c3ccccc3)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccc7ccccc7c6)c5)cc4)c4ccccc4-c4ccccc4)c([2H])c3[2H])c([2H])c2[2H])c([2H])c1[2H] ; [2H]c1c([2H])c(N(c2ccc(-c3cccc(-c4ccc5ccccc5c4)c3)cc2)c2ccccc2-c2ccccc2)c([2H])c([2H])c1-c1ccccc1]

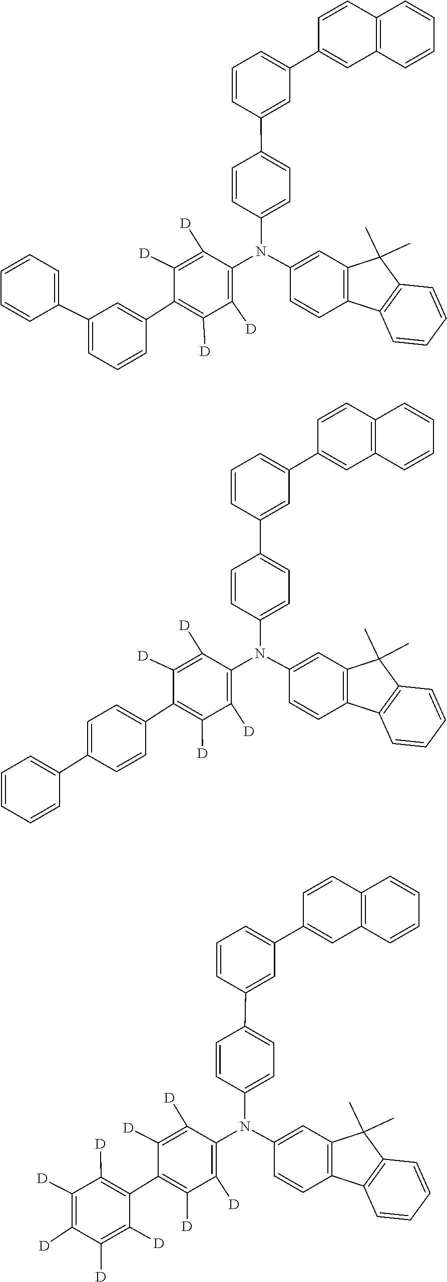 [SMILES: [2H]c1c([2H])c(N(c2ccc(-c3cccc(-c4ccc5ccccc5c4)c3)cc2)c2ccc3c(c2)C(C)(C)c2ccccc2-3)c([2H])c([2H])c1-c1cccc(-c2ccccc2)c1 ; [2H]c1c([2H])c(N(c2ccc(-c3cccc(-c4ccc5ccccc5c4)c3)cc2)c2ccc3c(c2)C(C)(C)c2ccccc2-3)c([2H])c([2H])c1-c1ccc(-c2ccccc2)cc1 ; [2H]c1c([2H])c([2H])c(-c2c([2H])c([2H])c(N(c3ccc(-c4cccc(-c5ccc6ccccc6c5)c4)cc3)c3ccc4c(c3)C(C)(C)c3ccccc3-4)c([2H])c2[2H])c([2H])c1[2H]]

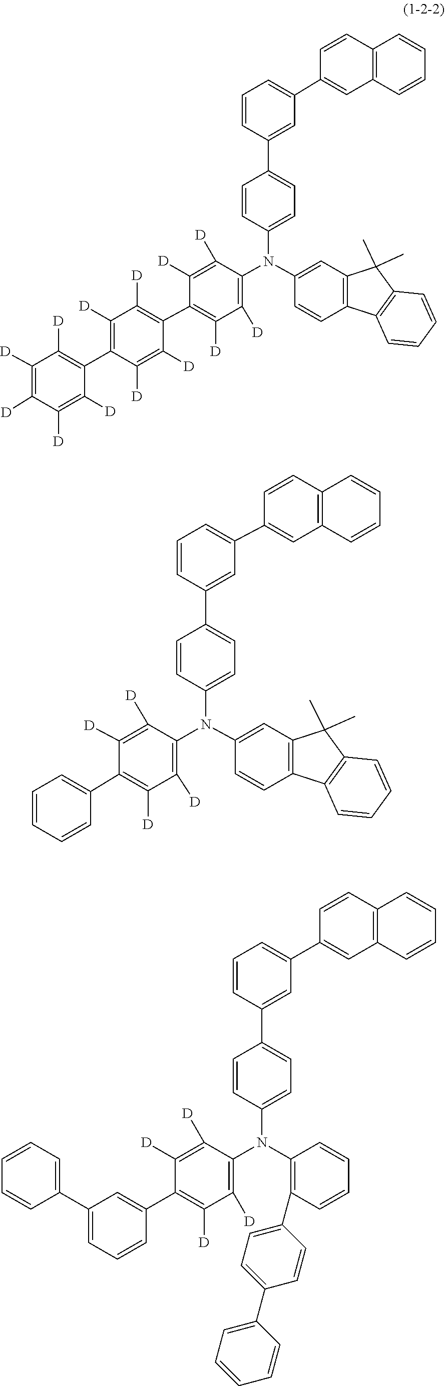 [SMILES: [2H]c1c([2H])c([2H])c(-c2c([2H])c([2H])c(-c3c([2H])c([2H])c(N(c4ccc(-c5cccc(-c6ccc7ccccc7c6)c5)cc4)c4ccc5c(c4)C(C)(C)c4ccccc4-5)c([2H])c3[2H])c([2H])c2[2H])c([2H])c1[2H] ; [2H]c1c([2H])c(N(c2ccc(-c3cccc(-c4ccc5ccccc5c4)c3)cc2)c2ccc3c(c2)C(C)(C)c2ccccc2-3)c([2H])c([2H])c1-c1ccccc1 ; [2H]c1c([2H])c(N(c2ccc(-c3cccc(-c4ccc5ccccc5c4)c3)cc2)c2ccccc2-c2ccc(-c3ccccc3)cc2)c([2H])c([2H])c1-c1cccc(-c2ccccc2)c1]

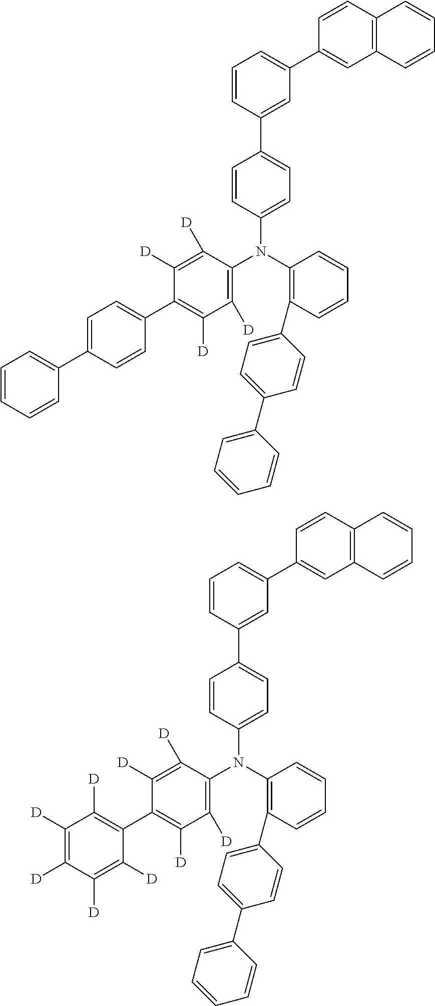 [SMILES: [2H]c1c([2H])c(N(c2ccc(-c3cccc(-c4ccc5ccccc5c4)c3)cc2)c2ccccc2-c2ccc(-c3ccccc3)cc2)c([2H])c([2H])c1-c1ccc(-c2ccccc2)cc1 ; [2H]c1c([2H])c([2H])c(-c2c([2H])c([2H])c(N(c3ccc(-c4cccc(-c5ccc6ccccc6c5)c4)cc3)c3ccccc3-c3ccc(-c4ccccc4)cc3)c([2H])c2[2H])c([2H])c1[2H]]

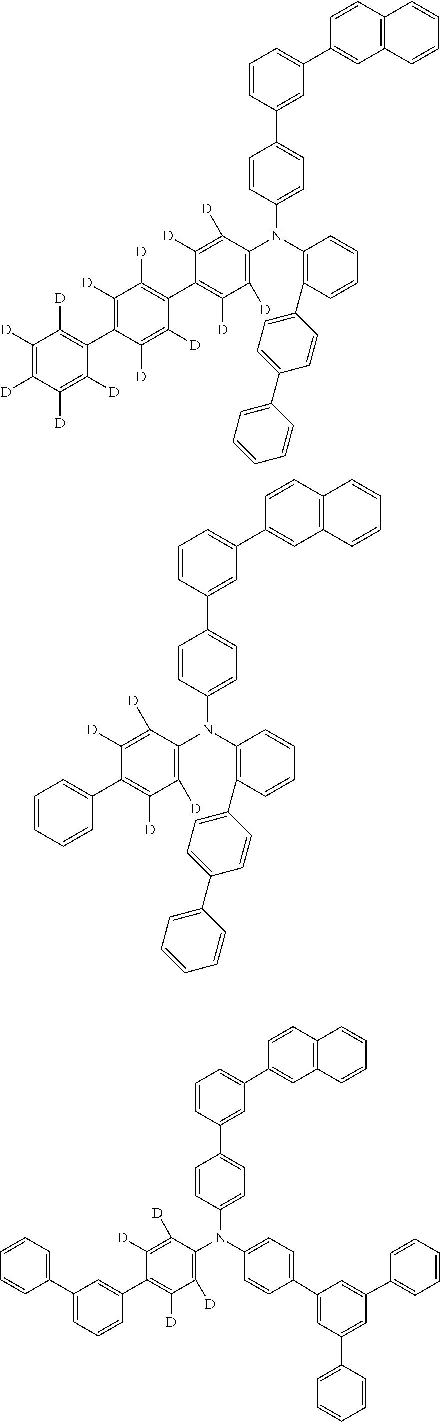

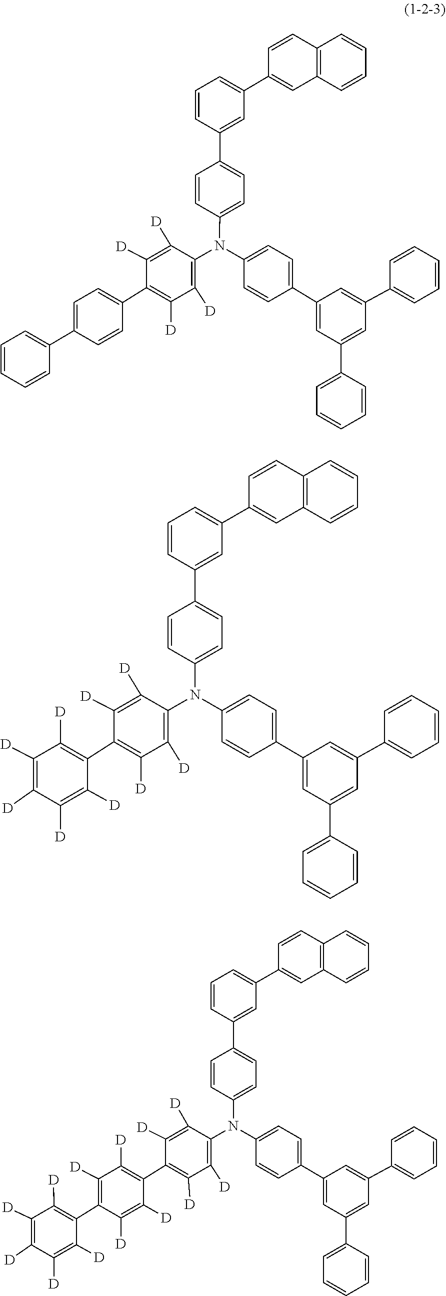

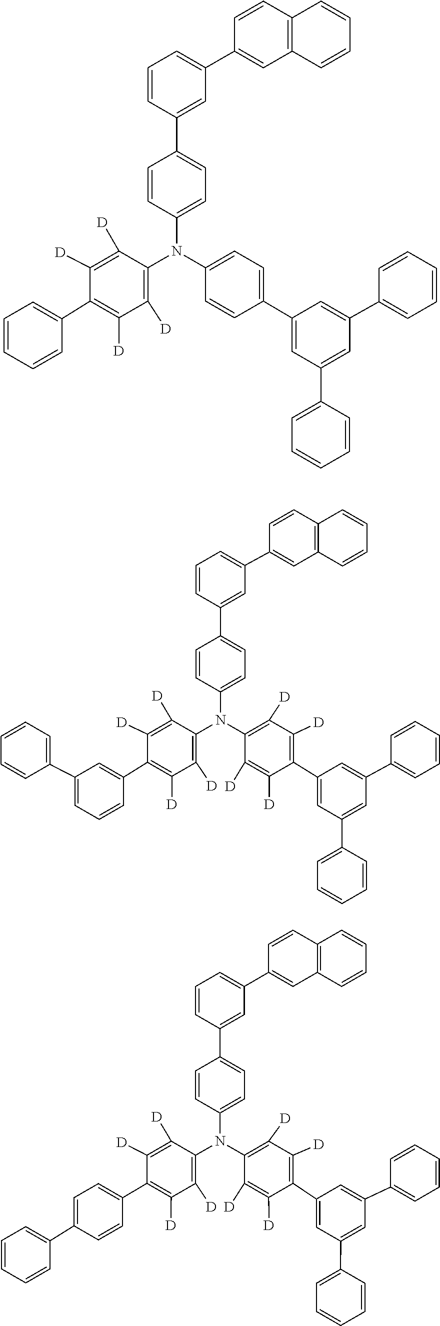

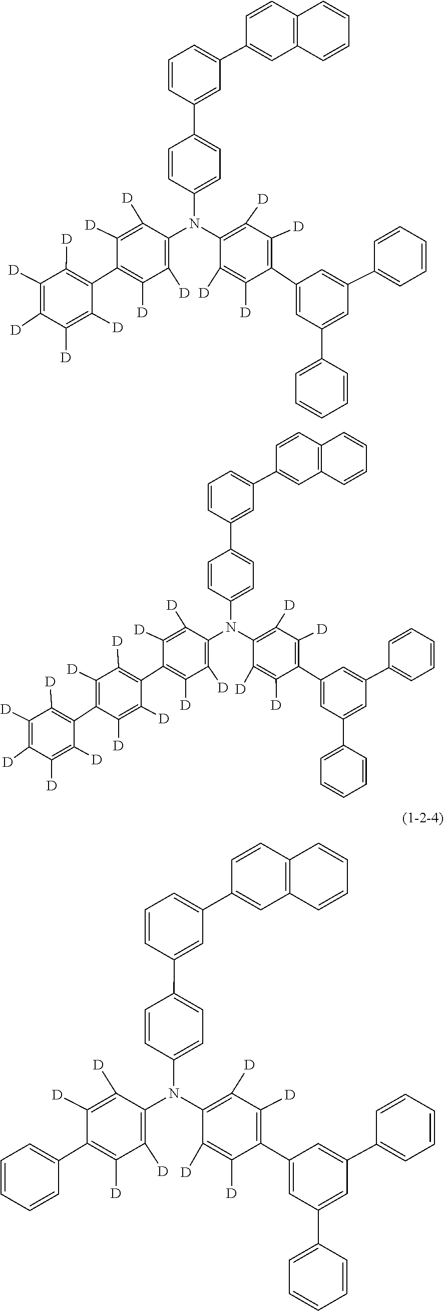

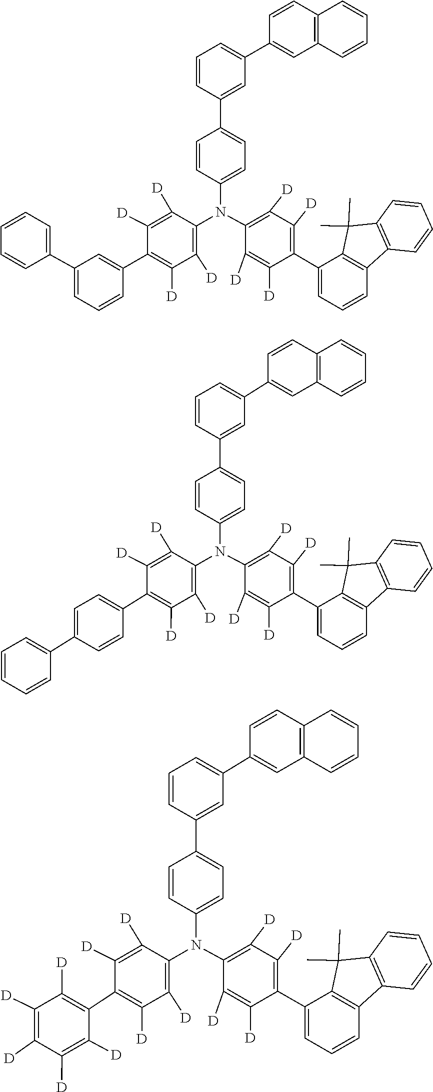

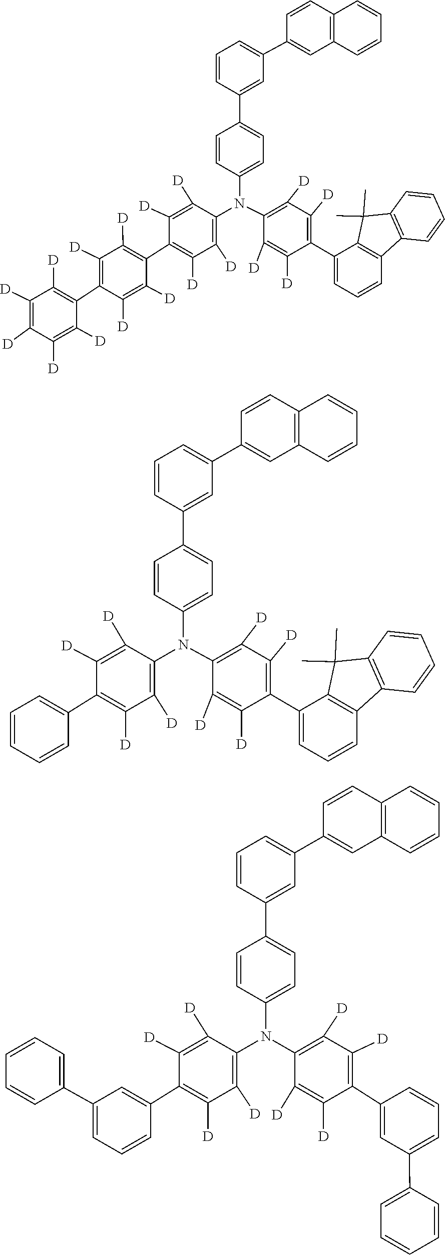

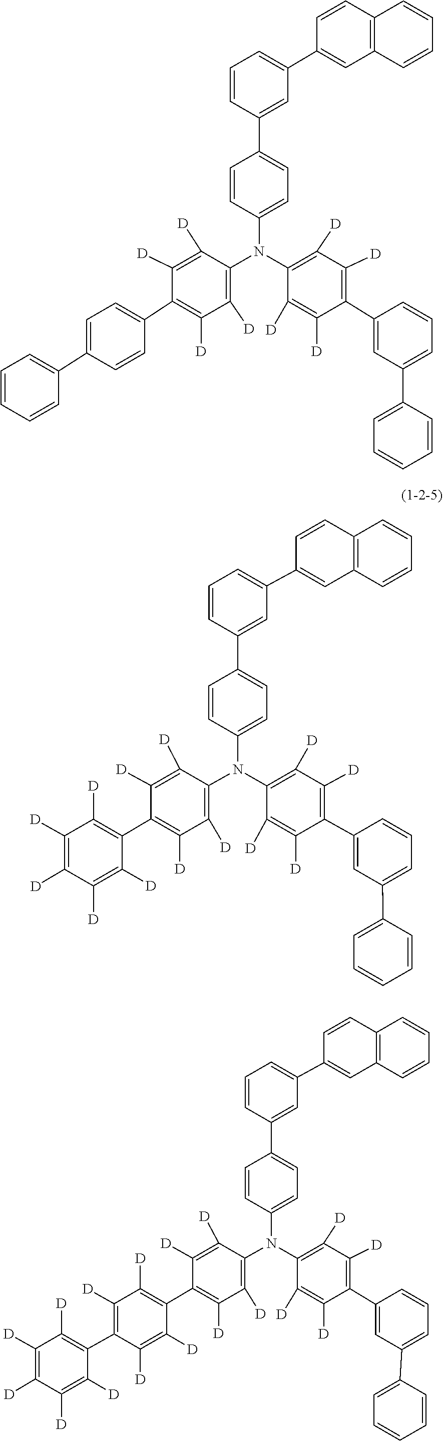

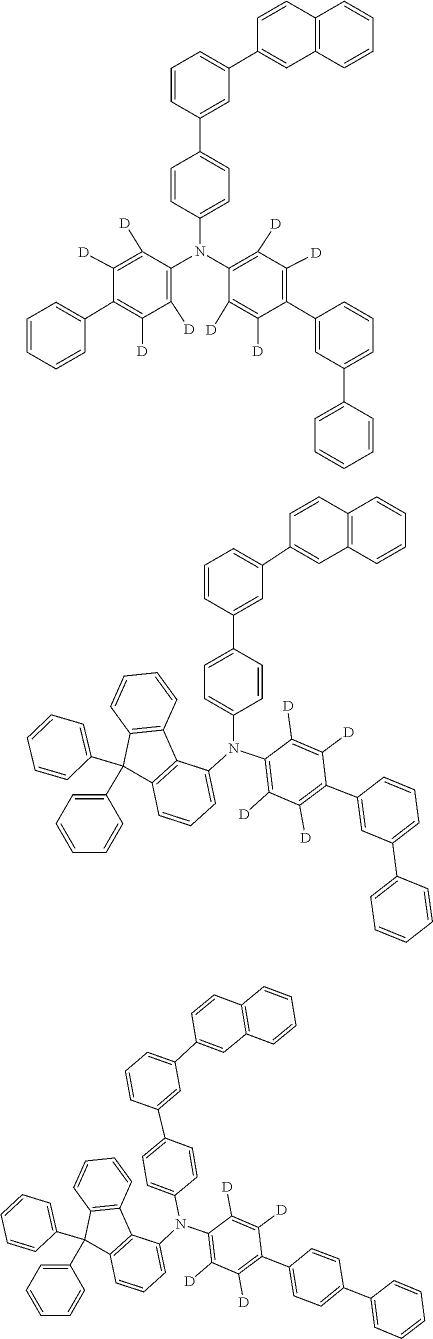

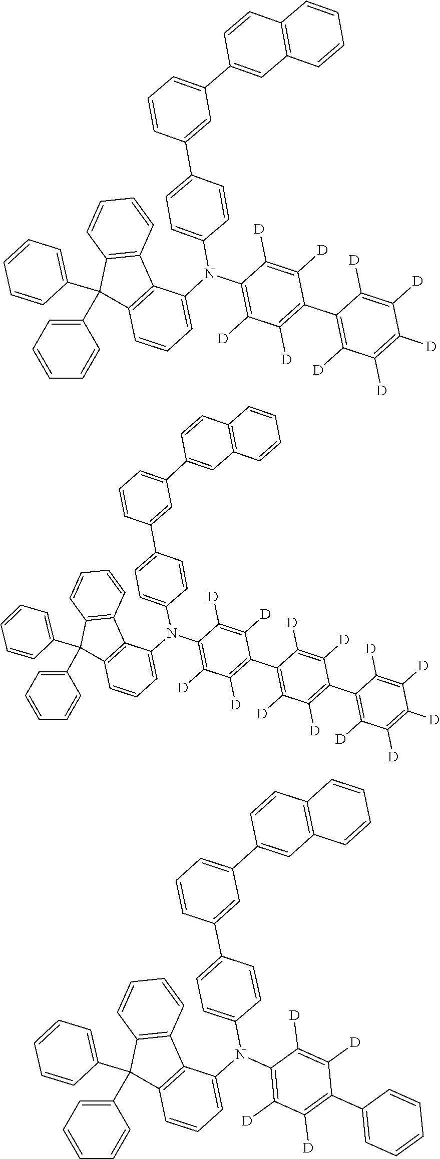

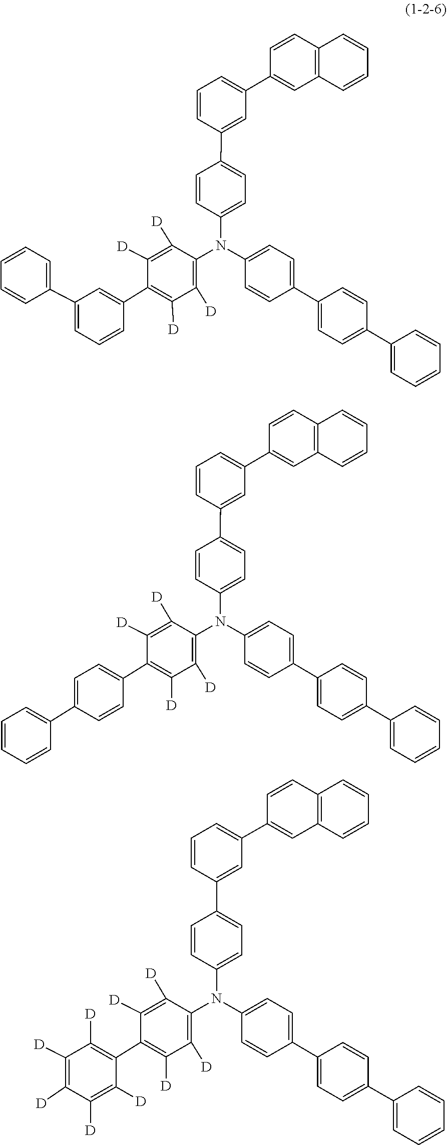 [SMILES: [2H]c1c([2H])c(N(c2ccc(-c3ccc(-c4ccccc4)cc3)cc2)c2ccc(-c3cccc(-c4ccc5ccccc5c4)c3)cc2)c([2H])c([2H])c1-c1cccc(-c2ccccc2)c1 ; [2H]c1c([2H])c(N(c2ccc(-c3ccc(-c4ccccc4)cc3)cc2)c2ccc(-c3cccc(-c4ccc5ccccc5c4)c3)cc2)c([2H])c([2H])c1-c1ccc(-c2ccccc2)cc1 ; [2H]c1c([2H])c([2H])c(-c2c([2H])c([2H])c(N(c3ccc(-c4ccc(-c5ccccc5)cc4)cc3)c3ccc(-c4cccc(-c5ccc6ccccc6c5)c4)cc3)c([2H])c2[2H])c([2H])c1[2H]]

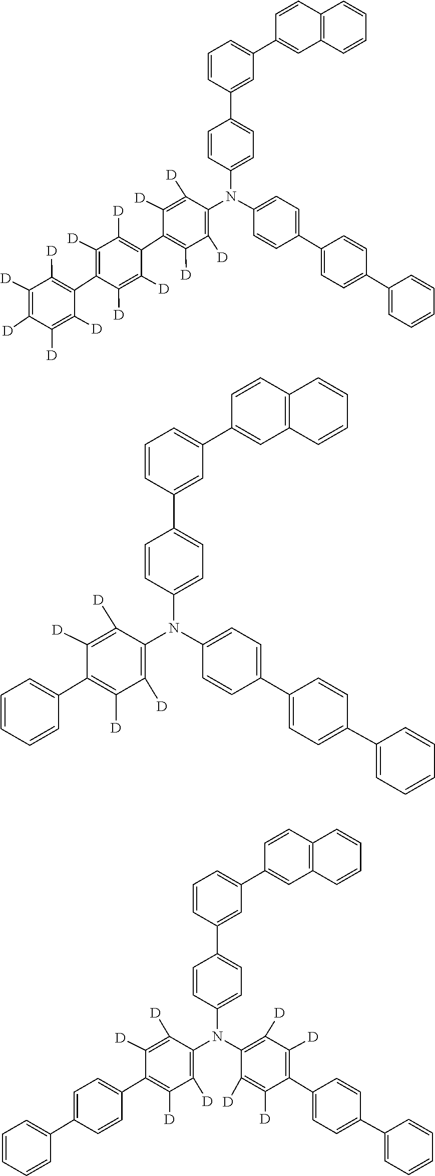 [SMILES: [2H]c1c([2H])c([2H])c(-c2c([2H])c([2H])c(-c3c([2H])c([2H])c(N(c4ccc(-c5ccc(-c6ccccc6)cc5)cc4)c4ccc(-c5cccc(-c6ccc7ccccc7c6)c5)cc4)c([2H])c3[2H])c([2H])c2[2H])c([2H])c1[2H] ; [2H]c1c([2H])c(N(c2ccc(-c3ccc(-c4ccccc4)cc3)cc2)c2ccc(-c3cccc(-c4ccc5ccccc5c4)c3)cc2)c([2H])c([2H])c1-c1ccccc1 ; [2H]c1c([2H])c(N(c2ccc(-c3cccc(-c4ccc5ccccc5c4)c3)cc2)c2c([2H])c([2H])c(-c3ccc(-c4ccccc4)cc3)c([2H])c2[2H])c([2H])c([2H])c1-c1ccc(-c2ccccc2)cc1]

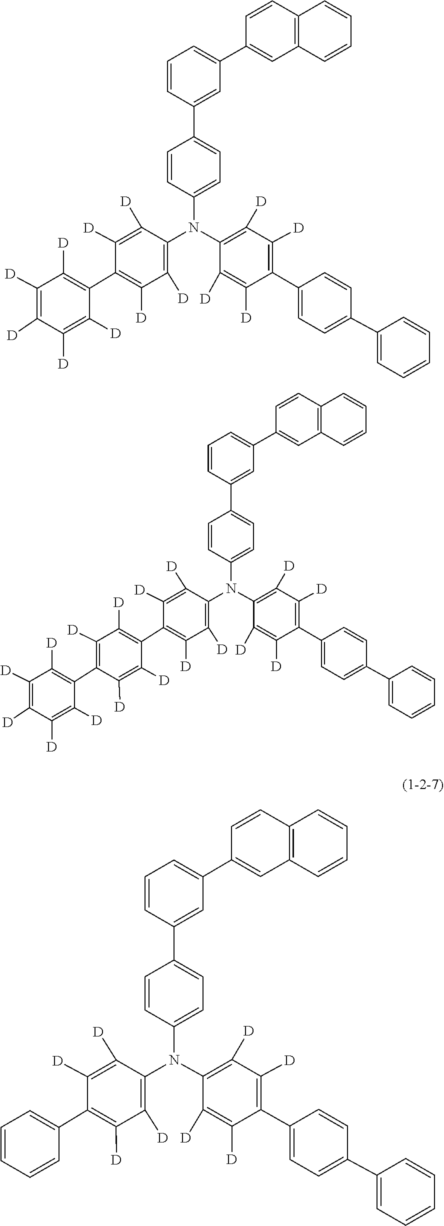 [SMILES: [2H]c1c([2H])c([2H])c(-c2c([2H])c([2H])c(N(c3ccc(-c4cccc(-c5ccc6ccccc6c5)c4)cc3)c3c([2H])c([2H])c(-c4ccc(-c5ccccc5)cc4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccc7ccccc7c6)c5)cc4)c4c([2H])c([2H])c(-c5ccc(-c6ccccc6)cc5)c([2H])c4[2H])c([2H])c3[2H])c([2H])c2[2H])c([2H])c1[2H] ; [2H]c1c([2H])c(N(c2ccc(-c3cccc(-c4ccc5ccccc5c4)c3)cc2)c2c([2H])c([2H])c(-c3ccc(-c4ccccc4)cc3)c([2H])c2[2H])c([2H])c([2H])c1-c1ccccc1]

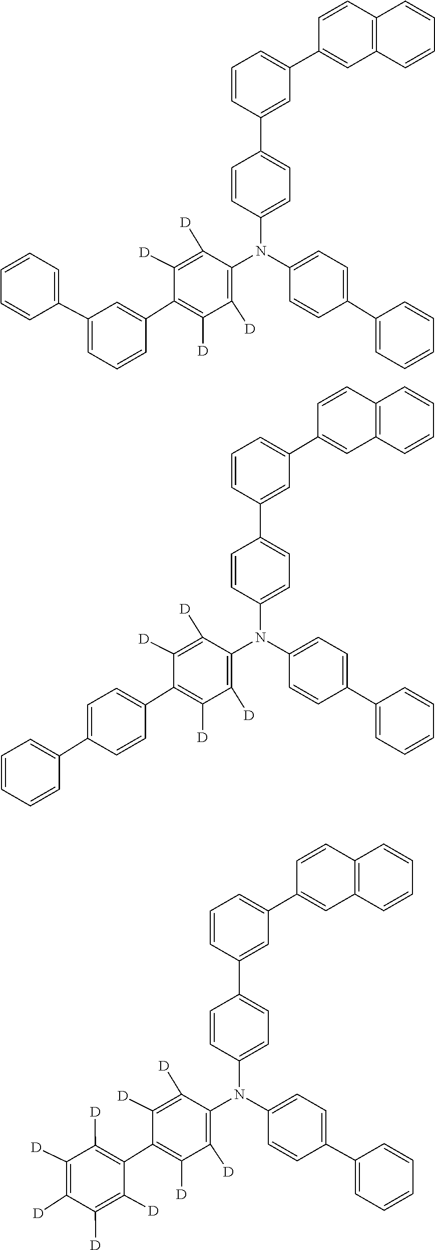

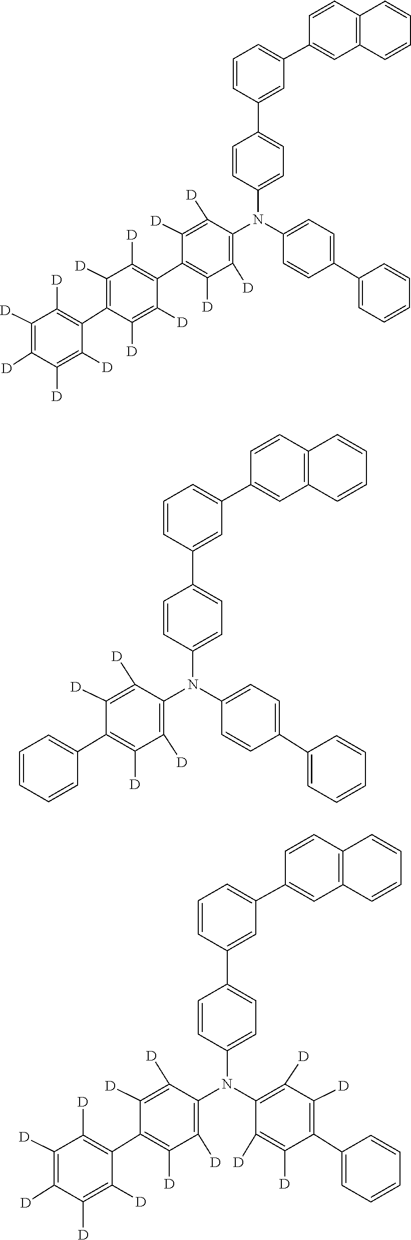

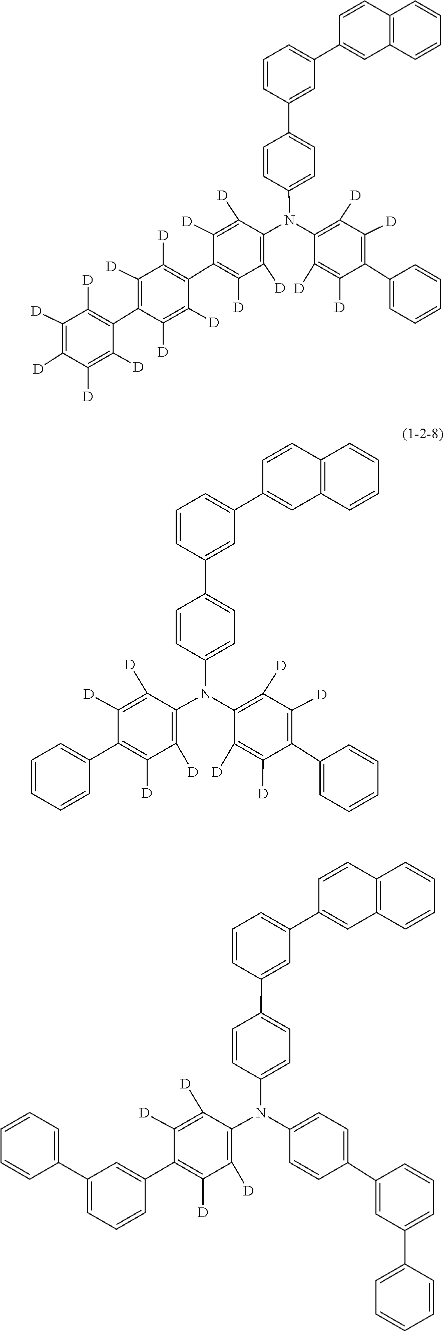 [SMILES: [2H]c1c([2H])c([2H])c(-c2c([2H])c([2H])c(-c3c([2H])c([2H])c(N(c4ccc(-c5cccc(-c6ccc7ccccc7c6)c5)cc4)c4c([2H])c([2H])c(-c5ccccc5)c([2H])c4[2H])c([2H])c3[2H])c([2H])c2[2H])c([2H])c1[2H] ; [2H]c1c([2H])c(N(c2ccc(-c3cccc(-c4ccc5ccccc5c4)c3)cc2)c2c([2H])c([2H])c(-c3ccccc3)c([2H])c2[2H])c([2H])c([2H])c1-c1ccccc1 ; [2H]c1c([2H])c(N(c2ccc(-c3cccc(-c4ccccc4)c3)cc2)c2ccc(-c3cccc(-c4ccc5ccccc5c4)c3)cc2)c([2H])c([2H])c1-c1cccc(-c2ccccc2)c1]

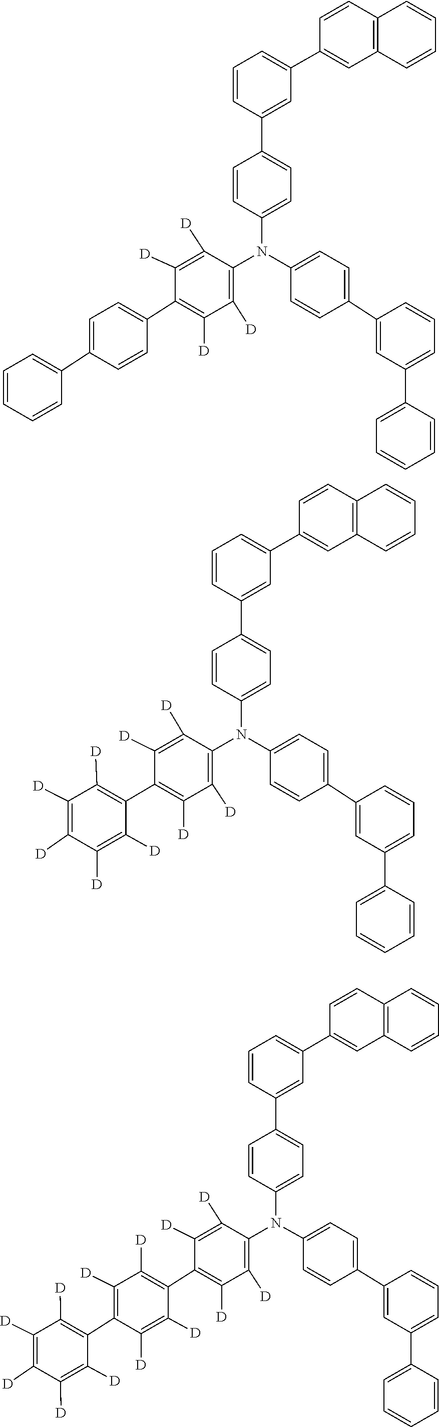

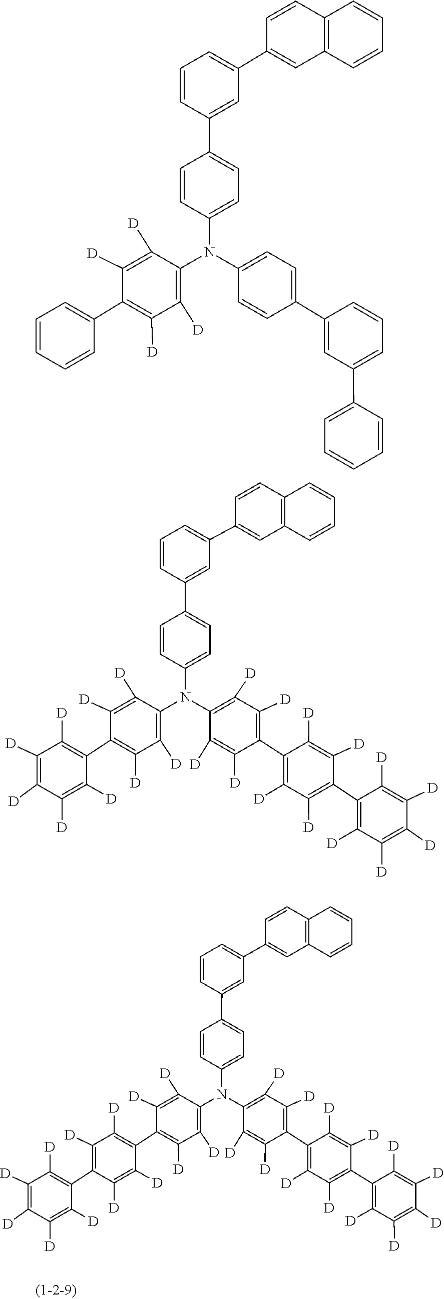

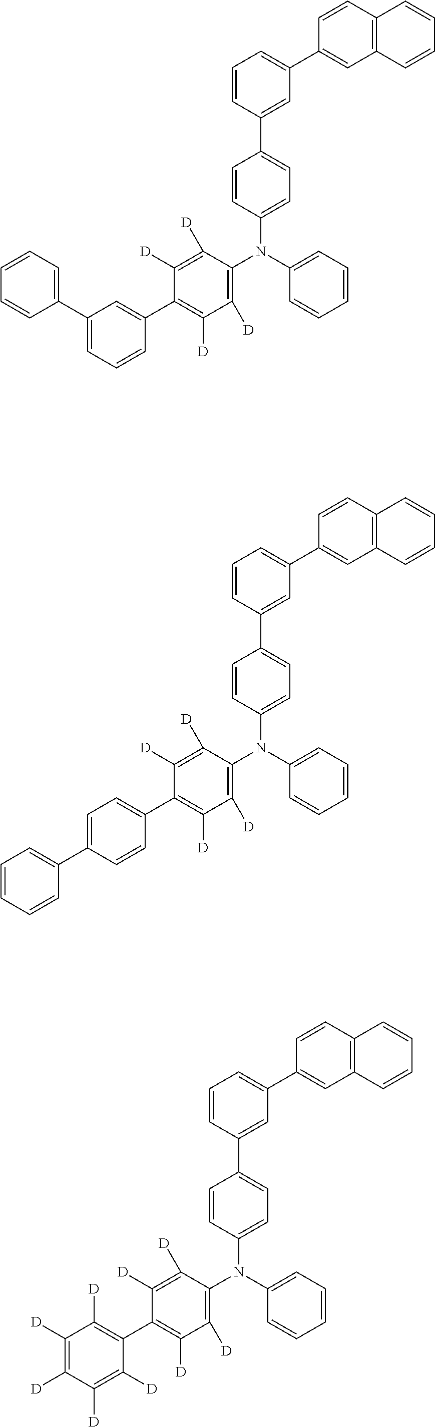 [SMILES: [2H]c1c([2H])c(N(c2ccccc2)c2ccc(-c3cccc(-c4ccc5ccccc5c4)c3)cc2)c([2H])c([2H])c1-c1cccc(-c2ccccc2)c1 ; [2H]c1c([2H])c(N(c2ccccc2)c2ccc(-c3cccc(-c4ccc5ccccc5c4)c3)cc2)c([2H])c([2H])c1-c1ccc(-c2ccccc2)cc1 ; [2H]c1c([2H])c([2H])c(-c2c([2H])c([2H])c(N(c3ccccc3)c3ccc(-c4cccc(-c5ccc6ccccc6c5)c4)cc3)c([2H])c2[2H])c([2H])c1[2H]]

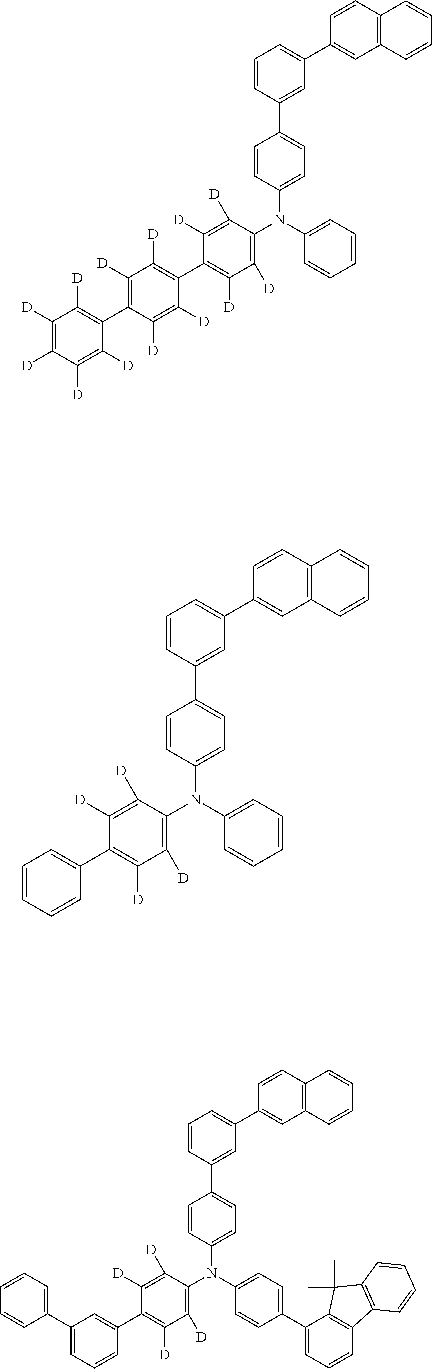 [SMILES: [2H]c1c([2H])c([2H])c(-c2c([2H])c([2H])c(-c3c([2H])c([2H])c(N(c4ccccc4)c4ccc(-c5cccc(-c6ccc7ccccc7c6)c5)cc4)c([2H])c3[2H])c([2H])c2[2H])c([2H])c1[2H] ; [2H]c1c([2H])c(N(c2ccccc2)c2ccc(-c3cccc(-c4ccc5ccccc5c4)c3)cc2)c([2H])c([2H])c1-c1ccccc1 ; [2H]c1c([2H])c(N(c2ccc(-c3cccc(-c4ccc5ccccc5c4)c3)cc2)c2ccc(-c3cccc4c3C(C)(C)c3ccccc3-4)cc2)c([2H])c([2H])c1-c1cccc(-c2ccccc2)c1]

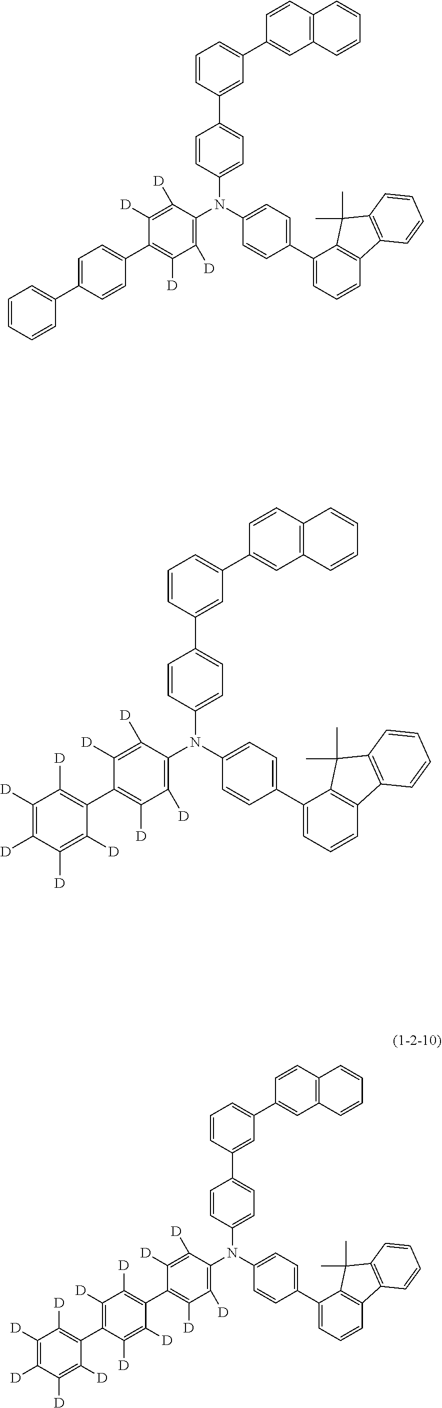 [SMILES: [2H]c1c([2H])c(N(c2ccc(-c3cccc(-c4ccc5ccccc5c4)c3)cc2)c2ccc(-c3cccc4c3C(C)(C)c3ccccc3-4)cc2)c([2H])c([2H])c1-c1ccc(-c2ccccc2)cc1 ; [2H]c1c([2H])c([2H])c(-c2c([2H])c([2H])c(N(c3ccc(-c4cccc(-c5ccc6ccccc6c5)c4)cc3)c3ccc(-c4cccc5c4C(C)(C)c4ccccc4-5)cc3)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccc7ccccc7c6)c5)cc4)c4ccc(-c5cccc6c5C(C)(C)c5ccccc5-6)cc4)c([2H])c3[2H])c([2H])c2[2H])c([2H])c1[2H]]

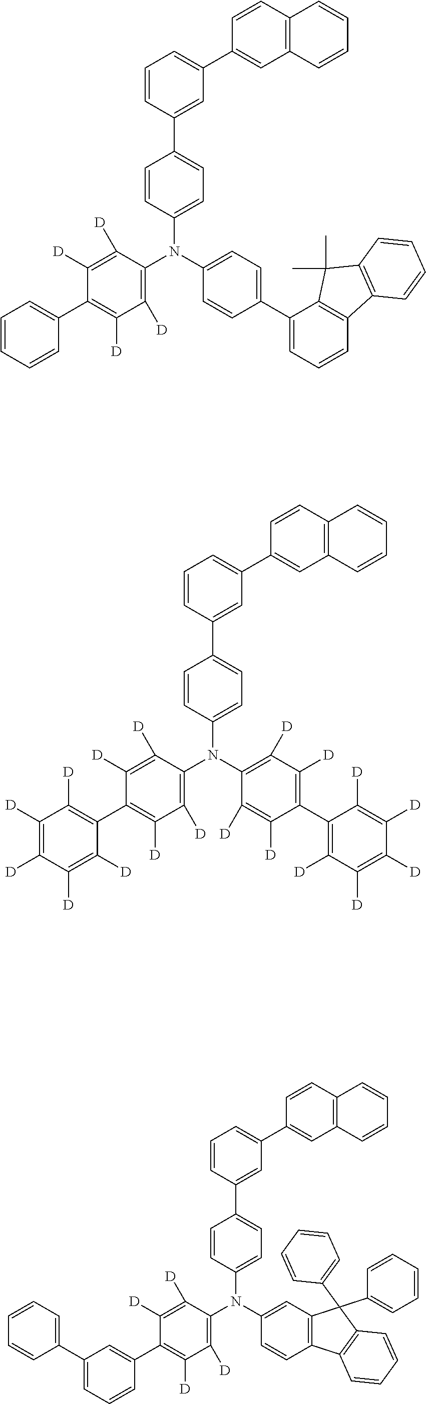 [SMILES: [2H]c1c([2H])c(N(c2ccc(-c3cccc(-c4ccc5ccccc5c4)c3)cc2)c2ccc(-c3cccc4c3C(C)(C)c3ccccc3-4)cc2)c([2H])c([2H])c1-c1ccccc1 ; [2H]c1c([2H])c([2H])c(-c2c([2H])c([2H])c(N(c3ccc(-c4cccc(-c5ccc6ccccc6c5)c4)cc3)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c(N(c2ccc(-c3cccc(-c4ccc5ccccc5c4)c3)cc2)c2ccc3c(c2)C(c2ccccc2)(c2ccccc2)c2ccccc2-3)c([2H])c([2H])c1-c1cccc(-c2ccccc2)c1]

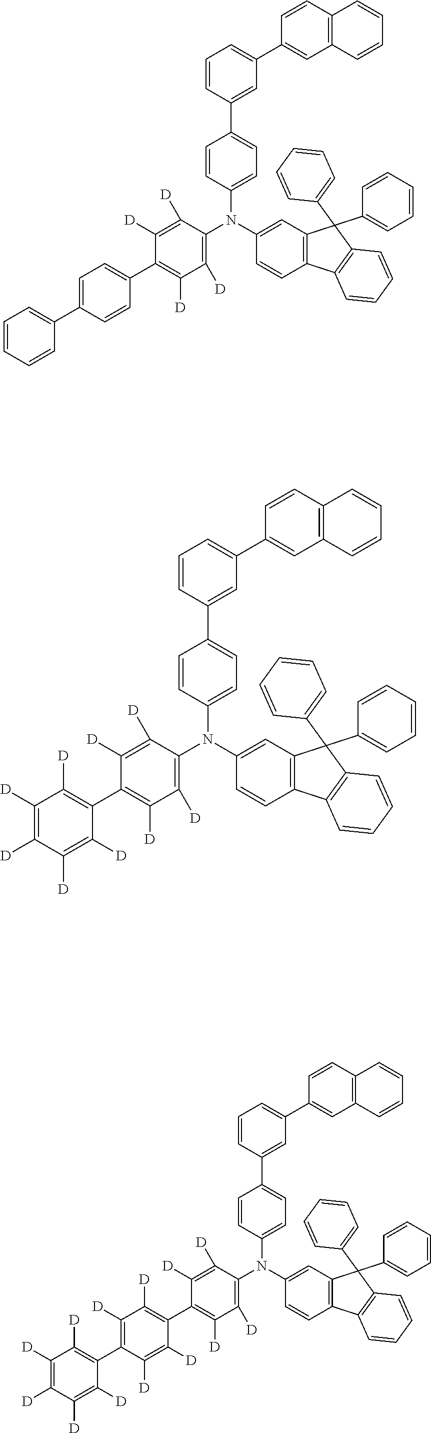 [SMILES: [2H]c1c([2H])c(N(c2ccc(-c3cccc(-c4ccc5ccccc5c4)c3)cc2)c2ccc3c(c2)C(c2ccccc2)(c2ccccc2)c2ccccc2-3)c([2H])c([2H])c1-c1ccc(-c2ccccc2)cc1 ; [2H]c1c([2H])c([2H])c(-c2c([2H])c([2H])c(N(c3ccc(-c4cccc(-c5ccc6ccccc6c5)c4)cc3)c3ccc4c(c3)C(c3ccccc3)(c3ccccc3)c3ccccc3-4)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccc7ccccc7c6)c5)cc4)c4ccc5c(c4)C(c4ccccc4)(c4ccccc4)c4ccccc4-5)c([2H])c3[2H])c([2H])c2[2H])c([2H])c1[2H]]

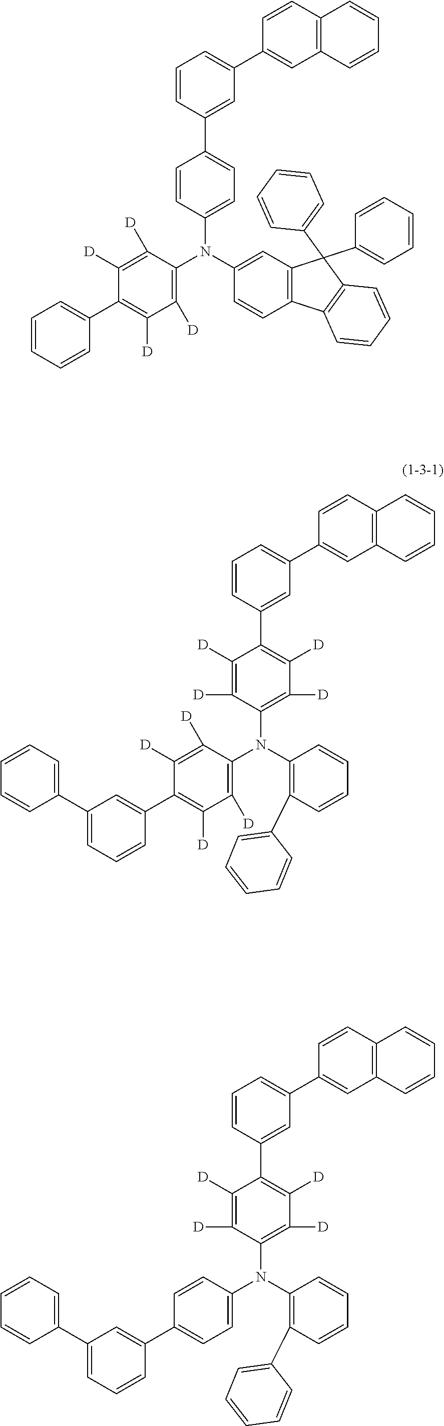 [SMILES: [2H]c1c([2H])c(N(c2ccc(-c3cccc(-c4ccc5ccccc5c4)c3)cc2)c2ccc3c(c2)C(c2ccccc2)(c2ccccc2)c2ccccc2-3)c([2H])c([2H])c1-c1ccccc1 ; [2H]c1c([2H])c(N(c2ccccc2-c2ccccc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2ccccc2-c2ccccc2)c([2H])c([2H])c1-c1cccc(-c2ccc3ccccc3c2)c1]

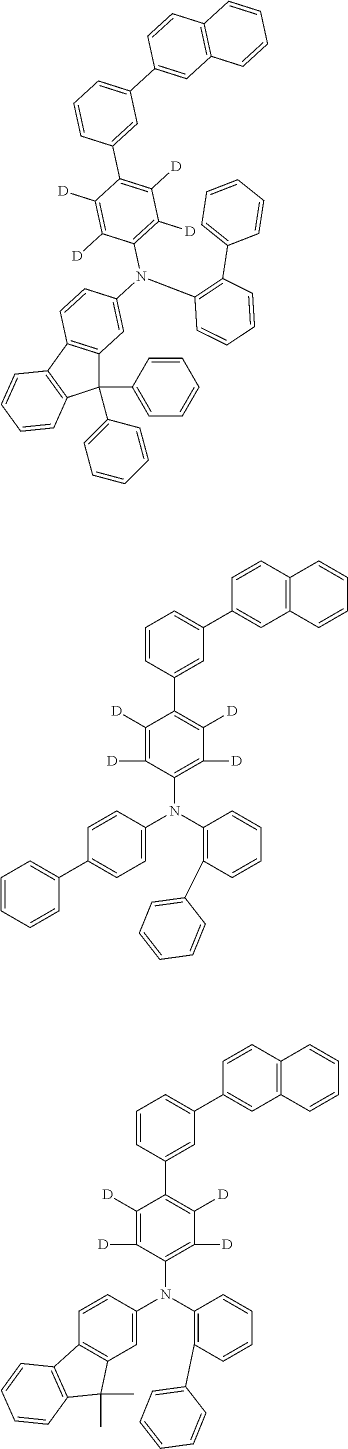 [SMILES: [2H]c1c([2H])c(N(c2ccc3c(c2)C(c2ccccc2)(c2ccccc2)c2ccccc2-3)c2ccccc2-c2ccccc2)c([2H])c([2H])c1-c1cccc(-c2ccc3ccccc3c2)c1 ; [2H]c1c([2H])c(N(c2ccc(-c3ccccc3)cc2)c2ccccc2-c2ccccc2)c([2H])c([2H])c1-c1cccc(-c2ccc3ccccc3c2)c1 ; [2H]c1c([2H])c(N(c2ccc3c(c2)C(C)(C)c2ccccc2-3)c2ccccc2-c2ccccc2)c([2H])c([2H])c1-c1cccc(-c2ccc3ccccc3c2)c1]

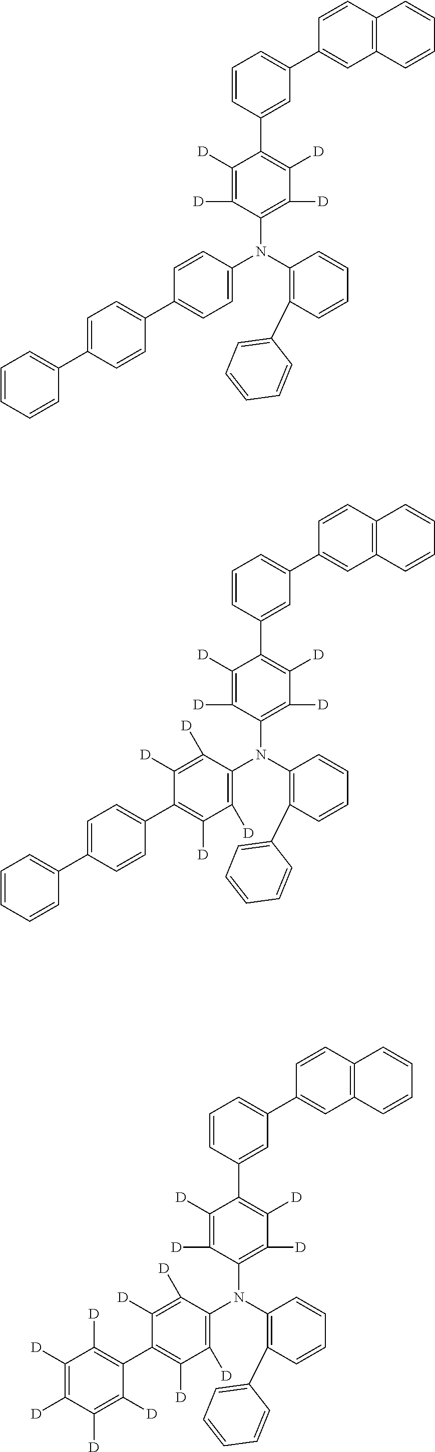 [SMILES: [2H]c1c([2H])c(N(c2ccc(-c3ccc(-c4ccccc4)cc3)cc2)c2ccccc2-c2ccccc2)c([2H])c([2H])c1-c1cccc(-c2ccc3ccccc3c2)c1 ; [2H]c1c([2H])c(N(c2ccccc2-c2ccccc2)c2c([2H])c([2H])c(-c3cccc(-c4ccc5ccccc5c4)c3)c([2H])c2[2H])c([2H])c([2H])c1-c1ccc(-c2ccccc2)cc1 ; [2H]c1c([2H])c([2H])c(-c2c([2H])c([2H])c(N(c3ccccc3-c3ccccc3)c3c([2H])c([2H])c(-c4cccc(-c5ccc6ccccc6c5)c4)c([2H])c3[2H])c([2H])c2[2H])c([2H])c1[2H]]

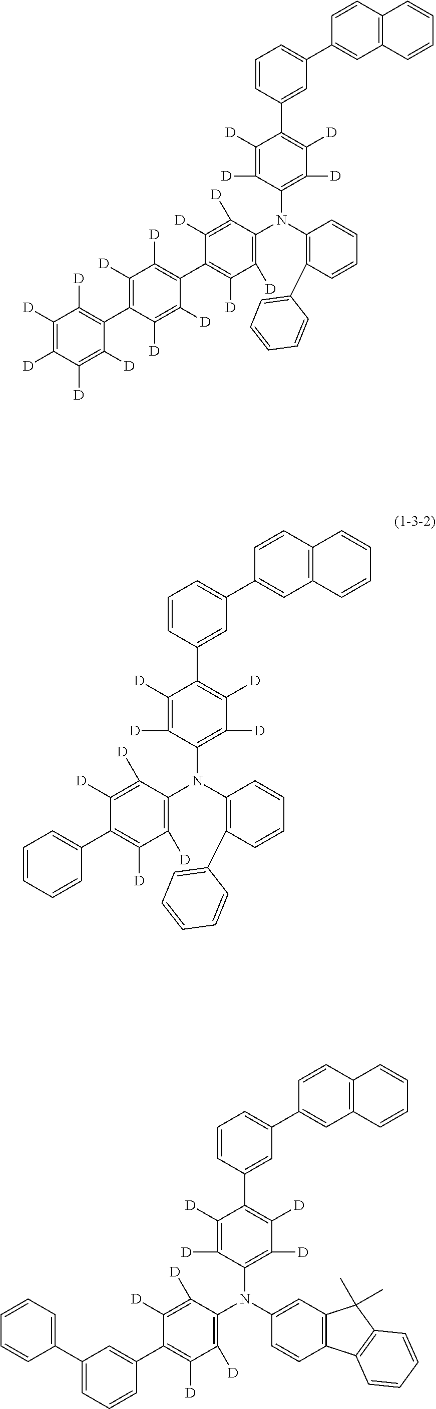 [SMILES: [2H]c1c([2H])c([2H])c(-c2c([2H])c([2H])c(-c3c([2H])c([2H])c(N(c4ccccc4-c4ccccc4)c4c([2H])c([2H])c(-c5cccc(-c6ccc7ccccc7c6)c5)c([2H])c4[2H])c([2H])c3[2H])c([2H])c2[2H])c([2H])c1[2H] ; [2H]c1c([2H])c(N(c2ccccc2-c2ccccc2)c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2ccc3c(c2)C(C)(C)c2ccccc2-3)c2c([2H])c([2H])c(-c3cccc(-c4ccc5ccccc5c4)c3)c([2H])c2[2H])c([2H])c([2H])c1-c1cccc(-c2ccccc2)c1]

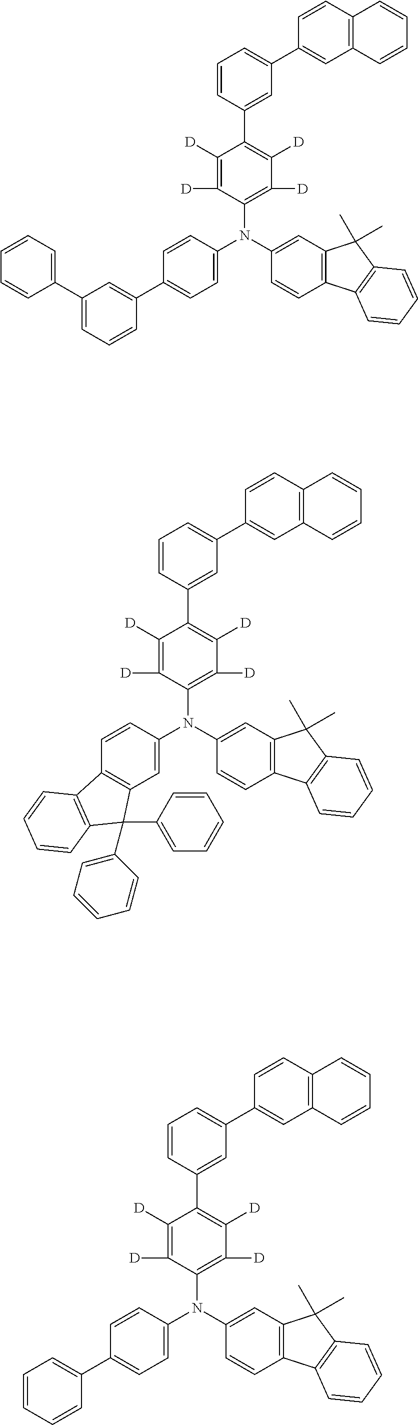 [SMILES: [2H]c1c([2H])c(N(c2ccc(-c3cccc(-c4ccccc4)c3)cc2)c2ccc3c(c2)C(C)(C)c2ccccc2-3)c([2H])c([2H])c1-c1cccc(-c2ccc3ccccc3c2)c1 ; [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1cccc(-c2ccc3ccccc3c2)c1 ; [2H]c1c([2H])c(N(c2ccc(-c3ccccc3)cc2)c2ccc3c(c2)C(C)(C)c2ccccc2-3)c([2H])c([2H])c1-c1cccc(-c2ccc3ccccc3c2)c1]

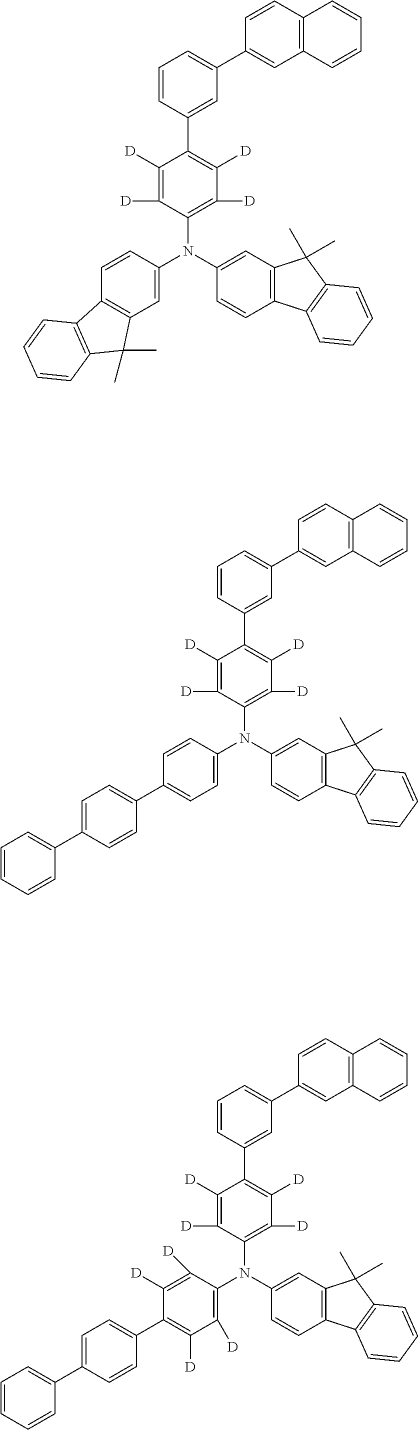 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1cccc(-c2ccc3ccccc3c2)c1 ; [2H]c1c([2H])c(N(c2ccc(-c3ccc(-c4ccccc4)cc3)cc2)c2ccc3c(c2)C(C)(C)c2ccccc2-3)c([2H])c([2H])c1-c1cccc(-c2ccc3ccccc3c2)c1 ; [2H]c1c([2H])c(N(c2ccc3c(c2)C(C)(C)c2ccccc2-3)c2c([2H])c([2H])c(-c3cccc(-c4ccc5ccccc5c4)c3)c([2H])c2[2H])c([2H])c([2H])c1-c1ccc(-c2ccccc2)cc1]

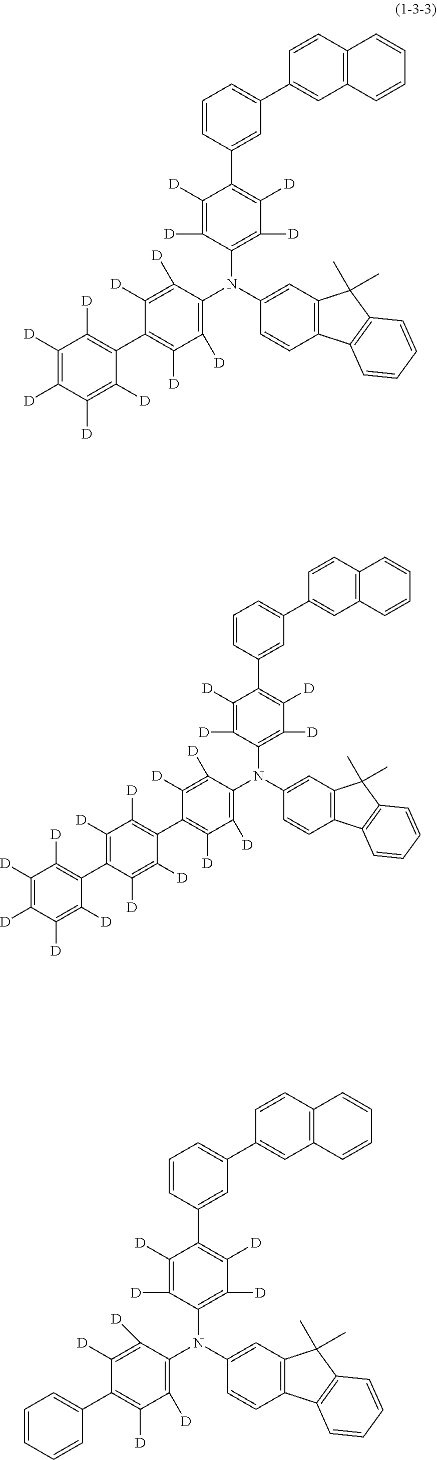 [SMILES: [2H]c1c([2H])c([2H])c(-c2c([2H])c([2H])c(N(c3ccc4c(c3)C(C)(C)c3ccccc3-4)c3c([2H])c([2H])c(-c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc5c(c4)C(C)(C)c4ccccc4-5)c4c([2H])c([2H])c(-c5cccc(-c6ccc7ccccc7c6)c5)c([2H])c4[2H])c([2H])c3[2H])c([2H])c2[2H])c([2H])c1[2H] ; [2H]c1c([2H])c(N(c2ccc3c(c2)C(C)(C)c2ccccc2-3)c2c([2H])c([2H])c(-c3cccc(-c4ccc5ccccc5c4)c3)c([2H])c2[2H])c([2H])c([2H])c1-c1ccccc1]

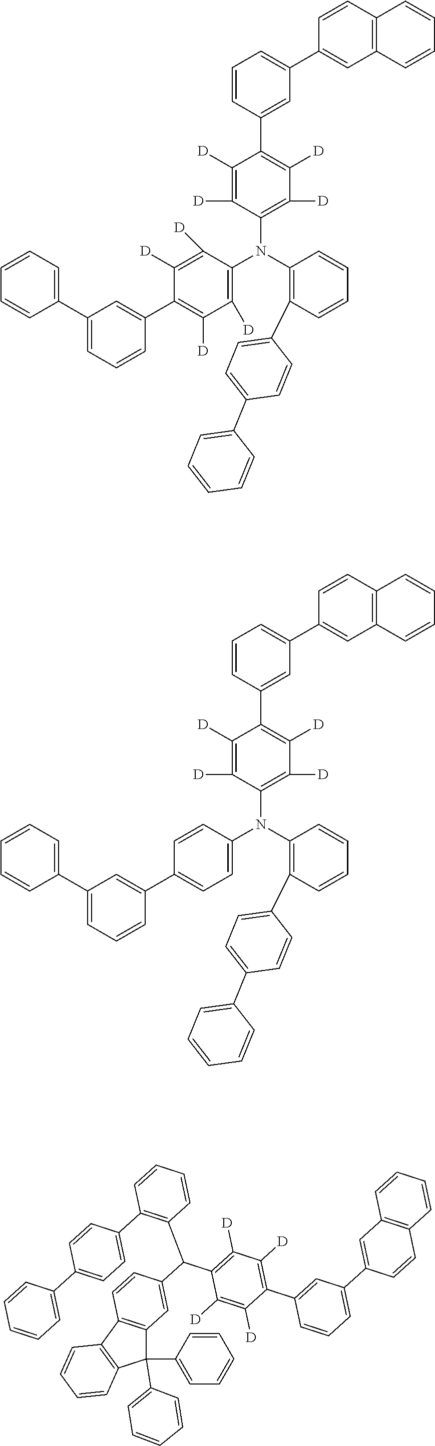 [SMILES: [2H]c1c([2H])c(N(c2ccccc2-c2ccc(-c3ccccc3)cc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2ccccc2-c2ccc(-c3ccccc3)cc2)c([2H])c([2H])c1-c1cccc(-c2ccc3ccccc3c2)c1 ; [2H]c1c([2H])c(C(c2ccc3c(c2)C(c2ccccc2)(c2ccccc2)c2ccccc2-3)c2ccccc2-c2ccc(-c3ccccc3)cc2)c([2H])c([2H])c1-c1cccc(-c2ccc3ccccc3c2)c1]

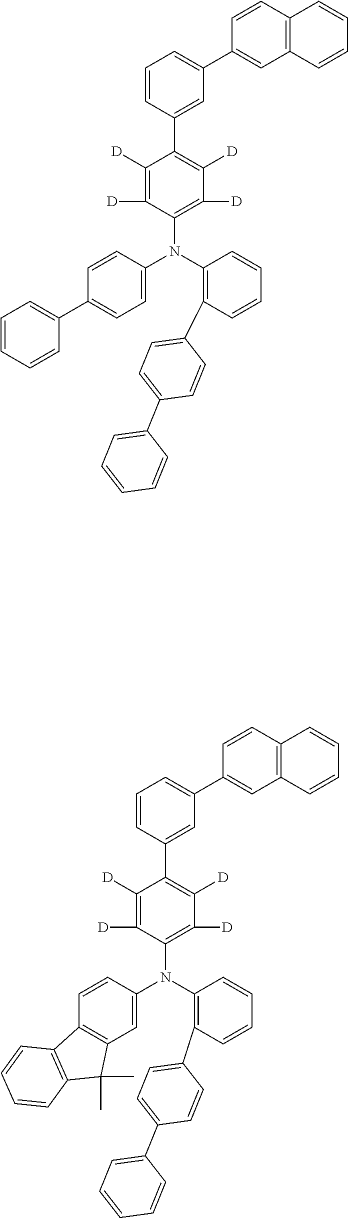 [SMILES: [2H]c1c([2H])c(N(c2ccc(-c3ccccc3)cc2)c2ccccc2-c2ccc(-c3ccccc3)cc2)c([2H])c([2H])c1-c1cccc(-c2ccc3ccccc3c2)c1 ; [2H]c1c([2H])c(N(c2ccc3c(c2)C(C)(C)c2ccccc2-3)c2ccccc2-c2ccc(-c3ccccc3)cc2)c([2H])c([2H])c1-c1cccc(-c2ccc3ccccc3c2)c1]

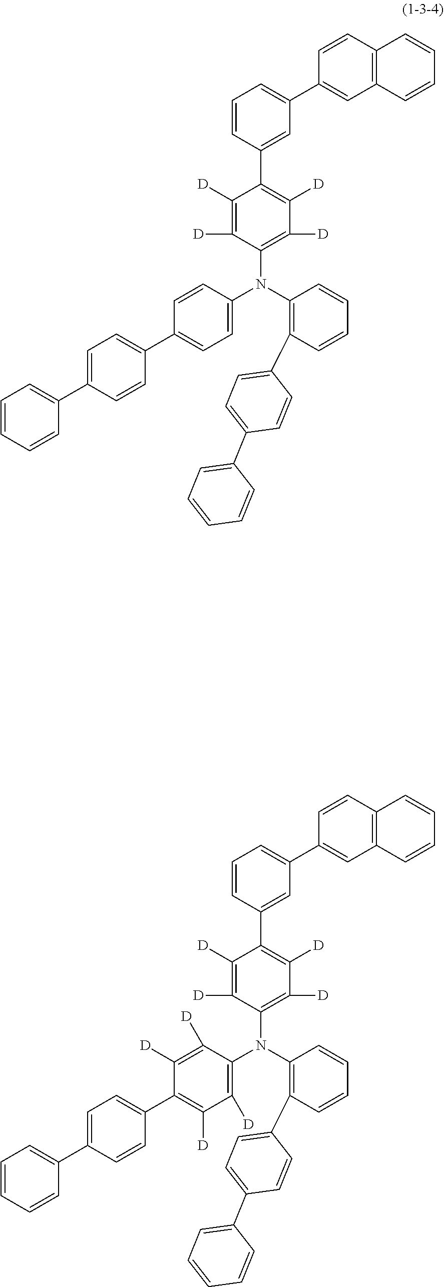 [SMILES: [2H]c1c([2H])c(N(c2ccc(-c3ccc(-c4ccccc4)cc3)cc2)c2ccccc2-c2ccc(-c3ccccc3)cc2)c([2H])c([2H])c1-c1cccc(-c2ccc3ccccc3c2)c1 ; [2H]c1c([2H])c(N(c2ccccc2-c2ccc(-c3ccccc3)cc2)c2c([2H])c([2H])c(-c3cccc(-c4ccc5ccccc5c4)c3)c([2H])c2[2H])c([2H])c([2H])c1-c1ccc(-c2ccccc2)cc1]

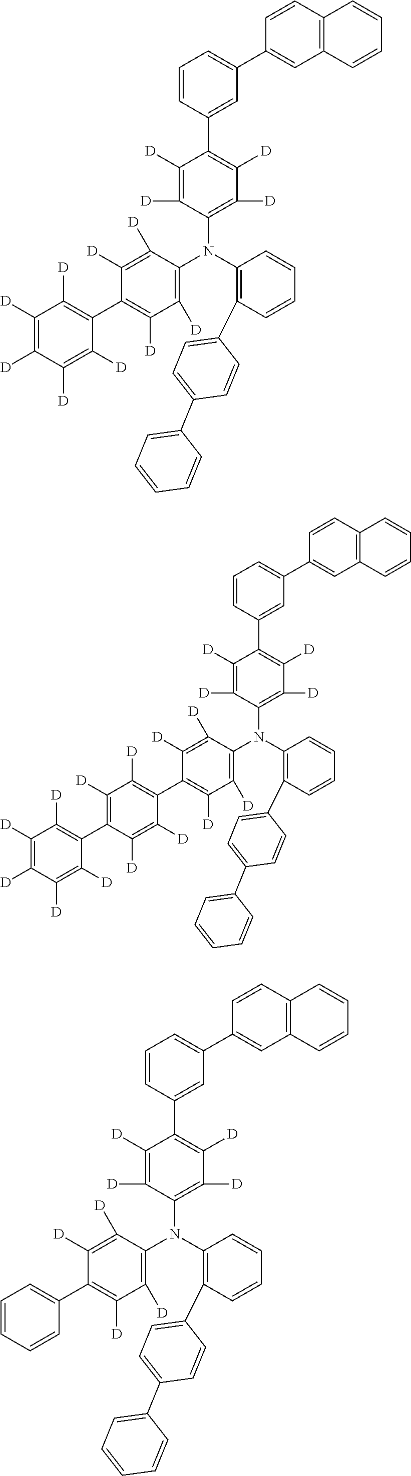

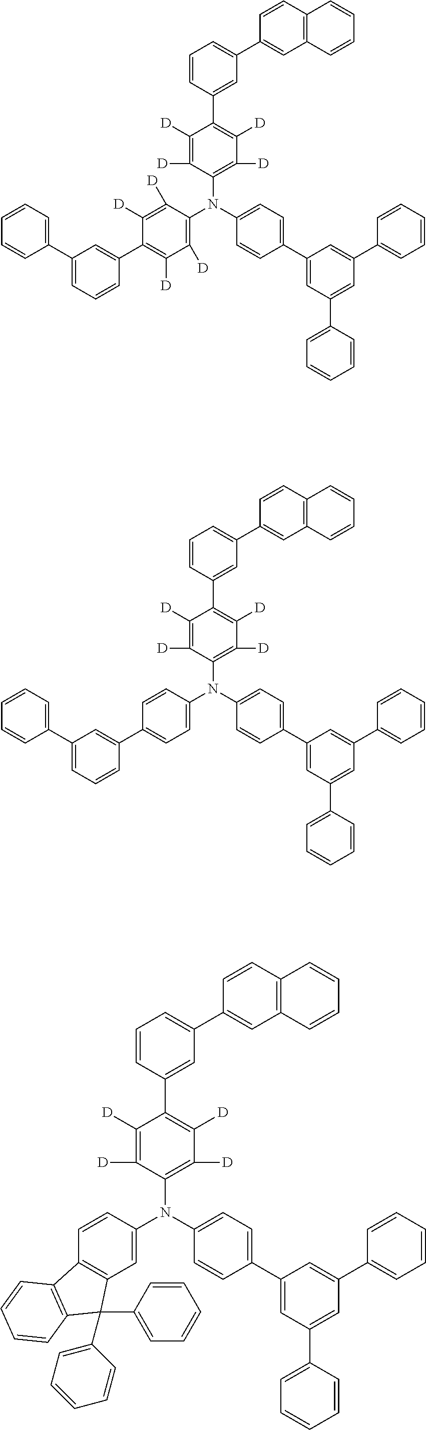 [SMILES: [2H]c1c([2H])c(N(c2ccc(-c3cc(-c4ccccc4)cc(-c4ccccc4)c3)cc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2ccc(-c3cc(-c4ccccc4)cc(-c4ccccc4)c3)cc2)c([2H])c([2H])c1-c1cccc(-c2ccc3ccccc3c2)c1 ; [2H]c1c([2H])c(N(c2ccc(-c3cc(-c4ccccc4)cc(-c4ccccc4)c3)cc2)c2ccc3c(c2)C(c2ccccc2)(c2ccccc2)c2ccccc2-3)c([2H])c([2H])c1-c1cccc(-c2ccc3ccccc3c2)c1]

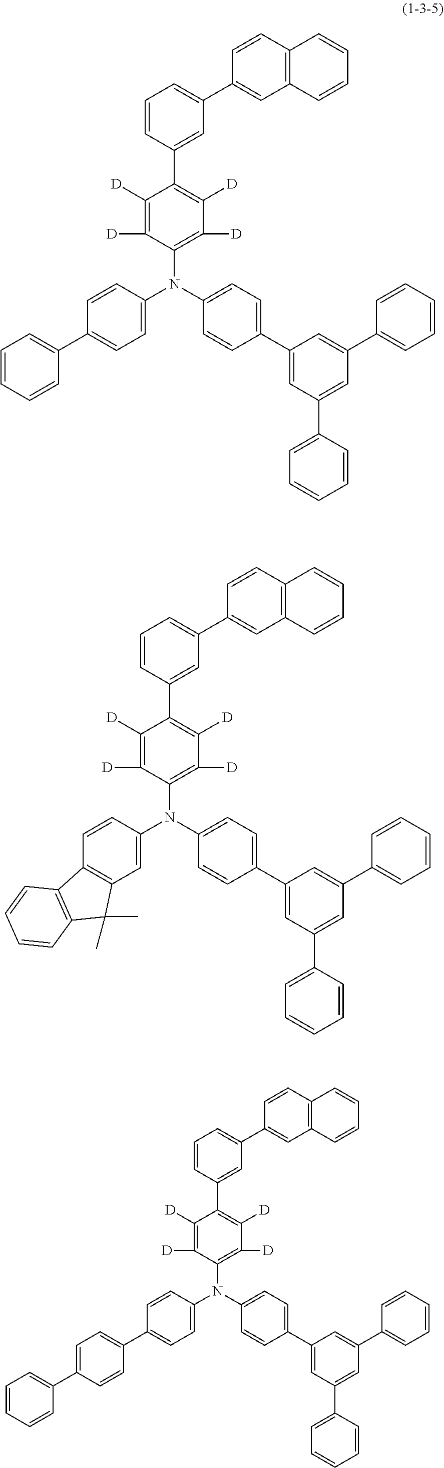 [SMILES: [2H]c1c([2H])c(N(c2ccc(-c3ccccc3)cc2)c2ccc(-c3cc(-c4ccccc4)cc(-c4ccccc4)c3)cc2)c([2H])c([2H])c1-c1cccc(-c2ccc3ccccc3c2)c1 ; [2H]c1c([2H])c(N(c2ccc(-c3cc(-c4ccccc4)cc(-c4ccccc4)c3)cc2)c2ccc3c(c2)C(C)(C)c2ccccc2-3)c([2H])c([2H])c1-c1cccc(-c2ccc3ccccc3c2)c1 ; [2H]c1c([2H])c(N(c2ccc(-c3ccc(-c4ccccc4)cc3)cc2)c2ccc(-c3cc(-c4ccccc4)cc(-c4ccccc4)c3)cc2)c([2H])c([2H])c1-c1cccc(-c2ccc3ccccc3c2)c1]

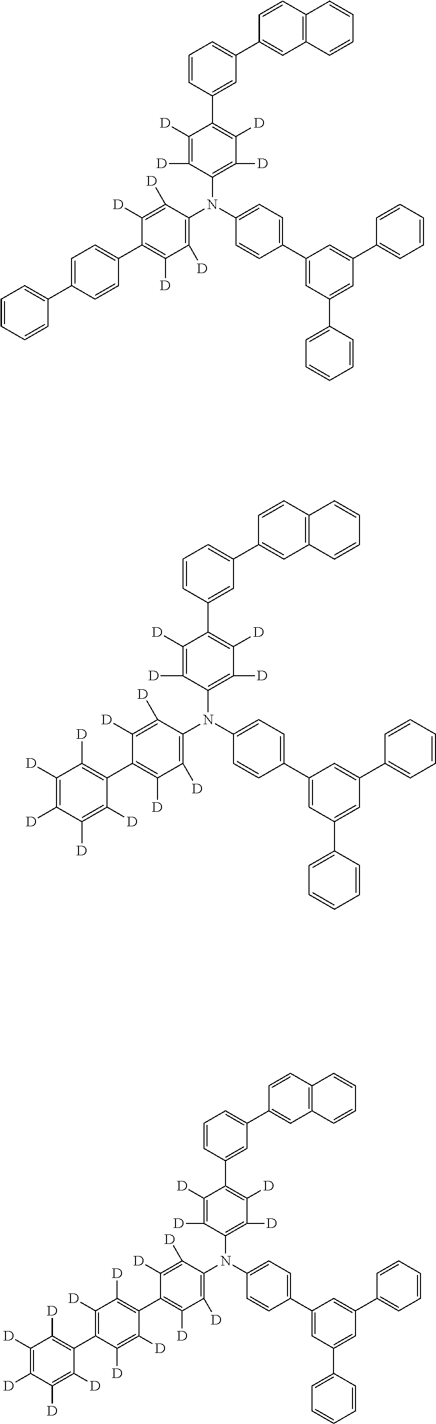

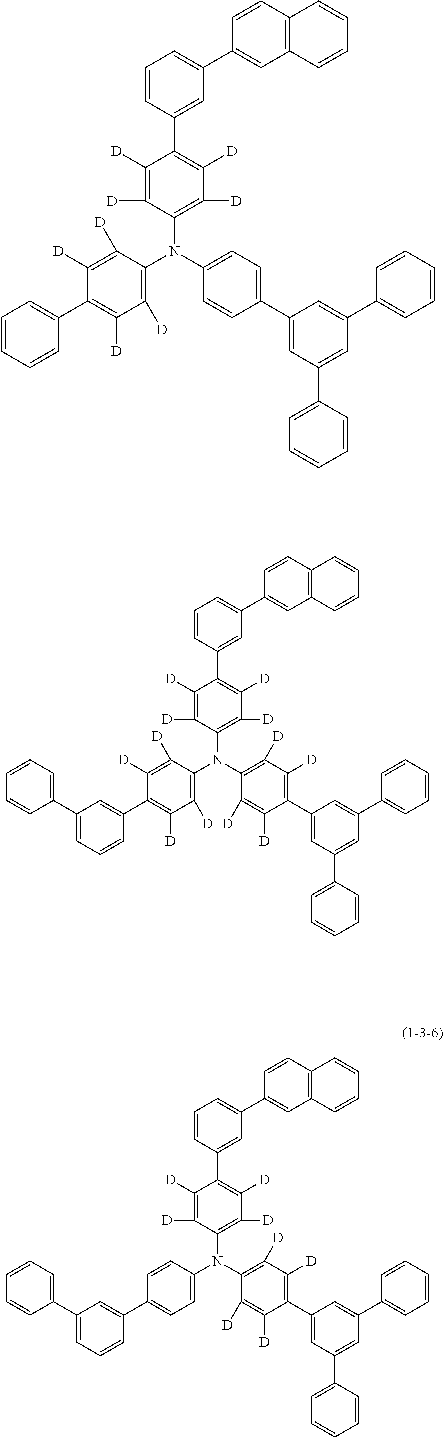 [SMILES: [2H]c1c([2H])c(N(c2ccc(-c3cc(-c4ccccc4)cc(-c4ccccc4)c3)cc2)c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2c([2H])c([2H])c(-c3cccc(-c4ccc5ccccc5c4)c3)c([2H])c2[2H])c2c([2H])c([2H])c(-c3cc(-c4ccccc4)cc(-c4ccccc4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2c([2H])c([2H])c(-c3cc(-c4ccccc4)cc(-c4ccccc4)c3)c([2H])c2[2H])c([2H])c([2H])c1-c1cccc(-c2ccc3ccccc3c2)c1]

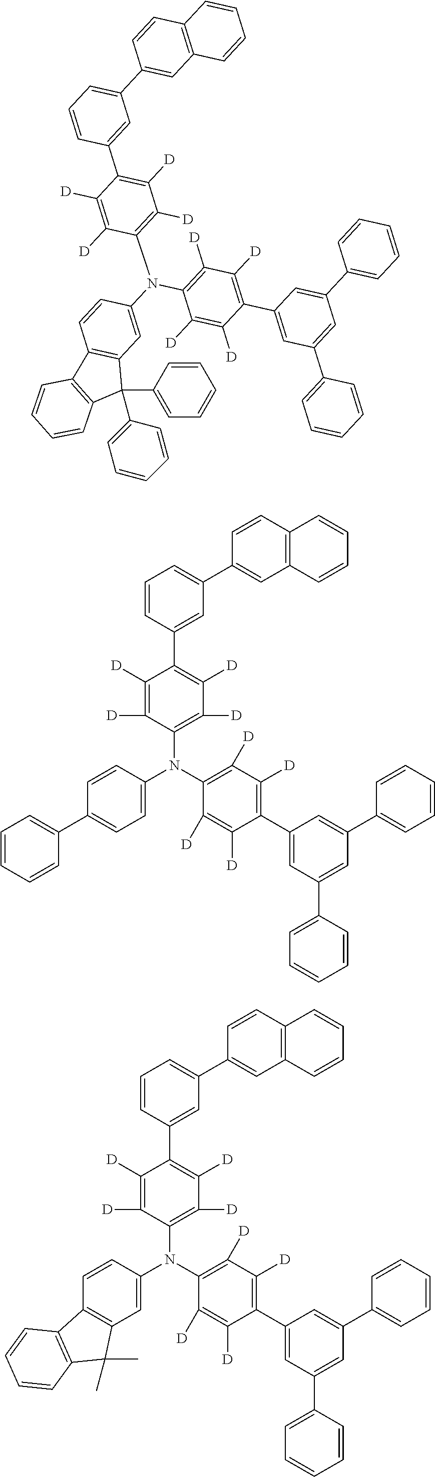

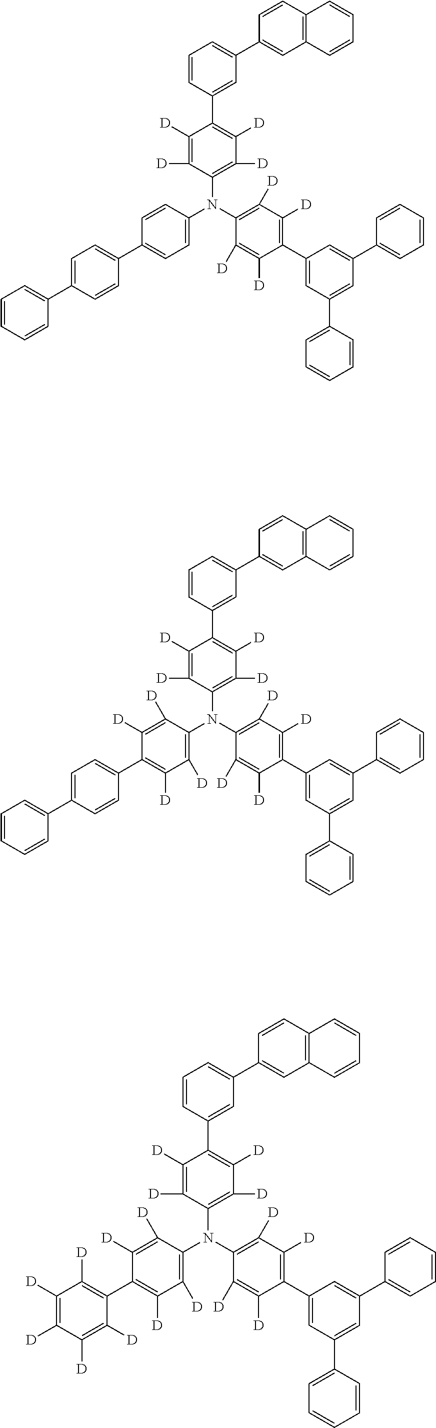

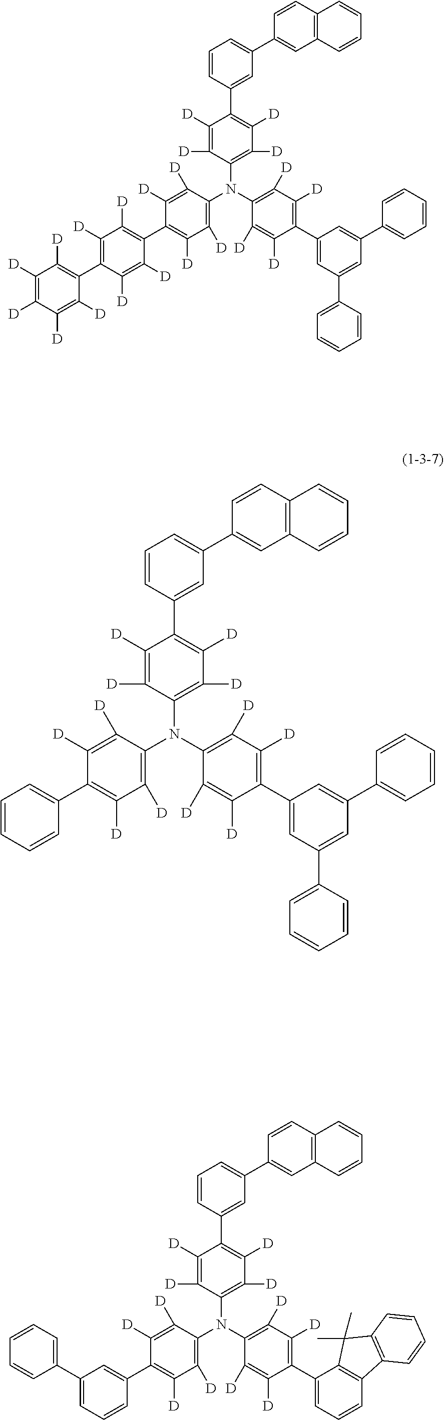

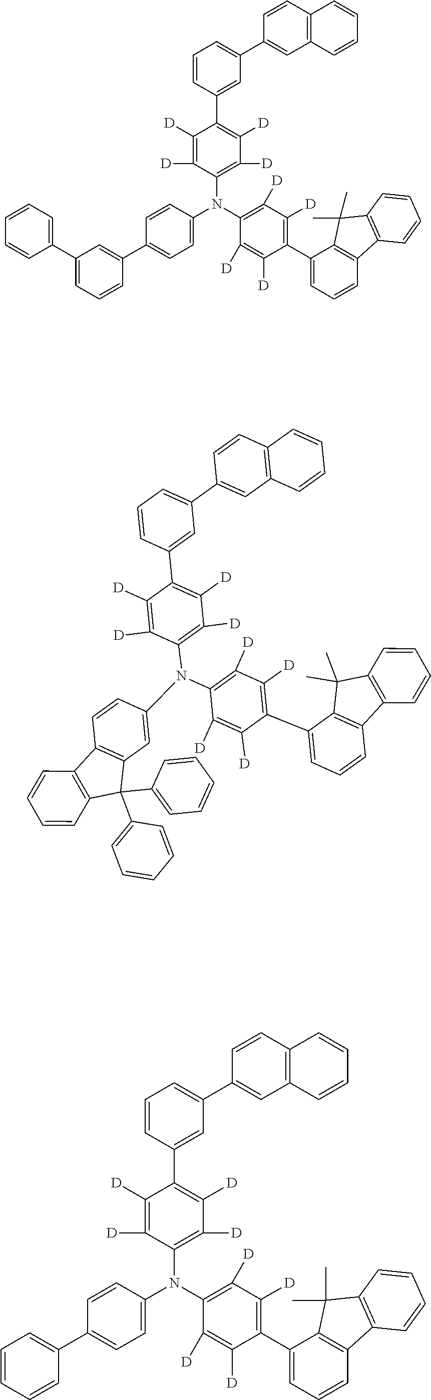 [SMILES: [2H]c1c([2H])c(N(c2ccc(-c3cccc(-c4ccccc4)c3)cc2)c2c([2H])c([2H])c(-c3cccc4c3C(C)(C)c3ccccc3-4)c([2H])c2[2H])c([2H])c([2H])c1-c1cccc(-c2ccc3ccccc3c2)c1 ; [2H]c1c([2H])c(N(c2ccc3c(c2)C(c2ccccc2)(c2ccccc2)c2ccccc2-3)c2c([2H])c([2H])c(-c3cccc4c3C(C)(C)c3ccccc3-4)c([2H])c2[2H])c([2H])c([2H])c1-c1cccc(-c2ccc3ccccc3c2)c1 ; [2H]c1c([2H])c(N(c2ccc(-c3ccccc3)cc2)c2c([2H])c([2H])c(-c3cccc4c3C(C)(C)c3ccccc3-4)c([2H])c2[2H])c([2H])c([2H])c1-c1cccc(-c2ccc3ccccc3c2)c1]

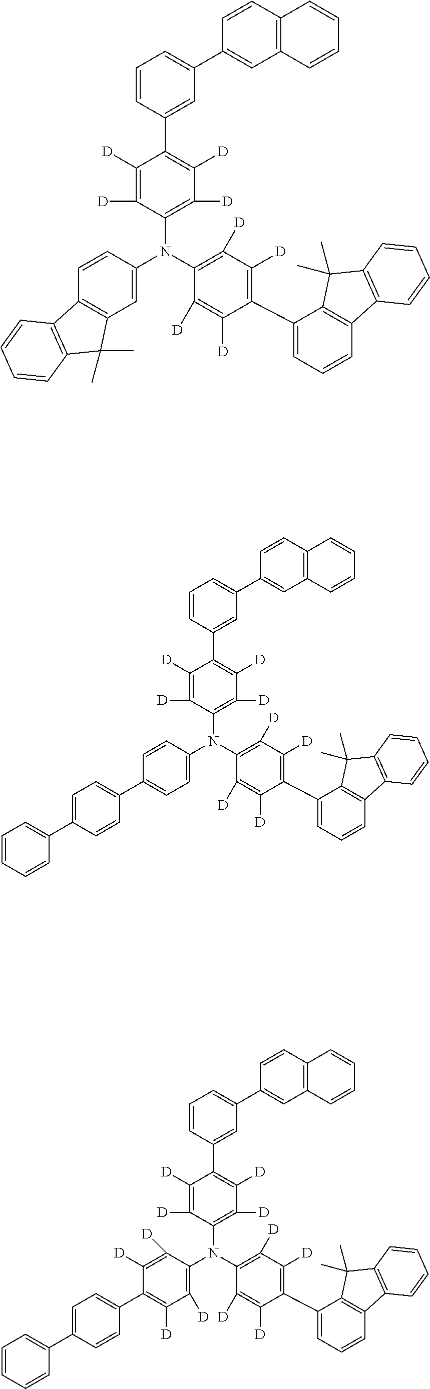 [SMILES: [2H]c1c([2H])c(N(c2ccc3c(c2)C(C)(C)c2ccccc2-3)c2c([2H])c([2H])c(-c3cccc4c3C(C)(C)c3ccccc3-4)c([2H])c2[2H])c([2H])c([2H])c1-c1cccc(-c2ccc3ccccc3c2)c1 ; [2H]c1c([2H])c(N(c2ccc(-c3ccc(-c4ccccc4)cc3)cc2)c2c([2H])c([2H])c(-c3cccc4c3C(C)(C)c3ccccc3-4)c([2H])c2[2H])c([2H])c([2H])c1-c1cccc(-c2ccc3ccccc3c2)c1 ; [2H]c1c([2H])c(N(c2c([2H])c([2H])c(-c3cccc(-c4ccc5ccccc5c4)c3)c([2H])c2[2H])c2c([2H])c([2H])c(-c3cccc4c3C(C)(C)c3ccccc3-4)c([2H])c2[2H])c([2H])c([2H])c1-c1ccc(-c2ccccc2)cc1]

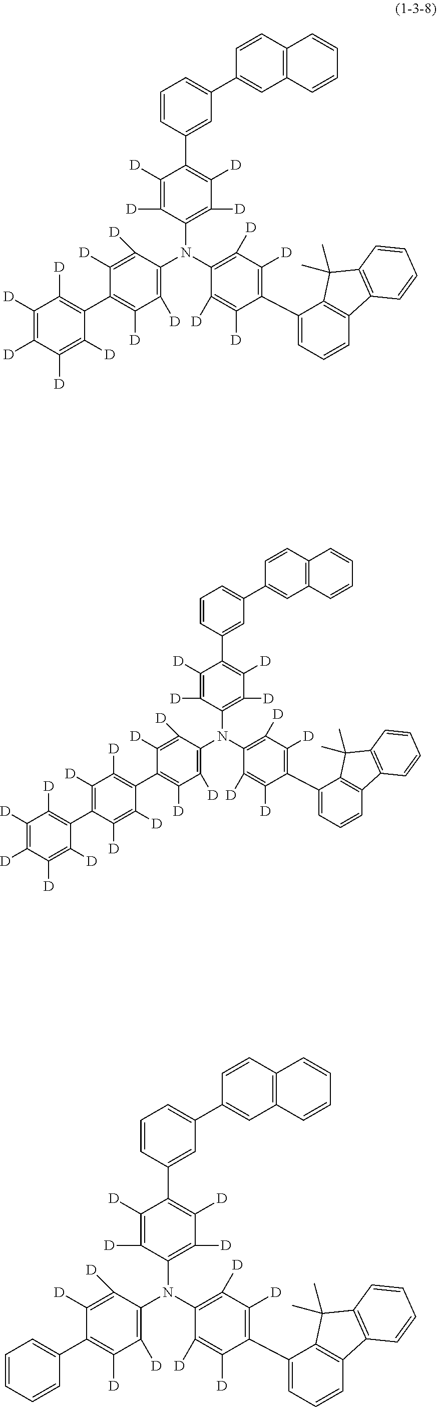

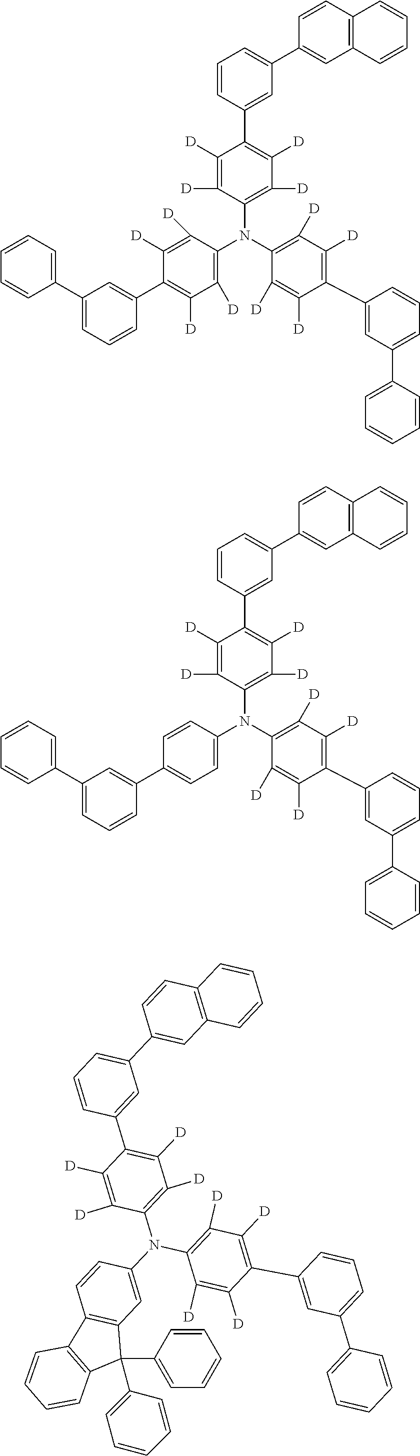 [SMILES: [2H]c1c([2H])c(N(c2c([2H])c([2H])c(-c3cccc(-c4ccccc4)c3)c([2H])c2[2H])c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc3c(c2)C(c2ccccc2)(c2ccccc2)c2ccccc2-3)c2c([2H])c([2H])c(-c3cccc(-c4ccc5ccccc5c4)c3)c([2H])c2[2H])c([2H])c([2H])c1-c1cccc(-c2ccccc2)c1]

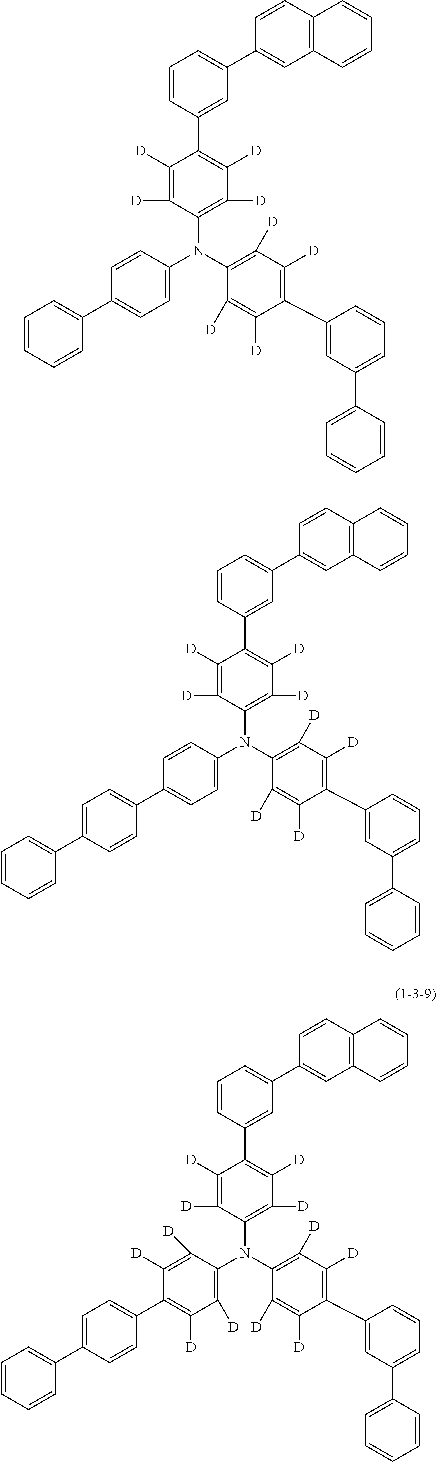

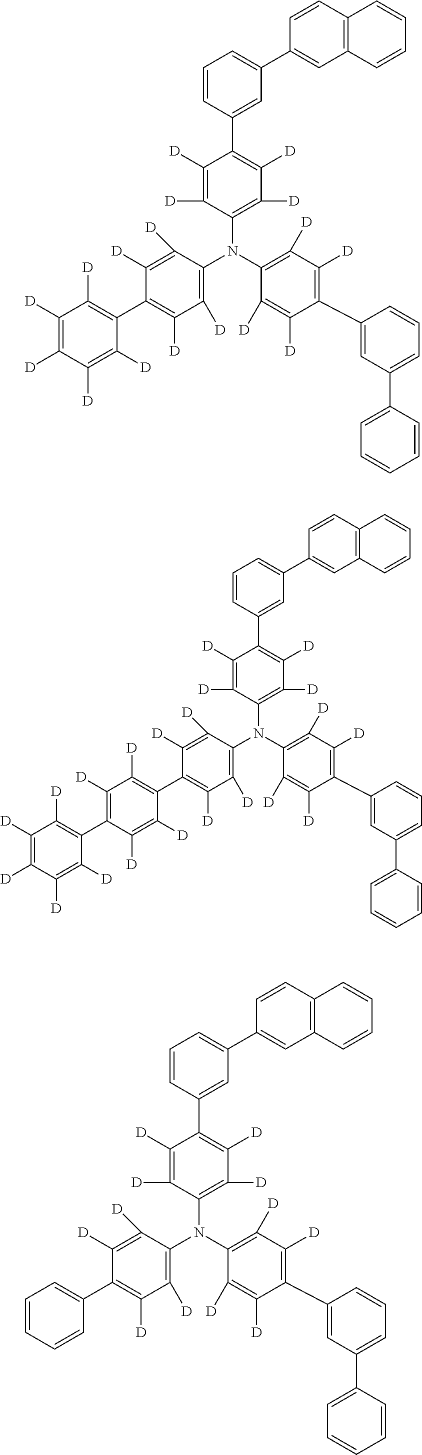

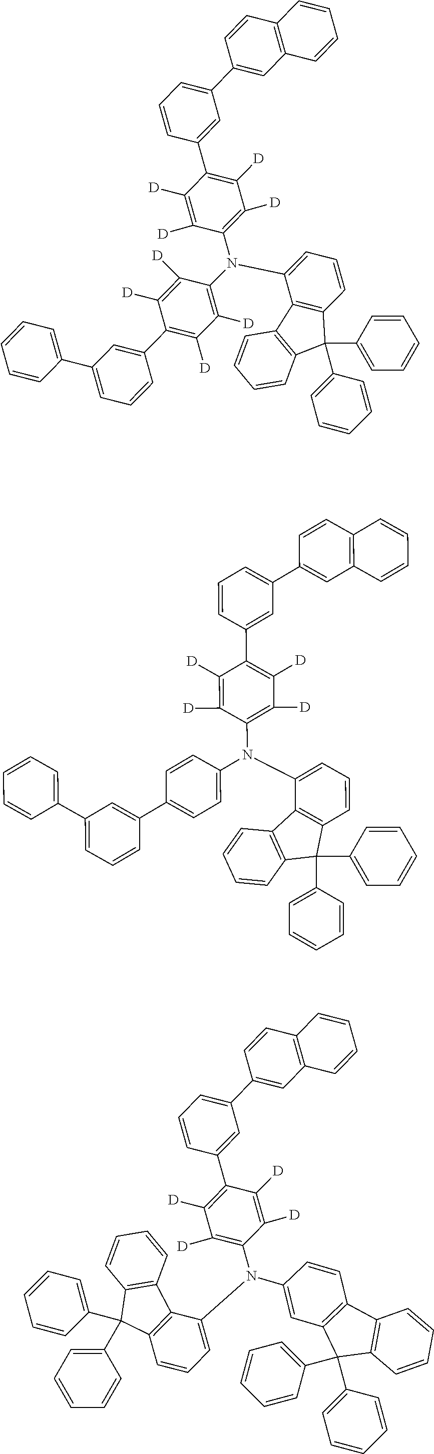 [SMILES: [2H]c1c([2H])c(N(c2cccc3c2-c2ccccc2C3(c2ccccc2)c2ccccc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2cccc3c2-c2ccccc2C3(c2ccccc2)c2ccccc2)c([2H])c([2H])c1-c1cccc(-c2ccc3ccccc3c2)c1 ; [2H]c1c([2H])c(N(c2ccc3c(c2)C(c2ccccc2)(c2ccccc2)c2ccccc2-3)c2cccc3c2-c2ccccc2C3(c2ccccc2)c2ccccc2)c([2H])c([2H])c1-c1cccc(-c2ccc3ccccc3c2)c1]

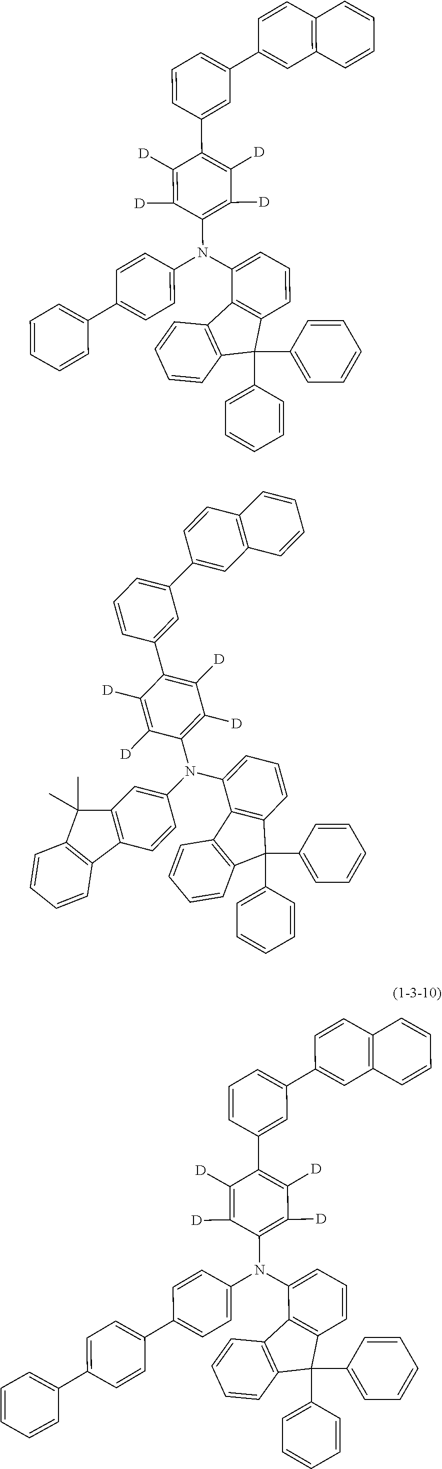

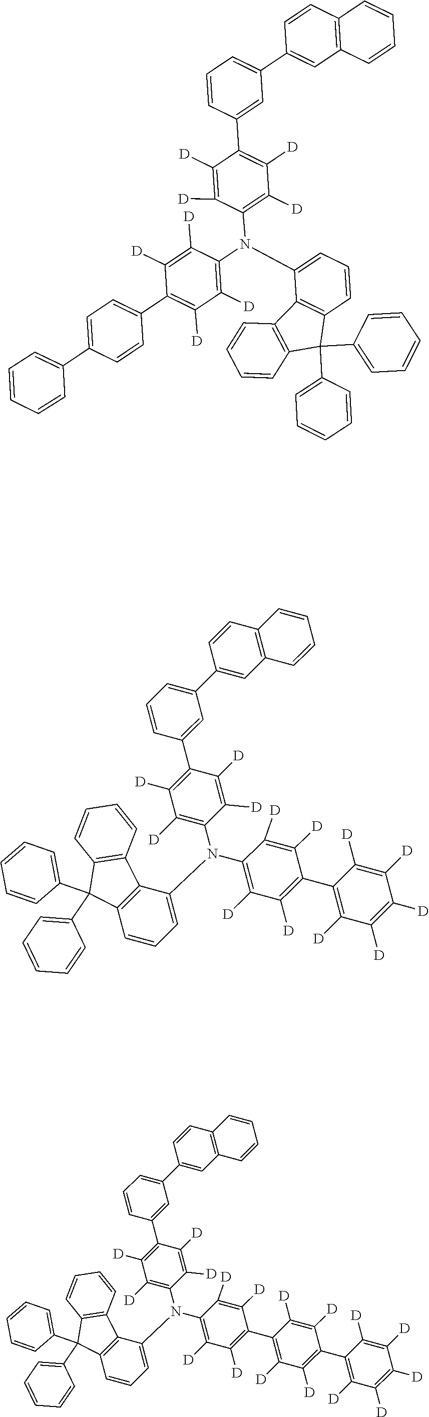

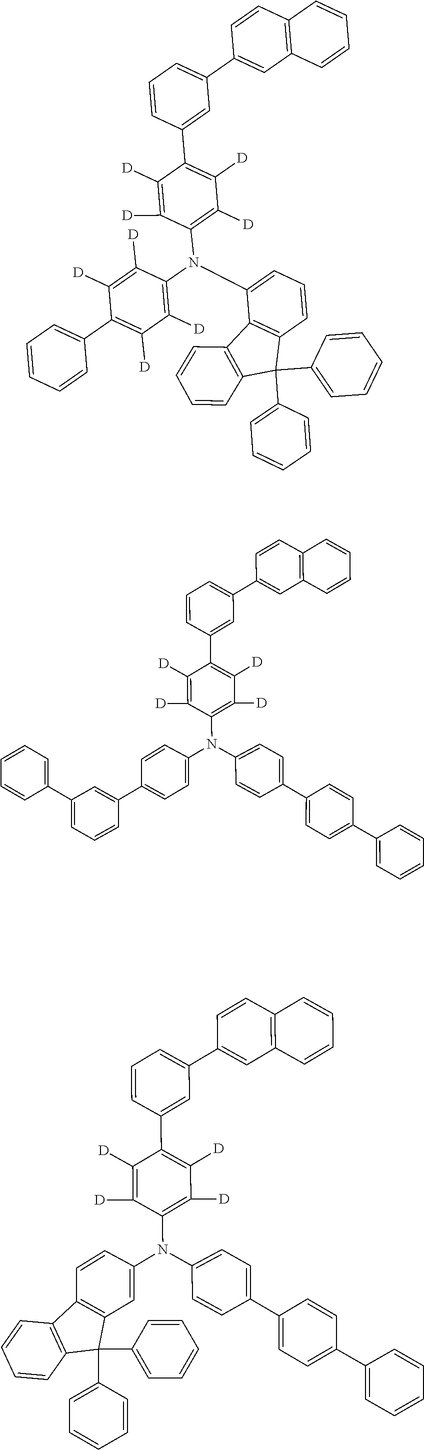 [SMILES: [2H]c1c([2H])c(N(c2cccc3c2-c2ccccc2C3(c2ccccc2)c2ccccc2)c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2ccc(-c3ccc(-c4ccccc4)cc3)cc2)c2ccc(-c3cccc(-c4ccccc4)c3)cc2)c([2H])c([2H])c1-c1cccc(-c2ccc3ccccc3c2)c1 ; [2H]c1c([2H])c(N(c2ccc(-c3ccc(-c4ccccc4)cc3)cc2)c2ccc3c(c2)C(c2ccccc2)(c2ccccc2)c2ccccc2-3)c([2H])c([2H])c1-c1cccc(-c2ccc3ccccc3c2)c1]

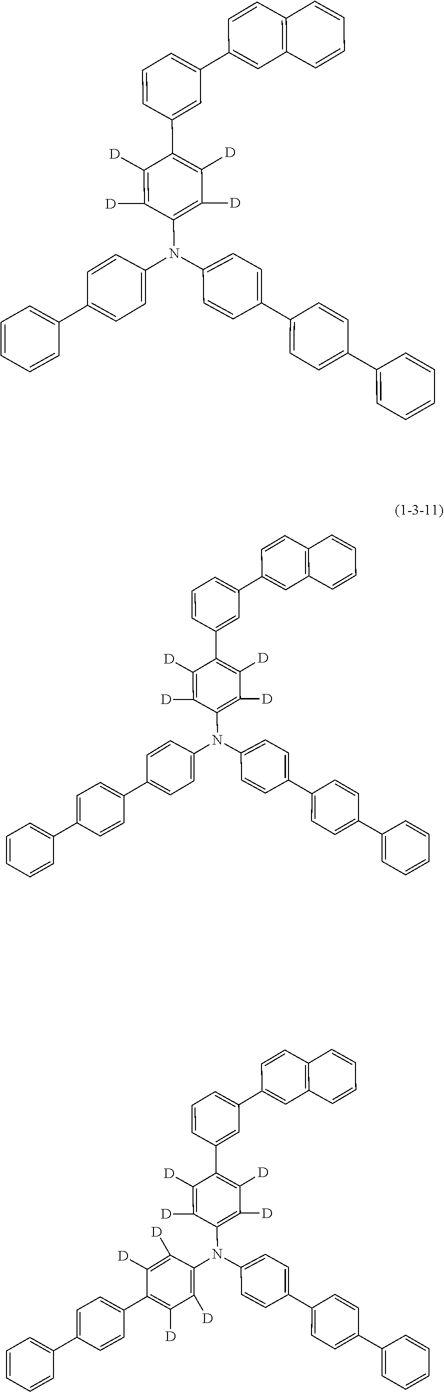 [SMILES: [2H]c1c([2H])c(N(c2ccc(-c3ccccc3)cc2)c2ccc(-c3ccc(-c4ccccc4)cc3)cc2)c([2H])c([2H])c1-c1cccc(-c2ccc3ccccc3c2)c1 ; [2H]c1c([2H])c(N(c2ccc(-c3ccc(-c4ccccc4)cc3)cc2)c2ccc(-c3ccc(-c4ccccc4)cc3)cc2)c([2H])c([2H])c1-c1cccc(-c2ccc3ccccc3c2)c1 ; [2H]c1c([2H])c(N(c2ccc(-c3ccc(-c4ccccc4)cc3)cc2)c2c([2H])c([2H])c(-c3cccc(-c4ccc5ccccc5c4)c3)c([2H])c2[2H])c([2H])c([2H])c1-c1ccc(-c2ccccc2)cc1]

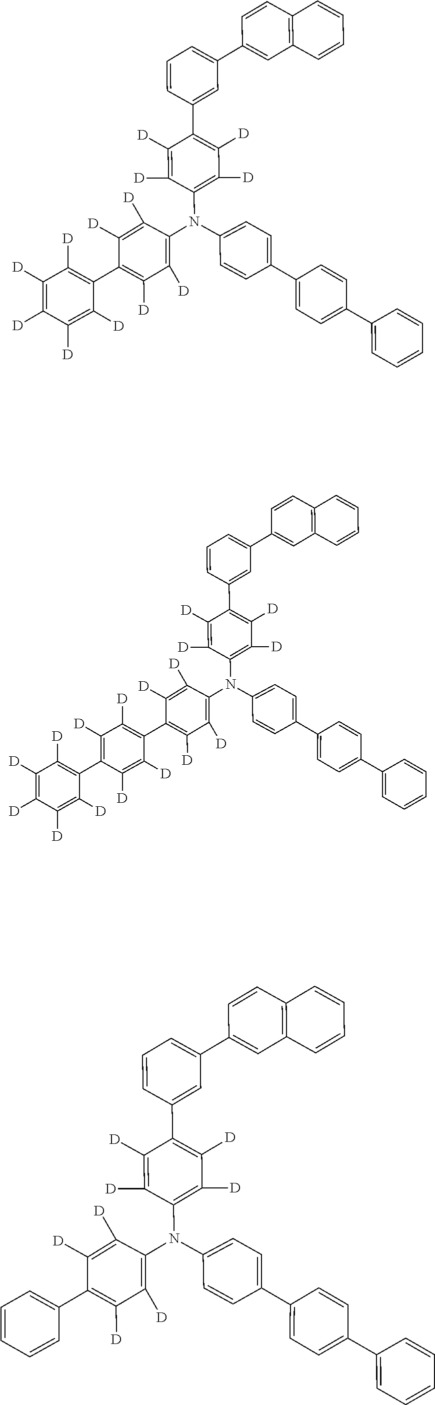 [SMILES: [2H]c1c([2H])c([2H])c(-c2c([2H])c([2H])c(N(c3ccc(-c4ccc(-c5ccccc5)cc4)cc3)c3c([2H])c([2H])c(-c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5ccc(-c6ccccc6)cc5)cc4)c4c([2H])c([2H])c(-c5cccc(-c6ccc7ccccc7c6)c5)c([2H])c4[2H])c([2H])c3[2H])c([2H])c2[2H])c([2H])c1[2H] ; [2H]c1c([2H])c(N(c2ccc(-c3ccc(-c4ccccc4)cc3)cc2)c2c([2H])c([2H])c(-c3cccc(-c4ccc5ccccc5c4)c3)c([2H])c2[2H])c([2H])c([2H])c1-c1ccccc1]

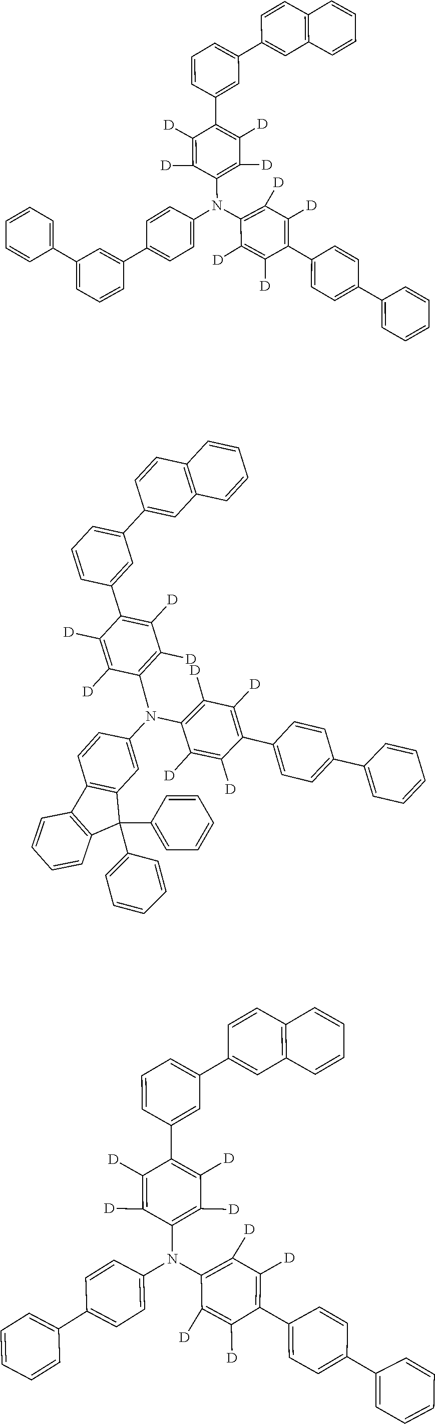 [SMILES: [2H]c1c([2H])c(N(c2ccc(-c3cccc(-c4ccccc4)c3)cc2)c2c([2H])c([2H])c(-c3cccc(-c4ccc5ccccc5c4)c3)c([2H])c2[2H])c([2H])c([2H])c1-c1ccc(-c2ccccc2)cc1 ; [2H]c1c([2H])c(N(c2ccc3c(c2)C(c2ccccc2)(c2ccccc2)c2ccccc2-3)c2c([2H])c([2H])c(-c3cccc(-c4ccc5ccccc5c4)c3)c([2H])c2[2H])c([2H])c([2H])c1-c1ccc(-c2ccccc2)cc1 ; [2H]c1c([2H])c(N(c2ccc(-c3ccccc3)cc2)c2c([2H])c([2H])c(-c3cccc(-c4ccc5ccccc5c4)c3)c([2H])c2[2H])c([2H])c([2H])c1-c1ccc(-c2ccccc2)cc1]

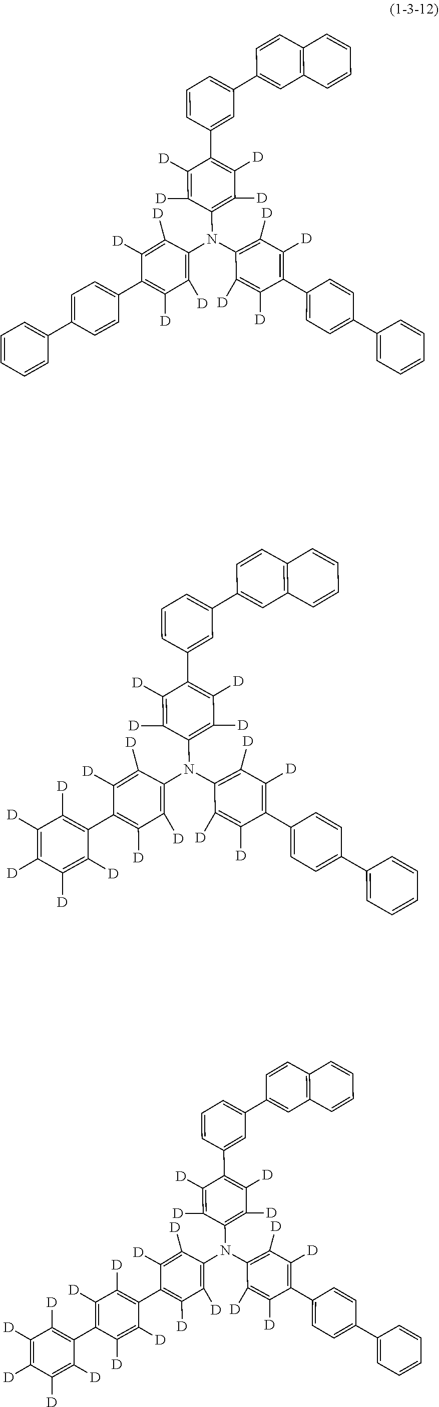

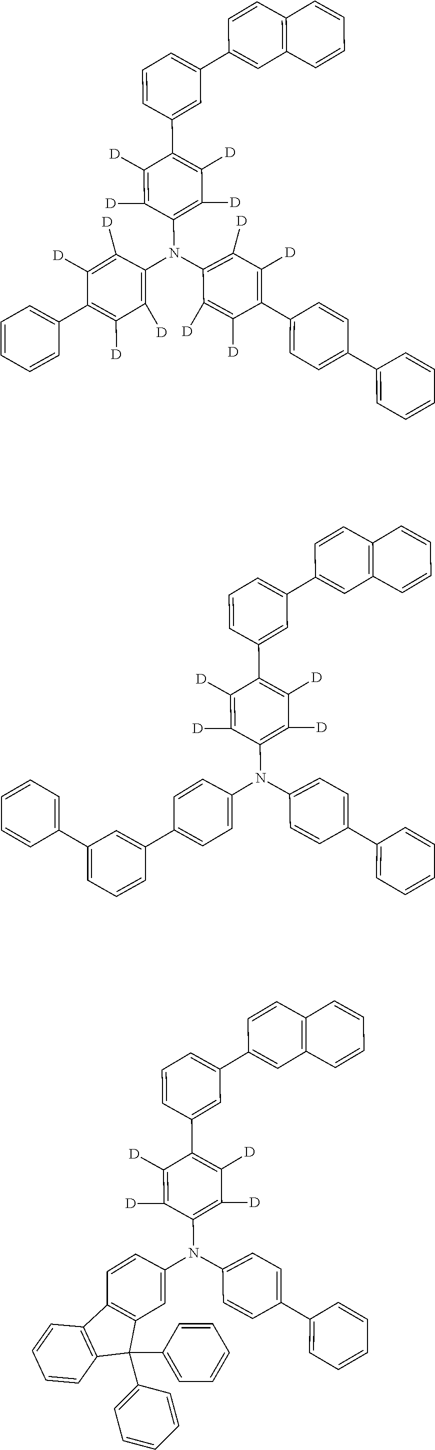 [SMILES: [2H]c1c([2H])c(N(c2c([2H])c([2H])c(-c3ccc(-c4ccccc4)cc3)c([2H])c2[2H])c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2ccc(-c3ccccc3)cc2)c2ccc(-c3cccc(-c4ccccc4)c3)cc2)c([2H])c([2H])c1-c1cccc(-c2ccc3ccccc3c2)c1 ; [2H]c1c([2H])c(N(c2ccc(-c3ccccc3)cc2)c2ccc3c(c2)C(c2ccccc2)(c2ccccc2)c2ccccc2-3)c([2H])c([2H])c1-c1cccc(-c2ccc3ccccc3c2)c1]

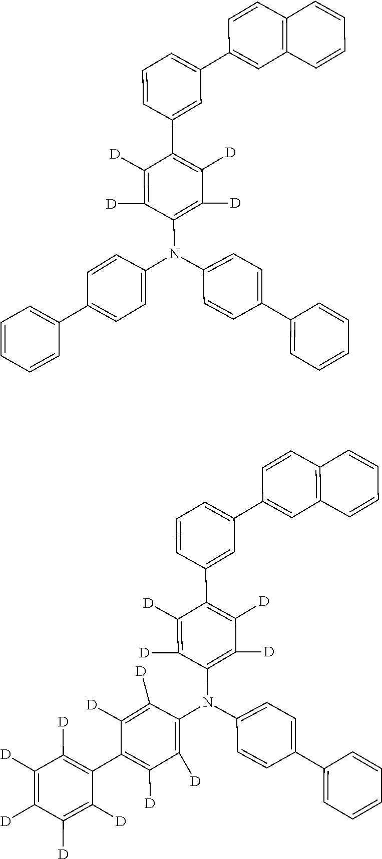 [SMILES: [2H]c1c([2H])c(N(c2ccc(-c3ccccc3)cc2)c2ccc(-c3ccccc3)cc2)c([2H])c([2H])c1-c1cccc(-c2ccc3ccccc3c2)c1 ; [2H]c1c([2H])c([2H])c(-c2c([2H])c([2H])c(N(c3ccc(-c4ccccc4)cc3)c3c([2H])c([2H])c(-c4cccc(-c5ccc6ccccc6c5)c4)c([2H])c3[2H])c([2H])c2[2H])c([2H])c1[2H]]

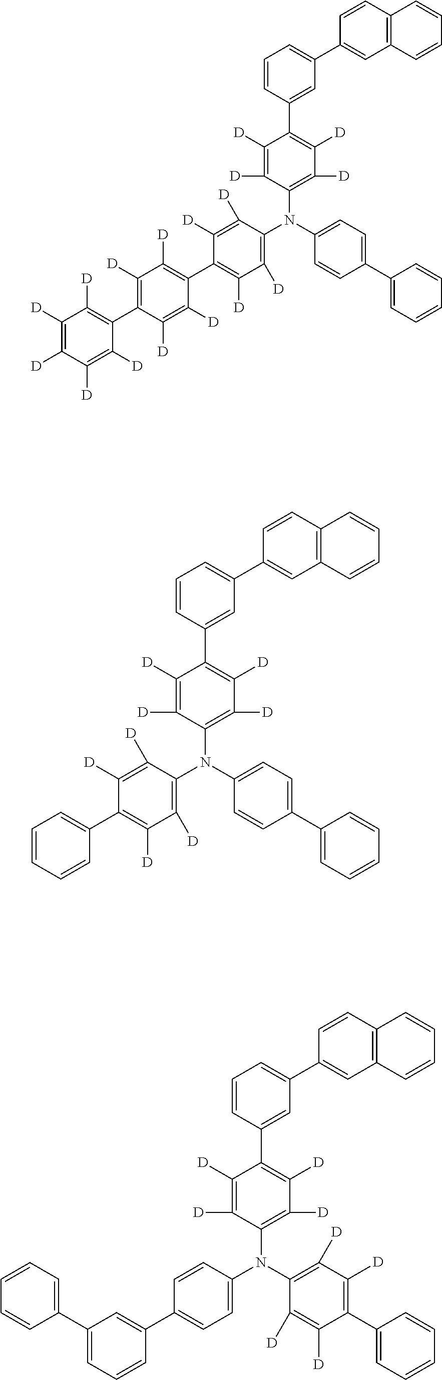 [SMILES: [2H]c1c([2H])c([2H])c(-c2c([2H])c([2H])c(-c3c([2H])c([2H])c(N(c4ccc(-c5ccccc5)cc4)c4c([2H])c([2H])c(-c5cccc(-c6ccc7ccccc7c6)c5)c([2H])c4[2H])c([2H])c3[2H])c([2H])c2[2H])c([2H])c1[2H] ; [2H]c1c([2H])c(N(c2ccc(-c3ccccc3)cc2)c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2ccc(-c3cccc(-c4ccccc4)c3)cc2)c2c([2H])c([2H])c(-c3cccc(-c4ccc5ccccc5c4)c3)c([2H])c2[2H])c([2H])c([2H])c1-c1ccccc1]

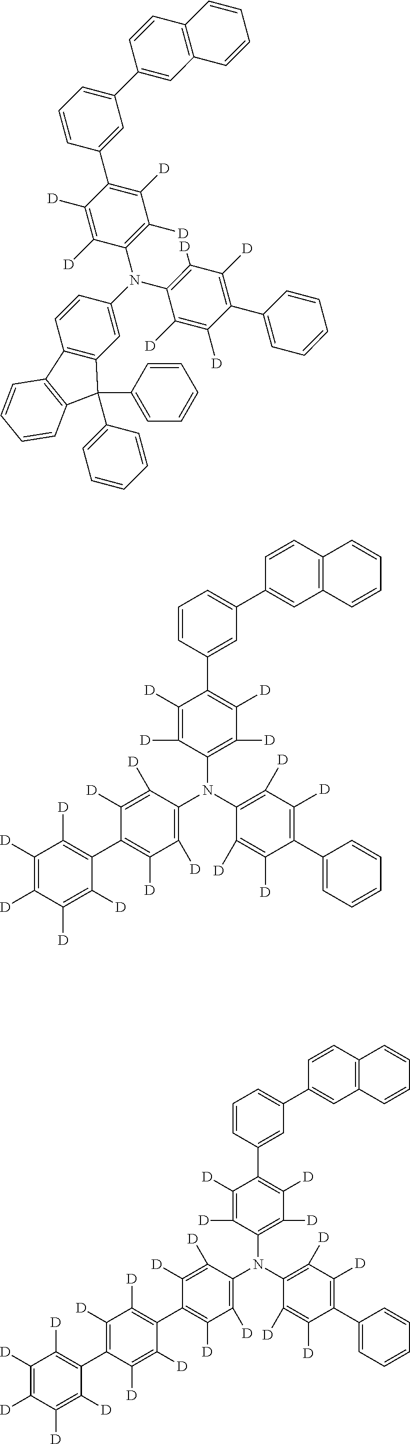

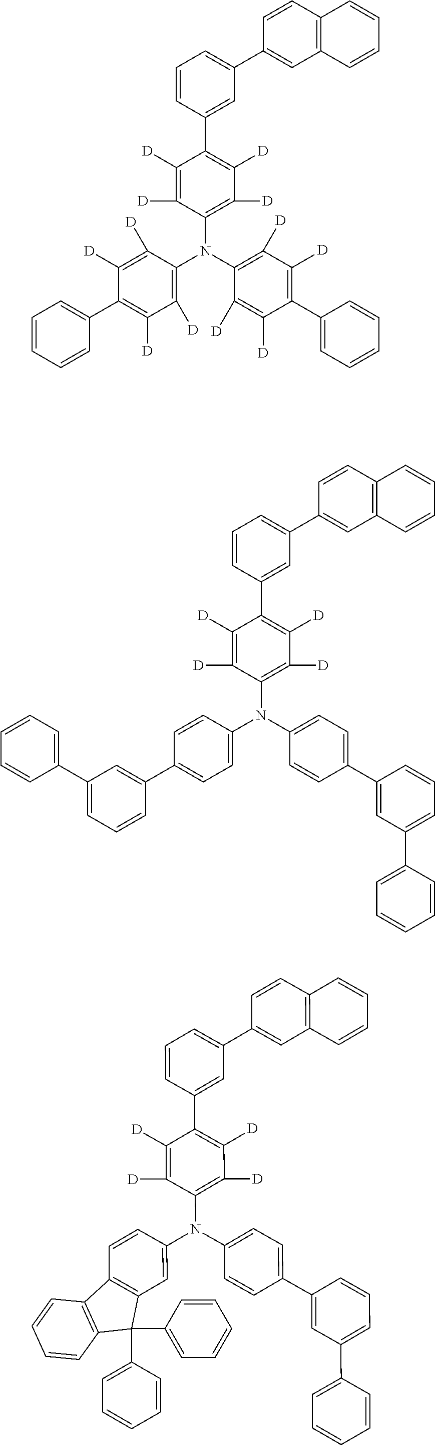 [SMILES: [2H]c1c([2H])c(N(c2c([2H])c([2H])c(-c3ccccc3)c([2H])c2[2H])c2c([2H])c([2H])c(-c3cccc(-c4ccc5ccccc5c4)c3)c([2H])c2[2H])c([2H])c([2H])c1-c1ccccc1 ; [2H]c1c([2H])c(N(c2ccc(-c3cccc(-c4ccccc4)c3)cc2)c2ccc(-c3cccc(-c4ccccc4)c3)cc2)c([2H])c([2H])c1-c1cccc(-c2ccc3ccccc3c2)c1 ; [2H]c1c([2H])c(N(c2ccc(-c3cccc(-c4ccccc4)c3)cc2)c2ccc3c(c2)C(c2ccccc2)(c2ccccc2)c2ccccc2-3)c([2H])c([2H])c1-c1cccc(-c2ccc3ccccc3c2)c1]

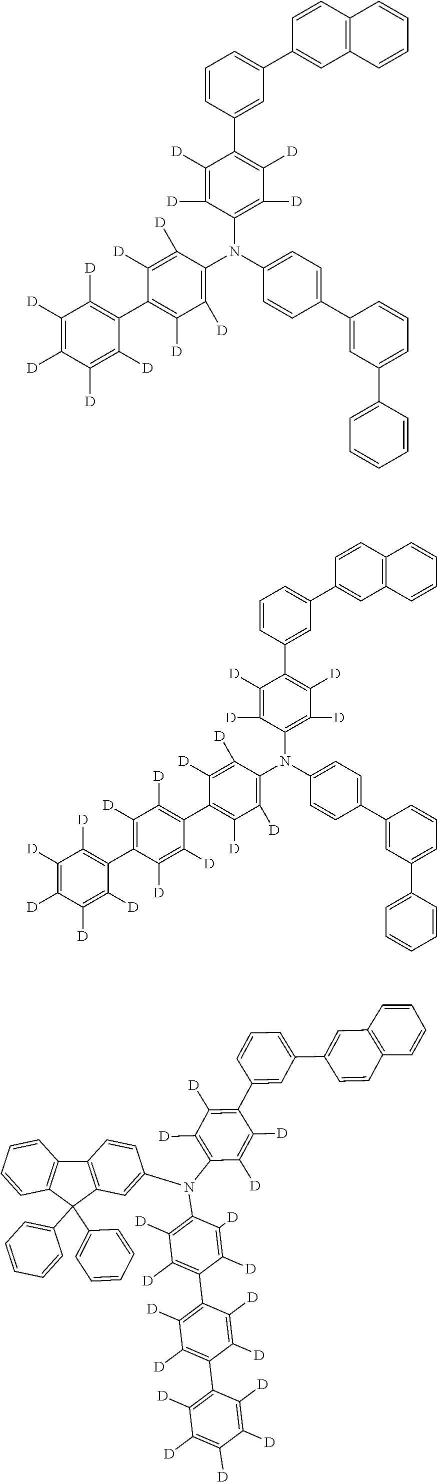

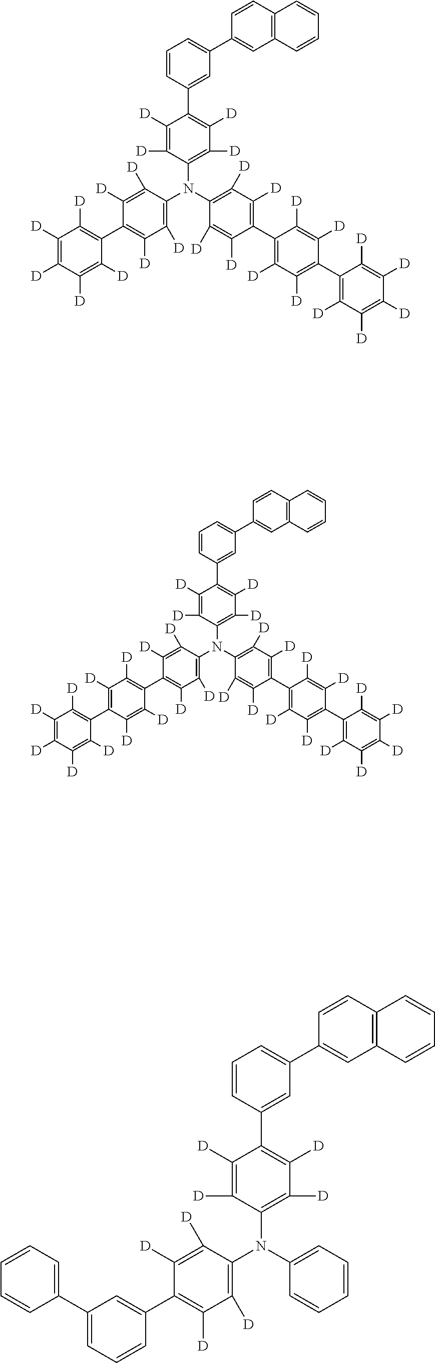

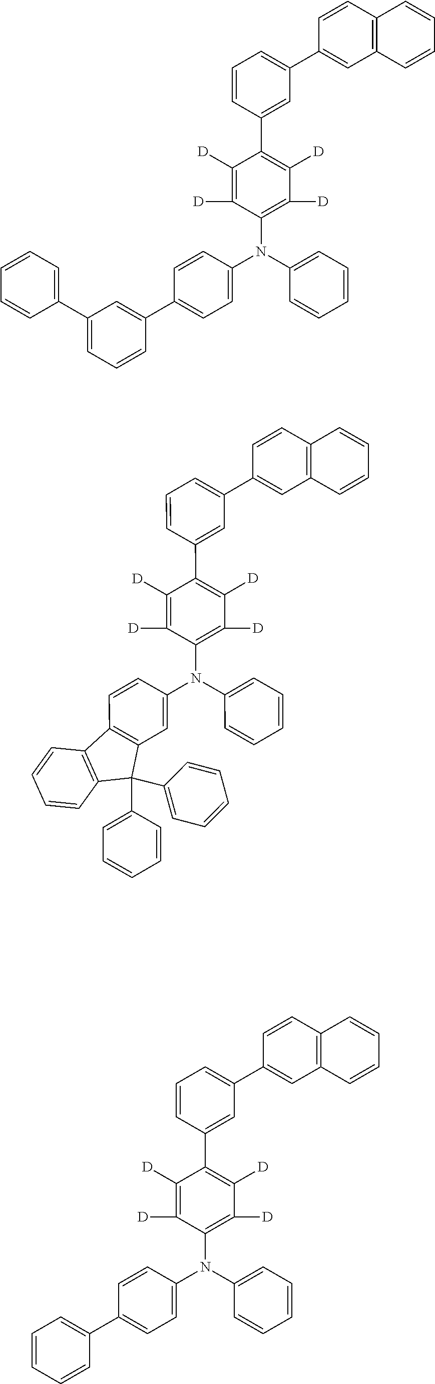 [SMILES: [2H]c1c([2H])c(N(c2ccccc2)c2ccc(-c3cccc(-c4ccccc4)c3)cc2)c([2H])c([2H])c1-c1cccc(-c2ccc3ccccc3c2)c1 ; [2H]c1c([2H])c(N(c2ccccc2)c2ccc3c(c2)C(c2ccccc2)(c2ccccc2)c2ccccc2-3)c([2H])c([2H])c1-c1cccc(-c2ccc3ccccc3c2)c1 ; [2H]c1c([2H])c(N(c2ccccc2)c2ccc(-c3ccccc3)cc2)c([2H])c([2H])c1-c1cccc(-c2ccc3ccccc3c2)c1]

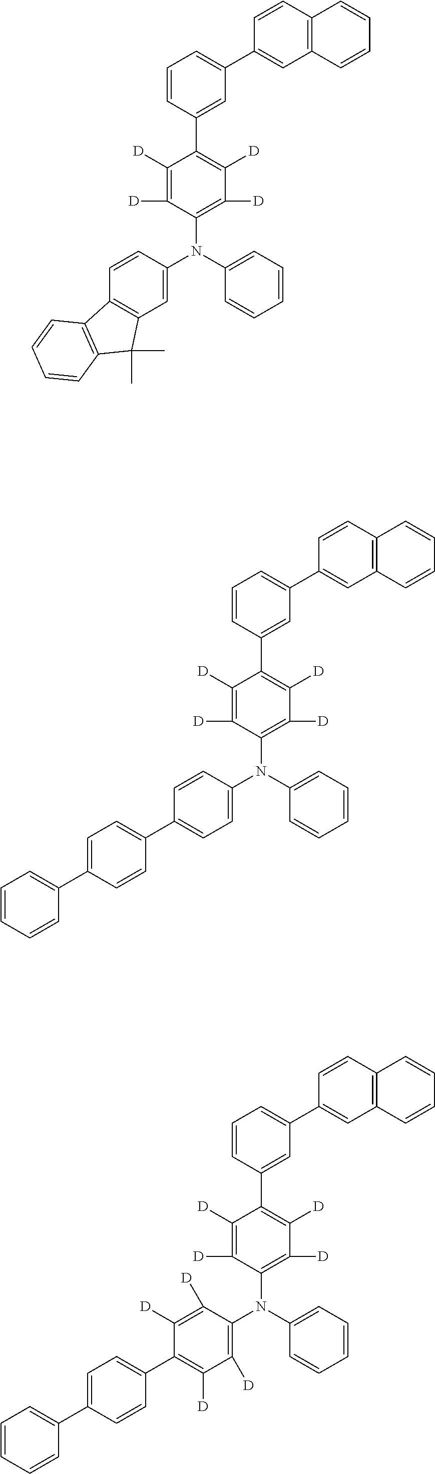 [SMILES: [2H]c1c([2H])c(N(c2ccccc2)c2ccc3c(c2)C(C)(C)c2ccccc2-3)c([2H])c([2H])c1-c1cccc(-c2ccc3ccccc3c2)c1 ; [2H]c1c([2H])c(N(c2ccccc2)c2ccc(-c3ccc(-c4ccccc4)cc3)cc2)c([2H])c([2H])c1-c1cccc(-c2ccc3ccccc3c2)c1 ; [2H]c1c([2H])c(N(c2ccccc2)c2c([2H])c([2H])c(-c3cccc(-c4ccc5ccccc5c4)c3)c([2H])c2[2H])c([2H])c([2H])c1-c1ccc(-c2ccccc2)cc1]

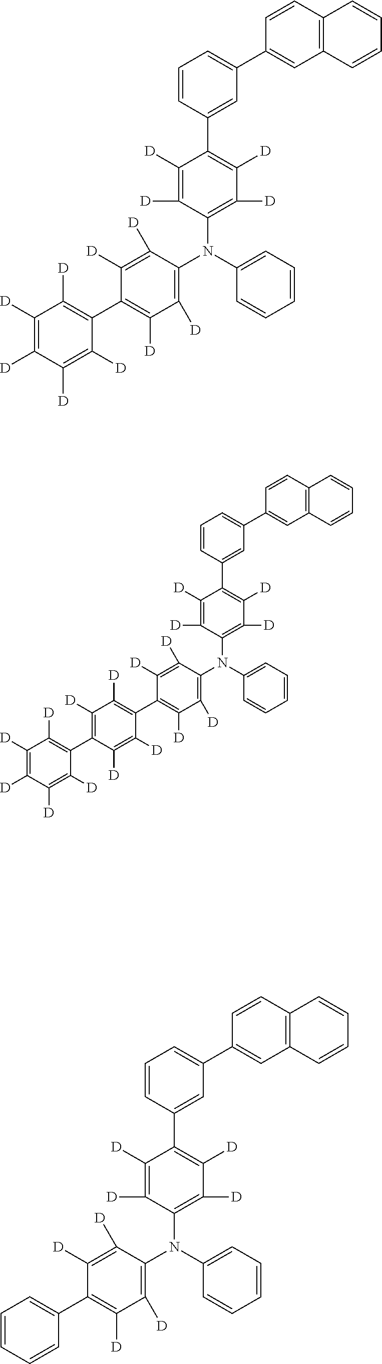 [SMILES: [2H]c1c([2H])c([2H])c(-c2c([2H])c([2H])c(N(c3ccccc3)c3c([2H])c([2H])c(-c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccccc4)c4c([2H])c([2H])c(-c5cccc(-c6ccc7ccccc7c6)c5)c([2H])c4[2H])c([2H])c3[2H])c([2H])c2[2H])c([2H])c1[2H] ; [2H]c1c([2H])c(N(c2ccccc2)c2c([2H])c([2H])c(-c3cccc(-c4ccc5ccccc5c4)c3)c([2H])c2[2H])c([2H])c([2H])c1-c1ccccc1]

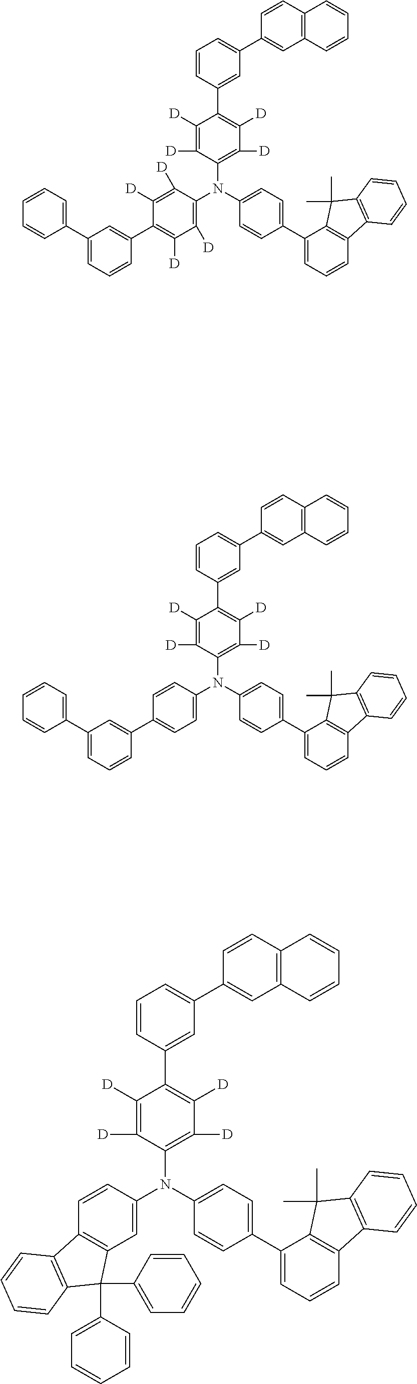 [SMILES: [2H]c1c([2H])c(N(c2ccc(-c3cccc4c3C(C)(C)c3ccccc3-4)cc2)c2c([2H])c([2H])c(-c3cccc(-c4ccc5ccccc5c4)c3)c([2H])c2[2H])c([2H])c([2H])c1-c1cccc(-c2ccccc2)c1 ; [2H]c1c([2H])c(N(c2ccc(-c3cccc(-c4ccccc4)c3)cc2)c2ccc(-c3cccc4c3C(C)(C)c3ccccc3-4)cc2)c([2H])c([2H])c1-c1cccc(-c2ccc3ccccc3c2)c1 ; [2H]c1c([2H])c(N(c2ccc(-c3cccc4c3C(C)(C)c3ccccc3-4)cc2)c2ccc3c(c2)C(c2ccccc2)(c2ccccc2)c2ccccc2-3)c([2H])c([2H])c1-c1cccc(-c2ccc3ccccc3c2)c1]

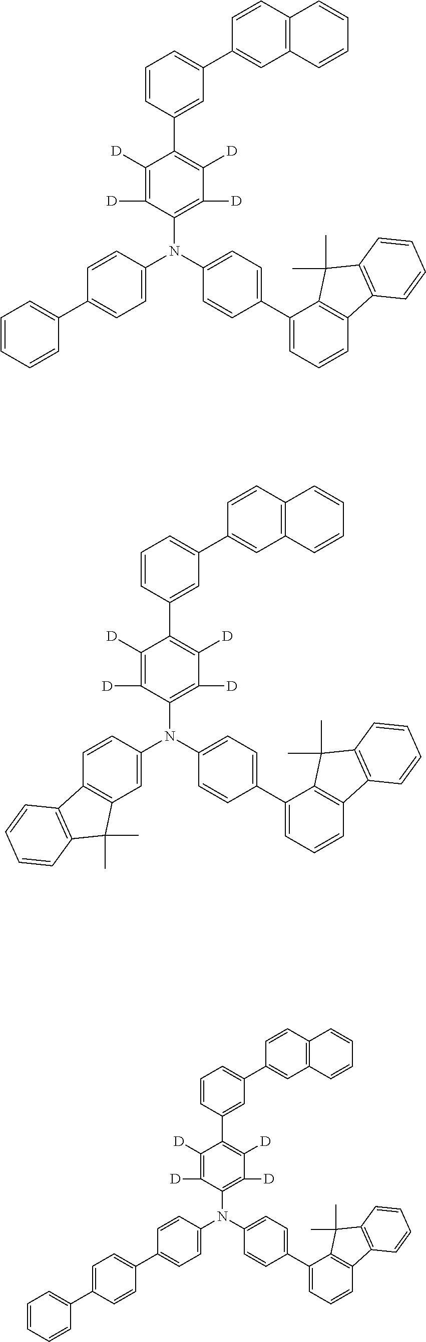 [SMILES: [2H]c1c([2H])c(N(c2ccc(-c3ccccc3)cc2)c2ccc(-c3cccc4c3C(C)(C)c3ccccc3-4)cc2)c([2H])c([2H])c1-c1cccc(-c2ccc3ccccc3c2)c1 ; [2H]c1c([2H])c(N(c2ccc(-c3cccc4c3C(C)(C)c3ccccc3-4)cc2)c2ccc3c(c2)C(C)(C)c2ccccc2-3)c([2H])c([2H])c1-c1cccc(-c2ccc3ccccc3c2)c1 ; [2H]c1c([2H])c(N(c2ccc(-c3ccc(-c4ccccc4)cc3)cc2)c2ccc(-c3cccc4c3C(C)(C)c3ccccc3-4)cc2)c([2H])c([2H])c1-c1cccc(-c2ccc3ccccc3c2)c1]

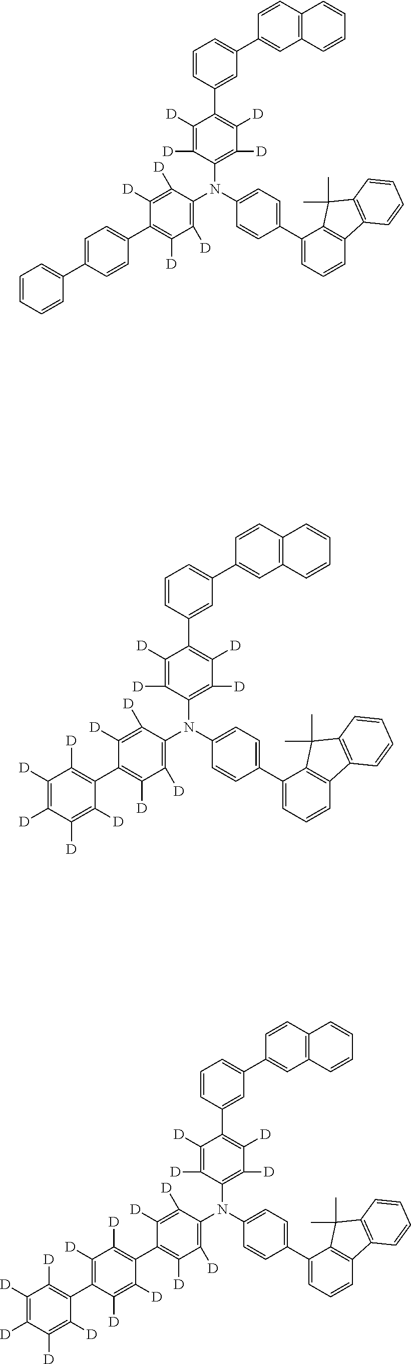

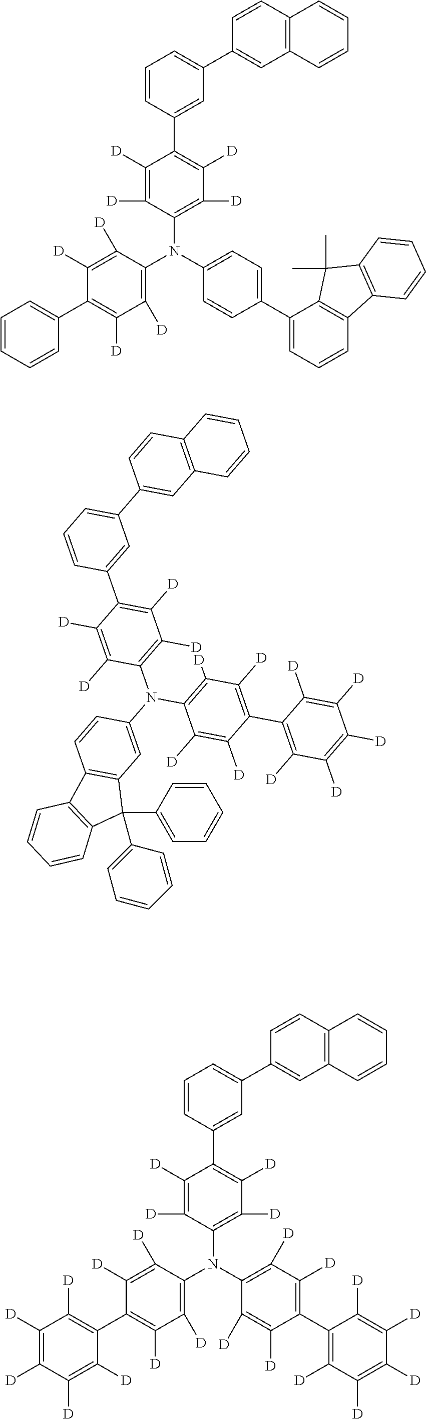

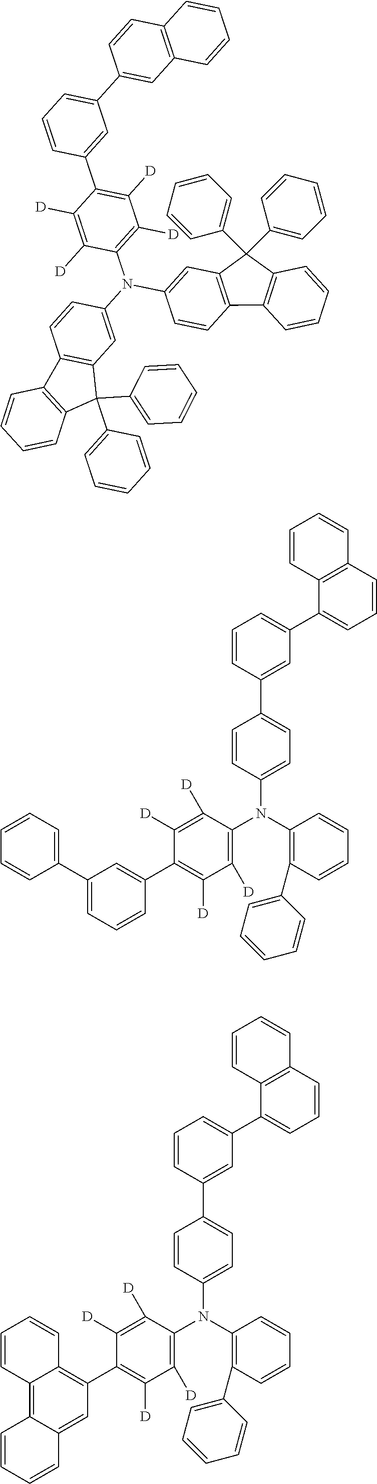 [SMILES: [2H]c1c([2H])c(N(c2ccc3c(c2)C(c2ccccc2)(c2ccccc2)c2ccccc2-3)c2ccc3c(c2)C(c2ccccc2)(c2ccccc2)c2ccccc2-3)c([2H])c([2H])c1-c1cccc(-c2ccc3ccccc3c2)c1 ; [2H]c1c([2H])c(N(c2ccc(-c3cccc(-c4cccc5ccccc45)c3)cc2)c2ccccc2-c2ccccc2)c([2H])c([2H])c1-c1cccc(-c2ccccc2)c1 ; [2H]c1c([2H])c(N(c2ccc(-c3cccc(-c4cccc5ccccc45)c3)cc2)c2ccccc2-c2ccccc2)c([2H])c([2H])c1-c1cc2ccccc2c2ccccc12]

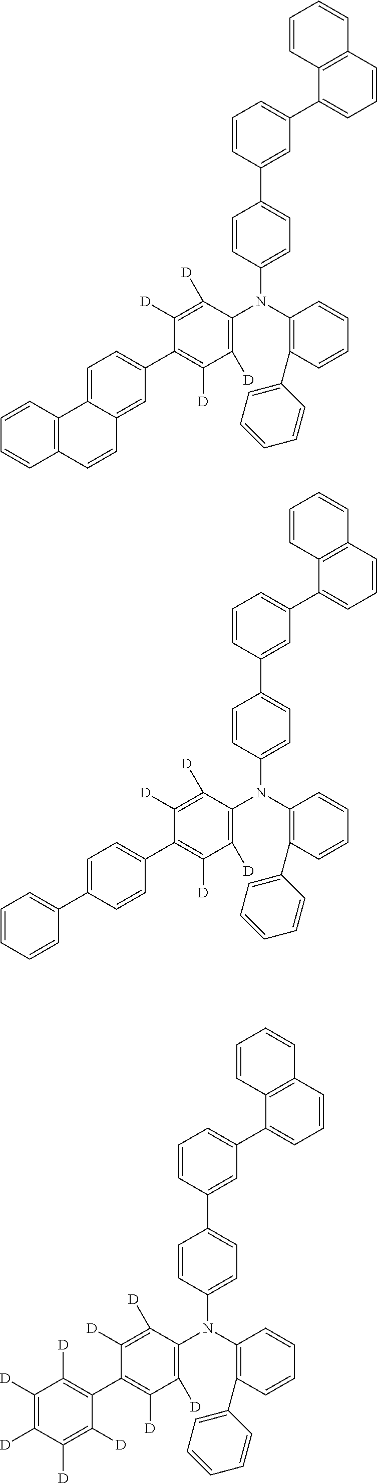 [SMILES: [2H]c1c([2H])c(N(c2ccc(-c3cccc(-c4cccc5ccccc45)c3)cc2)c2ccccc2-c2ccccc2)c([2H])c([2H])c1-c1ccc2c(ccc3ccccc32)c1 ; [2H]c1c([2H])c(N(c2ccc(-c3cccc(-c4cccc5ccccc45)c3)cc2)c2ccccc2-c2ccccc2)c([2H])c([2H])c1-c1ccc(-c2ccccc2)cc1 ; [2H]c1c([2H])c([2H])c(-c2c([2H])c([2H])c(N(c3ccc(-c4cccc(-c5cccc6ccccc56)c4)cc3)c3ccccc3-c3ccccc3)c([2H])c2[2H])c([2H])c1[2H]]

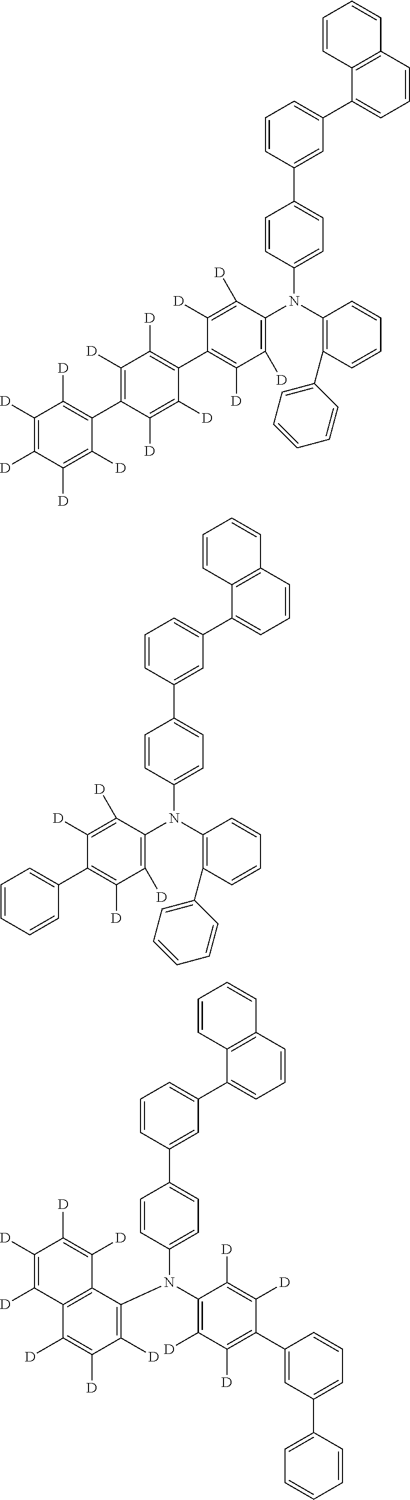

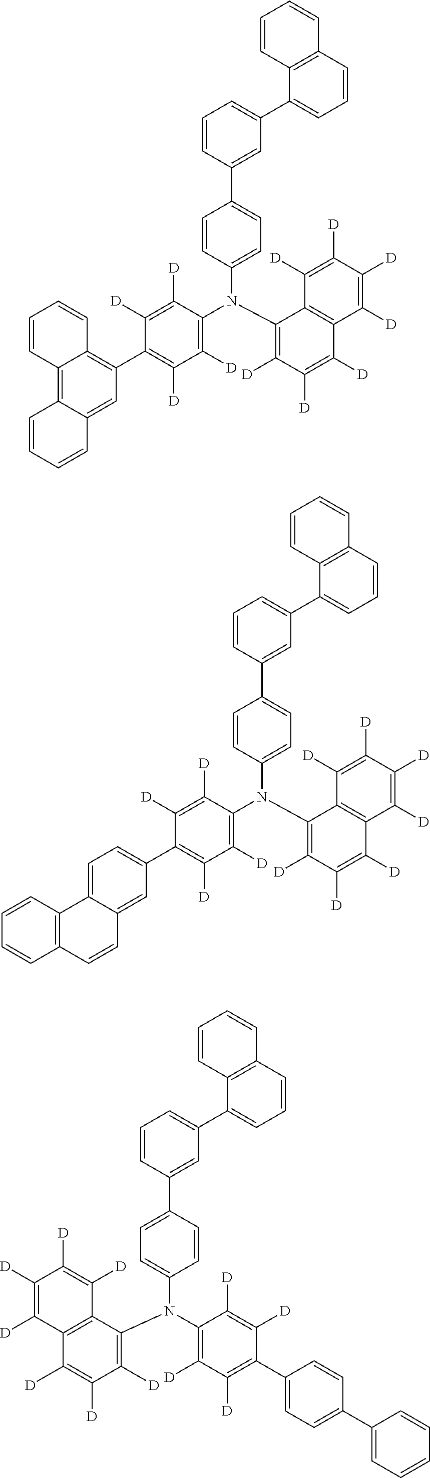 [SMILES: [2H]c1c([2H])c(N(c2ccc(-c3cccc(-c4cccc5ccccc45)c3)cc2)c2c([2H])c([2H])c([2H])c3c([2H])c([2H])c([2H])c([2H])c23)c([2H])c([2H])c1-c1cc2ccccc2c2ccccc12 ; [2H]c1c([2H])c(N(c2ccc(-c3cccc(-c4cccc5ccccc45)c3)cc2)c2c([2H])c([2H])c([2H])c3c([2H])c([2H])c([2H])c([2H])c23)c([2H])c([2H])c1-c1ccc2c(ccc3ccccc32)c1 ; [2H]c1c([2H])c(N(c2ccc(-c3cccc(-c4cccc5ccccc45)c3)cc2)c2c([2H])c([2H])c([2H])c3c([2H])c([2H])c([2H])c([2H])c23)c([2H])c([2H])c1-c1ccc(-c2ccccc2)cc1]

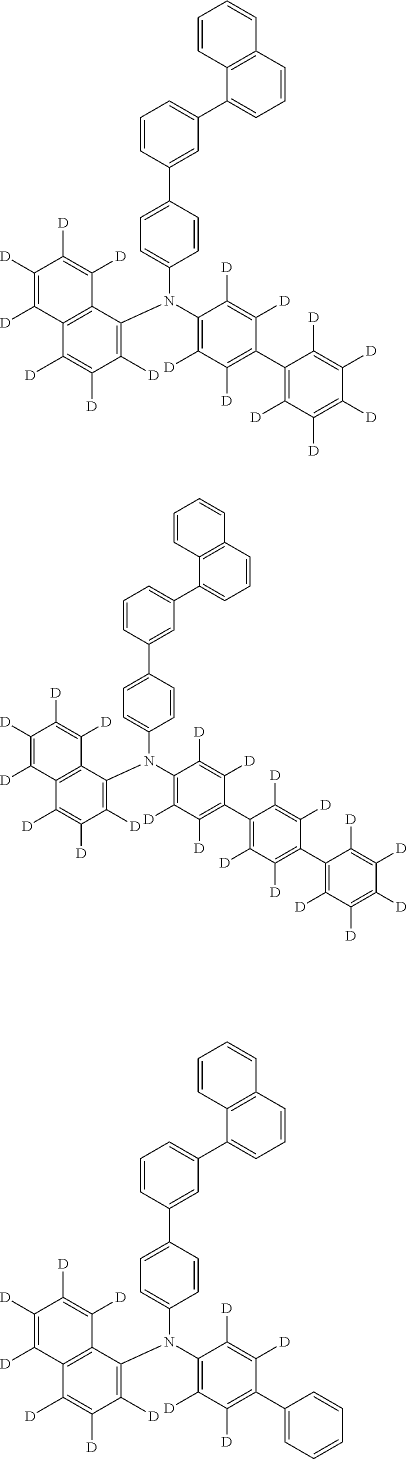

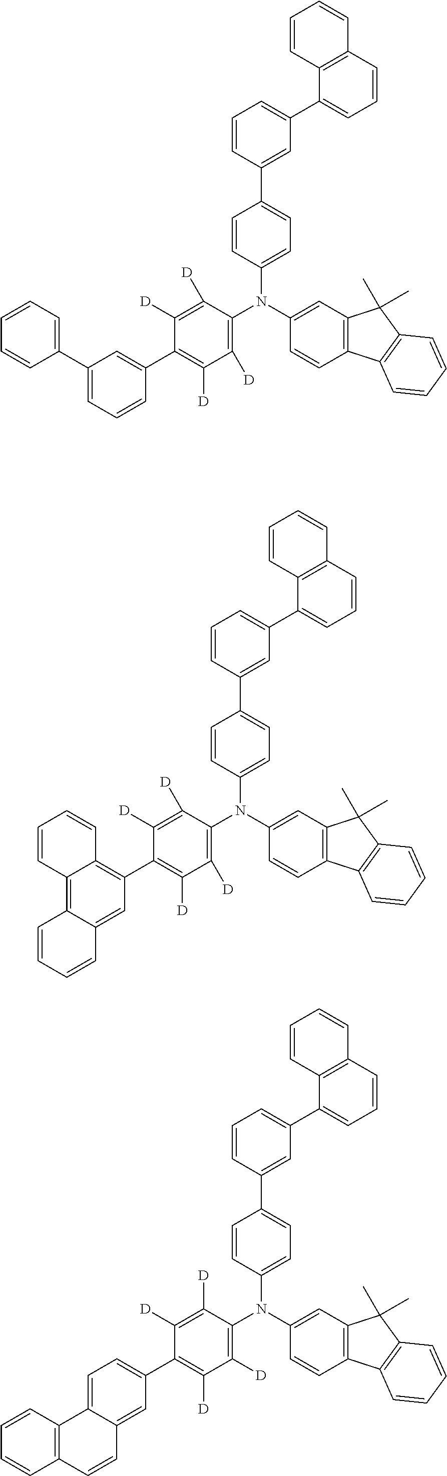 [SMILES: [2H]c1c([2H])c(N(c2ccc(-c3cccc(-c4cccc5ccccc45)c3)cc2)c2ccc3c(c2)C(C)(C)c2ccccc2-3)c([2H])c([2H])c1-c1cccc(-c2ccccc2)c1 ; [2H]c1c([2H])c(N(c2ccc(-c3cccc(-c4cccc5ccccc45)c3)cc2)c2ccc3c(c2)C(C)(C)c2ccccc2-3)c([2H])c([2H])c1-c1cc2ccccc2c2ccccc12 ; [2H]c1c([2H])c(N(c2ccc(-c3cccc(-c4cccc5ccccc45)c3)cc2)c2ccc3c(c2)C(C)(C)c2ccccc2-3)c([2H])c([2H])c1-c1ccc2c(ccc3ccccc32)c1]

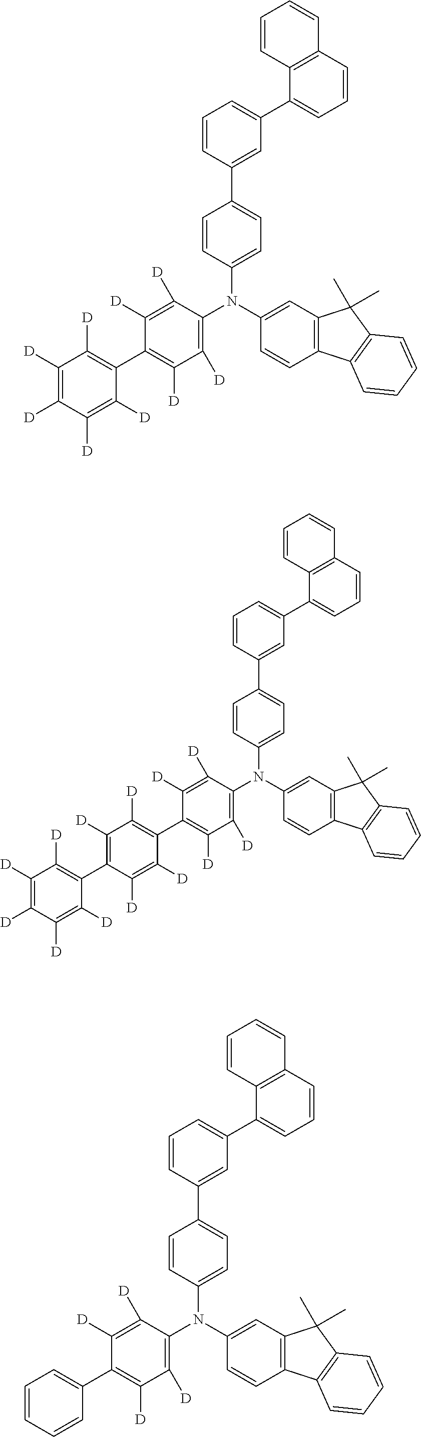 [SMILES: [2H]c1c([2H])c([2H])c(-c2c([2H])c([2H])c(N(c3ccc(-c4cccc(-c5cccc6ccccc56)c4)cc3)c3ccc4c(c3)C(C)(C)c3ccccc3-4)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6cccc7ccccc67)c5)cc4)c4ccc5c(c4)C(C)(C)c4ccccc4-5)c([2H])c3[2H])c([2H])c2[2H])c([2H])c1[2H] ; [2H]c1c([2H])c(N(c2ccc(-c3cccc(-c4cccc5ccccc45)c3)cc2)c2ccc3c(c2)C(C)(C)c2ccccc2-3)c([2H])c([2H])c1-c1ccccc1]

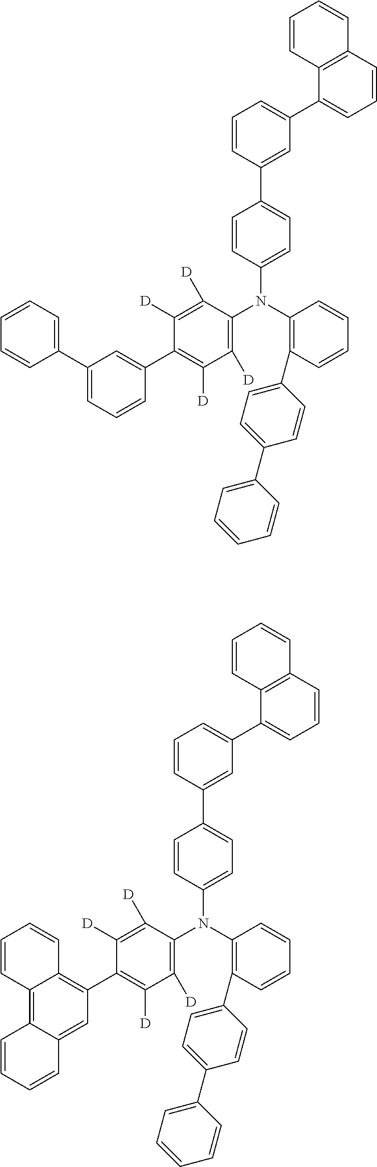 [SMILES: [2H]c1c([2H])c(N(c2ccc(-c3cccc(-c4cccc5ccccc45)c3)cc2)c2ccccc2-c2ccc(-c3ccccc3)cc2)c([2H])c([2H])c1-c1cccc(-c2ccccc2)c1 ; [2H]c1c([2H])c(N(c2ccc(-c3cccc(-c4cccc5ccccc45)c3)cc2)c2ccccc2-c2ccc(-c3ccccc3)cc2)c([2H])c([2H])c1-c1cc2ccccc2c2ccccc12]

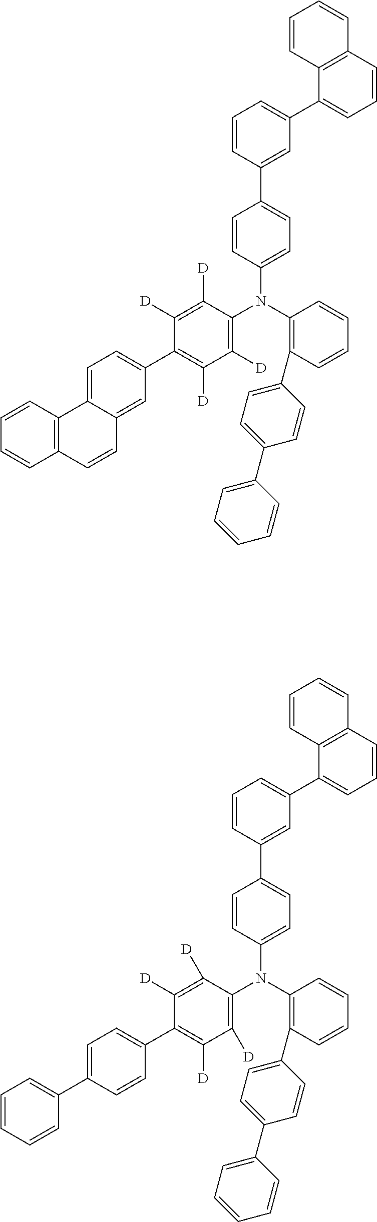 [SMILES: [2H]c1c([2H])c(N(c2ccc(-c3cccc(-c4cccc5ccccc45)c3)cc2)c2ccccc2-c2ccc(-c3ccccc3)cc2)c([2H])c([2H])c1-c1ccc2c(ccc3ccccc32)c1 ; [2H]c1c([2H])c(N(c2ccc(-c3cccc(-c4cccc5ccccc45)c3)cc2)c2ccccc2-c2ccc(-c3ccccc3)cc2)c([2H])c([2H])c1-c1ccc(-c2ccccc2)cc1]

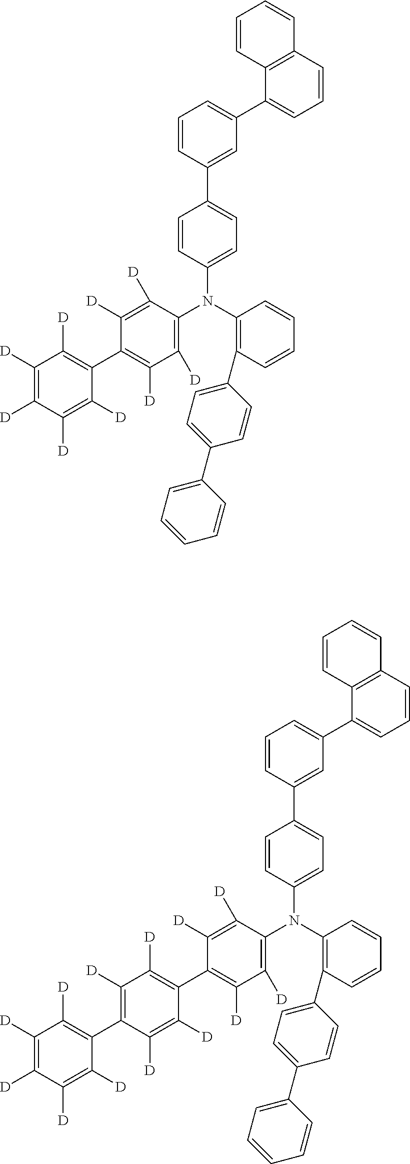 [SMILES: [2H]c1c([2H])c([2H])c(-c2c([2H])c([2H])c(N(c3ccc(-c4cccc(-c5cccc6ccccc56)c4)cc3)c3ccccc3-c3ccc(-c4ccccc4)cc3)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6cccc7ccccc67)c5)cc4)c4ccccc4-c4ccc(-c5ccccc5)cc4)c([2H])c3[2H])c([2H])c2[2H])c([2H])c1[2H]]

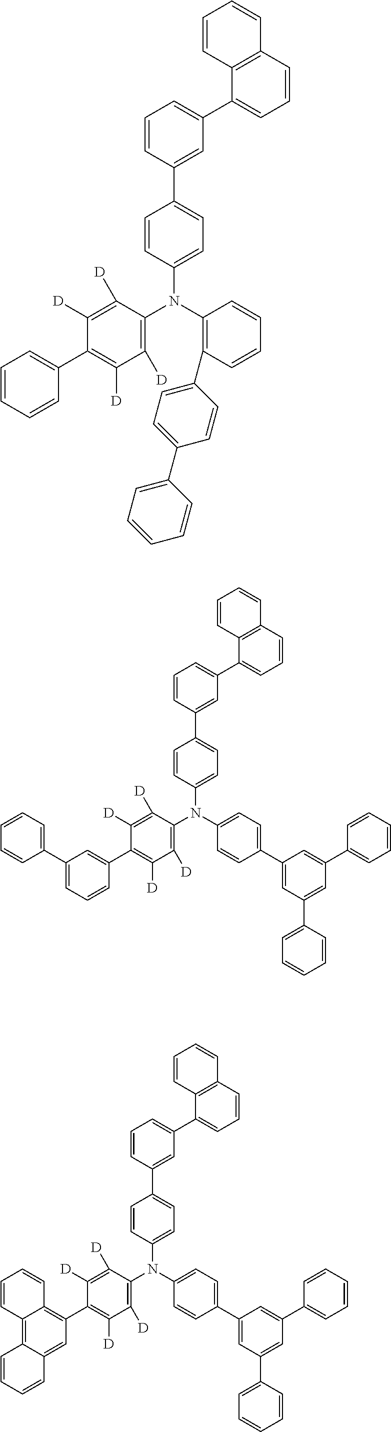 [SMILES: [2H]c1c([2H])c(N(c2ccc(-c3cccc(-c4cccc5ccccc45)c3)cc2)c2ccccc2-c2ccc(-c3ccccc3)cc2)c([2H])c([2H])c1-c1ccccc1 ; [2H]c1c([2H])c(N(c2ccc(-c3cc(-c4ccccc4)cc(-c4ccccc4)c3)cc2)c2ccc(-c3cccc(-c4cccc5ccccc45)c3)cc2)c([2H])c([2H])c1-c1cccc(-c2ccccc2)c1 ; [2H]c1c([2H])c(N(c2ccc(-c3cc(-c4ccccc4)cc(-c4ccccc4)c3)cc2)c2ccc(-c3cccc(-c4cccc5ccccc45)c3)cc2)c([2H])c([2H])c1-c1cc2ccccc2c2ccccc12]

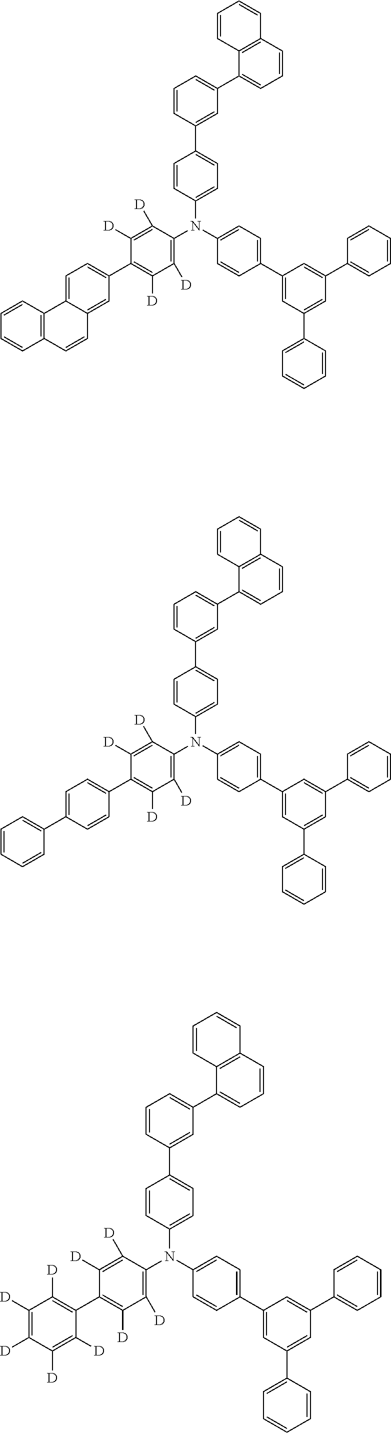 [SMILES: [2H]c1c([2H])c(N(c2ccc(-c3cc(-c4ccccc4)cc(-c4ccccc4)c3)cc2)c2ccc(-c3cccc(-c4cccc5ccccc45)c3)cc2)c([2H])c([2H])c1-c1ccc2c(ccc3ccccc32)c1 ; [2H]c1c([2H])c(N(c2ccc(-c3cc(-c4ccccc4)cc(-c4ccccc4)c3)cc2)c2ccc(-c3cccc(-c4cccc5ccccc45)c3)cc2)c([2H])c([2H])c1-c1ccc(-c2ccccc2)cc1 ; [2H]c1c([2H])c([2H])c(-c2c([2H])c([2H])c(N(c3ccc(-c4cc(-c5ccccc5)cc(-c5ccccc5)c4)cc3)c3ccc(-c4cccc(-c5cccc6ccccc56)c4)cc3)c([2H])c2[2H])c([2H])c1[2H]]

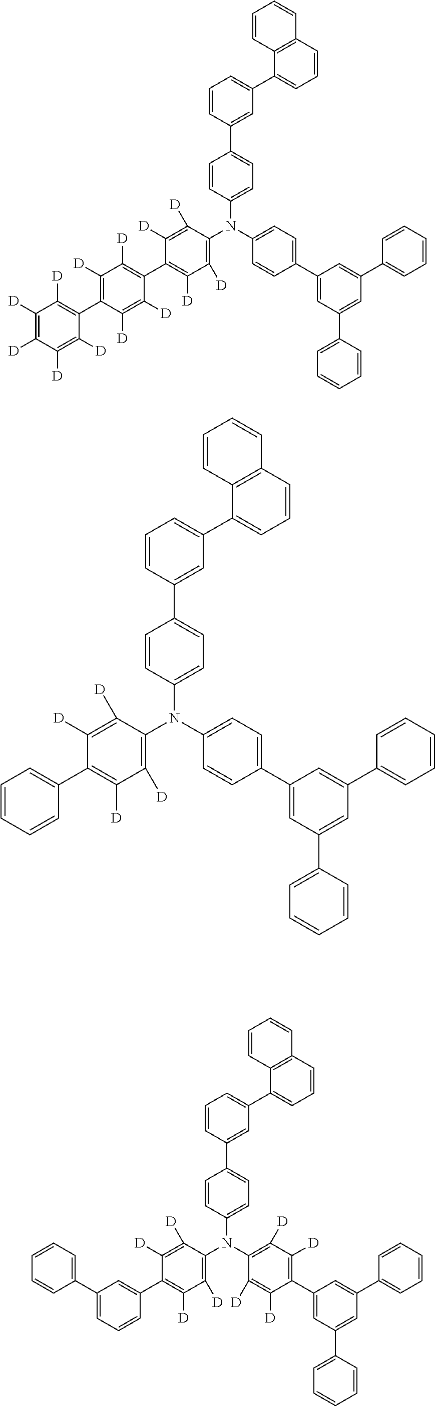 [SMILES: [2H]c1c([2H])c([2H])c(-c2c([2H])c([2H])c(-c3c([2H])c([2H])c(N(c4ccc(-c5cc(-c6ccccc6)cc(-c6ccccc6)c5)cc4)c4ccc(-c5cccc(-c6cccc7ccccc67)c5)cc4)c([2H])c3[2H])c([2H])c2[2H])c([2H])c1[2H] ; [2H]c1c([2H])c(N(c2ccc(-c3cc(-c4ccccc4)cc(-c4ccccc4)c3)cc2)c2ccc(-c3cccc(-c4cccc5ccccc45)c3)cc2)c([2H])c([2H])c1-c1ccccc1 ; [2H]c1c([2H])c(N(c2ccc(-c3cccc(-c4cccc5ccccc45)c3)cc2)c2c([2H])c([2H])c(-c3cc(-c4ccccc4)cc(-c4ccccc4)c3)c([2H])c2[2H])c([2H])c([2H])c1-c1cccc(-c2ccccc2)c1]

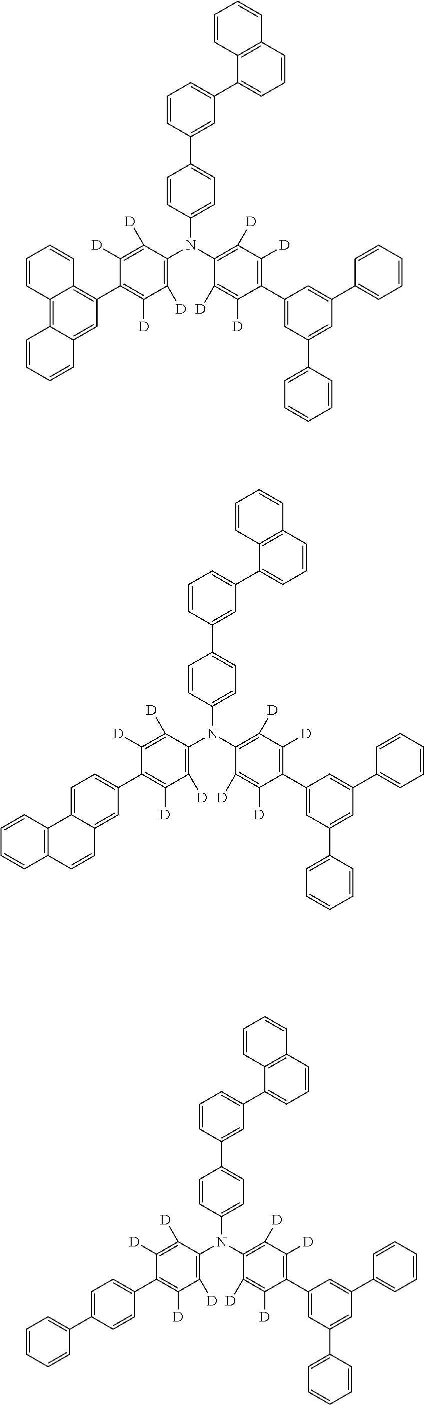 [SMILES: [2H]c1c([2H])c(N(c2ccc(-c3cccc(-c4cccc5ccccc45)c3)cc2)c2c([2H])c([2H])c(-c3cc4ccccc4c4ccccc34)c([2H])c2[2H])c([2H])c([2H])c1-c1cc(-c2ccccc2)cc(-c2ccccc2)c1 ; [2H]c1c([2H])c(N(c2ccc(-c3cccc(-c4cccc5ccccc45)c3)cc2)c2c([2H])c([2H])c(-c3ccc4c(ccc5ccccc54)c3)c([2H])c2[2H])c([2H])c([2H])c1-c1cc(-c2ccccc2)cc(-c2ccccc2)c1 ; [2H]c1c([2H])c(N(c2ccc(-c3cccc(-c4cccc5ccccc45)c3)cc2)c2c([2H])c([2H])c(-c3cc(-c4ccccc4)cc(-c4ccccc4)c3)c([2H])c2[2H])c([2H])c([2H])c1-c1ccc(-c2ccccc2)cc1]

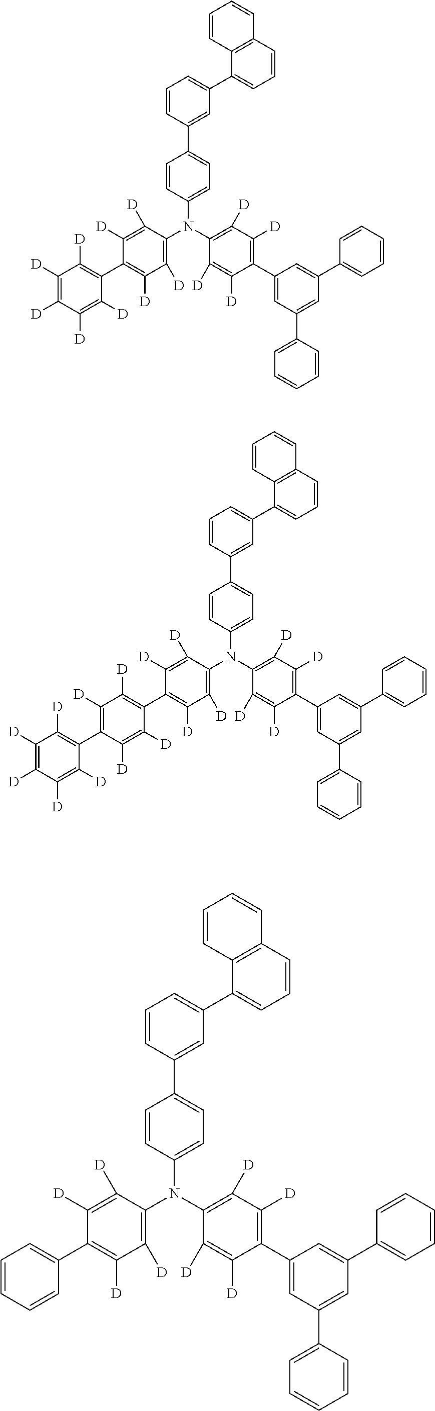

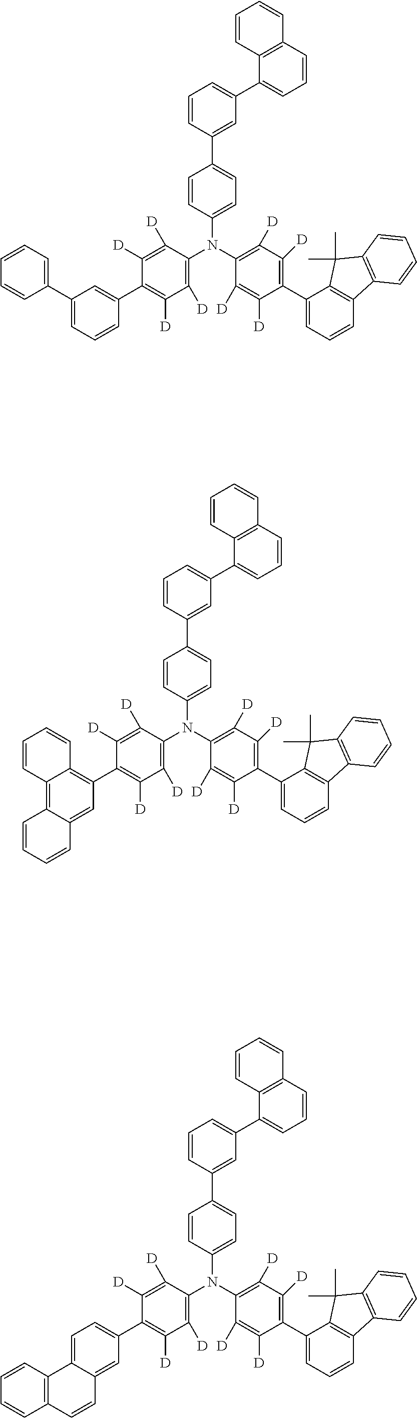 [SMILES: [2H]c1c([2H])c(N(c2ccc(-c3cccc(-c4cccc5ccccc45)c3)cc2)c2c([2H])c([2H])c(-c3cccc4c3C(C)(C)c3ccccc3-4)c([2H])c2[2H])c([2H])c([2H])c1-c1cccc(-c2ccccc2)c1 ; [2H]c1c([2H])c(N(c2ccc(-c3cccc(-c4cccc5ccccc45)c3)cc2)c2c([2H])c([2H])c(-c3cc4ccccc4c4ccccc34)c([2H])c2[2H])c([2H])c([2H])c1-c1cccc2c1C(C)(C)c1ccccc1-2 ; [2H]c1c([2H])c(N(c2ccc(-c3cccc(-c4cccc5ccccc45)c3)cc2)c2c([2H])c([2H])c(-c3cccc4c3C(C)(C)c3ccccc3-4)c([2H])c2[2H])c([2H])c([2H])c1-c1ccc2c(ccc3ccccc32)c1]

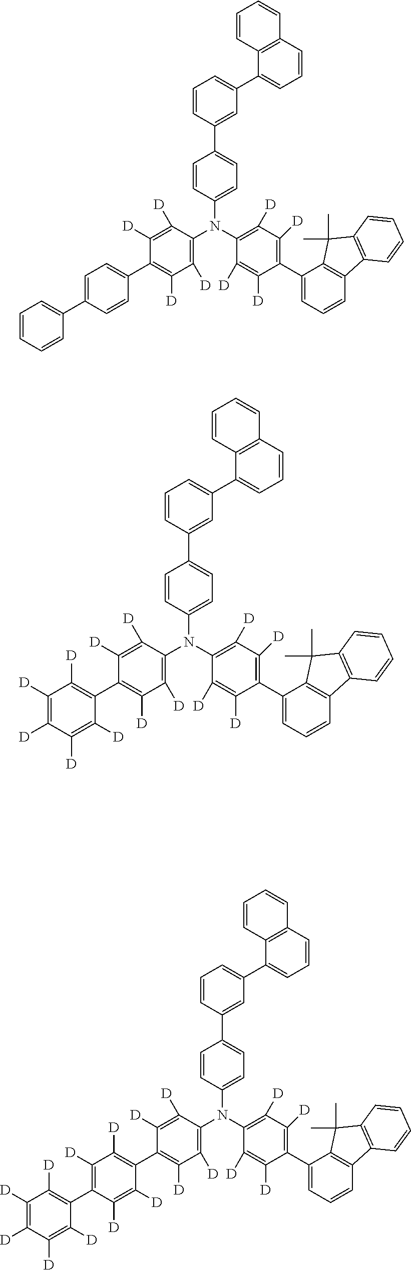

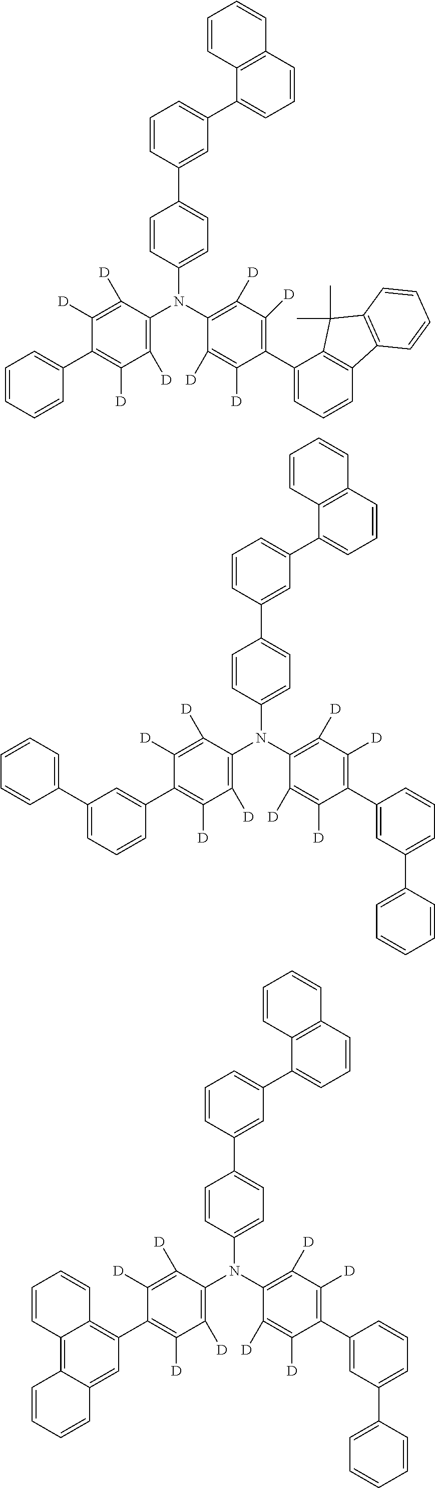

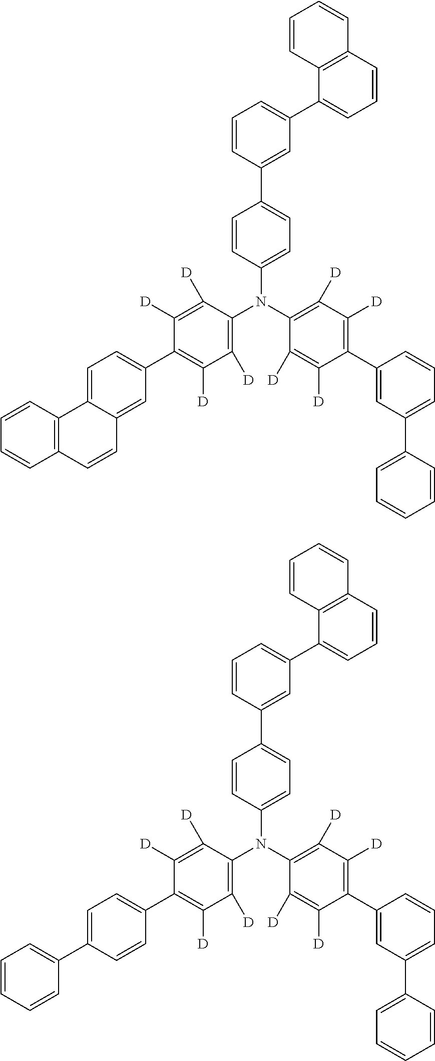 [SMILES: [2H]c1c([2H])c(N(c2ccc(-c3cccc(-c4cccc5ccccc45)c3)cc2)c2c([2H])c([2H])c(-c3ccc4c(ccc5ccccc54)c3)c([2H])c2[2H])c([2H])c([2H])c1-c1cccc(-c2ccccc2)c1 ; [2H]c1c([2H])c(N(c2ccc(-c3cccc(-c4cccc5ccccc45)c3)cc2)c2c([2H])c([2H])c(-c3cccc(-c4ccccc4)c3)c([2H])c2[2H])c([2H])c([2H])c1-c1ccc(-c2ccccc2)cc1]

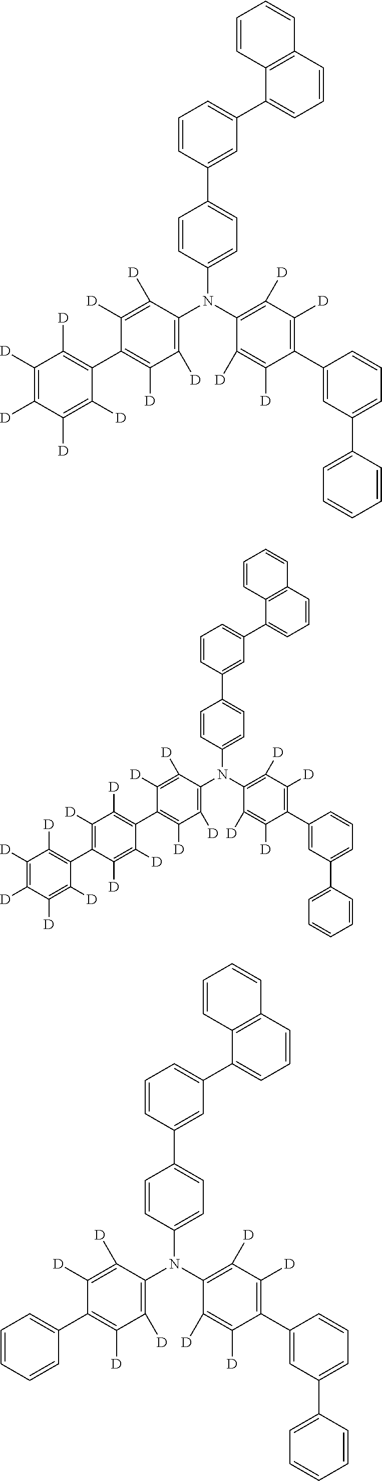 [SMILES: [2H]c1c([2H])c([2H])c(-c2c([2H])c([2H])c(N(c3ccc(-c4cccc(-c5cccc6ccccc56)c4)cc3)c3c([2H])c([2H])c(-c4cccc(-c5ccccc5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6cccc7ccccc67)c5)cc4)c4c([2H])c([2H])c(-c5cccc(-c6ccccc6)c5)c([2H])c4[2H])c([2H])c3[2H])c([2H])c2[2H])c([2H])c1[2H] ; [2H]c1c([2H])c(N(c2ccc(-c3cccc(-c4cccc5ccccc45)c3)cc2)c2c([2H])c([2H])c(-c3cccc(-c4ccccc4)c3)c([2H])c2[2H])c([2H])c([2H])c1-c1ccccc1]

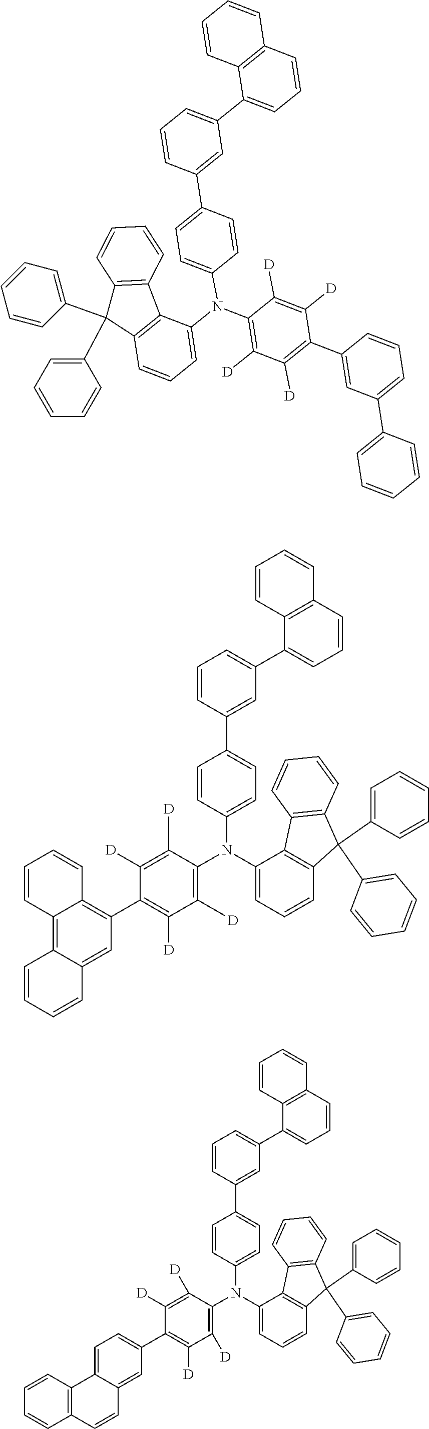 [SMILES: [2H]c1c([2H])c(N(c2ccc(-c3cccc(-c4cccc5ccccc45)c3)cc2)c2cccc3c2-c2ccccc2C3(c2ccccc2)c2ccccc2)c([2H])c([2H])c1-c1cccc(-c2ccccc2)c1 ; [2H]c1c([2H])c(N(c2ccc(-c3cccc(-c4cccc5ccccc45)c3)cc2)c2cccc3c2-c2ccccc2C3(c2ccccc2)c2ccccc2)c([2H])c([2H])c1-c1cc2ccccc2c2ccccc12 ; [2H]c1c([2H])c(N(c2ccc(-c3cccc(-c4cccc5ccccc45)c3)cc2)c2cccc3c2-c2ccccc2C3(c2ccccc2)c2ccccc2)c([2H])c([2H])c1-c1ccc2c(ccc3ccccc32)c1]

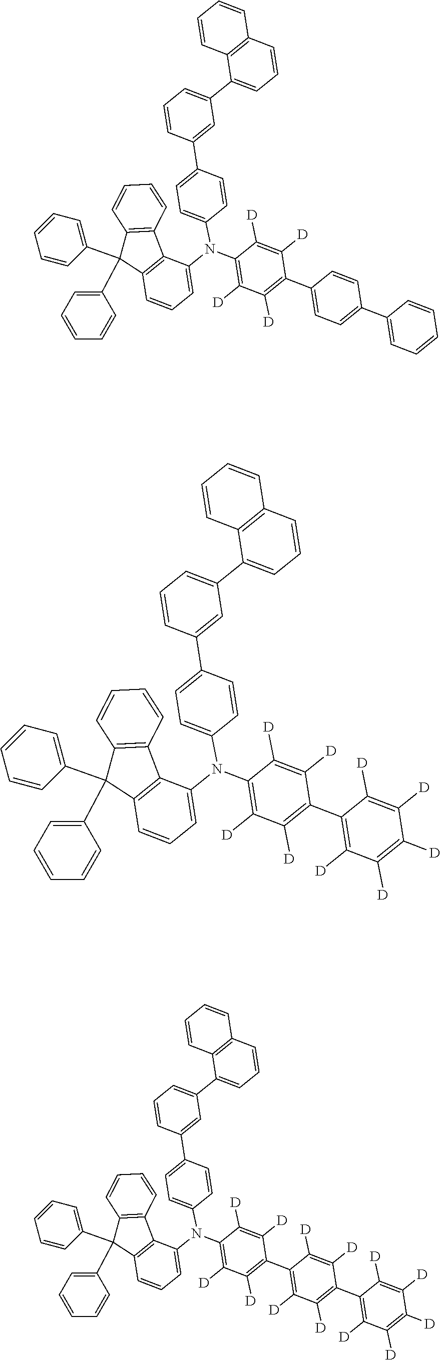 [SMILES: [2H]c1c([2H])c(N(c2ccc(-c3cccc(-c4cccc5ccccc45)c3)cc2)c2cccc3c2-c2ccccc2C3(c2ccccc2)c2ccccc2)c([2H])c([2H])c1-c1ccc(-c2ccccc2)cc1 ; [2H]c1c([2H])c([2H])c(-c2c([2H])c([2H])c(N(c3ccc(-c4cccc(-c5cccc6ccccc56)c4)cc3)c3cccc4c3-c3ccccc3C4(c3ccccc3)c3ccccc3)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6cccc7ccccc67)c5)cc4)c4cccc5c4-c4ccccc4C5(c4ccccc4)c4ccccc4)c([2H])c3[2H])c([2H])c2[2H])c([2H])c1[2H]]

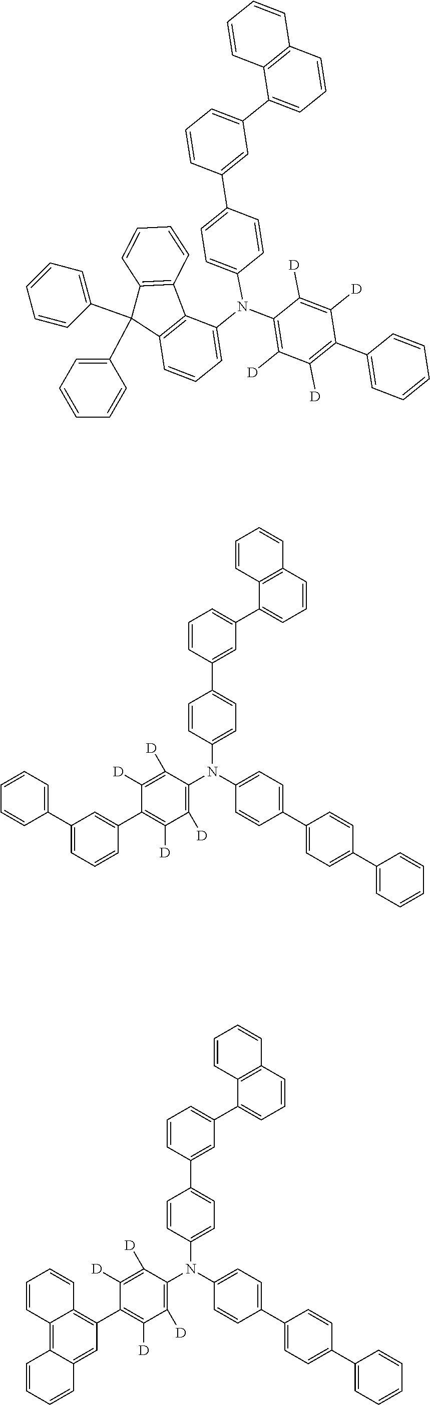 [SMILES: [2H]c1c([2H])c(N(c2ccc(-c3cccc(-c4cccc5ccccc45)c3)cc2)c2cccc3c2-c2ccccc2C3(c2ccccc2)c2ccccc2)c([2H])c([2H])c1-c1ccccc1 ; [2H]c1c([2H])c(N(c2ccc(-c3ccc(-c4ccccc4)cc3)cc2)c2ccc(-c3cccc(-c4cccc5ccccc45)c3)cc2)c([2H])c([2H])c1-c1cccc(-c2ccccc2)c1 ; [2H]c1c([2H])c(N(c2ccc(-c3ccc(-c4ccccc4)cc3)cc2)c2ccc(-c3cccc(-c4cccc5ccccc45)c3)cc2)c([2H])c([2H])c1-c1cc2ccccc2c2ccccc12]

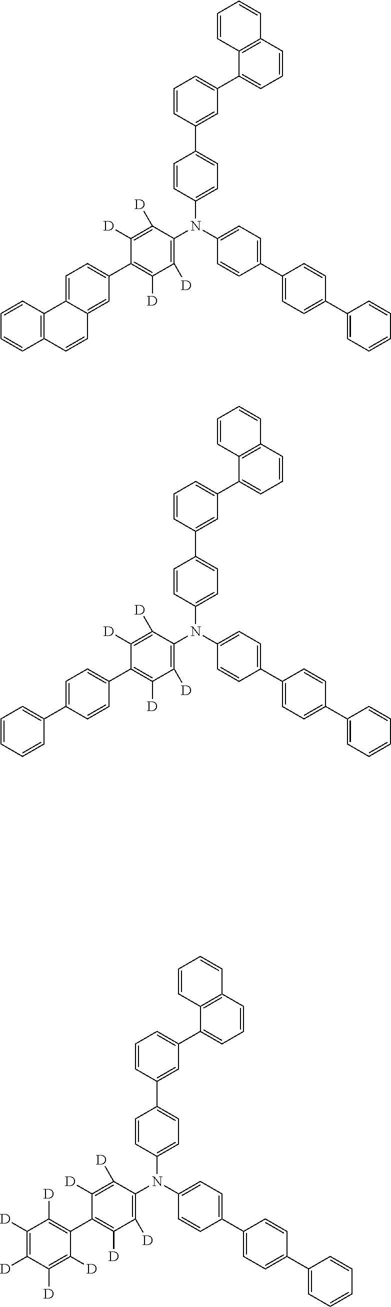 [SMILES: [2H]c1c([2H])c(N(c2ccc(-c3ccc(-c4ccccc4)cc3)cc2)c2ccc(-c3cccc(-c4cccc5ccccc45)c3)cc2)c([2H])c([2H])c1-c1ccc2c(ccc3ccccc32)c1 ; [2H]c1c([2H])c(N(c2ccc(-c3ccc(-c4ccccc4)cc3)cc2)c2ccc(-c3cccc(-c4cccc5ccccc45)c3)cc2)c([2H])c([2H])c1-c1ccc(-c2ccccc2)cc1 ; [2H]c1c([2H])c([2H])c(-c2c([2H])c([2H])c(N(c3ccc(-c4ccc(-c5ccccc5)cc4)cc3)c3ccc(-c4cccc(-c5cccc6ccccc56)c4)cc3)c([2H])c2[2H])c([2H])c1[2H]]

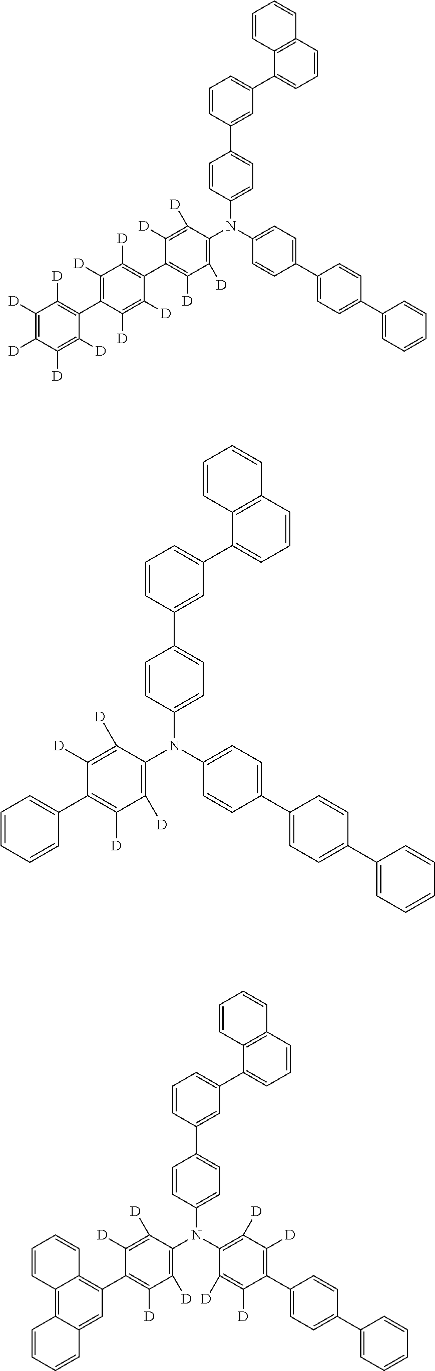 [SMILES: [2H]c1c([2H])c([2H])c(-c2c([2H])c([2H])c(-c3c([2H])c([2H])c(N(c4ccc(-c5ccc(-c6ccccc6)cc5)cc4)c4ccc(-c5cccc(-c6cccc7ccccc67)c5)cc4)c([2H])c3[2H])c([2H])c2[2H])c([2H])c1[2H] ; [2H]c1c([2H])c(N(c2ccc(-c3ccc(-c4ccccc4)cc3)cc2)c2ccc(-c3cccc(-c4cccc5ccccc45)c3)cc2)c([2H])c([2H])c1-c1ccccc1 ; [2H]c1c([2H])c(N(c2ccc(-c3cccc(-c4cccc5ccccc45)c3)cc2)c2c([2H])c([2H])c(-c3cc4ccccc4c4ccccc34)c([2H])c2[2H])c([2H])c([2H])c1-c1ccc(-c2ccccc2)cc1]

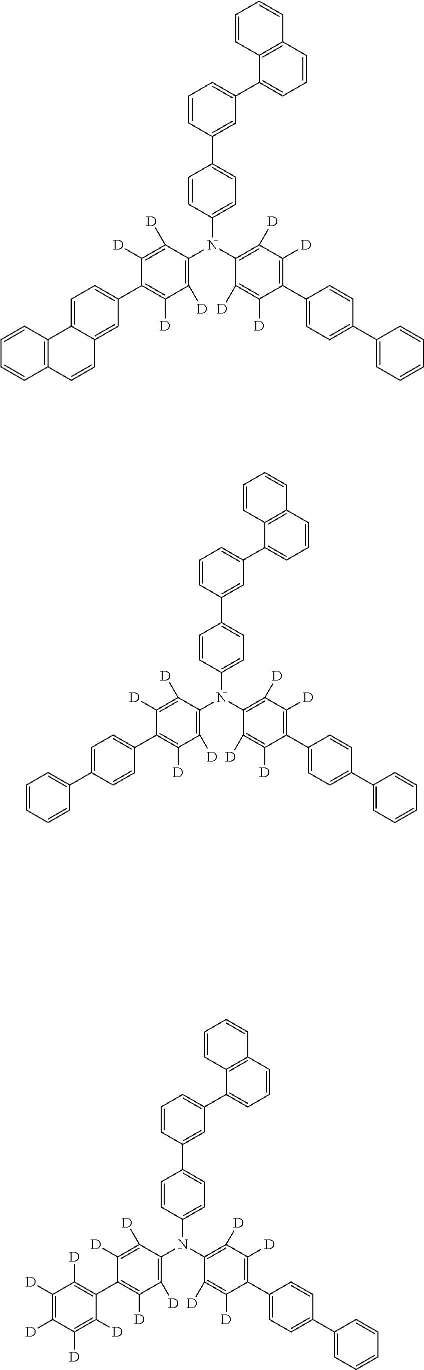 [SMILES: [2H]c1c([2H])c(N(c2ccc(-c3cccc(-c4cccc5ccccc45)c3)cc2)c2c([2H])c([2H])c(-c3ccc4c(ccc5ccccc54)c3)c([2H])c2[2H])c([2H])c([2H])c1-c1ccc(-c2ccccc2)cc1 ; [2H]c1c([2H])c(N(c2ccc(-c3cccc(-c4cccc5ccccc45)c3)cc2)c2c([2H])c([2H])c(-c3ccc(-c4ccccc4)cc3)c([2H])c2[2H])c([2H])c([2H])c1-c1ccc(-c2ccccc2)cc1 ; [2H]c1c([2H])c([2H])c(-c2c([2H])c([2H])c(N(c3ccc(-c4cccc(-c5cccc6ccccc56)c4)cc3)c3c([2H])c([2H])c(-c4ccc(-c5ccccc5)cc4)c([2H])c3[2H])c([2H])c2[2H])c([2H])c1[2H]]

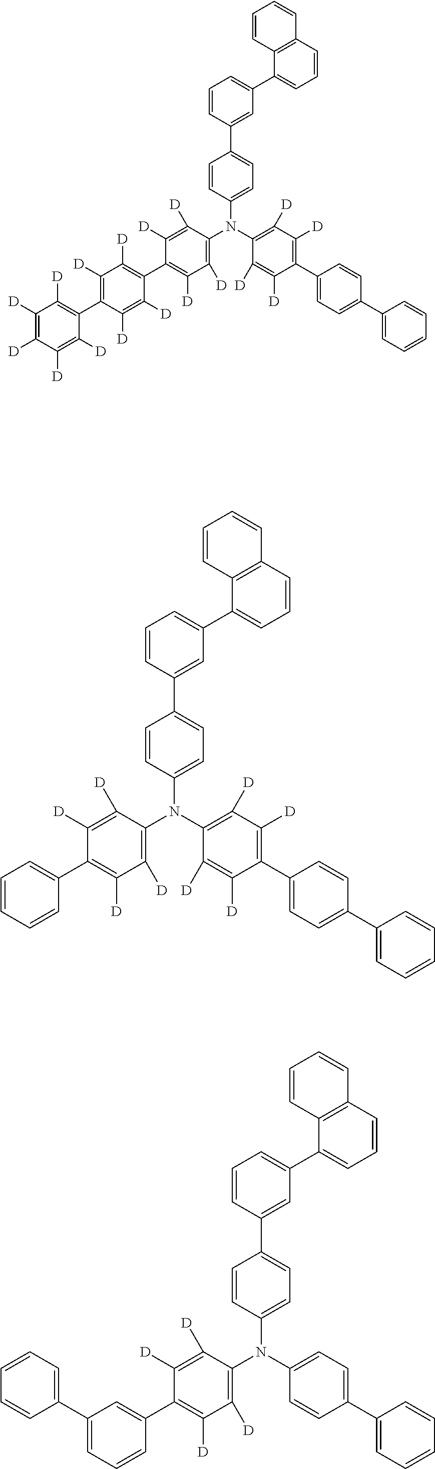 [SMILES: [2H]c1c([2H])c([2H])c(-c2c([2H])c([2H])c(-c3c([2H])c([2H])c(N(c4ccc(-c5cccc(-c6cccc7ccccc67)c5)cc4)c4c([2H])c([2H])c(-c5ccc(-c6ccccc6)cc5)c([2H])c4[2H])c([2H])c3[2H])c([2H])c2[2H])c([2H])c1[2H] ; [2H]c1c([2H])c(N(c2ccc(-c3cccc(-c4cccc5ccccc45)c3)cc2)c2c([2H])c([2H])c(-c3ccc(-c4ccccc4)cc3)c([2H])c2[2H])c([2H])c([2H])c1-c1ccccc1 ; [2H]c1c([2H])c(N(c2ccc(-c3ccccc3)cc2)c2ccc(-c3cccc(-c4cccc5ccccc45)c3)cc2)c([2H])c([2H])c1-c1cccc(-c2ccccc2)c1]

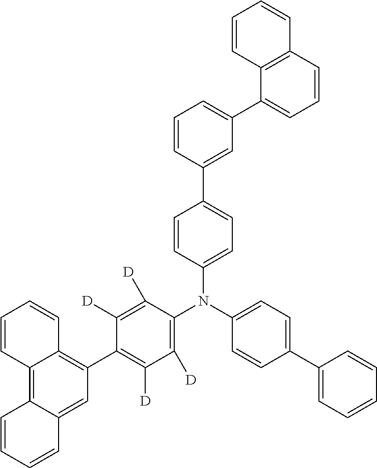 [SMILES: [2H]c1c([2H])c(N(c2ccc(-c3ccccc3)cc2)c2ccc(-c3cccc(-c4cccc5ccccc45)c3)cc2)c([2H])c([2H])c1-c1cc2ccccc2c2ccccc12]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

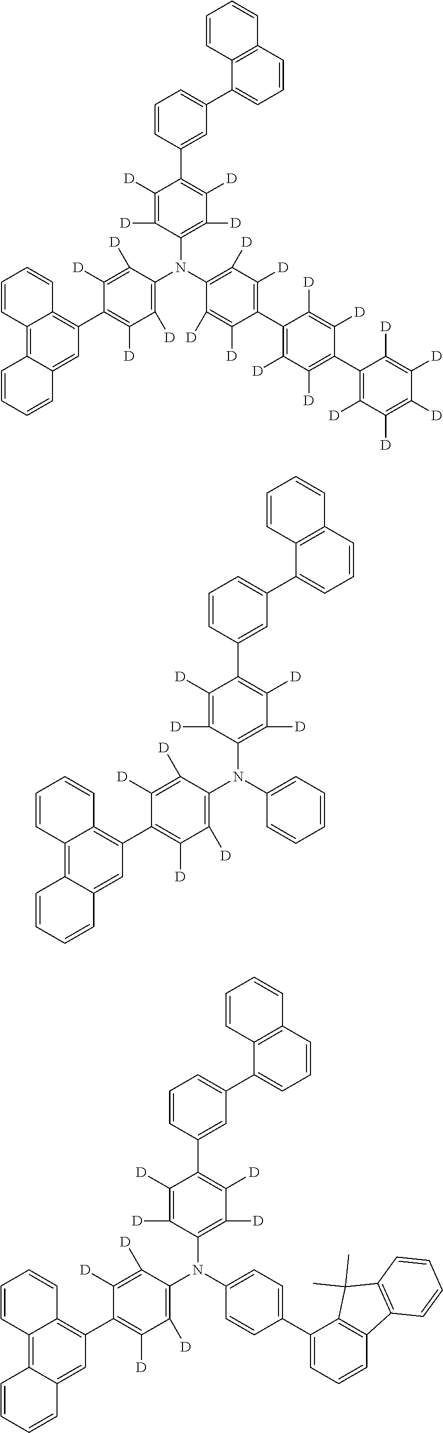 [SMILES: [2H]c1c([2H])c([2H])c(-c2c([2H])c([2H])c(-c3c([2H])c([2H])c(N(c4c([2H])c([2H])c(-c5cccc(-c6cccc7ccccc67)c5)c([2H])c4[2H])c4c([2H])c([2H])c(-c5cc6ccccc6c6ccccc56)c([2H])c4[2H])c([2H])c3[2H])c([2H])c2[2H])c([2H])c1[2H] ; [2H]c1c([2H])c(N(c2ccccc2)c2c([2H])c([2H])c(-c3cc4ccccc4c4ccccc34)c([2H])c2[2H])c([2H])c([2H])c1-c1cccc(-c2cccc3ccccc23)c1 ; [2H]c1c([2H])c(N(c2ccc(-c3cccc4c3C(C)(C)c3ccccc3-4)cc2)c2c([2H])c([2H])c(-c3cc4ccccc4c4ccccc34)c([2H])c2[2H])c([2H])c([2H])c1-c1cccc(-c2cccc3ccccc23)c1]

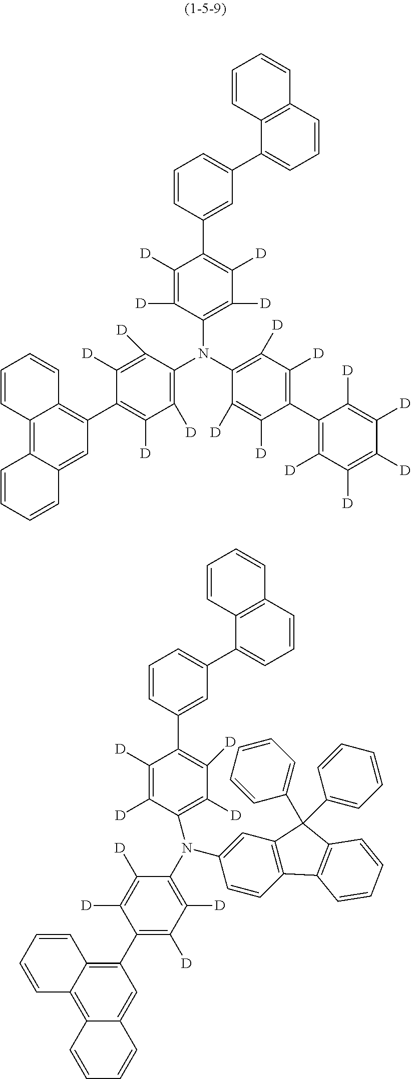 [SMILES: [2H]c1c([2H])c([2H])c(-c2c([2H])c([2H])c(N(c3c([2H])c([2H])c(-c4cccc(-c5cccc6ccccc56)c4)c([2H])c3[2H])c3c([2H])c([2H])c(-c4cc5ccccc5c5ccccc45)c([2H])c3[2H])c([2H])c2[2H])c([2H])c1[2H] ; [2H]c1c([2H])c(N(c2ccc3c(c2)C(c2ccccc2)(c2ccccc2)c2ccccc2-3)c2c([2H])c([2H])c(-c3cc4ccccc4c4ccccc34)c([2H])c2[2H])c([2H])c([2H])c1-c1cccc(-c2cccc3ccccc23)c1]

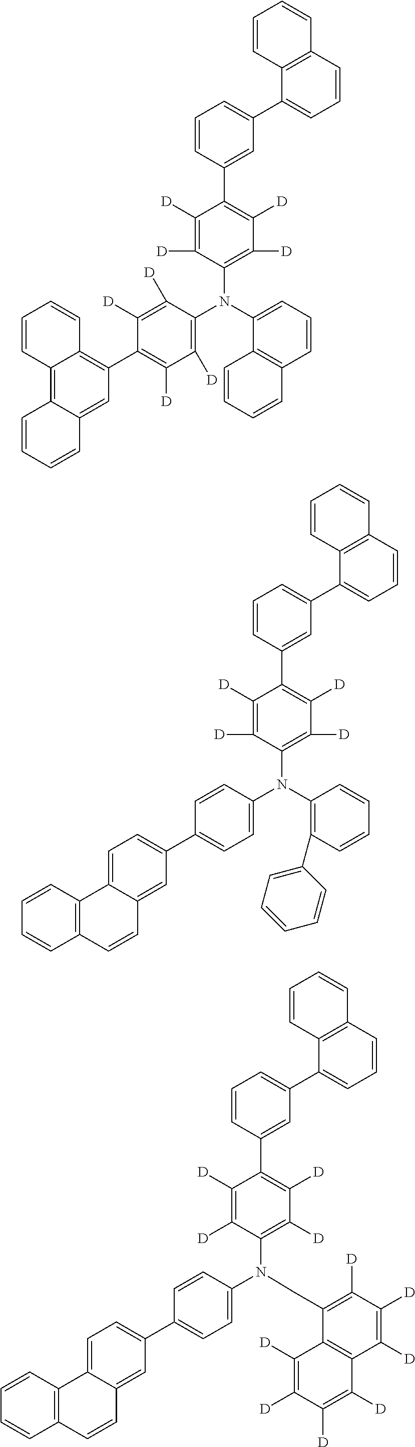 [SMILES: [2H]c1c([2H])c(N(c2c([2H])c([2H])c(-c3cc4ccccc4c4ccccc34)c([2H])c2[2H])c2cccc3ccccc23)c([2H])c([2H])c1-c1cccc(-c2cccc3ccccc23)c1 ; [2H]c1c([2H])c(N(c2ccc(-c3ccc4c(ccc5ccccc54)c3)cc2)c2ccccc2-c2ccccc2)c([2H])c([2H])c1-c1cccc(-c2cccc3ccccc23)c1 ; [2H]c1c([2H])c(N(Cc2c([2H])c([2H])c([2H])c3c([2H])c([2H])c([2H])c([2H])c23)c2ccc(-c3ccc4c(ccc5ccccc54)c3)cc2)c([2H])c([2H])c1-c1cccc(-c2cccc3ccccc23)c1]

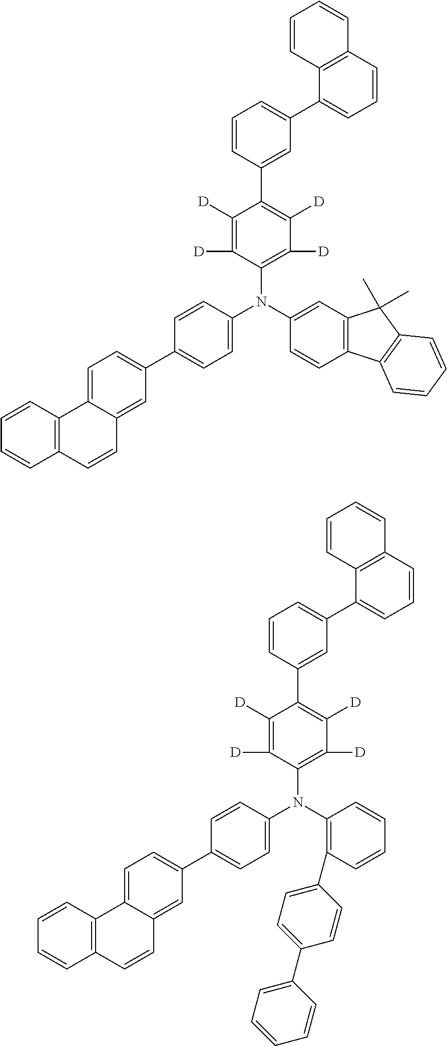 [SMILES: [2H]c1c([2H])c(N(c2ccc(-c3ccc4c(ccc5ccccc54)c3)cc2)c2ccc3c(c2)C(C)(C)c2ccccc2-3)c([2H])c([2H])c1-c1cccc(-c2cccc3ccccc23)c1 ; [2H]c1c([2H])c(N(c2ccc(-c3ccc4c(ccc5ccccc54)c3)cc2)c2ccccc2-c2ccc(-c3ccccc3)cc2)c([2H])c([2H])c1-c1cccc(-c2cccc3ccccc23)c1]

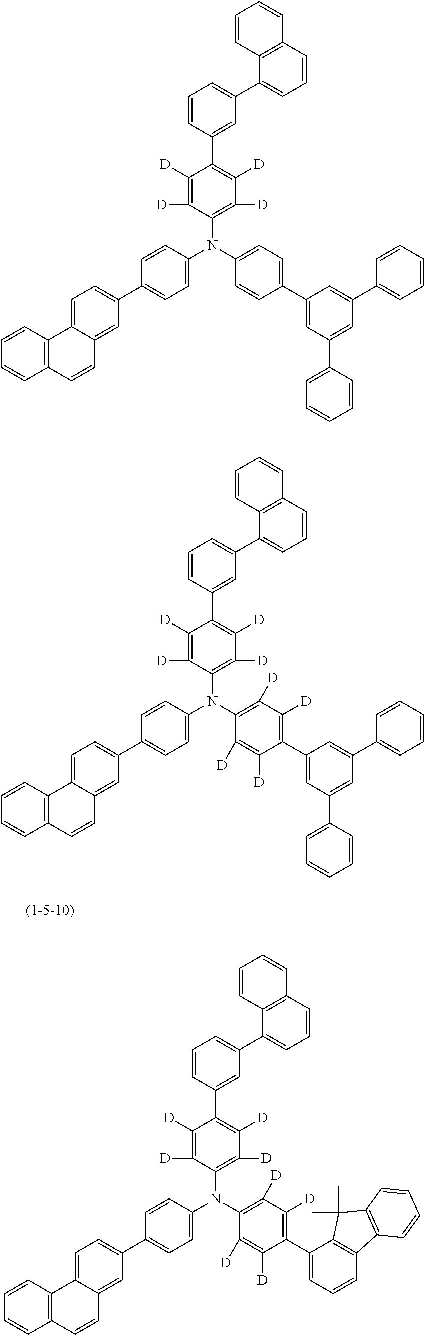 [SMILES: [2H]c1c([2H])c(N(c2ccc(-c3cc(-c4ccccc4)cc(-c4ccccc4)c3)cc2)c2ccc(-c3ccc4c(ccc5ccccc54)c3)cc2)c([2H])c([2H])c1-c1cccc(-c2cccc3ccccc23)c1 ; [2H]c1c([2H])c(N(c2ccc(-c3ccc4c(ccc5ccccc54)c3)cc2)c2c([2H])c([2H])c(-c3cccc(-c4cccc5ccccc45)c3)c([2H])c2[2H])c([2H])c([2H])c1-c1cc(-c2ccccc2)cc(-c2ccccc2)c1 ; [2H]c1c([2H])c(N(c2ccc(-c3ccc4c(ccc5ccccc54)c3)cc2)c2c([2H])c([2H])c(-c3cccc4c3C(C)(C)c3ccccc3-4)c([2H])c2[2H])c([2H])c([2H])c1-c1cccc(-c2cccc3ccccc23)c1]

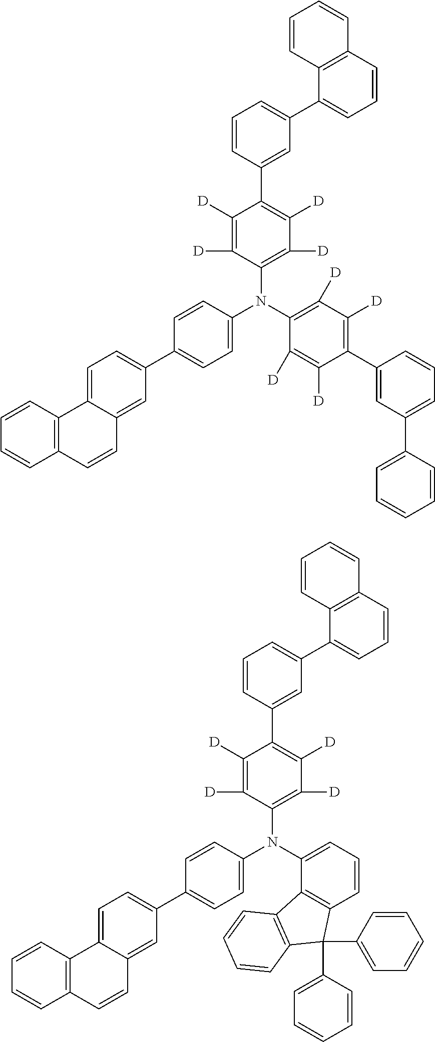 [SMILES: [2H]c1c([2H])c(N(c2ccc(-c3ccc4c(ccc5ccccc54)c3)cc2)c2c([2H])c([2H])c(-c3cccc(-c4cccc5ccccc45)c3)c([2H])c2[2H])c([2H])c([2H])c1-c1cccc(-c2ccccc2)c1 ; [2H]c1c([2H])c(N(c2ccc(-c3ccc4c(ccc5ccccc54)c3)cc2)c2cccc3c2-c2ccccc2C3(c2ccccc2)c2ccccc2)c([2H])c([2H])c1-c1cccc(-c2cccc3ccccc23)c1]

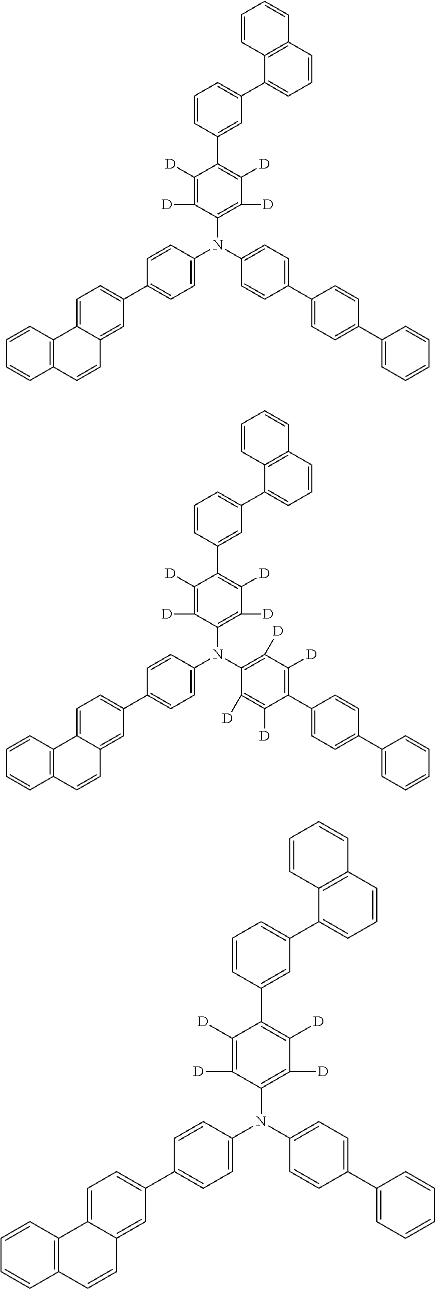 [SMILES: [2H]c1c([2H])c(N(c2ccc(-c3ccc(-c4ccccc4)cc3)cc2)c2ccc(-c3ccc4c(ccc5ccccc54)c3)cc2)c([2H])c([2H])c1-c1cccc(-c2cccc3ccccc23)c1 ; [2H]c1c([2H])c(N(c2ccc(-c3ccc4c(ccc5ccccc54)c3)cc2)c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2ccc(-c3ccccc3)cc2)c2ccc(-c3ccc4c(ccc5ccccc54)c3)cc2)c([2H])c([2H])c1-c1cccc(-c2cccc3ccccc23)c1]

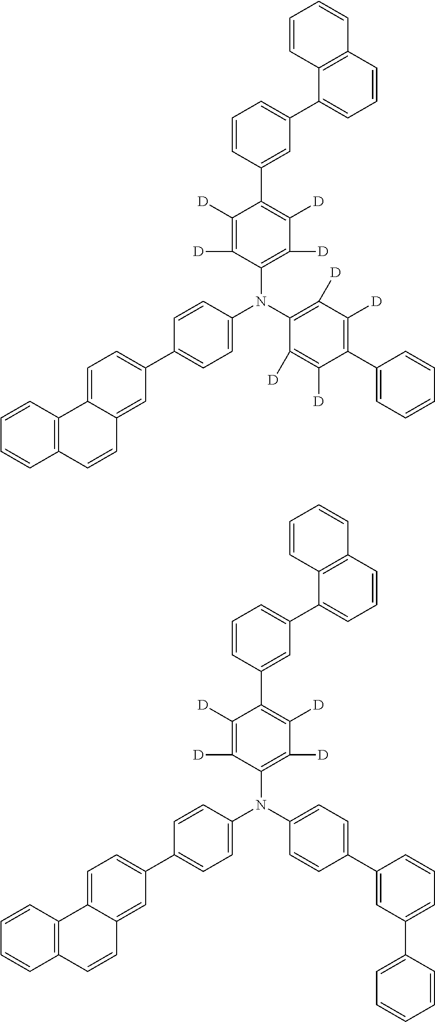 [SMILES: [2H]c1c([2H])c(N(c2ccc(-c3ccc4c(ccc5ccccc54)c3)cc2)c2c([2H])c([2H])c(-c3cccc(-c4cccc5ccccc45)c3)c([2H])c2[2H])c([2H])c([2H])c1-c1ccccc1 ; [2H]c1c([2H])c(N(c2ccc(-c3cccc(-c4ccccc4)c3)cc2)c2ccc(-c3ccc4c(ccc5ccccc54)c3)cc2)c([2H])c([2H])c1-c1cccc(-c2cccc3ccccc23)c1]

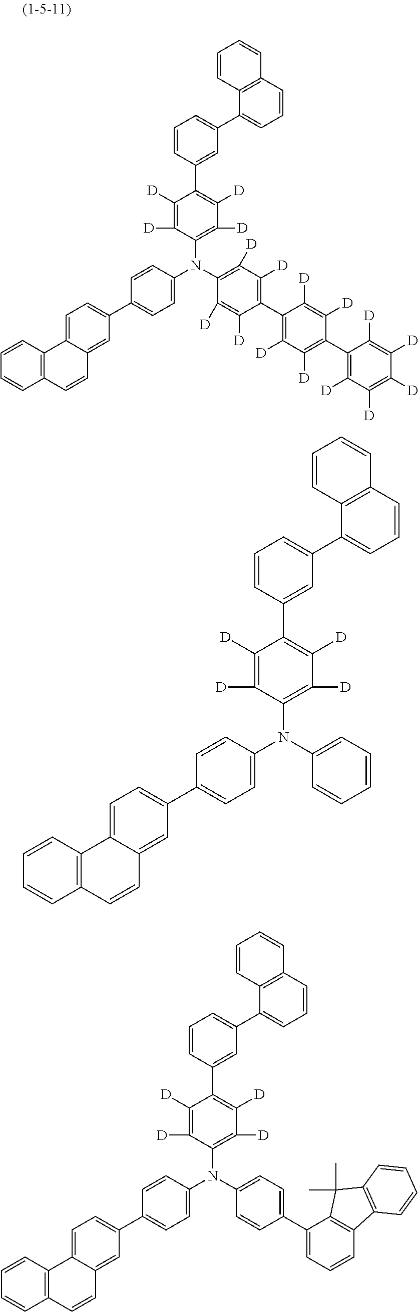

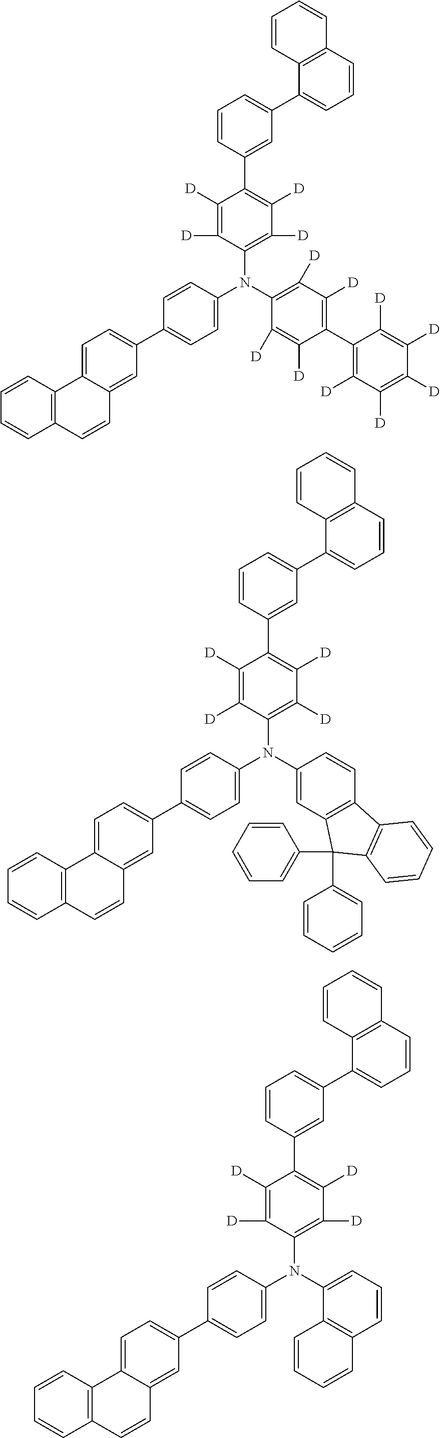

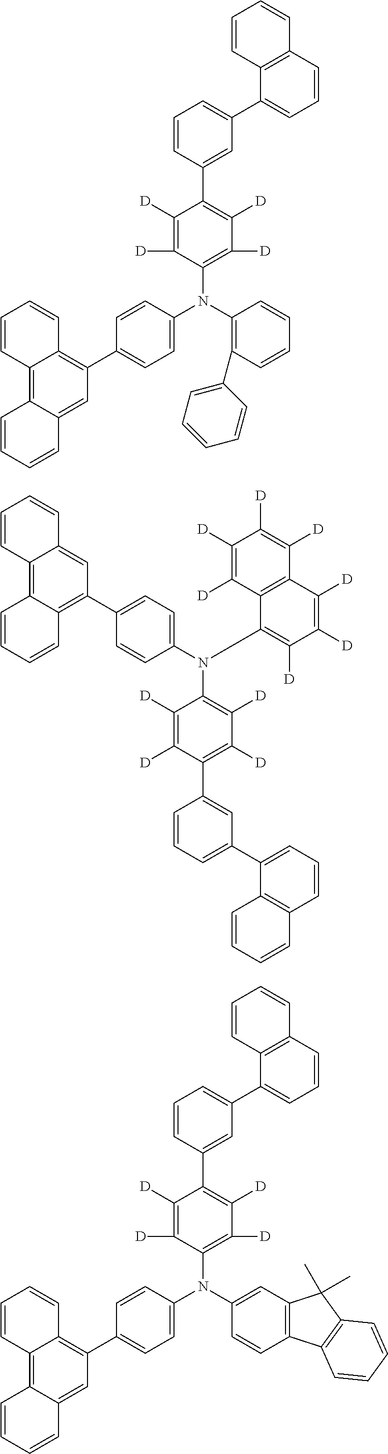

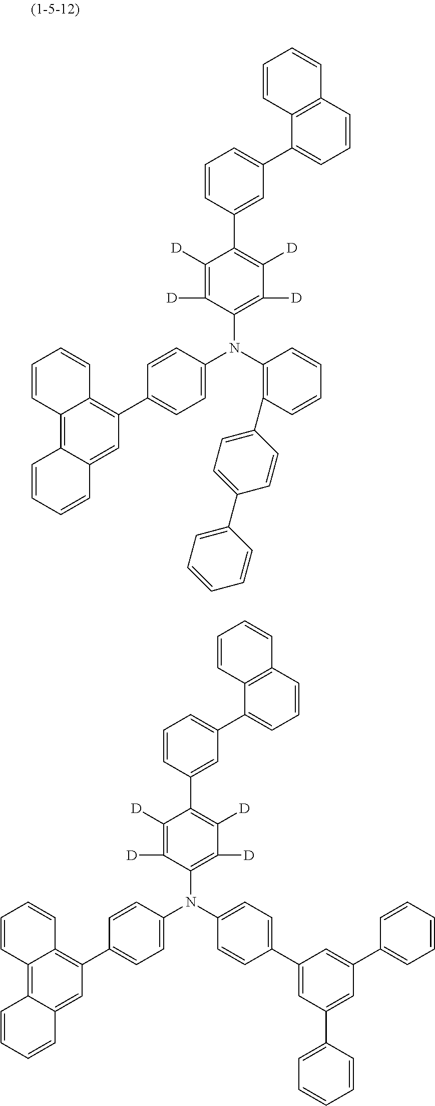

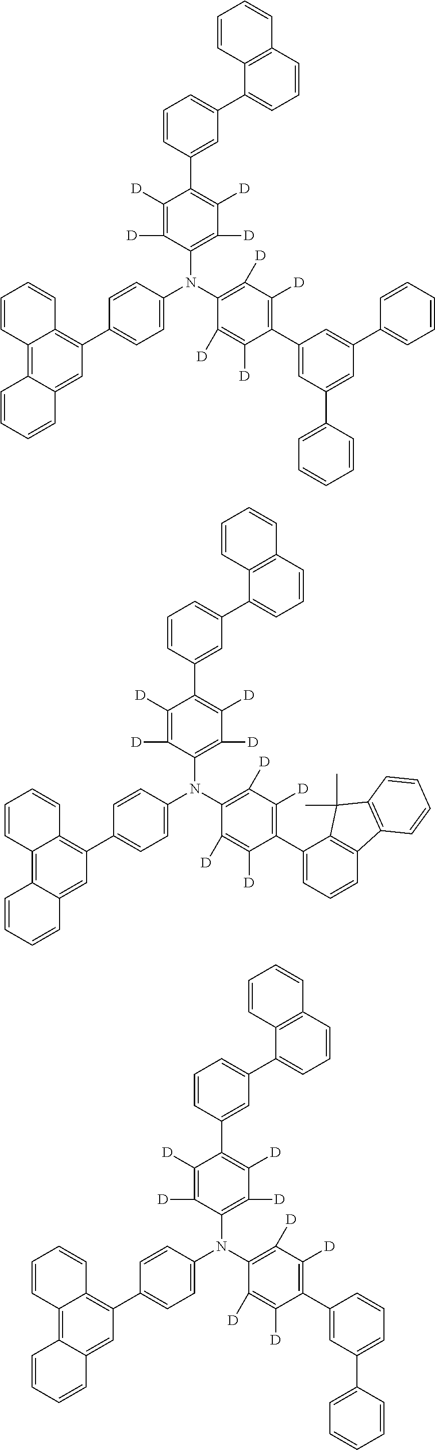 [SMILES: [2H]c1c([2H])c(N(c2ccc(-c3cc4ccccc4c4ccccc34)cc2)c2c([2H])c([2H])c(-c3cccc(-c4cccc5ccccc45)c3)c([2H])c2[2H])c([2H])c([2H])c1-c1cc(-c2ccccc2)cc(-c2ccccc2)c1 ; [2H]c1c([2H])c(N(c2ccc(-c3cc4ccccc4c4ccccc34)cc2)c2c([2H])c([2H])c(-c3cccc4c3C(C)(C)c3ccccc3-4)c([2H])c2[2H])c([2H])c([2H])c1-c1cccc(-c2cccc3ccccc23)c1 ; [2H]c1c([2H])c(N(c2ccc(-c3cc4ccccc4c4ccccc34)cc2)c2c([2H])c([2H])c(-c3cccc(-c4cccc5ccccc45)c3)c([2H])c2[2H])c([2H])c([2H])c1-c1cccc(-c2ccccc2)c1]

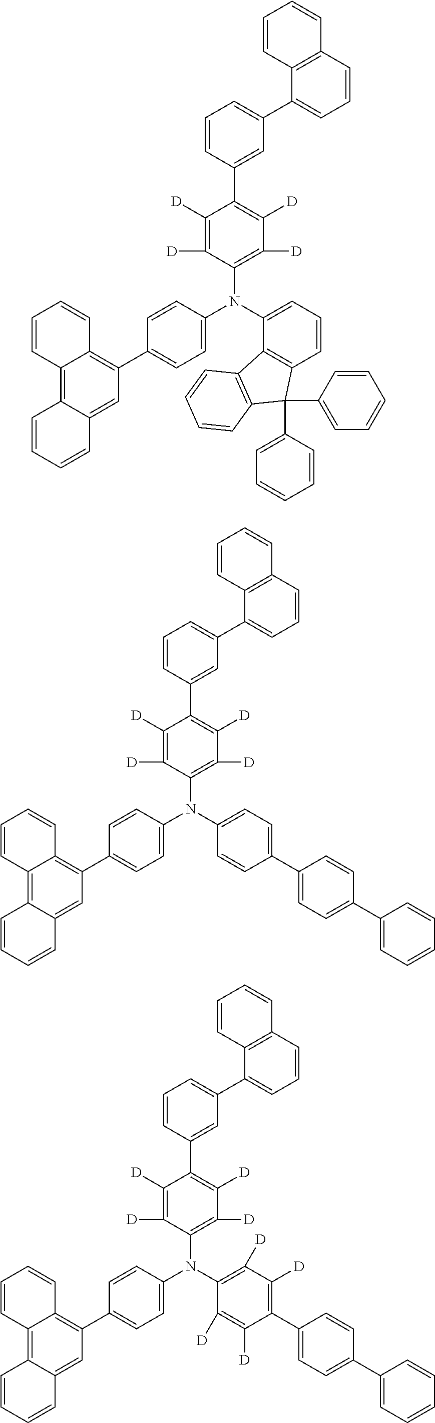 [SMILES: [2H]c1c([2H])c(N(c2ccc(-c3cc4ccccc4c4ccccc34)cc2)c2cccc3c2-c2ccccc2C3(c2ccccc2)c2ccccc2)c([2H])c([2H])c1-c1cccc(-c2cccc3ccccc23)c1 ; [2H]c1c([2H])c(N(c2ccc(-c3ccc(-c4ccccc4)cc3)cc2)c2ccc(-c3cc4ccccc4c4ccccc34)cc2)c([2H])c([2H])c1-c1cccc(-c2cccc3ccccc23)c1 ; [2H]c1c([2H])c(N(c2ccc(-c3cc4ccccc4c4ccccc34)cc2)c2c([2H])c([2H])c(-c3cccc(-c4cccc5ccccc45)c3)c([2H])c2[2H])c([2H])c([2H])c1-c1ccc(-c2ccccc2)cc1]

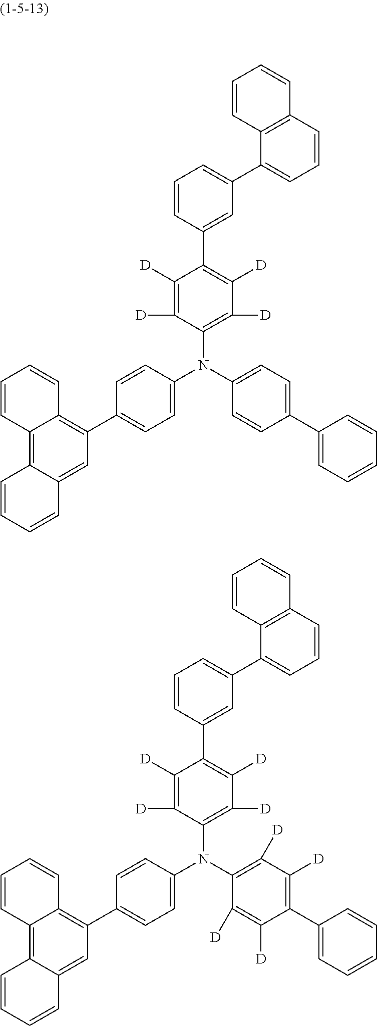 [SMILES: [2H]c1c([2H])c(N(c2ccc(-c3ccccc3)cc2)c2ccc(-c3cc4ccccc4c4ccccc34)cc2)c([2H])c([2H])c1-c1cccc(-c2cccc3ccccc23)c1 ; [2H]c1c([2H])c(N(c2ccc(-c3cc4ccccc4c4ccccc34)cc2)c2c([2H])c([2H])c(-c3cccc(-c4cccc5ccccc45)c3)c([2H])c2[2H])c([2H])c([2H])c1-c1ccccc1]

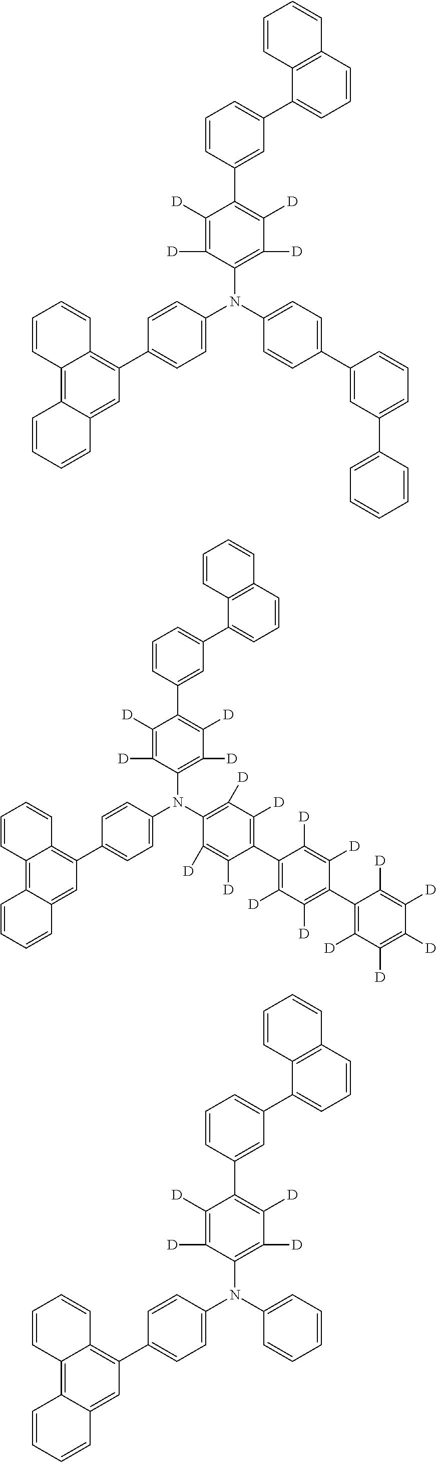 [SMILES: [2H]c1c([2H])c(N(c2ccc(-c3cccc(-c4ccccc4)c3)cc2)c2ccc(-c3cc4ccccc4c4ccccc34)cc2)c([2H])c([2H])c1-c1cccc(-c2cccc3ccccc23)c1 ; [2H]c1c([2H])c([2H])c(-c2c([2H])c([2H])c(-c3c([2H])c([2H])c(N(c4ccc(-c5cc6ccccc6c6ccccc56)cc4)c4c([2H])c([2H])c(-c5cccc(-c6cccc7ccccc67)c5)c([2H])c4[2H])c([2H])c3[2H])c([2H])c2[2H])c([2H])c1[2H] ; [2H]c1c([2H])c(N(c2ccccc2)c2ccc(-c3cc4ccccc4c4ccccc34)cc2)c([2H])c([2H])c1-c1cccc(-c2cccc3ccccc23)c1]

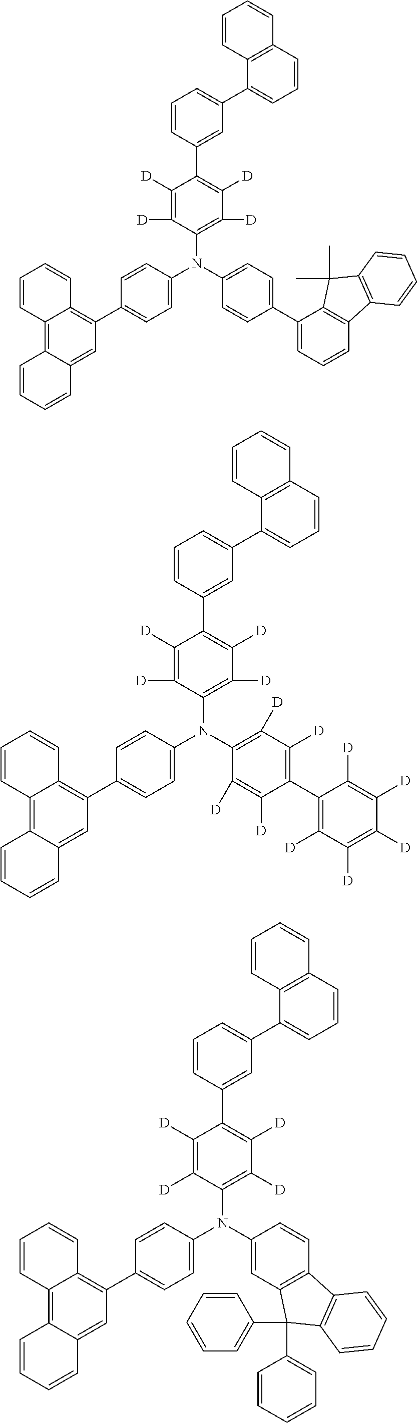 [SMILES: [2H]c1c([2H])c(N(c2ccc(-c3cccc4c3C(C)(C)c3ccccc3-4)cc2)c2ccc(-c3cc4ccccc4c4ccccc34)cc2)c([2H])c([2H])c1-c1cccc(-c2cccc3ccccc23)c1 ; [2H]c1c([2H])c([2H])c(-c2c([2H])c([2H])c(N(c3ccc(-c4cc5ccccc5c5ccccc45)cc3)c3c([2H])c([2H])c(-c4cccc(-c5cccc6ccccc56)c4)c([2H])c3[2H])c([2H])c2[2H])c([2H])c1[2H] ; [2H]c1c([2H])c(N(c2ccc(-c3cc4ccccc4c4ccccc34)cc2)c2ccc3c(c2)C(c2ccccc2)(c2ccccc2)c2ccccc2-3)c([2H])c([2H])c1-c1cccc(-c2cccc3ccccc23)c1]

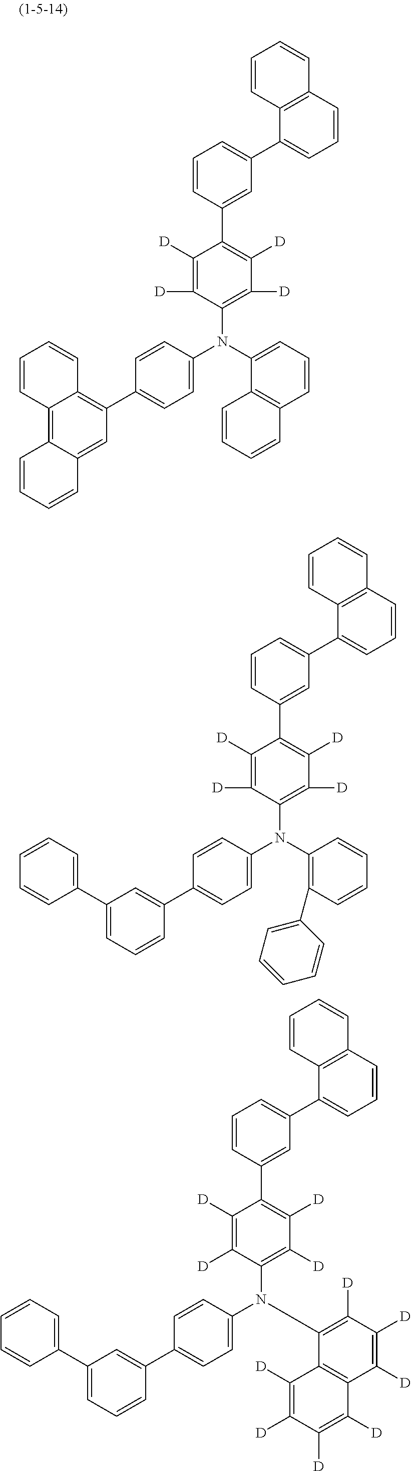

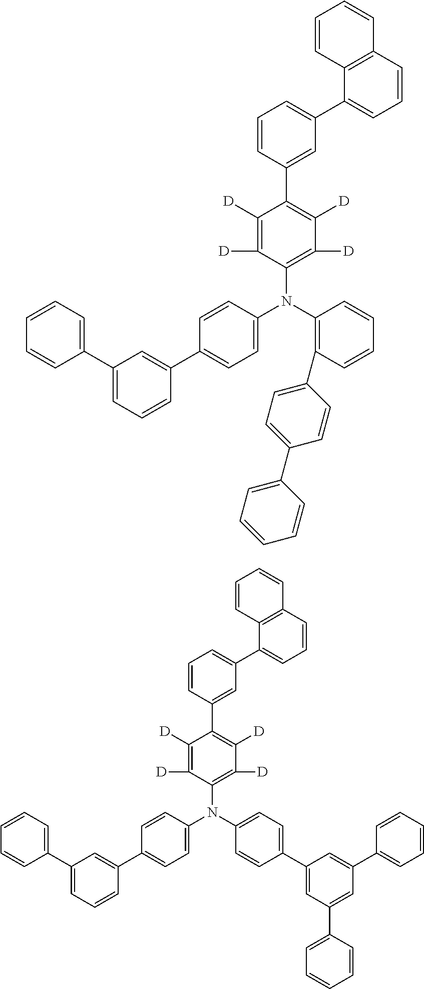

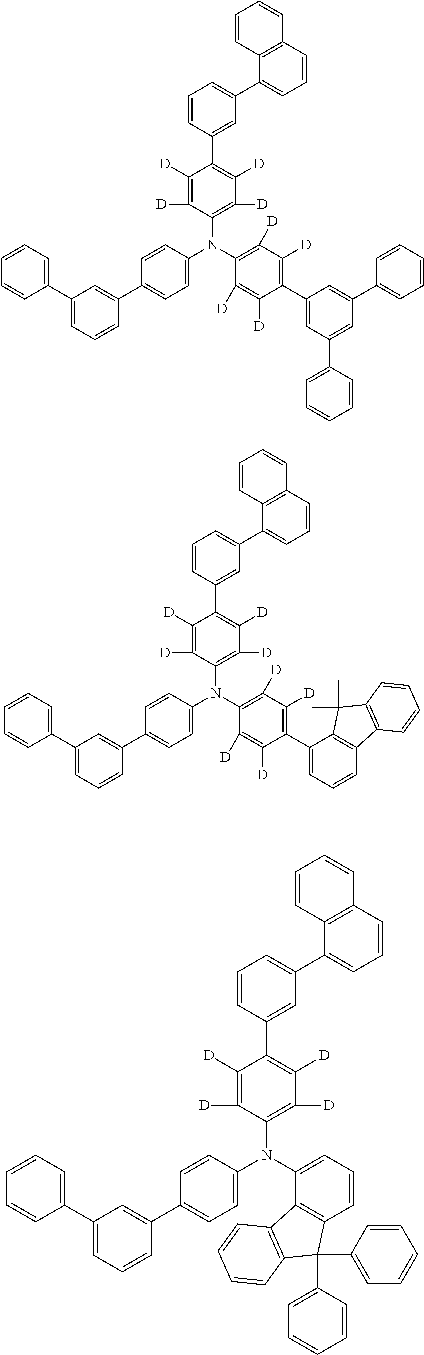 [SMILES: [2H]c1c([2H])c(N(c2ccc(-c3cccc(-c4ccccc4)c3)cc2)c2c([2H])c([2H])c(-c3cccc(-c4cccc5ccccc45)c3)c([2H])c2[2H])c([2H])c([2H])c1-c1cc(-c2ccccc2)cc(-c2ccccc2)c1 ; [2H]c1c([2H])c(N(c2ccc(-c3cccc(-c4ccccc4)c3)cc2)c2c([2H])c([2H])c(-c3cccc4c3C(C)(C)c3ccccc3-4)c([2H])c2[2H])c([2H])c([2H])c1-c1cccc(-c2cccc3ccccc23)c1 ; [2H]c1c([2H])c(N(c2ccc(-c3cccc(-c4ccccc4)c3)cc2)c2cccc3c2-c2ccccc2C3(c2ccccc2)c2ccccc2)c([2H])c([2H])c1-c1cccc(-c2cccc3ccccc23)c1]

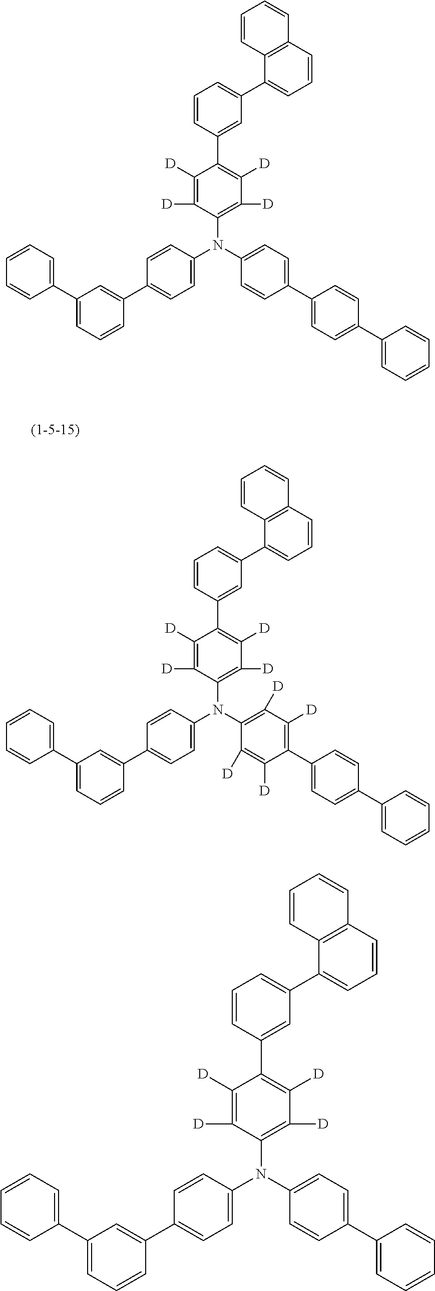 [SMILES: [2H]c1c([2H])c(N(c2ccc(-c3ccc(-c4ccccc4)cc3)cc2)c2ccc(-c3cccc(-c4ccccc4)c3)cc2)c([2H])c([2H])c1-c1cccc(-c2cccc3ccccc23)c1 ; [2H]c1c([2H])c(N(c2ccc(-c3cccc(-c4ccccc4)c3)cc2)c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2ccc(-c3ccccc3)cc2)c2ccc(-c3cccc(-c4ccccc4)c3)cc2)c([2H])c([2H])c1-c1cccc(-c2cccc3ccccc23)c1]

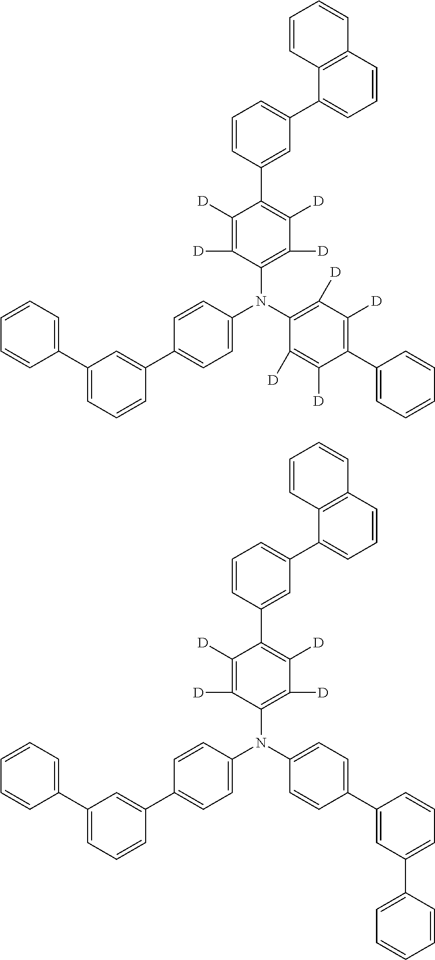

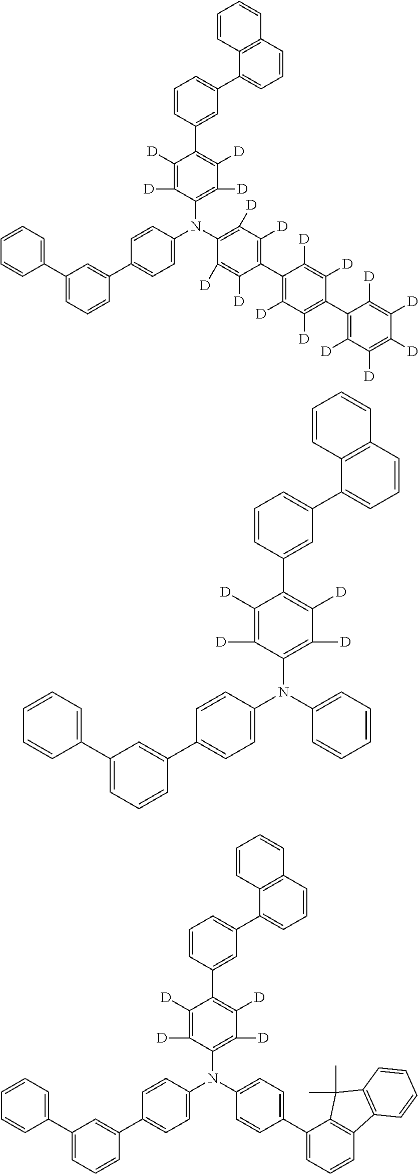

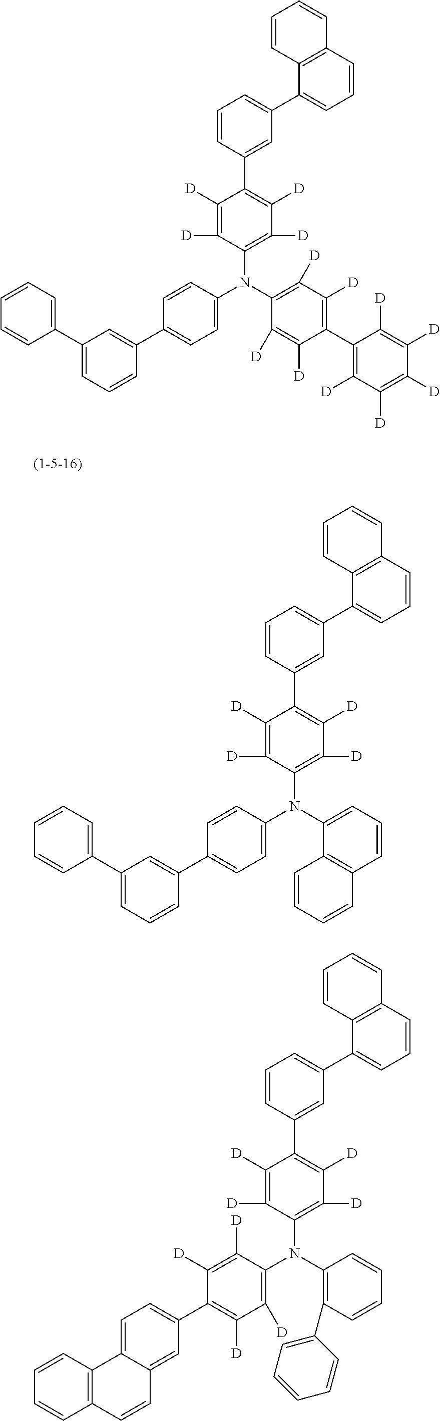

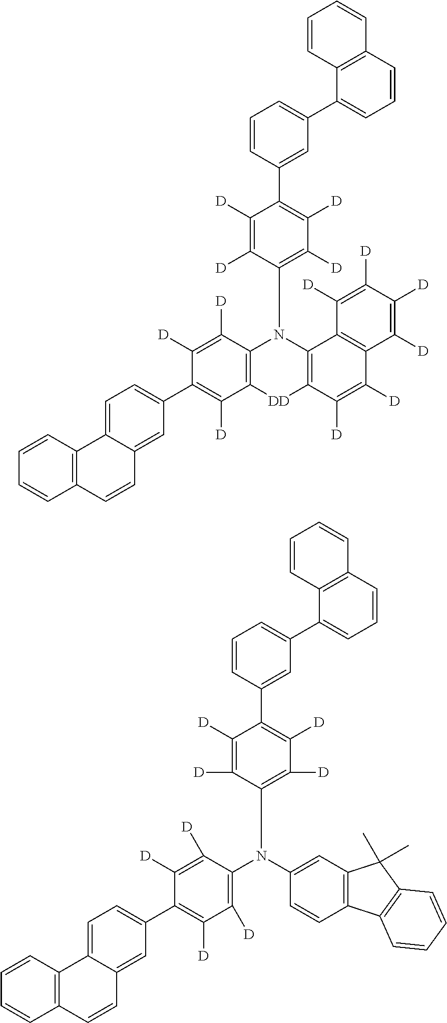

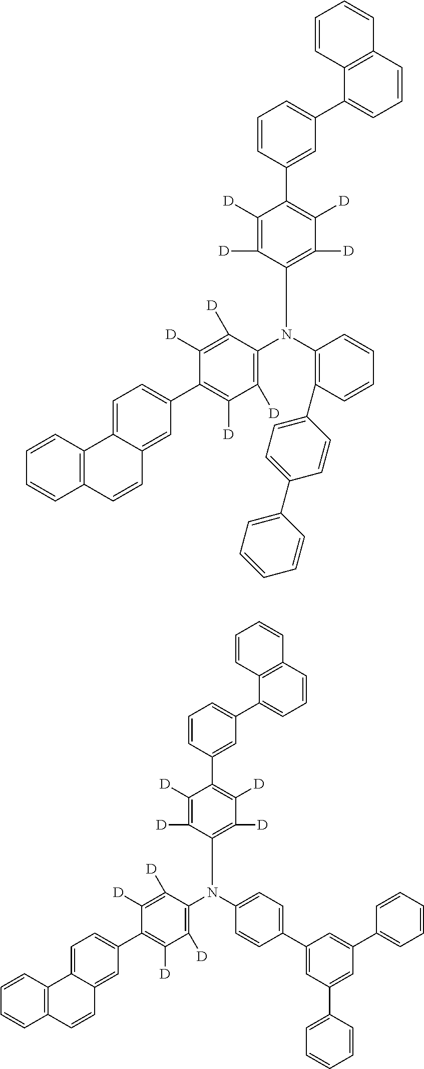 [SMILES: [2H]c1c([2H])c(-c2cccc(-c3cccc4ccccc34)c2)c([2H])c([2H])c1CN(c1ccccc1-c1ccc(-c2ccccc2)cc1)c1c([2H])c([2H])c(-c2ccc3c(ccc4ccccc43)c2)c([2H])c1[2H] ; [2H]c1c([2H])c(-c2cccc(-c3cccc4ccccc34)c2)c([2H])c([2H])c1CN(c1ccc(-c2cc(-c3ccccc3)cc(-c3ccccc3)c2)cc1)c1c([2H])c([2H])c(-c2ccc3c(ccc4ccccc43)c2)c([2H])c1[2H]]

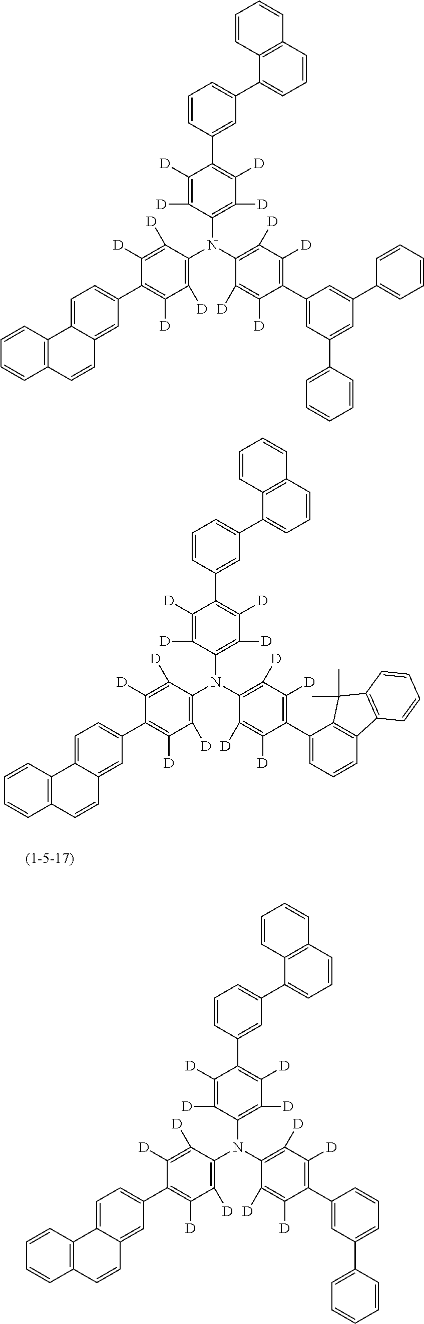

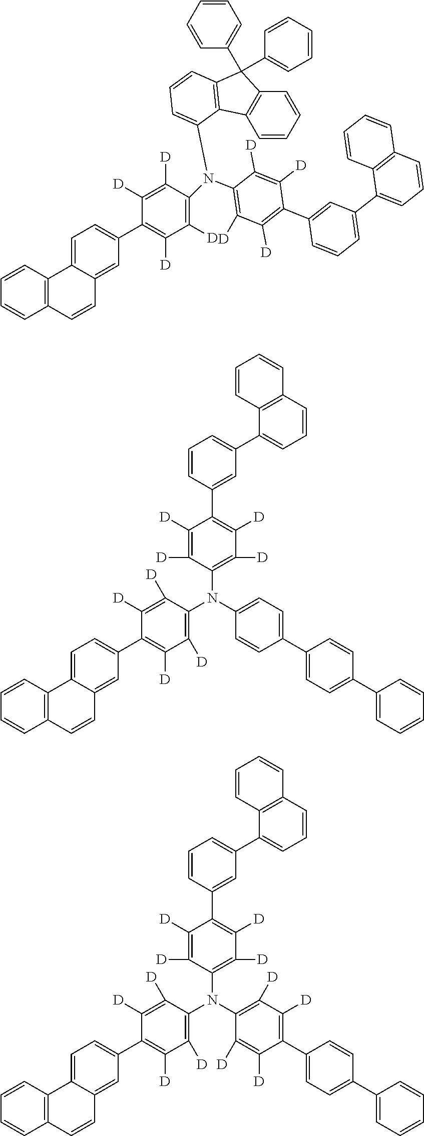 [SMILES: [2H]c1c([2H])c(N(Cc2cccc3c2-c2ccccc2C3(c2ccccc2)c2ccccc2)c2c([2H])c([2H])c(-c3ccc4c(ccc5ccccc54)c3)c([2H])c2[2H])c([2H])c([2H])c1-c1cccc(-c2cccc3ccccc23)c1 ; [2H]c1c([2H])c(N(c2ccc(-c3ccc(-c4ccccc4)cc3)cc2)c2c([2H])c([2H])c(-c3ccc4c(ccc5ccccc54)c3)c([2H])c2[2H])c([2H])c([2H])c1-c1cccc(-c2cccc3ccccc23)c1 ; [2H]c1c([2H])c(N(c2c([2H])c([2H])c(-c3cccc(-c4cccc5ccccc45)c3)c([2H])c2[2H])c2c([2H])c([2H])c(-c3ccc4c(ccc5ccccc54)c3)c([2H])c2[2H])c([2H])c([2H])c1-c1ccc(-c2ccccc2)cc1]

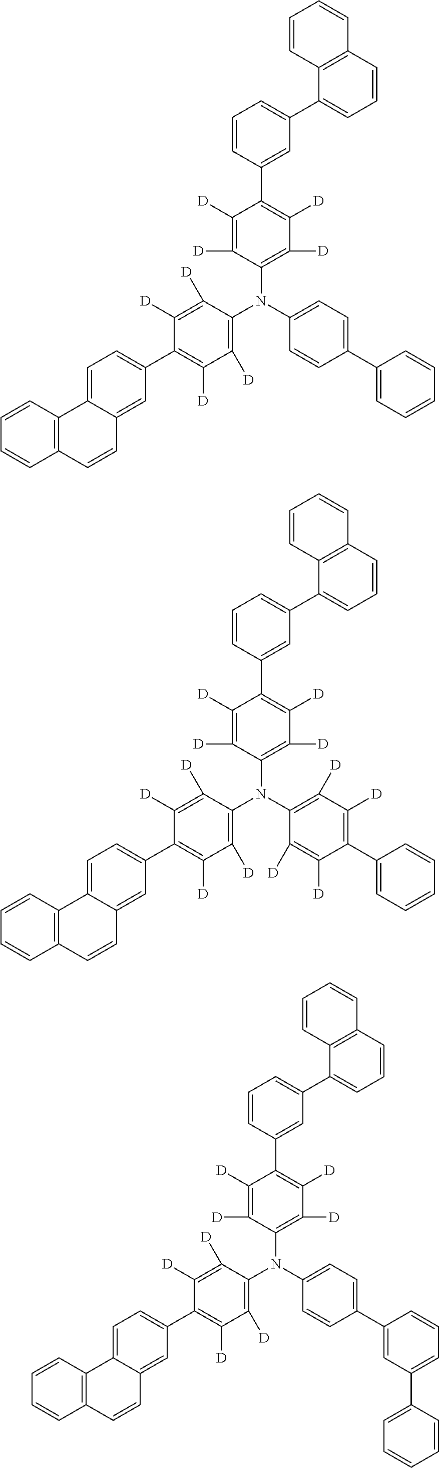 [SMILES: [2H]c1c([2H])c(N(c2ccc(-c3ccccc3)cc2)c2c([2H])c([2H])c(-c3ccc4c(ccc5ccccc54)c3)c([2H])c2[2H])c([2H])c([2H])c1-c1cccc(-c2cccc3ccccc23)c1 ; [2H]c1c([2H])c(N(c2c([2H])c([2H])c(-c3cccc(-c4cccc5ccccc45)c3)c([2H])c2[2H])c2c([2H])c([2H])c(-c3ccc4c(ccc5ccccc54)c3)c([2H])c2[2H])c([2H])c([2H])c1-c1ccccc1 ; [2H]c1c([2H])c(N(c2ccc(-c3cccc(-c4ccccc4)c3)cc2)c2c([2H])c([2H])c(-c3ccc4c(ccc5ccccc54)c3)c([2H])c2[2H])c([2H])c([2H])c1-c1cccc(-c2cccc3ccccc23)c1]

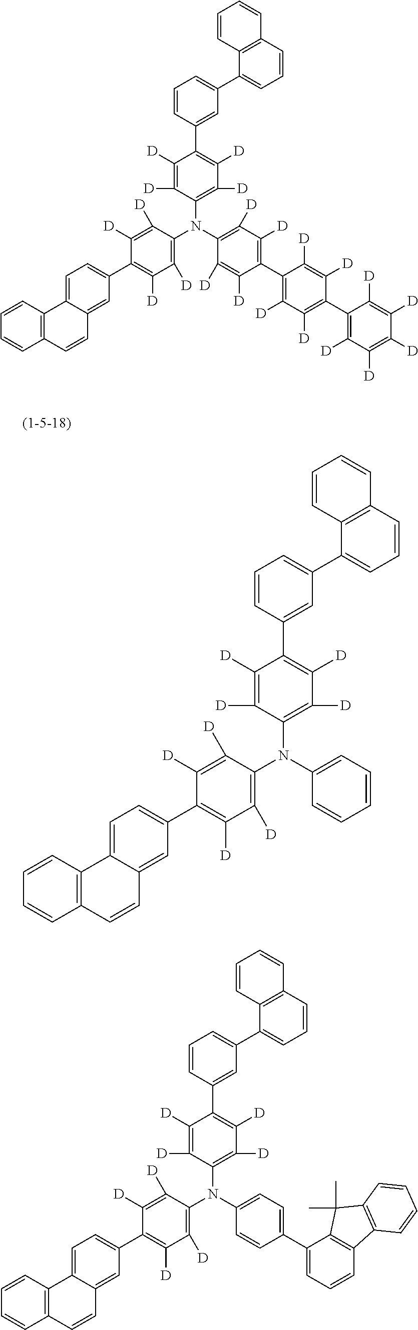

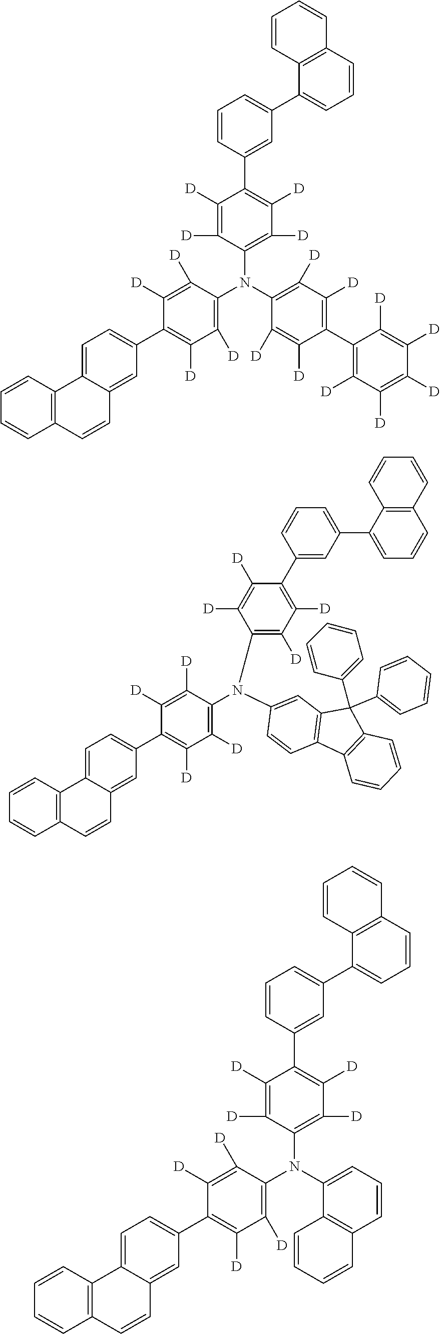

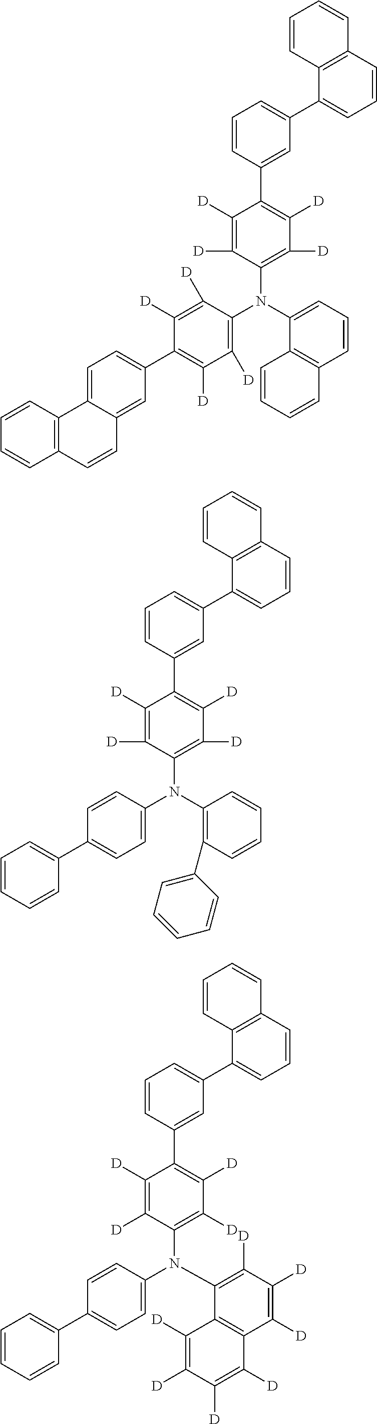

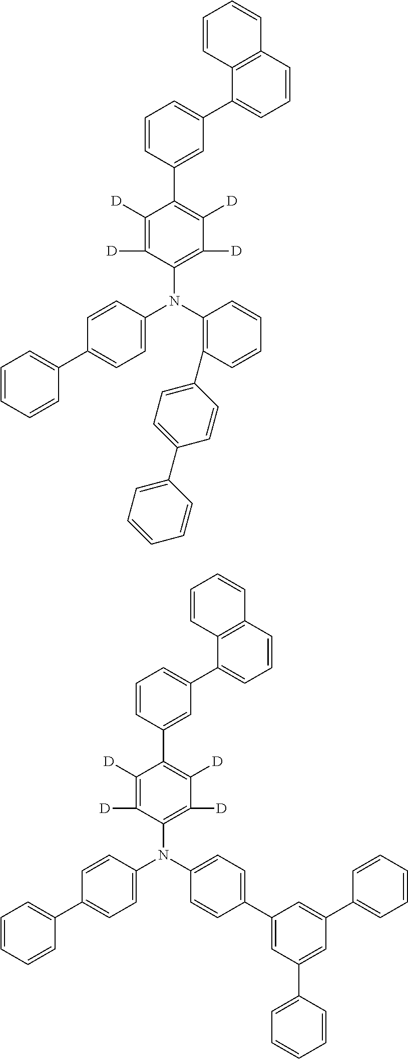 [SMILES: [2H]c1c([2H])c(N(c2ccc(-c3ccccc3)cc2)c2ccccc2-c2ccc(-c3ccccc3)cc2)c([2H])c([2H])c1-c1cccc(-c2cccc3ccccc23)c1 ; [2H]c1c([2H])c(N(c2ccc(-c3ccccc3)cc2)c2ccc(-c3cc(-c4ccccc4)cc(-c4ccccc4)c3)cc2)c([2H])c([2H])c1-c1cccc(-c2cccc3ccccc23)c1]

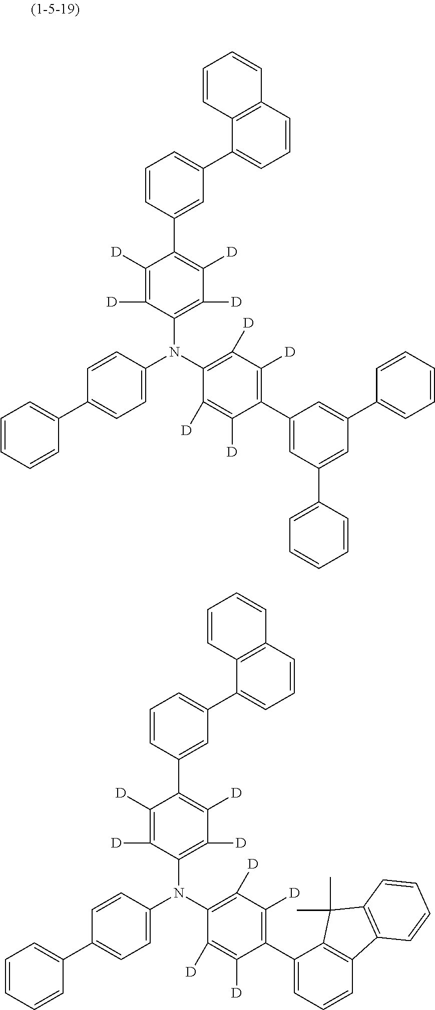 [SMILES: [2H]c1c([2H])c(N(c2ccc(-c3ccccc3)cc2)c2c([2H])c([2H])c(-c3cccc(-c4cccc5ccccc45)c3)c([2H])c2[2H])c([2H])c([2H])c1-c1cc(-c2ccccc2)cc(-c2ccccc2)c1 ; [2H]c1c([2H])c(N(c2ccc(-c3ccccc3)cc2)c2c([2H])c([2H])c(-c3cccc4c3C(C)(C)c3ccccc3-4)c([2H])c2[2H])c([2H])c([2H])c1-c1cccc(-c2cccc3ccccc23)c1]

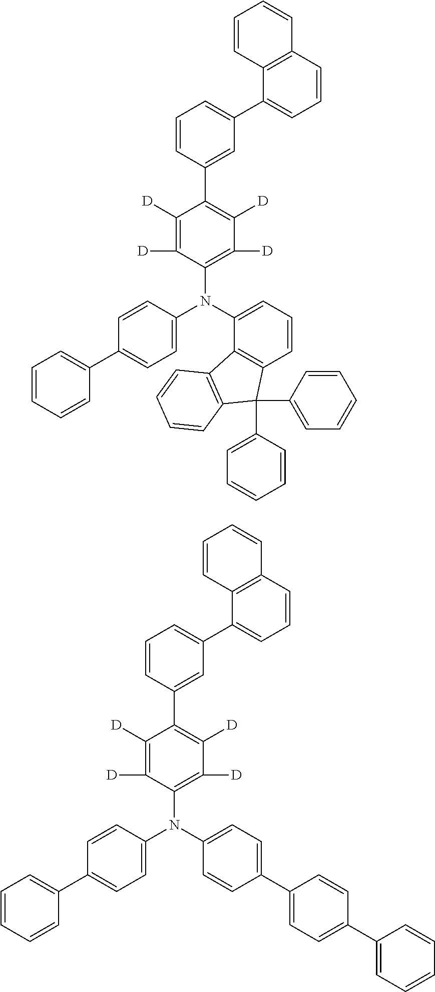

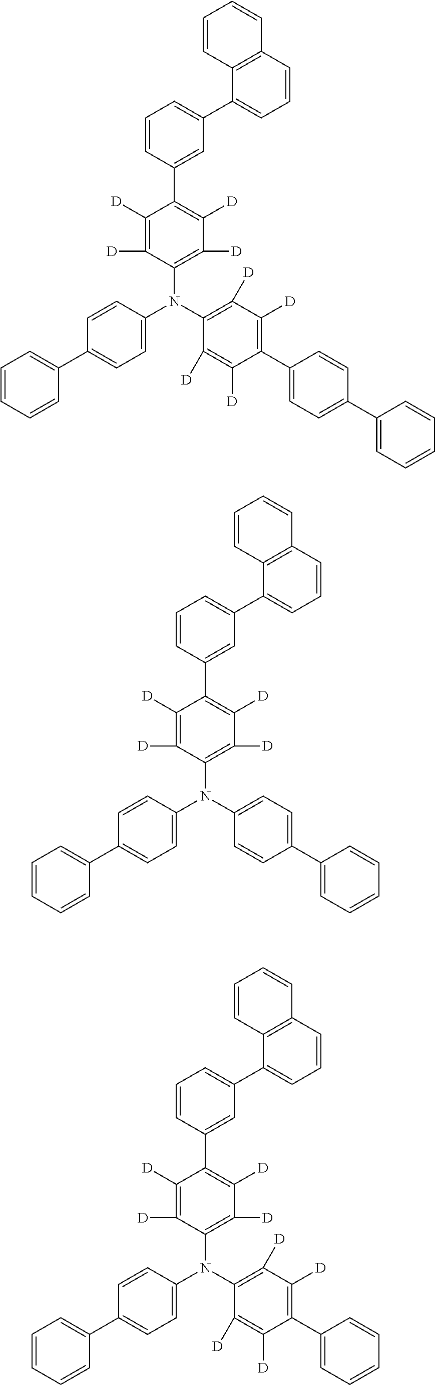 [SMILES: [2H]c1c([2H])c(N(c2ccc(-c3ccccc3)cc2)c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2ccc(-c3ccccc3)cc2)c2ccc(-c3ccccc3)cc2)c([2H])c([2H])c1-c1cccc(-c2cccc3ccccc23)c1 ; [2H]c1c([2H])c(N(c2ccc(-c3ccccc3)cc2)c2c([2H])c([2H])c(-c3cccc(-c4cccc5ccccc45)c3)c([2H])c2[2H])c([2H])c([2H])c1-c1ccccc1]

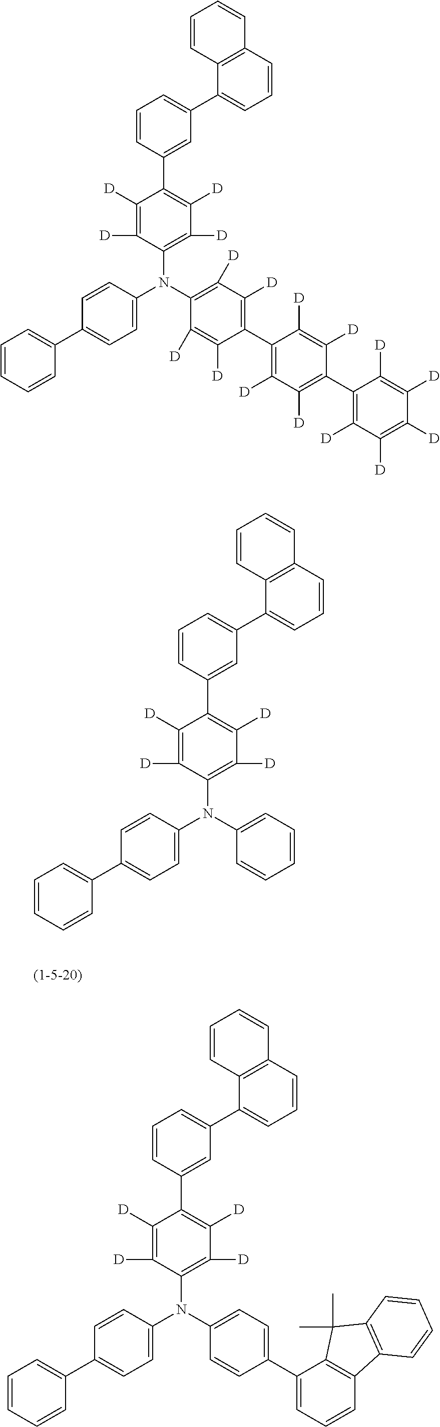 [SMILES: [2H]c1c([2H])c([2H])c(-c2c([2H])c([2H])c(-c3c([2H])c([2H])c(N(c4ccc(-c5ccccc5)cc4)c4c([2H])c([2H])c(-c5cccc(-c6cccc7ccccc67)c5)c([2H])c4[2H])c([2H])c3[2H])c([2H])c2[2H])c([2H])c1[2H] ; [2H]c1c([2H])c(N(c2ccccc2)c2ccc(-c3ccccc3)cc2)c([2H])c([2H])c1-c1cccc(-c2cccc3ccccc23)c1 ; [2H]c1c([2H])c(N(c2ccc(-c3ccccc3)cc2)c2ccc(-c3cccc4c3C(C)(C)c3ccccc3-4)cc2)c([2H])c([2H])c1-c1cccc(-c2cccc3ccccc23)c1]

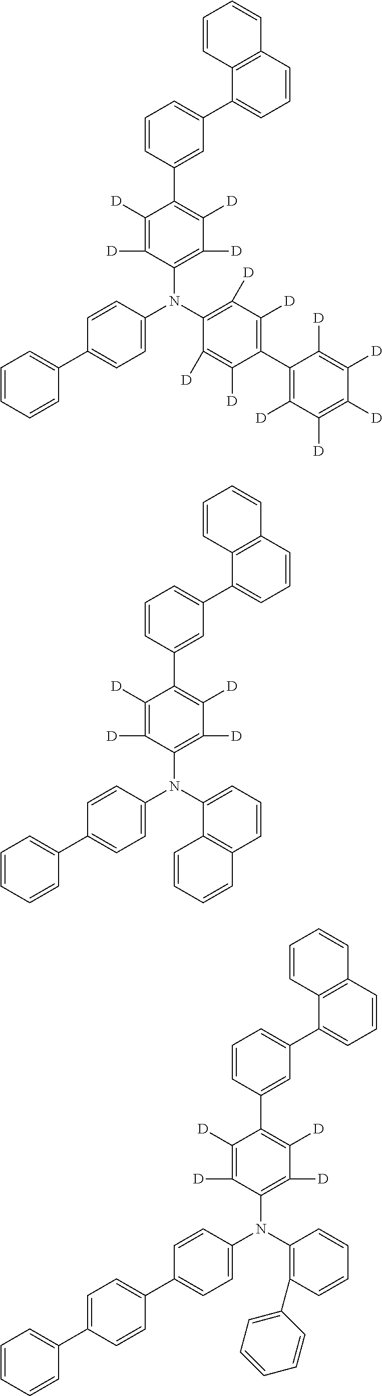 [SMILES: [2H]c1c([2H])c([2H])c(-c2c([2H])c([2H])c(N(c3ccc(-c4ccccc4)cc3)c3c([2H])c([2H])c(-c4cccc(-c5cccc6ccccc56)c4)c([2H])c3[2H])c([2H])c2[2H])c([2H])c1[2H] ; [2H]c1c([2H])c(N(c2ccc(-c3ccccc3)cc2)c2cccc3ccccc23)c([2H])c([2H])c1-c1cccc(-c2cccc3ccccc23)c1 ; [2H]c1c([2H])c(N(c2ccc(-c3ccc(-c4ccccc4)cc3)cc2)c2ccccc2-c2ccccc2)c([2H])c([2H])c1-c1cccc(-c2cccc3ccccc23)c1]

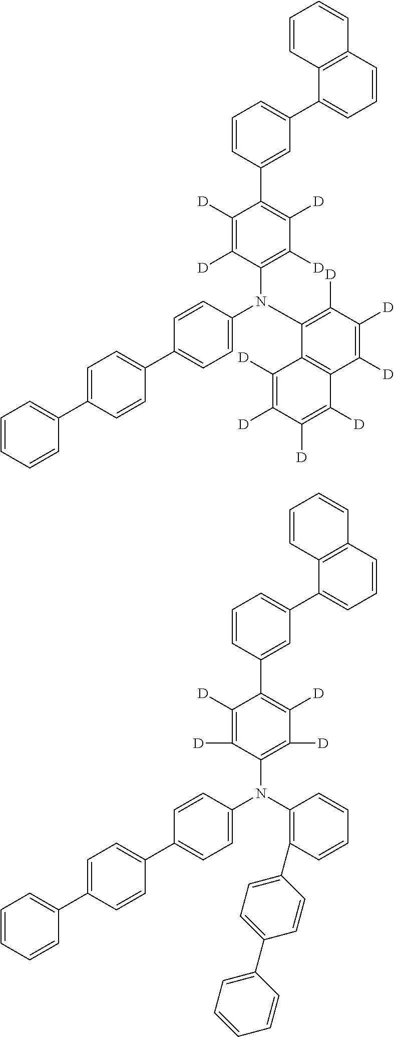

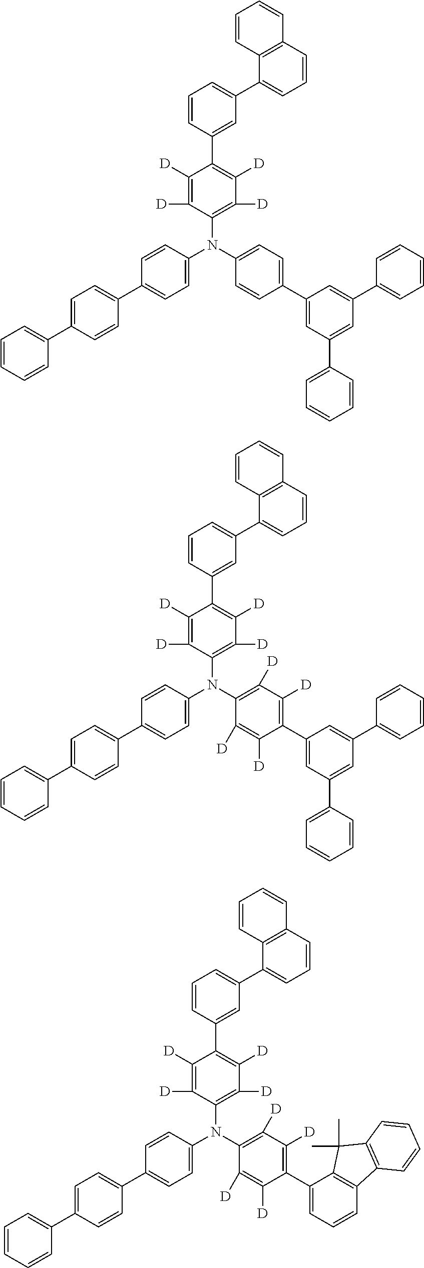 [SMILES: [2H]c1c([2H])c(N(c2ccc(-c3ccc(-c4ccccc4)cc3)cc2)c2ccc(-c3cc(-c4ccccc4)cc(-c4ccccc4)c3)cc2)c([2H])c([2H])c1-c1cccc(-c2cccc3ccccc23)c1 ; [2H]c1c([2H])c(N(c2ccc(-c3ccc(-c4ccccc4)cc3)cc2)c2c([2H])c([2H])c(-c3cccc(-c4cccc5ccccc45)c3)c([2H])c2[2H])c([2H])c([2H])c1-c1cc(-c2ccccc2)cc(-c2ccccc2)c1 ; [2H]c1c([2H])c(N(c2ccc(-c3ccc(-c4ccccc4)cc3)cc2)c2c([2H])c([2H])c(-c3cccc4c3C(C)(C)c3ccccc3-4)c([2H])c2[2H])c([2H])c([2H])c1-c1cccc(-c2cccc3ccccc23)c1]

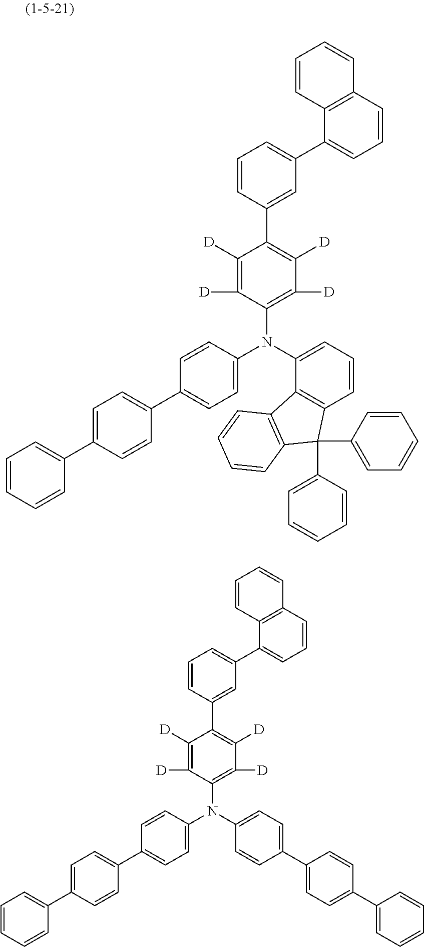 [SMILES: [2H]c1c([2H])c(N(c2ccc(-c3ccc(-c4ccccc4)cc3)cc2)c2cccc3c2-c2ccccc2C3(c2ccccc2)c2ccccc2)c([2H])c([2H])c1-c1cccc(-c2cccc3ccccc23)c1 ; [2H]c1c([2H])c(N(c2ccc(-c3ccc(-c4ccccc4)cc3)cc2)c2ccc(-c3ccc(-c4ccccc4)cc3)cc2)c([2H])c([2H])c1-c1cccc(-c2cccc3ccccc23)c1]

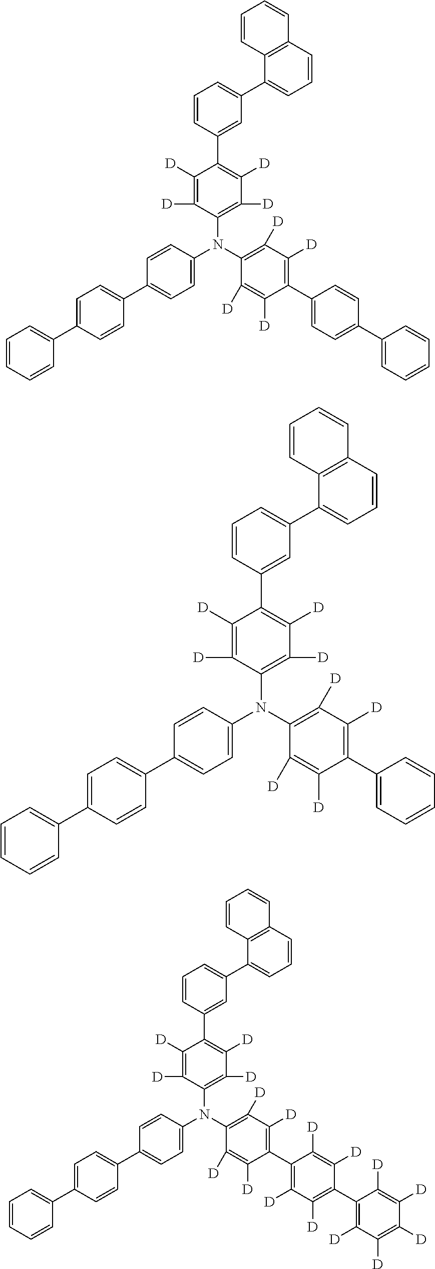 [SMILES: [2H]c1c([2H])c(N(c2ccc(-c3ccc(-c4ccccc4)cc3)cc2)c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2ccc(-c3ccc(-c4ccccc4)cc3)cc2)c2c([2H])c([2H])c(-c3cccc(-c4cccc5ccccc45)c3)c([2H])c2[2H])c([2H])c([2H])c1-c1ccccc1 ; [2H]c1c([2H])c([2H])c(-c2c([2H])c([2H])c(-c3c([2H])c([2H])c(N(c4ccc(-c5ccc(-c6ccccc6)cc5)cc4)c4c([2H])c([2H])c(-c5cccc(-c6cccc7ccccc67)c5)c([2H])c4[2H])c([2H])c3[2H])c([2H])c2[2H])c([2H])c1[2H]]

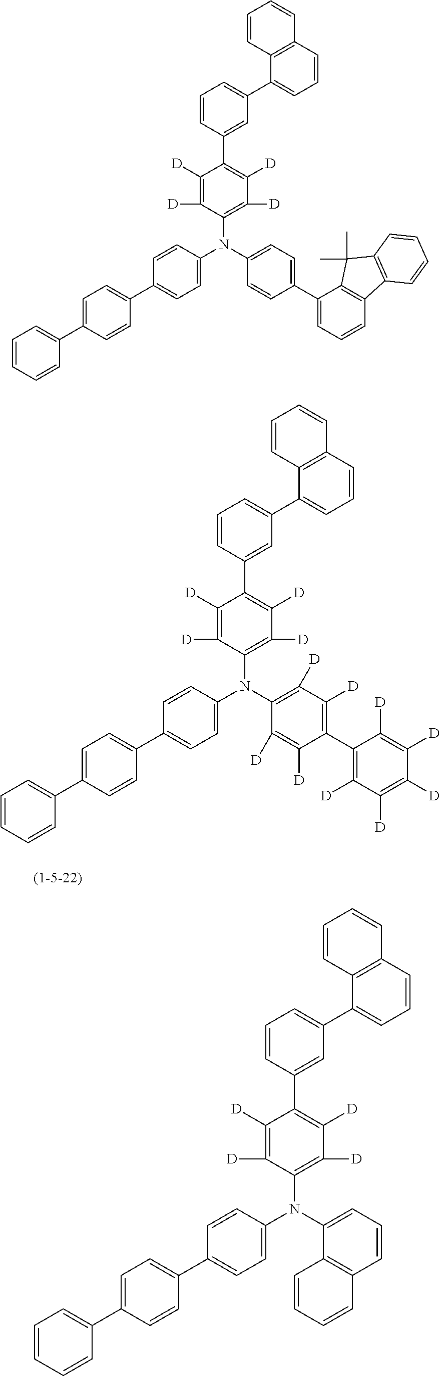 [SMILES: [2H]c1c([2H])c(N(c2ccc(-c3ccc(-c4ccccc4)cc3)cc2)c2ccc(-c3cccc4c3C(C)(C)c3ccccc3-4)cc2)c([2H])c([2H])c1-c1cccc(-c2cccc3ccccc23)c1 ; [2H]c1c([2H])c([2H])c(-c2c([2H])c([2H])c(N(c3ccc(-c4ccc(-c5ccccc5)cc4)cc3)c3c([2H])c([2H])c(-c4cccc(-c5cccc6ccccc56)c4)c([2H])c3[2H])c([2H])c2[2H])c([2H])c1[2H] ; [2H]c1c([2H])c(N(c2ccc(-c3ccc(-c4ccccc4)cc3)cc2)c2cccc3ccccc23)c([2H])c([2H])c1-c1cccc(-c2cccc3ccccc23)c1]

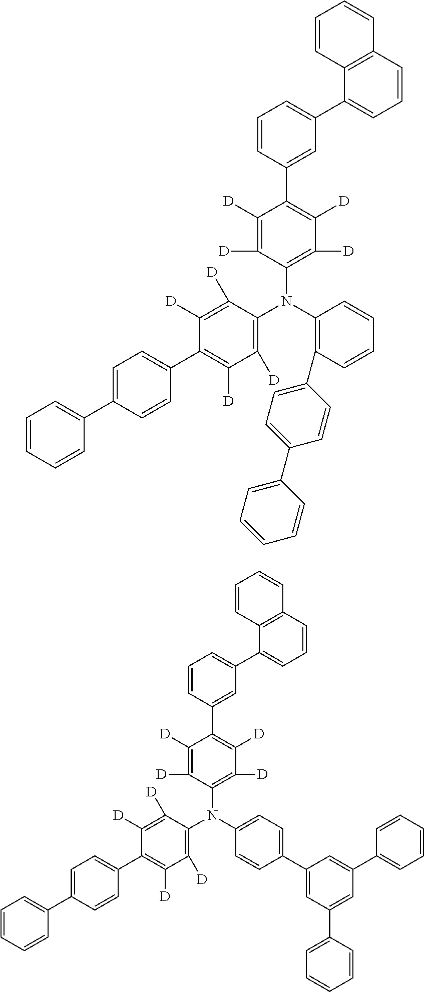

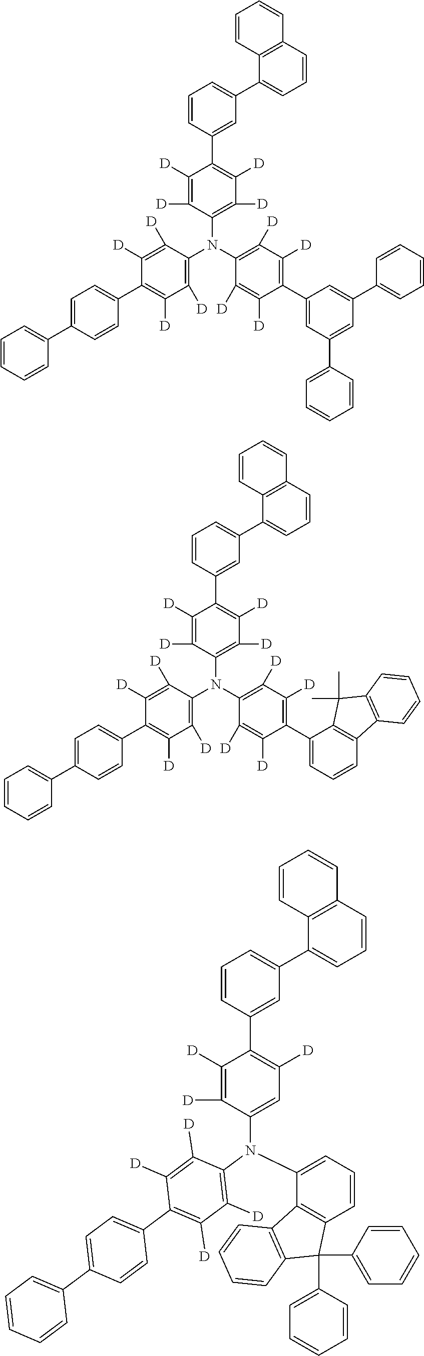 [SMILES: [2H]c1c([2H])c(N(c2c([2H])c([2H])c(-c3cc(-c4ccccc4)cc(-c4ccccc4)c3)c([2H])c2[2H])c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2c([2H])c([2H])c(-c3cccc(-c4cccc5ccccc45)c3)c([2H])c2[2H])c2c([2H])c([2H])c(-c3cccc4c3C(C)(C)c3ccccc3-4)c([2H])c2[2H])c([2H])c([2H])c1-c1ccc(-c2ccccc2)cc1 ; [2H]c1cc(N(c2cccc3c2-c2ccccc2C3(c2ccccc2)c2ccccc2)c2c([2H])c([2H])c(-c3ccc(-c4ccccc4)cc3)c([2H])c2[2H])c([2H])c([2H])c1-c1cccc(-c2cccc3ccccc23)c1]

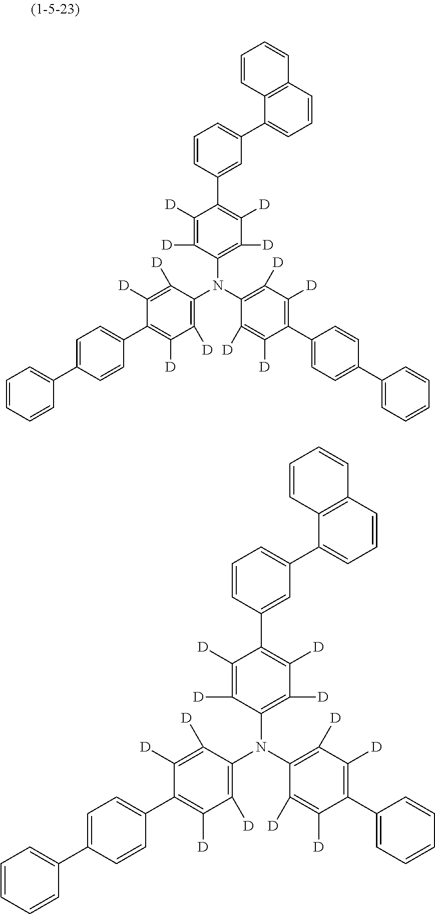 [SMILES: [2H]c1c([2H])c(N(c2c([2H])c([2H])c(-c3ccc(-c4ccccc4)cc3)c([2H])c2[2H])c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2c([2H])c([2H])c(-c3ccc(-c4ccccc4)cc3)c([2H])c2[2H])c2c([2H])c([2H])c(-c3cccc(-c4cccc5ccccc45)c3)c([2H])c2[2H])c([2H])c([2H])c1-c1ccccc1]

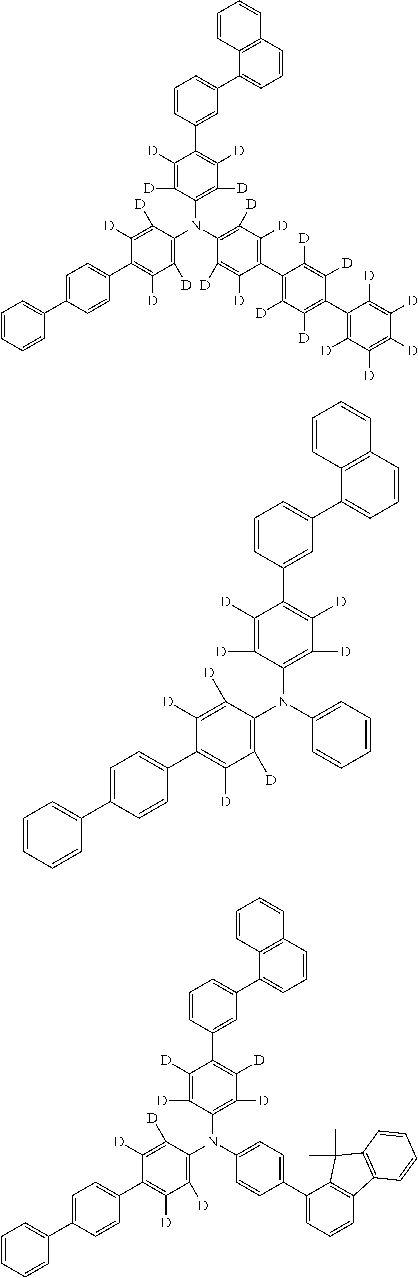 [SMILES: [2H]c1c([2H])c([2H])c(-c2c([2H])c([2H])c(-c3c([2H])c([2H])c(N(c4c([2H])c([2H])c(-c5ccc(-c6ccccc6)cc5)c([2H])c4[2H])c4c([2H])c([2H])c(-c5cccc(-c6cccc7ccccc67)c5)c([2H])c4[2H])c([2H])c3[2H])c([2H])c2[2H])c([2H])c1[2H] ; [2H]c1c([2H])c(N(c2ccccc2)c2c([2H])c([2H])c(-c3cccc(-c4cccc5ccccc45)c3)c([2H])c2[2H])c([2H])c([2H])c1-c1ccc(-c2ccccc2)cc1 ; [2H]c1c([2H])c(N(c2ccc(-c3cccc4c3C(C)(C)c3ccccc3-4)cc2)c2c([2H])c([2H])c(-c3cccc(-c4cccc5ccccc45)c3)c([2H])c2[2H])c([2H])c([2H])c1-c1ccc(-c2ccccc2)cc1]

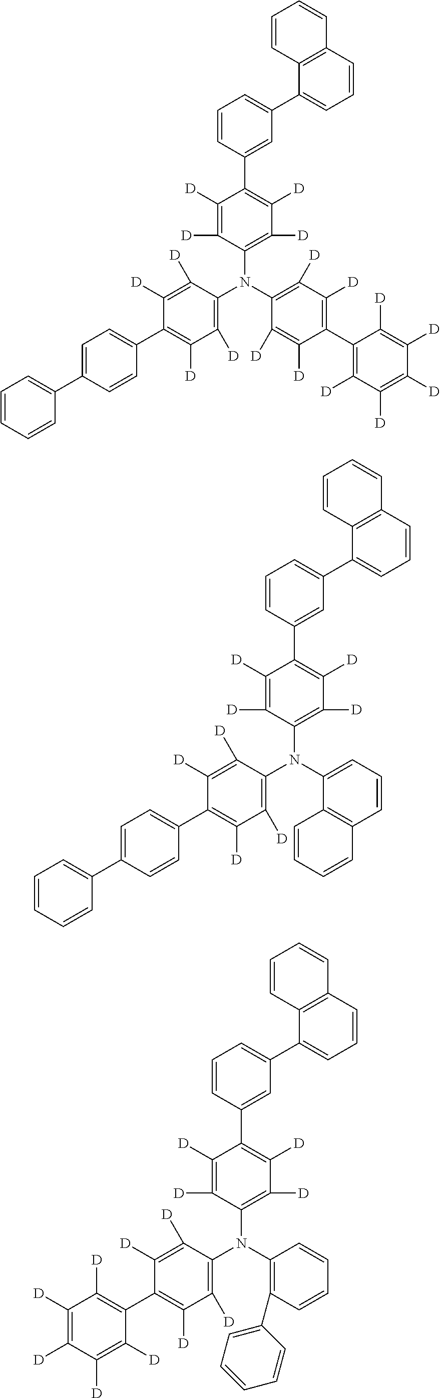

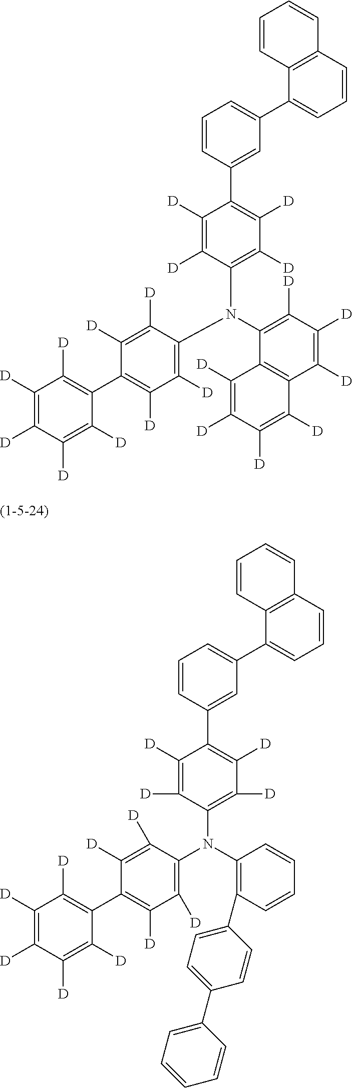 [SMILES: [2H]c1c([2H])c([2H])c(-c2c([2H])c([2H])c(N(c3c([2H])c([2H])c(-c4cccc(-c5cccc6ccccc56)c4)c([2H])c3[2H])c3c([2H])c([2H])c([2H])c4c([2H])c([2H])c([2H])c([2H])c34)c([2H])c2[2H])c([2H])c1[2H] ; [2H]c1c([2H])c([2H])c(-c2c([2H])c([2H])c(N(c3ccccc3-c3ccc(-c4ccccc4)cc3)c3c([2H])c([2H])c(-c4cccc(-c5cccc6ccccc56)c4)c([2H])c3[2H])c([2H])c2[2H])c([2H])c1[2H]]

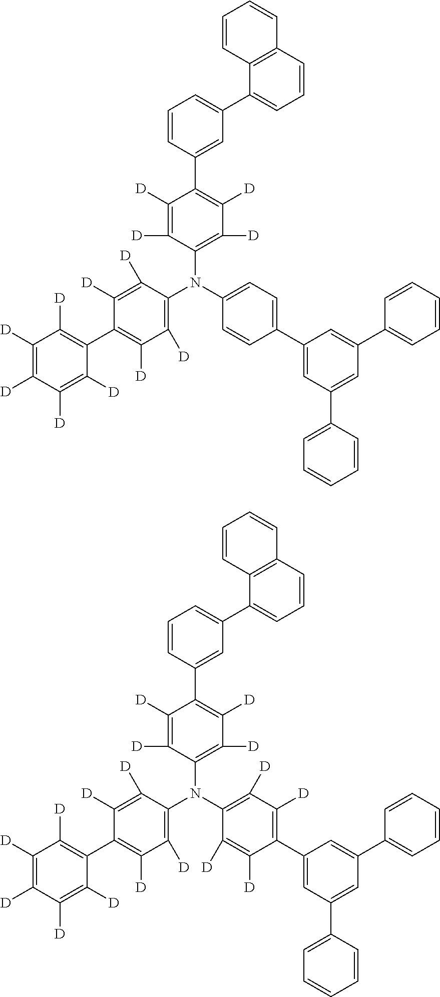 [SMILES: [2H]c1c([2H])c([2H])c(-c2c([2H])c([2H])c(N(c3ccc(-c4cc(-c5ccccc5)cc(-c5ccccc5)c4)cc3)c3c([2H])c([2H])c(-c4cccc(-c5cccc6ccccc56)c4)c([2H])c3[2H])c([2H])c2[2H])c([2H])c1[2H] ; [2H]c1c([2H])c([2H])c(-c2c([2H])c([2H])c(N(c3c([2H])c([2H])c(-c4cc(-c5ccccc5)cc(-c5ccccc5)c4)c([2H])c3[2H])c3c([2H])c([2H])c(-c4cccc(-c5cccc6ccccc56)c4)c([2H])c3[2H])c([2H])c2[2H])c([2H])c1[2H]]

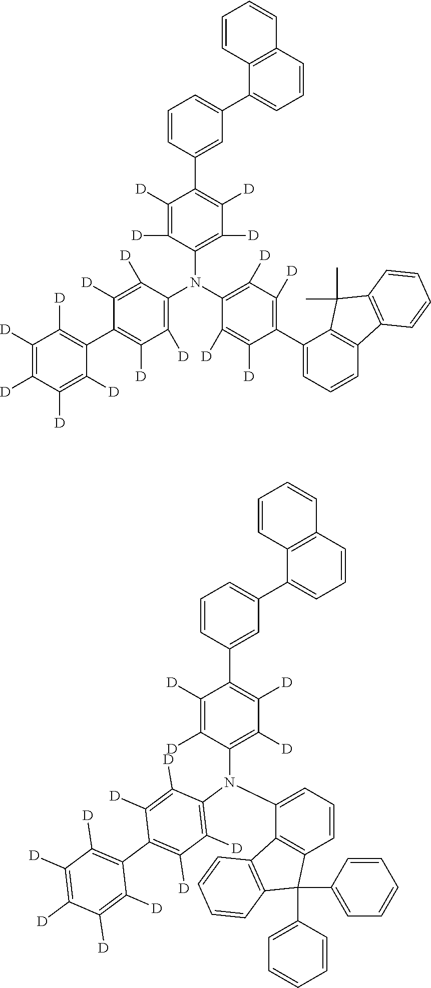 [SMILES: [2H]c1c([2H])c([2H])c(-c2c([2H])c([2H])c(N(c3c([2H])c([2H])c(-c4cccc(-c5cccc6ccccc56)c4)c([2H])c3[2H])c3c([2H])c([2H])c(-c4cccc5c4C(C)(C)c4ccccc4-5)c([2H])c3[2H])c([2H])c2[2H])c([2H])c1[2H] ; [2H]c1c([2H])c([2H])c(-c2c([2H])c([2H])c(N(c3cccc4c3-c3ccccc3C4(c3ccccc3)c3ccccc3)c3c([2H])c([2H])c(-c4cccc(-c5cccc6ccccc56)c4)c([2H])c3[2H])c([2H])c2[2H])c([2H])c1[2H]]

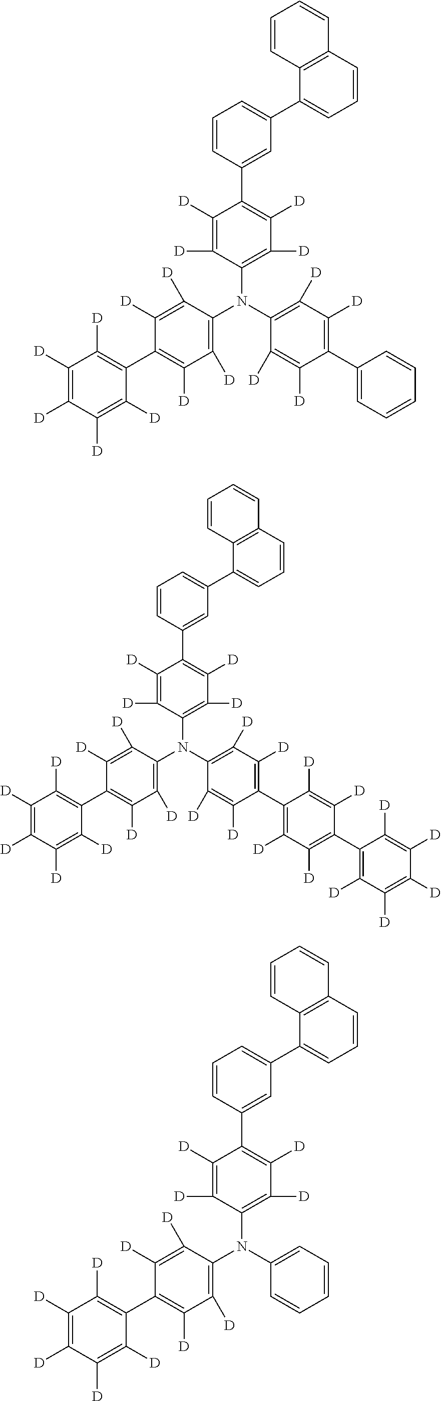

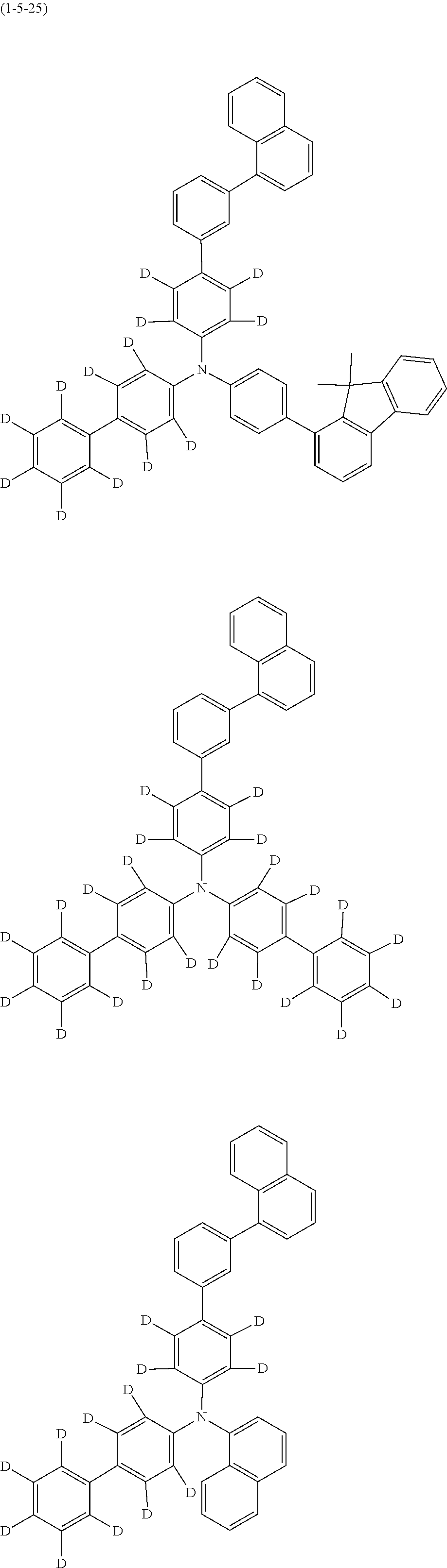

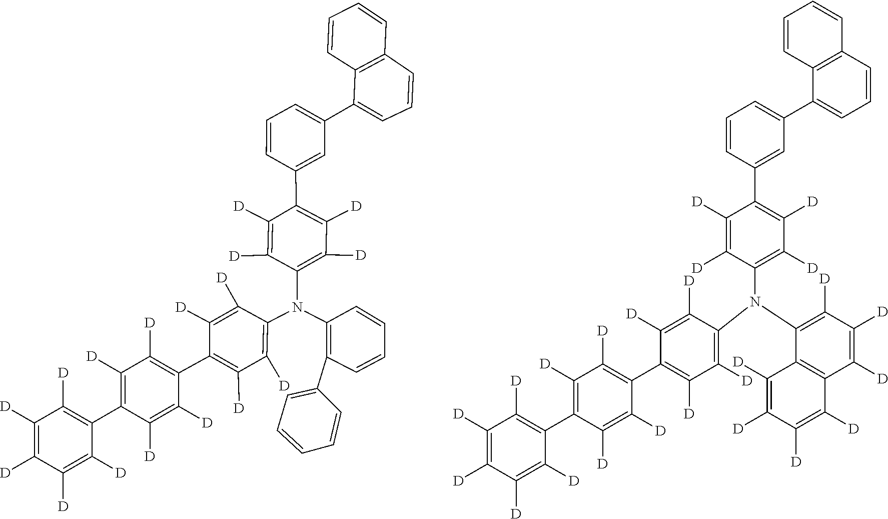 [SMILES: [2H]c1c([2H])c([2H])c(-c2c([2H])c([2H])c(-c3c([2H])c([2H])c(N(c4ccccc4-c4ccccc4)c4c([2H])c([2H])c(-c5cccc(-c6cccc7ccccc67)c5)c([2H])c4[2H])c([2H])c3[2H])c([2H])c2[2H])c([2H])c1[2H] ; [2H]c1c([2H])c([2H])c(-c2c([2H])c([2H])c(-c3c([2H])c([2H])c(N(c4c([2H])c([2H])c(-c5cccc(-c6cccc7ccccc67)c5)c([2H])c4[2H])c4c([2H])c([2H])c([2H])c5c([2H])c([2H])c([2H])c([2H])c45)c([2H])c3[2H])c([2H])c2[2H])c([2H])c1[2H]]

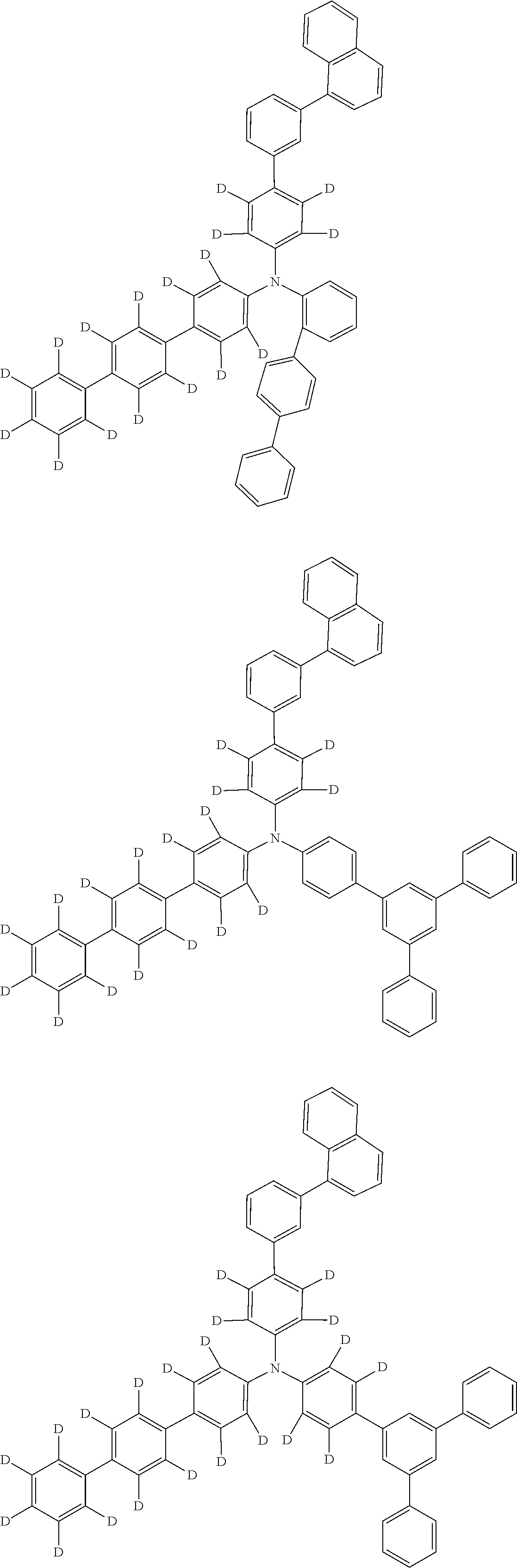

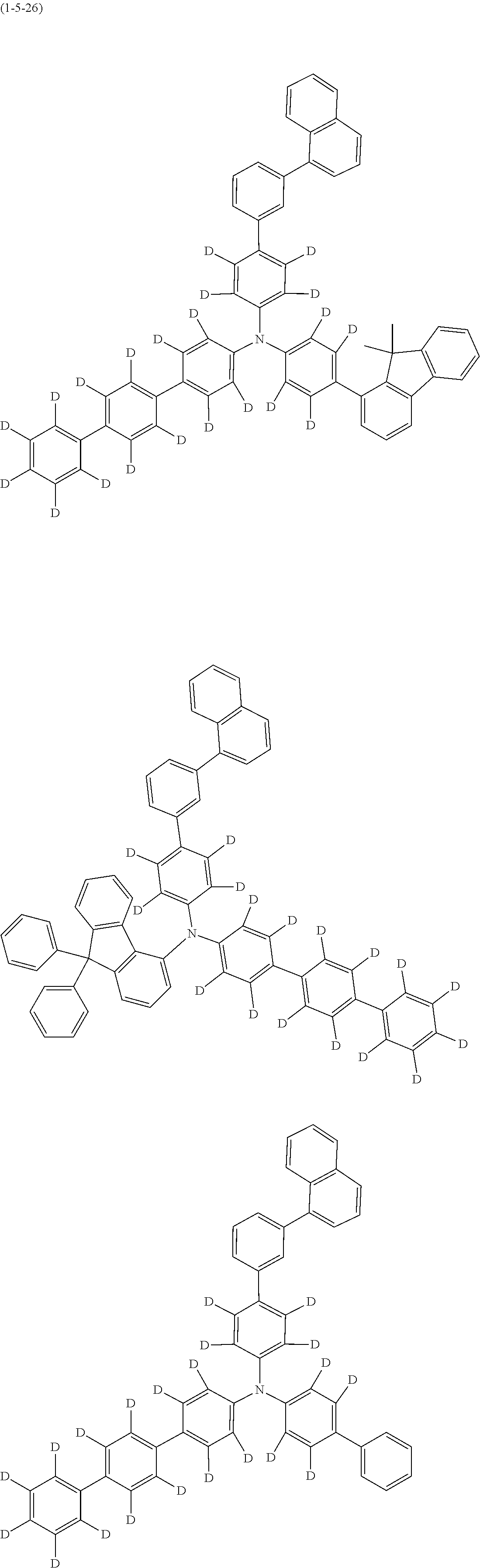

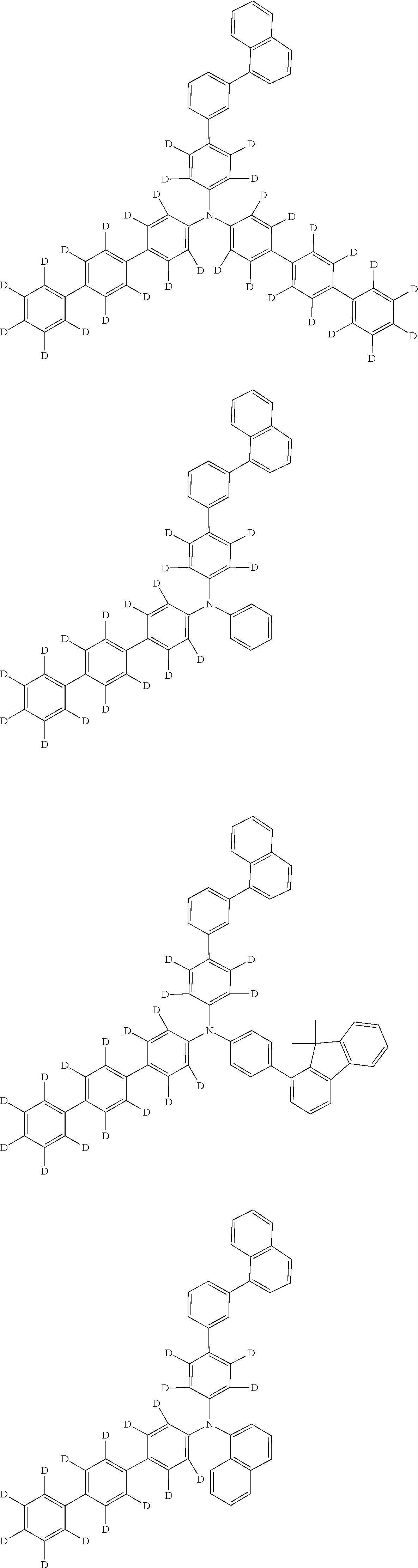

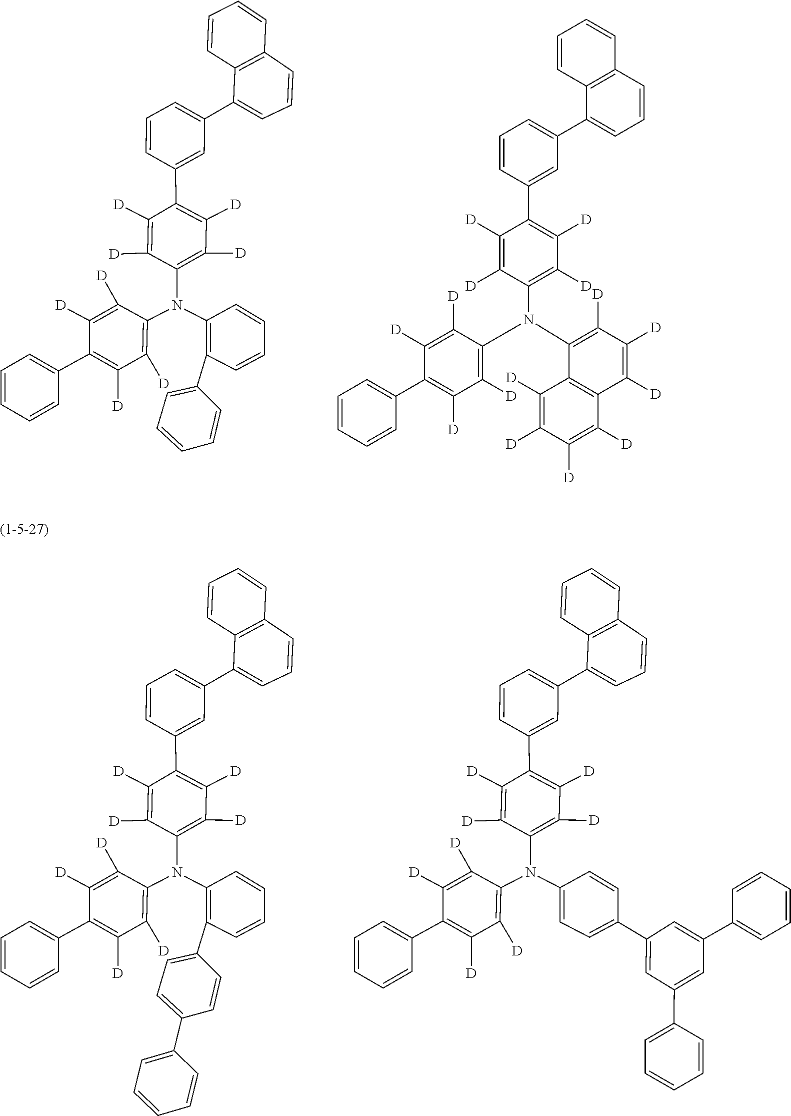

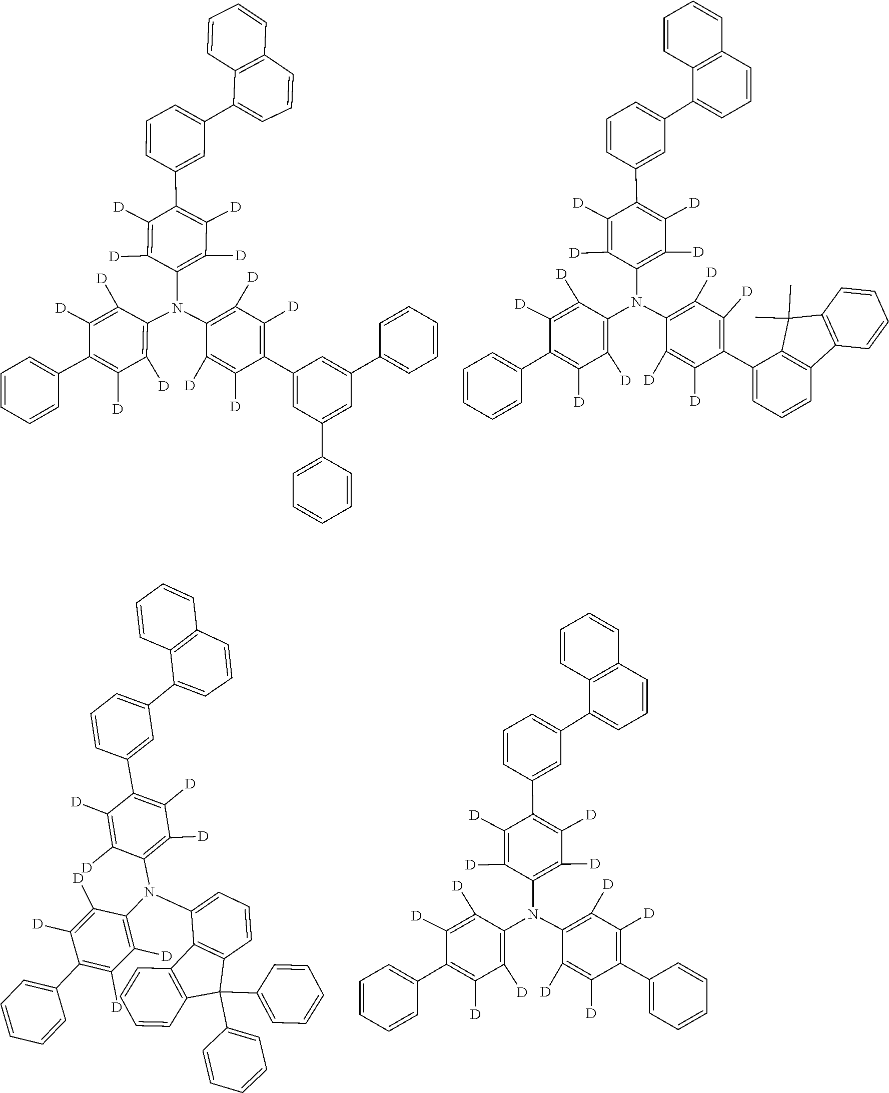

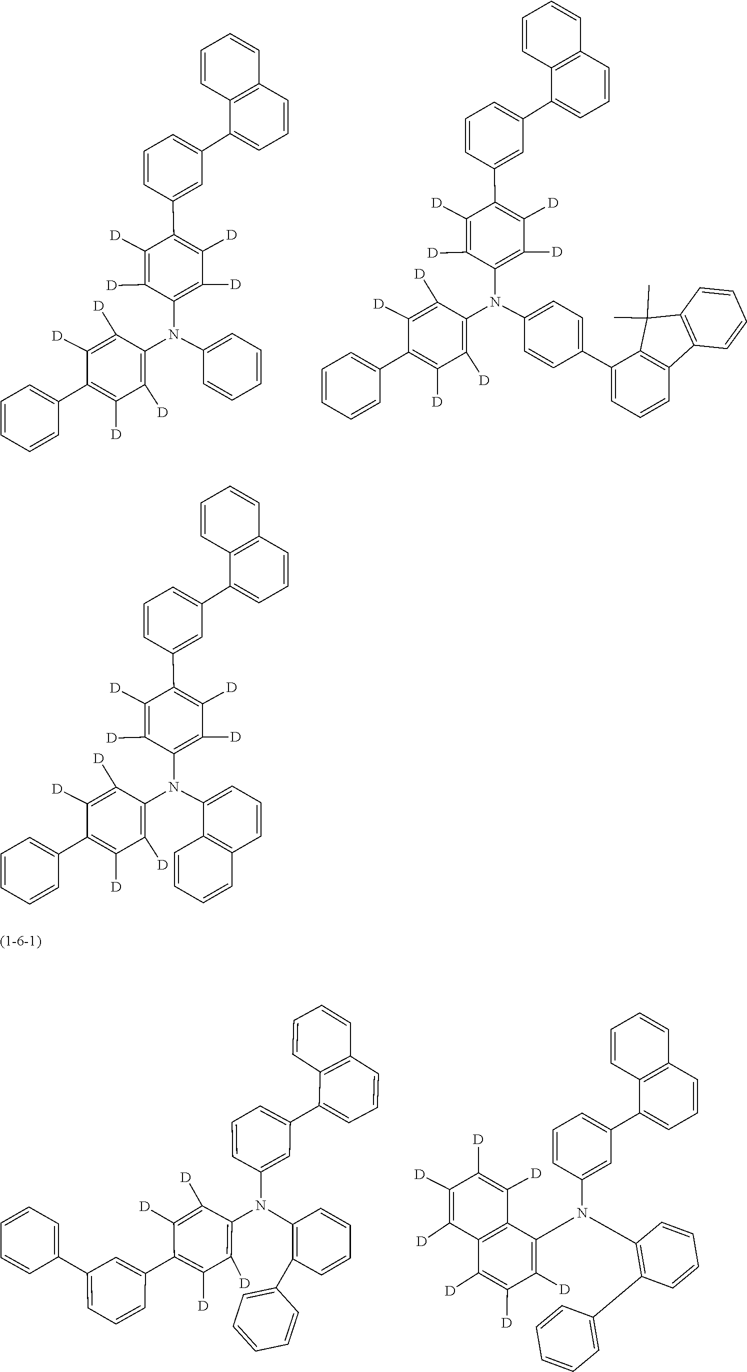

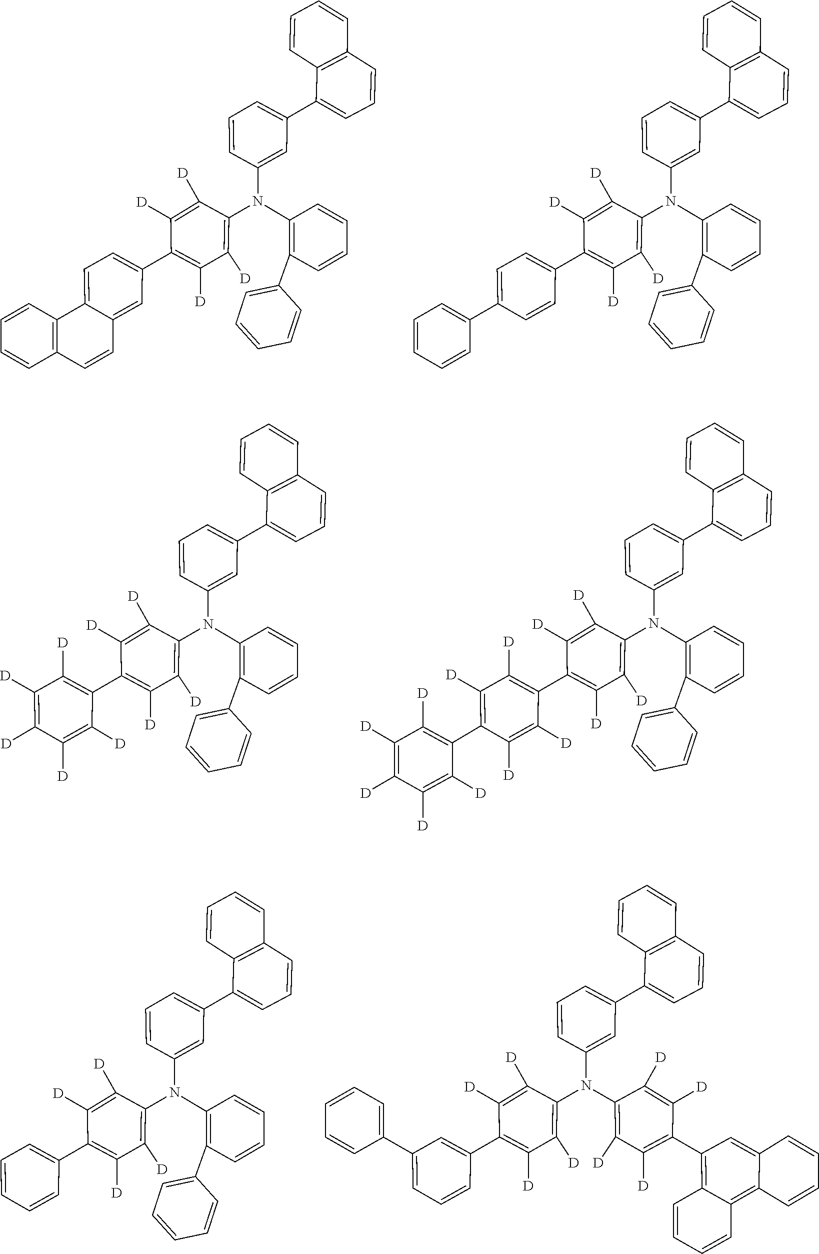

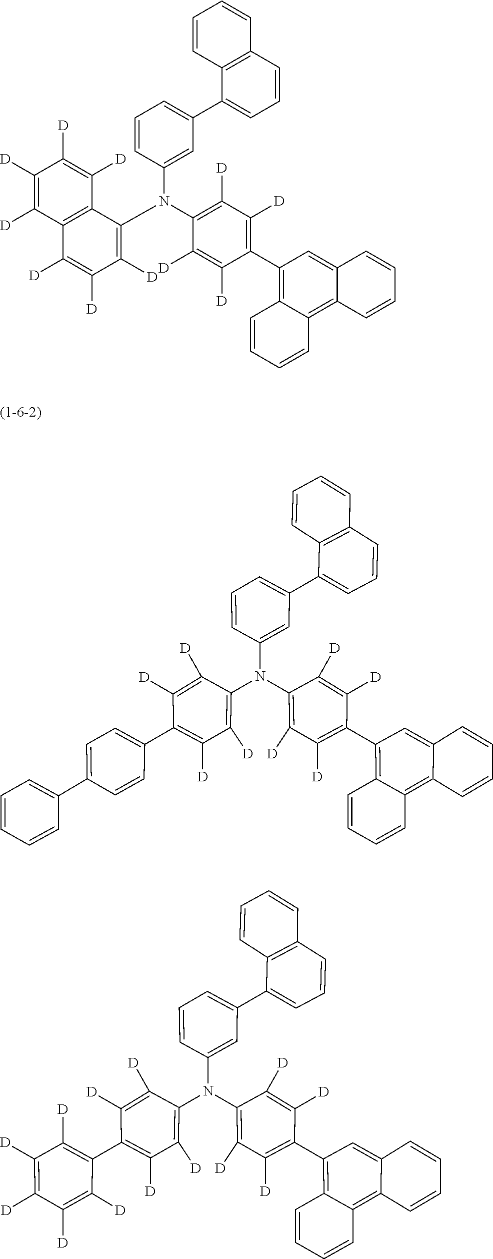 [SMILES: [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1cc2ccccc2c2ccccc12 ; [2H]c1c([2H])c(N(c2cccc(-c3cccc4ccccc34)c2)c2c([2H])c([2H])c(-c3cc4ccccc4c4ccccc34)c([2H])c2[2H])c([2H])c([2H])c1-c1ccc(-c2ccccc2)cc1 ; [2H]c1c([2H])c([2H])c(-c2c([2H])c([2H])c(N(c3cccc(-c4cccc5ccccc45)c3)c3c([2H])c([2H])c(-c4cc5ccccc5c5ccccc45)c([2H])c3[2H])c([2H])c2[2H])c([2H])c1[2H]]

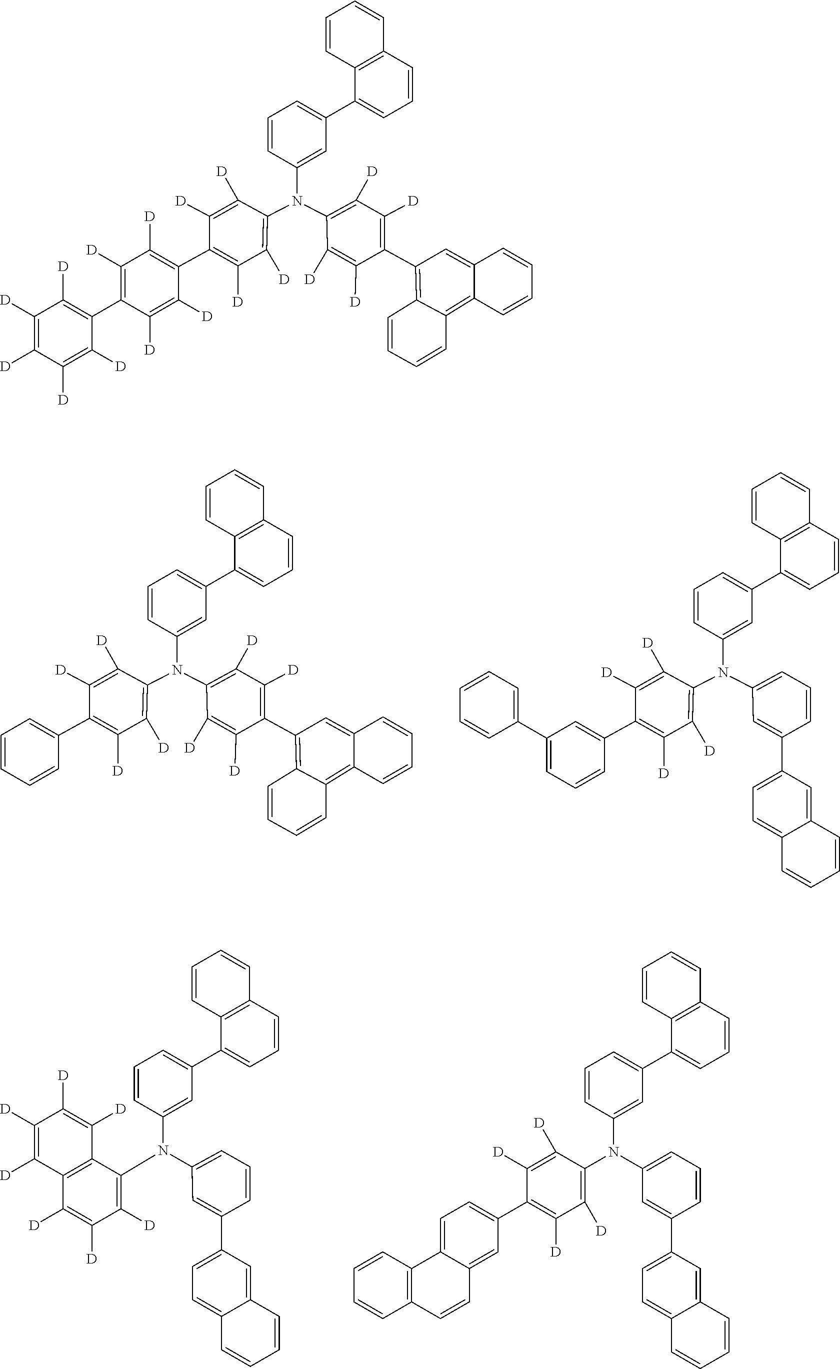

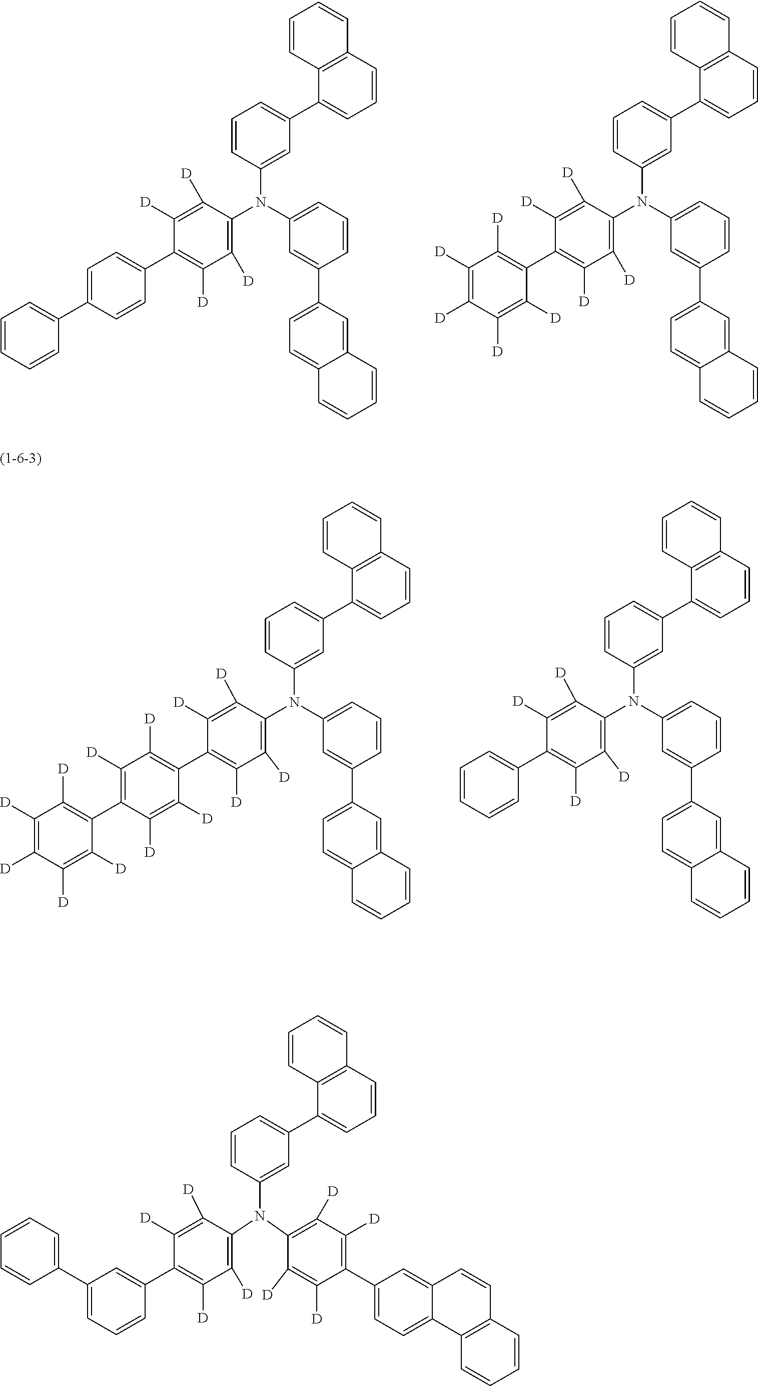

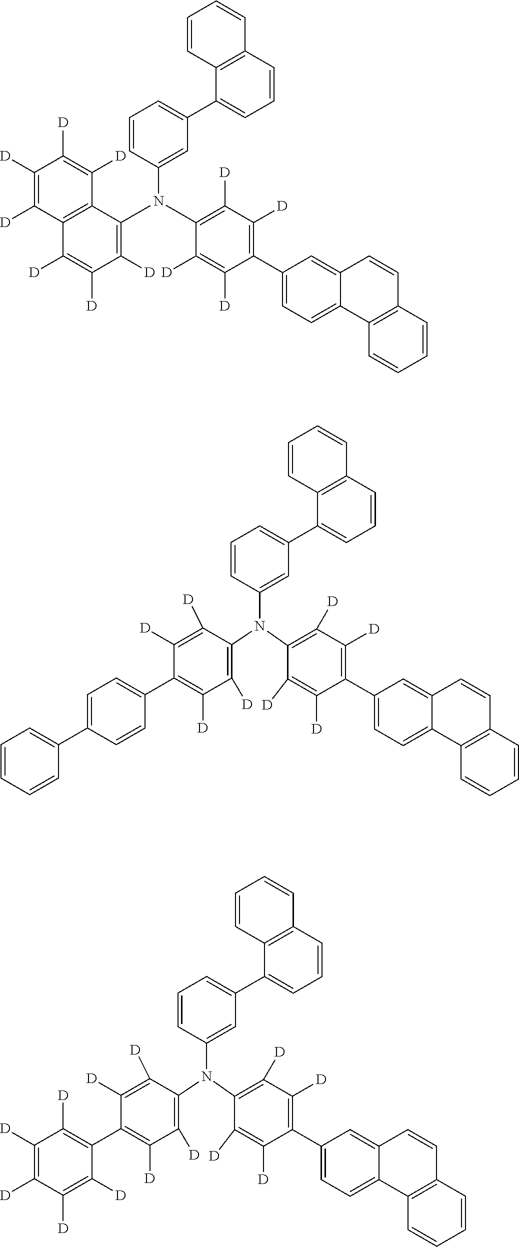 [SMILES: [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1ccc2c(ccc3ccccc32)c1 ; [2H]c1c([2H])c(N(c2cccc(-c3cccc4ccccc34)c2)c2c([2H])c([2H])c(-c3ccc4c(ccc5ccccc54)c3)c([2H])c2[2H])c([2H])c([2H])c1-c1ccc(-c2ccccc2)cc1 ; [2H]c1c([2H])c([2H])c(-c2c([2H])c([2H])c(N(c3cccc(-c4cccc5ccccc45)c3)c3c([2H])c([2H])c(-c4ccc5c(ccc6ccccc65)c4)c([2H])c3[2H])c([2H])c2[2H])c([2H])c1[2H]]

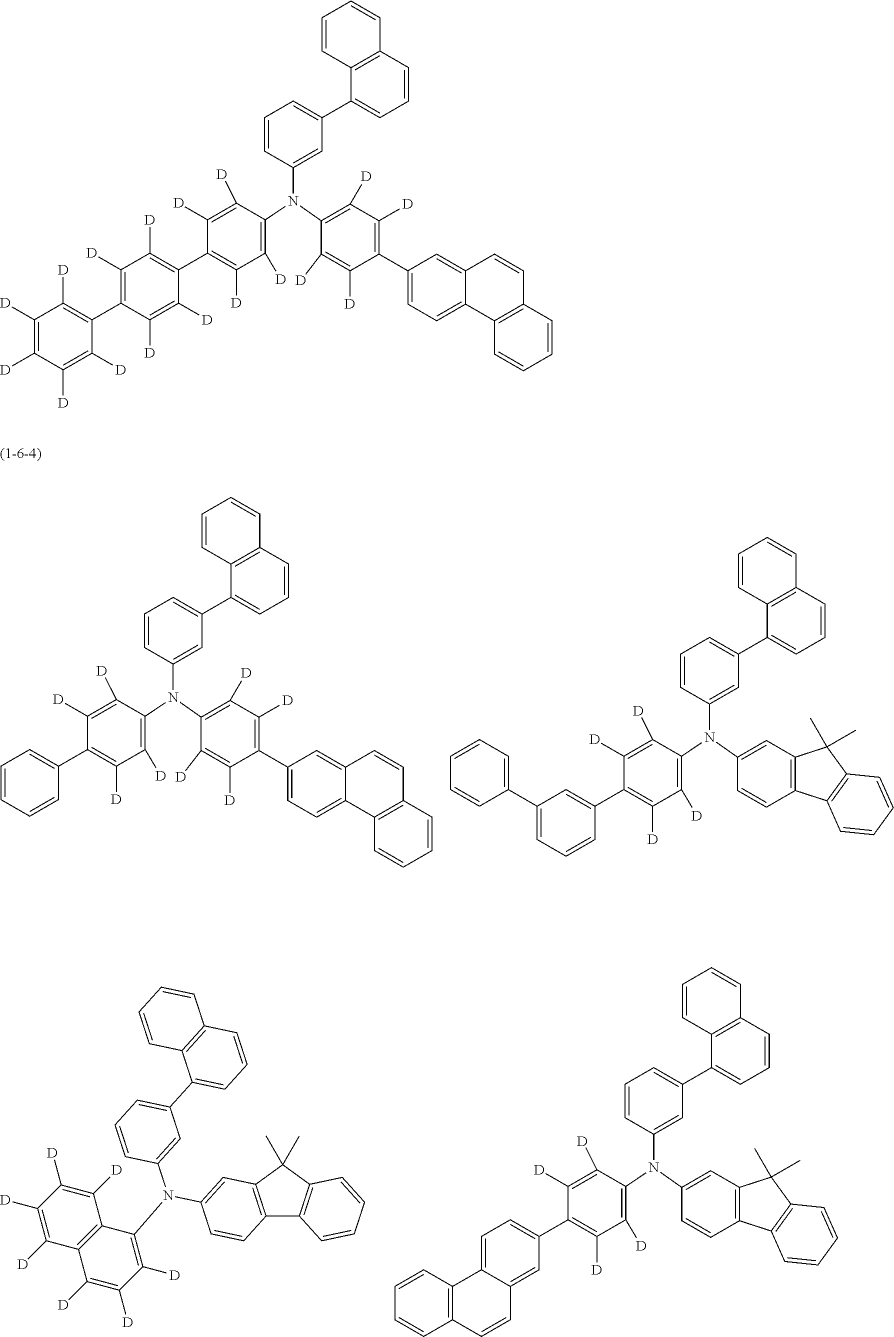

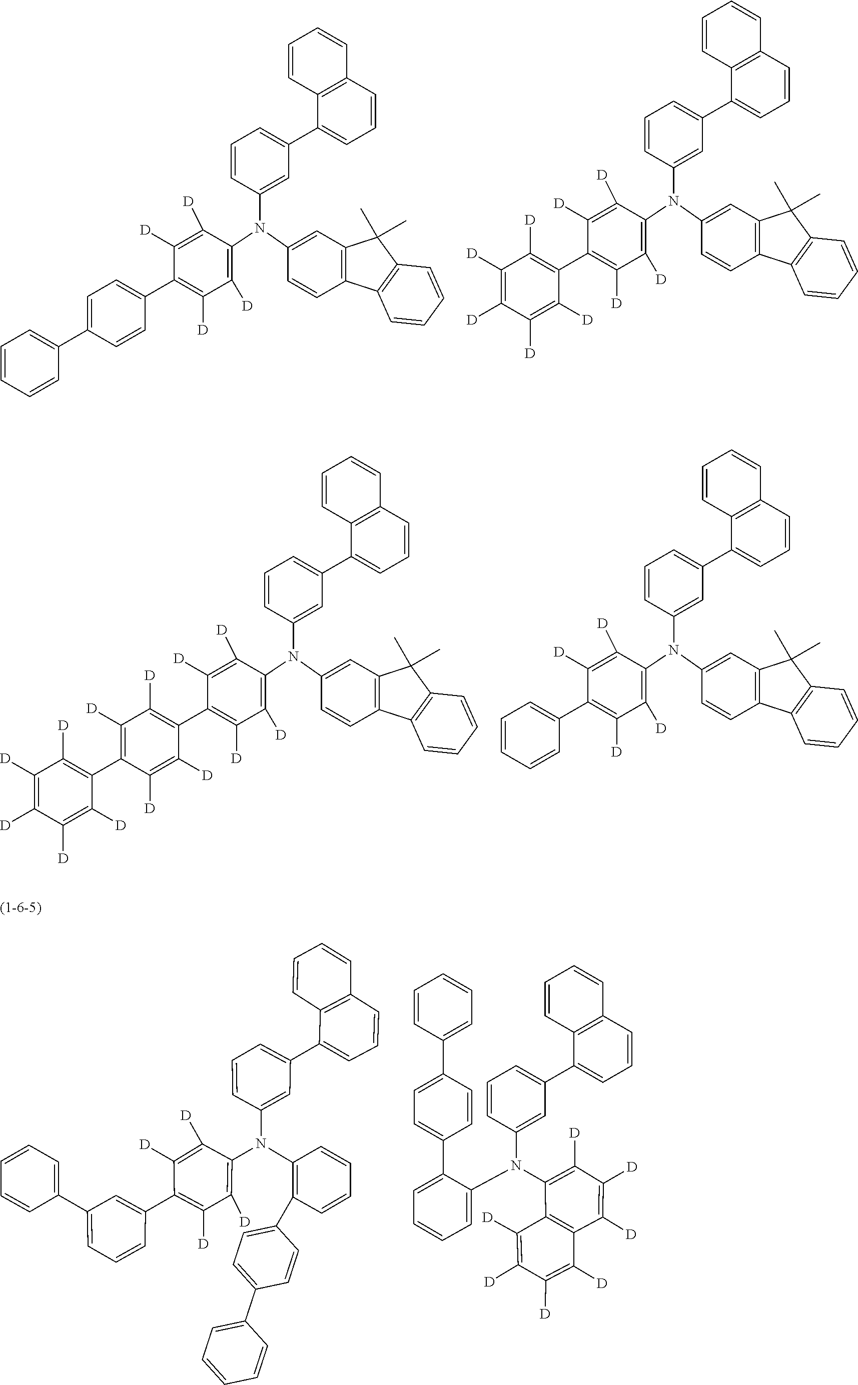

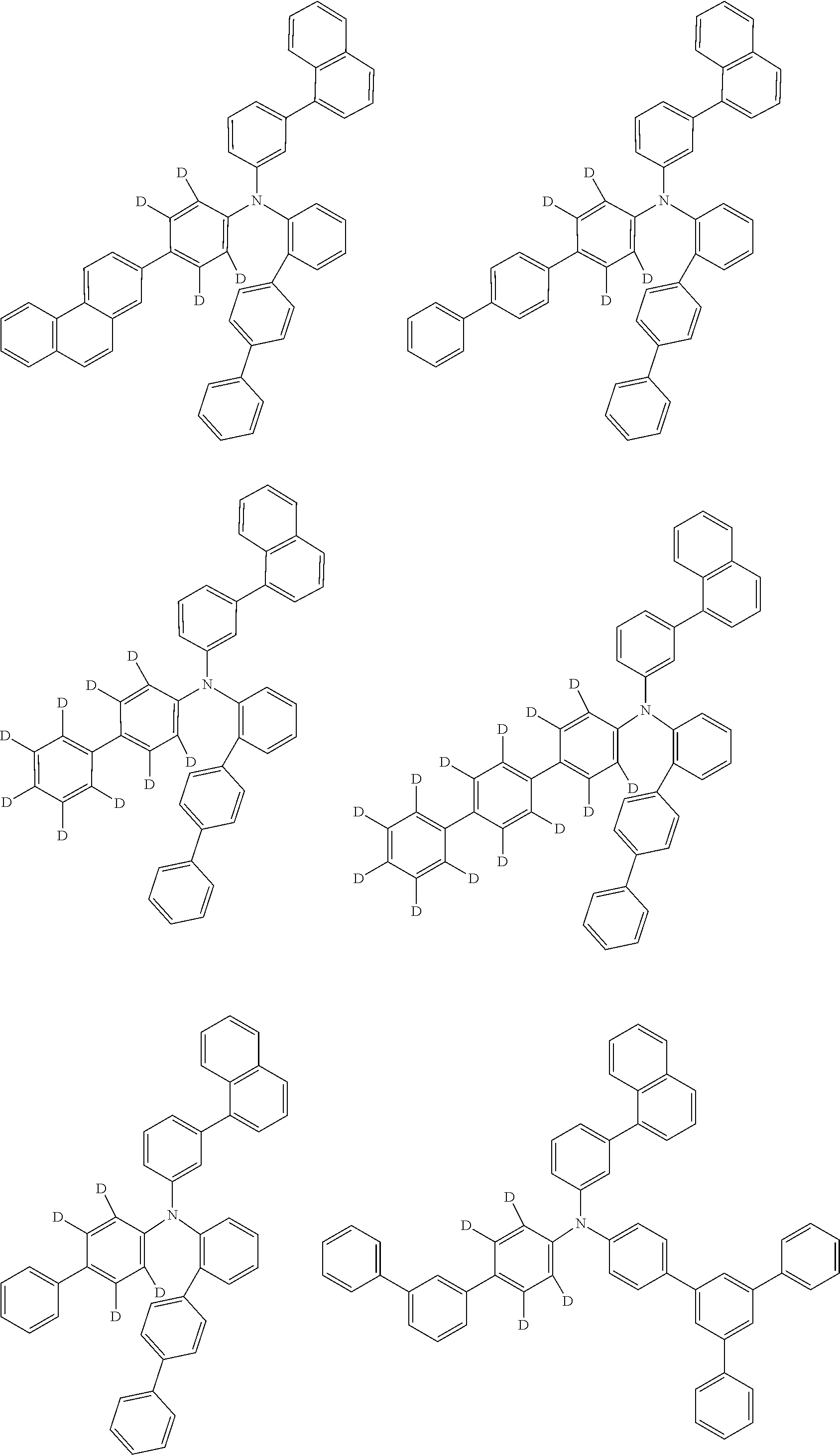

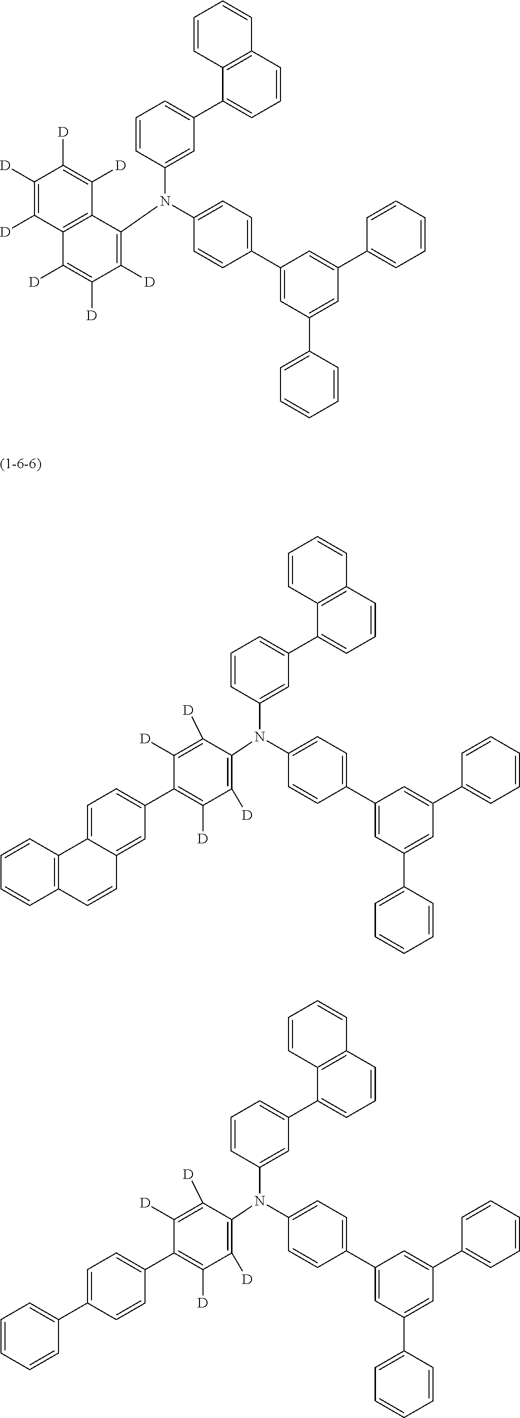 [SMILES: [2H]c1c([2H])c([2H])c2c(N(c3ccc(-c4cc(-c5ccccc5)cc(-c5ccccc5)c4)cc3)c3cccc(-c4cccc5ccccc45)c3)c([2H])c([2H])c([2H])c2c1[2H] ; [2H]c1c([2H])c(N(c2ccc(-c3cc(-c4ccccc4)cc(-c4ccccc4)c3)cc2)c2cccc(-c3cccc4ccccc34)c2)c([2H])c([2H])c1-c1ccc2c(ccc3ccccc32)c1 ; [2H]c1c([2H])c(N(c2ccc(-c3cc(-c4ccccc4)cc(-c4ccccc4)c3)cc2)c2cccc(-c3cccc4ccccc34)c2)c([2H])c([2H])c1-c1ccc(-c2ccccc2)cc1]

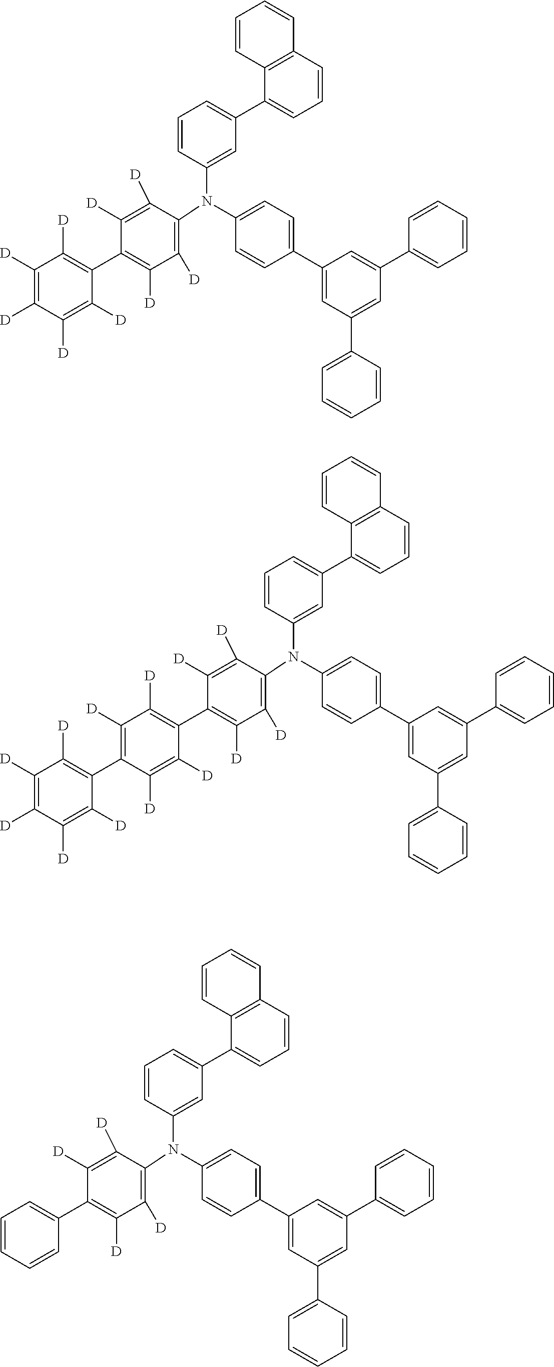

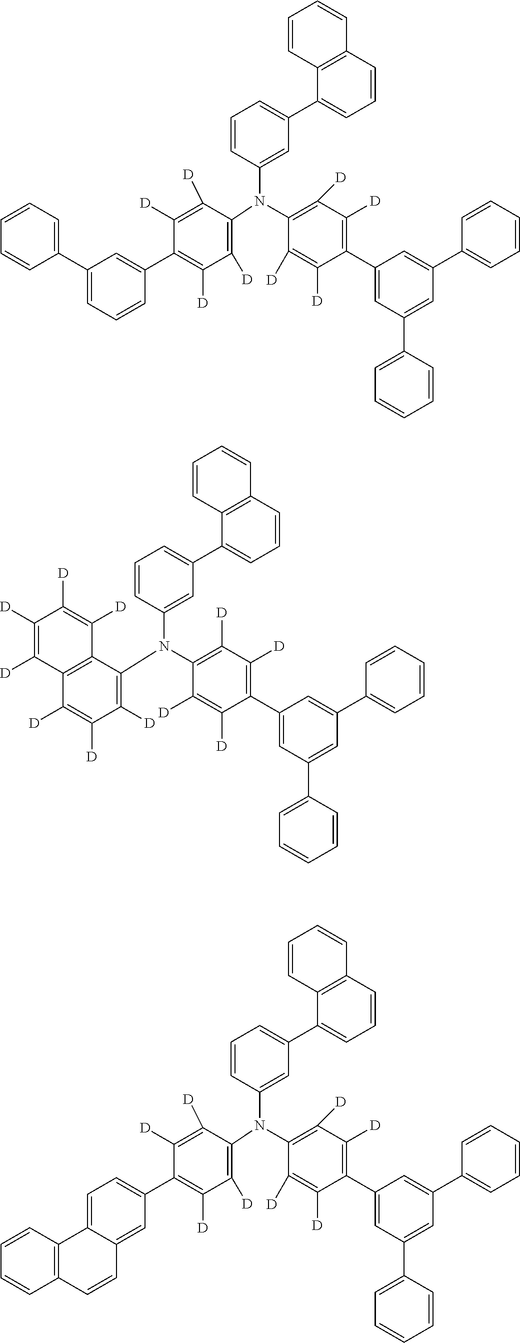 [SMILES: [2H]c1c([2H])c(N(c2cccc(-c3cccc4ccccc34)c2)c2c([2H])c([2H])c(-c3cc(-c4ccccc4)cc(-c4ccccc4)c3)c([2H])c2[2H])c([2H])c([2H])c1-c1cccc(-c2ccccc2)c1 ; [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1cc(-c2ccccc2)cc(-c2ccccc2)c1 ; [2H]c1c([2H])c(N(c2cccc(-c3cccc4ccccc34)c2)c2c([2H])c([2H])c(-c3ccc4c(ccc5ccccc54)c3)c([2H])c2[2H])c([2H])c([2H])c1-c1cc(-c2ccccc2)cc(-c2ccccc2)c1]

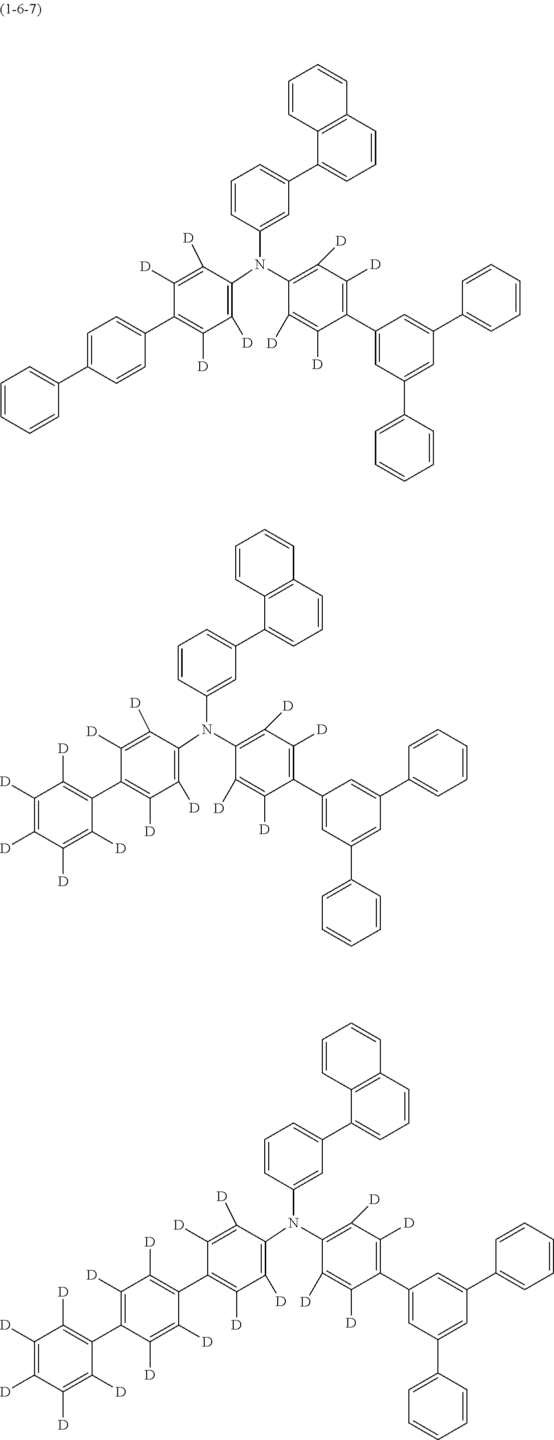

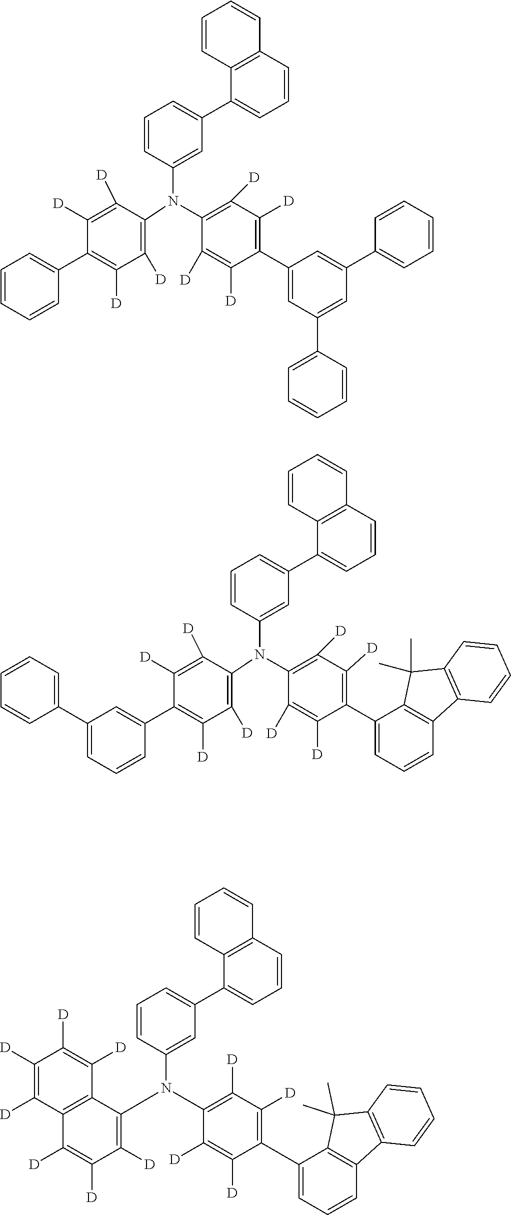

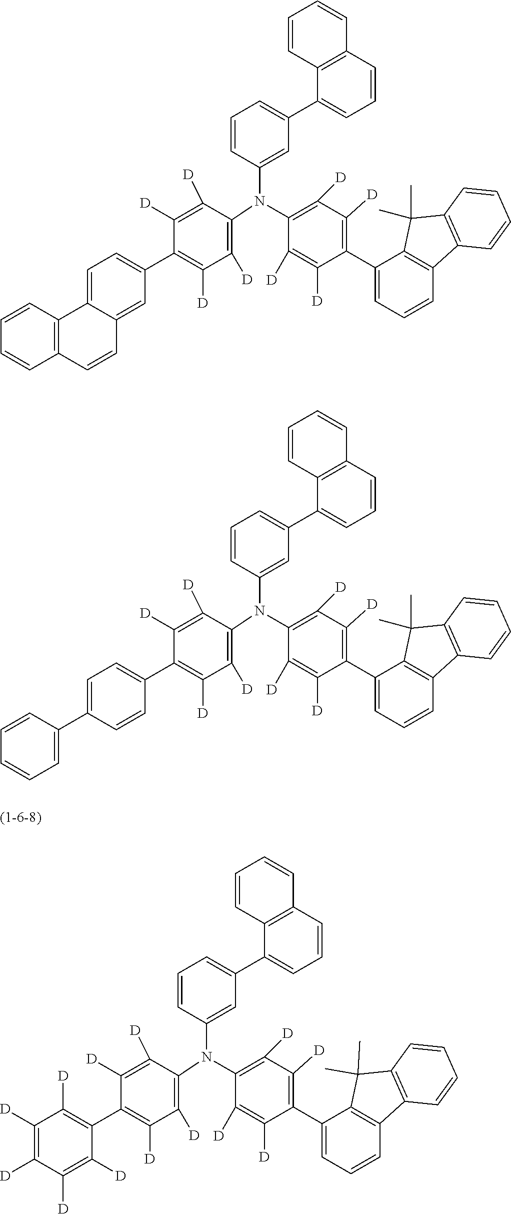 [SMILES: [2H]c1c([2H])c(N(c2cccc(-c3cccc4ccccc34)c2)c2c([2H])c([2H])c(-c3cccc4c3C(C)(C)c3ccccc3-4)c([2H])c2[2H])c([2H])c([2H])c1-c1ccc2c(ccc3ccccc32)c1 ; [2H]c1c([2H])c(N(c2cccc(-c3cccc4ccccc34)c2)c2c([2H])c([2H])c(-c3cccc4c3C(C)(C)c3ccccc3-4)c([2H])c2[2H])c([2H])c([2H])c1-c1ccc(-c2ccccc2)cc1 ; [2H]c1c([2H])c([2H])c(-c2c([2H])c([2H])c(N(c3cccc(-c4cccc5ccccc45)c3)c3c([2H])c([2H])c(-c4cccc5c4C(C)(C)c4ccccc4-5)c([2H])c3[2H])c([2H])c2[2H])c([2H])c1[2H]]

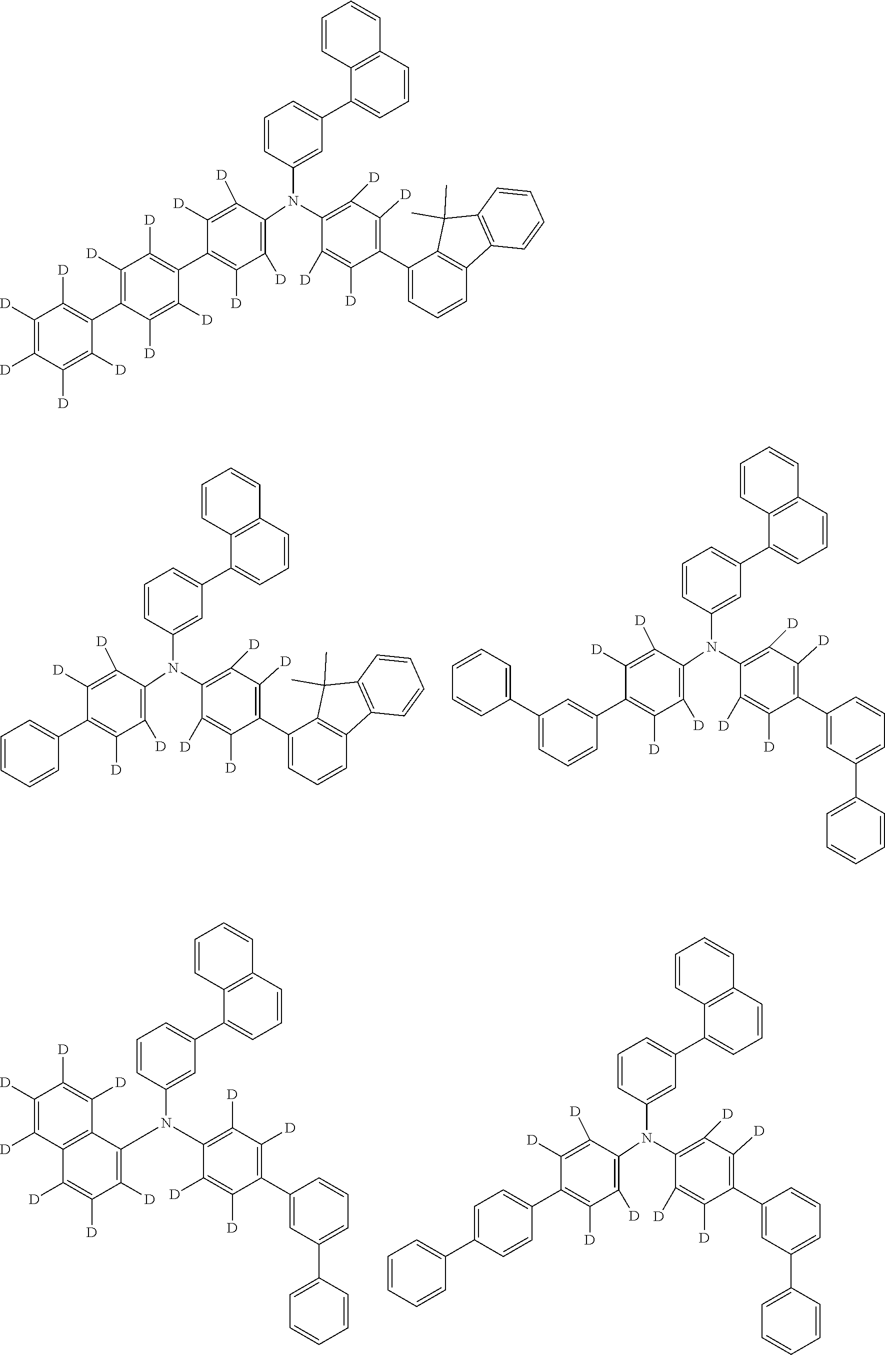

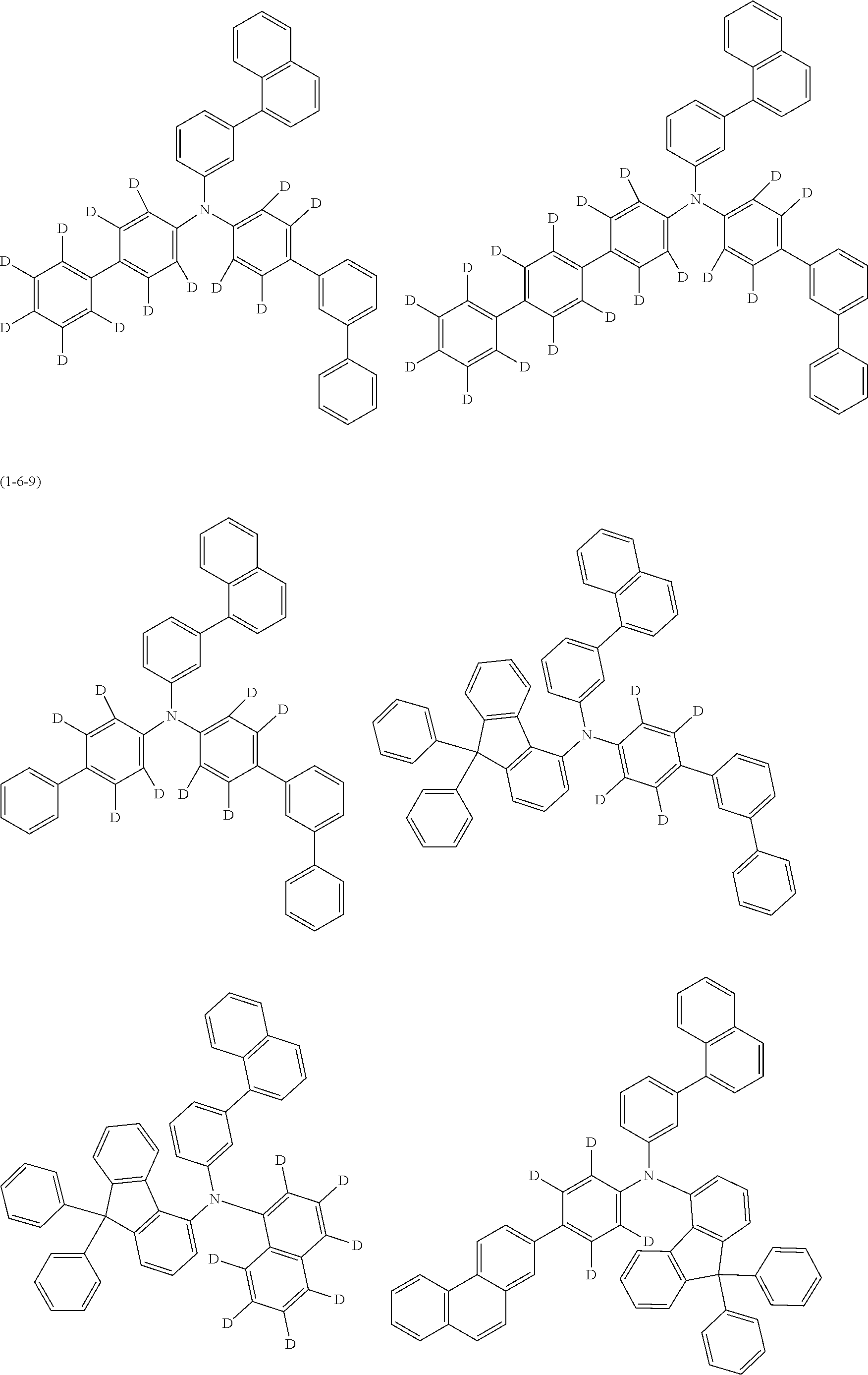

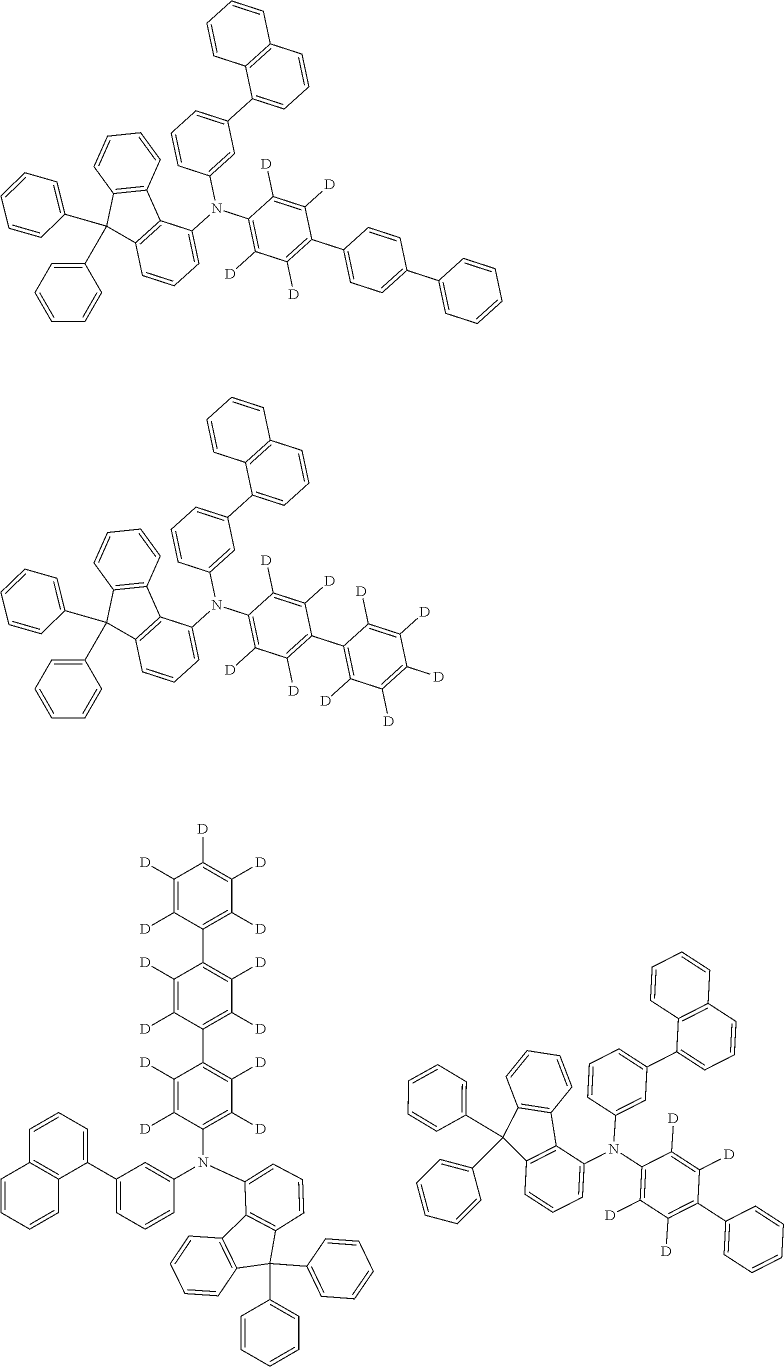

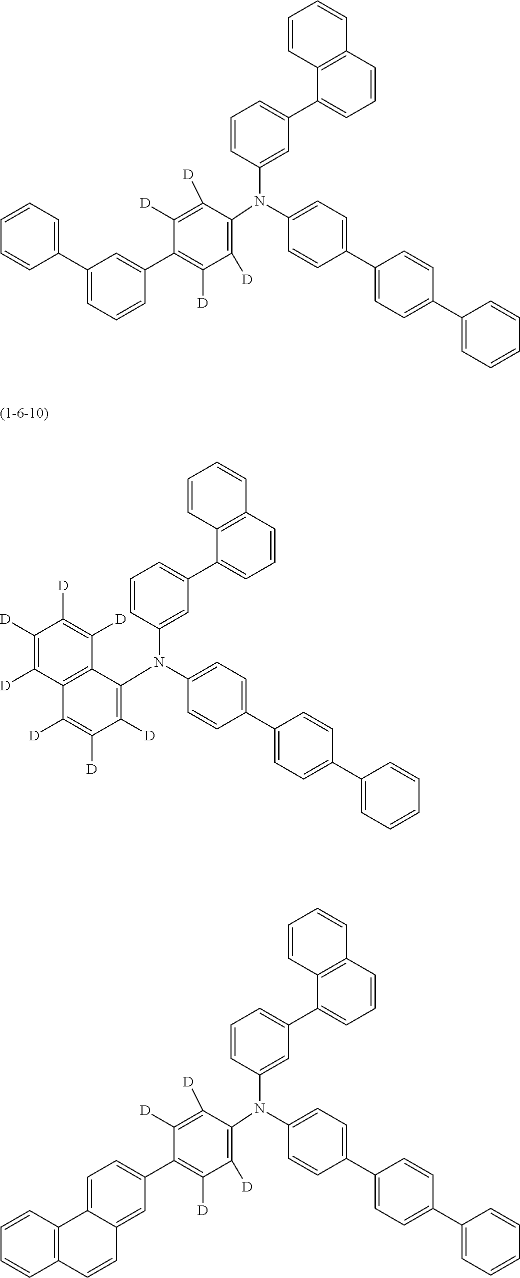 [SMILES: [2H]c1c([2H])c(N(c2ccc(-c3ccc(-c4ccccc4)cc3)cc2)c2cccc(-c3cccc4ccccc34)c2)c([2H])c([2H])c1-c1cccc(-c2ccccc2)c1 ; [2H]c1c([2H])c([2H])c2c(N(c3ccc(-c4ccc(-c5ccccc5)cc4)cc3)c3cccc(-c4cccc5ccccc45)c3)c([2H])c([2H])c([2H])c2c1[2H] ; [2H]c1c([2H])c(N(c2ccc(-c3ccc(-c4ccccc4)cc3)cc2)c2cccc(-c3cccc4ccccc34)c2)c([2H])c([2H])c1-c1ccc2c(ccc3ccccc32)c1]

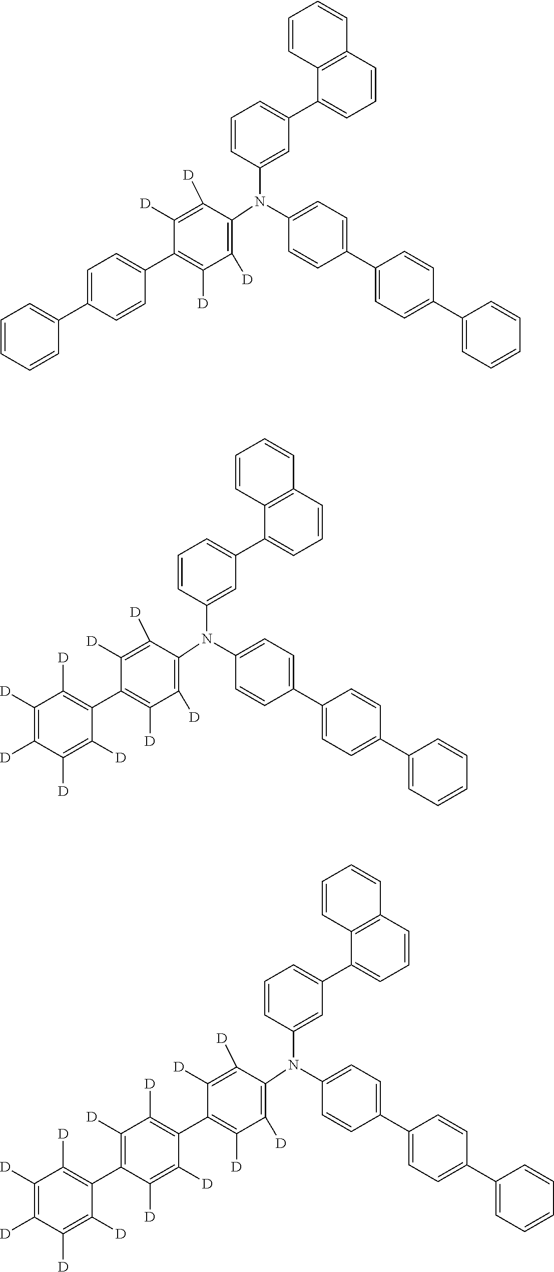 [SMILES: [2H]c1c([2H])c(N(c2ccc(-c3ccc(-c4ccccc4)cc3)cc2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3ccc(-c4ccc(-c5ccccc5)cc4)cc3)c3cccc(-c4cccc5ccccc45)c3)c([2H])c2[2H])c([2H])c1[2H] ; [2H]c1c([2H])c([2H])c(-c2c([2H])c([2H])c(-c3c([2H])c([2H])c(N(c4ccc(-c5ccc(-c6ccccc6)cc5)cc4)c4cccc(-c5cccc6ccccc56)c4)c([2H])c3[2H])c([2H])c2[2H])c([2H])c1[2H]]

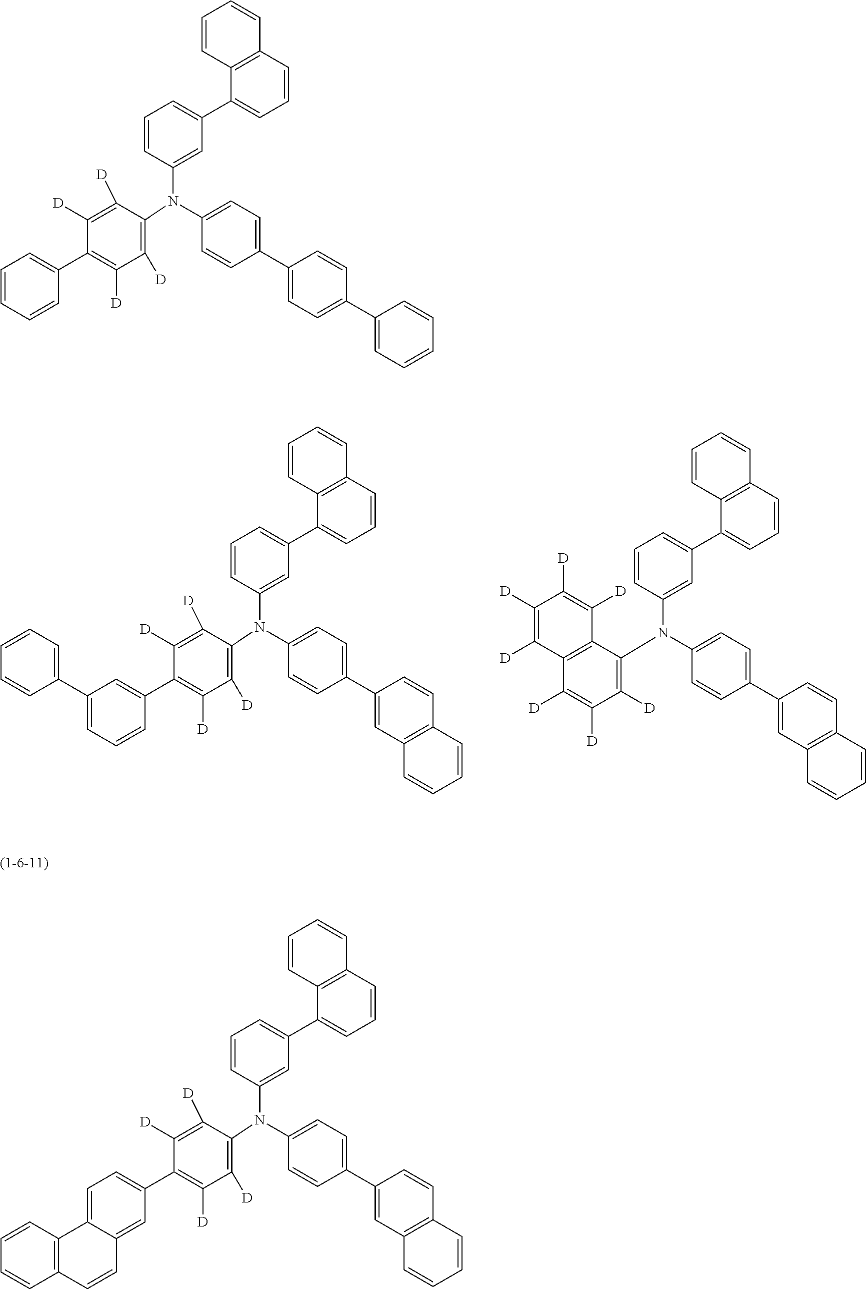 [SMILES: [2H]c1c([2H])c(N(c2ccc(-c3ccc(-c4ccccc4)cc3)cc2)c2cccc(-c3cccc4ccccc34)c2)c([2H])c([2H])c1-c1ccccc1 ; [2H]c1c([2H])c(N(c2ccc(-c3ccc4ccccc4c3)cc2)c2cccc(-c3cccc4ccccc34)c2)c([2H])c([2H])c1-c1cccc(-c2ccccc2)c1 ; [2H]c1c([2H])c([2H])c2c(N(c3ccc(-c4ccc5ccccc5c4)cc3)c3cccc(-c4cccc5ccccc45)c3)c([2H])c([2H])c([2H])c2c1[2H] ; [2H]c1c([2H])c(N(c2ccc(-c3ccc4ccccc4c3)cc2)c2cccc(-c3cccc4ccccc34)c2)c([2H])c([2H])c1-c1ccc2c(ccc3ccccc32)c1]

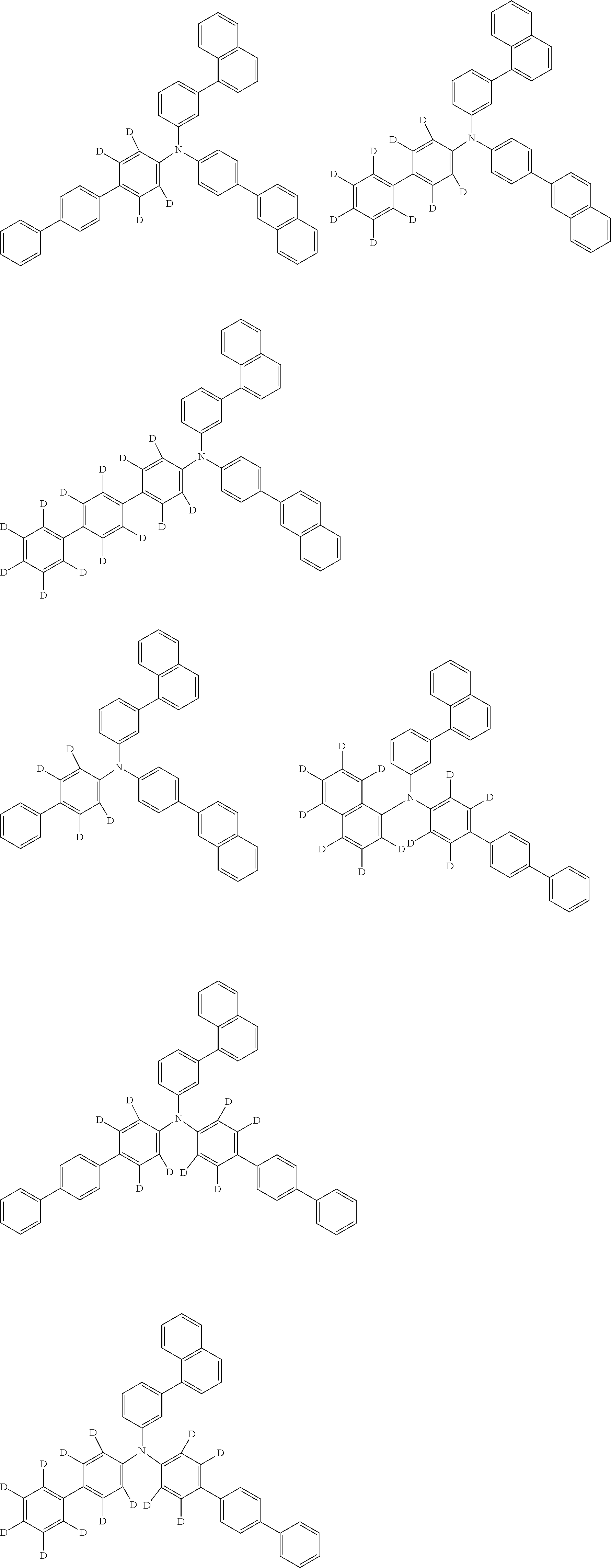

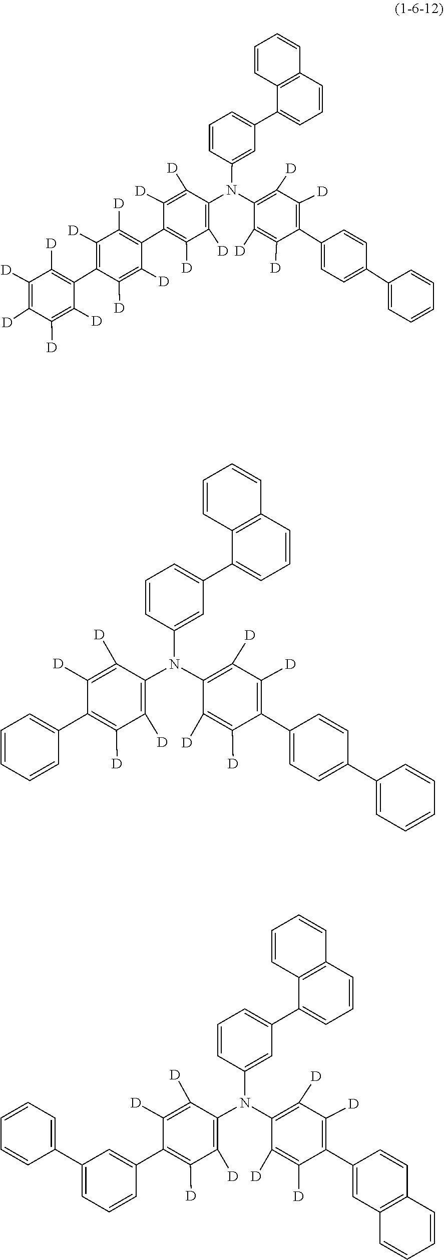 [SMILES: [2H]c1c([2H])c([2H])c(-c2c([2H])c([2H])c(-c3c([2H])c([2H])c(N(c4cccc(-c5cccc6ccccc56)c4)c4c([2H])c([2H])c(-c5ccc(-c6ccccc6)cc5)c([2H])c4[2H])c([2H])c3[2H])c([2H])c2[2H])c([2H])c1[2H] ; [2H]c1c([2H])c(N(c2cccc(-c3cccc4ccccc34)c2)c2c([2H])c([2H])c(-c3ccc(-c4ccccc4)cc3)c([2H])c2[2H])c([2H])c([2H])c1-c1ccccc1 ; [2H]c1c([2H])c(N(c2cccc(-c3cccc4ccccc34)c2)c2c([2H])c([2H])c(-c3ccc4ccccc4c3)c([2H])c2[2H])c([2H])c([2H])c1-c1cccc(-c2ccccc2)c1]

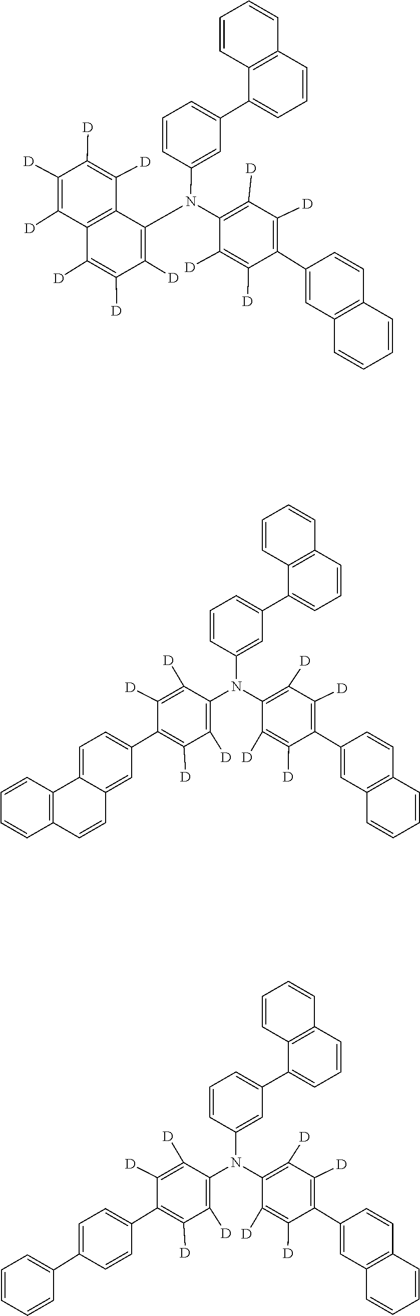 [SMILES: [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1ccc2ccccc2c1 ; [2H]c1c([2H])c(N(c2cccc(-c3cccc4ccccc34)c2)c2c([2H])c([2H])c(-c3ccc4c(ccc5ccccc54)c3)c([2H])c2[2H])c([2H])c([2H])c1-c1ccc2ccccc2c1 ; [2H]c1c([2H])c(N(c2cccc(-c3cccc4ccccc34)c2)c2c([2H])c([2H])c(-c3ccc4ccccc4c3)c([2H])c2[2H])c([2H])c([2H])c1-c1ccc(-c2ccccc2)cc1]

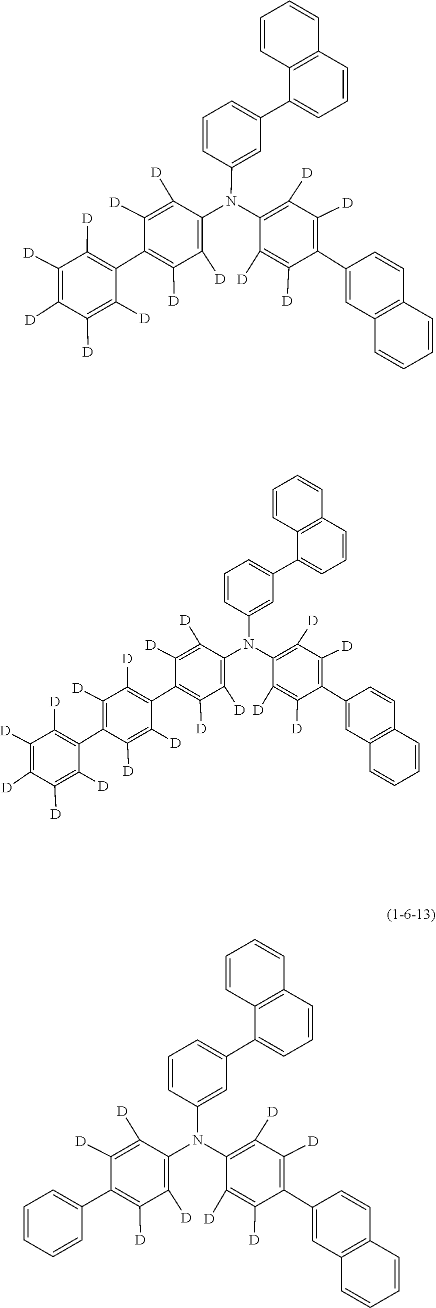 [SMILES: [2H]c1c([2H])c([2H])c(-c2c([2H])c([2H])c(N(c3cccc(-c4cccc5ccccc45)c3)c3c([2H])c([2H])c(-c4ccc5ccccc5c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5cccc6ccccc56)c4)c4c([2H])c([2H])c(-c5ccc6ccccc6c5)c([2H])c4[2H])c([2H])c3[2H])c([2H])c2[2H])c([2H])c1[2H] ; [2H]c1c([2H])c(N(c2cccc(-c3cccc4ccccc34)c2)c2c([2H])c([2H])c(-c3ccc4ccccc4c3)c([2H])c2[2H])c([2H])c([2H])c1-c1ccccc1]

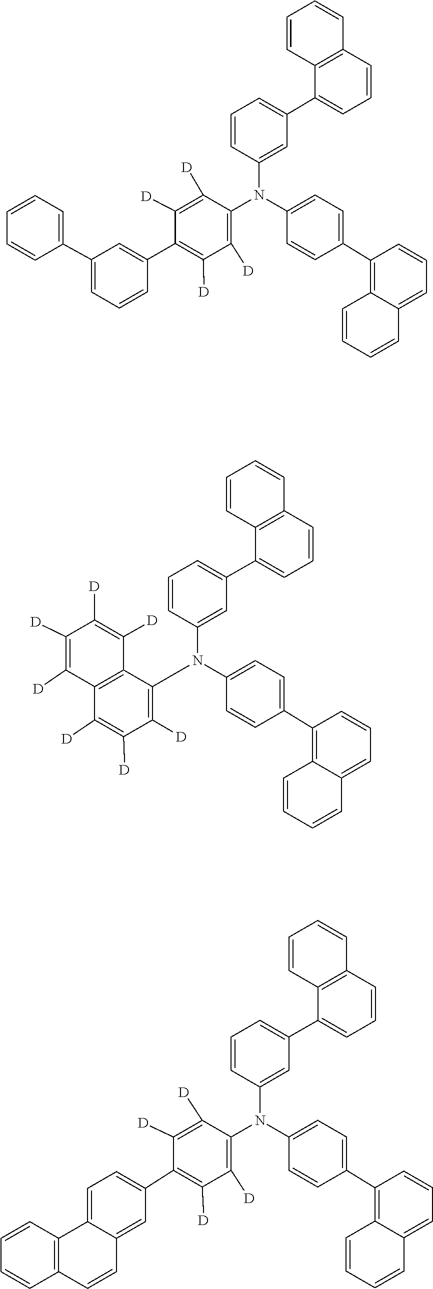 [SMILES: [2H]c1c([2H])c(N(c2ccc(-c3cccc4ccccc34)cc2)c2cccc(-c3cccc4ccccc34)c2)c([2H])c([2H])c1-c1cccc(-c2ccccc2)c1 ; [2H]c1c([2H])c([2H])c2c(N(c3ccc(-c4cccc5ccccc45)cc3)c3cccc(-c4cccc5ccccc45)c3)c([2H])c([2H])c([2H])c2c1[2H] ; [2H]c1c([2H])c(N(c2ccc(-c3cccc4ccccc34)cc2)c2cccc(-c3cccc4ccccc34)c2)c([2H])c([2H])c1-c1ccc2c(ccc3ccccc32)c1]

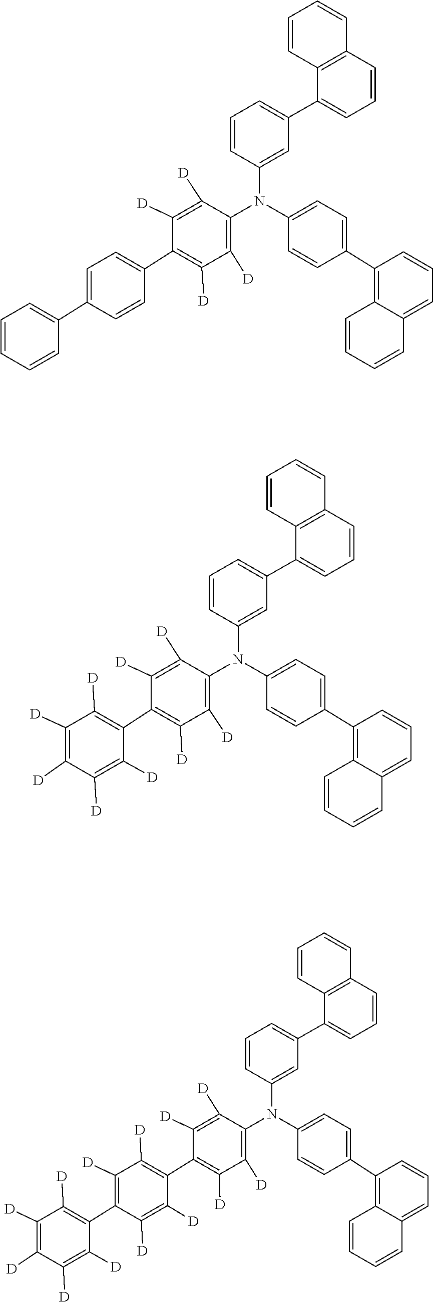 [SMILES: [2H]c1c([2H])c(N(c2ccc(-c3cccc4ccccc34)cc2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3ccc(-c4cccc5ccccc45)cc3)c3cccc(-c4cccc5ccccc45)c3)c([2H])c2[2H])c([2H])c1[2H] ; [2H]c1c([2H])c([2H])c(-c2c([2H])c([2H])c(-c3c([2H])c([2H])c(N(c4ccc(-c5cccc6ccccc56)cc4)c4cccc(-c5cccc6ccccc56)c4)c([2H])c3[2H])c([2H])c2[2H])c([2H])c1[2H]]

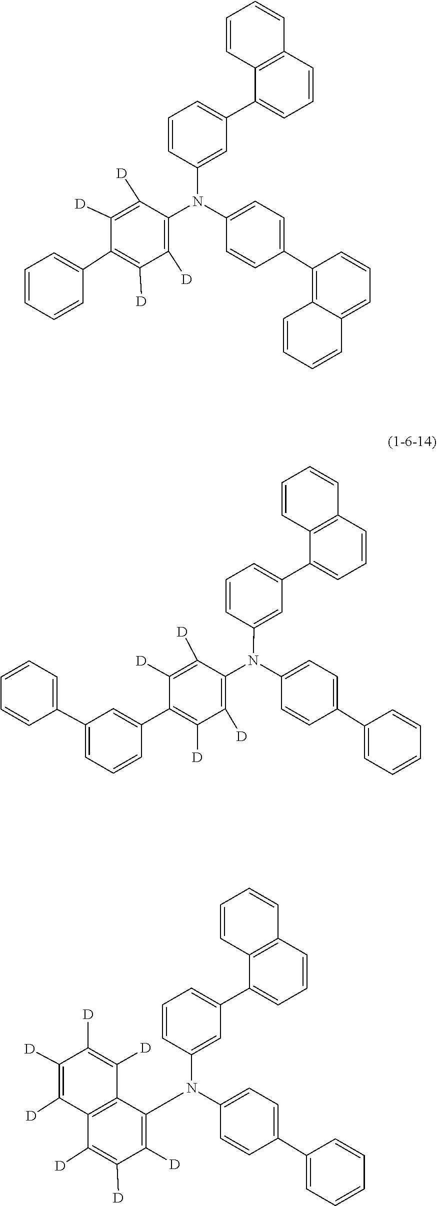 [SMILES: [2H]c1c([2H])c(N(c2ccc(-c3cccc4ccccc34)cc2)c2cccc(-c3cccc4ccccc34)c2)c([2H])c([2H])c1-c1ccccc1 ; [2H]c1c([2H])c(N(c2ccc(-c3ccccc3)cc2)c2cccc(-c3cccc4ccccc34)c2)c([2H])c([2H])c1-c1cccc(-c2ccccc2)c1 ; [2H]c1c([2H])c([2H])c2c(N(c3ccc(-c4ccccc4)cc3)c3cccc(-c4cccc5ccccc45)c3)c([2H])c([2H])c([2H])c2c1[2H]]

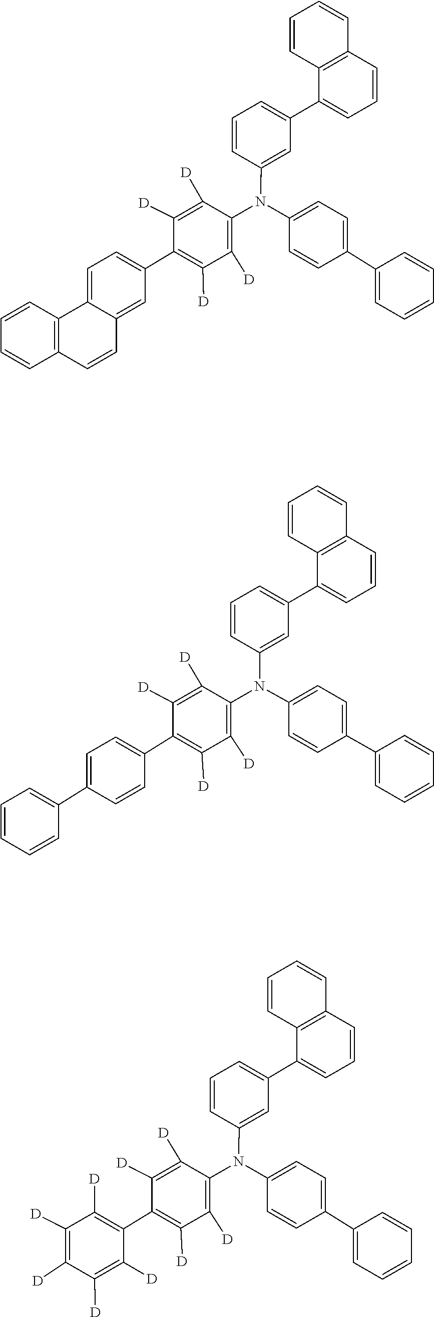 [SMILES: [2H]c1c([2H])c(N(c2ccc(-c3ccccc3)cc2)c2cccc(-c3cccc4ccccc34)c2)c([2H])c([2H])c1-c1ccc2c(ccc3ccccc32)c1 ; [2H]c1c([2H])c(N(c2ccc(-c3ccccc3)cc2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3ccc(-c4ccccc4)cc3)c3cccc(-c4cccc5ccccc45)c3)c([2H])c2[2H])c([2H])c1[2H]]

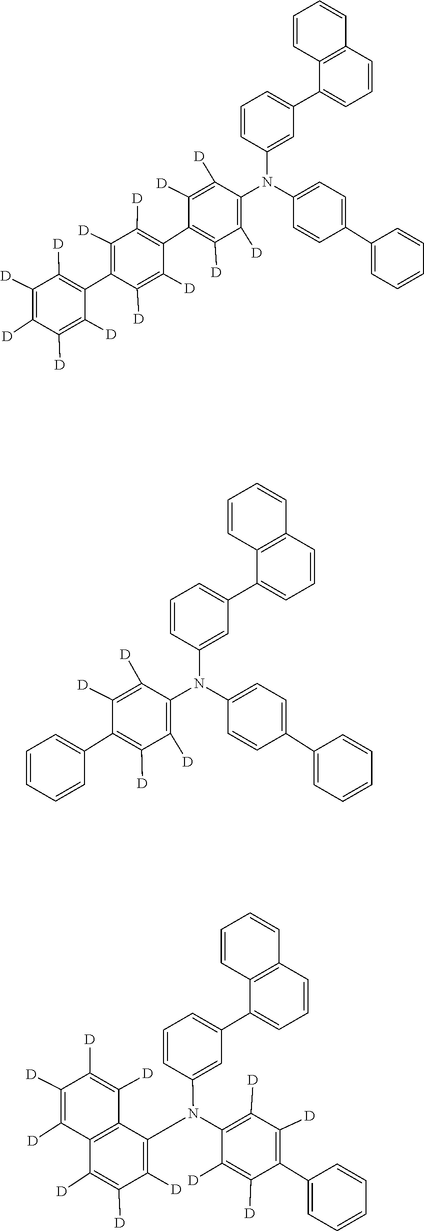 [SMILES: [2H]c1c([2H])c([2H])c(-c2c([2H])c([2H])c(-c3c([2H])c([2H])c(N(c4ccc(-c5ccccc5)cc4)c4cccc(-c5cccc6ccccc56)c4)c([2H])c3[2H])c([2H])c2[2H])c([2H])c1[2H] ; [2H]c1c([2H])c(N(c2ccc(-c3ccccc3)cc2)c2cccc(-c3cccc4ccccc34)c2)c([2H])c([2H])c1-c1ccccc1 ; [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1ccccc1]

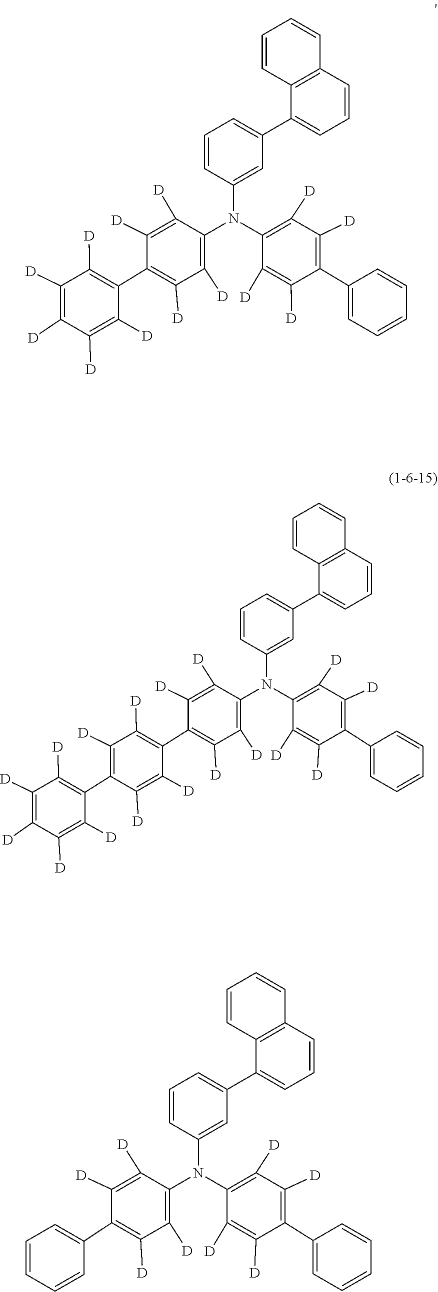 [SMILES: [2H]c1c([2H])c([2H])c(-c2c([2H])c([2H])c(N(c3cccc(-c4cccc5ccccc45)c3)c3c([2H])c([2H])c(-c4ccccc4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5cccc6ccccc56)c4)c4c([2H])c([2H])c(-c5ccccc5)c([2H])c4[2H])c([2H])c3[2H])c([2H])c2[2H])c([2H])c1[2H] ; [2H]c1c([2H])c(N(c2cccc(-c3cccc4ccccc34)c2)c2c([2H])c([2H])c(-c3ccccc3)c([2H])c2[2H])c([2H])c([2H])c1-c1ccccc1]

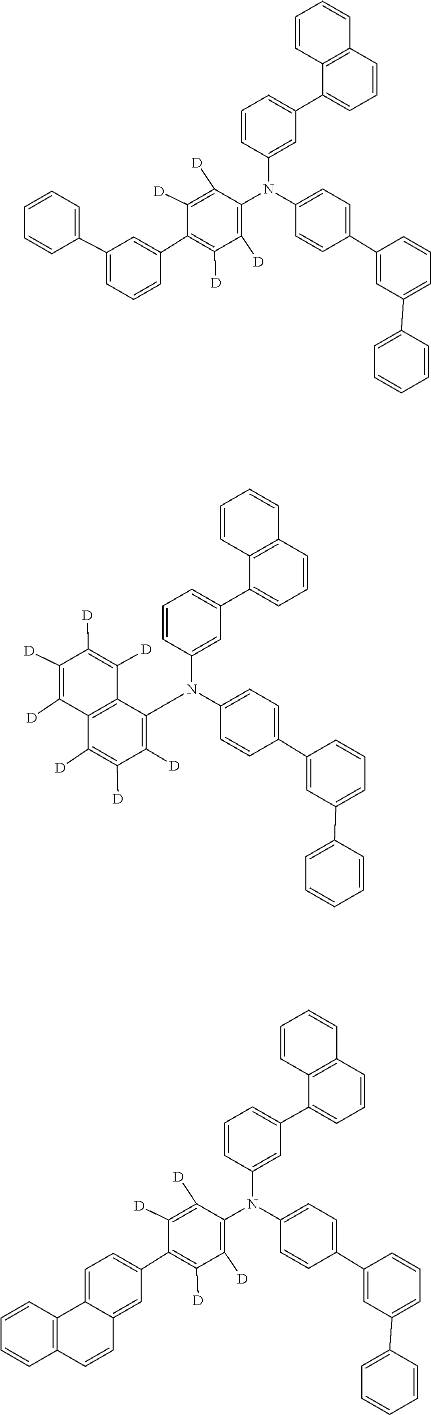 [SMILES: [2H]c1c([2H])c(N(c2ccc(-c3cccc(-c4ccccc4)c3)cc2)c2cccc(-c3cccc4ccccc34)c2)c([2H])c([2H])c1-c1cccc(-c2ccccc2)c1 ; [2H]c1c([2H])c([2H])c2c(N(c3ccc(-c4cccc(-c5ccccc5)c4)cc3)c3cccc(-c4cccc5ccccc45)c3)c([2H])c([2H])c([2H])c2c1[2H] ; [2H]c1c([2H])c(N(c2ccc(-c3cccc(-c4ccccc4)c3)cc2)c2cccc(-c3cccc4ccccc34)c2)c([2H])c([2H])c1-c1ccc2c(ccc3ccccc32)c1]

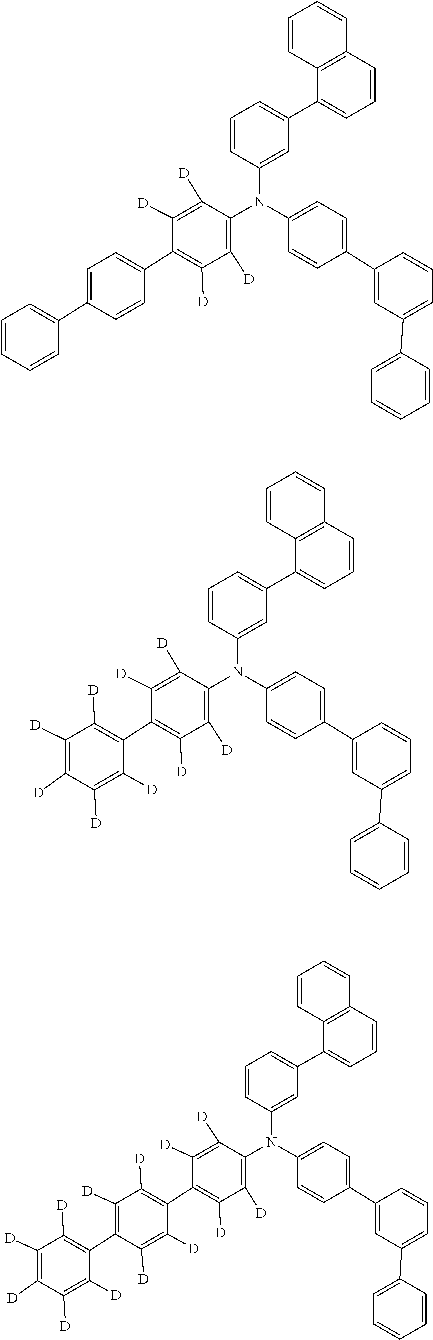 [SMILES: [2H]c1c([2H])c(N(c2ccc(-c3cccc(-c4ccccc4)c3)cc2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3ccc(-c4cccc(-c5ccccc5)c4)cc3)c3cccc(-c4cccc5ccccc45)c3)c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccccc6)c5)cc4)c4cccc(-c5cccc6ccccc56)c4)c([2H])c3[2H])c([2H])c2[2H])c([2H])c1[2H]]

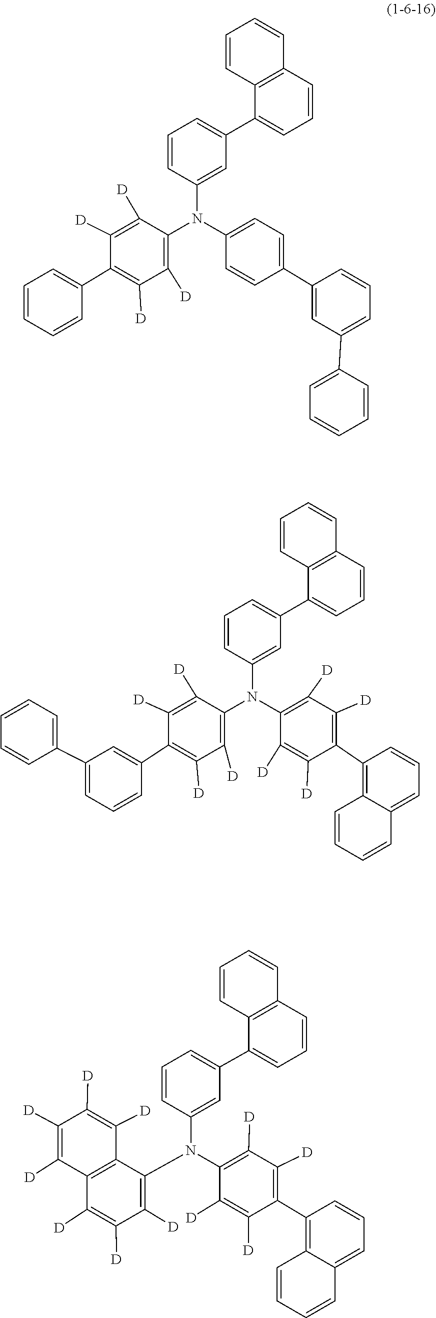 [SMILES: [2H]c1c([2H])c(N(c2ccc(-c3cccc(-c4ccccc4)c3)cc2)c2cccc(-c3cccc4ccccc34)c2)c([2H])c([2H])c1-c1ccccc1 ; [2H]c1c([2H])c(N(c2cccc(-c3cccc4ccccc34)c2)c2c([2H])c([2H])c(-c3cccc4ccccc34)c([2H])c2[2H])c([2H])c([2H])c1-c1cccc(-c2ccccc2)c1 ; [2H]c1c([2H])c(N(c2cccc(-c3cccc4ccccc34)c2)c2c([2H])c([2H])c([2H])c3c([2H])c([2H])c([2H])c([2H])c23)c([2H])c([2H])c1-c1cccc2ccccc12]

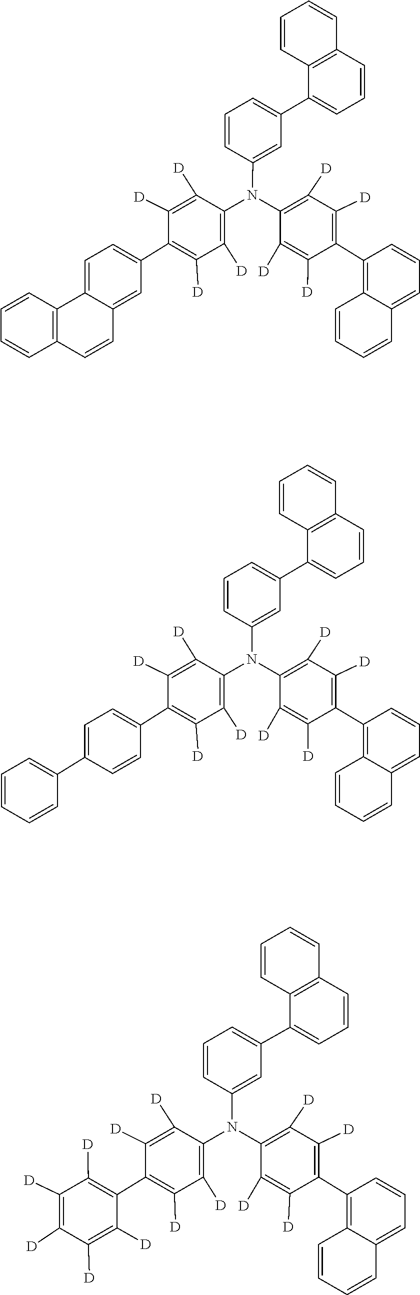 [SMILES: [2H]c1c([2H])c(N(c2cccc(-c3cccc4ccccc34)c2)c2c([2H])c([2H])c(-c3cccc4ccccc34)c([2H])c2[2H])c([2H])c([2H])c1-c1ccc2c(ccc3ccccc32)c1 ; [2H]c1c([2H])c(N(c2cccc(-c3cccc4ccccc34)c2)c2c([2H])c([2H])c(-c3cccc4ccccc34)c([2H])c2[2H])c([2H])c([2H])c1-c1ccc(-c2ccccc2)cc1 ; [2H]c1c([2H])c([2H])c(-c2c([2H])c([2H])c(N(c3cccc(-c4cccc5ccccc45)c3)c3c([2H])c([2H])c(-c4cccc5ccccc45)c([2H])c3[2H])c([2H])c2[2H])c([2H])c1[2H]]

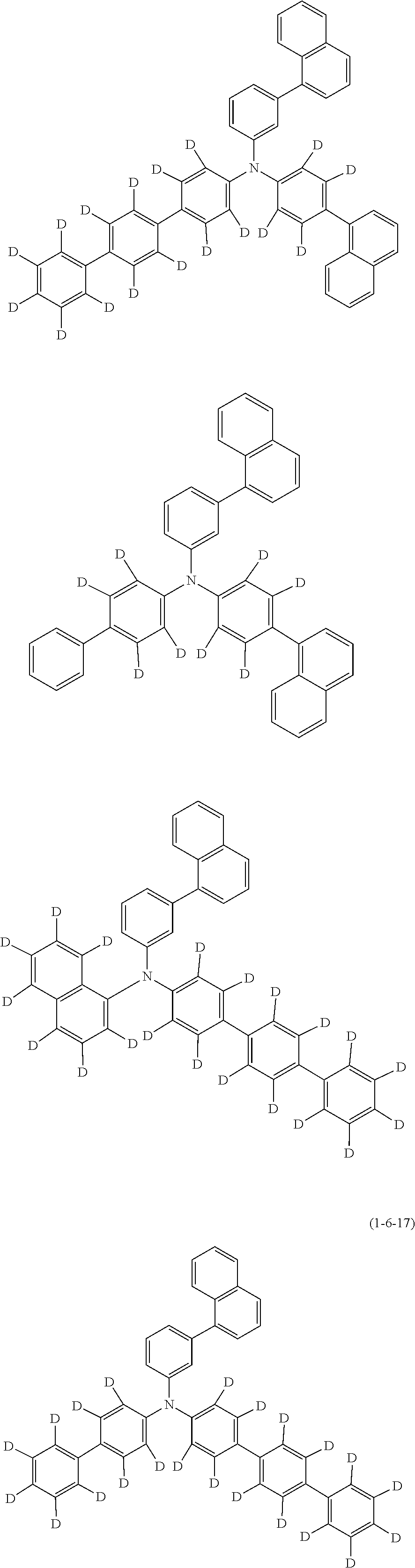

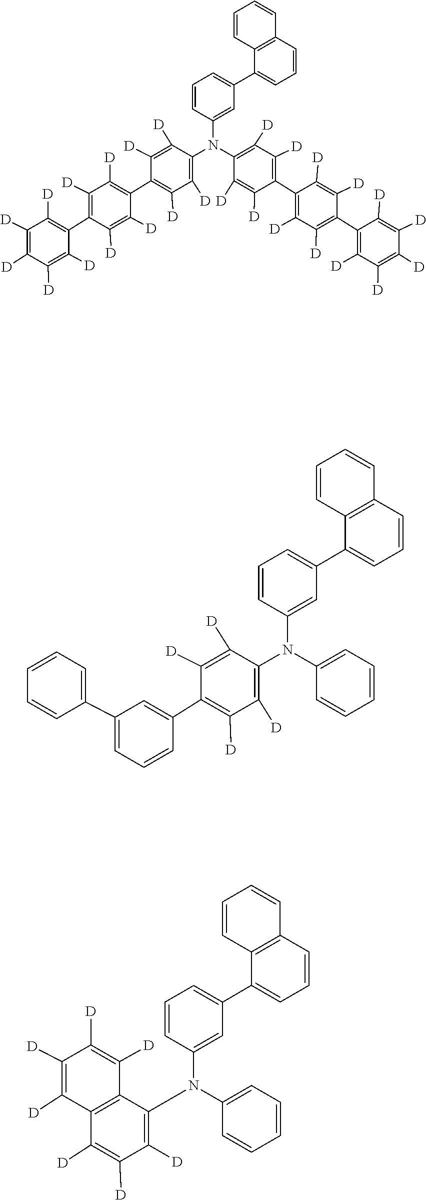 [SMILES: [2H]c1c([2H])c([2H])c(-c2c([2H])c([2H])c(-c3c([2H])c([2H])c(N(c4cccc(-c5cccc6ccccc56)c4)c4c([2H])c([2H])c(-c5c([2H])c([2H])c(-c6c([2H])c([2H])c([2H])c([2H])c6[2H])c([2H])c5[2H])c([2H])c4[2H])c([2H])c3[2H])c([2H])c2[2H])c([2H])c1[2H] ; [2H]c1c([2H])c(N(c2ccccc2)c2cccc(-c3cccc4ccccc34)c2)c([2H])c([2H])c1-c1cccc(-c2ccccc2)c1 ; [2H]c1c([2H])c([2H])c2c(N(c3ccccc3)c3cccc(-c4cccc5ccccc45)c3)c([2H])c([2H])c([2H])c2c1[2H]]

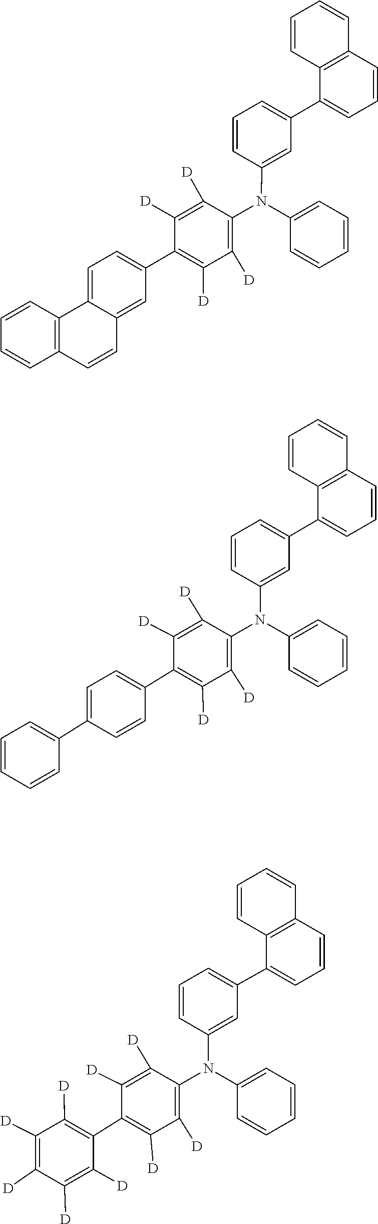 [SMILES: [2H]c1c([2H])c(N(c2ccccc2)c2cccc(-c3cccc4ccccc34)c2)c([2H])c([2H])c1-c1ccc2c(ccc3ccccc32)c1 ; [2H]c1c([2H])c(N(c2ccccc2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3ccccc3)c3cccc(-c4cccc5ccccc45)c3)c([2H])c2[2H])c([2H])c1[2H]]

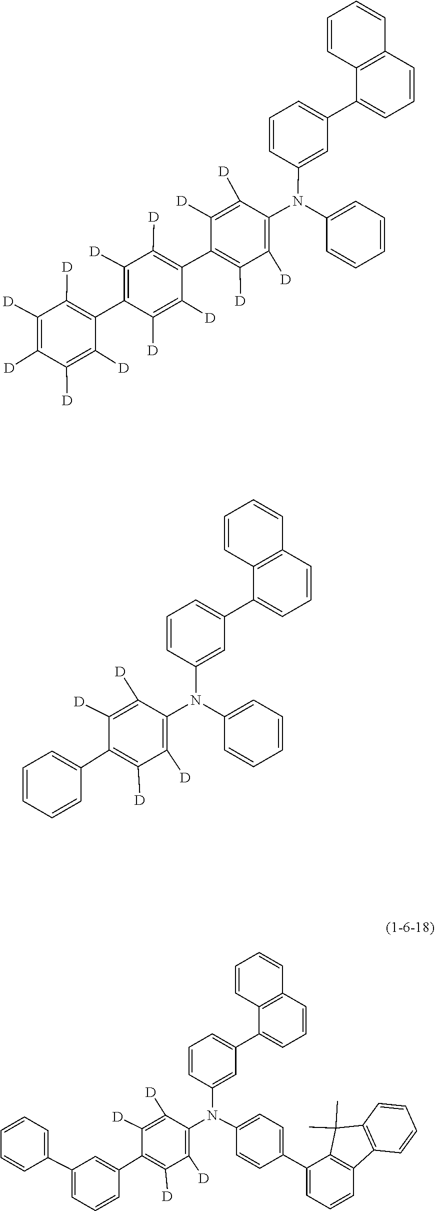 [SMILES: [2H]c1c([2H])c([2H])c(-c2c([2H])c([2H])c(-c3c([2H])c([2H])c(N(c4ccccc4)c4cccc(-c5cccc6ccccc56)c4)c([2H])c3[2H])c([2H])c2[2H])c([2H])c1[2H] ; [2H]c1c([2H])c(N(c2ccccc2)c2cccc(-c3cccc4ccccc34)c2)c([2H])c([2H])c1-c1ccccc1 ; [2H]c1c([2H])c(N(c2ccc(-c3cccc4c3C(C)(C)c3ccccc3-4)cc2)c2cccc(-c3cccc4ccccc34)c2)c([2H])c([2H])c1-c1cccc(-c2ccccc2)c1]

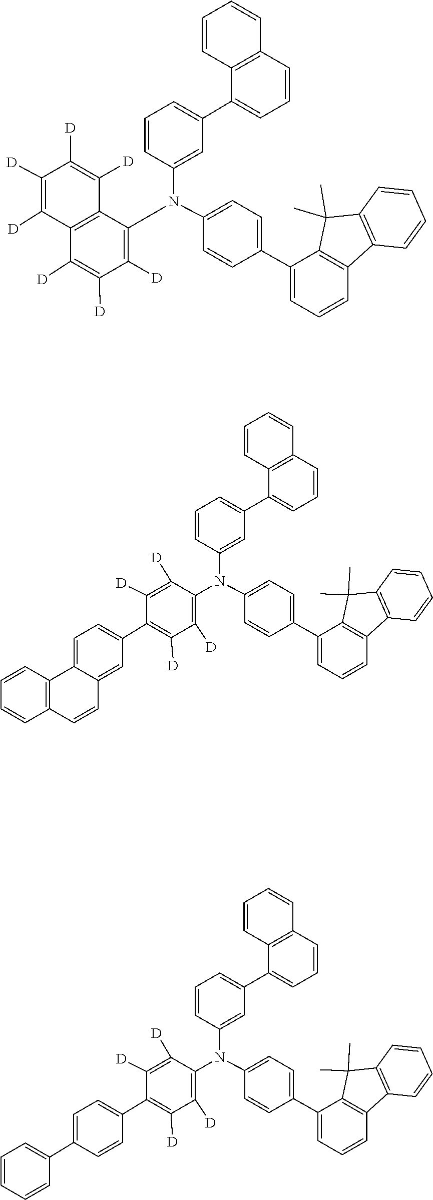 [SMILES: [2H]c1c([2H])c([2H])c2c(N(c3ccc(-c4cccc5c4C(C)(C)c4ccccc4-5)cc3)c3cccc(-c4cccc5ccccc45)c3)c([2H])c([2H])c([2H])c2c1[2H] ; [2H]c1c([2H])c(N(c2ccc(-c3cccc4c3C(C)(C)c3ccccc3-4)cc2)c2cccc(-c3cccc4ccccc34)c2)c([2H])c([2H])c1-c1ccc2c(ccc3ccccc32)c1 ; [2H]c1c([2H])c(N(c2ccc(-c3cccc4c3C(C)(C)c3ccccc3-4)cc2)c2cccc(-c3cccc4ccccc34)c2)c([2H])c([2H])c1-c1ccc(-c2ccccc2)cc1]

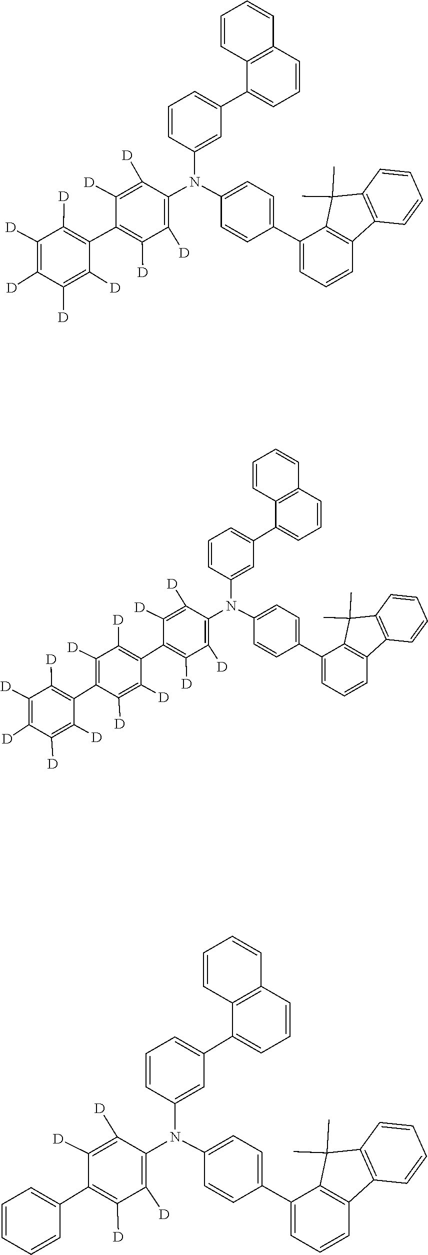 [SMILES: [2H]c1c([2H])c([2H])c(-c2c([2H])c([2H])c(N(c3ccc(-c4cccc5c4C(C)(C)c4ccccc4-5)cc3)c3cccc(-c4cccc5ccccc45)c3)c([2H])c2[2H])c([2H])c1[2H] ; [2H]c1c([2H])c([2H])c(-c2c([2H])c([2H])c(-c3c([2H])c([2H])c(N(c4ccc(-c5cccc6c5C(C)(C)c5ccccc5-6)cc4)c4cccc(-c5cccc6ccccc56)c4)c([2H])c3[2H])c([2H])c2[2H])c([2H])c1[2H] ; [2H]c1c([2H])c(N(c2ccc(-c3cccc4c3C(C)(C)c3ccccc3-4)cc2)c2cccc(-c3cccc4ccccc34)c2)c([2H])c([2H])c1-c1ccccc1]

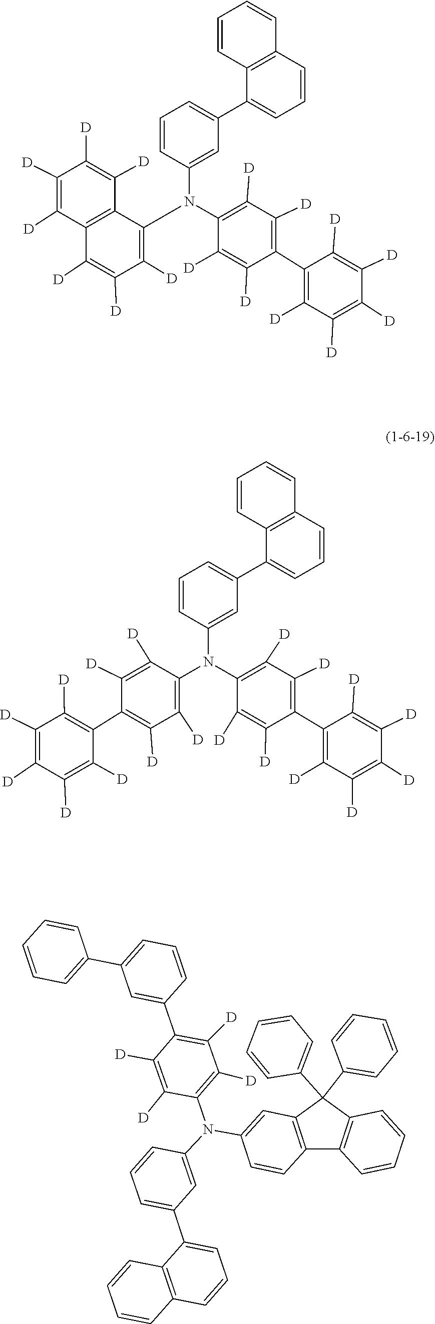 [SMILES: [2H]c1c([2H])c([2H])c(-c2c([2H])c([2H])c(N(c3cccc(-c4cccc5ccccc45)c3)c3c([2H])c([2H])c([2H])c4c([2H])c([2H])c([2H])c([2H])c34)c([2H])c2[2H])c([2H])c1[2H] ; [2H]c1c([2H])c([2H])c(-c2c([2H])c([2H])c(N(c3cccc(-c4cccc5ccccc45)c3)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c(N(c2cccc(-c3cccc4ccccc34)c2)c2ccc3c(c2)C(c2ccccc2)(c2ccccc2)c2ccccc2-3)c([2H])c([2H])c1-c1cccc(-c2ccccc2)c1]

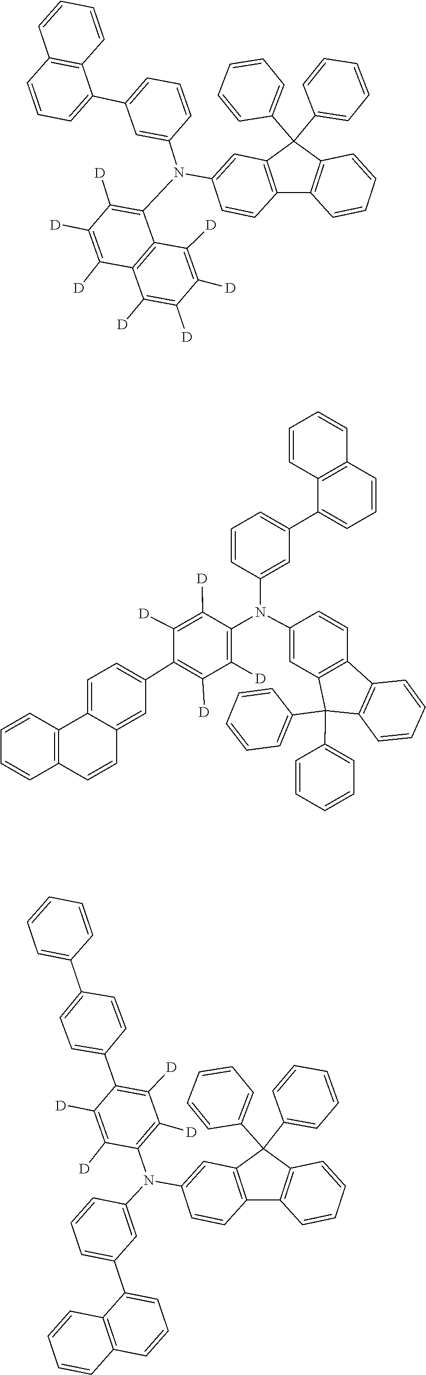 [SMILES: [2H]c1c([2H])c([2H])c2c(N(c3cccc(-c4cccc5ccccc45)c3)c3ccc4c(c3)C(c3ccccc3)(c3ccccc3)c3ccccc3-4)c([2H])c([2H])c([2H])c2c1[2H] ; [2H]c1c([2H])c(N(c2cccc(-c3cccc4ccccc34)c2)c2ccc3c(c2)C(c2ccccc2)(c2ccccc2)c2ccccc2-3)c([2H])c([2H])c1-c1ccc2c(ccc3ccccc32)c1 ; [2H]c1c([2H])c(N(c2cccc(-c3cccc4ccccc34)c2)c2ccc3c(c2)C(c2ccccc2)(c2ccccc2)c2ccccc2-3)c([2H])c([2H])c1-c1ccc(-c2ccccc2)cc1]

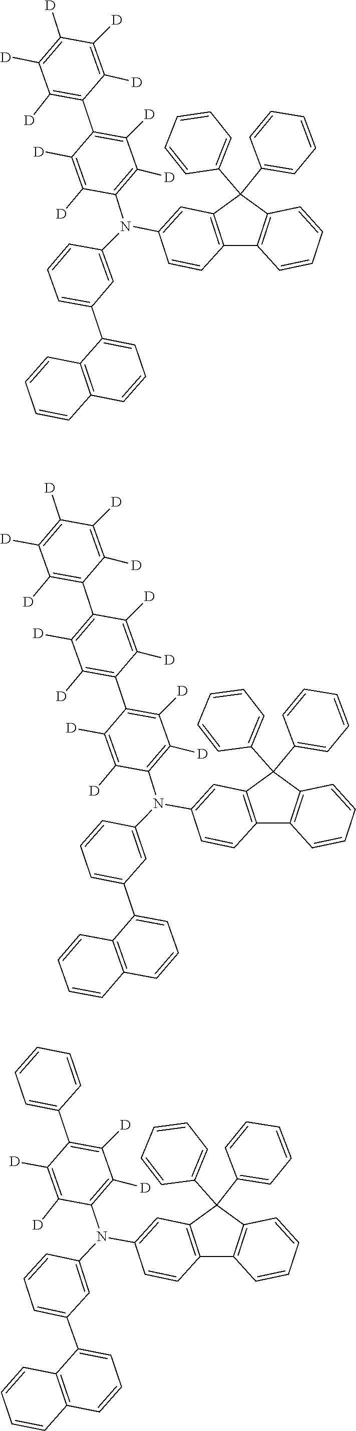 [SMILES: [2H]c1c([2H])c([2H])c(-c2c([2H])c([2H])c(N(c3cccc(-c4cccc5ccccc45)c3)c3ccc4c(c3)C(c3ccccc3)(c3ccccc3)c3ccccc3-4)c([2H])c2[2H])c([2H])c1[2H] ; [2H]c1c([2H])c([2H])c(-c2c([2H])c([2H])c(-c3c([2H])c([2H])c(N(c4cccc(-c5cccc6ccccc56)c4)c4ccc5c(c4)C(c4ccccc4)(c4ccccc4)c4ccccc4-5)c([2H])c3[2H])c([2H])c2[2H])c([2H])c1[2H] ; [2H]c1c([2H])c(N(c2cccc(-c3cccc4ccccc34)c2)c2ccc3c(c2)C(c2ccccc2)(c2ccccc2)c2ccccc2-3)c([2H])c([2H])c1-c1ccccc1]

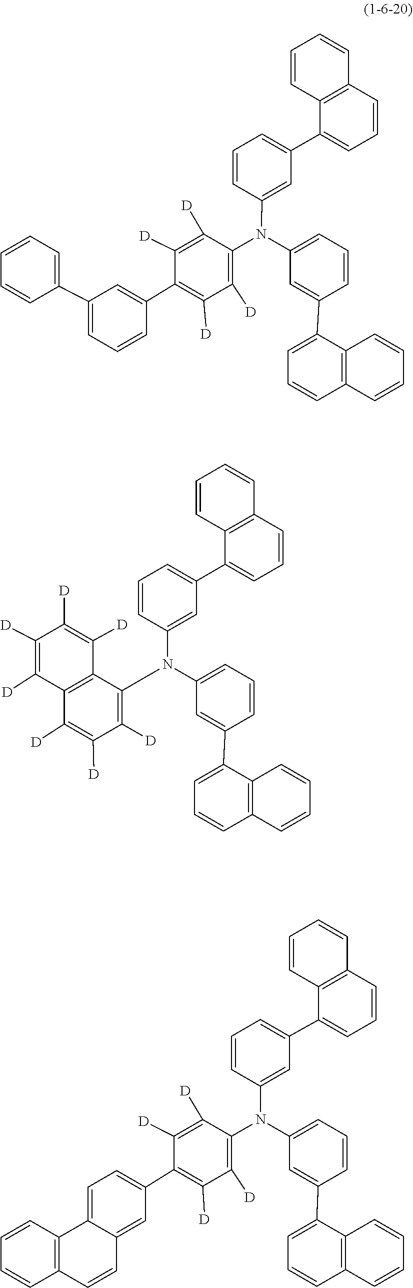 [SMILES: [2H]c1c([2H])c(N(c2cccc(-c3cccc4ccccc34)c2)c2cccc(-c3cccc4ccccc34)c2)c([2H])c([2H])c1-c1cccc(-c2ccccc2)c1 ; [2H]c1c([2H])c([2H])c2c(N(c3cccc(-c4cccc5ccccc45)c3)c3cccc(-c4cccc5ccccc45)c3)c([2H])c([2H])c([2H])c2c1[2H] ; [2H]c1c([2H])c(N(c2cccc(-c3cccc4ccccc34)c2)c2cccc(-c3cccc4ccccc34)c2)c([2H])c([2H])c1-c1ccc2c(ccc3ccccc32)c1]

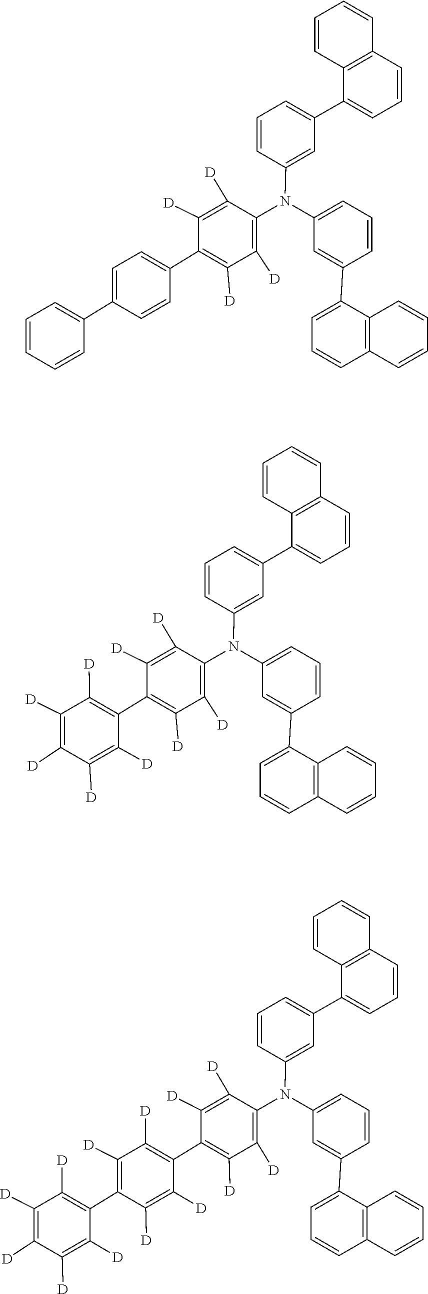 [SMILES: [2H]c1c([2H])c(N(c2cccc(-c3cccc4ccccc34)c2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3cccc(-c4cccc5ccccc45)c3)c3cccc(-c4cccc5ccccc45)c3)c([2H])c2[2H])c([2H])c1[2H] ; [2H]c1c([2H])c([2H])c(-c2c([2H])c([2H])c(-c3c([2H])c([2H])c(N(c4cccc(-c5cccc6ccccc56)c4)c4cccc(-c5cccc6ccccc56)c4)c([2H])c3[2H])c([2H])c2[2H])c([2H])c1[2H]]

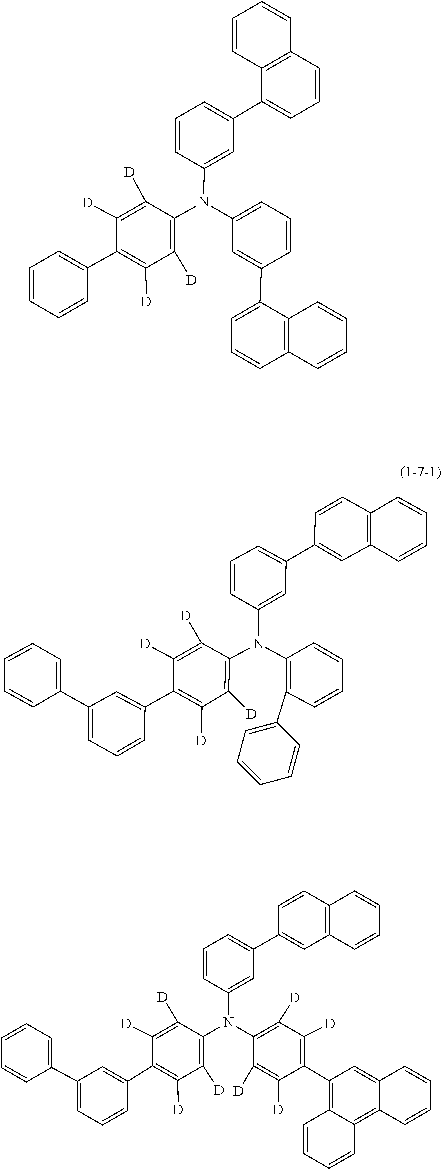 [SMILES: [2H]c1c([2H])c(N(c2cccc(-c3cccc4ccccc34)c2)c2cccc(-c3cccc4ccccc34)c2)c([2H])c([2H])c1-c1ccccc1 ; [2H]c1c([2H])c(N(c2cccc(-c3ccc4ccccc4c3)c2)c2ccccc2-c2ccccc2)c([2H])c([2H])c1-c1cccc(-c2ccccc2)c1 ; [2H]c1c([2H])c(N(c2cccc(-c3ccc4ccccc4c3)c2)c2c([2H])c([2H])c(-c3cc4ccccc4c4ccccc34)c([2H])c2[2H])c([2H])c([2H])c1-c1cccc(-c2ccccc2)c1]

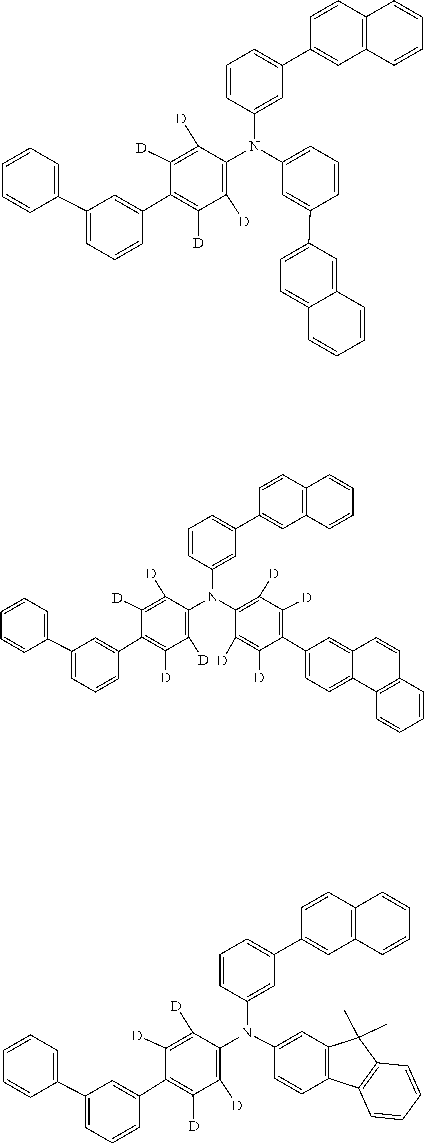 [SMILES: [2H]c1c([2H])c(N(c2cccc(-c3ccc4ccccc4c3)c2)c2cccc(-c3ccc4ccccc4c3)c2)c([2H])c([2H])c1-c1cccc(-c2ccccc2)c1 ; [2H]c1c([2H])c(N(c2cccc(-c3ccc4ccccc4c3)c2)c2c([2H])c([2H])c(-c3ccc4c(ccc5ccccc54)c3)c([2H])c2[2H])c([2H])c([2H])c1-c1cccc(-c2ccccc2)c1 ; [2H]c1c([2H])c(N(c2cccc(-c3ccc4ccccc4c3)c2)c2ccc3c(c2)C(C)(C)c2ccccc2-3)c([2H])c([2H])c1-c1cccc(-c2ccccc2)c1]

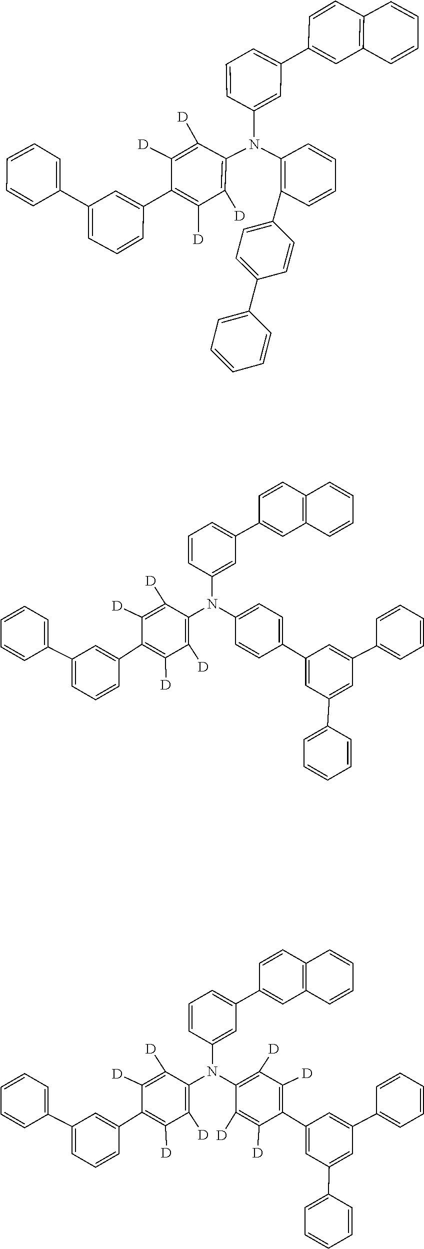 [SMILES: [2H]c1c([2H])c(N(c2cccc(-c3ccc4ccccc4c3)c2)c2ccccc2-c2ccc(-c3ccccc3)cc2)c([2H])c([2H])c1-c1cccc(-c2ccccc2)c1 ; [2H]c1c([2H])c(N(c2ccc(-c3cc(-c4ccccc4)cc(-c4ccccc4)c3)cc2)c2cccc(-c3ccc4ccccc4c3)c2)c([2H])c([2H])c1-c1cccc(-c2ccccc2)c1 ; [2H]c1c([2H])c(N(c2cccc(-c3ccc4ccccc4c3)c2)c2c([2H])c([2H])c(-c3cc(-c4ccccc4)cc(-c4ccccc4)c3)c([2H])c2[2H])c([2H])c([2H])c1-c1cccc(-c2ccccc2)c1]

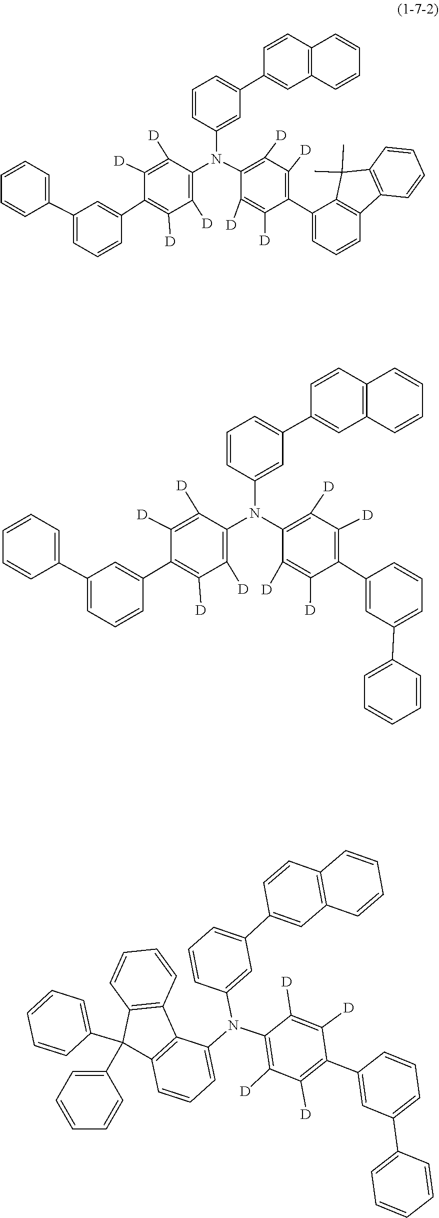 [SMILES: [2H]c1c([2H])c(N(c2cccc(-c3ccc4ccccc4c3)c2)c2c([2H])c([2H])c(-c3cccc4c3C(C)(C)c3ccccc3-4)c([2H])c2[2H])c([2H])c([2H])c1-c1cccc(-c2ccccc2)c1 ; [2H]c1c([2H])c(N(c2cccc(-c3ccc4ccccc4c3)c2)c2c([2H])c([2H])c(-c3cccc(-c4ccccc4)c3)c([2H])c2[2H])c([2H])c([2H])c1-c1cccc(-c2ccccc2)c1 ; [2H]c1c([2H])c(N(c2cccc(-c3ccc4ccccc4c3)c2)c2cccc3c2-c2ccccc2C3(c2ccccc2)c2ccccc2)c([2H])c([2H])c1-c1cccc(-c2ccccc2)c1]

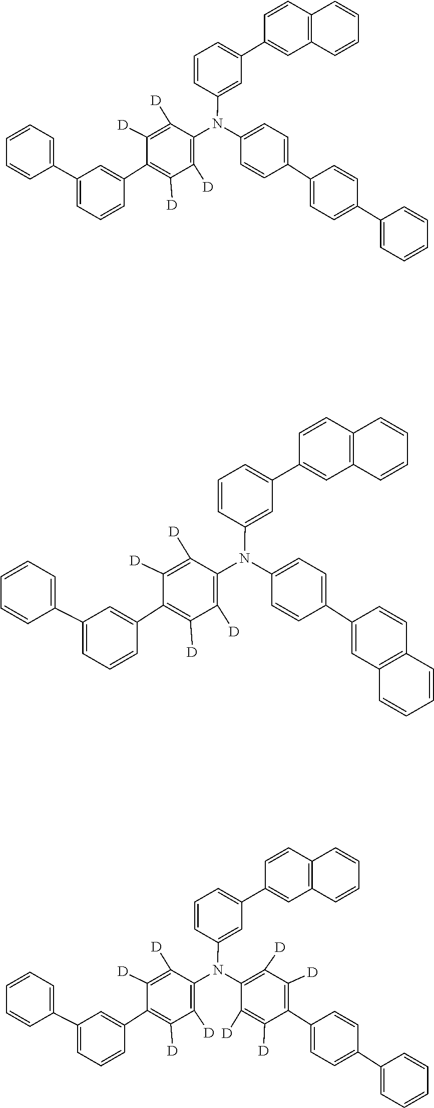 [SMILES: [2H]c1c([2H])c(N(c2ccc(-c3ccc(-c4ccccc4)cc3)cc2)c2cccc(-c3ccc4ccccc4c3)c2)c([2H])c([2H])c1-c1cccc(-c2ccccc2)c1 ; [2H]c1c([2H])c(N(c2ccc(-c3ccc4ccccc4c3)cc2)c2cccc(-c3ccc4ccccc4c3)c2)c([2H])c([2H])c1-c1cccc(-c2ccccc2)c1 ; [2H]c1c([2H])c(N(c2cccc(-c3ccc4ccccc4c3)c2)c2c([2H])c([2H])c(-c3cccc(-c4ccccc4)c3)c([2H])c2[2H])c([2H])c([2H])c1-c1ccc(-c2ccccc2)cc1]

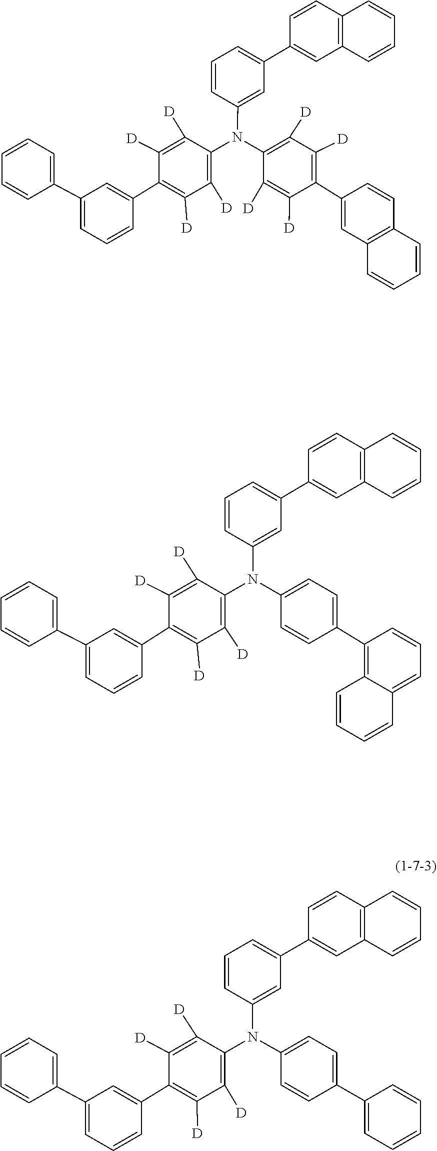 [SMILES: [2H]c1c([2H])c(N(c2cccc(-c3ccc4ccccc4c3)c2)c2c([2H])c([2H])c(-c3ccc4ccccc4c3)c([2H])c2[2H])c([2H])c([2H])c1-c1cccc(-c2ccccc2)c1 ; [2H]c1c([2H])c(N(c2ccc(-c3cccc4ccccc34)cc2)c2cccc(-c3ccc4ccccc4c3)c2)c([2H])c([2H])c1-c1cccc(-c2ccccc2)c1 ; [2H]c1c([2H])c(N(c2ccc(-c3ccccc3)cc2)c2cccc(-c3ccc4ccccc4c3)c2)c([2H])c([2H])c1-c1cccc(-c2ccccc2)c1]

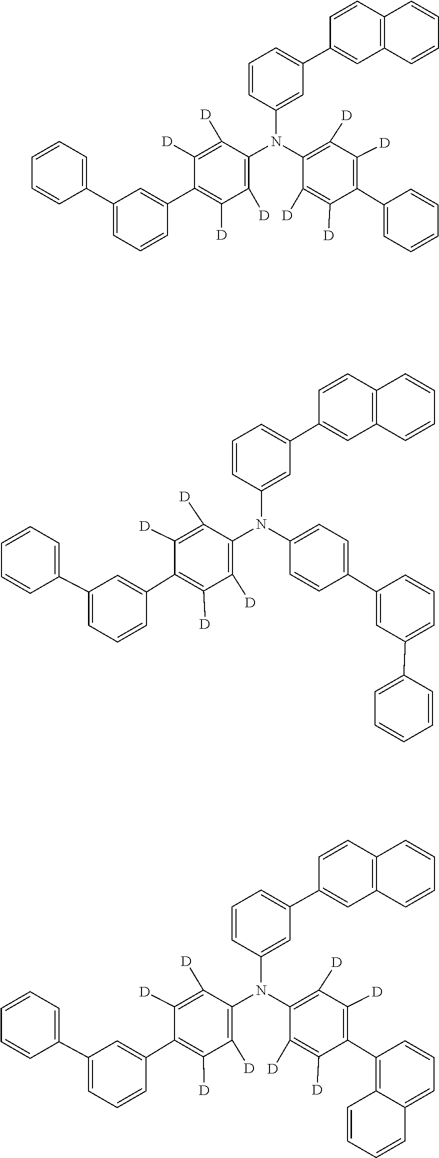 [SMILES: [2H]c1c([2H])c(N(c2cccc(-c3ccc4ccccc4c3)c2)c2c([2H])c([2H])c(-c3cccc(-c4ccccc4)c3)c([2H])c2[2H])c([2H])c([2H])c1-c1ccccc1 ; [2H]c1c([2H])c(N(c2ccc(-c3cccc(-c4ccccc4)c3)cc2)c2cccc(-c3ccc4ccccc4c3)c2)c([2H])c([2H])c1-c1cccc(-c2ccccc2)c1 ; [2H]c1c([2H])c(N(c2cccc(-c3ccc4ccccc4c3)c2)c2c([2H])c([2H])c(-c3cccc4ccccc34)c([2H])c2[2H])c([2H])c([2H])c1-c1cccc(-c2ccccc2)c1]

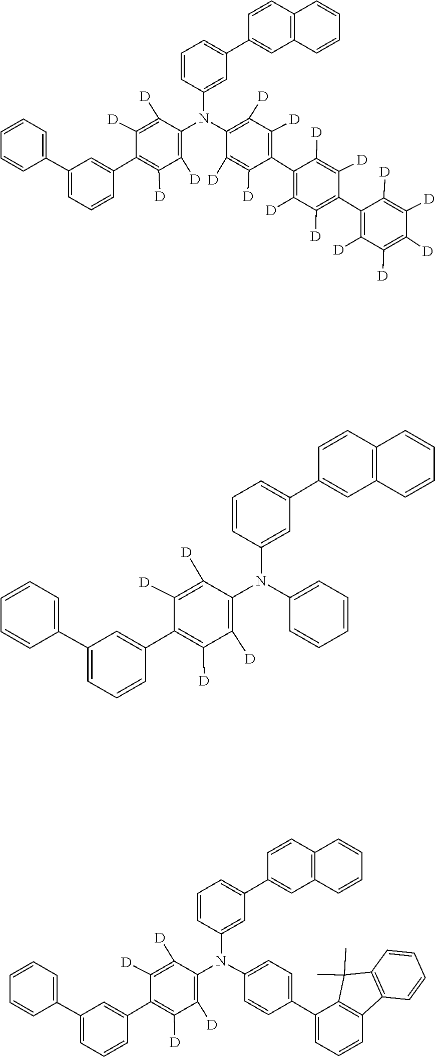 [SMILES: [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5cccc(-c6ccccc6)c5)c([2H])c4[2H])c([2H])c3[2H])c([2H])c2[2H])c([2H])c1[2H] ; [2H]c1c([2H])c(N(c2ccccc2)c2cccc(-c3ccc4ccccc4c3)c2)c([2H])c([2H])c1-c1cccc(-c2ccccc2)c1 ; [2H]c1c([2H])c(N(c2ccc(-c3cccc4c3C(C)(C)c3ccccc3-4)cc2)c2cccc(-c3ccc4ccccc4c3)c2)c([2H])c([2H])c1-c1cccc(-c2ccccc2)c1]

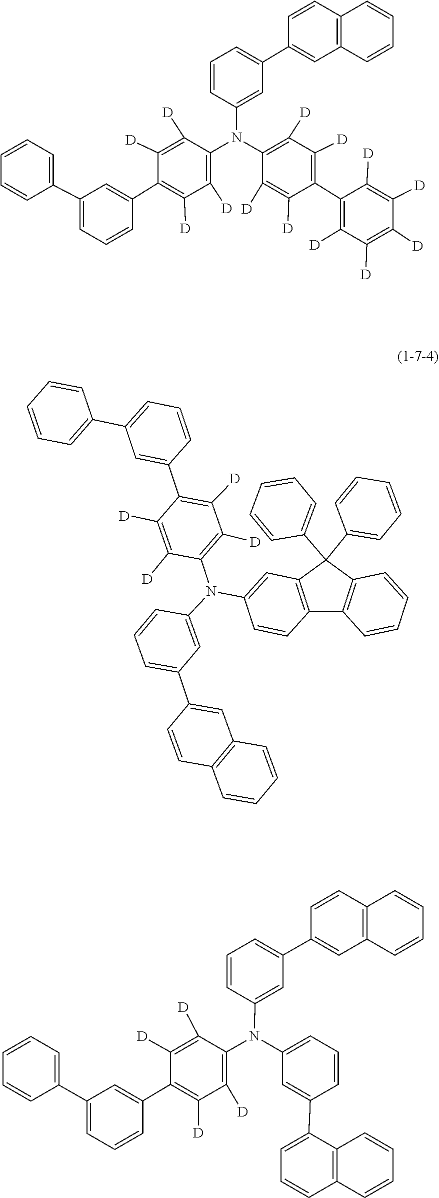 [SMILES: [2H]c1c([2H])c([2H])c(-c2c([2H])c([2H])c(N(c3cccc(-c4ccc5ccccc5c4)c3)c3c([2H])c([2H])c(-c4cccc(-c5ccccc5)c4)c([2H])c3[2H])c([2H])c2[2H])c([2H])c1[2H] ; [2H]c1c([2H])c(N(c2cccc(-c3ccc4ccccc4c3)c2)c2ccc3c(c2)C(c2ccccc2)(c2ccccc2)c2ccccc2-3)c([2H])c([2H])c1-c1cccc(-c2ccccc2)c1 ; [2H]c1c([2H])c(N(c2cccc(-c3ccc4ccccc4c3)c2)c2cccc(-c3cccc4ccccc34)c2)c([2H])c([2H])c1-c1cccc(-c2ccccc2)c1]

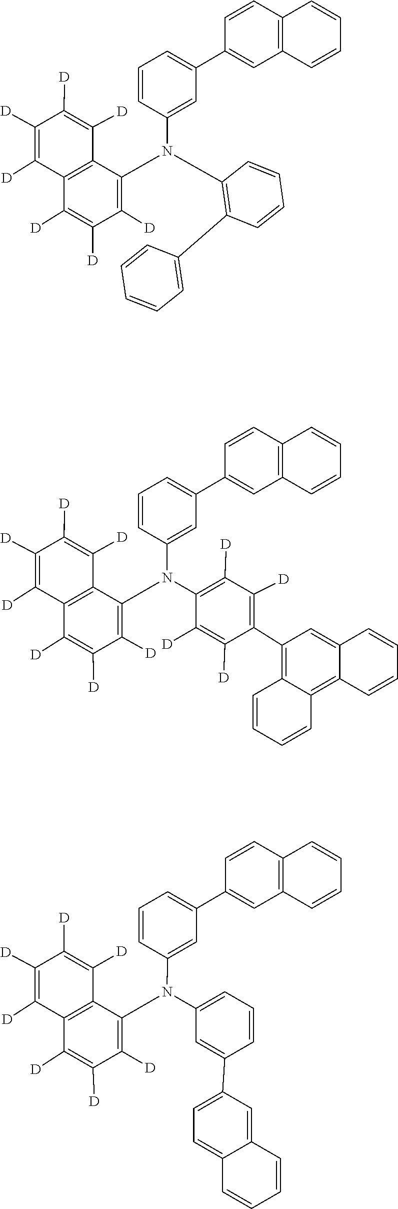 [SMILES: [2H]c1c([2H])c([2H])c2c(N(c3cccc(-c4ccc5ccccc5c4)c3)c3ccccc3-c3ccccc3)c([2H])c([2H])c([2H])c2c1[2H] ; [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1cc2ccccc2c2ccccc12 ; [2H]c1c([2H])c([2H])c2c(N(c3cccc(-c4ccc5ccccc5c4)c3)c3cccc(-c4ccc5ccccc5c4)c3)c([2H])c([2H])c([2H])c2c1[2H]]

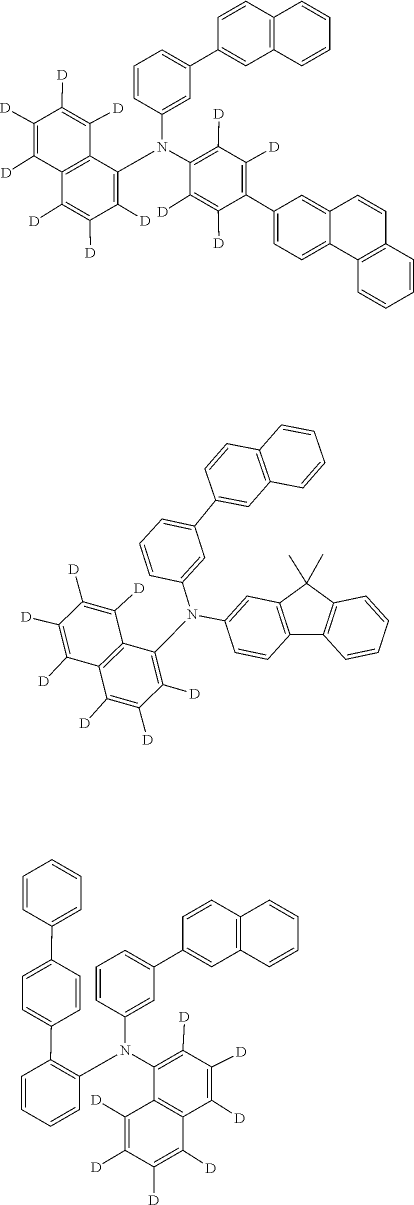 [SMILES: [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1ccc2c(ccc3ccccc32)c1 ; [2H]c1c([2H])c([2H])c2c(N(c3cccc(-c4ccc5ccccc5c4)c3)c3ccc4c(c3)C(C)(C)c3ccccc3-4)c([2H])c([2H])c([2H])c2c1[2H] ; [2H]c1c([2H])c([2H])c2c(N(c3cccc(-c4ccc5ccccc5c4)c3)c3ccccc3-c3ccc(-c4ccccc4)cc3)c([2H])c([2H])c([2H])c2c1[2H]]

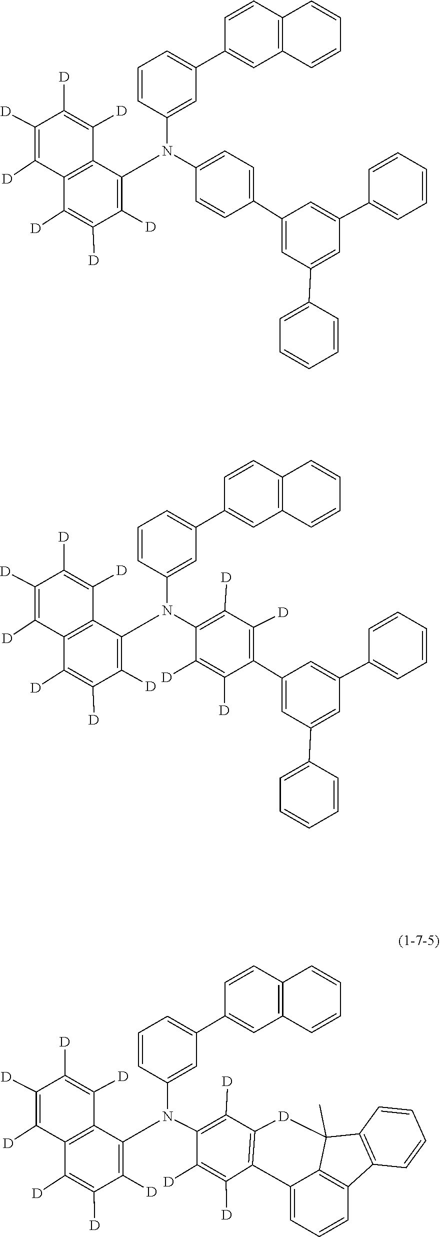

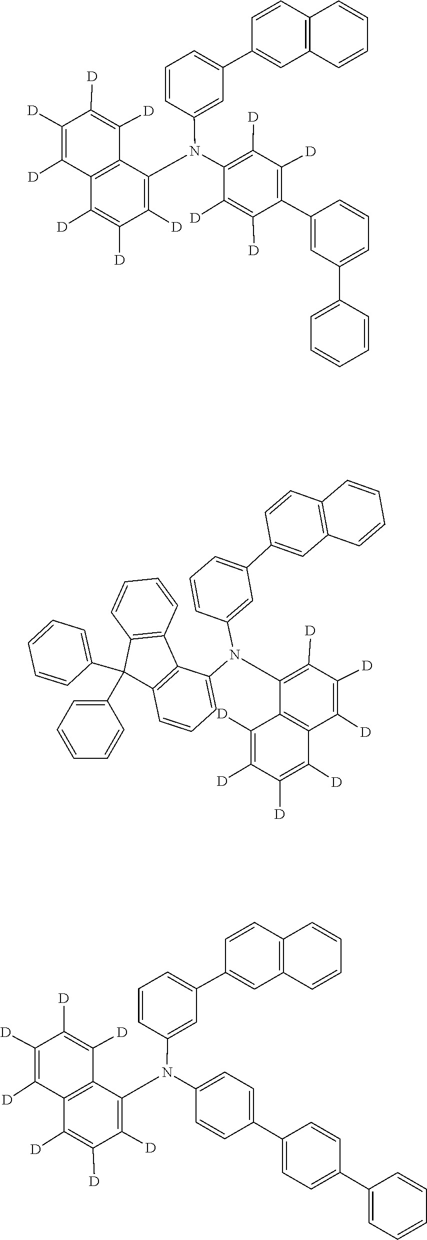 [SMILES: [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1cccc(-c2ccccc2)c1 ; [2H]c1c([2H])c([2H])c2c(N(c3cccc(-c4ccc5ccccc5c4)c3)c3cccc4c3-c3ccccc3C4(c3ccccc3)c3ccccc3)c([2H])c([2H])c([2H])c2c1[2H] ; [2H]c1c([2H])c([2H])c2c(N(c3ccc(-c4ccc(-c5ccccc5)cc4)cc3)c3cccc(-c4ccc5ccccc5c4)c3)c([2H])c([2H])c([2H])c2c1[2H]]

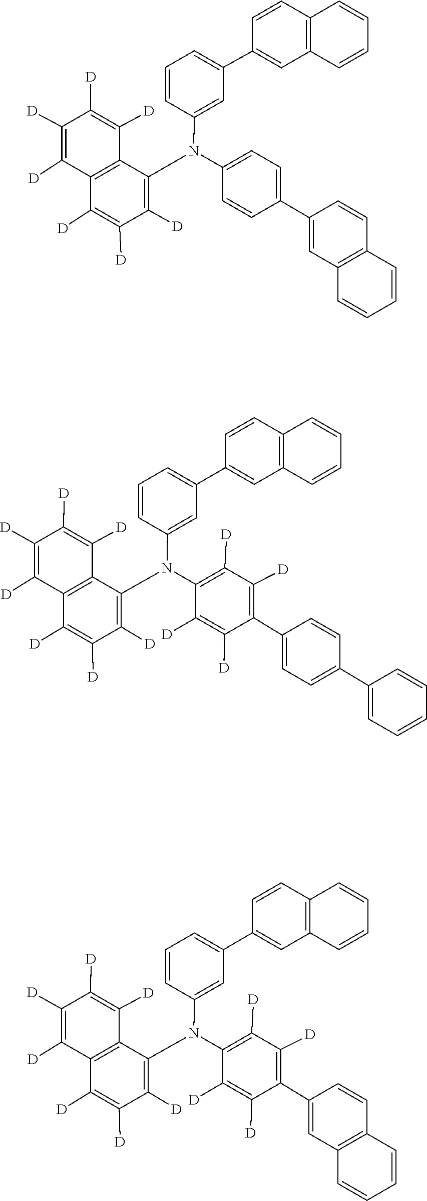 [SMILES: [2H]c1c([2H])c([2H])c2c(N(c3ccc(-c4ccc5ccccc5c4)cc3)c3cccc(-c4ccc5ccccc5c4)c3)c([2H])c([2H])c([2H])c2c1[2H] ; [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1ccc(-c2ccccc2)cc1 ; [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1ccc2ccccc2c1]

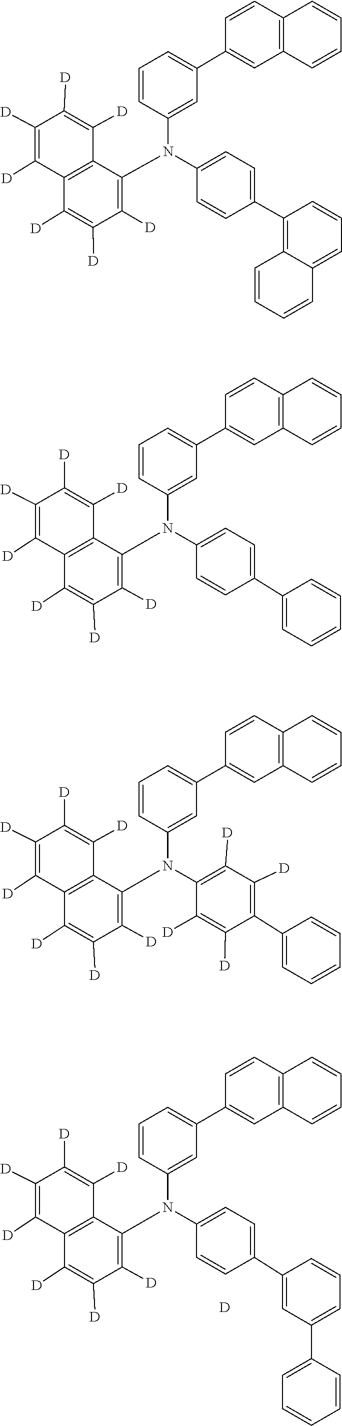

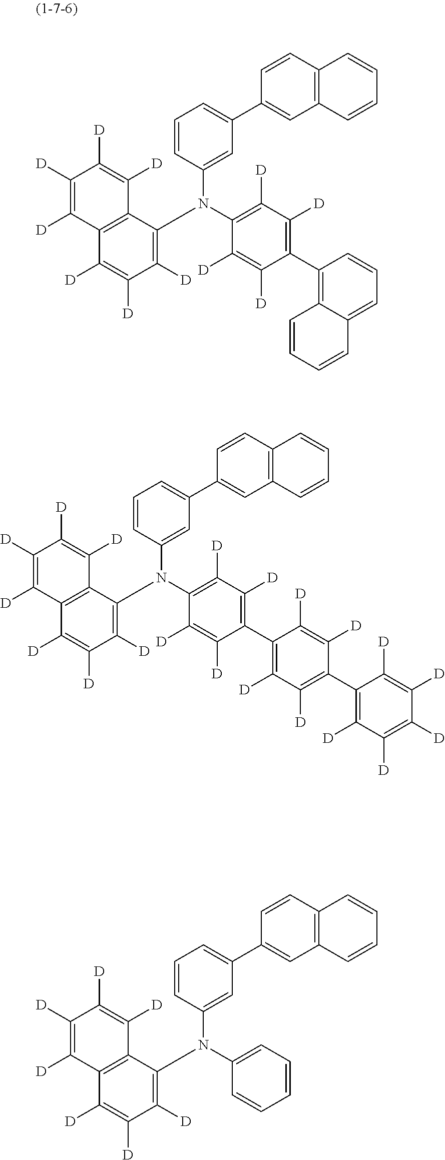 [SMILES: [2H]c1c([2H])c(N(c2cccc(-c3ccc4ccccc4c3)c2)c2c([2H])c([2H])c([2H])c3c([2H])c([2H])c([2H])c([2H])c23)c([2H])c([2H])c1-c1cccc2ccccc12 ; [2H]c1c([2H])c([2H])c(-c2c([2H])c([2H])c(-c3c([2H])c([2H])c(N(c4cccc(-c5ccc6ccccc6c5)c4)c4c([2H])c([2H])c([2H])c5c([2H])c([2H])c([2H])c([2H])c45)c([2H])c3[2H])c([2H])c2[2H])c([2H])c1[2H] ; [2H]c1c([2H])c([2H])c2c(N(c3ccccc3)c3cccc(-c4ccc5ccccc5c4)c3)c([2H])c([2H])c([2H])c2c1[2H]]

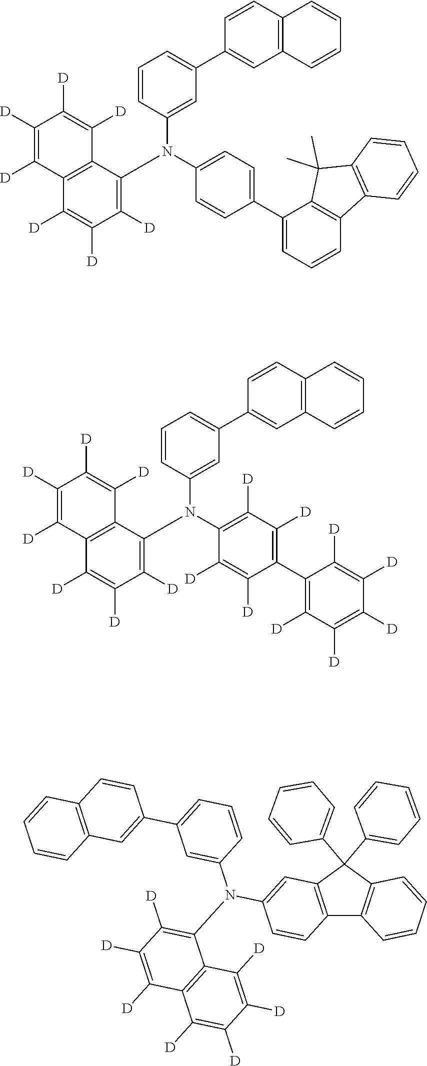 [SMILES: [2H]c1c([2H])c([2H])c2c(N(c3ccc(-c4cccc5c4C(C)(C)c4ccccc4-5)cc3)c3cccc(-c4ccc5ccccc5c4)c3)c([2H])c([2H])c([2H])c2c1[2H] ; [2H]c1c([2H])c([2H])c(-c2c([2H])c([2H])c(N(c3cccc(-c4ccc5ccccc5c4)c3)c3c([2H])c([2H])c([2H])c4c([2H])c([2H])c([2H])c([2H])c34)c([2H])c2[2H])c([2H])c1[2H] ; [2H]c1c([2H])c([2H])c2c(N(c3cccc(-c4ccc5ccccc5c4)c3)c3ccc4c(c3)C(c3ccccc3)(c3ccccc3)c3ccccc3-4)c([2H])c([2H])c([2H])c2c1[2H]]

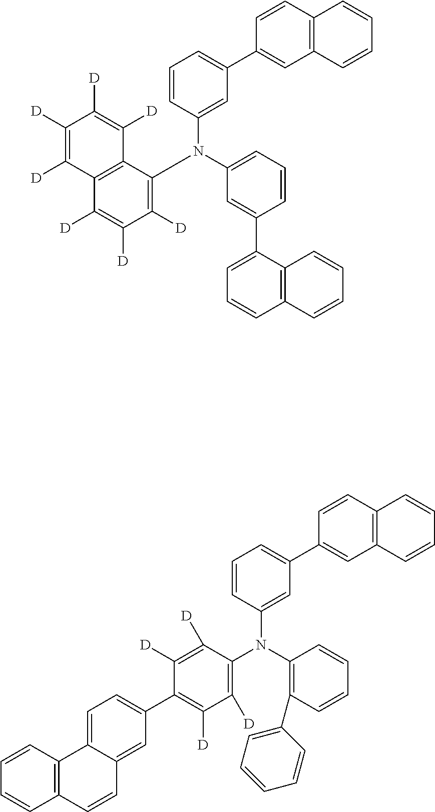 [SMILES: [2H]c1c([2H])c([2H])c2c(N(c3cccc(-c4ccc5ccccc5c4)c3)c3cccc(-c4cccc5ccccc45)c3)c([2H])c([2H])c([2H])c2c1[2H] ; [2H]c1c([2H])c(N(c2cccc(-c3ccc4ccccc4c3)c2)c2ccccc2-c2ccccc2)c([2H])c([2H])c1-c1ccc2c(ccc3ccccc32)c1]

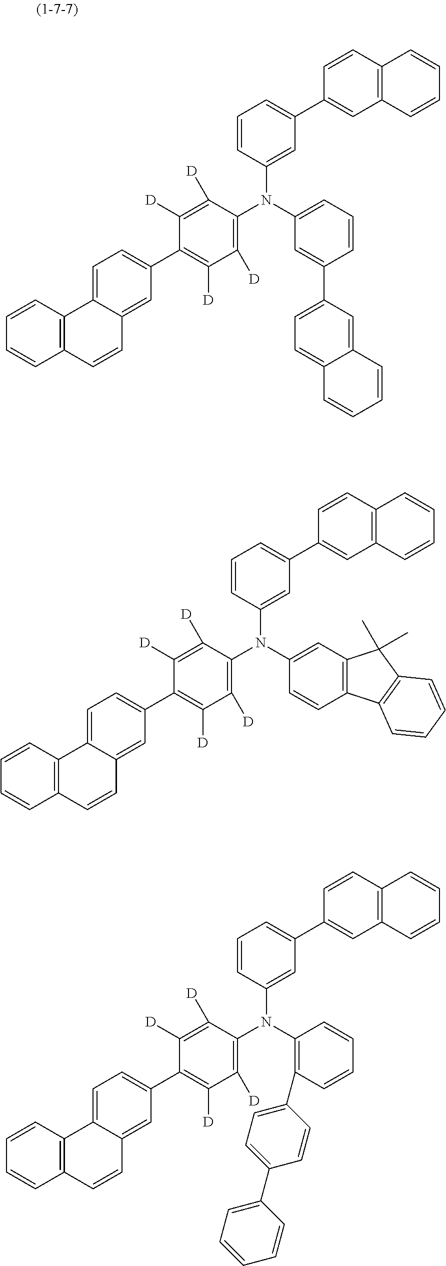 [SMILES: [2H]c1c([2H])c(N(c2cccc(-c3ccc4ccccc4c3)c2)c2cccc(-c3ccc4ccccc4c3)c2)c([2H])c([2H])c1-c1ccc2c(ccc3ccccc32)c1 ; [2H]c1c([2H])c(N(c2cccc(-c3ccc4ccccc4c3)c2)c2ccc3c(c2)C(C)(C)c2ccccc2-3)c([2H])c([2H])c1-c1ccc2c(ccc3ccccc32)c1 ; [2H]c1c([2H])c(N(c2cccc(-c3ccc4ccccc4c3)c2)c2ccccc2-c2ccc(-c3ccccc3)cc2)c([2H])c([2H])c1-c1ccc2c(ccc3ccccc32)c1]

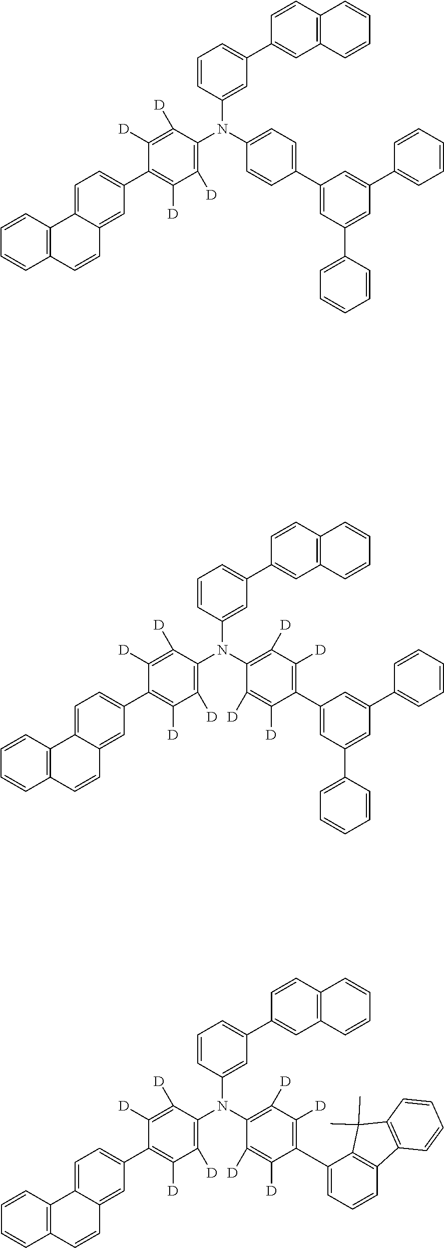 [SMILES: [2H]c1c([2H])c(N(c2ccc(-c3cc(-c4ccccc4)cc(-c4ccccc4)c3)cc2)c2cccc(-c3ccc4ccccc4c3)c2)c([2H])c([2H])c1-c1ccc2c(ccc3ccccc32)c1 ; [2H]c1c([2H])c(N(c2cccc(-c3ccc4ccccc4c3)c2)c2c([2H])c([2H])c(-c3ccc4c(ccc5ccccc54)c3)c([2H])c2[2H])c([2H])c([2H])c1-c1cc(-c2ccccc2)cc(-c2ccccc2)c1 ; [2H]c1c([2H])c(N(c2cccc(-c3ccc4ccccc4c3)c2)c2c([2H])c([2H])c(-c3cccc4c3C(C)(C)c3ccccc3-4)c([2H])c2[2H])c([2H])c([2H])c1-c1ccc2c(ccc3ccccc32)c1]

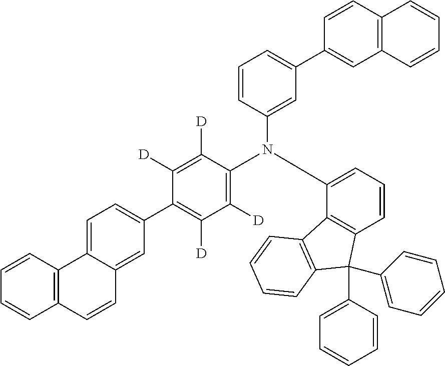 [SMILES: [2H]c1c([2H])c(N(c2cccc(-c3ccc4ccccc4c3)c2)c2cccc3c2-c2ccccc2C3(c2ccccc2)c2ccccc2)c([2H])c([2H])c1-c1ccc2c(ccc3ccccc32)c1]

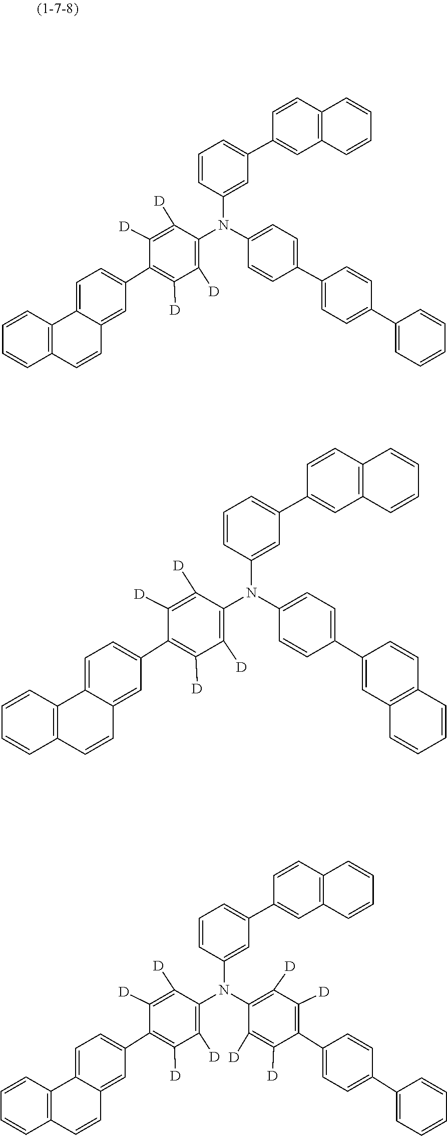 [SMILES: [2H]c1c([2H])c(N(c2ccc(-c3ccc(-c4ccccc4)cc3)cc2)c2cccc(-c3ccc4ccccc4c3)c2)c([2H])c([2H])c1-c1ccc2c(ccc3ccccc32)c1 ; [2H]c1c([2H])c(N(c2ccc(-c3ccc4ccccc4c3)cc2)c2cccc(-c3ccc4ccccc4c3)c2)c([2H])c([2H])c1-c1ccc2c(ccc3ccccc32)c1 ; [2H]c1c([2H])c(N(c2cccc(-c3ccc4ccccc4c3)c2)c2c([2H])c([2H])c(-c3ccc4c(ccc5ccccc54)c3)c([2H])c2[2H])c([2H])c([2H])c1-c1ccc(-c2ccccc2)cc1]

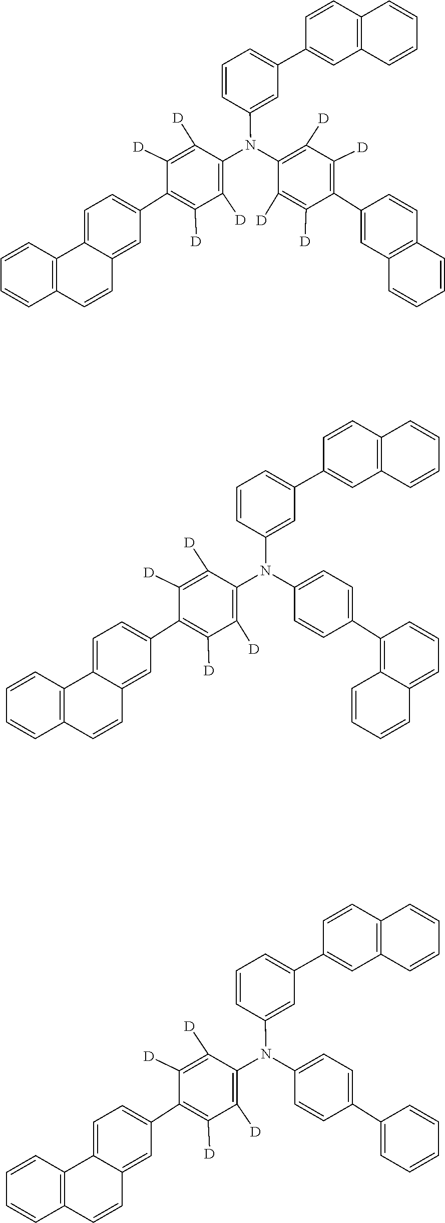 [SMILES: [2H]c1c([2H])c(N(c2cccc(-c3ccc4ccccc4c3)c2)c2c([2H])c([2H])c(-c3ccc4c(ccc5ccccc54)c3)c([2H])c2[2H])c([2H])c([2H])c1-c1ccc2ccccc2c1 ; [2H]c1c([2H])c(N(c2ccc(-c3cccc4ccccc34)cc2)c2cccc(-c3ccc4ccccc4c3)c2)c([2H])c([2H])c1-c1ccc2c(ccc3ccccc32)c1 ; [2H]c1c([2H])c(N(c2ccc(-c3ccccc3)cc2)c2cccc(-c3ccc4ccccc4c3)c2)c([2H])c([2H])c1-c1ccc2c(ccc3ccccc32)c1]

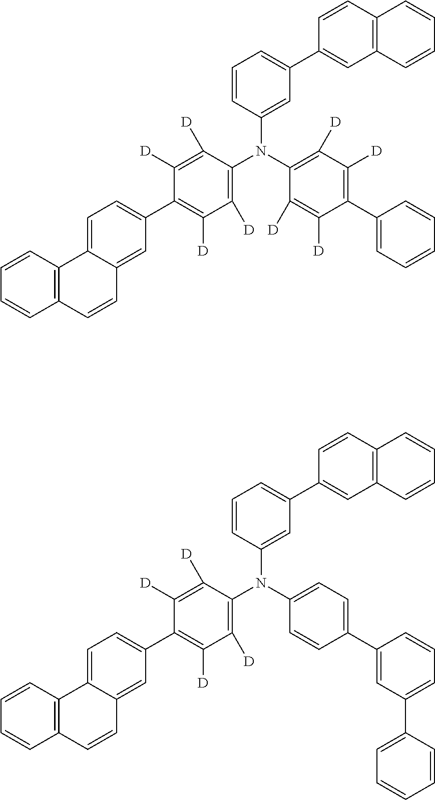 [SMILES: [2H]c1c([2H])c(N(c2cccc(-c3ccc4ccccc4c3)c2)c2c([2H])c([2H])c(-c3ccc4c(ccc5ccccc54)c3)c([2H])c2[2H])c([2H])c([2H])c1-c1ccccc1 ; [2H]c1c([2H])c(N(c2ccc(-c3cccc(-c4ccccc4)c3)cc2)c2cccc(-c3ccc4ccccc4c3)c2)c([2H])c([2H])c1-c1ccc2c(ccc3ccccc32)c1]

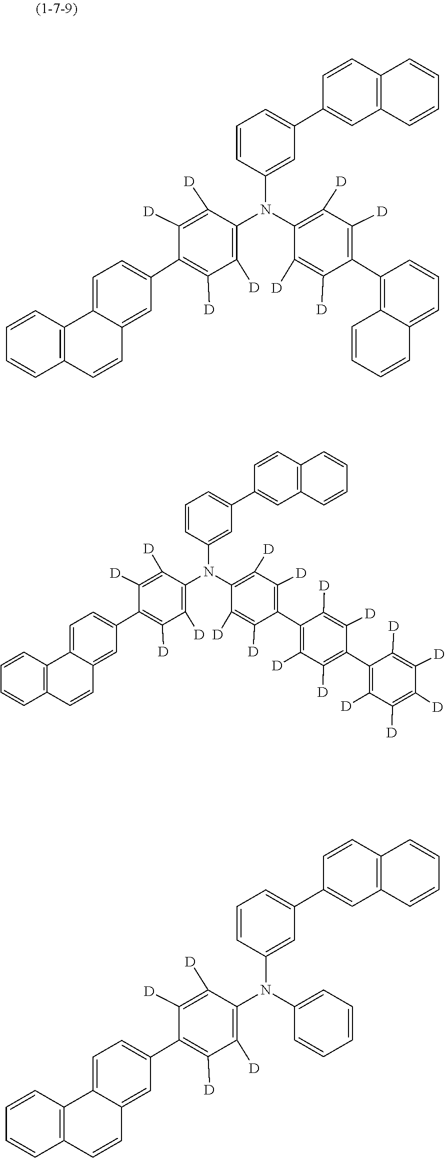 [SMILES: [2H]c1c([2H])c(N(c2cccc(-c3ccc4ccccc4c3)c2)c2c([2H])c([2H])c(-c3cccc4ccccc34)c([2H])c2[2H])c([2H])c([2H])c1-c1ccc2c(ccc3ccccc32)c1 ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5ccc6c(ccc7ccccc76)c5)c([2H])c4[2H])c([2H])c3[2H])c([2H])c2[2H])c([2H])c1[2H] ; [2H]c1c([2H])c(N(c2ccccc2)c2cccc(-c3ccc4ccccc4c3)c2)c([2H])c([2H])c1-c1ccc2c(ccc3ccccc32)c1]

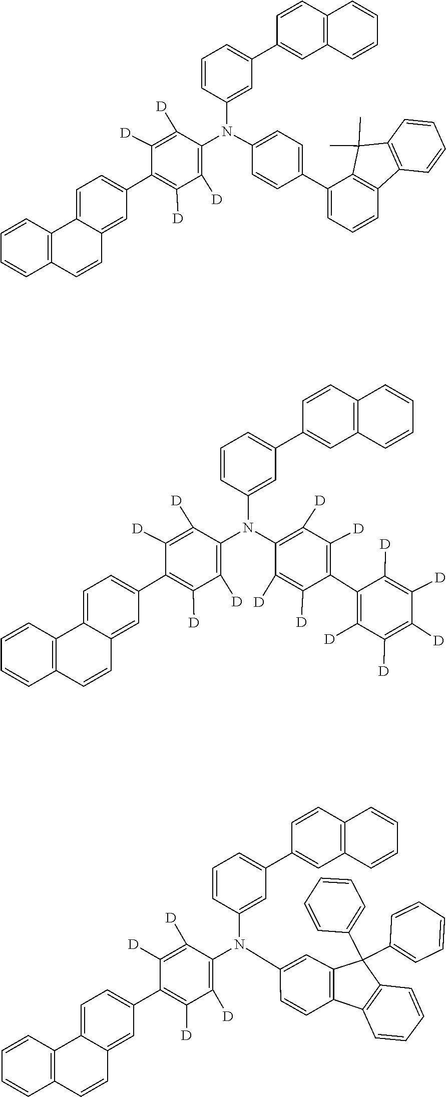 [SMILES: [2H]c1c([2H])c(N(c2ccc(-c3cccc4c3C(C)(C)c3ccccc3-4)cc2)c2cccc(-c3ccc4ccccc4c3)c2)c([2H])c([2H])c1-c1ccc2c(ccc3ccccc32)c1 ; [2H]c1c([2H])c([2H])c(-c2c([2H])c([2H])c(N(c3cccc(-c4ccc5ccccc5c4)c3)c3c([2H])c([2H])c(-c4ccc5c(ccc6ccccc65)c4)c([2H])c3[2H])c([2H])c2[2H])c([2H])c1[2H] ; [2H]c1c([2H])c(N(c2cccc(-c3ccc4ccccc4c3)c2)c2ccc3c(c2)C(c2ccccc2)(c2ccccc2)c2ccccc2-3)c([2H])c([2H])c1-c1ccc2c(ccc3ccccc32)c1]

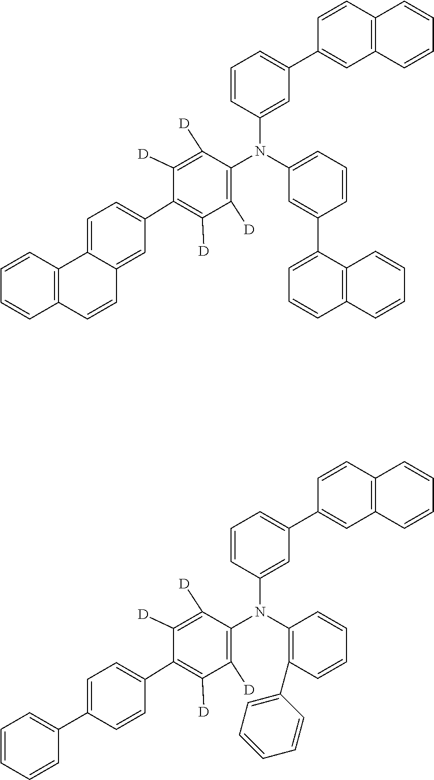 [SMILES: [2H]c1c([2H])c(N(c2cccc(-c3ccc4ccccc4c3)c2)c2cccc(-c3cccc4ccccc34)c2)c([2H])c([2H])c1-c1ccc2c(ccc3ccccc32)c1 ; [2H]c1c([2H])c(N(c2cccc(-c3ccc4ccccc4c3)c2)c2ccccc2-c2ccccc2)c([2H])c([2H])c1-c1ccc(-c2ccccc2)cc1]

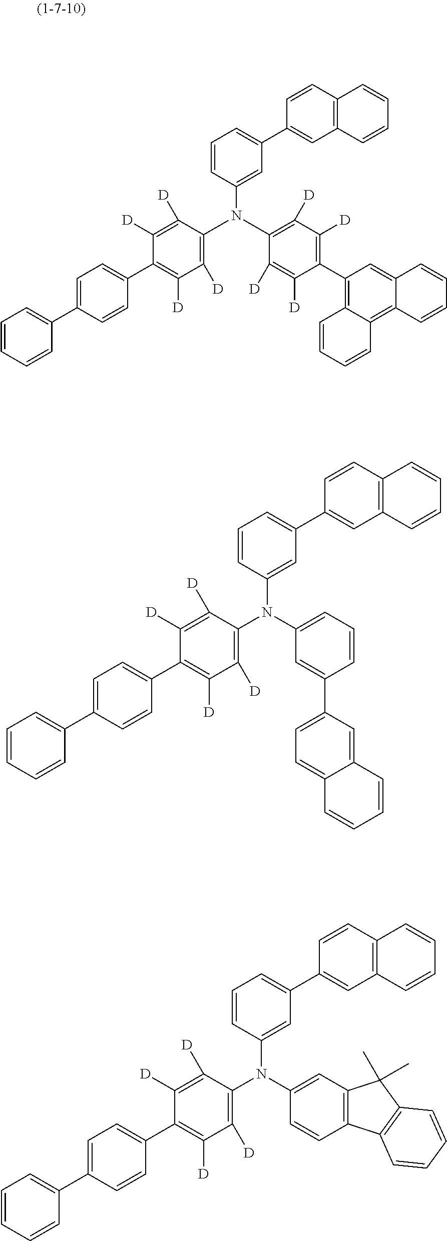 [SMILES: [2H]c1c([2H])c(N(c2cccc(-c3ccc4ccccc4c3)c2)c2c([2H])c([2H])c(-c3cc4ccccc4c4ccccc34)c([2H])c2[2H])c([2H])c([2H])c1-c1ccc(-c2ccccc2)cc1 ; [2H]c1c([2H])c(N(c2cccc(-c3ccc4ccccc4c3)c2)c2cccc(-c3ccc4ccccc4c3)c2)c([2H])c([2H])c1-c1ccc(-c2ccccc2)cc1 ; [2H]c1c([2H])c(N(c2cccc(-c3ccc4ccccc4c3)c2)c2ccc3c(c2)C(C)(C)c2ccccc2-3)c([2H])c([2H])c1-c1ccc(-c2ccccc2)cc1]

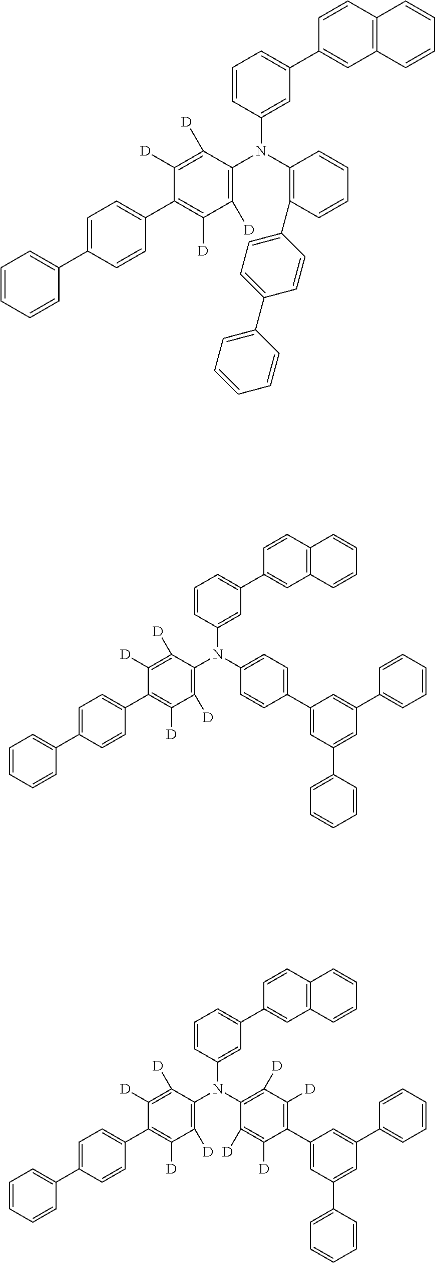 [SMILES: [2H]c1c([2H])c(N(c2cccc(-c3ccc4ccccc4c3)c2)c2ccccc2-c2ccc(-c3ccccc3)cc2)c([2H])c([2H])c1-c1ccc(-c2ccccc2)cc1 ; [2H]c1c([2H])c(N(c2ccc(-c3cc(-c4ccccc4)cc(-c4ccccc4)c3)cc2)c2cccc(-c3ccc4ccccc4c3)c2)c([2H])c([2H])c1-c1ccc(-c2ccccc2)cc1 ; [2H]c1c([2H])c(N(c2cccc(-c3ccc4ccccc4c3)c2)c2c([2H])c([2H])c(-c3cc(-c4ccccc4)cc(-c4ccccc4)c3)c([2H])c2[2H])c([2H])c([2H])c1-c1ccc(-c2ccccc2)cc1]

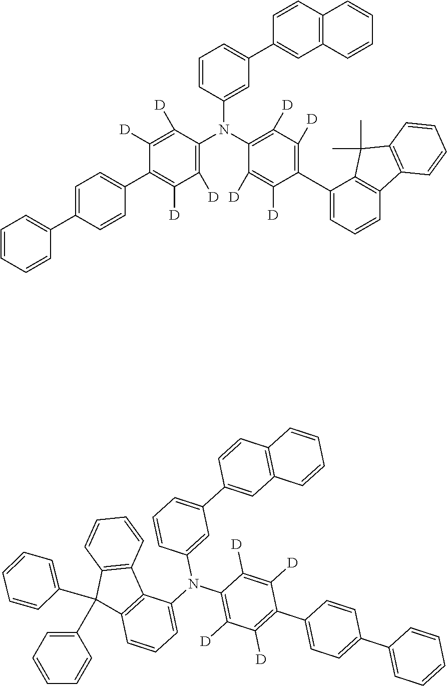 [SMILES: [2H]c1c([2H])c(N(c2cccc(-c3ccc4ccccc4c3)c2)c2c([2H])c([2H])c(-c3cccc4c3C(C)(C)c3ccccc3-4)c([2H])c2[2H])c([2H])c([2H])c1-c1ccc(-c2ccccc2)cc1 ; [2H]c1c([2H])c(N(c2cccc(-c3ccc4ccccc4c3)c2)c2cccc3c2-c2ccccc2C3(c2ccccc2)c2ccccc2)c([2H])c([2H])c1-c1ccc(-c2ccccc2)cc1]

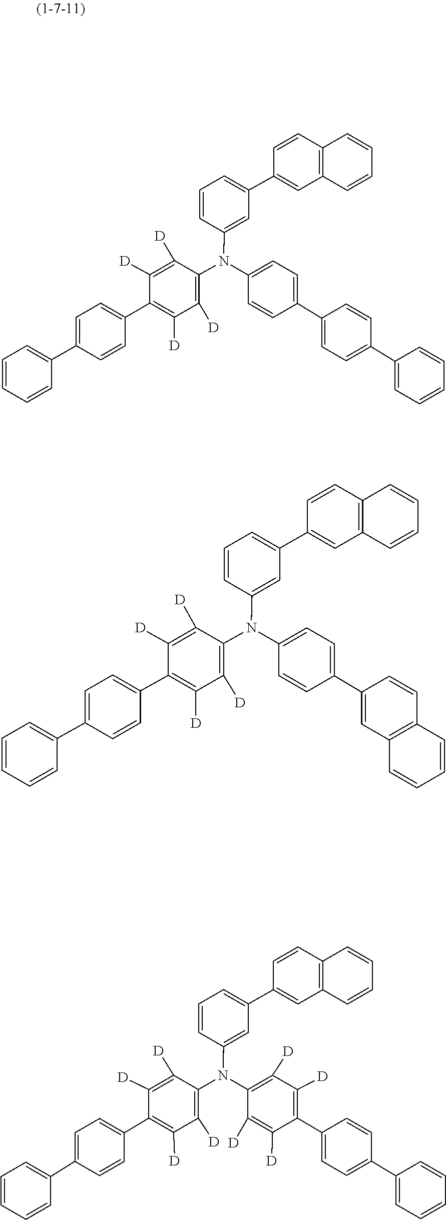 [SMILES: [2H]c1c([2H])c(N(c2ccc(-c3ccc(-c4ccccc4)cc3)cc2)c2cccc(-c3ccc4ccccc4c3)c2)c([2H])c([2H])c1-c1ccc(-c2ccccc2)cc1 ; [2H]c1c([2H])c(N(c2ccc(-c3ccc4ccccc4c3)cc2)c2cccc(-c3ccc4ccccc4c3)c2)c([2H])c([2H])c1-c1ccc(-c2ccccc2)cc1 ; [2H]c1c([2H])c(N(c2cccc(-c3ccc4ccccc4c3)c2)c2c([2H])c([2H])c(-c3ccc(-c4ccccc4)cc3)c([2H])c2[2H])c([2H])c([2H])c1-c1ccc(-c2ccccc2)cc1]

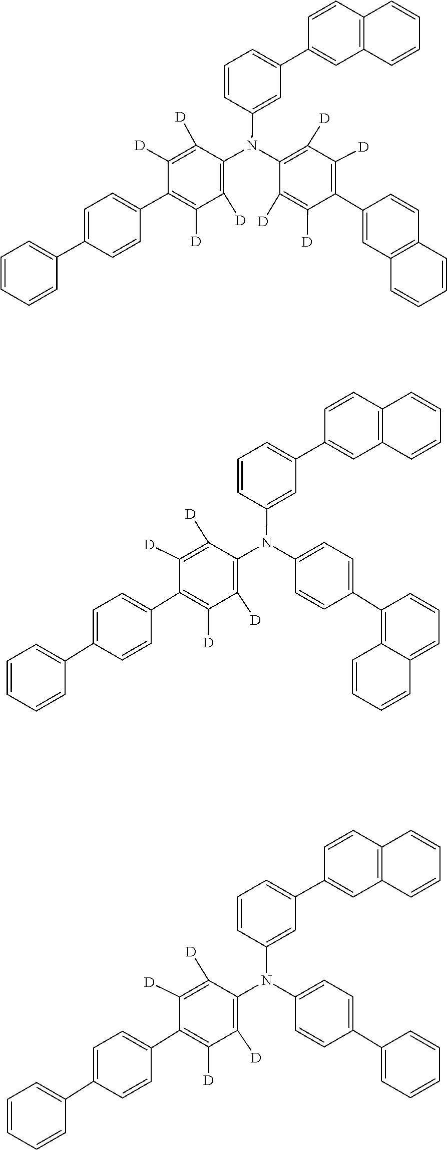 [SMILES: [2H]c1c([2H])c(N(c2cccc(-c3ccc4ccccc4c3)c2)c2c([2H])c([2H])c(-c3ccc4ccccc4c3)c([2H])c2[2H])c([2H])c([2H])c1-c1ccc(-c2ccccc2)cc1 ; [2H]c1c([2H])c(N(c2ccc(-c3cccc4ccccc34)cc2)c2cccc(-c3ccc4ccccc4c3)c2)c([2H])c([2H])c1-c1ccc(-c2ccccc2)cc1 ; [2H]c1c([2H])c(N(c2ccc(-c3ccccc3)cc2)c2cccc(-c3ccc4ccccc4c3)c2)c([2H])c([2H])c1-c1ccc(-c2ccccc2)cc1]

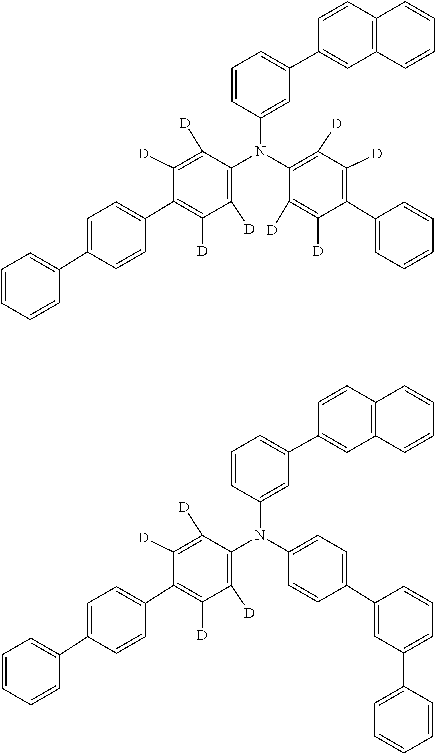 [SMILES: [2H]c1c([2H])c(N(c2cccc(-c3ccc4ccccc4c3)c2)c2c([2H])c([2H])c(-c3ccc(-c4ccccc4)cc3)c([2H])c2[2H])c([2H])c([2H])c1-c1ccccc1 ; [2H]c1c([2H])c(N(c2ccc(-c3cccc(-c4ccccc4)c3)cc2)c2cccc(-c3ccc4ccccc4c3)c2)c([2H])c([2H])c1-c1ccc(-c2ccccc2)cc1]

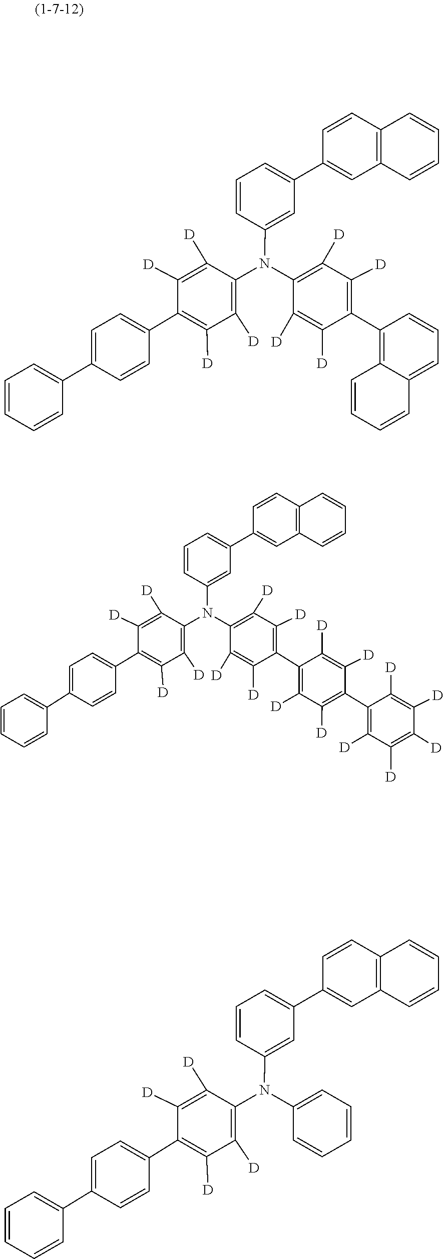 [SMILES: [2H]c1c([2H])c(N(c2cccc(-c3ccc4ccccc4c3)c2)c2c([2H])c([2H])c(-c3cccc4ccccc34)c([2H])c2[2H])c([2H])c([2H])c1-c1ccc(-c2ccccc2)cc1 ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5ccc(-c6ccccc6)cc5)c([2H])c4[2H])c([2H])c3[2H])c([2H])c2[2H])c([2H])c1[2H] ; [2H]c1c([2H])c(N(c2ccccc2)c2cccc(-c3ccc4ccccc4c3)c2)c([2H])c([2H])c1-c1ccc(-c2ccccc2)cc1]

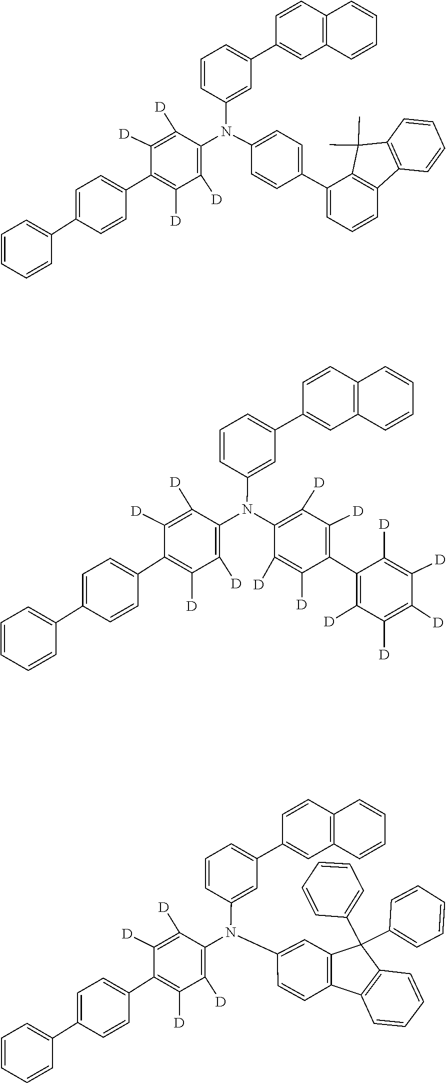 [SMILES: [2H]c1c([2H])c(N(c2ccc(-c3cccc4c3C(C)(C)c3ccccc3-4)cc2)c2cccc(-c3ccc4ccccc4c3)c2)c([2H])c([2H])c1-c1ccc(-c2ccccc2)cc1 ; [2H]c1c([2H])c([2H])c(-c2c([2H])c([2H])c(N(c3cccc(-c4ccc5ccccc5c4)c3)c3c([2H])c([2H])c(-c4ccc(-c5ccccc5)cc4)c([2H])c3[2H])c([2H])c2[2H])c([2H])c1[2H] ; [2H]c1c([2H])c(N(c2cccc(-c3ccc4ccccc4c3)c2)c2ccc3c(c2)C(c2ccccc2)(c2ccccc2)c2ccccc2-3)c([2H])c([2H])c1-c1ccc(-c2ccccc2)cc1]

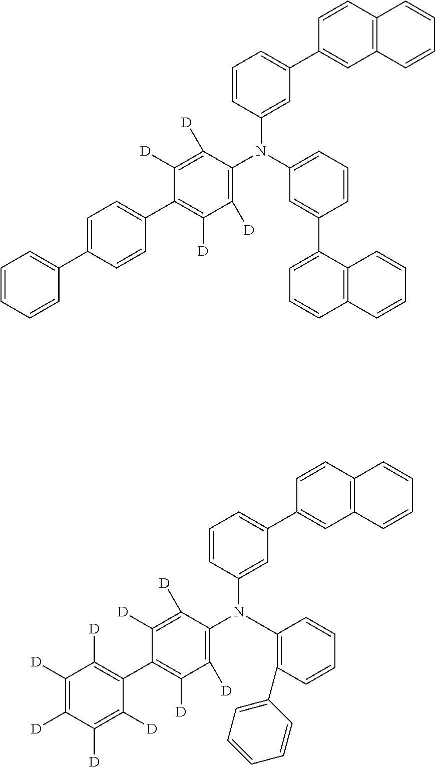 [SMILES: [2H]c1c([2H])c(N(c2cccc(-c3ccc4ccccc4c3)c2)c2cccc(-c3cccc4ccccc34)c2)c([2H])c([2H])c1-c1ccc(-c2ccccc2)cc1 ; [2H]c1c([2H])c([2H])c(-c2c([2H])c([2H])c(N(c3cccc(-c4ccc5ccccc5c4)c3)c3ccccc3-c3ccccc3)c([2H])c2[2H])c([2H])c1[2H]]

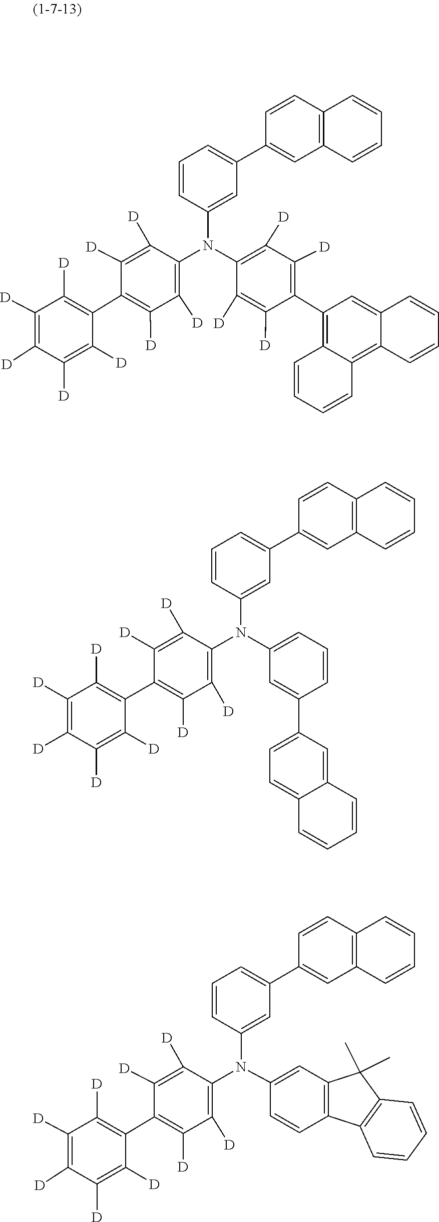 [SMILES: [2H]c1c([2H])c([2H])c(-c2c([2H])c([2H])c(N(c3cccc(-c4ccc5ccccc5c4)c3)c3c([2H])c([2H])c(-c4cc5ccccc5c5ccccc45)c([2H])c3[2H])c([2H])c2[2H])c([2H])c1[2H] ; [2H]c1c([2H])c([2H])c(-c2c([2H])c([2H])c(N(c3cccc(-c4ccc5ccccc5c4)c3)c3cccc(-c4ccc5ccccc5c4)c3)c([2H])c2[2H])c([2H])c1[2H] ; [2H]c1c([2H])c([2H])c(-c2c([2H])c([2H])c(N(c3cccc(-c4ccc5ccccc5c4)c3)c3ccc4c(c3)C(C)(C)c3ccccc3-4)c([2H])c2[2H])c([2H])c1[2H]]

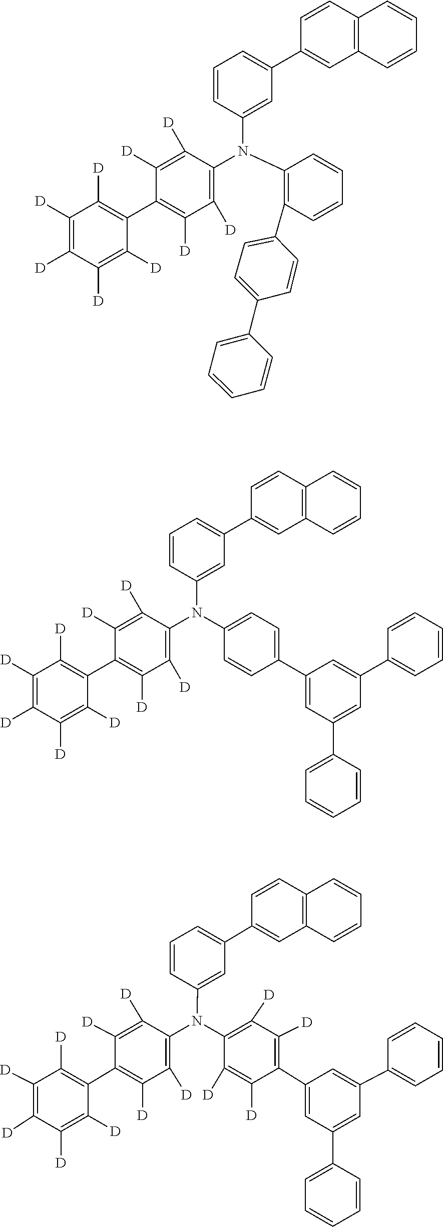 [SMILES: [2H]c1c([2H])c([2H])c(-c2c([2H])c([2H])c(N(c3cccc(-c4ccc5ccccc5c4)c3)c3ccccc3-c3ccc(-c4ccccc4)cc3)c([2H])c2[2H])c([2H])c1[2H] ; [2H]c1c([2H])c([2H])c(-c2c([2H])c([2H])c(N(c3ccc(-c4cc(-c5ccccc5)cc(-c5ccccc5)c4)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3cccc(-c4ccc5ccccc5c4)c3)c3c([2H])c([2H])c(-c4cc(-c5ccccc5)cc(-c5ccccc5)c4)c([2H])c3[2H])c([2H])c2[2H])c([2H])c1[2H]]

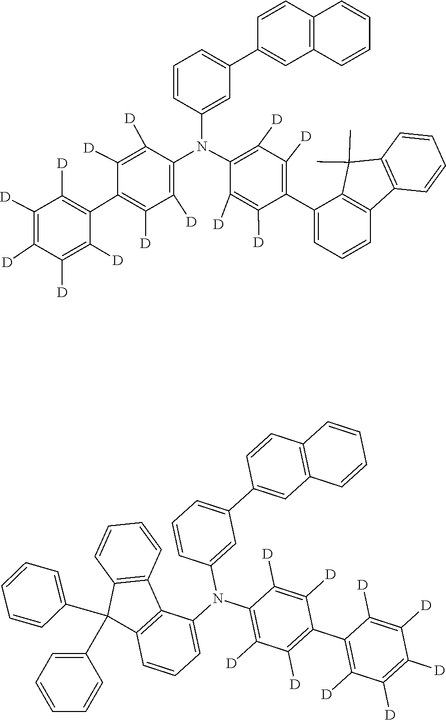 [SMILES: [2H]c1c([2H])c([2H])c(-c2c([2H])c([2H])c(N(c3cccc(-c4ccc5ccccc5c4)c3)c3c([2H])c([2H])c(-c4cccc5c4C(C)(C)c4ccccc4-5)c([2H])c3[2H])c([2H])c2[2H])c([2H])c1[2H] ; [2H]c1c([2H])c([2H])c(-c2c([2H])c([2H])c(N(c3cccc(-c4ccc5ccccc5c4)c3)c3cccc4c3-c3ccccc3C4(c3ccccc3)c3ccccc3)c([2H])c2[2H])c([2H])c1[2H]]

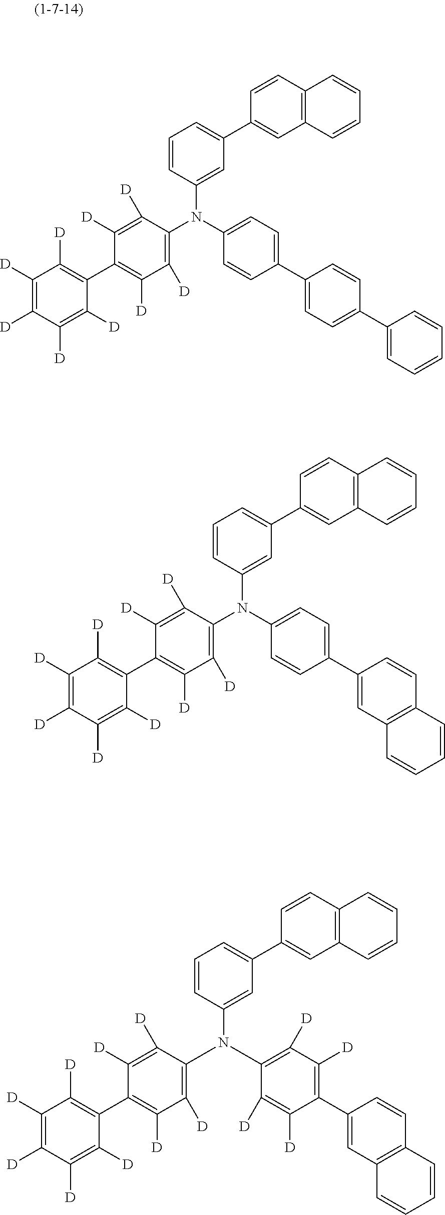 [SMILES: [2H]c1c([2H])c([2H])c(-c2c([2H])c([2H])c(N(c3ccc(-c4ccc(-c5ccccc5)cc4)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3ccc(-c4ccc5ccccc5c4)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3cccc(-c4ccc5ccccc5c4)c3)c3c([2H])c([2H])c(-c4ccc5ccccc5c4)c([2H])c3[2H])c([2H])c2[2H])c([2H])c1[2H]]

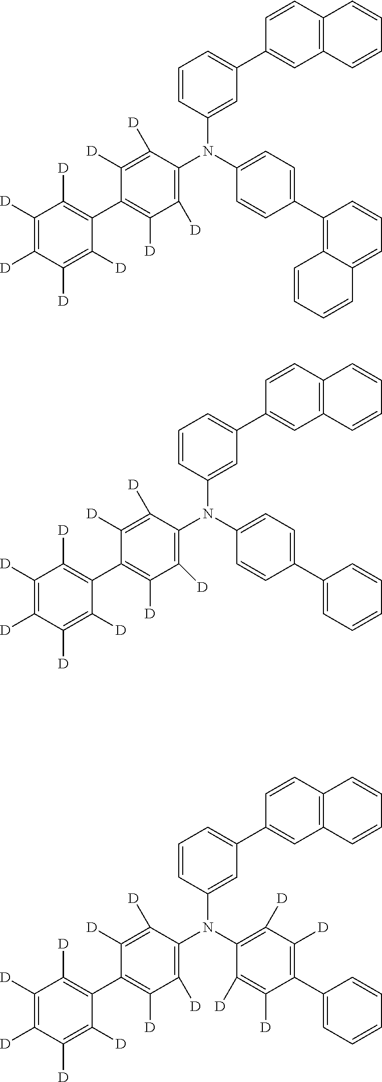 [SMILES: [2H]c1c([2H])c([2H])c(-c2c([2H])c([2H])c(N(c3ccc(-c4cccc5ccccc45)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3ccc(-c4ccccc4)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3cccc(-c4ccc5ccccc5c4)c3)c3c([2H])c([2H])c(-c4ccccc4)c([2H])c3[2H])c([2H])c2[2H])c([2H])c1[2H]]

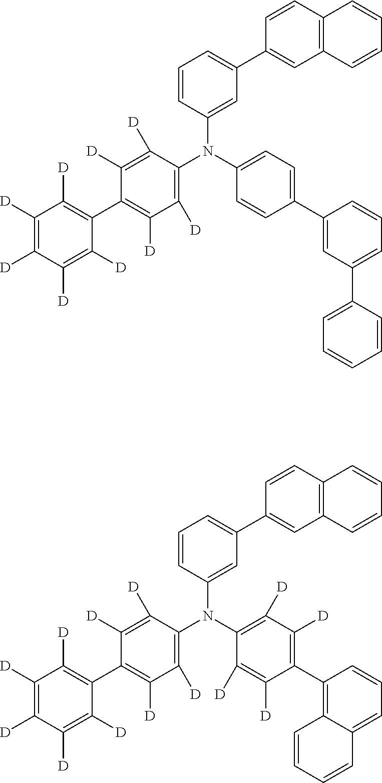 [SMILES: [2H]c1c([2H])c([2H])c(-c2c([2H])c([2H])c(N(c3ccc(-c4cccc(-c5ccccc5)c4)cc3)c3cccc(-c4ccc5ccccc5c4)c3)c([2H])c2[2H])c([2H])c1[2H] ; [2H]c1c([2H])c([2H])c(-c2c([2H])c([2H])c(N(c3cccc(-c4ccc5ccccc5c4)c3)c3c([2H])c([2H])c(-c4cccc5ccccc45)c([2H])c3[2H])c([2H])c2[2H])c([2H])c1[2H]]

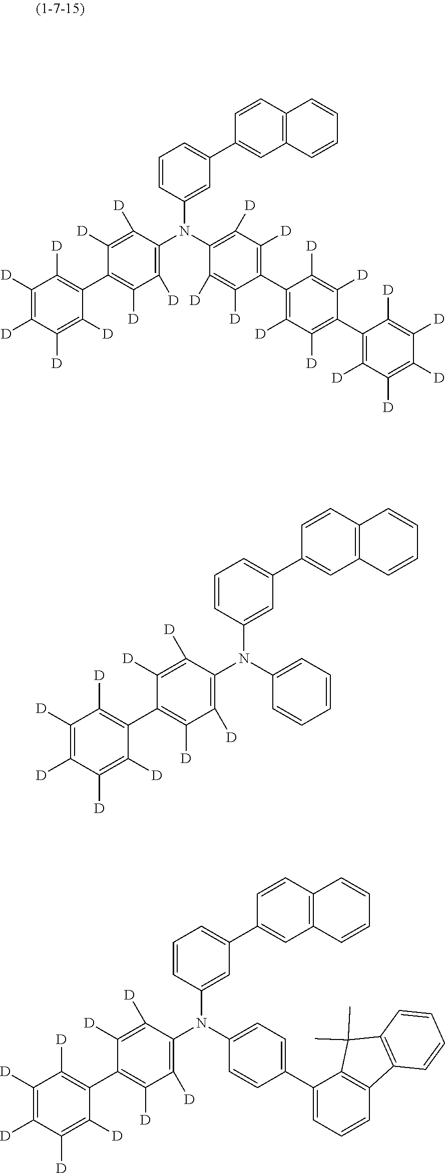 [SMILES: [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5c([2H])c([2H])c([2H])c([2H])c5[2H])c([2H])c4[2H])c([2H])c3[2H])c([2H])c2[2H])c([2H])c1[2H] ; [2H]c1c([2H])c([2H])c(-c2c([2H])c([2H])c(N(c3ccccc3)c3cccc(-c4ccc5ccccc5c4)c3)c([2H])c2[2H])c([2H])c1[2H] ; [2H]c1c([2H])c([2H])c(-c2c([2H])c([2H])c(N(c3ccc(-c4cccc5c4C(C)(C)c4ccccc4-5)cc3)c3cccc(-c4ccc5ccccc5c4)c3)c([2H])c2[2H])c([2H])c1[2H]]

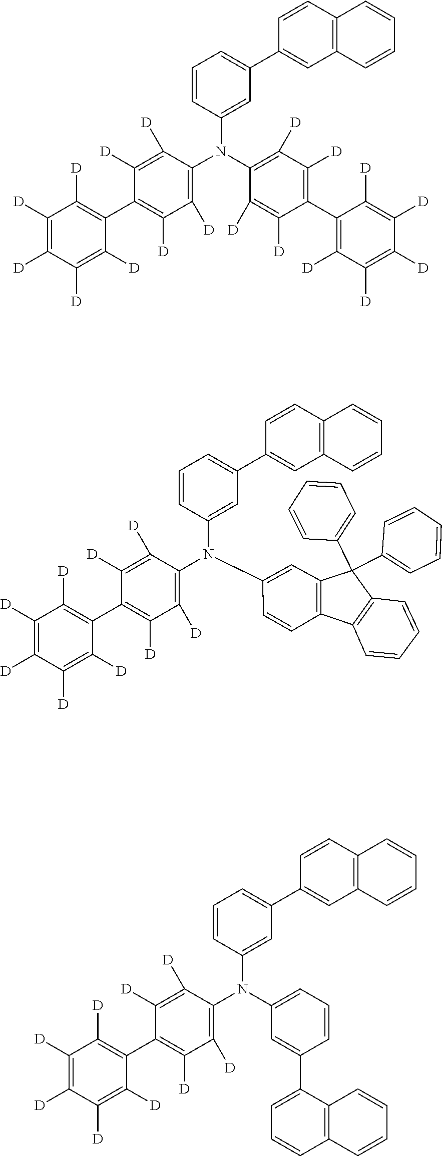 [SMILES: [2H]c1c([2H])c([2H])c(-c2c([2H])c([2H])c(N(c3cccc(-c4ccc5ccccc5c4)c3)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c(-c2c([2H])c([2H])c(N(c3cccc(-c4ccc5ccccc5c4)c3)c3ccc4c(c3)C(c3ccccc3)(c3ccccc3)c3ccccc3-4)c([2H])c2[2H])c([2H])c1[2H] ; [2H]c1c([2H])c([2H])c(-c2c([2H])c([2H])c(N(c3cccc(-c4ccc5ccccc5c4)c3)c3cccc(-c4cccc5ccccc45)c3)c([2H])c2[2H])c([2H])c1[2H]]

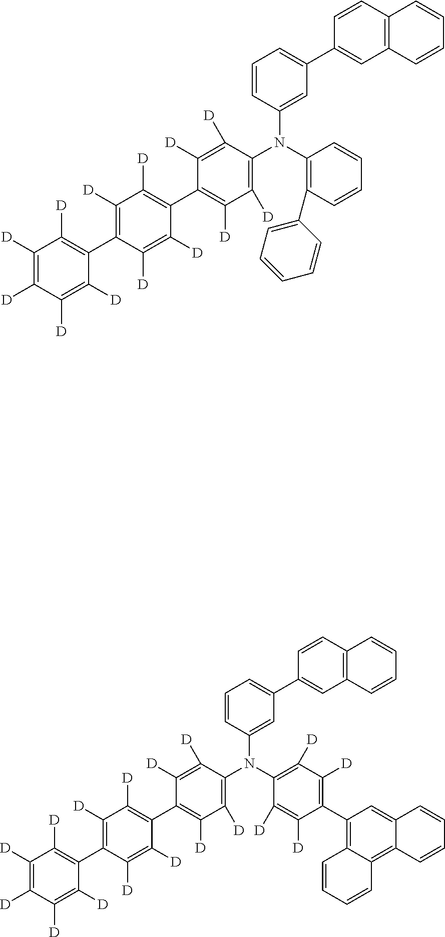 [SMILES: [2H]c1c([2H])c([2H])c(-c2c([2H])c([2H])c(-c3c([2H])c([2H])c(N(c4cccc(-c5ccc6ccccc6c5)c4)c4ccccc4-c4ccccc4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5cc6ccccc6c6ccccc56)c([2H])c4[2H])c([2H])c3[2H])c([2H])c2[2H])c([2H])c1[2H]]

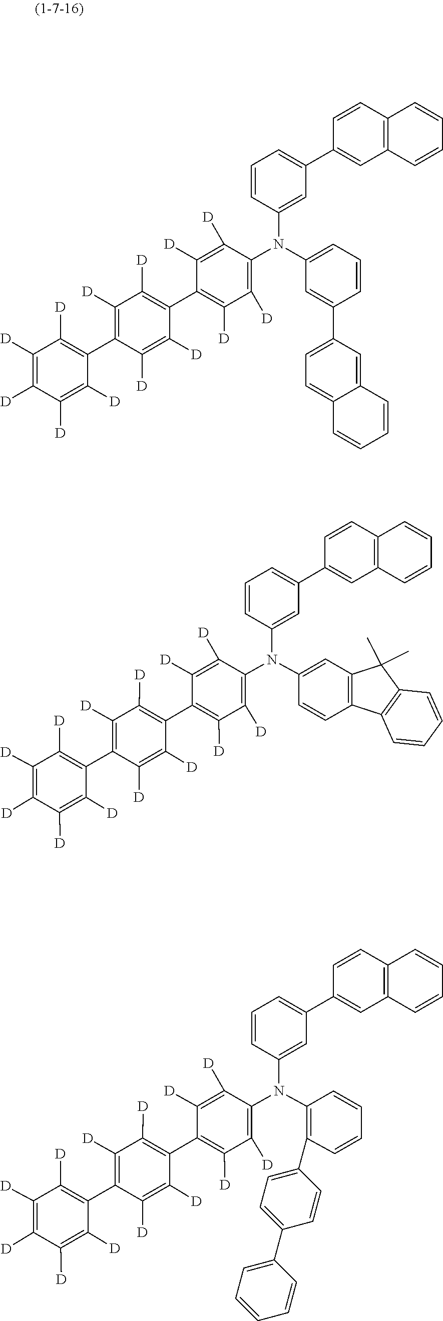 [SMILES: [2H]c1c([2H])c([2H])c(-c2c([2H])c([2H])c(-c3c([2H])c([2H])c(N(c4cccc(-c5ccc6ccccc6c5)c4)c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4ccc5c(c4)C(C)(C)c4ccccc4-5)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4ccccc4-c4ccc(-c5ccccc5)cc4)c([2H])c3[2H])c([2H])c2[2H])c([2H])c1[2H]]

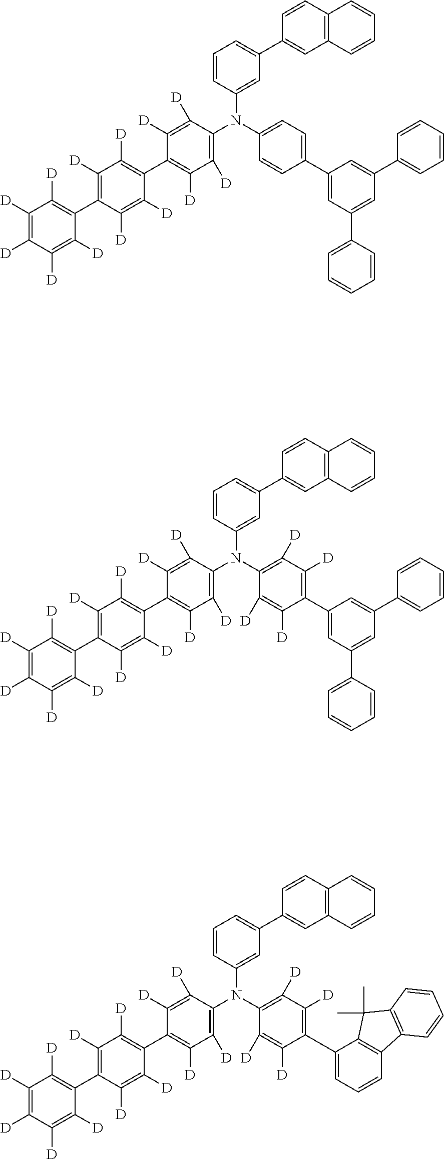

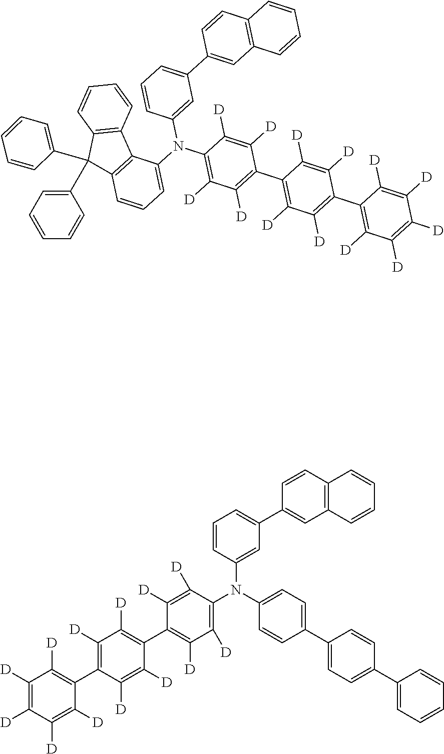 [SMILES: [2H]c1c([2H])c([2H])c(-c2c([2H])c([2H])c(-c3c([2H])c([2H])c(N(c4cccc(-c5ccc6ccccc6c5)c4)c4cccc5c4-c4ccccc4C5(c4ccccc4)c4ccccc4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5ccc(-c6ccccc6)cc5)cc4)c4cccc(-c5ccc6ccccc6c5)c4)c([2H])c3[2H])c([2H])c2[2H])c([2H])c1[2H]]

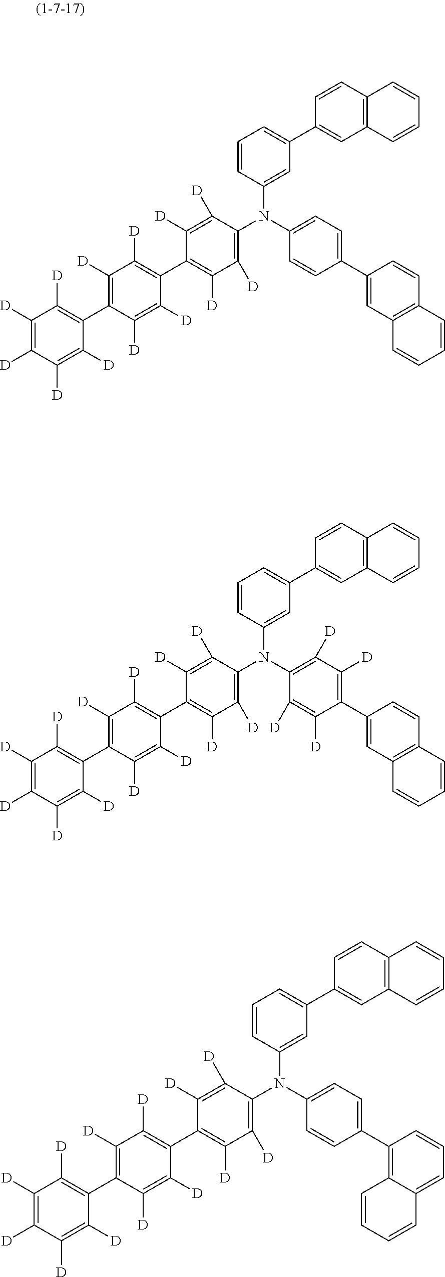 [SMILES: [2H]c1c([2H])c([2H])c(-c2c([2H])c([2H])c(-c3c([2H])c([2H])c(N(c4ccc(-c5ccc6ccccc6c5)cc4)c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5ccc6ccccc6c5)c([2H])c4[2H])c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc6ccccc56)cc4)c4cccc(-c5ccc6ccccc6c5)c4)c([2H])c3[2H])c([2H])c2[2H])c([2H])c1[2H]]

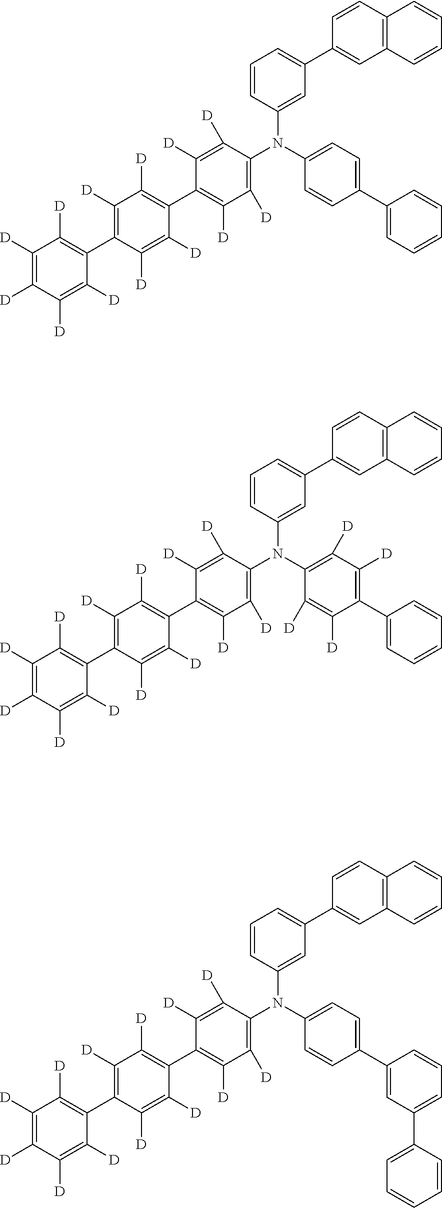 [SMILES: [2H]c1c([2H])c([2H])c(-c2c([2H])c([2H])c(-c3c([2H])c([2H])c(N(c4ccc(-c5ccccc5)cc4)c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5ccccc5)c([2H])c4[2H])c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccccc6)c5)cc4)c4cccc(-c5ccc6ccccc6c5)c4)c([2H])c3[2H])c([2H])c2[2H])c([2H])c1[2H]]

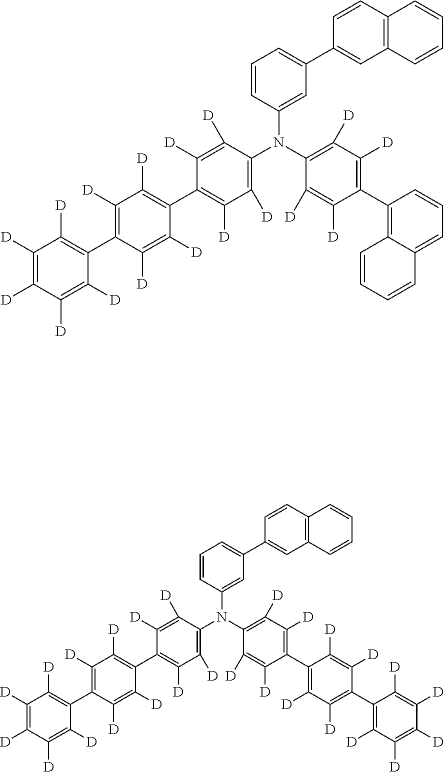 [SMILES: [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5cccc6ccccc56)c([2H])c4[2H])c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4c([2H])c([2H])c(-c5c([2H])c([2H])c(-c6c([2H])c([2H])c([2H])c([2H])c6[2H])c([2H])c5[2H])c([2H])c4[2H])c([2H])c3[2H])c([2H])c2[2H])c([2H])c1[2H]]

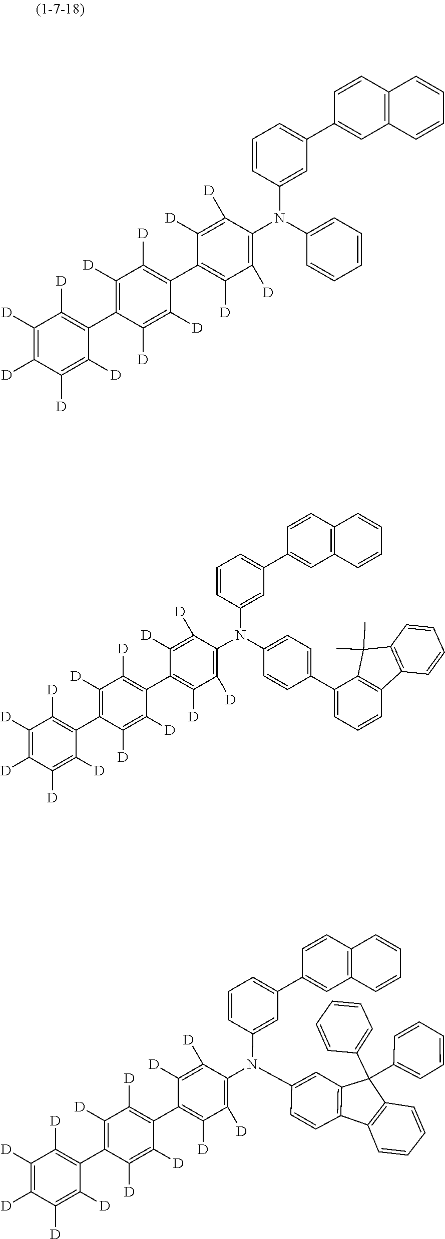 [SMILES: [2H]c1c([2H])c([2H])c(-c2c([2H])c([2H])c(-c3c([2H])c([2H])c(N(c4ccccc4)c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc6c5C(C)(C)c5ccccc5-6)cc4)c4cccc(-c5ccc6ccccc6c5)c4)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc(-c5ccc6ccccc6c5)c4)c4ccc5c(c4)C(c4ccccc4)(c4ccccc4)c4ccccc4-5)c([2H])c3[2H])c([2H])c2[2H])c([2H])c1[2H]]

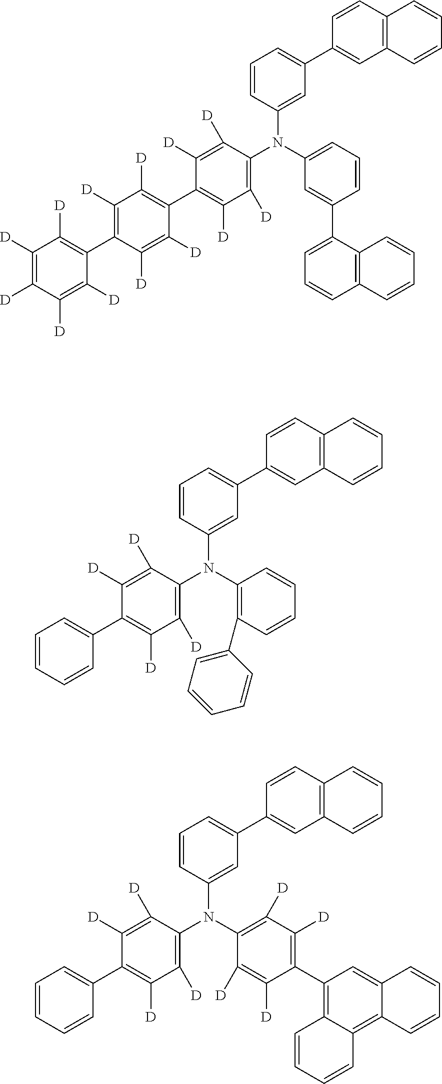 [SMILES: [2H]c1c([2H])c([2H])c(-c2c([2H])c([2H])c(-c3c([2H])c([2H])c(N(c4cccc(-c5ccc6ccccc6c5)c4)c4cccc(-c5cccc6ccccc56)c4)c([2H])c3[2H])c([2H])c2[2H])c([2H])c1[2H] ; [2H]c1c([2H])c(N(c2cccc(-c3ccc4ccccc4c3)c2)c2ccccc2-c2ccccc2)c([2H])c([2H])c1-c1ccccc1 ; [2H]c1c([2H])c(N(c2cccc(-c3ccc4ccccc4c3)c2)c2c([2H])c([2H])c(-c3cc4ccccc4c4ccccc34)c([2H])c2[2H])c([2H])c([2H])c1-c1ccccc1]

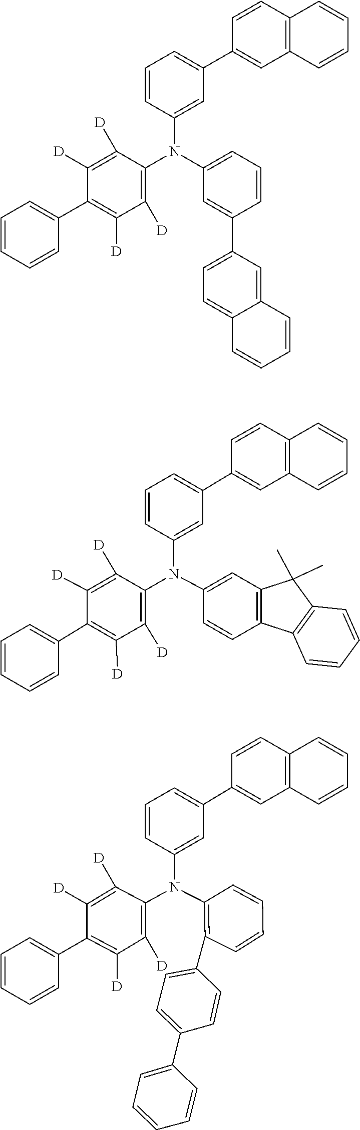

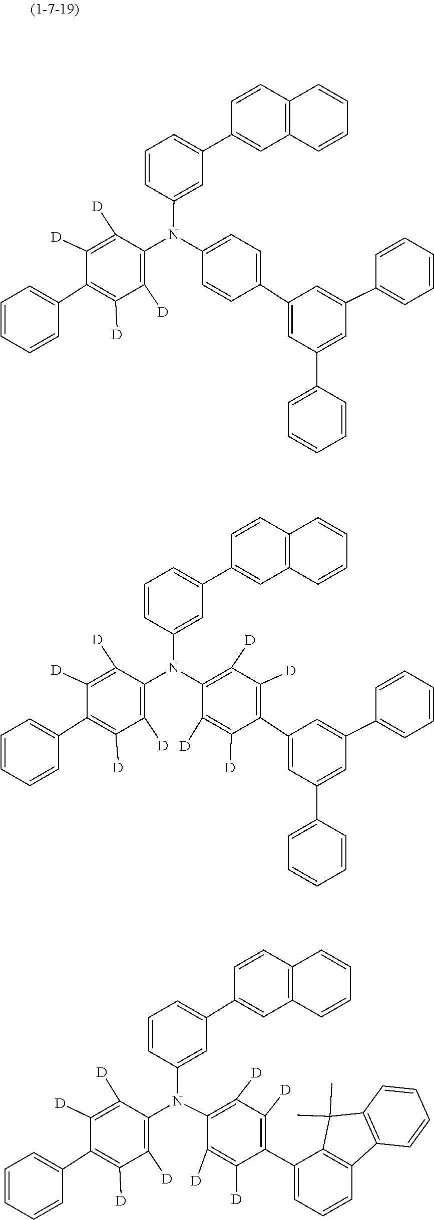 [SMILES: [2H]c1c([2H])c(N(c2ccc(-c3cc(-c4ccccc4)cc(-c4ccccc4)c3)cc2)c2cccc(-c3ccc4ccccc4c3)c2)c([2H])c([2H])c1-c1ccccc1 ; [2H]c1c([2H])c(N(c2cccc(-c3ccc4ccccc4c3)c2)c2c([2H])c([2H])c(-c3cc(-c4ccccc4)cc(-c4ccccc4)c3)c([2H])c2[2H])c([2H])c([2H])c1-c1ccccc1 ; [2H]c1c([2H])c(N(c2cccc(-c3ccc4ccccc4c3)c2)c2c([2H])c([2H])c(-c3cccc4c3C(C)(C)c3ccccc3-4)c([2H])c2[2H])c([2H])c([2H])c1-c1ccccc1]

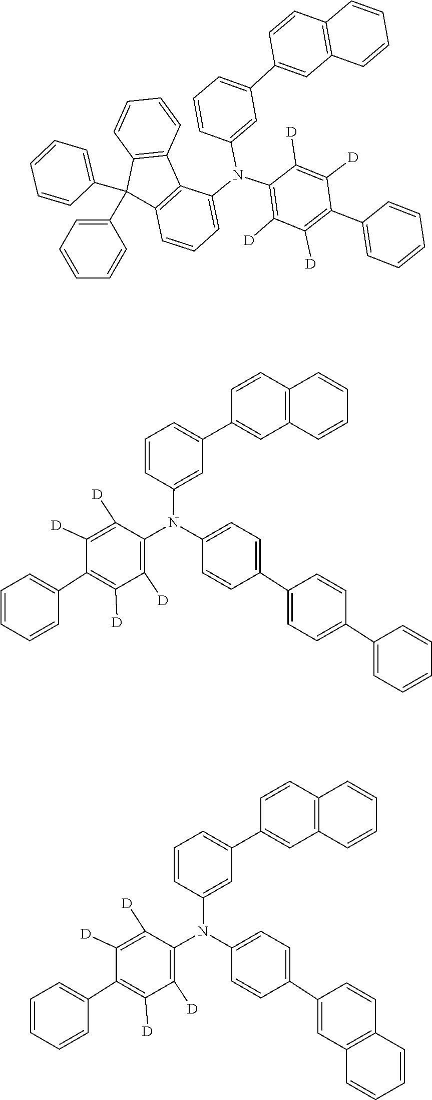 [SMILES: [2H]c1c([2H])c(N(c2cccc(-c3ccc4ccccc4c3)c2)c2cccc3c2-c2ccccc2C3(c2ccccc2)c2ccccc2)c([2H])c([2H])c1-c1ccccc1 ; [2H]c1c([2H])c(N(c2ccc(-c3ccc(-c4ccccc4)cc3)cc2)c2cccc(-c3ccc4ccccc4c3)c2)c([2H])c([2H])c1-c1ccccc1 ; [2H]c1c([2H])c(N(c2ccc(-c3ccc4ccccc4c3)cc2)c2cccc(-c3ccc4ccccc4c3)c2)c([2H])c([2H])c1-c1ccccc1]

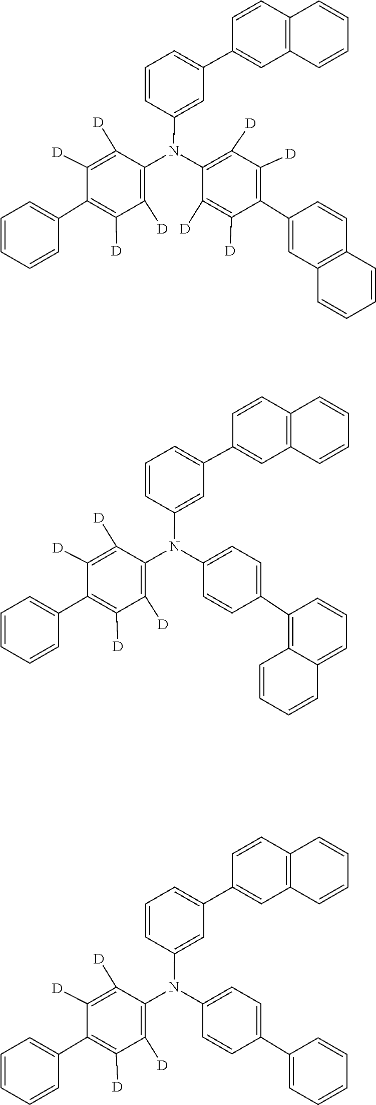 [SMILES: [2H]c1c([2H])c(N(c2cccc(-c3ccc4ccccc4c3)c2)c2c([2H])c([2H])c(-c3ccc4ccccc4c3)c([2H])c2[2H])c([2H])c([2H])c1-c1ccccc1 ; [2H]c1c([2H])c(N(c2ccc(-c3cccc4ccccc34)cc2)c2cccc(-c3ccc4ccccc4c3)c2)c([2H])c([2H])c1-c1ccccc1 ; [2H]c1c([2H])c(N(c2ccc(-c3ccccc3)cc2)c2cccc(-c3ccc4ccccc4c3)c2)c([2H])c([2H])c1-c1ccccc1]

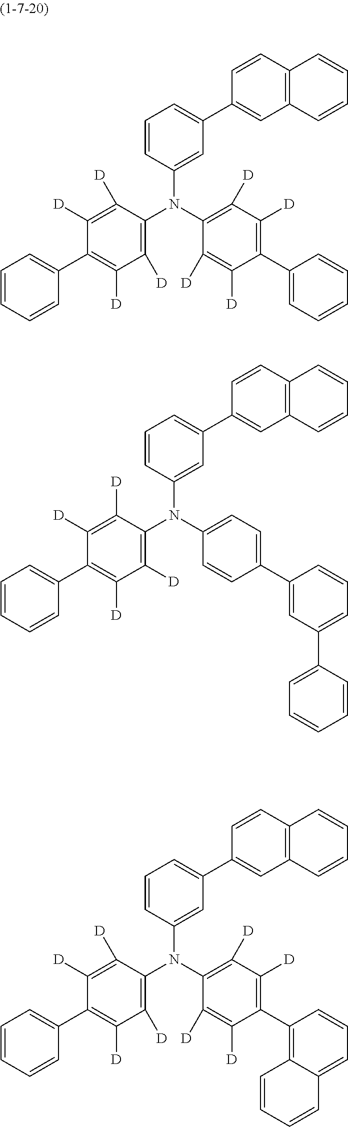

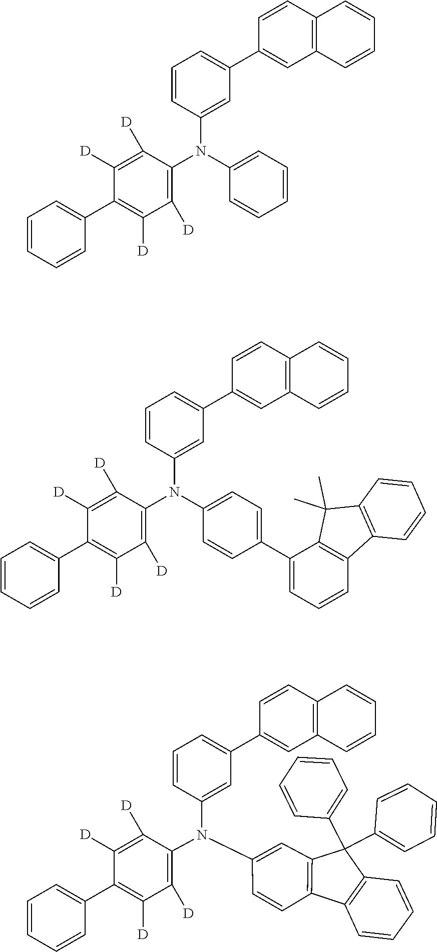 [SMILES: [2H]c1c([2H])c(N(c2ccccc2)c2cccc(-c3ccc4ccccc4c3)c2)c([2H])c([2H])c1-c1ccccc1 ; [2H]c1c([2H])c(N(c2ccc(-c3cccc4c3C(C)(C)c3ccccc3-4)cc2)c2cccc(-c3ccc4ccccc4c3)c2)c([2H])c([2H])c1-c1ccccc1 ; [2H]c1c([2H])c(N(c2cccc(-c3ccc4ccccc4c3)c2)c2ccc3c(c2)C(c2ccccc2)(c2ccccc2)c2ccccc2-3)c([2H])c([2H])c1-c1ccccc1]

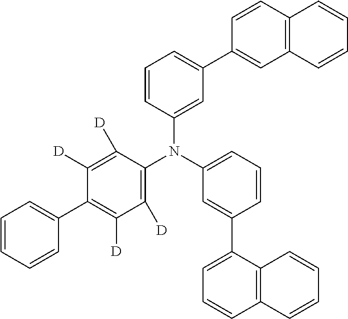 [SMILES: [2H]c1c([2H])c(N(c2cccc(-c3ccc4ccccc4c3)c2)c2cccc(-c3cccc4ccccc34)c2)c([2H])c([2H])c1-c1ccccc1]

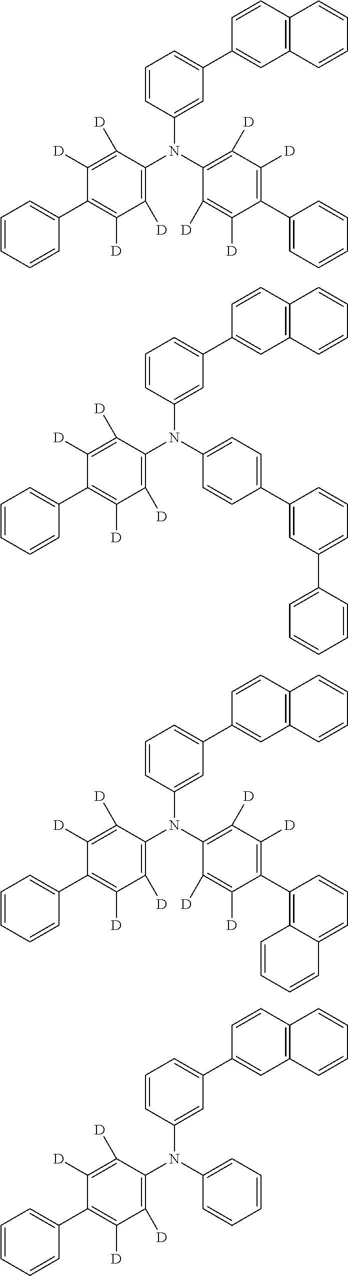

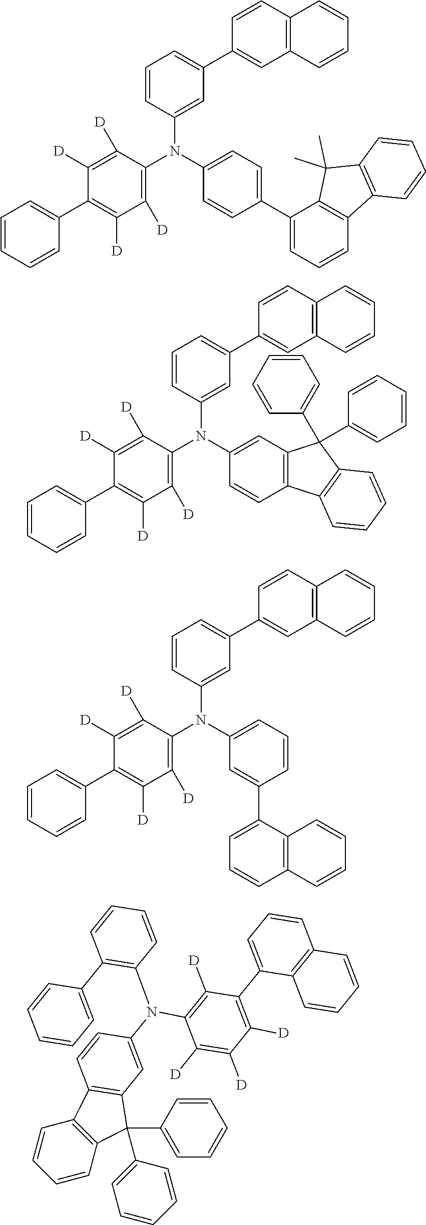

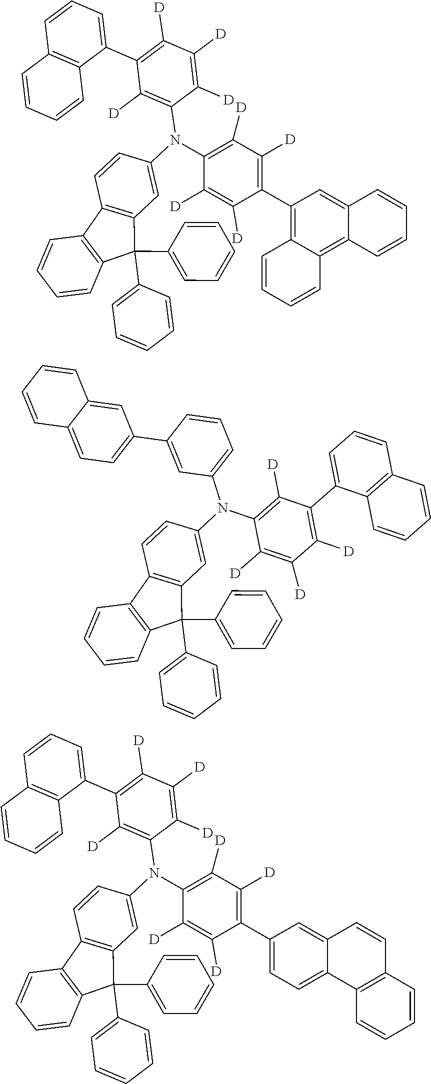

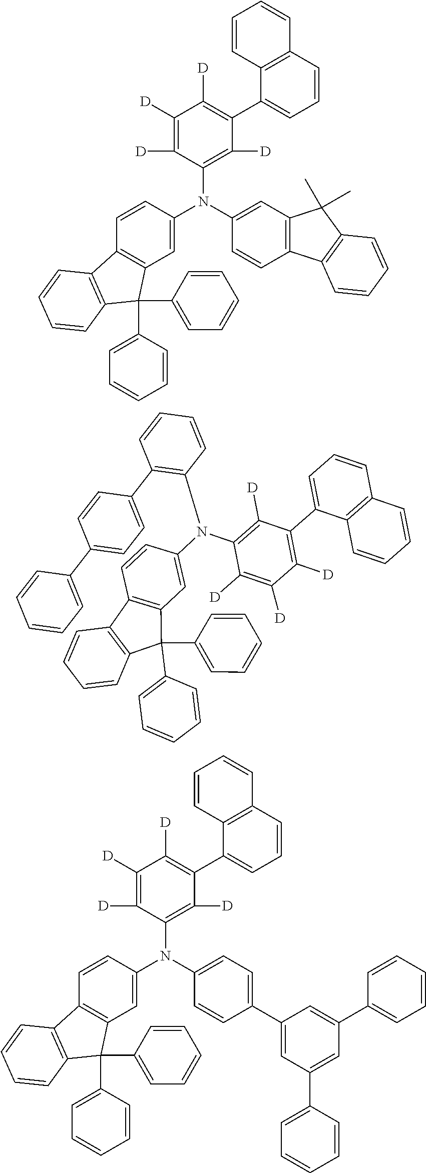

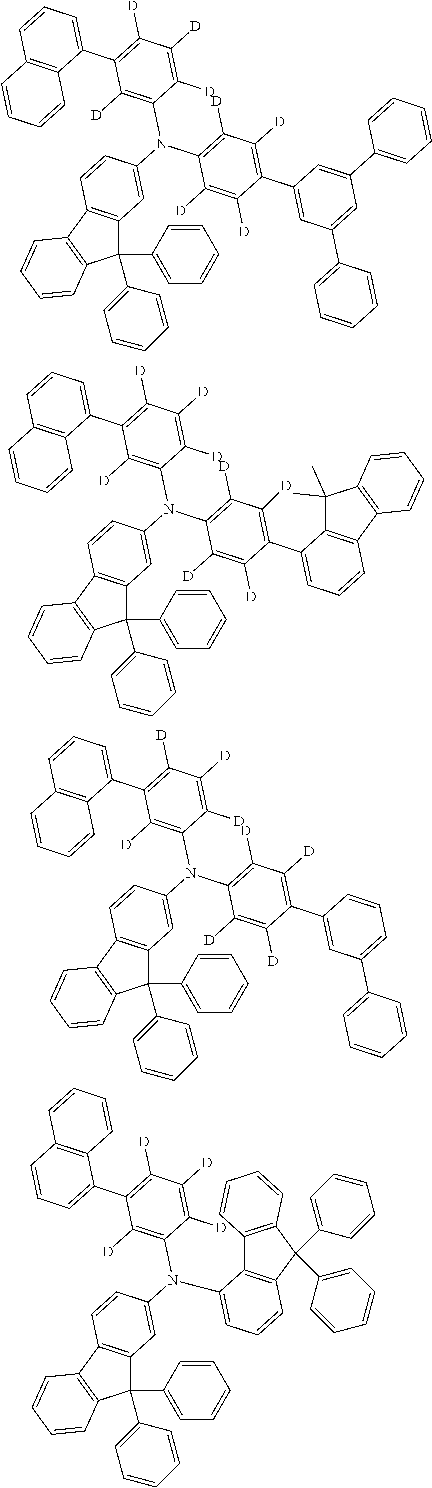

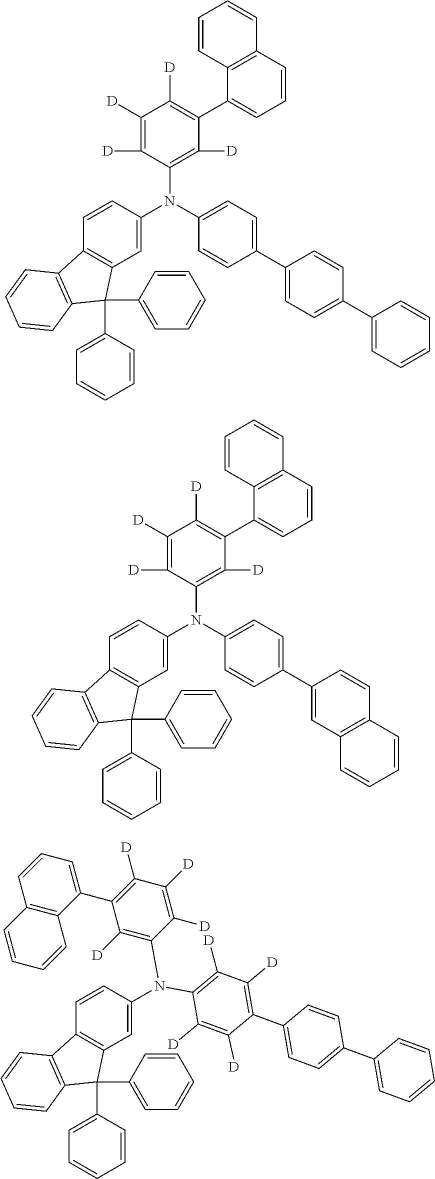

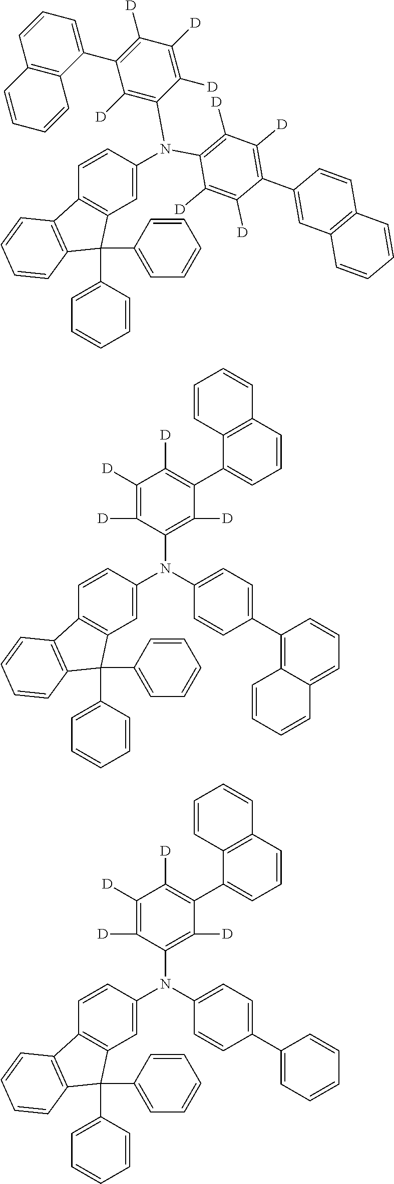 [SMILES: [2H]c1c([2H])c(-c2cccc3ccccc23)c([2H])c(N(c2ccc3c(c2)C(c2ccccc2)(c2ccccc2)c2ccccc2-3)c2c([2H])c([2H])c(-c3ccc4ccccc4c3)c([2H])c2[2H])c1[2H] ; [2H]c1c([2H])c(-c2cccc3ccccc23)c([2H])c(N(c2ccc(-c3cccc4ccccc34)cc2)c2ccc3c(c2)C(c2ccccc2)(c2ccccc2)c2ccccc2-3)c1[2H] ; [2H]c1c([2H])c(-c2cccc3ccccc23)c([2H])c(N(c2ccc(-c3ccccc3)cc2)c2ccc3c(c2)C(c2ccccc2)(c2ccccc2)c2ccccc2-3)c1[2H]]

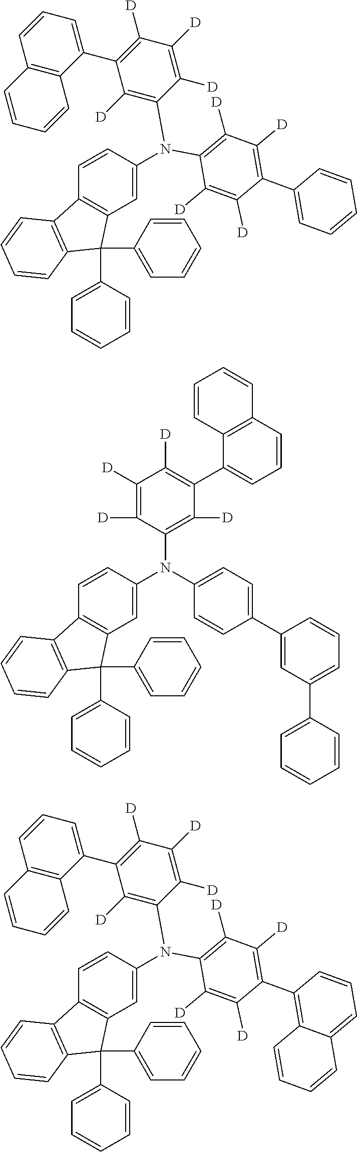 [SMILES: [2H]c1c([2H])c(-c2cccc3ccccc23)c([2H])c(N(c2ccc3c(c2)C(c2ccccc2)(c2ccccc2)c2ccccc2-3)c2c([2H])c([2H])c(-c3ccccc3)c([2H])c2[2H])c1[2H] ; [2H]c1c([2H])c(-c2cccc3ccccc23)c([2H])c(N(c2ccc(-c3cccc(-c4ccccc4)c3)cc2)c2ccc3c(c2)C(c2ccccc2)(c2ccccc2)c2ccccc2-3)c1[2H] ; [2H]c1c([2H])c(-c2cccc3ccccc23)c([2H])c(N(c2ccc3c(c2)C(c2ccccc2)(c2ccccc2)c2ccccc2-3)c2c([2H])c([2H])c(-c3cccc4ccccc34)c([2H])c2[2H])c1[2H]]

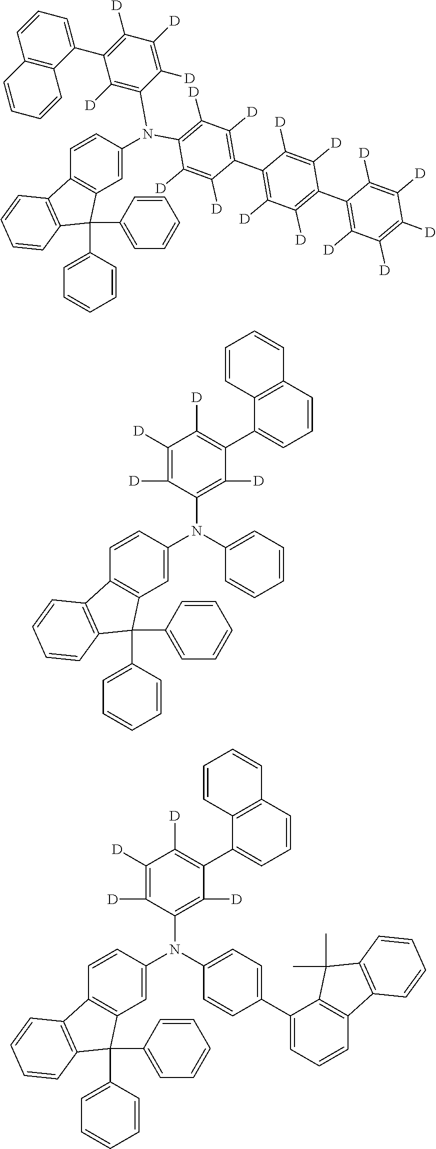 [SMILES: [2H]c1c([2H])c([2H])c(-c2c([2H])c([2H])c(-c3c([2H])c([2H])c(N(c4ccc5c(c4)C(c4ccccc4)(c4ccccc4)c4ccccc4-5)c4c([2H])c([2H])c([2H])c(-c5cccc6ccccc56)c4[2H])c([2H])c3[2H])c([2H])c2[2H])c([2H])c1[2H] ; [2H]c1c([2H])c(-c2cccc3ccccc23)c([2H])c(N(c2ccccc2)c2ccc3c(c2)C(c2ccccc2)(c2ccccc2)c2ccccc2-3)c1[2H] ; [2H]c1c([2H])c(-c2cccc3ccccc23)c([2H])c(N(c2ccc(-c3cccc4c3C(C)(C)c3ccccc3-4)cc2)c2ccc3c(c2)C(c2ccccc2)(c2ccccc2)c2ccccc2-3)c1[2H]]

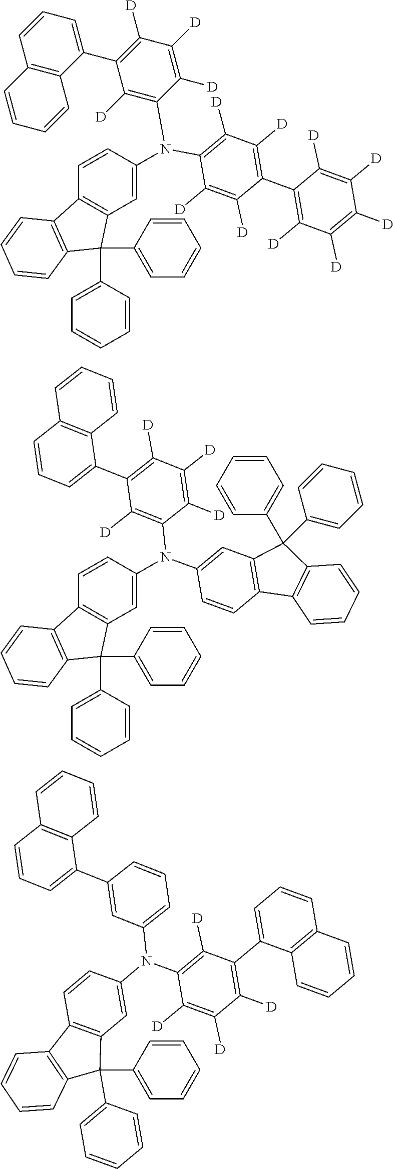

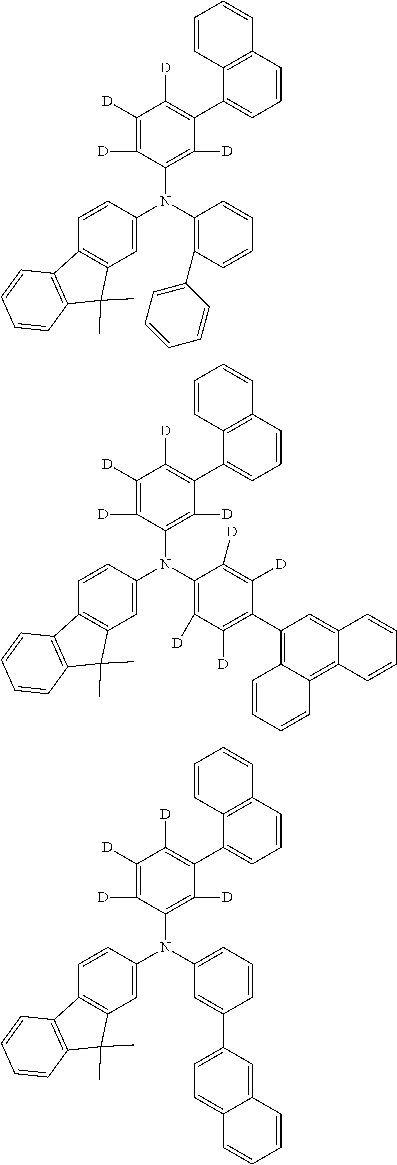

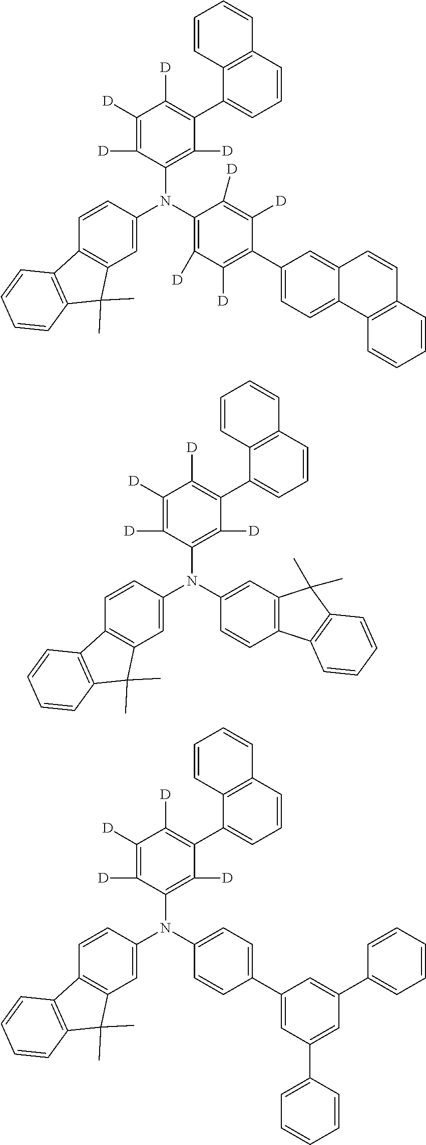 [SMILES: [2H]c1c([2H])c(-c2cccc3ccccc23)c([2H])c(N(c2ccc3c(c2)C(C)(C)c2ccccc2-3)c2c([2H])c([2H])c(-c3ccc4c(ccc5ccccc54)c3)c([2H])c2[2H])c1[2H] ; [2H]c1c([2H])c(-c2cccc3ccccc23)c([2H])c(N(c2ccc3c(c2)C(C)(C)c2ccccc2-3)c2ccc3c(c2)C(C)(C)c2ccccc2-3)c1[2H] ; [2H]c1c([2H])c(-c2cccc3ccccc23)c([2H])c(N(c2ccc(-c3cc(-c4ccccc4)cc(-c4ccccc4)c3)cc2)c2ccc3c(c2)C(C)(C)c2ccccc2-3)c1[2H]]

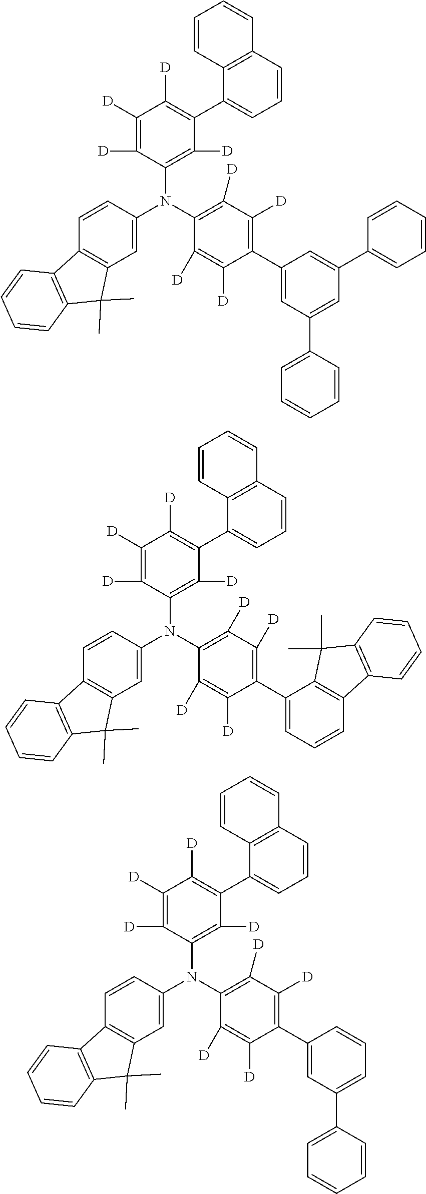 [SMILES: [2H]c1c([2H])c(-c2cccc3ccccc23)c([2H])c(N(c2ccc3c(c2)C(C)(C)c2ccccc2-3)c2c([2H])c([2H])c(-c3cc(-c4ccccc4)cc(-c4ccccc4)c3)c([2H])c2[2H])c1[2H] ; [2H]c1c([2H])c(-c2cccc3ccccc23)c([2H])c(N(c2ccc3c(c2)C(C)(C)c2ccccc2-3)c2c([2H])c([2H])c(-c3cccc4c3C(C)(C)c3ccccc3-4)c([2H])c2[2H])c1[2H] ; [2H]c1c([2H])c(-c2cccc3ccccc23)c([2H])c(N(c2ccc3c(c2)C(C)(C)c2ccccc2-3)c2c([2H])c([2H])c(-c3cccc(-c4ccccc4)c3)c([2H])c2[2H])c1[2H]]

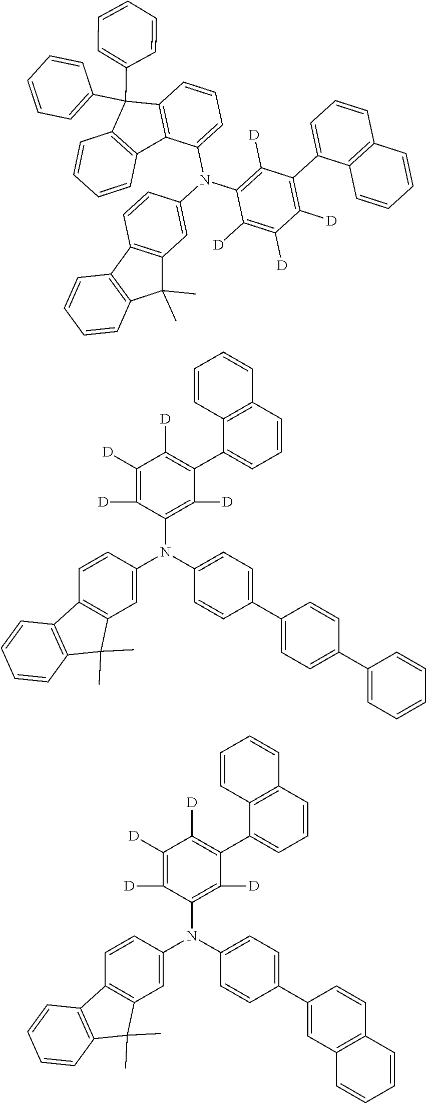 [SMILES: [2H]c1c([2H])c(-c2cccc3ccccc23)c([2H])c(N(c2ccc3c(c2)C(C)(C)c2ccccc2-3)c2cccc3c2-c2ccccc2C3(c2ccccc2)c2ccccc2)c1[2H] ; [2H]c1c([2H])c(-c2cccc3ccccc23)c([2H])c(N(c2ccc(-c3ccc(-c4ccccc4)cc3)cc2)c2ccc3c(c2)C(C)(C)c2ccccc2-3)c1[2H] ; [2H]c1c([2H])c(-c2cccc3ccccc23)c([2H])c(N(c2ccc(-c3ccc4ccccc4c3)cc2)c2ccc3c(c2)C(C)(C)c2ccccc2-3)c1[2H]]

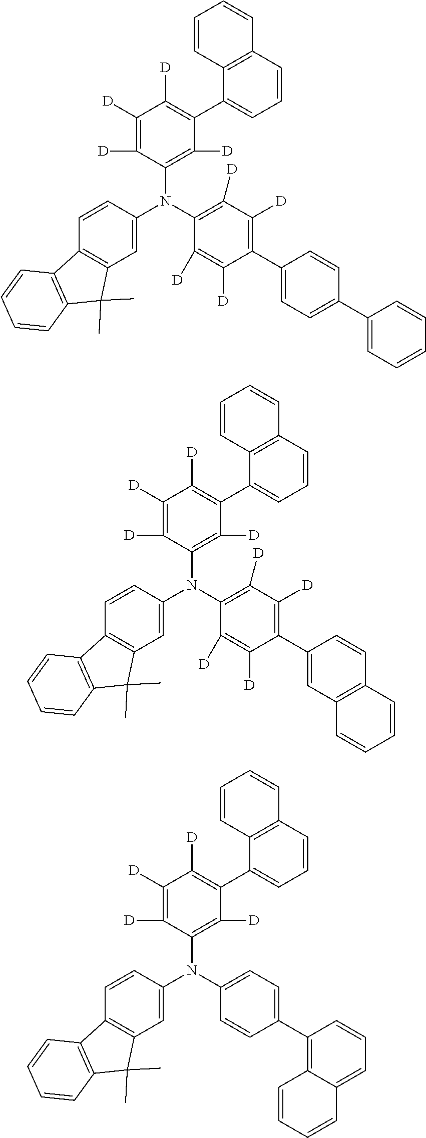 [SMILES: [2H]c1c([2H])c(-c2cccc3ccccc23)c([2H])c(N(c2ccc3c(c2)C(C)(C)c2ccccc2-3)c2c([2H])c([2H])c(-c3ccc(-c4ccccc4)cc3)c([2H])c2[2H])c1[2H] ; [2H]c1c([2H])c(-c2cccc3ccccc23)c([2H])c(N(c2ccc3c(c2)C(C)(C)c2ccccc2-3)c2c([2H])c([2H])c(-c3ccc4ccccc4c3)c([2H])c2[2H])c1[2H] ; [2H]c1c([2H])c(-c2cccc3ccccc23)c([2H])c(N(c2ccc(-c3cccc4ccccc34)cc2)c2ccc3c(c2)C(C)(C)c2ccccc2-3)c1[2H]]

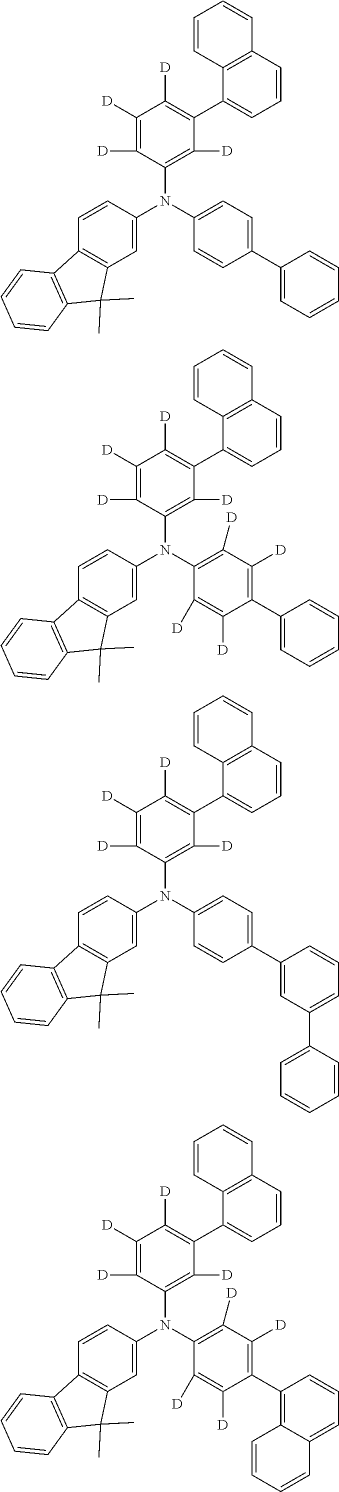

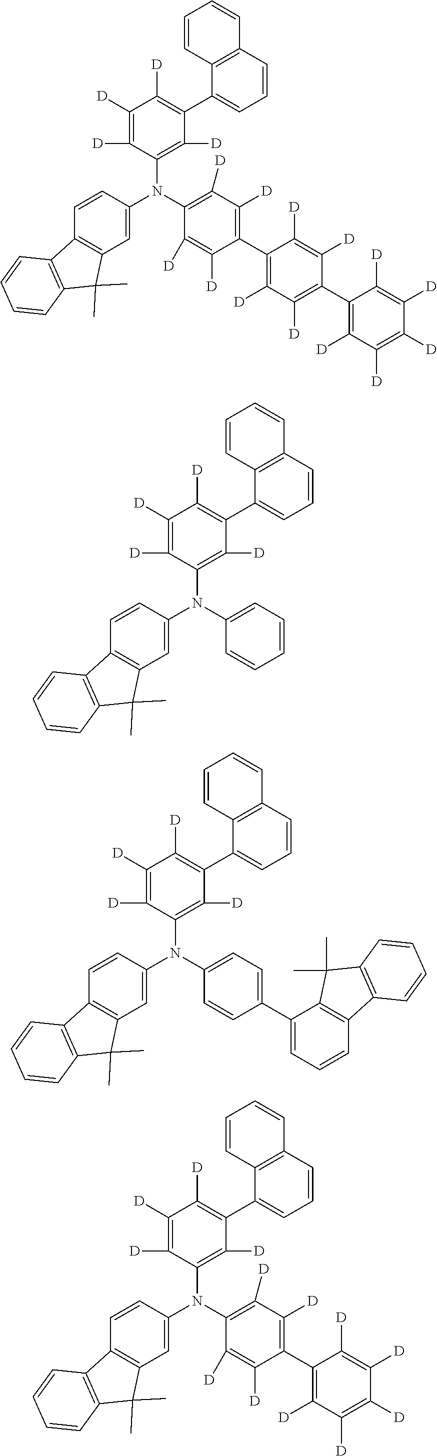

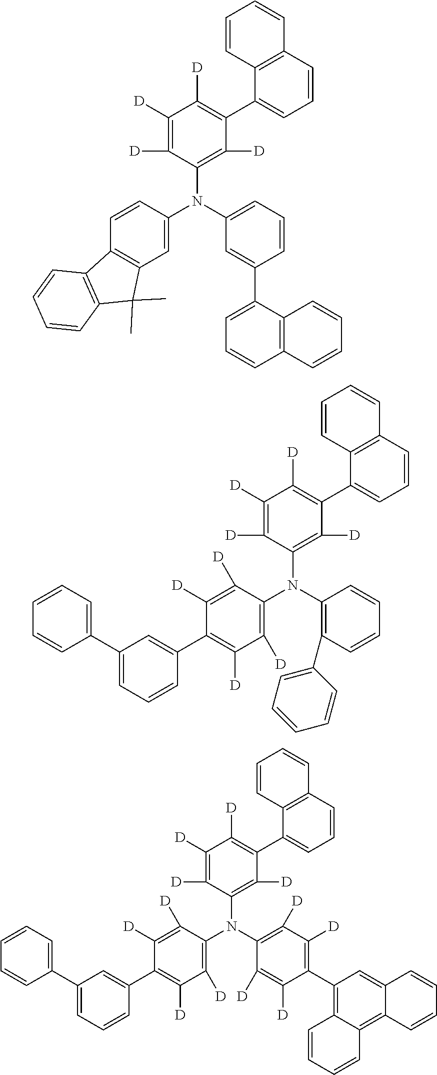

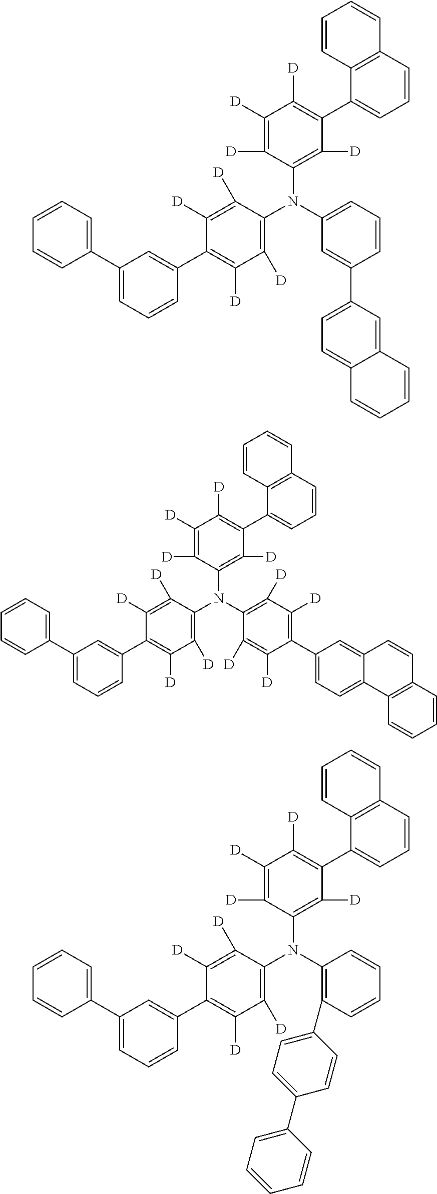

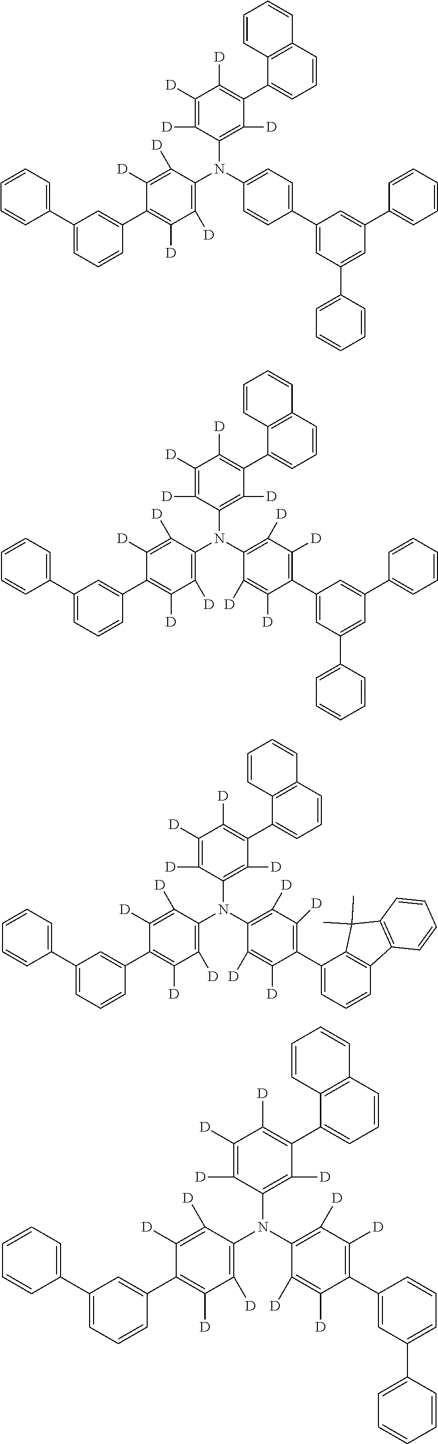

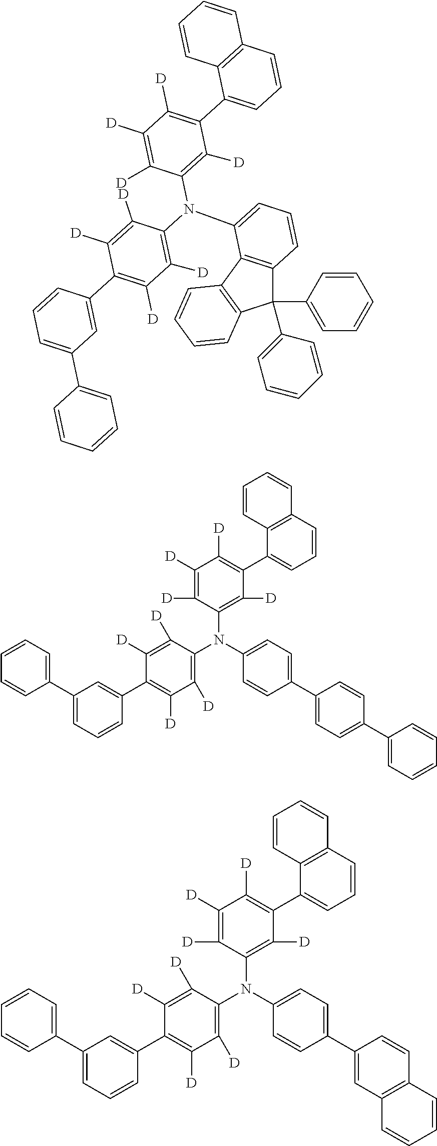 [SMILES: [2H]c1c([2H])c(-c2cccc3ccccc23)c([2H])c(N(c2cccc3c2-c2ccccc2C3(c2ccccc2)c2ccccc2)c2c([2H])c([2H])c(-c3cccc(-c4ccccc4)c3)c([2H])c2[2H])c1[2H] ; [2H]c1c([2H])c(-c2cccc3ccccc23)c([2H])c(N(c2ccc(-c3ccc(-c4ccccc4)cc3)cc2)c2c([2H])c([2H])c(-c3cccc(-c4ccccc4)c3)c([2H])c2[2H])c1[2H] ; [2H]c1c([2H])c(-c2cccc3ccccc23)c([2H])c(N(c2ccc(-c3ccc4ccccc4c3)cc2)c2c([2H])c([2H])c(-c3cccc(-c4ccccc4)c3)c([2H])c2[2H])c1[2H]]

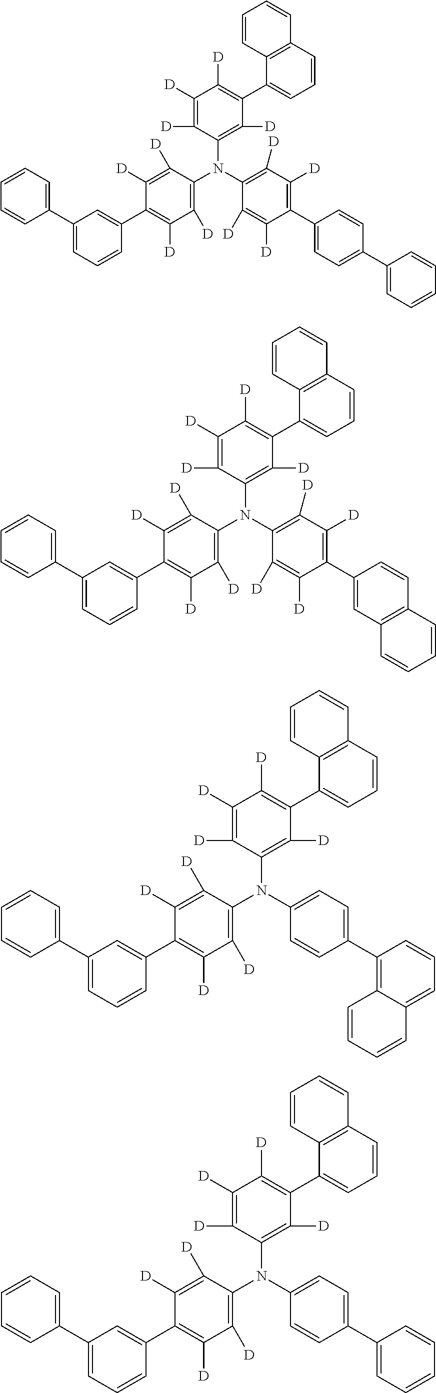

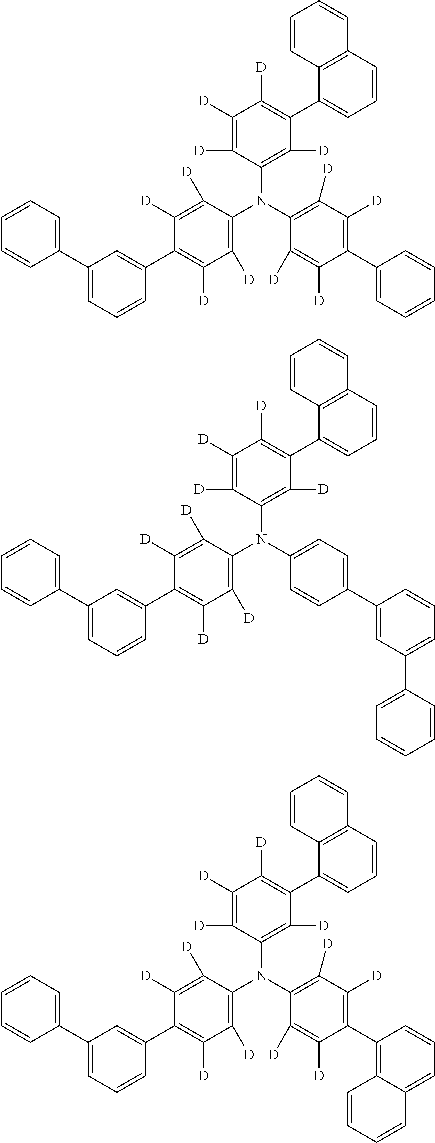

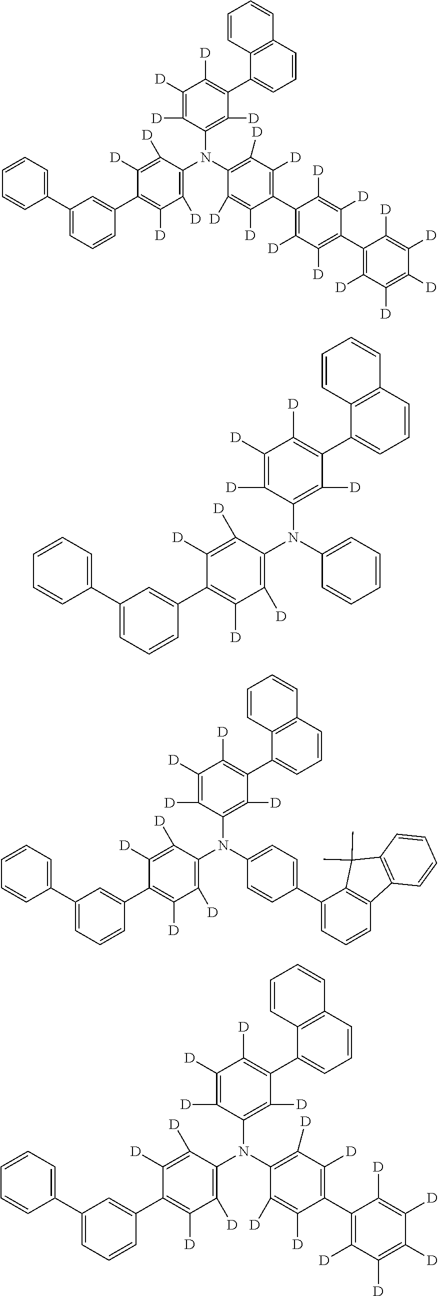

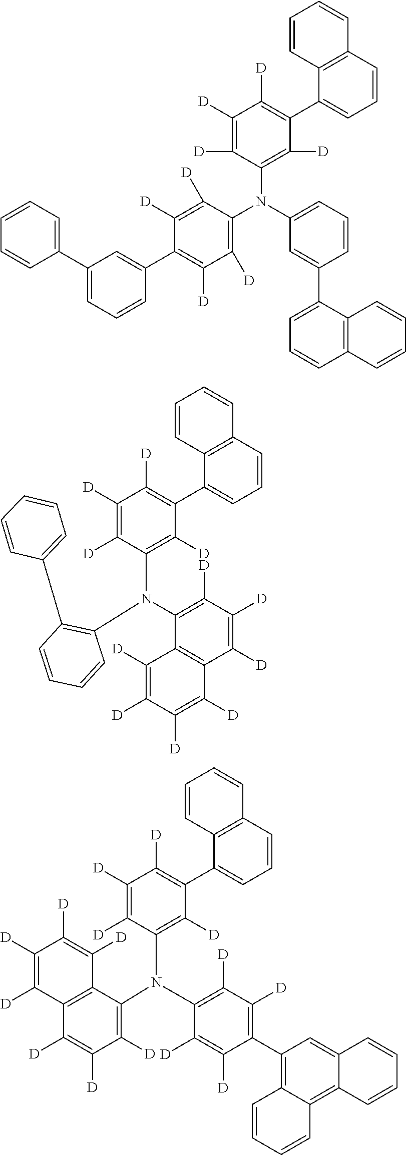

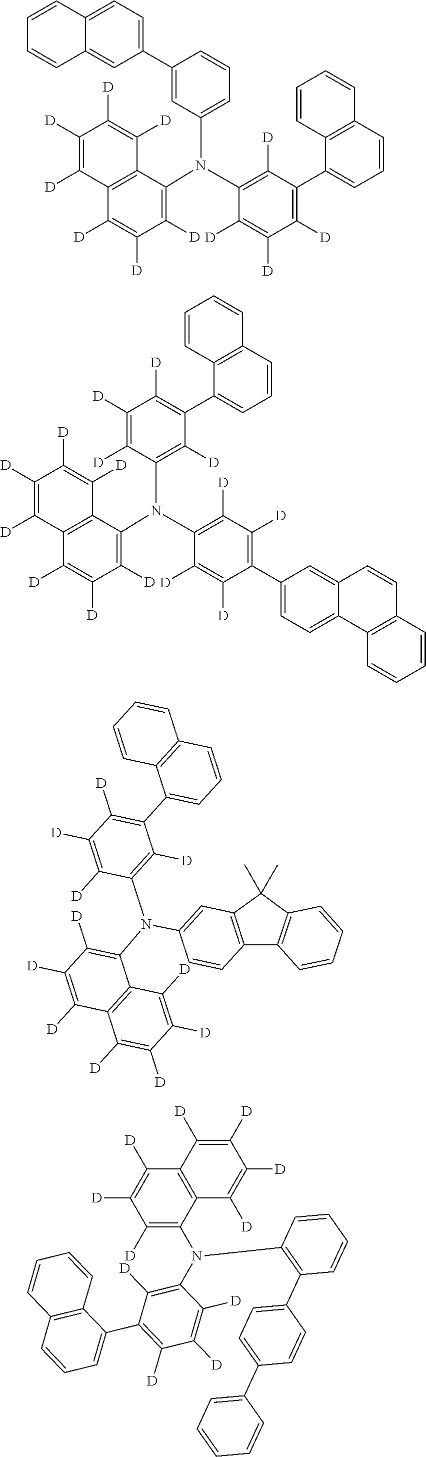

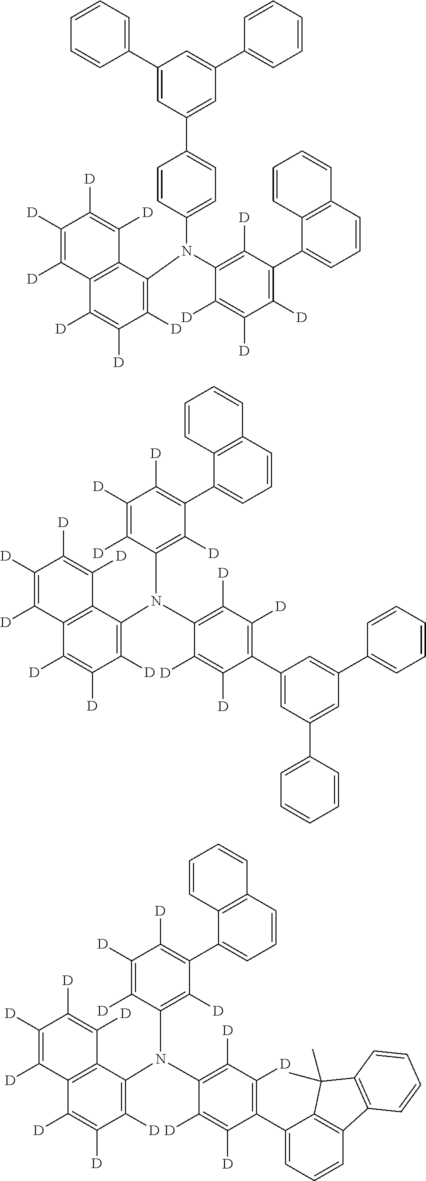 [SMILES: [2H]c1c([2H])c(-c2cccc3ccccc23)c([2H])c(N(c2ccc(-c3cc(-c4ccccc4)cc(-c4ccccc4)c3)cc2)c2c([2H])c([2H])c([2H])c3c([2H])c([2H])c([2H])c([2H])c23)c1[2H] ; [2H]c1c([2H])c(-c2cccc3ccccc23)c([2H])c(N(c2c([2H])c([2H])c(-c3cc(-c4ccccc4)cc(-c4ccccc4)c3)c([2H])c2[2H])c2c([2H])c([2H])c([2H])c3c([2H])c([2H])c([2H])c([2H])c23)c1[2H] ; [2H]c1c([2H])c(-c2cccc3ccccc23)c([2H])c(N(c2c([2H])c([2H])c(-c3cccc4c3C(C)(C)c3ccccc3-4)c([2H])c2[2H])c2c([2H])c([2H])c([2H])c3c([2H])c([2H])c([2H])c([2H])c23)c1[2H]]

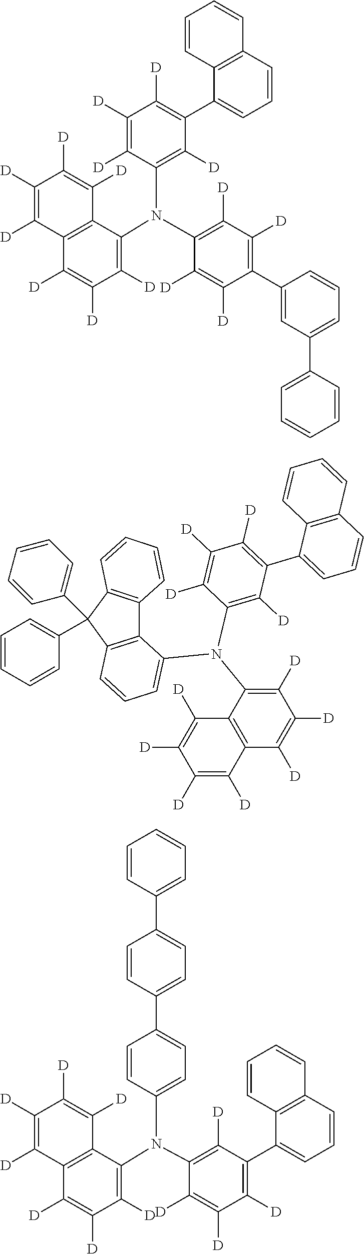

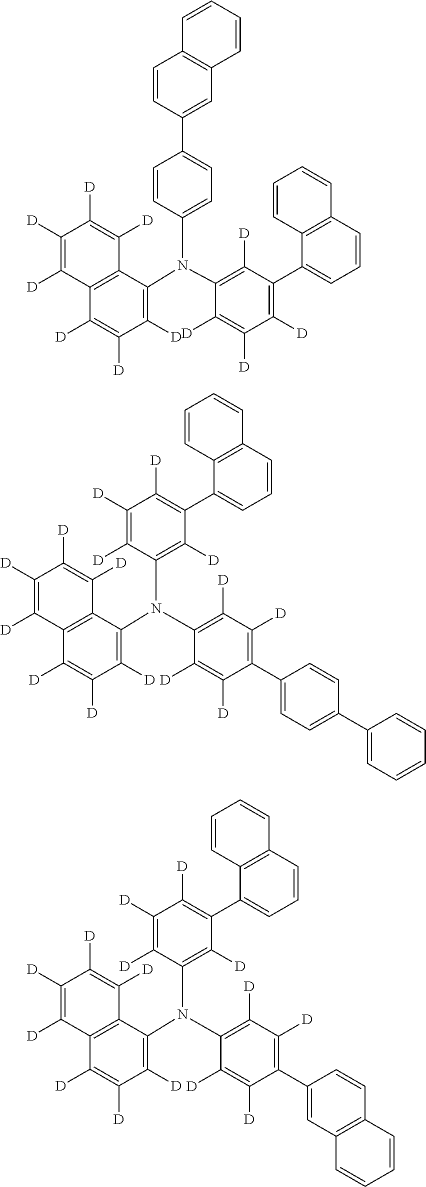

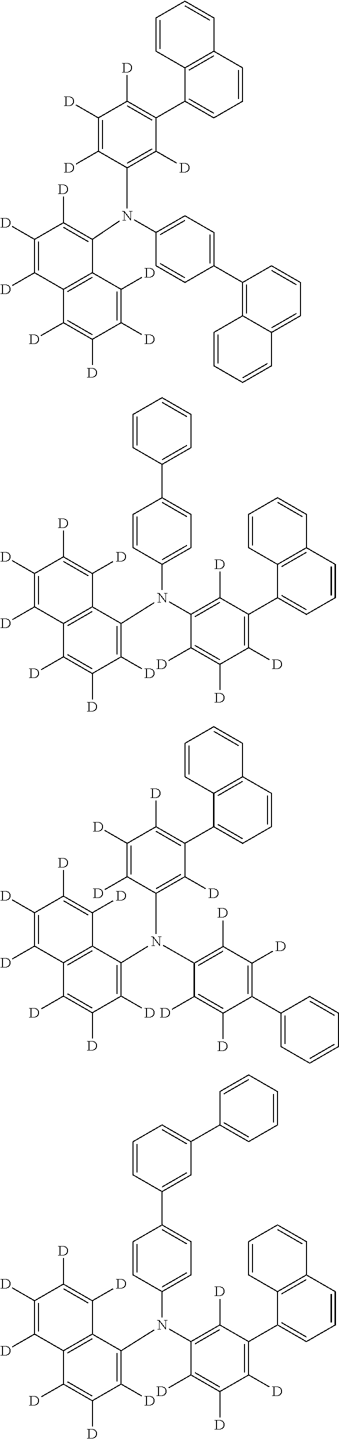

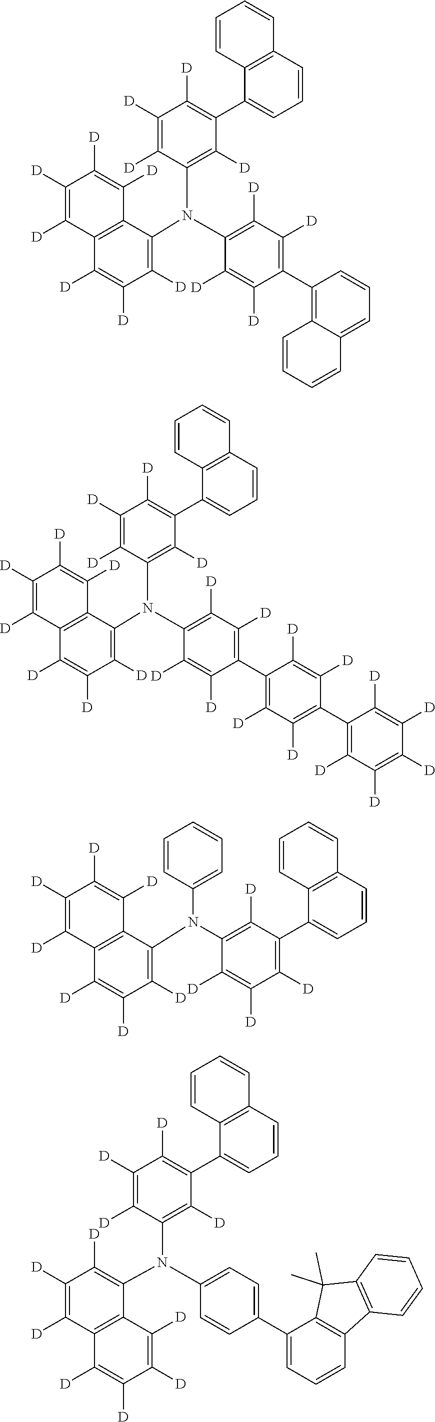

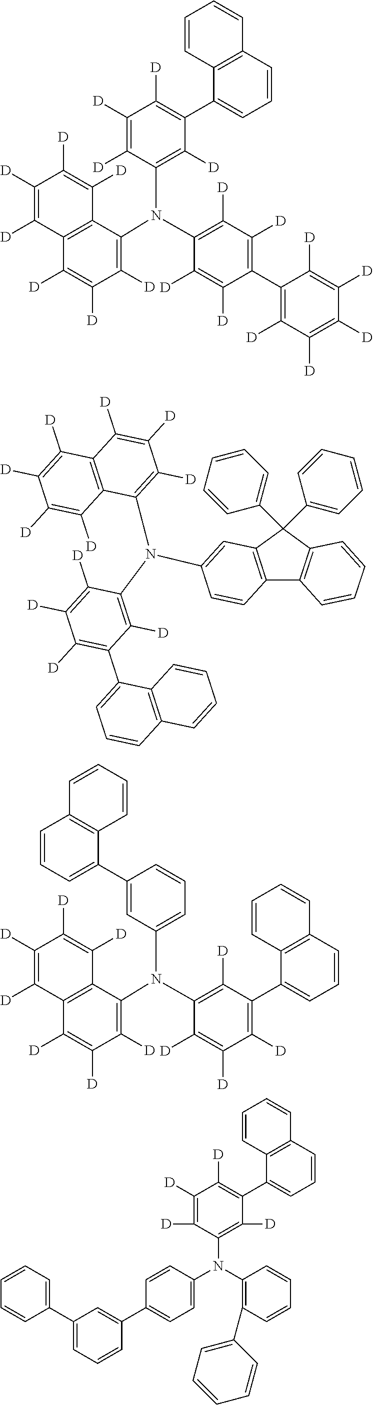

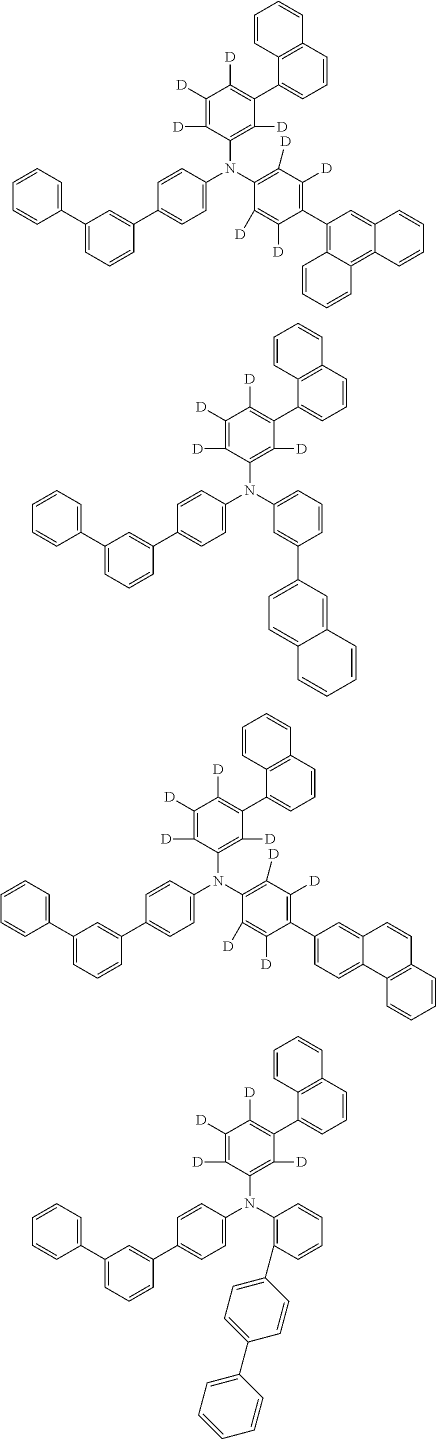

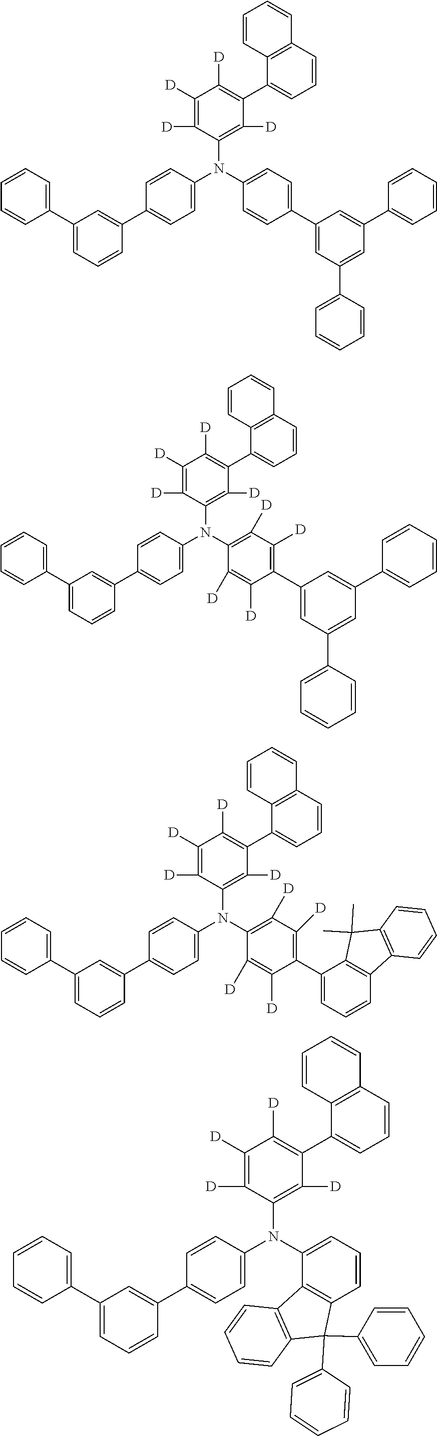

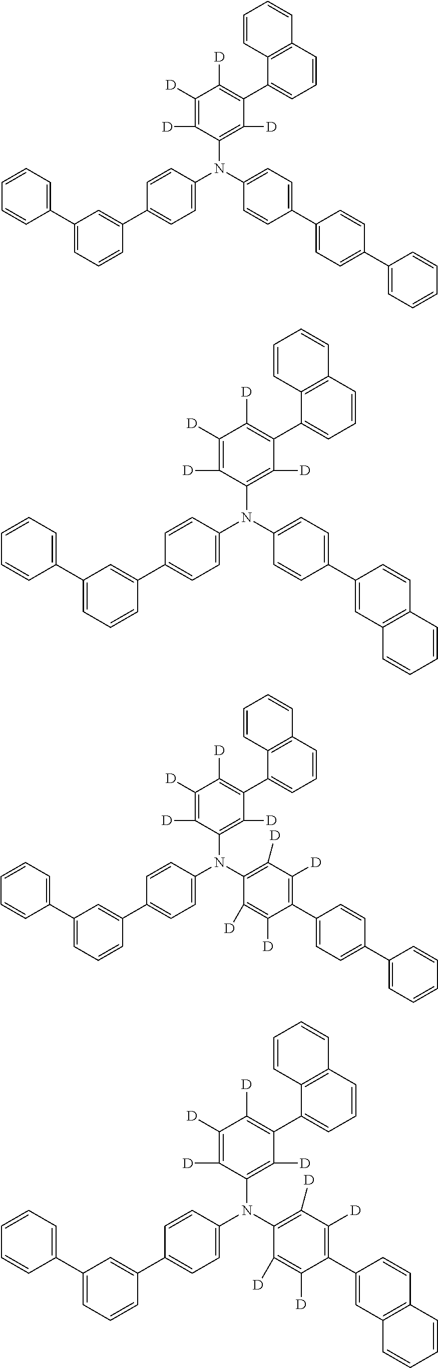 [SMILES: [2H]c1c([2H])c(-c2cccc3ccccc23)c([2H])c(N(c2ccc(-c3ccc(-c4ccccc4)cc3)cc2)c2ccc(-c3cccc(-c4ccccc4)c3)cc2)c1[2H] ; [2H]c1c([2H])c(-c2cccc3ccccc23)c([2H])c(N(c2ccc(-c3cccc(-c4ccccc4)c3)cc2)c2ccc(-c3ccc4ccccc4c3)cc2)c1[2H] ; [2H]c1c([2H])c(-c2cccc3ccccc23)c([2H])c(N(c2ccc(-c3cccc(-c4ccccc4)c3)cc2)c2c([2H])c([2H])c(-c3ccc(-c4ccccc4)cc3)c([2H])c2[2H])c1[2H] ; [2H]c1c([2H])c(-c2cccc3ccccc23)c([2H])c(N(c2ccc(-c3cccc(-c4ccccc4)c3)cc2)c2c([2H])c([2H])c(-c3ccc4ccccc4c3)c([2H])c2[2H])c1[2H]]

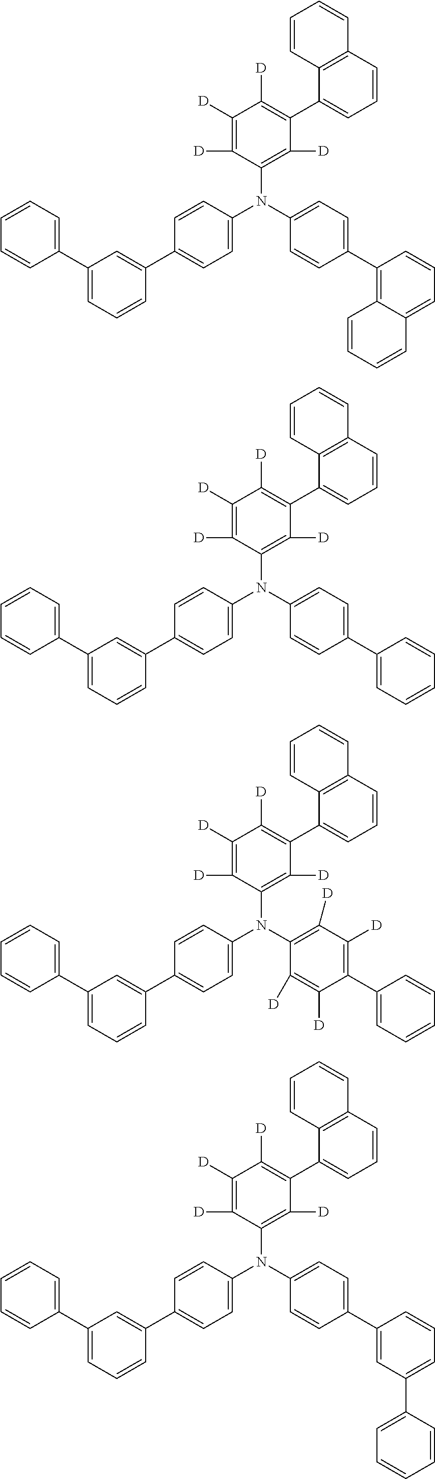

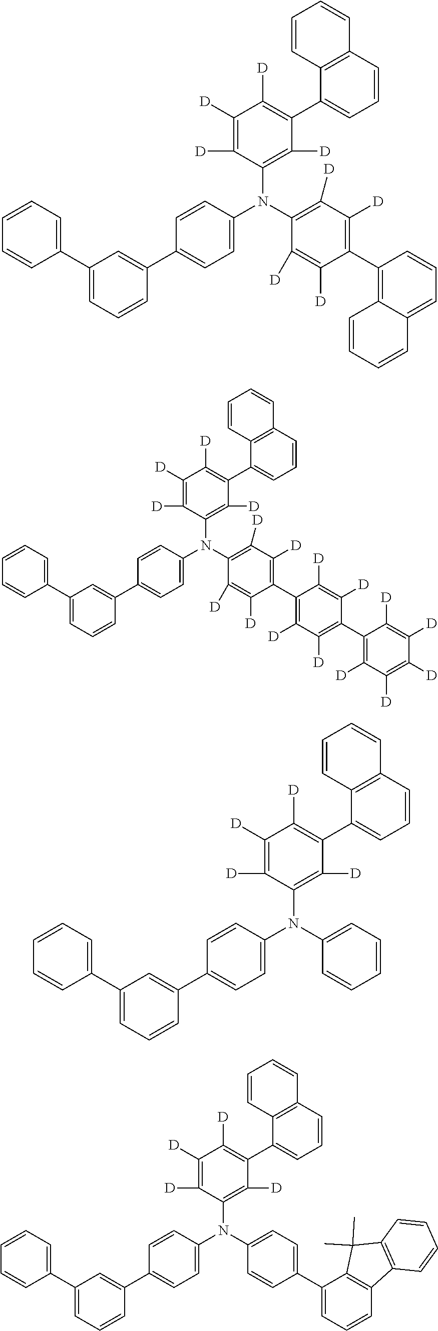

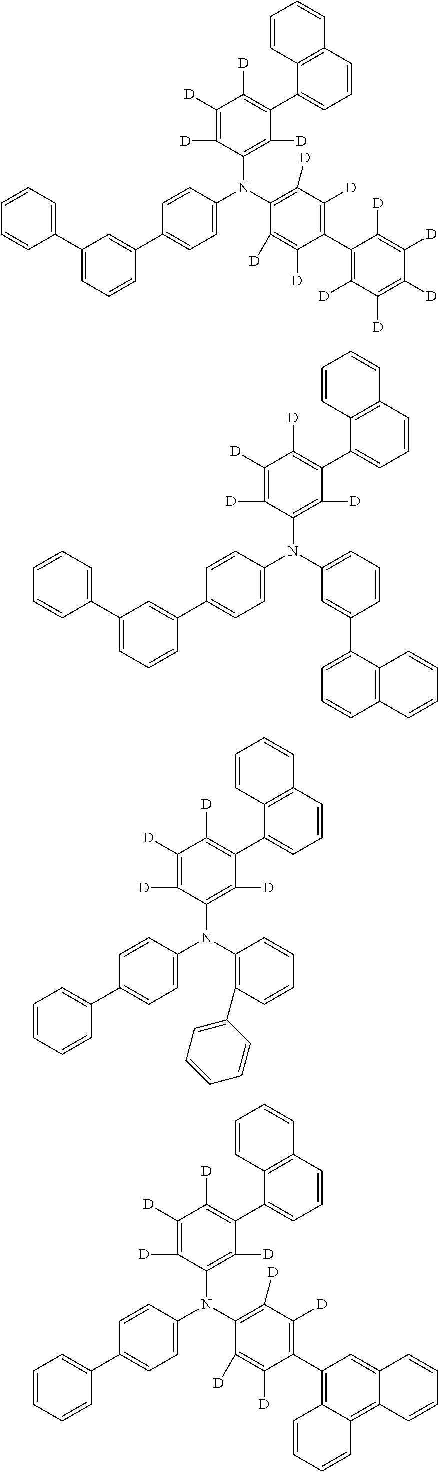

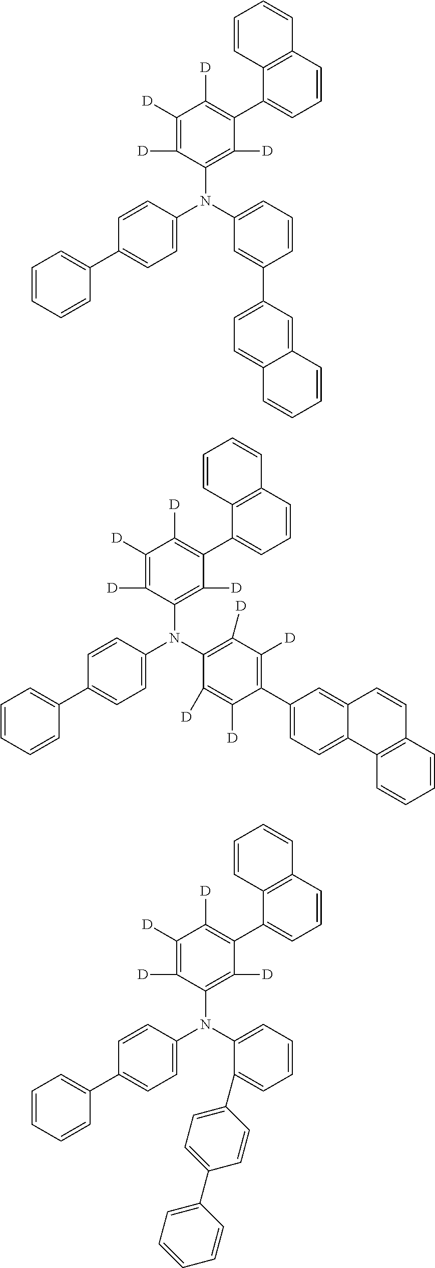 [SMILES: [2H]c1c([2H])c(-c2cccc3ccccc23)c([2H])c(N(c2ccc(-c3ccccc3)cc2)c2cccc(-c3ccc4ccccc4c3)c2)c1[2H] ; [2H]c1c([2H])c(-c2cccc3ccccc23)c([2H])c(N(c2ccc(-c3ccccc3)cc2)c2c([2H])c([2H])c(-c3ccc4c(ccc5ccccc54)c3)c([2H])c2[2H])c1[2H] ; [2H]c1c([2H])c(-c2cccc3ccccc23)c([2H])c(N(c2ccc(-c3ccccc3)cc2)c2ccccc2-c2ccc(-c3ccccc3)cc2)c1[2H]]

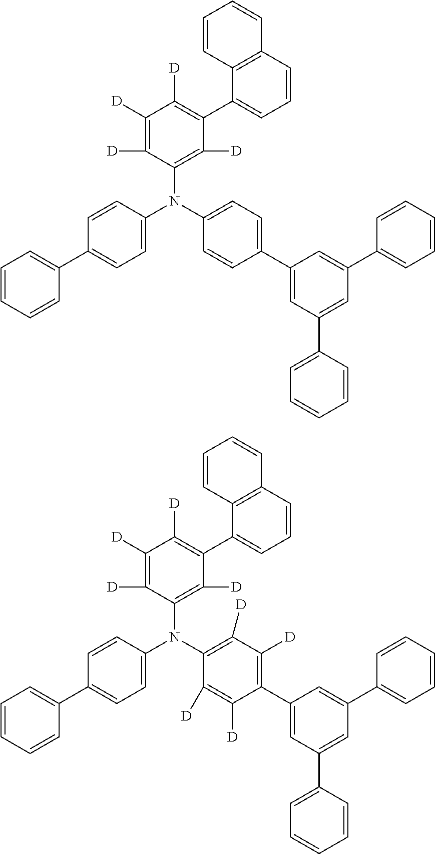 [SMILES: [2H]c1c([2H])c(-c2cccc3ccccc23)c([2H])c(N(c2ccc(-c3ccccc3)cc2)c2ccc(-c3cc(-c4ccccc4)cc(-c4ccccc4)c3)cc2)c1[2H] ; [2H]c1c([2H])c(-c2cccc3ccccc23)c([2H])c(N(c2ccc(-c3ccccc3)cc2)c2c([2H])c([2H])c(-c3cc(-c4ccccc4)cc(-c4ccccc4)c3)c([2H])c2[2H])c1[2H]]

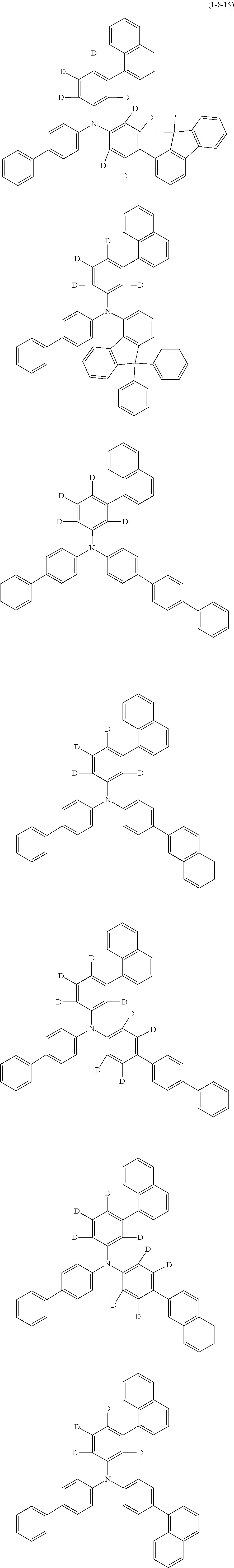

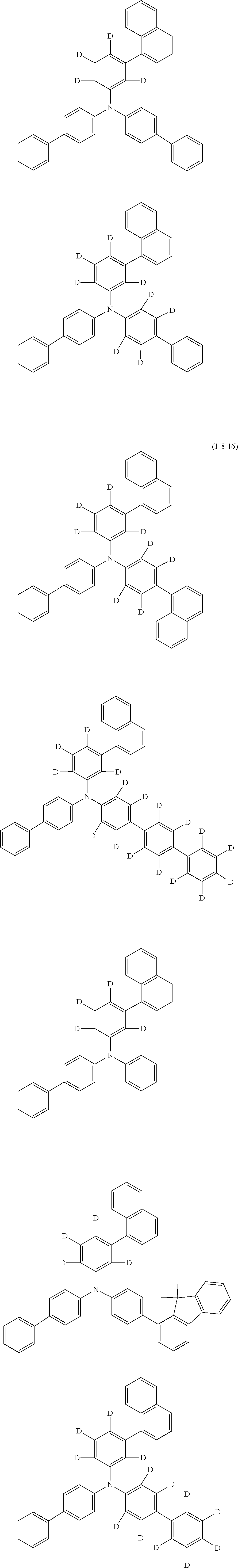

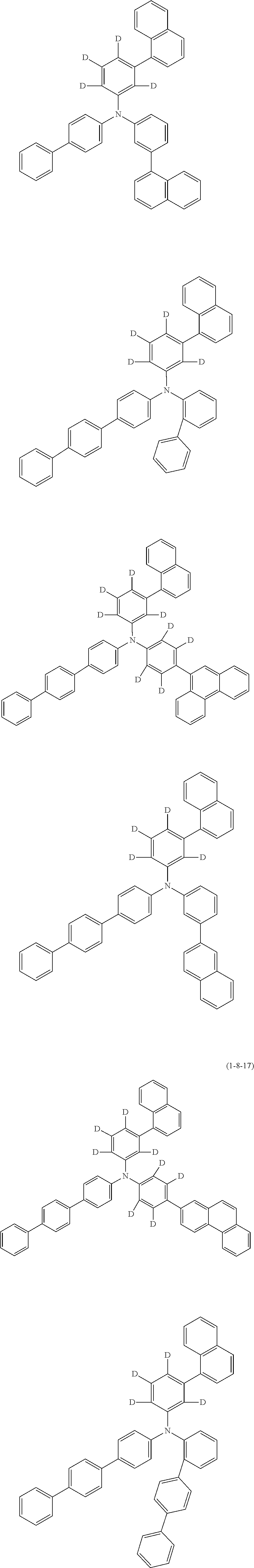

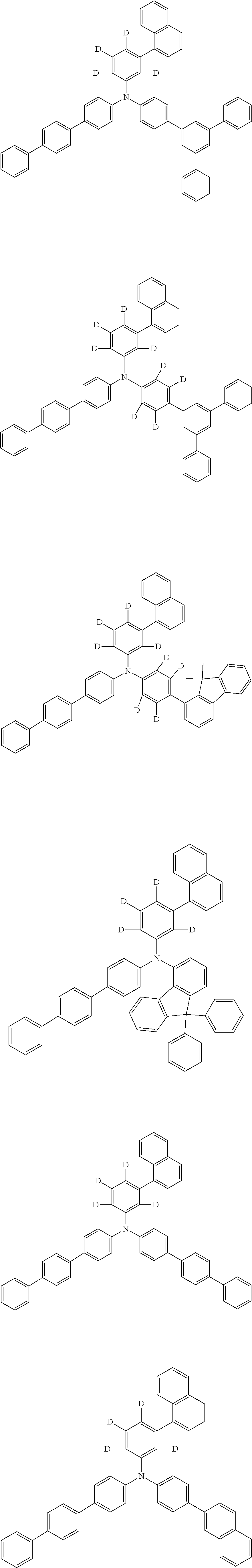

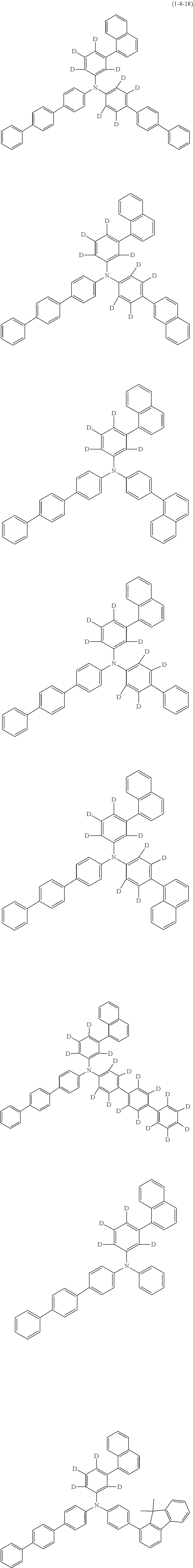

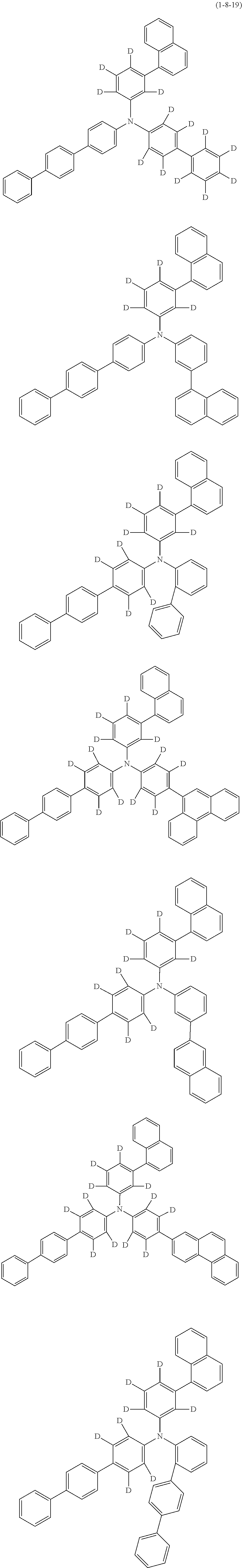

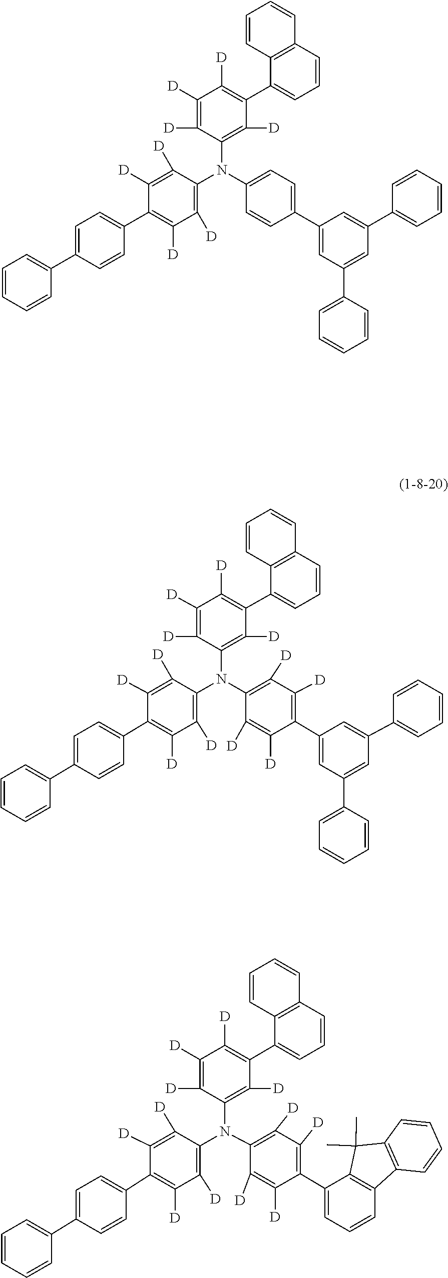 [SMILES: [2H]c1c([2H])c(-c2cccc3ccccc23)c([2H])c(N(c2ccc(-c3cc(-c4ccccc4)cc(-c4ccccc4)c3)cc2)c2c([2H])c([2H])c(-c3ccc(-c4ccccc4)cc3)c([2H])c2[2H])c1[2H] ; [2H]c1c([2H])c(-c2cccc3ccccc23)c([2H])c(N(c2c([2H])c([2H])c(-c3ccc(-c4ccccc4)cc3)c([2H])c2[2H])c2c([2H])c([2H])c(-c3cc(-c4ccccc4)cc(-c4ccccc4)c3)c([2H])c2[2H])c1[2H] ; [2H]c1c([2H])c(-c2cccc3ccccc23)c([2H])c(N(c2c([2H])c([2H])c(-c3ccc(-c4ccccc4)cc3)c([2H])c2[2H])c2c([2H])c([2H])c(-c3cccc4c3C(C)(C)c3ccccc3-4)c([2H])c2[2H])c1[2H]]

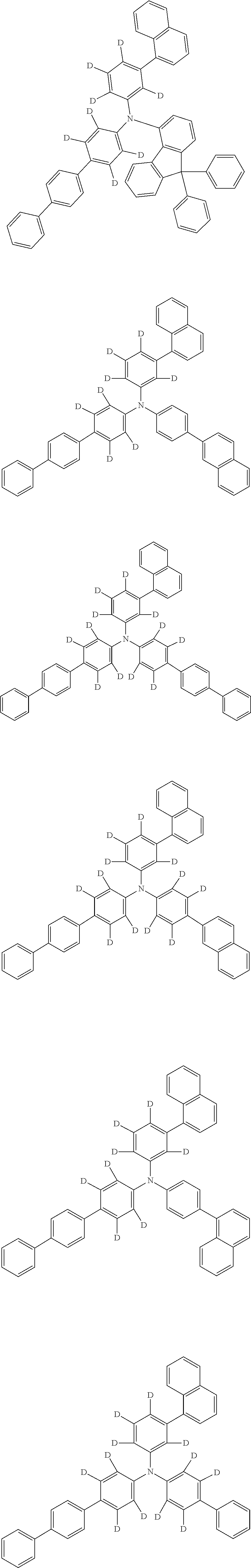

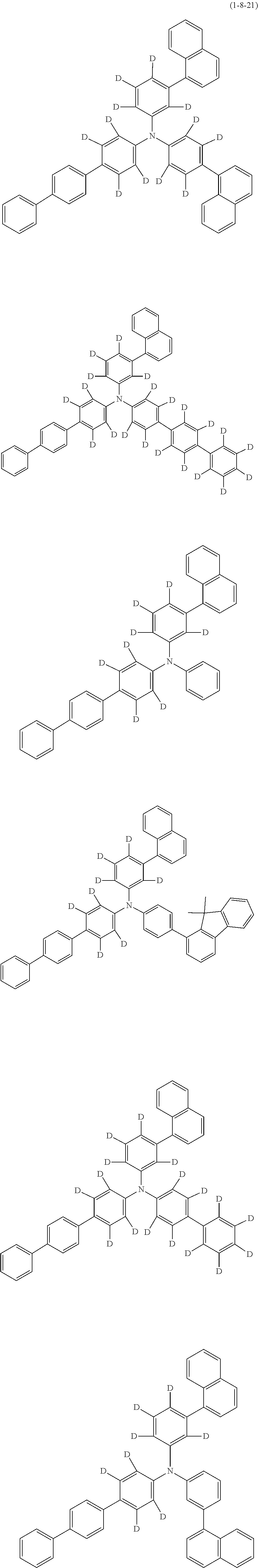

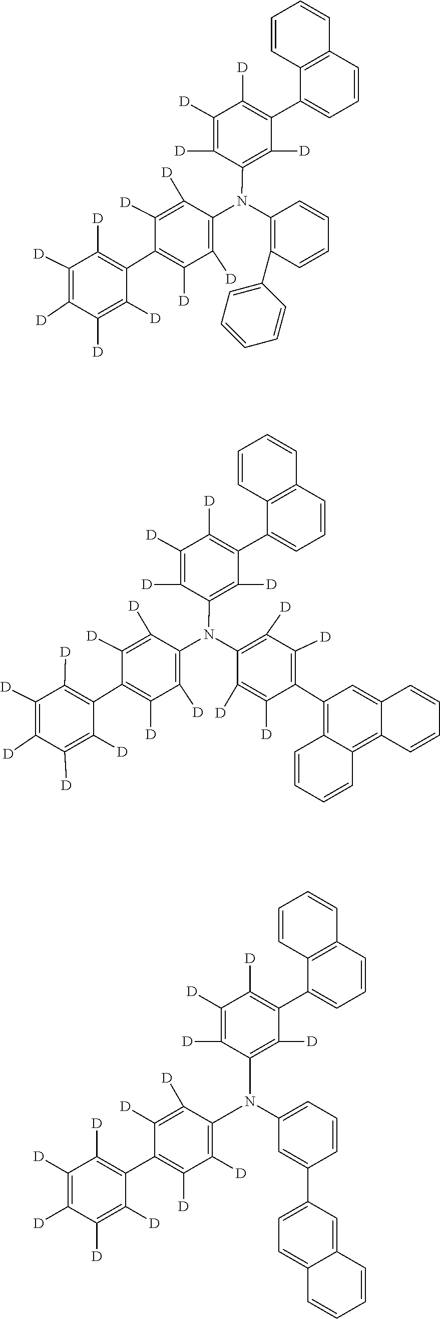 [SMILES: [2H]c1c([2H])c([2H])c(-c2c([2H])c([2H])c(N(c3ccccc3-c3ccccc3)c3c([2H])c([2H])c([2H])c(-c4cccc5ccccc45)c3[2H])c([2H])c2[2H])c([2H])c1[2H] ; [2H]c1c([2H])c([2H])c(-c2c([2H])c([2H])c(N(c3c([2H])c([2H])c(-c4cc5ccccc5c5ccccc45)c([2H])c3[2H])c3c([2H])c([2H])c([2H])c(-c4cccc5ccccc45)c3[2H])c([2H])c2[2H])c([2H])c1[2H] ; [2H]c1c([2H])c([2H])c(-c2c([2H])c([2H])c(N(c3cccc(-c4ccc5ccccc5c4)c3)c3c([2H])c([2H])c([2H])c(-c4cccc5ccccc45)c3[2H])c([2H])c2[2H])c([2H])c1[2H]]

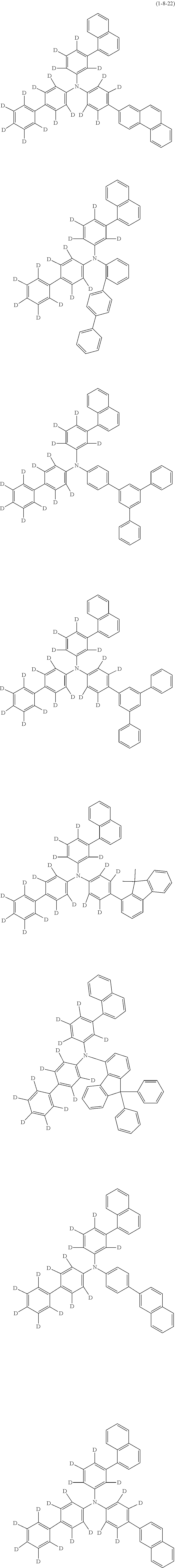

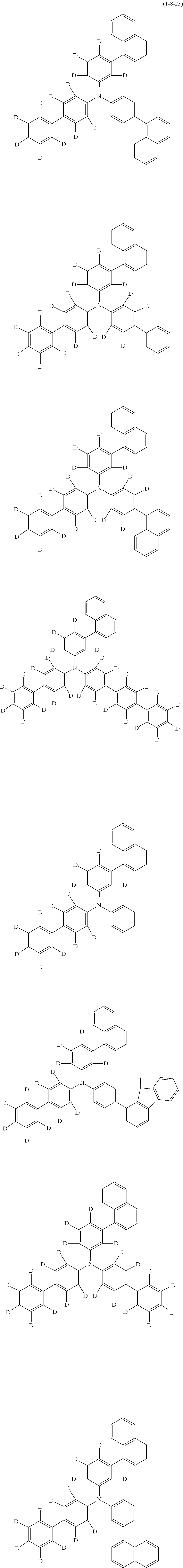

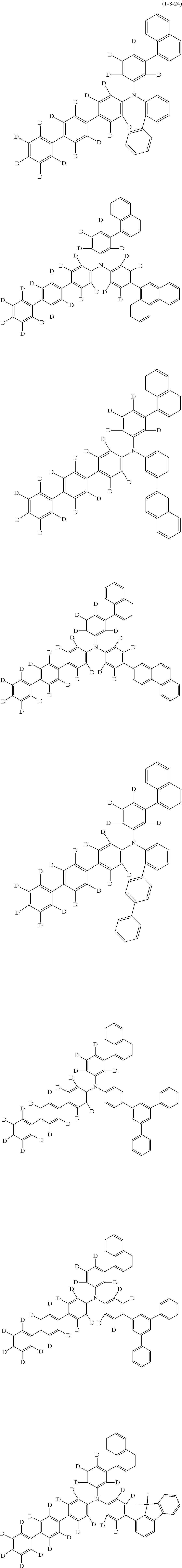

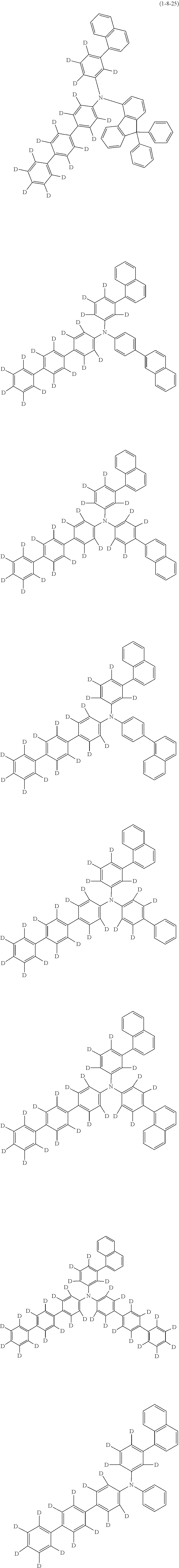

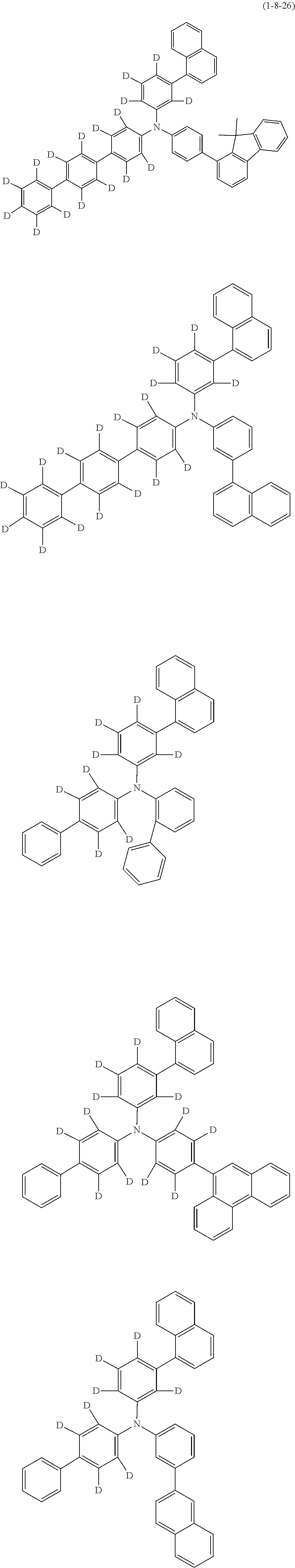

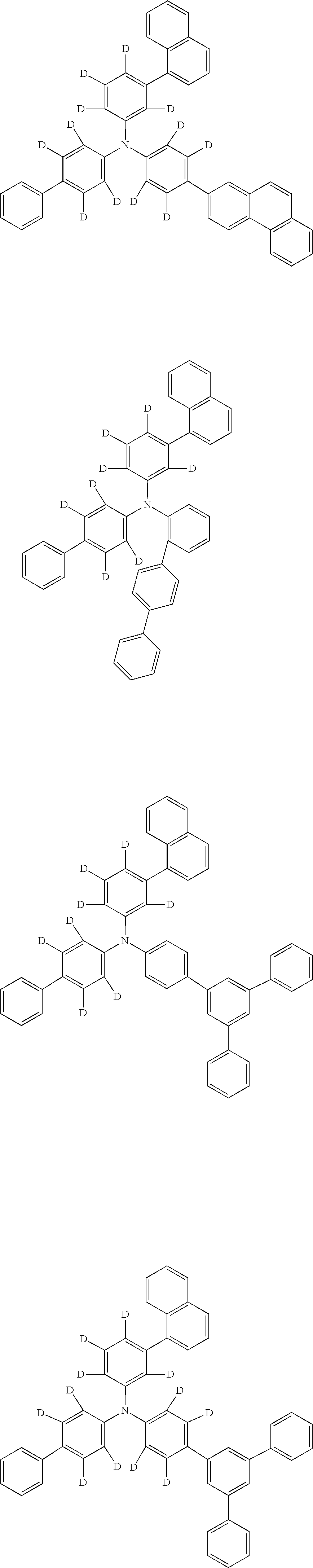

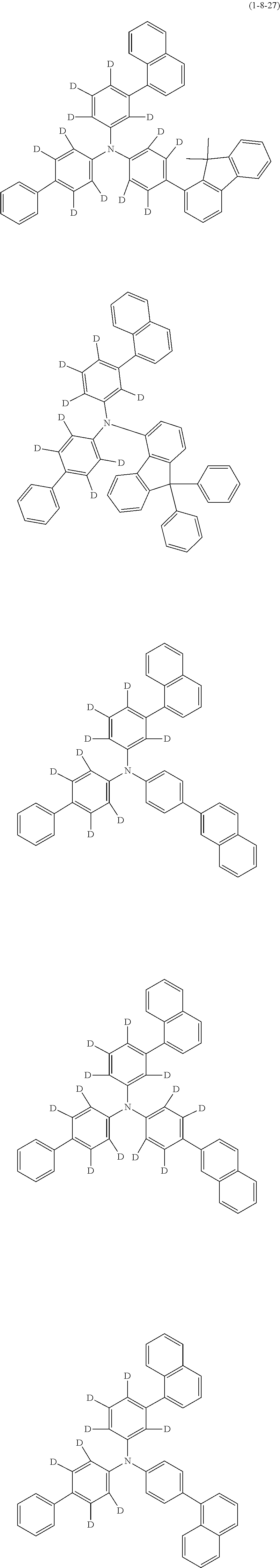

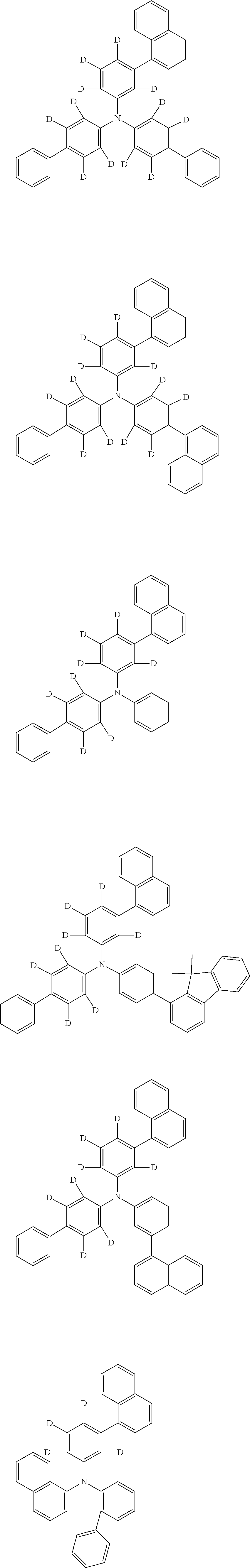

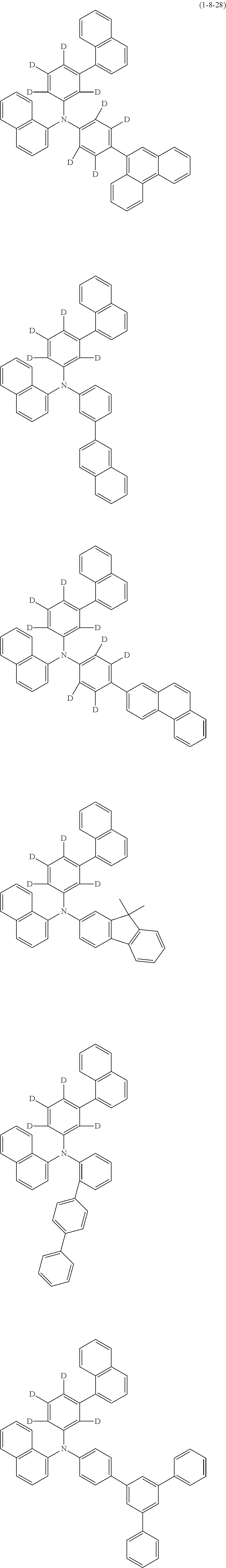

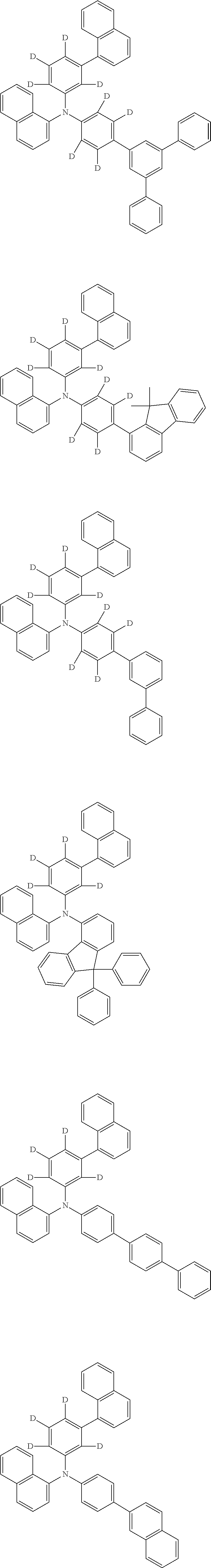

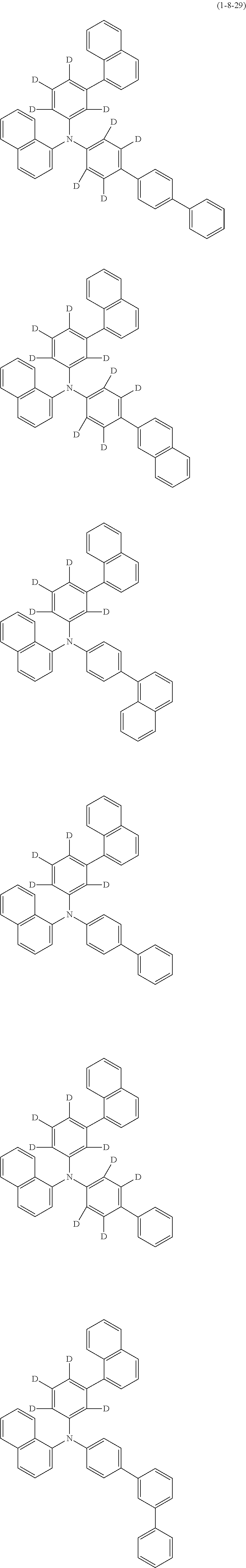

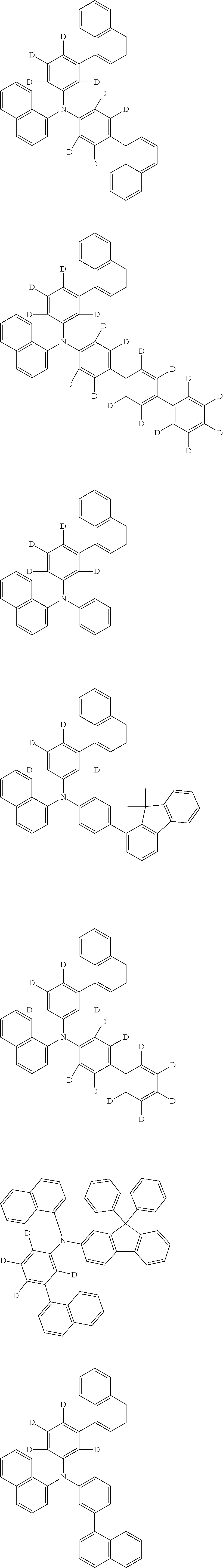

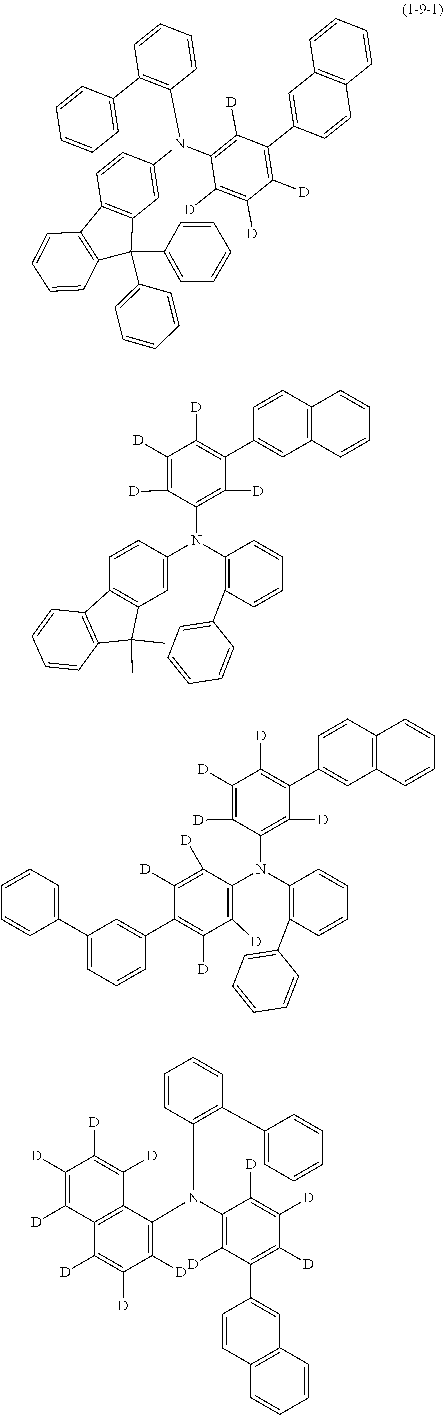 [SMILES: [2H]c1c([2H])c(-c2ccc3ccccc3c2)c([2H])c(N(c2ccc3c(c2)C(c2ccccc2)(c2ccccc2)c2ccccc2-3)c2ccccc2-c2ccccc2)c1[2H] ; [2H]c1c([2H])c(-c2ccc3ccccc3c2)c([2H])c(N(c2ccc3c(c2)C(C)(C)c2ccccc2-3)c2ccccc2-c2ccccc2)c1[2H] ; [2H]c1c([2H])c(-c2ccc3ccccc3c2)c([2H])c(N(c2ccccc2-c2ccccc2)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2ccccc2-c2ccccc2)c2c([2H])c([2H])c([2H])c3c([2H])c([2H])c([2H])c([2H])c23)c1[2H]]

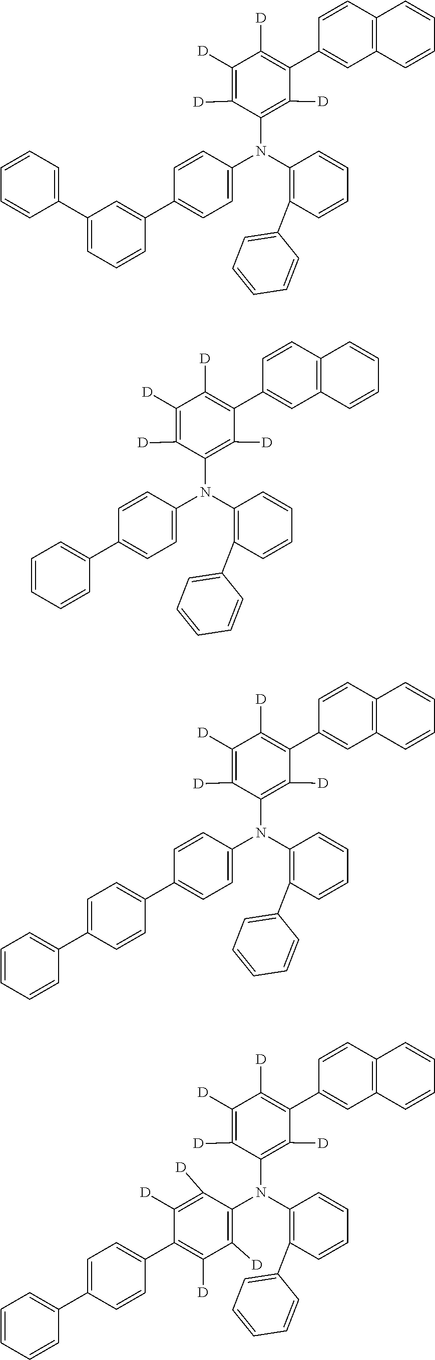 [SMILES: [2H]c1c([2H])c(-c2ccc3ccccc3c2)c([2H])c(N(c2ccc(-c3cccc(-c4ccccc4)c3)cc2)c2ccccc2-c2ccccc2)c1[2H] ; [2H]c1c([2H])c(-c2ccc3ccccc3c2)c([2H])c(N(c2ccc(-c3ccccc3)cc2)c2ccccc2-c2ccccc2)c1[2H] ; [2H]c1c([2H])c(-c2ccc3ccccc3c2)c([2H])c(N(c2ccc(-c3ccc(-c4ccccc4)cc3)cc2)c2ccccc2-c2ccccc2)c1[2H] ; [2H]c1c([2H])c(-c2ccc3ccccc3c2)c([2H])c(N(c2ccccc2-c2ccccc2)c2c([2H])c([2H])c(-c3ccc(-c4ccccc4)cc3)c([2H])c2[2H])c1[2H]]

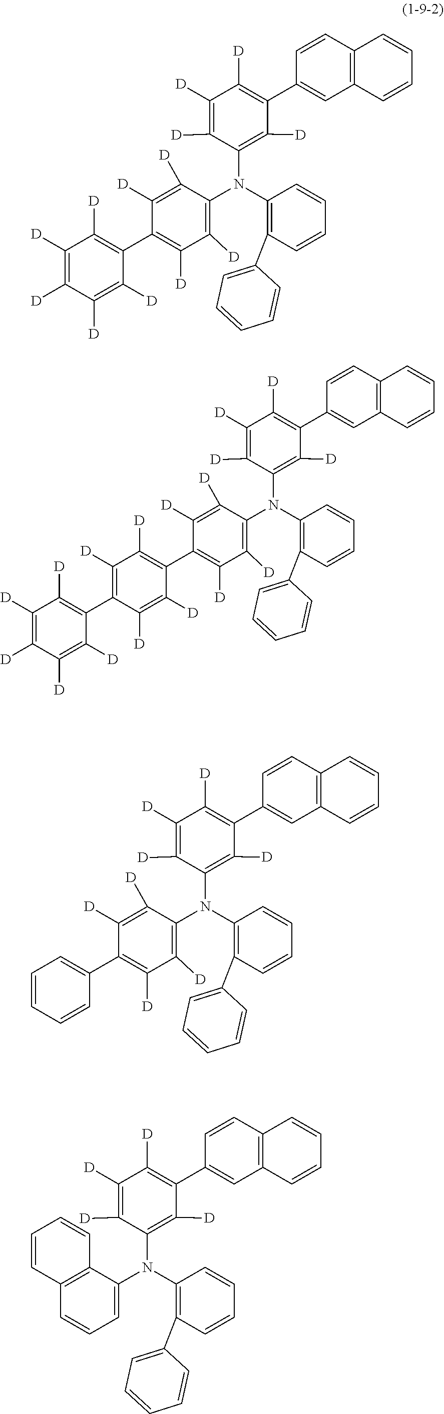

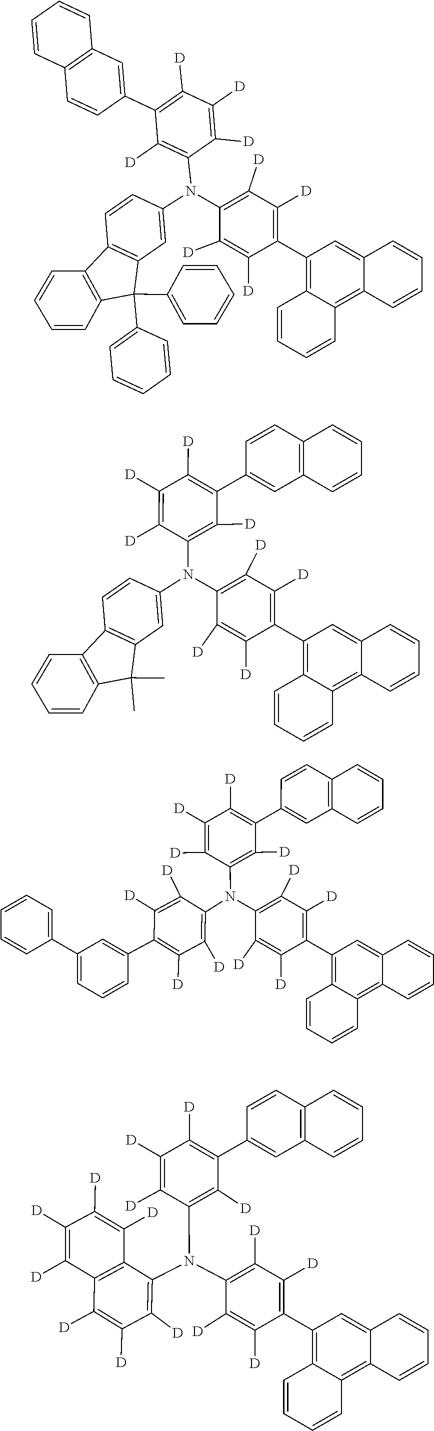

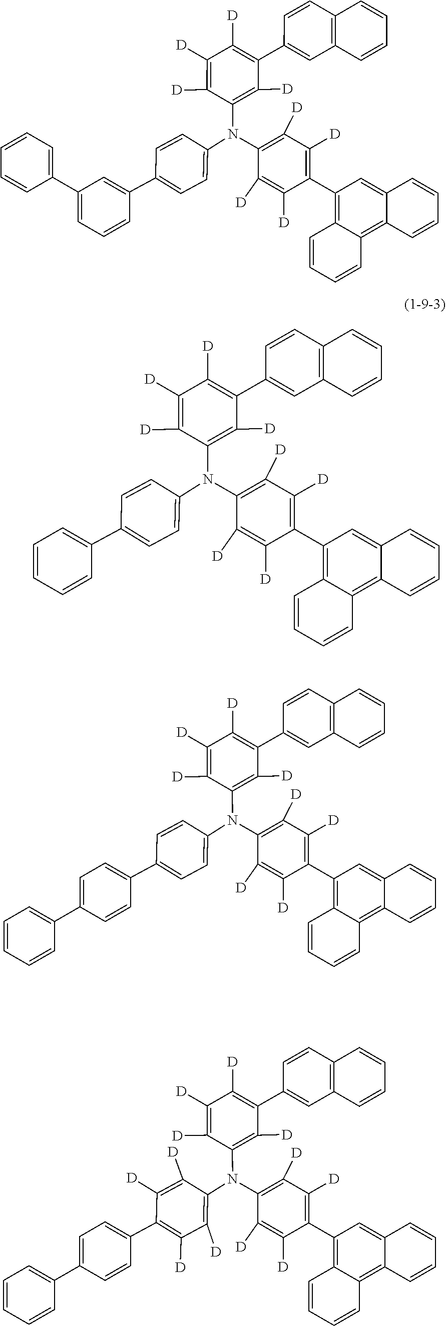

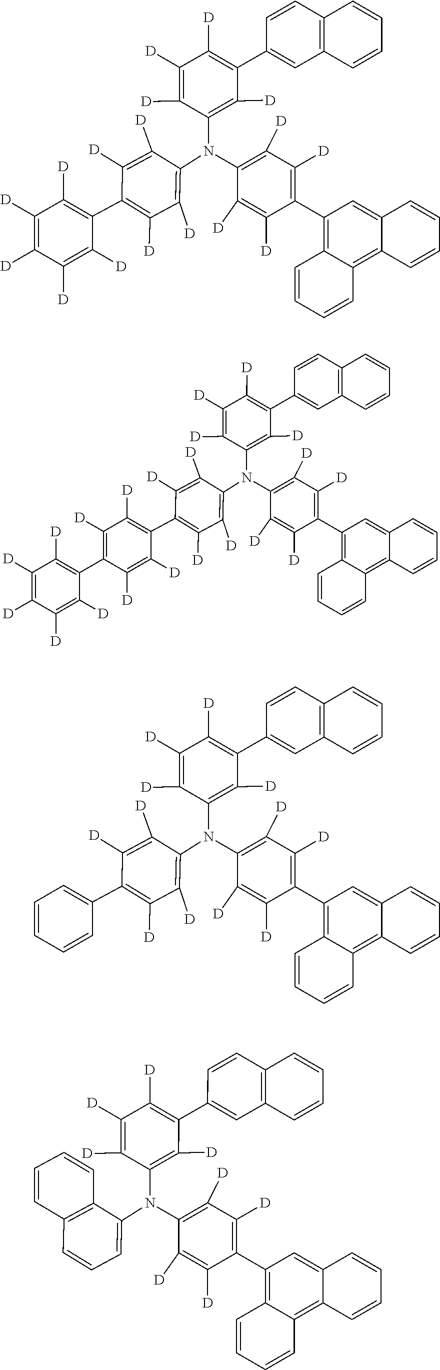

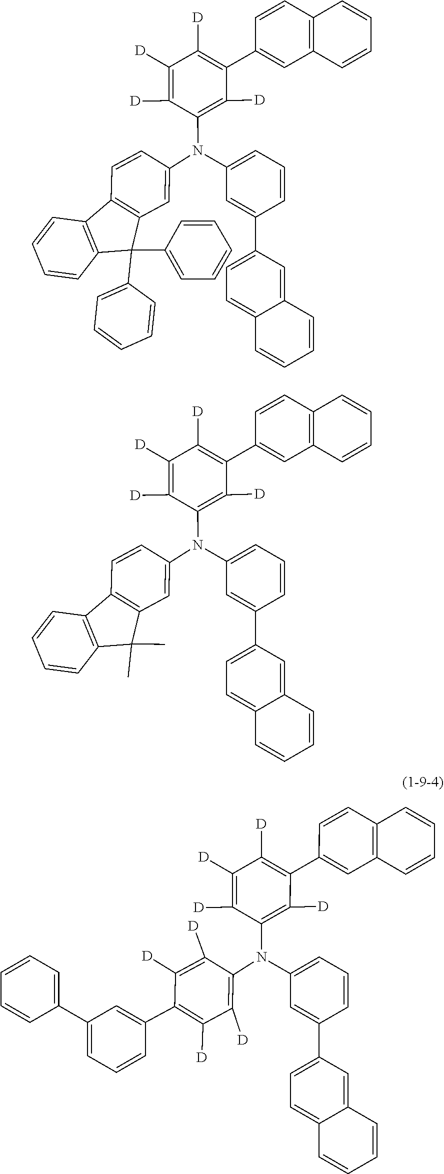

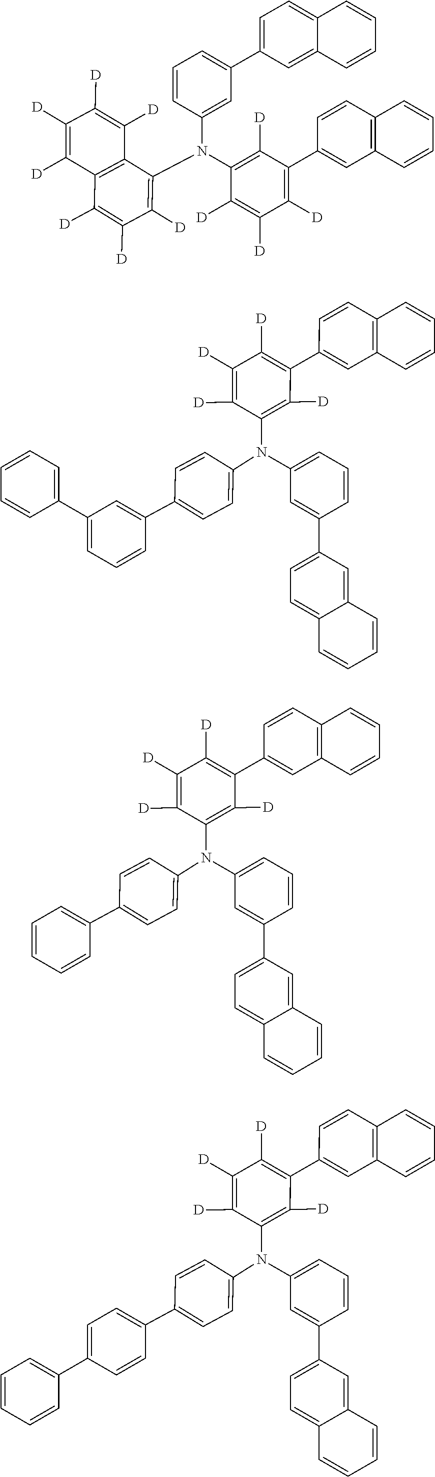

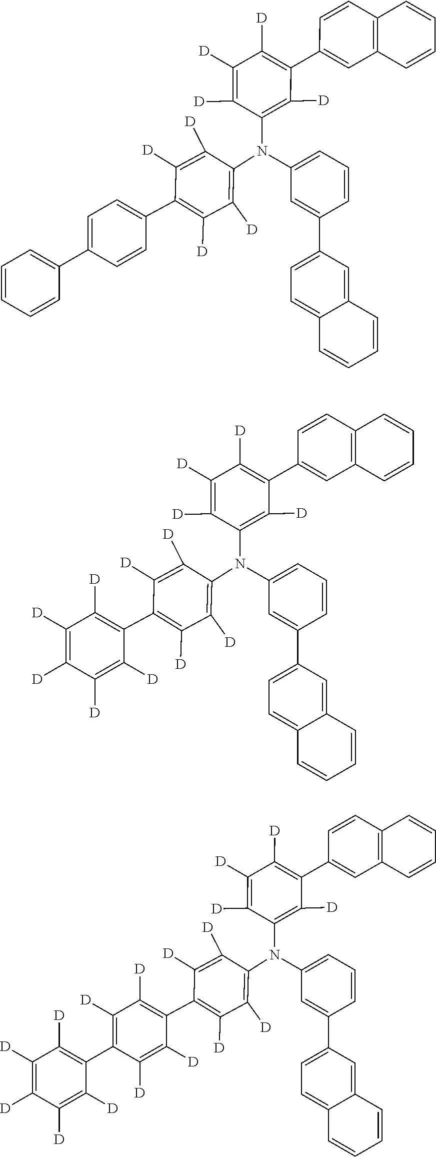

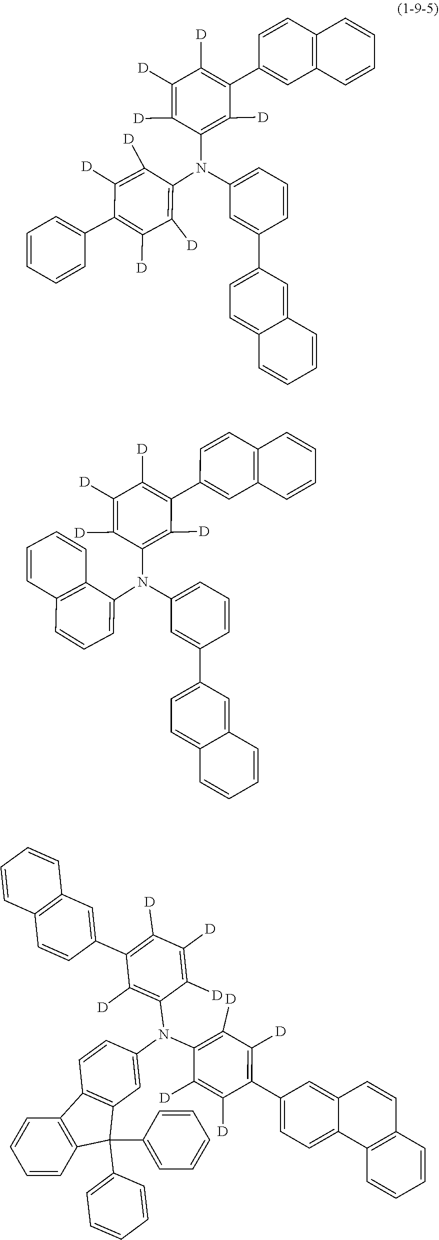 [SMILES: [2H]c1c([2H])c(-c2ccc3ccccc3c2)c([2H])c(N(c2cccc(-c3ccc4ccccc4c3)c2)c2c([2H])c([2H])c(-c3ccccc3)c([2H])c2[2H])c1[2H] ; [2H]c1c([2H])c(-c2ccc3ccccc3c2)c([2H])c(N(c2cccc(-c3ccc4ccccc4c3)c2)c2cccc3ccccc23)c1[2H] ; [2H]c1c([2H])c(-c2ccc3ccccc3c2)c([2H])c(N(c2ccc3c(c2)C(c2ccccc2)(c2ccccc2)c2ccccc2-3)c2c([2H])c([2H])c(-c3ccc4c(ccc5ccccc54)c3)c([2H])c2[2H])c1[2H]]

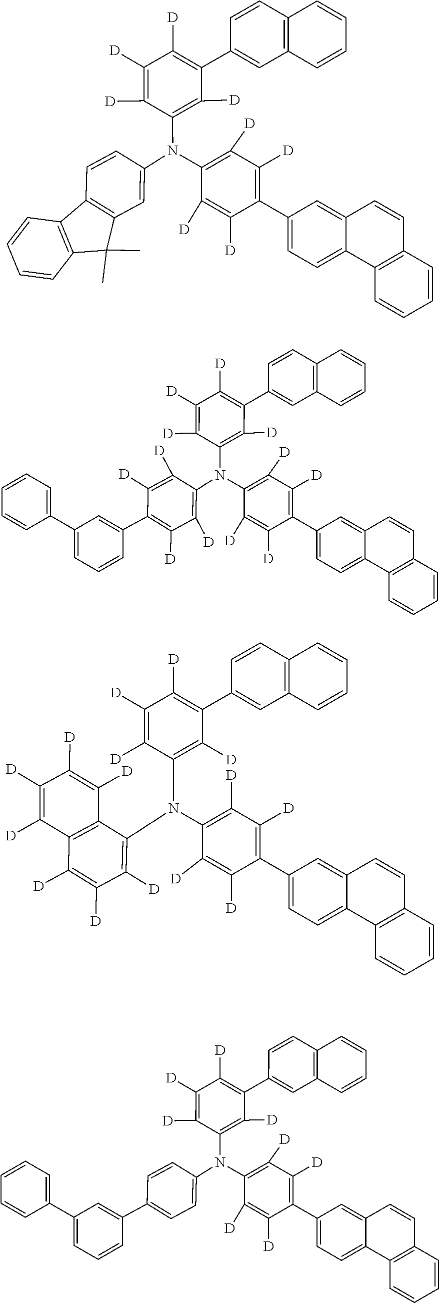

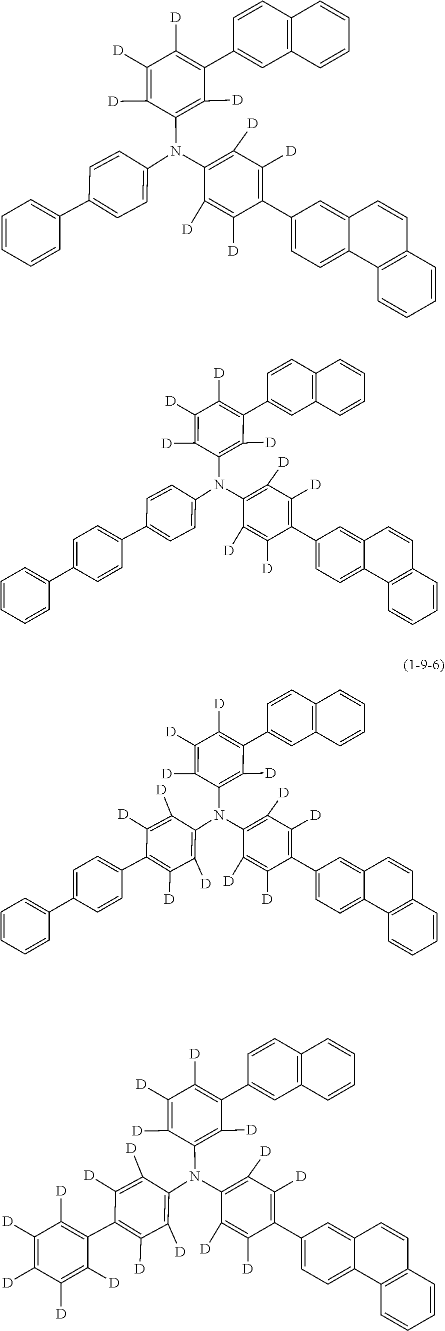

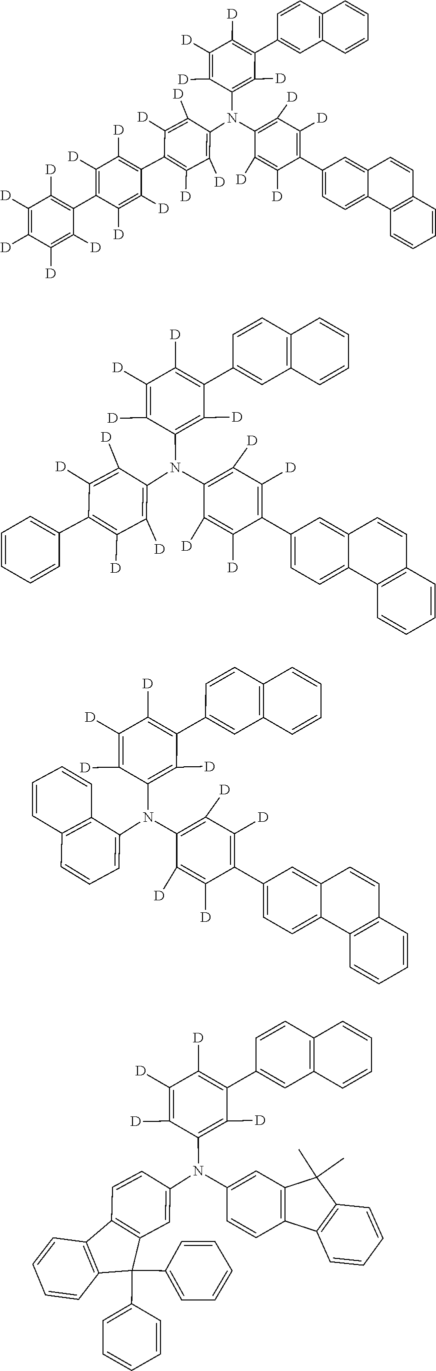

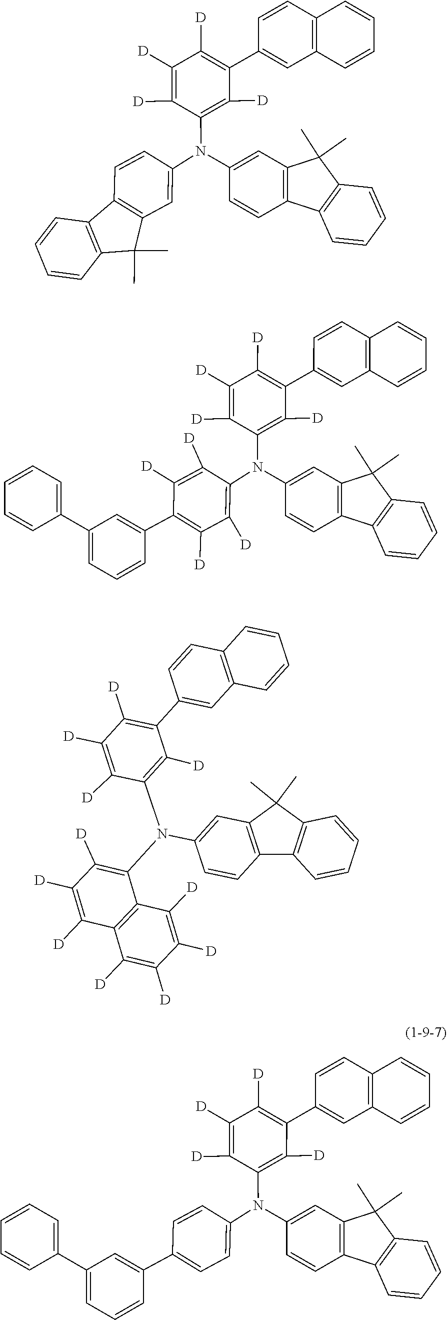 [SMILES: [2H]c1c([2H])c(-c2ccc3ccccc3c2)c([2H])c(N(c2ccc3c(c2)C(C)(C)c2ccccc2-3)c2ccc3c(c2)C(C)(C)c2ccccc2-3)c1[2H] ; [2H]c1c([2H])c(-c2ccc3ccccc3c2)c([2H])c(N(c2ccc3c(c2)C(C)(C)c2ccccc2-3)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2ccc3c(c2)C(C)(C)c2ccccc2-3)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc(-c3cccc(-c4ccccc4)c3)cc2)c2ccc3c(c2)C(C)(C)c2ccccc2-3)c1[2H]]

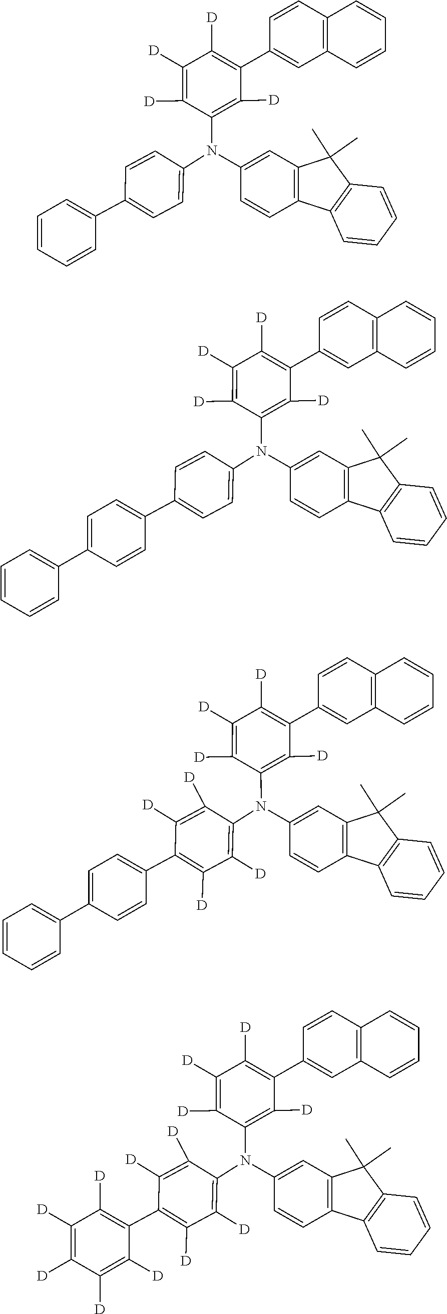 [SMILES: [2H]c1c([2H])c(-c2ccc3ccccc3c2)c([2H])c(N(c2ccc(-c3ccccc3)cc2)c2ccc3c(c2)C(C)(C)c2ccccc2-3)c1[2H] ; [2H]c1c([2H])c(-c2ccc3ccccc3c2)c([2H])c(N(c2ccc(-c3ccc(-c4ccccc4)cc3)cc2)c2ccc3c(c2)C(C)(C)c2ccccc2-3)c1[2H] ; [2H]c1c([2H])c(-c2ccc3ccccc3c2)c([2H])c(N(c2ccc3c(c2)C(C)(C)c2ccccc2-3)c2c([2H])c([2H])c(-c3ccc(-c4ccccc4)cc3)c([2H])c2[2H])c1[2H] ; [2H]c1c([2H])c([2H])c(-c2c([2H])c([2H])c(N(c3ccc4c(c3)C(C)(C)c3ccccc3-4)c3c([2H])c([2H])c([2H])c(-c4ccc5ccccc5c4)c3[2H])c([2H])c2[2H])c([2H])c1[2H]]

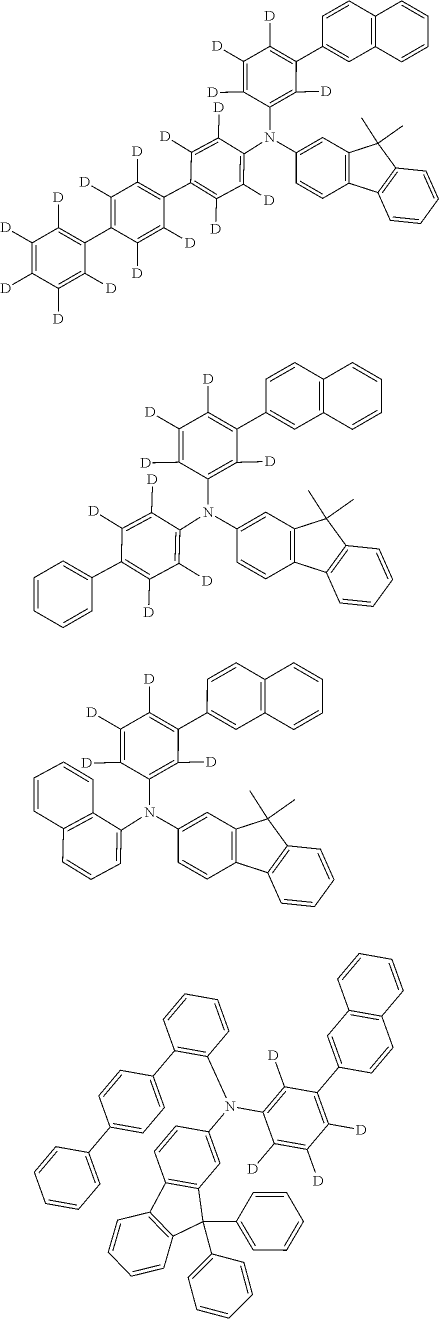

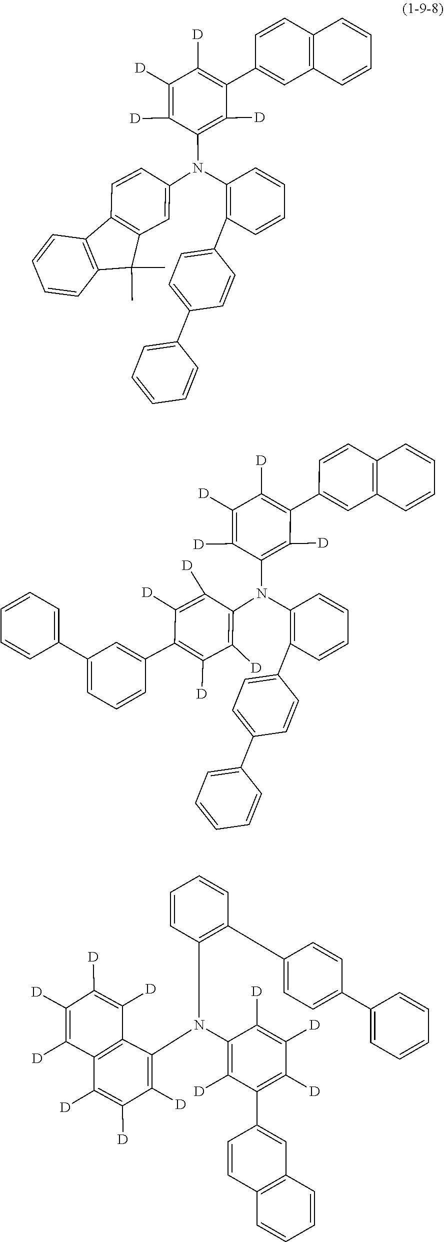 [SMILES: [2H]c1c([2H])c(-c2ccc3ccccc3c2)c([2H])c(N(c2ccc3c(c2)C(C)(C)c2ccccc2-3)c2ccccc2-c2ccc(-c3ccccc3)cc2)c1[2H] ; [2H]c1c([2H])c(-c2ccc3ccccc3c2)c([2H])c(N(c2ccccc2-c2ccc(-c3ccccc3)cc2)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2ccccc2-c2ccc(-c3ccccc3)cc2)c2c([2H])c([2H])c([2H])c3c([2H])c([2H])c([2H])c([2H])c23)c1[2H]]

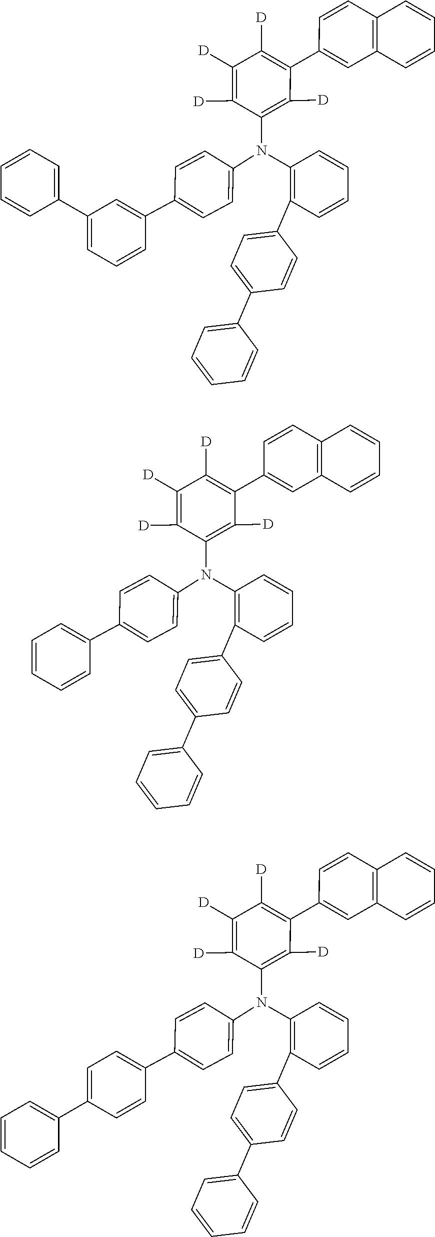 [SMILES: [2H]c1c([2H])c(-c2ccc3ccccc3c2)c([2H])c(N(c2ccc(-c3cccc(-c4ccccc4)c3)cc2)c2ccccc2-c2ccc(-c3ccccc3)cc2)c1[2H] ; [2H]c1c([2H])c(-c2ccc3ccccc3c2)c([2H])c(N(c2ccc(-c3ccccc3)cc2)c2ccccc2-c2ccc(-c3ccccc3)cc2)c1[2H] ; [2H]c1c([2H])c(-c2ccc3ccccc3c2)c([2H])c(N(c2ccc(-c3ccc(-c4ccccc4)cc3)cc2)c2ccccc2-c2ccc(-c3ccccc3)cc2)c1[2H]]

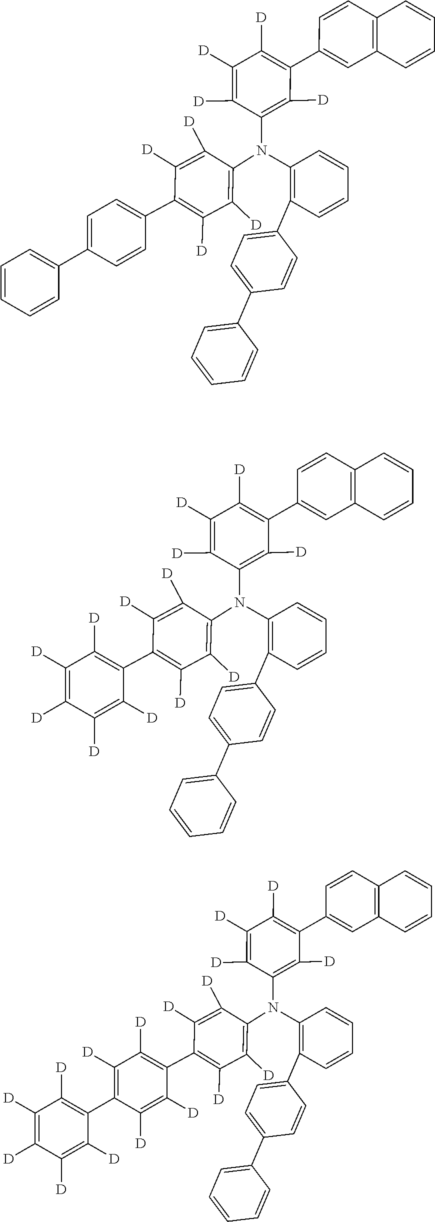 [SMILES: [2H]c1c([2H])c(-c2ccc3ccccc3c2)c([2H])c(N(c2ccccc2-c2ccc(-c3ccccc3)cc2)c2c([2H])c([2H])c(-c3ccc(-c4ccccc4)cc3)c([2H])c2[2H])c1[2H] ; [2H]c1c([2H])c([2H])c(-c2c([2H])c([2H])c(N(c3ccccc3-c3ccc(-c4ccccc4)cc3)c3c([2H])c([2H])c([2H])c(-c4ccc5ccccc5c4)c3[2H])c([2H])c2[2H])c([2H])c1[2H] ; [2H]c1c([2H])c([2H])c(-c2c([2H])c([2H])c(-c3c([2H])c([2H])c(N(c4ccccc4-c4ccc(-c5ccccc5)cc4)c4c([2H])c([2H])c([2H])c(-c5ccc6ccccc6c5)c4[2H])c([2H])c3[2H])c([2H])c2[2H])c([2H])c1[2H]]

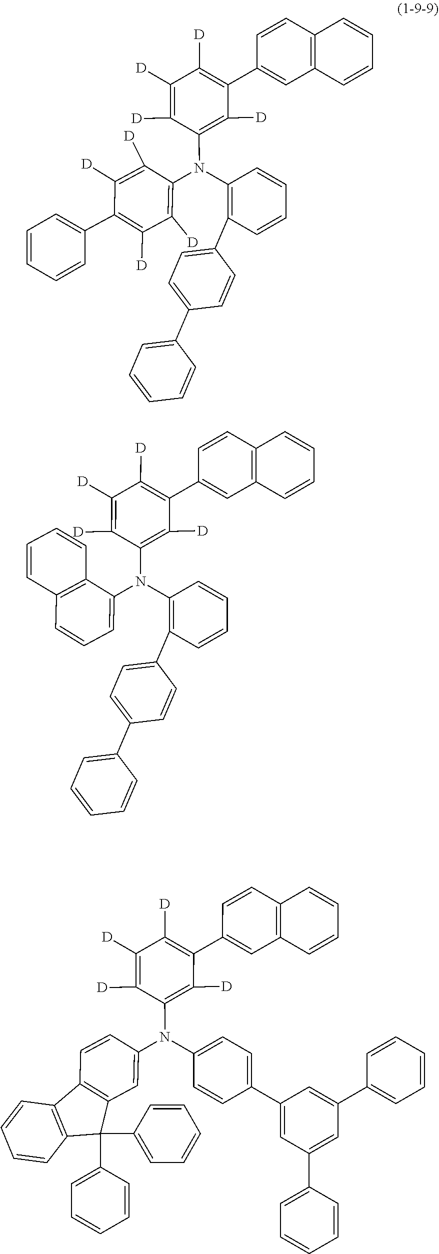 [SMILES: [2H]c1c([2H])c(-c2ccc3ccccc3c2)c([2H])c(N(c2ccccc2-c2ccc(-c3ccccc3)cc2)c2c([2H])c([2H])c(-c3ccccc3)c([2H])c2[2H])c1[2H] ; [2H]c1c([2H])c(-c2ccc3ccccc3c2)c([2H])c(N(c2ccccc2-c2ccc(-c3ccccc3)cc2)c2cccc3ccccc23)c1[2H] ; [2H]c1c([2H])c(-c2ccc3ccccc3c2)c([2H])c(N(c2ccc(-c3cc(-c4ccccc4)cc(-c4ccccc4)c3)cc2)c2ccc3c(c2)C(c2ccccc2)(c2ccccc2)c2ccccc2-3)c1[2H]]

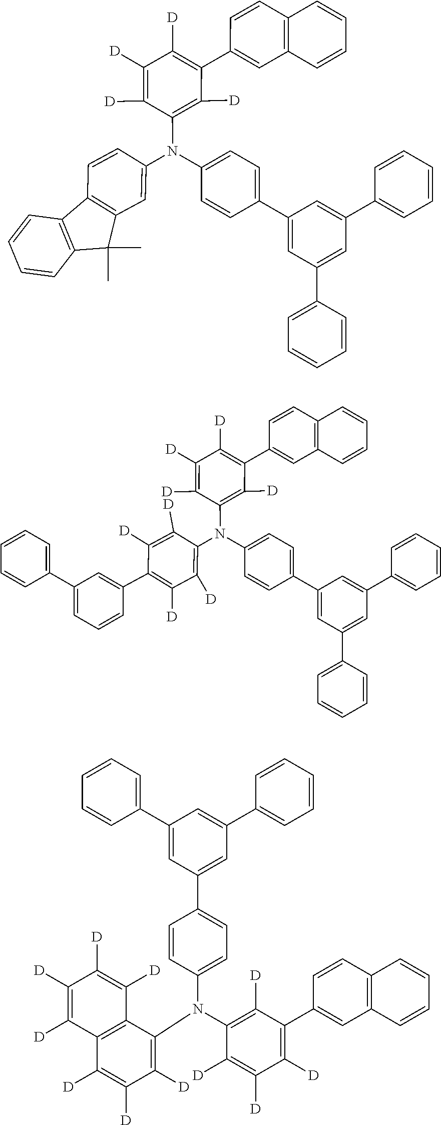

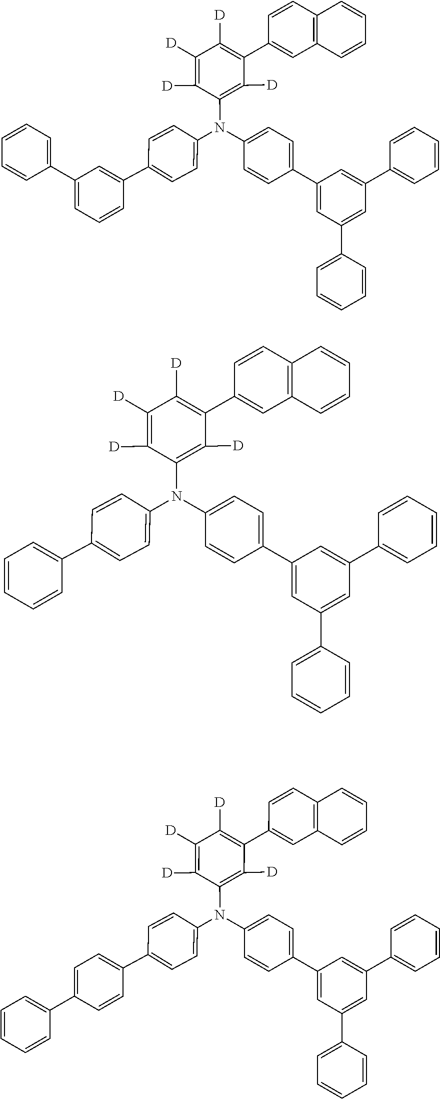 [SMILES: [2H]c1c([2H])c(-c2ccc3ccccc3c2)c([2H])c(N(c2ccc(-c3cccc(-c4ccccc4)c3)cc2)c2ccc(-c3cc(-c4ccccc4)cc(-c4ccccc4)c3)cc2)c1[2H] ; [2H]c1c([2H])c(-c2ccc3ccccc3c2)c([2H])c(N(c2ccc(-c3ccccc3)cc2)c2ccc(-c3cc(-c4ccccc4)cc(-c4ccccc4)c3)cc2)c1[2H] ; [2H]c1c([2H])c(-c2ccc3ccccc3c2)c([2H])c(N(c2ccc(-c3ccc(-c4ccccc4)cc3)cc2)c2ccc(-c3cc(-c4ccccc4)cc(-c4ccccc4)c3)cc2)c1[2H]]

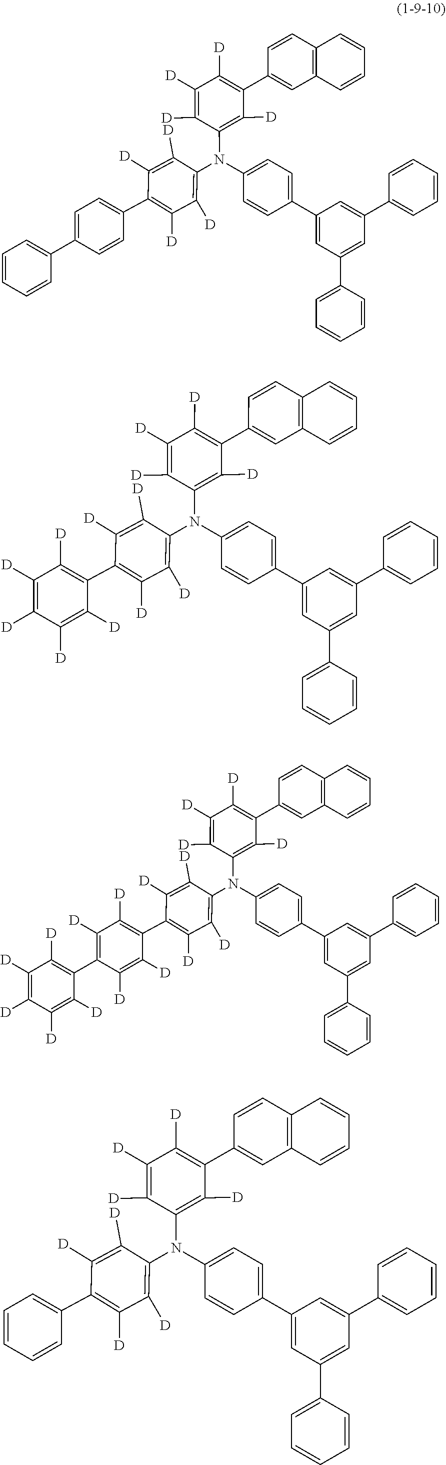

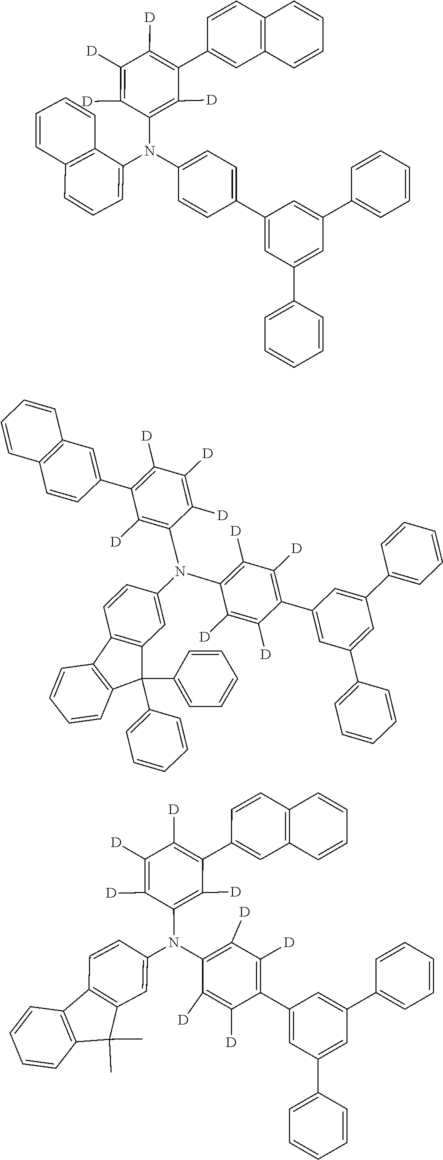

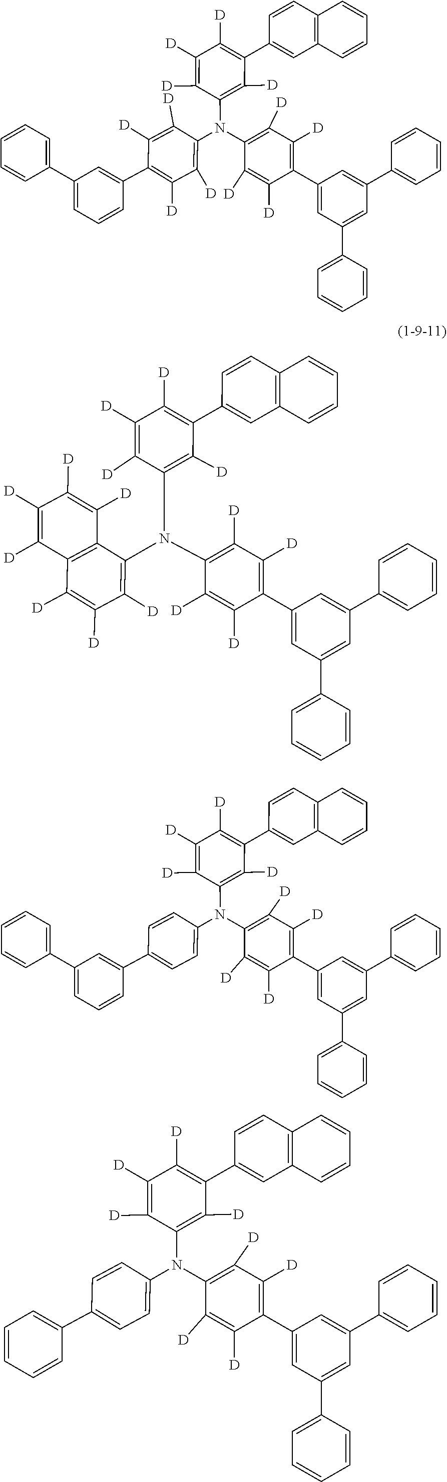

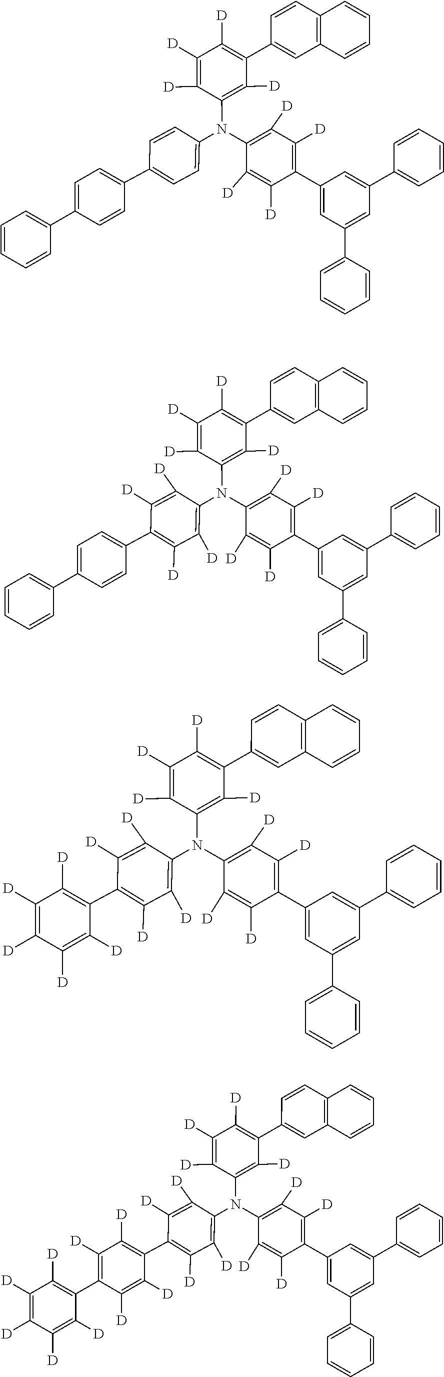

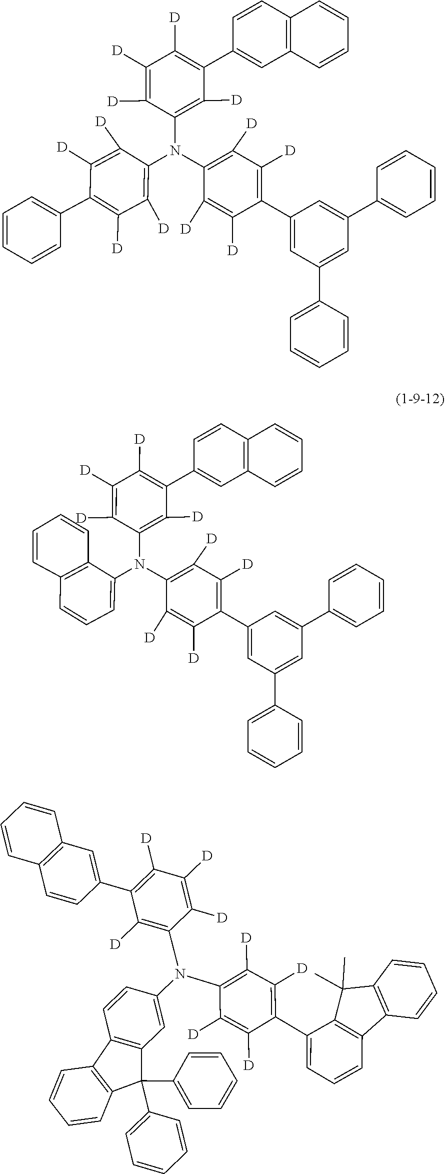 [SMILES: [2H]c1c([2H])c(-c2ccc3ccccc3c2)c([2H])c(N(c2c([2H])c([2H])c(-c3ccccc3)c([2H])c2[2H])c2c([2H])c([2H])c(-c3cc(-c4ccccc4)cc(-c4ccccc4)c3)c([2H])c2[2H])c1[2H] ; [2H]c1c([2H])c(-c2ccc3ccccc3c2)c([2H])c(N(c2c([2H])c([2H])c(-c3cc(-c4ccccc4)cc(-c4ccccc4)c3)c([2H])c2[2H])c2cccc3ccccc23)c1[2H] ; [2H]c1c([2H])c(-c2ccc3ccccc3c2)c([2H])c(N(c2ccc3c(c2)C(c2ccccc2)(c2ccccc2)c2ccccc2-3)c2c([2H])c([2H])c(-c3cccc4c3C(C)(C)c3ccccc3-4)c([2H])c2[2H])c1[2H]]

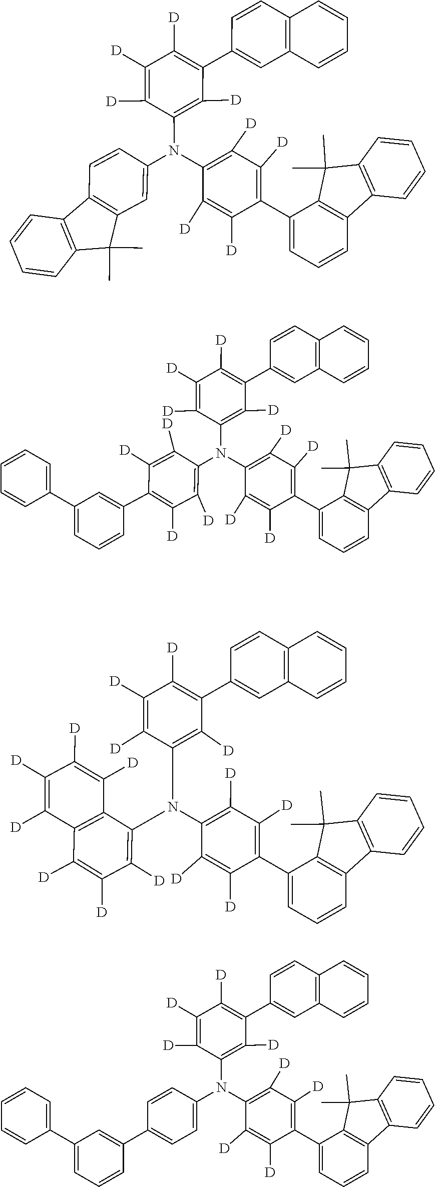

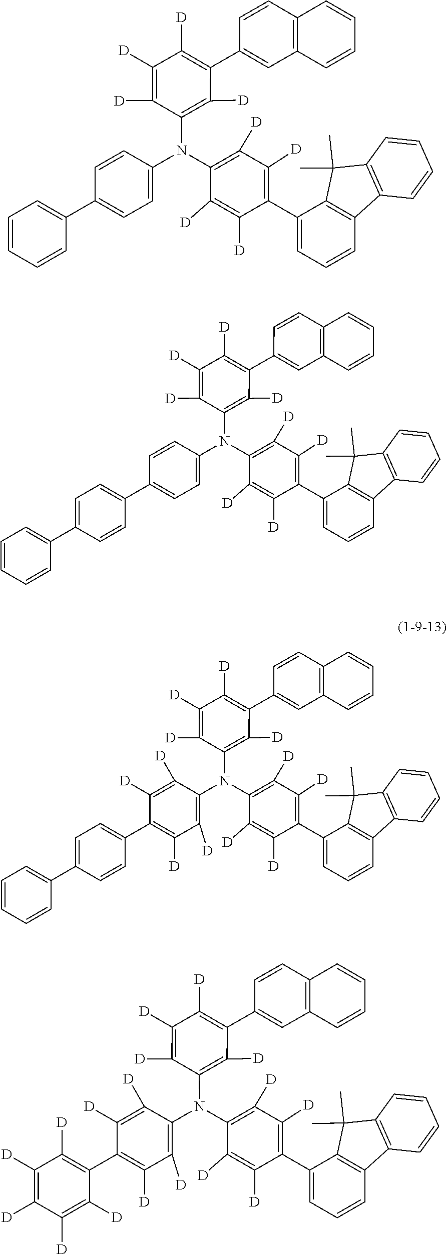

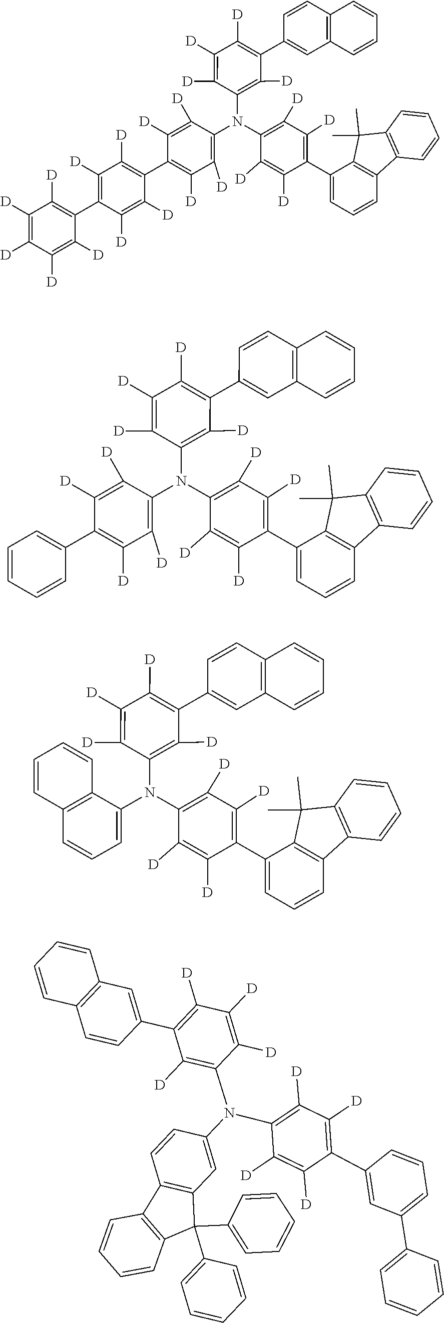

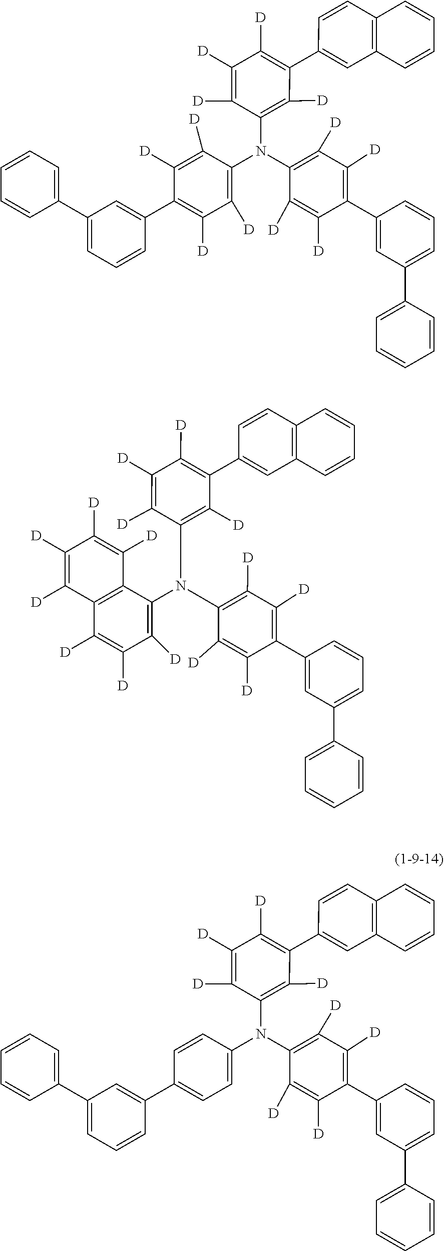 [SMILES: [2H]c1c([2H])c(-c2ccc3ccccc3c2)c([2H])c(N(c2c([2H])c([2H])c(-c3cccc(-c4ccccc4)c3)c([2H])c2[2H])c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2c([2H])c([2H])c(-c3cccc(-c4ccccc4)c3)c([2H])c2[2H])c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc(-c3cccc(-c4ccccc4)c3)cc2)c2c([2H])c([2H])c(-c3cccc(-c4ccccc4)c3)c([2H])c2[2H])c1[2H]]

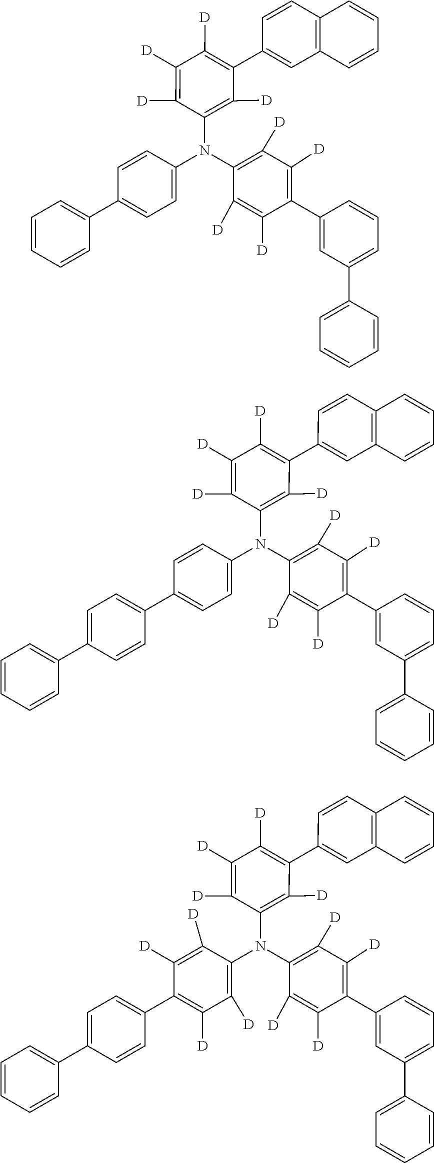

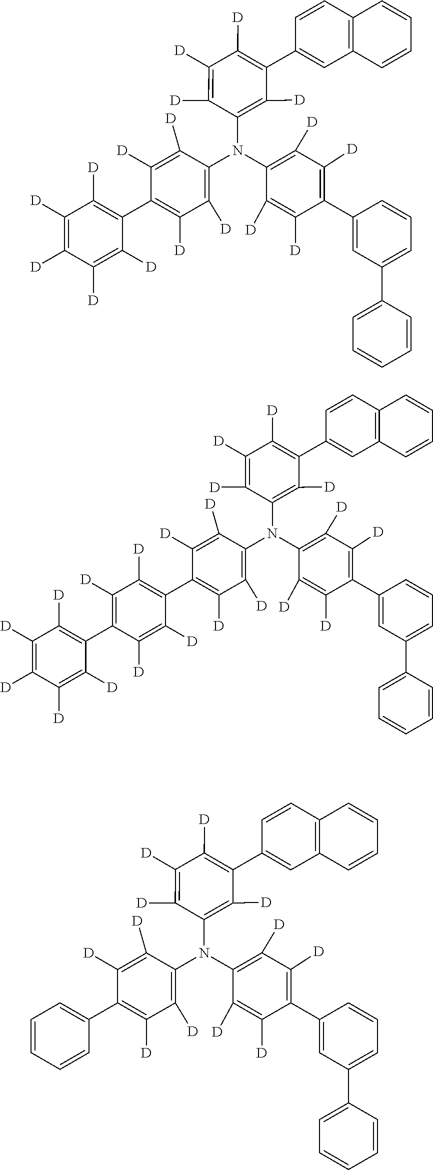

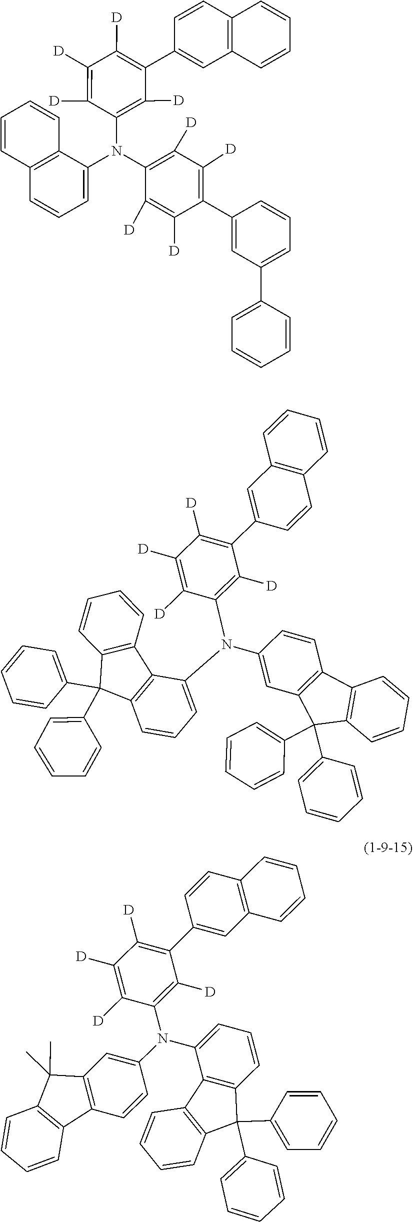 [SMILES: [2H]c1c([2H])c(-c2ccc3ccccc3c2)c([2H])c(N(c2c([2H])c([2H])c(-c3cccc(-c4ccccc4)c3)c([2H])c2[2H])c2cccc3ccccc23)c1[2H] ; [2H]c1c([2H])c(-c2ccc3ccccc3c2)c([2H])c(N(c2ccc3c(c2)C(c2ccccc2)(c2ccccc2)c2ccccc2-3)c2cccc3c2-c2ccccc2C3(c2ccccc2)c2ccccc2)c1[2H] ; [2H]c1c([2H])c(-c2ccc3ccccc3c2)c([2H])c(N(c2ccc3c(c2)C(C)(C)c2ccccc2-3)c2cccc3c2-c2ccccc2C3(c2ccccc2)c2ccccc2)c1[2H]]

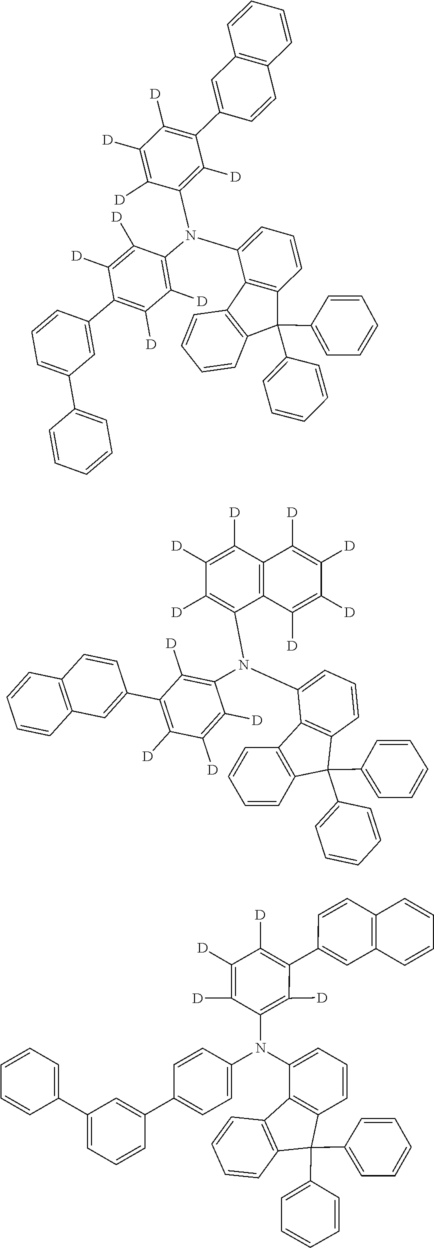

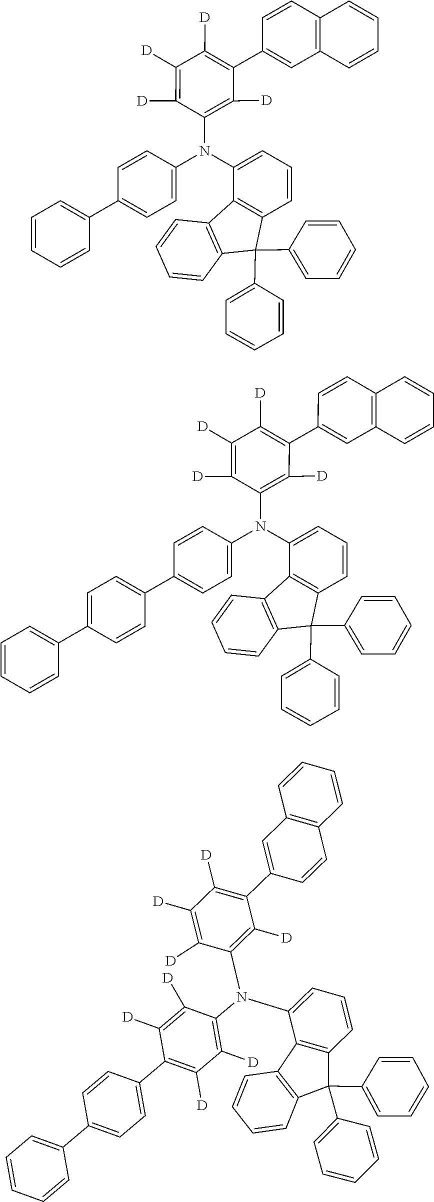

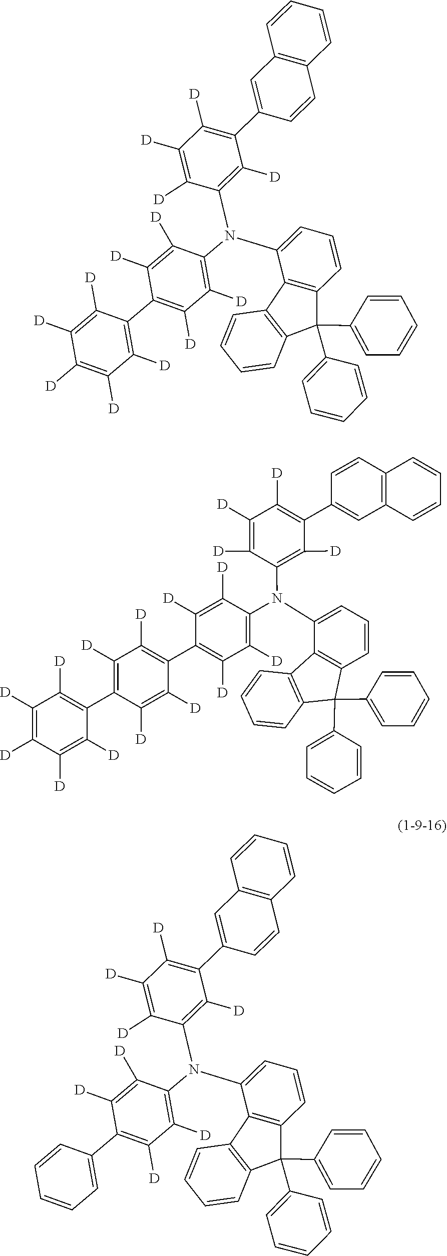

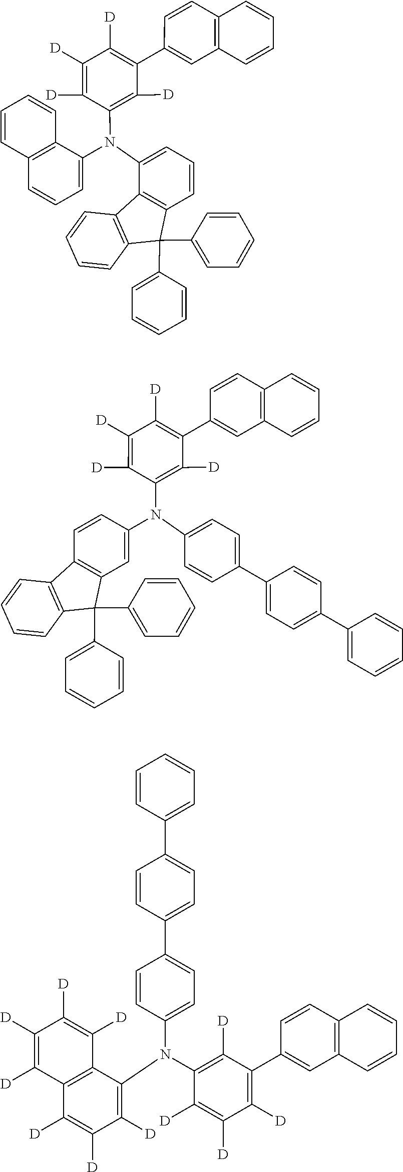 [SMILES: [2H]c1c([2H])c(-c2ccc3ccccc3c2)c([2H])c(N(c2cccc3c2-c2ccccc2C3(c2ccccc2)c2ccccc2)c2cccc3ccccc23)c1[2H] ; [2H]c1c([2H])c(-c2ccc3ccccc3c2)c([2H])c(N(c2ccc(-c3ccc(-c4ccccc4)cc3)cc2)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccc(-c3ccc(-c4ccccc4)cc3)cc2)c2c([2H])c([2H])c([2H])c3c([2H])c([2H])c([2H])c([2H])c23)c1[2H]]

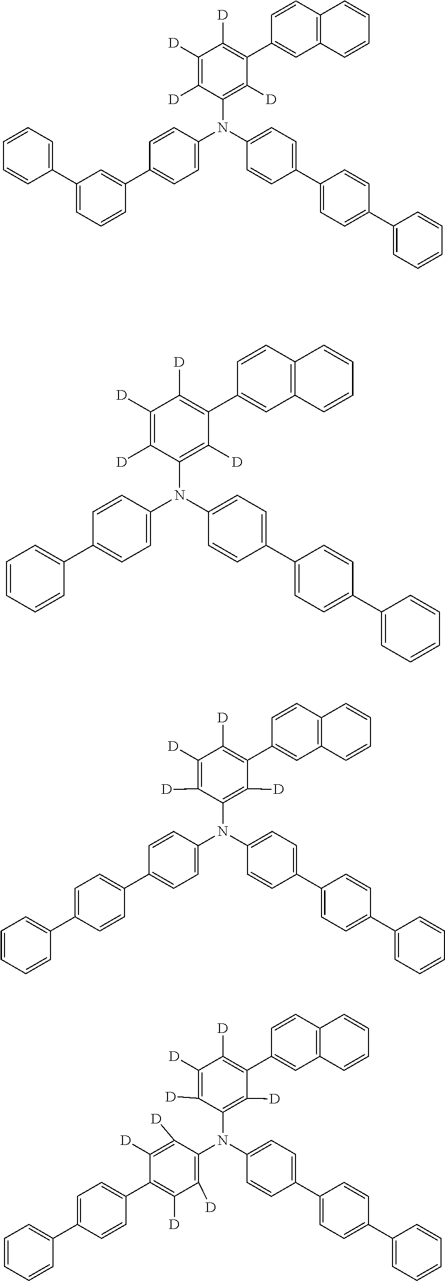 [SMILES: [2H]c1c([2H])c(-c2ccc3ccccc3c2)c([2H])c(N(c2ccc(-c3ccc(-c4ccccc4)cc3)cc2)c2ccc(-c3cccc(-c4ccccc4)c3)cc2)c1[2H] ; [2H]c1c([2H])c(-c2ccc3ccccc3c2)c([2H])c(N(c2ccc(-c3ccccc3)cc2)c2ccc(-c3ccc(-c4ccccc4)cc3)cc2)c1[2H] ; [2H]c1c([2H])c(-c2ccc3ccccc3c2)c([2H])c(N(c2ccc(-c3ccc(-c4ccccc4)cc3)cc2)c2ccc(-c3ccc(-c4ccccc4)cc3)cc2)c1[2H] ; [2H]c1c([2H])c(-c2ccc3ccccc3c2)c([2H])c(N(c2ccc(-c3ccc(-c4ccccc4)cc3)cc2)c2c([2H])c([2H])c(-c3ccc(-c4ccccc4)cc3)c([2H])c2[2H])c1[2H]]

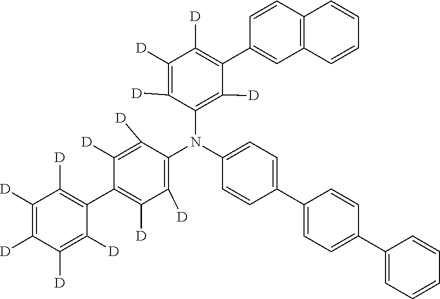 [SMILES: [2H]c1c([2H])c([2H])c(-c2c([2H])c([2H])c(N(c3ccc(-c4ccc(-c5ccccc5)cc4)cc3)c3c([2H])c([2H])c([2H])c(-c4ccc5ccccc5c4)c3[2H])c([2H])c2[2H])c([2H])c1[2H]]

(1-9-17)

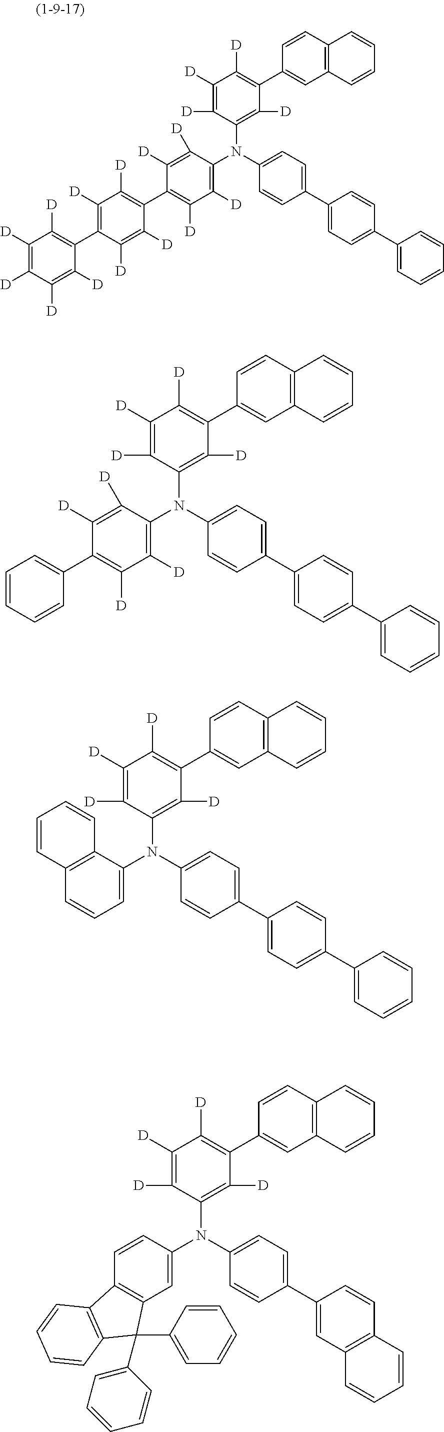

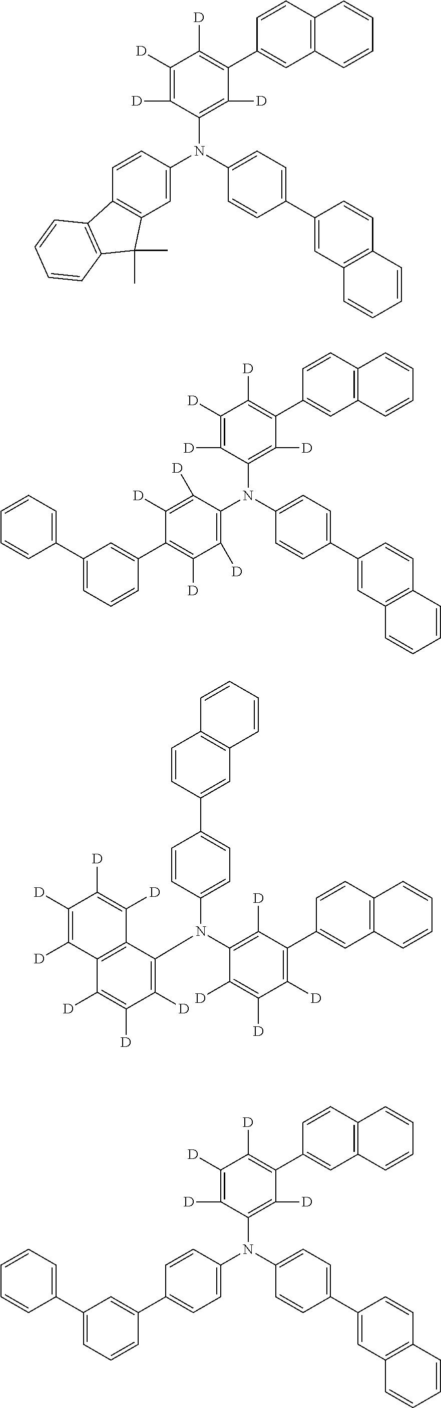

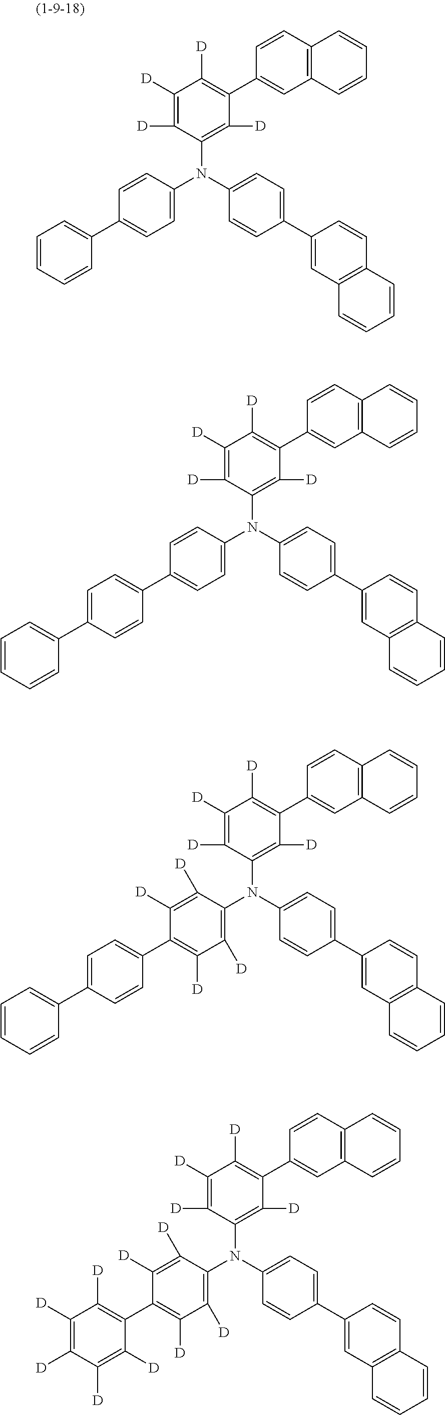 [SMILES: [2H]c1c([2H])c(-c2ccc3ccccc3c2)c([2H])c(N(c2ccc(-c3ccccc3)cc2)c2ccc(-c3ccc4ccccc4c3)cc2)c1[2H] ; [2H]c1c([2H])c(-c2ccc3ccccc3c2)c([2H])c(N(c2ccc(-c3ccc(-c4ccccc4)cc3)cc2)c2ccc(-c3ccc4ccccc4c3)cc2)c1[2H] ; [2H]c1c([2H])c(-c2ccc3ccccc3c2)c([2H])c(N(c2ccc(-c3ccc4ccccc4c3)cc2)c2c([2H])c([2H])c(-c3ccc(-c4ccccc4)cc3)c([2H])c2[2H])c1[2H] ; [2H]c1c([2H])c([2H])c(-c2c([2H])c([2H])c(N(c3ccc(-c4ccc5ccccc5c4)cc3)c3c([2H])c([2H])c([2H])c(-c4ccc5ccccc5c4)c3[2H])c([2H])c2[2H])c([2H])c1[2H]]

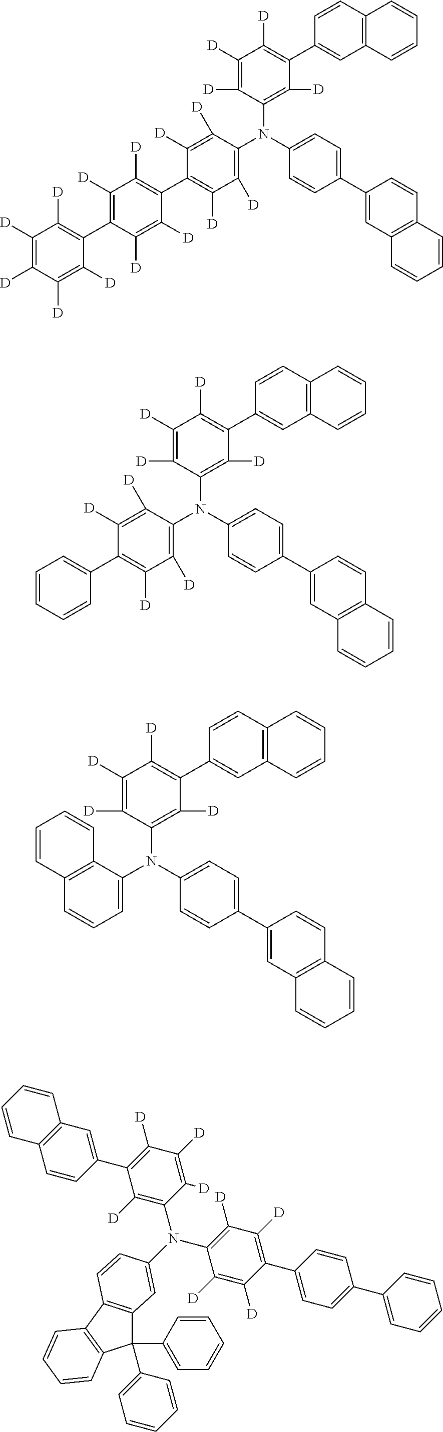

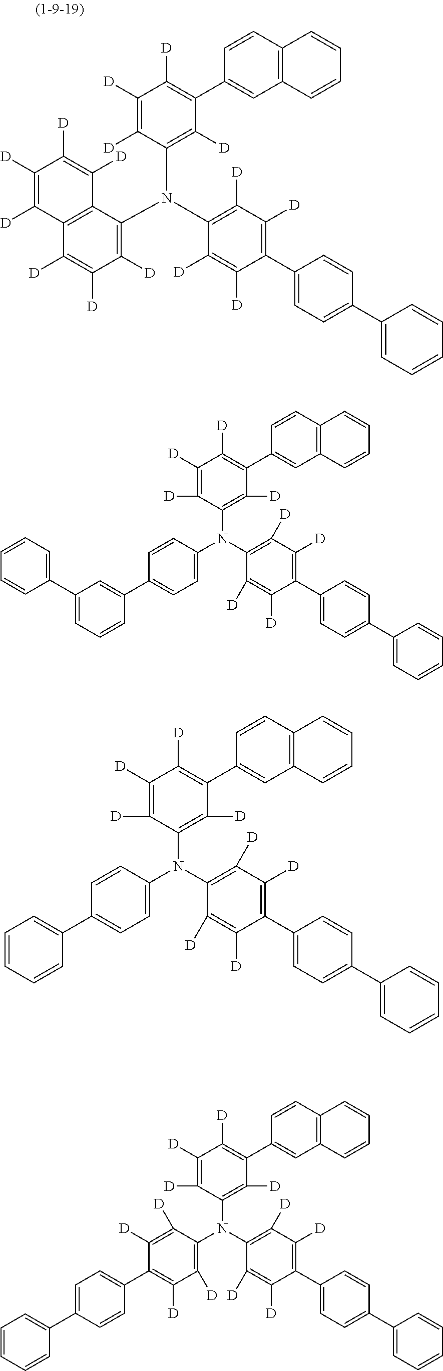

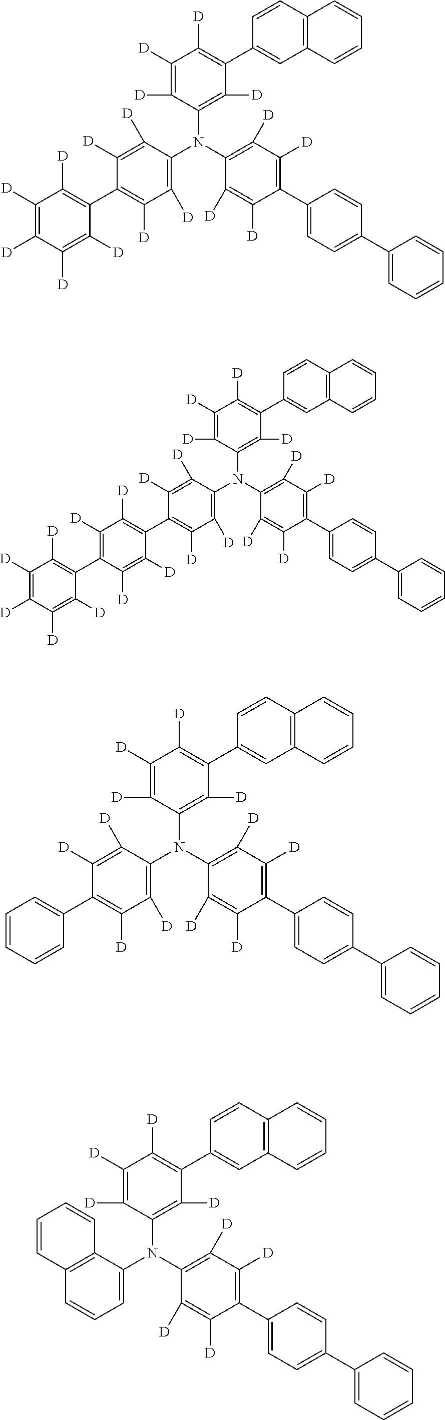

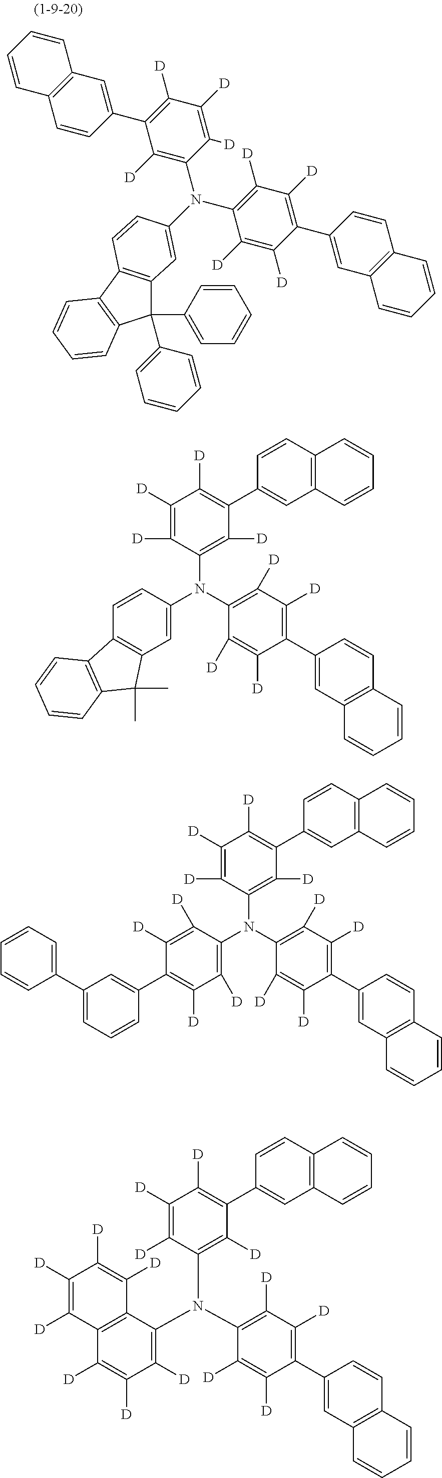

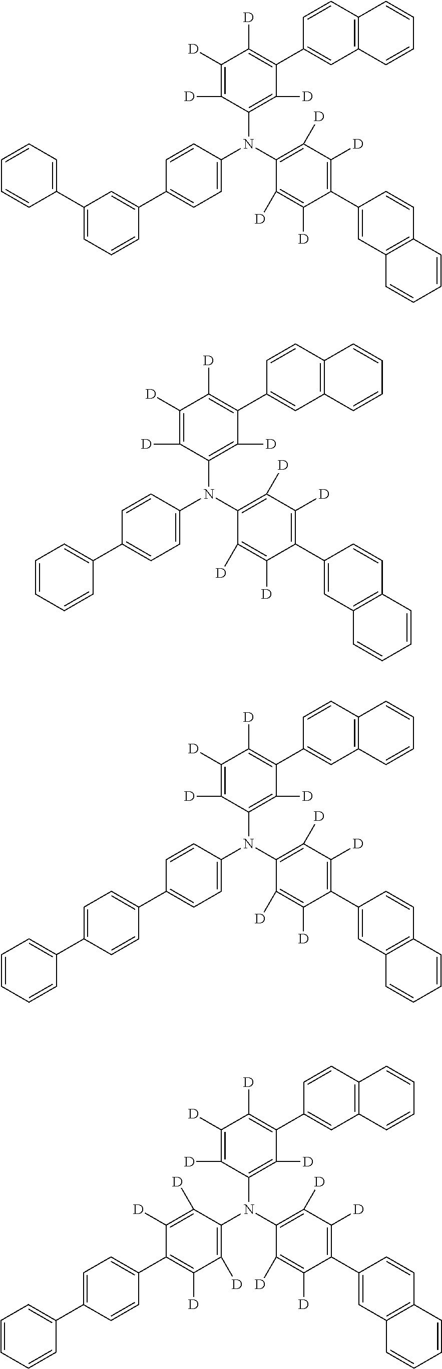

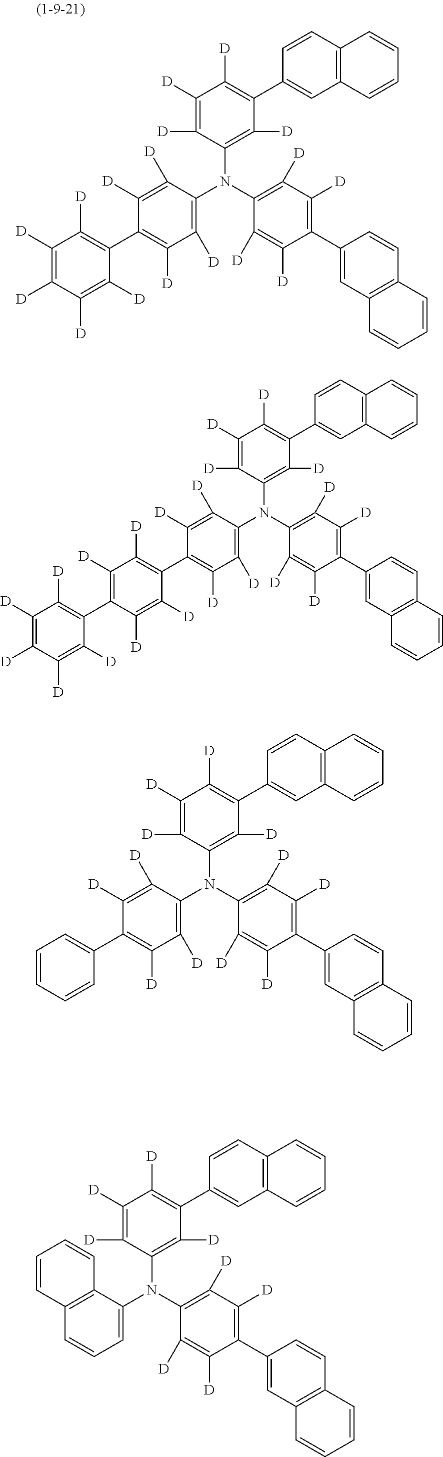

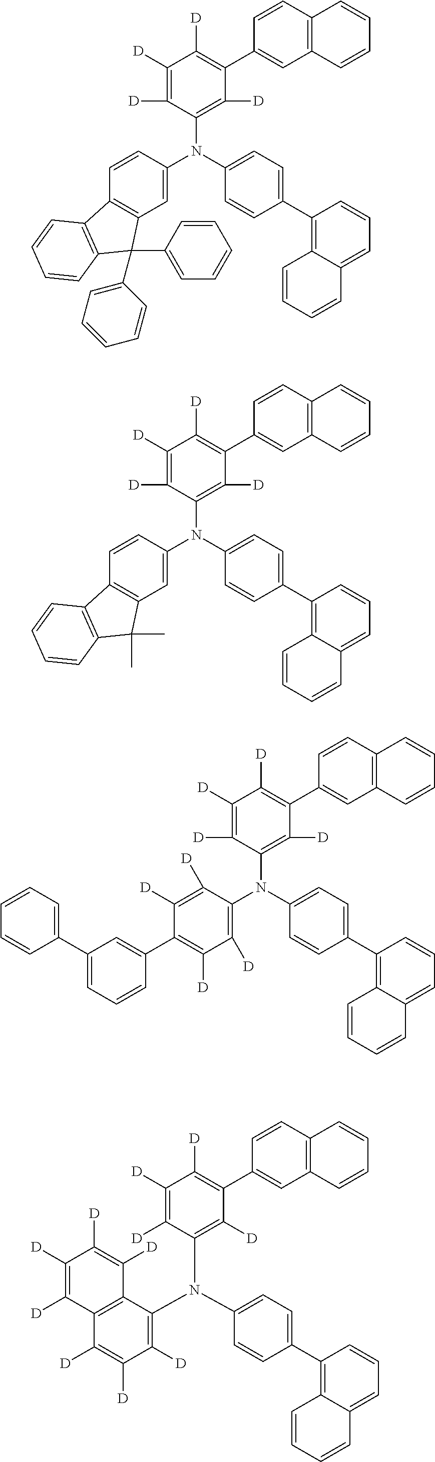 [SMILES: [2H]c1c([2H])c(-c2ccc3ccccc3c2)c([2H])c(N(c2ccc(-c3cccc4ccccc34)cc2)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccc(-c3cccc4ccccc34)cc2)c2ccc3c(c2)C(C)(C)c2ccccc2-3)c1[2H] ; [2H]c1c([2H])c(-c2ccc3ccccc3c2)c([2H])c(N(c2ccc(-c3cccc4ccccc34)cc2)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2ccc(-c3cccc4ccccc34)cc2)c2c([2H])c([2H])c([2H])c3c([2H])c([2H])c([2H])c([2H])c23)c1[2H]]

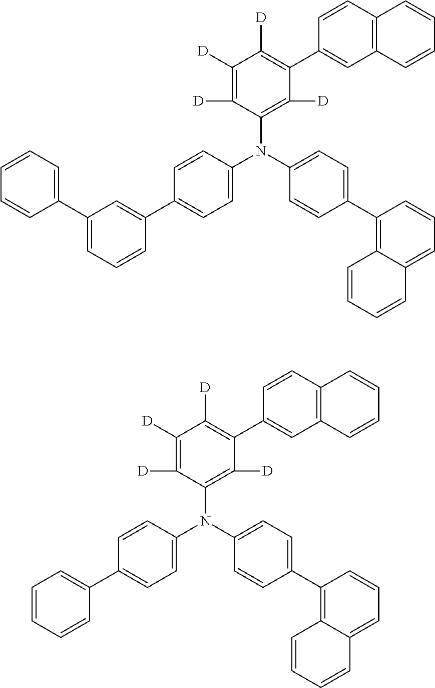 [SMILES: [2H]c1c([2H])c(-c2ccc3ccccc3c2)c([2H])c(N(c2ccc(-c3cccc(-c4ccccc4)c3)cc2)c2ccc(-c3cccc4ccccc34)cc2)c1[2H] ; [2H]c1c([2H])c(-c2ccc3ccccc3c2)c([2H])c(N(c2ccc(-c3ccccc3)cc2)c2ccc(-c3cccc4ccccc34)cc2)c1[2H]]

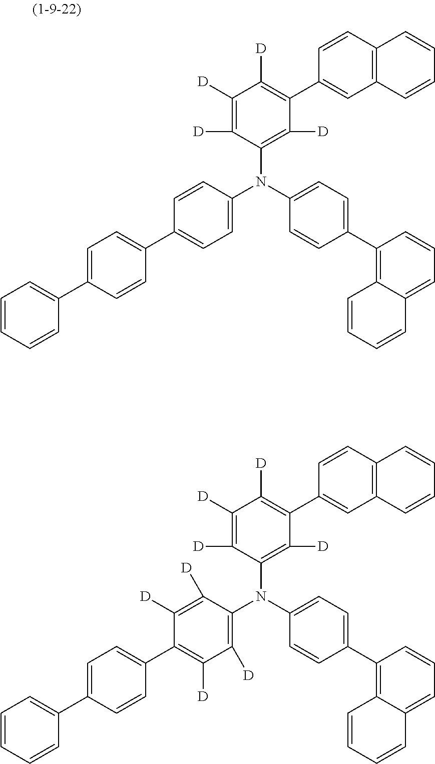 [SMILES: [2H]c1c([2H])c(-c2ccc3ccccc3c2)c([2H])c(N(c2ccc(-c3ccc(-c4ccccc4)cc3)cc2)c2ccc(-c3cccc4ccccc34)cc2)c1[2H] ; [2H]c1c([2H])c(-c2ccc3ccccc3c2)c([2H])c(N(c2ccc(-c3cccc4ccccc34)cc2)c2c([2H])c([2H])c(-c3ccc(-c4ccccc4)cc3)c([2H])c2[2H])c1[2H]]

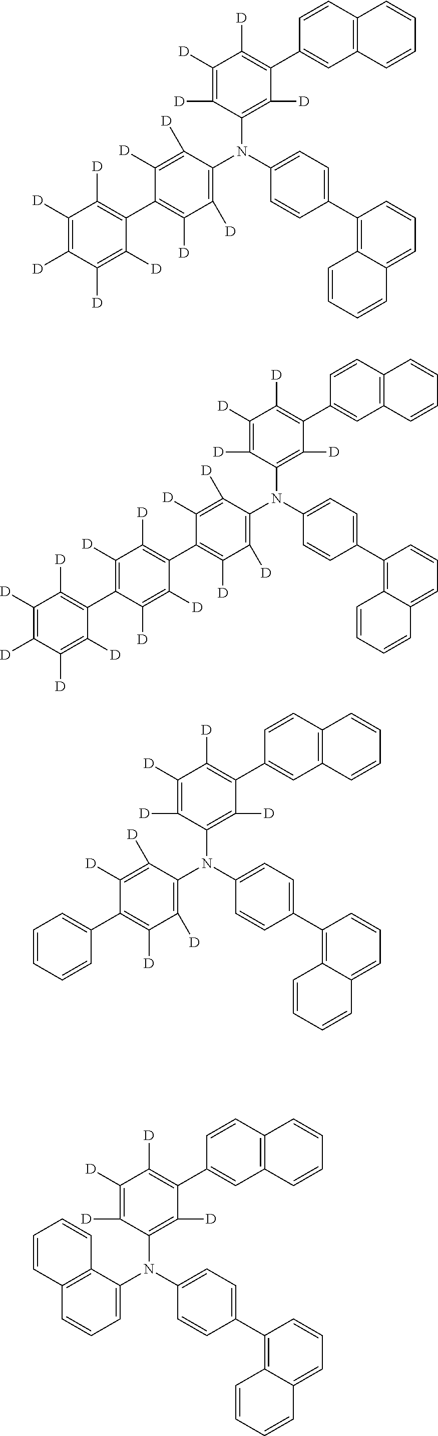

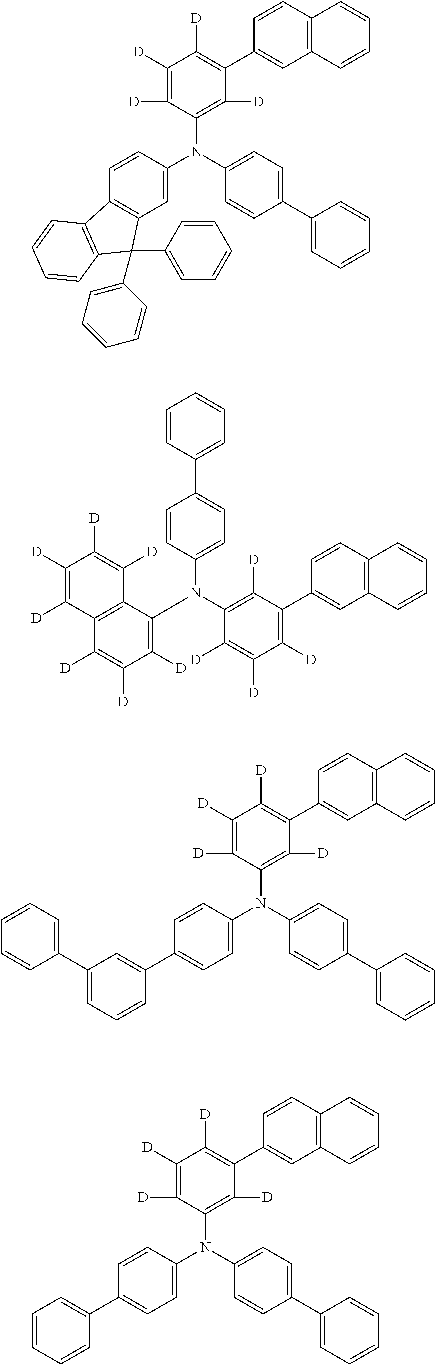 [SMILES: [2H]c1c([2H])c(-c2ccc3ccccc3c2)c([2H])c(N(c2ccc(-c3ccccc3)cc2)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccc(-c3ccccc3)cc2)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc(-c3ccccc3)cc2)c2ccc(-c3cccc(-c4ccccc4)c3)cc2)c1[2H] ; [2H]c1c([2H])c(-c2ccc3ccccc3c2)c([2H])c(N(c2ccc(-c3ccccc3)cc2)c2ccc(-c3ccccc3)cc2)c1[2H]]

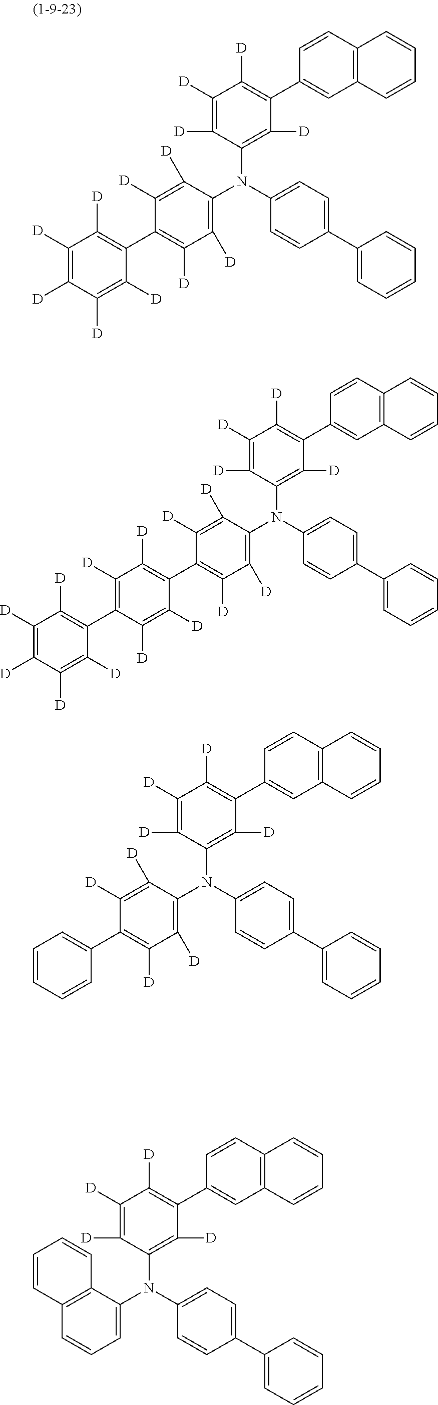

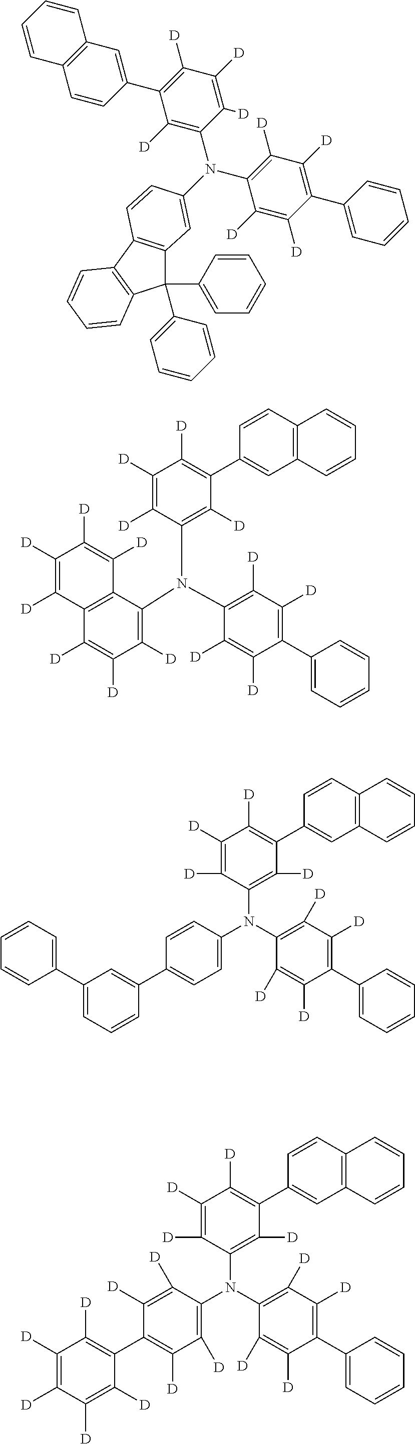

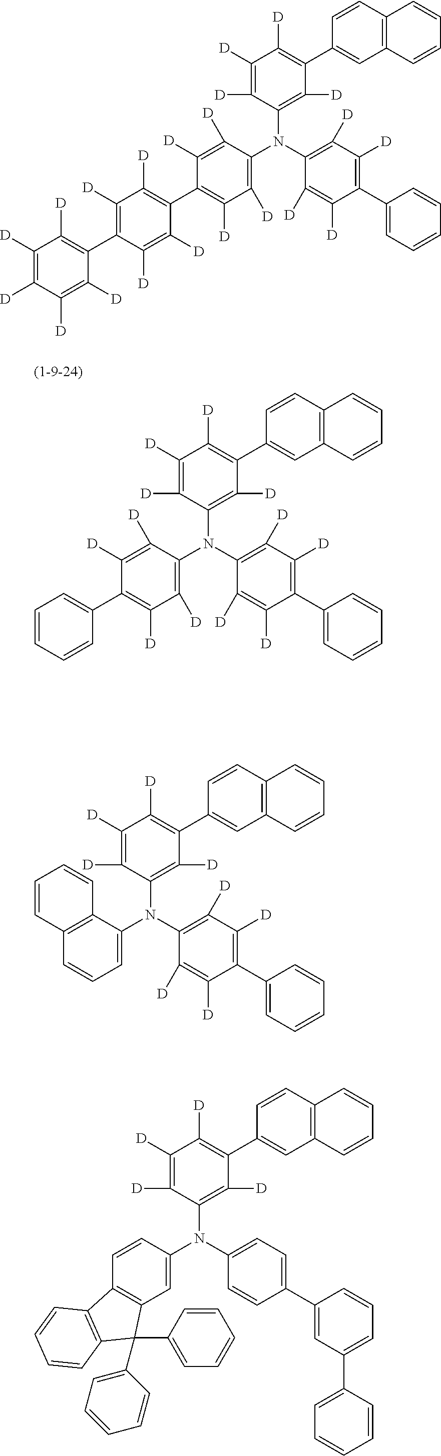

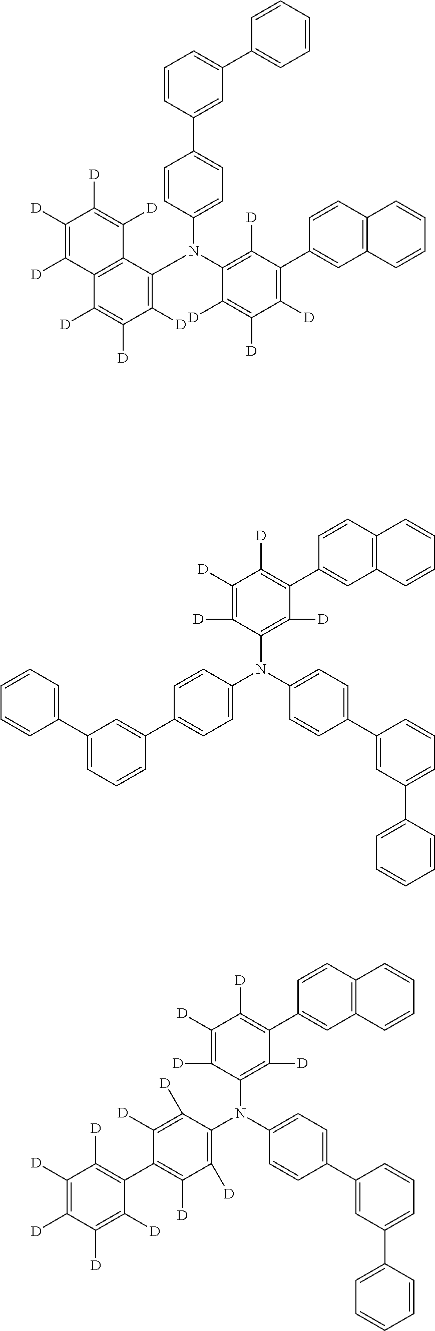 [SMILES: [2H]c1c([2H])c(-c2ccc3ccccc3c2)c([2H])c(N(c2ccc(-c3cccc(-c4ccccc4)c3)cc2)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc(-c3cccc(-c4ccccc4)c3)cc2)c2ccc(-c3cccc(-c4ccccc4)c3)cc2)c1[2H] ; [2H]c1c([2H])c([2H])c(-c2c([2H])c([2H])c(N(c3ccc(-c4cccc(-c5ccccc5)c4)cc3)c3c([2H])c([2H])c([2H])c(-c4ccc5ccccc5c4)c3[2H])c([2H])c2[2H])c([2H])c1[2H]]

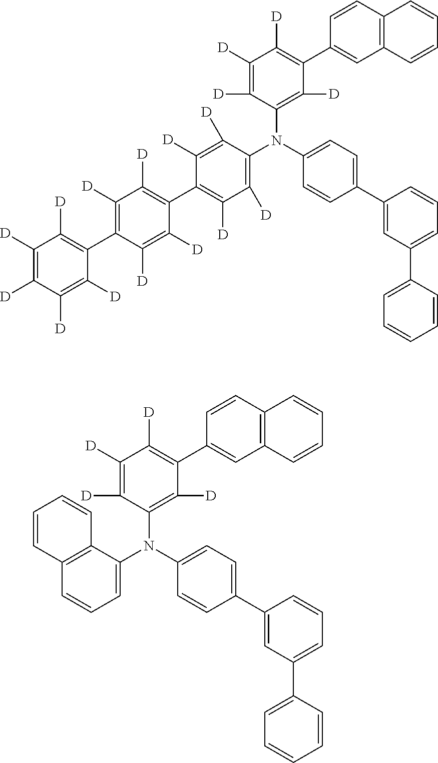 [SMILES: [2H]c1c([2H])c([2H])c(-c2c([2H])c([2H])c(-c3c([2H])c([2H])c(N(c4ccc(-c5cccc(-c6ccccc6)c5)cc4)c4c([2H])c([2H])c([2H])c(-c5ccc6ccccc6c5)c4[2H])c([2H])c3[2H])c([2H])c2[2H])c([2H])c1[2H] ; [2H]c1c([2H])c(-c2ccc3ccccc3c2)c([2H])c(N(c2ccc(-c3cccc(-c4ccccc4)c3)cc2)c2cccc3ccccc23)c1[2H]]

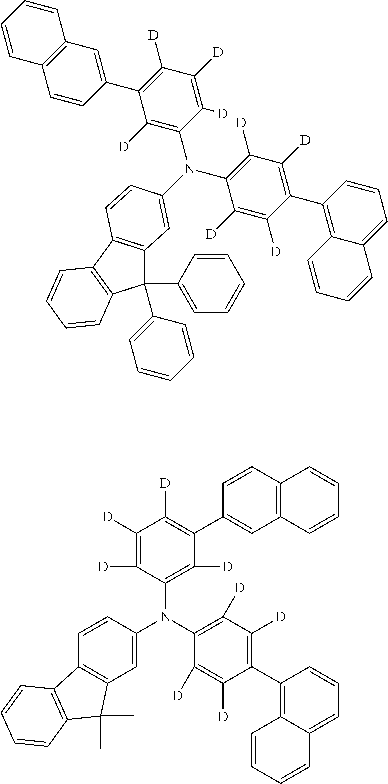 [SMILES: [2H]c1c([2H])c(-c2ccc3ccccc3c2)c([2H])c(N(c2ccc3c(c2)C(c2ccccc2)(c2ccccc2)c2ccccc2-3)c2c([2H])c([2H])c(-c3cccc4ccccc34)c([2H])c2[2H])c1[2H] ; [2H]c1c([2H])c(-c2ccc3ccccc3c2)c([2H])c(N(c2ccc3c(c2)C(C)(C)c2ccccc2-3)c2c([2H])c([2H])c(-c3cccc4ccccc34)c([2H])c2[2H])c1[2H]]

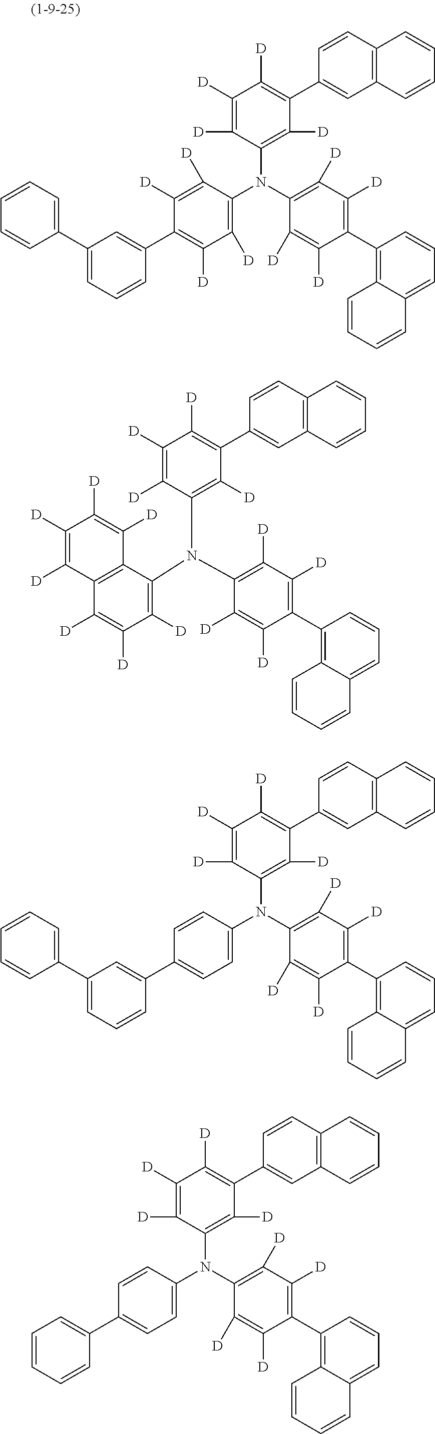

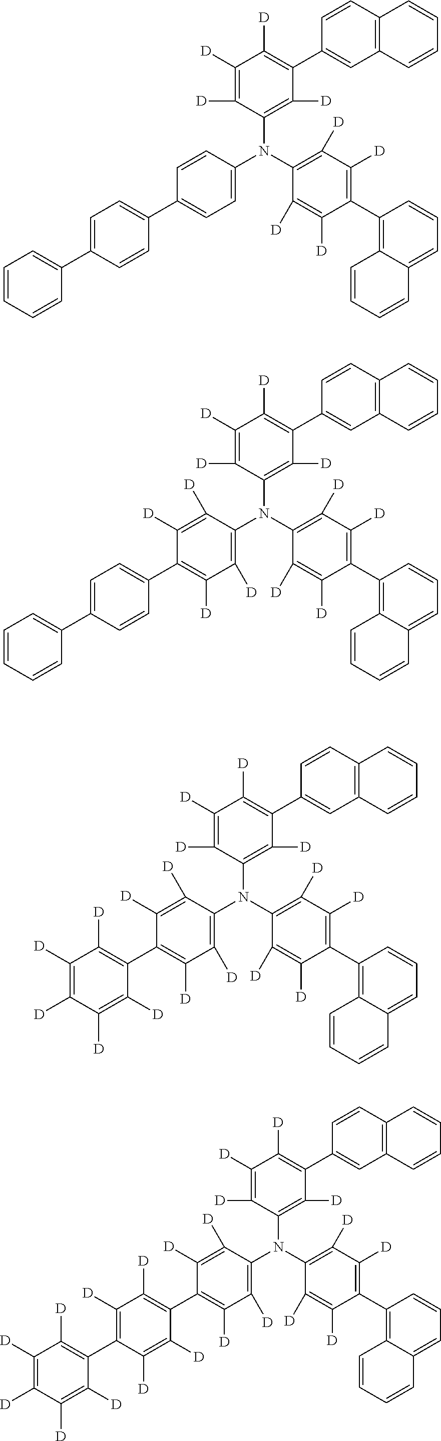

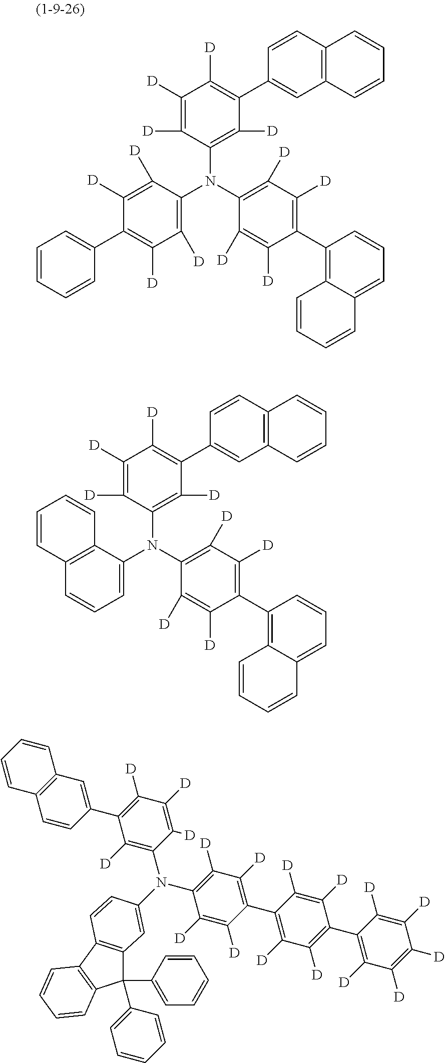 [SMILES: [2H]c1c([2H])c(-c2ccc3ccccc3c2)c([2H])c(N(c2c([2H])c([2H])c(-c3ccccc3)c([2H])c2[2H])c2c([2H])c([2H])c(-c3cccc4ccccc34)c([2H])c2[2H])c1[2H] ; [2H]c1c([2H])c(-c2ccc3ccccc3c2)c([2H])c(N(c2c([2H])c([2H])c(-c3cccc4ccccc34)c([2H])c2[2H])c2cccc3ccccc23)c1[2H] ; [2H]c1c([2H])c([2H])c(-c2c([2H])c([2H])c(-c3c([2H])c([2H])c(N(c4ccc5c(c4)C(c4ccccc4)(c4ccccc4)c4ccccc4-5)c4c([2H])c([2H])c([2H])c(-c5ccc6ccccc6c5)c4[2H])c([2H])c3[2H])c([2H])c2[2H])c([2H])c1[2H]]

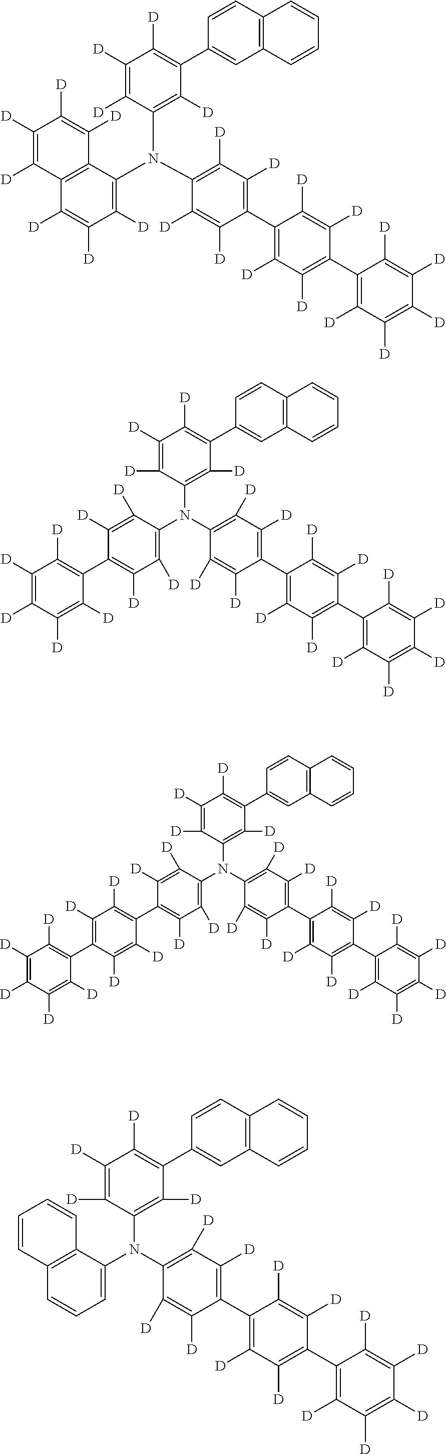

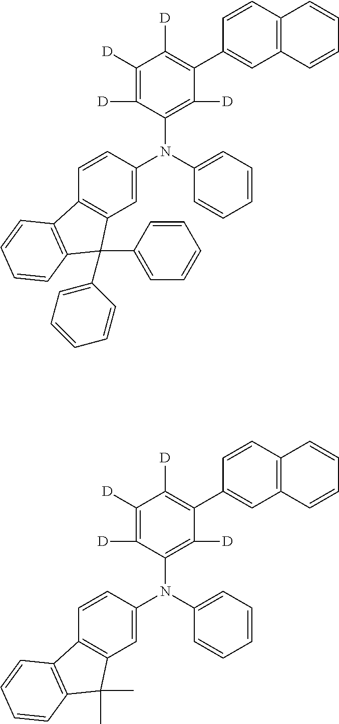 [SMILES: [2H]c1c([2H])c(-c2ccc3ccccc3c2)c([2H])c(N(c2ccccc2)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccccc2)c2ccc3c(c2)C(C)(C)c2ccccc2-3)c1[2H]]

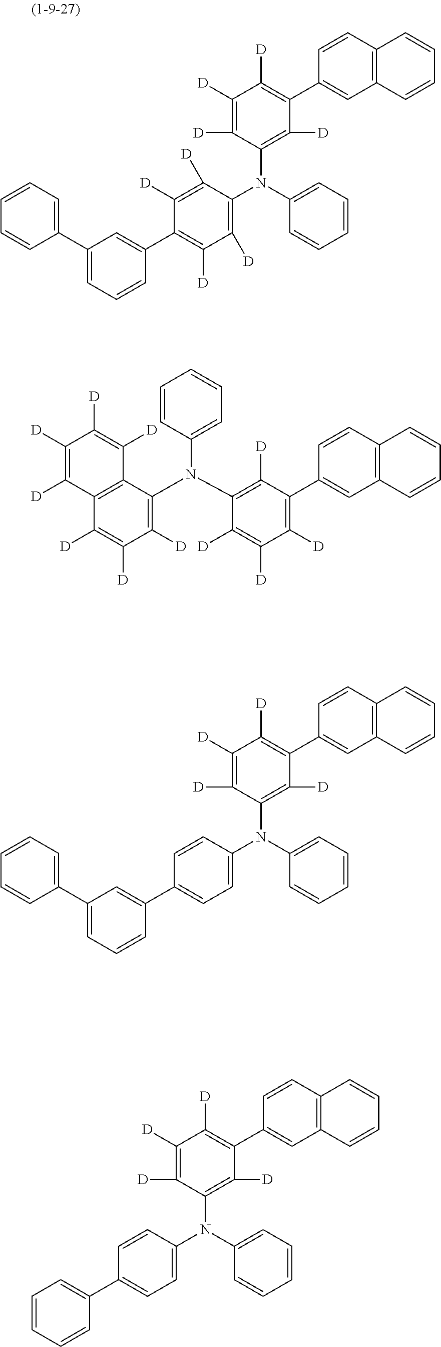 [SMILES: [2H]c1c([2H])c(-c2ccc3ccccc3c2)c([2H])c(N(c2ccccc2)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2ccccc2)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccccc2)c2ccc(-c3cccc(-c4ccccc4)c3)cc2)c1[2H] ; [2H]c1c([2H])c(-c2ccc3ccccc3c2)c([2H])c(N(c2ccccc2)c2ccc(-c3ccccc3)cc2)c1[2H]]

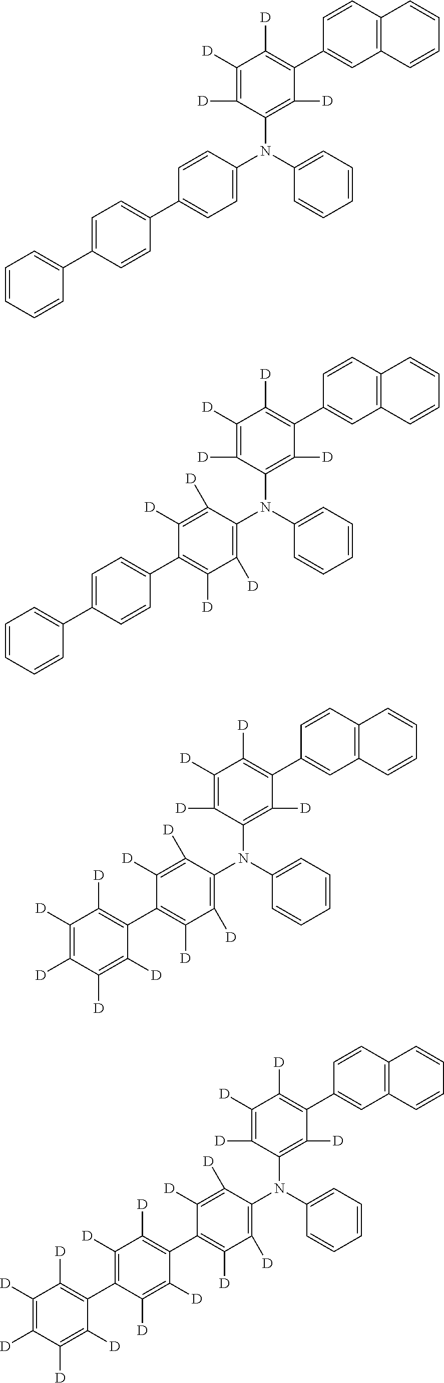 [SMILES: [2H]c1c([2H])c(-c2ccc3ccccc3c2)c([2H])c(N(c2ccccc2)c2ccc(-c3ccc(-c4ccccc4)cc3)cc2)c1[2H] ; [2H]c1c([2H])c(-c2ccc3ccccc3c2)c([2H])c(N(c2ccccc2)c2c([2H])c([2H])c(-c3ccc(-c4ccccc4)cc3)c([2H])c2[2H])c1[2H] ; [2H]c1c([2H])c([2H])c(-c2c([2H])c([2H])c(N(c3ccccc3)c3c([2H])c([2H])c([2H])c(-c4ccc5ccccc5c4)c3[2H])c([2H])c2[2H])c([2H])c1[2H] ; [2H]c1c([2H])c([2H])c(-c2c([2H])c([2H])c(-c3c([2H])c([2H])c(N(c4ccccc4)c4c([2H])c([2H])c([2H])c(-c5ccc6ccccc6c5)c4[2H])c([2H])c3[2H])c([2H])c2[2H])c([2H])c1[2H]]

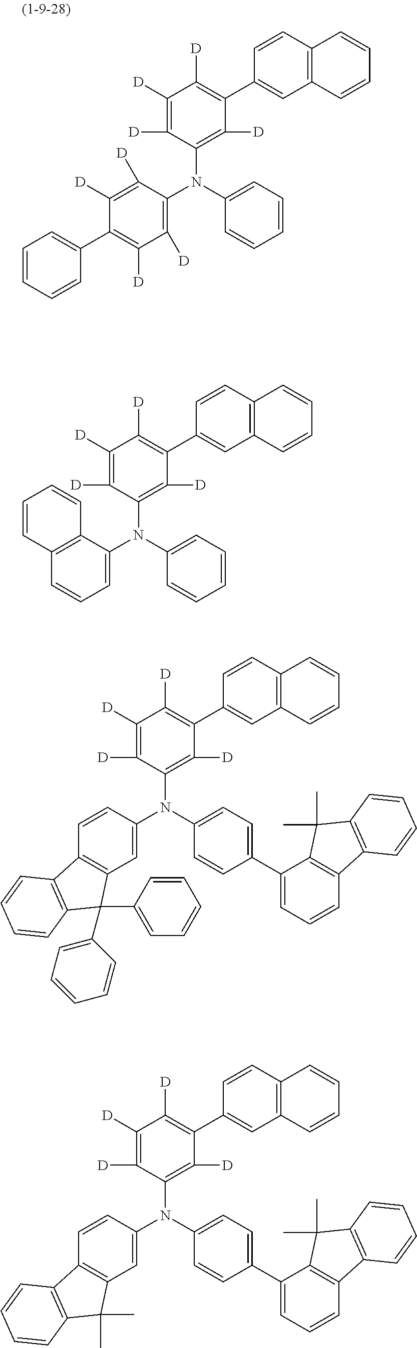 [SMILES: [2H]c1c([2H])c(-c2ccc3ccccc3c2)c([2H])c(N(c2ccccc2)c2c([2H])c([2H])c(-c3ccccc3)c([2H])c2[2H])c1[2H] ; [2H]c1c([2H])c(-c2ccc3ccccc3c2)c([2H])c(N(c2ccccc2)c2cccc3ccccc23)c1[2H] ; [2H]c1c([2H])c(-c2ccc3ccccc3c2)c([2H])c(N(c2ccc(-c3cccc4c3C(C)(C)c3ccccc3-4)cc2)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccc(-c3cccc4c3C(C)(C)c3ccccc3-4)cc2)c2ccc3c(c2)C(C)(C)c2ccccc2-3)c1[2H]]

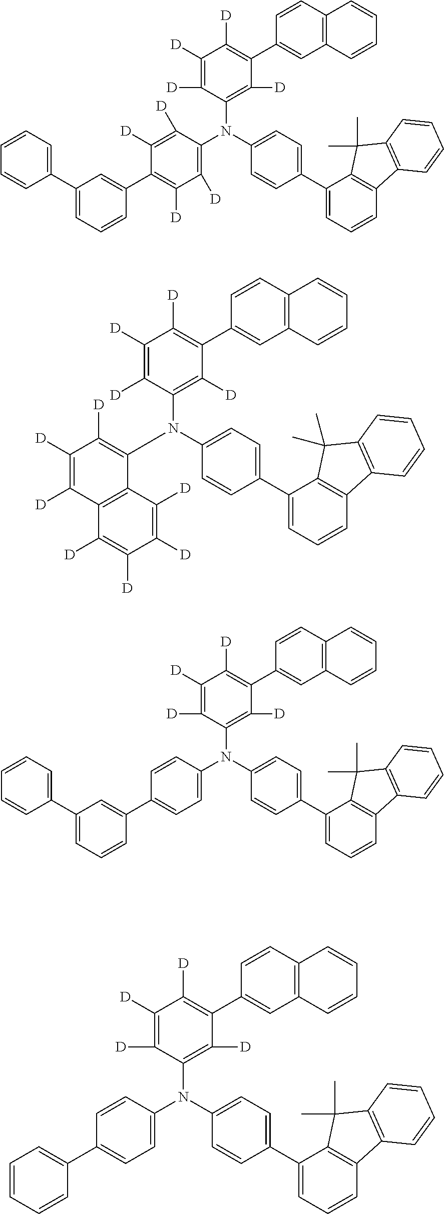

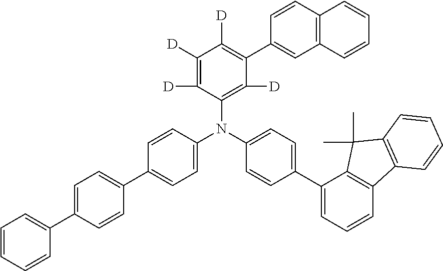 [SMILES: [2H]c1c([2H])c(-c2ccc3ccccc3c2)c([2H])c(N(c2ccc(-c3ccc(-c4ccccc4)cc3)cc2)c2ccc(-c3cccc4c3C(C)(C)c3ccccc3-4)cc2)c1[2H]]

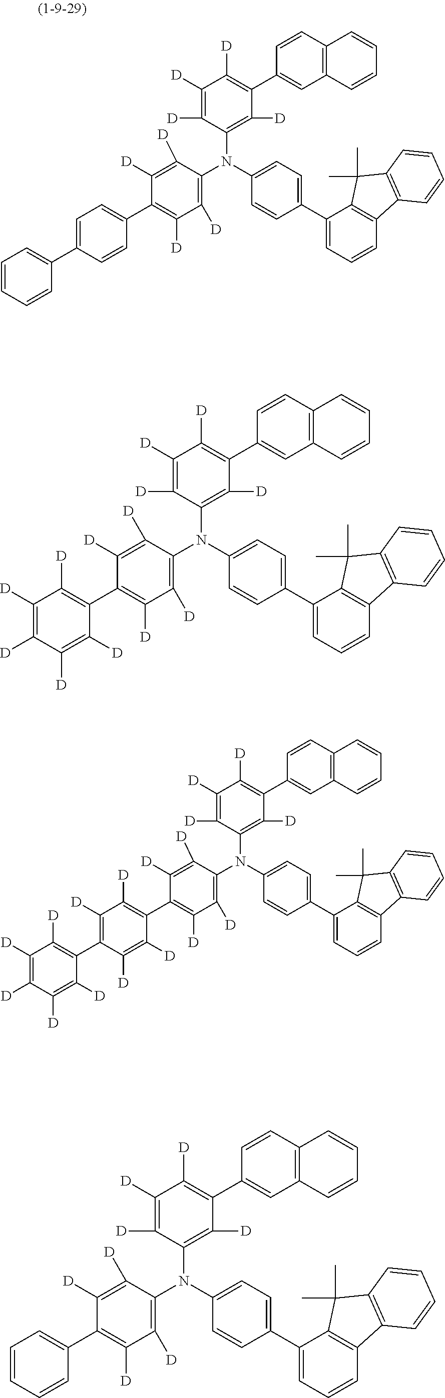

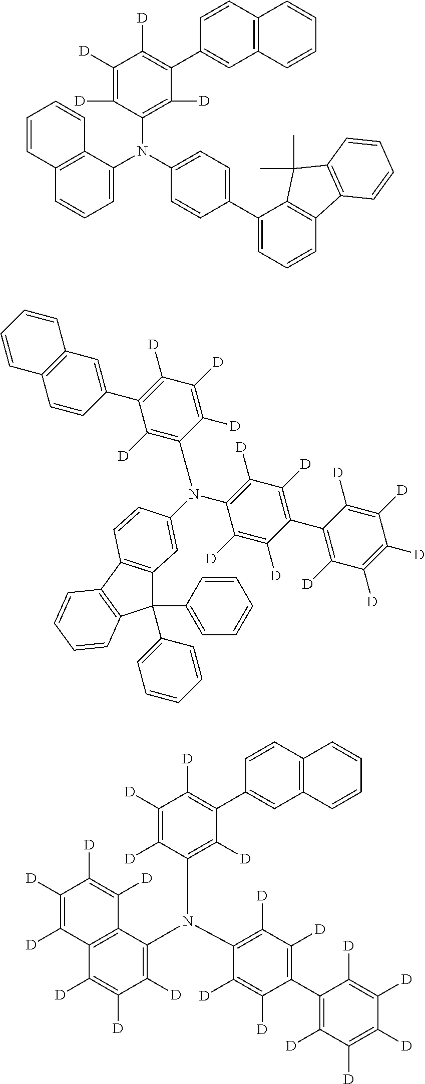 [SMILES: [2H]c1c([2H])c(-c2ccc3ccccc3c2)c([2H])c(N(c2ccc(-c3cccc4c3C(C)(C)c3ccccc3-4)cc2)c2cccc3ccccc23)c1[2H] ; [2H]c1c([2H])c([2H])c(-c2c([2H])c([2H])c(N(c3ccc4c(c3)C(c3ccccc3)(c3ccccc3)c3ccccc3-4)c3c([2H])c([2H])c([2H])c(-c4ccc5ccccc5c4)c3[2H])c([2H])c2[2H])c([2H])c1[2H] ; [2H]c1c([2H])c([2H])c(-c2c([2H])c([2H])c(N(c3c([2H])c([2H])c([2H])c(-c4ccc5ccccc5c4)c3[2H])c3c([2H])c([2H])c([2H])c4c([2H])c([2H])c([2H])c([2H])c34)c([2H])c2[2H])c([2H])c1[2H]]

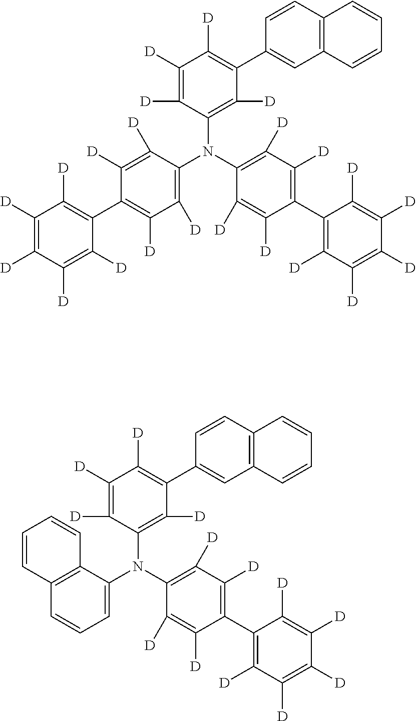 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c([2H])c(-c4ccc5ccccc5c4)c3[2H])c([2H])c2[2H])c([2H])c1[2H] ; [2H]c1c([2H])c([2H])c(-c2c([2H])c([2H])c(N(c3c([2H])c([2H])c([2H])c(-c4ccc5ccccc5c4)c3[2H])c3cccc4ccccc34)c([2H])c2[2H])c([2H])c1[2H]]

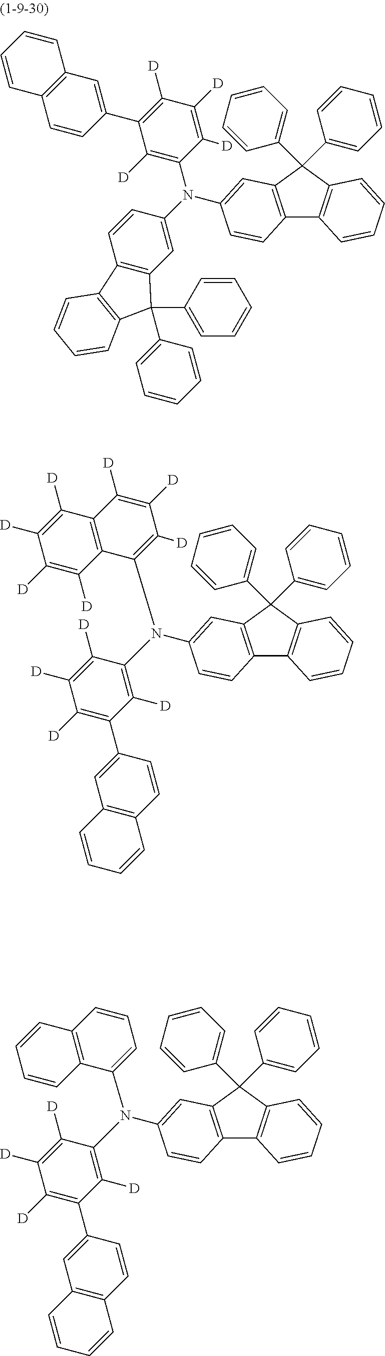 [SMILES: [2H]c1c([2H])c(-c2ccc3ccccc3c2)c([2H])c(N(c2ccc3c(c2)C(c2ccccc2)(c2ccccc2)c2ccccc2-3)c2ccc3c(c2)C(c2ccccc2)(c2ccccc2)c2ccccc2-3)c1[2H] ; [2H]c1c([2H])c(-c2ccc3ccccc3c2)c([2H])c(N(c2ccc3c(c2)C(c2ccccc2)(c2ccccc2)c2ccccc2-3)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc3c(c2)C(c2ccccc2)(c2ccccc2)c2ccccc2-3)c2cccc3ccccc23)c1[2H]]

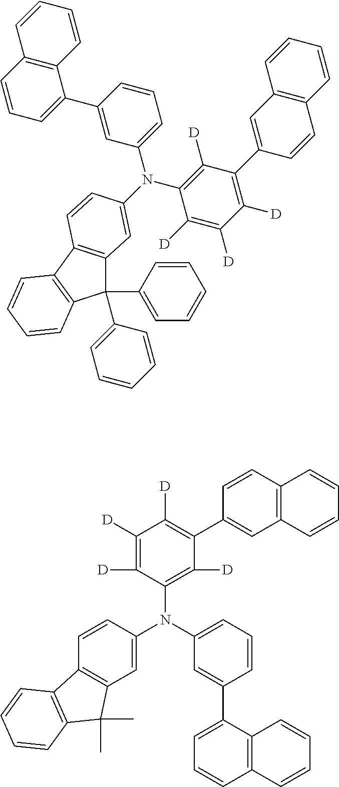 [SMILES: [2H]c1c([2H])c(-c2ccc3ccccc3c2)c([2H])c(N(c2cccc(-c3cccc4ccccc34)c2)c2ccc3c(c2)C(c2ccccc2)(c2ccccc2)c2ccccc2-3)c1[2H] ; [2H]c1c([2H])c(-c2ccc3ccccc3c2)c([2H])c(N(c2cccc(-c3cccc4ccccc34)c2)c2ccc3c(c2)C(C)(C)c2ccccc2-3)c1[2H]]

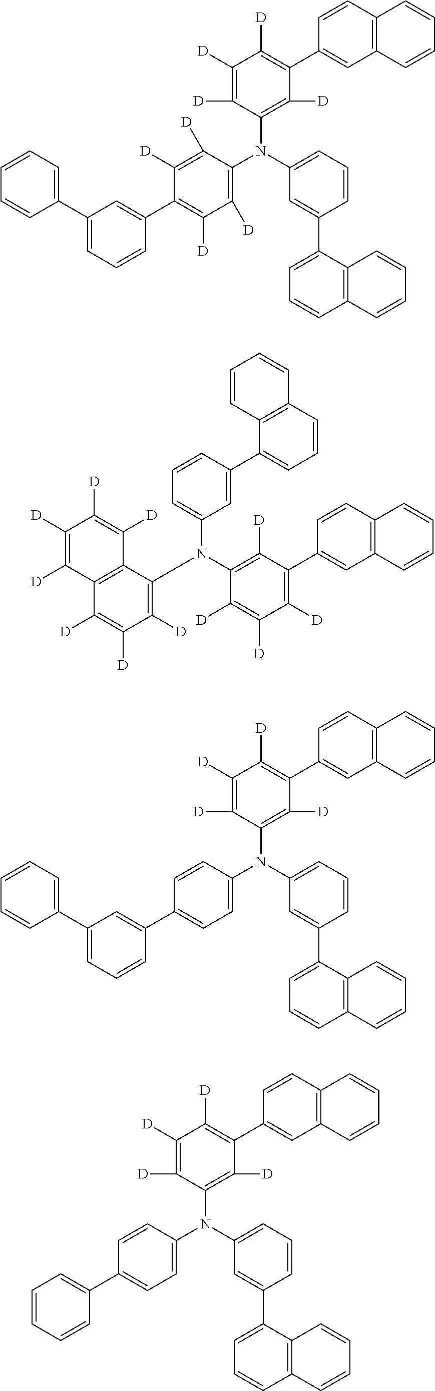 [SMILES: [2H]c1c([2H])c(-c2ccc3ccccc3c2)c([2H])c(N(c2cccc(-c3cccc4ccccc34)c2)c2c([2H])c([2H])c(-c3cccc(-c4ccccc4)c3)c([2H])c2[2H])c1[2H] ; [2H]c1c([2H])c(-c2ccc3ccccc3c2)c([2H])c(N(c2cccc(-c3cccc4ccccc34)c2)c2c([2H])c([2H])c([2H])c3c([2H])c([2H])c([2H])c([2H])c23)c1[2H] ; [2H]c1c([2H])c(-c2ccc3ccccc3c2)c([2H])c(N(c2ccc(-c3cccc(-c4ccccc4)c3)cc2)c2cccc(-c3cccc4ccccc34)c2)c1[2H] ; [2H]c1c([2H])c(-c2ccc3ccccc3c2)c([2H])c(N(c2ccc(-c3ccccc3)cc2)c2cccc(-c3cccc4ccccc34)c2)c1[2H]]

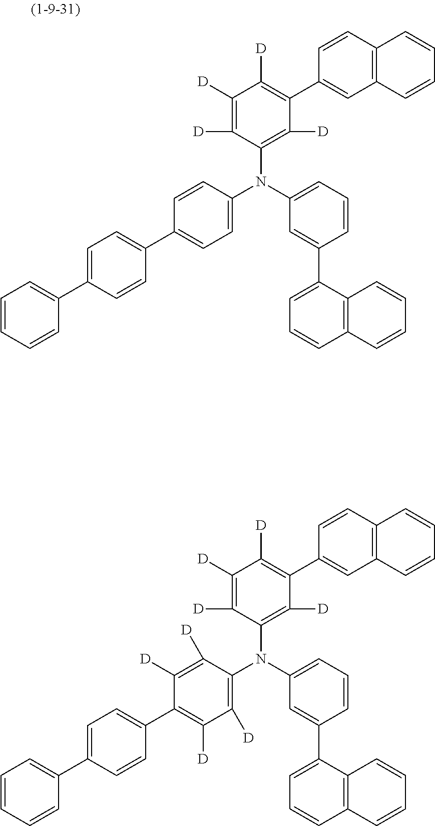 [SMILES: [2H]c1c([2H])c(-c2ccc3ccccc3c2)c([2H])c(N(c2ccc(-c3ccc(-c4ccccc4)cc3)cc2)c2cccc(-c3cccc4ccccc34)c2)c1[2H] ; [2H]c1c([2H])c(-c2ccc3ccccc3c2)c([2H])c(N(c2cccc(-c3cccc4ccccc34)c2)c2c([2H])c([2H])c(-c3ccc(-c4ccccc4)cc3)c([2H])c2[2H])c1[2H]]

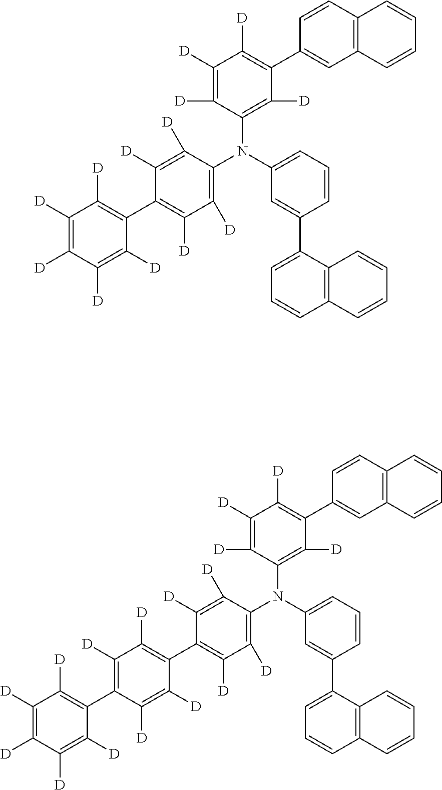 [SMILES: [2H]c1c([2H])c([2H])c(-c2c([2H])c([2H])c(N(c3cccc(-c4cccc5ccccc45)c3)c3c([2H])c([2H])c([2H])c(-c4ccc5ccccc5c4)c3[2H])c([2H])c2[2H])c([2H])c1[2H] ; [2H]c1c([2H])c([2H])c(-c2c([2H])c([2H])c(-c3c([2H])c([2H])c(N(c4cccc(-c5cccc6ccccc56)c4)c4c([2H])c([2H])c([2H])c(-c5ccc6ccccc6c5)c4[2H])c([2H])c3[2H])c([2H])c2[2H])c([2H])c1[2H]]

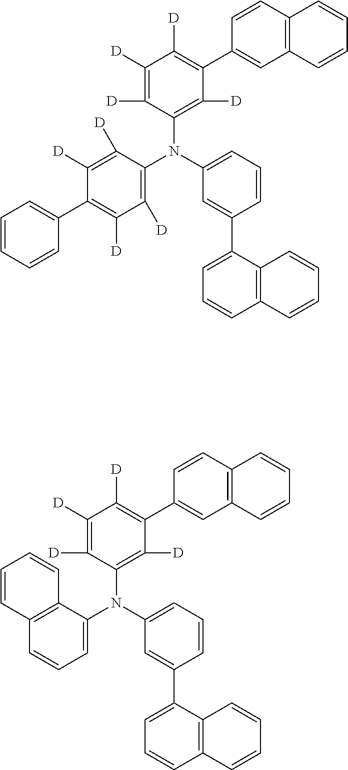 [SMILES: [2H]c1c([2H])c(-c2ccc3ccccc3c2)c([2H])c(N(c2cccc(-c3cccc4ccccc34)c2)c2c([2H])c([2H])c(-c3ccccc3)c([2H])c2[2H])c1[2H] ; [2H]c1c([2H])c(-c2ccc3ccccc3c2)c([2H])c(N(c2cccc(-c3cccc4ccccc34)c2)c2cccc3ccccc23)c1[2H]]

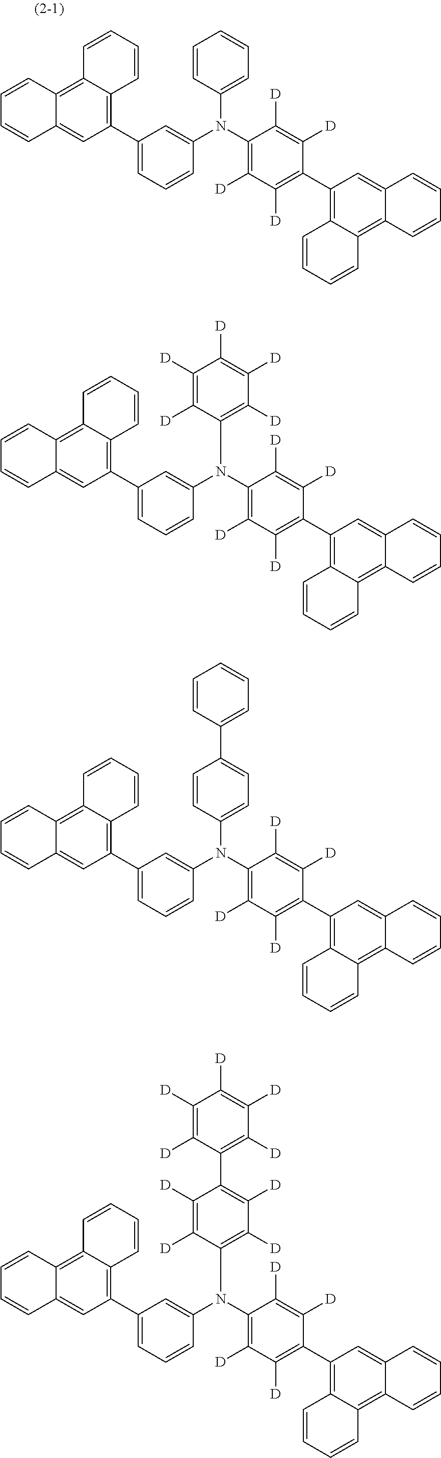 [SMILES: [2H]c1c([2H])c(N(c2ccccc2)c2cccc(-c3cc4ccccc4c4ccccc34)c2)c([2H])c([2H])c1-c1cc2ccccc2c2ccccc12 ; [2H]c1c([2H])c([2H])c(N(c2cccc(-c3cc4ccccc4c4ccccc34)c2)c2c([2H])c([2H])c(-c3cc4ccccc4c4ccccc34)c([2H])c2[2H])c([2H])c1[2H] ; [2H]c1c([2H])c(N(c2ccc(-c3ccccc3)cc2)c2cccc(-c3cc4ccccc4c4ccccc34)c2)c([2H])c([2H])c1-c1cc2ccccc2c2ccccc12 ; [2H]c1c([2H])c([2H])c(-c2c([2H])c([2H])c(N(c3cccc(-c4cc5ccccc5c5ccccc45)c3)c3c([2H])c([2H])c(-c4cc5ccccc5c5ccccc45)c([2H])c3[2H])c([2H])c2[2H])c([2H])c1[2H]]

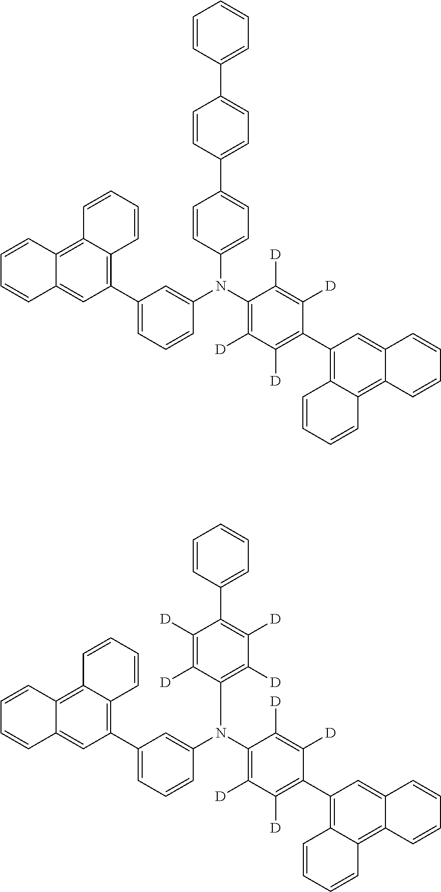 [SMILES: [2H]c1c([2H])c(N(c2ccc(-c3ccc(-c4ccccc4)cc3)cc2)c2cccc(-c3cc4ccccc4c4ccccc34)c2)c([2H])c([2H])c1-c1cc2ccccc2c2ccccc12 ; [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1ccccc1]

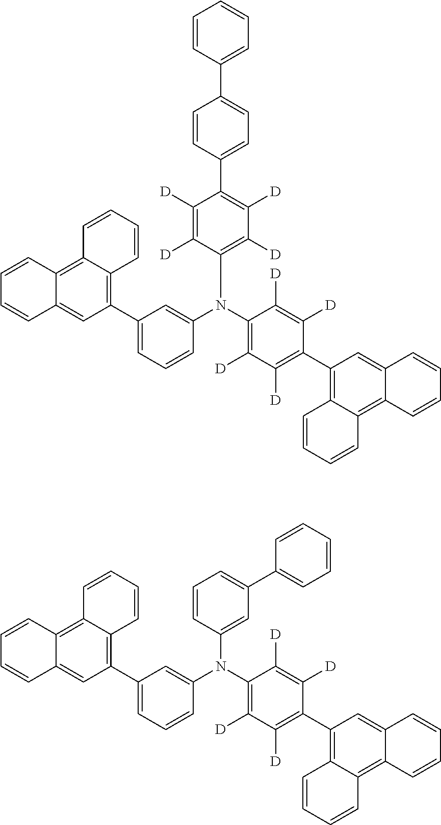 [SMILES: [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1ccc(-c2ccccc2)cc1 ; [2H]c1c([2H])c(N(c2cccc(-c3ccccc3)c2)c2cccc(-c3cc4ccccc4c4ccccc34)c2)c([2H])c([2H])c1-c1cc2ccccc2c2ccccc12]

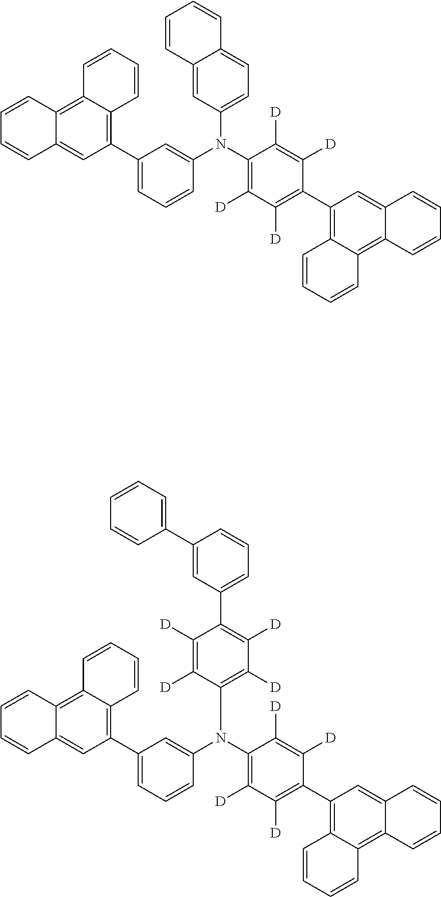 [SMILES: [2H]c1c([2H])c(N(c2cccc(-c3cc4ccccc4c4ccccc34)c2)c2ccc3ccccc3c2)c([2H])c([2H])c1-c1cc2ccccc2c2ccccc12 ; [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1cccc(-c2ccccc2)c1]

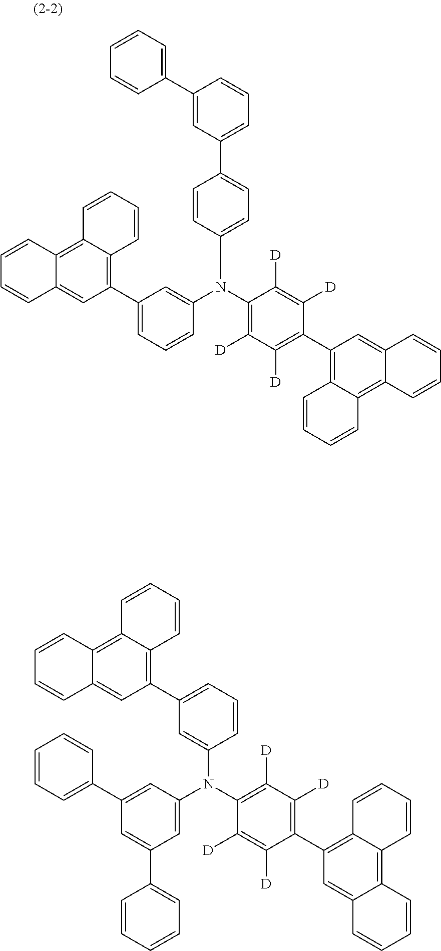 [SMILES: [2H]c1c([2H])c(N(c2ccc(-c3cccc(-c4ccccc4)c3)cc2)c2cccc(-c3cc4ccccc4c4ccccc34)c2)c([2H])c([2H])c1-c1cc2ccccc2c2ccccc12 ; [2H]c1c([2H])c(N(c2cc(-c3ccccc3)cc(-c3ccccc3)c2)c2cccc(-c3cc4ccccc4c4ccccc34)c2)c([2H])c([2H])c1-c1cc2ccccc2c2ccccc12]

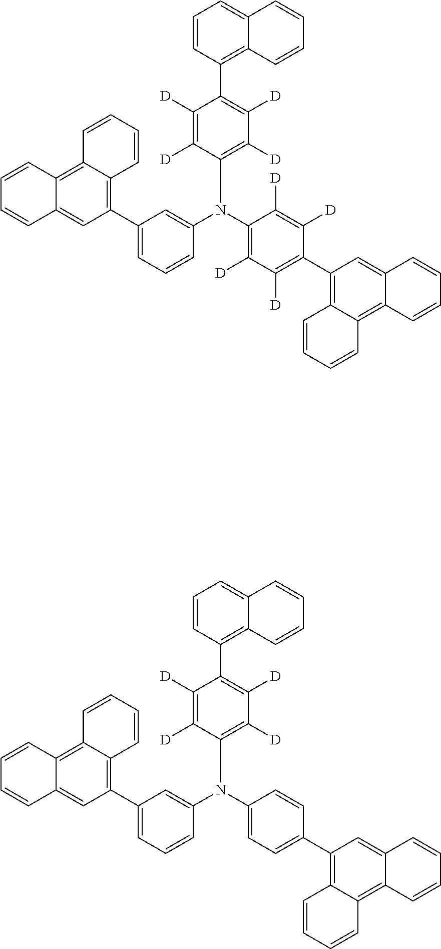 [SMILES: [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1cccc2ccccc12 ; [2H]c1c([2H])c(N(c2ccc(-c3cc4ccccc4c4ccccc34)cc2)c2cccc(-c3cc4ccccc4c4ccccc34)c2)c([2H])c([2H])c1-c1cccc2ccccc12]

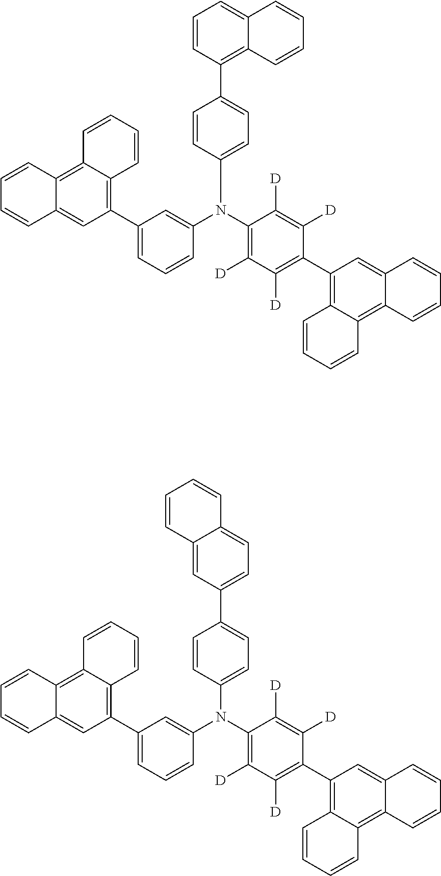 [SMILES: [2H]c1c([2H])c(N(c2ccc(-c3cccc4ccccc34)cc2)c2cccc(-c3cc4ccccc4c4ccccc34)c2)c([2H])c([2H])c1-c1cc2ccccc2c2ccccc12 ; [2H]c1c([2H])c(N(c2ccc(-c3ccc4ccccc4c3)cc2)c2cccc(-c3cc4ccccc4c4ccccc34)c2)c([2H])c([2H])c1-c1cc2ccccc2c2ccccc12]

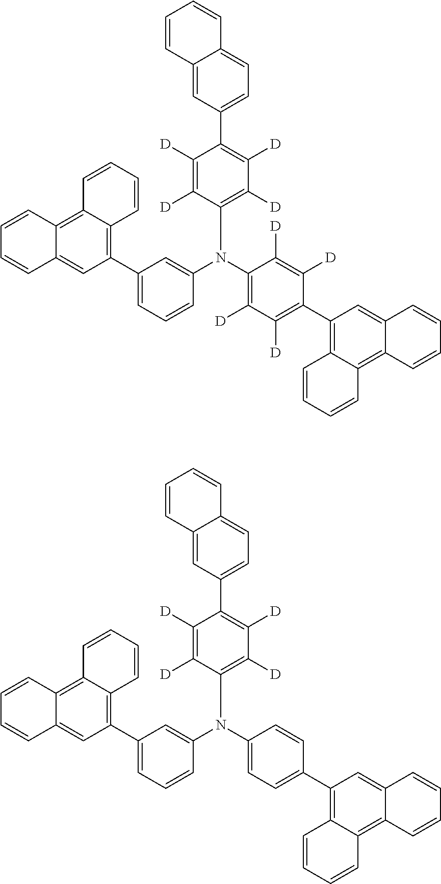 [SMILES: [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1ccc2ccccc2c1 ; [2H]c1c([2H])c(N(c2ccc(-c3cc4ccccc4c4ccccc34)cc2)c2cccc(-c3cc4ccccc4c4ccccc34)c2)c([2H])c([2H])c1-c1ccc2ccccc2c1]

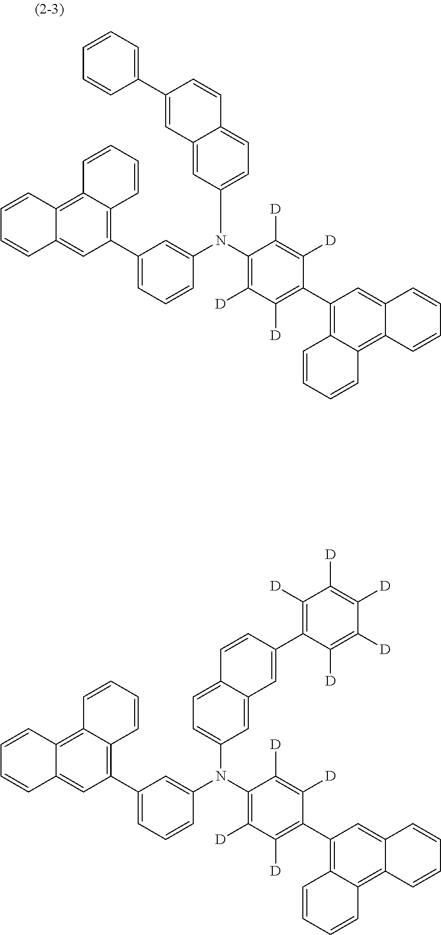 [SMILES: [2H]c1c([2H])c(N(c2cccc(-c3cc4ccccc4c4ccccc34)c2)c2ccc3ccc(-c4ccccc4)cc3c2)c([2H])c([2H])c1-c1cc2ccccc2c2ccccc12 ; [2H]c1c([2H])c([2H])c(-c2ccc3ccc(N(c4cccc(-c5cc6ccccc6c6ccccc56)c4)c4c([2H])c([2H])c(-c5cc6ccccc6c6ccccc56)c([2H])c4[2H])cc3c2)c([2H])c1[2H]]

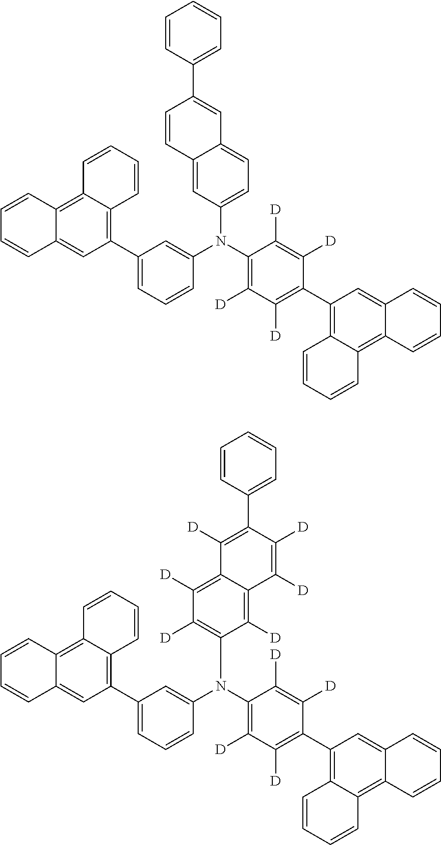 [SMILES: [2H]c1c([2H])c(N(c2cccc(-c3cc4ccccc4c4ccccc34)c2)c2ccc3cc(-c4ccccc4)ccc3c2)c([2H])c([2H])c1-c1cc2ccccc2c2ccccc12 ; [2H]c1c([2H])c(N(c2cccc(-c3cc4ccccc4c4ccccc34)c2)c2c([2H])c([2H])c3c([2H])c(-c4ccccc4)c([2H])c([2H])c3c2[2H])c([2H])c([2H])c1-c1cc2ccccc2c2ccccc12]

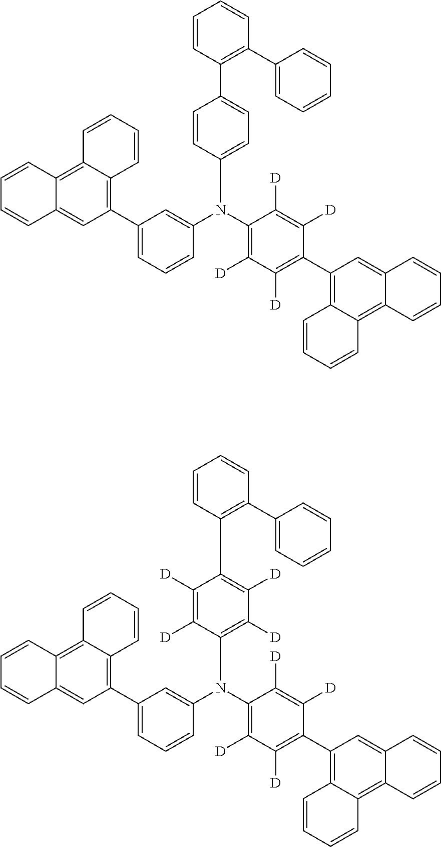 [SMILES: [2H]c1c([2H])c(N(c2ccc(-c3ccccc3-c3ccccc3)cc2)c2cccc(-c3cc4ccccc4c4ccccc34)c2)c([2H])c([2H])c1-c1cc2ccccc2c2ccccc12 ; [2H]c1c([2H])c(N(c2cccc(-c3cc4ccccc4c4ccccc34)c2)c2c([2H])c([2H])c(-c3cc4ccccc4c4ccccc34)c([2H])c2[2H])c([2H])c([2H])c1-c1ccccc1-c1ccccc1]

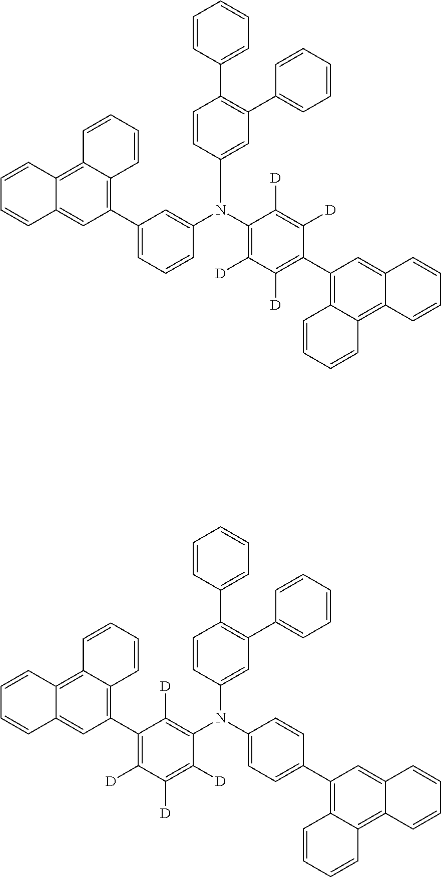 [SMILES: [2H]c1c([2H])c(N(c2cccc(-c3cc4ccccc4c4ccccc34)c2)c2ccc(-c3ccccc3)c(-c3ccccc3)c2)c([2H])c([2H])c1-c1cc2ccccc2c2ccccc12 ; [2H]c1c([2H])c(-c2cc3ccccc3c3ccccc23)c([2H])c(N(c2ccc(-c3cc4ccccc4c4ccccc34)cc2)c2ccc(-c3ccccc3)c(-c3ccccc3)c2)c1[2H]]

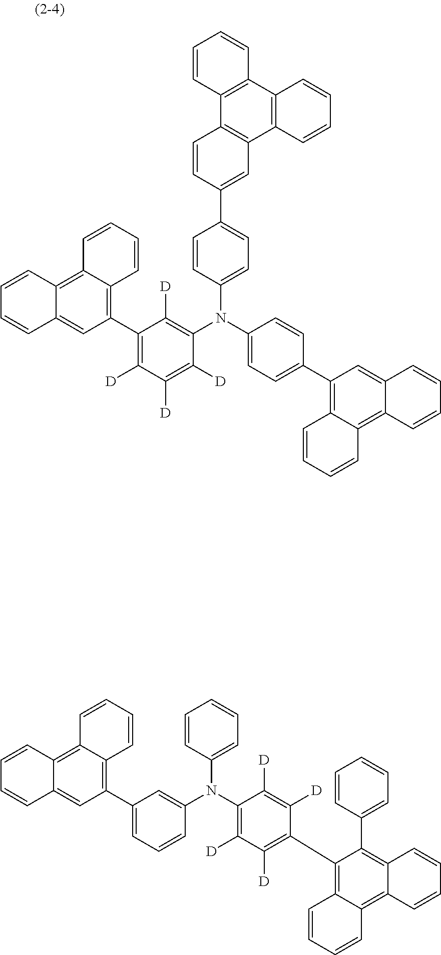 [SMILES: [2H]c1c([2H])c(-c2cc3ccccc3c3ccccc23)c([2H])c(N(c2ccc(-c3ccc4c5ccccc5c5ccccc5c4c3)cc2)c2ccc(-c3cc4ccccc4c4ccccc34)cc2)c1[2H] ; [2H]c1c([2H])c(N(c2ccccc2)c2cccc(-c3cc4ccccc4c4ccccc34)c2)c([2H])c([2H])c1-c1c(-c2ccccc2)c2ccccc2c2ccccc12]

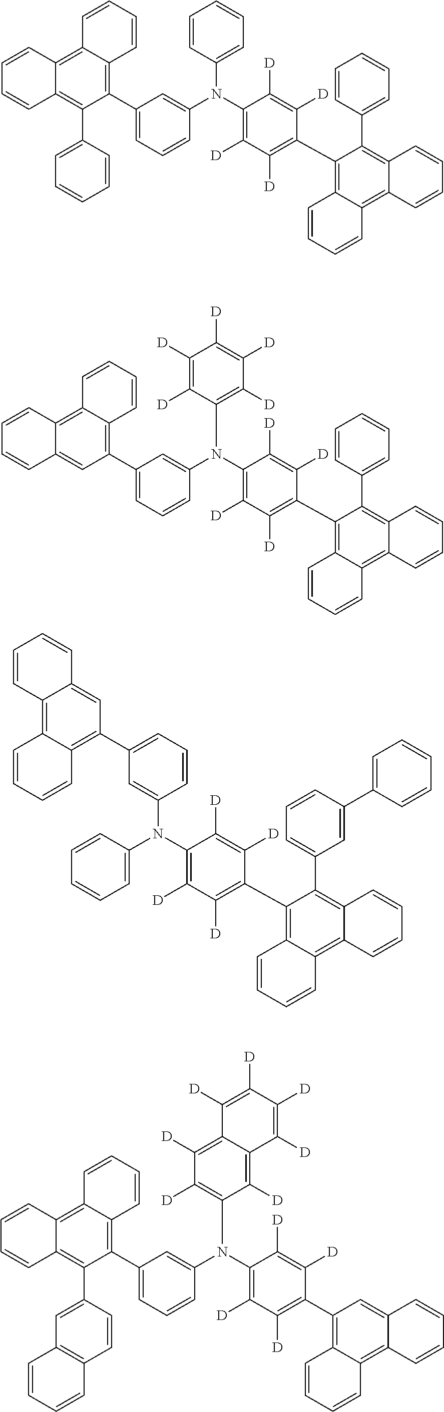

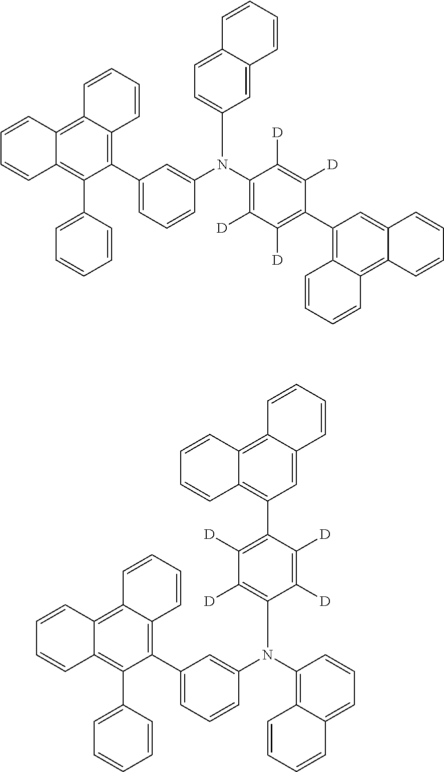 [SMILES: [2H]c1c([2H])c(N(c2cccc(-c3c(-c4ccccc4)c4ccccc4c4ccccc34)c2)c2ccc3ccccc3c2)c([2H])c([2H])c1-c1cc2ccccc2c2ccccc12 ; [2H]c1c([2H])c(N(c2cccc(-c3c(-c4ccccc4)c4ccccc4c4ccccc34)c2)c2cccc3ccccc23)c([2H])c([2H])c1-c1cc2ccccc2c2ccccc12]

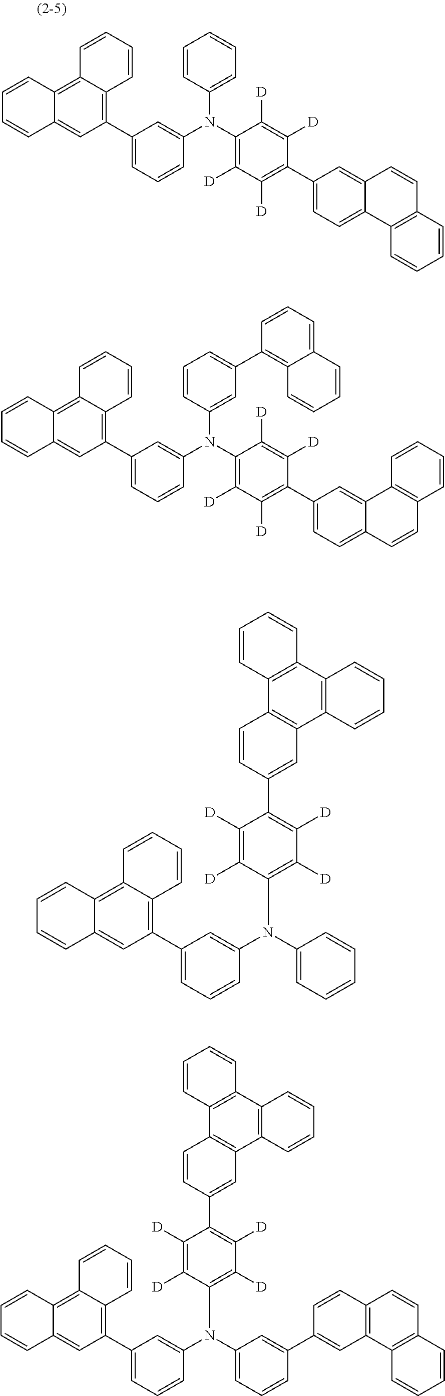 [SMILES: [2H]c1c([2H])c(N(c2ccccc2)c2cccc(-c3cc4ccccc4c4ccccc34)c2)c([2H])c([2H])c1-c1ccc2c(ccc3ccccc32)c1 ; [2H]c1c([2H])c(N(c2cccc(-c3cccc4ccccc34)c2)c2cccc(-c3cc4ccccc4c4ccccc34)c2)c([2H])c([2H])c1-c1ccc2ccc3ccccc3c2c1 ; [2H]c1c([2H])c(N(c2ccccc2)c2cccc(-c3cc4ccccc4c4ccccc34)c2)c([2H])c([2H])c1-c1ccc2c3ccccc3c3ccccc3c2c1 ; [2H]c1c([2H])c(N(c2cccc(-c3ccc4ccc5ccccc5c4c3)c2)c2cccc(-c3cc4ccccc4c4ccccc34)c2)c([2H])c([2H])c1-c1ccc2c3ccccc3c3ccccc3c2c1]

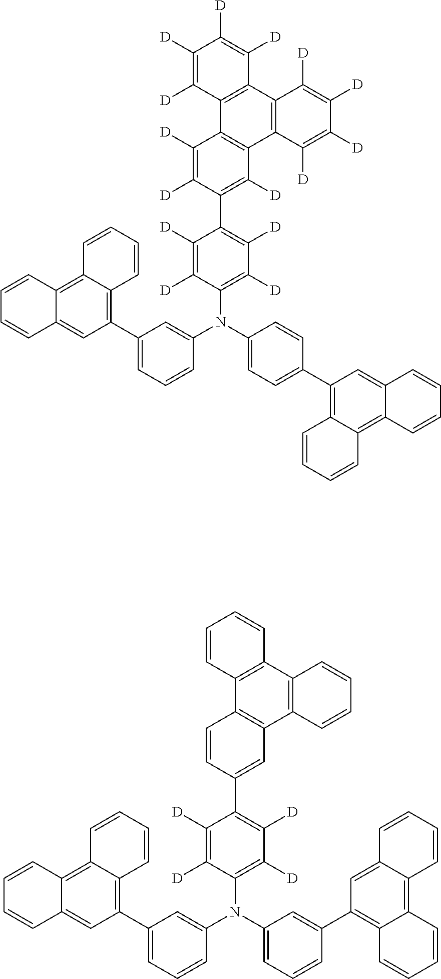 [SMILES: [2H]c1c([2H])c(N(c2ccc(-c3cc4ccccc4c4ccccc34)cc2)c2cccc(-c3cc4ccccc4c4ccccc34)c2)c([2H])c([2H])c1-c1c([2H])c([2H])c2c3c([2H])c([2H])c([2H])c([2H])c3c3c([2H])c([2H])c([2H])c([2H])c3c2c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2c3ccccc3c3ccccc3c2c1]

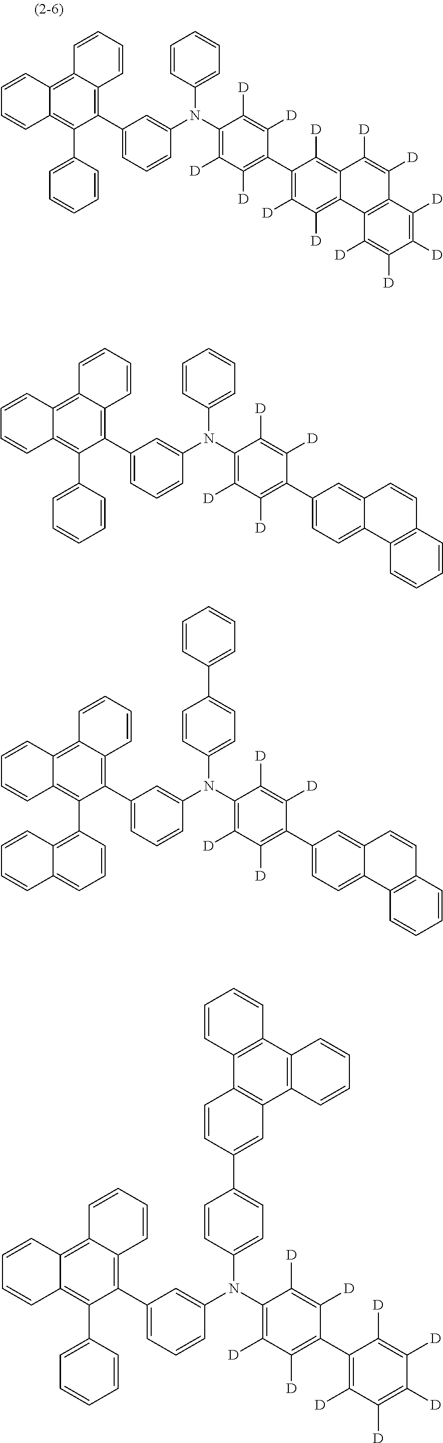

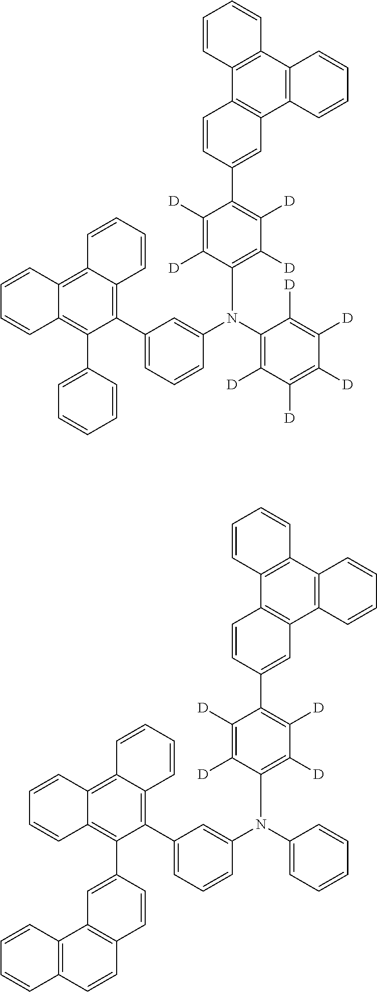 [SMILES: [2H]c1c([2H])c([2H])c(N(c2cccc(-c3c(-c4ccccc4)c4ccccc4c4ccccc34)c2)c2c([2H])c([2H])c(-c3ccc4c5ccccc5c5ccccc5c4c3)c([2H])c2[2H])c([2H])c1[2H] ; [2H]c1c([2H])c(N(c2ccccc2)c2cccc(-c3c(-c4ccc5ccc6ccccc6c5c4)c4ccccc4c4ccccc34)c2)c([2H])c([2H])c1-c1ccc2c3ccccc3c3ccccc3c2c1]

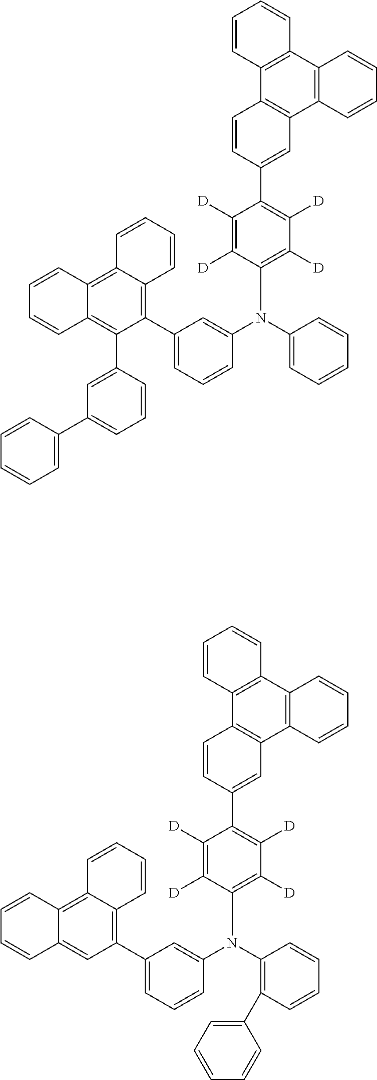 [SMILES: [2H]c1c([2H])c(N(c2ccccc2)c2cccc(-c3c(-c4cccc(-c5ccccc5)c4)c4ccccc4c4ccccc34)c2)c([2H])c([2H])c1-c1ccc2c3ccccc3c3ccccc3c2c1 ; [2H]c1c([2H])c(N(c2cccc(-c3cc4ccccc4c4ccccc34)c2)c2ccccc2-c2ccccc2)c([2H])c([2H])c1-c1ccc2c3ccccc3c3ccccc3c2c1]

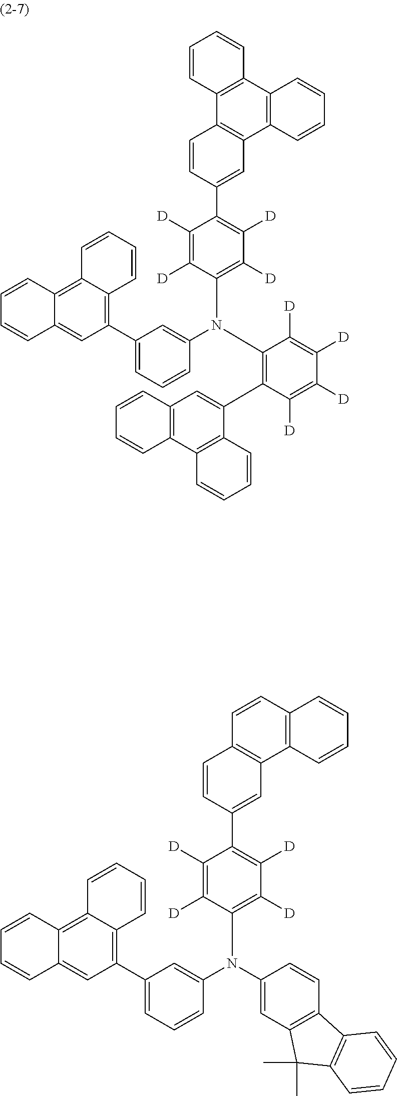 [SMILES: [2H]c1c([2H])c([2H])c(N(c2cccc(-c3cc4ccccc4c4ccccc34)c2)c2c([2H])c([2H])c(-c3ccc4c5ccccc5c5ccccc5c4c3)c([2H])c2[2H])c(-c2cc3ccccc3c3ccccc23)c1[2H] ; [2H]c1c([2H])c(N(c2cccc(-c3cc4ccccc4c4ccccc34)c2)c2ccc3c(c2)C(C)(C)c2ccccc2-3)c([2H])c([2H])c1-c1ccc2ccc3ccccc3c2c1]

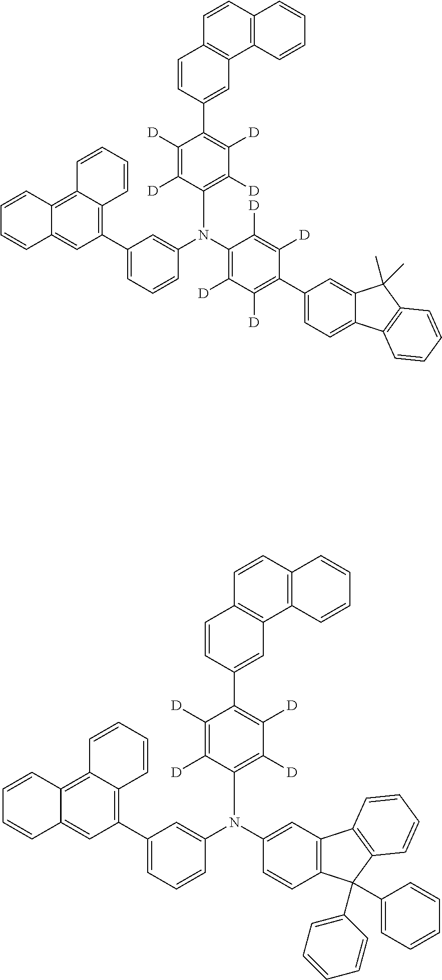 [SMILES: [2H]c1c([2H])c(N(c2cccc(-c3cc4ccccc4c4ccccc34)c2)c2c([2H])c([2H])c(-c3ccc4ccc5ccccc5c4c3)c([2H])c2[2H])c([2H])c([2H])c1-c1ccc2c(c1)C(C)(C)c1ccccc1-2 ; [2H]c1c([2H])c(N(c2cccc(-c3cc4ccccc4c4ccccc34)c2)c2ccc3c(c2)-c2ccccc2C3(c2ccccc2)c2ccccc2)c([2H])c([2H])c1-c1ccc2ccc3ccccc3c2c1]

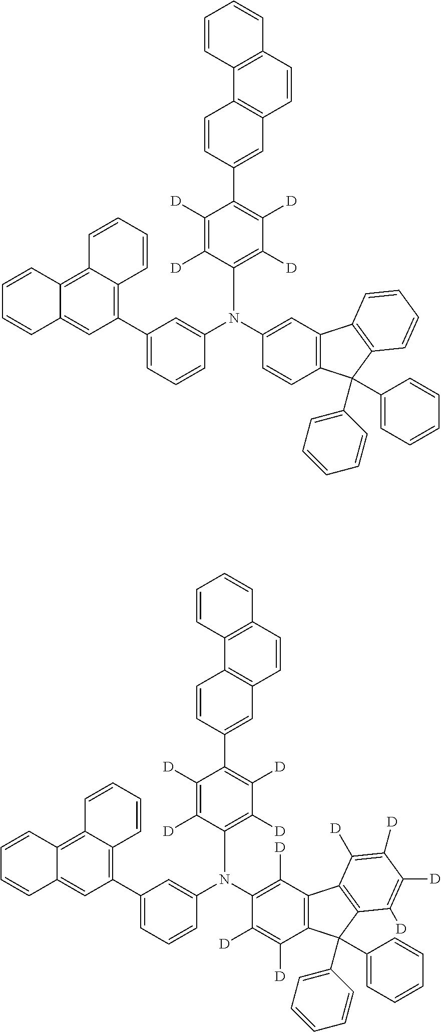 [SMILES: [2H]c1c([2H])c(N(c2cccc(-c3cc4ccccc4c4ccccc34)c2)c2ccc3c(c2)-c2ccccc2C3(c2ccccc2)c2ccccc2)c([2H])c([2H])c1-c1ccc2c(ccc3ccccc32)c1 ; [2H]c1c([2H])c([2H])c2c(c1[2H])-c1c([2H])c(N(c3cccc(-c4cc5ccccc5c5ccccc45)c3)c3c([2H])c([2H])c(-c4ccc5c(ccc6ccccc65)c4)c([2H])c3[2H])c([2H])c([2H])c1C2(c1ccccc1)c1ccccc1]

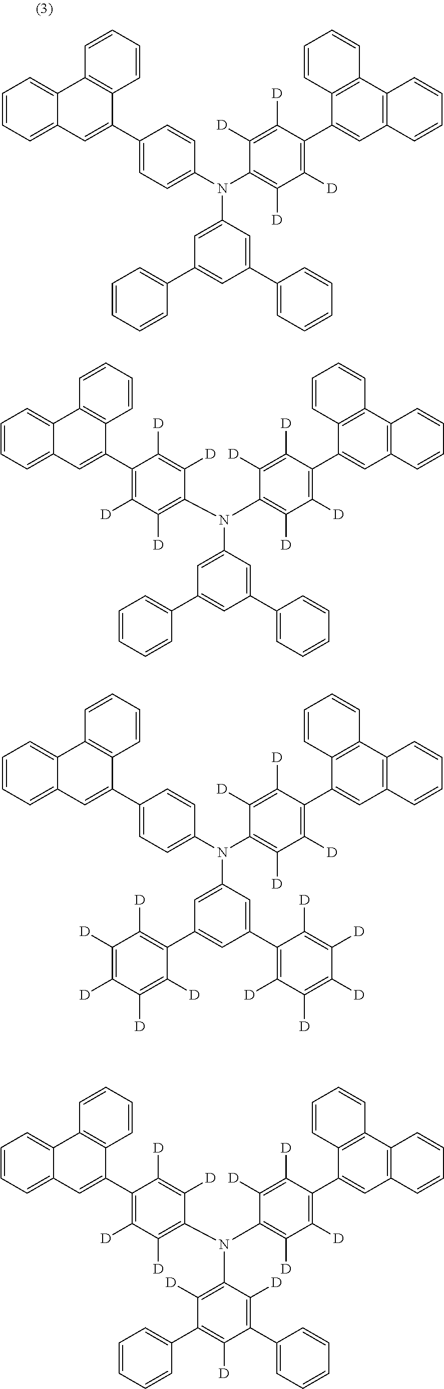

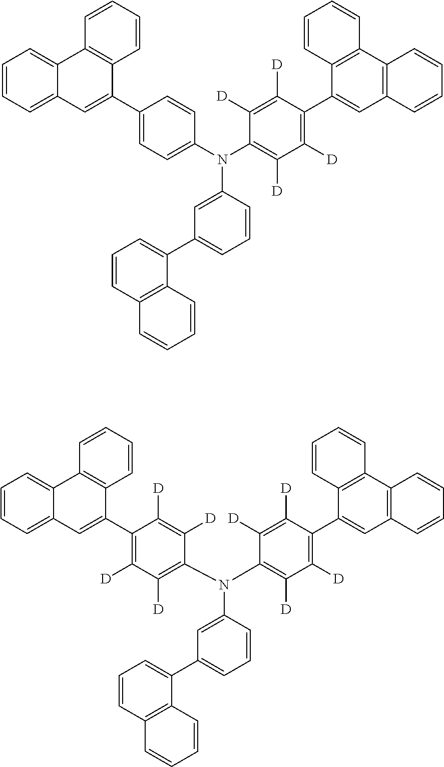 [SMILES: [2H]c1c([2H])c(N(c2ccc(-c3cc4ccccc4c4ccccc34)cc2)c2cccc(-c3cccc4ccccc34)c2)c([2H])c([2H])c1-c1cc2ccccc2c2ccccc12 ; [2H]c1c([2H])c(N(c2cccc(-c3cccc4ccccc34)c2)c2c([2H])c([2H])c(-c3cc4ccccc4c4ccccc34)c([2H])c2[2H])c([2H])c([2H])c1-c1cc2ccccc2c2ccccc12]

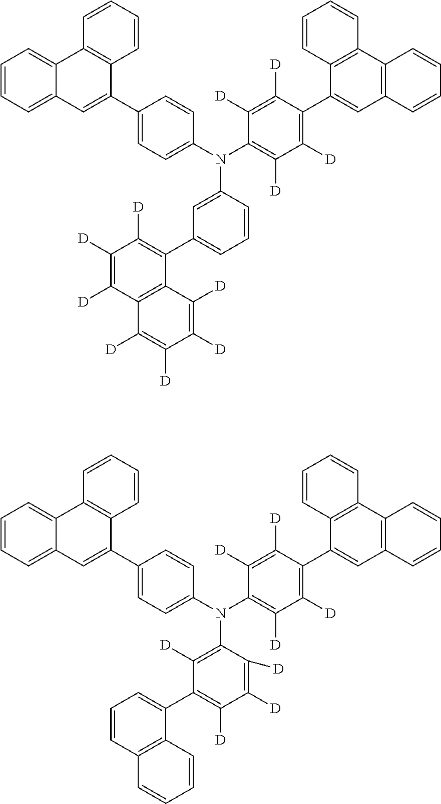 [SMILES: [2H]c1c([2H])c(N(c2ccc(-c3cc4ccccc4c4ccccc34)cc2)c2cccc(-c3c([2H])c([2H])c([2H])c4c([2H])c([2H])c([2H])c([2H])c34)c2)c([2H])c([2H])c1-c1cc2ccccc2c2ccccc12 ; [2H]c1c([2H])c(-c2cccc3ccccc23)c([2H])c(N(c2ccc(-c3cc4ccccc4c4ccccc34)cc2)c2c([2H])c([2H])c(-c3cc4ccccc4c4ccccc34)c([2H])c2[2H])c1[2H]]

### <Second Compound>

As described above, the second compound contained in at least one layer of the organic layer contains 1 mass % or more of a compound having one or more deuterium atoms in the compound represented by the formula (2) (the compound

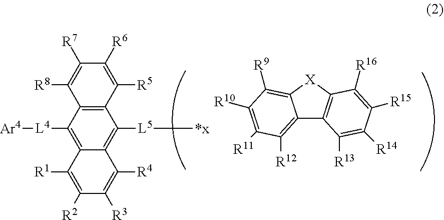

In the formula (2), X is an oxygen atom, a sulfur atom, or NR¹⁰⁰.

In the formula (2), L⁴ and L⁵ each independently represent a single bond, a substituted or unsubstituted arylene group having 6 to 30 ring carbon atoms, or a substituted or unsubstituted divalent heterocyclic group having 5 to 30 ring atoms.

In the formula (2), Ar⁴ represents a substituted or unsubstituted aryl group having 6 to 30 ring carbon atoms or a substituted or unsubstituted monovalent heterocyclic group having 5 to 30 ring atoms.

In the formula (2), one of R⁹ to R¹⁶ and R¹⁰⁰ is bonded to *x.

In the formula (2), R¹ to R⁸, and R⁹ to R¹⁶ and R¹⁰⁰ that are not bonded to *x each independently represent a hydrogen atom or a substituent A.

The substituent A is a substituted or unsubstituted alkyl group having 1 to 50 carbon atoms or a substituted or unsubstituted aryl group having 6 to 50 ring carbon atoms.

It is preferred that R⁹ to R¹⁶ and R¹⁰⁰ that are not bonded to *x all represent hydrogen atoms, or only one of them represents the substituent A.

In the formula (2), adjacent two selected from R⁹ to R¹⁶ that are not bonded to *x may form a ring or may not form a ring.

Ar⁴ preferably represents a substituted or unsubstituted aryl group having 6 to 30 ring carbon atoms.

Ar⁴ is more preferably selected from groups represented by the following formulae (a1) to (a4).

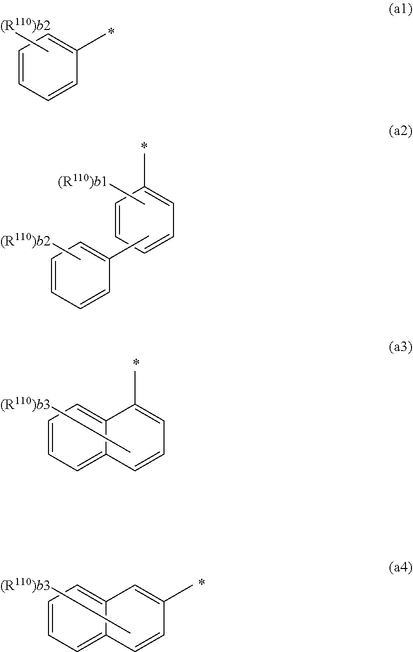

In the formulae (a1) to (a4), * is a single bond bonded to L⁴.
R¹¹⁰ represents
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆) (R₉₀₇),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms.
R₉₀¹ to R⁹⁰⁷ are as defined in the “Substituents in Description”.
b1 is an integer of 0 to 4.
b2 is an integer of 0 to 5.
b3 is an integer of 0 to 7.

When b1 to b3 are each 2 or more, a plurality of R¹¹⁰'s may be the same as or different from each other.

When b1 to b3 are each 2 or more, a plurality of adjacent R¹¹⁰'s are bonded to each other to form a substituted or unsubstituted saturated or unsaturated ring, or do not form a substituted or unsubstituted saturated or unsaturated ring.

L⁴ and L⁵ each independently preferably represent a single bond or a substituted or unsubstituted arylene group having 6 to 14 ring carbon atoms. At least one of L⁴ and L⁵ is preferably a single bond.

In one embodiment, L⁴ and L⁵ each independently represent a single bond or a substituted or unsubstituted arylene group having 6 to 14 ring carbon atoms. At least one of L⁴ and L⁵ is preferably a single bond.

In one preferred aspect of the present invention, the compound (2) is represented by the following formula (2-1).

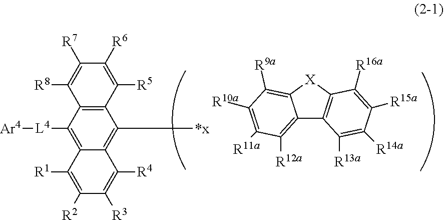

In the formula (2-1),
X represents an oxygen atom, a sulfur atom, or NR¹⁰⁰
one of R^{9a} to R₁₆ₐ and R¹⁰⁰ is bonded to *x¹,
R^{9a} to R^{16a} and R¹⁰⁰ that are not bonded to *x¹ each independently represent a hydrogen atom or a substituent A, and
L⁴, Ar⁴, X, R⁴ to R⁸, and the substituent A are as defined in the formula (2).

In one preferred aspect of the present invention, the compound (2) is represented by any one of the following formula (2-2a) to formula (2-2c).

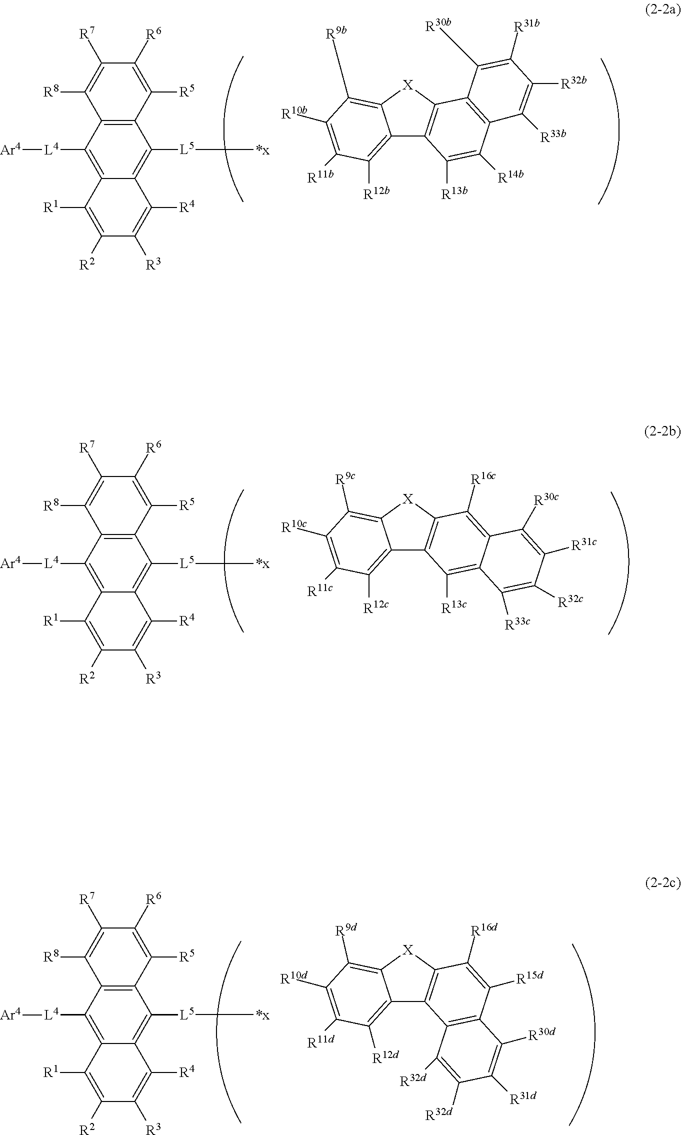

In the formula (2-2a) to the formula (2-2c), X represents an oxygen atom, a sulfur atom, or NR¹⁰⁰.

In the formula (2-2a), one of R^{9b} to R^{14b}, R^{30b} to R³³, and R¹⁰⁰ is bonded to *x. R^{9b} to R^{14b}, R^{30b} to R^{33b}, and R¹⁰⁰ that are not bonded to *x each independently represent a hydrogen atom or a substituent A.

In the formula (2-2b), one of R^{9c} to R^{13c}, R^{16c}, R^{30c} to R^{33c}, and R¹⁰⁰ is bonded to *x. R^{9c} to R^{13c}, R^{16c}, R^{30c} to R^{33c}, and R¹⁰⁰ that are not bonded to *x each independently represent a hydrogen atom or a substituent A.

In the formula (2-2c), one of R^{9d} to R^{12d}, R^{15d}, R^{16d}, R^{30d} to R^{33d}, and R¹⁰⁰ is bonded to *x. R^{9d} to R^{12d}, R^{15d}, R^{16d}, R^{30d} to R^{33d}, and R¹⁰⁰ that are not bonded to *x each independently represent a hydrogen atom or a substituent A.

In the formula (2-2a) to the formula (2-2c), L⁴, L⁵, Ar⁴, and the substituent A are as defined in the formula (2).

In one aspect of the present invention, the compound (2) is represented by the following formula (2-1-1).

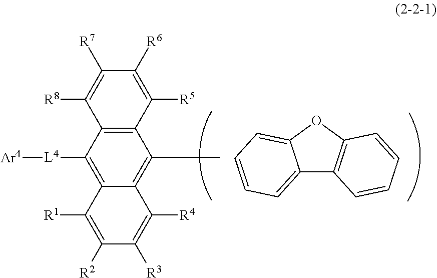

In the formula (2-1-1), L⁴, Ar⁴, and R¹ to R⁸ are as defined in the formula (2).

In one aspect of the present invention, the compound (2) is represented by the following formula (2-1-2).

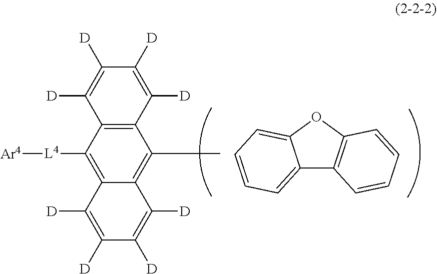

In the formula (2-1-1), L⁴ and Ar⁴ are as defined in the formula (2).

In one aspect of the present invention, the compound (2) is represented by the following formula (2-1-3a) or (2-1-3b).

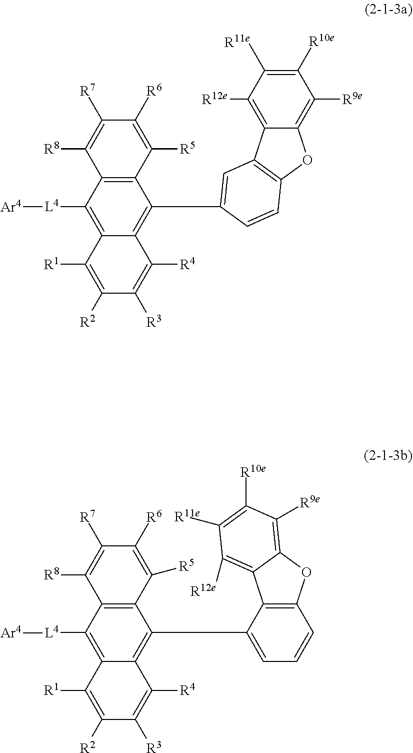

In the formulae (2-1-3a) and (2-1-3b), R^{9e} to R^{12e} each independently represent a hydrogen atom or a substituent A.

In the formulae (2-1-3a) and (2-1-3b), L⁴, Ar⁴, R¹ to R⁸, and the substituent A are as defined in the formula (2).

In a preferred aspect of the present invention, the compound (2) is represented by any one of the following formulae (2-2a-1) to (2-2c-1).

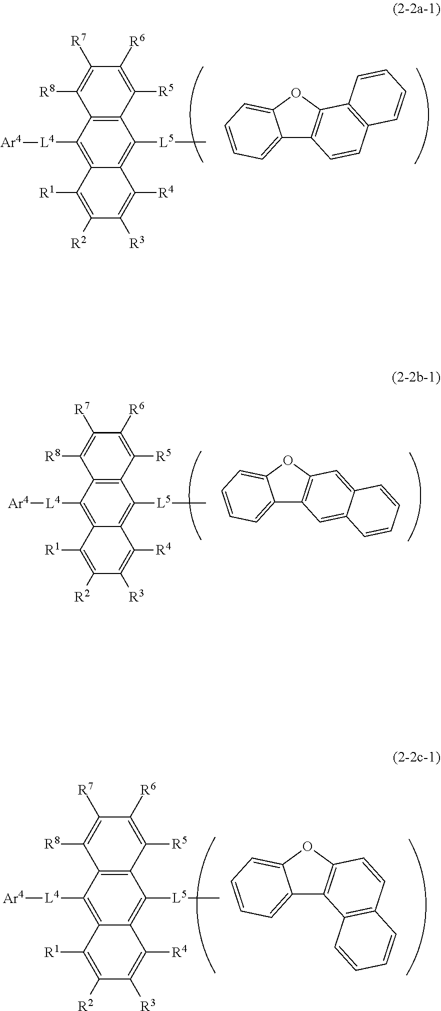

In the formulae (2-2a-1) to (2-2c-1), L⁴, L⁵, Ar⁴, and R¹ to R⁸ are as defined in the formula (2).

In one aspect of the present invention, the compound (2) is represented by the following formulae (2-2a-2) to (2-2c-2).

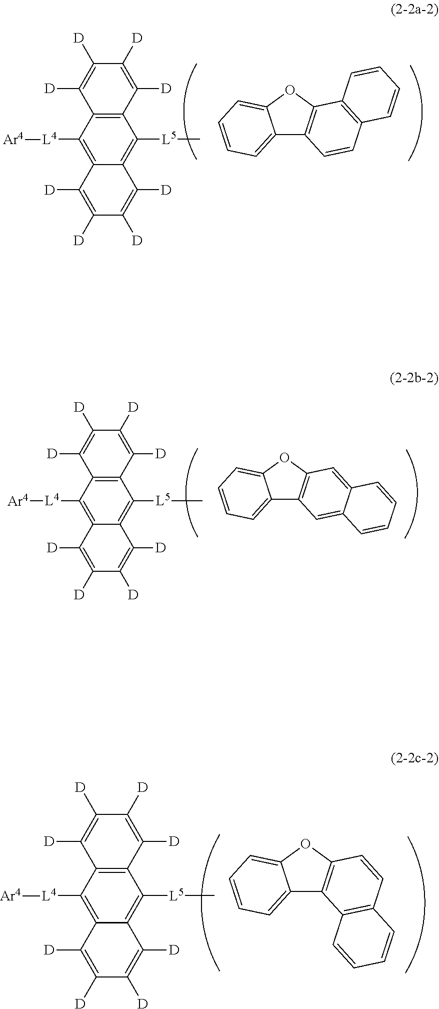

In the formulae (2-2a-2) to (2-2c-2), L⁴, L⁵, and Ar⁴ are as defined in the formula (2).

In one aspect of the present invention, the compound (2) is represented by the following formula (2-2d).

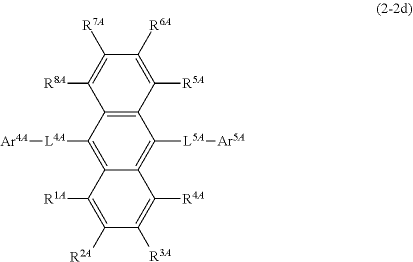

In the formula (2-2d), R^{1A} to R^{8A} each independently represent a hydrogen atom, and at least one of R^{1A} to R^{8A} represents a deuterium atom.

In the formula (2-2d), L^{1A} and L^{2A} each independently represent a single bond, an unsubstituted phenylene group, or an unsubstituted naphthylene group.

In the formula (2-2d), Ar^{4A} represents a phenyl group which may have a phenyl group as a substituent or a naphthyl group which may have a phenyl group as a substituent.

In the formula (2-2d), Ar^{5A} represents a monovalent group represented by the following formula (2A), (3A), or (4A).

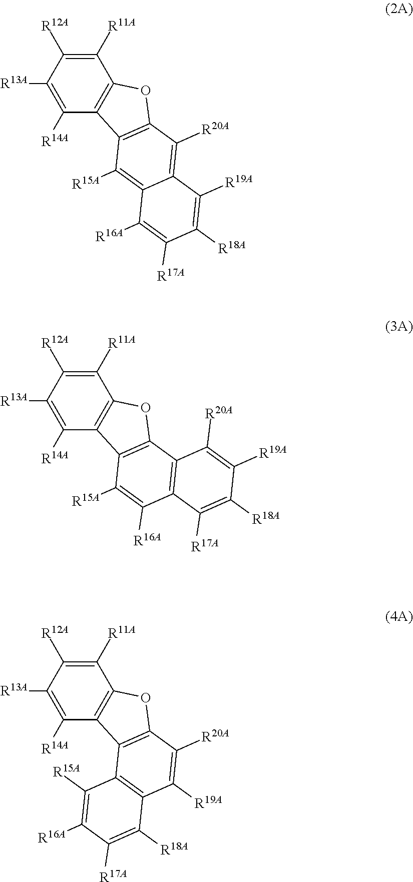

In the formulae (2A) to (4A), either one of R^{13A} and R^{14A} is a single bond bonded to L^{2A}. R^{11A}, R^{12A}, R^{15A} to R^{20A}, and R^{13A} and R^{14A} which are not the single bond bonded to L^{2A} each independently represent a hydrogen atom or an unsubstituted aryl group having 6 to 50 ring carbon atoms.

In one embodiment, in the formula (2-2d), at least two of R^{1A} to R^{8A} represent deuterium atoms.

In one embodiment, in the formula (2-2d), all of R^{1A} to R^{8A} represent deuterium atoms.

In one embodiment, in the formula (2-2d), at least one hydrogen atom of Ar^{1A} represents a deuterium atom.

In one embodiment, in the formula (2-2d), R^{11A}, R^{12A}, R^{15A} to R^{20A}, and R^{13A} and R^{14A} which are not the single bond bonded to L^{2A} represent hydrogen atoms.

In one embodiment, in the formula (2-2d), at least one of R^{11A}, R^{12A}, R^{15A} to R^{20A}, and R^{13A} and R^{14A} which are not the single bond bonded to L^{2A} represents a deuterium atom.

The compound represented by the formula (2) can be synthesized by using a known alternative reaction or raw material suitable for a target product in accordance with a synthesis method described in Examples.

### (Deuterium Atom in Compound (2))

As described above, the “hydrogen atom” used in the description herein encompasses a protium atom, a deuterium atom, and a tritium atom. Therefore, the compound (2) may have a naturally-derived deuterium atom.

A deuterium atom may be intentionally introduced into the compound (2) by using a compound obtained by partially or wholly deuterating a raw material compound. Therefore, in one aspect of the present invention, the compound (2) has at least one deuterium atom. That is, the compound (2) may be a compound represented by the formula (2) in which at least one of hydrogen atoms in the compound is a deuterium atom.

Here, examples of the partially or wholly deuterated raw material compound include a compound forming a terminal portion (Ar⁴, X, and a skeleton portion containing two benzene rings) of the formula (2), and a compound forming a portion having an anthracene skeleton of the formula (2) and a linker portion (L⁴, L⁵) connecting the terminal portion and the portion having an anthracene skeleton of the formula (2).

A deuteration ratio of the compound (2) depends on a deuteration ratio of the raw material compound to be used. Even when a raw material having a predetermined deuteration ratio is used, a naturally-derived protium isotope may still be contained at a certain ratio. Accordingly an aspect of the deuteration ratio of the compound (2) shown below includes a ratio obtained by simply counting the number of deuterium atoms represented in a chemical formula, and a ratio obtained by taking into account a trace amount of naturally-derived isotopes.

The deuteration ratio of the compound (2) contained in the first compound is 1% or more, preferably 3% or more, more preferably 5% or more, even more preferably 10% or more, and still more preferably 50% or more. In other words, the second compound contains 1 mass % or more, preferably 3 mass % or more, more preferably 5 mass % or more, even more preferably 10 mass % or more, and still more preferably 50 mass % or more of the compound having one or more deuterium atoms in the compound represented by the formula (2) (the compound (2)).

The compound (2) may be a mixture containing a deuterated compound and a non-deuterated compound, or a mixture of two or more compounds having different deuteration ratios. For example, the compound (2) may be a mixture of the following compound B1 and compound B2, or a mixture of the following compound C1 and compound C2. In this case, the first compound may contain the following compounds B1 and B2, or the following compounds C1 and C2.

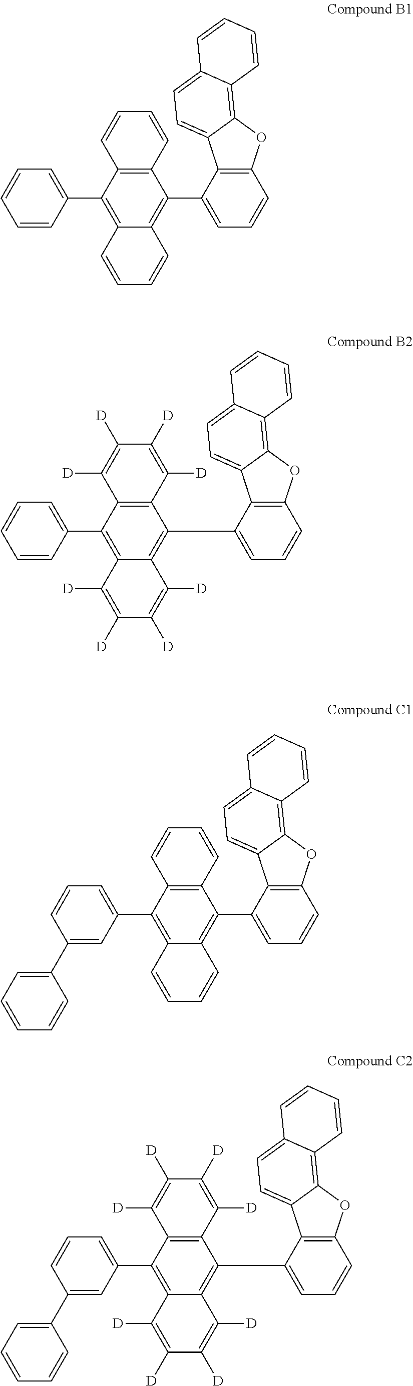 [SMILES: c1ccc(-c2c3ccccc3c(-c3cccc4oc5c6ccccc6ccc5c34)c3ccccc23)cc1 ; [2H]c1c([2H])c([2H])c2c(-c3cccc4oc5c6ccccc6ccc5c34)c3c([2H])c([2H])c([2H])c([2H])c3c(-c3ccccc3)c2c1[2H] ; c1ccc(-c2cccc(-c3c4ccccc4c(-c4cccc5oc6c7ccccc7ccc6c45)c4ccccc34)c2)cc1 ; [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H]]

The compound (2) may be, for example, a mixture of the following compound E1 and compound E2, a mixture of the following compound F1 and compound F2, or a mixture of the following compound G1 and compound G2. The compound (2) may be a mixture of the following compound I1 and compound I2, or a mixture of the following compound J1 and compound J2. In these cases, the first compound may contain the following compounds E1 and E2, F1 and F2, G1 and G2, I1 and I2, or J1 and J2.

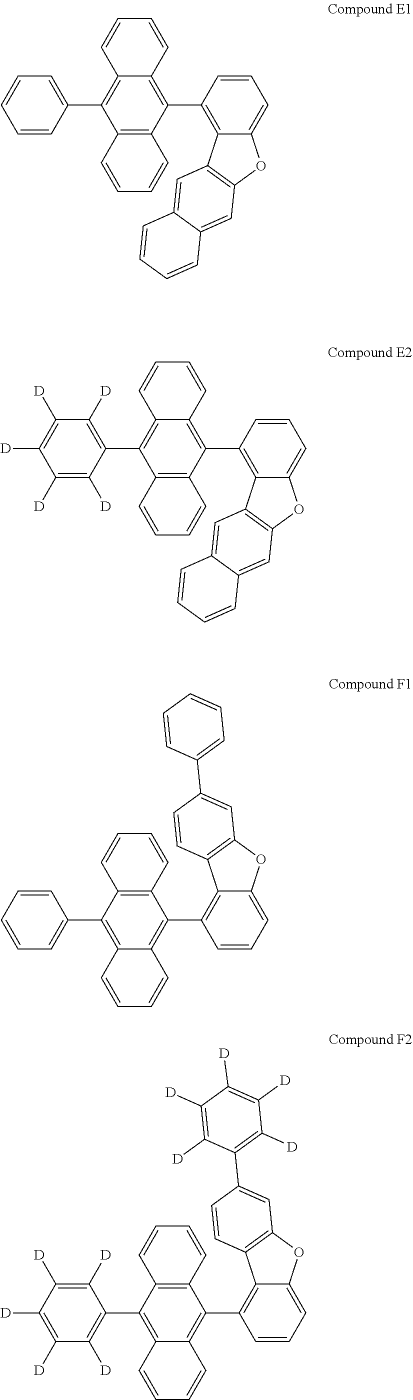 [SMILES: c1ccc(-c2c3ccccc3c(-c3cccc4oc5cc6ccccc6cc5c34)c3ccccc23)cc1 ; [2H]c1c([2H])c([2H])c(-c2c3ccccc3c(-c3cccc4oc5cc6ccccc6cc5c34)c3ccccc23)c([2H])c1[2H] ; c1ccc(-c2ccc3c(c2)oc2cccc(-c4c5ccccc5c(-c5ccccc5)c5ccccc45)c23)cc1 ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

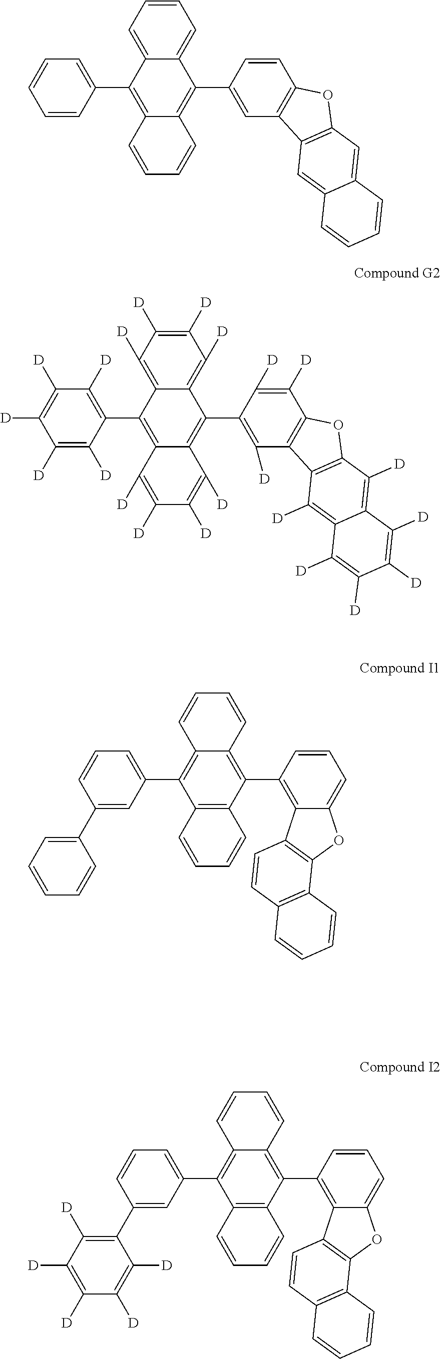 [SMILES: c1ccc(-c2c3ccccc3c(-c3ccc4oc5cc6ccccc6cc5c4c3)c3ccccc23)cc1 ; [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; c1ccc(-c2cccc(-c3c4ccccc4c(-c4cccc5oc6c7ccccc7ccc6c45)c4ccccc34)c2)cc1 ; [2H]c1c([2H])c([2H])c(-c2cccc(-c3c4ccccc4c(-c4cccc5oc6c7ccccc7ccc6c45)c4ccccc34)c2)c([2H])c1[2H]]

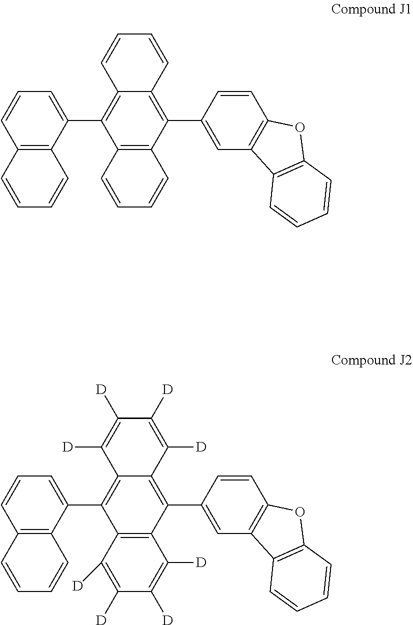 [SMILES: c1ccc2c(-c3c4ccccc4c(-c4ccc5oc6ccccc6c5c4)c4ccccc34)cccc2c1 ; [2H]c1c([2H])c([2H])c2c(-c3cccc4ccccc34)c3c([2H])c([2H])c([2H])c([2H])c3c(-c3ccc4oc5ccccc5c4c3)c2c1[2H]]

A deuteration ratio of such a mixture is preferably 1% or more, more preferably 3% or more, even more preferably 5% or more, still more preferably 10% or more, and even still more preferably 50% or more, and is less than 100%.

A ratio of the number of deuterium atoms to the total number of hydrogen atoms in the compound (2) is preferably 1% or more, more preferably 3% or more, even more preferably 5% or more, and still more preferably 10% or more, and is 100% or less.

In one aspect of the organic EL element, the compound (1) contains the compounds A1 and A2, and the compound (2) contains the compounds B1 and B2 or the compounds C1 and C2.

In another aspect of the organic EL element, the compound (1) contains the compounds D1 and D2, and the compound (2) contains the compounds E1 and E2, the compounds F1 and F2, or the compounds G1 and G2.

In still another aspect of the organic EL element, the compound (1) contains the compounds H1 and H2, and the compound (2) contains the compounds I1 and I2, or the compounds J1 and J2.

Specific examples of the compound (2) according to the embodiment of the present invention are shown below. In the following specific examples, “D” represents a deuterium atom. The compound (2) according to the embodiment of the present invention is not limited to these specific examples.

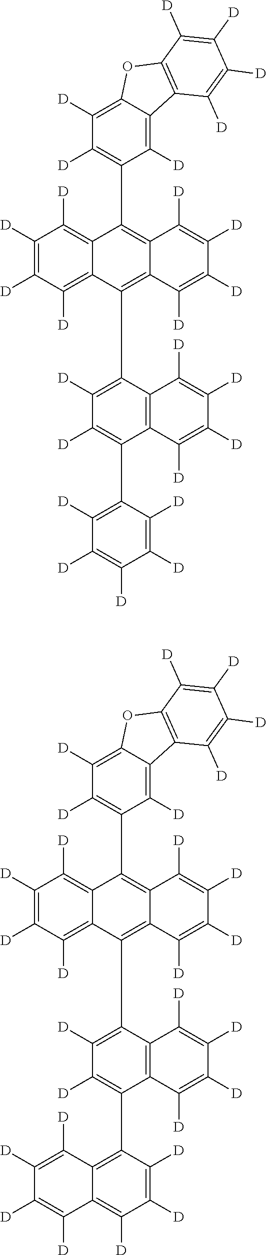 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c23)c([2H])c1[2H] ; [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c([2H])c7c([2H])c([2H])c([2H])c([2H])c67)c6c([2H])c([2H])c([2H])c([2H])c56)c5c([2H])c([2H])c([2H])c([2H])c45)c([2H])c32)c1[2H]]

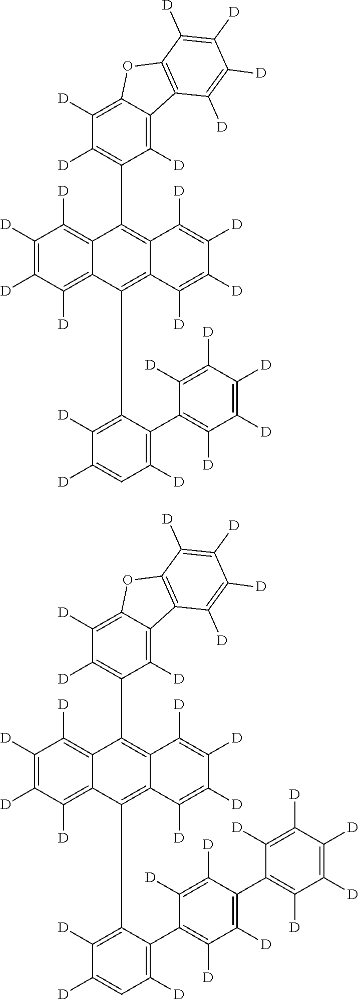

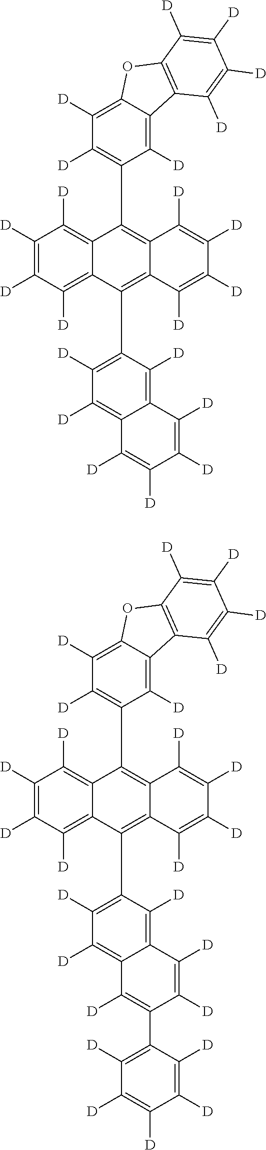 [SMILES: [2H]c1c([2H])c([2H])c2c(oc3c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c([2H])c3c2[2H])c([2H])c1[2H]]

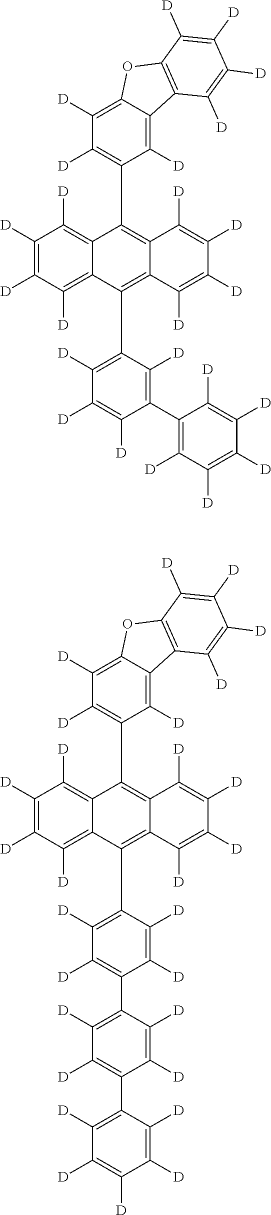 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c3[2H])c([2H])c2[2H])c([2H])c1[2H]]

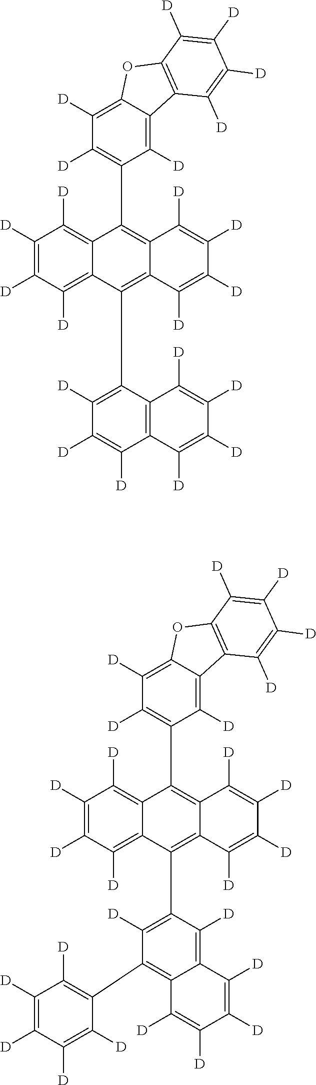 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c3c([2H])c([2H])c([2H])c([2H])c23)c([2H])c1[2H]]

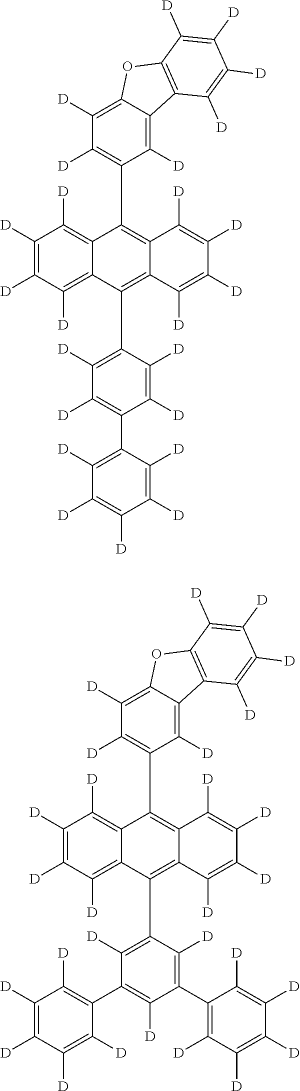 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H]]

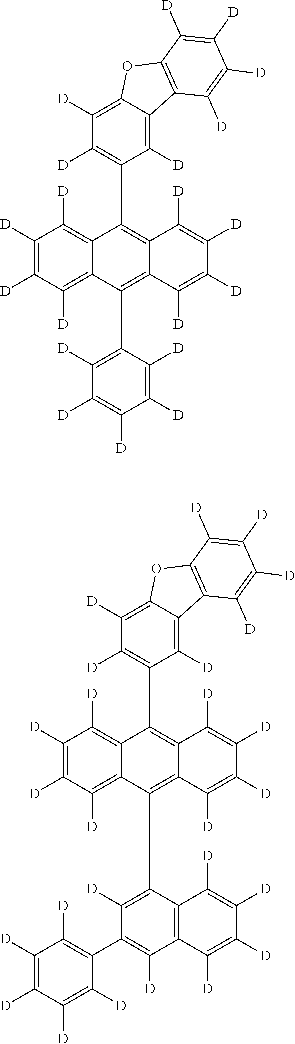 [SMILES: [2H]c1c([2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c3c2[2H])c([2H])c1[2H]]

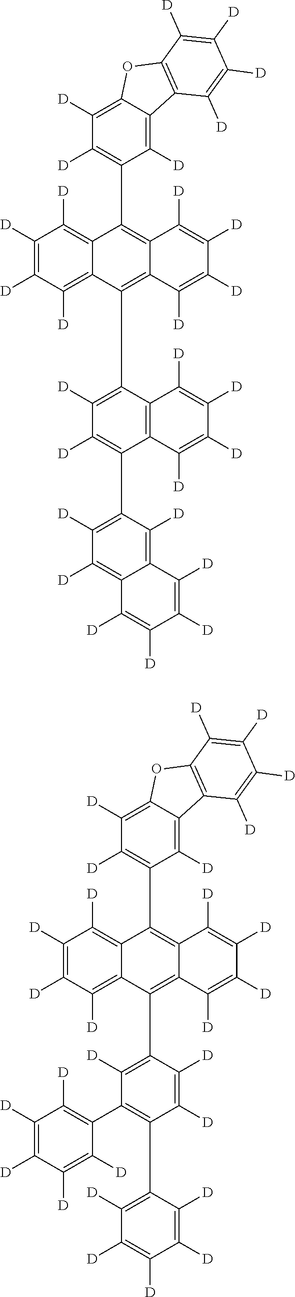 [SMILES: [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2-c2c([2H])c([2H])c([2H])c([2H])c2[2H])c([2H])c1[2H]]

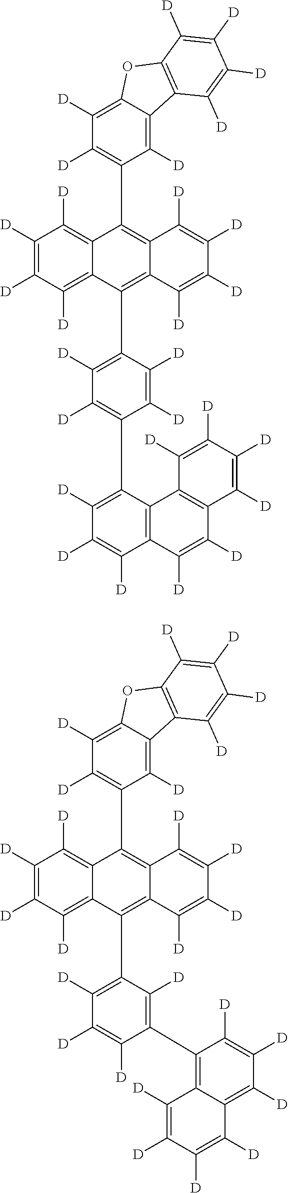

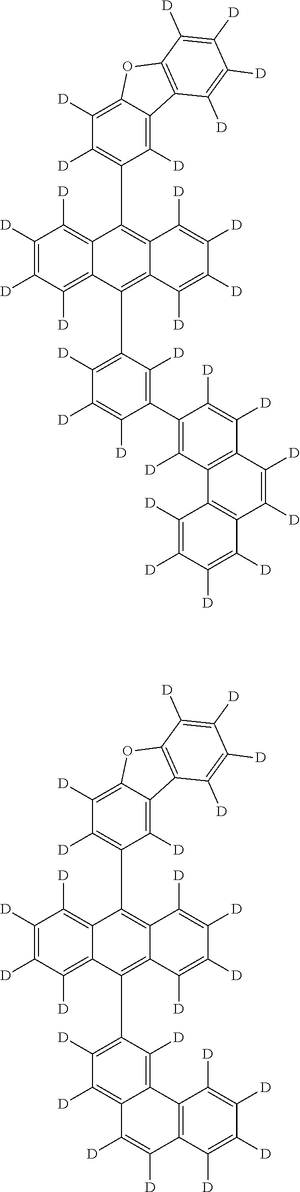 [SMILES: [2H]c1c([2H])c(-c2c([2H])c([2H])c3c([2H])c([2H])c4c([2H])c([2H])c([2H])c([2H])c4c3c2[2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c1[2H] ; [2H]c1c([2H])c([2H])c2c(oc3c([2H])c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c([2H])c32)c1[2H]]

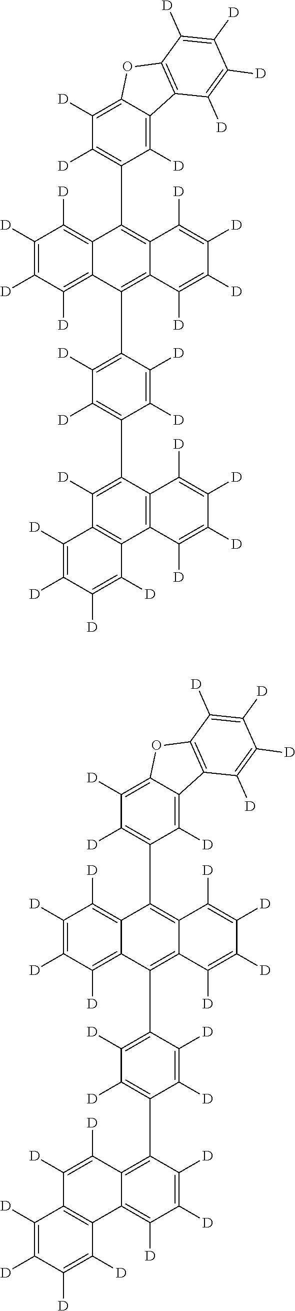 [SMILES: [2H]c1c([2H])c(-c2c([2H])c3c([2H])c([2H])c([2H])c([2H])c3c3c([2H])c([2H])c([2H])c([2H])c23)c([2H])c([2H])c1-c1c2c([2H])c([2H])c([2H])c([2H])c2c(-c2c([2H])c([2H])c3oc4c([2H])c([2H])c([2H])c([2H])c4c3c2[2H])c2c([2H])c([2H])c([2H])c([2H])c12 ; [2H]c1c([2H])c(-c2c([2H])c([2H])c([2H])c3c2c([2H])c([2H])c2c([2H])c([2H])c([2H])c([2H])c23)c([2H])c([2H])c1-c1c2c([2H])c([2H])c([2H])c([2H])c2c(-c2c([2H])c([2H])c3oc4c([2H])c([2H])c([2H])c([2H])c4c3c2[2H])c2c([2H])c([2H])c([2H])c([2H])c12]

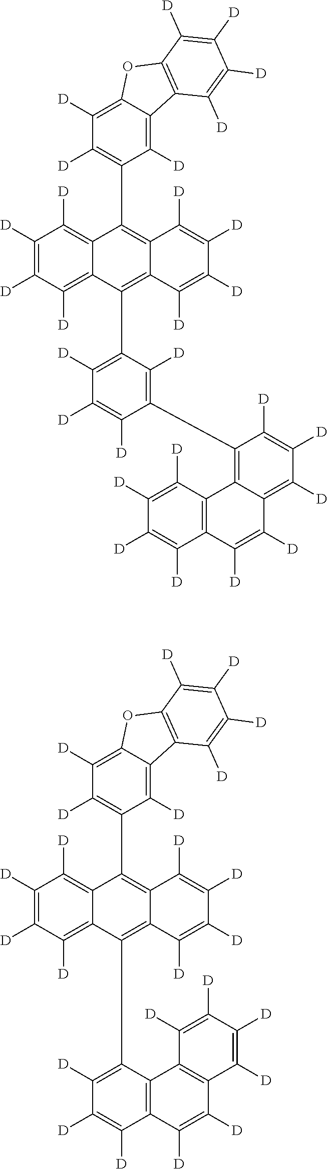 [SMILES: [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c(-c2c([2H])c([2H])c([2H])c3c([2H])c([2H])c4c([2H])c([2H])c([2H])c([2H])c4c23)c1[2H] ; [2H]c1c([2H])c([2H])c2c(oc3c([2H])c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c([2H])c32)c1[2H]]

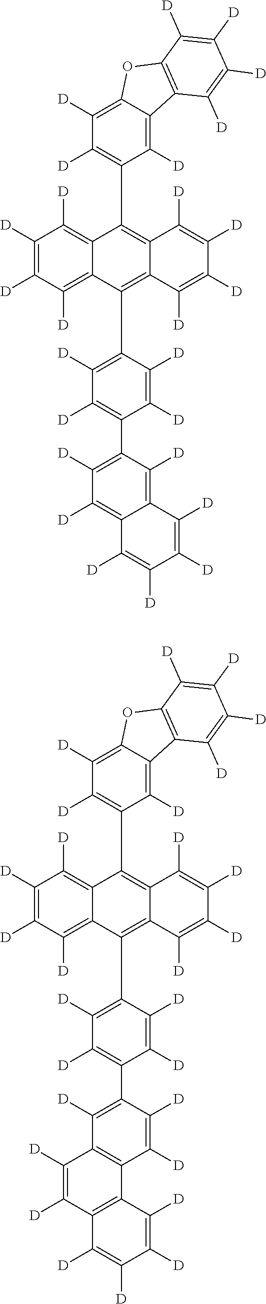 [SMILES: [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c([2H])c1-c1c([2H])c([2H])c2c([2H])c([2H])c([2H])c([2H])c2c1[2H] ; [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c([2H])c1-c1c([2H])c([2H])c2c(c1[2H])c([2H])c([2H])c1c([2H])c([2H])c([2H])c([2H])c12]

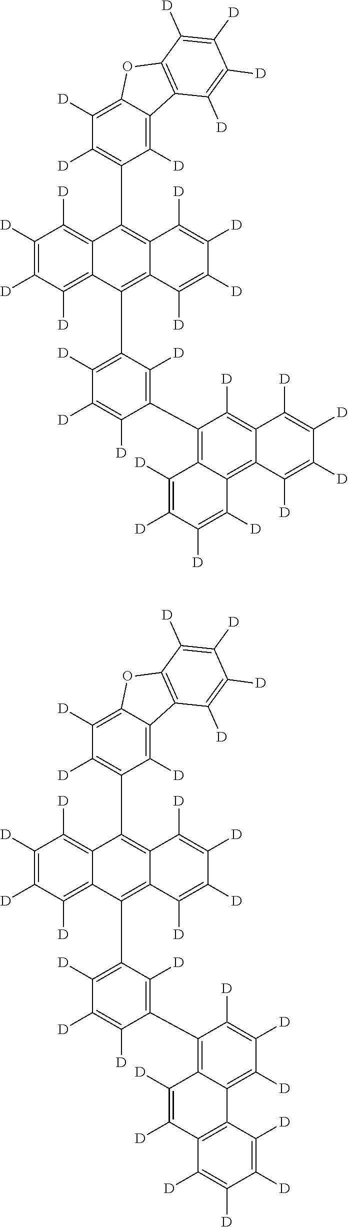 [SMILES: [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c(-c2c([2H])c3c([2H])c([2H])c([2H])c([2H])c3c3c([2H])c([2H])c([2H])c([2H])c23)c1[2H] ; [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c(-c2c([2H])c([2H])c([2H])c3c2c([2H])c([2H])c2c([2H])c([2H])c([2H])c([2H])c23)c1[2H]]

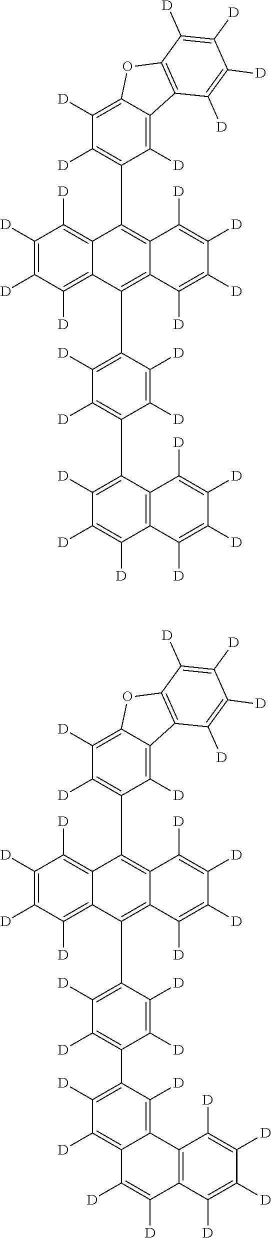 [SMILES: [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c([2H])c1-c1c([2H])c([2H])c([2H])c2c([2H])c([2H])c([2H])c([2H])c12 ; [2H]c1c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c([2H])c1-c1c([2H])c([2H])c2c([2H])c([2H])c3c([2H])c([2H])c([2H])c([2H])c3c2c1[2H]]

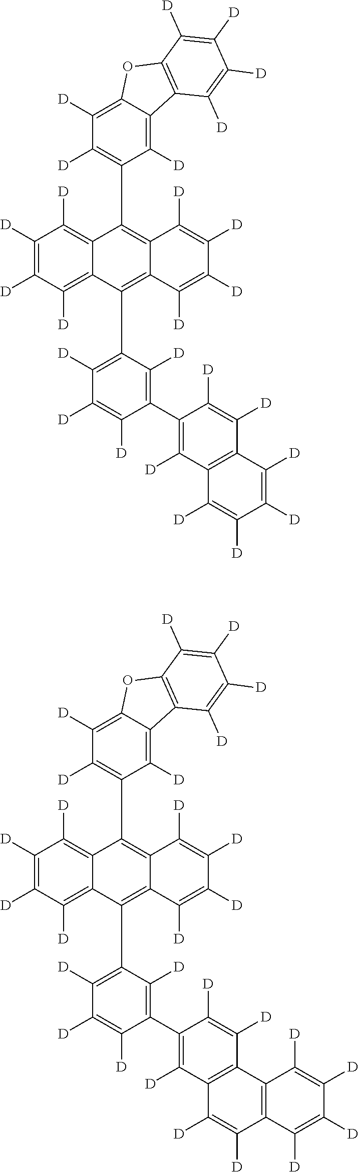 [SMILES: [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c1[2H] ; [2H]c1c([2H])c(-c2c([2H])c([2H])c3c(c2[2H])c([2H])c([2H])c2c([2H])c([2H])c([2H])c([2H])c23)c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c1[2H]]

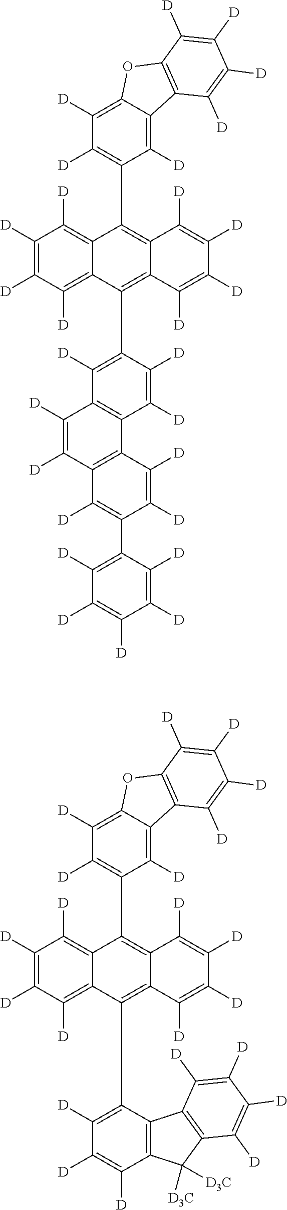 [SMILES: [2H]c1c([2H])c([2H])c(-c2c([2H])c([2H])c3c(c2[2H])c([2H])c([2H])c2c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c([2H])c23)c([2H])c1[2H] ; [2H]c1c([2H])c([2H])c2c(c1[2H])-c1c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c([2H])c([2H])c1C2(C([2H])([2H])[2H])C([2H])([2H])[2H]]

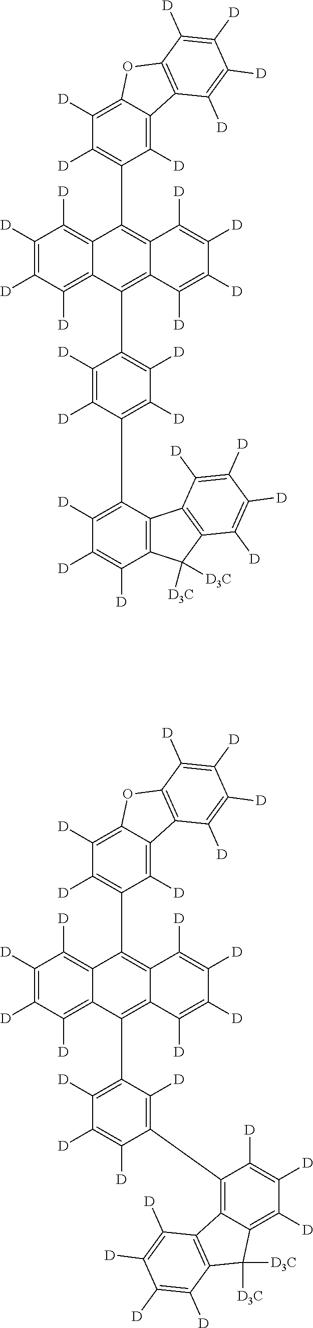

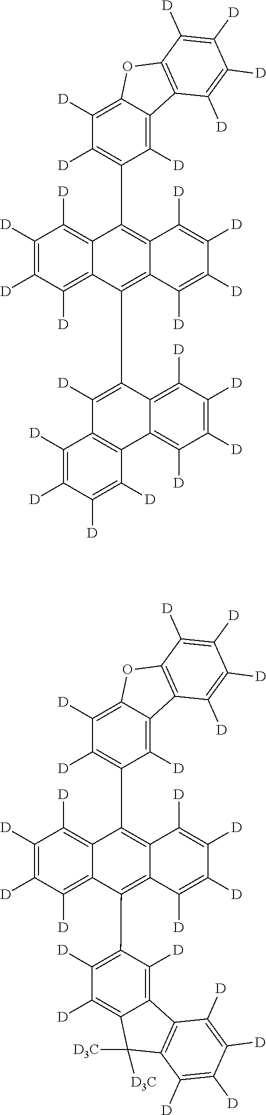 [SMILES: [2H]c1c([2H])c([2H])c2c(oc3c([2H])c([2H])c(-c4c5c([2H])c([2H])c([2H])c([2H])c5c(-c5c([2H])c6c([2H])c([2H])c([2H])c([2H])c6c6c([2H])c([2H])c([2H])c([2H])c56)c5c([2H])c([2H])c([2H])c([2H])c45)c([2H])c32)c1[2H] ; [2H]c1c([2H])c([2H])c2c(c1[2H])-c1c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c([2H])c1C2(C([2H])([2H])[2H])C([2H])([2H])[2H]]

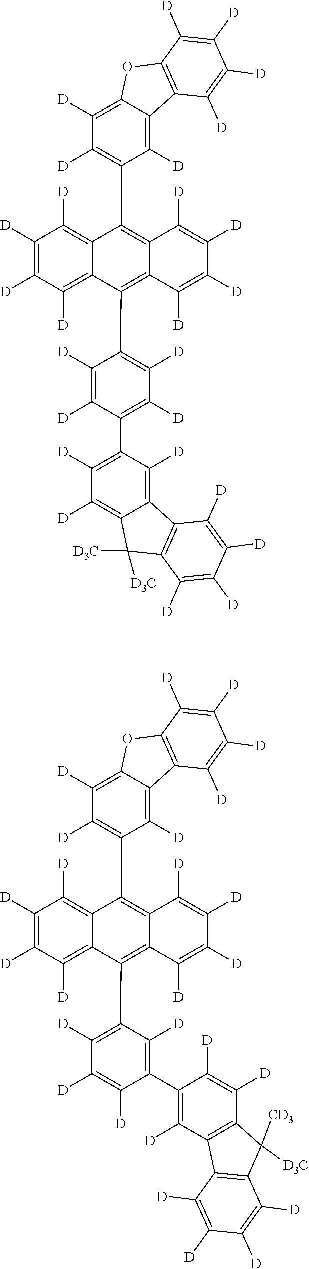

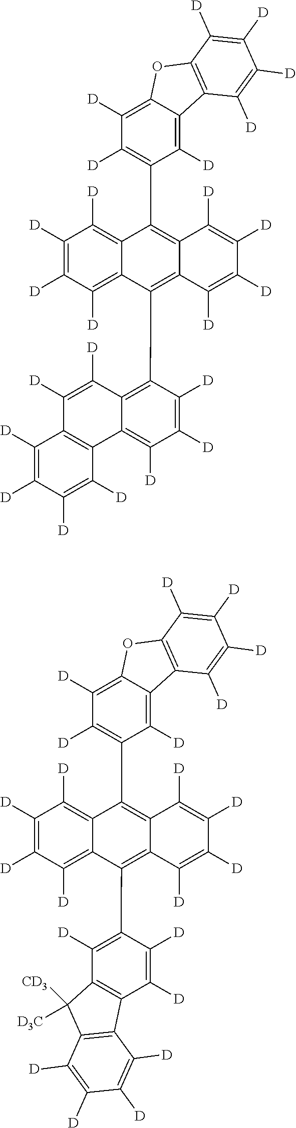 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c([2H])c32)c1[2H] ; [2H]c1c([2H])c([2H])c2c(c1[2H])-c1c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c1C2(C([2H])([2H])[2H])C([2H])([2H])[2H]]

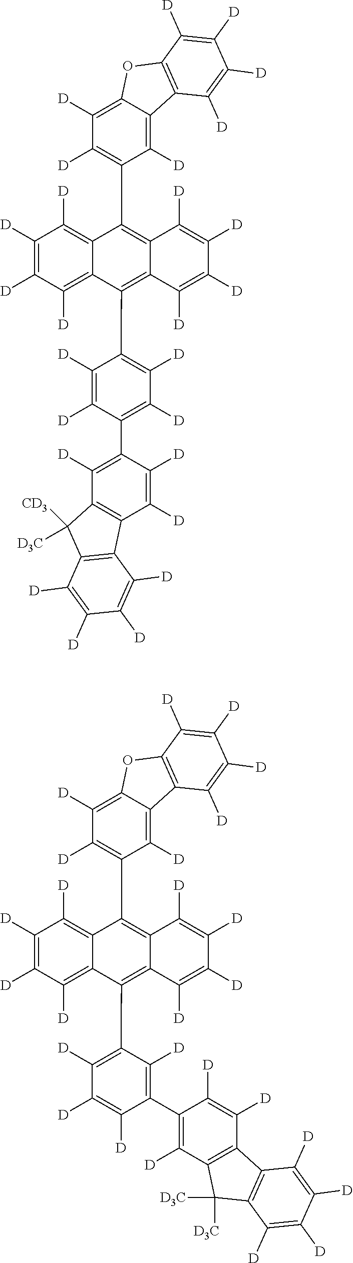

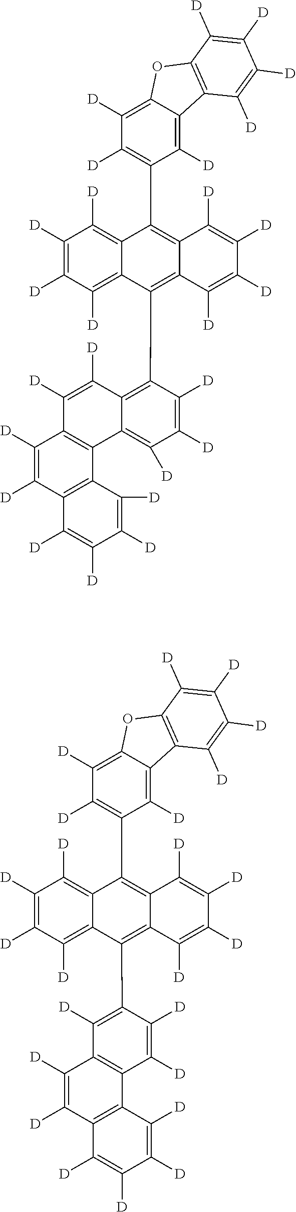 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c5c([2H])c([2H])c5c([2H])c([2H])c7c([2H])c([2H])c([2H])c([2H])c7c56)c5c([2H])c([2H])c([2H])c([2H])c45)c([2H])c32)c1[2H] ; [2H]c1c([2H])c([2H])c2c(oc3c([2H])c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c([2H])c32)c1[2H]]

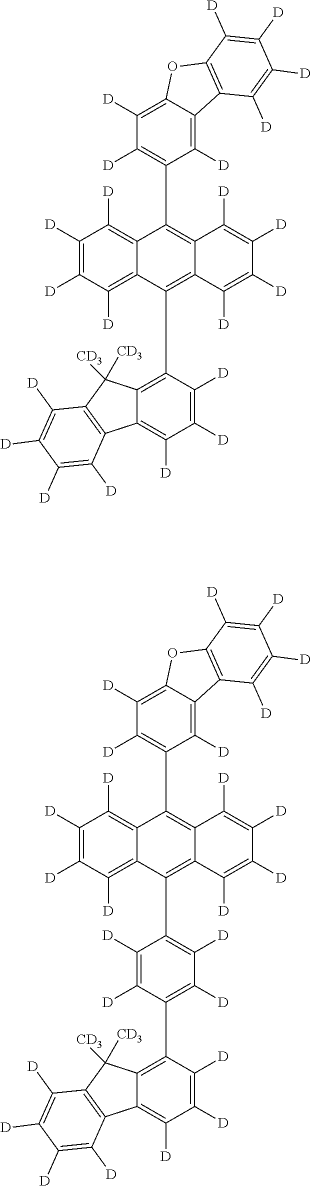 [SMILES: [2H]c1c([2H])c([2H])c2c(c1[2H])-c1c([2H])c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c1C2(C([2H])([2H])[2H])C([2H])([2H])[2H] ; [2H]c1c([2H])c([2H])c2c(c1[2H])-c1c([2H])c([2H])c([2H])c(-c3c([2H])c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c3[2H])c1C2(C([2H])([2H])[2H])C([2H])([2H])[2H]]

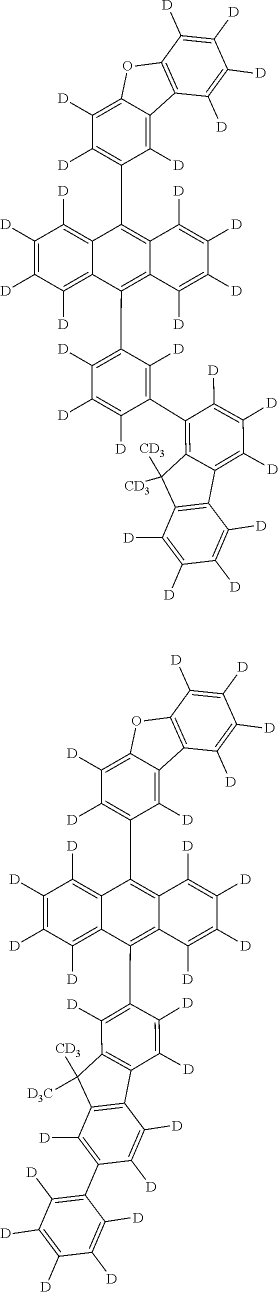

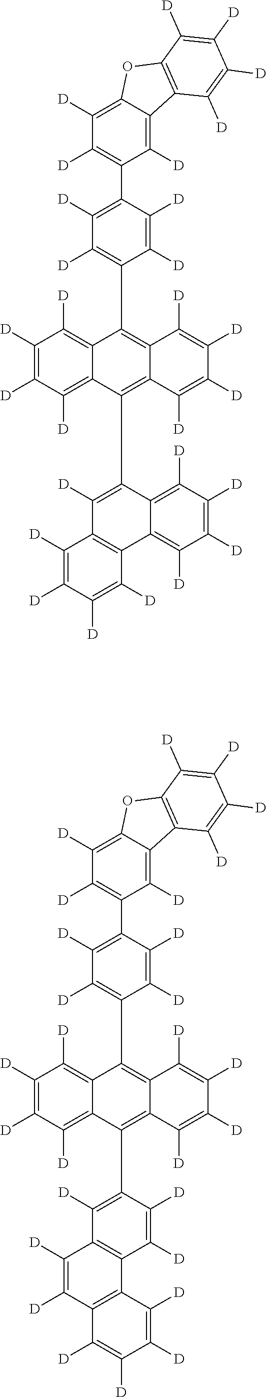 [SMILES: [2H]c1c([2H])c(-c2c3c([2H])c([2H])c([2H])c([2H])c3c(-c3c([2H])c4c([2H])c([2H])c([2H])c([2H])c4c4c([2H])c([2H])c([2H])c([2H])c34)c3c([2H])c([2H])c([2H])c([2H])c23)c([2H])c([2H])c1-c1c([2H])c([2H])c2oc3c([2H])c([2H])c([2H])c([2H])c3c2c1[2H] ; [2H]c1c([2H])c(-c2c3c([2H])c([2H])c([2H])c([2H])c3c(-c3c([2H])c([2H])c4c(c3[2H])c([2H])c([2H])c3c([2H])c([2H])c([2H])c([2H])c34)c3c([2H])c([2H])c([2H])c([2H])c23)c([2H])c([2H])c1-c1c([2H])c([2H])c2oc3c([2H])c([2H])c([2H])c([2H])c3c2c1[2H]]

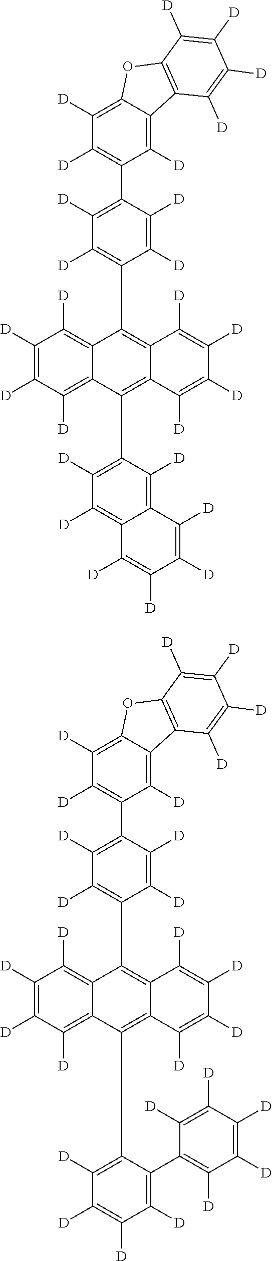

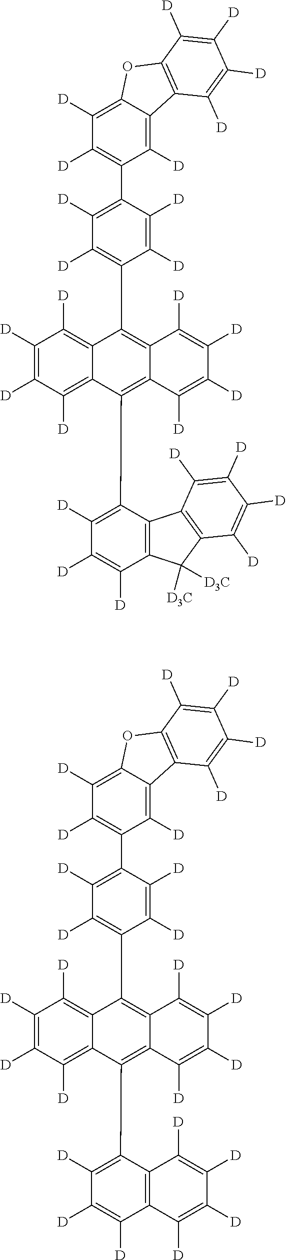 [SMILES: [2H]c1c([2H])c([2H])c2c(c1[2H])-c1c(-c3c4c([2H])c([2H])c([2H])c([2H])c4c(-c4c([2H])c([2H])c(-c5c([2H])c([2H])c6oc7c([2H])c([2H])c([2H])c([2H])c7c6c5[2H])c([2H])c4[2H])c4c([2H])c([2H])c([2H])c([2H])c34)c([2H])c([2H])c([2H])c1C2(C([2H])([2H])[2H])C([2H])([2H])[2H] ; [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c([2H])c1-c1c([2H])c([2H])c2oc3c([2H])c([2H])c([2H])c([2H])c3c2c1[2H]]

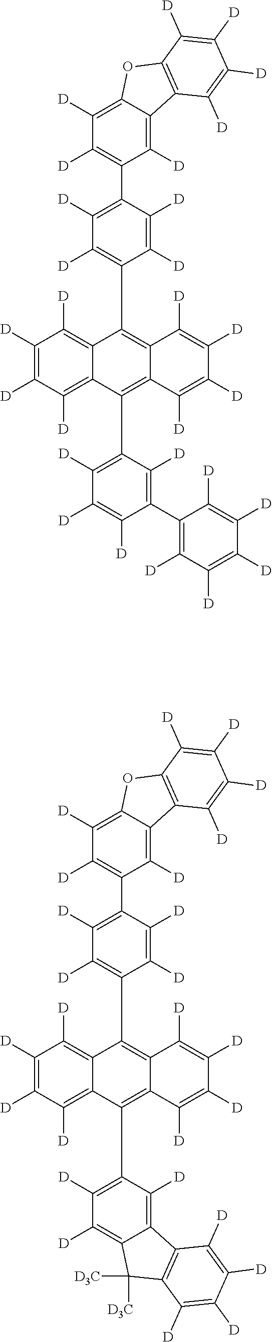 [SMILES: [2H]c1c([2H])c([2H])c(-c2c([2H])c([2H])c([2H])c(-c3c4c([2H])c([2H])c([2H])c([2H])c4c(-c4c([2H])c([2H])c(-c5c([2H])c([2H])c6oc7c([2H])c([2H])c([2H])c([2H])c7c6c5[2H])c([2H])c4[2H])c4c([2H])c([2H])c([2H])c([2H])c34)c2[2H])c([2H])c1[2H] ; [2H]c1c([2H])c([2H])c2c(c1[2H])-c1c([2H])c(-c3c4c([2H])c([2H])c([2H])c([2H])c4c(-c4c([2H])c([2H])c(-c5c([2H])c([2H])c6oc7c([2H])c([2H])c([2H])c([2H])c7c6c5[2H])c([2H])c4[2H])c4c([2H])c([2H])c([2H])c([2H])c34)c([2H])c([2H])c1C2(C([2H])([2H])[2H])C([2H])([2H])[2H]]

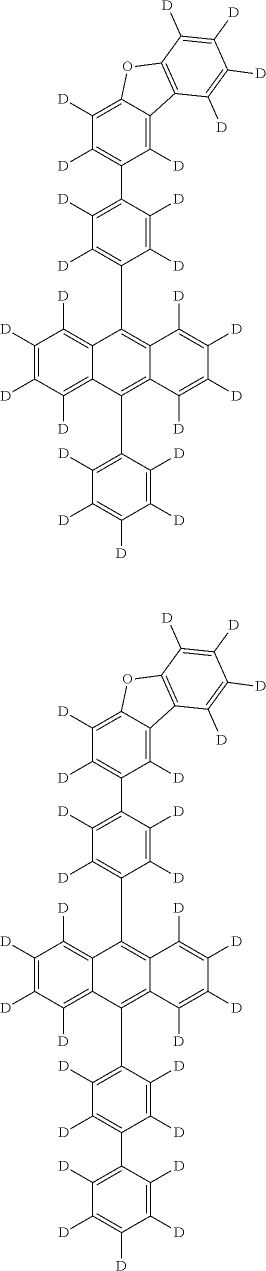 [SMILES: [2H]c1c([2H])c([2H])c(-c2c3c([2H])c([2H])c([2H])c([2H])c3c(-c3c([2H])c([2H])c(-c4c([2H])c([2H])c5oc6c([2H])c([2H])c([2H])c([2H])c6c5c4[2H])c([2H])c3[2H])c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4c([2H])c([2H])c(-c5c([2H])c([2H])c6oc7c([2H])c([2H])c([2H])c([2H])c7c6c5[2H])c([2H])c4[2H])c4c([2H])c([2H])c([2H])c([2H])c34)c([2H])c2[2H])c([2H])c1[2H]]

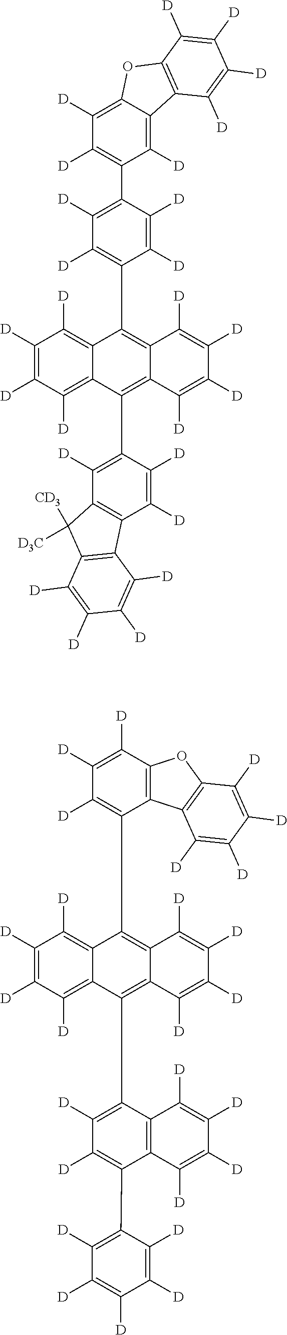 [SMILES: [2H]c1c([2H])c([2H])c2c(c1[2H])-c1c([2H])c([2H])c(-c3c4c([2H])c([2H])c([2H])c([2H])c4c(-c4c([2H])c([2H])c(-c5c([2H])c([2H])c6oc7c([2H])c([2H])c([2H])c([2H])c7c6c5[2H])c([2H])c4[2H])c4c([2H])c([2H])c([2H])c([2H])c34)c([2H])c1C2(C([2H])([2H])[2H])C([2H])([2H])[2H] ; [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c3c([2H])c([2H])c([2H])c([2H])c23)c([2H])c1[2H]]

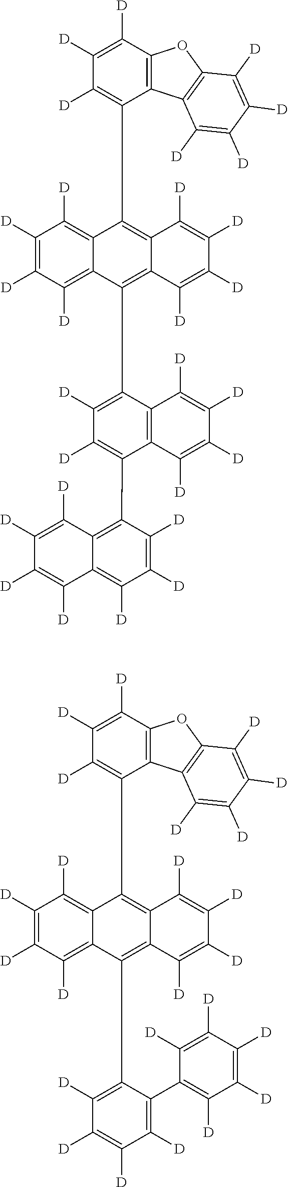 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c(-c6c([2H])c([2H])c([2H])c7c([2H])c([2H])c([2H])c([2H])c67)c6c([2H])c([2H])c([2H])c([2H])c56)c5c([2H])c([2H])c([2H])c([2H])c45)c32)c1[2H] ; [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c1[2H]]

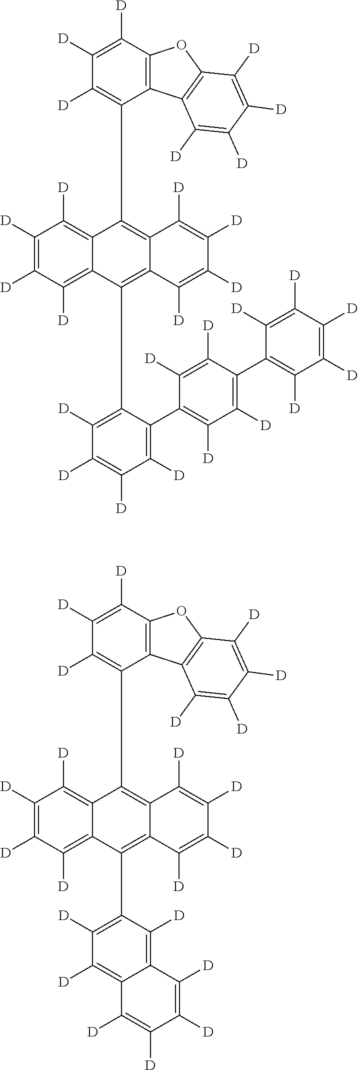 [SMILES: [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2[2H])c([2H])c1[2H] ; [2H]c1c([2H])c([2H])c2c(oc3c([2H])c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c32)c1[2H]]

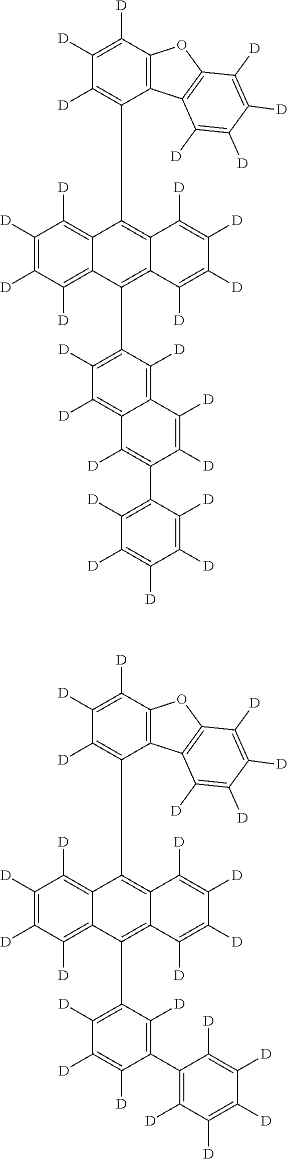 [SMILES: [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c([2H])c3c2[2H])c([2H])c1[2H] ; [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c2[2H])c([2H])c1[2H]]

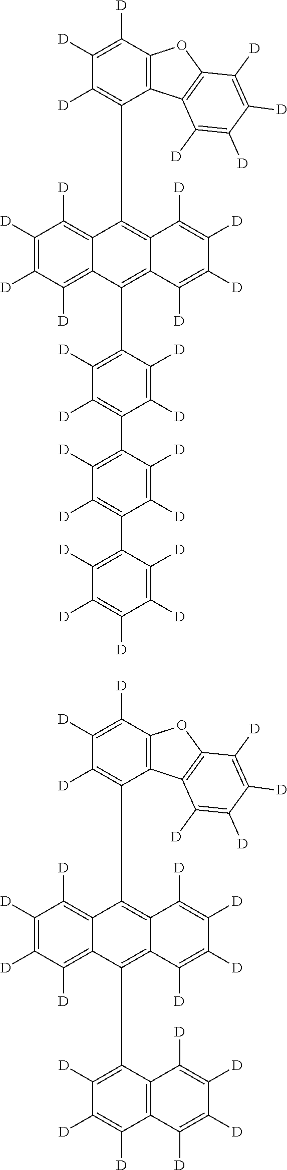

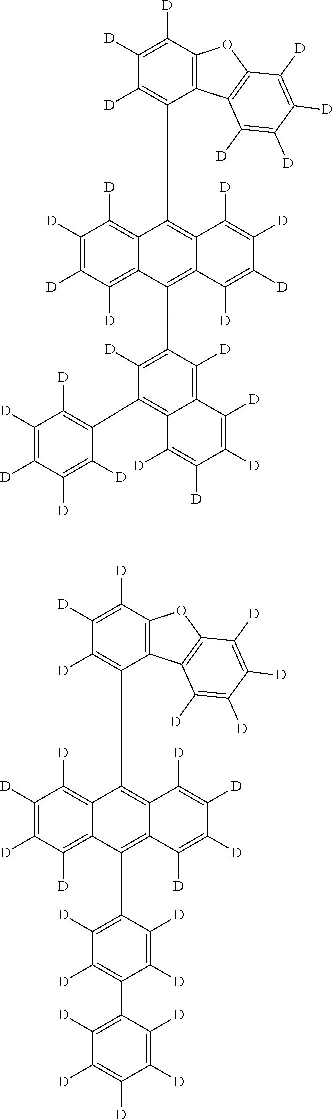 [SMILES: [2H]c1c([2H])c([2H])c(-c2c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2[2H])c([2H])c1[2H]]

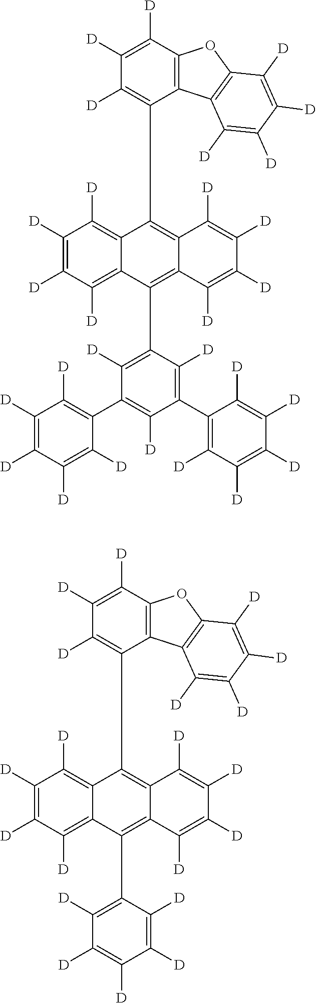 [SMILES: [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c2[2H])c([2H])c1[2H] ; [2H]c1c([2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c1[2H]]

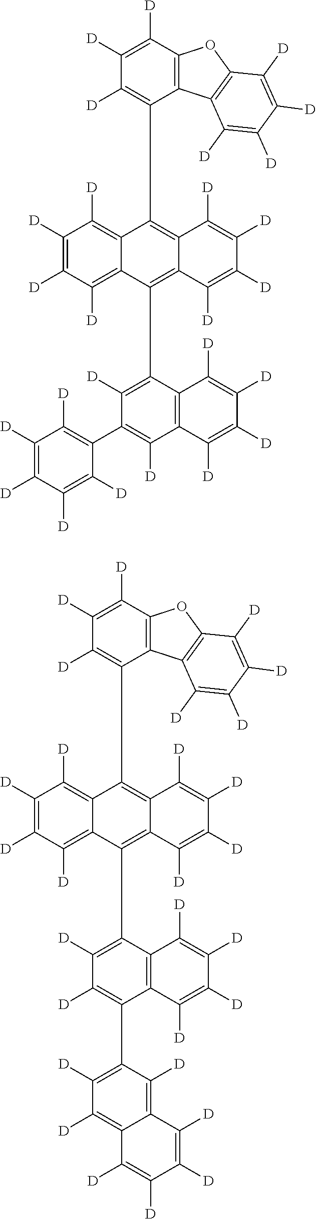 [SMILES: [2H]c1c([2H])c([2H])c(-c2c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c3c([2H])c([2H])c([2H])c([2H])c3c2[2H])c([2H])c1[2H] ; [2H]c1c([2H])c([2H])c2c(oc3c([2H])c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c32)c1[2H]]

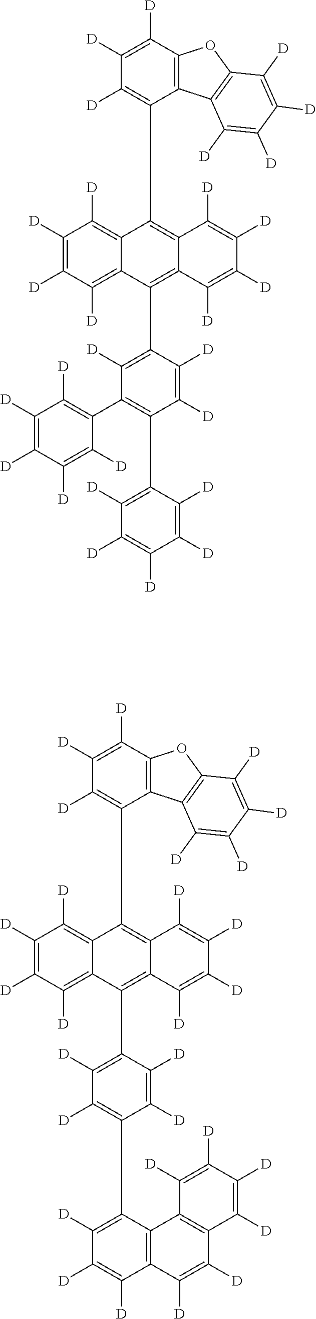 [SMILES: [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c2-c2c([2H])c([2H])c([2H])c([2H])c2[2H])c([2H])c1[2H] ; [2H]c1c([2H])c(-c2c([2H])c([2H])c([2H])c3c([2H])c([2H])c4c([2H])c([2H])c([2H])c([2H])c4c23)c([2H])c([2H])c1-c1c2c([2H])c([2H])c([2H])c([2H])c2c(-c2c([2H])c([2H])c([2H])c3oc4c([2H])c([2H])c([2H])c([2H])c4c23)c2c([2H])c([2H])c([2H])c([2H])c12]

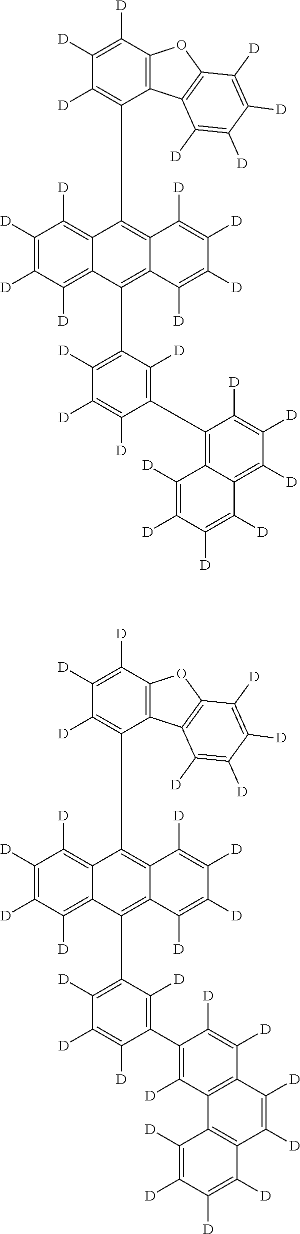 [SMILES: [2H]c1c([2H])c(-c2c([2H])c([2H])c([2H])c3c([2H])c([2H])c([2H])c([2H])c23)c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H] ; [2H]c1c([2H])c(-c2c([2H])c([2H])c3c([2H])c([2H])c4c([2H])c([2H])c([2H])c([2H])c4c3c2[2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H]]

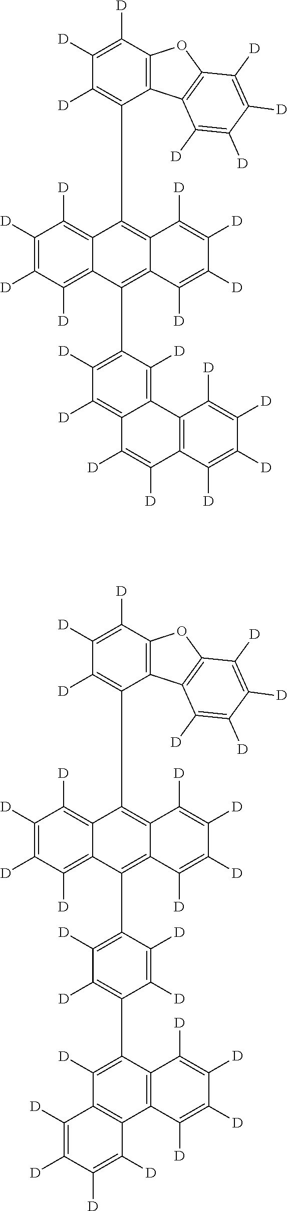 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c1[2H] ; [2H]c1c([2H])c(-c2c([2H])c3c([2H])c([2H])c([2H])c([2H])c3c3c([2H])c([2H])c([2H])c([2H])c23)c([2H])c([2H])c1-c1c2c([2H])c([2H])c([2H])c([2H])c2c(-c2c([2H])c([2H])c([2H])c3oc4c([2H])c([2H])c([2H])c([2H])c4c23)c2c([2H])c([2H])c([2H])c([2H])c12]

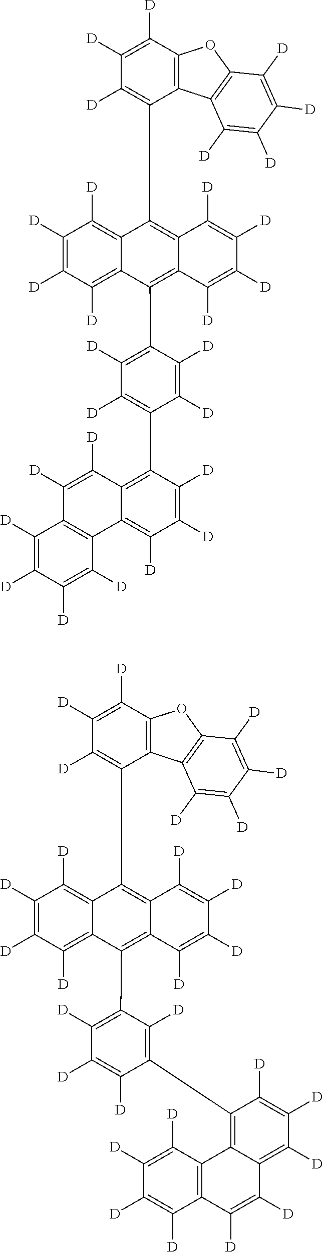 [SMILES: [2H]c1c([2H])c(-c2c([2H])c([2H])c([2H])c3c2c([2H])c([2H])c2c([2H])c([2H])c([2H])c([2H])c23)c([2H])c([2H])c1-c1c2c([2H])c([2H])c([2H])c([2H])c2c(-c2c([2H])c([2H])c([2H])c3oc4c([2H])c([2H])c([2H])c([2H])c4c23)c2c([2H])c([2H])c([2H])c([2H])c12 ; [2H]c1c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c(-c2c([2H])c([2H])c([2H])c3c([2H])c([2H])c4c([2H])c([2H])c([2H])c([2H])c4c23)c1[2H]]

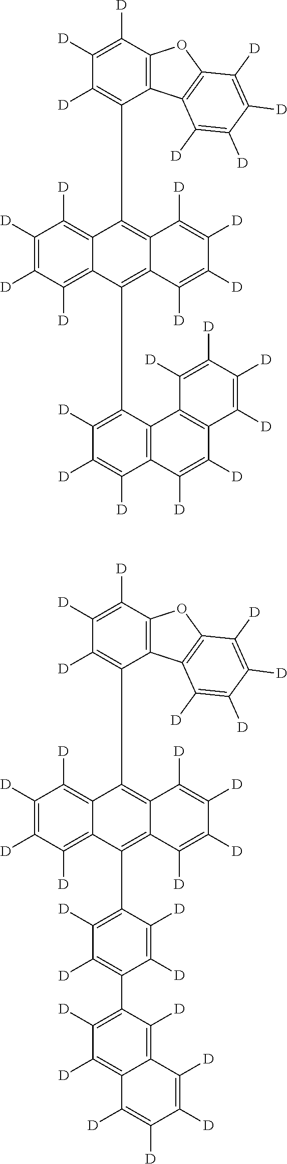 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c1[2H] ; [2H]c1c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c([2H])c1-c1c([2H])c([2H])c2c([2H])c([2H])c([2H])c([2H])c2c1[2H]]

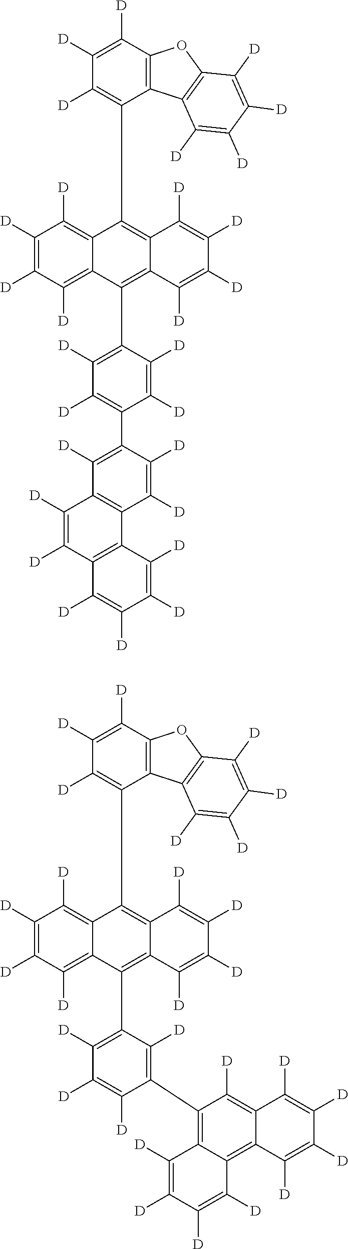

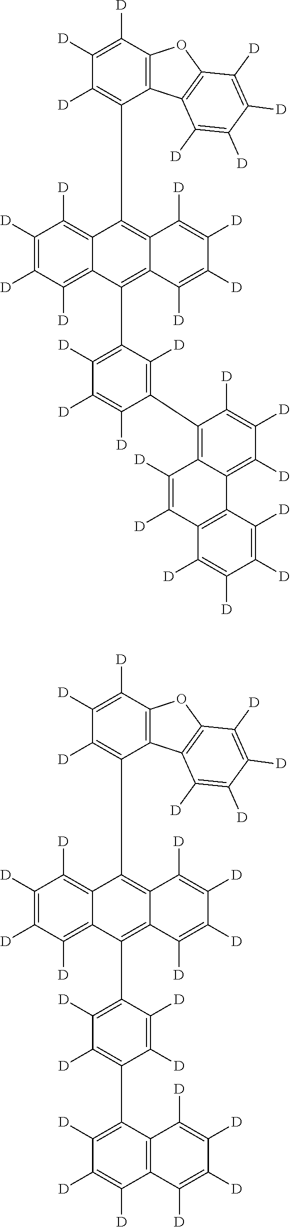 [SMILES: [2H]c1c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c(-c2c([2H])c([2H])c([2H])c3c2c([2H])c([2H])c2c([2H])c([2H])c([2H])c([2H])c23)c1[2H] ; [2H]c1c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c([2H])c1-c1c([2H])c([2H])c([2H])c2c([2H])c([2H])c([2H])c([2H])c12]

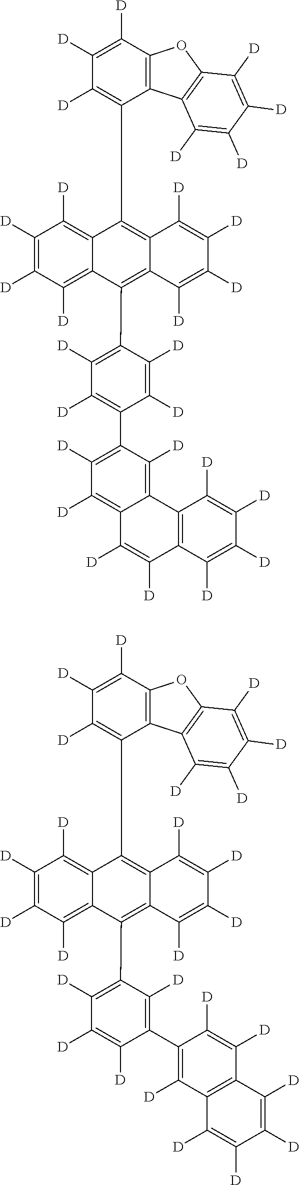 [SMILES: [2H]c1c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c([2H])c([2H])c1-c1c([2H])c([2H])c2c([2H])c([2H])c3c([2H])c([2H])c([2H])c([2H])c3c2c1[2H] ; [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H]]

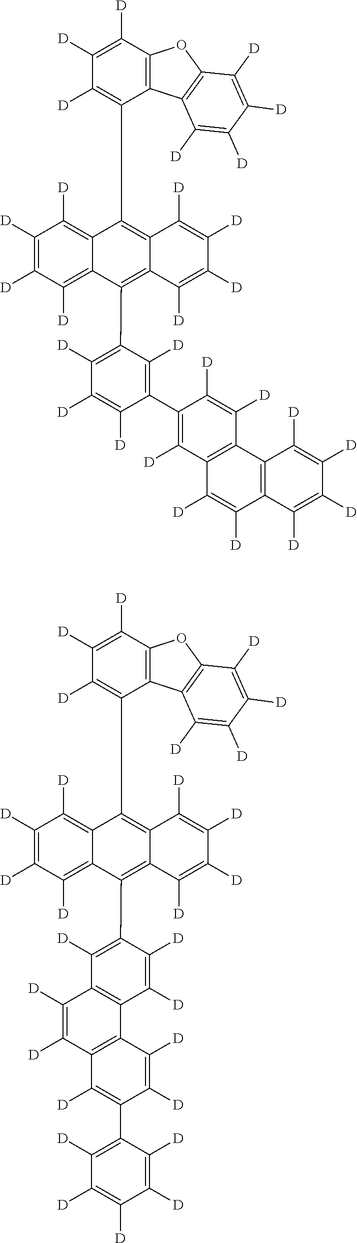 [SMILES: [2H]c1c([2H])c(-c2c([2H])c([2H])c3c(c2[2H])c([2H])c([2H])c2c([2H])c([2H])c([2H])c([2H])c23)c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H] ; [2H]c1c([2H])c([2H])c(-c2c([2H])c([2H])c3c(c2[2H])c([2H])c([2H])c2c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c([2H])c23)c([2H])c1[2H]]

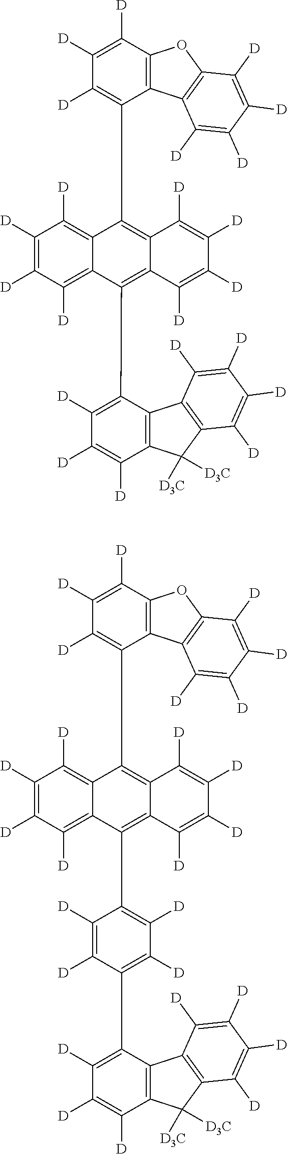 [SMILES: [2H]c1c([2H])c([2H])c2c(c1[2H])-c1c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c([2H])c([2H])c1C2(C([2H])([2H])[2H])C([2H])([2H])[2H] ; [2H]c1c([2H])c([2H])c2c(c1[2H])-c1c(-c3c([2H])c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c3[2H])c([2H])c([2H])c([2H])c1C2(C([2H])([2H])[2H])C([2H])([2H])[2H]]

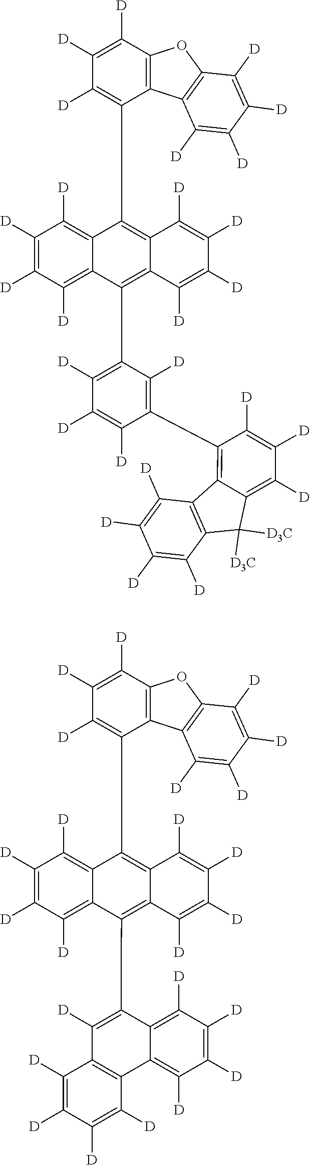 [SMILES: [2H]c1c([2H])c(-c2c([2H])c([2H])c([2H])c3c2-c2c([2H])c([2H])c([2H])c([2H])c2C3(C([2H])([2H])[2H])C([2H])([2H])[2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H] ; [2H]c1c([2H])c([2H])c2c(oc3c([2H])c([2H])c([2H])c(-c4c5c([2H])c([2H])c([2H])c([2H])c5c(-c5c([2H])c6c([2H])c([2H])c([2H])c([2H])c6c6c([2H])c([2H])c([2H])c([2H])c56)c5c([2H])c([2H])c([2H])c([2H])c45)c32)c1[2H]]

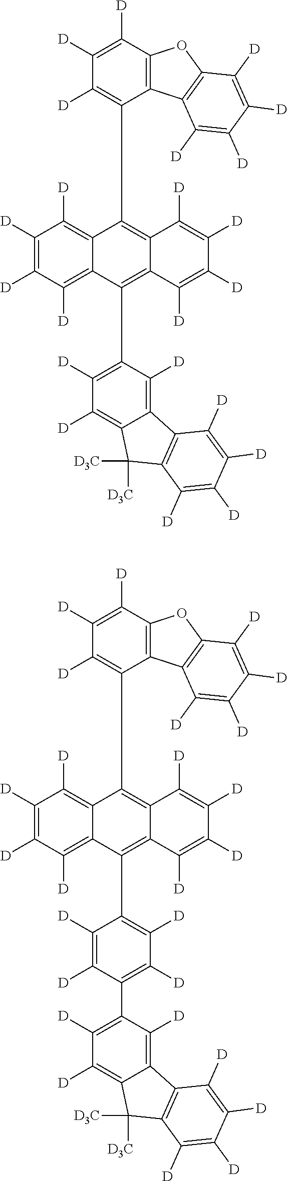 [SMILES: [2H]c1c([2H])c([2H])c2c(c1[2H])-c1c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c([2H])c1C2(C([2H])([2H])[2H])C([2H])([2H])[2H] ; [2H]c1c([2H])c([2H])c2c(c1[2H])-c1c([2H])c(-c3c([2H])c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c3[2H])c([2H])c([2H])c1C2(C([2H])([2H])[2H])C([2H])([2H])[2H]]

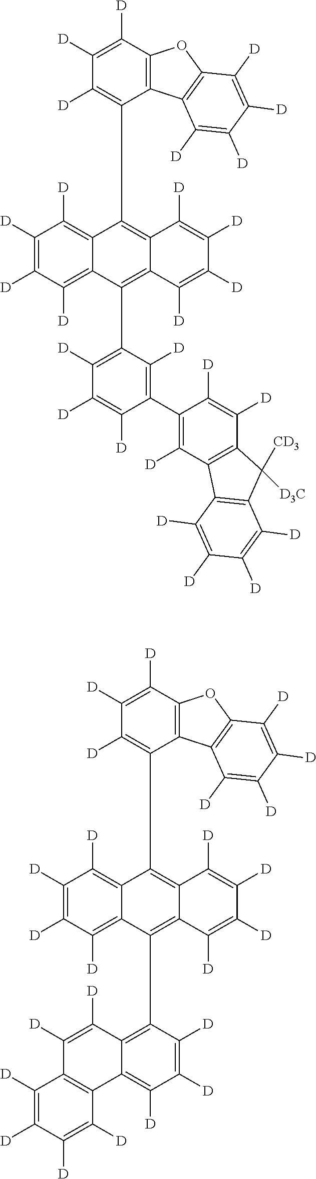 [SMILES: [2H]c1c([2H])c(-c2c([2H])c([2H])c3c(c2[2H])-c2c([2H])c([2H])c([2H])c([2H])c2C3(C([2H])([2H])[2H])C([2H])([2H])[2H])c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H] ; [2H]c1c([2H])c([2H])c2c(oc3c([2H])c([2H])c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c1[2H]]

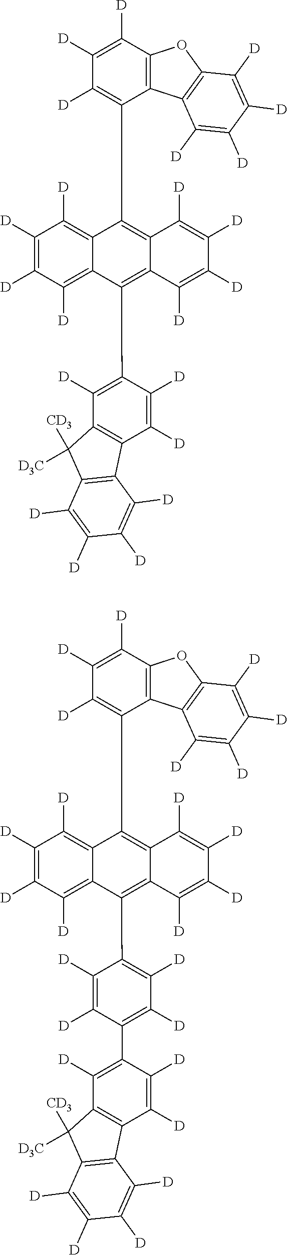 [SMILES: [2H]c1c([2H])c([2H])c2c(c1[2H])-c1c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c([2H])c1C2(C([2H])([2H])[2H])C([2H])([2H])[2H] ; [2H]c1c([2H])c([2H])c2c(c1[2H])-c1c([2H])c([2H])c(-c3c([2H])c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c3[2H])c([2H])c1C2(C([2H])([2H])[2H])C([2H])([2H])[2H]]

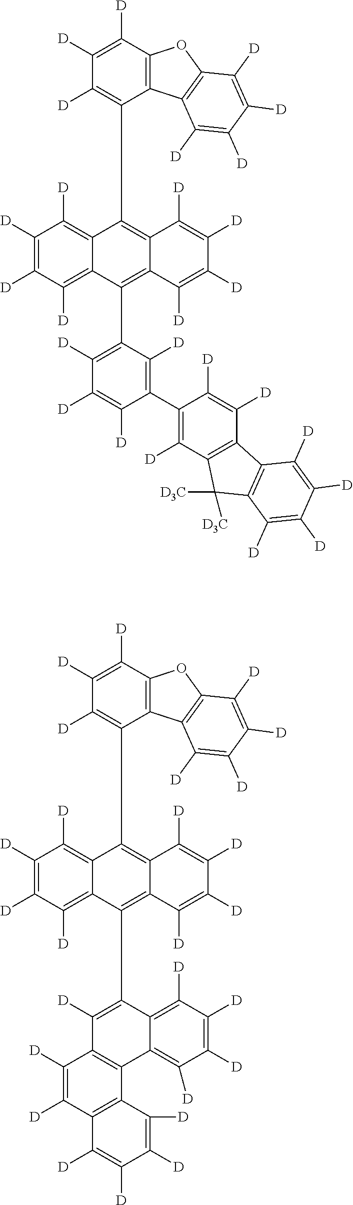 [SMILES: [2H]c1c([2H])c(-c2c([2H])c([2H])c3c(c2[2H])C(C([2H])([2H])[2H])(C([2H])([2H])[2H])c2c([2H])c([2H])c([2H])c([2H])c2-3)c([2H])c(-c2c3c([2H])c([2H])c([2H])c([2H])c3c(-c3c([2H])c([2H])c([2H])c4oc5c([2H])c([2H])c([2H])c([2H])c5c34)c3c([2H])c([2H])c([2H])c([2H])c23)c1[2H] ; [2H]c1c([2H])c([2H])c2c(oc3c([2H])c([2H])c([2H])c(-c4c5c([2H])c([2H])c([2H])c([2H])c5c(-c5c([2H])c6c([2H])c([2H])c7c([2H])c([2H])c([2H])c([2H])c7c6c6c([2H])c([2H])c([2H])c([2H])c56)c5c([2H])c([2H])c([2H])c([2H])c45)c32)c1[2H]]

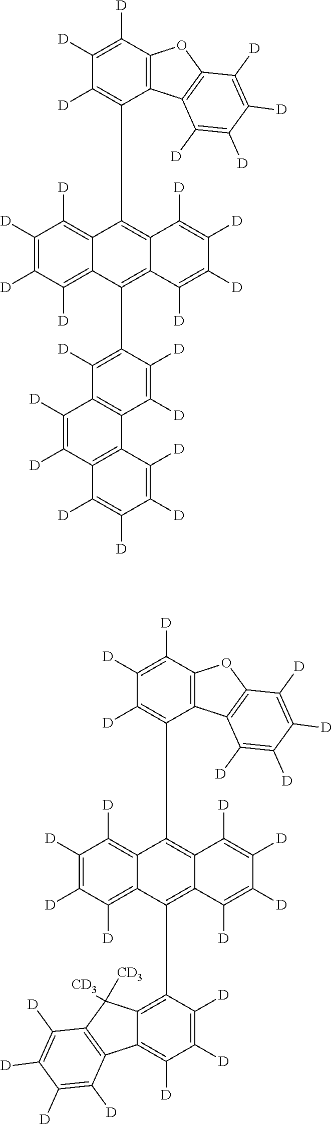 [SMILES: [2H]c1c([2H])c([2H])c2c(oc3c([2H])c([2H])c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c1[2H] ; [2H]c1c([2H])c([2H])c2c(c1[2H])-c1c([2H])c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c1C2(C([2H])([2H])[2H])C([2H])([2H])[2H]]

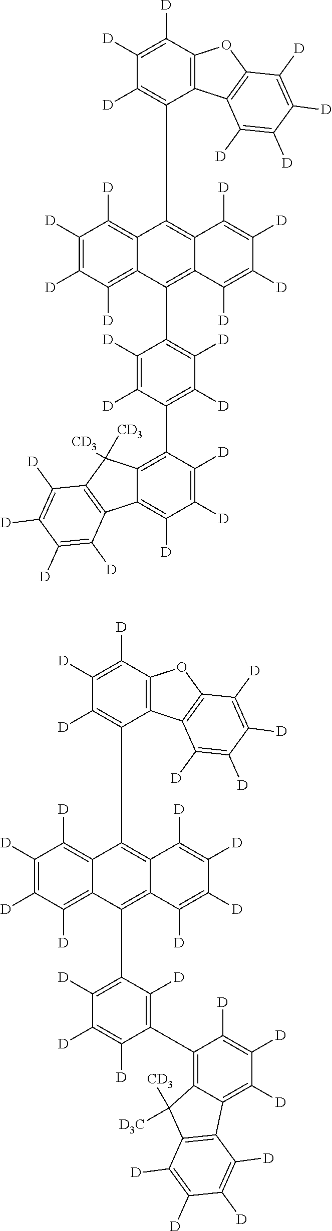

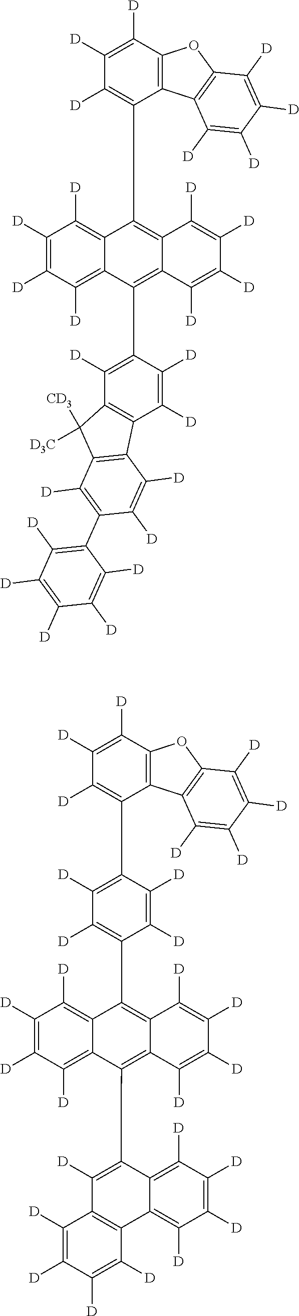

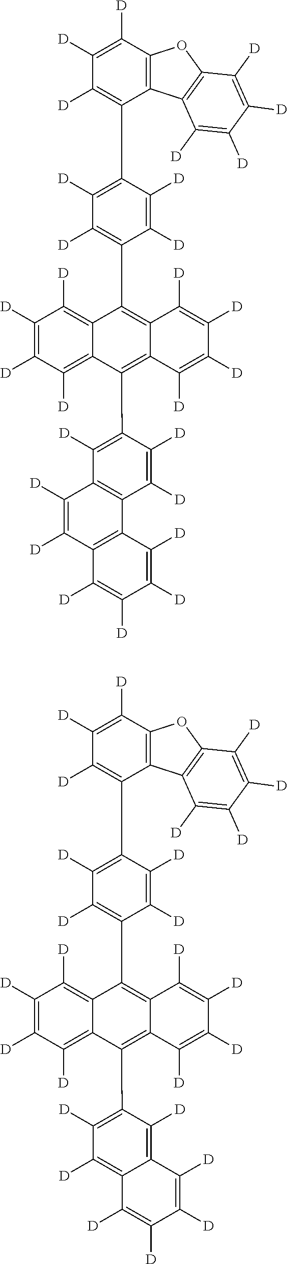 [SMILES: [2H]c1c([2H])c(-c2c([2H])c([2H])c([2H])c3oc4c([2H])c([2H])c([2H])c([2H])c4c23)c([2H])c([2H])c1-c1c2c([2H])c([2H])c([2H])c([2H])c2c(-c2c([2H])c([2H])c3c(c2[2H])c([2H])c([2H])c2c([2H])c([2H])c([2H])c([2H])c23)c2c([2H])c([2H])c([2H])c([2H])c12 ; [2H]c1c([2H])c(-c2c([2H])c([2H])c([2H])c3oc4c([2H])c([2H])c([2H])c([2H])c4c23)c([2H])c([2H])c1-c1c2c([2H])c([2H])c([2H])c([2H])c2c(-c2c([2H])c([2H])c3c([2H])c([2H])c([2H])c([2H])c3c2[2H])c2c([2H])c([2H])c([2H])c([2H])c12]

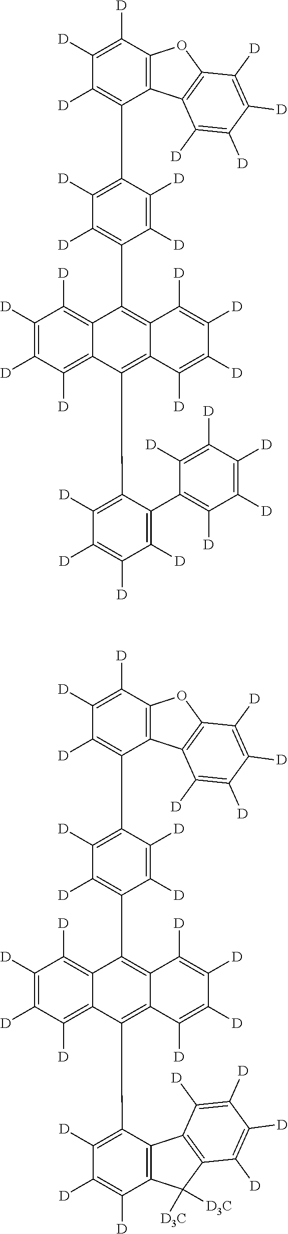

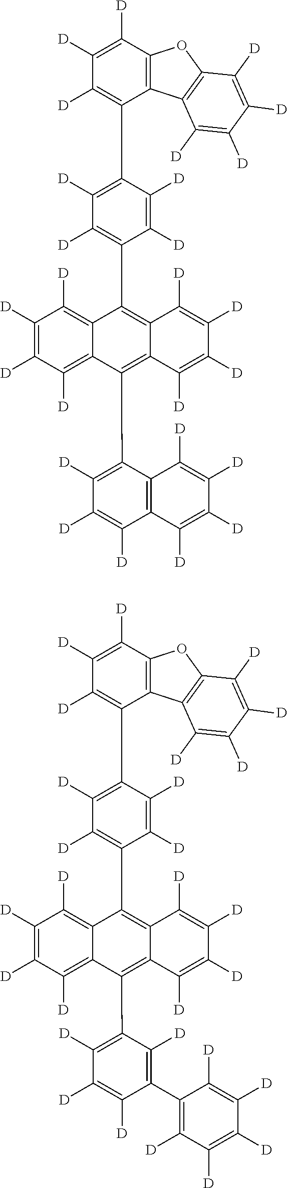

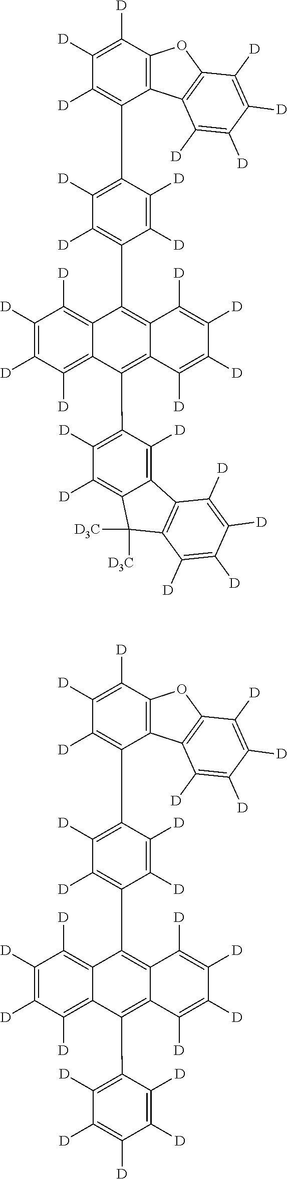 [SMILES: [2H]c1c([2H])c([2H])c2c(c1[2H])-c1c([2H])c(-c3c4c([2H])c([2H])c([2H])c([2H])c4c(-c4c([2H])c([2H])c(-c5c([2H])c([2H])c([2H])c6oc7c([2H])c([2H])c([2H])c([2H])c7c56)c([2H])c4[2H])c4c([2H])c([2H])c([2H])c([2H])c34)c([2H])c([2H])c1C2(C([2H])([2H])[2H])C([2H])([2H])[2H] ; [2H]c1c([2H])c([2H])c(-c2c3c([2H])c([2H])c([2H])c([2H])c3c(-c3c([2H])c([2H])c(-c4c([2H])c([2H])c([2H])c5oc6c([2H])c([2H])c([2H])c([2H])c6c45)c([2H])c3[2H])c3c([2H])c([2H])c([2H])c([2H])c23)c([2H])c1[2H]]

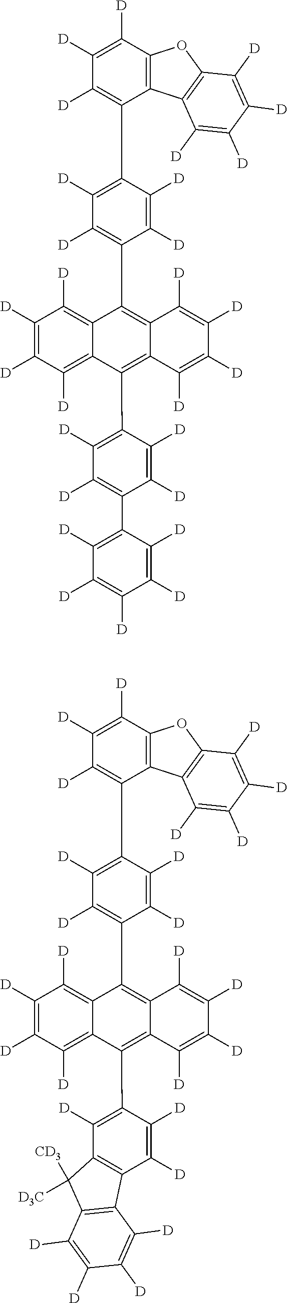

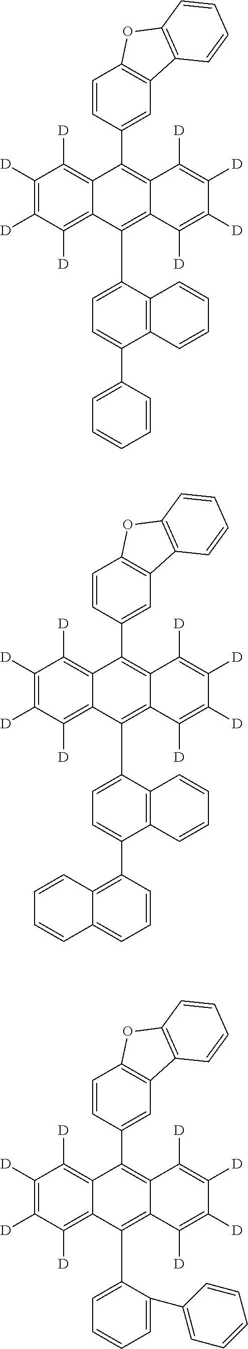 [SMILES: [2H]c1c([2H])c([2H])c2c(-c3ccc(-c4ccccc4)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc(-c4cccc5ccccc45)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccccc3-c3ccccc3)c3c([2H])c([2H])c([2H])c([2H])c3c(-c3ccc4oc5ccccc5c4c3)c2c1[2H]]

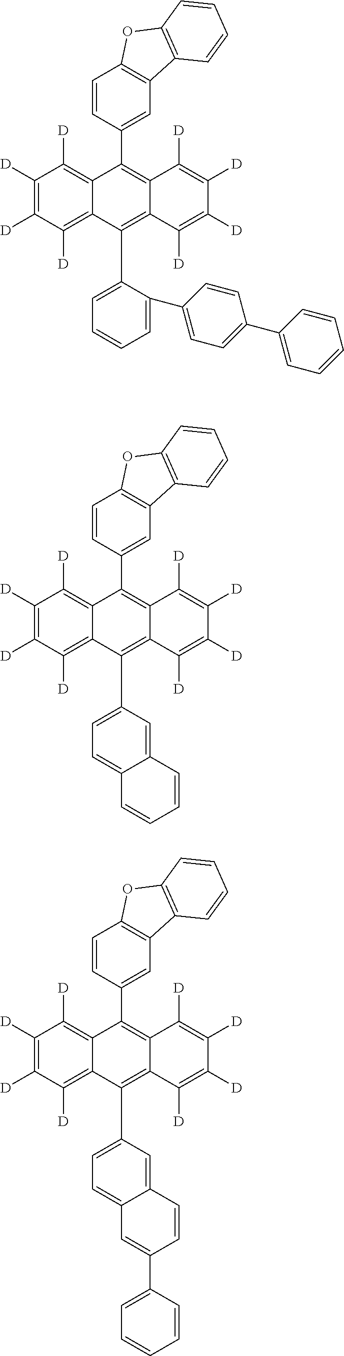

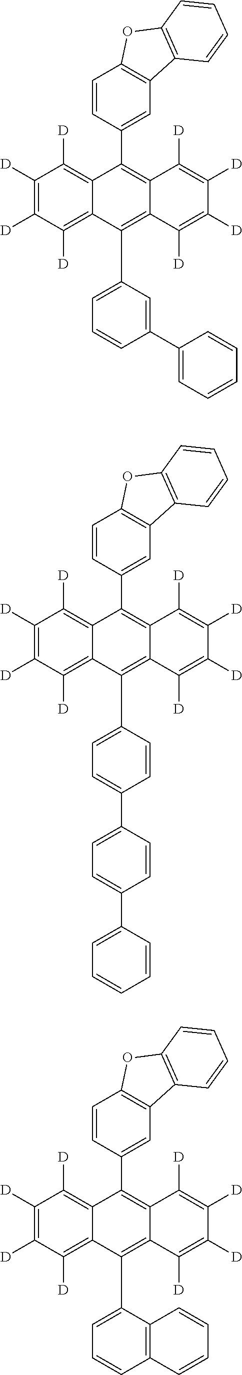 [SMILES: [2H]c1c([2H])c([2H])c2c(-c3ccc4oc5ccccc5c4c3)c3c([2H])c([2H])c([2H])c([2H])c3c(-c3cccc(-c4ccccc4)c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccc(-c5ccccc5)cc4)cc3)c2c1[2H] ; [2H]c1c([2H])c([2H])c2c(-c3cccc4ccccc34)c3c([2H])c([2H])c([2H])c([2H])c3c(-c3ccc4oc5ccccc5c4c3)c2c1[2H]]

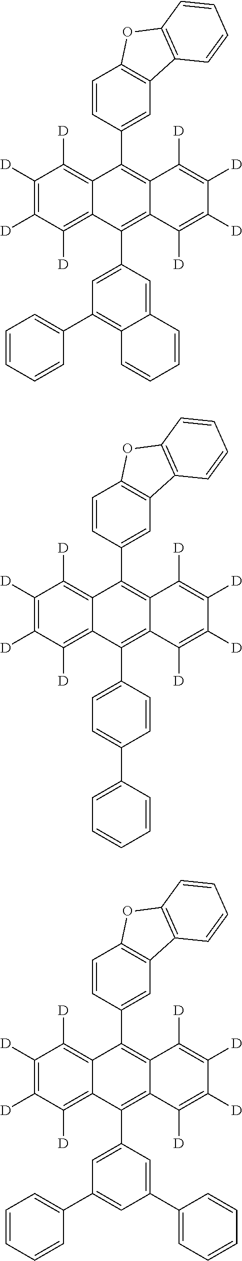 [SMILES: [2H]c1c([2H])c([2H])c2c(-c3ccc4oc5ccccc5c4c3)c3c([2H])c([2H])c([2H])c([2H])c3c(-c3cc(-c4ccccc4)c4ccccc4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5ccccc5c4c3)c3c([2H])c([2H])c([2H])c([2H])c3c(-c3cc(-c4ccccc4)cc(-c4ccccc4)c3)c2c1[2H]]

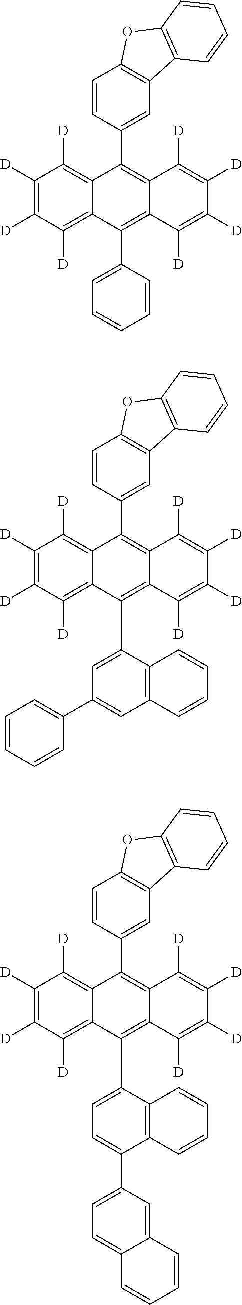 [SMILES: [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3cc(-c4ccccc4)cc4ccccc34)c3c([2H])c([2H])c([2H])c([2H])c3c(-c3ccc4oc5ccccc5c4c3)c2c1[2H] ; [2H]c1c([2H])c([2H])c2c(-c3ccc(-c4ccc5ccccc5c4)c4ccccc34)c3c([2H])c([2H])c([2H])c([2H])c3c(-c3ccc4oc5ccccc5c4c3)c2c1[2H]]

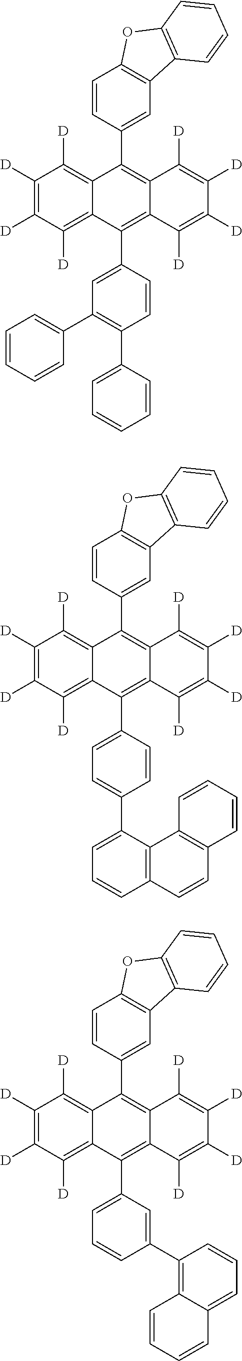

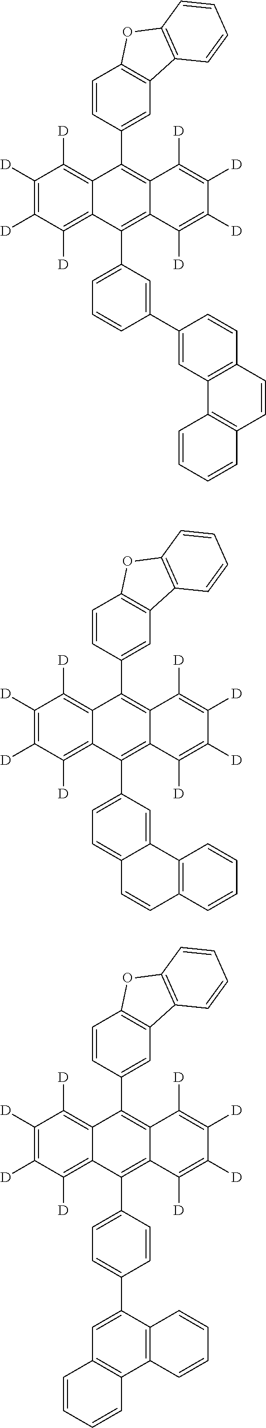

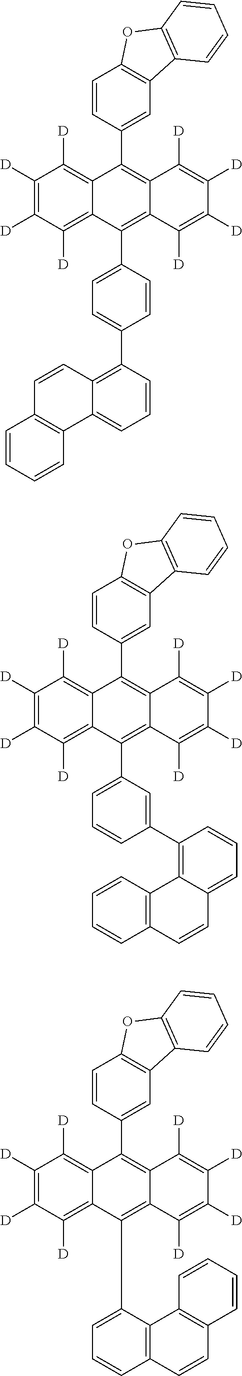 [SMILES: [2H]c1c([2H])c([2H])c2c(-c3ccc4oc5ccccc5c4c3)c3c([2H])c([2H])c([2H])c([2H])c3c(-c3ccc(-c4cccc5c4ccc4ccccc45)cc3)c2c1[2H] ; [2H]c1c([2H])c([2H])c2c(-c3ccc4oc5ccccc5c4c3)c3c([2H])c([2H])c([2H])c([2H])c3c(-c3cccc(-c4cccc5ccc6ccccc6c45)c3)c2c1[2H] ; [2H]c1c([2H])c([2H])c2c(-c3cccc4ccc5ccccc5c34)c3c([2H])c([2H])c([2H])c([2H])c3c(-c3ccc4oc5ccccc5c4c3)c2c1[2H]]

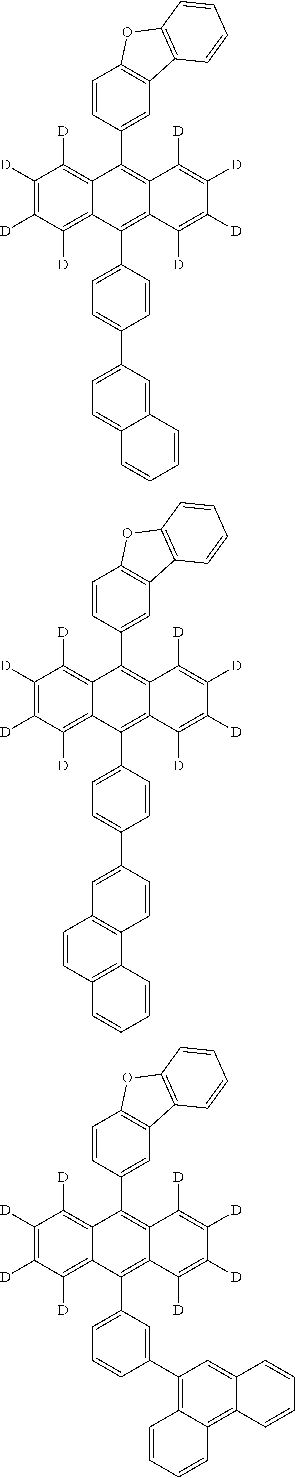

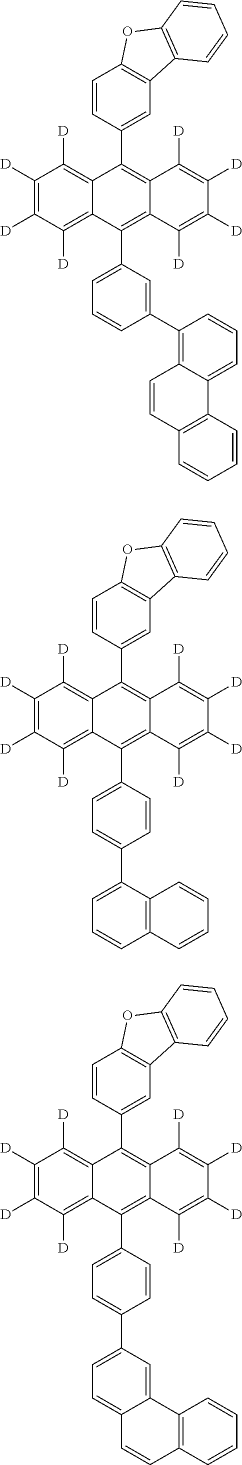

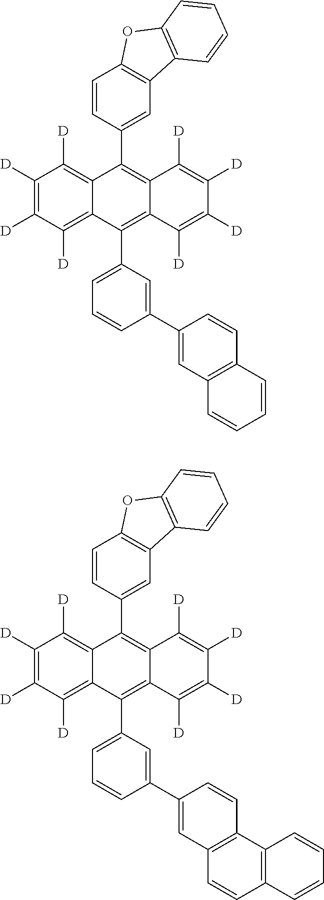 [SMILES: [2H]c1c([2H])c([2H])c2c(-c3ccc4oc5ccccc5c4c3)c3c([2H])c([2H])c([2H])c([2H])c3c(-c3cccc(-c4ccc5ccccc5c4)c3)c2c1[2H] ; [2H]c1c([2H])c([2H])c2c(-c3ccc4oc5ccccc5c4c3)c3c([2H])c([2H])c([2H])c([2H])c3c(-c3cccc(-c4ccc5c(ccc6ccccc65)c4)c3)c2c1[2H]]

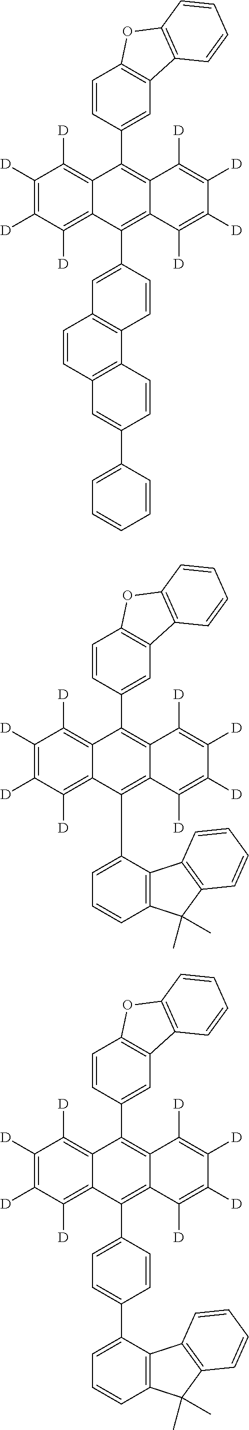

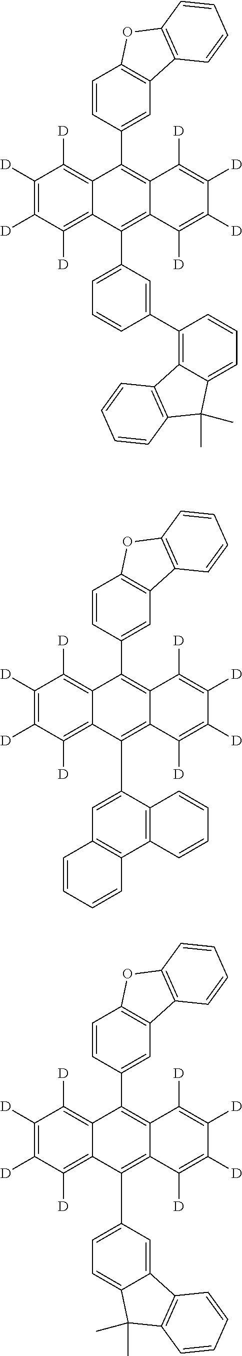 [SMILES: [2H]c1c([2H])c([2H])c2c(-c3ccc4oc5ccccc5c4c3)c3c([2H])c([2H])c([2H])c([2H])c3c(-c3cccc(-c4cccc5c4-c4ccccc4C5(C)C)c3)c2c1[2H] ; [2H]c1c([2H])c([2H])c2c(-c3cc4ccccc4c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4c(c3)-c3ccccc3C4(C)C)c2c1[2H]]

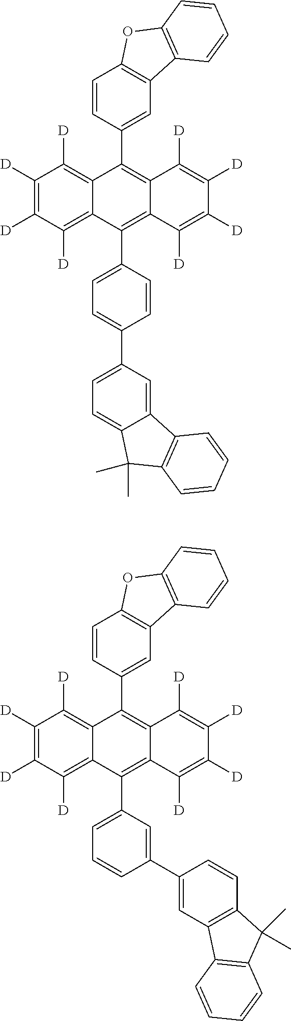 [SMILES: [2H]c1c([2H])c([2H])c2c(-c3ccc4oc5ccccc5c4c3)c3c([2H])c([2H])c([2H])c([2H])c3c(-c3ccc(-c4ccc5c(c4)-c4ccccc4C5(C)C)cc3)c2c1[2H] ; [2H]c1c([2H])c([2H])c2c(-c3ccc4oc5ccccc5c4c3)c3c([2H])c([2H])c([2H])c([2H])c3c(-c3cccc(-c4ccc5c(c4)-c4ccccc4C5(C)C)c3)c2c1[2H]]

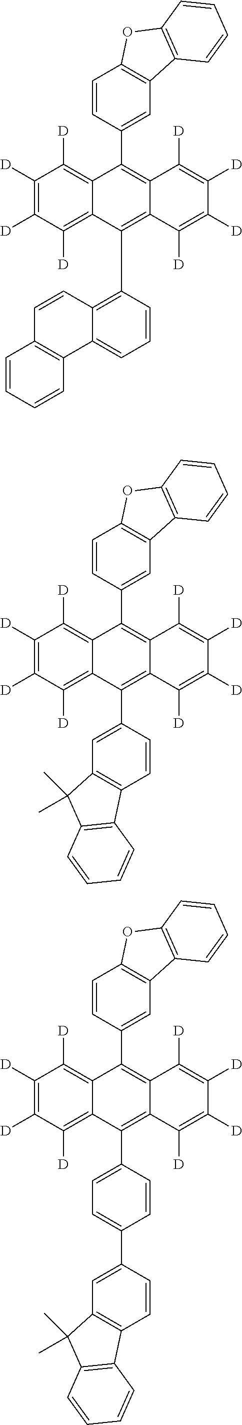 [SMILES: [2H]c1c([2H])c([2H])c2c(-c3cccc4c3ccc3ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4c(c3)C(C)(C)c3ccccc3-4)c2c1[2H] ; [2H]c1c([2H])c([2H])c2c(-c3ccc4oc5ccccc5c4c3)c3c([2H])c([2H])c([2H])c([2H])c3c(-c3ccc(-c4ccc5c(c4)C(C)(C)c4ccccc4-5)cc3)c2c1[2H]]

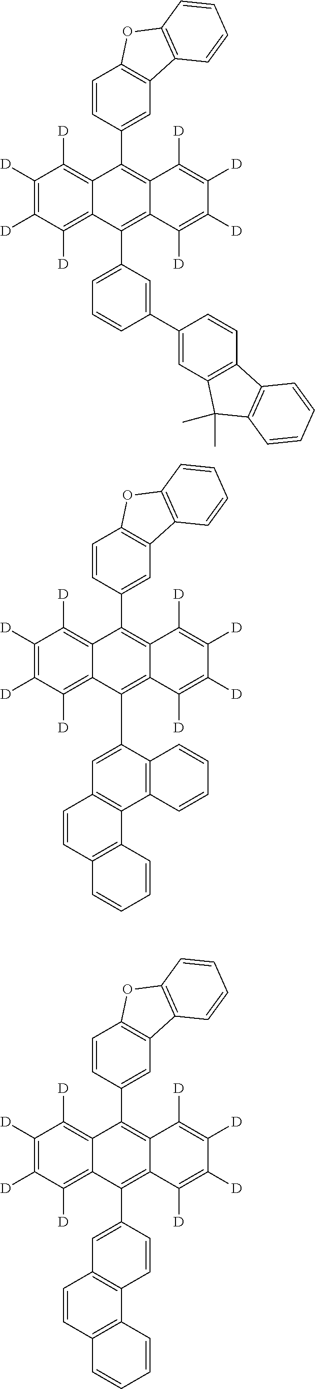

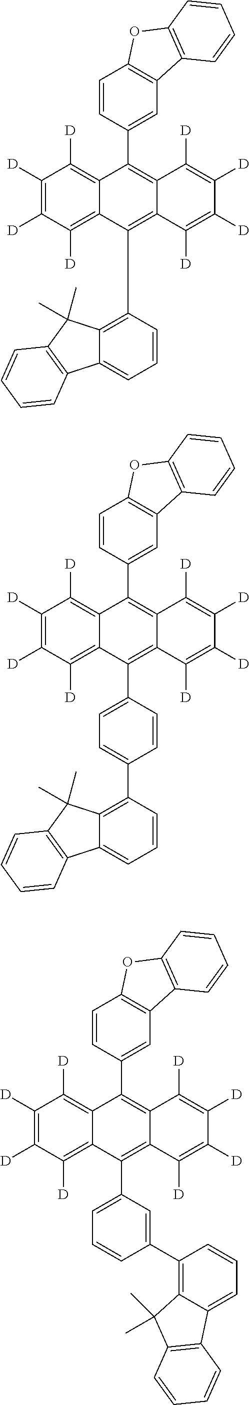 [SMILES: [2H]c1c([2H])c([2H])c2c(-c3cccc4c3C(C)(C)c3ccccc3-4)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4cccc5c4C(C)(C)c4ccccc4-5)cc3)c2c1[2H] ; [2H]c1c([2H])c([2H])c2c(-c3ccc4oc5ccccc5c4c3)c3c([2H])c([2H])c([2H])c([2H])c3c(-c3cccc(-c4cccc5c4C(C)(C)c4ccccc4-5)c3)c2c1[2H]]

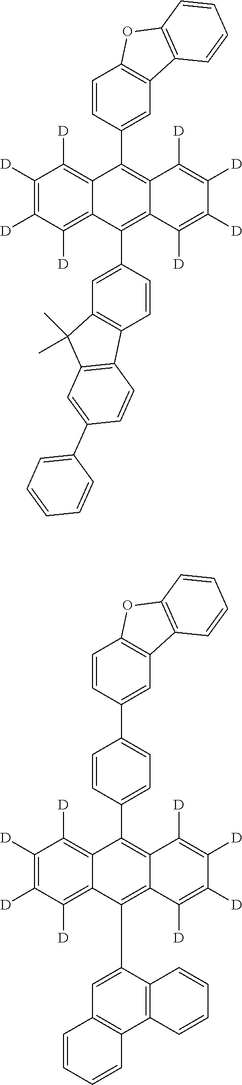 [SMILES: [2H]c1c([2H])c([2H])c2c(-c3ccc4oc5ccccc5c4c3)c3c([2H])c([2H])c([2H])c([2H])c3c(-c3ccc4c(c3)C(C)(C)c3cc(-c5ccccc5)ccc3-4)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5oc6ccccc6c5c4)cc3)c2c1[2H]]

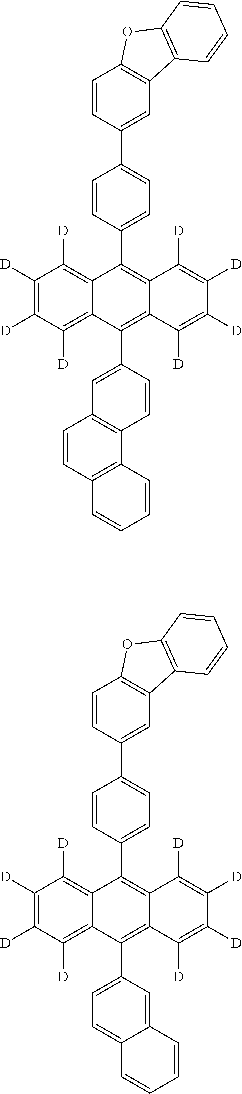 [SMILES: [2H]c1c([2H])c([2H])c2c(-c3ccc4c(ccc5ccccc54)c3)c3c([2H])c([2H])c([2H])c([2H])c3c(-c3ccc(-c4ccc5oc6ccccc6c5c4)cc3)c2c1[2H] ; [2H]c1c([2H])c([2H])c2c(-c3ccc4ccccc4c3)c3c([2H])c([2H])c([2H])c([2H])c3c(-c3ccc(-c4ccc5oc6ccccc6c5c4)cc3)c2c1[2H]]

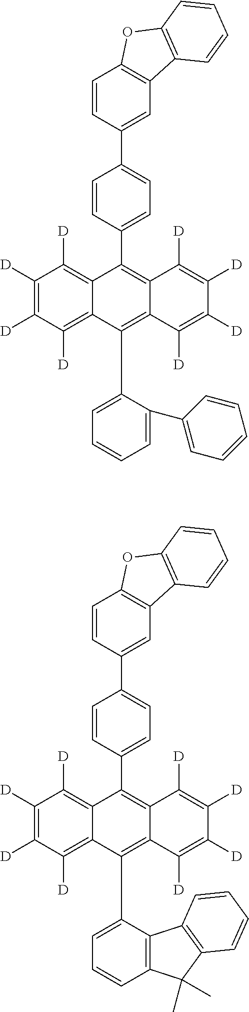 [SMILES: [2H]c1c([2H])c([2H])c2c(-c3ccccc3-c3ccccc3)c3c([2H])c([2H])c([2H])c([2H])c3c(-c3ccc(-c4ccc5oc6ccccc6c5c4)cc3)c2c1[2H] ; [2H]c1c([2H])c([2H])c2c(-c3cccc4c3-c3ccccc3C4(C)C)c3c([2H])c([2H])c([2H])c([2H])c3c(-c3ccc(-c4ccc5oc6ccccc6c5c4)cc3)c2c1[2H]]

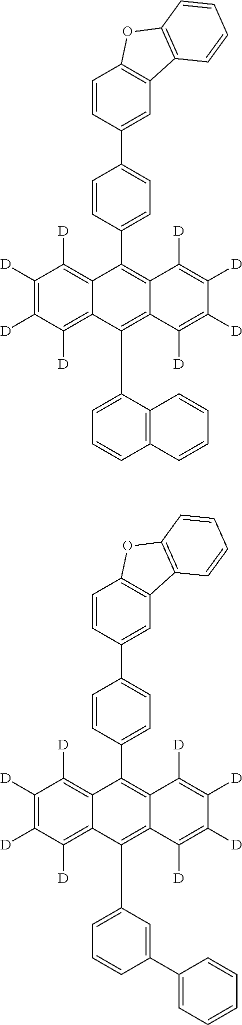 [SMILES: [2H]c1c([2H])c([2H])c2c(-c3cccc4ccccc34)c3c([2H])c([2H])c([2H])c([2H])c3c(-c3ccc(-c4ccc5oc6ccccc6c5c4)cc3)c2c1[2H] ; [2H]c1c([2H])c([2H])c2c(-c3cccc(-c4ccccc4)c3)c3c([2H])c([2H])c([2H])c([2H])c3c(-c3ccc(-c4ccc5oc6ccccc6c5c4)cc3)c2c1[2H]]

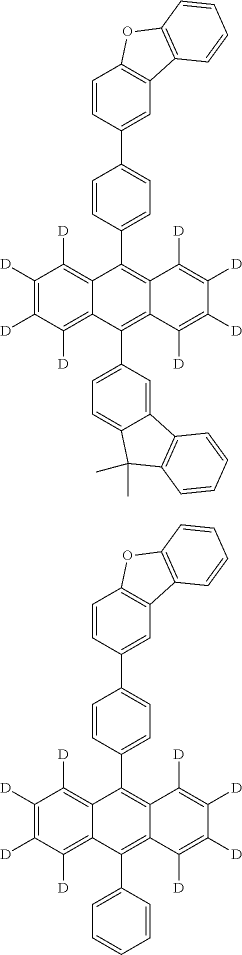

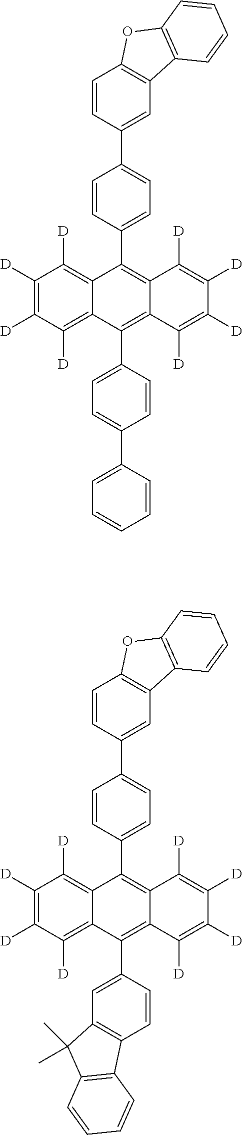 [SMILES: [2H]c1c([2H])c([2H])c2c(-c3ccc(-c4ccc5oc6ccccc6c5c4)cc3)c3c([2H])c([2H])c([2H])c([2H])c3c(-c3ccc(-c4ccccc4)cc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccc5oc6ccccc6c5c4)cc3)c2c1[2H]]

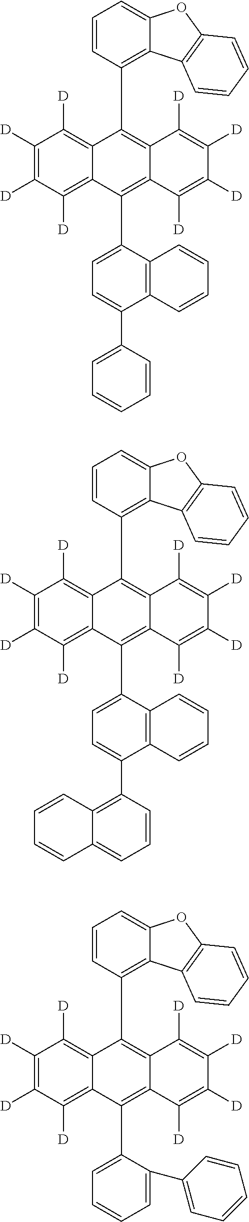

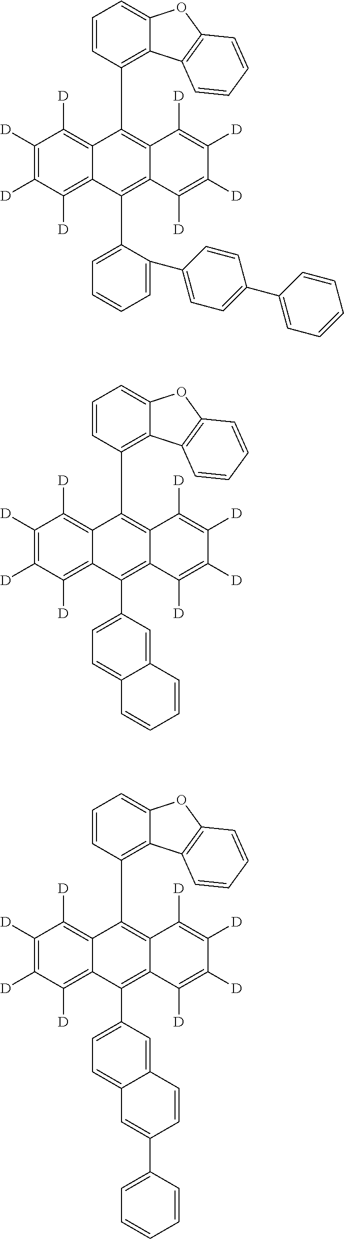 [SMILES: [2H]c1c([2H])c([2H])c2c(-c3cccc4oc5ccccc5c34)c3c([2H])c([2H])c([2H])c([2H])c3c(-c3ccccc3-c3ccc(-c4ccccc4)cc3)c2c1[2H] ; [2H]c1c([2H])c([2H])c2c(-c3cccc4oc5ccccc5c34)c3c([2H])c([2H])c([2H])c([2H])c3c(-c3ccc4ccccc4c3)c2c1[2H] ; [2H]c1c([2H])c([2H])c2c(-c3cccc4oc5ccccc5c34)c3c([2H])c([2H])c([2H])c([2H])c3c(-c3ccc4cc(-c5ccccc5)ccc4c3)c2c1[2H]]

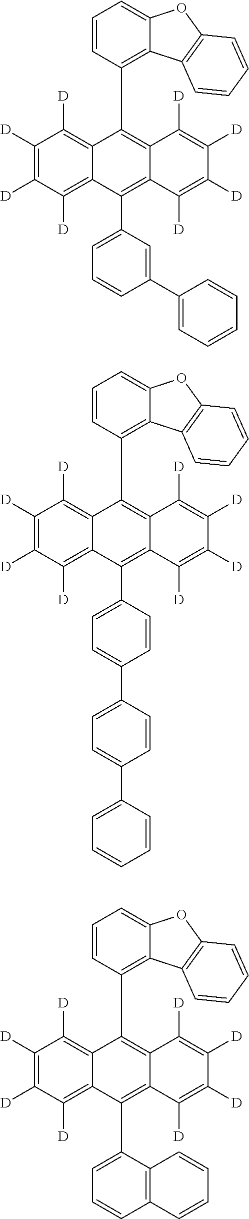

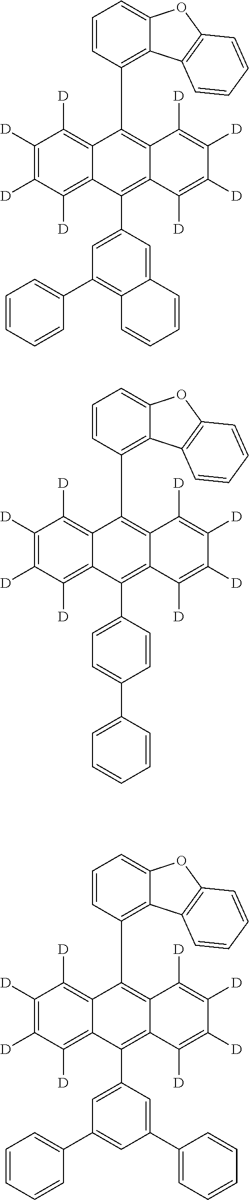 [SMILES: [2H]c1c([2H])c([2H])c2c(-c3cccc4oc5ccccc5c34)c3c([2H])c([2H])c([2H])c([2H])c3c(-c3cc(-c4ccccc4)c4ccccc4c3)c2c1[2H] ; [2H]c1c([2H])c([2H])c2c(-c3cccc4oc5ccccc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5ccccc5c34)c3c([2H])c([2H])c([2H])c([2H])c3c(-c3cc(-c4ccccc4)cc(-c4ccccc4)c3)c2c1[2H]]

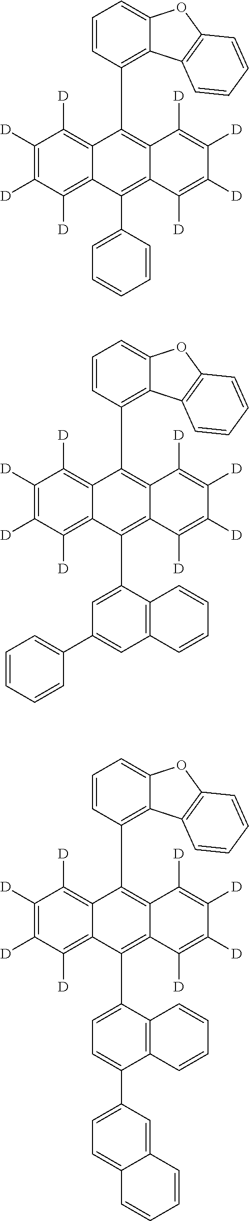

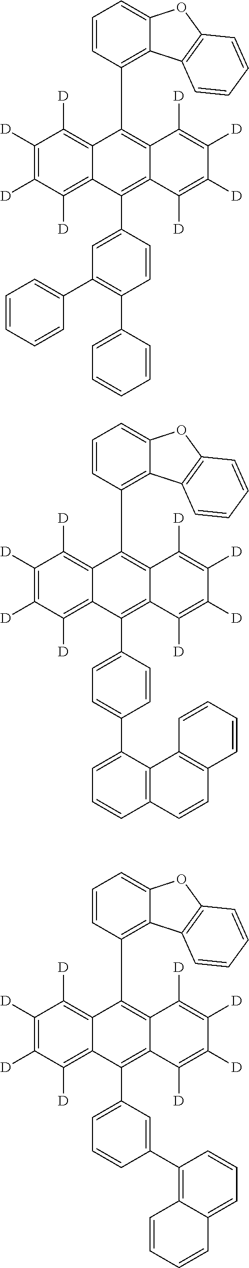

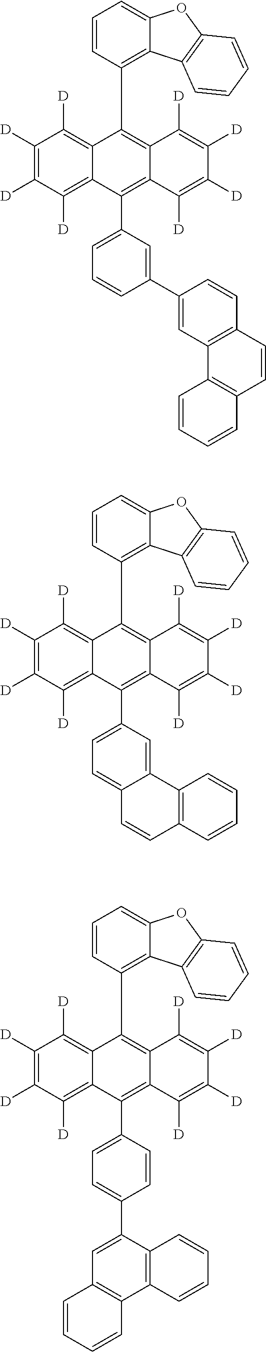 [SMILES: [2H]c1c([2H])c([2H])c2c(-c3cccc4oc5ccccc5c34)c3c([2H])c([2H])c([2H])c([2H])c3c(-c3cccc(-c4ccc5ccc6ccccc6c5c4)c3)c2c1[2H] ; [2H]c1c([2H])c([2H])c2c(-c3cccc4oc5ccccc5c34)c3c([2H])c([2H])c([2H])c([2H])c3c(-c3ccc4ccc5ccccc5c4c3)c2c1[2H] ; [2H]c1c([2H])c([2H])c2c(-c3cccc4oc5ccccc5c34)c3c([2H])c([2H])c([2H])c([2H])c3c(-c3ccc(-c4cc5ccccc5c5ccccc45)cc3)c2c1[2H]]

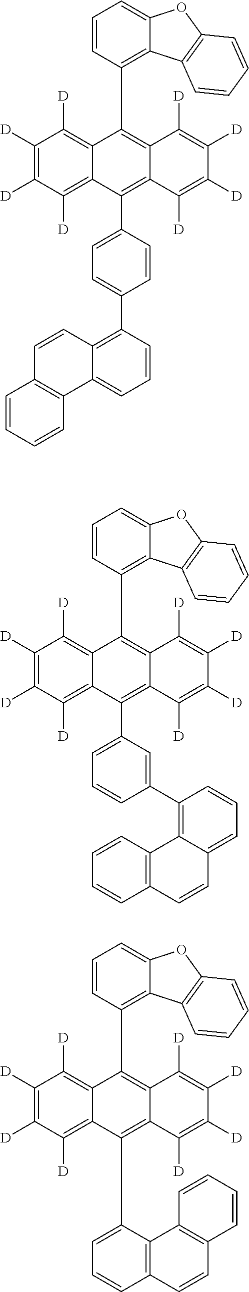

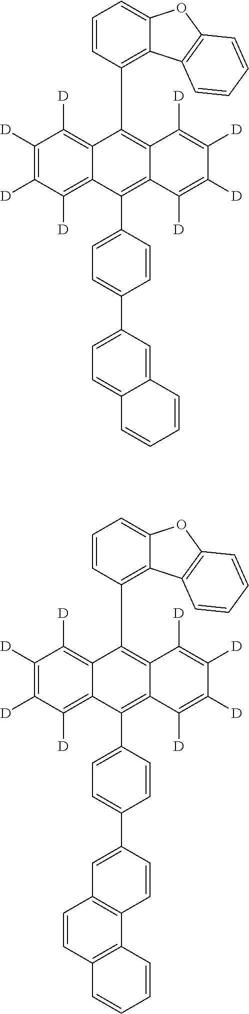 [SMILES: [2H]c1c([2H])c([2H])c2c(-c3cccc4oc5ccccc5c34)c3c([2H])c([2H])c([2H])c([2H])c3c(-c3ccc(-c4ccc5ccccc5c4)cc3)c2c1[2H] ; [2H]c1c([2H])c([2H])c2c(-c3cccc4oc5ccccc5c34)c3c([2H])c([2H])c([2H])c([2H])c3c(-c3ccc(-c4ccc5c(ccc6ccccc65)c4)cc3)c2c1[2H]]

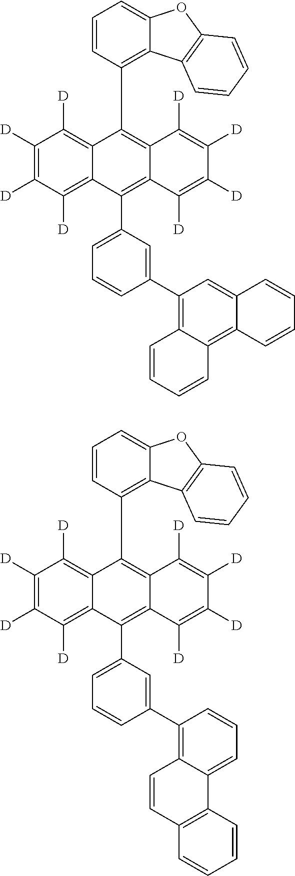

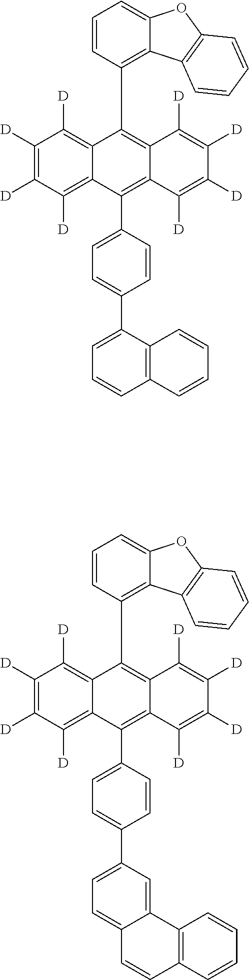 [SMILES: [2H]c1c([2H])c([2H])c2c(-c3cccc4oc5ccccc5c34)c3c([2H])c([2H])c([2H])c([2H])c3c(-c3ccc(-c4cccc5ccccc45)cc3)c2c1[2H] ; [2H]c1c([2H])c([2H])c2c(-c3cccc4oc5ccccc5c34)c3c([2H])c([2H])c([2H])c([2H])c3c(-c3ccc(-c4ccc5ccc6ccccc6c5c4)cc3)c2c1[2H]]

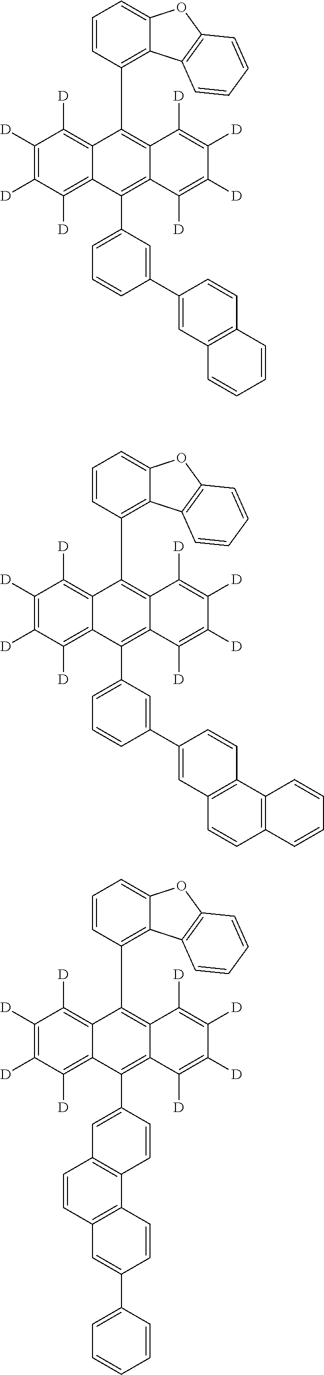 [SMILES: [2H]c1c([2H])c([2H])c2c(-c3cccc4oc5ccccc5c34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4oc5ccccc5c34)c3c([2H])c([2H])c([2H])c([2H])c3c(-c3cccc(-c4ccc5c(ccc6ccccc65)c4)c3)c2c1[2H] ; [2H]c1c([2H])c([2H])c2c(-c3cccc4oc5ccccc5c34)c3c([2H])c([2H])c([2H])c([2H])c3c(-c3ccc4c(ccc5cc(-c6ccccc6)ccc54)c3)c2c1[2H]]

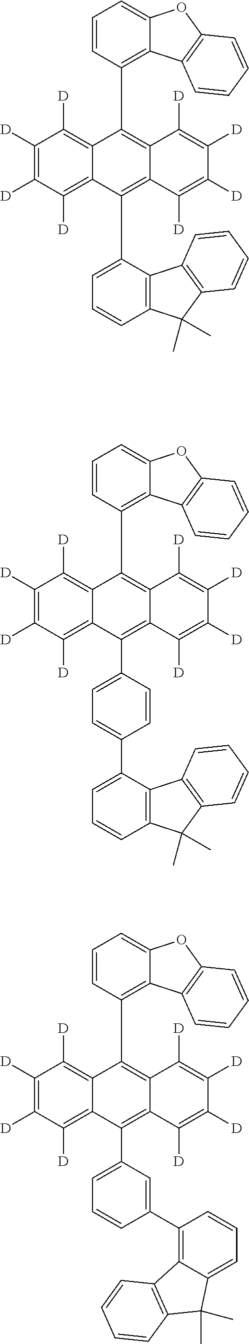 [SMILES: [2H]c1c([2H])c([2H])c2c(-c3cccc4oc5ccccc5c34)c3c([2H])c([2H])c([2H])c([2H])c3c(-c3cccc4c3-c3ccccc3C4(C)C)c2c1[2H] ; [2H]c1c([2H])c([2H])c2c(-c3cccc4oc5ccccc5c34)c3c([2H])c([2H])c([2H])c([2H])c3c(-c3ccc(-c4cccc5c4-c4ccccc4C5(C)C)cc3)c2c1[2H] ; [2H]c1c([2H])c([2H])c2c(-c3cccc4oc5ccccc5c34)c3c([2H])c([2H])c([2H])c([2H])c3c(-c3cccc(-c4cccc5c4-c4ccccc4C5(C)C)c3)c2c1[2H]]

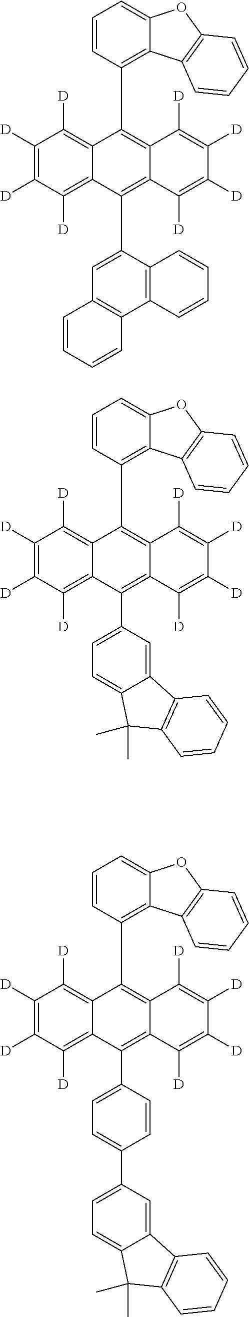

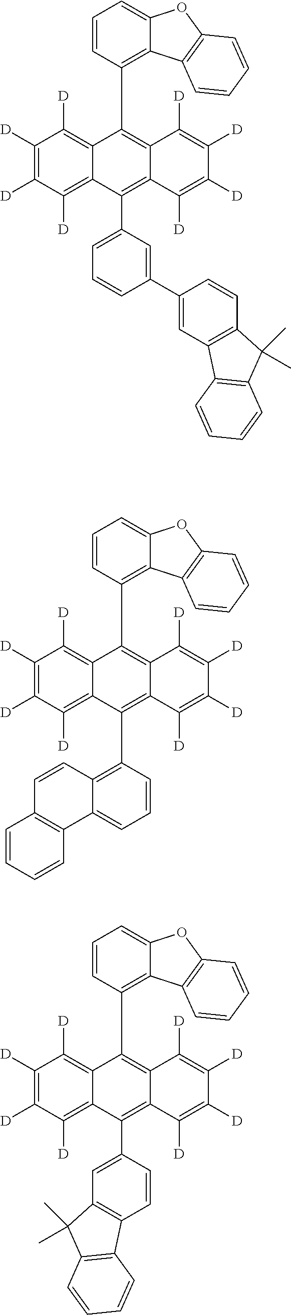 [SMILES: [2H]c1c([2H])c([2H])c2c(-c3cccc4oc5ccccc5c34)c3c([2H])c([2H])c([2H])c([2H])c3c(-c3cccc(-c4ccc5c(c4)-c4ccccc4C5(C)C)c3)c2c1[2H] ; [2H]c1c([2H])c([2H])c2c(-c3cccc4oc5ccccc5c34)c3c([2H])c([2H])c([2H])c([2H])c3c(-c3cccc4c3ccc3ccccc34)c2c1[2H] ; [2H]c1c([2H])c([2H])c2c(-c3cccc4oc5ccccc5c34)c3c([2H])c([2H])c([2H])c([2H])c3c(-c3ccc4c(c3)C(C)(C)c3ccccc3-4)c2c1[2H]]

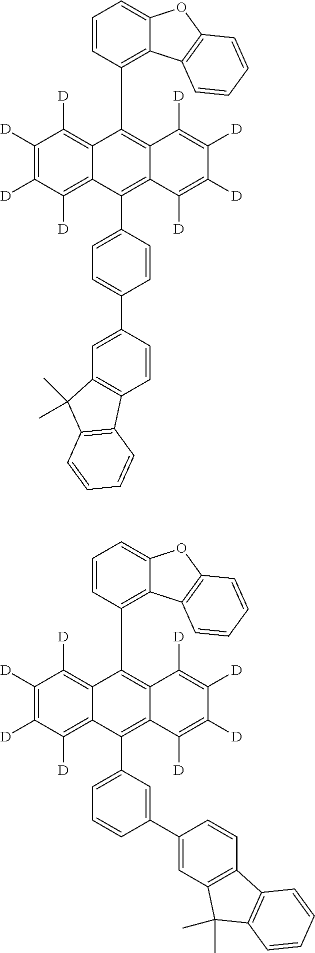 [SMILES: [2H]c1c([2H])c([2H])c2c(-c3cccc4oc5ccccc5c34)c3c([2H])c([2H])c([2H])c([2H])c3c(-c3ccc(-c4ccc5c(c4)C(C)(C)c4ccccc4-5)cc3)c2c1[2H] ; [2H]c1c([2H])c([2H])c2c(-c3cccc4oc5ccccc5c34)c3c([2H])c([2H])c([2H])c([2H])c3c(-c3cccc(-c4ccc5c(c4)C(C)(C)c4ccccc4-5)c3)c2c1[2H]]

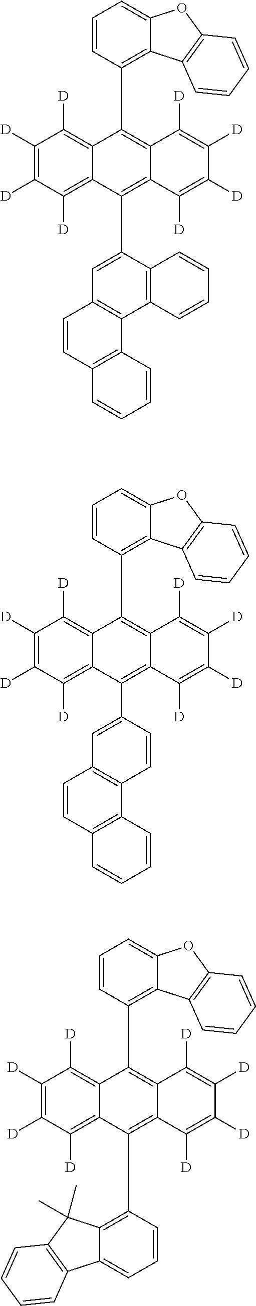 [SMILES: [2H]c1c([2H])c([2H])c2c(-c3cccc4oc5ccccc5c34)c3c([2H])c([2H])c([2H])c([2H])c3c(-c3cc4ccc5ccccc5c4c4ccccc34)c2c1[2H] ; [2H]c1c([2H])c([2H])c2c(-c3cccc4oc5ccccc5c34)c3c([2H])c([2H])c([2H])c([2H])c3c(-c3ccc4c(ccc5ccccc54)c3)c2c1[2H] ; [2H]c1c([2H])c([2H])c2c(-c3cccc4oc5ccccc5c34)c3c([2H])c([2H])c([2H])c([2H])c3c(-c3cccc4c3C(C)(C)c3ccccc3-4)c2c1[2H]]

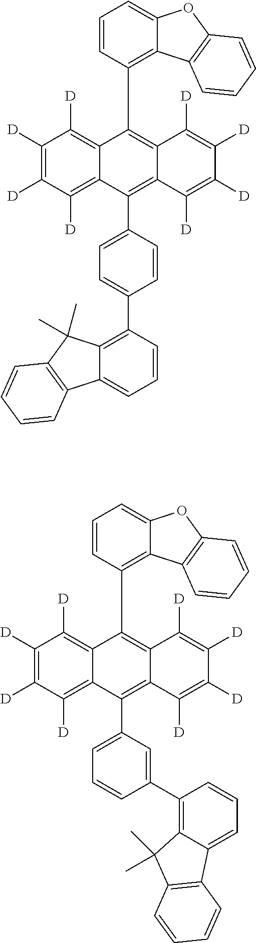 [SMILES: [2H]c1c([2H])c([2H])c2c(-c3cccc4oc5ccccc5c34)c3c([2H])c([2H])c([2H])c([2H])c3c(-c3ccc(-c4cccc5c4C(C)(C)c4ccccc4-5)cc3)c2c1[2H] ; [2H]c1c([2H])c([2H])c2c(-c3cccc4oc5ccccc5c34)c3c([2H])c([2H])c([2H])c([2H])c3c(-c3cccc(-c4cccc5c4C(C)(C)c4ccccc4-5)c3)c2c1[2H]]

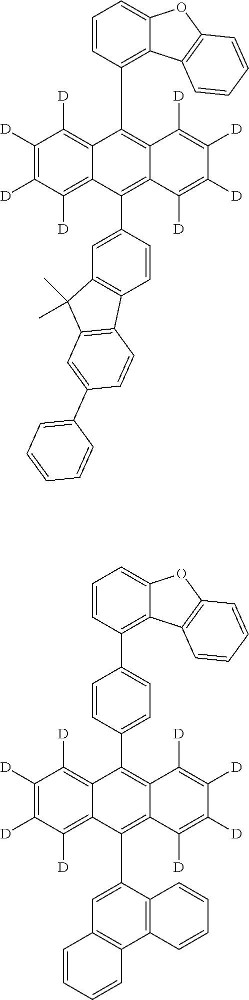 [SMILES: [2H]c1c([2H])c([2H])c2c(-c3cccc4oc5ccccc5c34)c3c([2H])c([2H])c([2H])c([2H])c3c(-c3ccc4c(c3)C(C)(C)c3cc(-c5ccccc5)ccc3-4)c2c1[2H] ; [2H]c1c([2H])c([2H])c2c(-c3cc4ccccc4c4ccccc34)c3c([2H])c([2H])c([2H])c([2H])c3c(-c3ccc(-c4cccc5oc6ccccc6c45)cc3)c2c1[2H]]

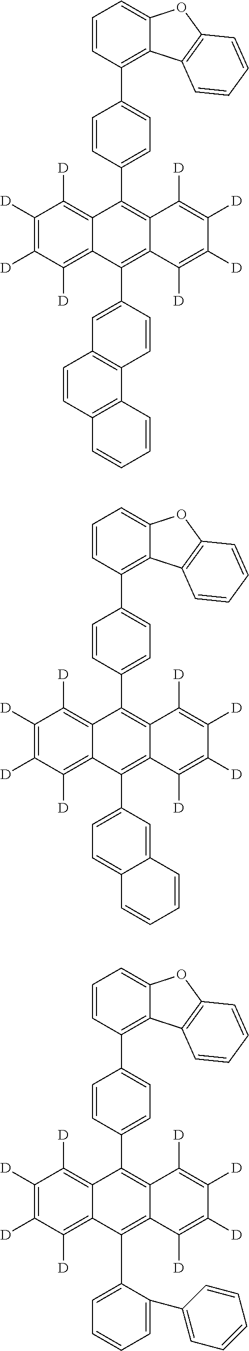

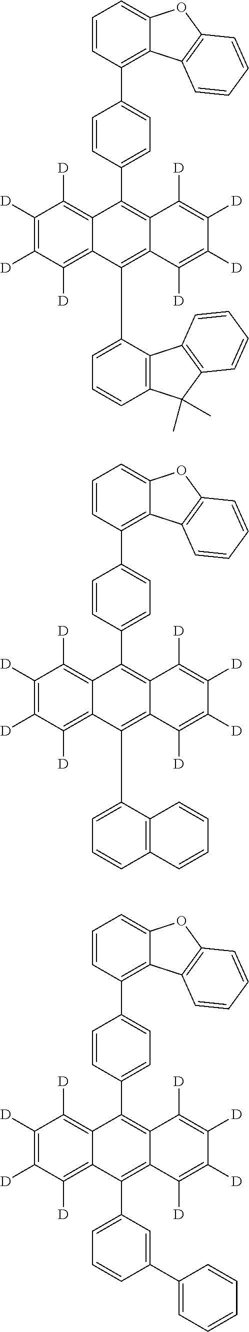

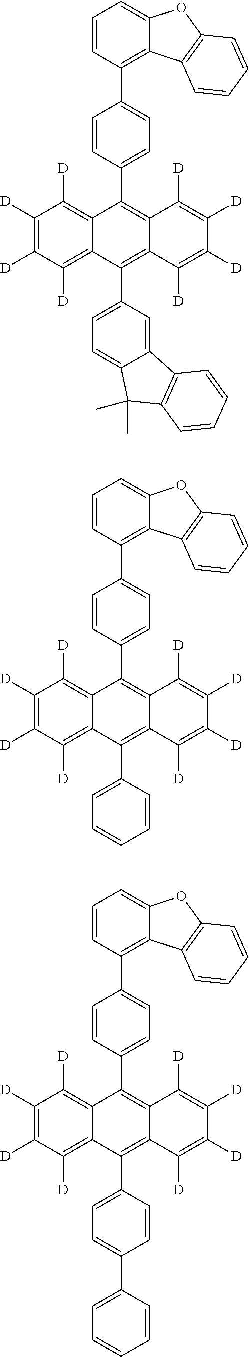

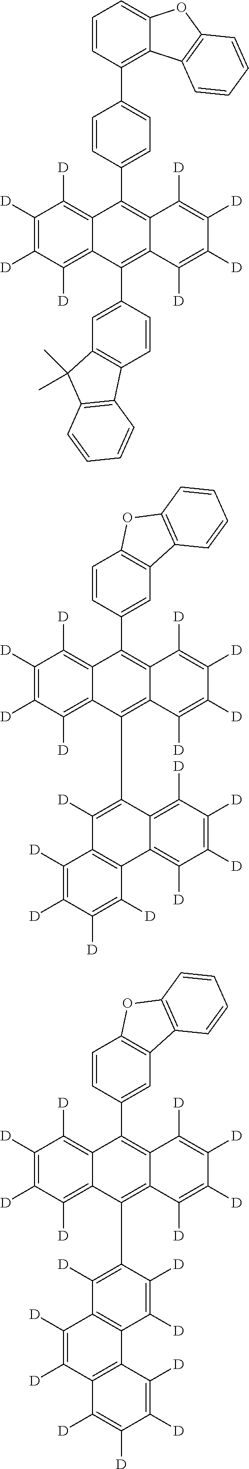 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4cccc5oc6ccccc6c45)cc3)c2c1[2H] ; [2H]c1c([2H])c([2H])c2c(-c3c([2H])c4c([2H])c([2H])c([2H])c([2H])c4c4c([2H])c([2H])c([2H])c([2H])c34)c3c([2H])c([2H])c([2H])c([2H])c3c(-c3ccc4oc5ccccc5c4c3)c2c1[2H] ; [2H]c1c([2H])c([2H])c2c(-c3c([2H])c([2H])c4c(c3[2H])c([2H])c([2H])c3c([2H])c([2H])c([2H])c([2H])c34)c3c([2H])c([2H])c([2H])c([2H])c3c(-c3ccc4oc5ccccc5c4c3)c2c1[2H]]

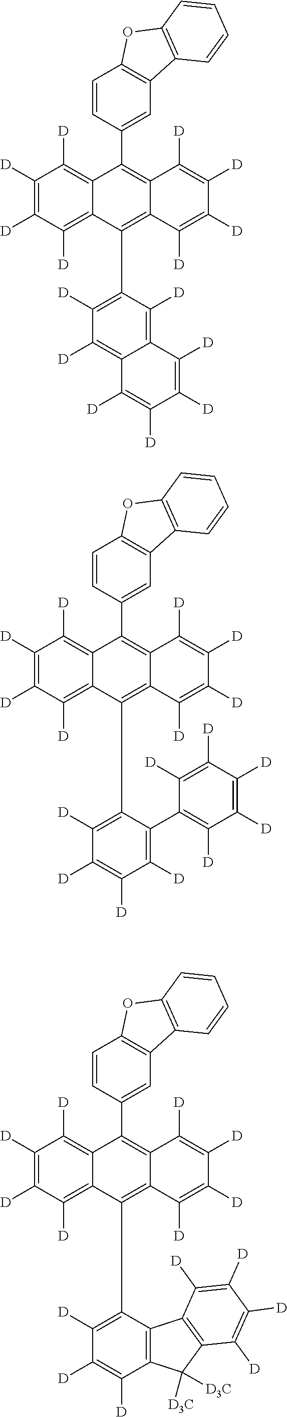

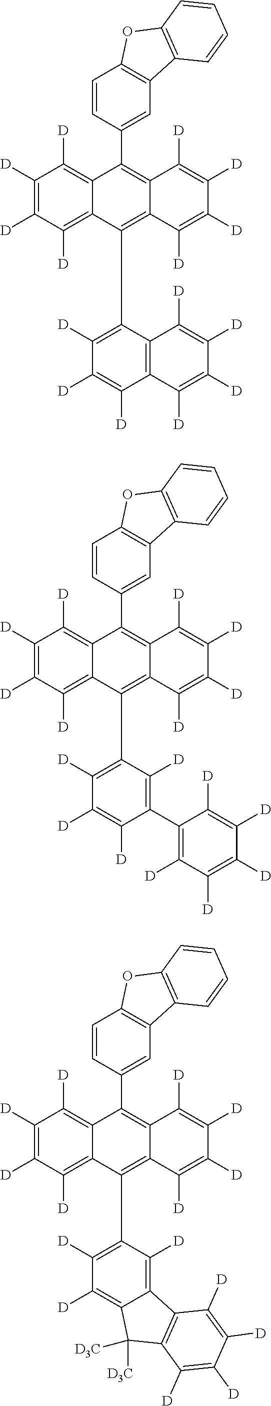

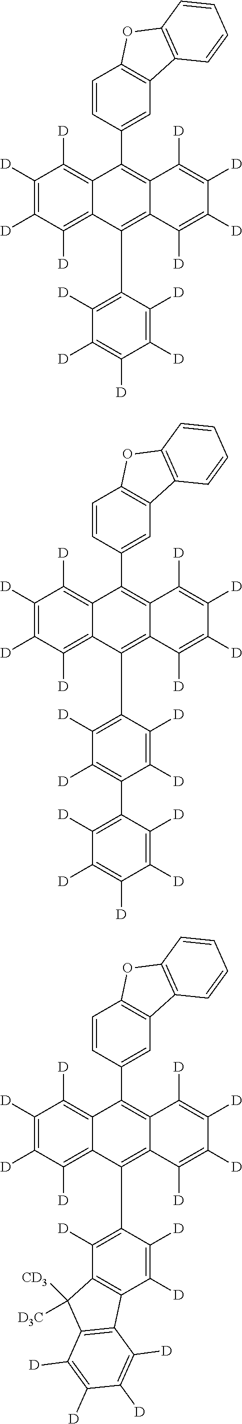 [SMILES: [2H]c1c([2H])c([2H])c(-c2c3c([2H])c([2H])c([2H])c([2H])c3c(-c3ccc4oc5ccccc5c4c3)c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4ccc5oc6ccccc6c5c4)c4c([2H])c([2H])c([2H])c([2H])c34)c([2H])c2[2H])c([2H])c1[2H] ; [2H]c1c([2H])c([2H])c2c(c1[2H])-c1c([2H])c([2H])c(-c3c4c([2H])c([2H])c([2H])c([2H])c4c(-c4ccc5oc6ccccc6c5c4)c4c([2H])c([2H])c([2H])c([2H])c34)c([2H])c1C2(C([2H])([2H])[2H])C([2H])([2H])[2H]]

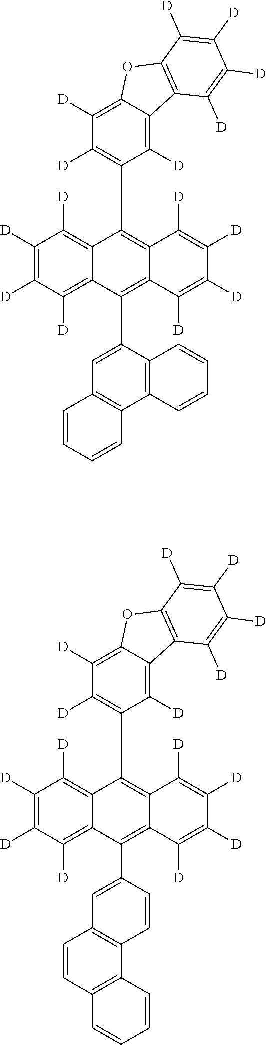 [SMILES: [2H]c1c([2H])c([2H])c2c(oc3c([2H])c([2H])c(-c4c5c([2H])c([2H])c([2H])c([2H])c5c(-c5cc6ccccc6c6ccccc56)c5c([2H])c([2H])c([2H])c([2H])c45)c([2H])c32)c1[2H] ; [2H]c1c([2H])c([2H])c2c(oc3c([2H])c([2H])c(-c4c5c([2H])c([2H])c([2H])c([2H])c5c(-c5ccc6c(ccc7ccccc76)c5)c5c([2H])c([2H])c([2H])c([2H])c45)c([2H])c32)c1[2H]]

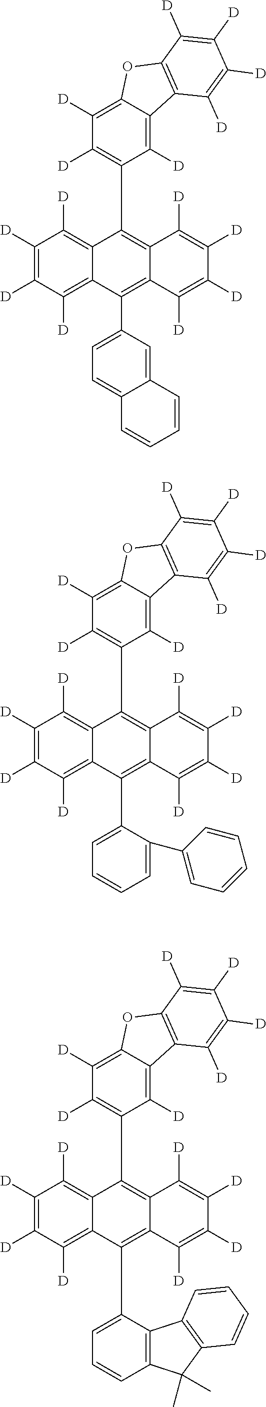

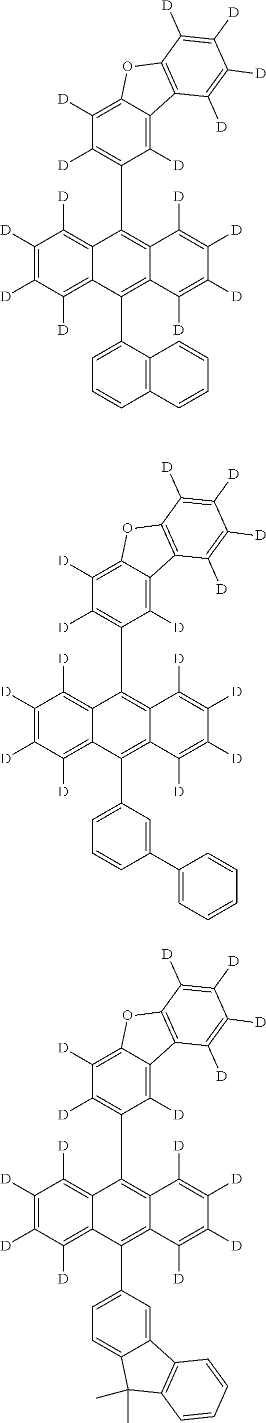 [SMILES: [2H]c1c([2H])c([2H])c2c(oc3c([2H])c([2H])c(-c4c5c([2H])c([2H])c([2H])c([2H])c5c(-c5cccc6ccccc56)c5c([2H])c([2H])c([2H])c([2H])c45)c([2H])c32)c1[2H] ; [2H]c1c([2H])c([2H])c2c(oc3c([2H])c([2H])c(-c4c5c([2H])c([2H])c([2H])c([2H])c5c(-c5cccc(-c6ccccc6)c5)c5c([2H])c([2H])c([2H])c([2H])c45)c([2H])c32)c1[2H] ; [2H]c1c([2H])c([2H])c2c(oc3c([2H])c([2H])c(-c4c5c([2H])c([2H])c([2H])c([2H])c5c(-c5ccc6c(c5)-c5ccccc5C6(C)C)c5c([2H])c([2H])c([2H])c([2H])c45)c([2H])c32)c1[2H]]

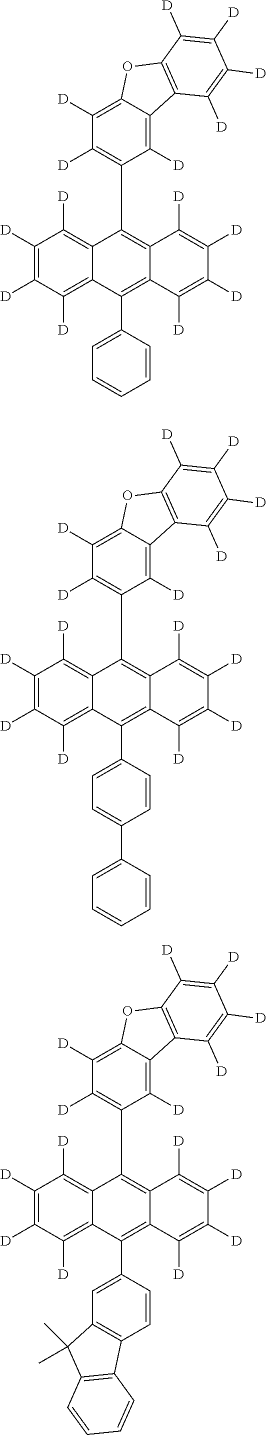

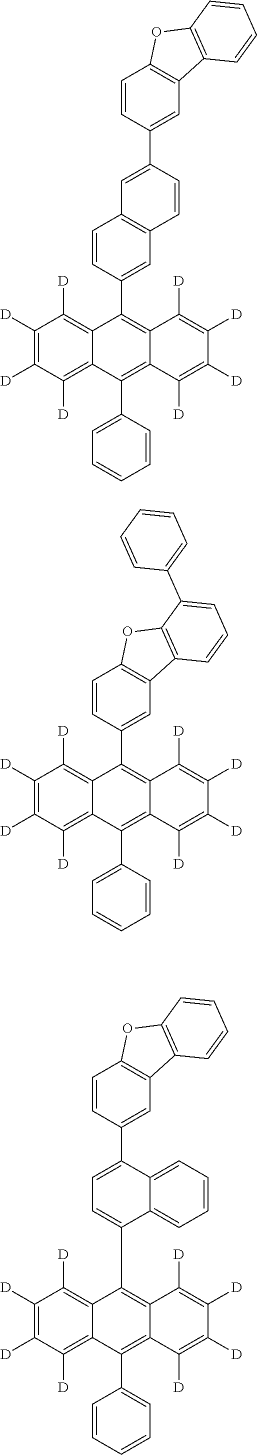

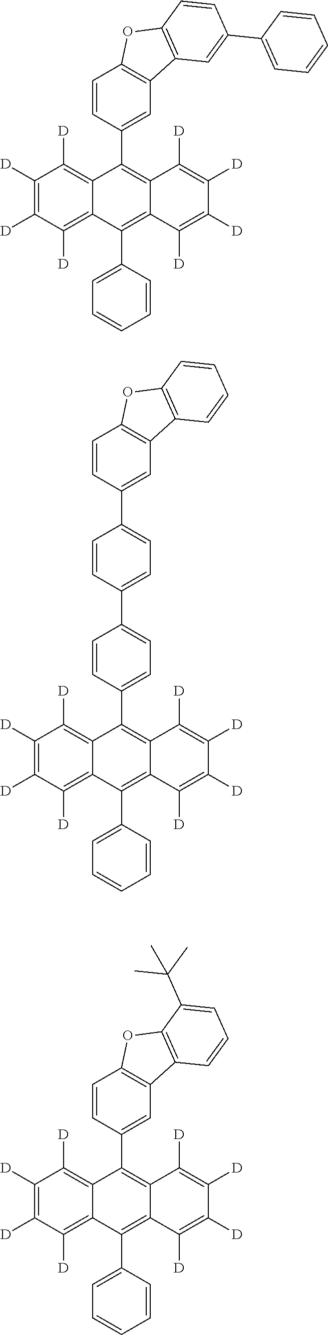 [SMILES: [2H]c1c([2H])c([2H])c2c(-c3ccc4oc5ccc(-c6ccccc6)cc5c4c3)c3c([2H])c([2H])c([2H])c([2H])c3c(-c3ccccc3)c2c1[2H] ; [2H]c1c([2H])c([2H])c2c(-c3ccc(-c4ccc(-c5ccc6oc7ccccc7c6c5)cc4)cc3)c3c([2H])c([2H])c([2H])c([2H])c3c(-c3ccccc3)c2c1[2H] ; [2H]c1c([2H])c([2H])c2c(-c3ccc4oc5c(C(C)(C)C)cccc5c4c3)c3c([2H])c([2H])c([2H])c([2H])c3c(-c3ccccc3)c2c1[2H]]

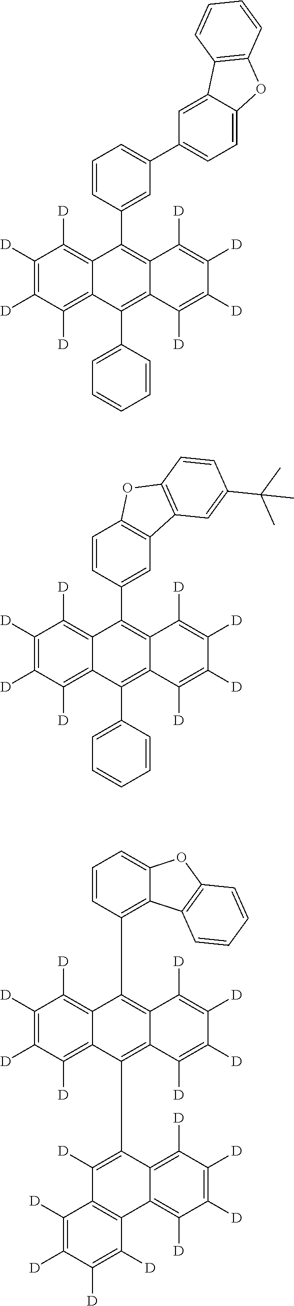 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3ccc4oc5ccc(C(C)(C)C)cc5c4c3)c3c([2H])c([2H])c([2H])c([2H])c3c(-c3ccccc3)c2c1[2H] ; [2H]c1c([2H])c([2H])c2c(-c3cccc4oc5ccccc5c34)c3c([2H])c([2H])c([2H])c([2H])c3c(-c3c([2H])c4c([2H])c([2H])c([2H])c([2H])c4c4c([2H])c([2H])c([2H])c([2H])c34)c2c1[2H]]

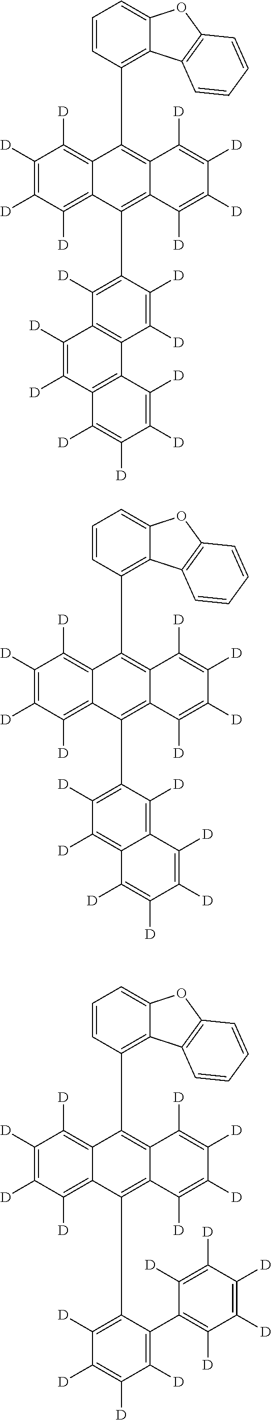

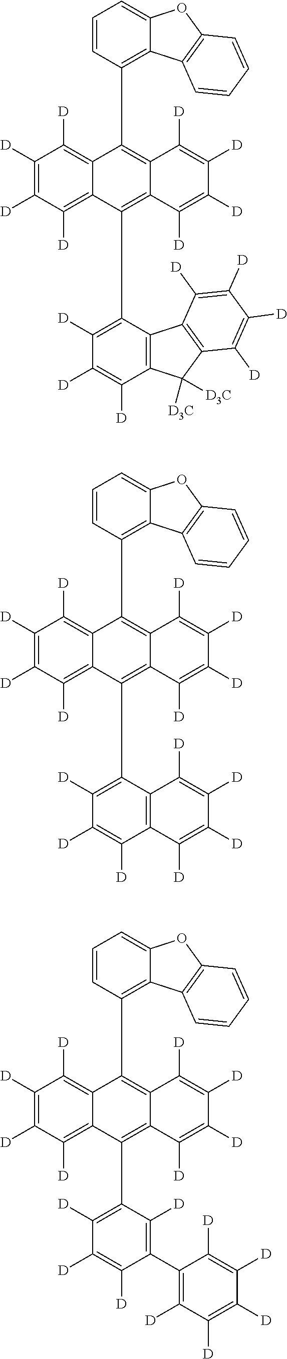 [SMILES: [2H]c1c([2H])c([2H])c2c(c1[2H])-c1c(-c3c4c([2H])c([2H])c([2H])c([2H])c4c(-c4cccc5oc6ccccc6c45)c4c([2H])c([2H])c([2H])c([2H])c34)c([2H])c([2H])c([2H])c1C2(C([2H])([2H])[2H])C([2H])([2H])[2H] ; [2H]c1c([2H])c([2H])c2c(-c3c4c([2H])c([2H])c([2H])c([2H])c4c(-c4cccc5oc6ccccc6c45)c4c([2H])c([2H])c([2H])c([2H])c34)c([2H])c([2H])c([2H])c2c1[2H] ; [2H]c1c([2H])c([2H])c(-c2c([2H])c([2H])c([2H])c(-c3c4c([2H])c([2H])c([2H])c([2H])c4c(-c4cccc5oc6ccccc6c45)c4c([2H])c([2H])c([2H])c([2H])c34)c2[2H])c([2H])c1[2H]]

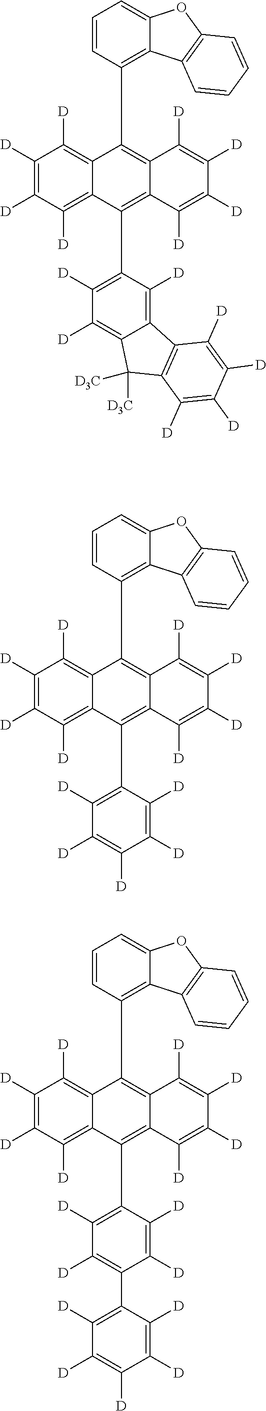 [SMILES: [2H]c1c([2H])c([2H])c2c(c1[2H])-c1c([2H])c(-c3c4c([2H])c([2H])c([2H])c([2H])c4c(-c4cccc5oc6ccccc6c45)c4c([2H])c([2H])c([2H])c([2H])c34)c([2H])c([2H])c1C2(C([2H])([2H])[2H])C([2H])([2H])[2H] ; [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4cccc5oc6ccccc6c45)c4c([2H])c([2H])c([2H])c([2H])c34)c([2H])c2[2H])c([2H])c1[2H]]

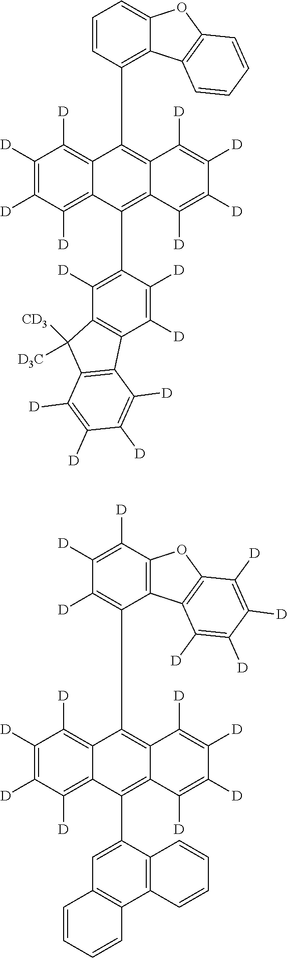 [SMILES: [2H]c1c([2H])c([2H])c2c(c1[2H])-c1c([2H])c([2H])c(-c3c4c([2H])c([2H])c([2H])c([2H])c4c(-c4cccc5oc6ccccc6c45)c4c([2H])c([2H])c([2H])c([2H])c34)c([2H])c1C2(C([2H])([2H])[2H])C([2H])([2H])[2H] ; [2H]c1c([2H])c([2H])c2c(oc3c([2H])c([2H])c([2H])c(-c4c5c([2H])c([2H])c([2H])c([2H])c5c(-c5cc6ccccc6c6ccccc56)c5c([2H])c([2H])c([2H])c([2H])c45)c32)c1[2H]]

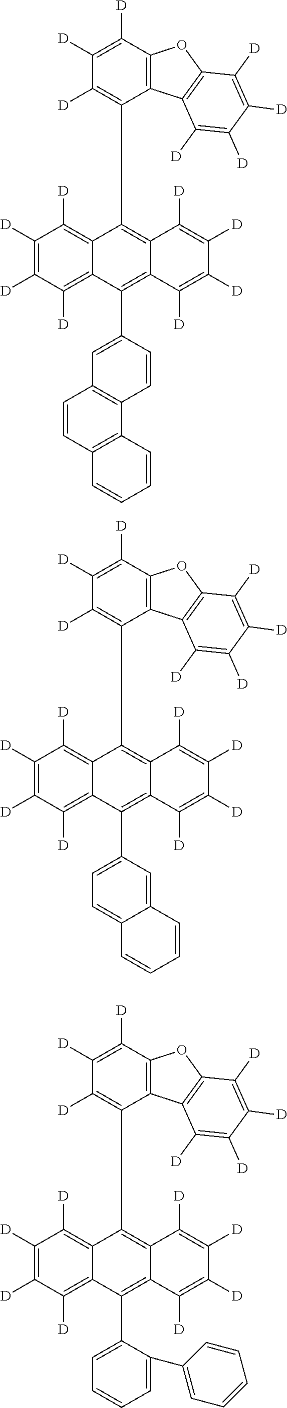

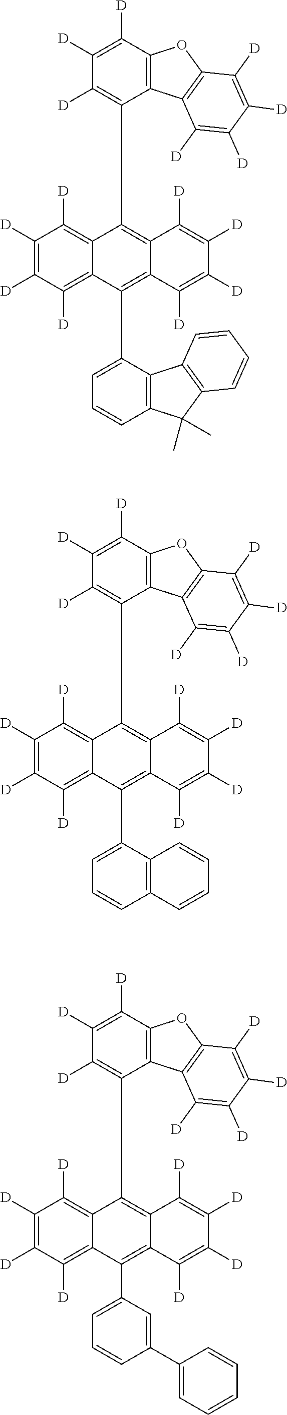 [SMILES: [2H]c1c([2H])c([2H])c2c(oc3c([2H])c([2H])c([2H])c(-c4c5c([2H])c([2H])c([2H])c([2H])c5c(-c5cccc6c5-c5ccccc5C6(C)C)c5c([2H])c([2H])c([2H])c([2H])c45)c32)c1[2H] ; [2H]c1c([2H])c([2H])c2c(oc3c([2H])c([2H])c([2H])c(-c4c5c([2H])c([2H])c([2H])c([2H])c5c(-c5cccc6ccccc56)c5c([2H])c([2H])c([2H])c([2H])c45)c32)c1[2H] ; [2H]c1c([2H])c([2H])c2c(oc3c([2H])c([2H])c([2H])c(-c4c5c([2H])c([2H])c([2H])c([2H])c5c(-c5cccc(-c6ccccc6)c5)c5c([2H])c([2H])c([2H])c([2H])c45)c32)c1[2H]]

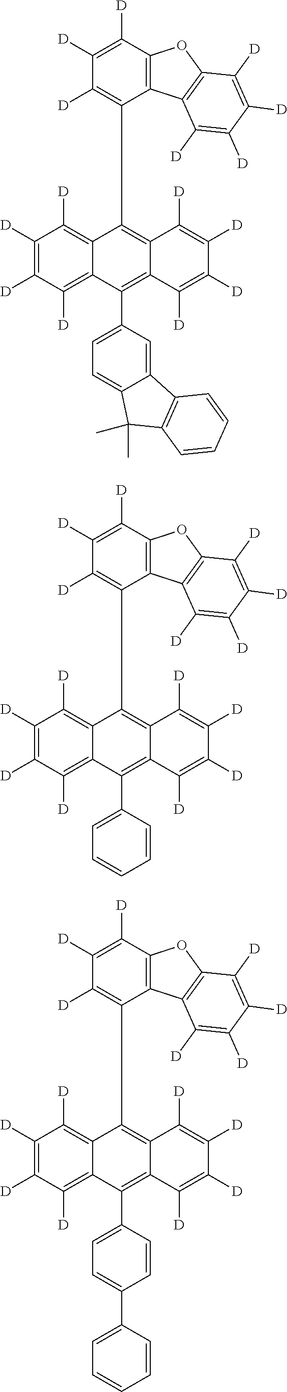

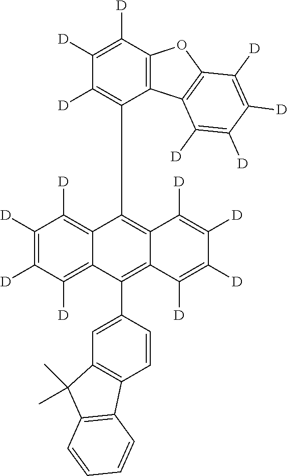 [SMILES: [2H]c1c([2H])c([2H])c2c(oc3c([2H])c([2H])c([2H])c(-c4c5c([2H])c([2H])c([2H])c([2H])c5c(-c5ccc6c(c5)C(C)(C)c5ccccc5-6)c5c([2H])c([2H])c([2H])c([2H])c45)c32)c1[2H]]

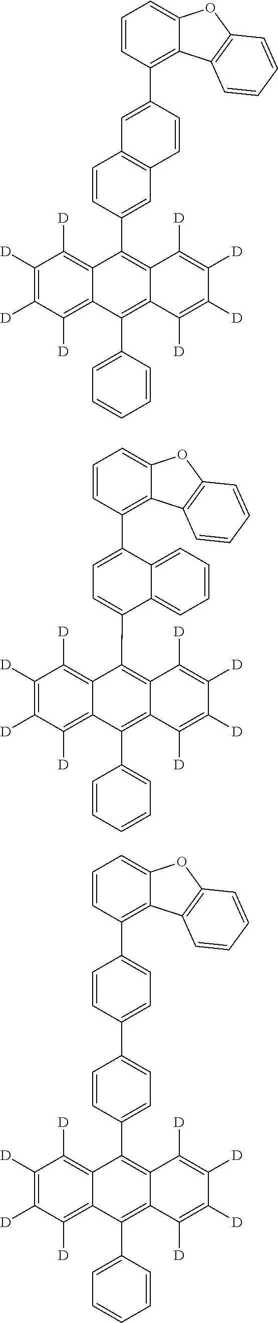

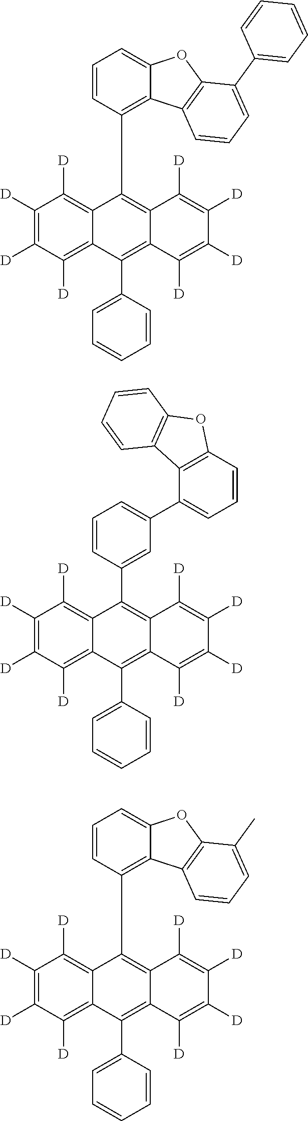

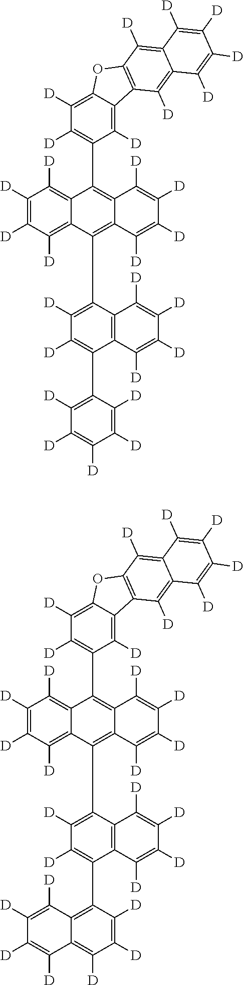

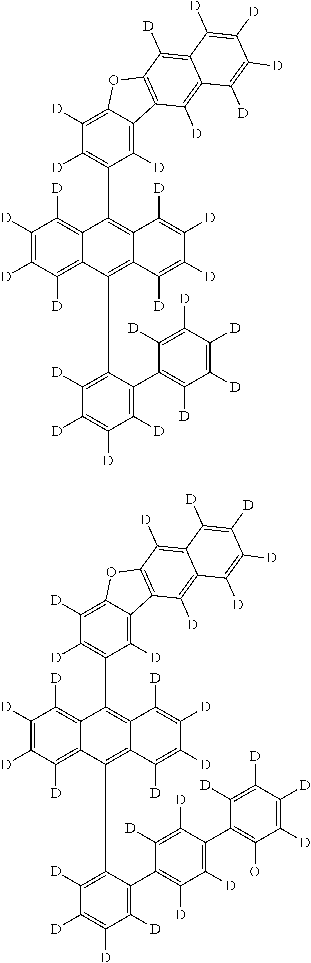 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c2[2H])c(O)c1[2H]]

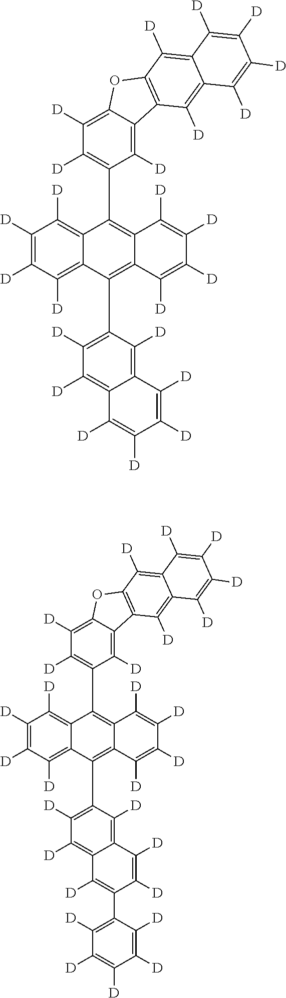 [SMILES: [2H]c1c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c([2H])c8c([2H])c([2H])c([2H])c([2H])c8c([2H])c7c6c5[2H])c5c([2H])c([2H])c([2H])c([2H])c45)c([2H])c([2H])c3c2[2H])c([2H])c1[2H]]

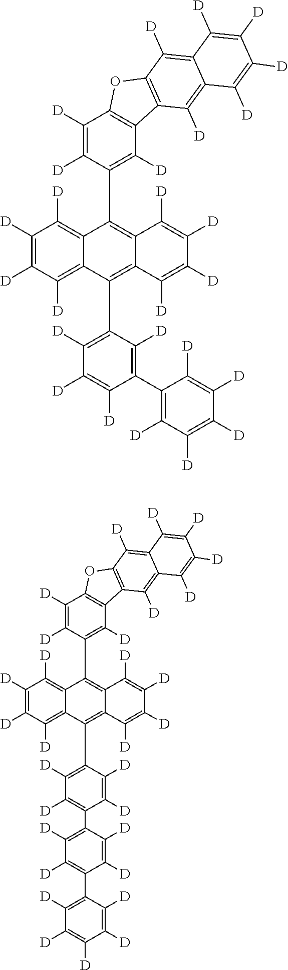 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c([2H])c8c([2H])c([2H])c([2H])c([2H])c8c([2H])c7c6c5[2H])c5c([2H])c([2H])c([2H])c([2H])c45)c([2H])c3[2H])c([2H])c2[2H])c([2H])c1[2H]]

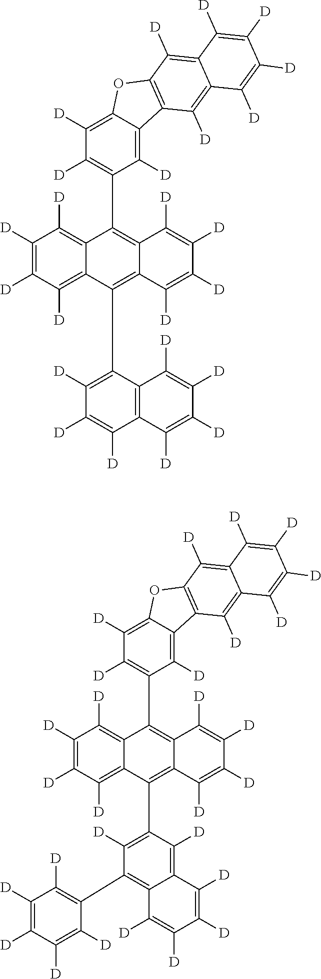 [SMILES: [2H]c1c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c3c([2H])c([2H])c([2H])c([2H])c23)c([2H])c1[2H]]

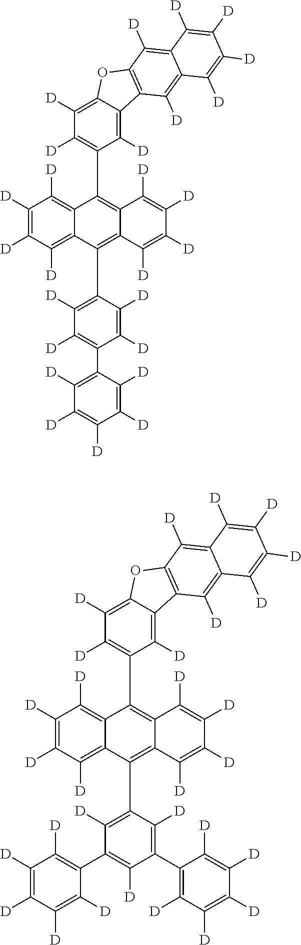 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c2[2H])c([2H])c1[2H]]

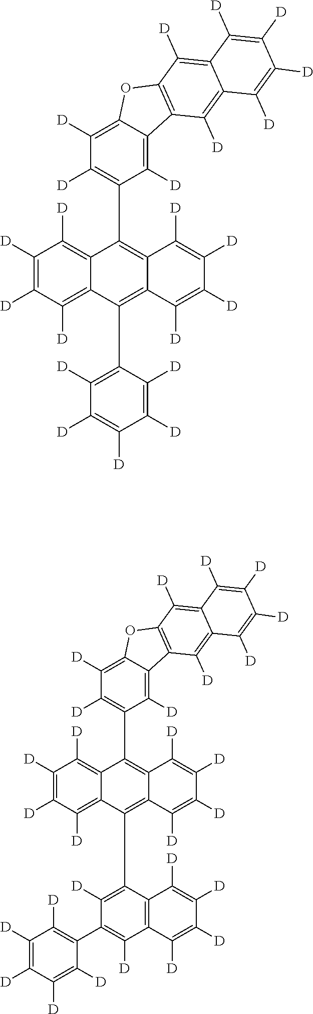 [SMILES: [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c3c([2H])c([2H])c([2H])c([2H])c3c2[2H])c([2H])c1[2H]]

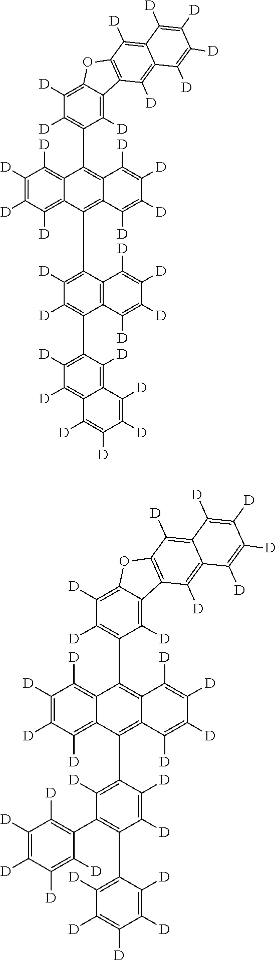

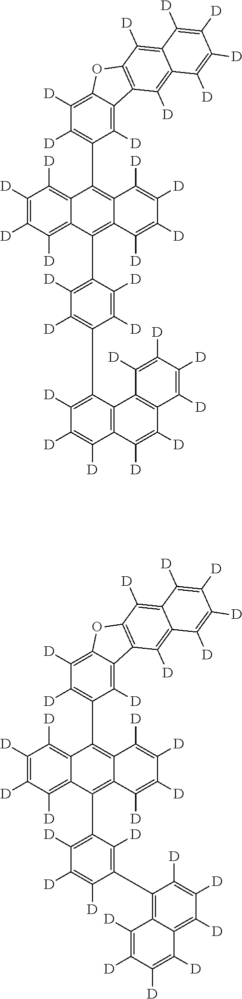 [SMILES: [2H]c1c([2H])c(-c2c([2H])c([2H])c([2H])c3c([2H])c([2H])c4c([2H])c([2H])c([2H])c([2H])c4c23)c([2H])c([2H])c1-c1c2c([2H])c([2H])c([2H])c([2H])c2c(-c2c([2H])c([2H])c3oc4c([2H])c5c([2H])c([2H])c([2H])c([2H])c5c([2H])c4c3c2[2H])c2c([2H])c([2H])c([2H])c([2H])c12 ; [2H]c1c([2H])c(-c2c([2H])c([2H])c([2H])c3c([2H])c([2H])c([2H])c([2H])c23)c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c1[2H]]

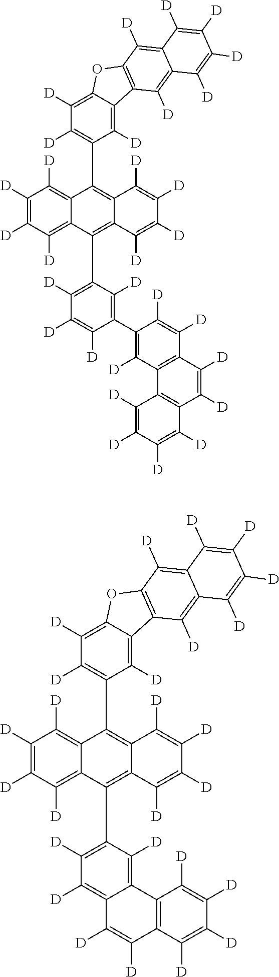 [SMILES: [2H]c1c([2H])c(-c2c([2H])c([2H])c3c([2H])c([2H])c4c([2H])c([2H])c([2H])c([2H])c4c3c2[2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c1[2H] ; [2H]c1c(-c2c3c([2H])c([2H])c([2H])c([2H])c3c(-c3c([2H])c([2H])c4c([2H])c([2H])c5c([2H])c([2H])c([2H])c([2H])c5c4c3[2H])c3c([2H])c([2H])c([2H])c([2H])c23)c([2H])c2c(oc3c([2H])c4c([2H])c([2H])c([2H])c([2H])c4c([2H])c32)c1[2H]]

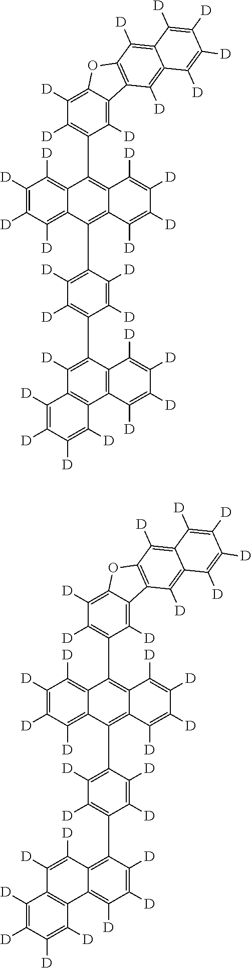

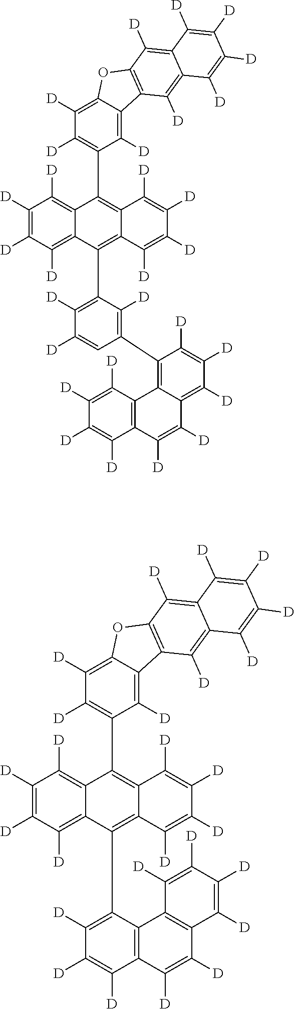 [SMILES: [2H]c1cc(-c2c([2H])c([2H])c([2H])c3c([2H])c([2H])c4c([2H])c([2H])c([2H])c([2H])c4c23)c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c1[2H] ; [2H]c1c(-c2c3c([2H])c([2H])c([2H])c([2H])c3c(-c3c([2H])c([2H])c([2H])c4c([2H])c([2H])c5c([2H])c([2H])c([2H])c([2H])c5c34)c3c([2H])c([2H])c([2H])c([2H])c23)c([2H])c2c(oc3c([2H])c4c([2H])c([2H])c([2H])c([2H])c4c([2H])c32)c1[2H]]

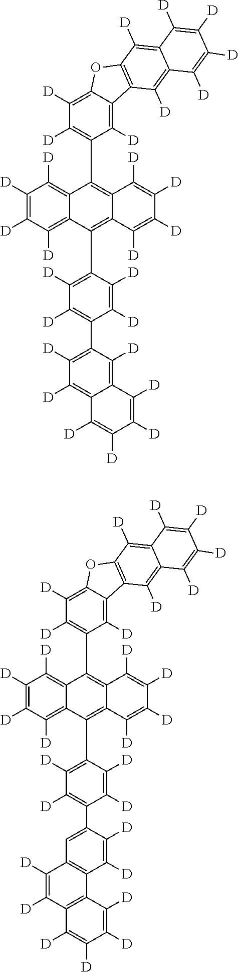 [SMILES: [2H]c1c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c([2H])c1-c1c([2H])c([2H])c2c([2H])c([2H])c([2H])c([2H])c2c1[2H] ; [2H]c1c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c([2H])c1-c1cc2c([2H])c([2H])c3c([2H])c([2H])c([2H])c([2H])c3c2c([2H])c1[2H]]

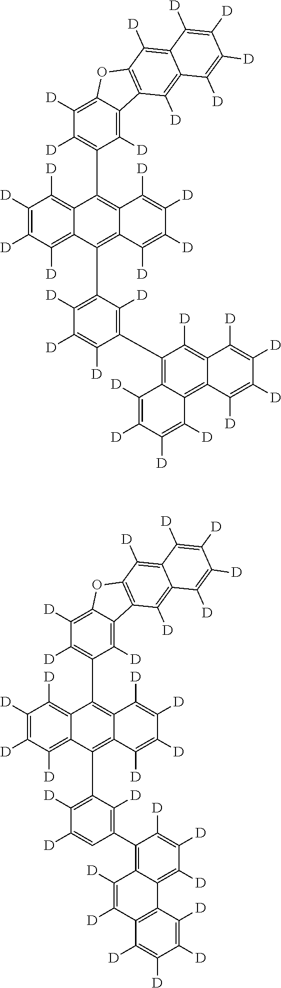

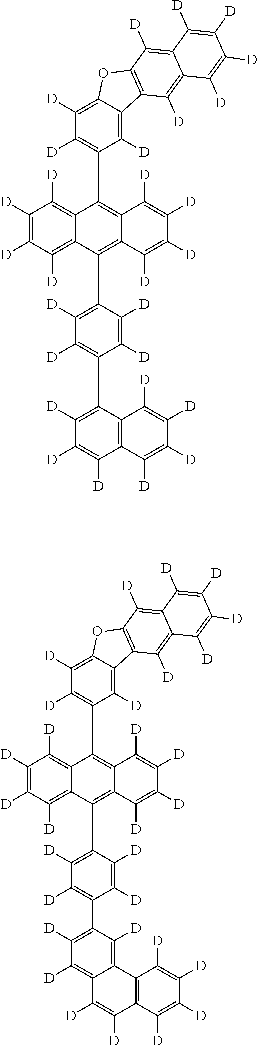 [SMILES: [2H]c1c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c([2H])c1-c1c([2H])c([2H])c([2H])c2c([2H])c([2H])c([2H])c([2H])c12 ; [2H]c1c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c([2H])c1-c1c([2H])c([2H])c2c([2H])c([2H])c3c([2H])c([2H])c([2H])c([2H])c3c2c1[2H]]

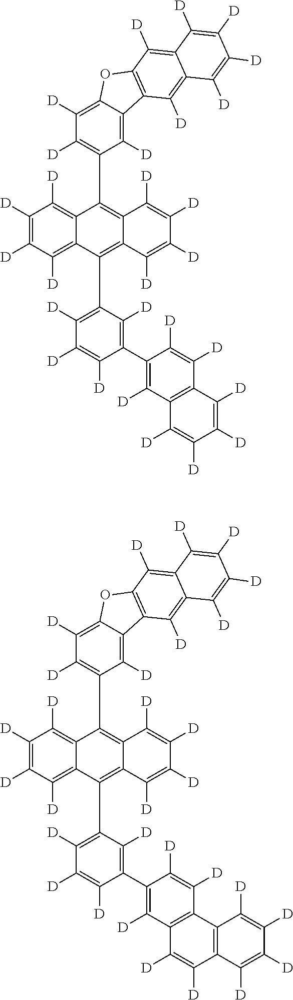 [SMILES: [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c1[2H] ; [2H]c1c([2H])c(-c2c([2H])c([2H])c3c(c2[2H])c([2H])c([2H])c2c([2H])c([2H])c([2H])c([2H])c23)c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c1[2H]]

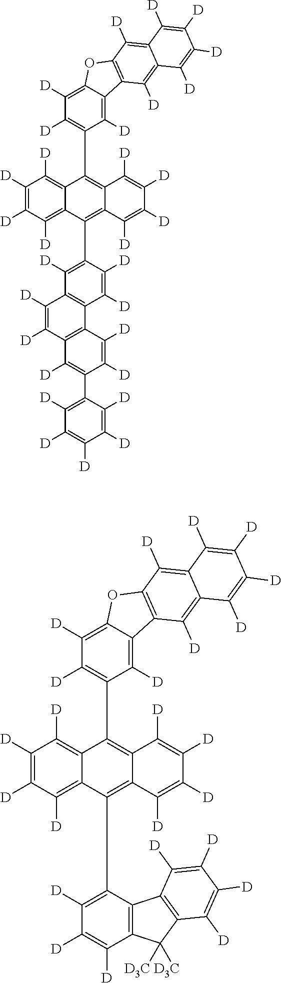

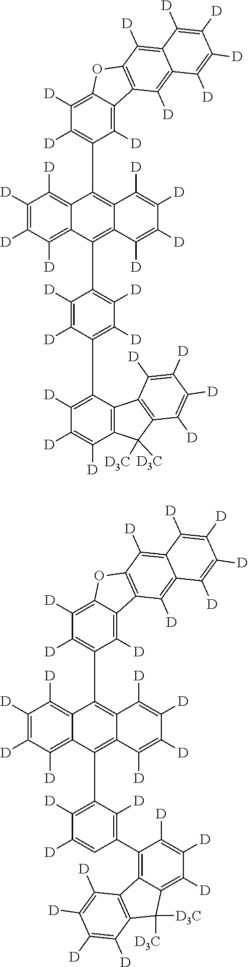

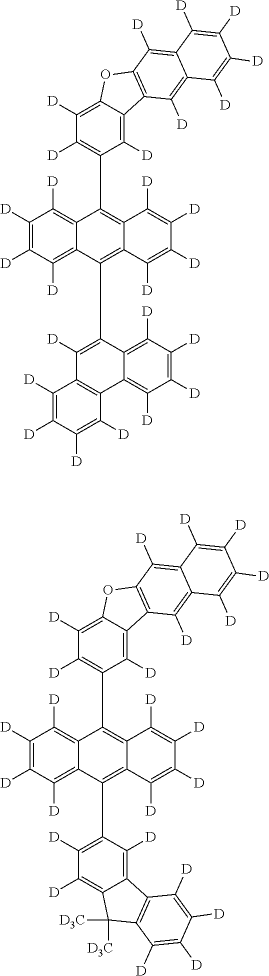 [SMILES: [2H]c1c(-c2c3c([2H])c([2H])c([2H])c([2H])c3c(-c3c([2H])c4c([2H])c([2H])c([2H])c([2H])c4c4c([2H])c([2H])c([2H])c([2H])c34)c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c2c(c1[2H])-c1c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c([2H])c1C2(C([2H])([2H])[2H])C([2H])([2H])[2H]]

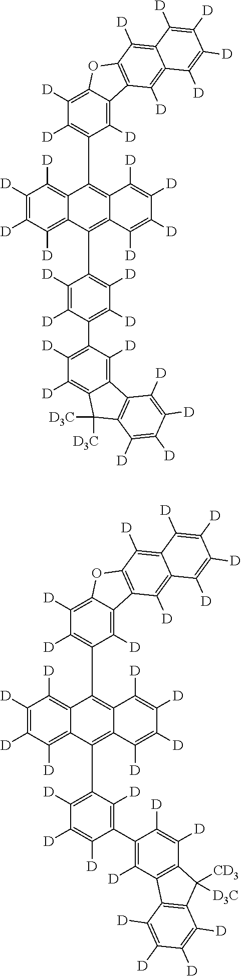

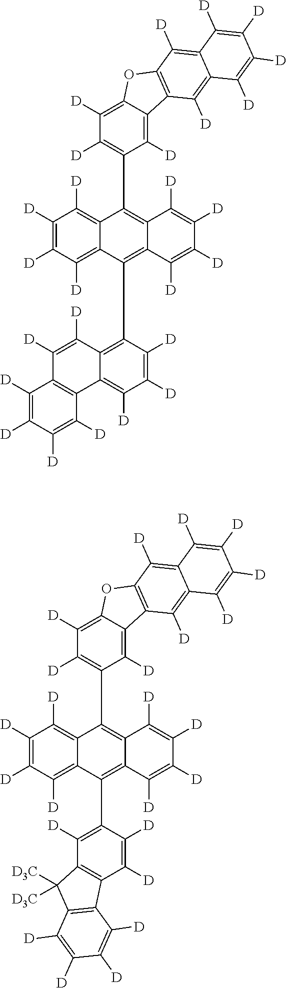 [SMILES: [2H]c1c(-c2c3c([2H])c([2H])c([2H])c([2H])c3c(-c3c([2H])c([2H])c([2H])c4c3c([2H])c([2H])c3c([2H])c([2H])c([2H])c([2H])c34)c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c2c(c1[2H])-c1c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c1C2(C([2H])([2H])[2H])C([2H])([2H])[2H]]

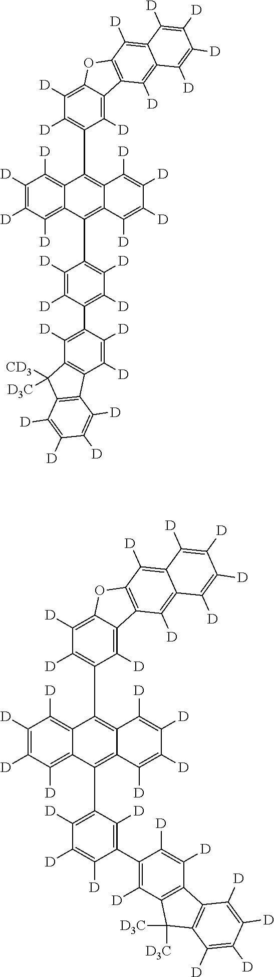

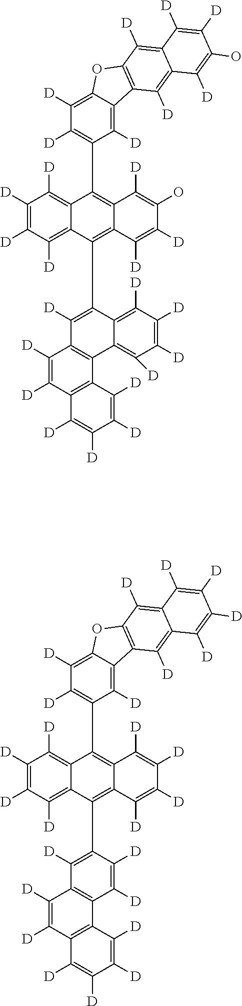 [SMILES: [2H]c1c(-c2c3c([2H])c([2H])c([2H])c([2H])c3c(-c3c([2H])c4c([2H])c([2H])c5c([2H])c([2H])c([2H])c([2H])c5c4c4c([2H])c([2H])c([2H])c([2H])c34)c3c([2H])c([2H])c(O)c([2H])c23)c([2H])c2c(oc3c([2H])c4c([2H])c([2H])c(O)c([2H])c4c([2H])c32)c1[2H] ; [2H]c1c(-c2c3c([2H])c([2H])c([2H])c([2H])c3c(-c3c([2H])c([2H])c4c(c3[2H])c([2H])c([2H])c3c([2H])c([2H])c([2H])c([2H])c34)c3c([2H])c([2H])c([2H])c([2H])c23)c([2H])c2c(oc3c([2H])c4c([2H])c([2H])c([2H])c([2H])c4c([2H])c32)c1[2H]]

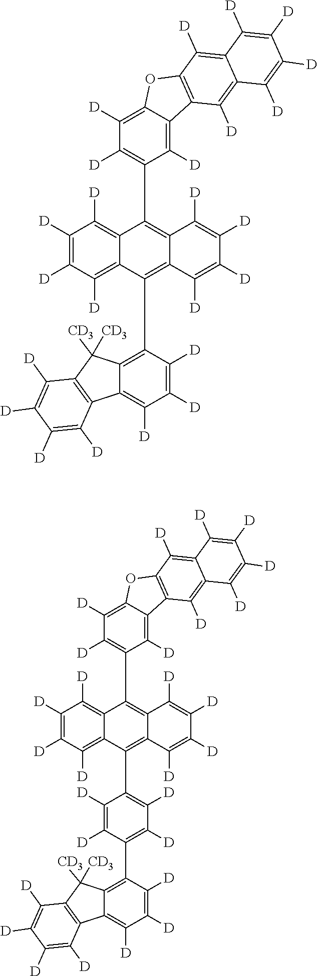

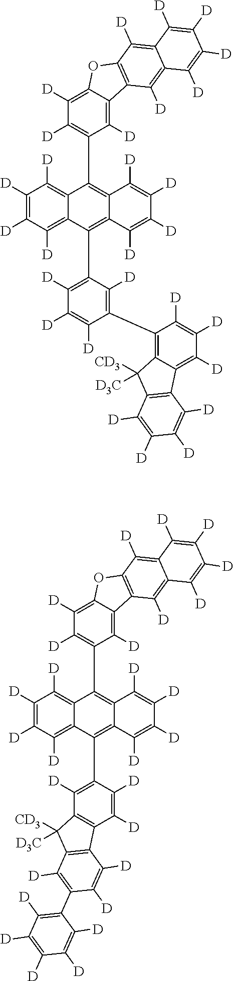

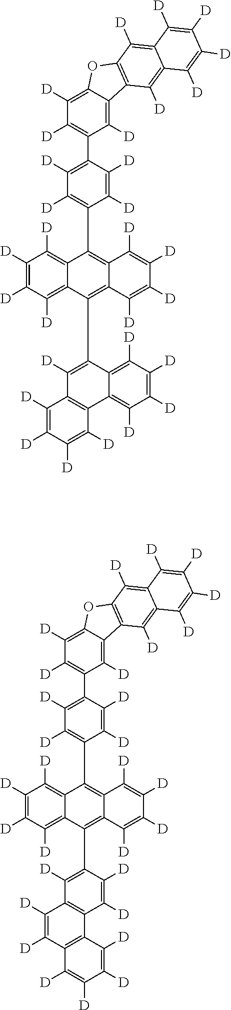

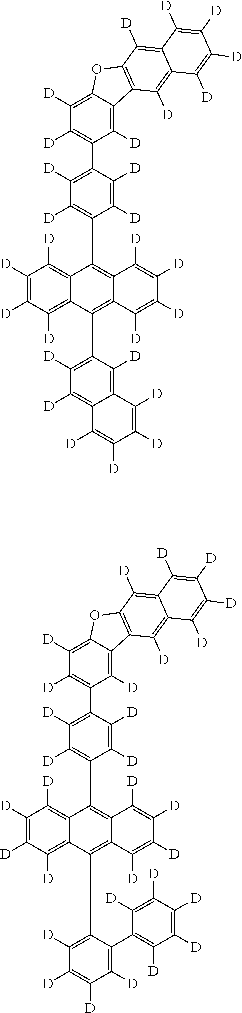 [SMILES: [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c([2H])c1-c1c([2H])c([2H])c2oc3c([2H])c4c([2H])c([2H])c([2H])c([2H])c4c([2H])c3c2c1[2H] ; [2H]c1c([2H])c([2H])c(-c2c([2H])c([2H])c([2H])c([2H])c2-c2c3c([2H])c([2H])c([2H])c([2H])c3c(-c3c([2H])c([2H])c(-c4c([2H])c([2H])c5oc6c([2H])c7c([2H])c([2H])c([2H])c([2H])c7c([2H])c6c5c4[2H])c([2H])c3[2H])c3c([2H])c([2H])c([2H])c([2H])c23)c([2H])c1[2H]]

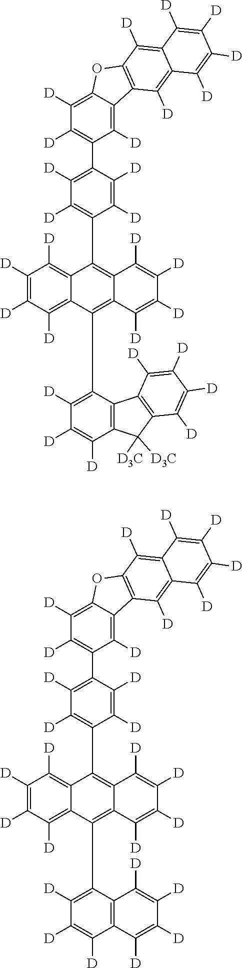

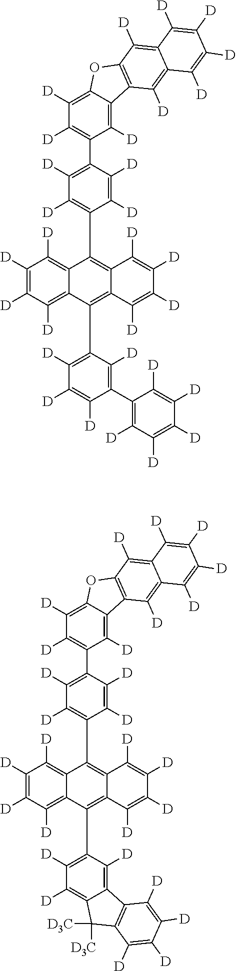

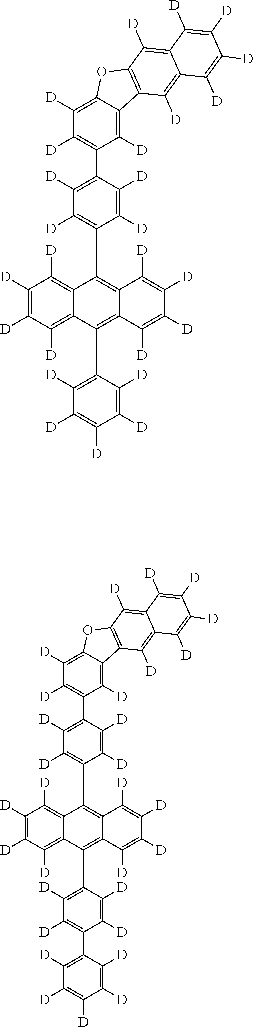 [SMILES: [2H]c1c([2H])c([2H])c(-c2c3c([2H])c([2H])c([2H])c([2H])c3c(-c3c([2H])c([2H])c(-c4c([2H])c([2H])c5oc6c([2H])c7c([2H])c([2H])c([2H])c([2H])c7c([2H])c6c5c4[2H])c([2H])c3[2H])c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4c([2H])c([2H])c(-c5c([2H])c([2H])c6oc7c([2H])c8c([2H])c([2H])c([2H])c([2H])c8c([2H])c7c6c5[2H])c([2H])c4[2H])c4c([2H])c([2H])c([2H])c([2H])c34)c([2H])c2[2H])c([2H])c1[2H]]

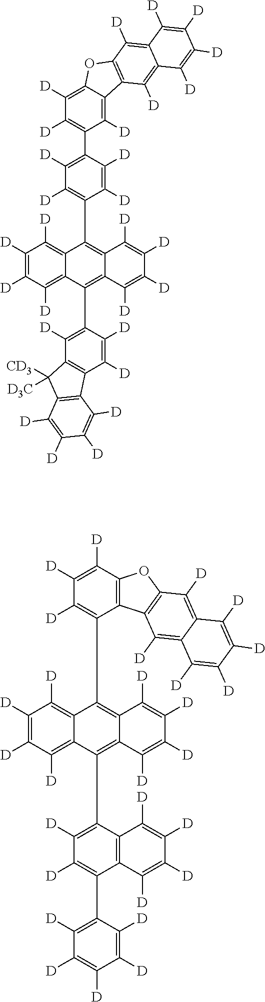

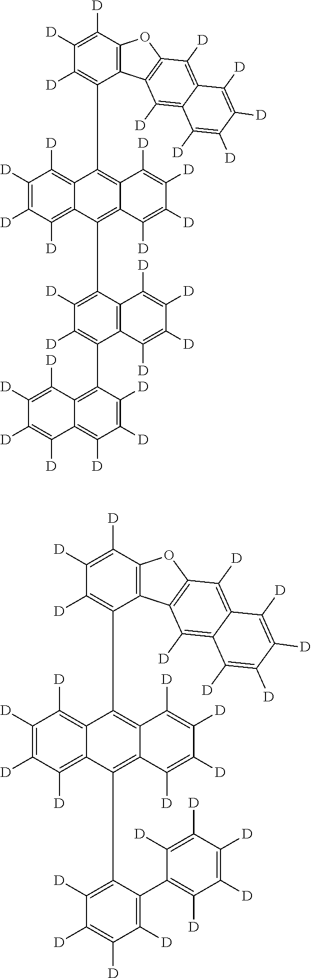 [SMILES: [2H]c1c([2H])c(-c2c3c([2H])c([2H])c([2H])c([2H])c3c(-c3c([2H])c([2H])c(-c4c([2H])c([2H])c([2H])c5c([2H])c([2H])c([2H])c([2H])c45)c4c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H]]

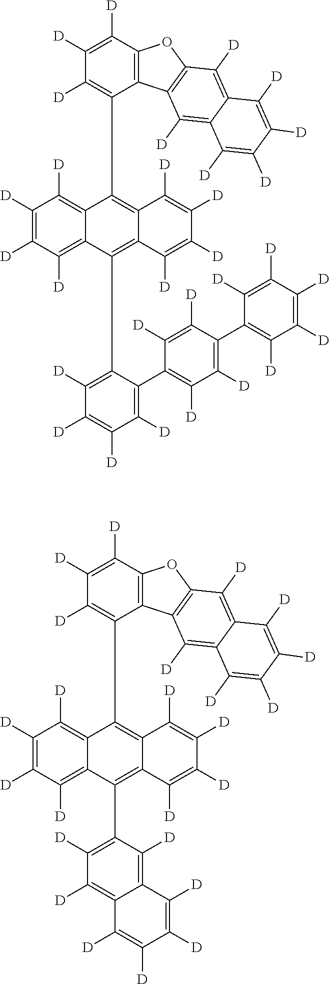 [SMILES: [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H] ; [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c2c(oc3c([2H])c4c([2H])c([2H])c([2H])c([2H])c4c([2H])c32)c1[2H]]

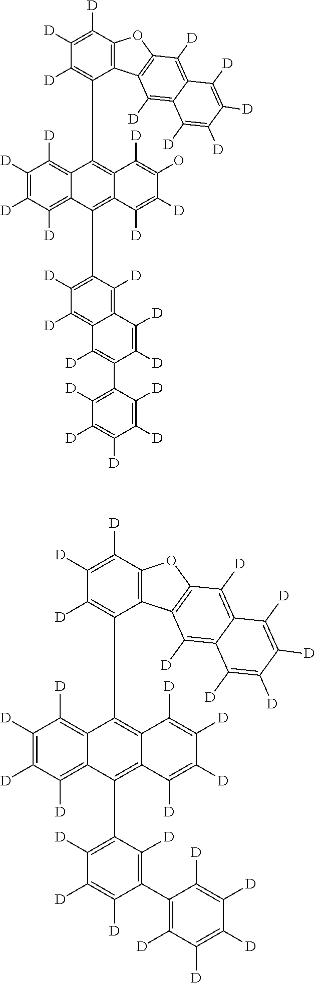 [SMILES: [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c(O)c([2H])c([2H])c45)c([2H])c([2H])c3c2[2H])c([2H])c1[2H] ; [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c2[2H])c([2H])c1[2H]]

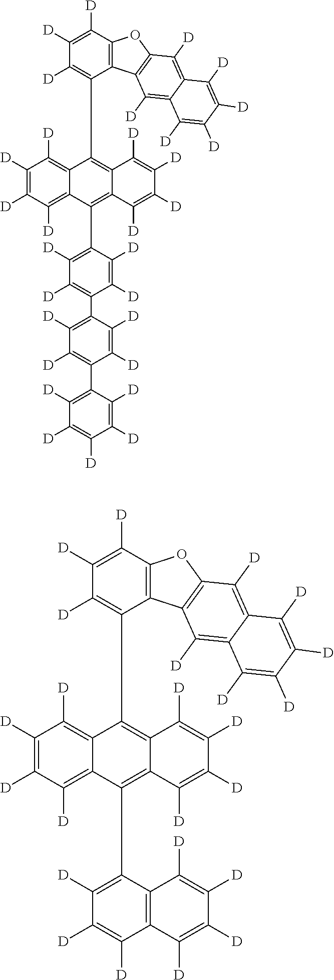 [SMILES: [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c([2H])c([2H])c([2H])c45)c([2H])c3[2H])c([2H])c2[2H])c([2H])c1[2H] ; [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H]]

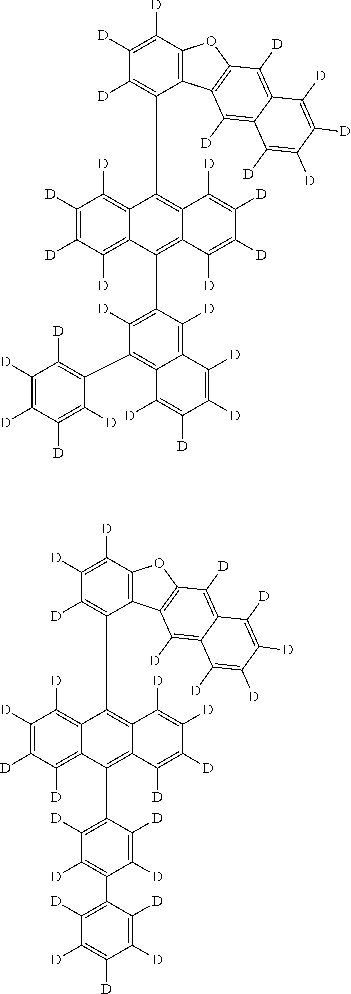 [SMILES: [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H]]

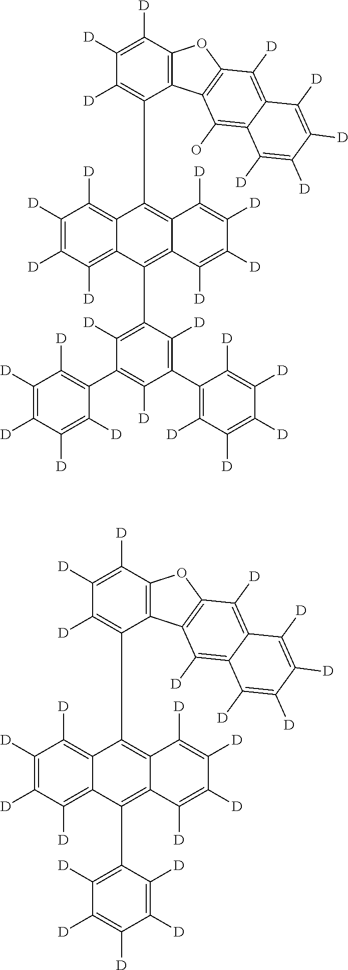 [SMILES: [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c(O)c6c45)c4c([2H])c([2H])c([2H])c([2H])c34)c2[2H])c([2H])c1[2H] ; [2H]c1c([2H])c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H]]

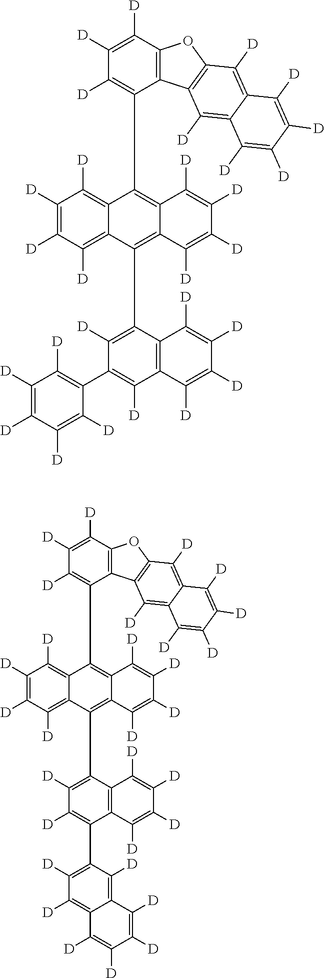 [SMILES: [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c3c([2H])c([2H])c([2H])c([2H])c3c2[2H])c([2H])c1[2H] ; [2H]c1c([2H])c(-c2c3c([2H])c([2H])c([2H])c([2H])c3c(-c3c([2H])c([2H])c(-c4c([2H])c([2H])c5c([2H])c([2H])c([2H])c([2H])c5c4[2H])c4c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H]]

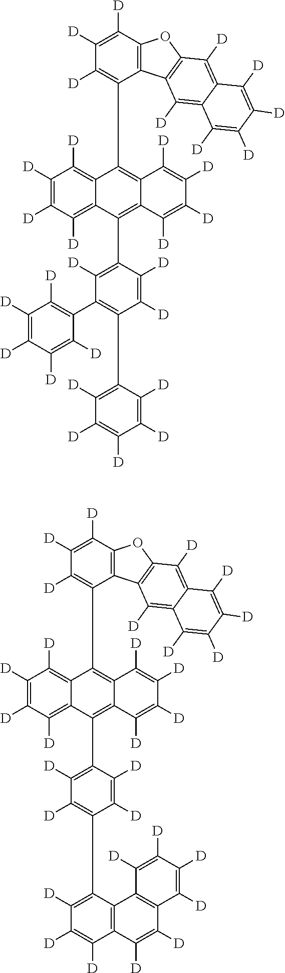

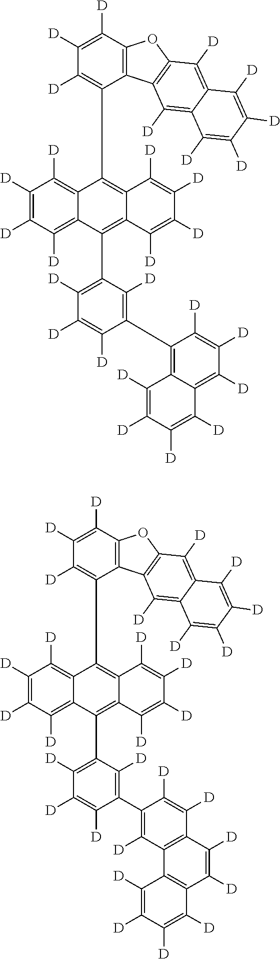 [SMILES: [2H]c1c([2H])c(-c2c([2H])c([2H])c([2H])c3c([2H])c([2H])c([2H])c([2H])c23)c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c1[2H] ; [2H]c1c([2H])c(-c2c([2H])c([2H])c3c([2H])c([2H])c4c([2H])c([2H])c([2H])c([2H])c4c3c2[2H])c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c1[2H]]

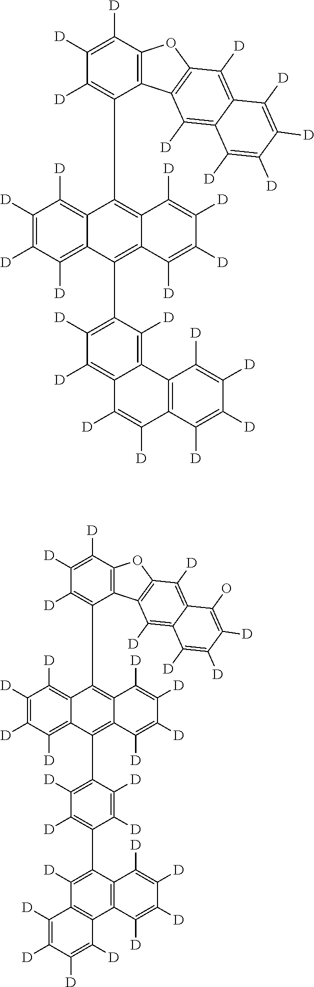 [SMILES: [2H]c1c([2H])c(-c2c3c([2H])c([2H])c([2H])c([2H])c3c(-c3c([2H])c([2H])c4c([2H])c([2H])c5c([2H])c([2H])c([2H])c([2H])c5c4c3[2H])c3c([2H])c([2H])c([2H])c([2H])c23)c2c(oc3c([2H])c4c([2H])c([2H])c([2H])c([2H])c4c([2H])c32)c1[2H] ; [2H]c1c([2H])c(-c2c([2H])c3c([2H])c([2H])c([2H])c([2H])c3c3c([2H])c([2H])c([2H])c([2H])c23)c([2H])c([2H])c1-c1c2c([2H])c([2H])c([2H])c([2H])c2c(-c2c([2H])c([2H])c([2H])c3oc4c([2H])c5c(O)c([2H])c([2H])c([2H])c5c([2H])c4c23)c2c([2H])c([2H])c([2H])c([2H])c12]

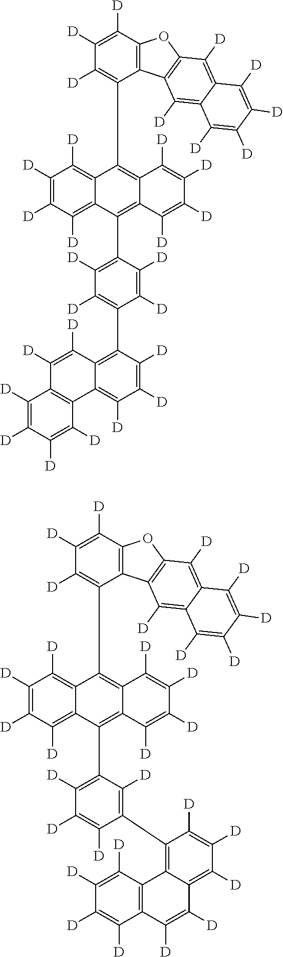

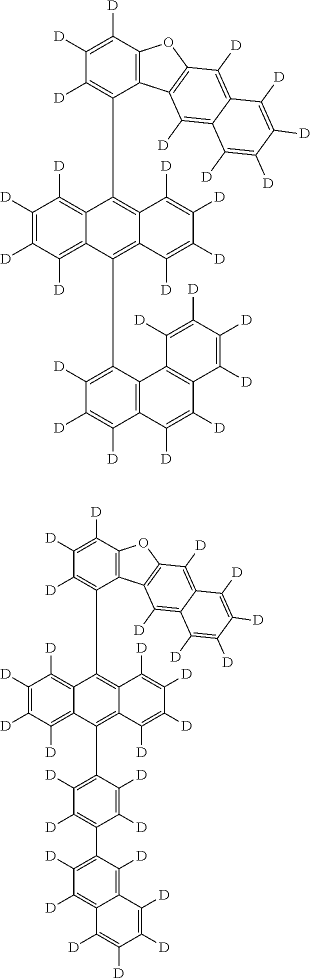 [SMILES: [2H]c1c([2H])c(-c2c3c([2H])c([2H])c([2H])c([2H])c3c(-c3c([2H])c([2H])c([2H])c4c([2H])c([2H])c5c([2H])c([2H])c([2H])c([2H])c5c34)c3c([2H])c([2H])c([2H])c([2H])c23)c2c(oc3c([2H])c4c([2H])c([2H])c([2H])c([2H])c4c([2H])c32)c1[2H] ; [2H]c1c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c([2H])c1-c1c([2H])c([2H])c2c([2H])c([2H])c([2H])c([2H])c2c1[2H]]

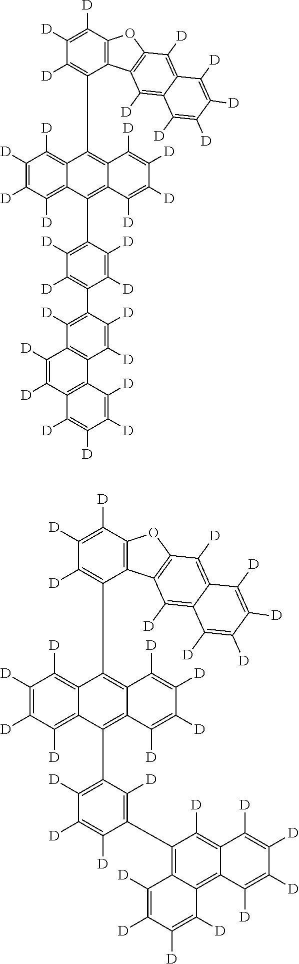

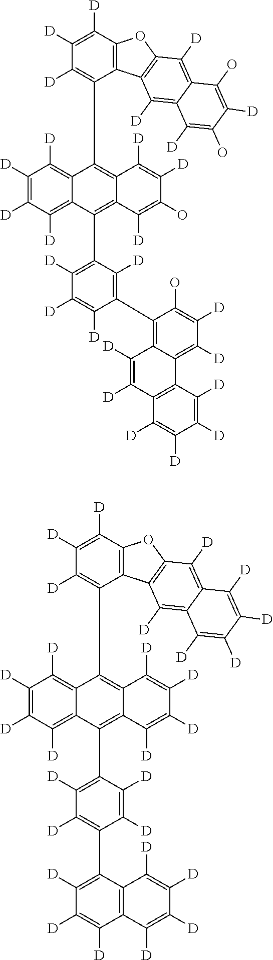 [SMILES: [2H]c1c([2H])c(-c2c3c([2H])c([2H])c([2H])c([2H])c3c(-c3c([2H])c([2H])c([2H])c4oc5c([2H])c6c(O)c([2H])c(O)c([2H])c6c([2H])c5c34)c3c([2H])c([2H])c(O)c([2H])c23)c([2H])c(-c2c(O)c([2H])c([2H])c3c2c([2H])c([2H])c2c([2H])c([2H])c([2H])c([2H])c23)c1[2H] ; [2H]c1c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c([2H])c1-c1c([2H])c([2H])c([2H])c2c([2H])c([2H])c([2H])c([2H])c12]

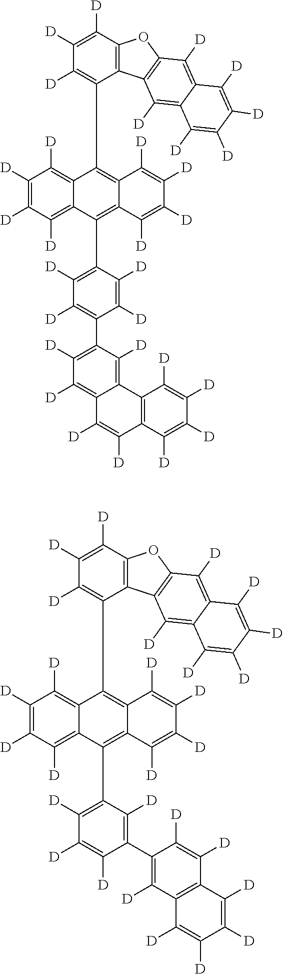 [SMILES: [2H]c1c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c([2H])c1-c1c([2H])c([2H])c2c([2H])c([2H])c3c([2H])c([2H])c([2H])c([2H])c3c2c1[2H] ; [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c1[2H]]

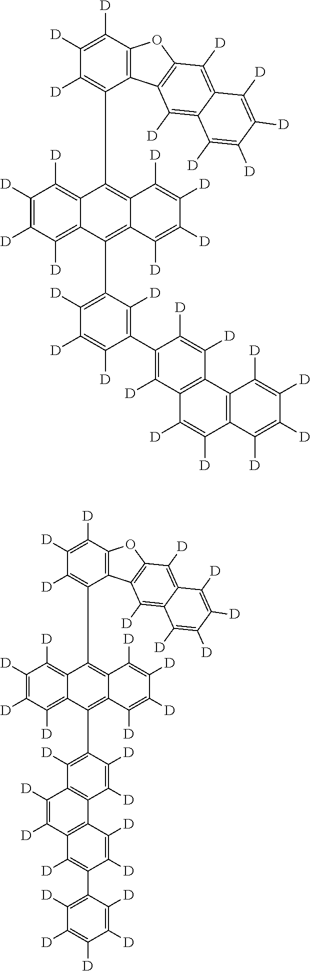

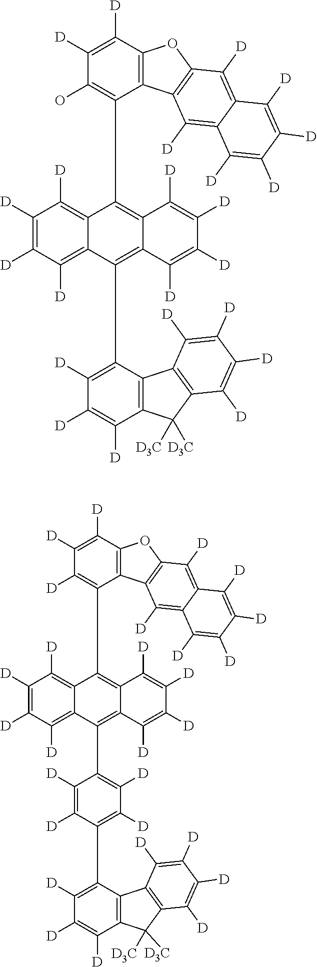

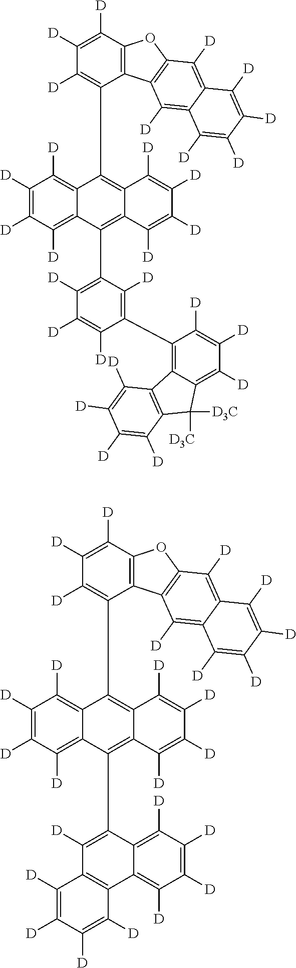

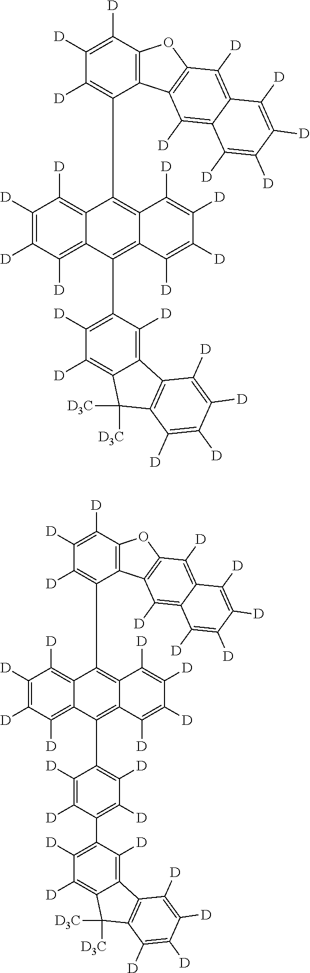

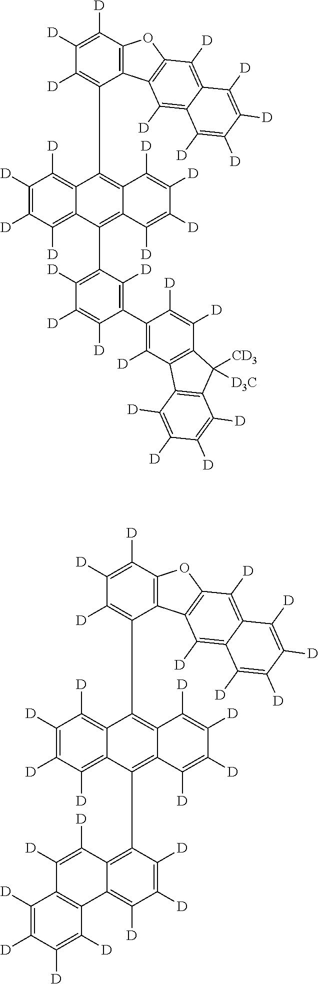

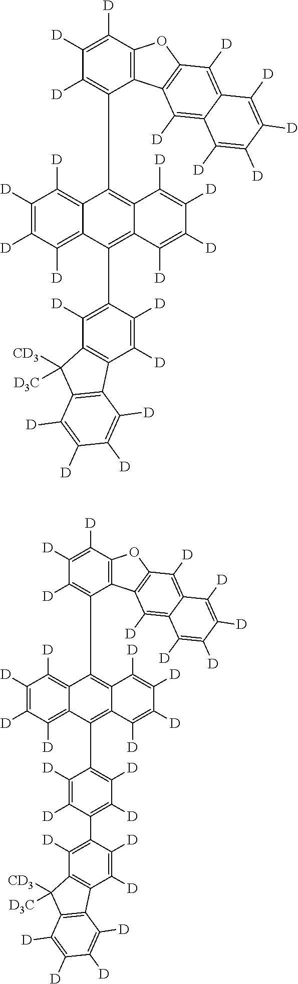

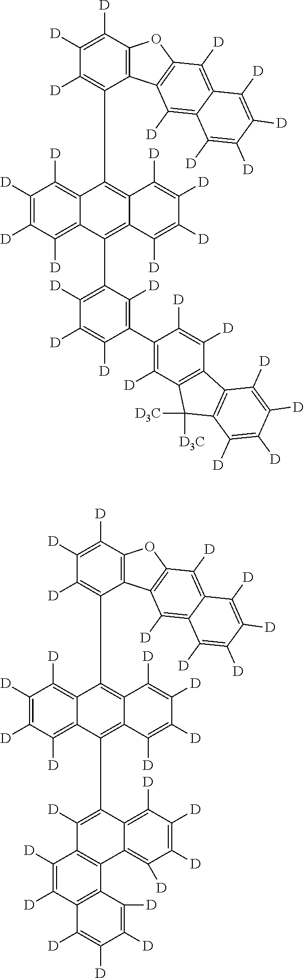

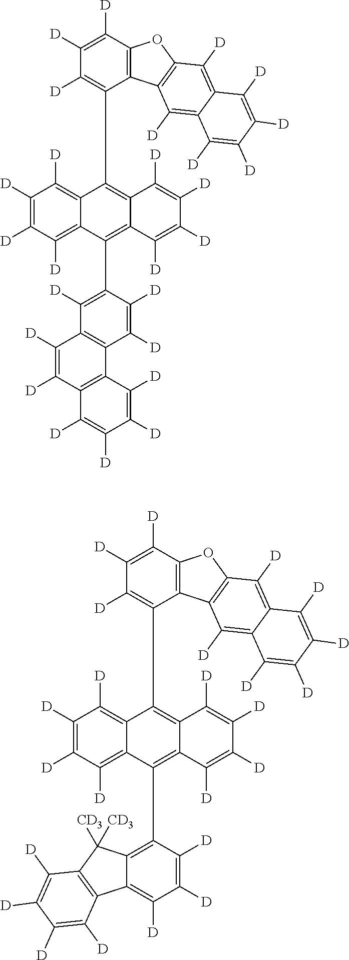 [SMILES: [2H]c1c([2H])c(-c2c3c([2H])c([2H])c([2H])c([2H])c3c(-c3c([2H])c([2H])c4c(c3[2H])c([2H])c([2H])c3c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c2c(c1[2H])-c1c([2H])c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c1C2(C([2H])([2H])[2H])C([2H])([2H])[2H]]

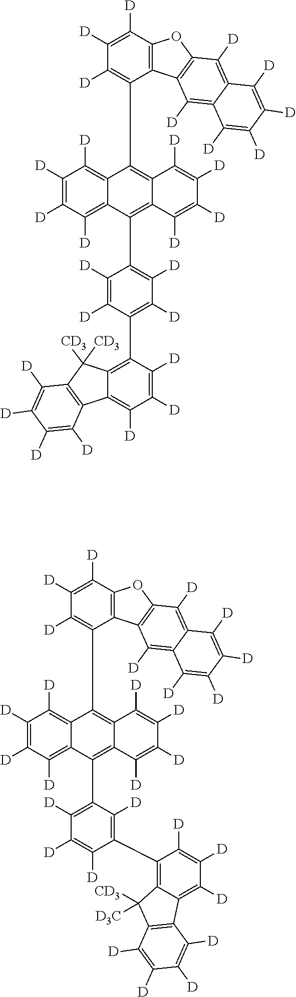

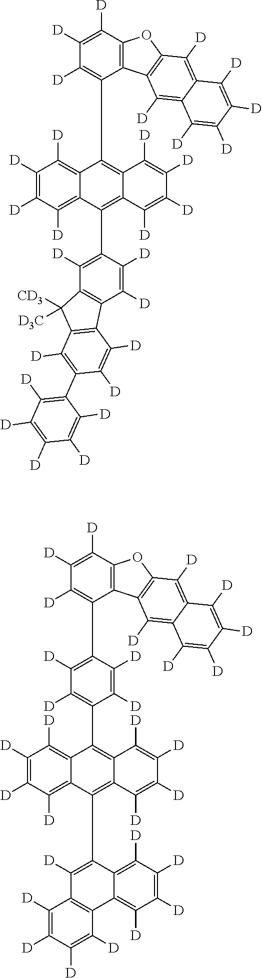

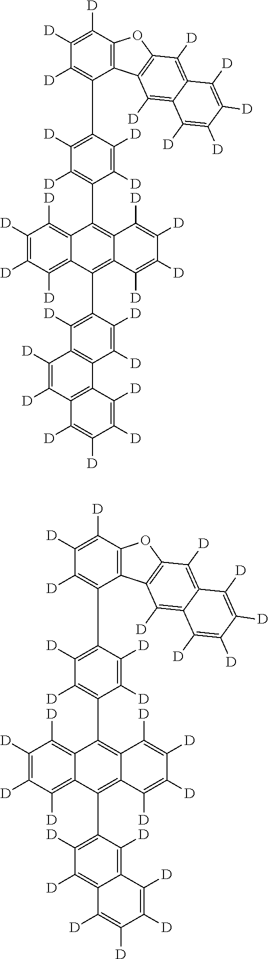 [SMILES: [2H]c1c([2H])c(-c2c([2H])c([2H])c([2H])c3oc4c([2H])c5c([2H])c([2H])c([2H])c([2H])c5c([2H])c4c23)c([2H])c([2H])c1-c1c2c([2H])c([2H])c([2H])c([2H])c2c(-c2c([2H])c([2H])c3c(c2[2H])c([2H])c([2H])c2c([2H])c([2H])c([2H])c([2H])c23)c2c([2H])c([2H])c([2H])c([2H])c12 ; [2H]c1c([2H])c(-c2c([2H])c([2H])c([2H])c3oc4c([2H])c5c([2H])c([2H])c([2H])c([2H])c5c([2H])c4c23)c([2H])c([2H])c1-c1c2c([2H])c([2H])c([2H])c([2H])c2c(-c2c([2H])c([2H])c3c([2H])c([2H])c([2H])c([2H])c3c2[2H])c2c([2H])c([2H])c([2H])c([2H])c12]

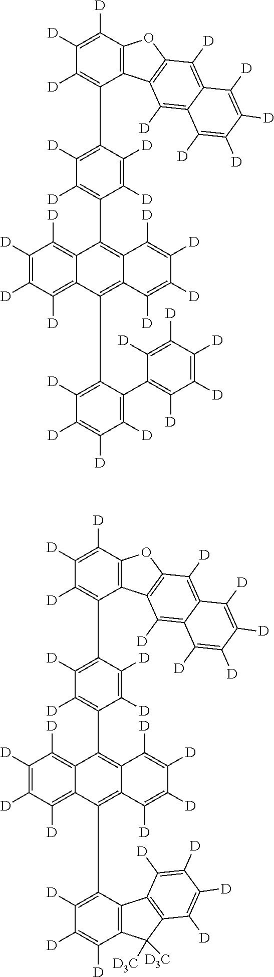

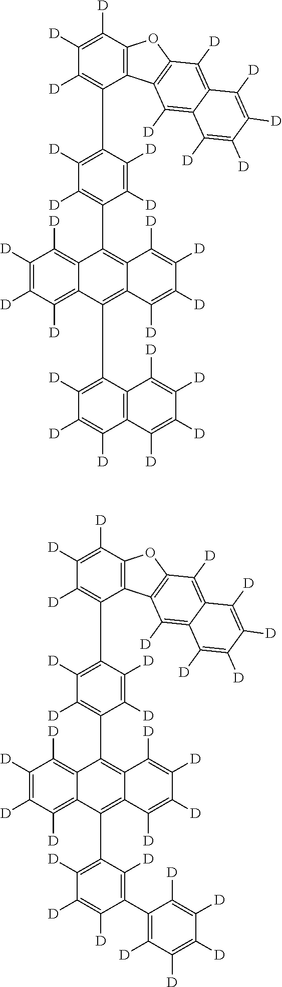 [SMILES: [2H]c1c([2H])c(-c2c([2H])c([2H])c([2H])c3oc4c([2H])c5c([2H])c([2H])c([2H])c([2H])c5c([2H])c4c23)c([2H])c([2H])c1-c1c2c([2H])c([2H])c([2H])c([2H])c2c(-c2c([2H])c([2H])c([2H])c3c([2H])c([2H])c([2H])c([2H])c23)c2c([2H])c([2H])c([2H])c([2H])c12 ; [2H]c1c([2H])c([2H])c(-c2c([2H])c([2H])c([2H])c(-c3c4c([2H])c([2H])c([2H])c([2H])c4c(-c4c([2H])c([2H])c(-c5c([2H])c([2H])c([2H])c6oc7c([2H])c8c([2H])c([2H])c([2H])c([2H])c8c([2H])c7c56)c([2H])c4[2H])c4c([2H])c([2H])c([2H])c([2H])c34)c2[2H])c([2H])c1[2H]]

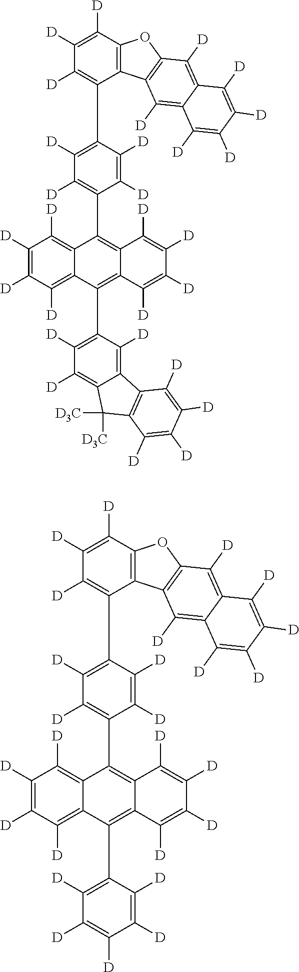

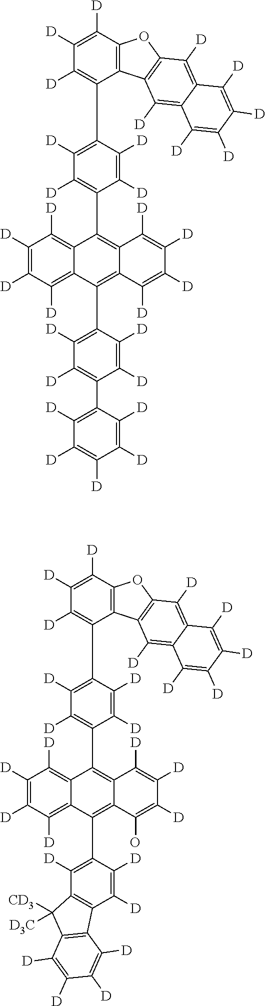

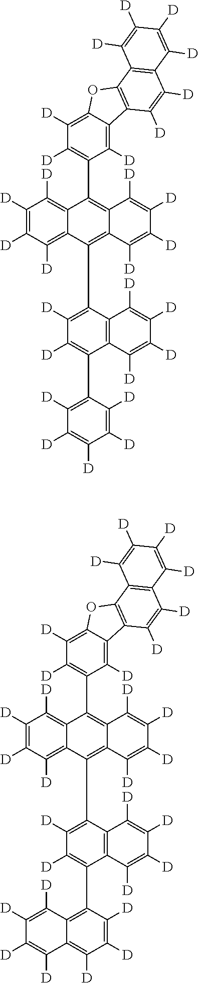

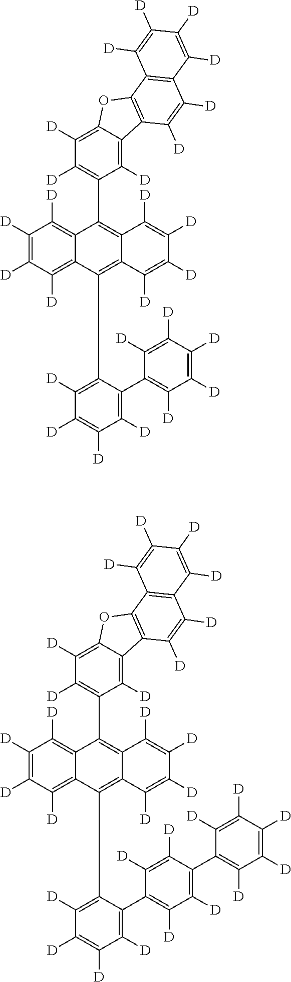 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c6c([2H])c([2H])c([2H])c([2H])c6c([2H])c([2H])c5c4c3[2H])c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c([2H])c2[2H])c([2H])c1[2H]]

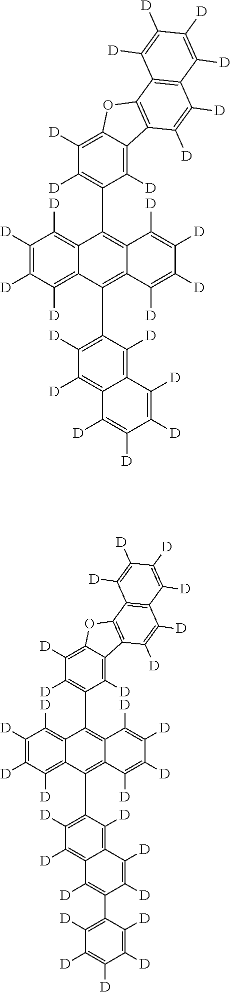 [SMILES: [2H]c1c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c2c(oc3c4c([2H])c([2H])c([2H])c([2H])c4c([2H])c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c8c([2H])c([2H])c([2H])c([2H])c8c([2H])c([2H])c7c6c5[2H])c5c([2H])c([2H])c([2H])c([2H])c45)c([2H])c([2H])c3c2[2H])c([2H])c1[2H]]

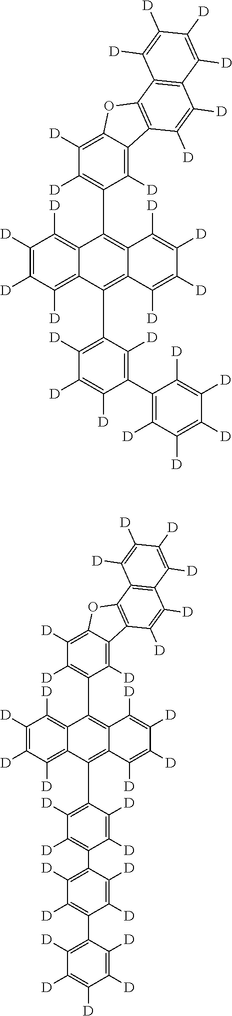 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c8c([2H])c([2H])c([2H])c([2H])c8c([2H])c([2H])c7c6c5[2H])c5c([2H])c([2H])c([2H])c([2H])c45)c([2H])c3[2H])c([2H])c2[2H])c([2H])c1[2H]]

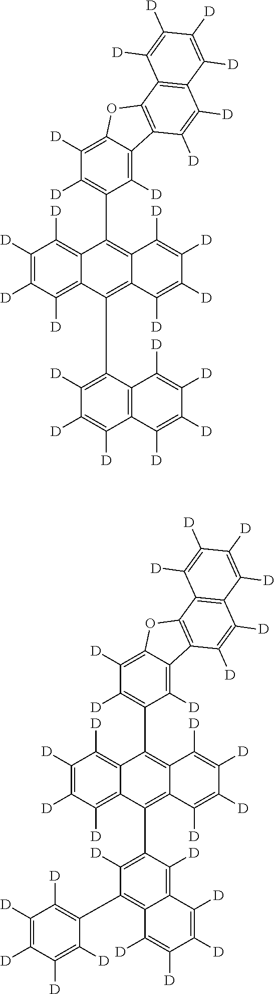 [SMILES: [2H]c1c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c2c(oc3c4c([2H])c([2H])c([2H])c([2H])c4c([2H])c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c([2H])c3c([2H])c([2H])c([2H])c([2H])c23)c([2H])c1[2H]]

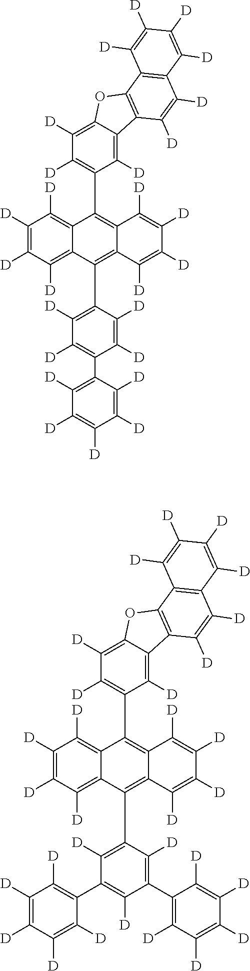 [SMILES: [2H]c1c([2H])c([2H])c(-c2c([2H])c([2H])c(-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c2[2H])c([2H])c1[2H]]

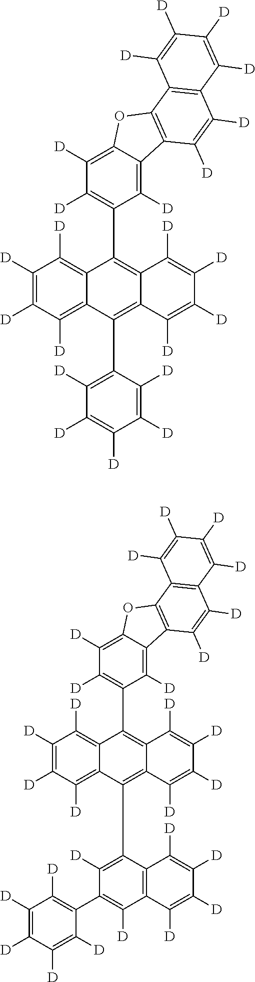 [SMILES: [2H]c1c([2H])c([2H])c(-c2c3c([2H])c([2H])c([2H])c([2H])c3c(-c3c([2H])c([2H])c4oc5c6c([2H])c([2H])c([2H])c([2H])c6c([2H])c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c7c([2H])c([2H])c([2H])c([2H])c7c([2H])c([2H])c6c5c4[2H])c4c([2H])c([2H])c([2H])c([2H])c34)c3c([2H])c([2H])c([2H])c([2H])c3c2[2H])c([2H])c1[2H]]

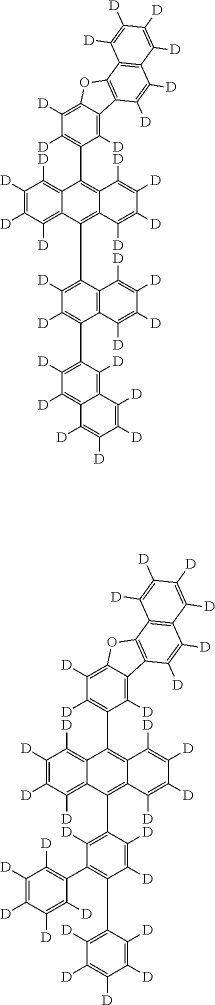

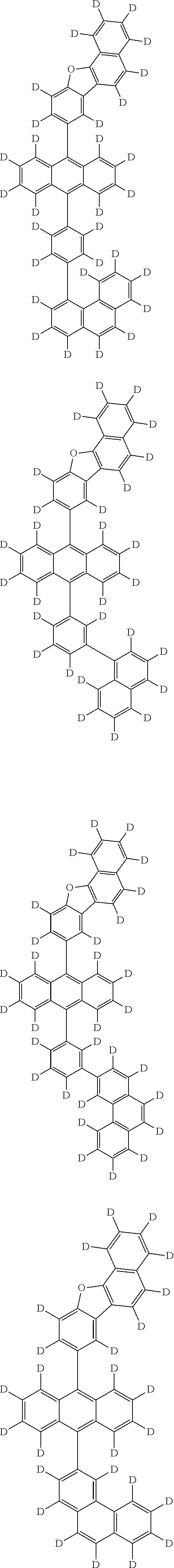

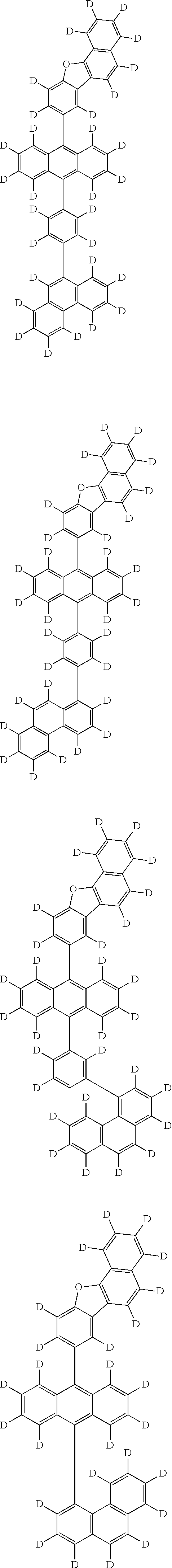

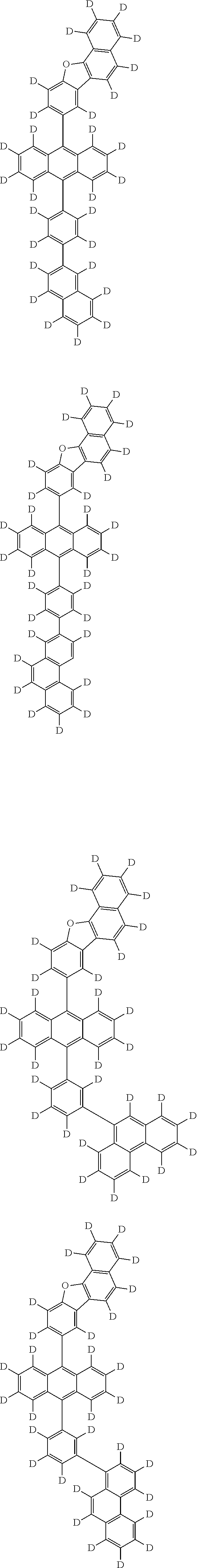

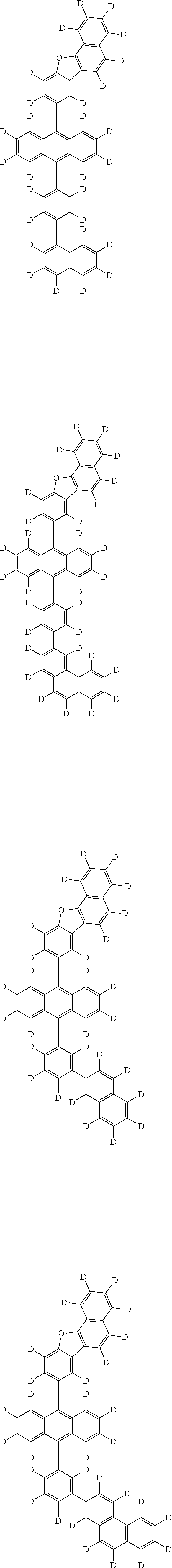

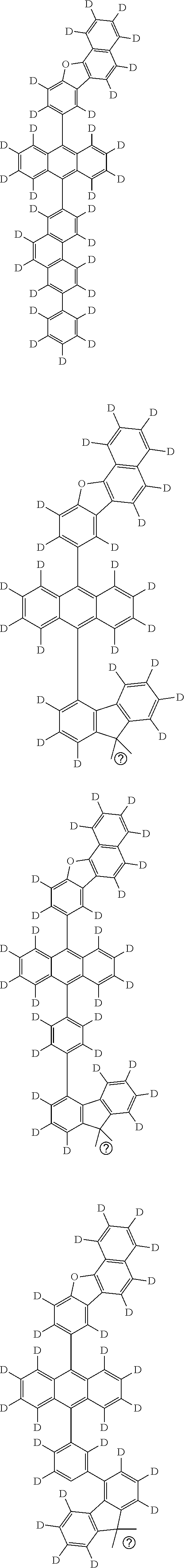

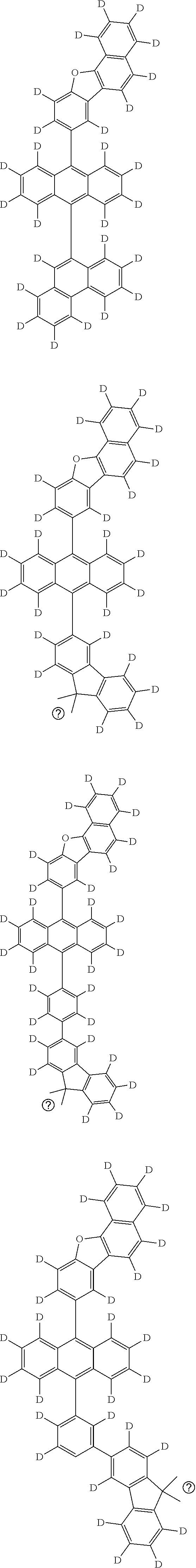

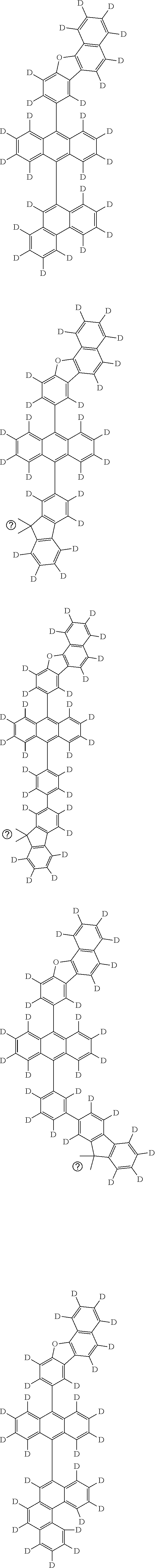

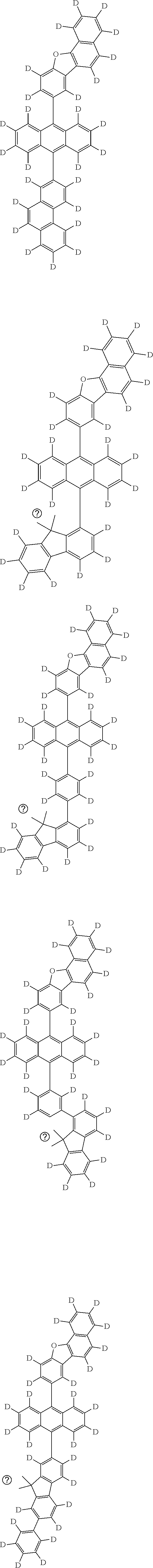

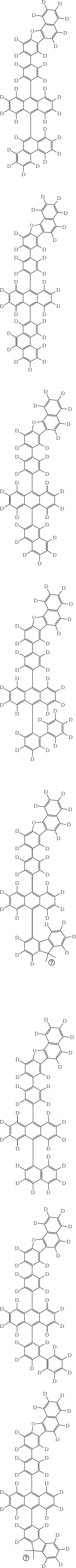

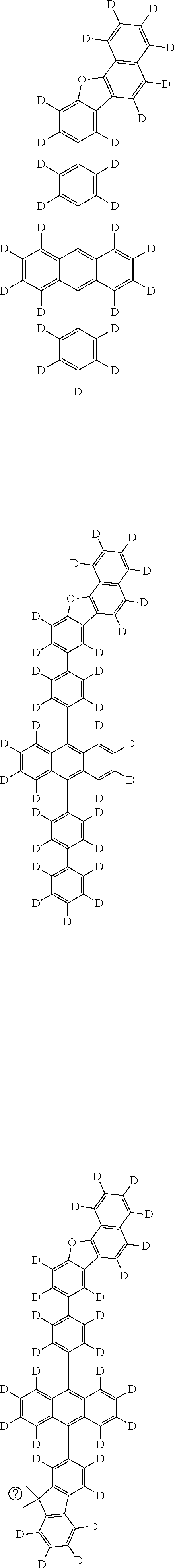

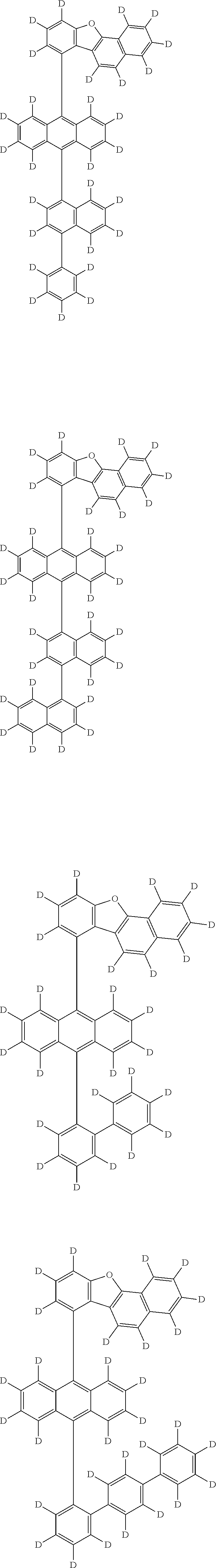

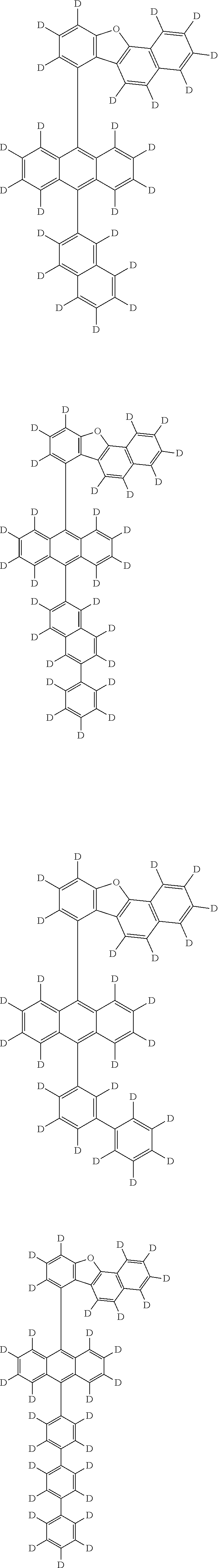

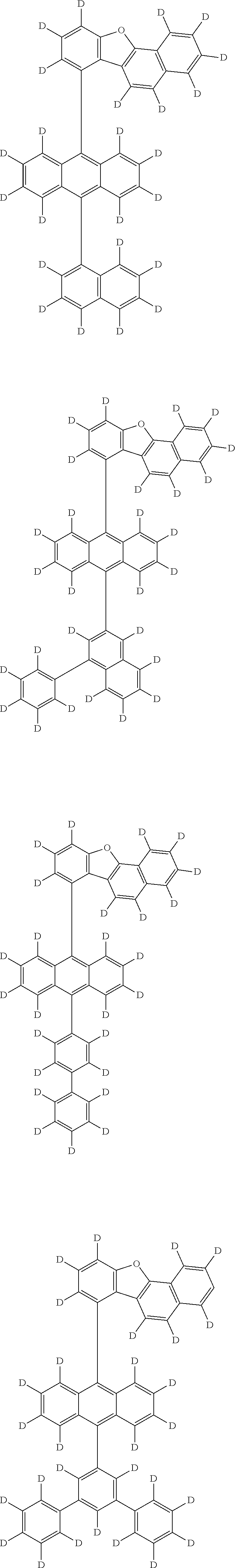

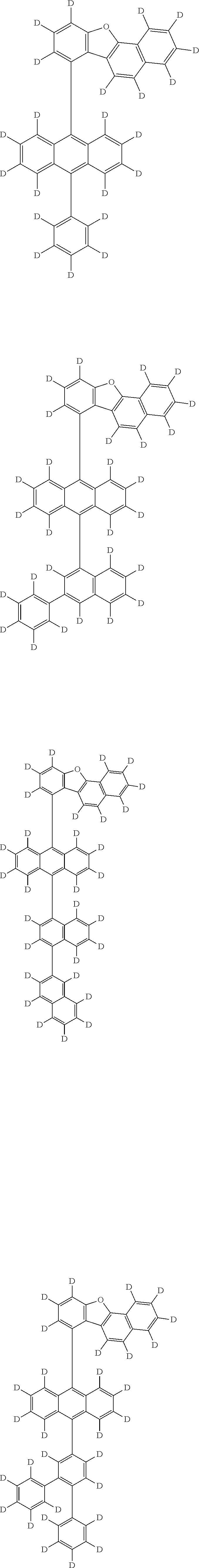

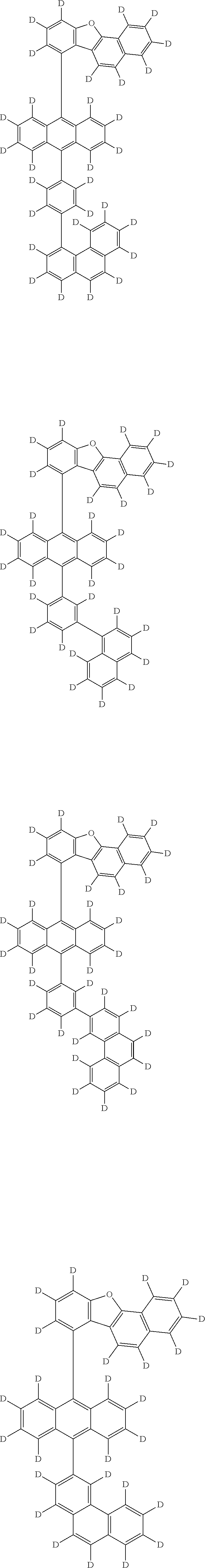

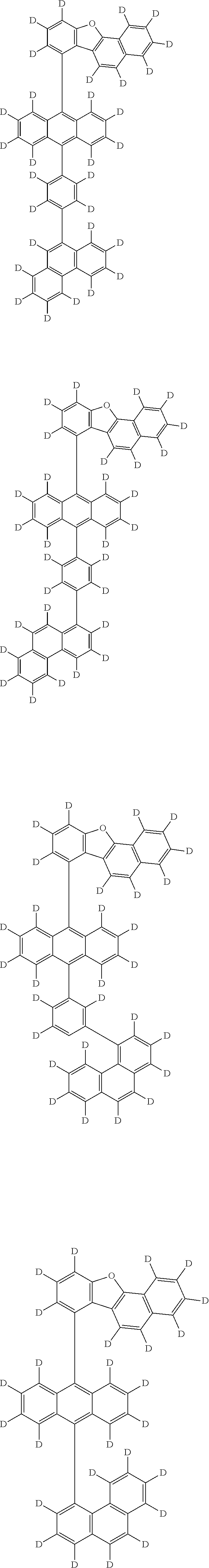

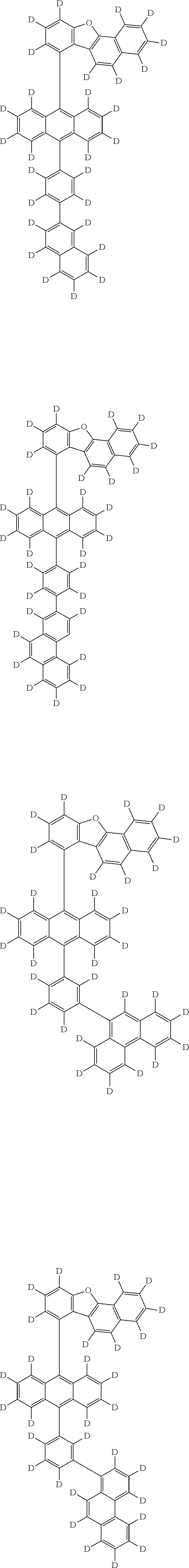

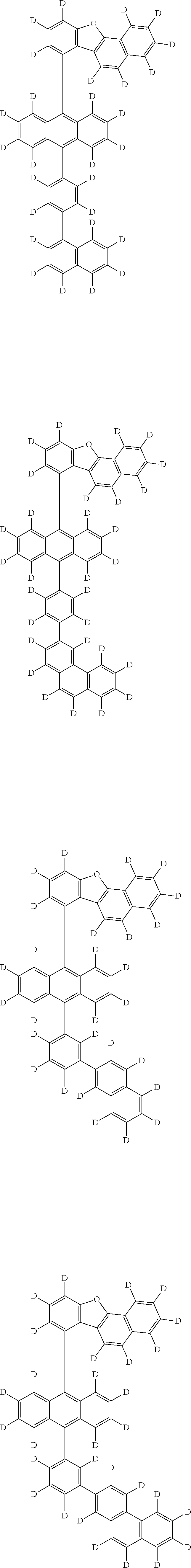

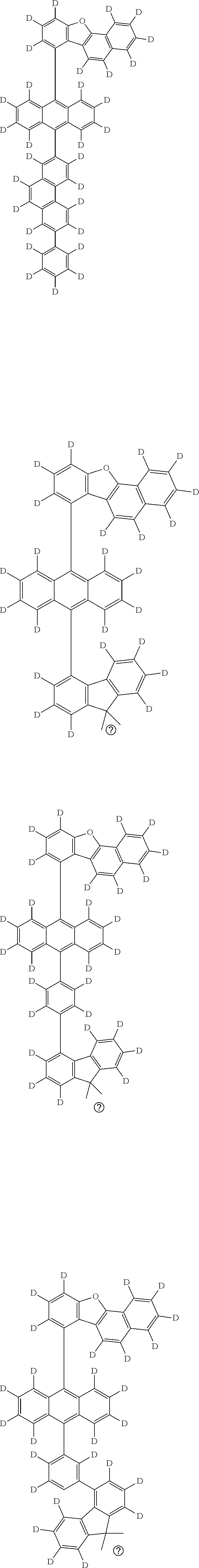

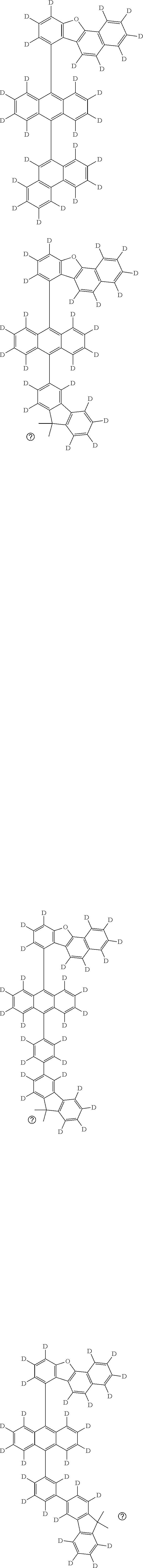

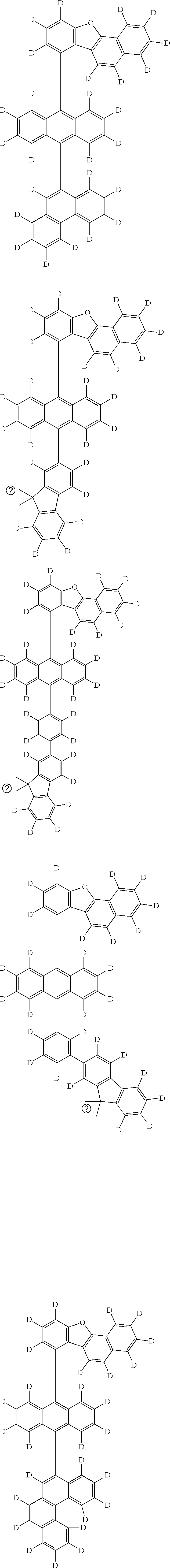

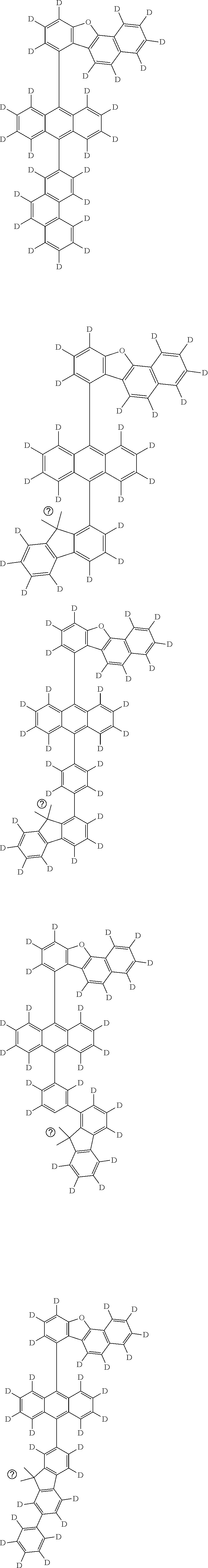

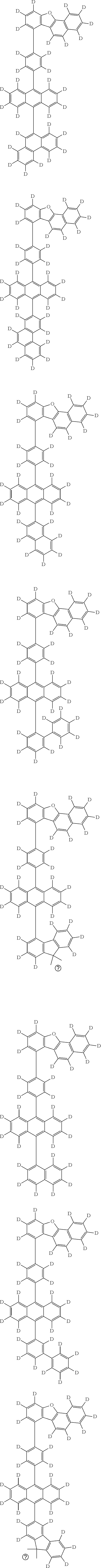

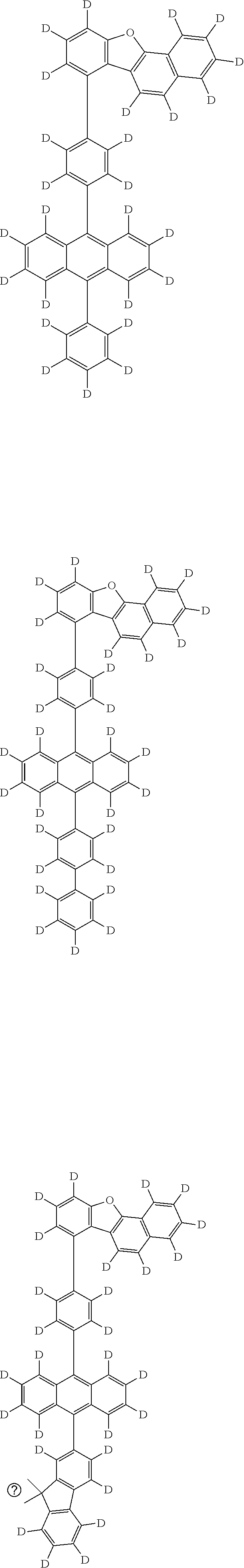

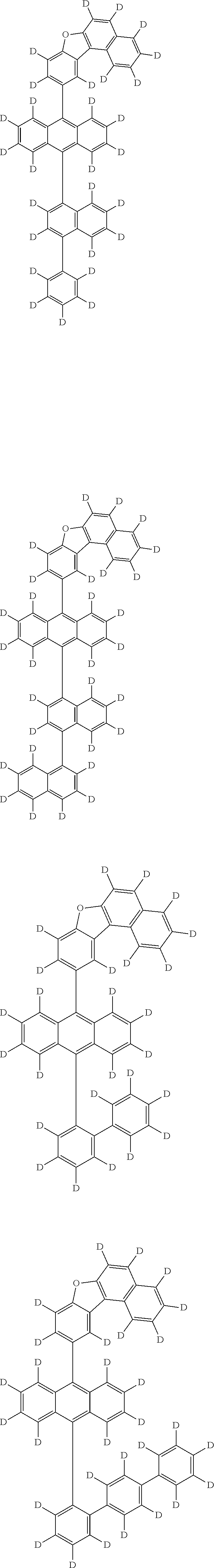

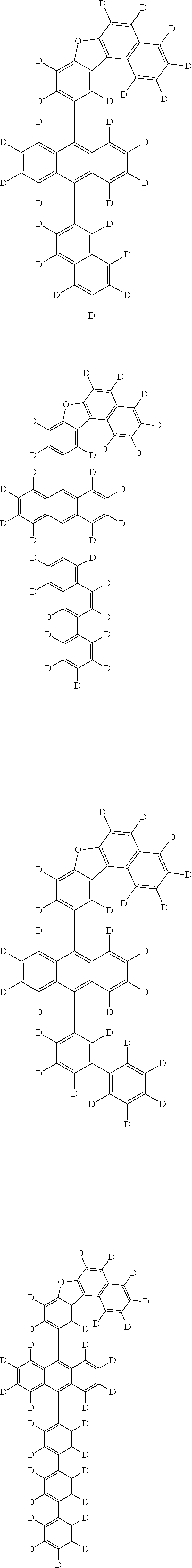

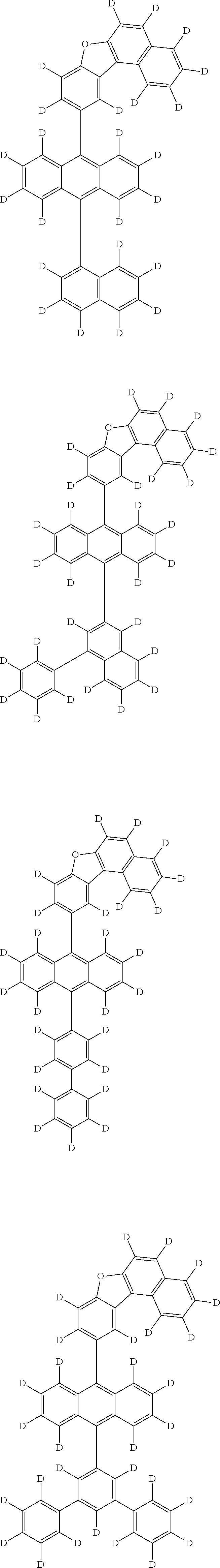

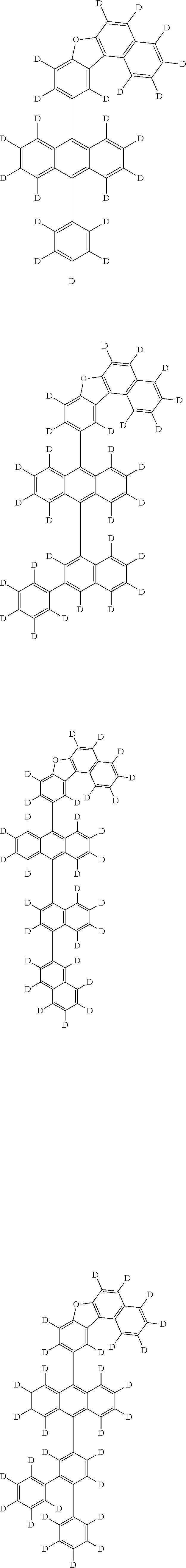

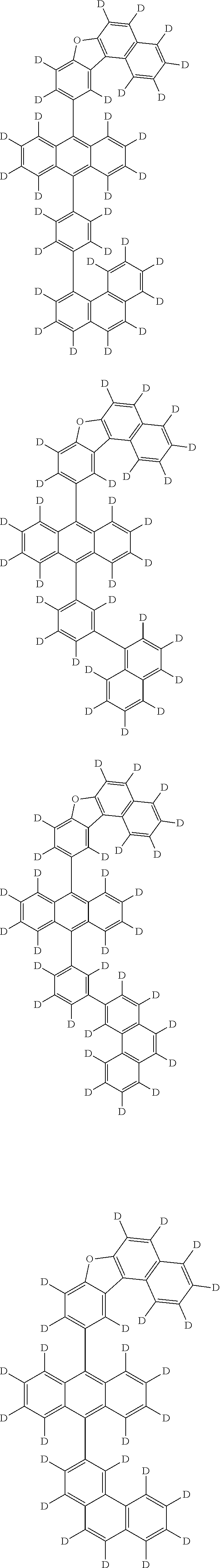

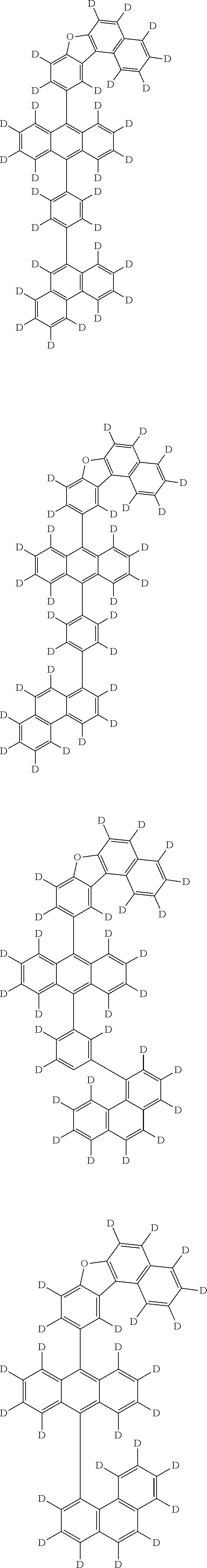

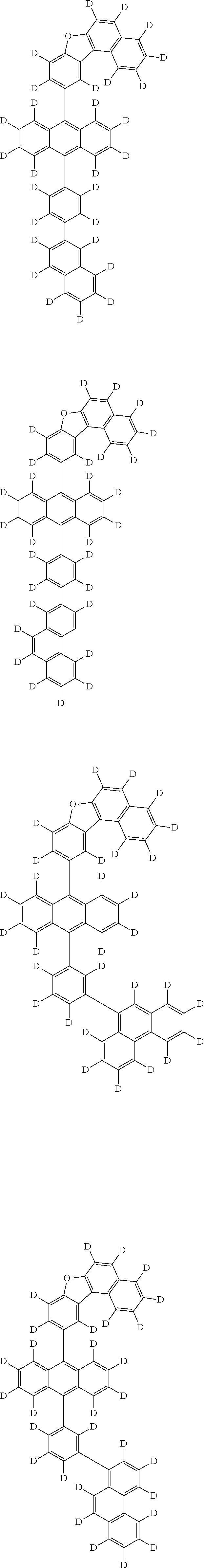

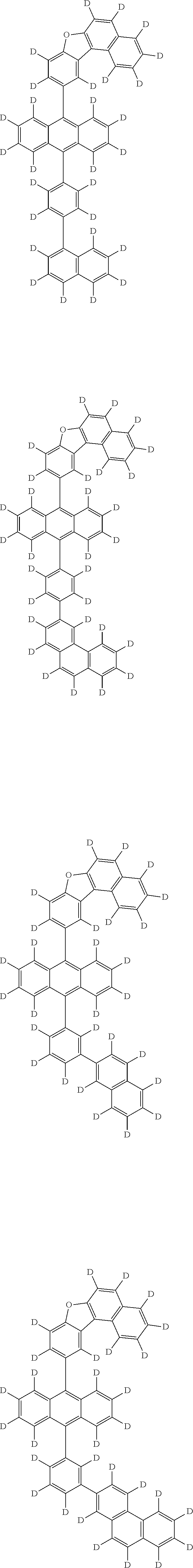

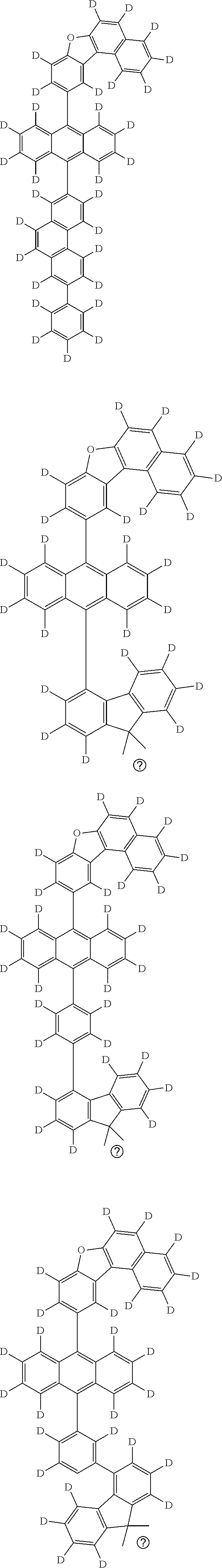

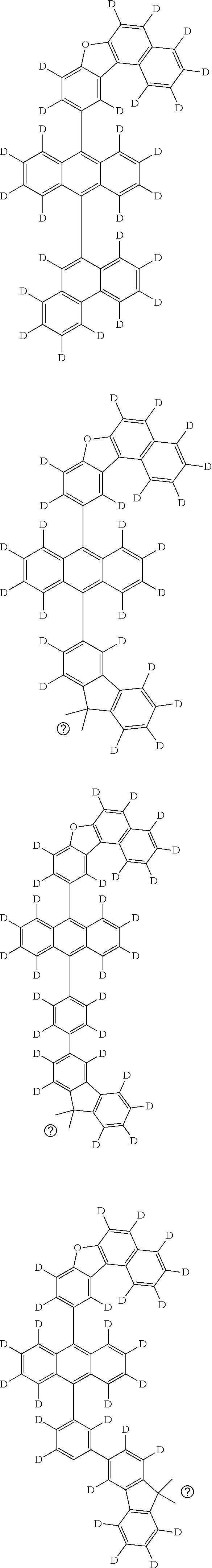

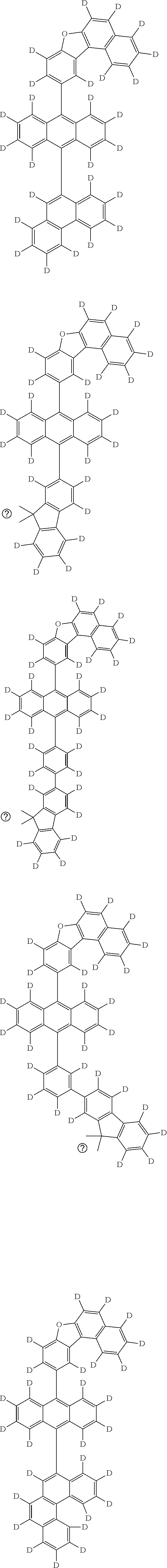

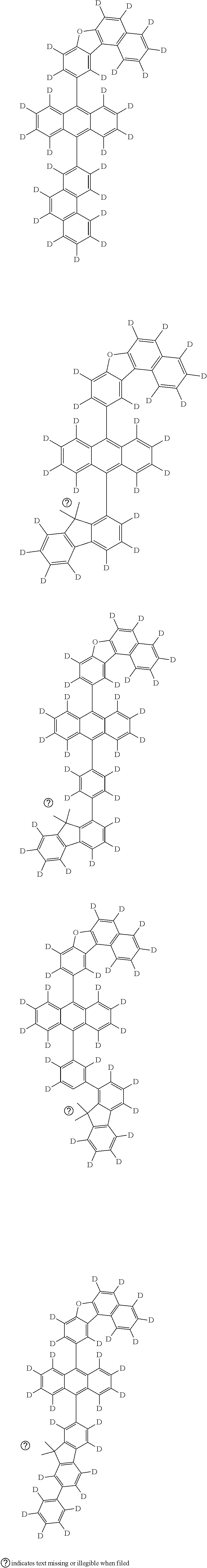

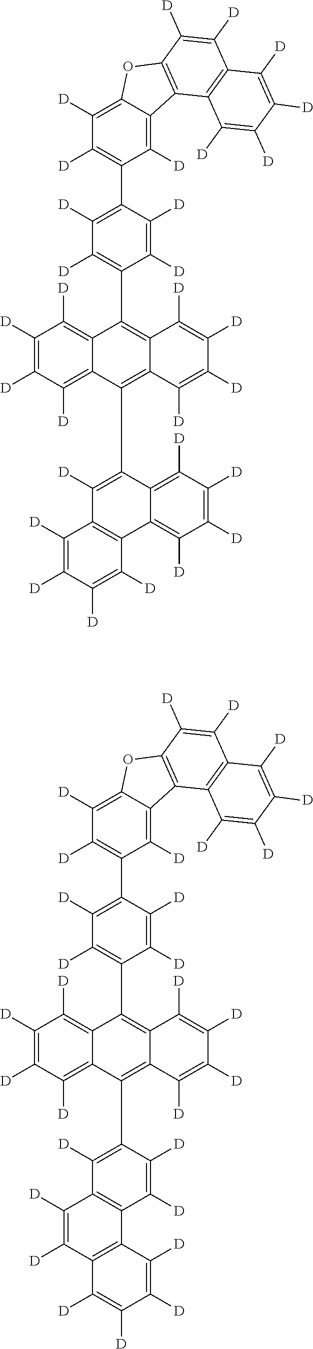

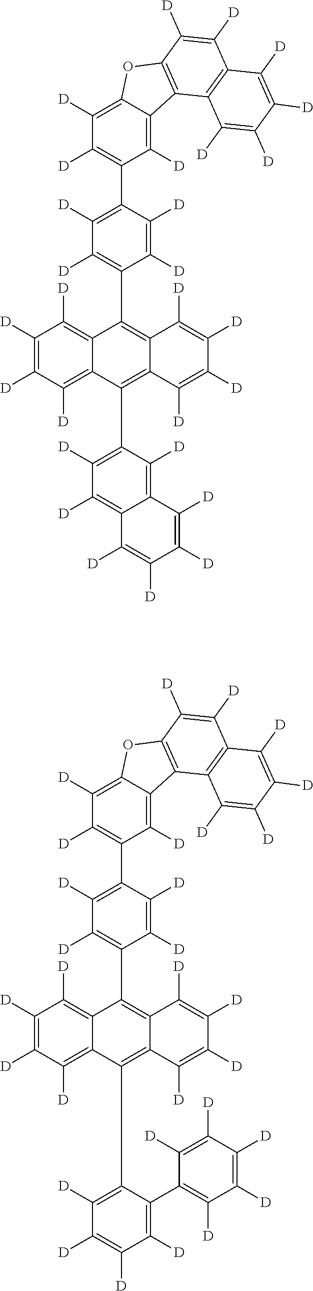 [SMILES: [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c([2H])c1-c1c([2H])c([2H])c2oc3c([2H])c([2H])c4c([2H])c([2H])c([2H])c([2H])c4c3c2c1[2H] ; [2H]c1c([2H])c([2H])c(-c2c([2H])c([2H])c([2H])c([2H])c2-c2c3c([2H])c([2H])c([2H])c([2H])c3c(-c3c([2H])c([2H])c(-c4c([2H])c([2H])c5oc6c([2H])c([2H])c7c([2H])c([2H])c([2H])c([2H])c7c6c5c4[2H])c([2H])c3[2H])c3c([2H])c([2H])c([2H])c([2H])c23)c([2H])c1[2H]]

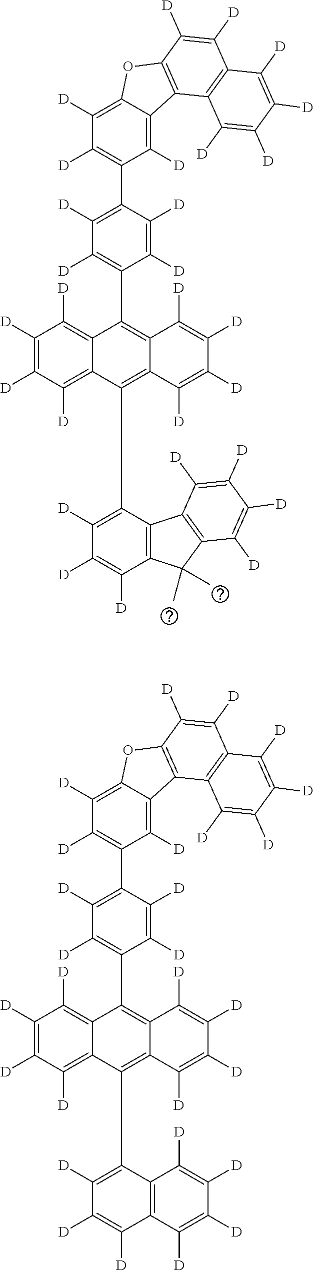 [SMILES: [2H]c1c([2H])c([2H])c2c(c1[2H])-c1c(-c3c4c([2H])c([2H])c([2H])c([2H])c4c(-c4c([2H])c([2H])c(-c5c([2H])c([2H])c6oc7c([2H])c([2H])c8c([2H])c([2H])c([2H])c([2H])c8c7c6c5[2H])c([2H])c4[2H])c4c([2H])c([2H])c([2H])c([2H])c34)c([2H])c([2H])c([2H])c1C2(C)C ; [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c([2H])c1-c1c([2H])c([2H])c2oc3c([2H])c([2H])c4c([2H])c([2H])c([2H])c([2H])c4c3c2c1[2H]]

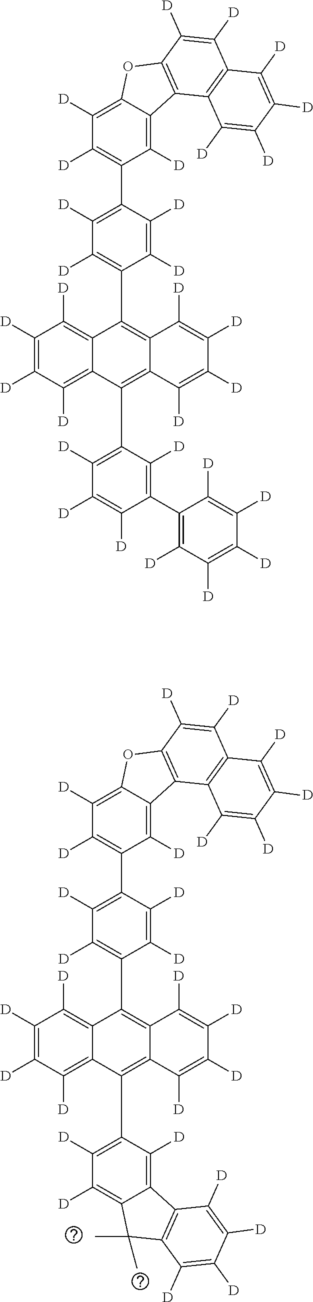

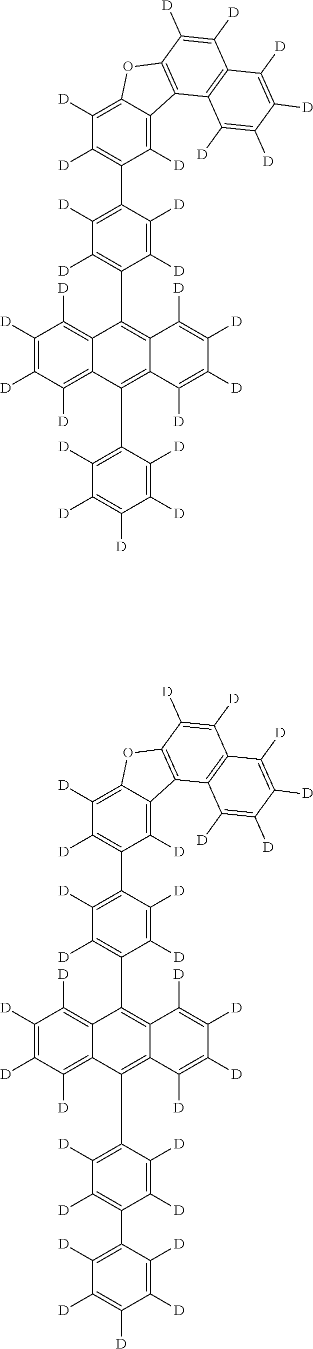 [SMILES: [2H]c1c([2H])c([2H])c(-c2c3c([2H])c([2H])c([2H])c([2H])c3c(-c3c([2H])c([2H])c(-c4c([2H])c([2H])c5oc6c([2H])c([2H])c7c([2H])c([2H])c([2H])c([2H])c7c6c5c4[2H])c([2H])c3[2H])c3c([2H])c([2H])c([2H])c([2H])c23)c([2H])c1[2H] ; [2H]c1c([2H])c([2H])c(-c2c([2H])c([2H])c(-c3c4c([2H])c([2H])c([2H])c([2H])c4c(-c4c([2H])c([2H])c(-c5c([2H])c([2H])c6oc7c([2H])c([2H])c8c([2H])c([2H])c([2H])c([2H])c8c7c6c5[2H])c([2H])c4[2H])c4c([2H])c([2H])c([2H])c([2H])c34)c([2H])c2[2H])c([2H])c1[2H]]

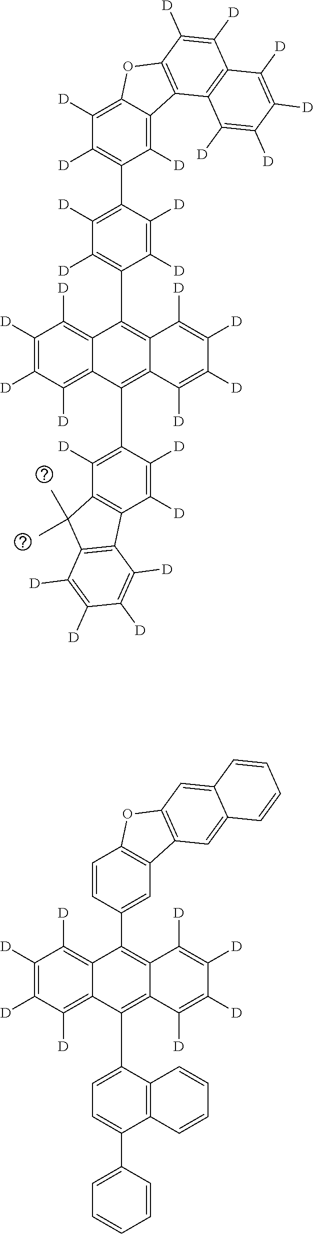 [SMILES: [2H]c1c([2H])c([2H])c2c(c1[2H])-c1c([2H])c([2H])c(-c3c4c([2H])c([2H])c([2H])c([2H])c4c(-c4c([2H])c([2H])c(-c5c([2H])c([2H])c6oc7c([2H])c([2H])c8c([2H])c([2H])c([2H])c([2H])c8c7c6c5[2H])c([2H])c4[2H])c4c([2H])c([2H])c([2H])c([2H])c34)c([2H])c1C2(C)C ; [2H]c1c([2H])c([2H])c2c(-c3ccc(-c4ccccc4)c4ccccc34)c3c([2H])c([2H])c([2H])c([2H])c3c(-c3ccc4oc5cc6ccccc6cc5c4c3)c2c1[2H]]

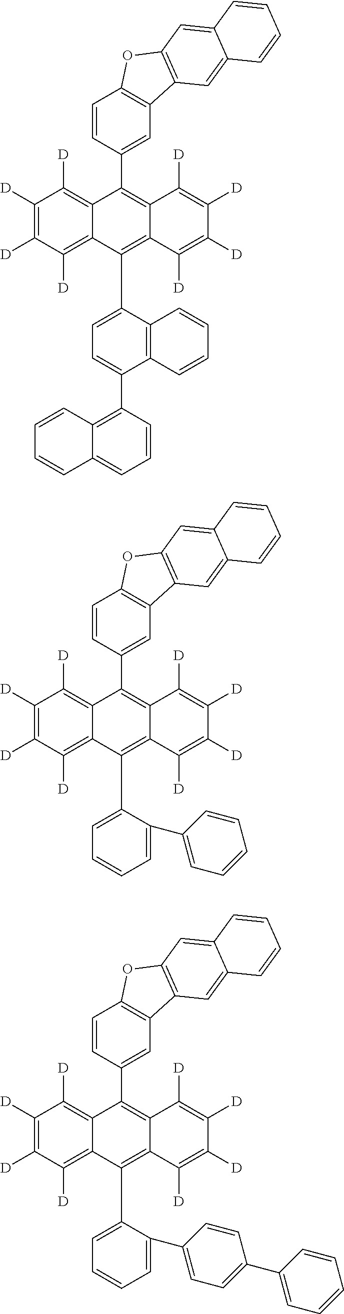

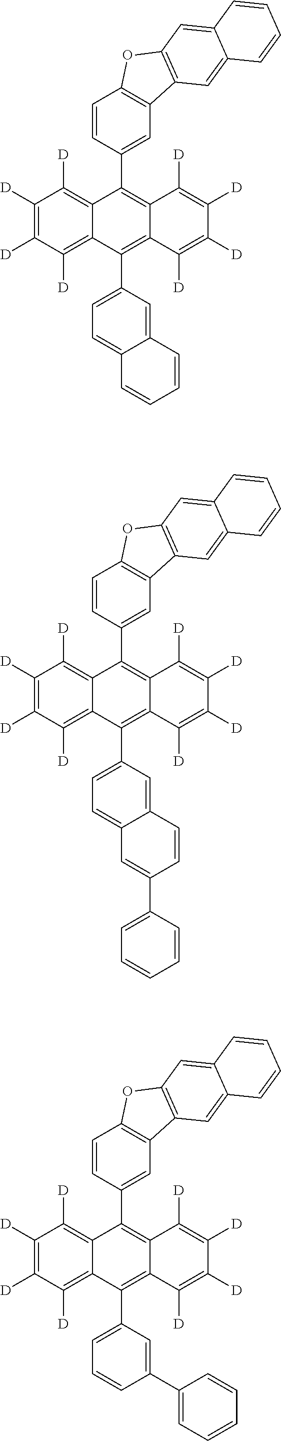 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4cc(-c5ccccc5)ccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccccc4)c3)c2c1[2H]]

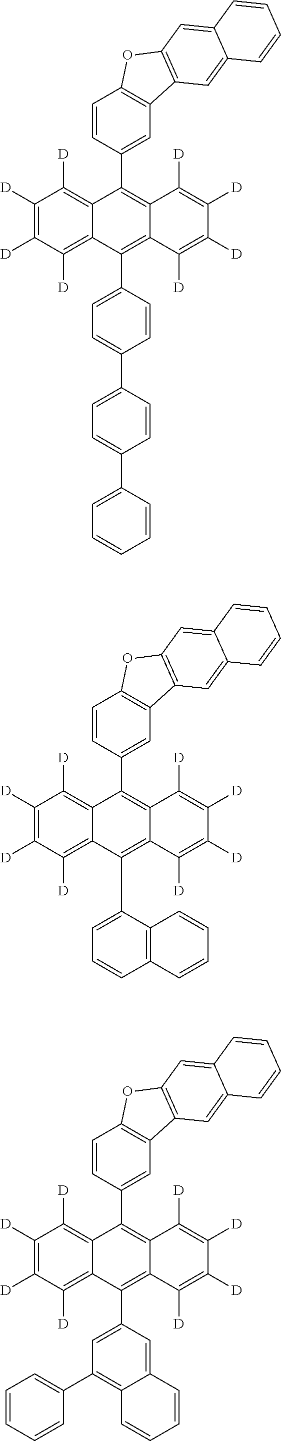 [SMILES: [2H]c1c([2H])c([2H])c2c(-c3ccc4oc5cc6ccccc6cc5c4c3)c3c([2H])c([2H])c([2H])c([2H])c3c(-c3ccc(-c4ccc(-c5ccccc5)cc4)cc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cc(-c4ccccc4)c4ccccc4c3)c2c1[2H]]

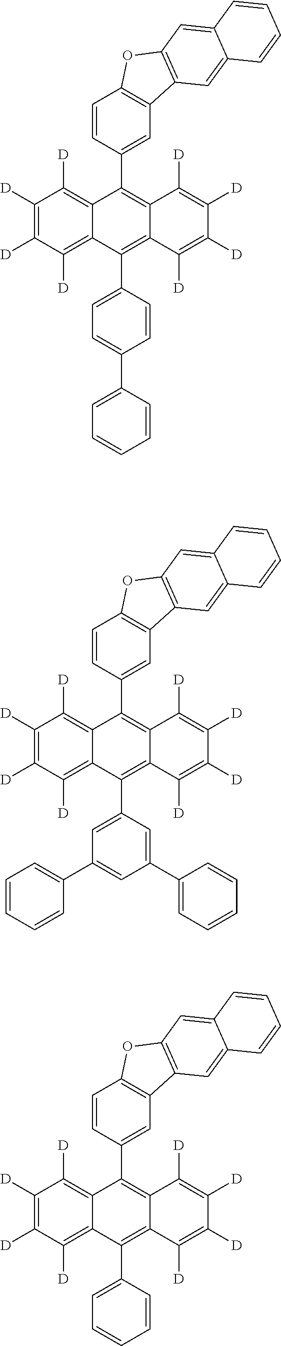 [SMILES: [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H]]

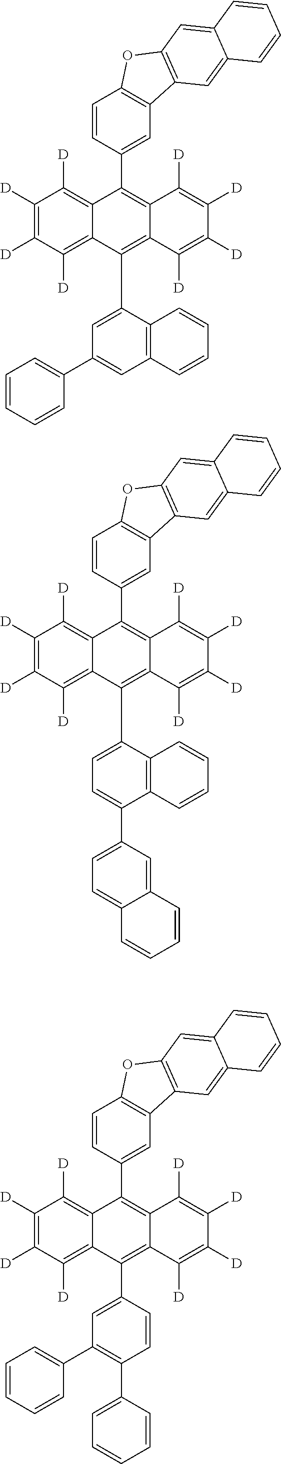 [SMILES: [2H]c1c([2H])c([2H])c2c(-c3cc(-c4ccccc4)cc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc(-c4ccc5ccccc5c4)c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)c(-c4ccccc4)c3)c2c1[2H]]

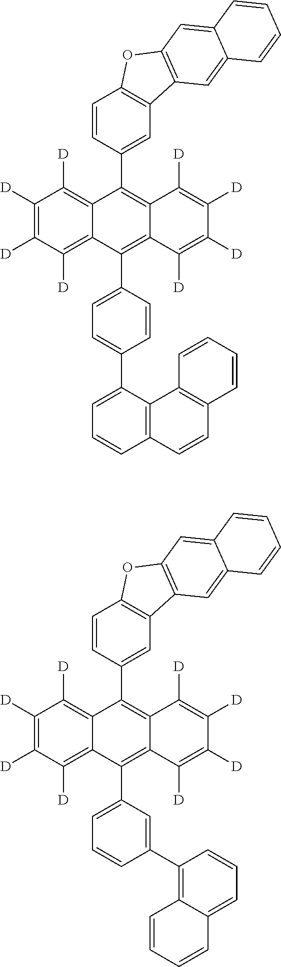 [SMILES: [2H]c1c([2H])c([2H])c2c(-c3ccc4oc5cc6ccccc6cc5c4c3)c3c([2H])c([2H])c([2H])c([2H])c3c(-c3ccc(-c4cccc5ccc6ccccc6c45)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H]]

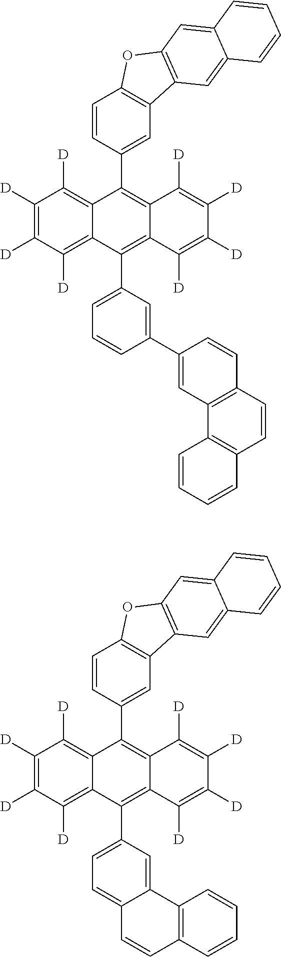 [SMILES: [2H]c1c([2H])c([2H])c2c(-c3ccc4oc5cc6ccccc6cc5c4c3)c3c([2H])c([2H])c([2H])c([2H])c3c(-c3cccc(-c4ccc5ccc6ccccc6c5c4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4ccc5ccccc5c4c3)c2c1[2H]]

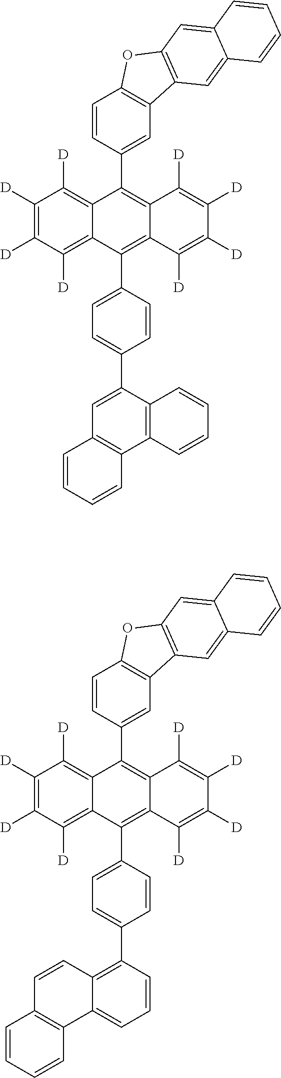 [SMILES: [2H]c1c([2H])c([2H])c2c(-c3ccc4oc5cc6ccccc6cc5c4c3)c3c([2H])c([2H])c([2H])c([2H])c3c(-c3ccc(-c4cc5ccccc5c5ccccc45)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4cccc5c4ccc4ccccc45)cc3)c2c1[2H]]

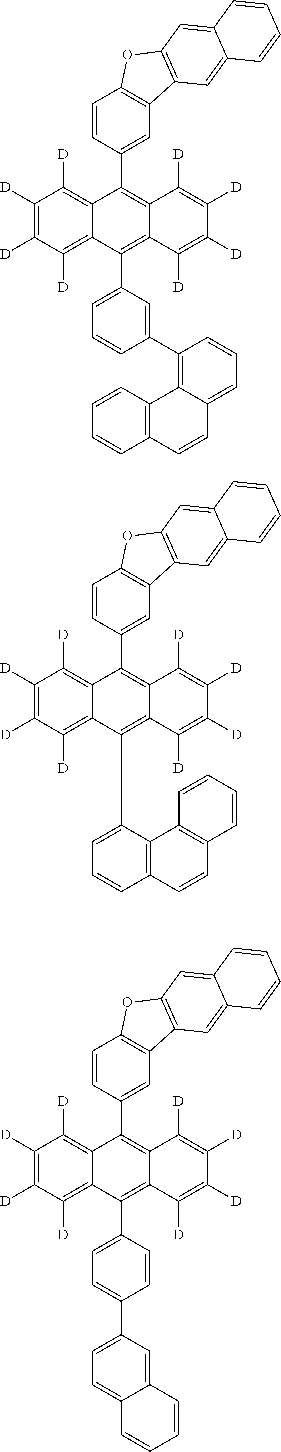 [SMILES: [2H]c1c([2H])c([2H])c2c(-c3ccc4oc5cc6ccccc6cc5c4c3)c3c([2H])c([2H])c([2H])c([2H])c3c(-c3cccc(-c4cccc5ccc6ccccc6c45)c3)c2c1[2H] ; [2H]c1c([2H])c([2H])c2c(-c3cccc4ccc5ccccc5c34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccc5ccccc5c4)cc3)c2c1[2H]]

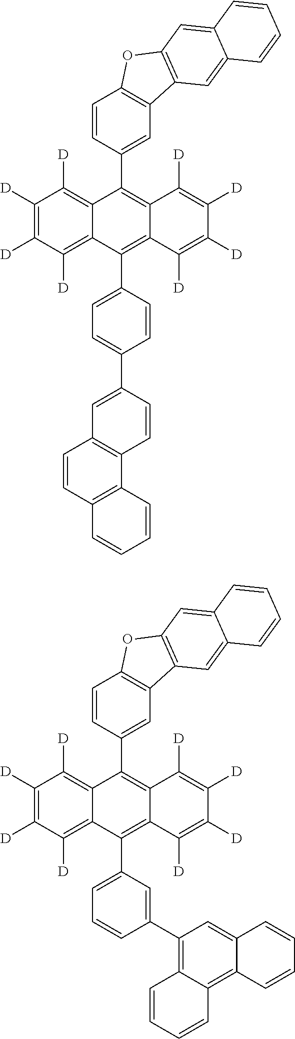 [SMILES: [2H]c1c([2H])c([2H])c2c(-c3ccc4oc5cc6ccccc6cc5c4c3)c3c([2H])c([2H])c([2H])c([2H])c3c(-c3ccc(-c4ccc5c(ccc6ccccc65)c4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cc5ccccc5c5ccccc45)c3)c2c1[2H]]

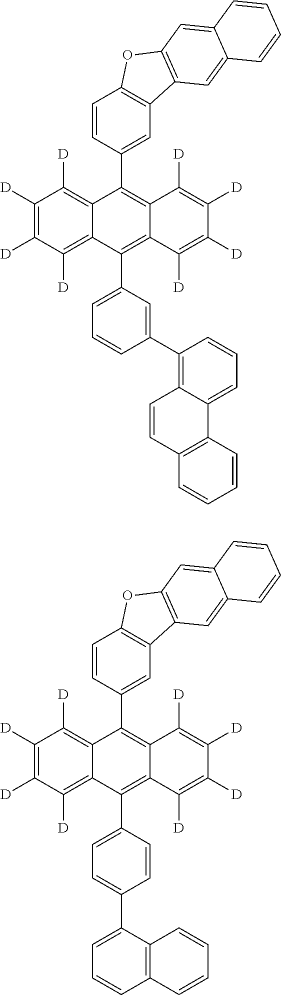

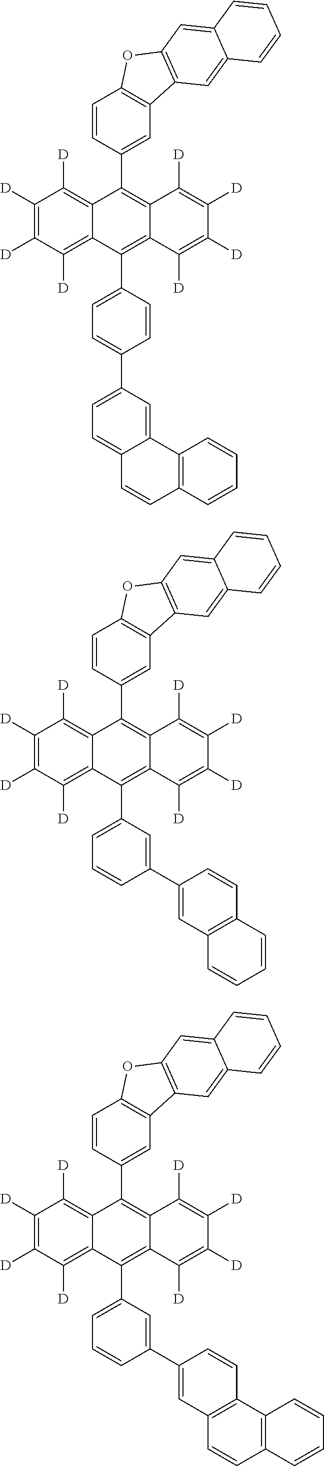

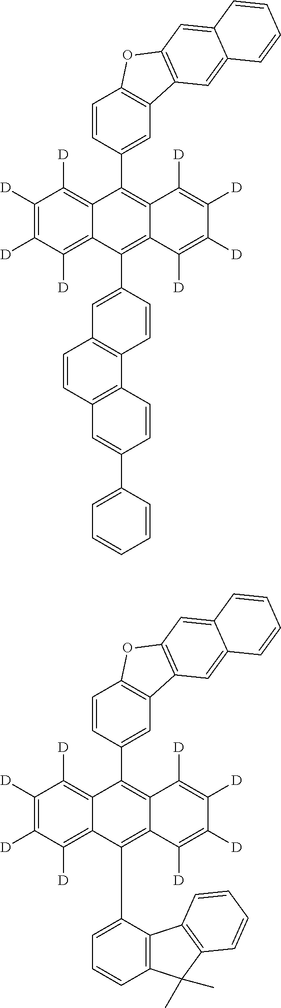 [SMILES: [2H]c1c([2H])c([2H])c2c(-c3ccc4oc5cc6ccccc6cc5c4c3)c3c([2H])c([2H])c([2H])c([2H])c3c(-c3ccc4c(ccc5cc(-c6ccccc6)ccc54)c3)c2c1[2H] ; [2H]c1c([2H])c([2H])c2c(-c3cccc4c3-c3ccccc3C4(C)C)c3c([2H])c([2H])c([2H])c([2H])c3c(-c3ccc4oc5cc6ccccc6cc5c4c3)c2c1[2H]]

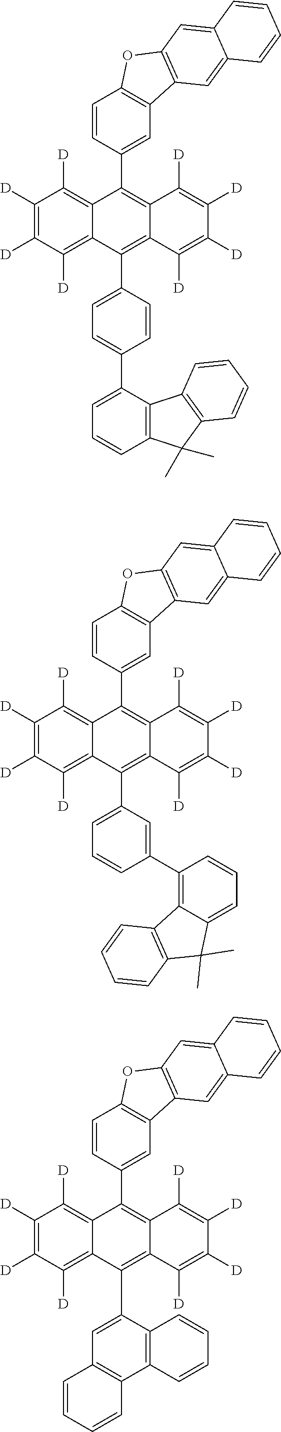

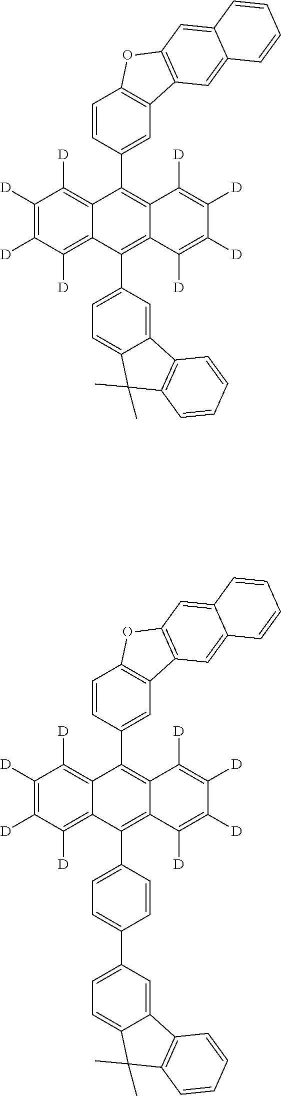 [SMILES: [2H]c1c([2H])c([2H])c2c(-c3ccc4oc5cc6ccccc6cc5c4c3)c3c([2H])c([2H])c([2H])c([2H])c3c(-c3ccc4c(c3)-c3ccccc3C4(C)C)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccc5c(c4)-c4ccccc4C5(C)C)cc3)c2c1[2H]]

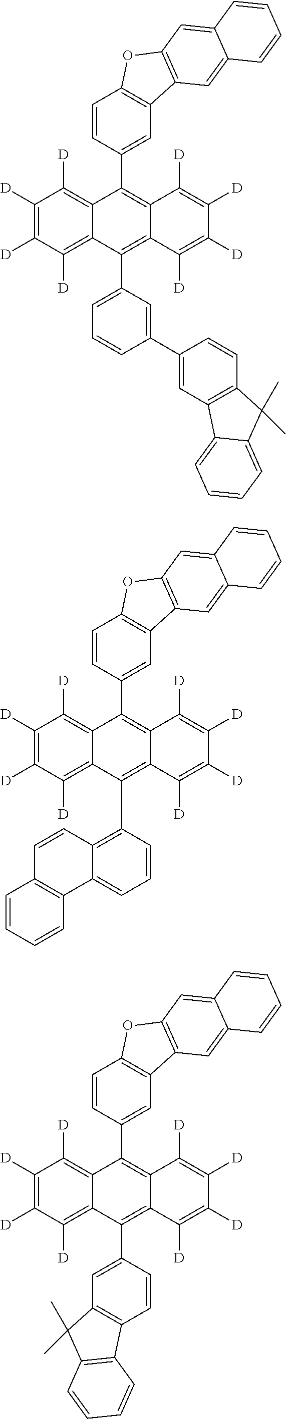

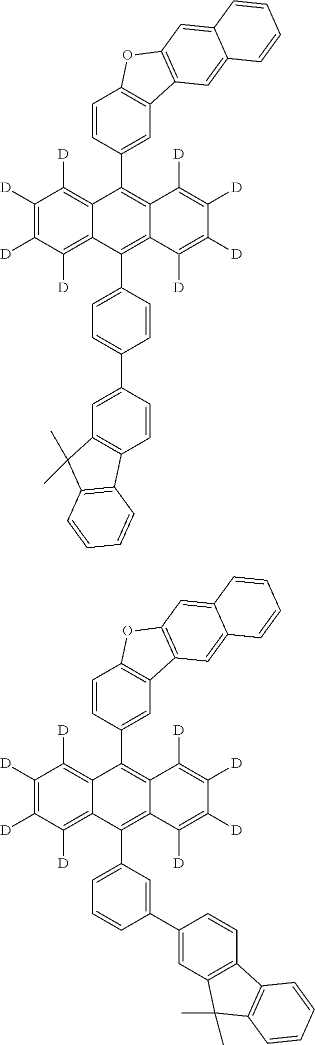 [SMILES: [2H]c1c([2H])c([2H])c2c(-c3ccc4oc5cc6ccccc6cc5c4c3)c3c([2H])c([2H])c([2H])c([2H])c3c(-c3ccc(-c4ccc5c(c4)C(C)(C)c4ccccc4-5)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5c(c4)C(C)(C)c4ccccc4-5)c3)c2c1[2H]]

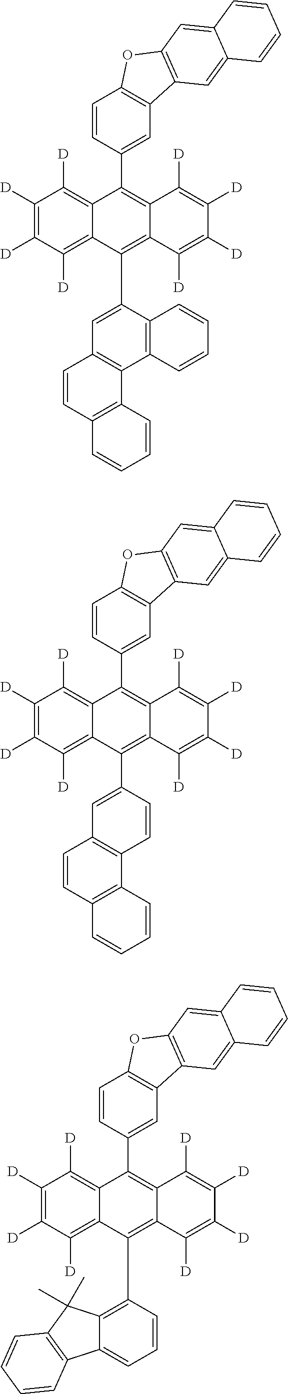 [SMILES: [2H]c1c([2H])c([2H])c2c(-c3cc4ccc5ccccc5c4c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4c(ccc5ccccc54)c3)c2c1[2H] ; [2H]c1c([2H])c([2H])c2c(-c3cccc4c3C(C)(C)c3ccccc3-4)c3c([2H])c([2H])c([2H])c([2H])c3c(-c3ccc4oc5cc6ccccc6cc5c4c3)c2c1[2H]]

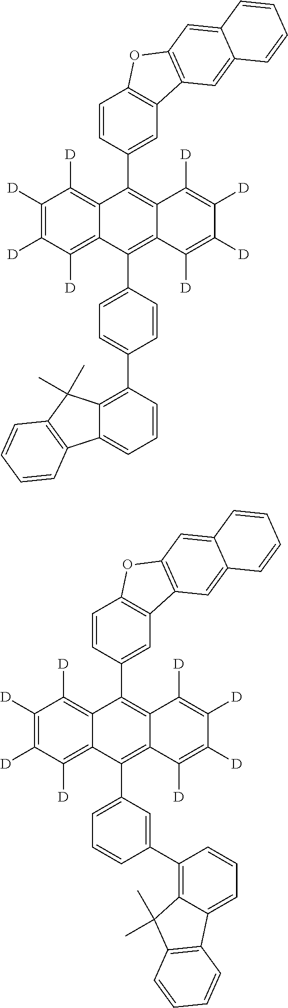 [SMILES: [2H]c1c([2H])c([2H])c2c(-c3ccc4oc5cc6ccccc6cc5c4c3)c3c([2H])c([2H])c([2H])c([2H])c3c(-c3ccc(-c4cccc5c4C(C)(C)c4ccccc4-5)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5c4C(C)(C)c4ccccc4-5)c3)c2c1[2H]]

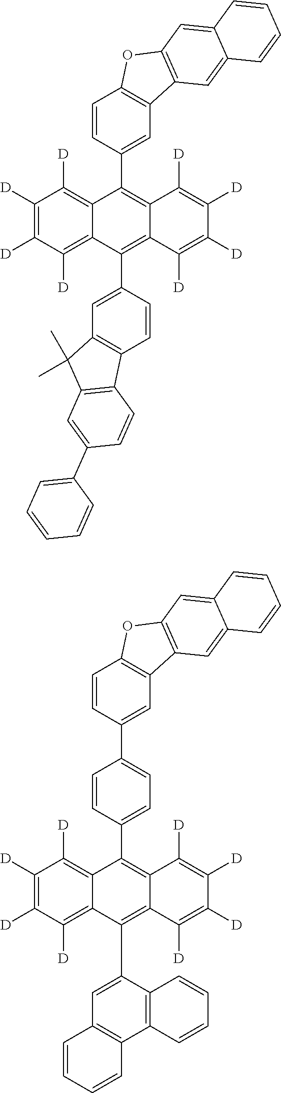 [SMILES: [2H]c1c([2H])c([2H])c2c(-c3ccc4oc5cc6ccccc6cc5c4c3)c3c([2H])c([2H])c([2H])c([2H])c3c(-c3ccc4c(c3)C(C)(C)c3cc(-c5ccccc5)ccc3-4)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5oc6cc7ccccc7cc6c5c4)cc3)c2c1[2H]]

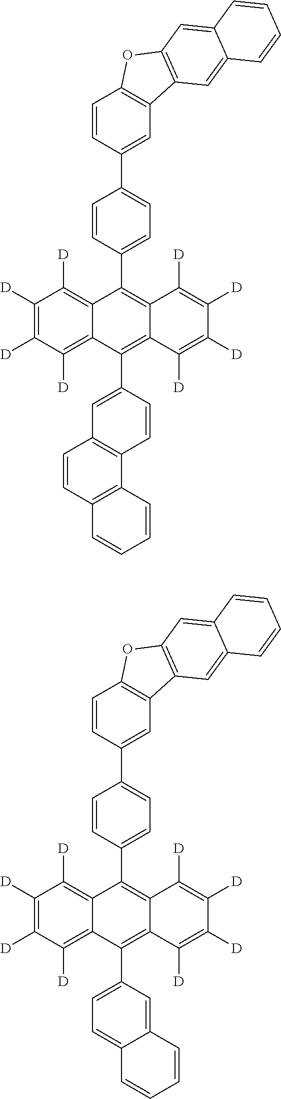 [SMILES: [2H]c1c([2H])c([2H])c2c(-c3ccc4c(ccc5ccccc54)c3)c3c([2H])c([2H])c([2H])c([2H])c3c(-c3ccc(-c4ccc5oc6cc7ccccc7cc6c5c4)cc3)c2c1[2H] ; [2H]c1c([2H])c([2H])c2c(-c3ccc4ccccc4c3)c3c([2H])c([2H])c([2H])c([2H])c3c(-c3ccc(-c4ccc5oc6cc7ccccc7cc6c5c4)cc3)c2c1[2H]]

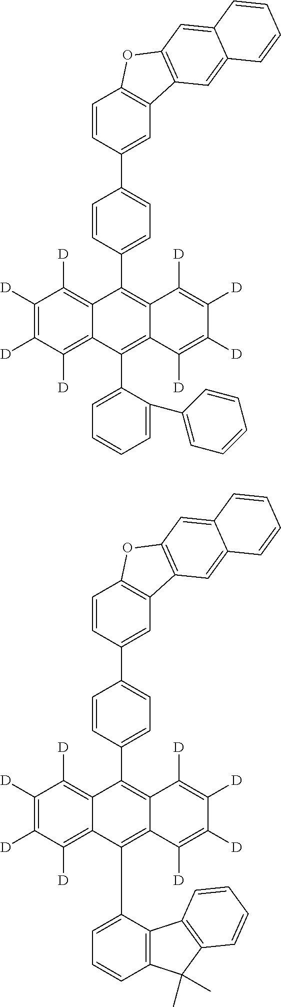

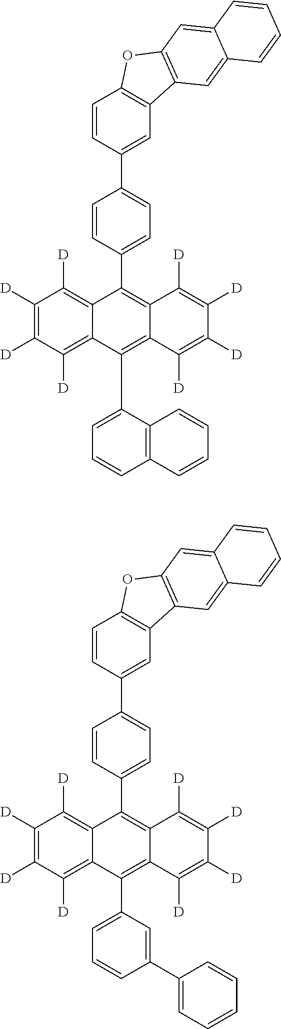 [SMILES: [2H]c1c([2H])c([2H])c2c(-c3cccc4ccccc34)c3c([2H])c([2H])c([2H])c([2H])c3c(-c3ccc(-c4ccc5oc6cc7ccccc7cc6c5c4)cc3)c2c1[2H] ; [2H]c1c([2H])c([2H])c2c(-c3cccc(-c4ccccc4)c3)c3c([2H])c([2H])c([2H])c([2H])c3c(-c3ccc(-c4ccc5oc6cc7ccccc7cc6c5c4)cc3)c2c1[2H]]

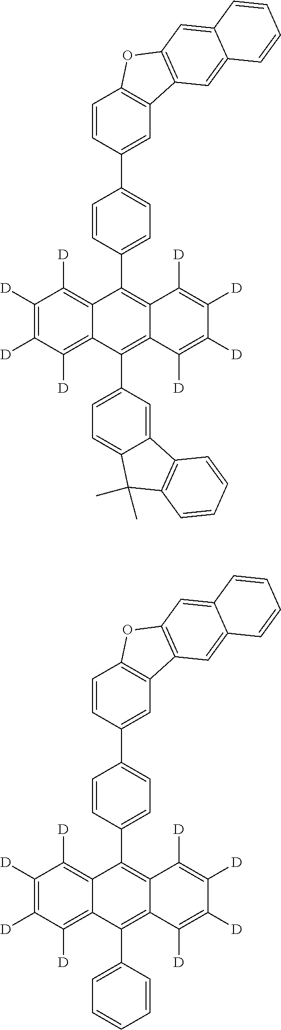 [SMILES: [2H]c1c([2H])c([2H])c2c(-c3ccc4c(c3)-c3ccccc3C4(C)C)c3c([2H])c([2H])c([2H])c([2H])c3c(-c3ccc(-c4ccc5oc6cc7ccccc7cc6c5c4)cc3)c2c1[2H] ; [2H]c1c([2H])c([2H])c2c(-c3ccc(-c4ccc5oc6cc7ccccc7cc6c5c4)cc3)c3c([2H])c([2H])c([2H])c([2H])c3c(-c3ccccc3)c2c1[2H]]

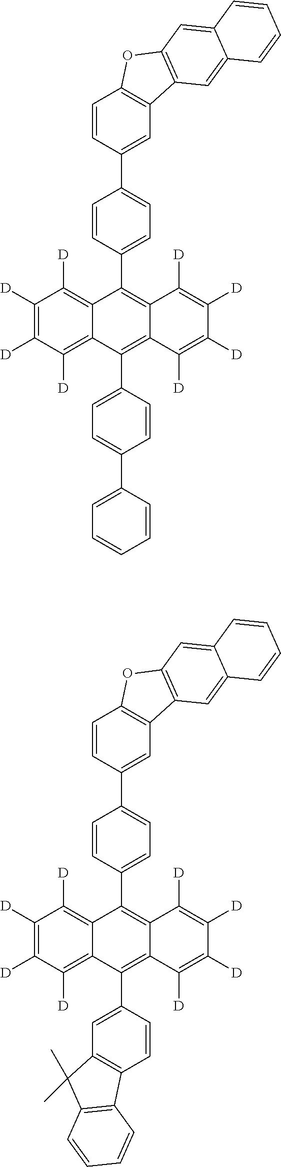 [SMILES: [2H]c1c([2H])c([2H])c2c(-c3ccc(-c4ccc5oc6cc7ccccc7cc6c5c4)cc3)c3c([2H])c([2H])c([2H])c([2H])c3c(-c3ccc(-c4ccccc4)cc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccc5oc6cc7ccccc7cc6c5c4)cc3)c2c1[2H]]

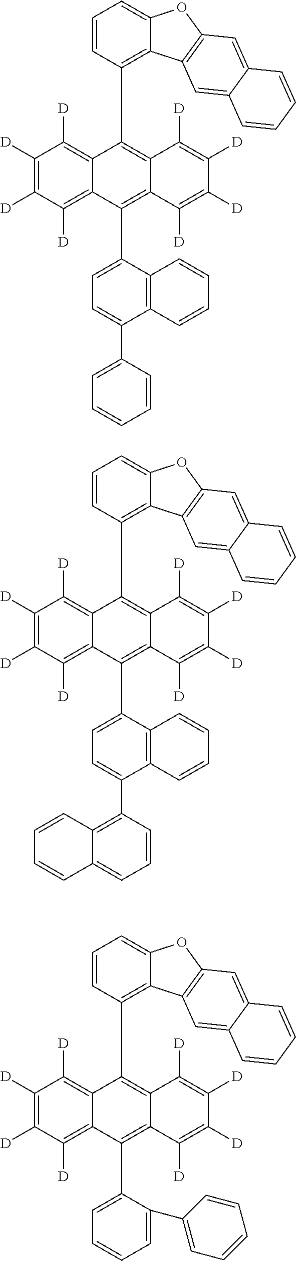

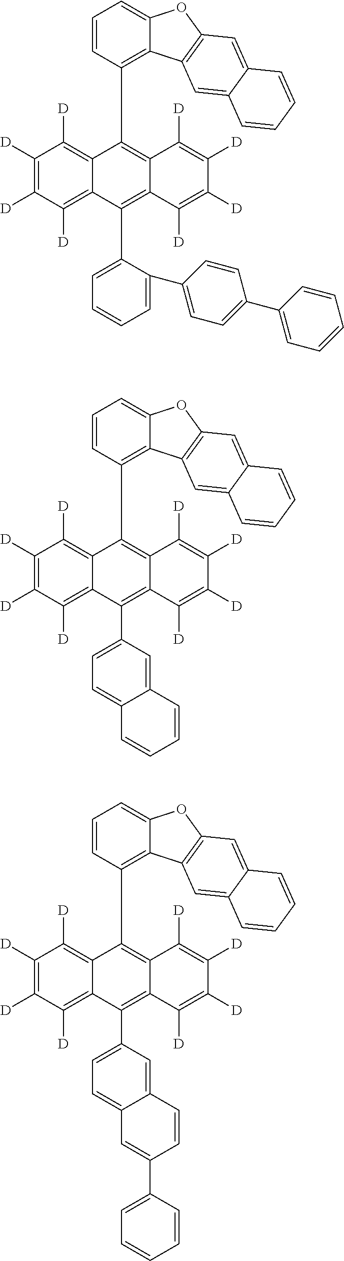 [SMILES: [2H]c1c([2H])c([2H])c2c(-c3cccc4oc5cc6ccccc6cc5c34)c3c([2H])c([2H])c([2H])c([2H])c3c(-c3ccccc3-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3ccc4ccccc4c3)c2c1[2H] ; [2H]c1c([2H])c([2H])c2c(-c3cccc4oc5cc6ccccc6cc5c34)c3c([2H])c([2H])c([2H])c([2H])c3c(-c3ccc4cc(-c5ccccc5)ccc4c3)c2c1[2H]]

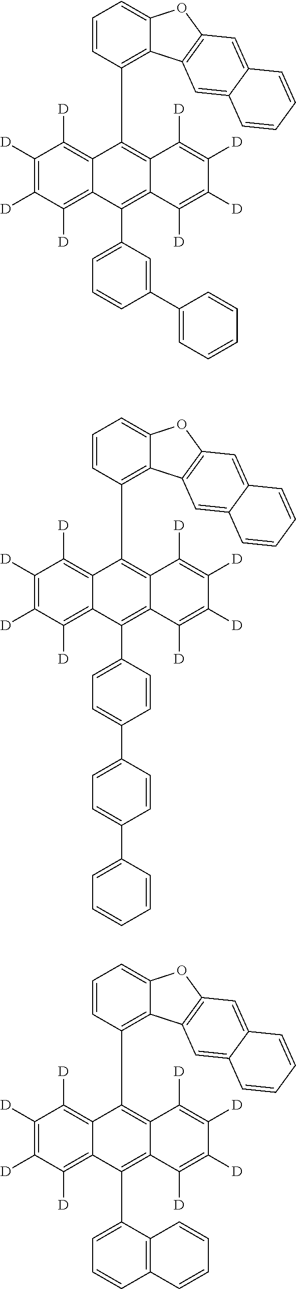

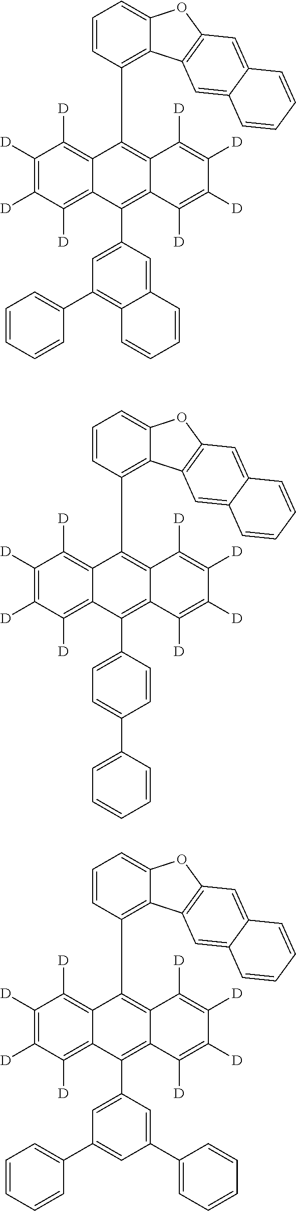

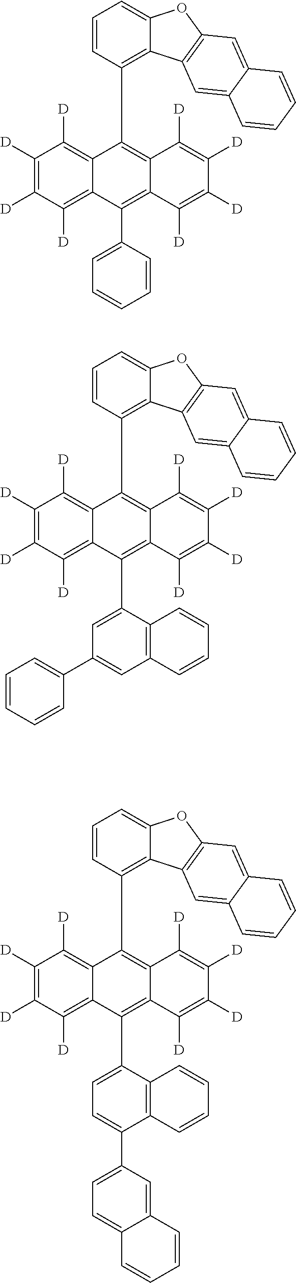

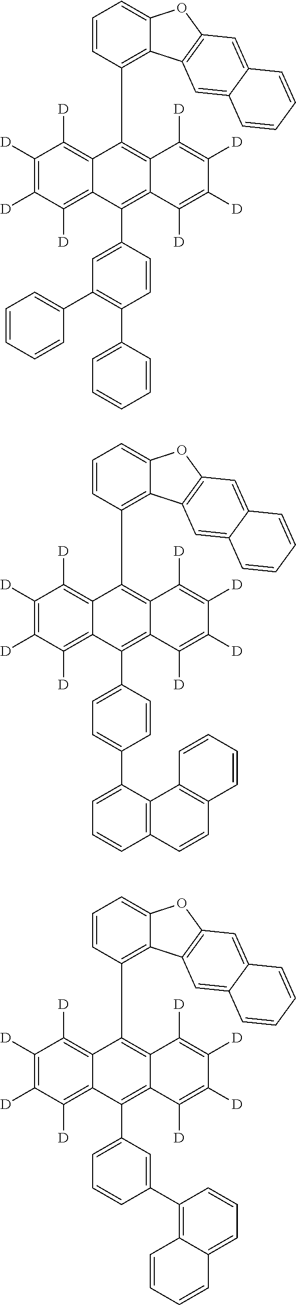

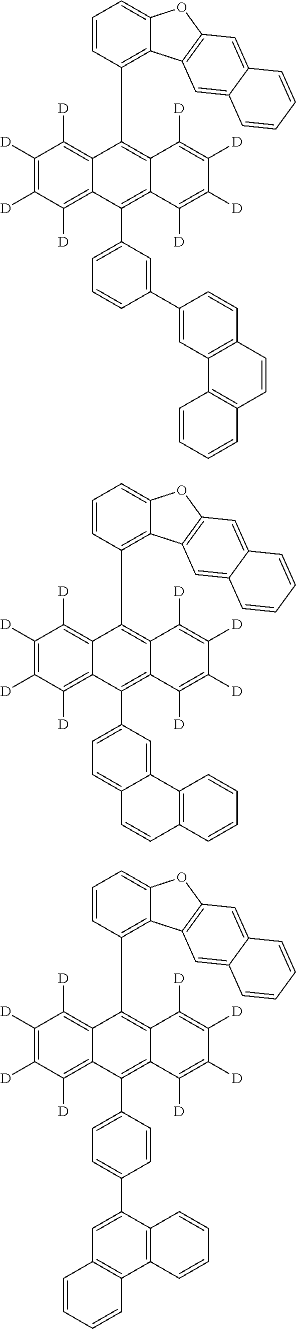

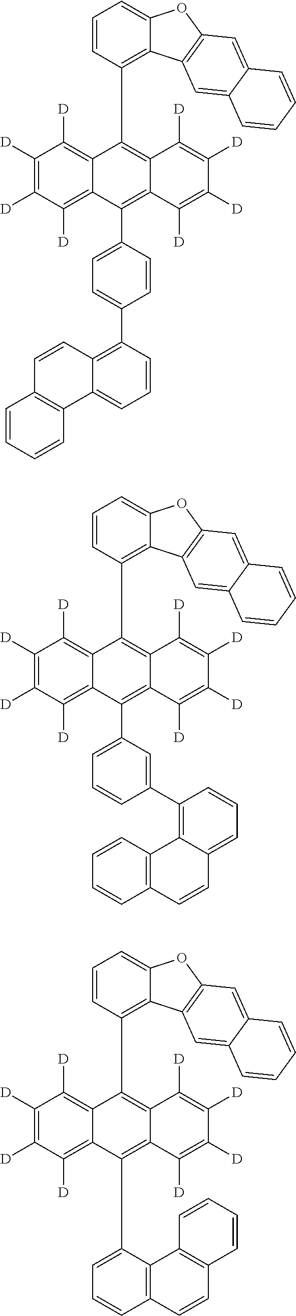 [SMILES: [2H]c1c([2H])c([2H])c2c(-c3cccc4oc5cc6ccccc6cc5c34)c3c([2H])c([2H])c([2H])c([2H])c3c(-c3ccc(-c4cccc5c4ccc4ccccc45)cc3)c2c1[2H] ; [2H]c1c([2H])c([2H])c2c(-c3cccc4oc5cc6ccccc6cc5c34)c3c([2H])c([2H])c([2H])c([2H])c3c(-c3cccc(-c4cccc5ccc6ccccc6c45)c3)c2c1[2H] ; [2H]c1c([2H])c([2H])c2c(-c3cccc4oc5cc6ccccc6cc5c34)c3c([2H])c([2H])c([2H])c([2H])c3c(-c3cccc4ccc5ccccc5c34)c2c1[2H]]

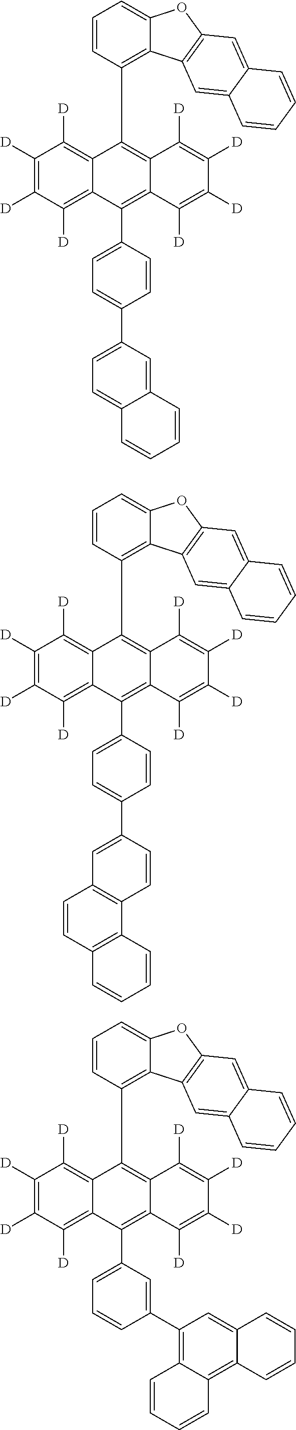

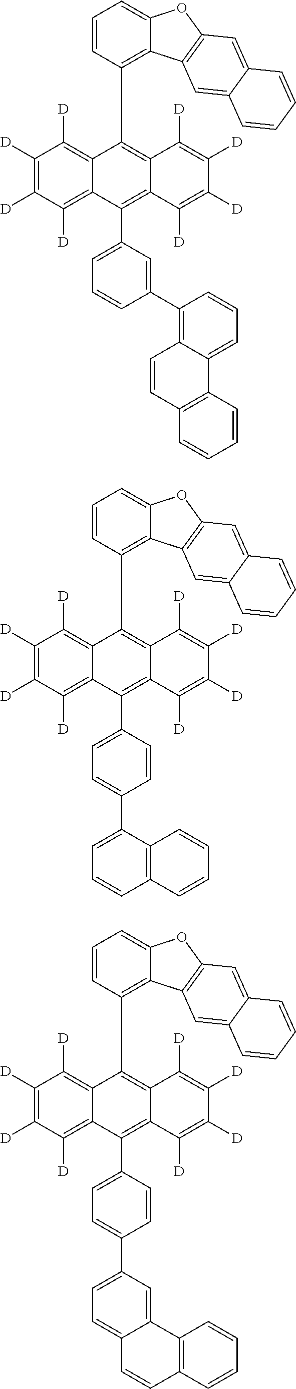

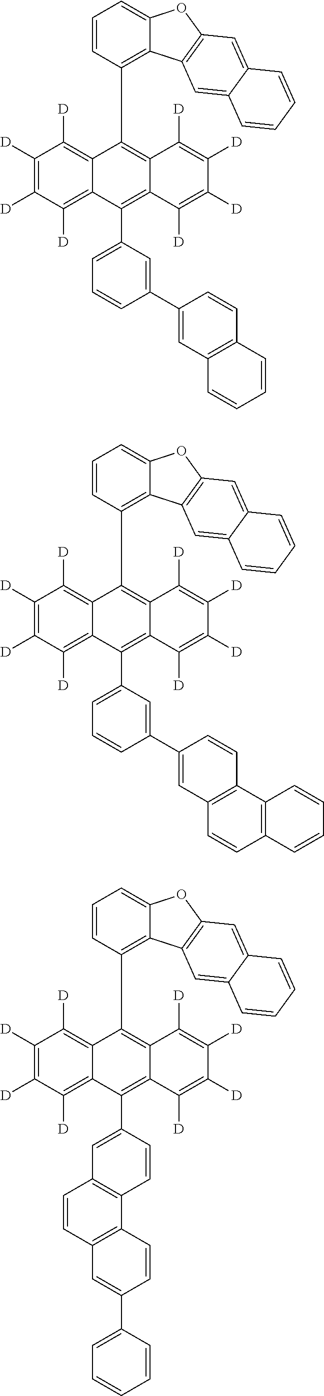 [SMILES: [2H]c1c([2H])c([2H])c2c(-c3cccc4oc5cc6ccccc6cc5c34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4oc5cc6ccccc6cc5c34)c3c([2H])c([2H])c([2H])c([2H])c3c(-c3cccc(-c4ccc5c(ccc6ccccc65)c4)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc4c(ccc5cc(-c6ccccc6)ccc54)c3)c2c1[2H]]

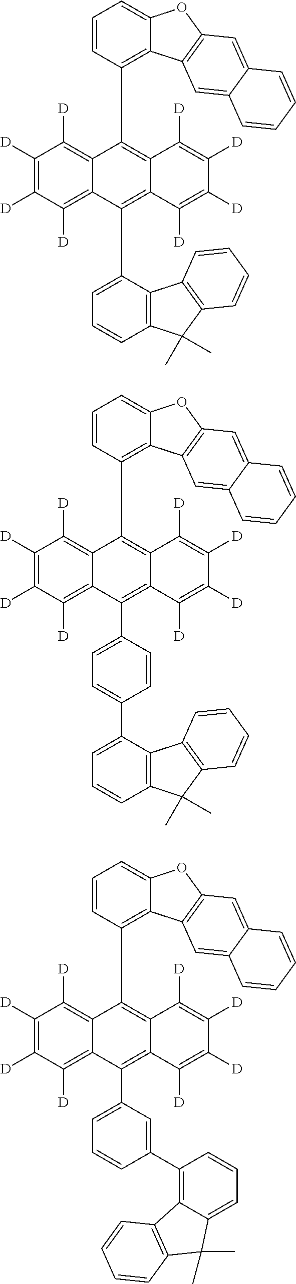

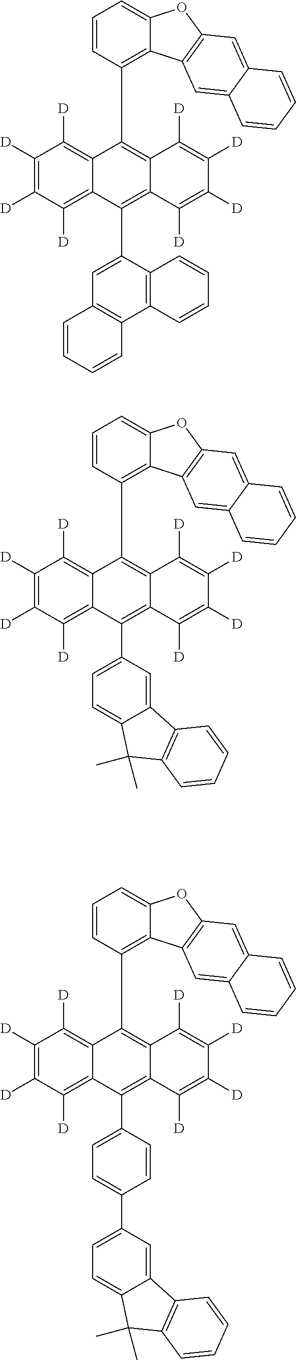 [SMILES: [2H]c1c([2H])c([2H])c2c(-c3cccc4oc5cc6ccccc6cc5c34)c3c([2H])c([2H])c([2H])c([2H])c3c(-c3cc4ccccc4c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc4c(c3)-c3ccccc3C4(C)C)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccc5c(c4)-c4ccccc4C5(C)C)cc3)c2c1[2H]]

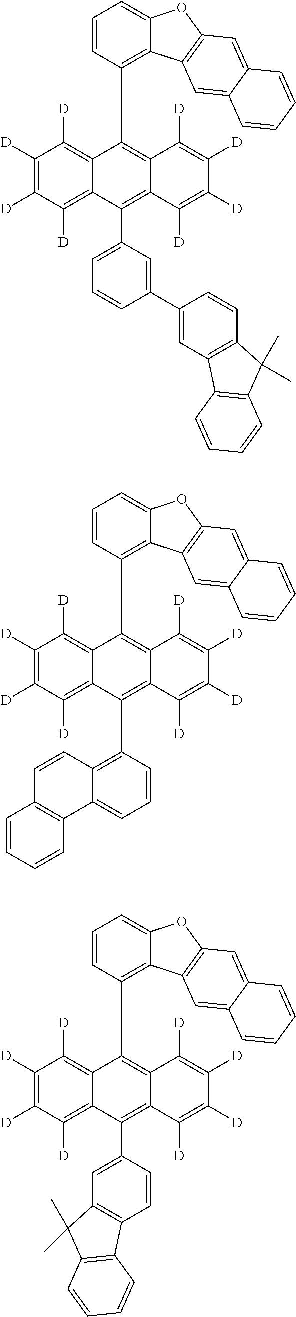 [SMILES: [2H]c1c([2H])c([2H])c2c(-c3cccc4oc5cc6ccccc6cc5c34)c3c([2H])c([2H])c([2H])c([2H])c3c(-c3cccc(-c4ccc5c(c4)-c4ccccc4C5(C)C)c3)c2c1[2H] ; [2H]c1c([2H])c([2H])c2c(-c3cccc4oc5cc6ccccc6cc5c34)c3c([2H])c([2H])c([2H])c([2H])c3c(-c3cccc4c3ccc3ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc4c(c3)C(C)(C)c3ccccc3-4)c2c1[2H]]

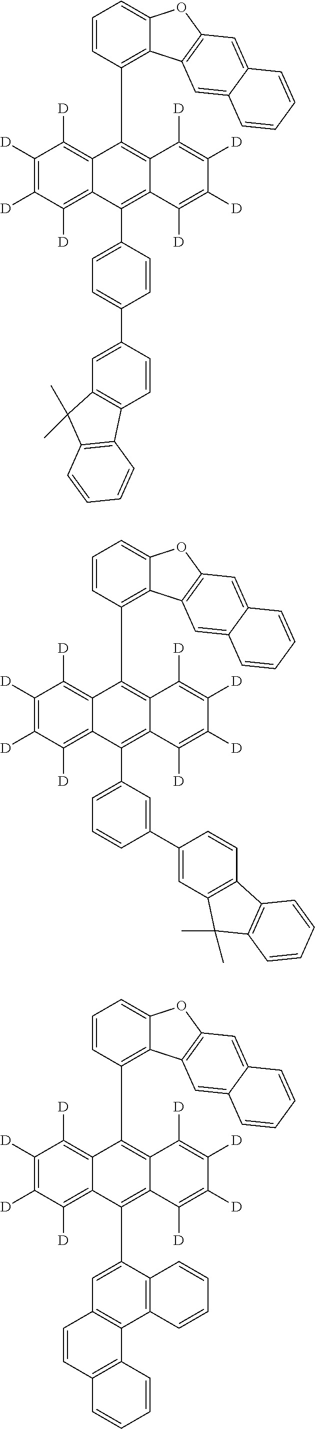

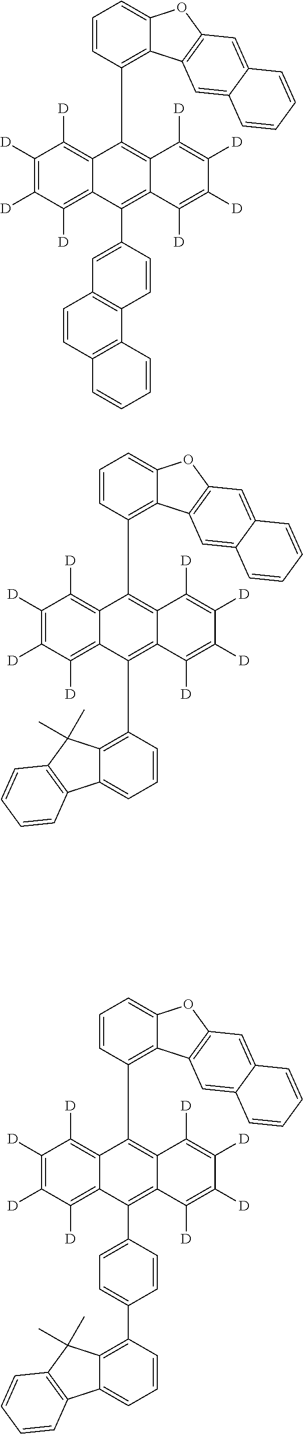

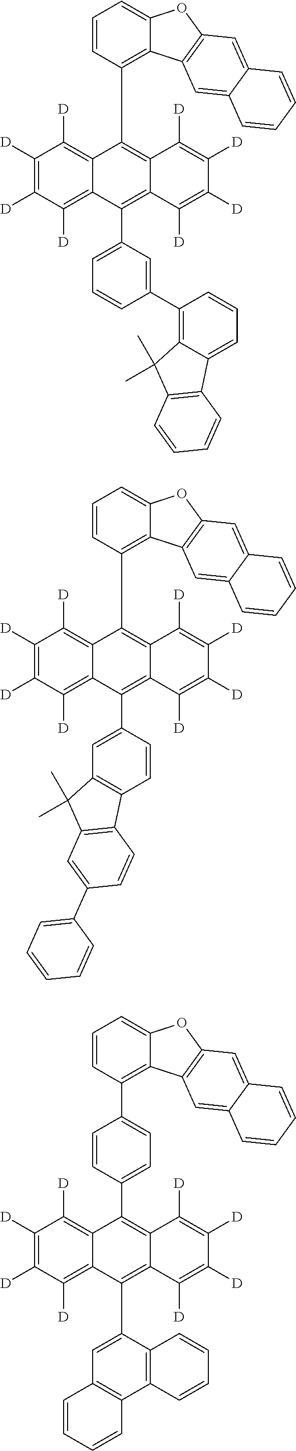

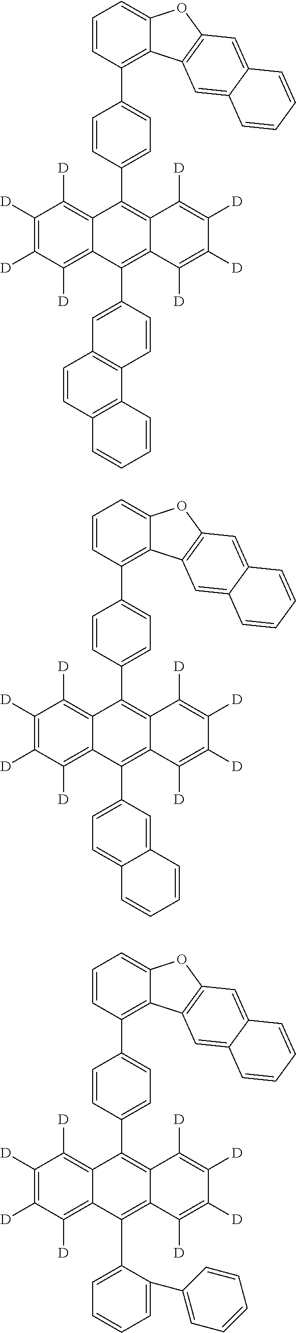

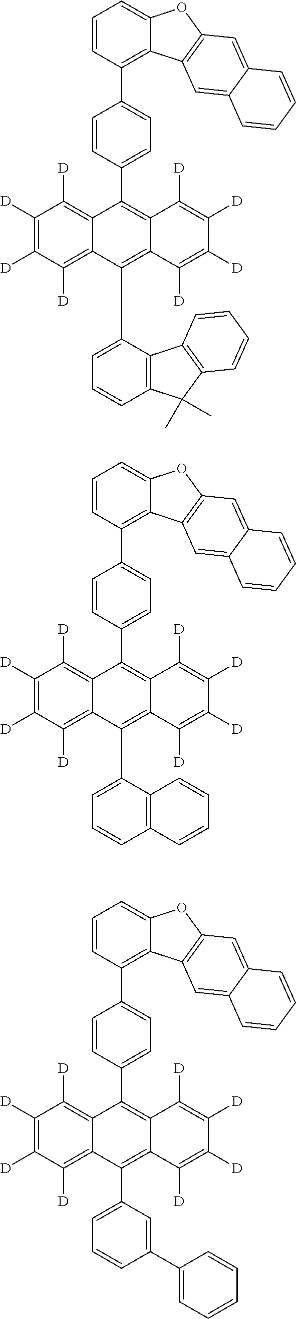

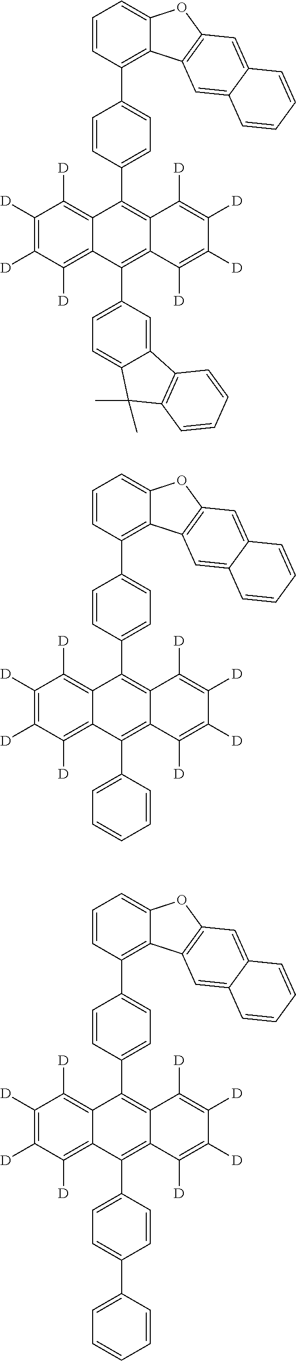

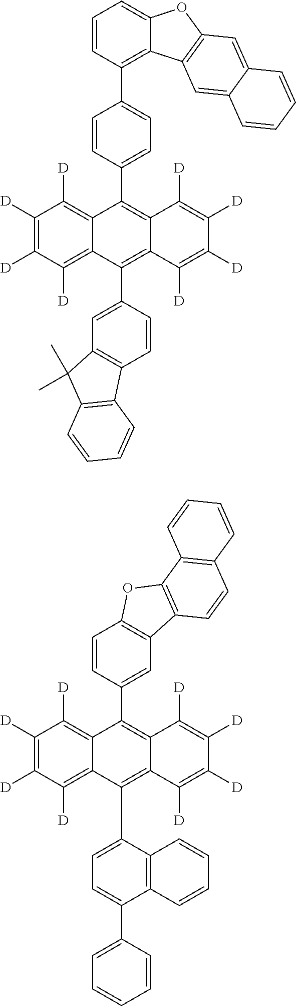 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4cccc5oc6cc7ccccc7cc6c45)cc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4oc5c6ccccc6ccc5c4c3)c2c1[2H]]

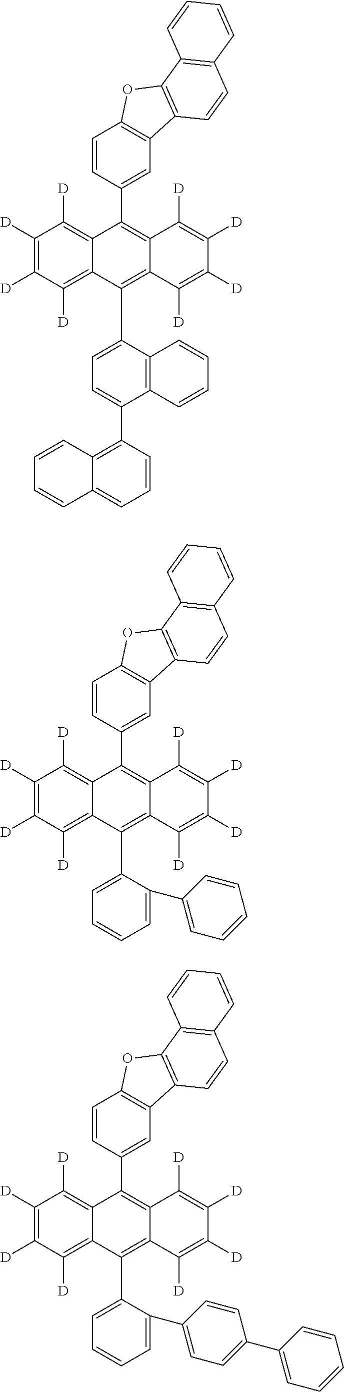

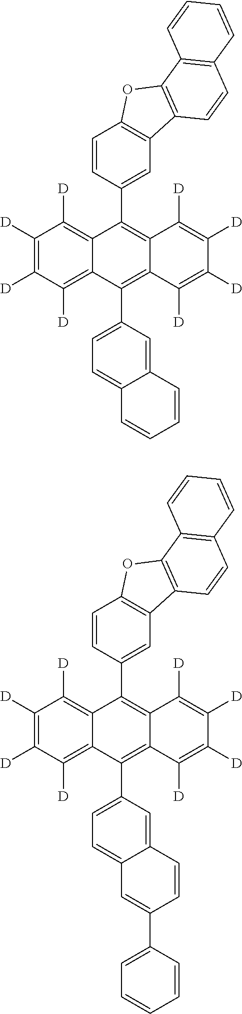 [SMILES: [2H]c1c([2H])c([2H])c2c(-c3ccc4oc5c6ccccc6ccc5c4c3)c3c([2H])c([2H])c([2H])c([2H])c3c(-c3ccc4ccccc4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc4cc(-c5ccccc5)ccc4c3)c2c1[2H]]

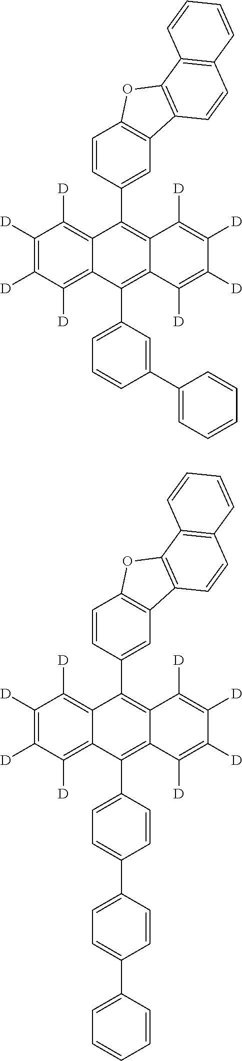

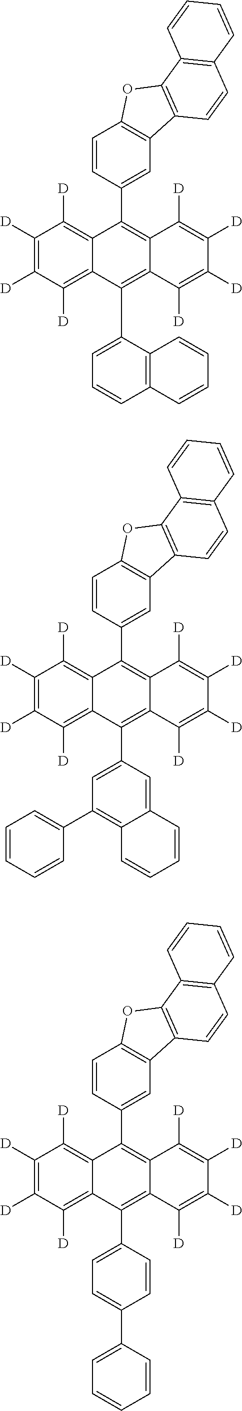

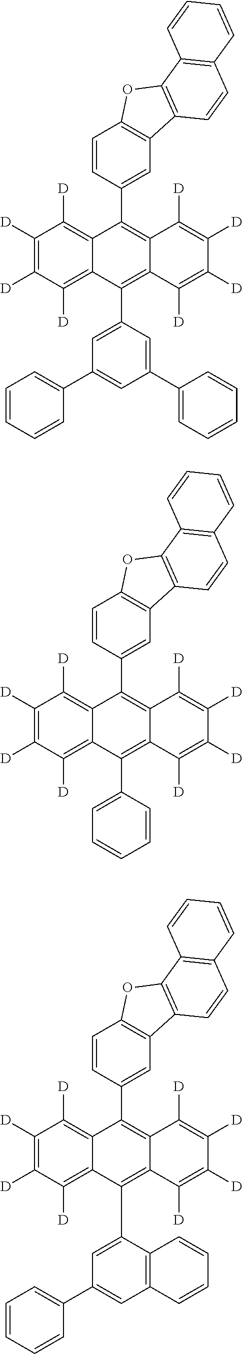

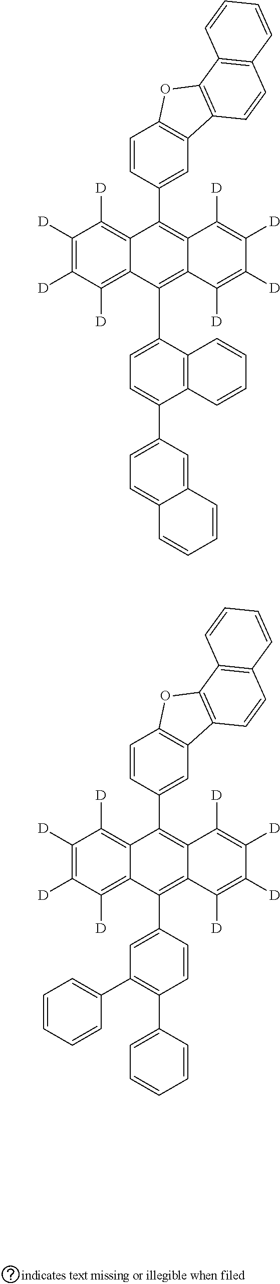 [SMILES: [2H]c1c([2H])c([2H])c2c(-c3ccc(-c4ccc5ccccc5c4)c4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)c(-c4ccccc4)c3)c2c1[2H]]

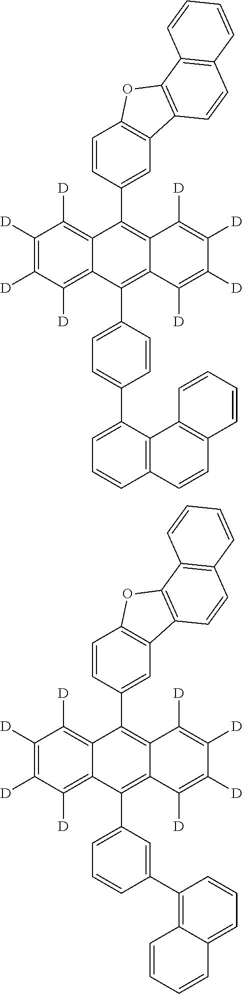

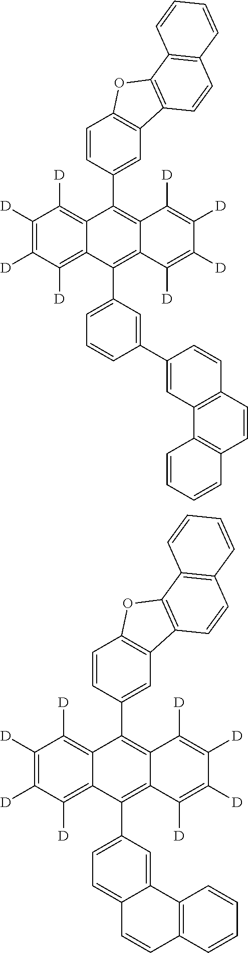

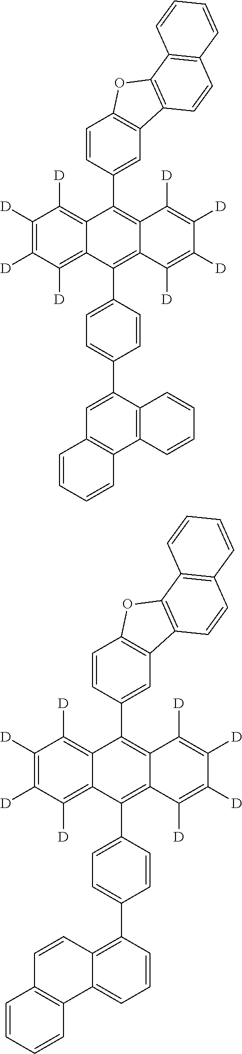 [SMILES: [2H]c1c([2H])c([2H])c2c(-c3ccc4oc5c6ccccc6ccc5c4c3)c3c([2H])c([2H])c([2H])c([2H])c3c(-c3ccc(-c4cc5ccccc5c5ccccc45)cc3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4cccc5c4ccc4ccccc45)cc3)c2c1[2H]]

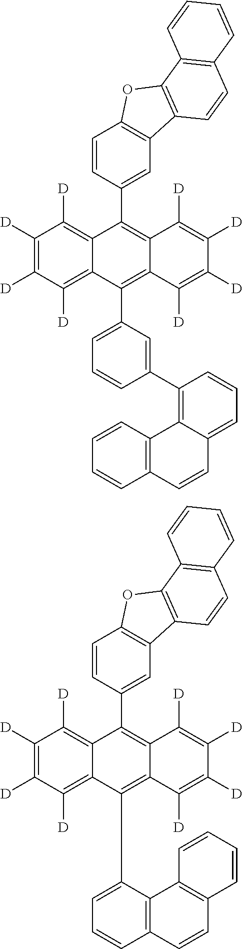

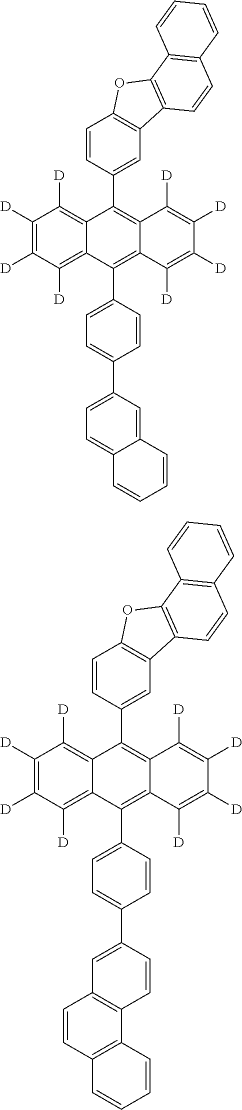

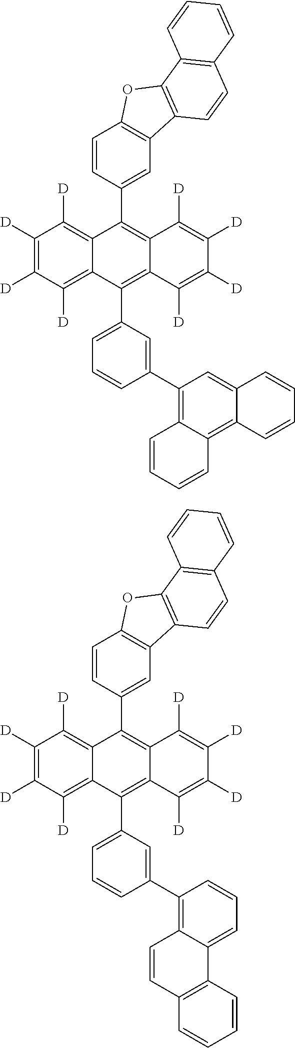

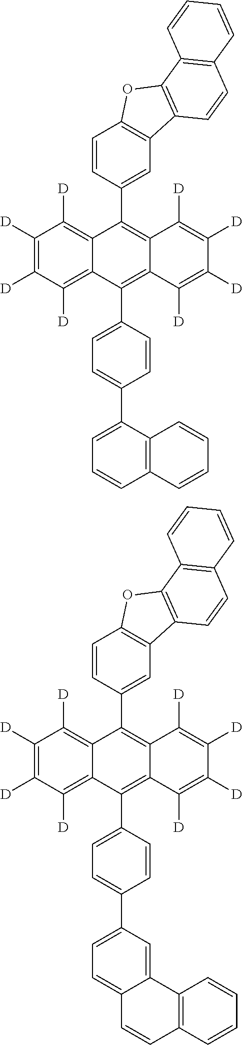

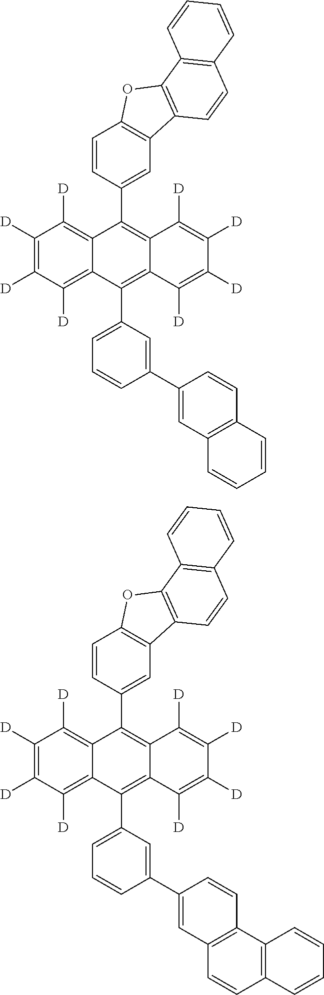

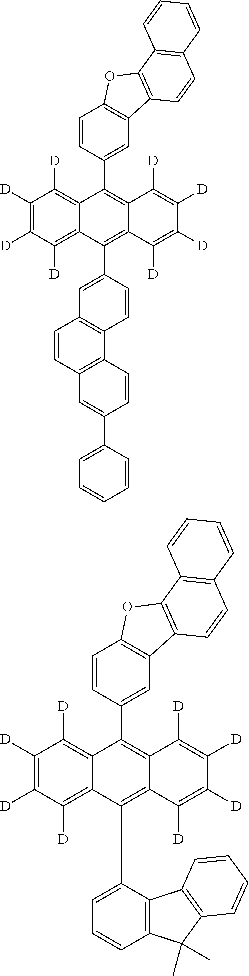 [SMILES: [2H]c1c([2H])c([2H])c2c(-c3ccc4oc5c6ccccc6ccc5c4c3)c3c([2H])c([2H])c([2H])c([2H])c3c(-c3ccc4c(ccc5cc(-c6ccccc6)ccc54)c3)c2c1[2H] ; [2H]c1c([2H])c([2H])c2c(-c3cccc4c3-c3ccccc3C4(C)C)c3c([2H])c([2H])c([2H])c([2H])c3c(-c3ccc4oc5c6ccccc6ccc5c4c3)c2c1[2H]]

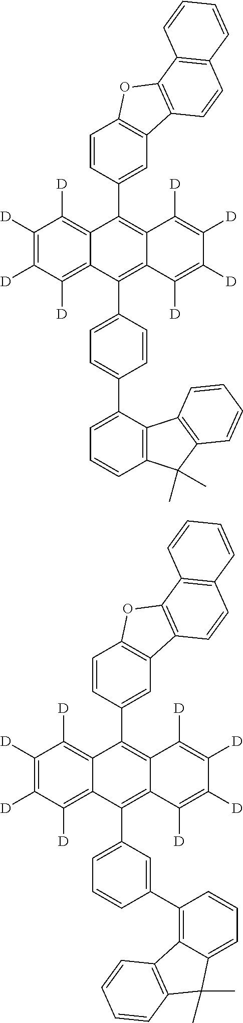

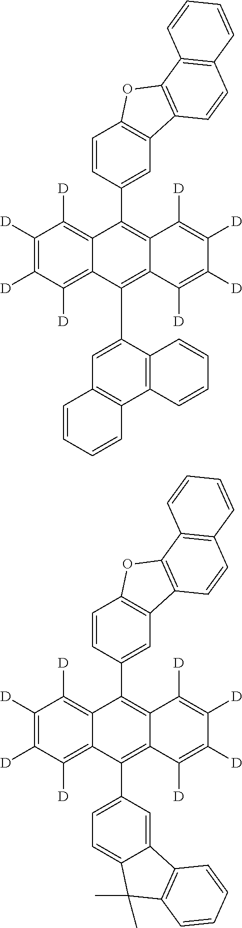 [SMILES: [2H]c1c([2H])c([2H])c2c(-c3cc4ccccc4c4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc4c(c3)-c3ccccc3C4(C)C)c2c1[2H]]

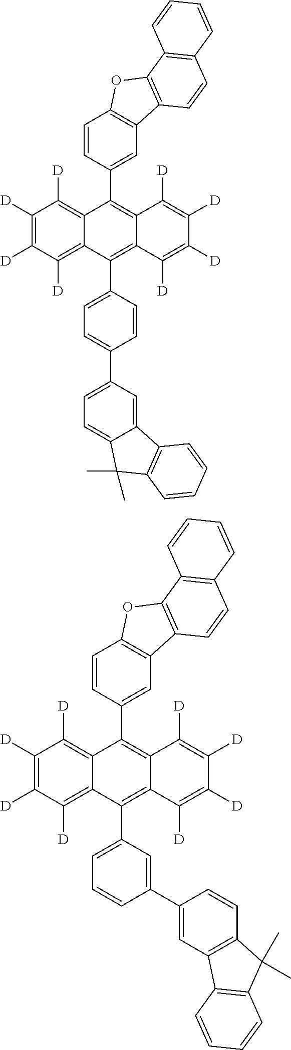

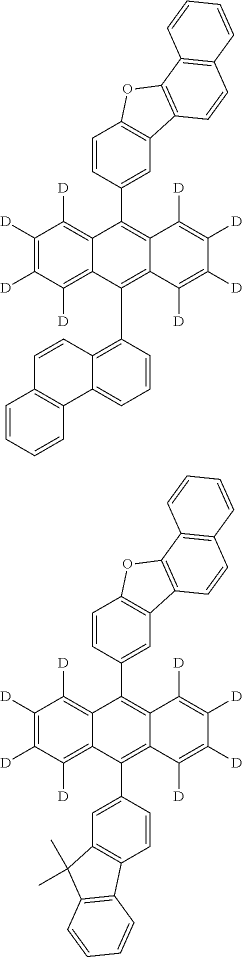 [SMILES: [2H]c1c([2H])c([2H])c2c(-c3cccc4c3ccc3ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc4c(c3)C(C)(C)c3ccccc3-4)c2c1[2H]]

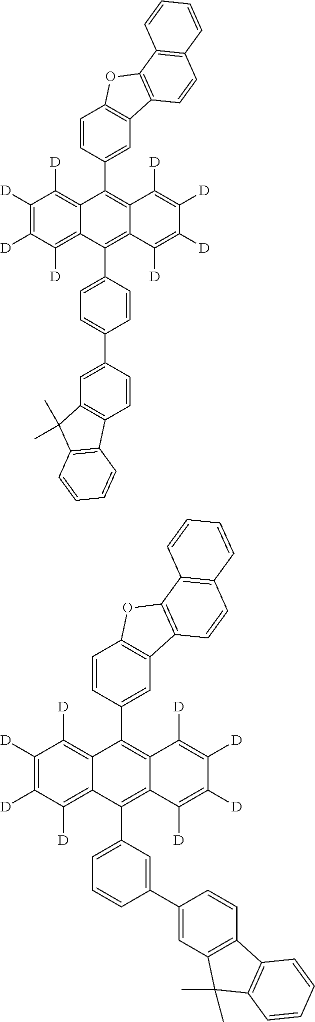 [SMILES: [2H]c1c([2H])c([2H])c2c(-c3ccc4oc5c6ccccc6ccc5c4c3)c3c([2H])c([2H])c([2H])c([2H])c3c(-c3ccc(-c4ccc5c(c4)C(C)(C)c4ccccc4-5)cc3)c2c1[2H] ; [2H]c1c([2H])c([2H])c2c(-c3ccc4oc5c6ccccc6ccc5c4c3)c3c([2H])c([2H])c([2H])c([2H])c3c(-c3cccc(-c4ccc5c(c4)C(C)(C)c4ccccc4-5)c3)c2c1[2H]]

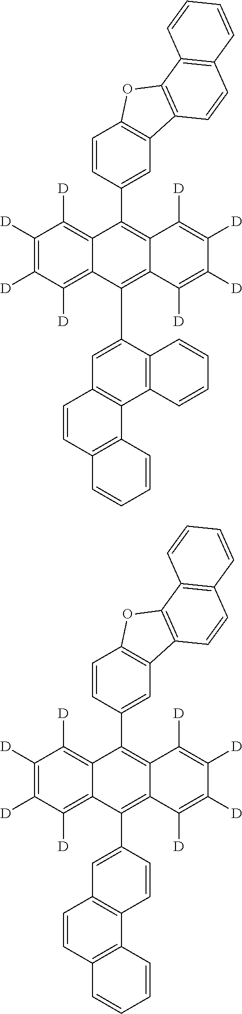 [SMILES: [2H]c1c([2H])c([2H])c2c(-c3cc4ccc5ccccc5c4c4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc4c(ccc5ccccc54)c3)c2c1[2H]]

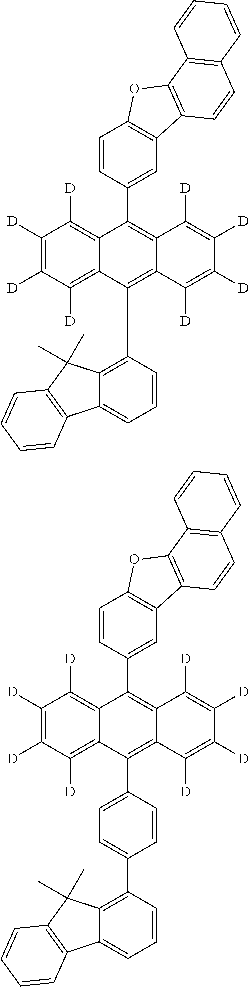 [SMILES: [2H]c1c([2H])c([2H])c2c(-c3cccc4c3C(C)(C)c3ccccc3-4)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4cccc5c4C(C)(C)c4ccccc4-5)cc3)c2c1[2H]]

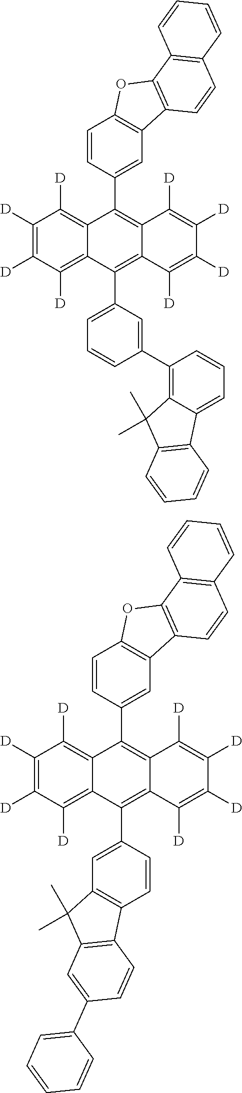

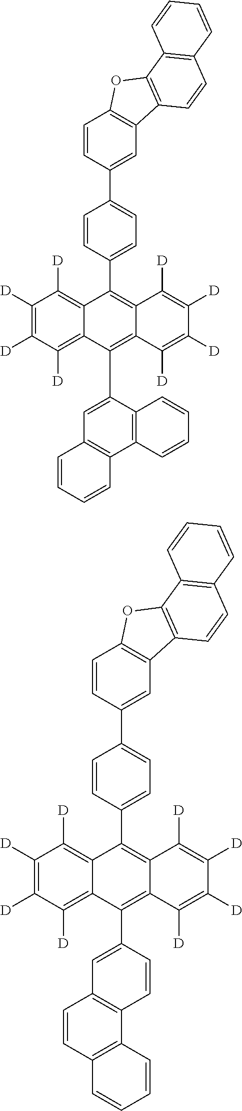 [SMILES: [2H]c1c([2H])c([2H])c2c(-c3cc4ccccc4c4ccccc34)c3c([2H])c([2H])c([2H])c([2H])c3c(-c3ccc(-c4ccc5oc6c7ccccc7ccc6c5c4)cc3)c2c1[2H] ; [2H]c1c([2H])c([2H])c2c(-c3ccc4c(ccc5ccccc54)c3)c3c([2H])c([2H])c([2H])c([2H])c3c(-c3ccc(-c4ccc5oc6c7ccccc7ccc6c5c4)cc3)c2c1[2H]]

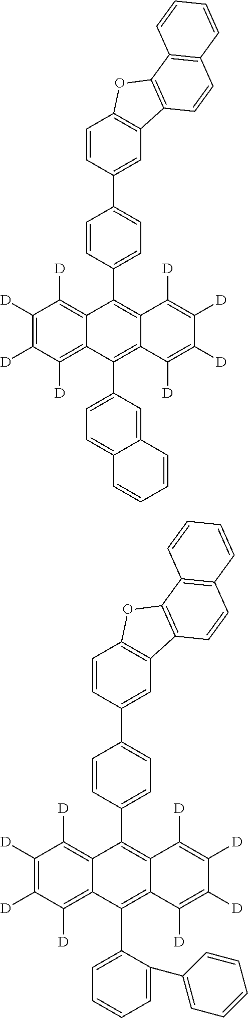

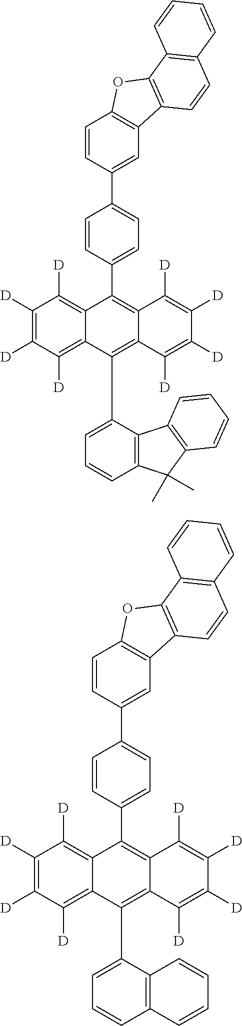

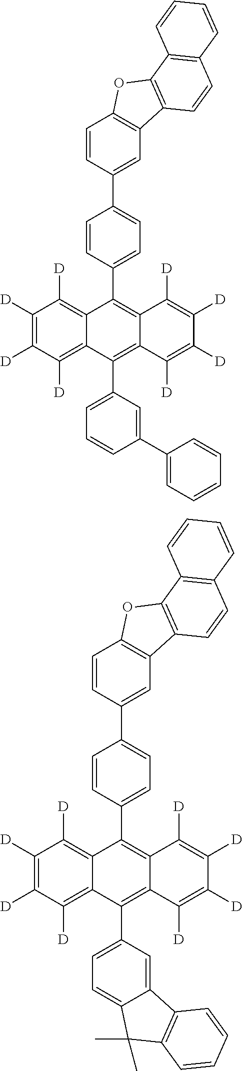

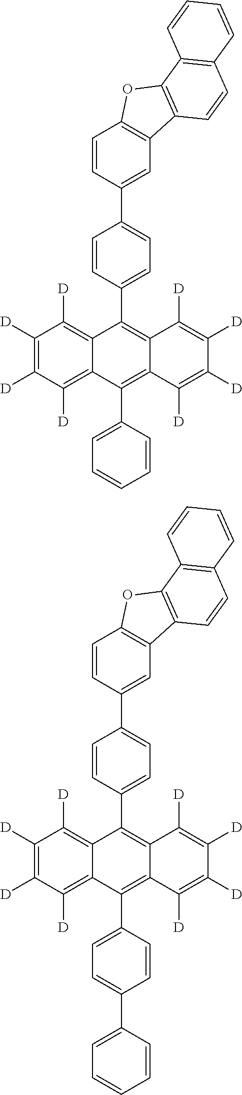 [SMILES: [2H]c1c([2H])c([2H])c2c(-c3ccc(-c4ccc5oc6c7ccccc7ccc6c5c4)cc3)c3c([2H])c([2H])c([2H])c([2H])c3c(-c3ccccc3)c2c1[2H] ; [2H]c1c([2H])c([2H])c2c(-c3ccc(-c4ccc5oc6c7ccccc7ccc6c5c4)cc3)c3c([2H])c([2H])c([2H])c([2H])c3c(-c3ccc(-c4ccccc4)cc3)c2c1[2H]]

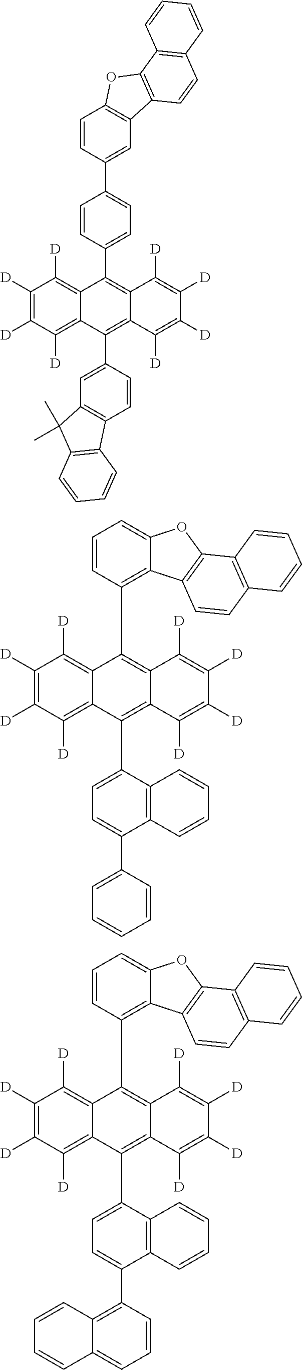

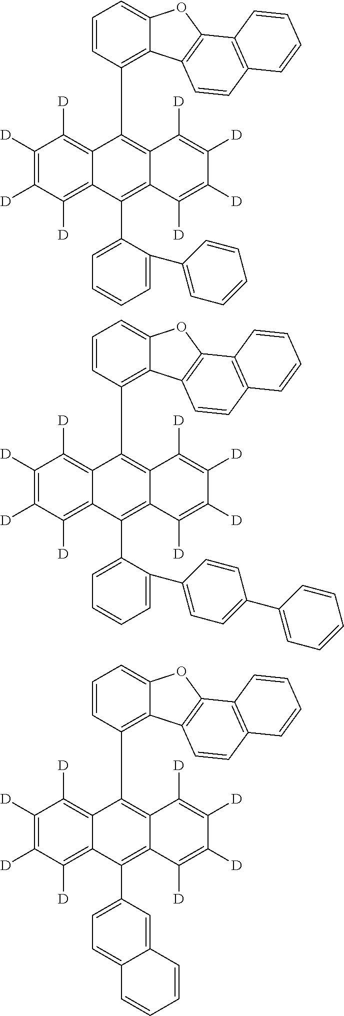

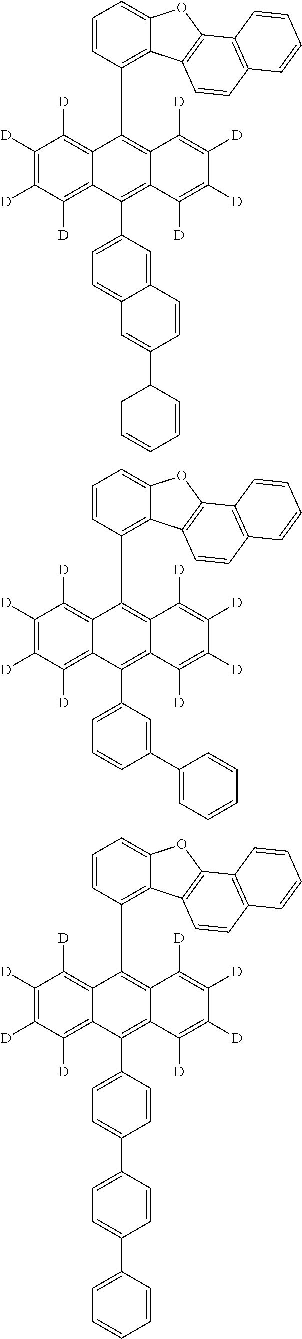

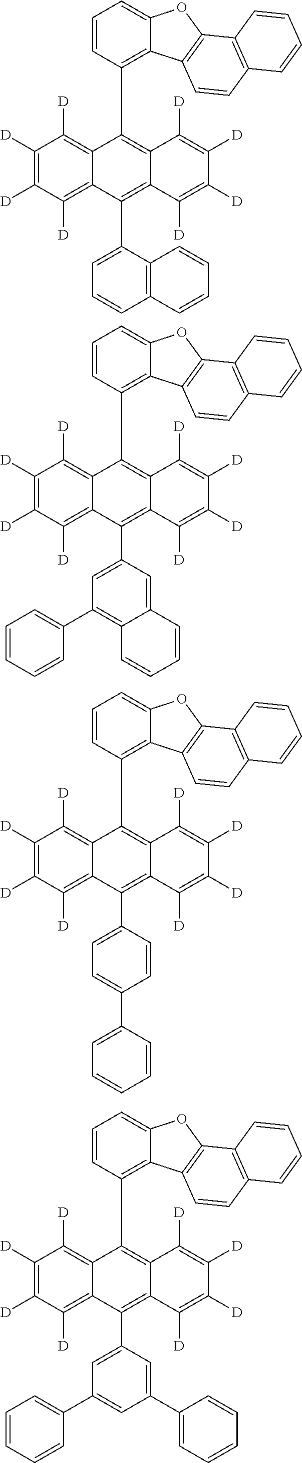

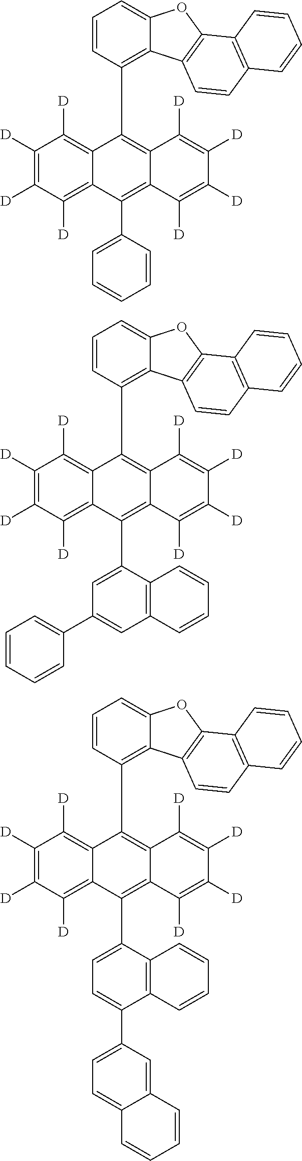

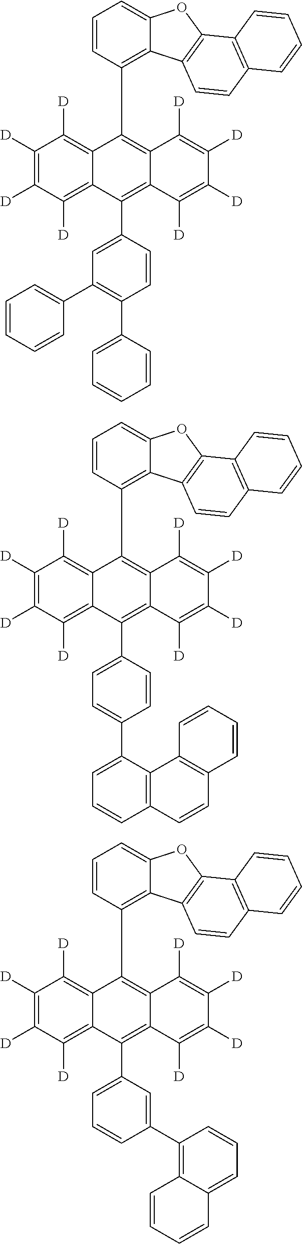

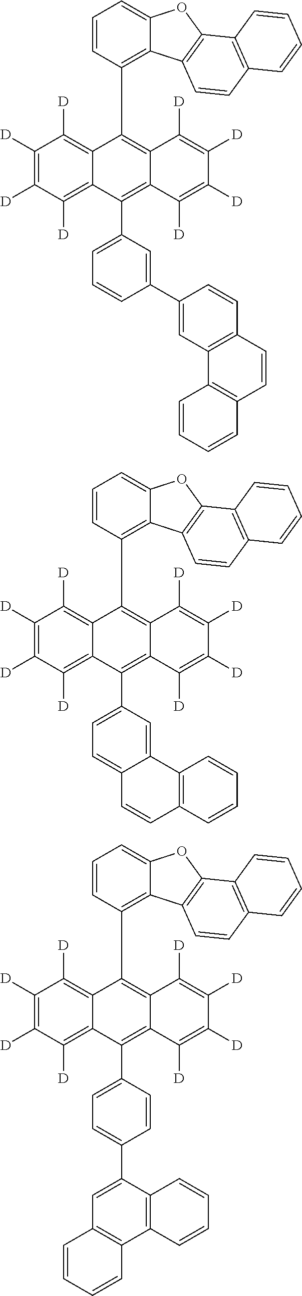

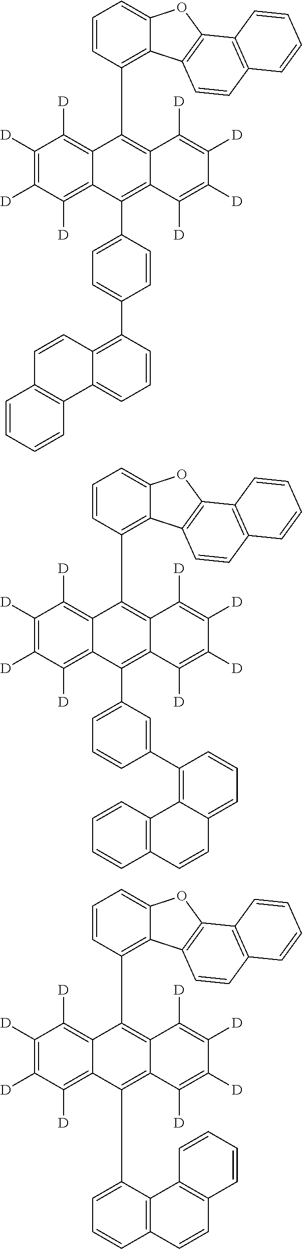

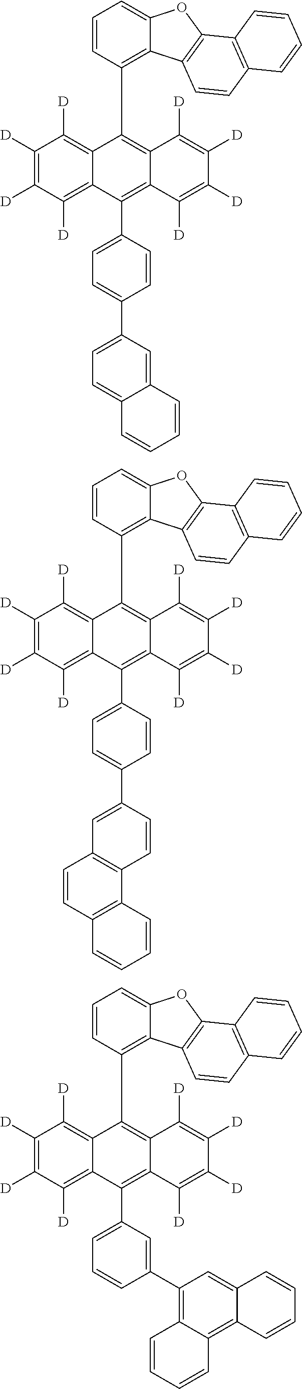

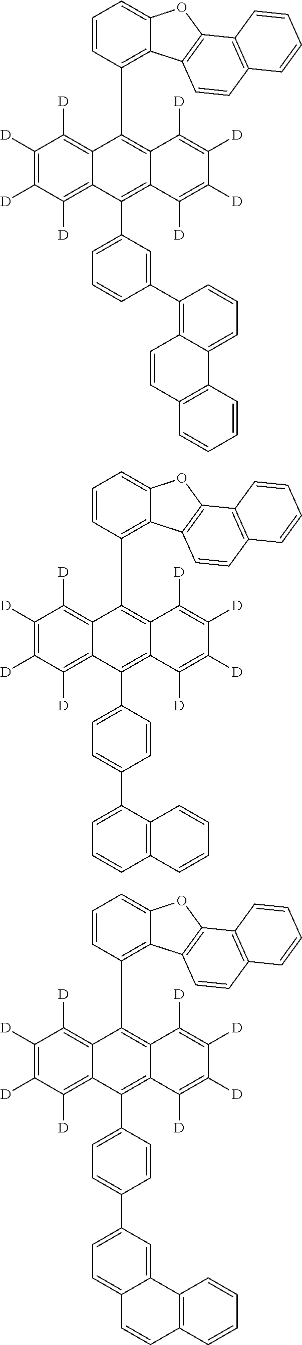

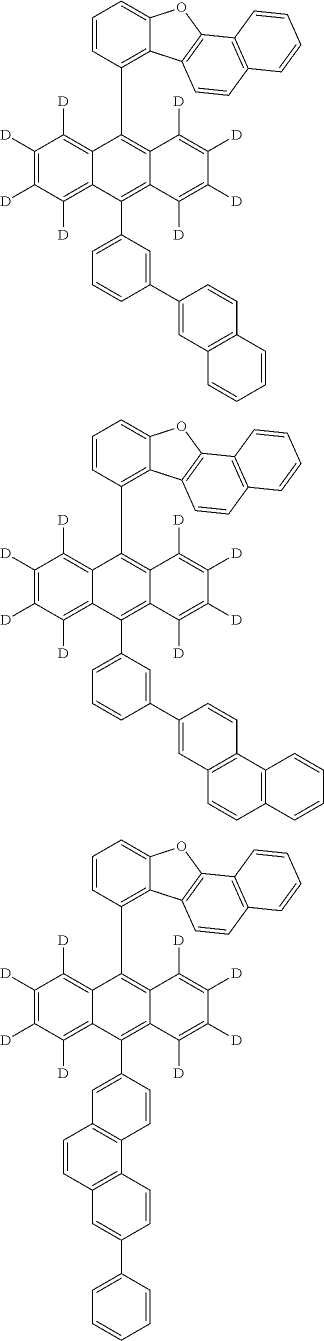

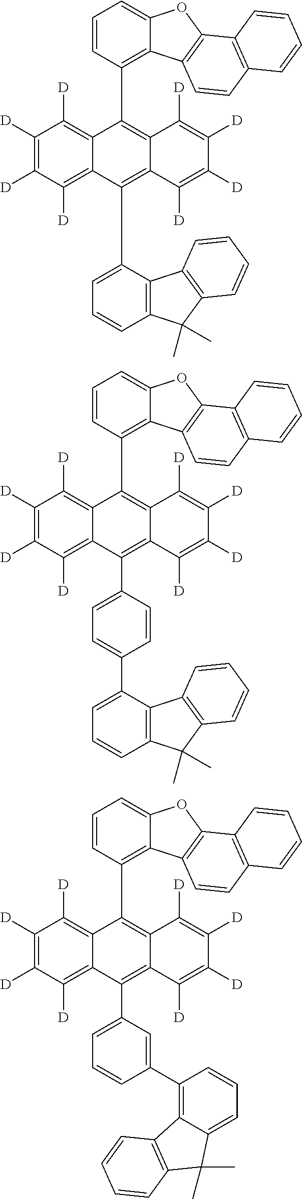

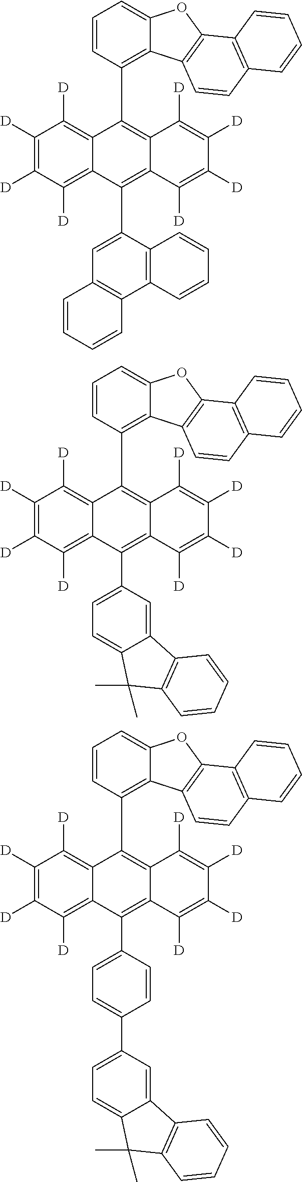

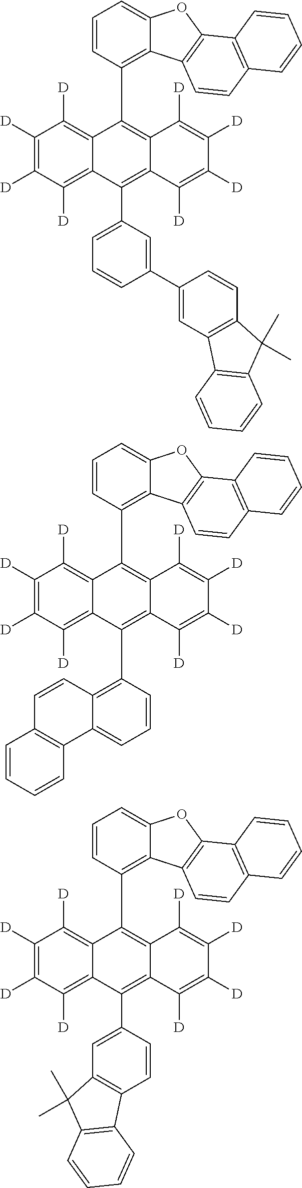

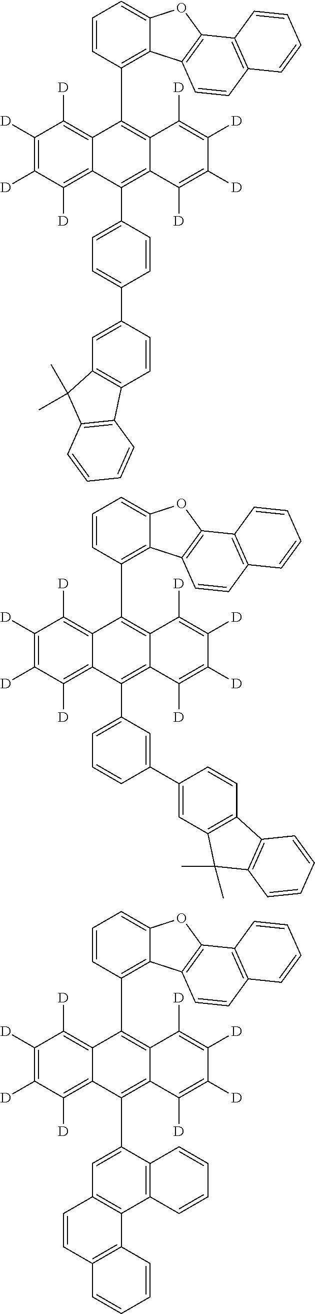

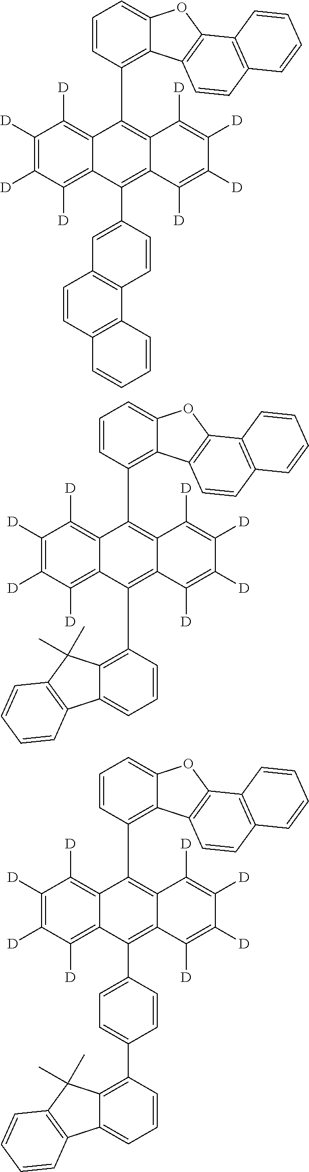

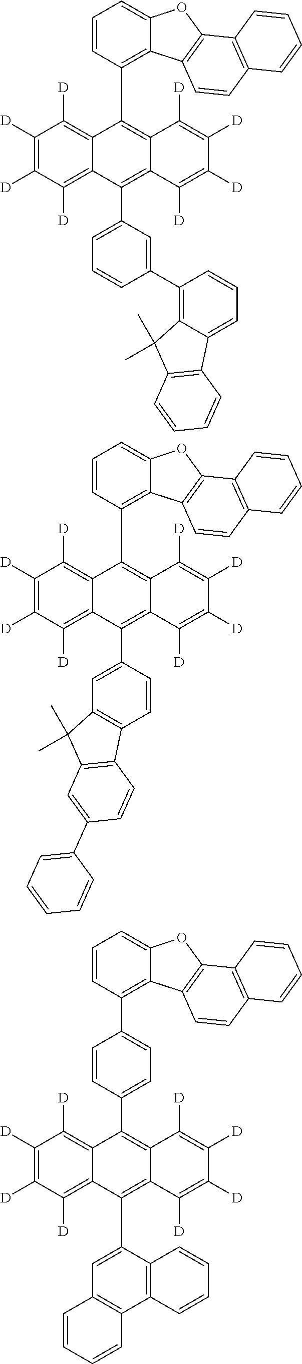

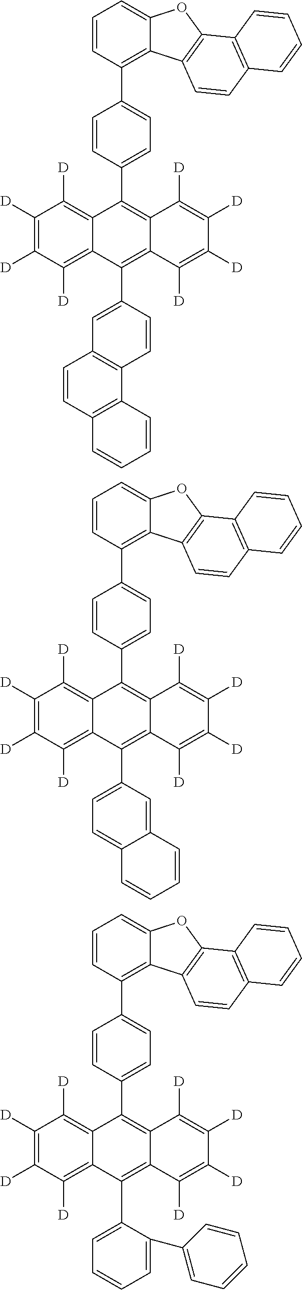

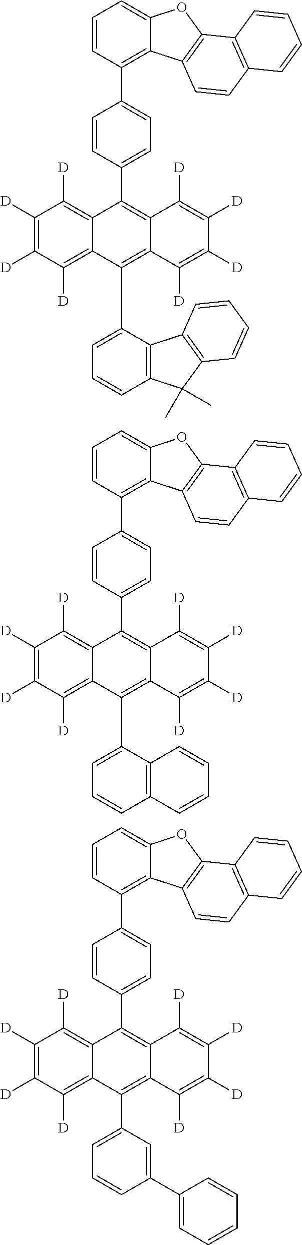

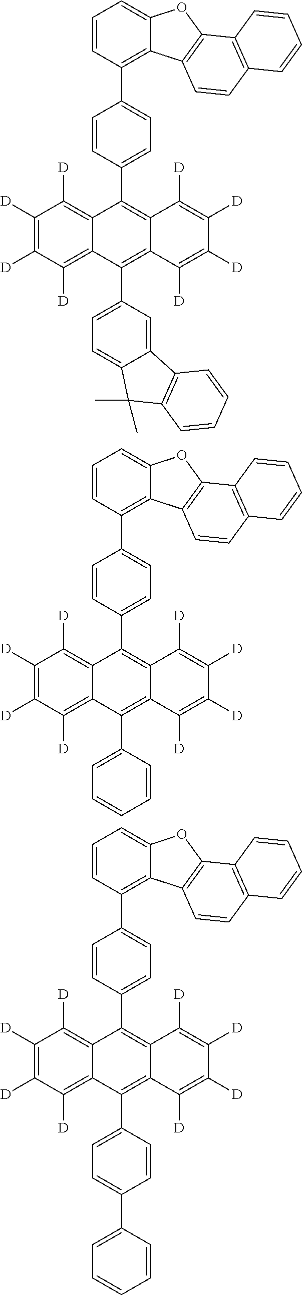

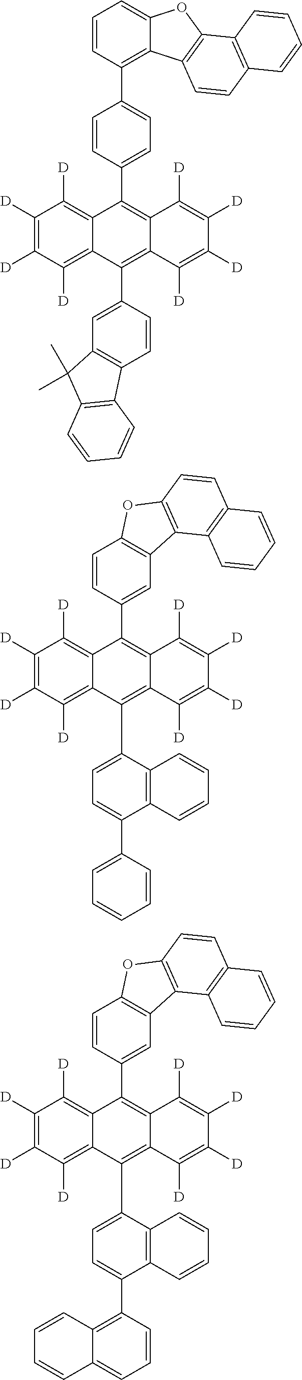

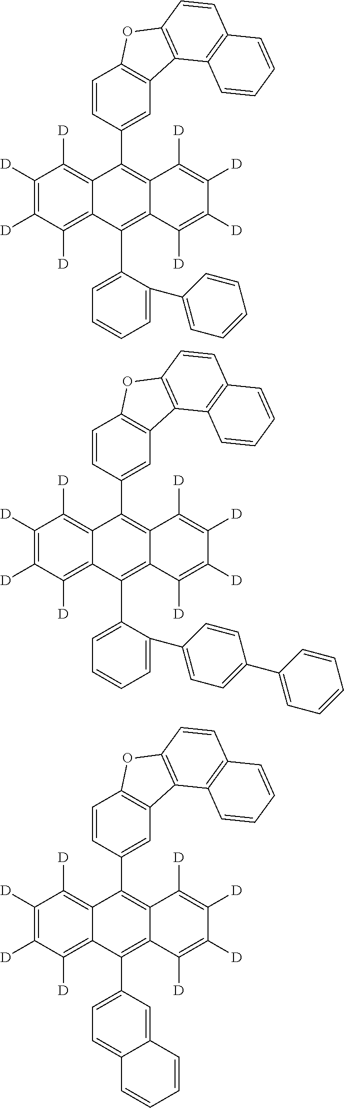

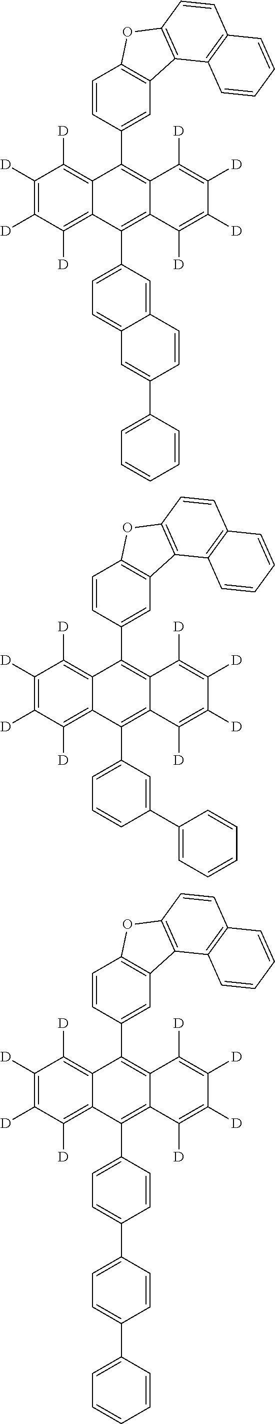

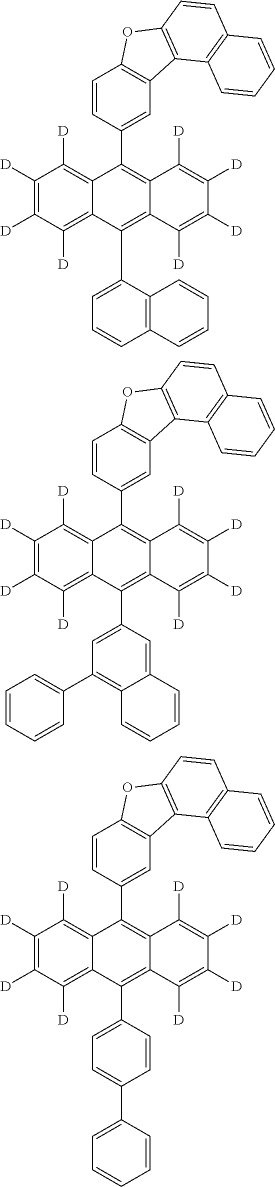

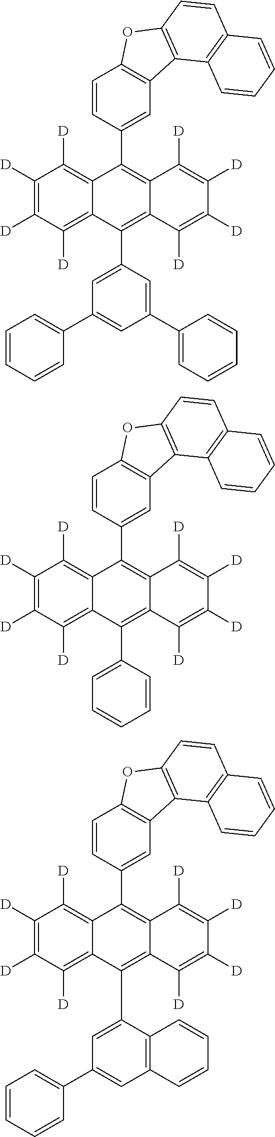

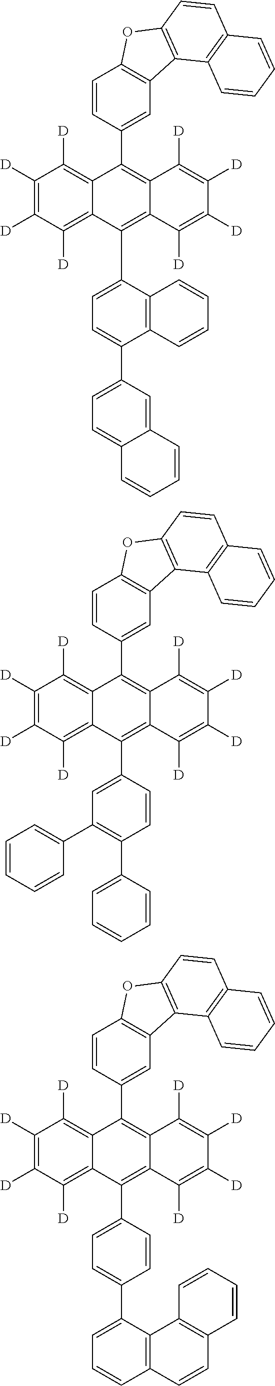

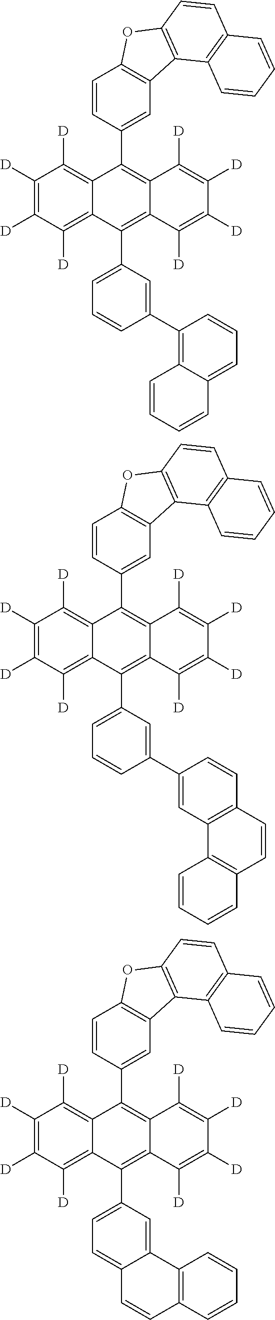

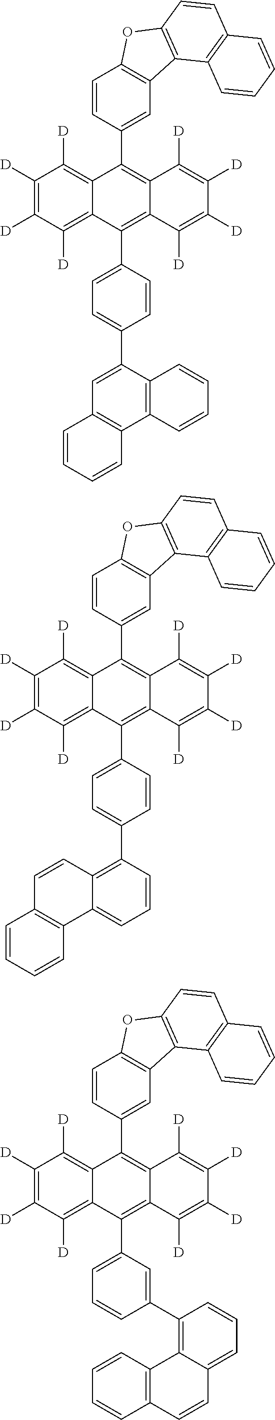 [SMILES: [2H]c1c([2H])c([2H])c2c(-c3ccc4oc5ccc6ccccc6c5c4c3)c3c([2H])c([2H])c([2H])c([2H])c3c(-c3ccc(-c4cc5ccccc5c5ccccc45)cc3)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4cccc5c4ccc4ccccc45)cc3)c2c1[2H] ; [2H]c1c([2H])c([2H])c2c(-c3ccc4oc5ccc6ccccc6c5c4c3)c3c([2H])c([2H])c([2H])c([2H])c3c(-c3cccc(-c4cccc5ccc6ccccc6c45)c3)c2c1[2H]]

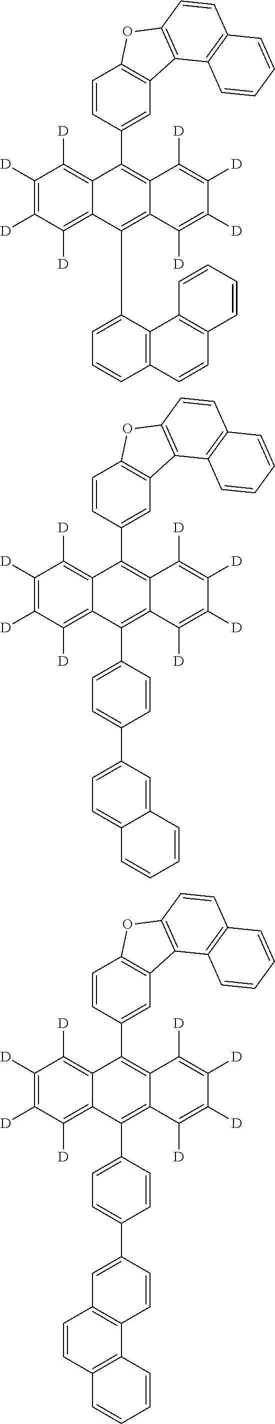

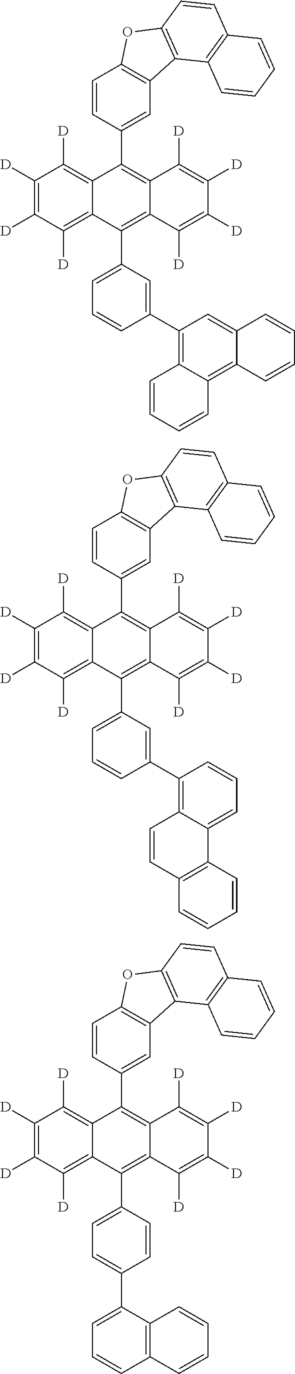

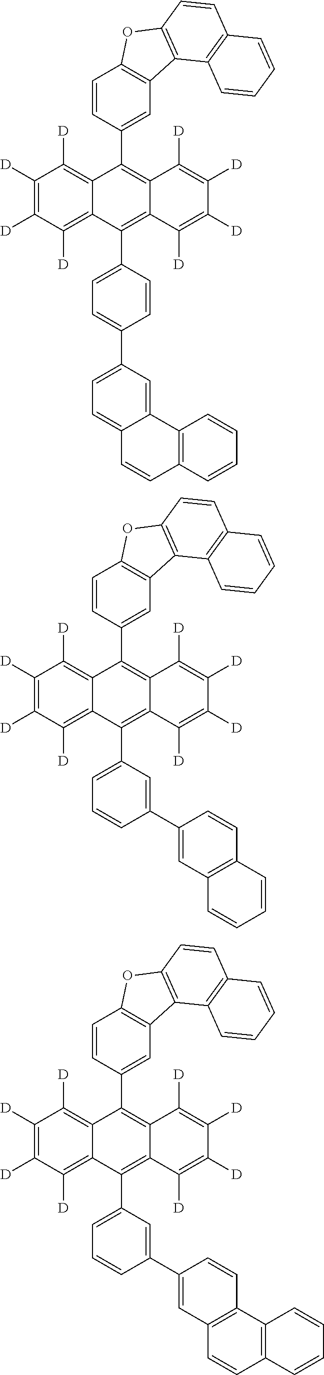

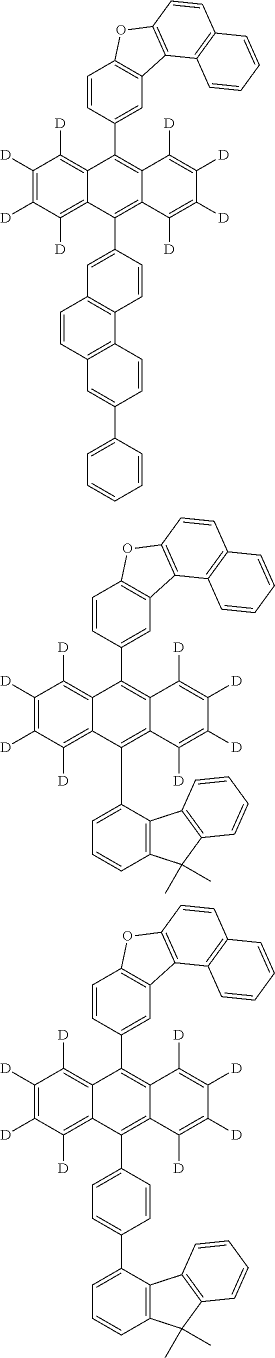

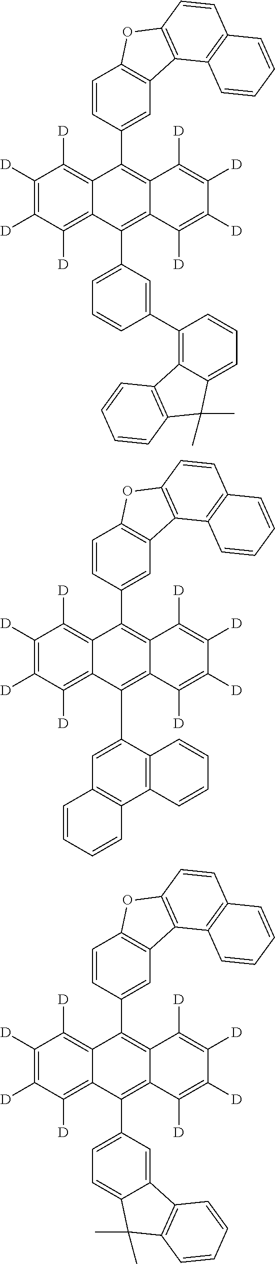

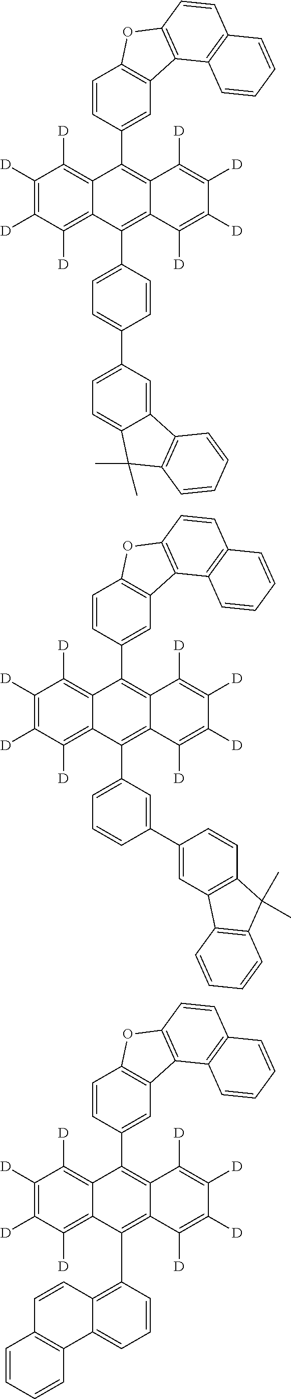

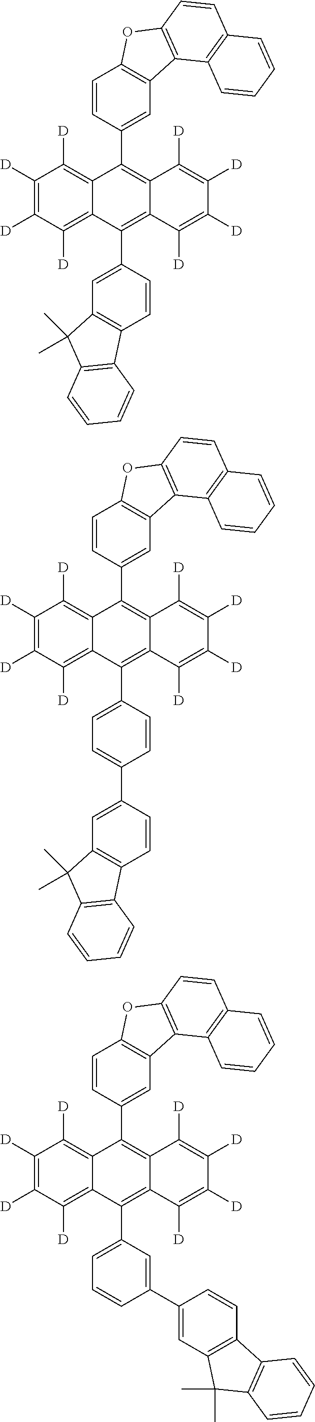 [SMILES: [2H]c1c([2H])c([2H])c2c(-c3ccc4oc5ccc6ccccc6c5c4c3)c3c([2H])c([2H])c([2H])c([2H])c3c(-c3ccc4c(c3)C(C)(C)c3ccccc3-4)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4ccc5c(c4)C(C)(C)c4ccccc4-5)cc3)c2c1[2H] ; [2H]c1c([2H])c([2H])c2c(-c3ccc4oc5ccc6ccccc6c5c4c3)c3c([2H])c([2H])c([2H])c([2H])c3c(-c3cccc(-c4ccc5c(c4)C(C)(C)c4ccccc4-5)c3)c2c1[2H]]

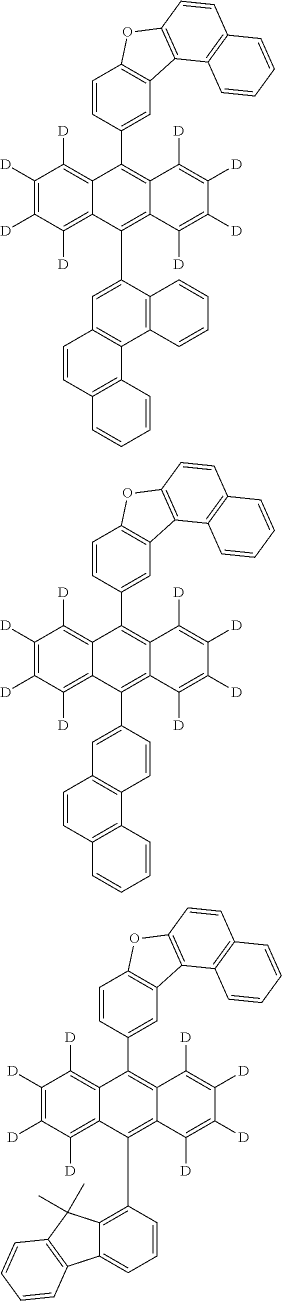

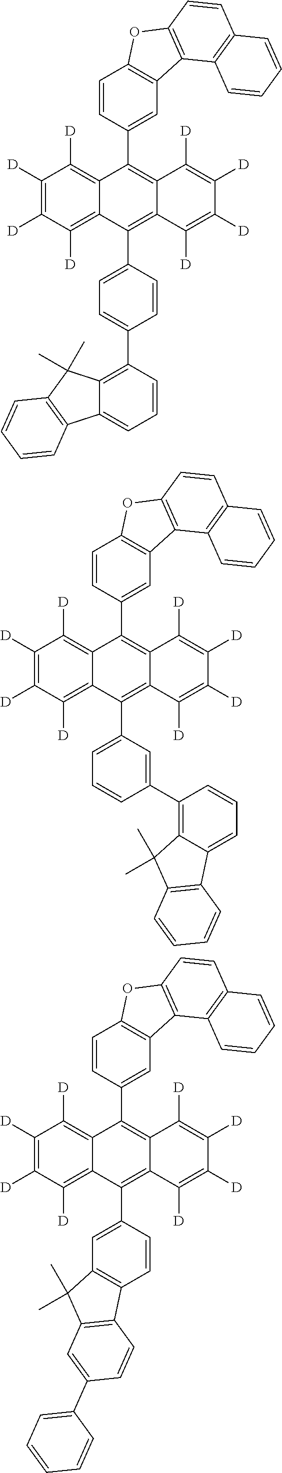

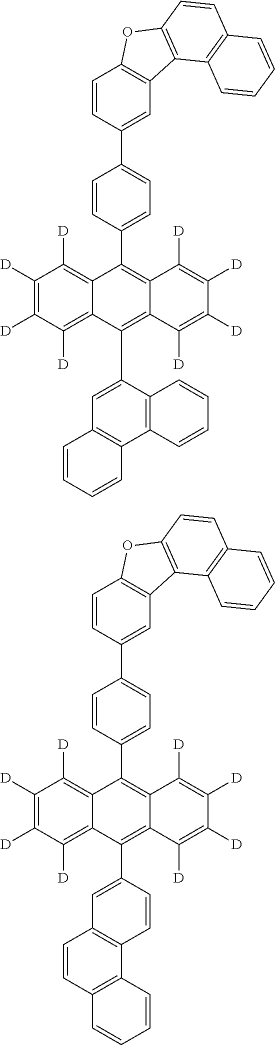 [SMILES: [2H]c1c([2H])c([2H])c2c(-c3cc4ccccc4c4ccccc34)c3c([2H])c([2H])c([2H])c([2H])c3c(-c3ccc(-c4ccc5oc6ccc7ccccc7c6c5c4)cc3)c2c1[2H] ; [2H]c1c([2H])c([2H])c2c(-c3ccc4c(ccc5ccccc54)c3)c3c([2H])c([2H])c([2H])c([2H])c3c(-c3ccc(-c4ccc5oc6ccc7ccccc7c6c5c4)cc3)c2c1[2H]]

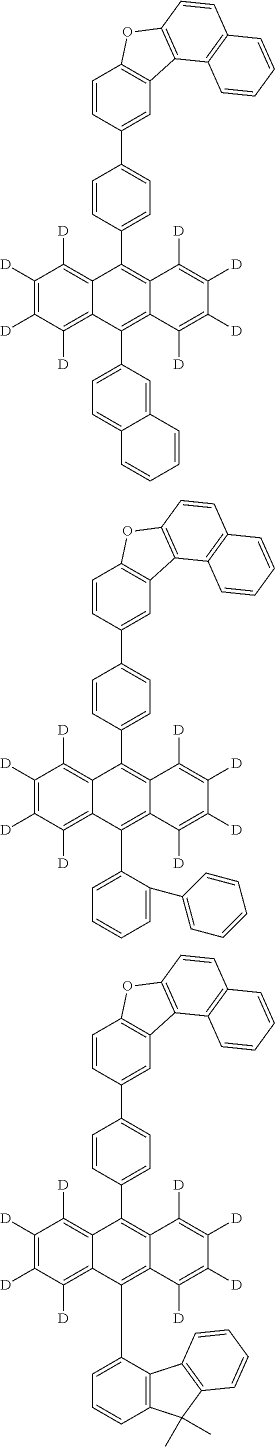

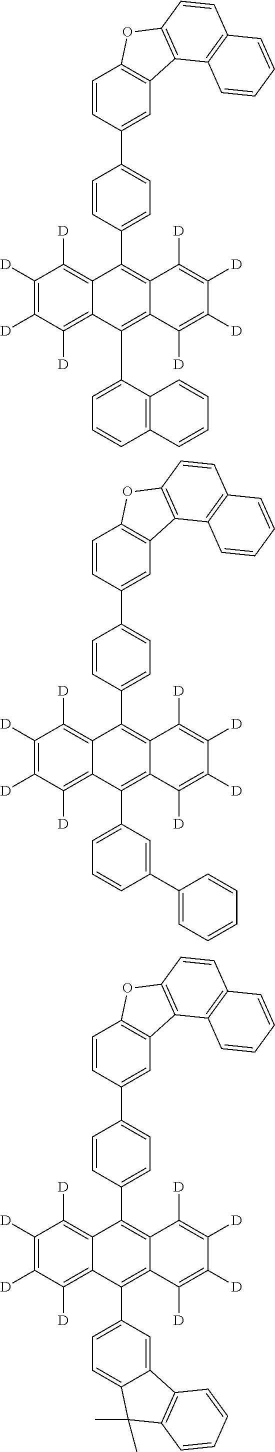

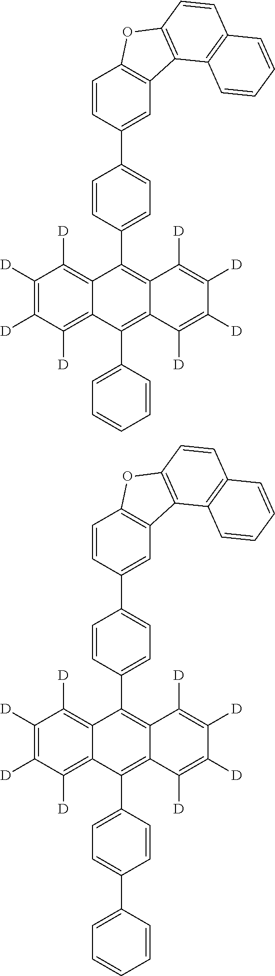

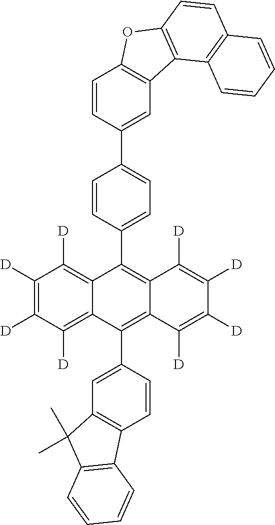 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4ccc5oc6ccc7ccccc7c6c5c4)cc3)c2c1[2H]]

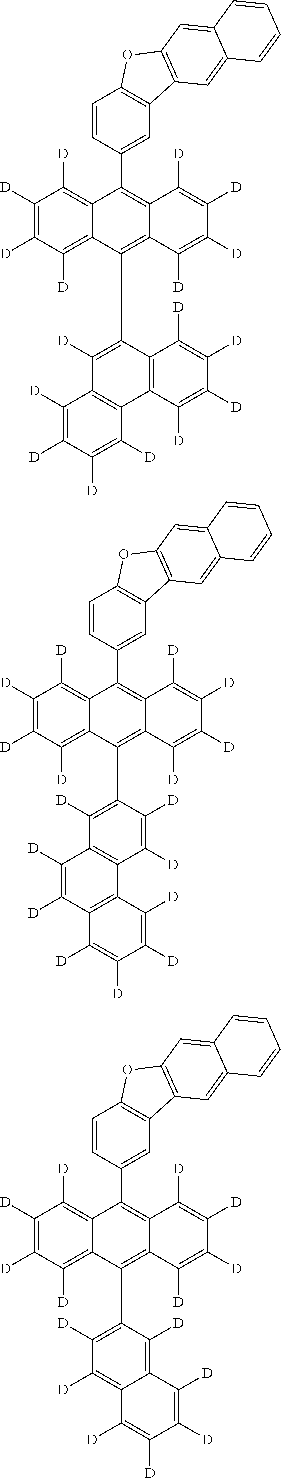 [SMILES: [2H]c1c([2H])c([2H])c2c(-c3c([2H])c4c([2H])c([2H])c([2H])c([2H])c4c4c([2H])c([2H])c([2H])c([2H])c34)c3c([2H])c([2H])c([2H])c([2H])c3c(-c3ccc4oc5cc6ccccc6cc5c4c3)c2c1[2H] ; [2H]c1c([2H])c([2H])c2c(-c3c([2H])c([2H])c4c(c3[2H])c([2H])c([2H])c3c([2H])c([2H])c([2H])c([2H])c34)c3c([2H])c([2H])c([2H])c([2H])c3c(-c3ccc4oc5cc6ccccc6cc5c4c3)c2c1[2H] ; [2H]c1c([2H])c([2H])c2c([2H])c(-c3c4c([2H])c([2H])c([2H])c([2H])c4c(-c4ccc5oc6cc7ccccc7cc6c5c4)c4c([2H])c([2H])c([2H])c([2H])c34)c([2H])c([2H])c2c1[2H]]

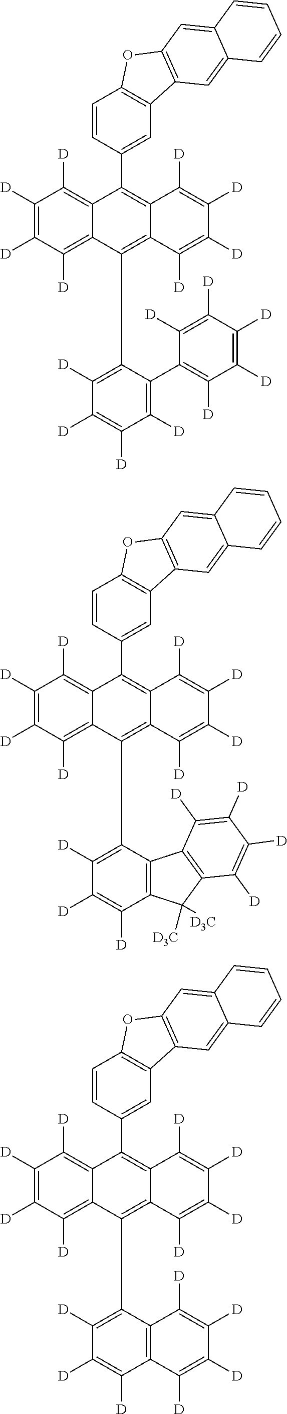

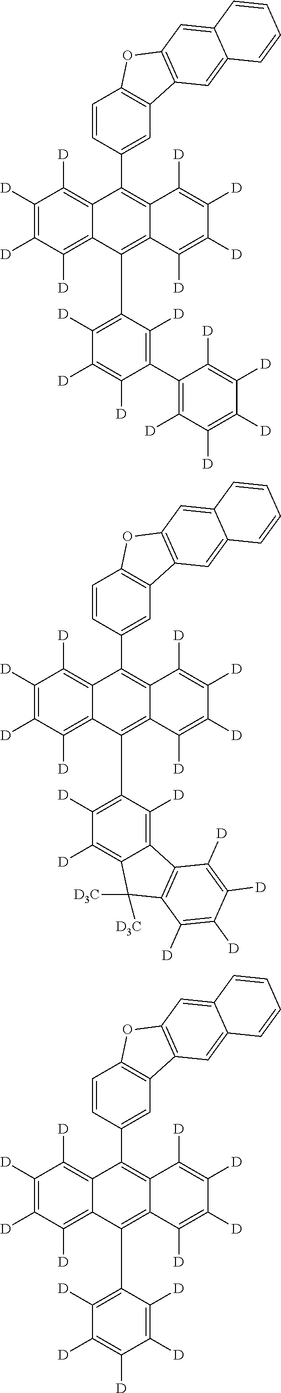

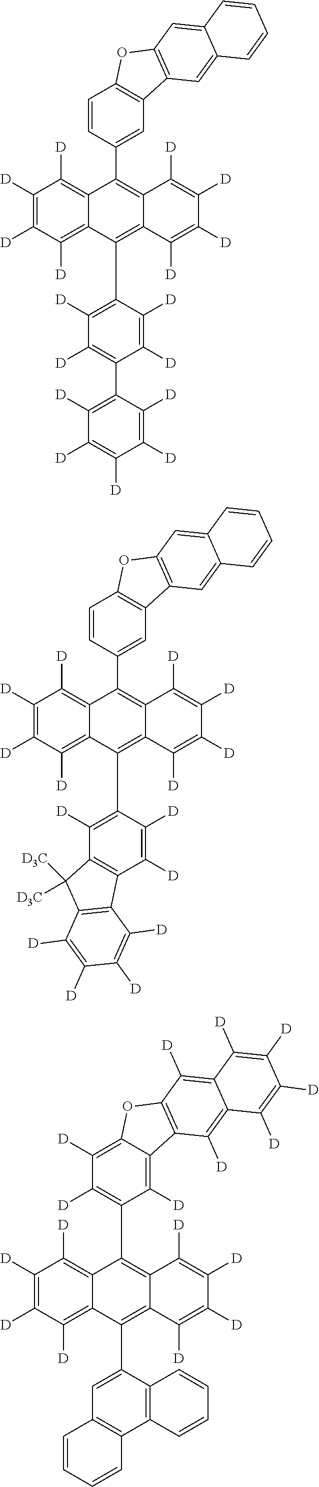

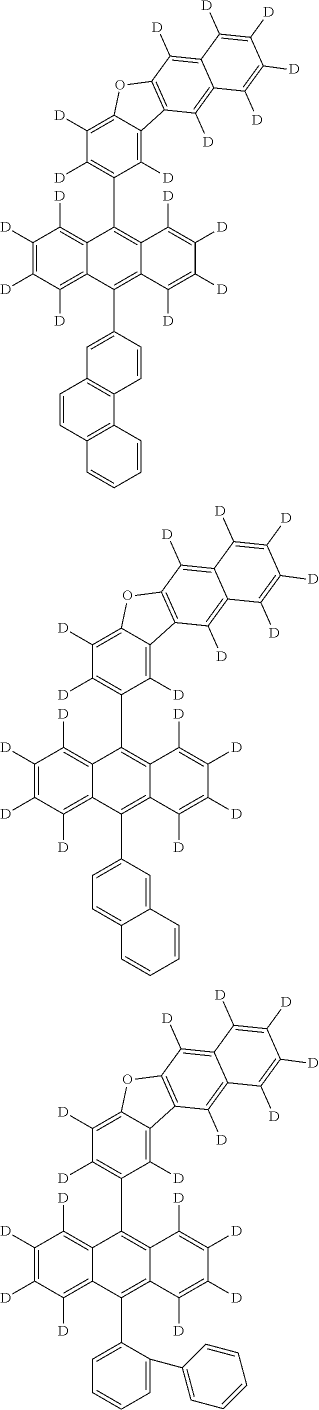

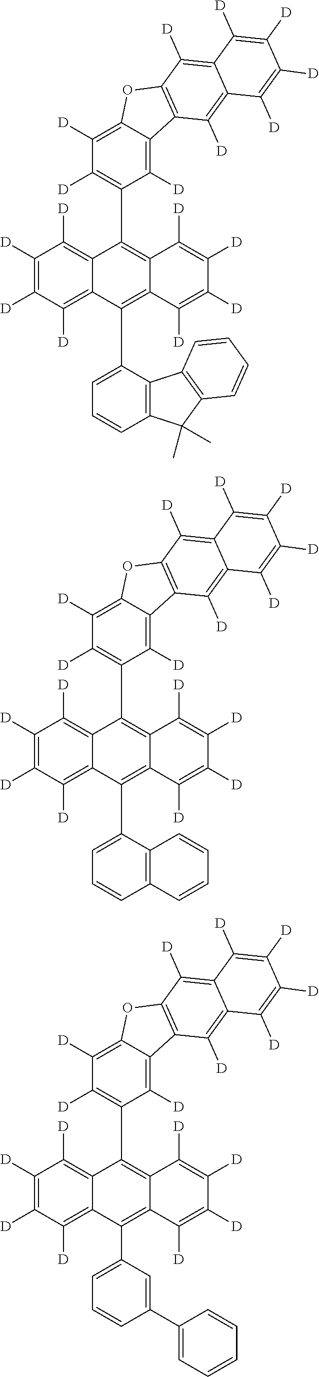

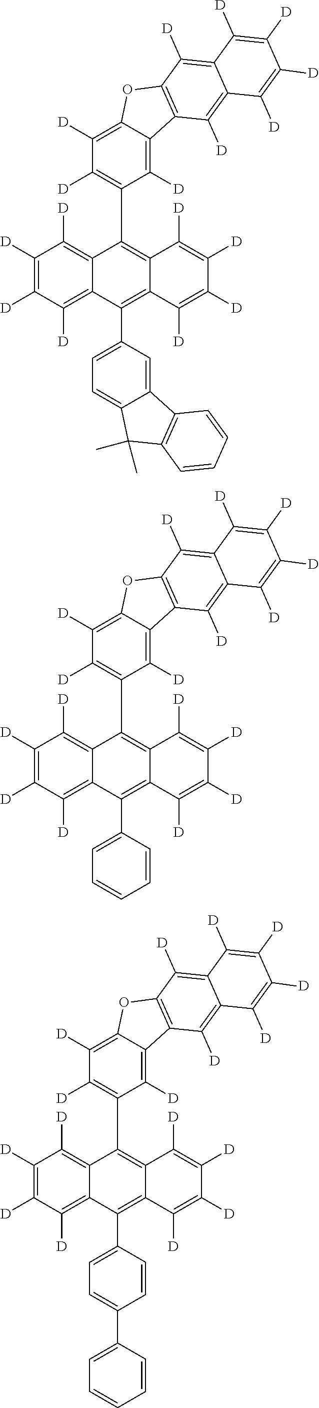

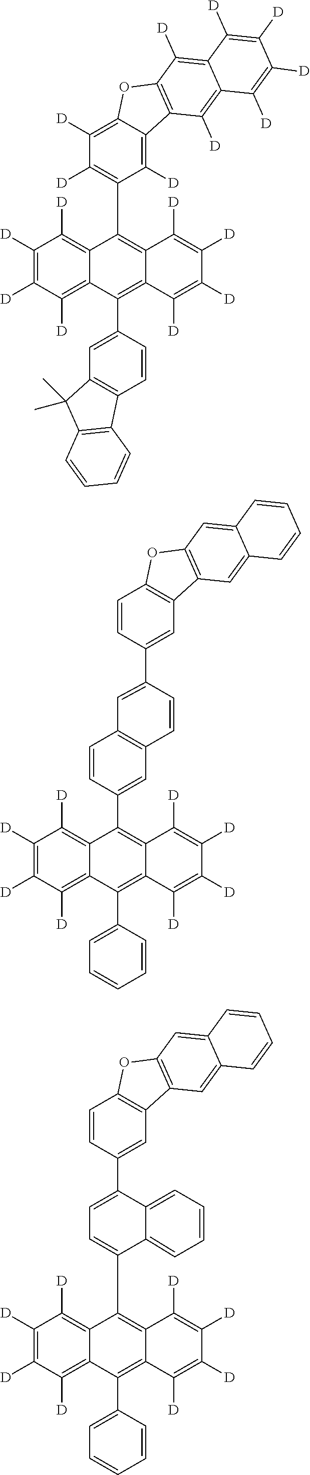 [SMILES: [2H]c1c(-c2c3c([2H])c([2H])c([2H])c([2H])c3c(-c3ccc4c(c3)C(C)(C)c3ccccc3-4)c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c2c(-c3ccc4cc(-c5ccc6oc7cc8ccccc8cc7c6c5)ccc4c3)c3c([2H])c([2H])c([2H])c([2H])c3c(-c3ccccc3)c2c1[2H] ; [2H]c1c([2H])c([2H])c2c(-c3ccc(-c4ccc5oc6cc7ccccc7cc6c5c4)c4ccccc34)c3c([2H])c([2H])c([2H])c([2H])c3c(-c3ccccc3)c2c1[2H]]

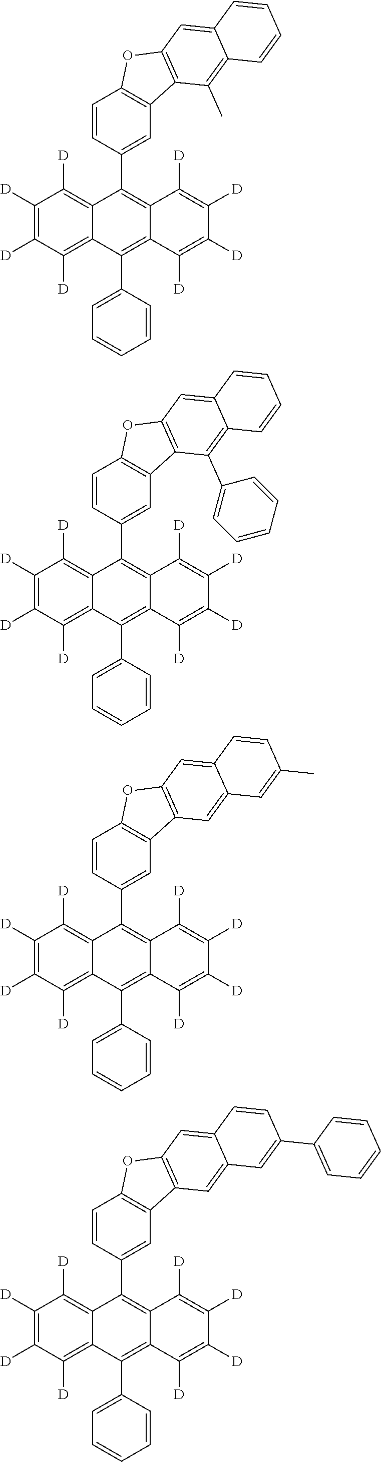 [SMILES: [2H]c1c([2H])c([2H])c2c(-c3ccc4oc5cc6ccccc6c(C)c5c4c3)c3c([2H])c([2H])c([2H])c([2H])c3c(-c3ccccc3)c2c1[2H] ; [2H]c1c([2H])c([2H])c2c(-c3ccc4oc5cc6ccccc6c(-c6ccccc6)c5c4c3)c3c([2H])c([2H])c([2H])c([2H])c3c(-c3ccccc3)c2c1[2H] ; [2H]c1c([2H])c([2H])c2c(-c3ccc4oc5cc6ccc(C)cc6cc5c4c3)c3c([2H])c([2H])c([2H])c([2H])c3c(-c3ccccc3)c2c1[2H] ; [2H]c1c([2H])c([2H])c2c(-c3ccc4oc5cc6ccc(-c7ccccc7)cc6cc5c4c3)c3c([2H])c([2H])c([2H])c([2H])c3c(-c3ccccc3)c2c1[2H]]

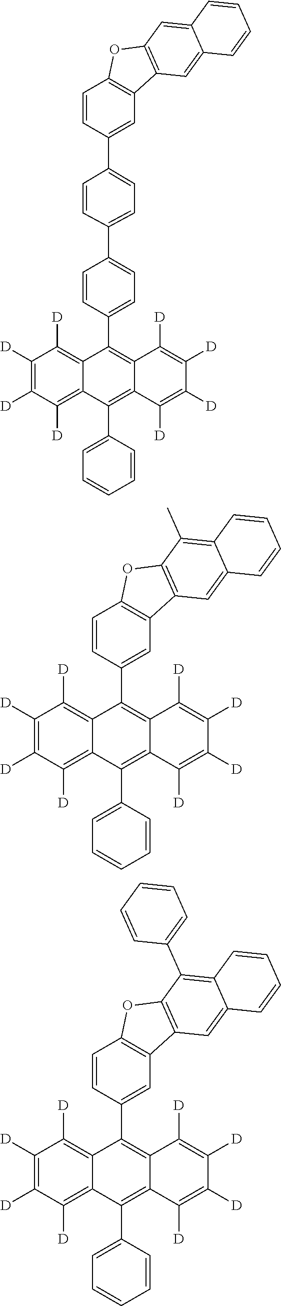

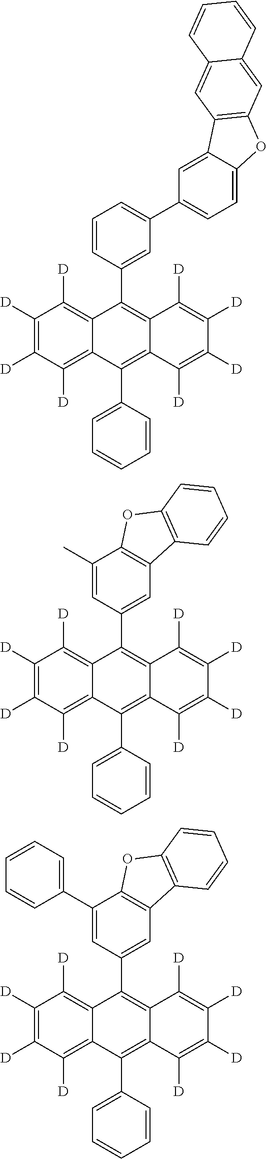

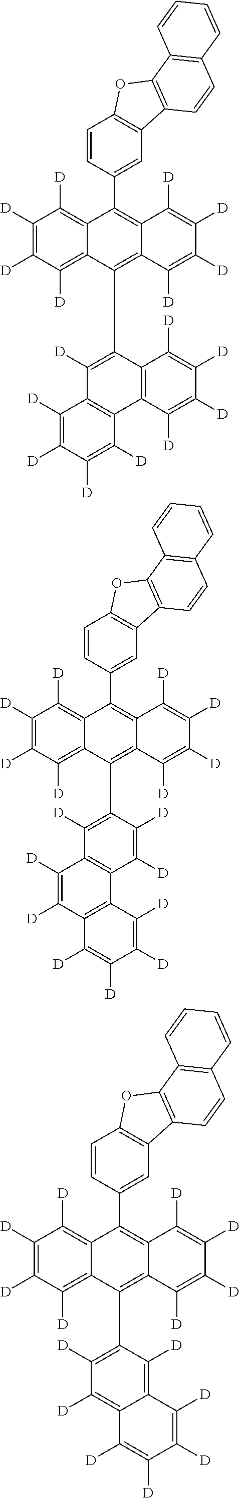 [SMILES: [2H]c1c([2H])c([2H])c2c(-c3c([2H])c4c([2H])c([2H])c([2H])c([2H])c4c4c([2H])c([2H])c([2H])c([2H])c34)c3c([2H])c([2H])c([2H])c([2H])c3c(-c3ccc4oc5c6ccccc6ccc5c4c3)c2c1[2H] ; [2H]c1c([2H])c([2H])c2c(-c3c([2H])c([2H])c4c(c3[2H])c([2H])c([2H])c3c([2H])c([2H])c([2H])c([2H])c34)c3c([2H])c([2H])c([2H])c([2H])c3c(-c3ccc4oc5c6ccccc6ccc5c4c3)c2c1[2H] ; [2H]c1c([2H])c([2H])c2c([2H])c(-c3c4c([2H])c([2H])c([2H])c([2H])c4c(-c4ccc5oc6c7ccccc7ccc6c5c4)c4c([2H])c([2H])c([2H])c([2H])c34)c([2H])c([2H])c2c1[2H]]

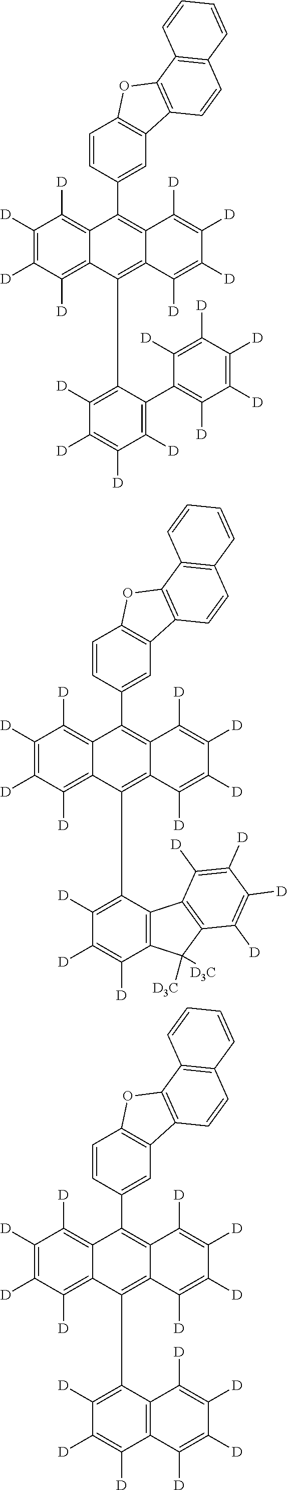

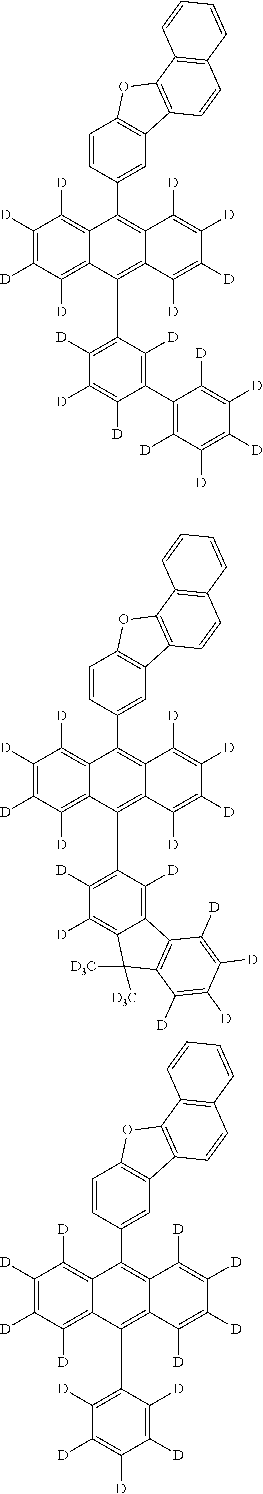

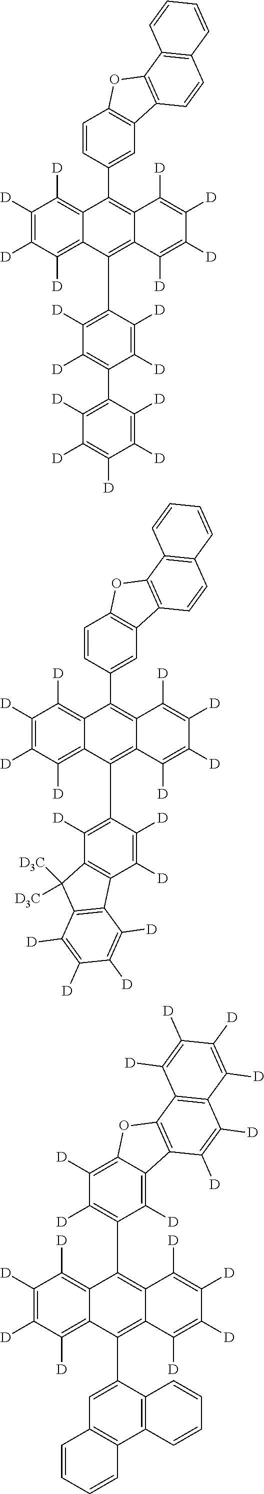

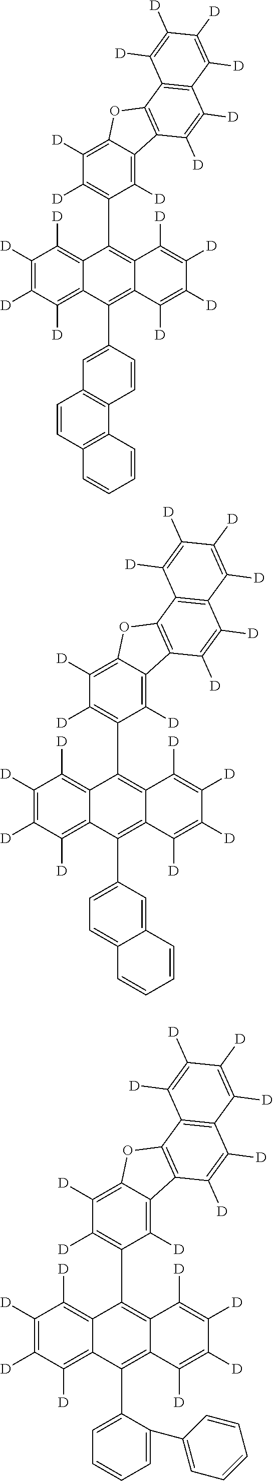

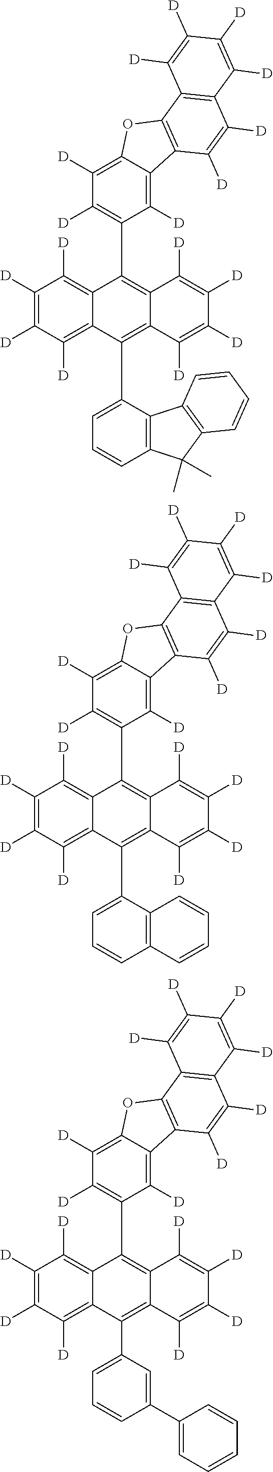

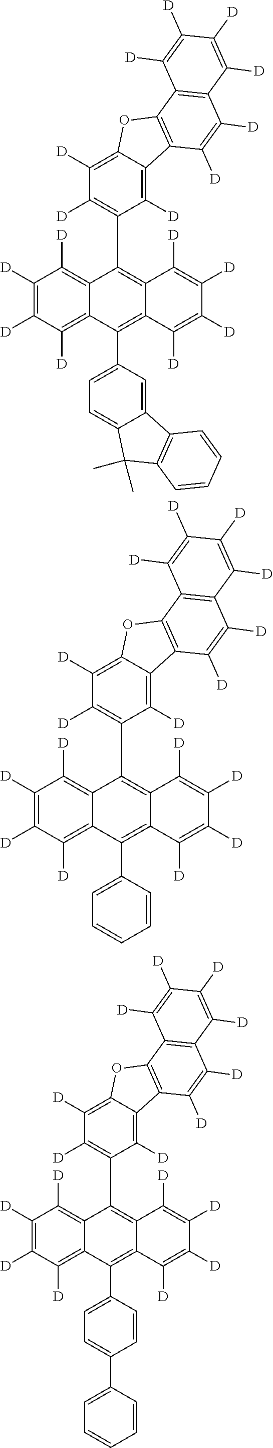

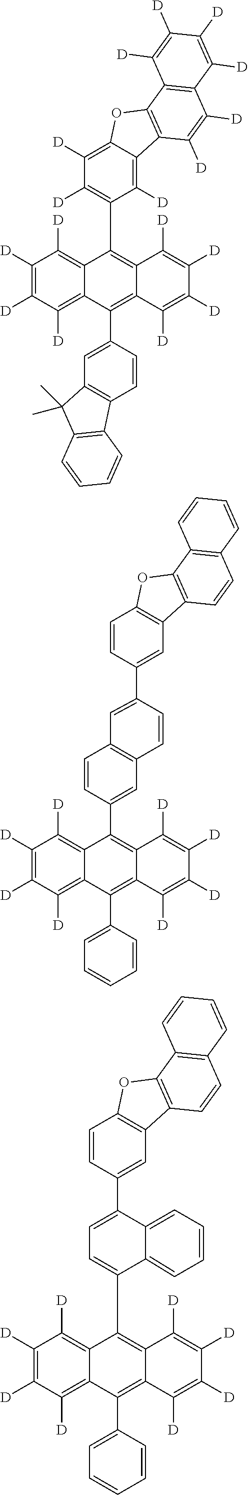 [SMILES: [2H]c1c(-c2c3c([2H])c([2H])c([2H])c([2H])c3c(-c3ccc4c(c3)C(C)(C)c3ccccc3-4)c3c([2H])c([2H])c([2H])c([2H])c23)c([2H])c2c(oc3c4c([2H])c([2H])c([2H])c([2H])c4c([2H])c([2H])c32)c1[2H] ; [2H]c1c([2H])c([2H])c2c(-c3ccc4cc(-c5ccc6oc7c8ccccc8ccc7c6c5)ccc4c3)c3c([2H])c([2H])c([2H])c([2H])c3c(-c3ccccc3)c2c1[2H] ; [2H]c1c([2H])c([2H])c2c(-c3ccc(-c4ccc5oc6c7ccccc7ccc6c5c4)c4ccccc34)c3c([2H])c([2H])c([2H])c([2H])c3c(-c3ccccc3)c2c1[2H]]

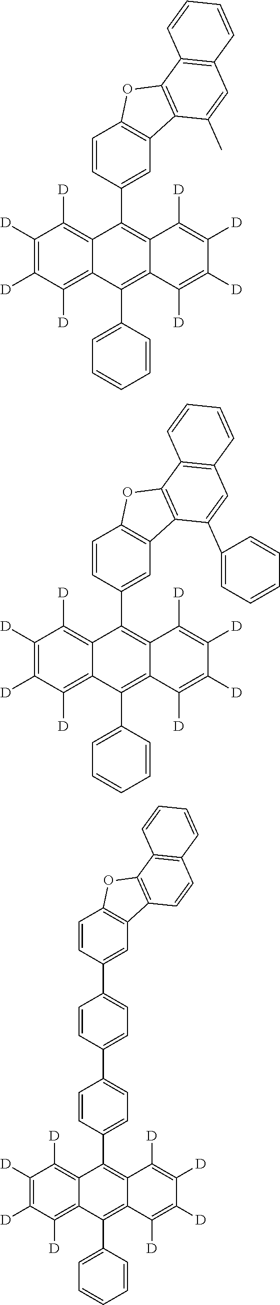

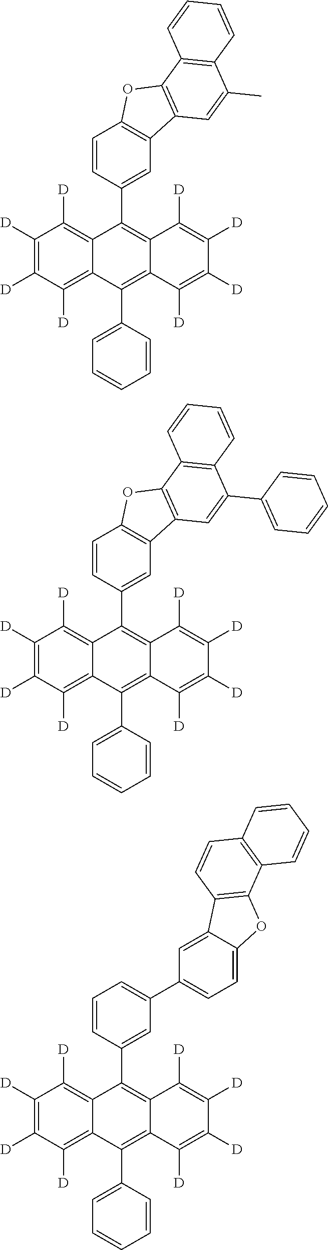 [SMILES: [2H]c1c([2H])c([2H])c2c(-c3ccc4oc5c6ccccc6c(C)cc5c4c3)c3c([2H])c([2H])c([2H])c([2H])c3c(-c3ccccc3)c2c1[2H] ; [2H]c1c([2H])c([2H])c2c(-c3ccc4oc5c6ccccc6c(-c6ccccc6)cc5c4c3)c3c([2H])c([2H])c([2H])c([2H])c3c(-c3ccccc3)c2c1[2H] ; [2H]c1c([2H])c([2H])c2c(-c3cccc(-c4ccc5oc6c7ccccc7ccc6c5c4)c3)c3c([2H])c([2H])c([2H])c([2H])c3c(-c3ccccc3)c2c1[2H]]

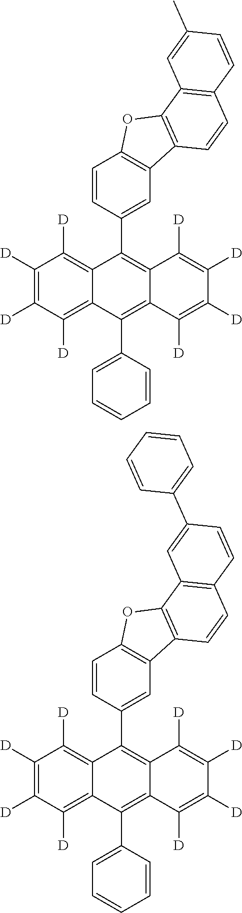

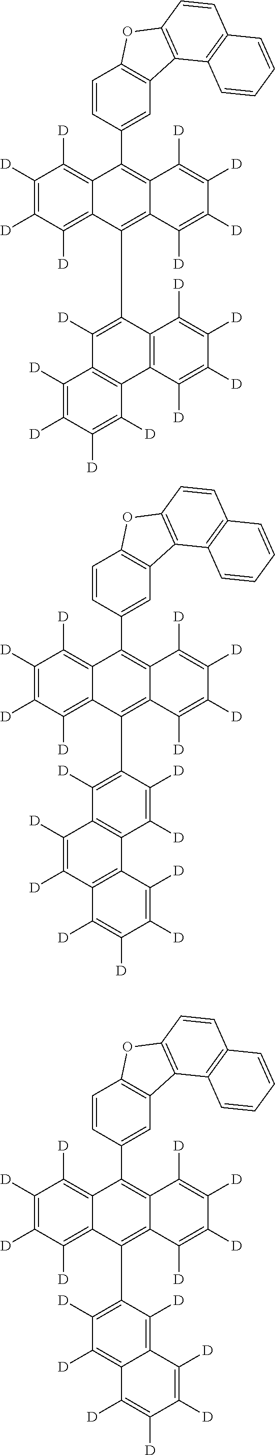 [SMILES: [2H]c1c([2H])c([2H])c2c(-c3c([2H])c4c([2H])c([2H])c([2H])c([2H])c4c4c([2H])c([2H])c([2H])c([2H])c34)c3c([2H])c([2H])c([2H])c([2H])c3c(-c3ccc4oc5ccc6ccccc6c5c4c3)c2c1[2H] ; [2H]c1c([2H])c([2H])c2c(-c3c([2H])c([2H])c4c(c3[2H])c([2H])c([2H])c3c([2H])c([2H])c([2H])c([2H])c34)c3c([2H])c([2H])c([2H])c([2H])c3c(-c3ccc4oc5ccc6ccccc6c5c4c3)c2c1[2H] ; [2H]c1c([2H])c([2H])c2c([2H])c(-c3c4c([2H])c([2H])c([2H])c([2H])c4c(-c4ccc5oc6ccc7ccccc7c6c5c4)c4c([2H])c([2H])c([2H])c([2H])c34)c([2H])c([2H])c2c1[2H]]

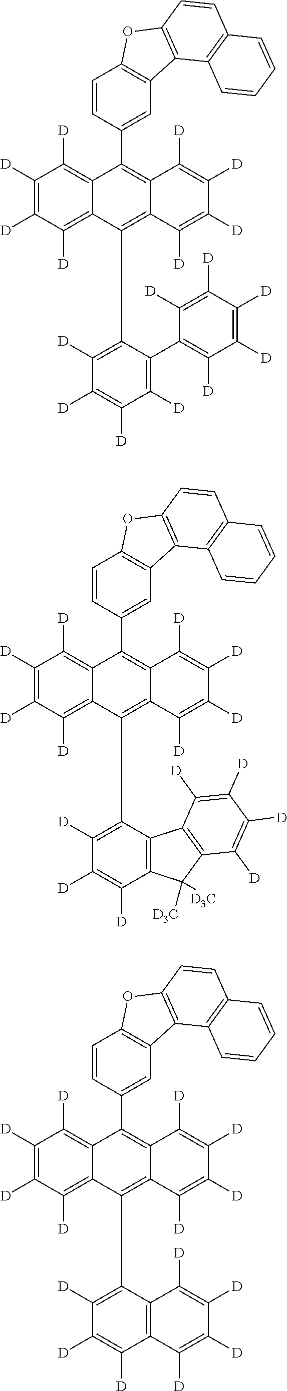

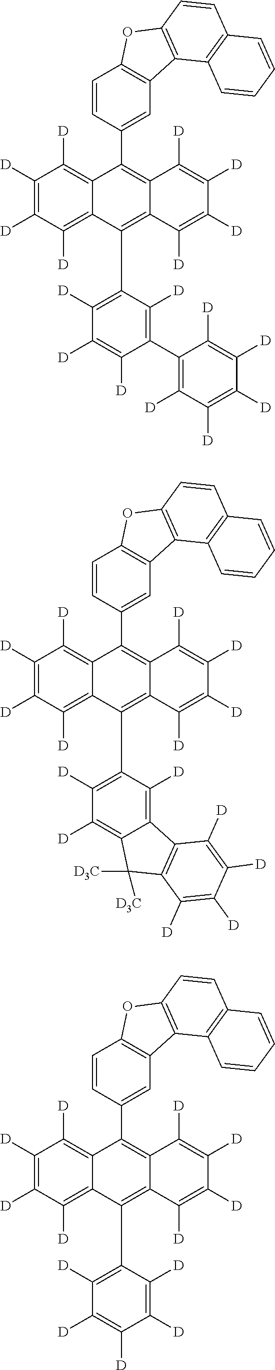

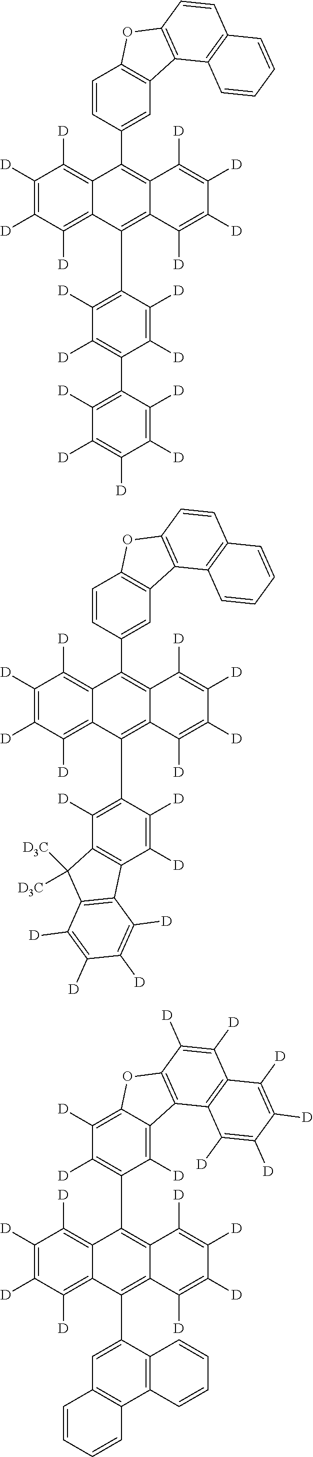

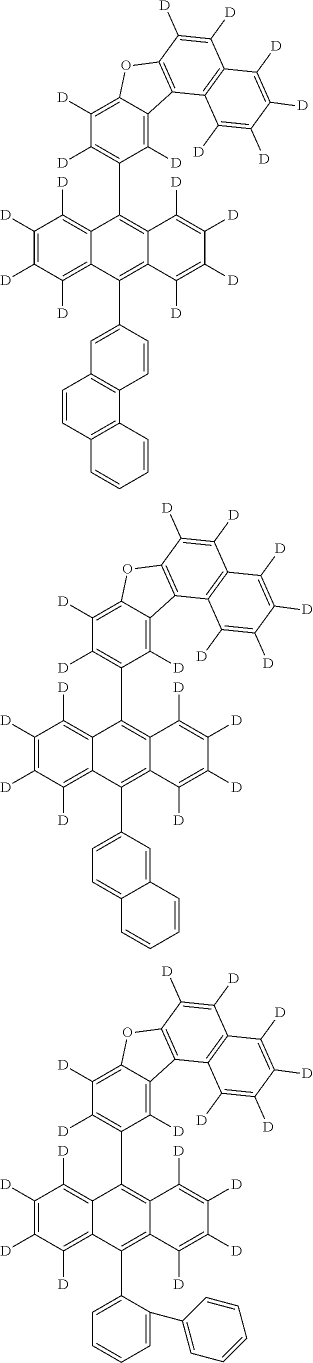

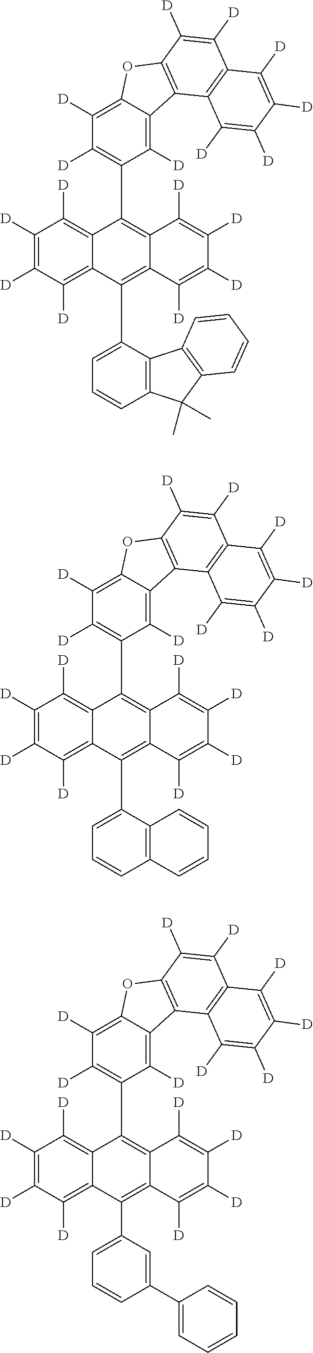

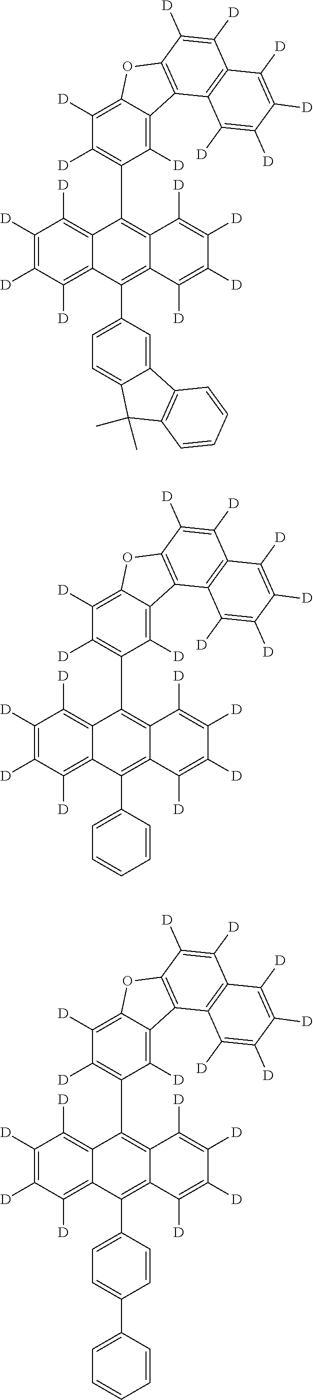

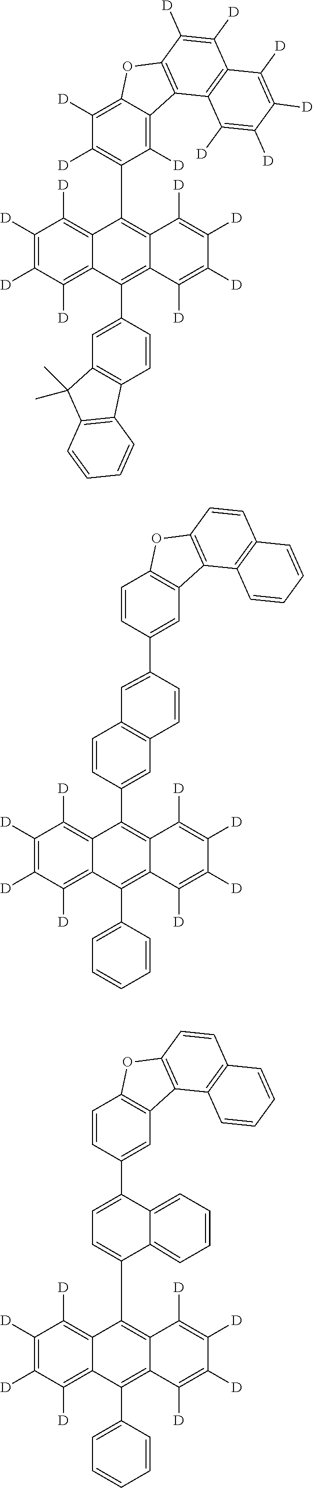 [SMILES: [2H]c1c(-c2c3c([2H])c([2H])c([2H])c([2H])c3c(-c3ccc4c(c3)C(C)(C)c3ccccc3-4)c3c([2H])c([2H])c([2H])c([2H])c23)c([2H])c2c(oc3c([2H])c([2H])c4c([2H])c([2H])c([2H])c([2H])c4c32)c1[2H] ; [2H]c1c([2H])c([2H])c2c(-c3ccc4cc(-c5ccc6oc7ccc8ccccc8c7c6c5)ccc4c3)c3c([2H])c([2H])c([2H])c([2H])c3c(-c3ccccc3)c2c1[2H] ; [2H]c1c([2H])c([2H])c2c(-c3ccc(-c4ccc5oc6ccc7ccccc7c6c5c4)c4ccccc34)c3c([2H])c([2H])c([2H])c([2H])c3c(-c3ccccc3)c2c1[2H]]

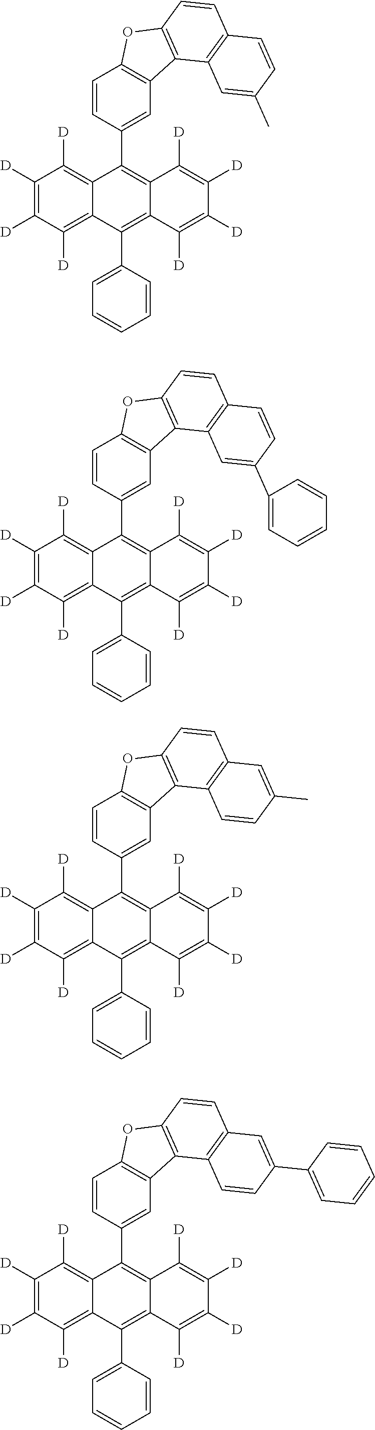 [SMILES: [2H]c1c([2H])c([2H])c2c(-c3ccc4oc5ccc6ccc(C)cc6c5c4c3)c3c([2H])c([2H])c([2H])c([2H])c3c(-c3ccccc3)c2c1[2H] ; [2H]c1c([2H])c([2H])c2c(-c3ccc4oc5ccc6ccc(-c7ccccc7)cc6c5c4c3)c3c([2H])c([2H])c([2H])c([2H])c3c(-c3ccccc3)c2c1[2H] ; [2H]c1c([2H])c([2H])c2c(-c3ccc4oc5ccc6cc(C)ccc6c5c4c3)c3c([2H])c([2H])c([2H])c([2H])c3c(-c3ccccc3)c2c1[2H] ; [2H]c1c([2H])c([2H])c2c(-c3ccc4oc5ccc6cc(-c7ccccc7)ccc6c5c4c3)c3c([2H])c([2H])c([2H])c([2H])c3c(-c3ccccc3)c2c1[2H]]

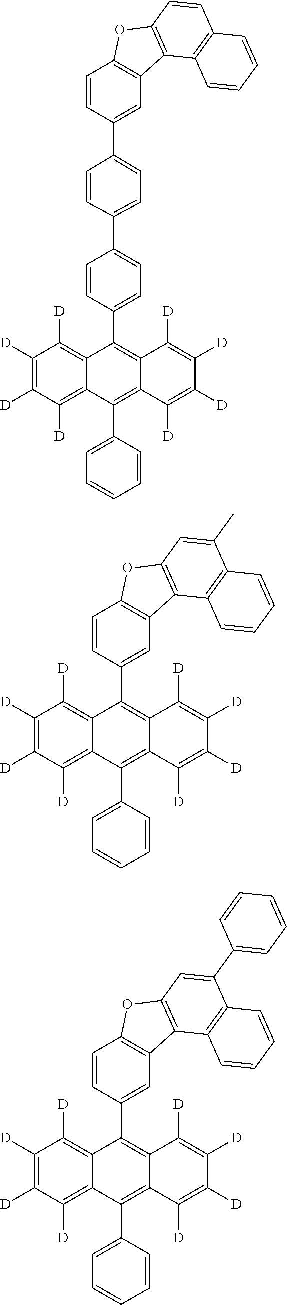 [SMILES: [2H]c1c([2H])c([2H])c2c(-c3ccc(-c4ccc(-c5ccc6oc7ccc8ccccc8c7c6c5)cc4)cc3)c3c([2H])c([2H])c([2H])c([2H])c3c(-c3ccccc3)c2c1[2H] ; [2H]c1c([2H])c([2H])c2c(-c3ccc4oc5cc(C)c6ccccc6c5c4c3)c3c([2H])c([2H])c([2H])c([2H])c3c(-c3ccccc3)c2c1[2H] ; [2H]c1c([2H])c([2H])c2c(-c3ccc4oc5cc(-c6ccccc6)c6ccccc6c5c4c3)c3c([2H])c([2H])c([2H])c([2H])c3c(-c3ccccc3)c2c1[2H]]

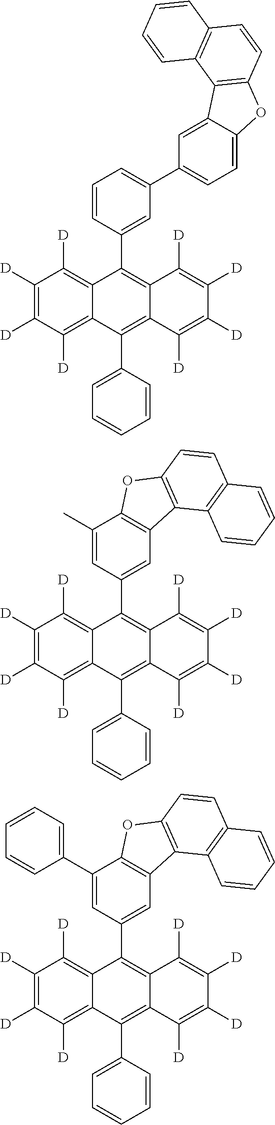

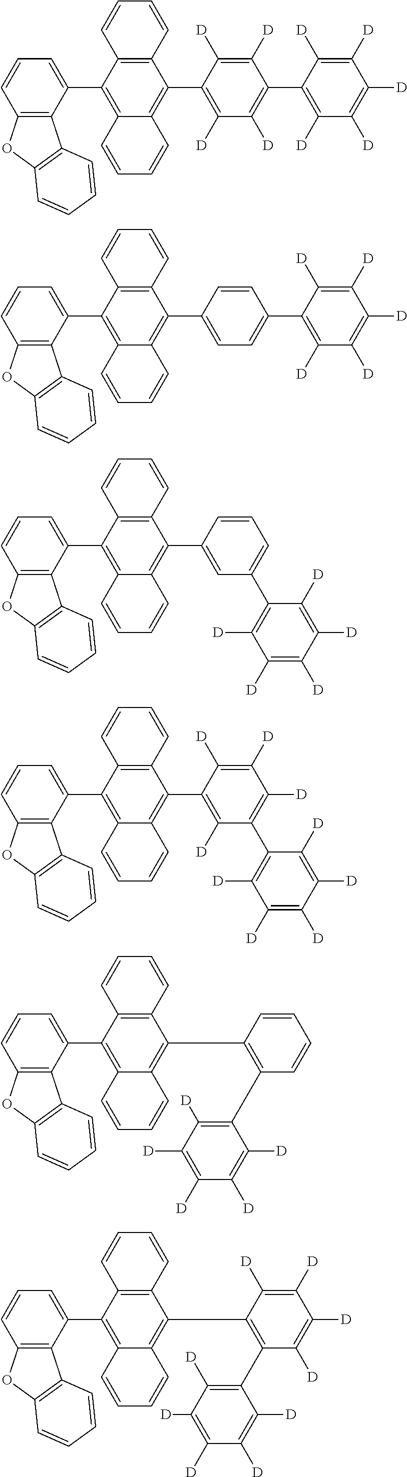

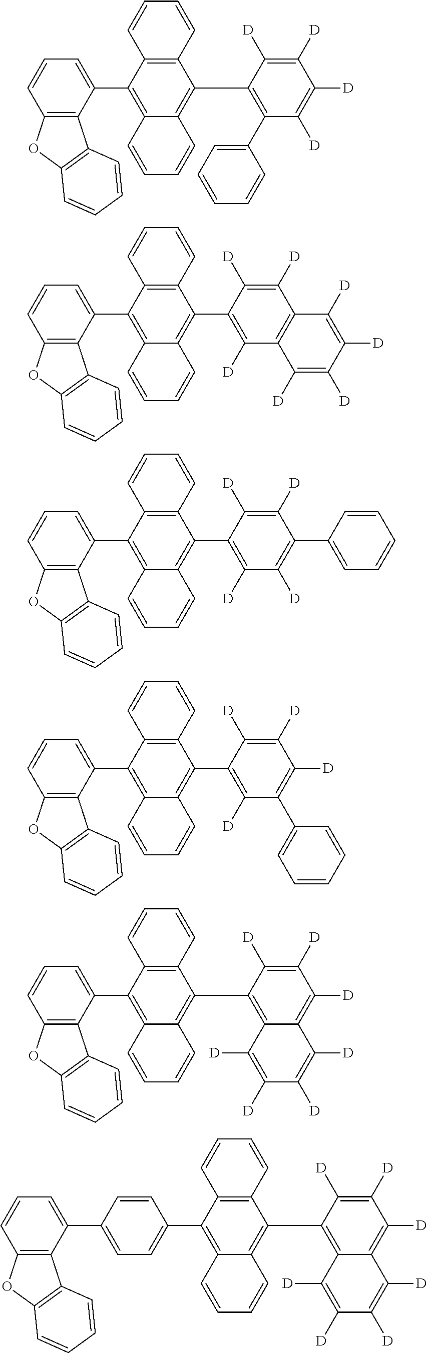

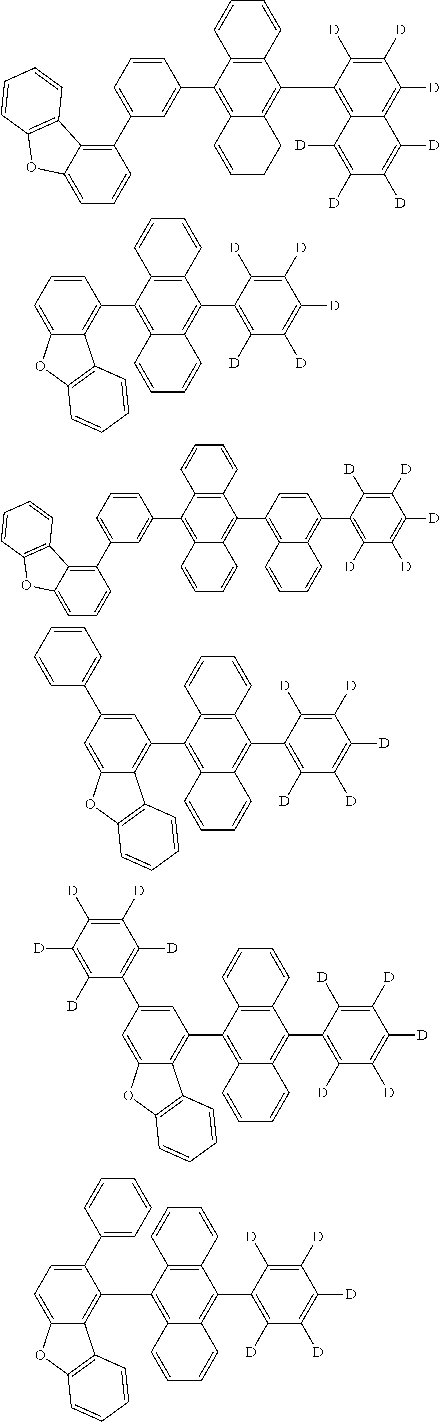

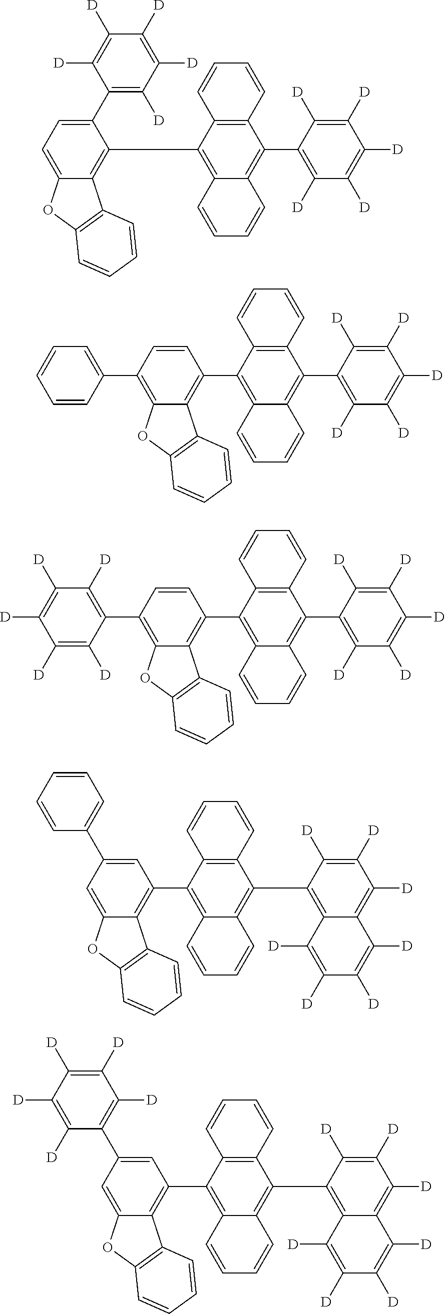

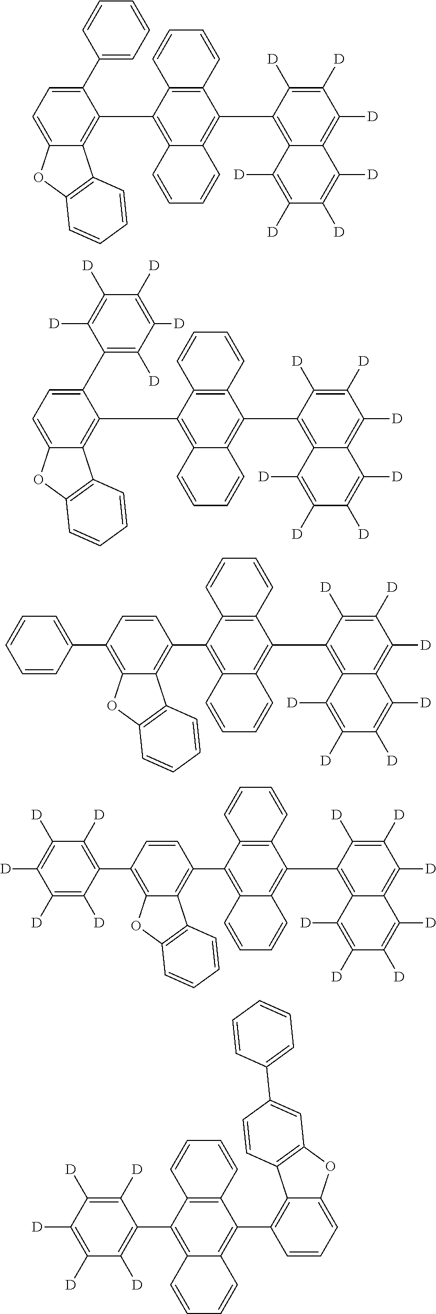

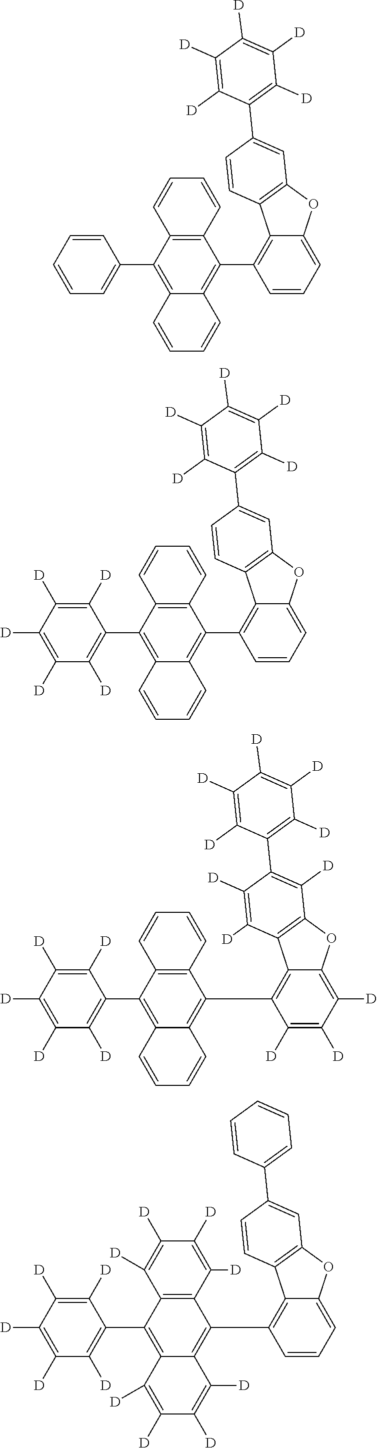

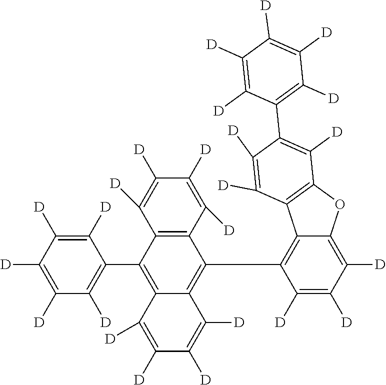 [SMILES: [2H]c1c([2H])c([2H])c(-c2c([2H])c([2H])c3c(oc4c([2H])c([2H])c([2H])c(-c5c6c([2H])c([2H])c([2H])c([2H])c6c(-c6c([2H])c([2H])c([2H])c([2H])c6[2H])c6c([2H])c([2H])c([2H])c([2H])c56)c43)c2[2H])c([2H])c1[2H]]

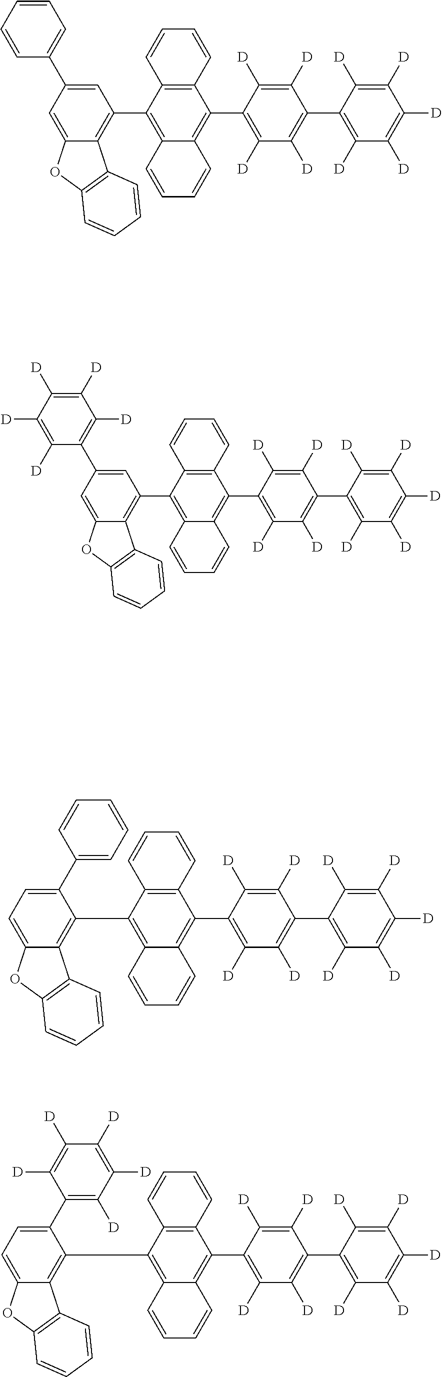

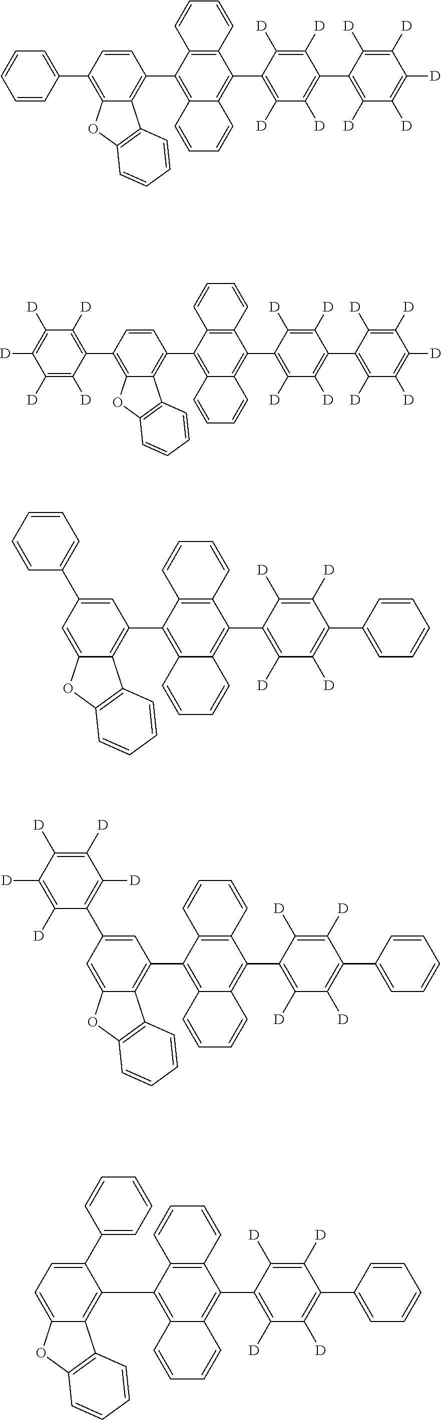

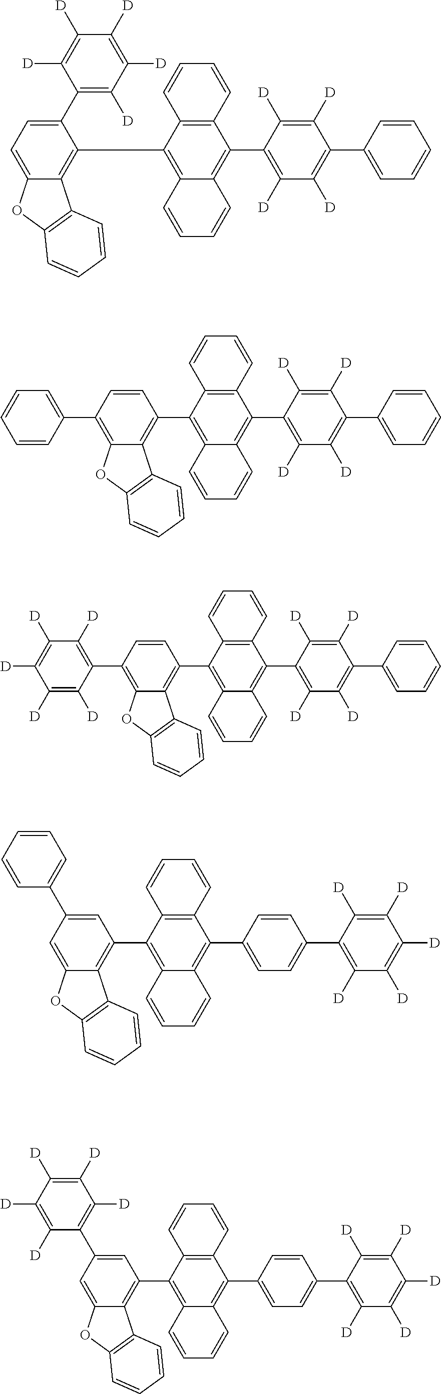

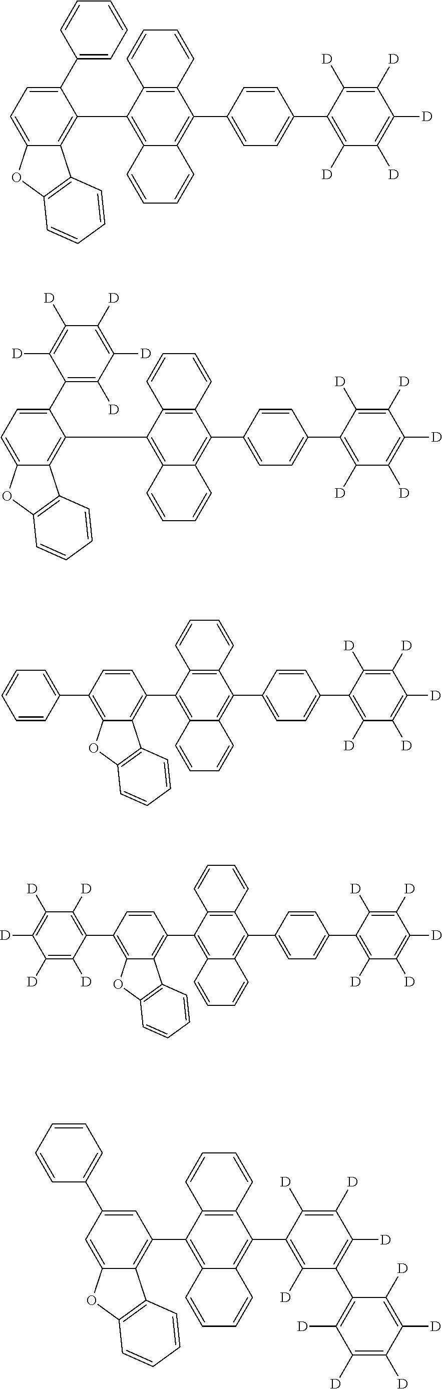

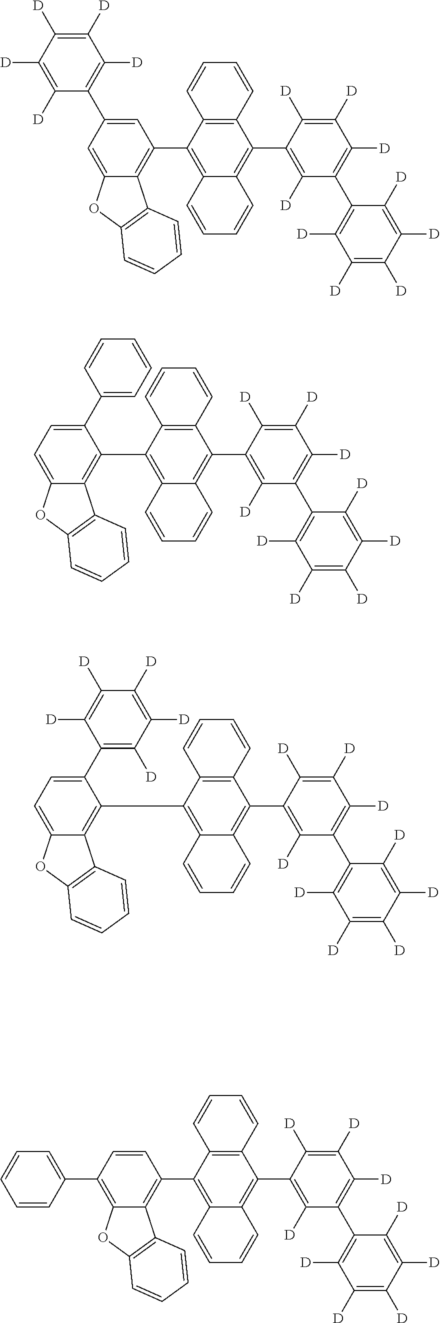

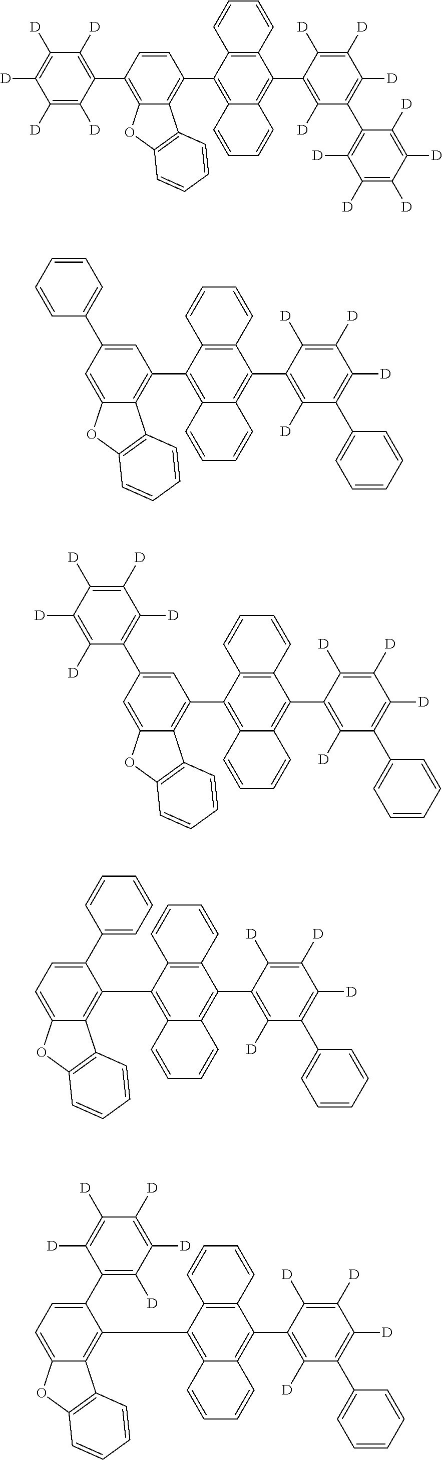

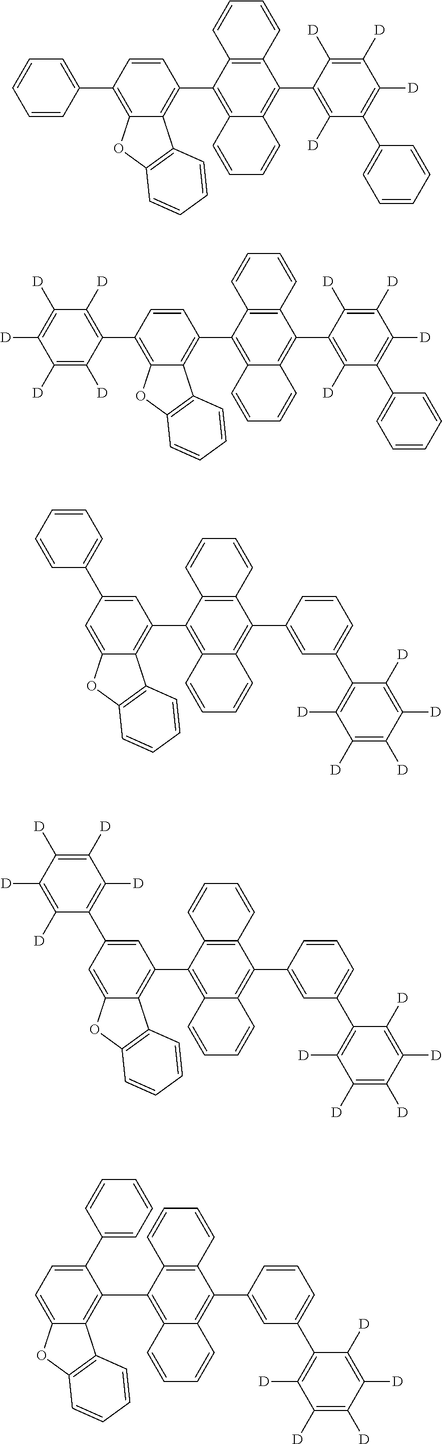

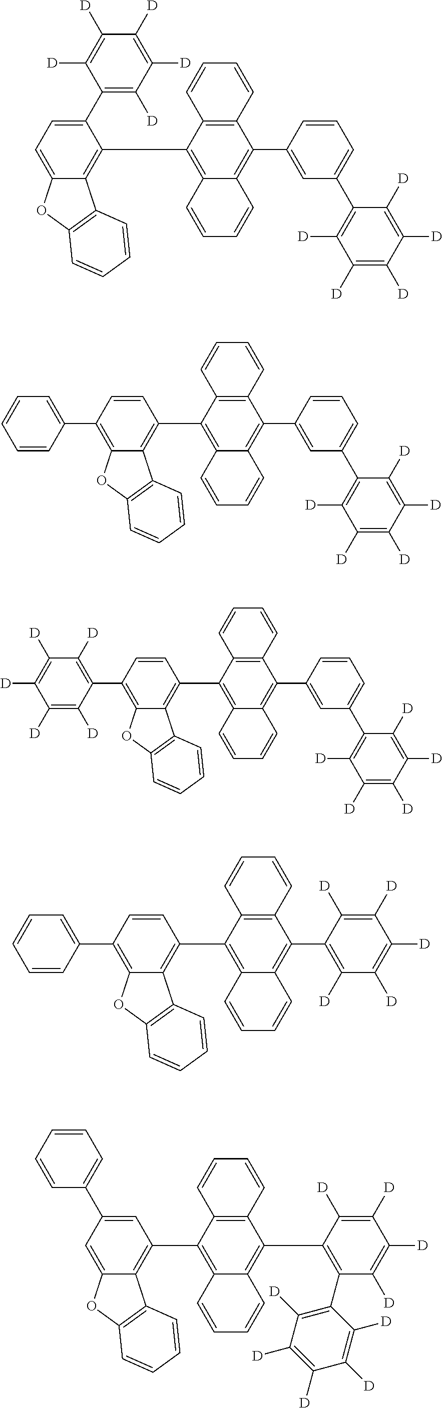

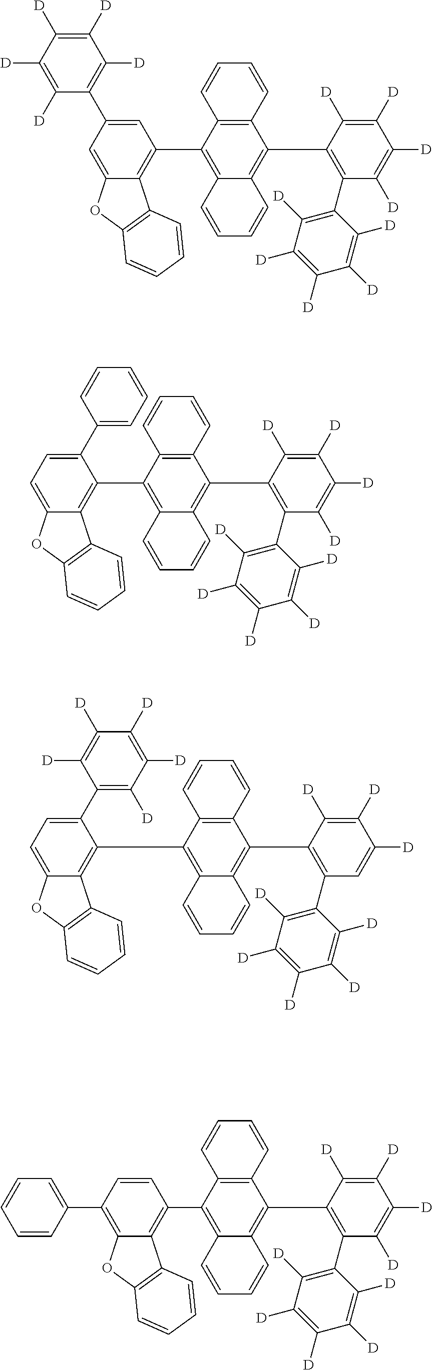

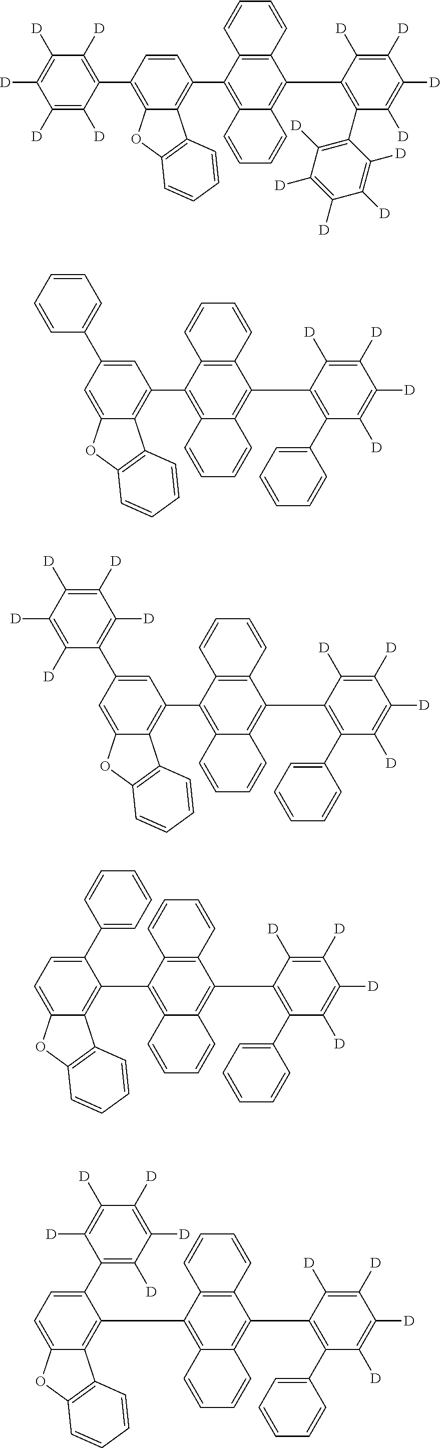

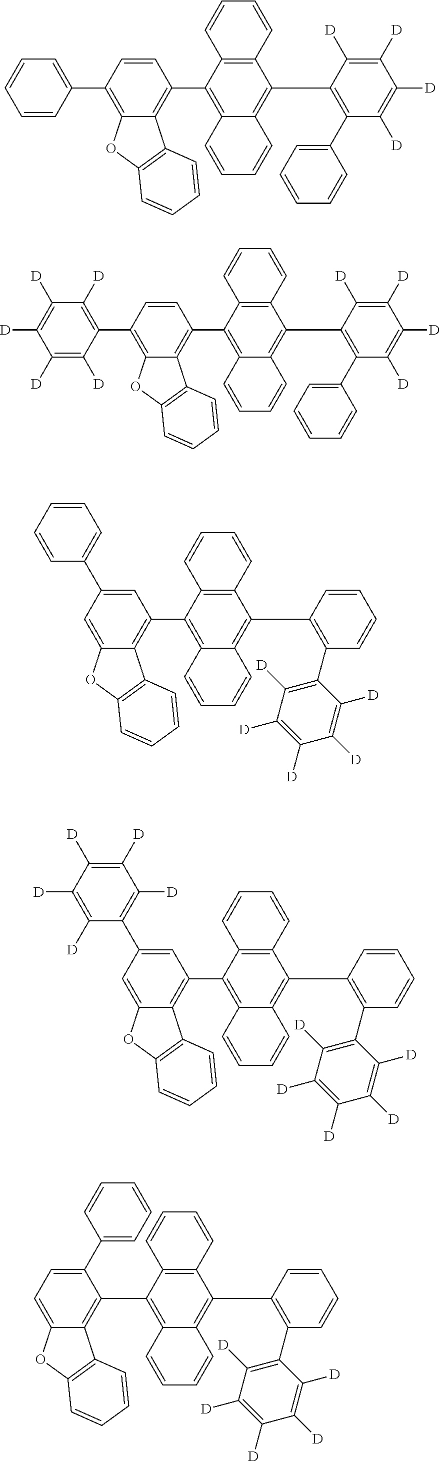

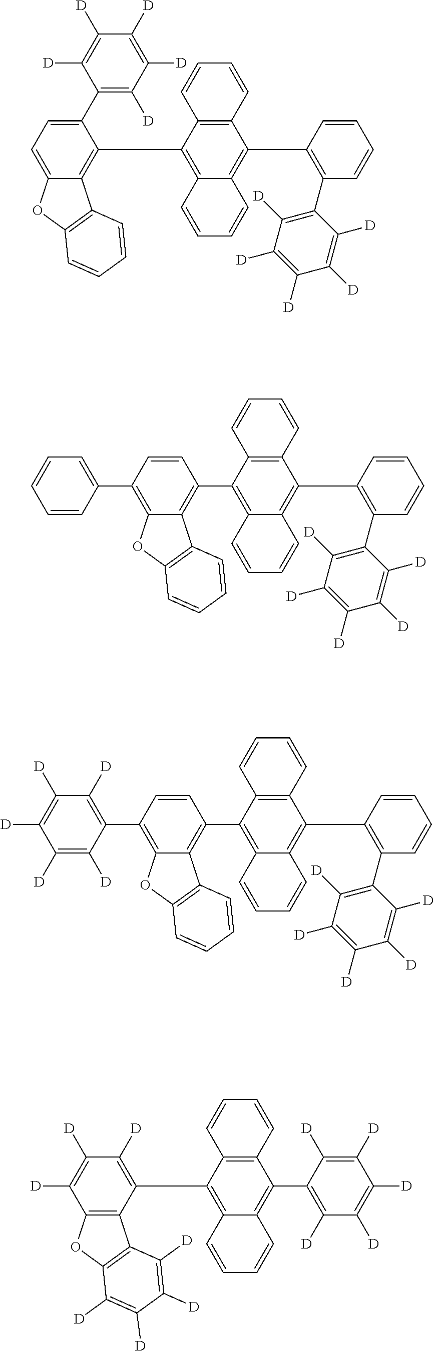 [SMILES: [2H]c1c([2H])c([2H])c(-c2ccccc2-c2c3ccccc3c(-c3c(-c4c([2H])c([2H])c([2H])c([2H])c4[2H])ccc4oc5ccccc5c34)c3ccccc23)c([2H])c1[2H] ; [2H]c1c([2H])c([2H])c(-c2ccccc2-c2c3ccccc3c(-c3ccc(-c4ccccc4)c4oc5ccccc5c34)c3ccccc23)c([2H])c1[2H] ; [2H]c1c([2H])c([2H])c(-c2ccccc2-c2c3ccccc3c(-c3ccc(-c4c([2H])c([2H])c([2H])c([2H])c4[2H])c4oc5ccccc5c34)c3ccccc23)c([2H])c1[2H] ; [2H]c1c([2H])c([2H])c(-c2c3ccccc3c(-c3c([2H])c([2H])c([2H])c4oc5c([2H])c([2H])c([2H])c([2H])c5c34)c3ccccc23)c([2H])c1[2H]]

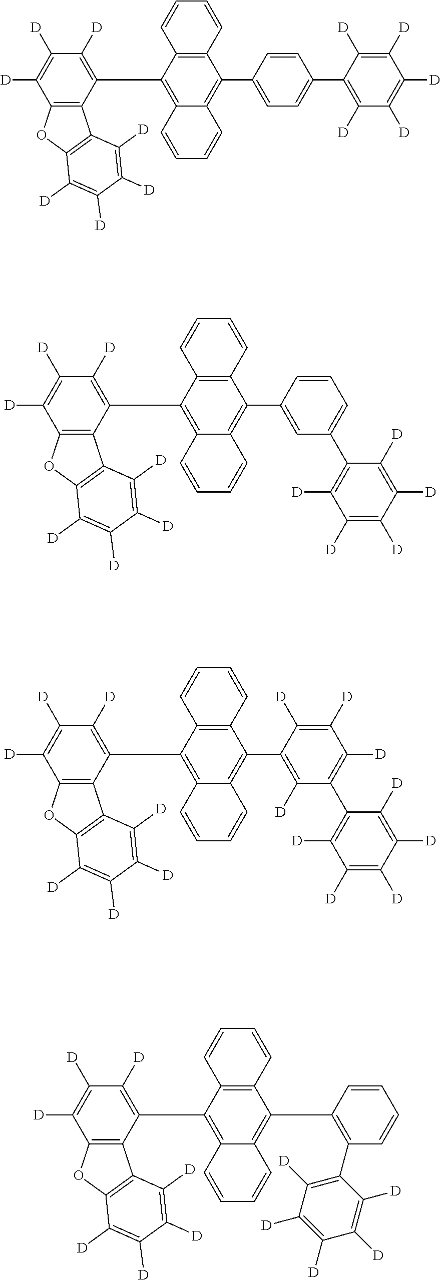

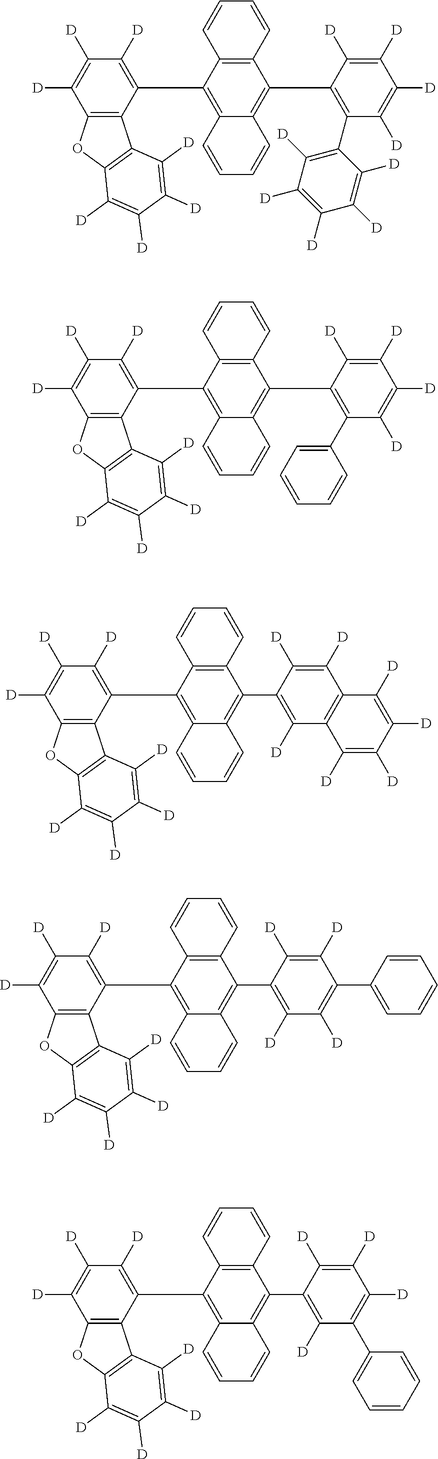

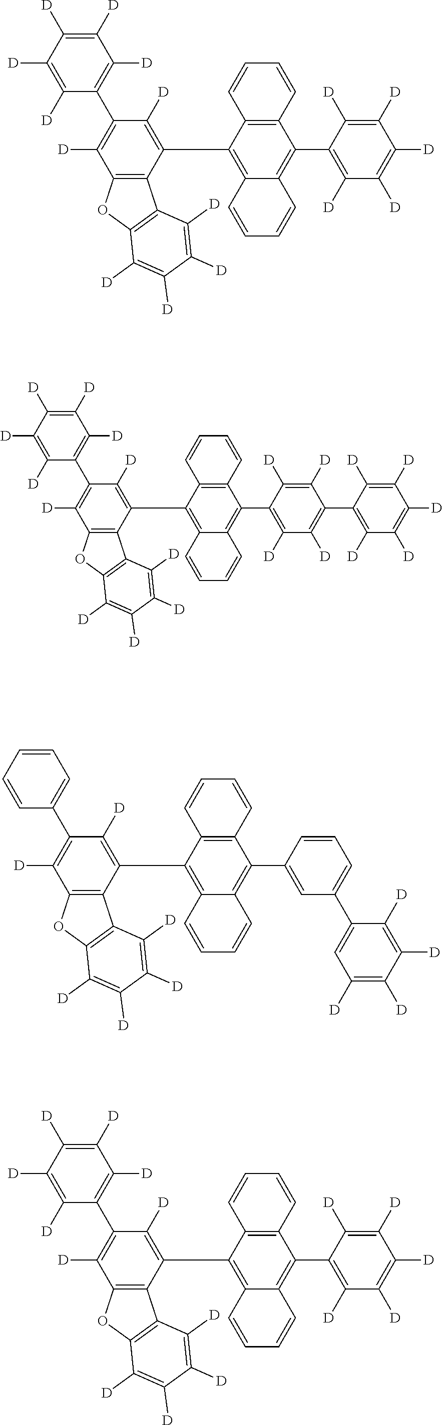

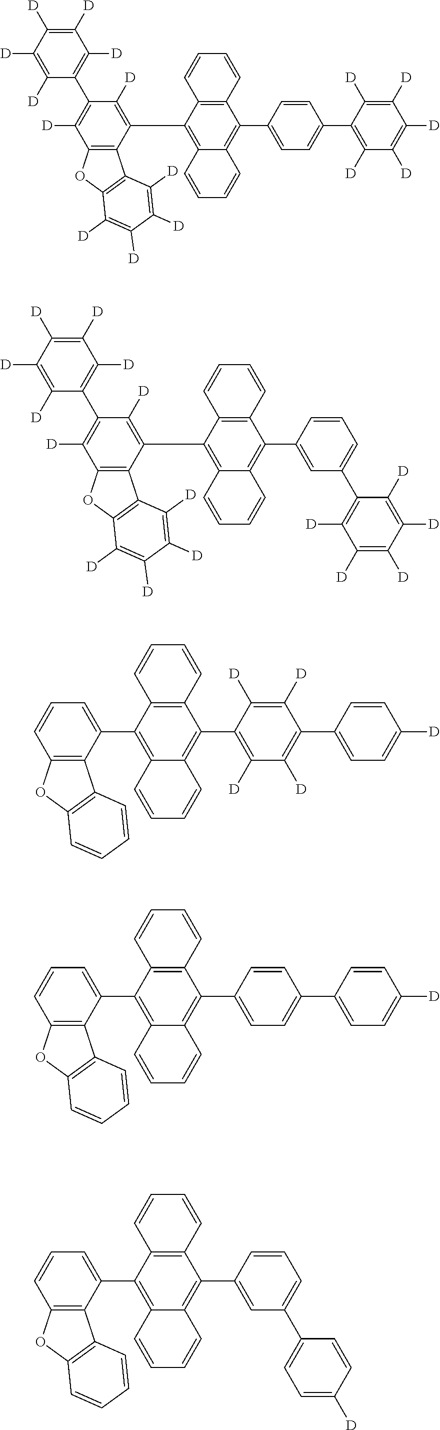

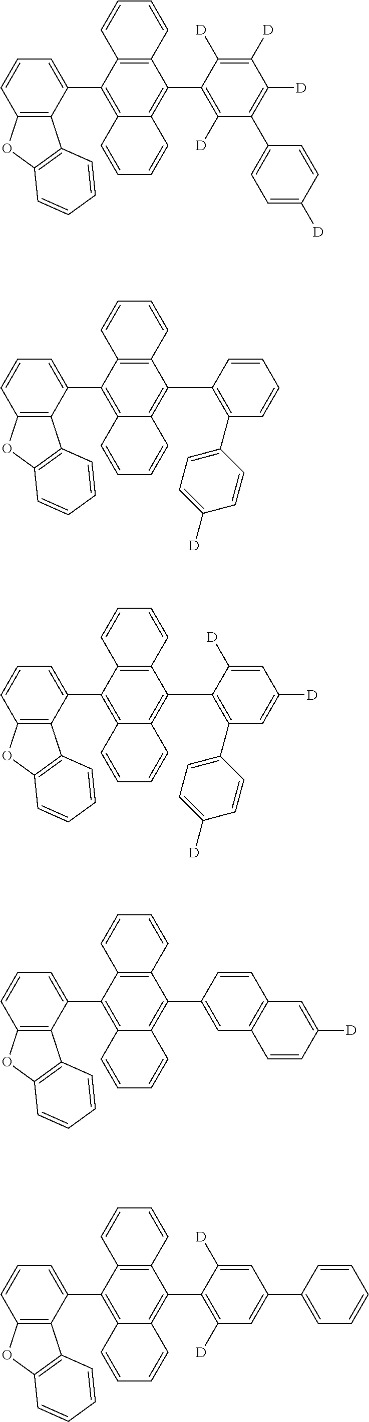 [SMILES: [2H]c1ccc(-c2c([2H])c([2H])c([2H])c(-c3c4ccccc4c(-c4cccc5oc6ccccc6c45)c4ccccc34)c2[2H])cc1 ; [2H]c1ccc(-c2ccccc2-c2c3ccccc3c(-c3cccc4oc5ccccc5c34)c3ccccc23)cc1 ; [2H]c1ccc(-c2cc([2H])cc([2H])c2-c2c3ccccc3c(-c3cccc4oc5ccccc5c34)c3ccccc23)cc1 ; [2H]c1ccc2cc(-c3c4ccccc4c(-c4cccc5oc6ccccc6c45)c4ccccc34)ccc2c1 ; [2H]c1cc(-c2ccccc2)cc([2H])c1-c1c2ccccc2c(-c2cccc3oc4ccccc4c23)c2ccccc12]

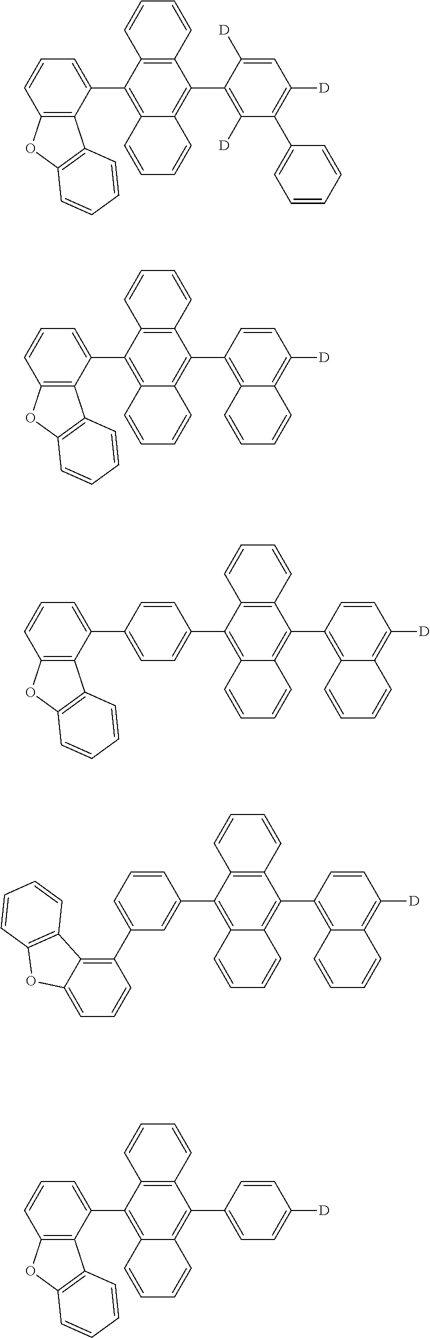 [SMILES: [2H]c1cc([2H])c(-c2c3ccccc3c(-c3cccc4oc5ccccc5c34)c3ccccc23)c([2H])c1-c1ccccc1 ; [2H]c1ccc(-c2c3ccccc3c(-c3cccc4oc5ccccc5c34)c3ccccc23)c2ccccc12 ; [2H]c1ccc(-c2c3ccccc3c(-c3ccc(-c4cccc5oc6ccccc6c45)cc3)c3ccccc23)c2ccccc12 ; [2H]c1ccc(-c2c3ccccc3c(-c3cccc(-c4cccc5oc6ccccc6c45)c3)c3ccccc23)c2ccccc12 ; [2H]c1ccc(-c2c3ccccc3c(-c3cccc4oc5ccccc5c34)c3ccccc23)cc1]

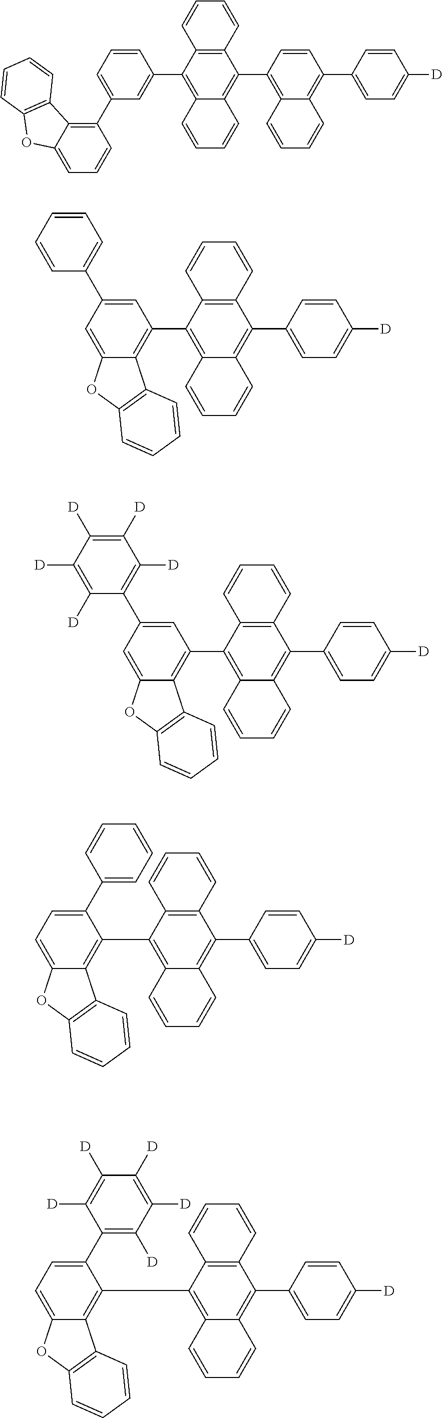

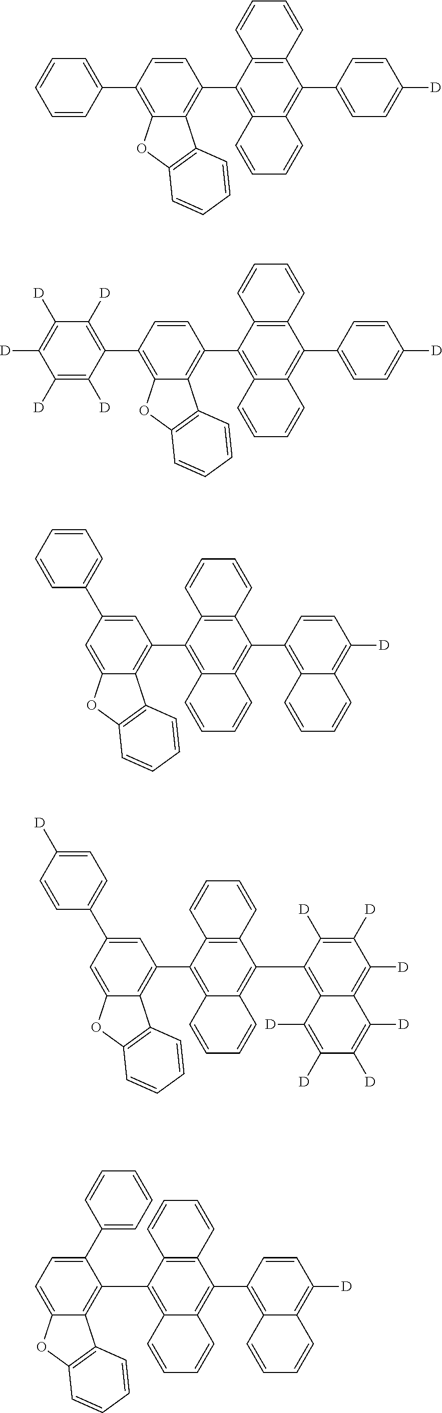 [SMILES: [2H]c1ccc(-c2c3ccccc3c(-c3ccc(-c4ccccc4)c4oc5ccccc5c34)c3ccccc23)cc1 ; [2H]c1ccc(-c2c3ccccc3c(-c3ccc(-c4c([2H])c([2H])c([2H])c([2H])c4[2H])c4oc5ccccc5c34)c3ccccc23)cc1 ; [2H]c1ccc(-c2c3ccccc3c(-c3cc(-c4ccccc4)cc4oc5ccccc5c34)c3ccccc23)c2ccccc12 ; [2H]c1ccc(-c2cc(-c3c4ccccc4c(-c4c([2H])c([2H])c([2H])c5c([2H])c([2H])c([2H])c([2H])c45)c4ccccc34)c3c(c2)oc2ccccc23)cc1 ; [2H]c1ccc(-c2c3ccccc3c(-c3c(-c4ccccc4)ccc4oc5ccccc5c34)c3ccccc23)c2ccccc12]

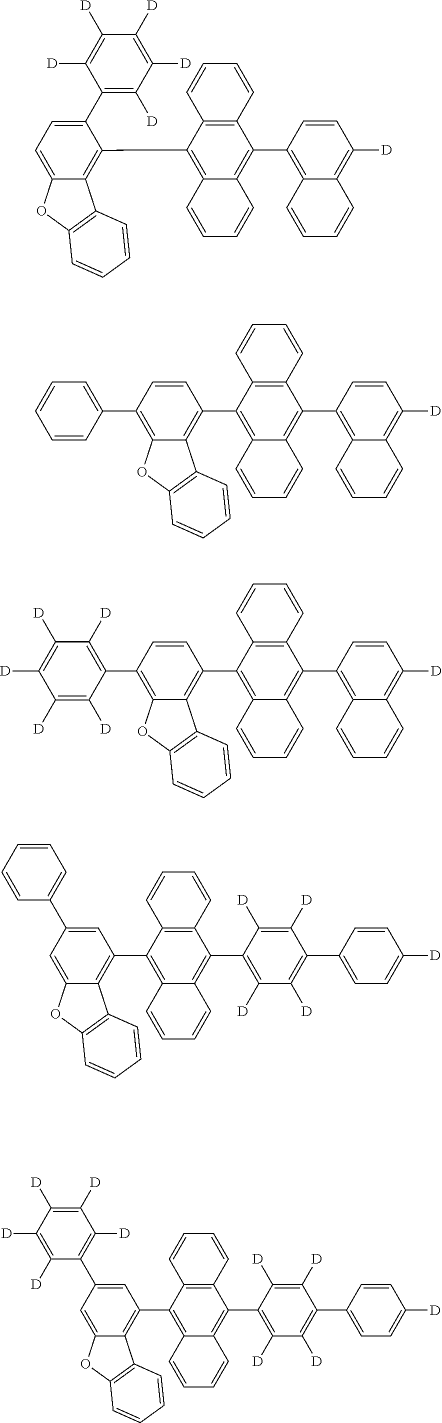

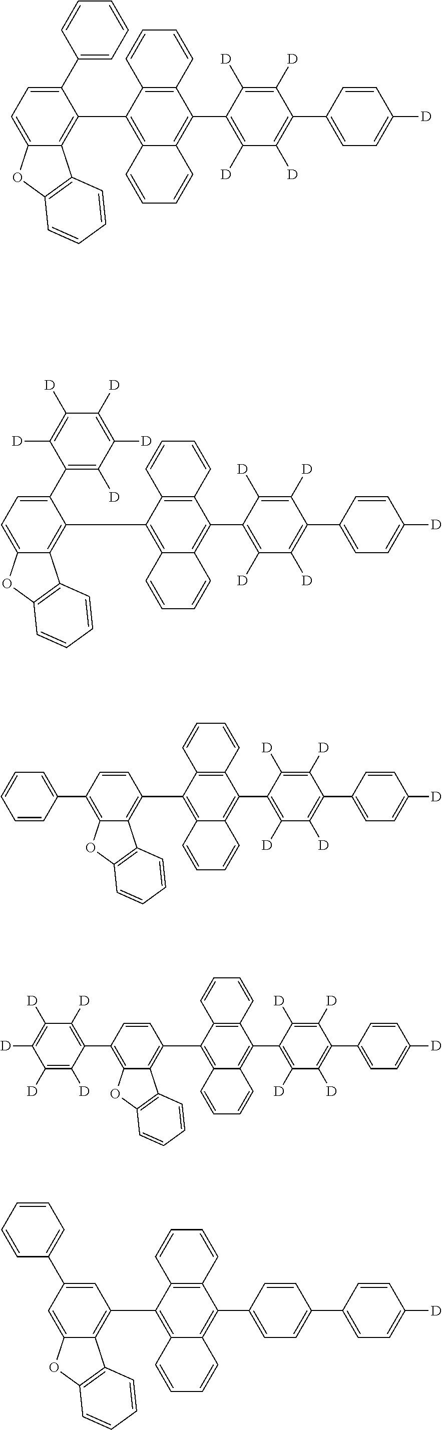

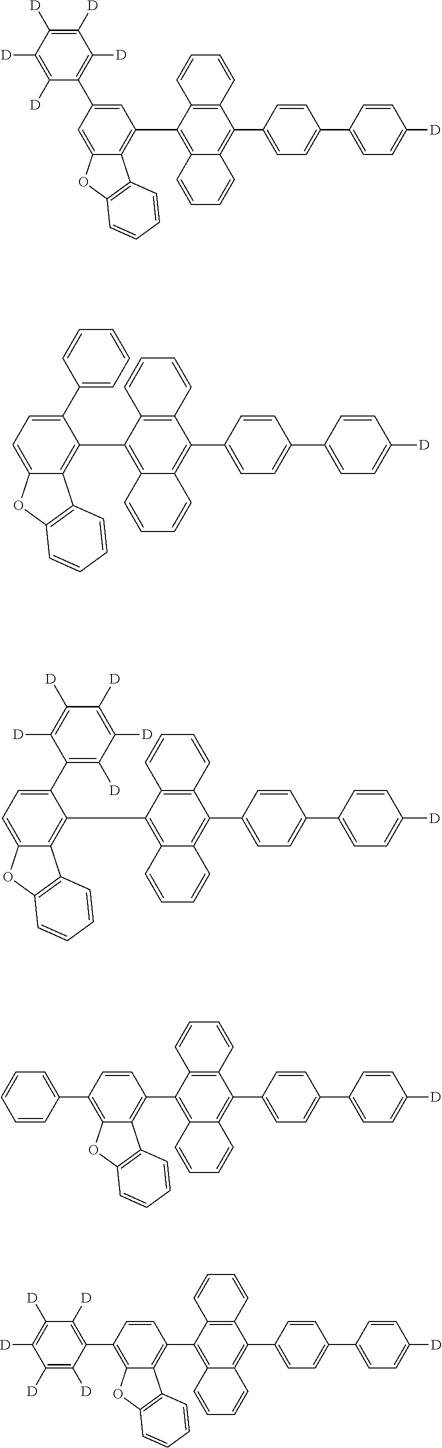 [SMILES: [2H]c1ccc(-c2ccc(-c3c4ccccc4c(-c4cc(-c5c([2H])c([2H])c([2H])c([2H])c5[2H])cc5oc6ccccc6c45)c4ccccc34)cc2)cc1 ; [2H]c1ccc(-c2ccc(-c3c4ccccc4c(-c4c(-c5ccccc5)ccc5oc6ccccc6c45)c4ccccc34)cc2)cc1 ; [2H]c1ccc(-c2ccc(-c3c4ccccc4c(-c4c(-c5c([2H])c([2H])c([2H])c([2H])c5[2H])ccc5oc6ccccc6c45)c4ccccc34)cc2)cc1 ; [2H]c1ccc(-c2ccc(-c3c4ccccc4c(-c4ccc(-c5ccccc5)c5oc6ccccc6c45)c4ccccc34)cc2)cc1 ; [2H]c1ccc(-c2ccc(-c3c4ccccc4c(-c4ccc(-c5c([2H])c([2H])c([2H])c([2H])c5[2H])c5oc6ccccc6c45)c4ccccc34)cc2)cc1]

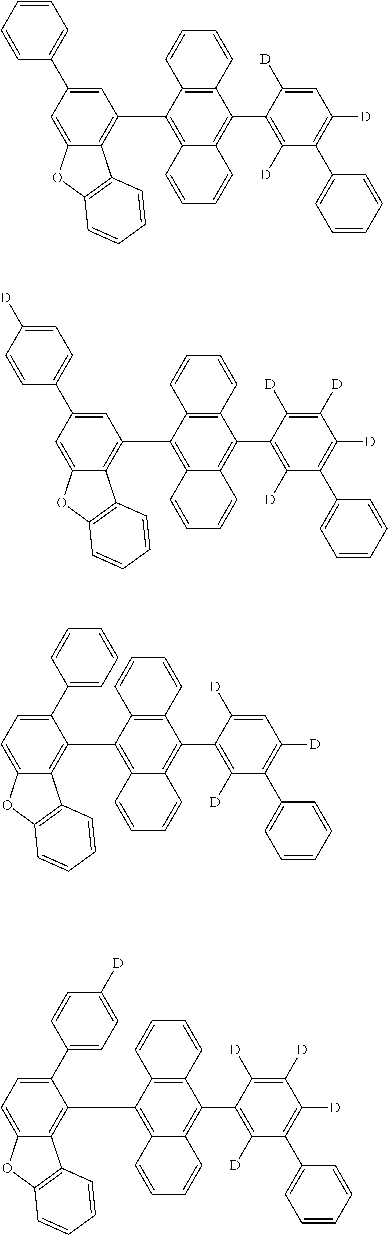 [SMILES: [2H]c1cc([2H])c(-c2c3ccccc3c(-c3cc(-c4ccccc4)cc4oc5ccccc5c34)c3ccccc23)c([2H])c1-c1ccccc1 ; [2H]c1ccc(-c2cc(-c3c4ccccc4c(-c4c([2H])c([2H])c([2H])c(-c5ccccc5)c4[2H])c4ccccc34)c3c(c2)oc2ccccc23)cc1 ; [2H]c1cc([2H])c(-c2c3ccccc3c(-c3c(-c4ccccc4)ccc4oc5ccccc5c34)c3ccccc23)c([2H])c1-c1ccccc1 ; [2H]c1ccc(-c2ccc3oc4ccccc4c3c2-c2c3ccccc3c(-c3c([2H])c([2H])c([2H])c(-c4ccccc4)c3[2H])c3ccccc23)cc1]

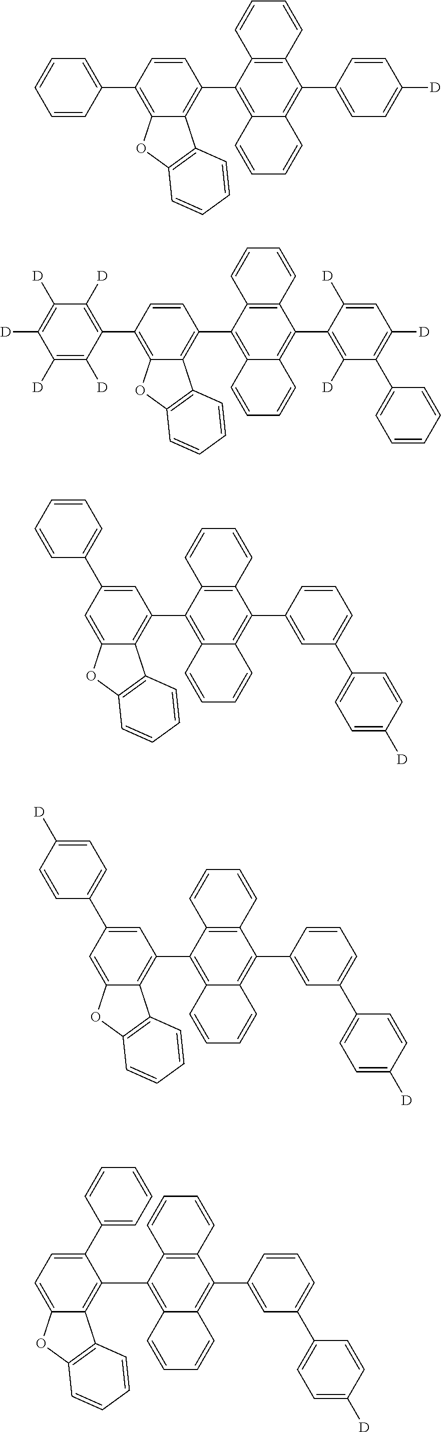 [SMILES: [2H]c1ccc(-c2c3ccccc3c(-c3ccc(-c4ccccc4)c4oc5ccccc5c34)c3ccccc23)cc1 ; [2H]c1cc([2H])c(-c2c3ccccc3c(-c3ccc(-c4c([2H])c([2H])c([2H])c([2H])c4[2H])c4oc5ccccc5c34)c3ccccc23)c([2H])c1-c1ccccc1 ; [2H]c1ccc(-c2cccc(-c3c4ccccc4c(-c4cc(-c5ccccc5)cc5oc6ccccc6c45)c4ccccc34)c2)cc1 ; [2H]c1ccc(-c2cccc(-c3c4ccccc4c(-c4cc(-c5ccc([2H])cc5)cc5oc6ccccc6c45)c4ccccc34)c2)cc1 ; [2H]c1ccc(-c2cccc(-c3c4ccccc4c(-c4c(-c5ccccc5)ccc5oc6ccccc6c45)c4ccccc34)c2)cc1]

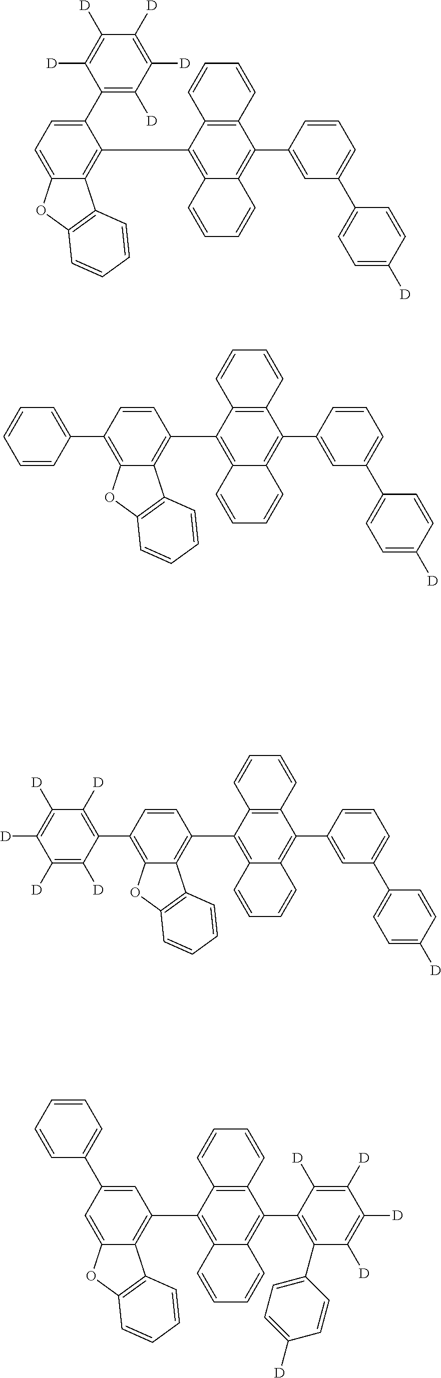 [SMILES: [2H]c1ccc(-c2cccc(-c3c4ccccc4c(-c4c(-c5c([2H])c([2H])c([2H])c([2H])c5[2H])ccc5oc6ccccc6c45)c4ccccc34)c2)cc1 ; [2H]c1ccc(-c2cccc(-c3c4ccccc4c(-c4ccc(-c5ccccc5)c5oc6ccccc6c45)c4ccccc34)c2)cc1 ; [2H]c1ccc(-c2cccc(-c3c4ccccc4c(-c4ccc(-c5c([2H])c([2H])c([2H])c([2H])c5[2H])c5oc6ccccc6c45)c4ccccc34)c2)cc1 ; [2H]c1ccc(-c2c([2H])c([2H])c([2H])c([2H])c2-c2c3ccccc3c(-c3cc(-c4ccccc4)cc4oc5ccccc5c34)c3ccccc23)cc1]

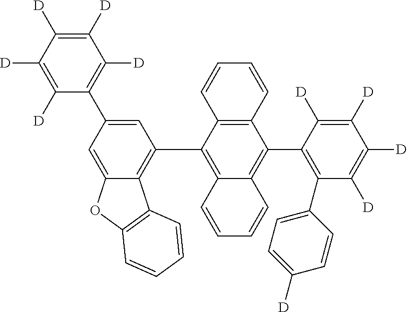 [SMILES: [2H]c1ccc(-c2c([2H])c([2H])c([2H])c([2H])c2-c2c3ccccc3c(-c3cc(-c4c([2H])c([2H])c([2H])c([2H])c4[2H])cc4oc5ccccc5c34)c3ccccc23)cc1]

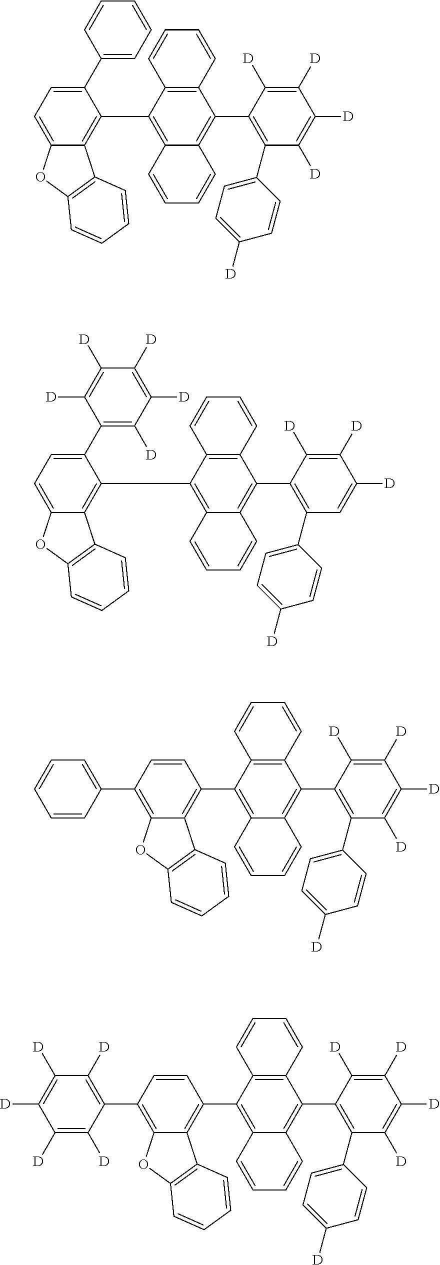 [SMILES: [2H]c1ccc(-c2c([2H])c([2H])c([2H])c([2H])c2-c2c3ccccc3c(-c3c(-c4ccccc4)ccc4oc5ccccc5c34)c3ccccc23)cc1 ; [2H]c1ccc(-c2cc([2H])c([2H])c([2H])c2-c2c3ccccc3c(-c3c(-c4c([2H])c([2H])c([2H])c([2H])c4[2H])ccc4oc5ccccc5c34)c3ccccc23)cc1 ; [2H]c1ccc(-c2c([2H])c([2H])c([2H])c([2H])c2-c2c3ccccc3c(-c3ccc(-c4ccccc4)c4oc5ccccc5c34)c3ccccc23)cc1 ; [2H]c1ccc(-c2c([2H])c([2H])c([2H])c([2H])c2-c2c3ccccc3c(-c3ccc(-c4c([2H])c([2H])c([2H])c([2H])c4[2H])c4oc5ccccc5c34)c3ccccc23)cc1]

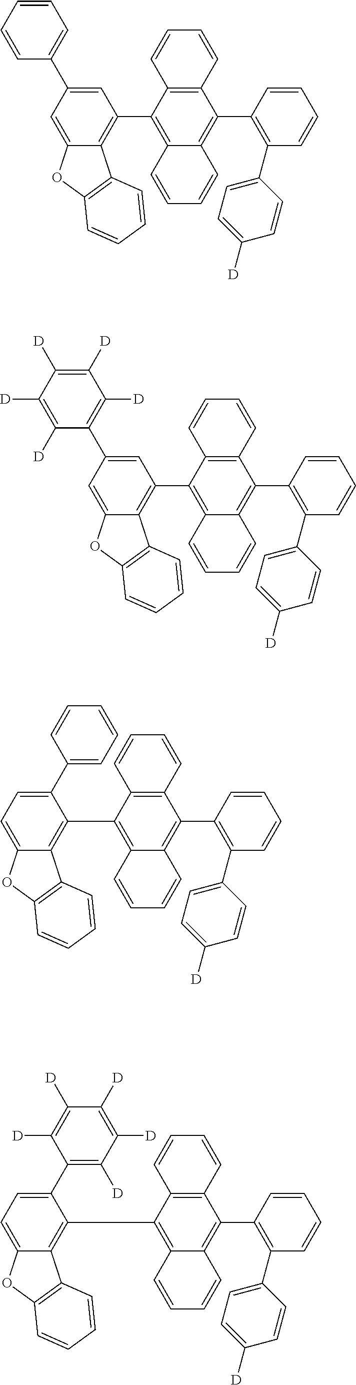 [SMILES: [2H]c1ccc(-c2ccccc2-c2c3ccccc3c(-c3cc(-c4ccccc4)cc4oc5ccccc5c34)c3ccccc23)cc1 ; [2H]c1ccc(-c2ccccc2-c2c3ccccc3c(-c3cc(-c4c([2H])c([2H])c([2H])c([2H])c4[2H])cc4oc5ccccc5c34)c3ccccc23)cc1 ; [2H]c1ccc(-c2ccccc2-c2c3ccccc3c(-c3c(-c4ccccc4)ccc4oc5ccccc5c34)c3ccccc23)cc1 ; [2H]c1ccc(-c2ccccc2-c2c3ccccc3c(-c3c(-c4c([2H])c([2H])c([2H])c([2H])c4[2H])ccc4oc5ccccc5c34)c3ccccc23)cc1]

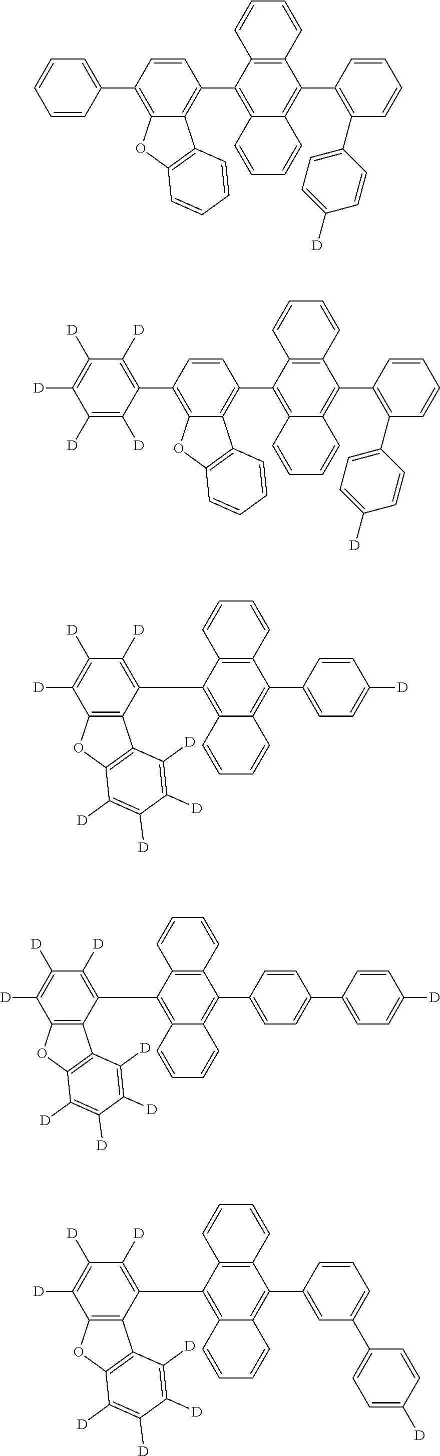

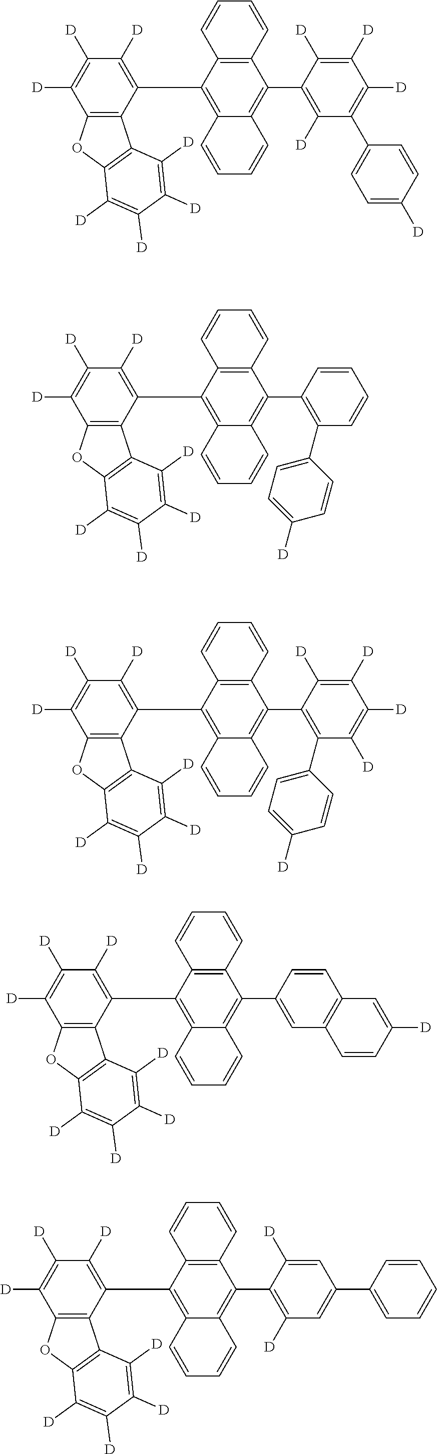

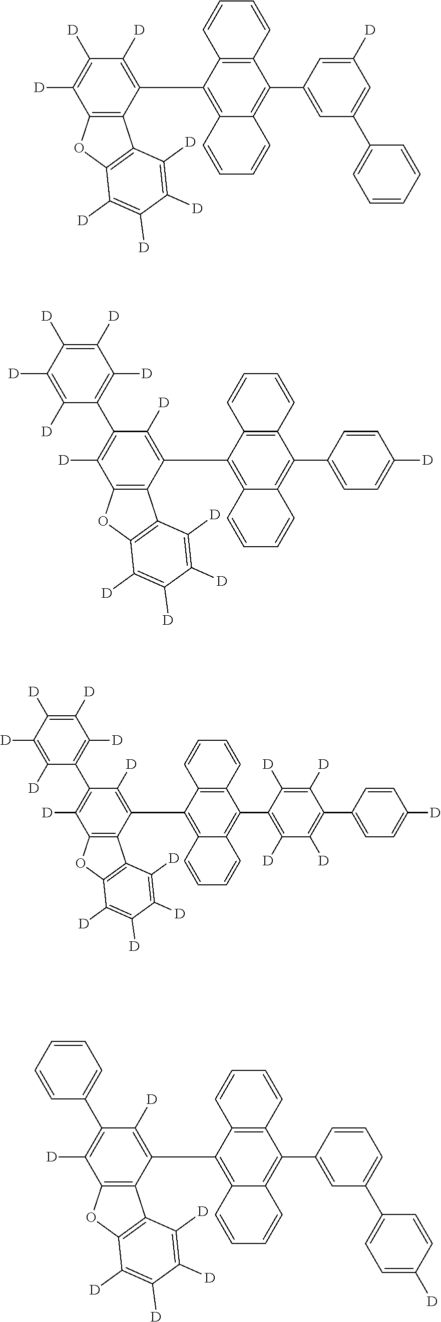

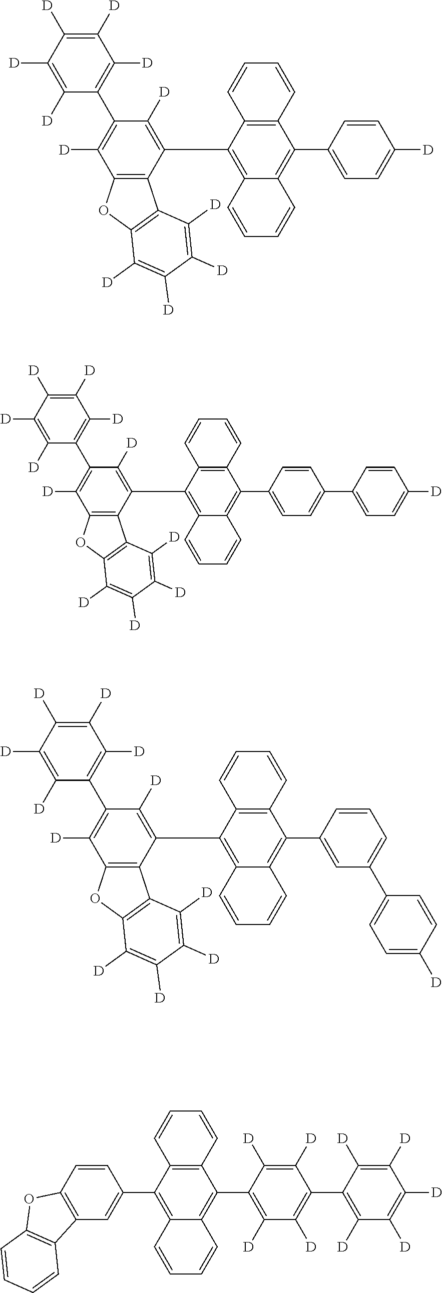

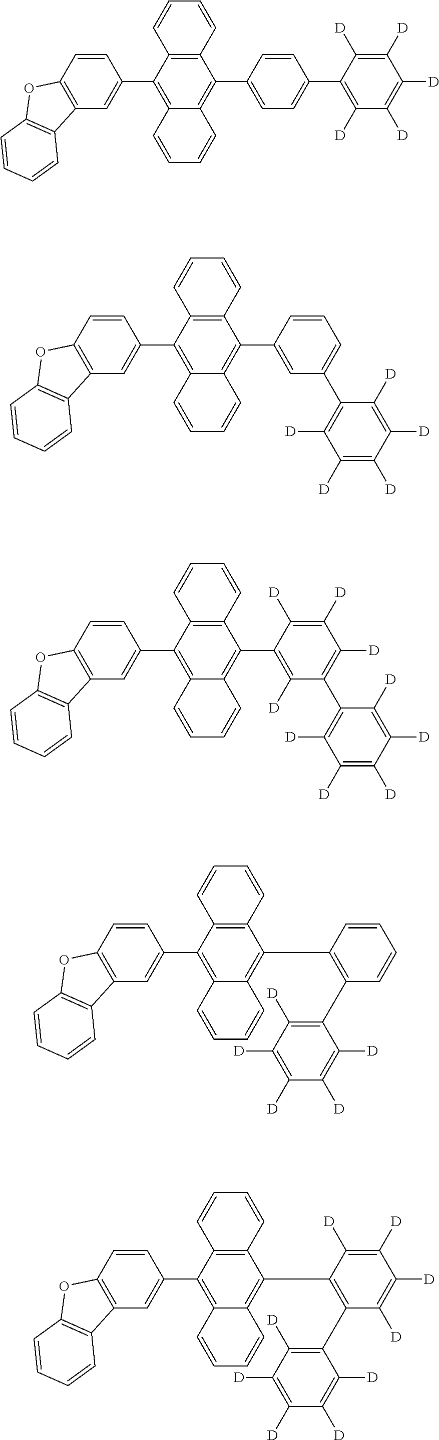

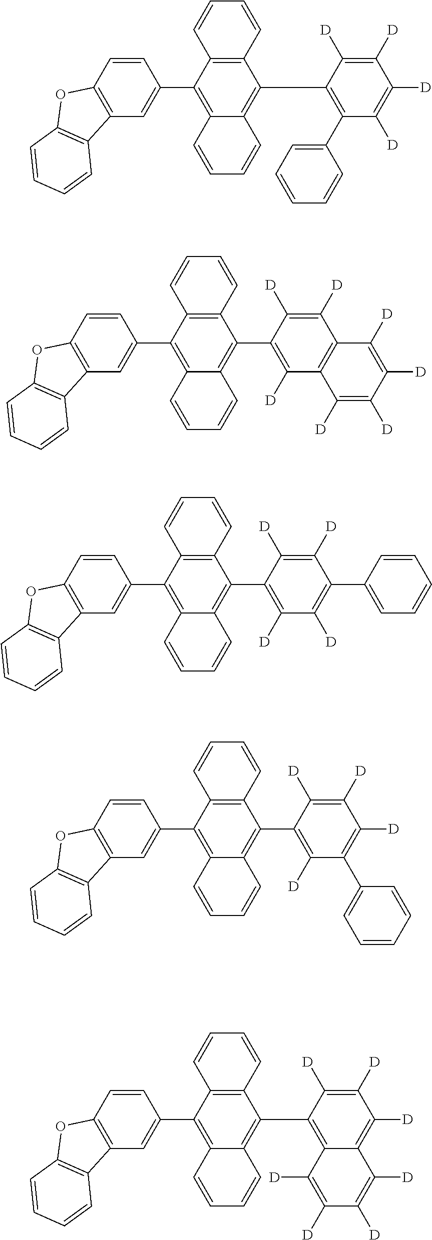 [SMILES: [2H]c1c([2H])c([2H])c(-c2c3ccccc3c(-c3ccc4oc5ccccc5c4c3)c3ccccc23)c(-c2ccccc2)c1[2H] ; [2H]c1c([2H])c([2H])c2c([2H])c(-c3c4ccccc4c(-c4ccc5oc6ccccc6c5c4)c4ccccc34)c([2H])c([2H])c2c1[2H] ; [2H]c1c([2H])c(-c2c3ccccc3c(-c3ccc4oc5ccccc5c4c3)c3ccccc23)c([2H])c([2H])c1-c1ccccc1 ; [2H]c1c([2H])c(-c2ccccc2)c([2H])c(-c2c3ccccc3c(-c3ccc4oc5ccccc5c4c3)c3ccccc23)c1[2H] ; [2H]c1c([2H])c([2H])c2c(-c3c4ccccc4c(-c4ccc5oc6ccccc6c5c4)c4ccccc34)c([2H])c([2H])c([2H])c2c1[2H]]

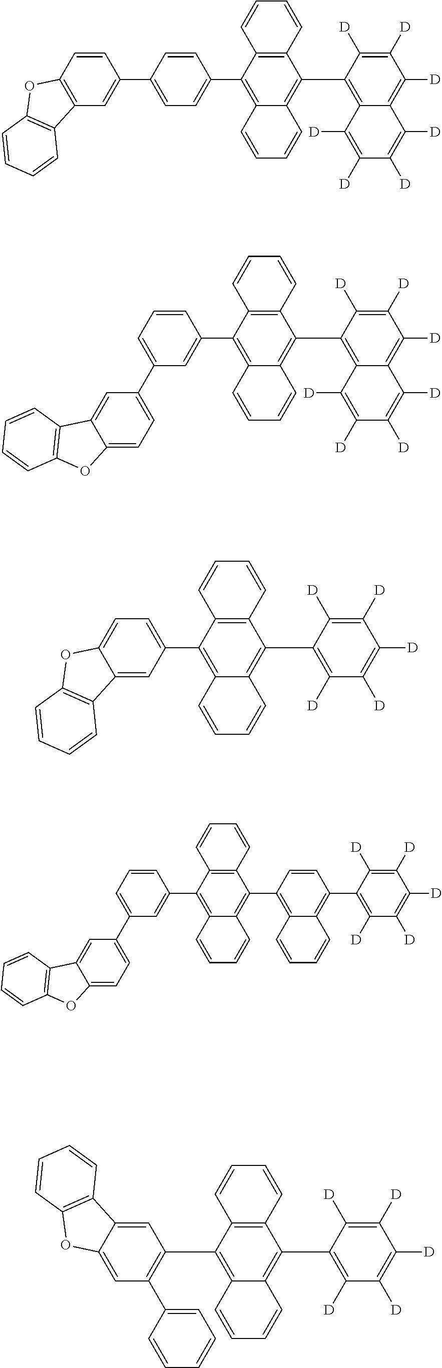

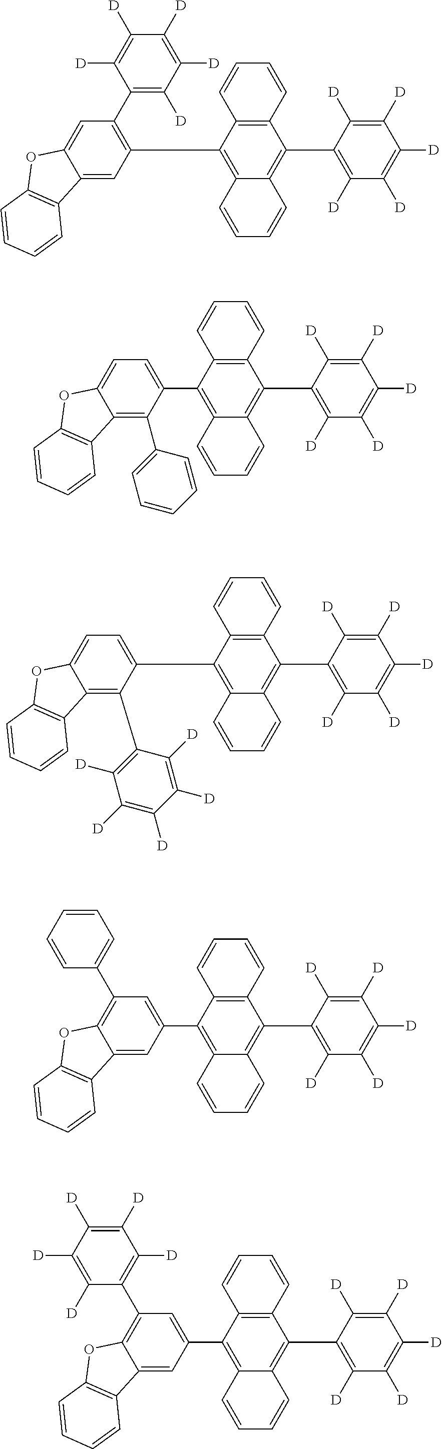

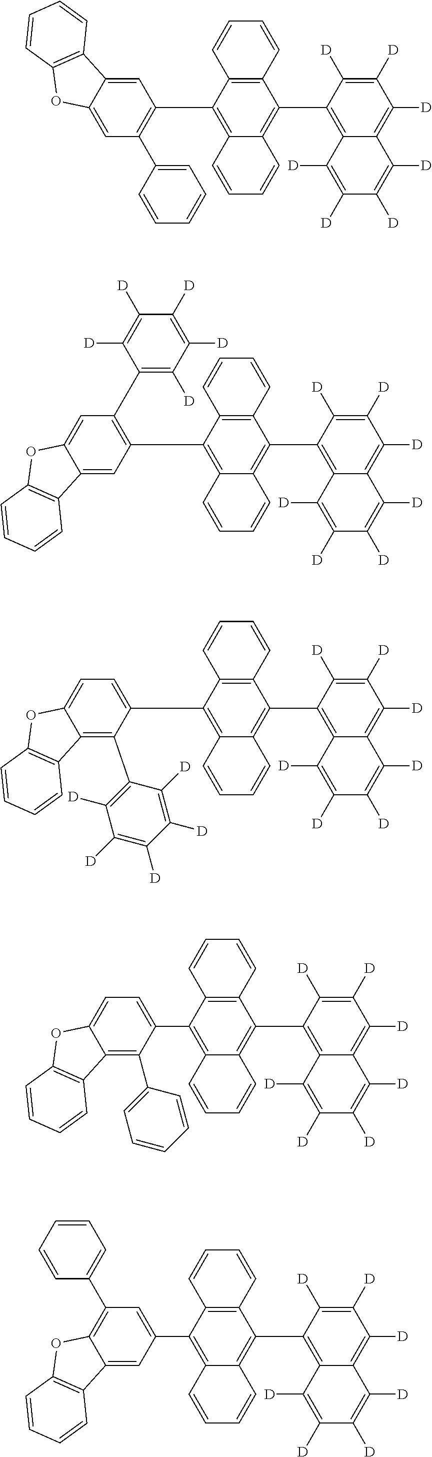

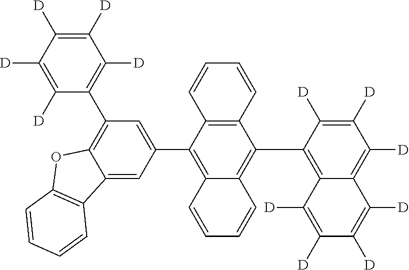 [SMILES: [2H]c1c([2H])c([2H])c(-c2cc(-c3c4ccccc4c(-c4c([2H])c([2H])c([2H])c5c([2H])c([2H])c([2H])c([2H])c45)c4ccccc34)cc3c2oc2ccccc23)c([2H])c1[2H]]

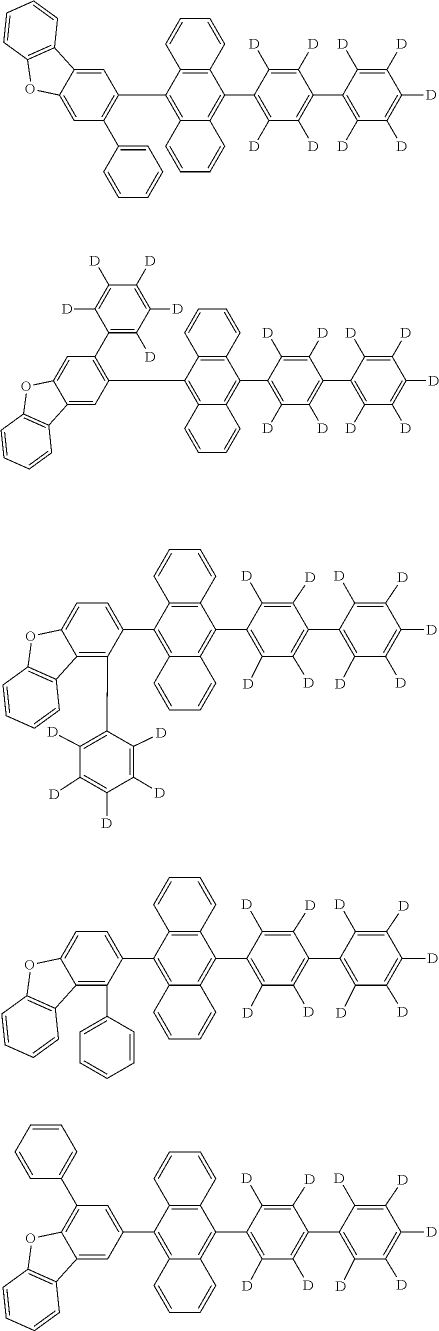

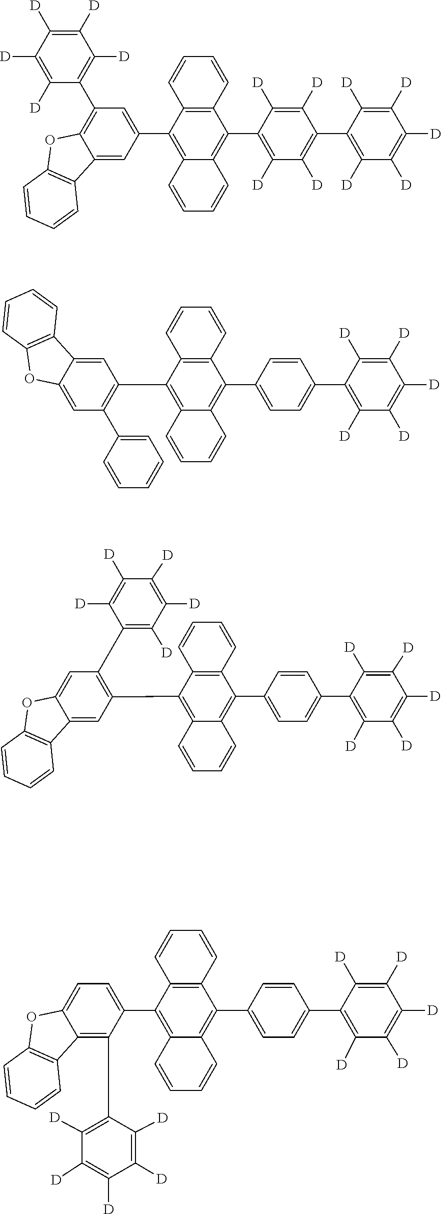

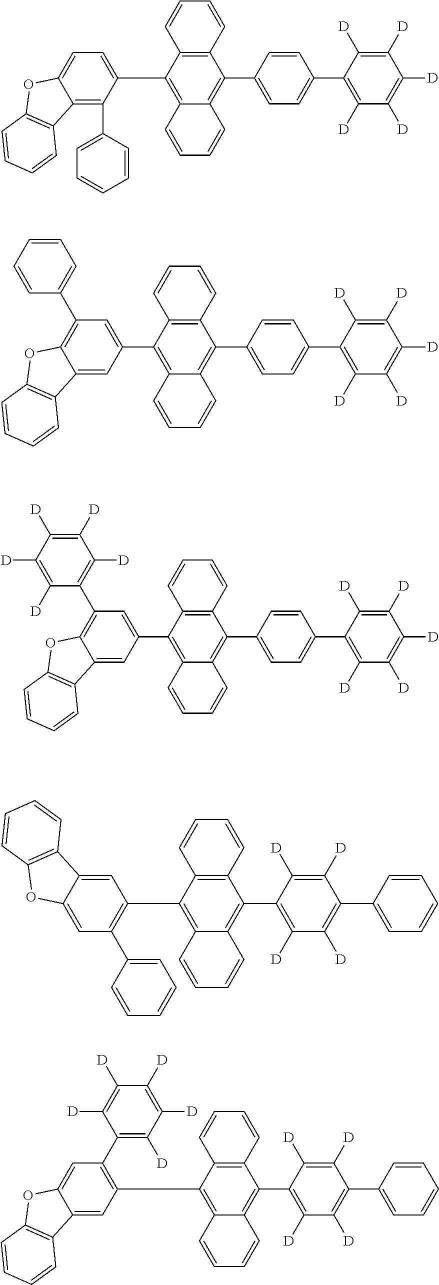

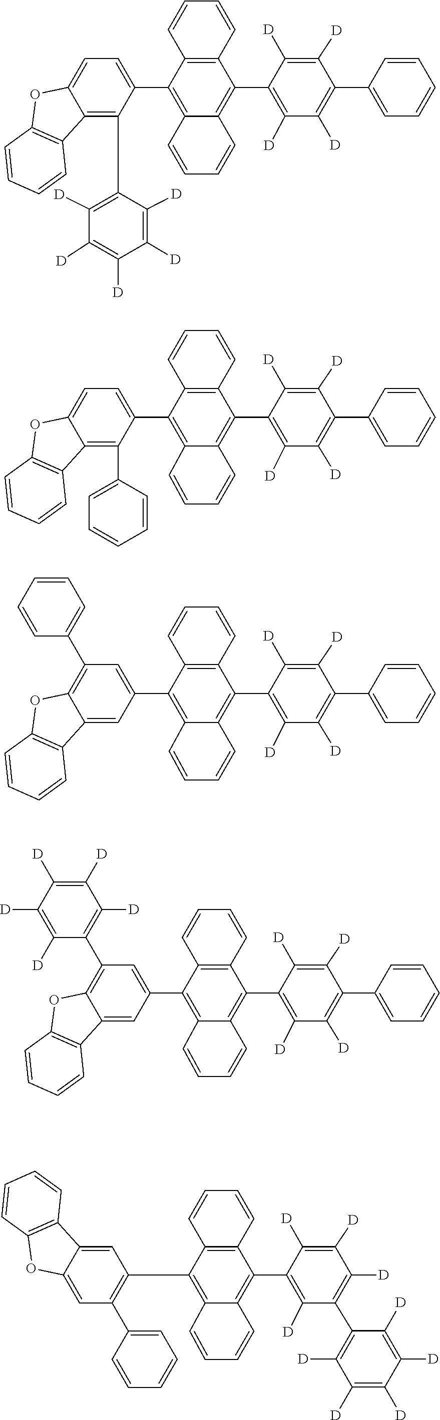

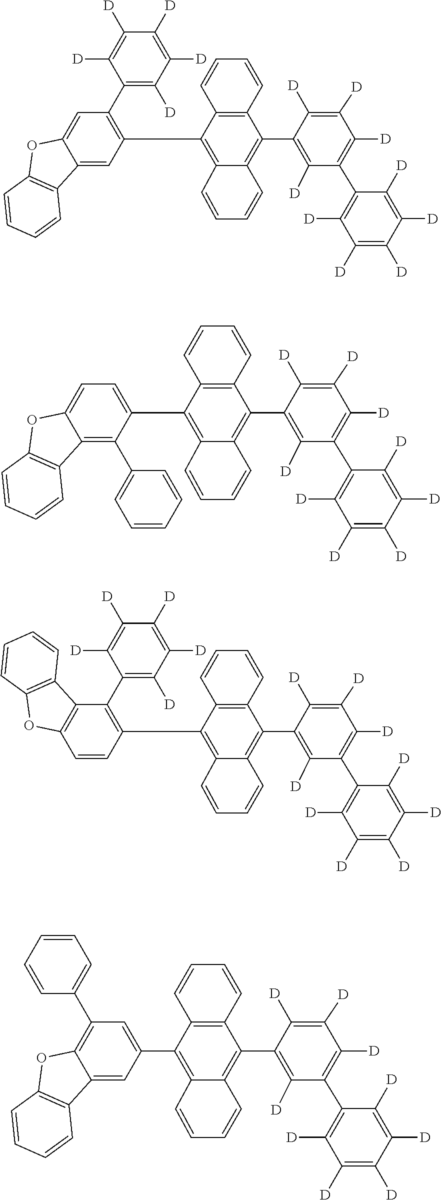

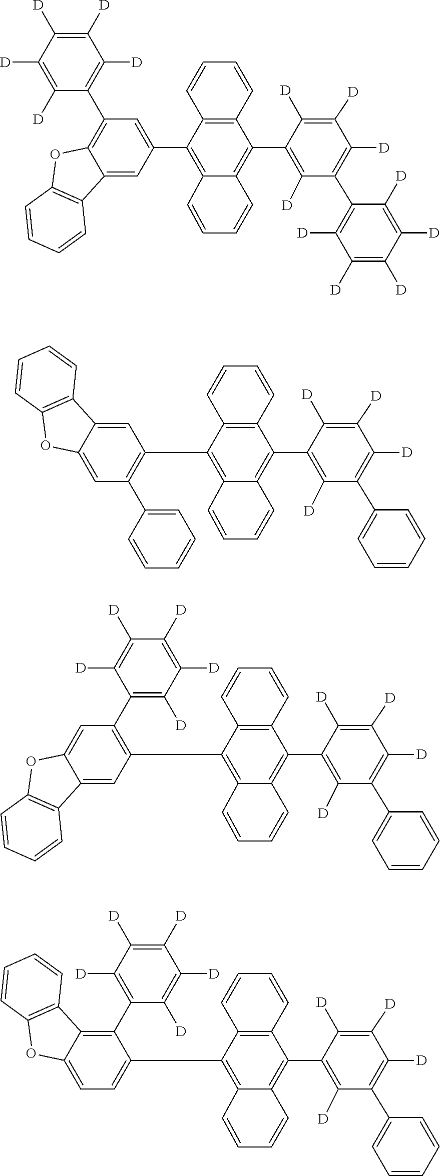

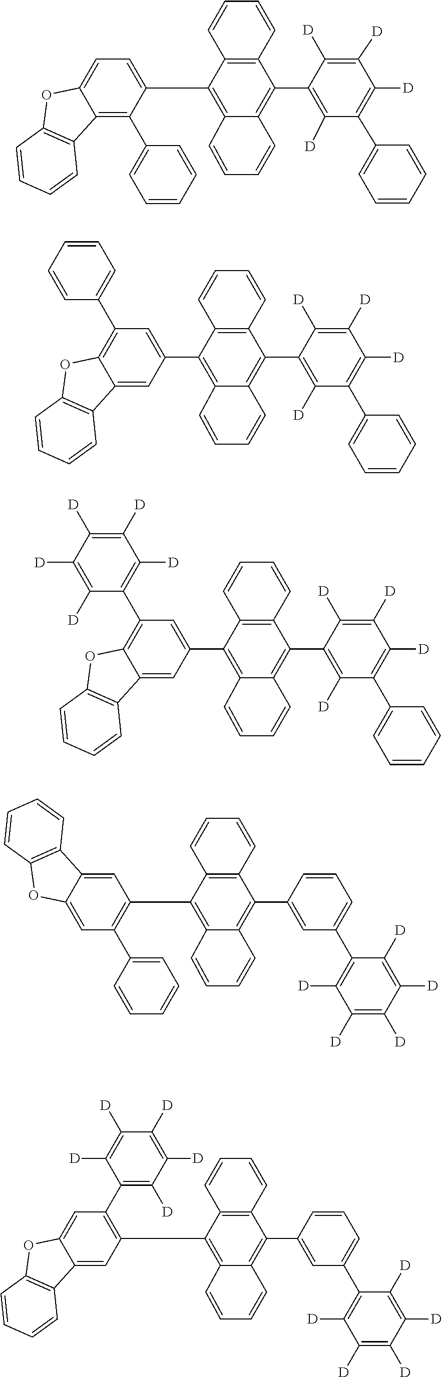

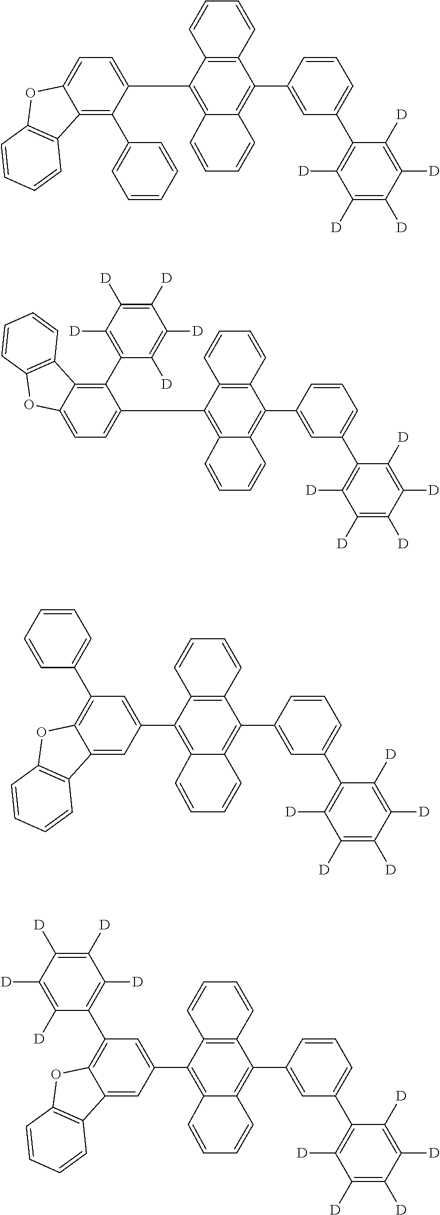 [SMILES: [2H]c1c([2H])c([2H])c(-c2cccc(-c3c4ccccc4c(-c4ccc5oc6ccccc6c5c4-c4ccccc4)c4ccccc34)c2)c([2H])c1[2H] ; [2H]c1c([2H])c([2H])c(-c2cccc(-c3c4ccccc4c(-c4ccc5oc6ccccc6c5c4-c4c([2H])c([2H])c([2H])c([2H])c4[2H])c4ccccc34)c2)c([2H])c1[2H] ; [2H]c1c([2H])c([2H])c(-c2cccc(-c3c4ccccc4c(-c4cc(-c5ccccc5)c5oc6ccccc6c5c4)c4ccccc34)c2)c([2H])c1[2H] ; [2H]c1c([2H])c([2H])c(-c2cccc(-c3c4ccccc4c(-c4cc(-c5c([2H])c([2H])c([2H])c([2H])c5[2H])c5oc6ccccc6c5c4)c4ccccc34)c2)c([2H])c1[2H]]

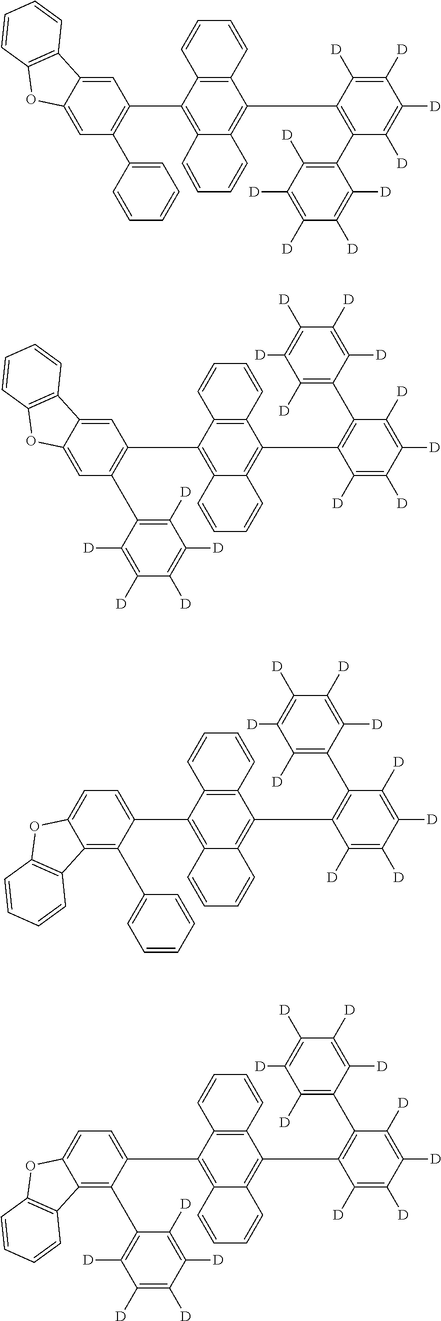

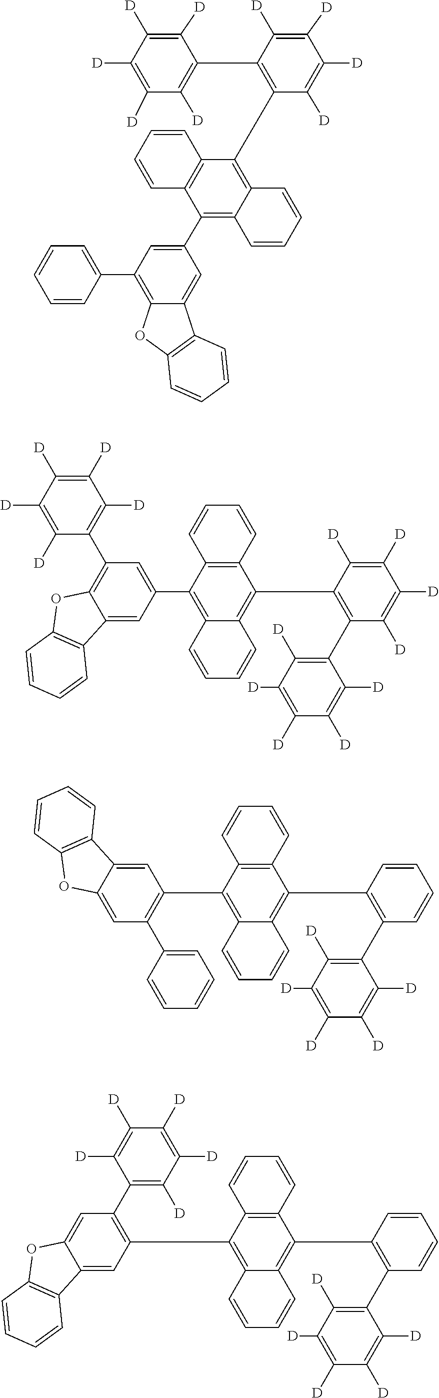

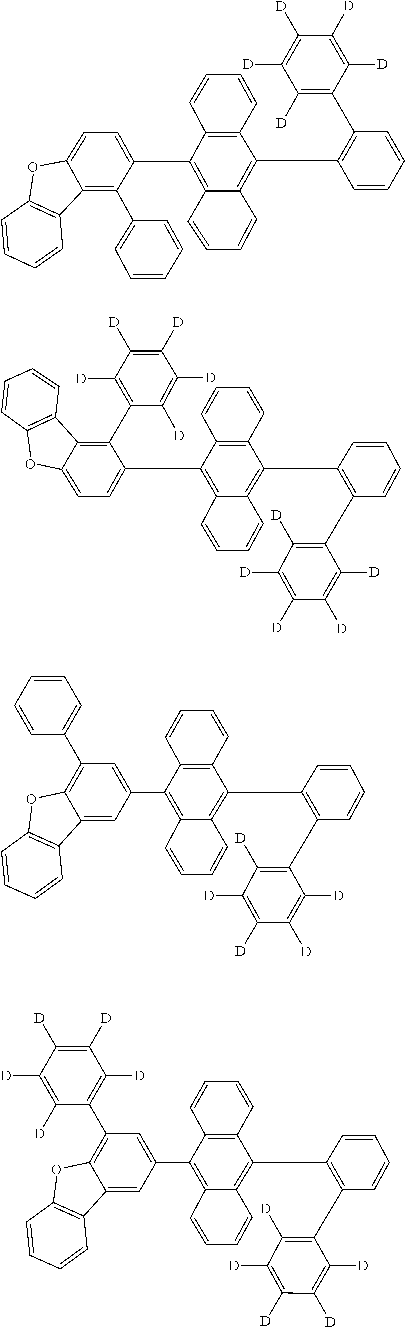

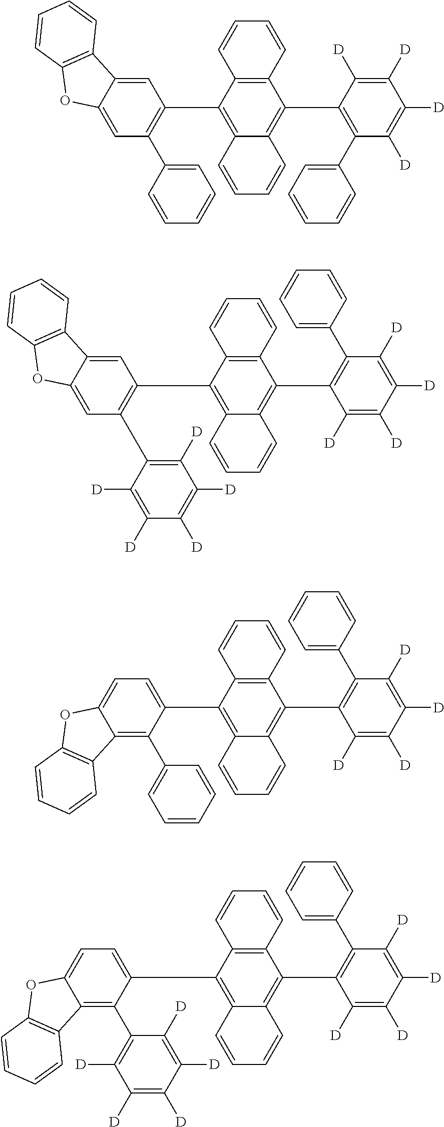 [SMILES: [2H]c1c([2H])c([2H])c(-c2c3ccccc3c(-c3cc4c(cc3-c3ccccc3)oc3ccccc34)c3ccccc23)c(-c2ccccc2)c1[2H] ; [2H]c1c([2H])c([2H])c(-c2cc3oc4ccccc4c3cc2-c2c3ccccc3c(-c3c([2H])c([2H])c([2H])c([2H])c3-c3ccccc3)c3ccccc23)c([2H])c1[2H] ; [2H]c1c([2H])c([2H])c(-c2c3ccccc3c(-c3ccc4oc5ccccc5c4c3-c3ccccc3)c3ccccc23)c(-c2ccccc2)c1[2H] ; [2H]c1c([2H])c([2H])c(-c2c(-c3c4ccccc4c(-c4c([2H])c([2H])c([2H])c([2H])c4-c4ccccc4)c4ccccc34)ccc3oc4ccccc4c23)c([2H])c1[2H]]

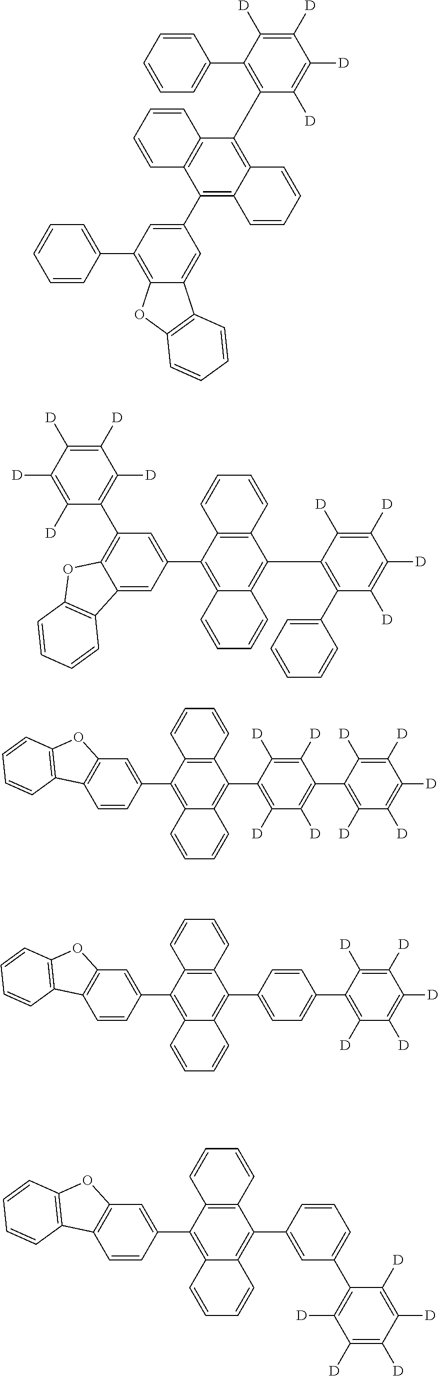

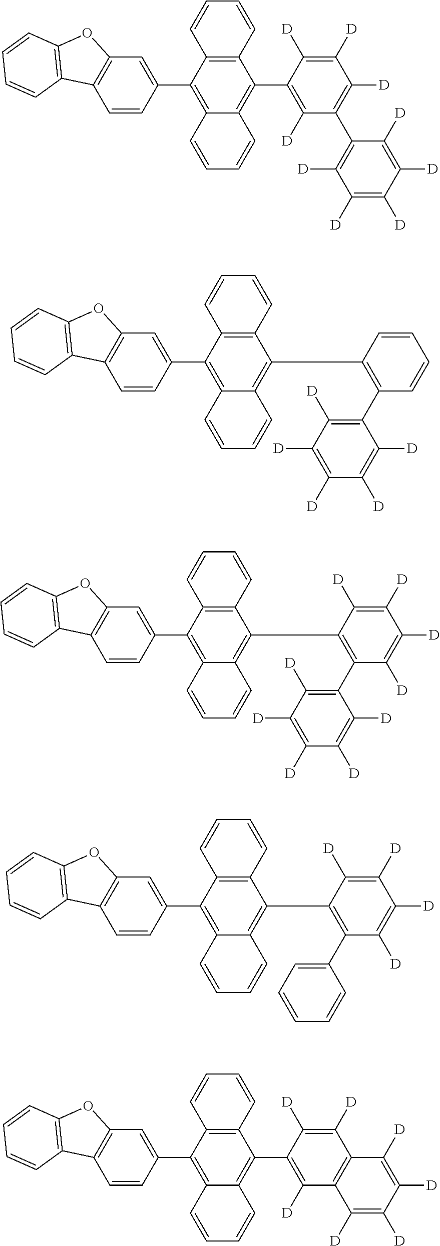

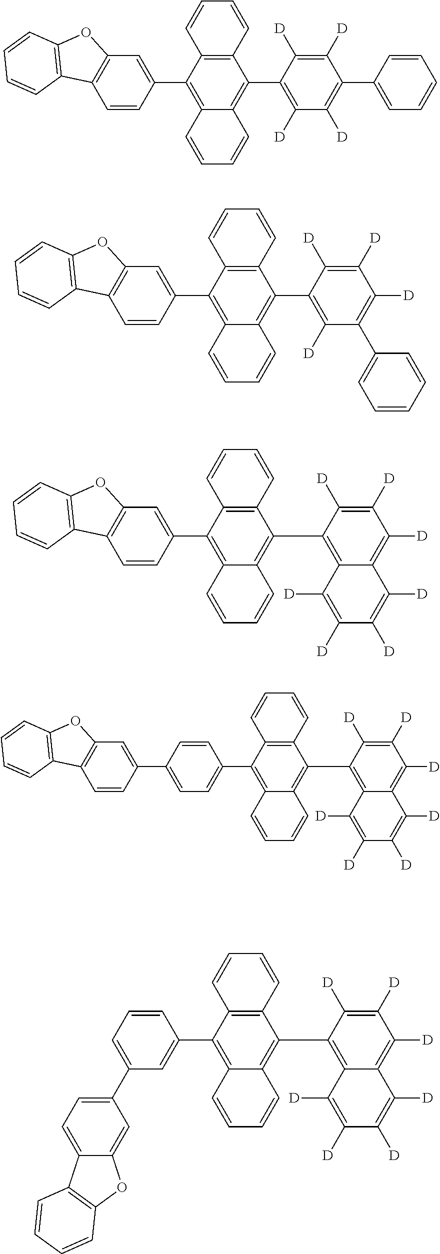

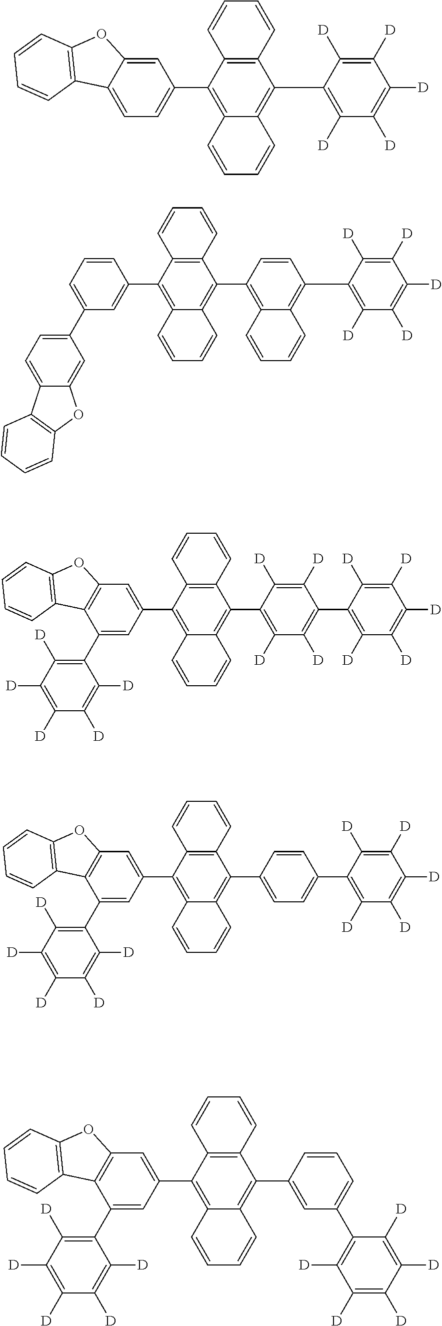

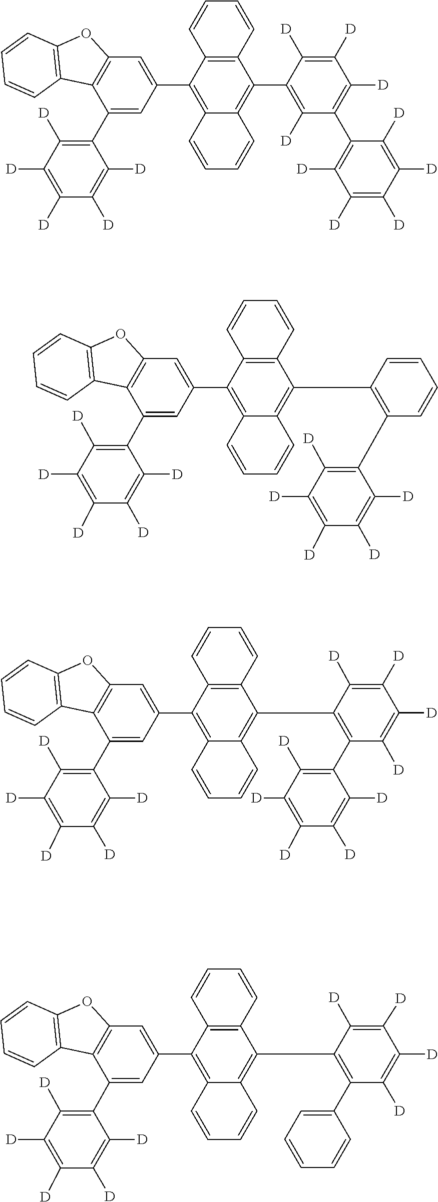

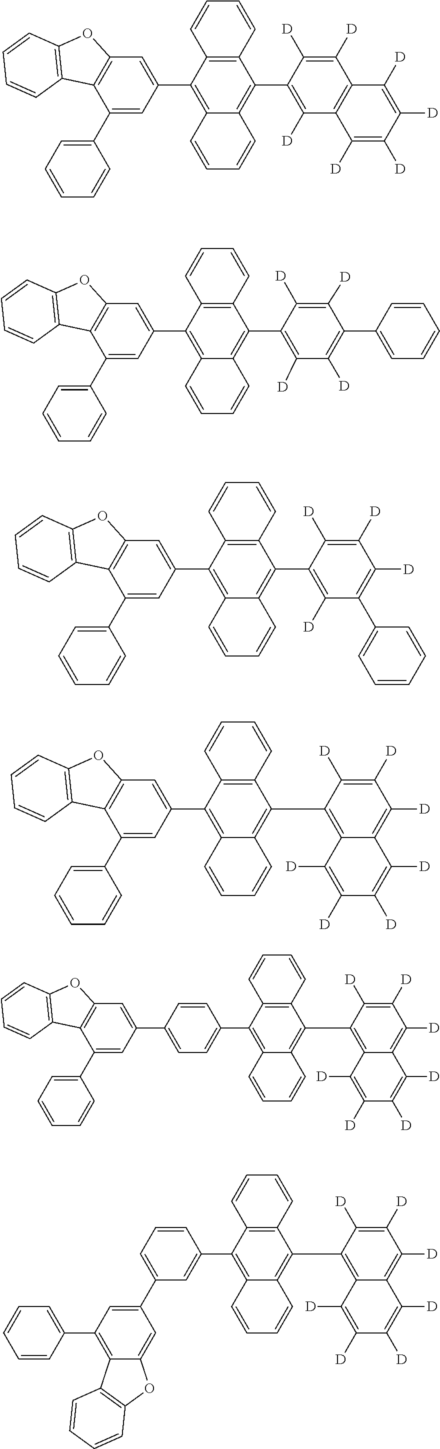

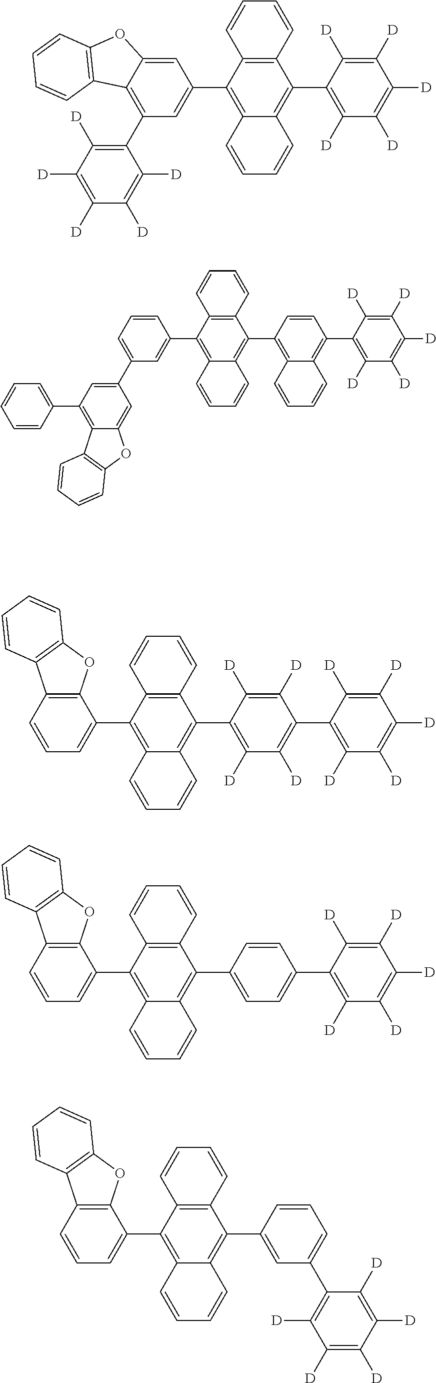

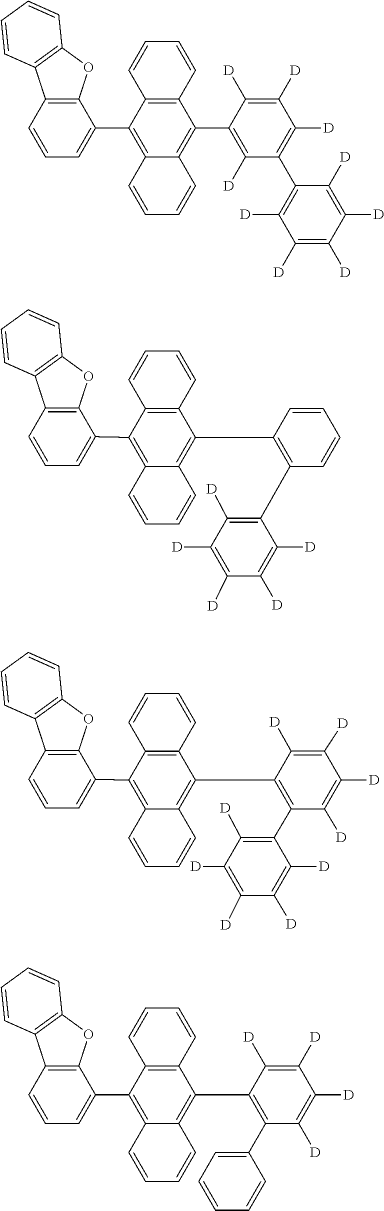 [SMILES: [2H]c1c([2H])c([2H])c(-c2c([2H])c([2H])c([2H])c(-c3c4ccccc4c(-c4cccc5c4oc4ccccc45)c4ccccc34)c2[2H])c([2H])c1[2H] ; [2H]c1c([2H])c([2H])c(-c2ccccc2-c2c3ccccc3c(-c3cccc4c3oc3ccccc34)c3ccccc23)c([2H])c1[2H] ; [2H]c1c([2H])c([2H])c(-c2c([2H])c([2H])c([2H])c([2H])c2-c2c3ccccc3c(-c3cccc4c3oc3ccccc34)c3ccccc23)c([2H])c1[2H] ; [2H]c1c([2H])c([2H])c(-c2c3ccccc3c(-c3cccc4c3oc3ccccc34)c3ccccc23)c(-c2ccccc2)c1[2H]]

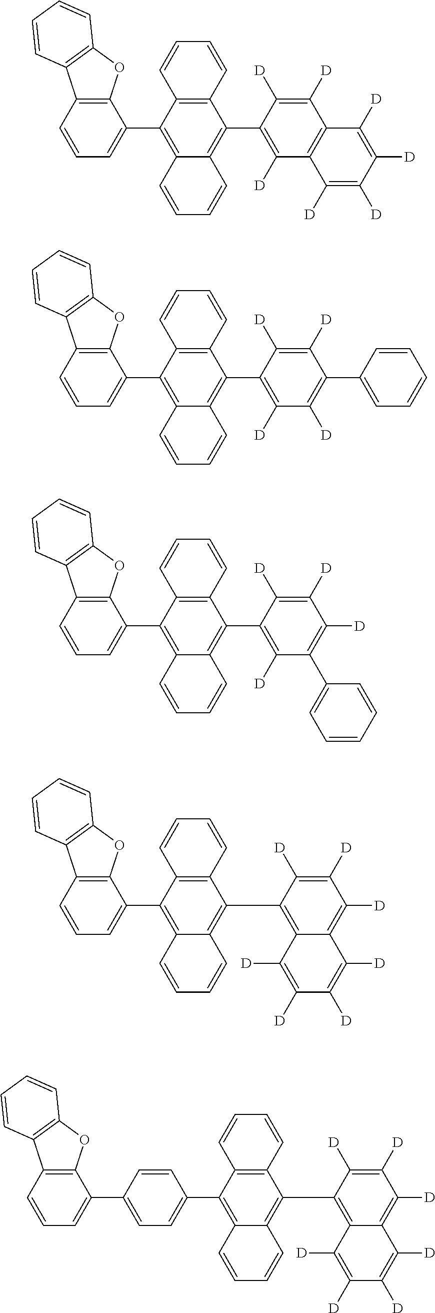 [SMILES: [2H]c1c([2H])c([2H])c2c([2H])c(-c3c4ccccc4c(-c4cccc5c4oc4ccccc45)c4ccccc34)c([2H])c([2H])c2c1[2H] ; [2H]c1c([2H])c(-c2c3ccccc3c(-c3cccc4c3oc3ccccc34)c3ccccc23)c([2H])c([2H])c1-c1ccccc1 ; [2H]c1c([2H])c(-c2ccccc2)c([2H])c(-c2c3ccccc3c(-c3cccc4c3oc3ccccc34)c3ccccc23)c1[2H] ; [2H]c1c([2H])c([2H])c2c(-c3c4ccccc4c(-c4cccc5c4oc4ccccc45)c4ccccc34)c([2H])c([2H])c([2H])c2c1[2H] ; [2H]c1c([2H])c([2H])c2c(-c3c4ccccc4c(-c4ccc(-c5cccc6c5oc5ccccc56)cc4)c4ccccc34)c([2H])c([2H])c([2H])c2c1[2H]]

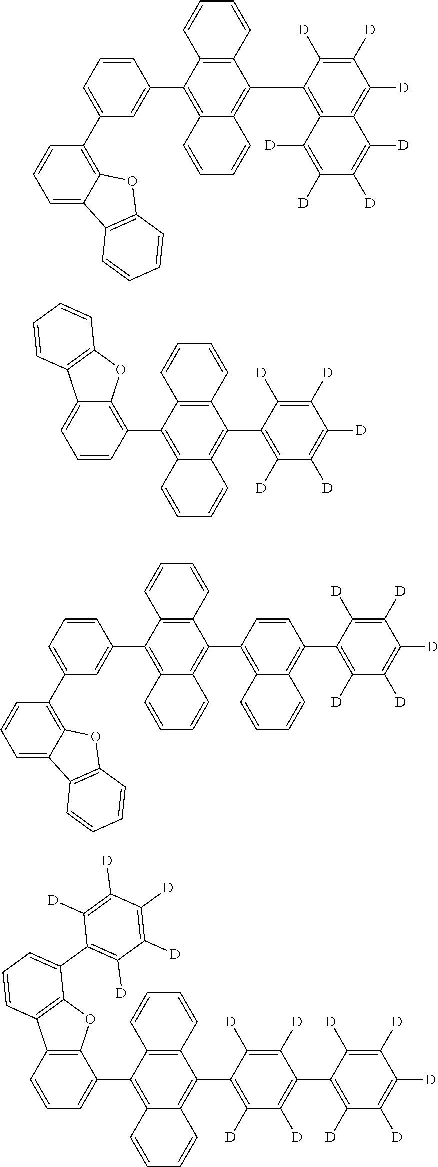

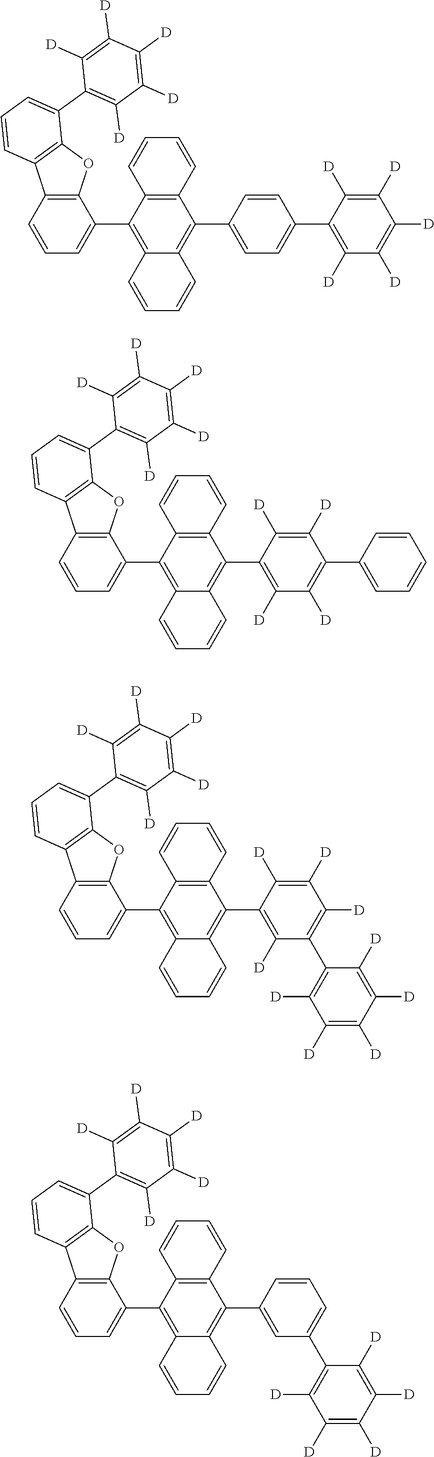

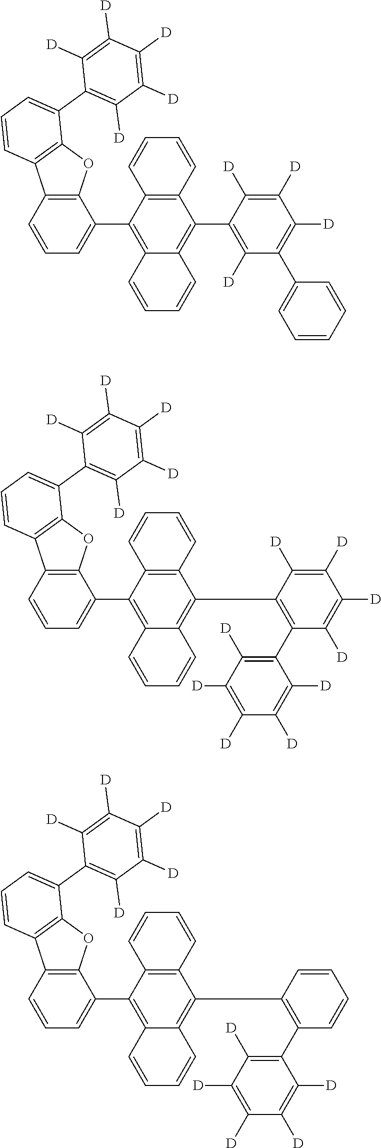 [SMILES: [2H]c1c([2H])c([2H])c(-c2cccc3c2oc2c(-c4c5ccccc5c(-c5c([2H])c([2H])c([2H])c(-c6ccccc6)c5[2H])c5ccccc45)cccc23)c([2H])c1[2H] ; [2H]c1c([2H])c([2H])c(-c2c([2H])c([2H])c([2H])c([2H])c2-c2c3ccccc3c(-c3cccc4c3oc3c(-c5c([2H])c([2H])c([2H])c([2H])c5[2H])cccc34)c3ccccc23)c([2H])c1[2H] ; [2H]c1c([2H])c([2H])c(-c2ccccc2-c2c3ccccc3c(-c3cccc4c3oc3c(-c5c([2H])c([2H])c([2H])c([2H])c5[2H])cccc34)c3ccccc23)c([2H])c1[2H]]

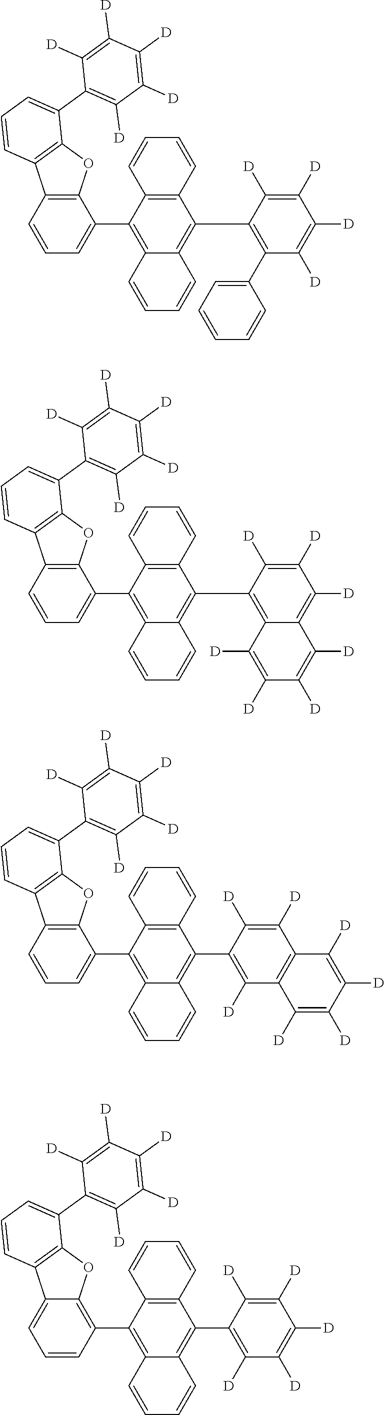

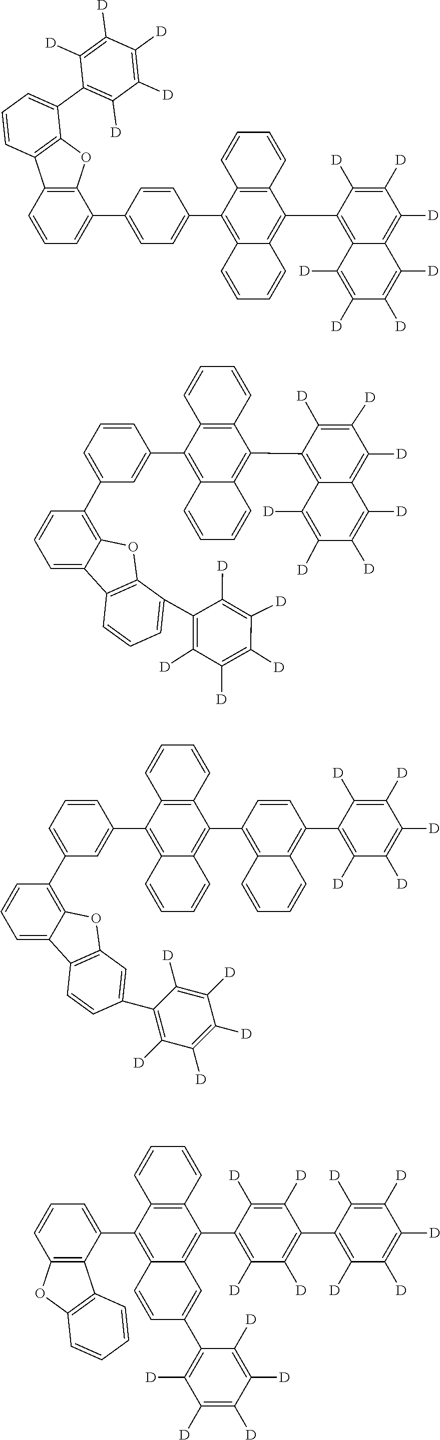

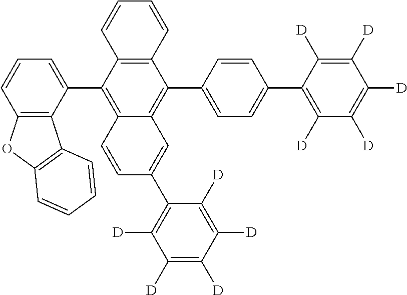 [SMILES: [2H]c1c([2H])c([2H])c(-c2ccc(-c3c4ccccc4c(-c4cccc5oc6ccccc6c45)c4ccc(-c5c([2H])c([2H])c([2H])c([2H])c5[2H])cc34)cc2)c([2H])c1[2H]]

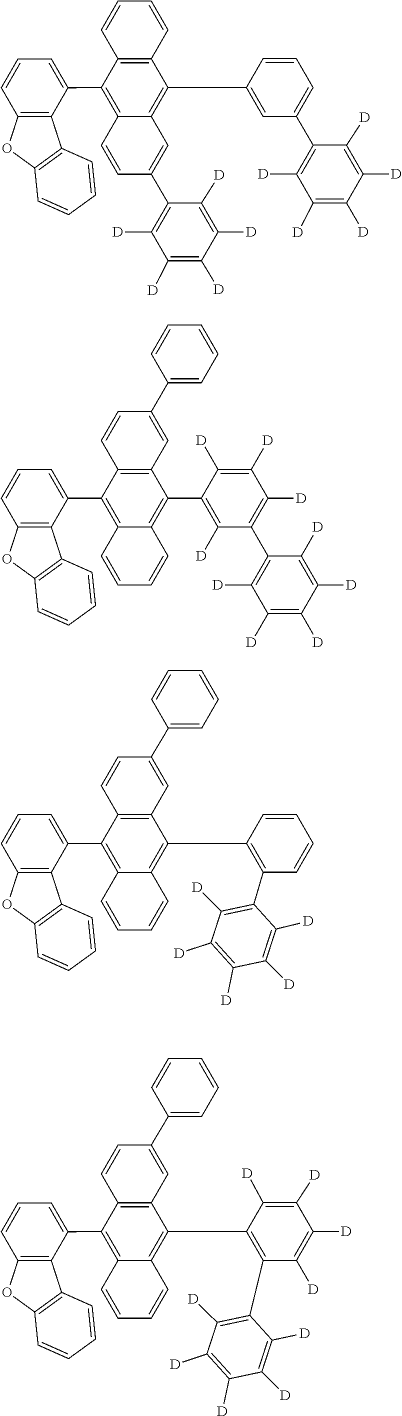

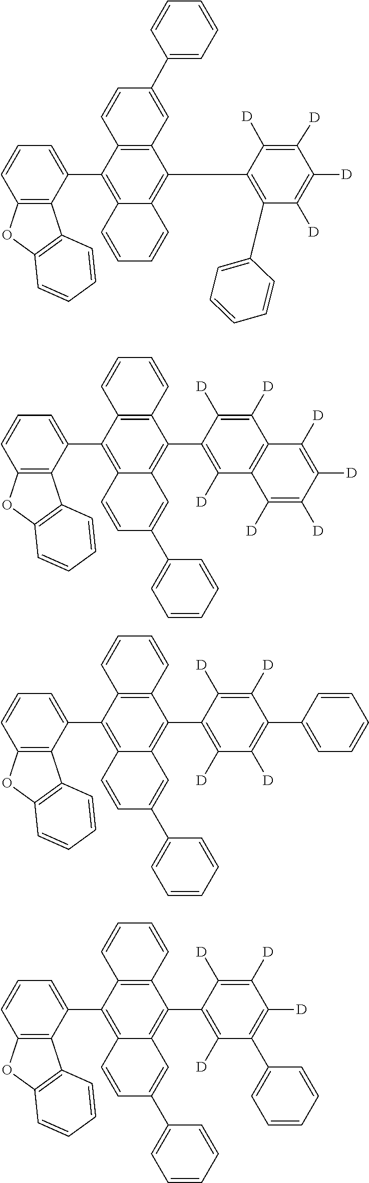

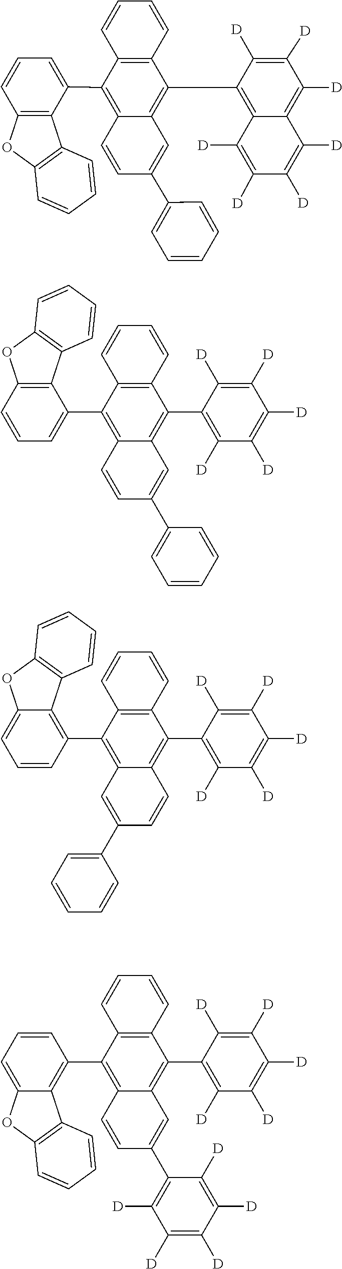 [SMILES: [2H]c1c([2H])c([2H])c2c(-c3c4ccccc4c(-c4cccc5oc6ccccc6c45)c4ccc(-c5ccccc5)cc34)c([2H])c([2H])c([2H])c2c1[2H] ; [2H]c1c([2H])c([2H])c(-c2c3ccccc3c(-c3cccc4oc5ccccc5c34)c3ccc(-c4ccccc4)cc23)c([2H])c1[2H] ; [2H]c1c([2H])c([2H])c(-c2c3ccccc3c(-c3cccc4oc5ccccc5c34)c3cc(-c4ccccc4)ccc23)c([2H])c1[2H] ; [2H]c1c([2H])c([2H])c(-c2ccc3c(-c4cccc5oc6ccccc6c45)c4ccccc4c(-c4c([2H])c([2H])c([2H])c([2H])c4[2H])c3c2)c([2H])c1[2H]]

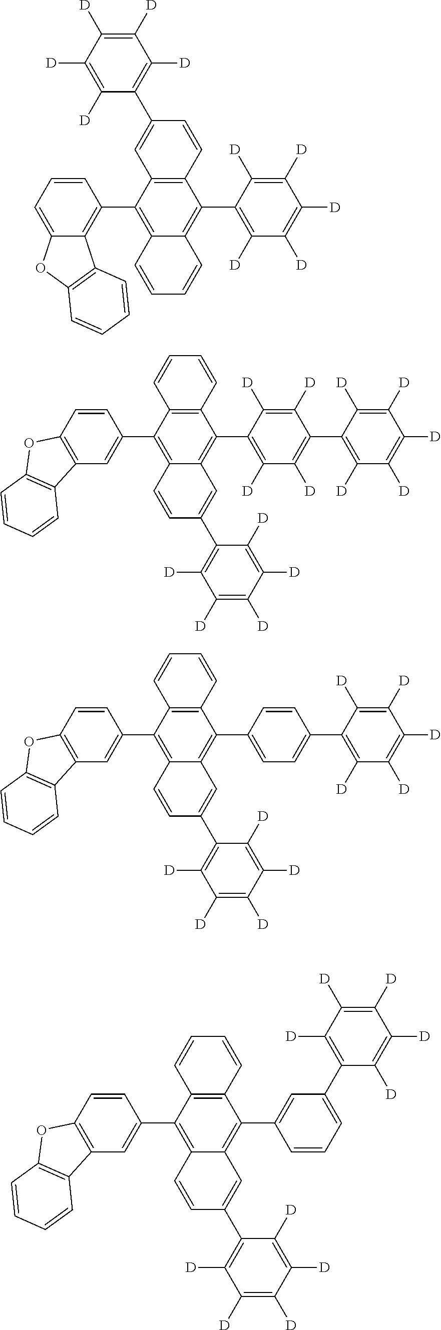

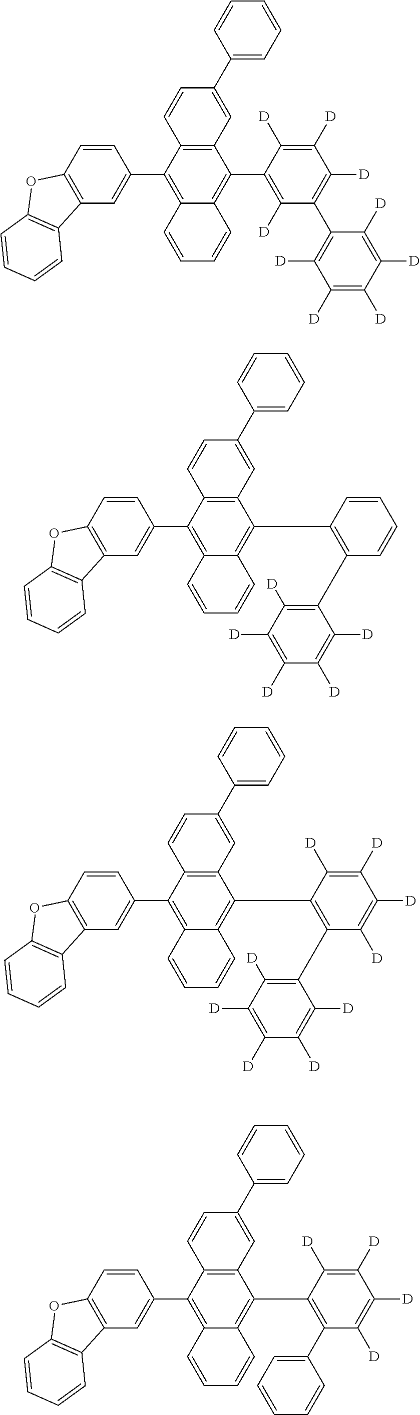

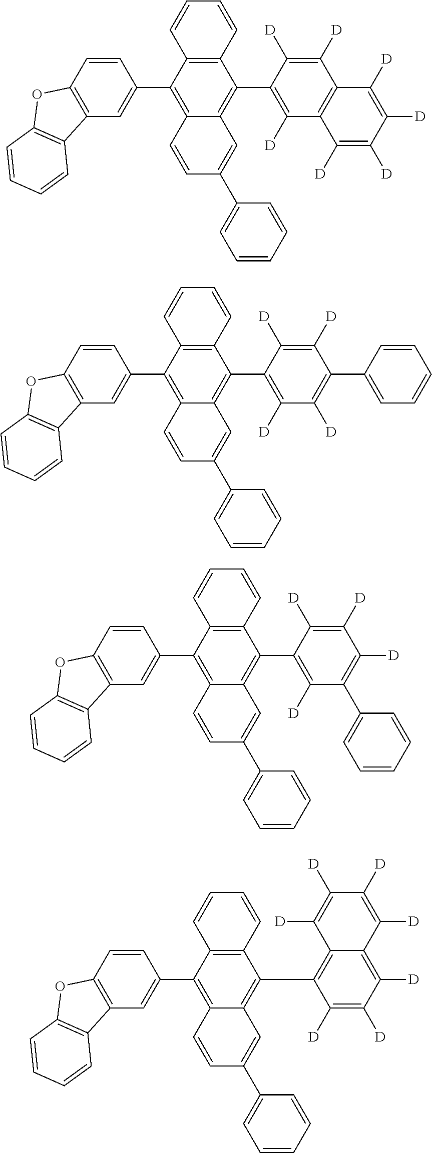

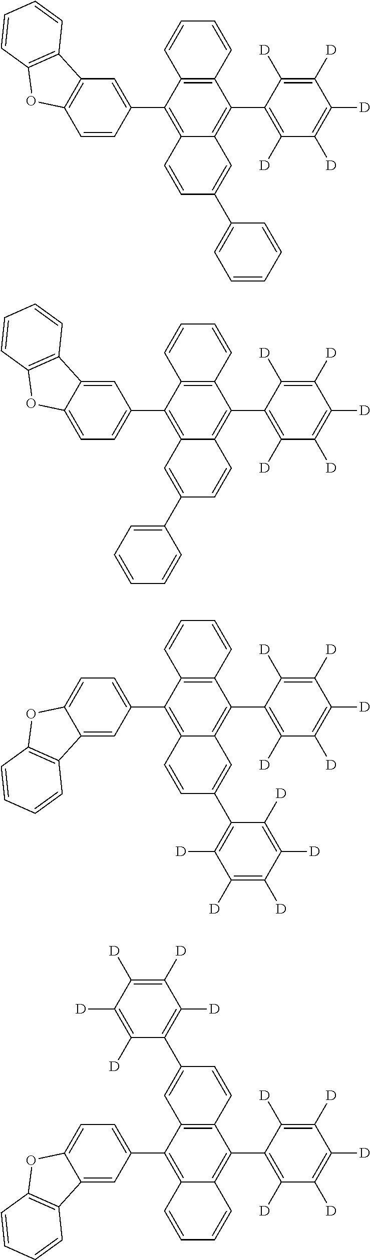

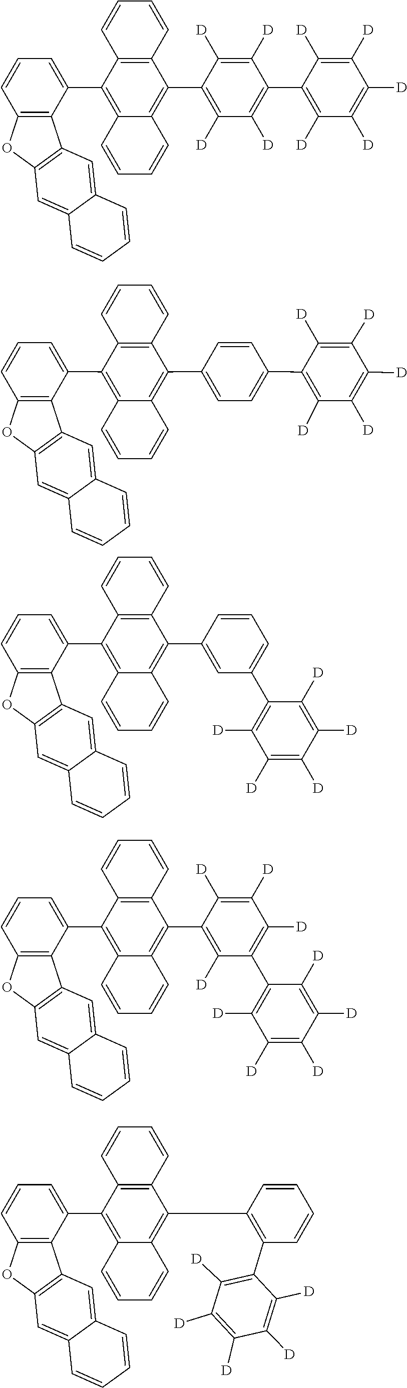

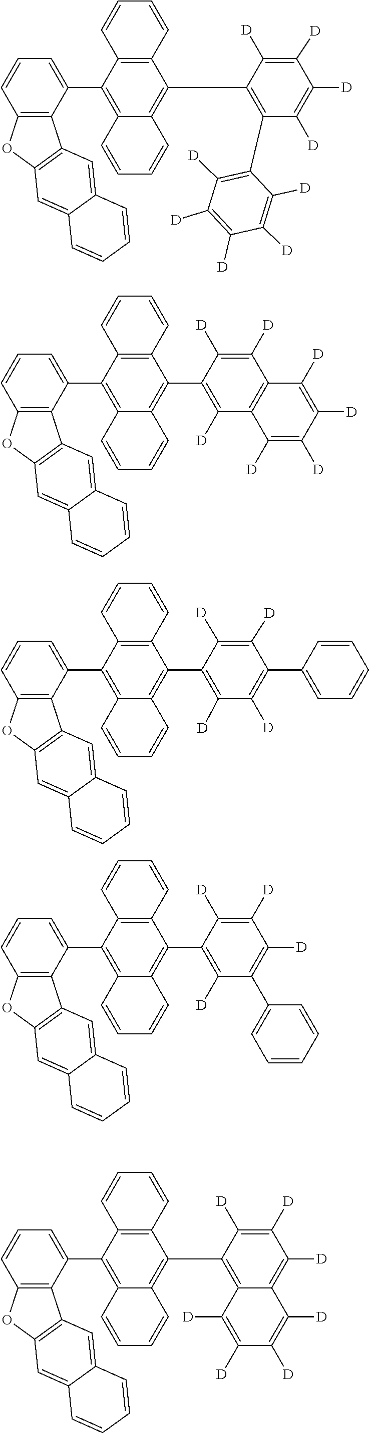

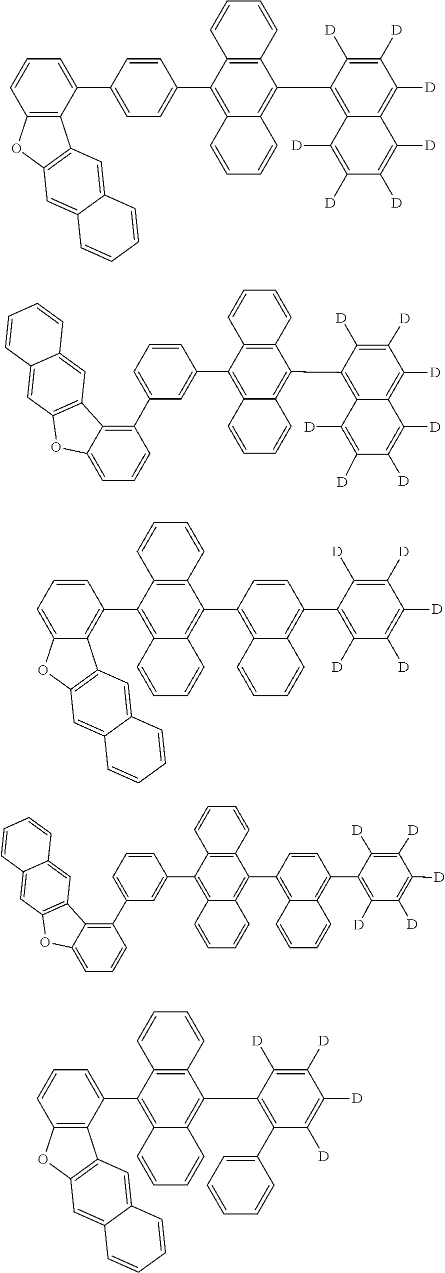

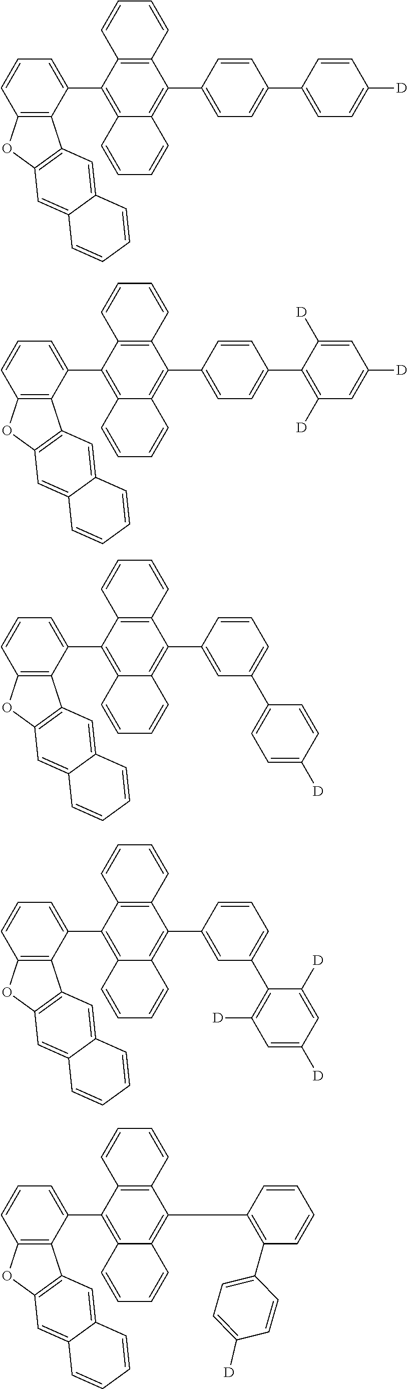

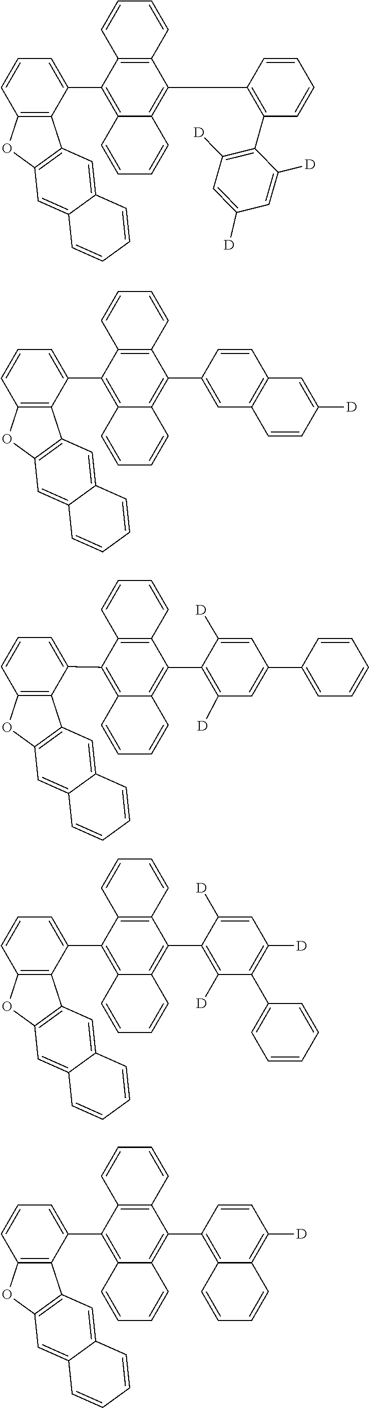

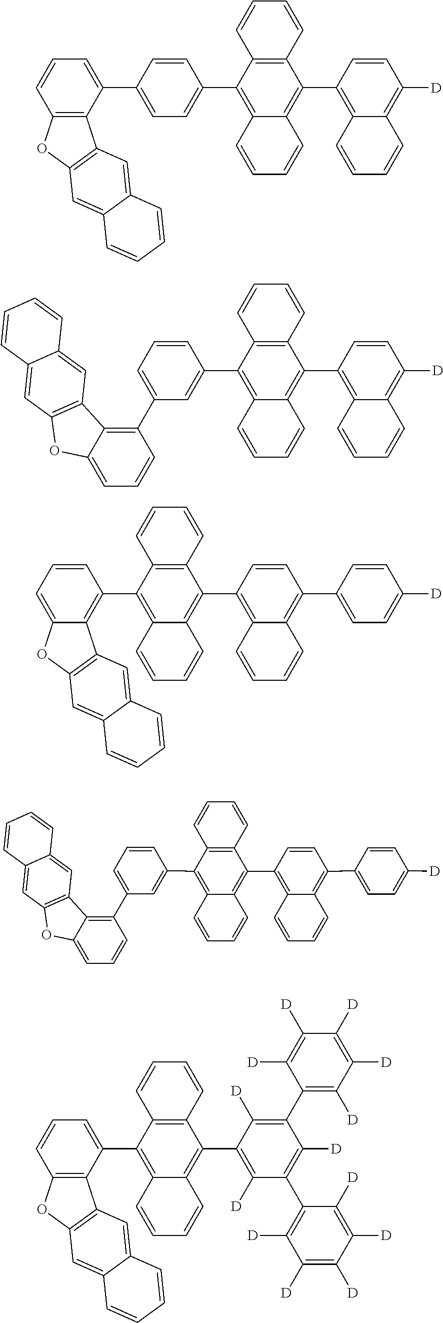

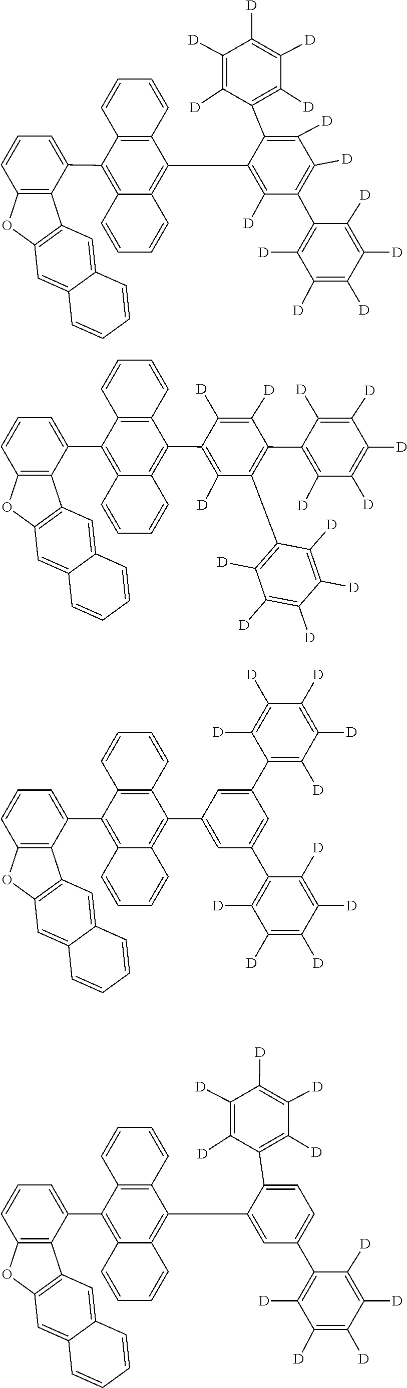

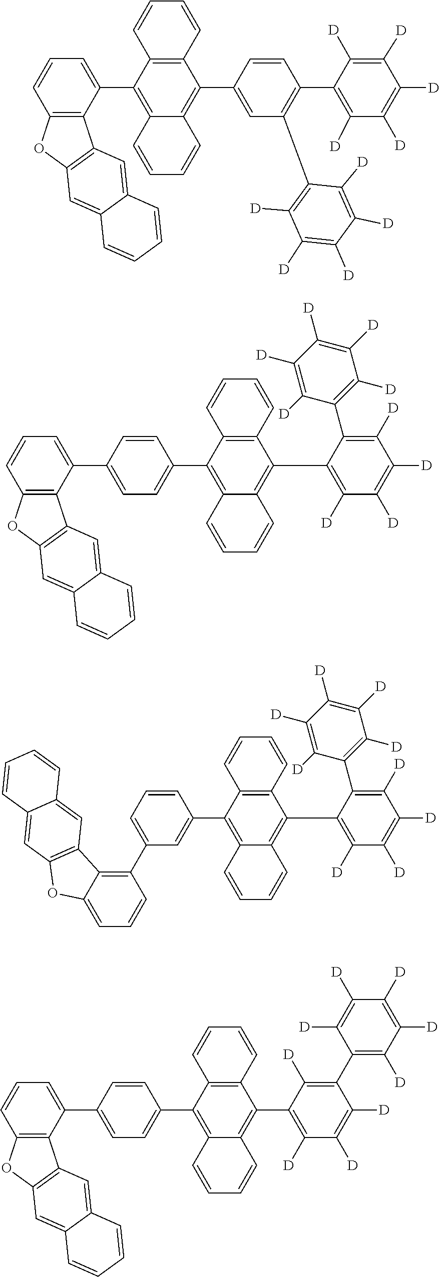

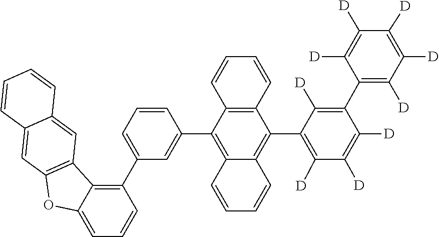 [SMILES: [2H]c1c([2H])c([2H])c(-c2c([2H])c([2H])c([2H])c(-c3c4ccccc4c(-c4cccc(-c5cccc6oc7cc8ccccc8cc7c56)c4)c4ccccc34)c2[2H])c([2H])c1[2H]]

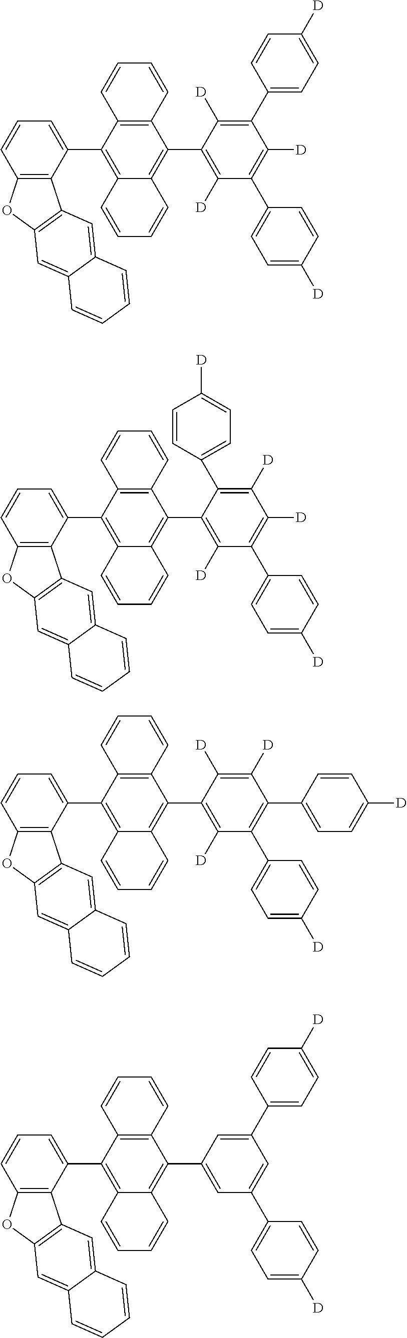

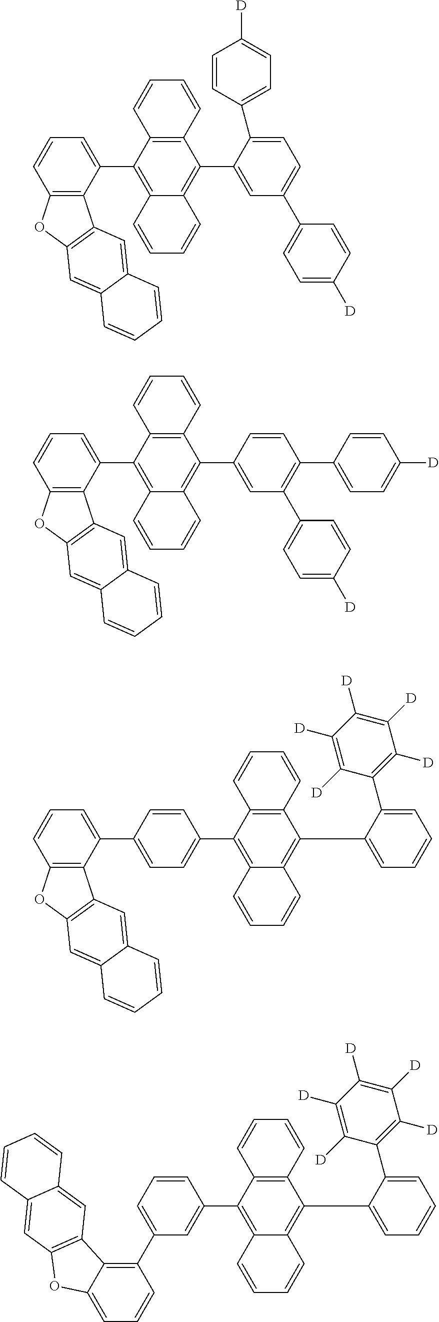

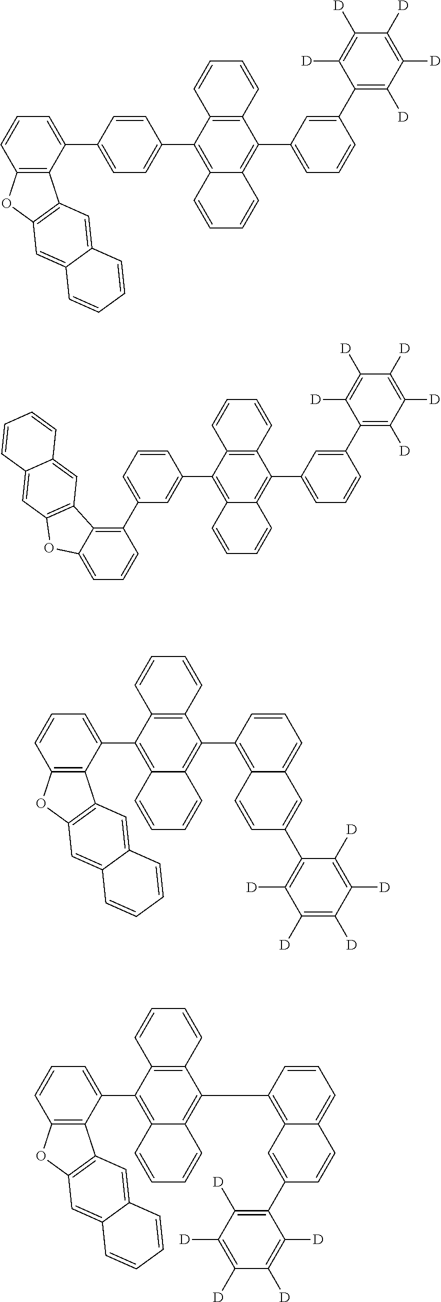 [SMILES: [2H]c1c([2H])c([2H])c(-c2cccc(-c3c4ccccc4c(-c4ccc(-c5cccc6oc7cc8ccccc8cc7c56)cc4)c4ccccc34)c2)c([2H])c1[2H] ; [2H]c1c([2H])c([2H])c(-c2cccc(-c3c4ccccc4c(-c4cccc(-c5cccc6oc7cc8ccccc8cc7c56)c4)c4ccccc34)c2)c([2H])c1[2H] ; [2H]c1c([2H])c([2H])c(-c2ccc3c(-c4c5ccccc5c(-c5cccc6oc7cc8ccccc8cc7c56)c5ccccc45)cccc3c2)c([2H])c1[2H] ; [2H]c1c([2H])c([2H])c(-c2ccc3cccc(-c4c5ccccc5c(-c5cccc6oc7cc8ccccc8cc7c56)c5ccccc45)c3c2)c([2H])c1[2H]]

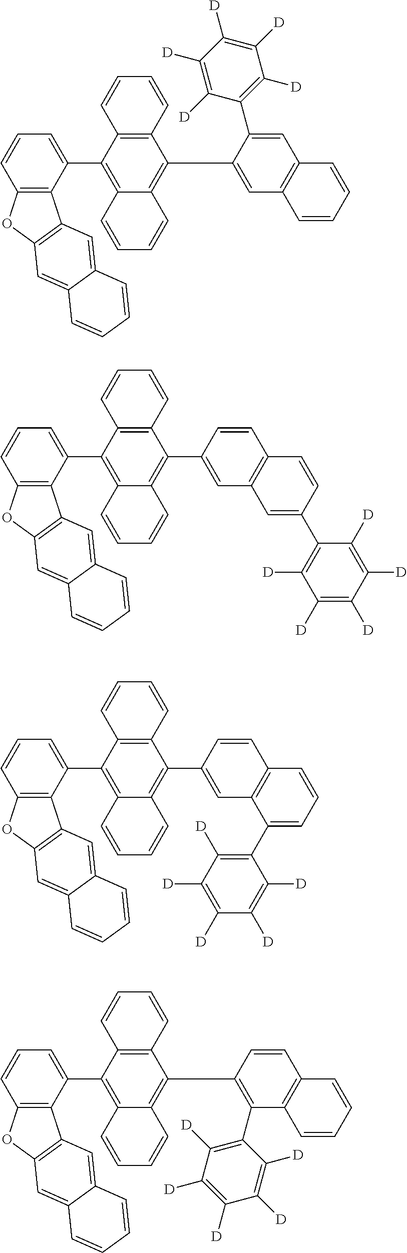

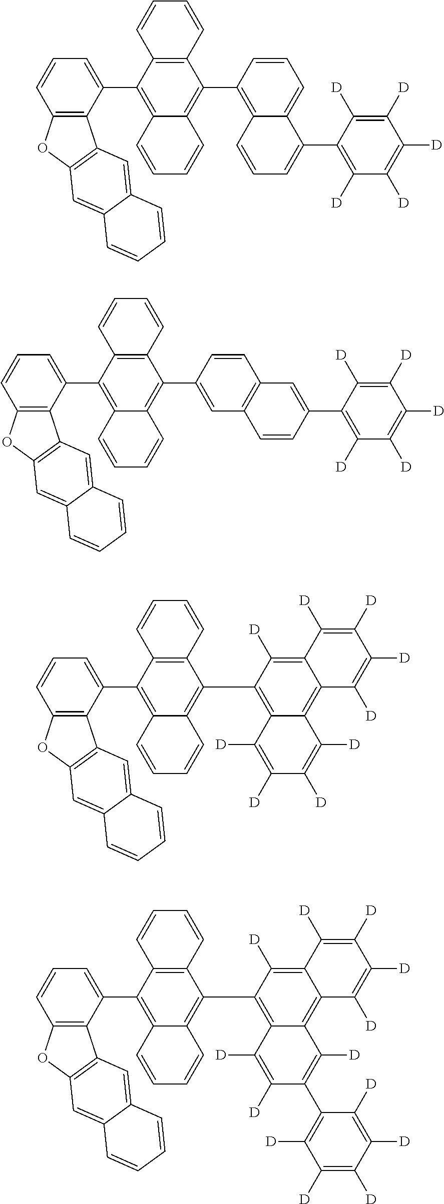

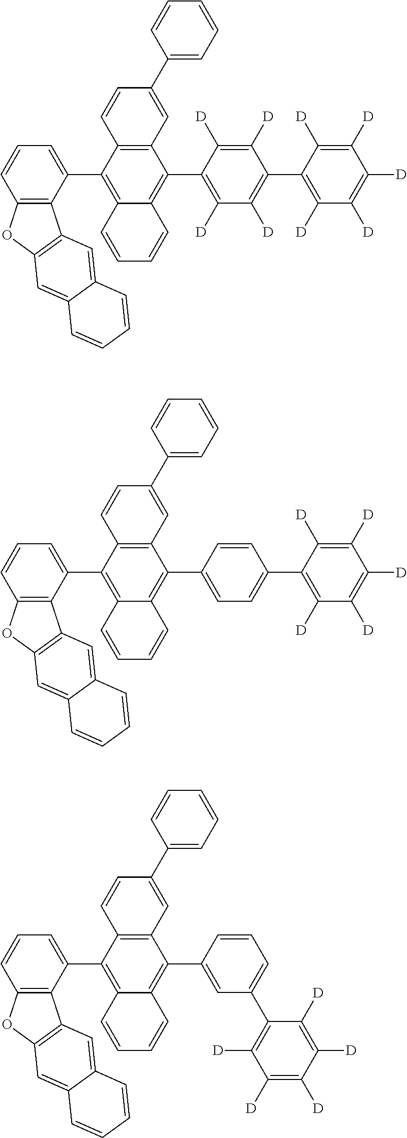 [SMILES: [2H]c1c([2H])c([2H])c(-c2c([2H])c([2H])c(-c3c4ccccc4c(-c4cccc5oc6cc7ccccc7cc6c45)c4ccc(-c5ccccc5)cc34)c([2H])c2[2H])c([2H])c1[2H] ; [2H]c1c([2H])c([2H])c(-c2ccc(-c3c4ccccc4c(-c4cccc5oc6cc7ccccc7cc6c45)c4ccc(-c5ccccc5)cc34)cc2)c([2H])c1[2H] ; [2H]c1c([2H])c([2H])c(-c2cccc(-c3c4ccccc4c(-c4cccc5oc6cc7ccccc7cc6c45)c4ccc(-c5ccccc5)cc34)c2)c([2H])c1[2H]]

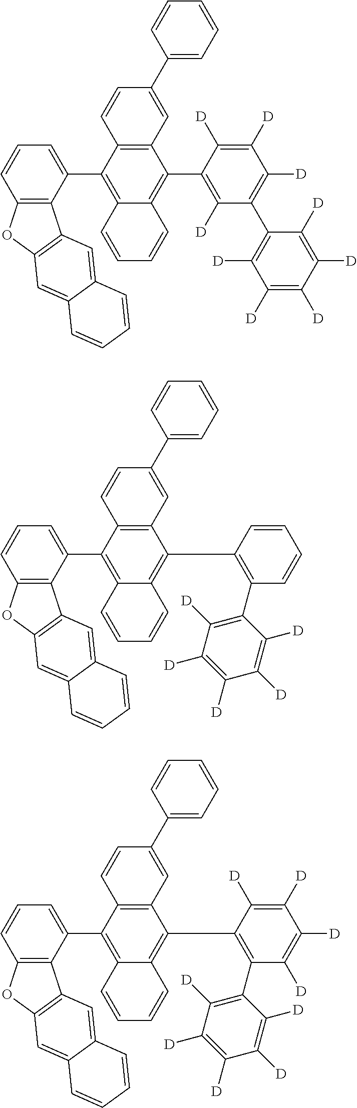 [SMILES: [2H]c1c([2H])c([2H])c(-c2c([2H])c([2H])c([2H])c(-c3c4ccccc4c(-c4cccc5oc6cc7ccccc7cc6c45)c4ccc(-c5ccccc5)cc34)c2[2H])c([2H])c1[2H] ; [2H]c1c([2H])c([2H])c(-c2ccccc2-c2c3ccccc3c(-c3cccc4oc5cc6ccccc6cc5c34)c3ccc(-c4ccccc4)cc23)c([2H])c1[2H] ; [2H]c1c([2H])c([2H])c(-c2c([2H])c([2H])c([2H])c([2H])c2-c2c3ccccc3c(-c3cccc4oc5cc6ccccc6cc5c34)c3ccc(-c4ccccc4)cc23)c([2H])c1[2H]]

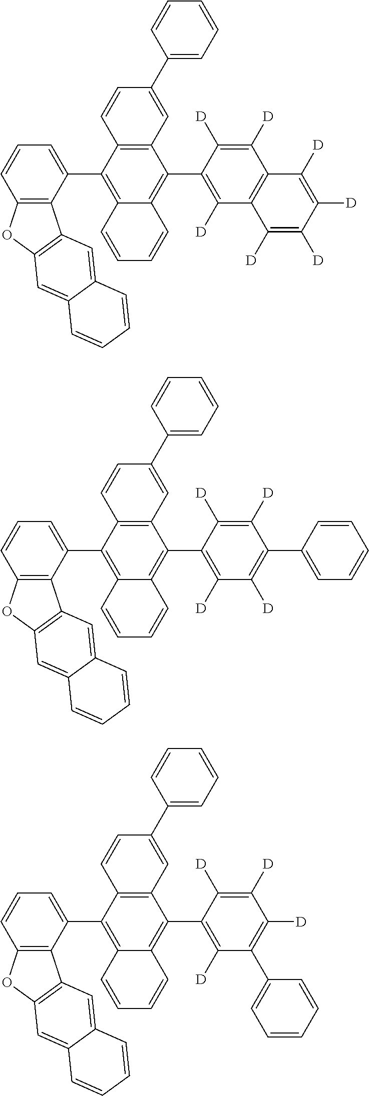

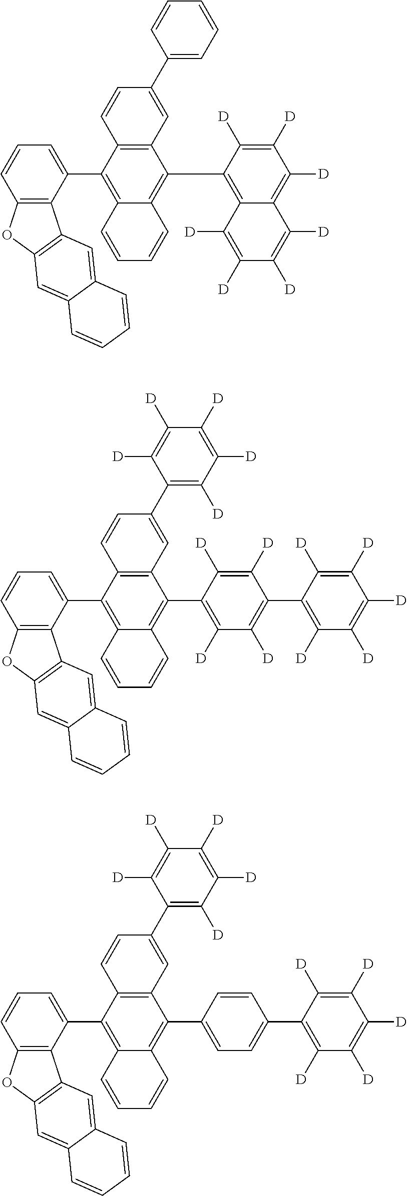 [SMILES: [2H]c1c([2H])c([2H])c2c(-c3c4ccccc4c(-c4cccc5oc6cc7ccccc7cc6c45)c4ccc(-c5ccccc5)cc34)c([2H])c([2H])c([2H])c2c1[2H] ; [2H]c1c([2H])c([2H])c(-c2ccc3c(-c4cccc5oc6cc7ccccc7cc6c45)c4ccccc4c(-c4c([2H])c([2H])c(-c5c([2H])c([2H])c([2H])c([2H])c5[2H])c([2H])c4[2H])c3c2)c([2H])c1[2H] ; [2H]c1c([2H])c([2H])c(-c2ccc(-c3c4ccccc4c(-c4cccc5oc6cc7ccccc7cc6c45)c4ccc(-c5c([2H])c([2H])c([2H])c([2H])c5[2H])cc34)cc2)c([2H])c1[2H]]

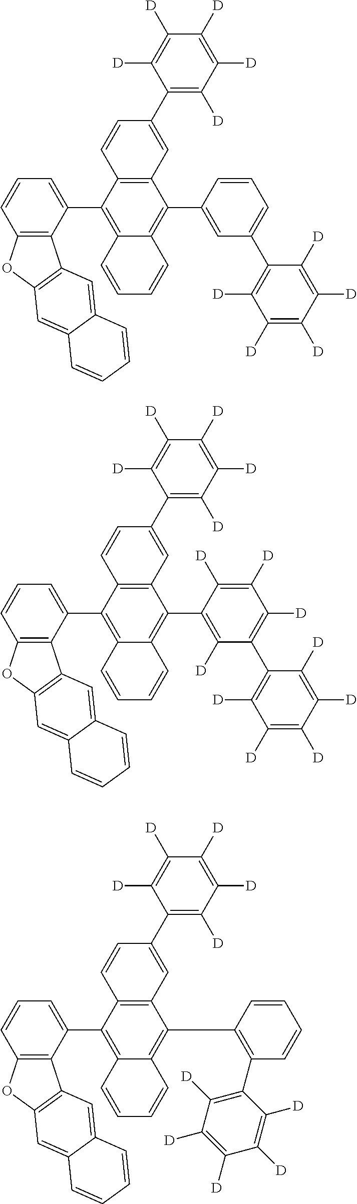 [SMILES: [2H]c1c([2H])c([2H])c(-c2cccc(-c3c4ccccc4c(-c4cccc5oc6cc7ccccc7cc6c45)c4ccc(-c5c([2H])c([2H])c([2H])c([2H])c5[2H])cc34)c2)c([2H])c1[2H] ; [2H]c1c([2H])c([2H])c(-c2ccc3c(-c4cccc5oc6cc7ccccc7cc6c45)c4ccccc4c(-c4c([2H])c([2H])c([2H])c(-c5c([2H])c([2H])c([2H])c([2H])c5[2H])c4[2H])c3c2)c([2H])c1[2H] ; [2H]c1c([2H])c([2H])c(-c2ccc3c(-c4cccc5oc6cc7ccccc7cc6c45)c4ccccc4c(-c4ccccc4-c4c([2H])c([2H])c([2H])c([2H])c4[2H])c3c2)c([2H])c1[2H]]

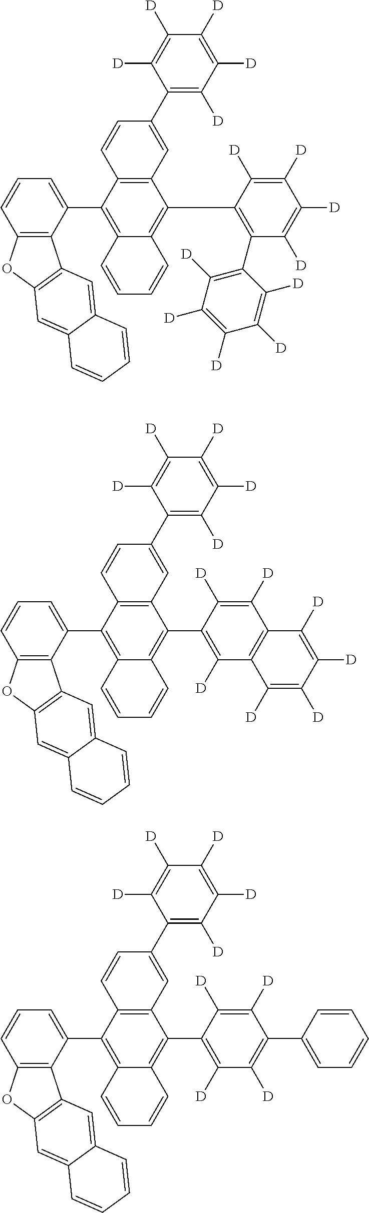 [SMILES: [2H]c1c([2H])c([2H])c(-c2ccc3c(-c4cccc5oc6cc7ccccc7cc6c45)c4ccccc4c(-c4c([2H])c([2H])c([2H])c([2H])c4-c4c([2H])c([2H])c([2H])c([2H])c4[2H])c3c2)c([2H])c1[2H] ; [2H]c1c([2H])c([2H])c(-c2ccc3c(-c4cccc5oc6cc7ccccc7cc6c45)c4ccccc4c(-c4c([2H])c([2H])c5c([2H])c([2H])c([2H])c([2H])c5c4[2H])c3c2)c([2H])c1[2H] ; [2H]c1c([2H])c([2H])c(-c2ccc3c(-c4cccc5oc6cc7ccccc7cc6c45)c4ccccc4c(-c4c([2H])c([2H])c(-c5ccccc5)c([2H])c4[2H])c3c2)c([2H])c1[2H]]

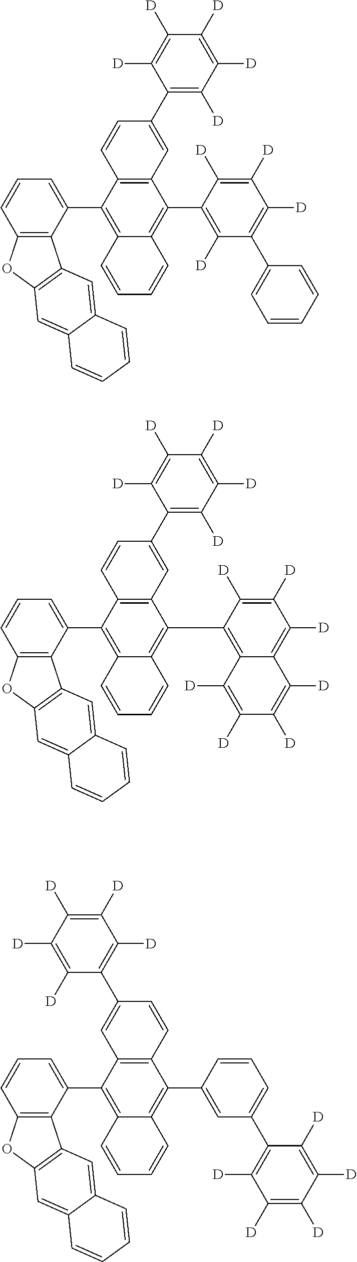 [SMILES: [2H]c1c([2H])c([2H])c(-c2ccc3c(-c4cccc5oc6cc7ccccc7cc6c45)c4ccccc4c(-c4c([2H])c([2H])c([2H])c(-c5ccccc5)c4[2H])c3c2)c([2H])c1[2H] ; [2H]c1c([2H])c([2H])c(-c2ccc3c(-c4cccc5oc6cc7ccccc7cc6c45)c4ccccc4c(-c4c([2H])c([2H])c([2H])c5c([2H])c([2H])c([2H])c([2H])c45)c3c2)c([2H])c1[2H] ; [2H]c1c([2H])c([2H])c(-c2cccc(-c3c4ccccc4c(-c4cccc5oc6cc7ccccc7cc6c45)c4cc(-c5c([2H])c([2H])c([2H])c([2H])c5[2H])ccc34)c2)c([2H])c1[2H]]

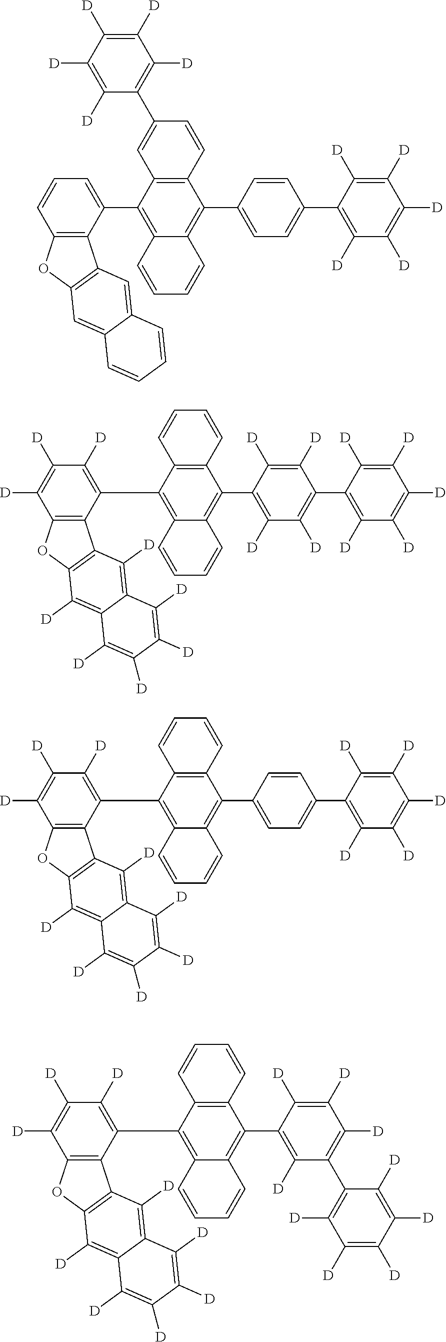

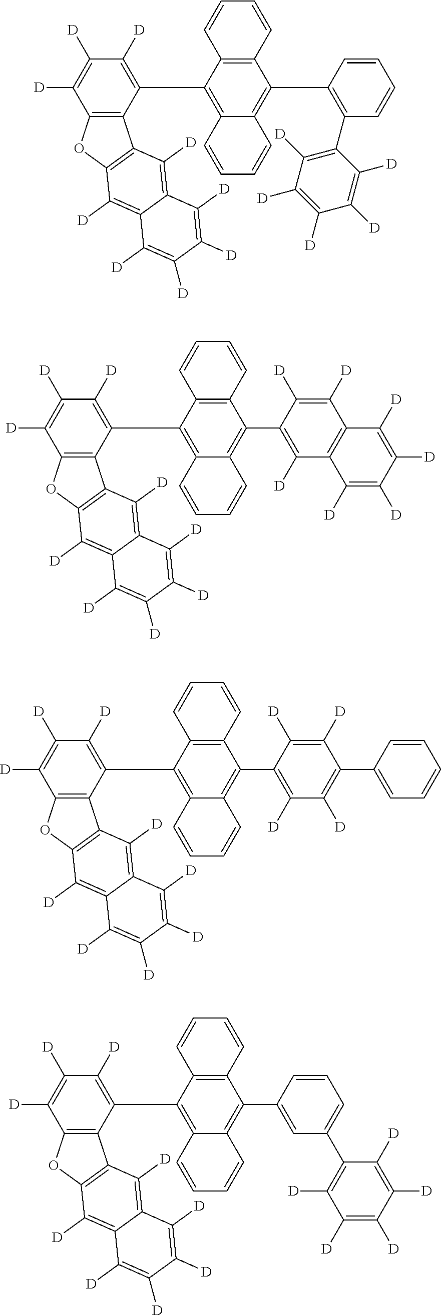

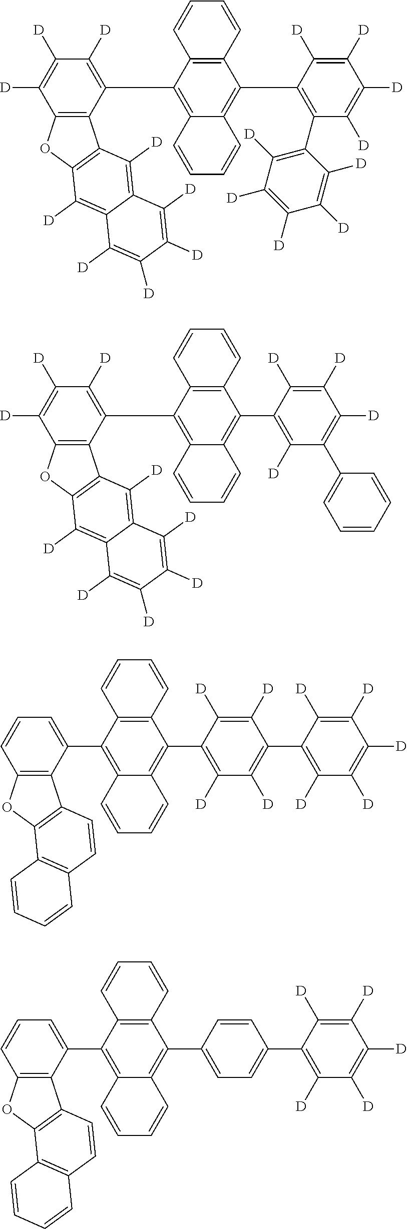

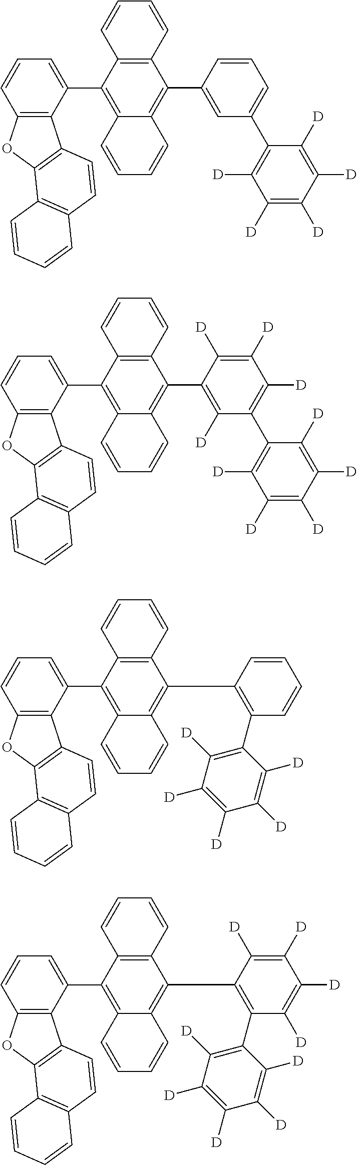 [SMILES: [2H]c1c([2H])c([2H])c(-c2cccc(-c3c4ccccc4c(-c4cccc5oc6c7ccccc7ccc6c45)c4ccccc34)c2)c([2H])c1[2H] ; [2H]c1c([2H])c([2H])c(-c2c([2H])c([2H])c([2H])c(-c3c4ccccc4c(-c4cccc5oc6c7ccccc7ccc6c45)c4ccccc34)c2[2H])c([2H])c1[2H] ; [2H]c1c([2H])c([2H])c(-c2ccccc2-c2c3ccccc3c(-c3cccc4oc5c6ccccc6ccc5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5c6ccccc6ccc5c34)c3ccccc23)c([2H])c1[2H]]

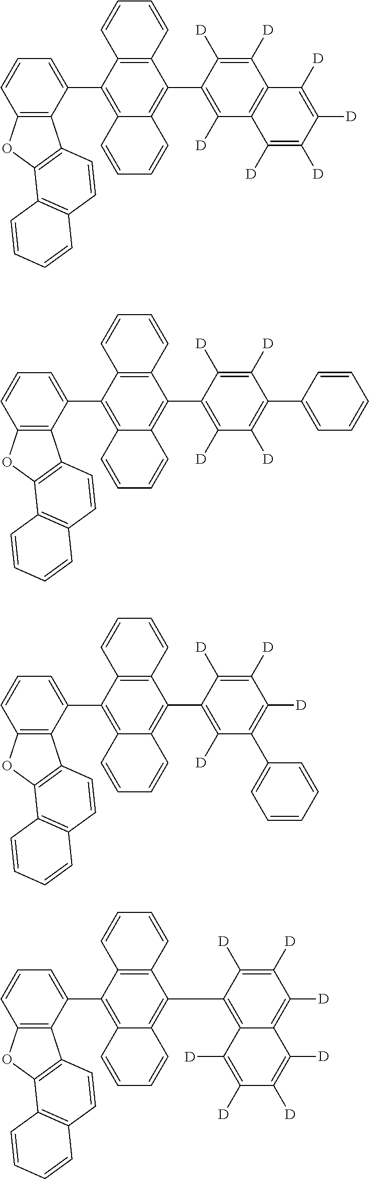 [SMILES: [2H]c1c([2H])c([2H])c2c([2H])c(-c3c4ccccc4c(-c4cccc5oc6c7ccccc7ccc6c45)c4ccccc34)c([2H])c([2H])c2c1[2H] ; [2H]c1c([2H])c(-c2c3ccccc3c(-c3cccc4oc5c6ccccc6ccc5c34)c3ccccc23)c([2H])c([2H])c1-c1ccccc1 ; [2H]c1c([2H])c(-c2ccccc2)c([2H])c(-c2c3ccccc3c(-c3cccc4oc5c6ccccc6ccc5c34)c3ccccc23)c1[2H] ; [2H]c1c([2H])c([2H])c2c(-c3c4ccccc4c(-c4cccc5oc6c7ccccc7ccc6c45)c4ccccc34)c([2H])c([2H])c([2H])c2c1[2H]]

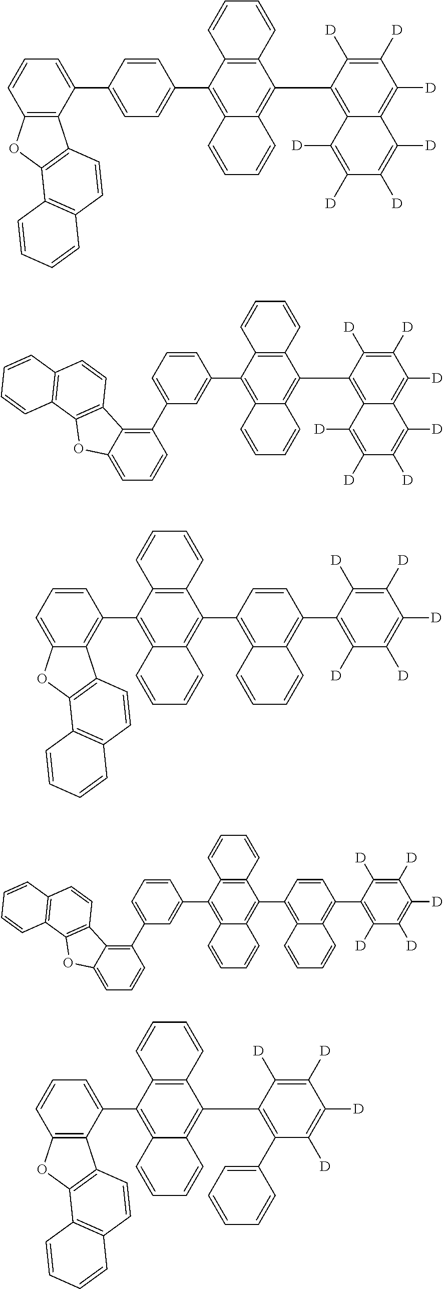

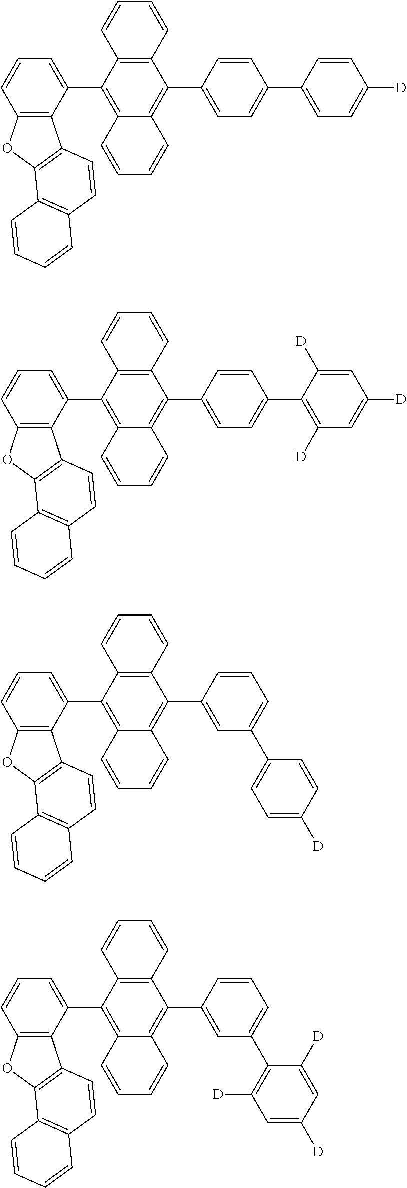 [SMILES: [2H]c1ccc(-c2ccc(-c3c4ccccc4c(-c4cccc5oc6c7ccccc7ccc6c45)c4ccccc34)cc2)cc1 ; [2H]c1cc([2H])c(-c2ccc(-c3c4ccccc4c(-c4cccc5oc6c7ccccc7ccc6c45)c4ccccc34)cc2)c([2H])c1 ; [2H]c1ccc(-c2cccc(-c3c4ccccc4c(-c4cccc5oc6c7ccccc7ccc6c45)c4ccccc34)c2)cc1 ; [2H]c1cc([2H])c(-c2cccc(-c3c4ccccc4c(-c4cccc5oc6c7ccccc7ccc6c45)c4ccccc34)c2)c([2H])c1]

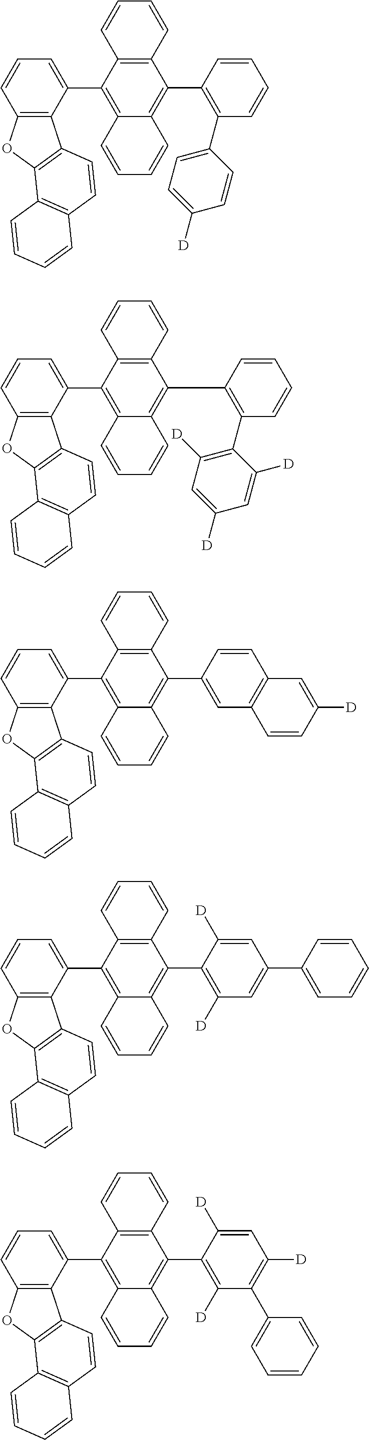 [SMILES: [2H]c1ccc(-c2ccccc2-c2c3ccccc3c(-c3cccc4oc5c6ccccc6ccc5c34)c3ccccc23)cc1 ; [2H]c1cc([2H])c(-c2ccccc2-c2c3ccccc3c(-c3cccc4oc5c6ccccc6ccc5c34)c3ccccc23)c([2H])c1 ; [2H]c1ccc2cc(-c3c4ccccc4c(-c4cccc5oc6c7ccccc7ccc6c45)c4ccccc34)ccc2c1 ; [2H]c1cc(-c2ccccc2)cc([2H])c1-c1c2ccccc2c(-c2cccc3oc4c5ccccc5ccc4c23)c2ccccc12 ; [2H]c1cc([2H])c(-c2c3ccccc3c(-c3cccc4oc5c6ccccc6ccc5c34)c3ccccc23)c([2H])c1-c1ccccc1]

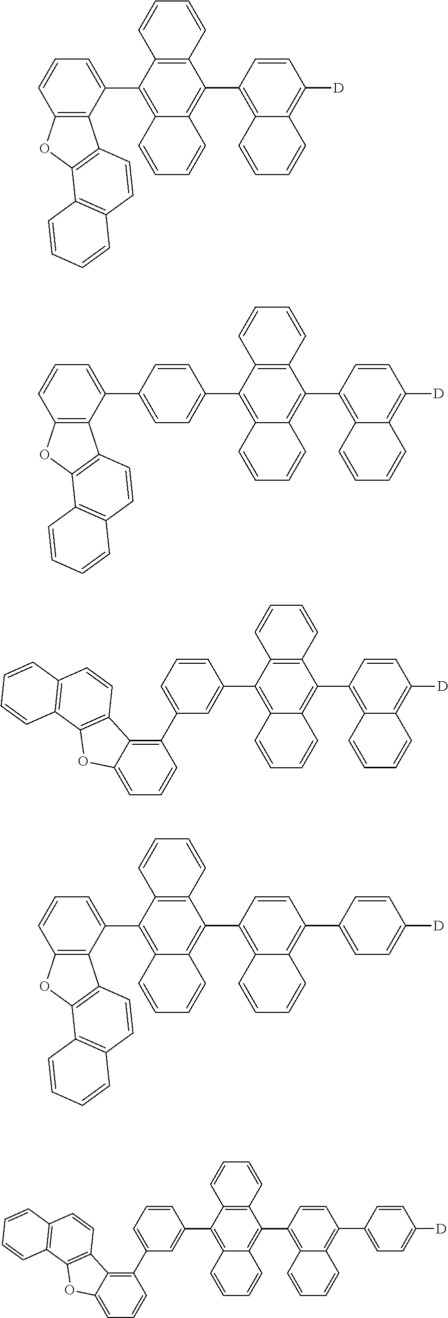

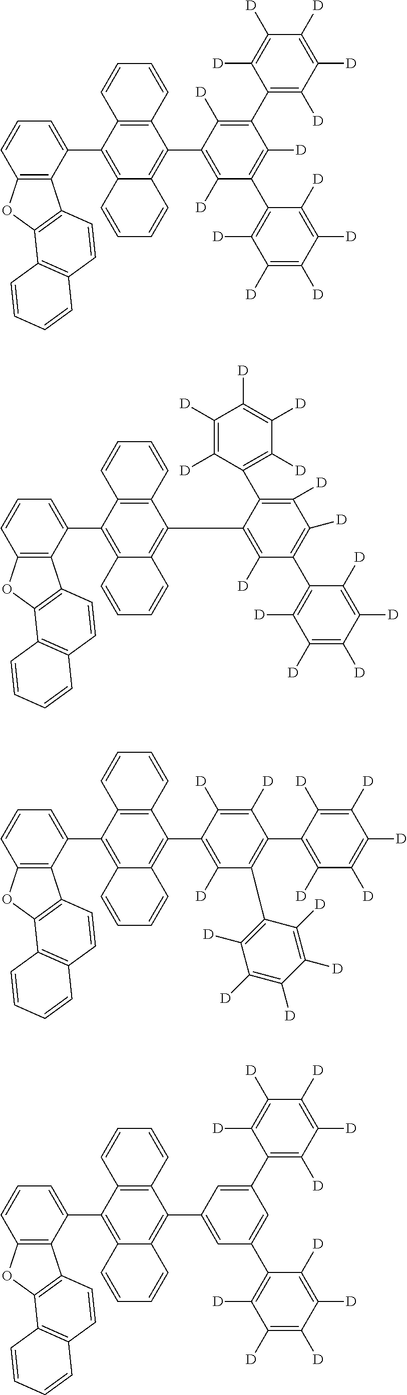

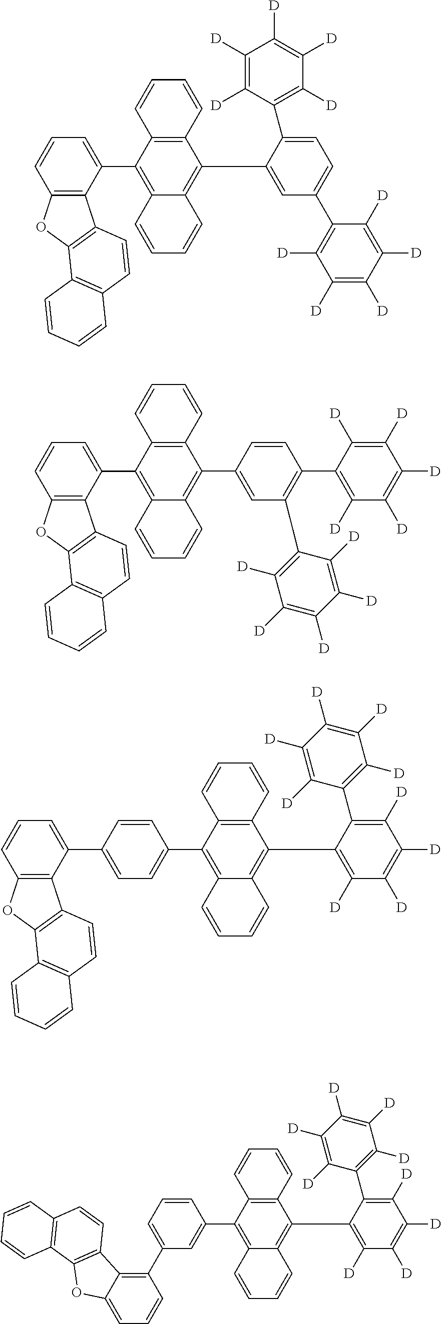

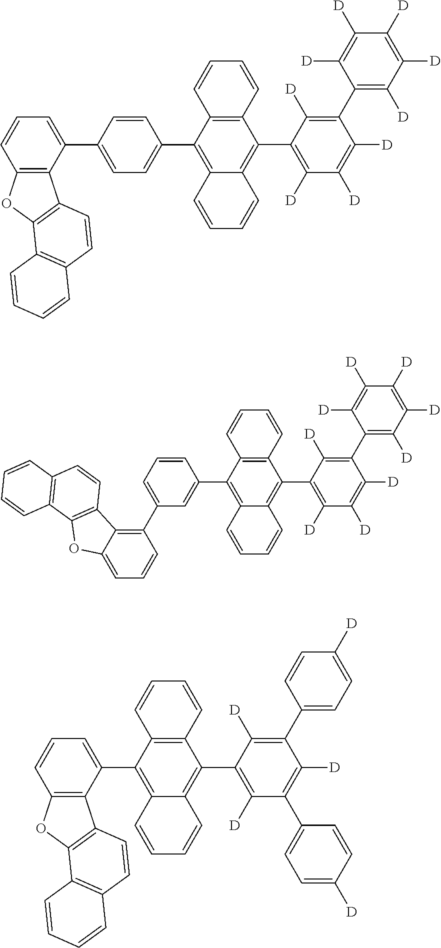 [SMILES: [2H]c1c([2H])c([2H])c(-c2c([2H])c([2H])c([2H])c(-c3c4ccccc4c(-c4ccc(-c5cccc6oc7c8ccccc8ccc7c56)cc4)c4ccccc34)c2[2H])c([2H])c1[2H] ; [2H]c1c([2H])c([2H])c(-c2c([2H])c([2H])c([2H])c(-c3c4ccccc4c(-c4cccc(-c5cccc6oc7c8ccccc8ccc7c56)c4)c4ccccc34)c2[2H])c([2H])c1[2H] ; [2H]c1ccc(-c2c([2H])c(-c3ccc([2H])cc3)c([2H])c(-c3c4ccccc4c(-c4cccc5oc6c7ccccc7ccc6c45)c4ccccc34)c2[2H])cc1]

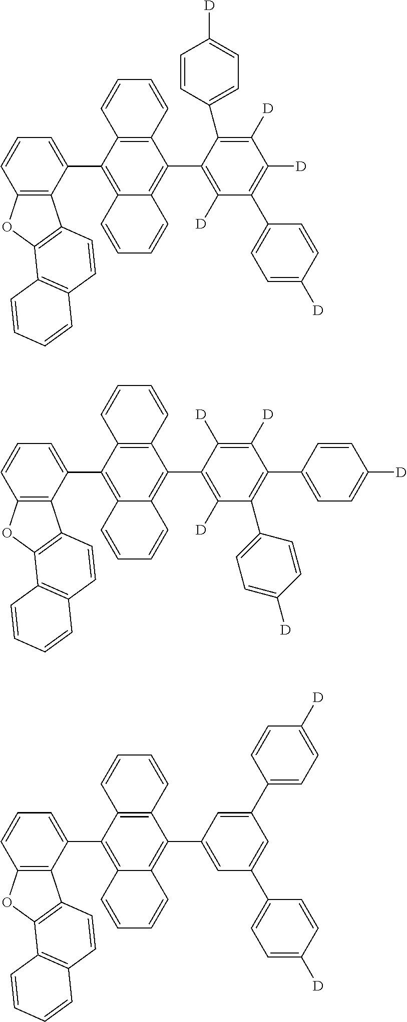 [SMILES: [2H]c1ccc(-c2c([2H])c([2H])c(-c3ccc([2H])cc3)c(-c3c4ccccc4c(-c4cccc5oc6c7ccccc7ccc6c45)c4ccccc34)c2[2H])cc1 ; [2H]c1ccc(-c2c([2H])c([2H])c(-c3c4ccccc4c(-c4cccc5oc6c7ccccc7ccc6c45)c4ccccc34)c([2H])c2-c2ccc([2H])cc2)cc1 ; [2H]c1ccc(-c2cc(-c3ccc([2H])cc3)cc(-c3c4ccccc4c(-c4cccc5oc6c7ccccc7ccc6c45)c4ccccc34)c2)cc1]

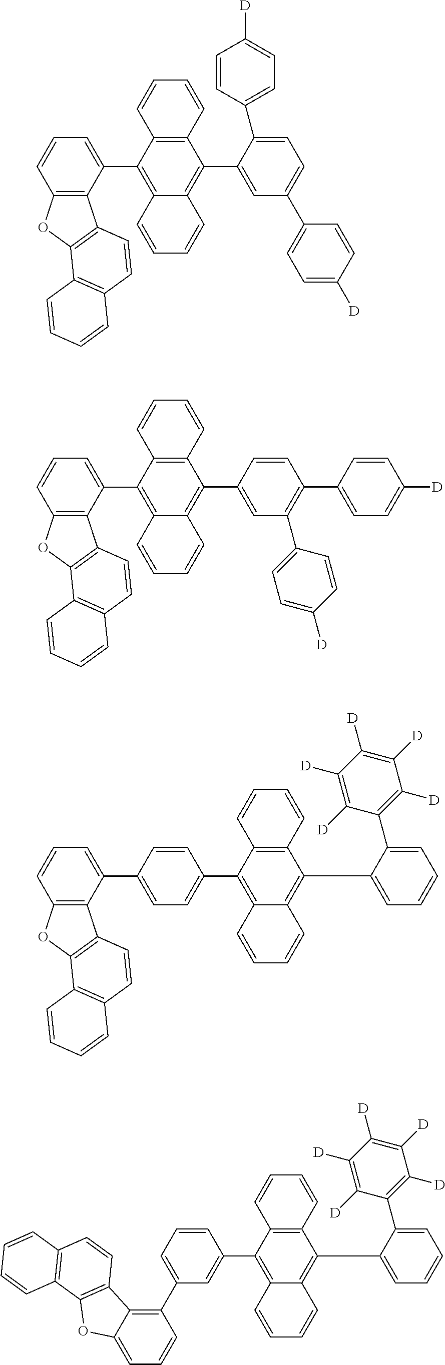 [SMILES: [2H]c1ccc(-c2ccc(-c3ccc([2H])cc3)c(-c3c4ccccc4c(-c4cccc5oc6c7ccccc7ccc6c45)c4ccccc34)c2)cc1 ; [2H]c1ccc(-c2ccc(-c3c4ccccc4c(-c4cccc5oc6c7ccccc7ccc6c45)c4ccccc34)cc2-c2ccc([2H])cc2)cc1 ; [2H]c1c([2H])c([2H])c(-c2ccccc2-c2c3ccccc3c(-c3ccc(-c4cccc5oc6c7ccccc7ccc6c45)cc3)c3ccccc23)c([2H])c1[2H] ; [2H]c1c([2H])c([2H])c(-c2ccccc2-c2c3ccccc3c(-c3cccc(-c4cccc5oc6c7ccccc7ccc6c45)c3)c3ccccc23)c([2H])c1[2H]]

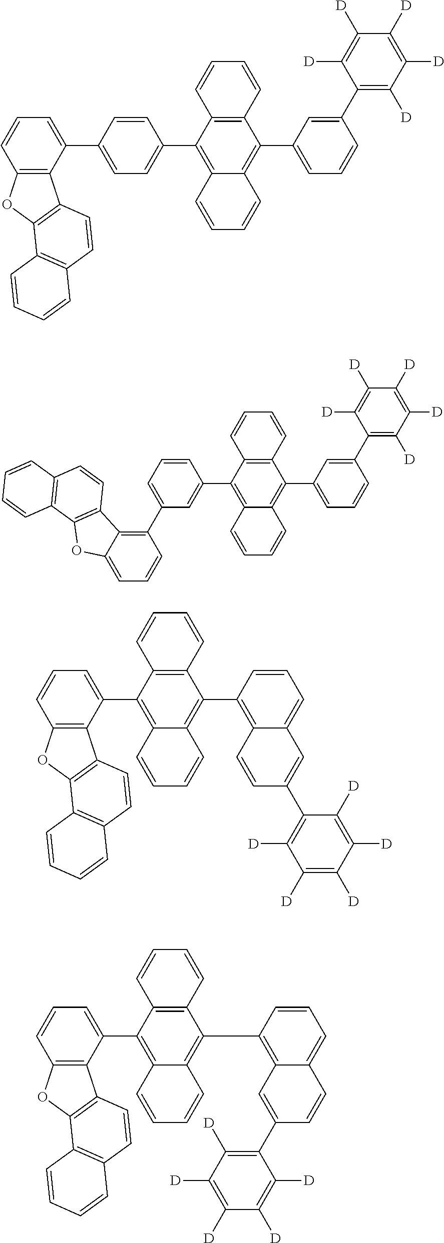

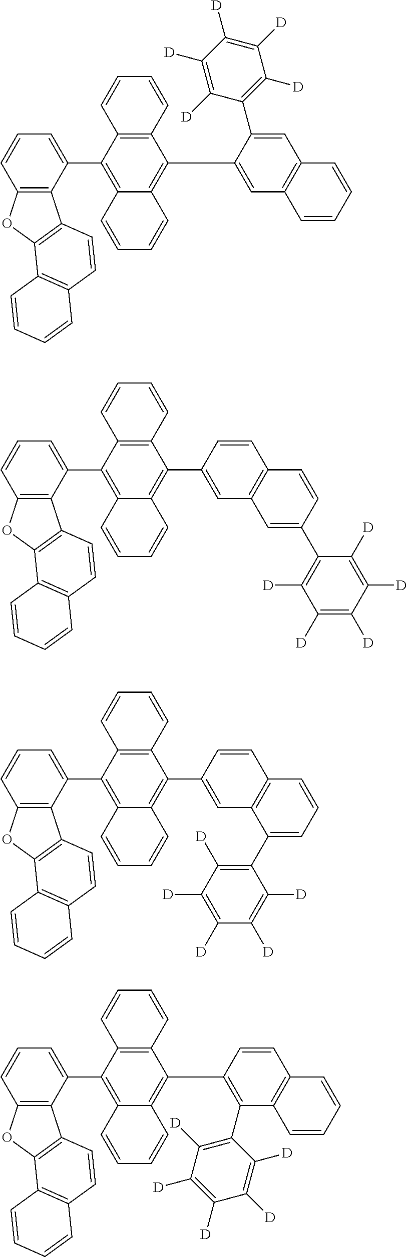 [SMILES: [2H]c1c([2H])c([2H])c(-c2cc3ccccc3cc2-c2c3ccccc3c(-c3cccc4oc5c6ccccc6ccc5c34)c3ccccc23)c([2H])c1[2H] ; [2H]c1c([2H])c([2H])c(-c2ccc3ccc(-c4c5ccccc5c(-c5cccc6oc7c8ccccc8ccc7c56)c5ccccc45)cc3c2)c([2H])c1[2H] ; [2H]c1c([2H])c([2H])c(-c2cccc3ccc(-c4c5ccccc5c(-c5cccc6oc7c8ccccc8ccc7c56)c5ccccc45)cc23)c([2H])c1[2H] ; [2H]c1c([2H])c([2H])c(-c2c(-c3c4ccccc4c(-c4cccc5oc6c7ccccc7ccc6c45)c4ccccc34)ccc3ccccc23)c([2H])c1[2H]]

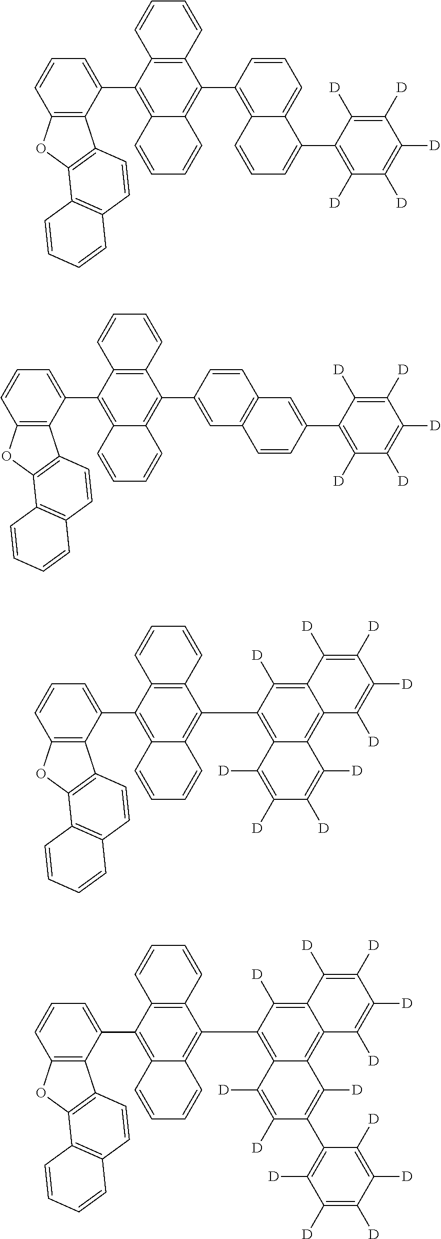 [SMILES: [2H]c1c([2H])c([2H])c(-c2cccc3c(-c4c5ccccc5c(-c5cccc6oc7c8ccccc8ccc7c56)c5ccccc45)cccc23)c([2H])c1[2H] ; [2H]c1c([2H])c([2H])c(-c2ccc3cc(-c4c5ccccc5c(-c5cccc6oc7c8ccccc8ccc7c56)c5ccccc45)ccc3c2)c([2H])c1[2H] ; [2H]c1c([2H])c([2H])c2c(c1[2H])c([2H])c(-c1c3ccccc3c(-c3cccc4oc5c6ccccc6ccc5c34)c3ccccc13)c1c([2H])c([2H])c([2H])c([2H])c12 ; [2H]c1c([2H])c([2H])c(-c2c([2H])c([2H])c3c(-c4c5ccccc5c(-c5cccc6oc7c8ccccc8ccc7c56)c5ccccc45)c([2H])c4c([2H])c([2H])c([2H])c([2H])c4c3c2[2H])c([2H])c1[2H]]

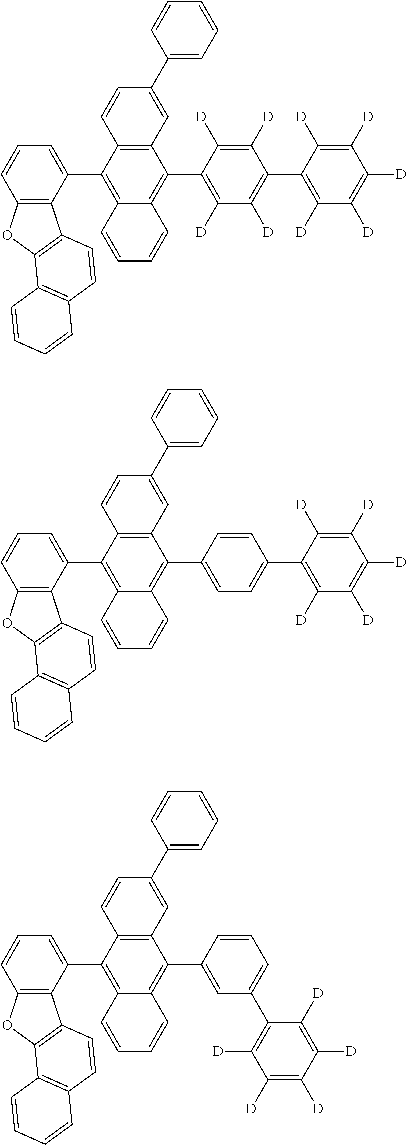 [SMILES: [2H]c1c([2H])c([2H])c(-c2c([2H])c([2H])c(-c3c4ccccc4c(-c4cccc5oc6c7ccccc7ccc6c45)c4ccc(-c5ccccc5)cc34)c([2H])c2[2H])c([2H])c1[2H] ; [2H]c1c([2H])c([2H])c(-c2ccc(-c3c4ccccc4c(-c4cccc5oc6c7ccccc7ccc6c45)c4ccc(-c5ccccc5)cc34)cc2)c([2H])c1[2H] ; [2H]c1c([2H])c([2H])c(-c2cccc(-c3c4ccccc4c(-c4cccc5oc6c7ccccc7ccc6c45)c4ccc(-c5ccccc5)cc34)c2)c([2H])c1[2H]]

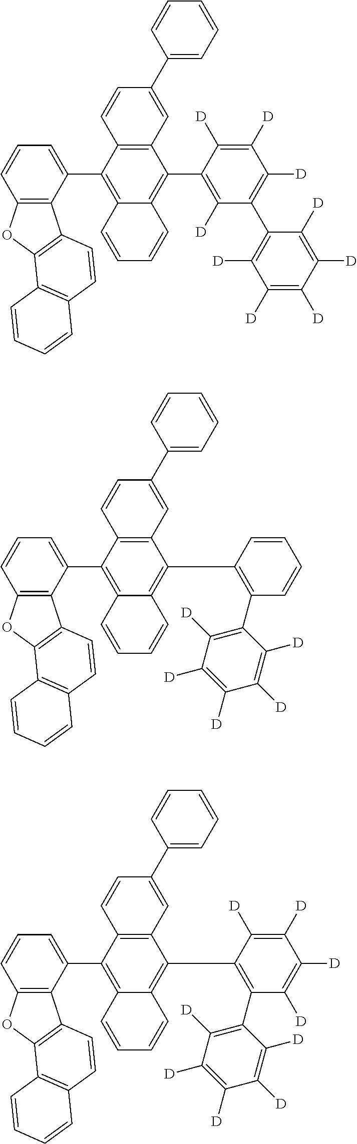 [SMILES: [2H]c1c([2H])c([2H])c(-c2c([2H])c([2H])c([2H])c(-c3c4ccccc4c(-c4cccc5oc6c7ccccc7ccc6c45)c4ccc(-c5ccccc5)cc34)c2[2H])c([2H])c1[2H] ; [2H]c1c([2H])c([2H])c(-c2ccccc2-c2c3ccccc3c(-c3cccc4oc5c6ccccc6ccc5c34)c3ccc(-c4ccccc4)cc23)c([2H])c1[2H] ; [2H]c1c([2H])c([2H])c(-c2c([2H])c([2H])c([2H])c([2H])c2-c2c3ccccc3c(-c3cccc4oc5c6ccccc6ccc5c34)c3ccc(-c4ccccc4)cc23)c([2H])c1[2H]]

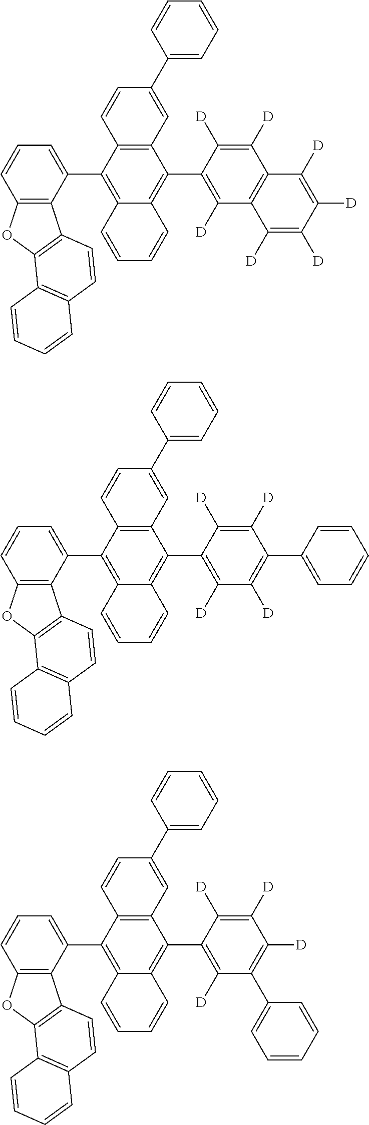 [SMILES: [2H]c1c([2H])c([2H])c2c([2H])c(-c3c4ccccc4c(-c4cccc5oc6c7ccccc7ccc6c45)c4ccc(-c5ccccc5)cc34)c([2H])c([2H])c2c1[2H] ; [2H]c1c([2H])c(-c2c3ccccc3c(-c3cccc4oc5c6ccccc6ccc5c34)c3ccc(-c4ccccc4)cc23)c([2H])c([2H])c1-c1ccccc1 ; [2H]c1c([2H])c(-c2ccccc2)c([2H])c(-c2c3ccccc3c(-c3cccc4oc5c6ccccc6ccc5c34)c3ccc(-c4ccccc4)cc23)c1[2H]]

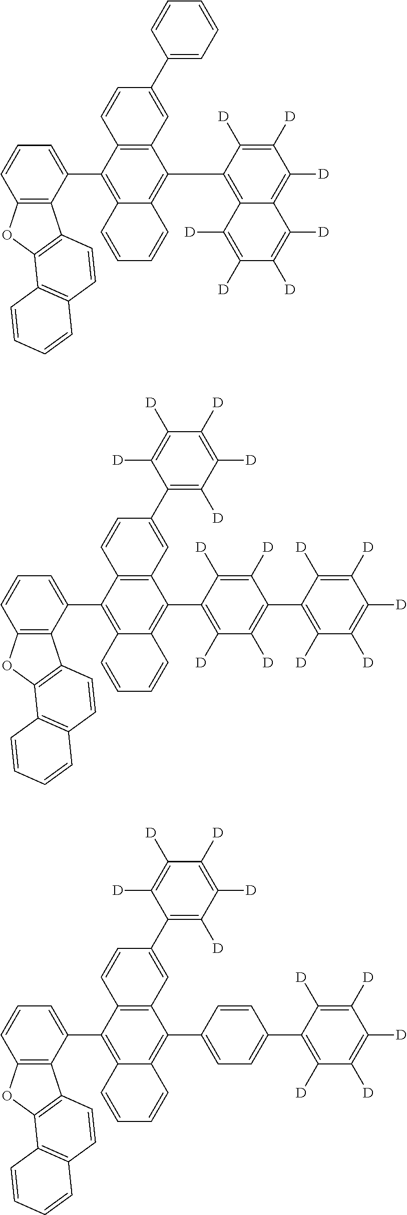 [SMILES: [2H]c1c([2H])c([2H])c2c(-c3c4ccccc4c(-c4cccc5oc6c7ccccc7ccc6c45)c4ccc(-c5ccccc5)cc34)c([2H])c([2H])c([2H])c2c1[2H] ; [2H]c1c([2H])c([2H])c(-c2ccc3c(-c4cccc5oc6c7ccccc7ccc6c45)c4ccccc4c(-c4c([2H])c([2H])c(-c5c([2H])c([2H])c([2H])c([2H])c5[2H])c([2H])c4[2H])c3c2)c([2H])c1[2H] ; [2H]c1c([2H])c([2H])c(-c2ccc(-c3c4ccccc4c(-c4cccc5oc6c7ccccc7ccc6c45)c4ccc(-c5c([2H])c([2H])c([2H])c([2H])c5[2H])cc34)cc2)c([2H])c1[2H]]

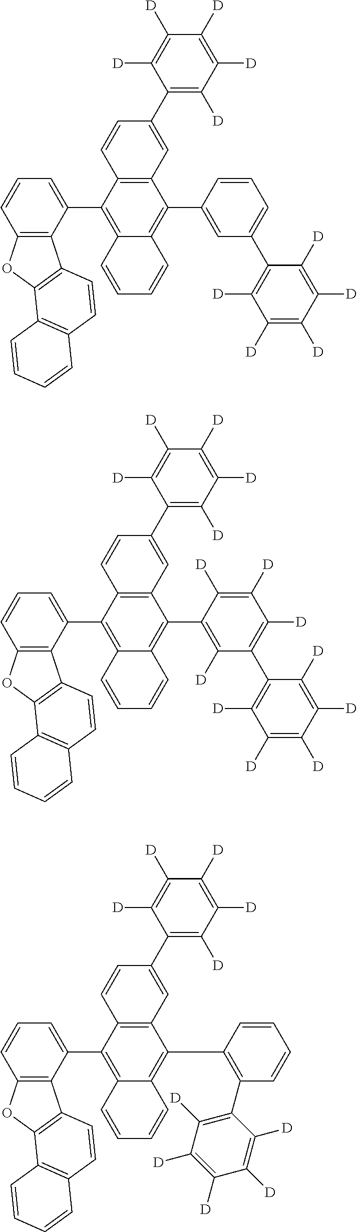 [SMILES: [2H]c1c([2H])c([2H])c(-c2cccc(-c3c4ccccc4c(-c4cccc5oc6c7ccccc7ccc6c45)c4ccc(-c5c([2H])c([2H])c([2H])c([2H])c5[2H])cc34)c2)c([2H])c1[2H] ; [2H]c1c([2H])c([2H])c(-c2ccc3c(-c4cccc5oc6c7ccccc7ccc6c45)c4ccccc4c(-c4c([2H])c([2H])c([2H])c(-c5c([2H])c([2H])c([2H])c([2H])c5[2H])c4[2H])c3c2)c([2H])c1[2H] ; [2H]c1c([2H])c([2H])c(-c2ccc3c(-c4cccc5oc6c7ccccc7ccc6c45)c4ccccc4c(-c4ccccc4-c4c([2H])c([2H])c([2H])c([2H])c4[2H])c3c2)c([2H])c1[2H]]

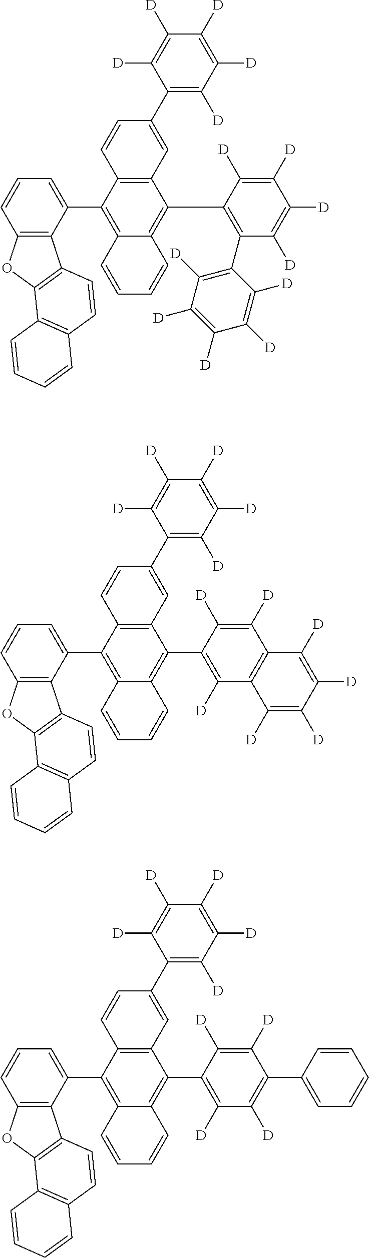 [SMILES: [2H]c1c([2H])c([2H])c(-c2ccc3c(-c4cccc5oc6c7ccccc7ccc6c45)c4ccccc4c(-c4c([2H])c([2H])c([2H])c([2H])c4-c4c([2H])c([2H])c([2H])c([2H])c4[2H])c3c2)c([2H])c1[2H] ; [2H]c1c([2H])c([2H])c(-c2ccc3c(-c4cccc5oc6c7ccccc7ccc6c45)c4ccccc4c(-c4c([2H])c([2H])c5c([2H])c([2H])c([2H])c([2H])c5c4[2H])c3c2)c([2H])c1[2H] ; [2H]c1c([2H])c([2H])c(-c2ccc3c(-c4cccc5oc6c7ccccc7ccc6c45)c4ccccc4c(-c4c([2H])c([2H])c(-c5ccccc5)c([2H])c4[2H])c3c2)c([2H])c1[2H]]

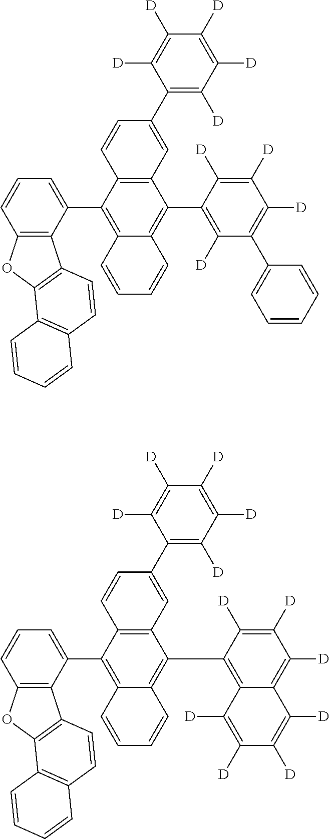 [SMILES: [2H]c1c([2H])c([2H])c(-c2ccc3c(-c4cccc5oc6c7ccccc7ccc6c45)c4ccccc4c(-c4c([2H])c([2H])c([2H])c(-c5ccccc5)c4[2H])c3c2)c([2H])c1[2H] ; [2H]c1c([2H])c([2H])c(-c2ccc3c(-c4cccc5oc6c7ccccc7ccc6c45)c4ccccc4c(-c4c([2H])c([2H])c([2H])c5c([2H])c([2H])c([2H])c([2H])c45)c3c2)c([2H])c1[2H]]

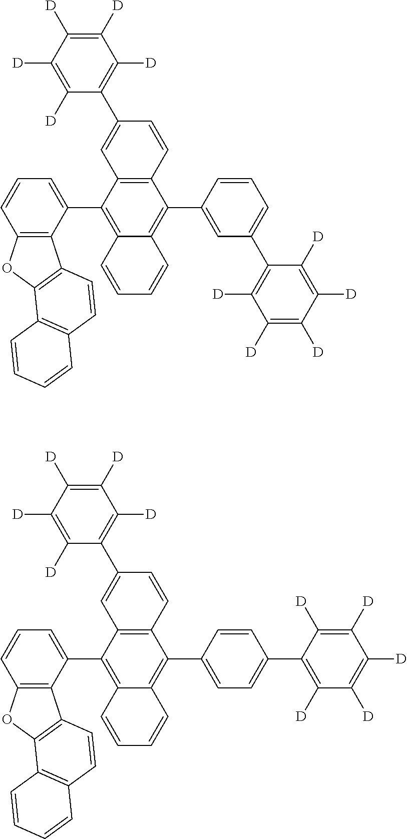 [SMILES: [2H]c1c([2H])c([2H])c(-c2cccc(-c3c4ccccc4c(-c4cccc5oc6c7ccccc7ccc6c45)c4cc(-c5c([2H])c([2H])c([2H])c([2H])c5[2H])ccc34)c2)c([2H])c1[2H] ; [2H]c1c([2H])c([2H])c(-c2ccc(-c3c4ccccc4c(-c4cccc5oc6c7ccccc7ccc6c45)c4cc(-c5c([2H])c([2H])c([2H])c([2H])c5[2H])ccc34)cc2)c([2H])c1[2H]]

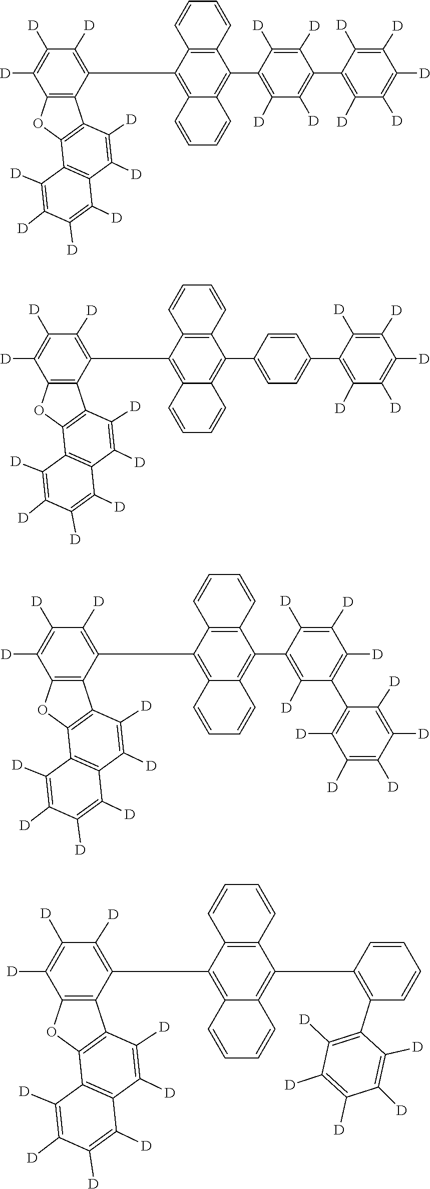

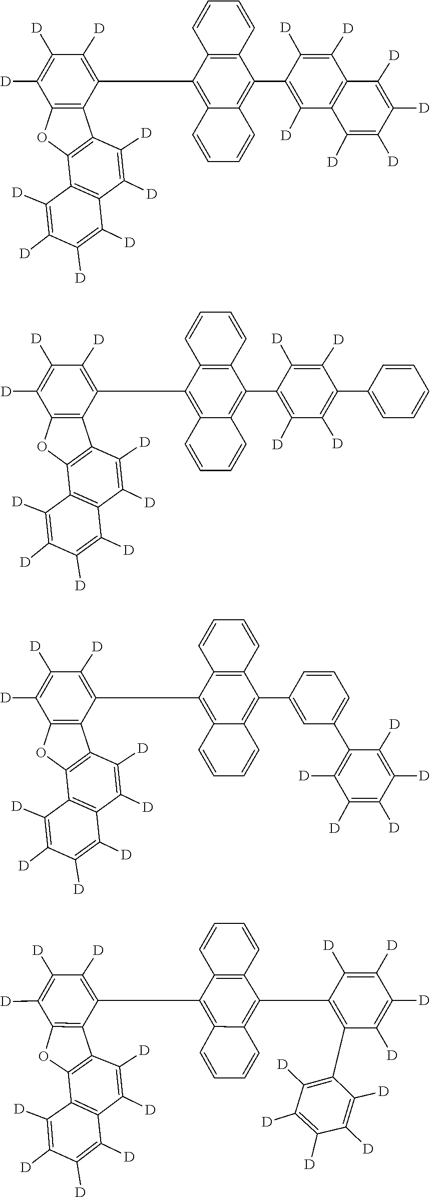

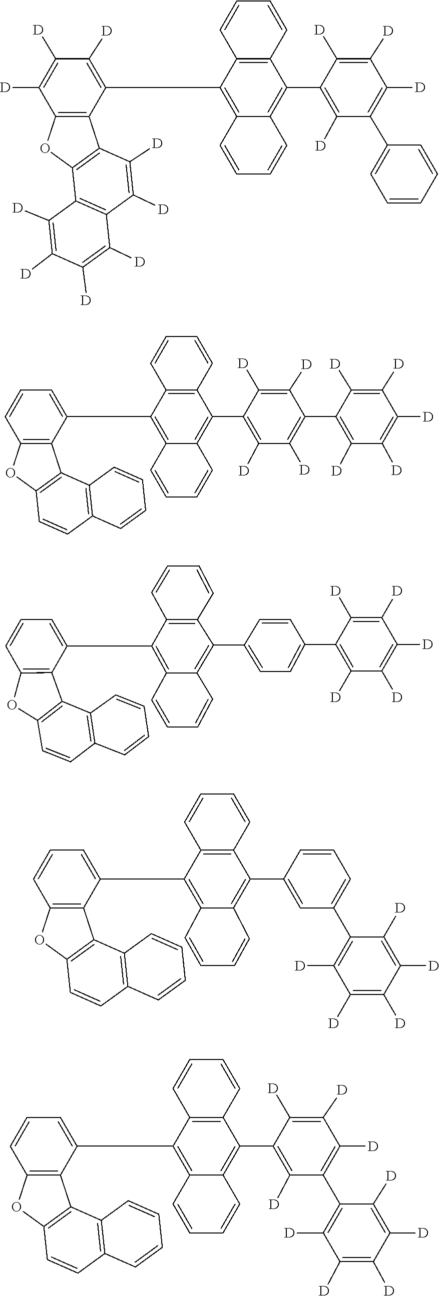

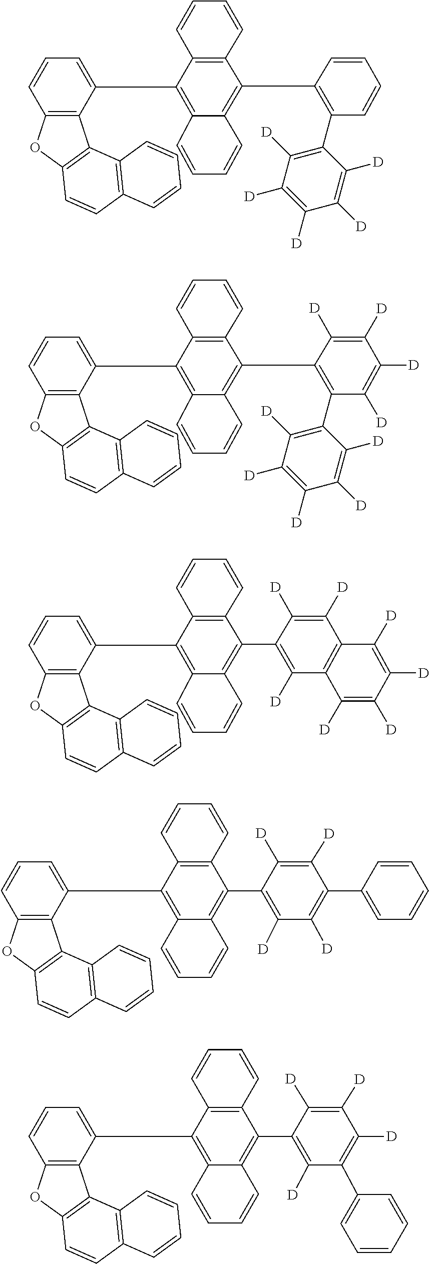

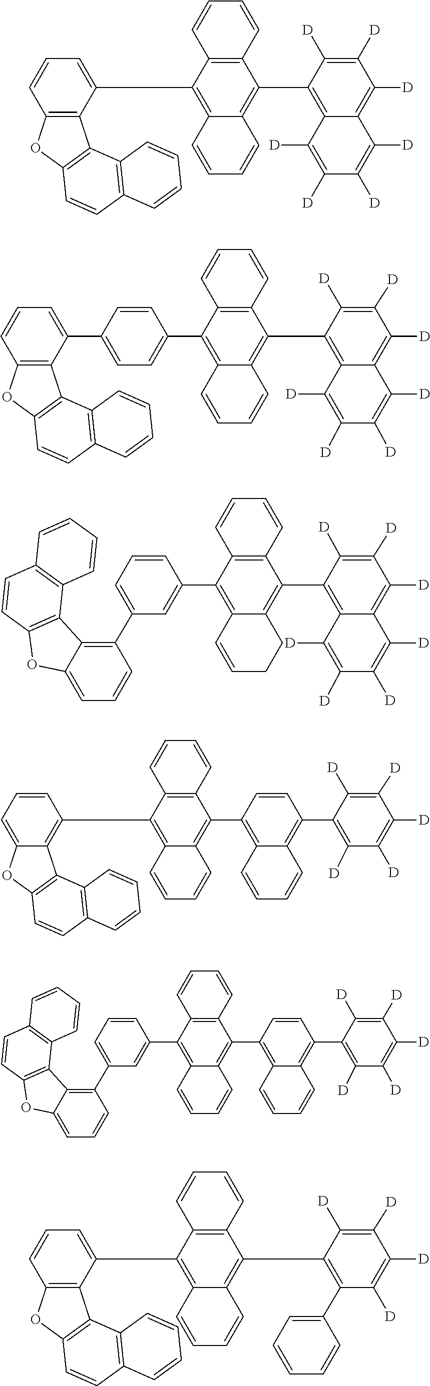

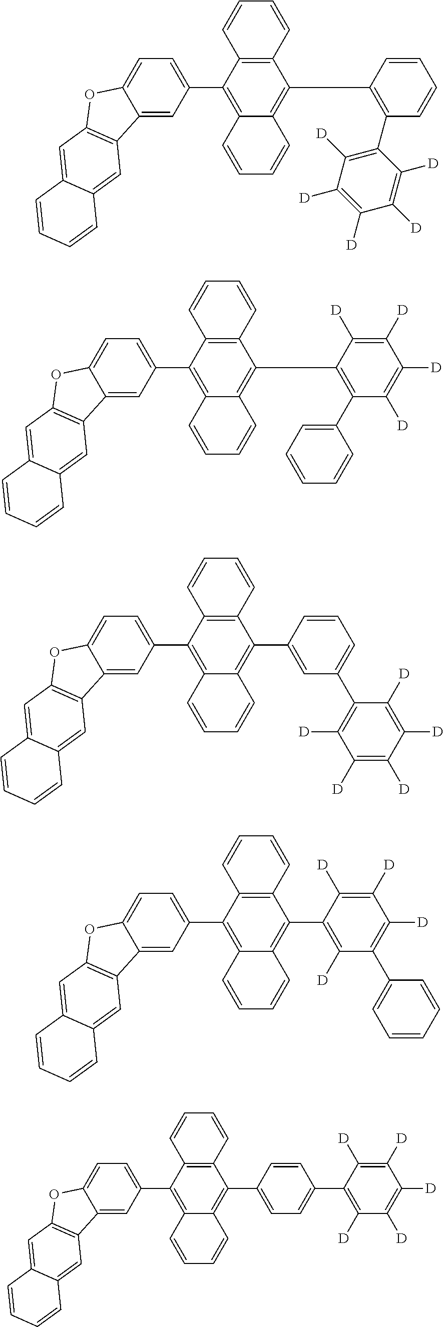 [SMILES: [2H]c1c([2H])c([2H])c(-c2ccccc2-c2c3ccccc3c(-c3ccc4oc5cc6ccccc6cc5c4c3)c3ccccc23)c([2H])c1[2H] ; [2H]c1c([2H])c([2H])c(-c2c3ccccc3c(-c3ccc4oc5cc6ccccc6cc5c4c3)c3ccccc23)c(-c2ccccc2)c1[2H] ; [2H]c1c([2H])c([2H])c(-c2cccc(-c3c4ccccc4c(-c4ccc5oc6cc7ccccc7cc6c5c4)c4ccccc34)c2)c([2H])c1[2H] ; [2H]c1c([2H])c(-c2ccccc2)c([2H])c(-c2c3ccccc3c(-c3ccc4oc5cc6ccccc6cc5c4c3)c3ccccc23)c1[2H] ; [2H]c1c([2H])c([2H])c(-c2ccc(-c3c4ccccc4c(-c4ccc5oc6cc7ccccc7cc6c5c4)c4ccccc34)cc2)c([2H])c1[2H]]

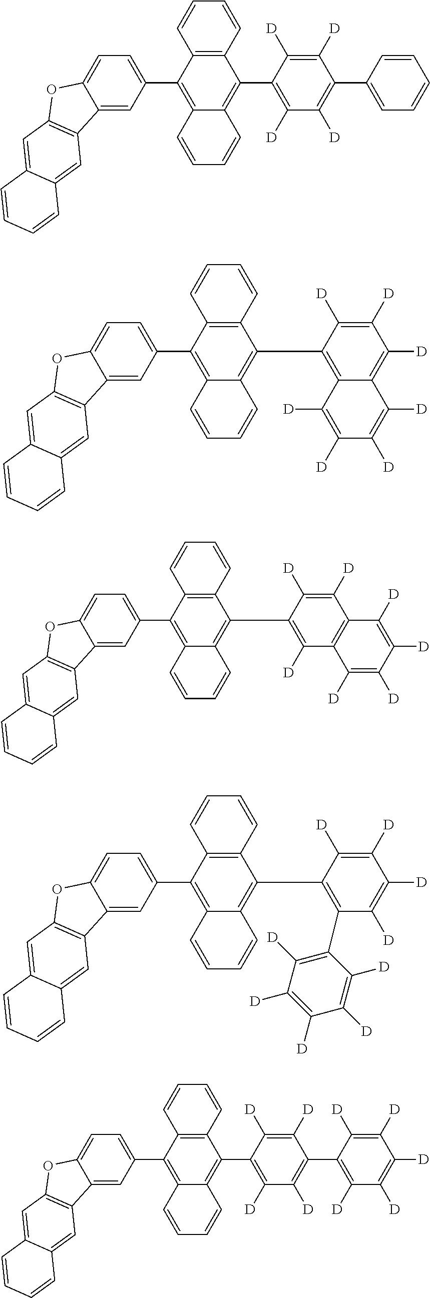

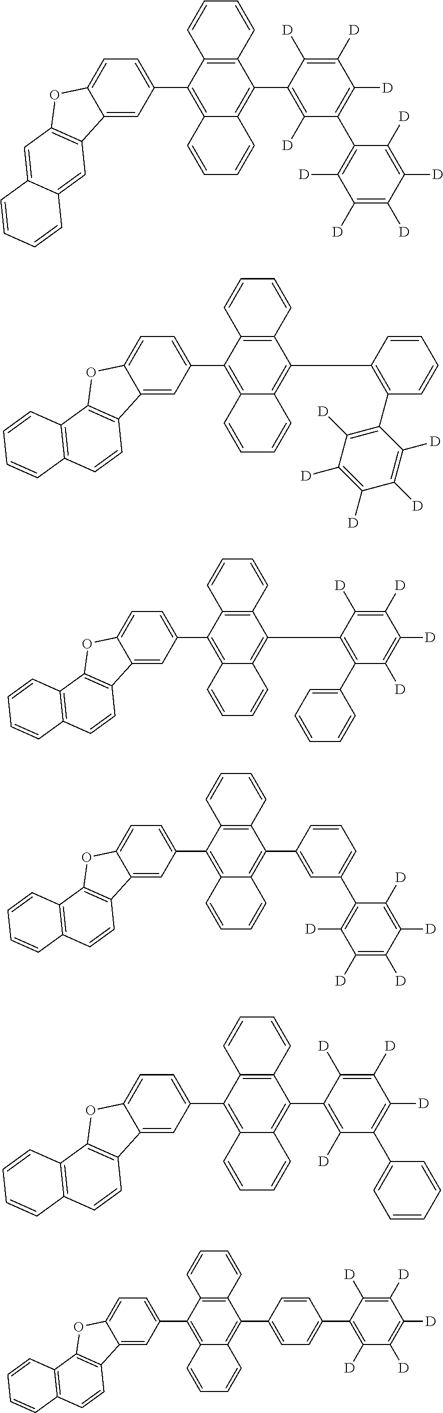

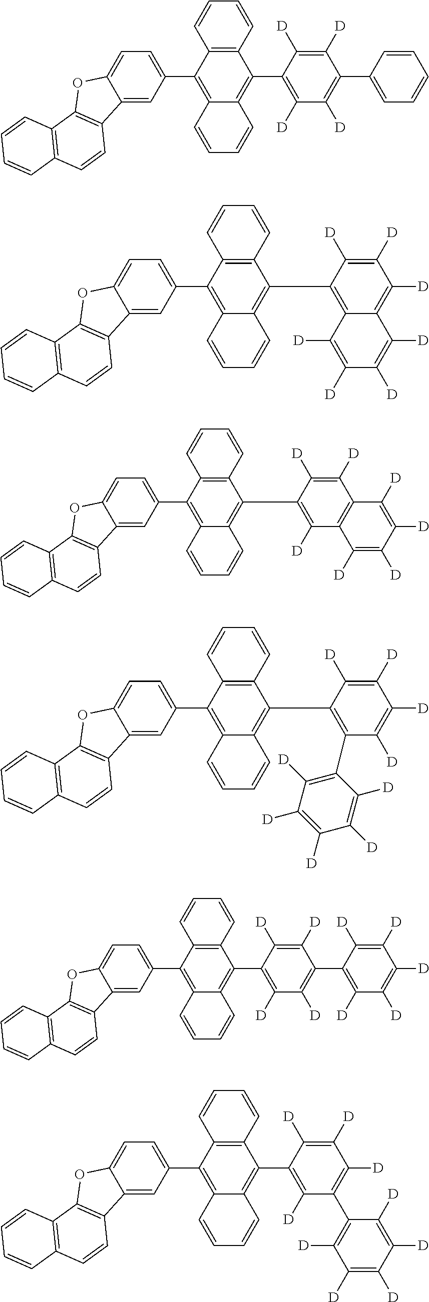

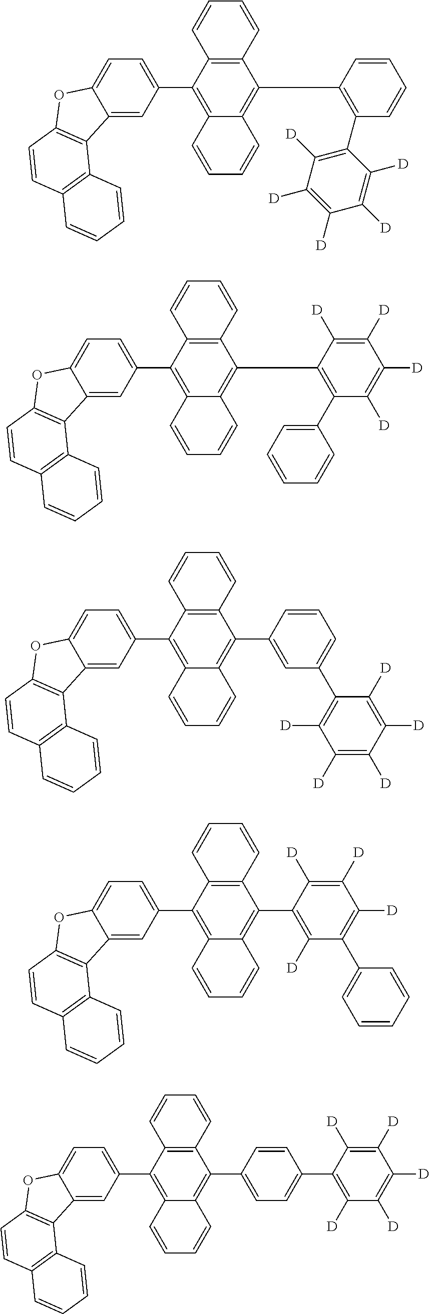 [SMILES: [2H]c1c([2H])c([2H])c(-c2ccccc2-c2c3ccccc3c(-c3ccc4oc5ccc6ccccc6c5c4c3)c3ccccc23)c([2H])c1[2H] ; [2H]c1c([2H])c([2H])c(-c2c3ccccc3c(-c3ccc4oc5ccc6ccccc6c5c4c3)c3ccccc23)c(-c2ccccc2)c1[2H] ; [2H]c1c([2H])c([2H])c(-c2cccc(-c3c4ccccc4c(-c4ccc5oc6ccc7ccccc7c6c5c4)c4ccccc34)c2)c([2H])c1[2H] ; [2H]c1c([2H])c(-c2ccccc2)c([2H])c(-c2c3ccccc3c(-c3ccc4oc5ccc6ccccc6c5c4c3)c3ccccc23)c1[2H] ; [2H]c1c([2H])c([2H])c(-c2ccc(-c3c4ccccc4c(-c4ccc5oc6ccc7ccccc7c6c5c4)c4ccccc34)cc2)c([2H])c1[2H]]

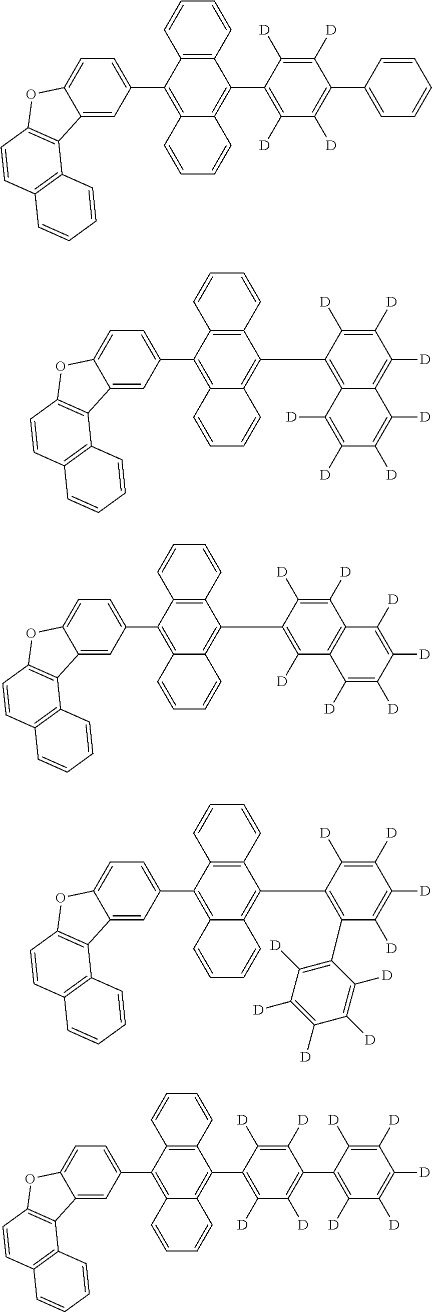

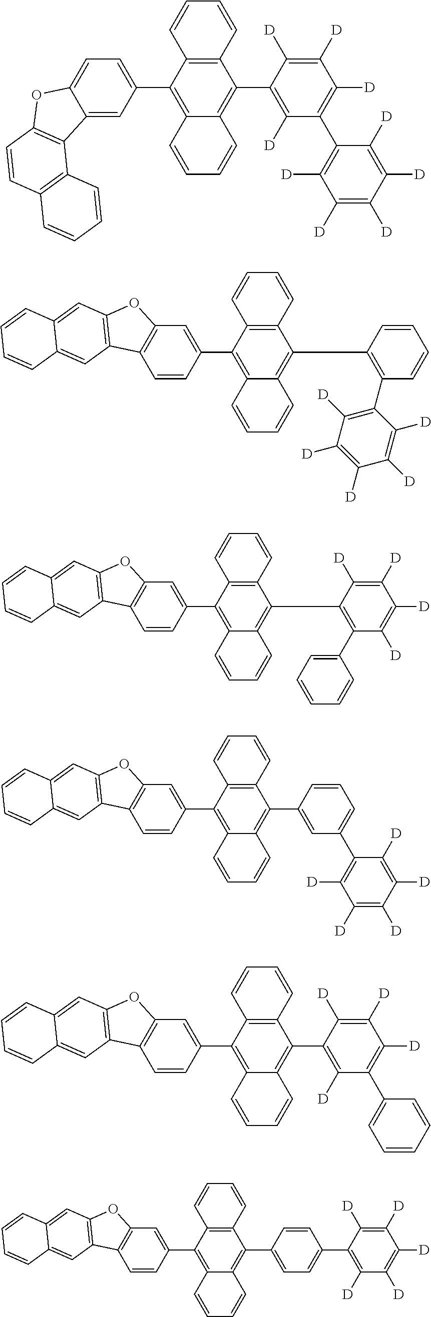

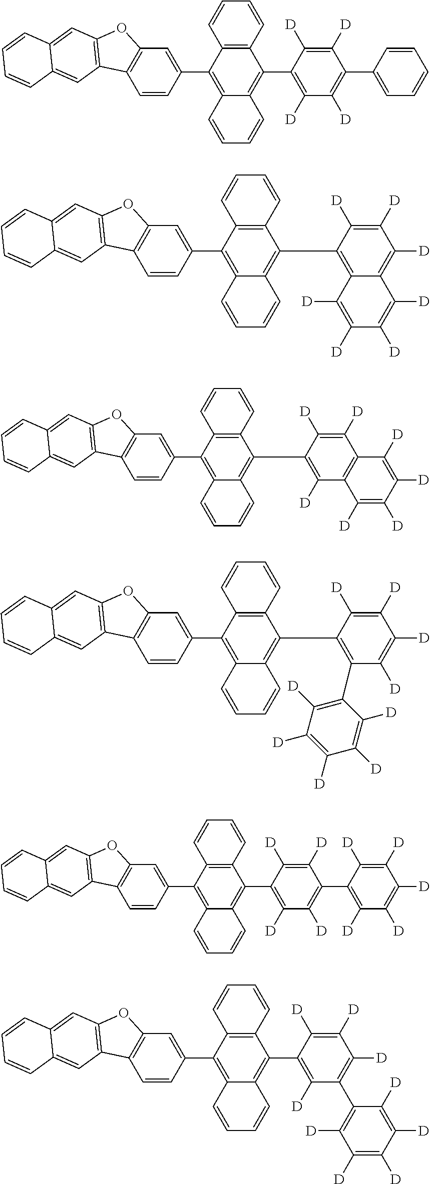

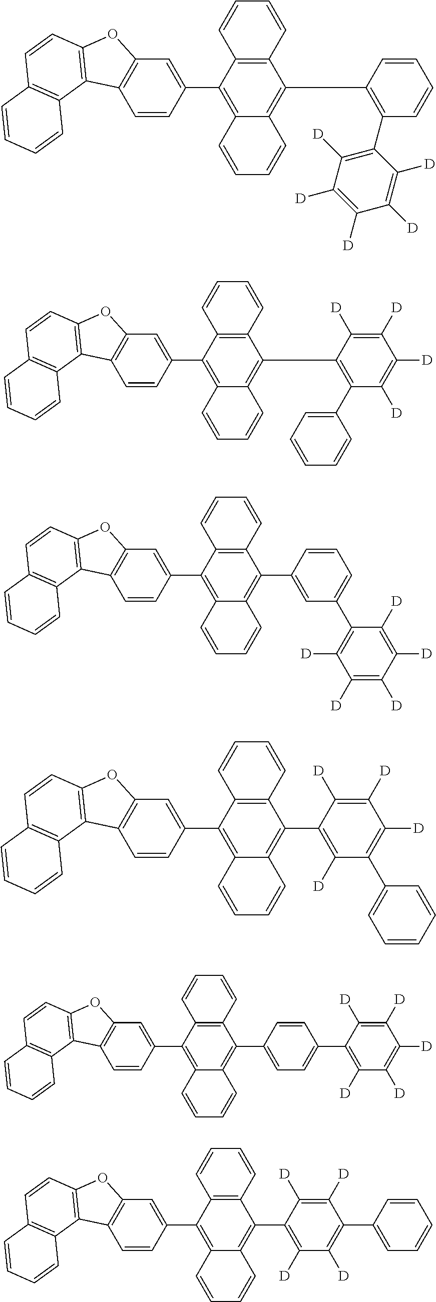

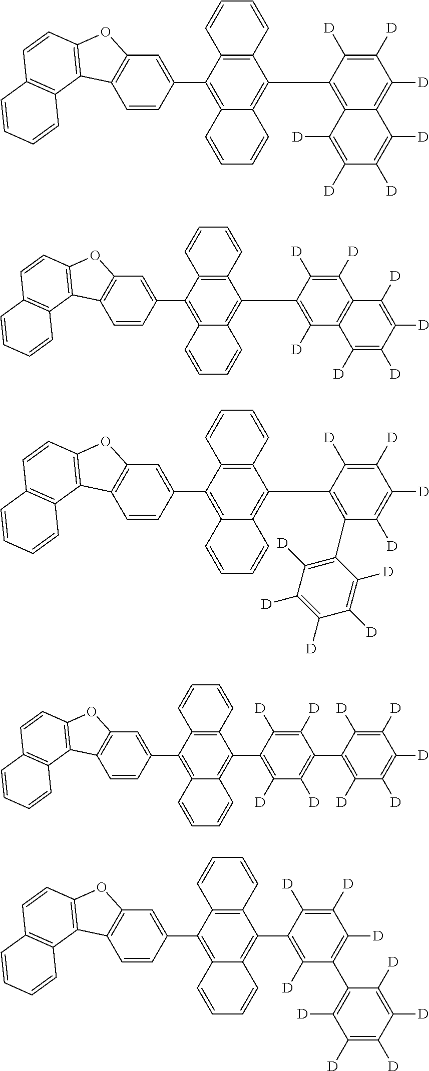

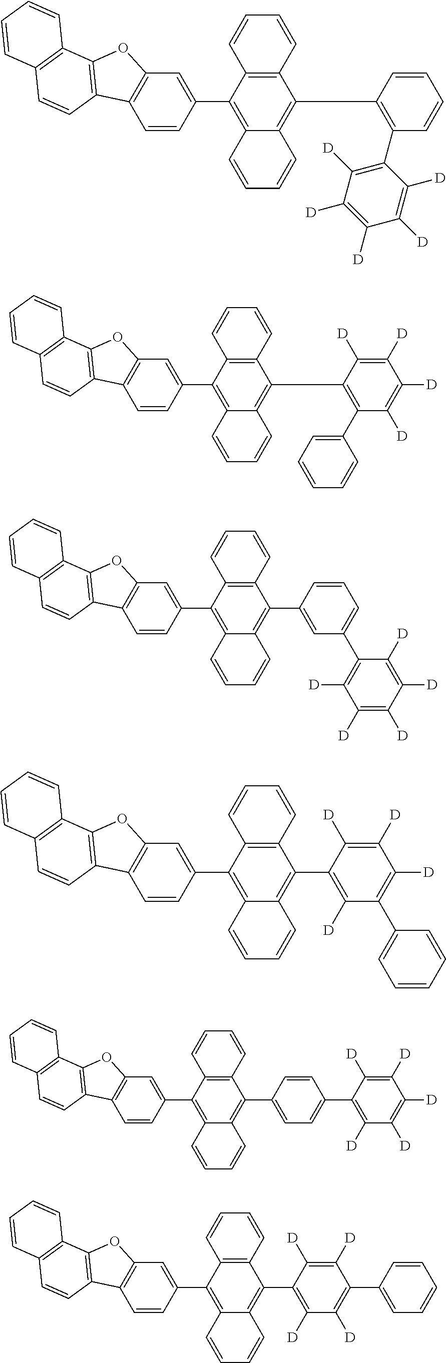

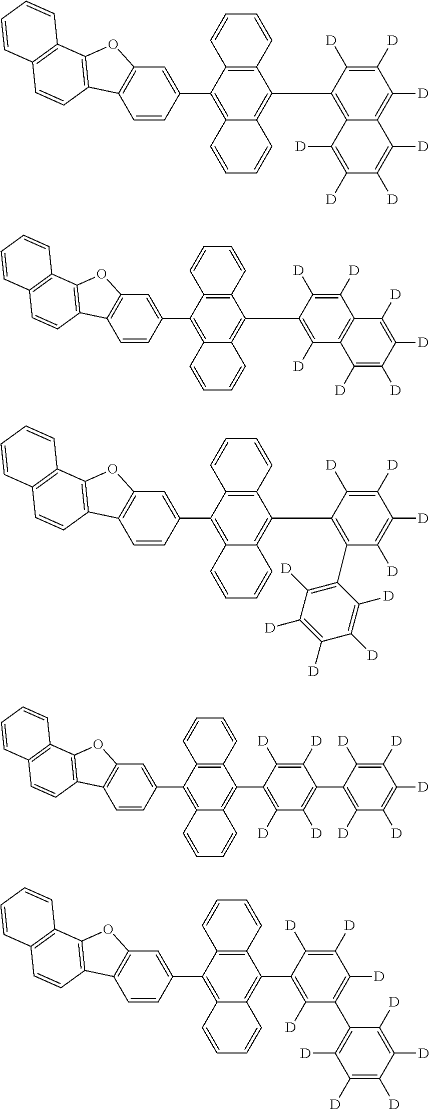

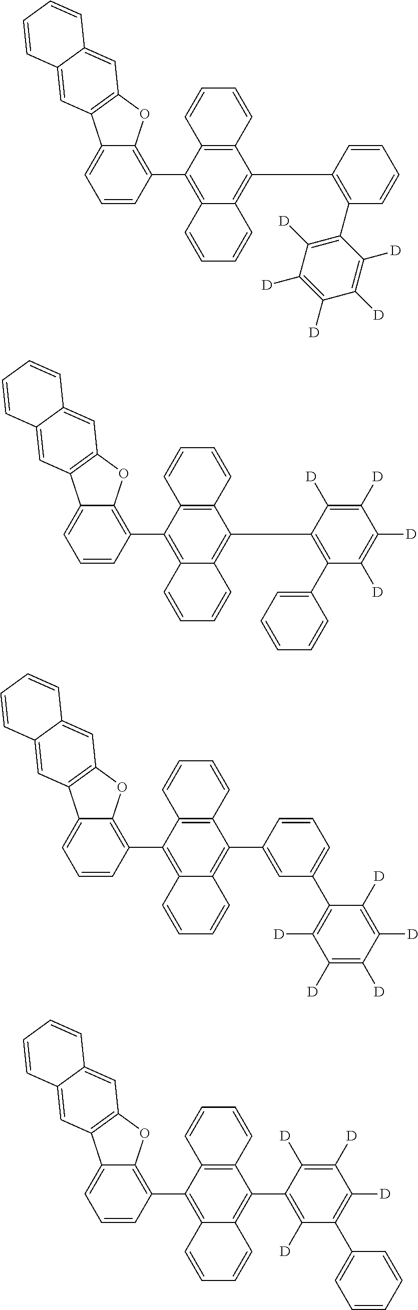 [SMILES: [2H]c1c([2H])c([2H])c(-c2ccccc2-c2c3ccccc3c(-c3cccc4c3oc3cc5ccccc5cc34)c3ccccc23)c([2H])c1[2H] ; [2H]c1c([2H])c([2H])c(-c2c3ccccc3c(-c3cccc4c3oc3cc5ccccc5cc34)c3ccccc23)c(-c2ccccc2)c1[2H] ; [2H]c1c([2H])c([2H])c(-c2cccc(-c3c4ccccc4c(-c4cccc5c4oc4cc6ccccc6cc45)c4ccccc34)c2)c([2H])c1[2H] ; [2H]c1c([2H])c(-c2ccccc2)c([2H])c(-c2c3ccccc3c(-c3cccc4c3oc3cc5ccccc5cc34)c3ccccc23)c1[2H]]

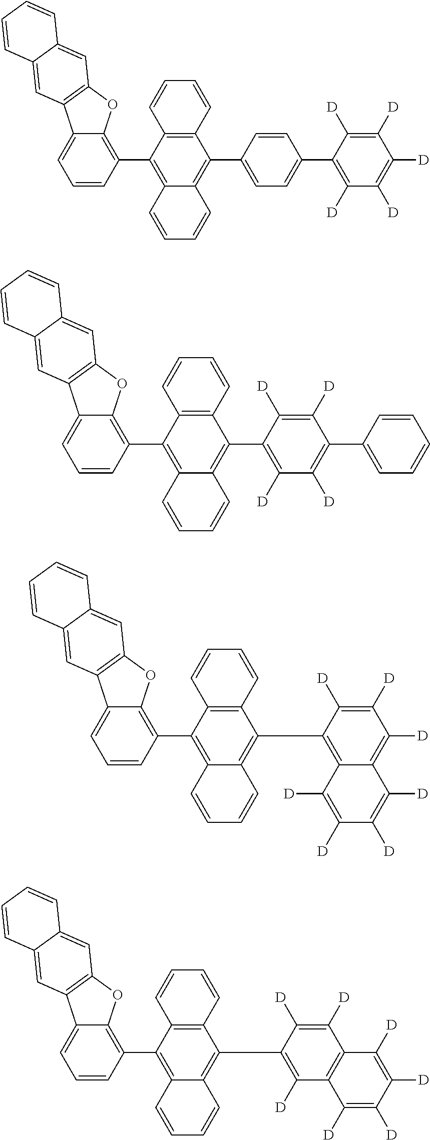 [SMILES: [2H]c1c([2H])c([2H])c(-c2ccc(-c3c4ccccc4c(-c4cccc5c4oc4cc6ccccc6cc45)c4ccccc34)cc2)c([2H])c1[2H] ; [2H]c1c([2H])c(-c2c3ccccc3c(-c3cccc4c3oc3cc5ccccc5cc34)c3ccccc23)c([2H])c([2H])c1-c1ccccc1 ; [2H]c1c([2H])c([2H])c2c(-c3c4ccccc4c(-c4cccc5c4oc4cc6ccccc6cc45)c4ccccc34)c([2H])c([2H])c([2H])c2c1[2H] ; [2H]c1c([2H])c([2H])c2c([2H])c(-c3c4ccccc4c(-c4cccc5c4oc4cc6ccccc6cc45)c4ccccc34)c([2H])c([2H])c2c1[2H]]

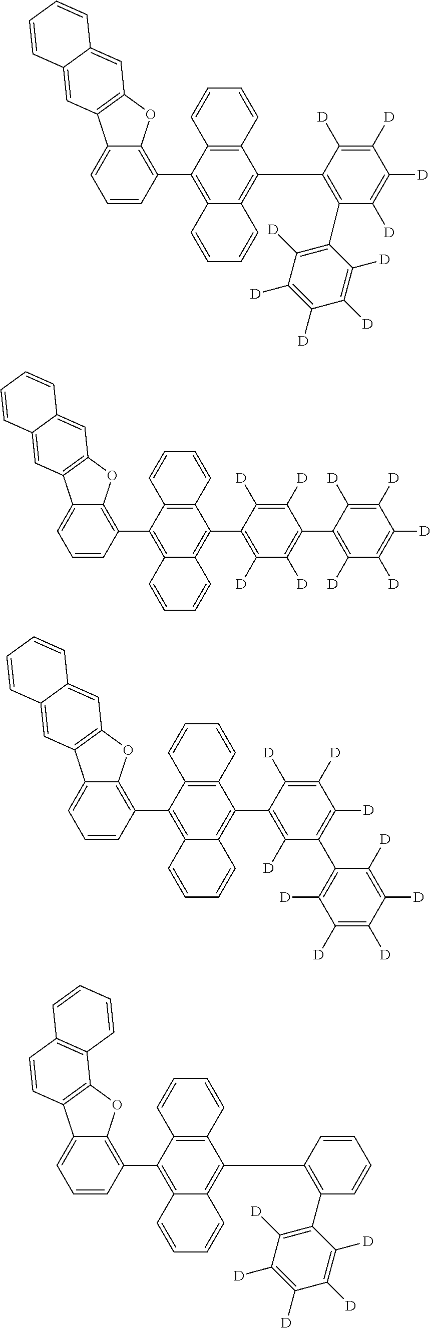 [SMILES: [2H]c1c([2H])c([2H])c(-c2c([2H])c([2H])c([2H])c([2H])c2-c2c3ccccc3c(-c3cccc4c3oc3cc5ccccc5cc34)c3ccccc23)c([2H])c1[2H] ; [2H]c1c([2H])c([2H])c(-c2c([2H])c([2H])c(-c3c4ccccc4c(-c4cccc5c4oc4cc6ccccc6cc45)c4ccccc34)c([2H])c2[2H])c([2H])c1[2H] ; [2H]c1c([2H])c([2H])c(-c2c([2H])c([2H])c([2H])c(-c3c4ccccc4c(-c4cccc5c4oc4cc6ccccc6cc45)c4ccccc34)c2[2H])c([2H])c1[2H] ; [2H]c1c([2H])c([2H])c(-c2ccccc2-c2c3ccccc3c(-c3cccc4c3oc3c5ccccc5ccc43)c3ccccc23)c([2H])c1[2H]]

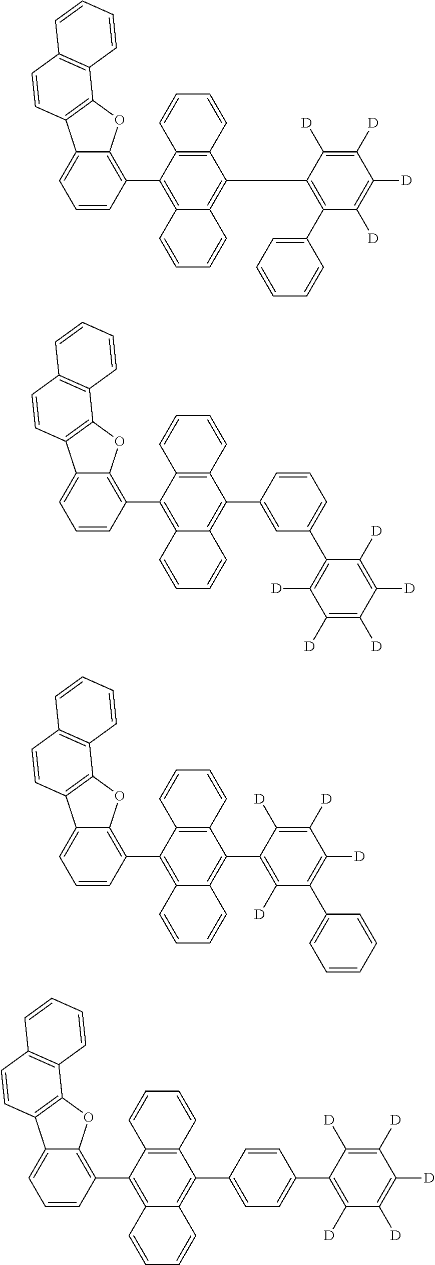 [SMILES: [2H]c1c([2H])c([2H])c(-c2c3ccccc3c(-c3cccc4c3oc3c5ccccc5ccc43)c3ccccc23)c(-c2ccccc2)c1[2H] ; [2H]c1c([2H])c([2H])c(-c2cccc(-c3c4ccccc4c(-c4cccc5c4oc4c6ccccc6ccc54)c4ccccc34)c2)c([2H])c1[2H] ; [2H]c1c([2H])c(-c2ccccc2)c([2H])c(-c2c3ccccc3c(-c3cccc4c3oc3c5ccccc5ccc43)c3ccccc23)c1[2H] ; [2H]c1c([2H])c([2H])c(-c2ccc(-c3c4ccccc4c(-c4cccc5c4oc4c6ccccc6ccc54)c4ccccc34)cc2)c([2H])c1[2H]]

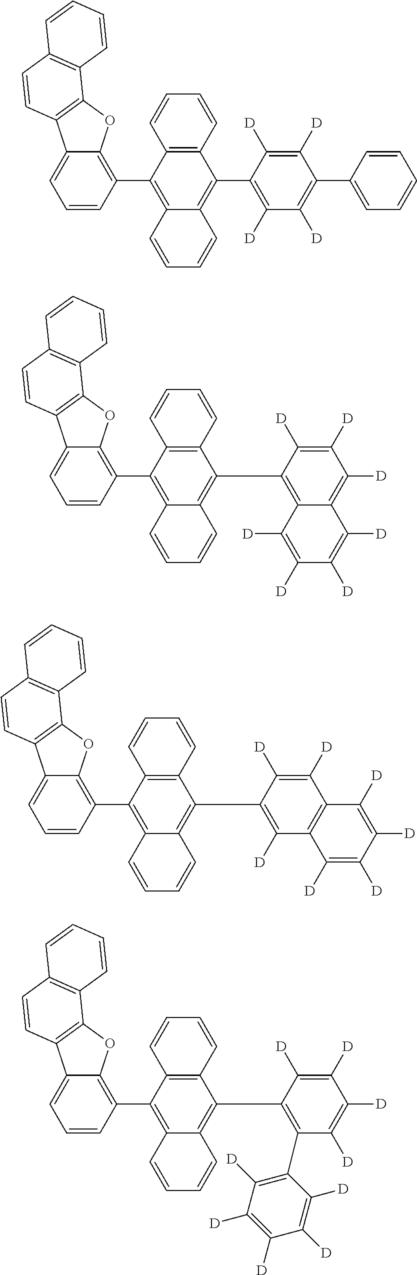 [SMILES: [2H]c1c([2H])c(-c2c3ccccc3c(-c3cccc4c3oc3c5ccccc5ccc43)c3ccccc23)c([2H])c([2H])c1-c1ccccc1 ; [2H]c1c([2H])c([2H])c2c(-c3c4ccccc4c(-c4cccc5c4oc4c6ccccc6ccc54)c4ccccc34)c([2H])c([2H])c([2H])c2c1[2H] ; [2H]c1c([2H])c([2H])c2c([2H])c(-c3c4ccccc4c(-c4cccc5c4oc4c6ccccc6ccc54)c4ccccc34)c([2H])c([2H])c2c1[2H] ; [2H]c1c([2H])c([2H])c(-c2c([2H])c([2H])c([2H])c([2H])c2-c2c3ccccc3c(-c3cccc4c3oc3c5ccccc5ccc43)c3ccccc23)c([2H])c1[2H]]

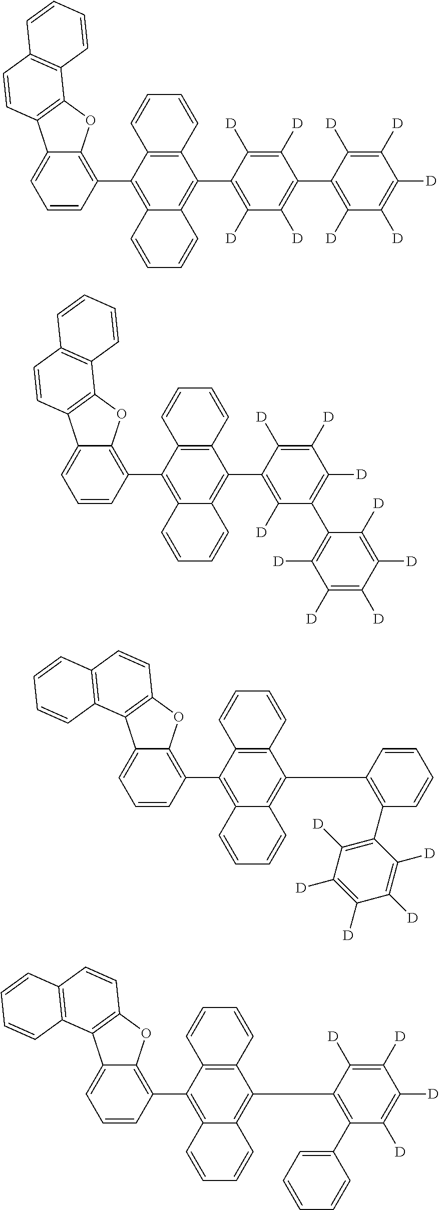 [SMILES: [2H]c1c([2H])c([2H])c(-c2c([2H])c([2H])c(-c3c4ccccc4c(-c4cccc5c4oc4c6ccccc6ccc54)c4ccccc34)c([2H])c2[2H])c([2H])c1[2H] ; [2H]c1c([2H])c([2H])c(-c2c([2H])c([2H])c([2H])c(-c3c4ccccc4c(-c4cccc5c4oc4c6ccccc6ccc54)c4ccccc34)c2[2H])c([2H])c1[2H] ; [2H]c1c([2H])c([2H])c(-c2ccccc2-c2c3ccccc3c(-c3cccc4c3oc3ccc5ccccc5c34)c3ccccc23)c([2H])c1[2H] ; [2H]c1c([2H])c([2H])c(-c2c3ccccc3c(-c3cccc4c3oc3ccc5ccccc5c34)c3ccccc23)c(-c2ccccc2)c1[2H]]

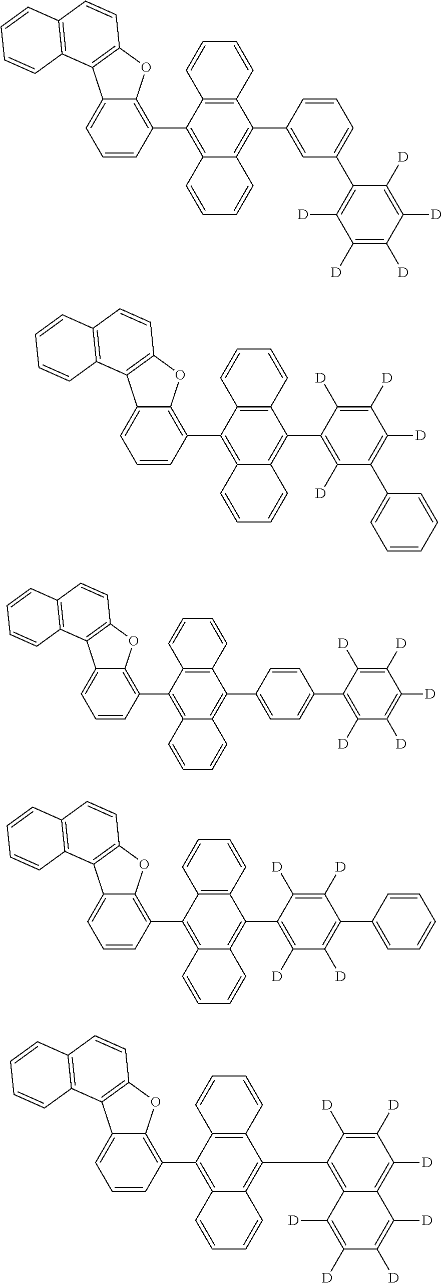 [SMILES: [2H]c1c([2H])c([2H])c(-c2cccc(-c3c4ccccc4c(-c4cccc5c4oc4ccc6ccccc6c45)c4ccccc34)c2)c([2H])c1[2H] ; [2H]c1c([2H])c(-c2ccccc2)c([2H])c(-c2c3ccccc3c(-c3cccc4c3oc3ccc5ccccc5c34)c3ccccc23)c1[2H] ; [2H]c1c([2H])c([2H])c(-c2ccc(-c3c4ccccc4c(-c4cccc5c4oc4ccc6ccccc6c45)c4ccccc34)cc2)c([2H])c1[2H] ; [2H]c1c([2H])c(-c2c3ccccc3c(-c3cccc4c3oc3ccc5ccccc5c34)c3ccccc23)c([2H])c([2H])c1-c1ccccc1 ; [2H]c1c([2H])c([2H])c2c(-c3c4ccccc4c(-c4cccc5c4oc4ccc6ccccc6c45)c4ccccc34)c([2H])c([2H])c([2H])c2c1[2H]]

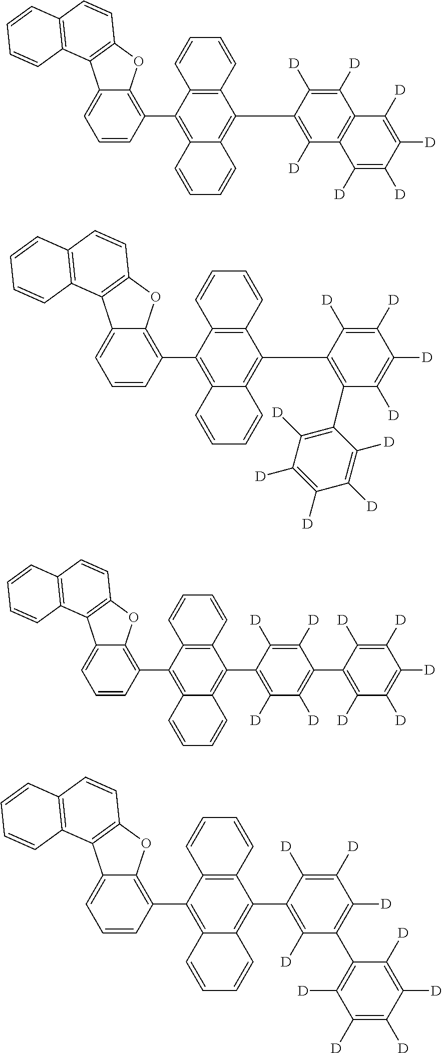 [SMILES: [2H]c1c([2H])c([2H])c2c([2H])c(-c3c4ccccc4c(-c4cccc5c4oc4ccc6ccccc6c45)c4ccccc34)c([2H])c([2H])c2c1[2H] ; [2H]c1c([2H])c([2H])c(-c2c([2H])c([2H])c([2H])c([2H])c2-c2c3ccccc3c(-c3cccc4c3oc3ccc5ccccc5c34)c3ccccc23)c([2H])c1[2H] ; [2H]c1c([2H])c([2H])c(-c2c([2H])c([2H])c(-c3c4ccccc4c(-c4cccc5c4oc4ccc6ccccc6c45)c4ccccc34)c([2H])c2[2H])c([2H])c1[2H] ; [2H]c1c([2H])c([2H])c(-c2c([2H])c([2H])c([2H])c(-c3c4ccccc4c(-c4cccc5c4oc4ccc6ccccc6c45)c4ccccc34)c2[2H])c([2H])c1[2H]]

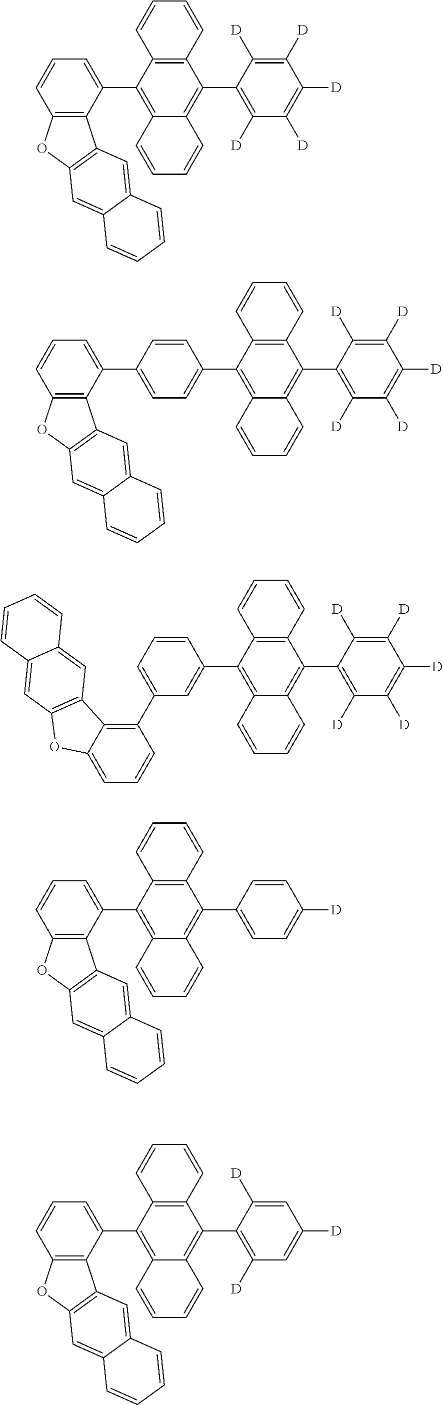

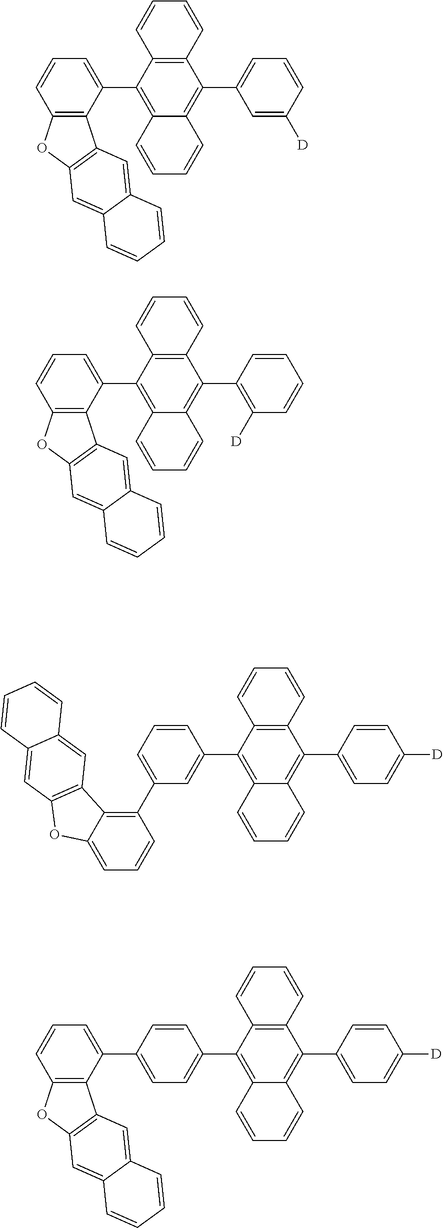

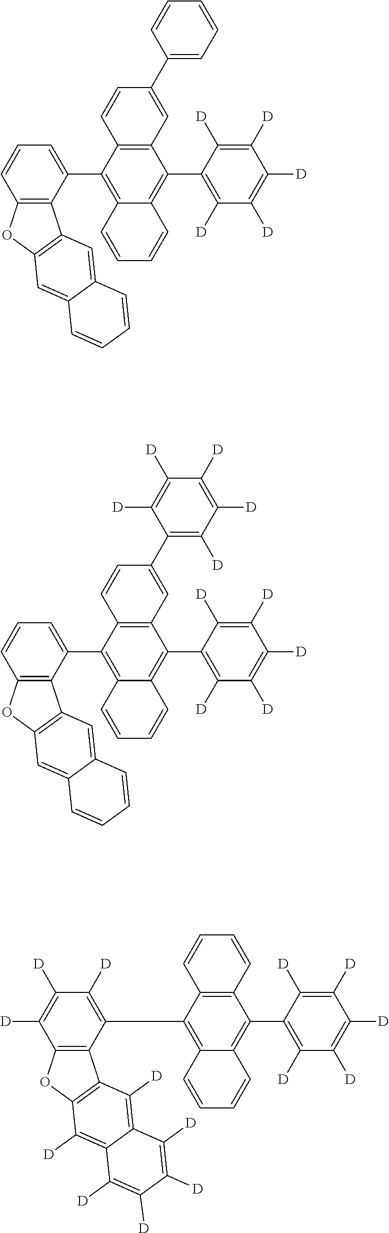 [SMILES: [2H]c1c([2H])c([2H])c(-c2c3ccccc3c(-c3cccc4oc5cc6ccccc6cc5c34)c3ccc(-c4ccccc4)cc23)c([2H])c1[2H] ; [2H]c1c([2H])c([2H])c(-c2ccc3c(-c4cccc5oc6cc7ccccc7cc6c45)c4ccccc4c(-c4c([2H])c([2H])c([2H])c([2H])c4[2H])c3c2)c([2H])c1[2H] ; [2H]c1c([2H])c([2H])c(-c2c3ccccc3c(-c3c([2H])c([2H])c([2H])c4oc5c([2H])c6c([2H])c([2H])c([2H])c([2H])c6c([2H])c5c34)c3ccccc23)c([2H])c1[2H]]

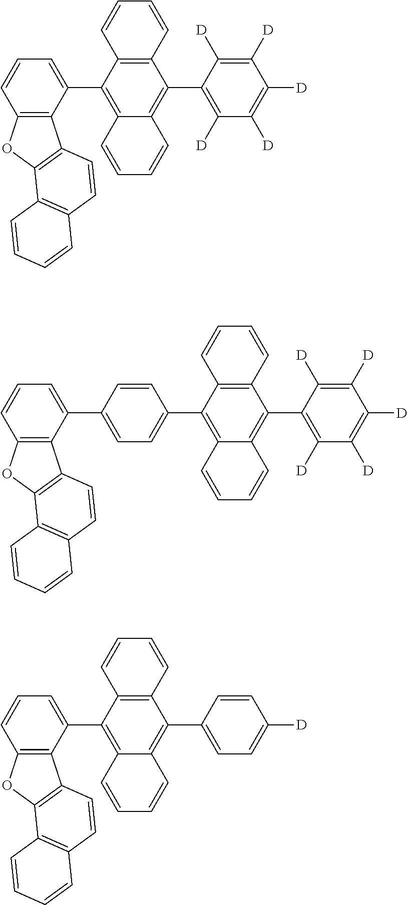 [SMILES: [2H]c1c([2H])c([2H])c(-c2c3ccccc3c(-c3cccc4oc5c6ccccc6ccc5c34)c3ccccc23)c([2H])c1[2H] ; [2H]c1c([2H])c([2H])c(-c2c3ccccc3c(-c3ccc(-c4cccc5oc6c7ccccc7ccc6c45)cc3)c3ccccc23)c([2H])c1[2H] ; [2H]c1ccc(-c2c3ccccc3c(-c3cccc4oc5c6ccccc6ccc5c34)c3ccccc23)cc1]

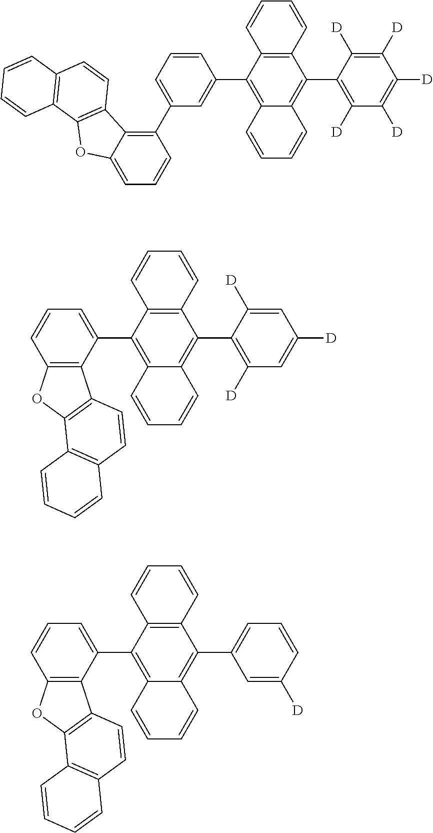 [SMILES: [2H]c1c([2H])c([2H])c(-c2c3ccccc3c(-c3cccc(-c4cccc5oc6c7ccccc7ccc6c45)c3)c3ccccc23)c([2H])c1[2H] ; [2H]c1cc([2H])c(-c2c3ccccc3c(-c3cccc4oc5c6ccccc6ccc5c34)c3ccccc23)c([2H])c1 ; [2H]c1cccc(-c2c3ccccc3c(-c3cccc4oc5c6ccccc6ccc5c34)c3ccccc23)c1]

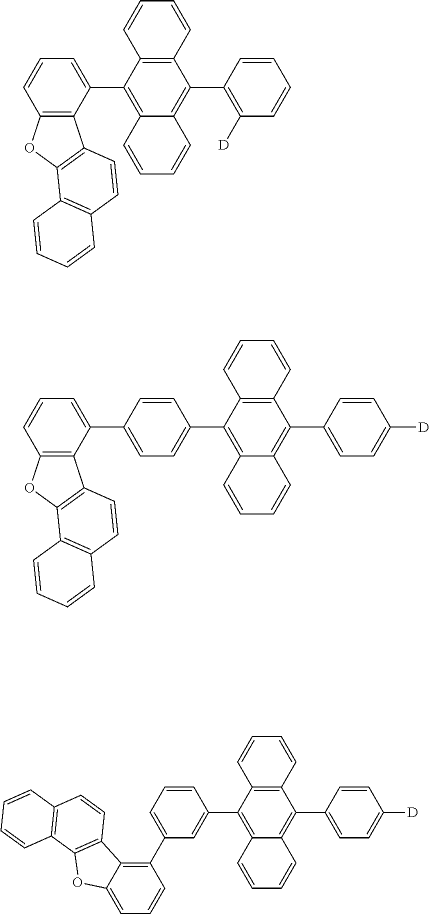 [SMILES: [2H]c1ccccc1-c1c2ccccc2c(-c2cccc3oc4c5ccccc5ccc4c23)c2ccccc12 ; [2H]c1ccc(-c2c3ccccc3c(-c3ccc(-c4cccc5oc6c7ccccc7ccc6c45)cc3)c3ccccc23)cc1 ; [2H]c1ccc(-c2c3ccccc3c(-c3cccc(-c4cccc5oc6c7ccccc7ccc6c45)c3)c3ccccc23)cc1]

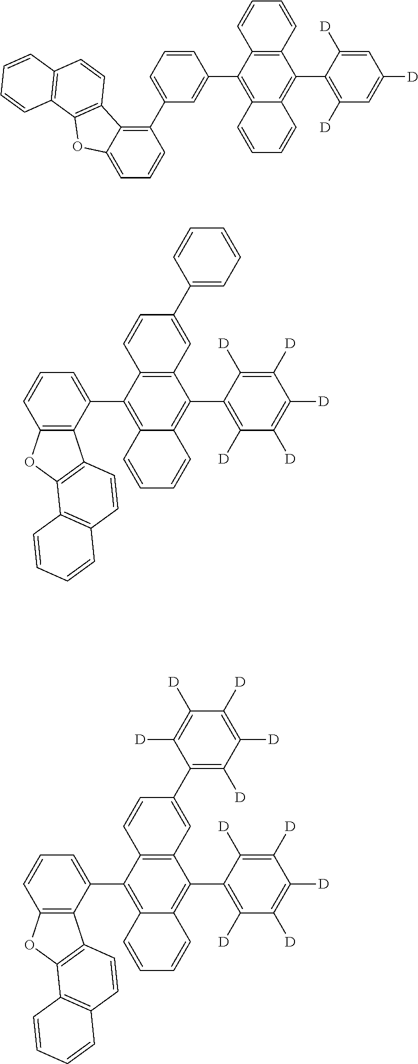 [SMILES: [2H]c1cc([2H])c(-c2c3ccccc3c(-c3cccc(-c4cccc5oc6c7ccccc7ccc6c45)c3)c3ccccc23)c([2H])c1 ; [2H]c1c([2H])c([2H])c(-c2c3ccccc3c(-c3cccc4oc5c6ccccc6ccc5c34)c3ccc(-c4ccccc4)cc23)c([2H])c1[2H] ; [2H]c1c([2H])c([2H])c(-c2ccc3c(-c4cccc5oc6c7ccccc7ccc6c45)c4ccccc4c(-c4c([2H])c([2H])c([2H])c([2H])c4[2H])c3c2)c([2H])c1[2H]]

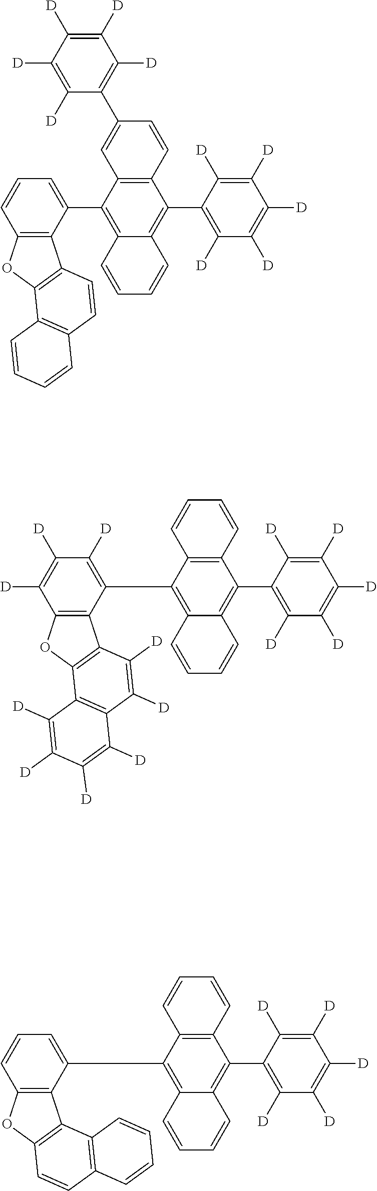 [SMILES: [2H]c1c([2H])c([2H])c(-c2ccc3c(-c4c([2H])c([2H])c([2H])c([2H])c4[2H])c4ccccc4c(-c4cccc5oc6c7ccccc7ccc6c45)c3c2)c([2H])c1[2H] ; [2H]c1c([2H])c([2H])c(-c2c3ccccc3c(-c3c([2H])c([2H])c([2H])c4oc5c6c([2H])c([2H])c([2H])c([2H])c6c([2H])c([2H])c5c34)c3ccccc23)c([2H])c1[2H] ; [2H]c1c([2H])c([2H])c(-c2c3ccccc3c(-c3cccc4oc5ccc6ccccc6c5c34)c3ccccc23)c([2H])c1[2H]]

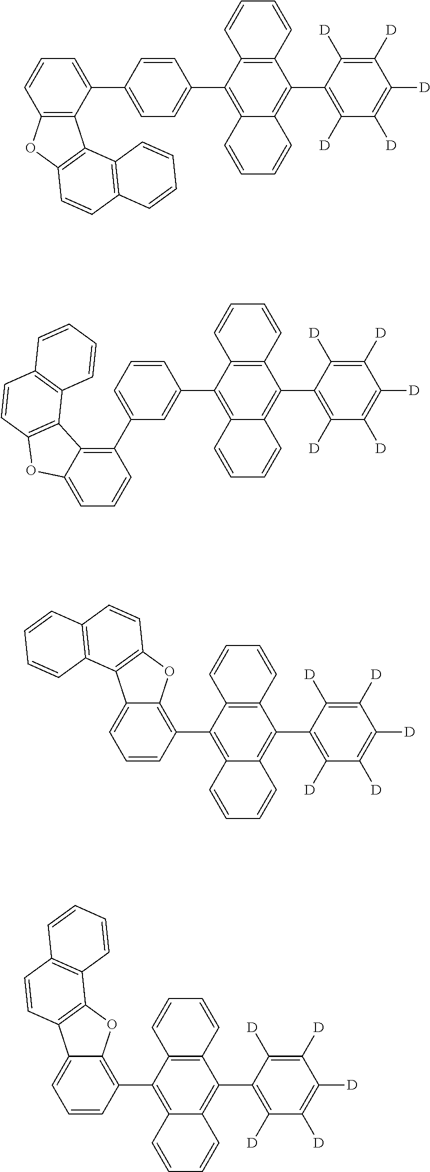 [SMILES: [2H]c1c([2H])c([2H])c(-c2c3ccccc3c(-c3ccc(-c4cccc5oc6ccc7ccccc7c6c45)cc3)c3ccccc23)c([2H])c1[2H] ; [2H]c1c([2H])c([2H])c(-c2c3ccccc3c(-c3cccc(-c4cccc5oc6ccc7ccccc7c6c45)c3)c3ccccc23)c([2H])c1[2H] ; [2H]c1c([2H])c([2H])c(-c2c3ccccc3c(-c3cccc4c3oc3ccc5ccccc5c34)c3ccccc23)c([2H])c1[2H] ; [2H]c1c([2H])c([2H])c(-c2c3ccccc3c(-c3cccc4c3oc3c5ccccc5ccc43)c3ccccc23)c([2H])c1[2H]]

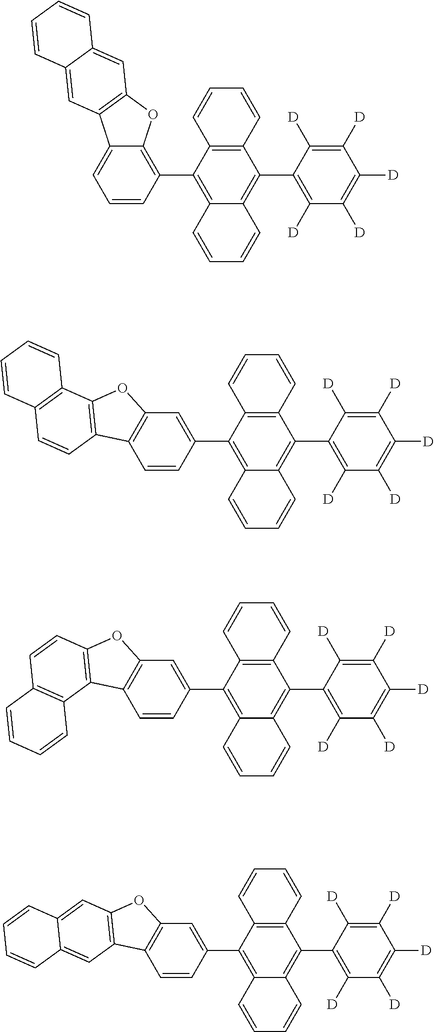 [SMILES: [2H]c1c([2H])c([2H])c(-c2c3ccccc3c(-c3cccc4c3oc3cc5ccccc5cc34)c3ccccc23)c([2H])c1[2H] ; [2H]c1c([2H])c([2H])c(-c2c3ccccc3c(-c3ccc4c(c3)oc3c5ccccc5ccc43)c3ccccc23)c([2H])c1[2H] ; [2H]c1c([2H])c([2H])c(-c2c3ccccc3c(-c3ccc4c(c3)oc3ccc5ccccc5c34)c3ccccc23)c([2H])c1[2H] ; [2H]c1c([2H])c([2H])c(-c2c3ccccc3c(-c3ccc4c(c3)oc3cc5ccccc5cc34)c3ccccc23)c([2H])c1[2H]]

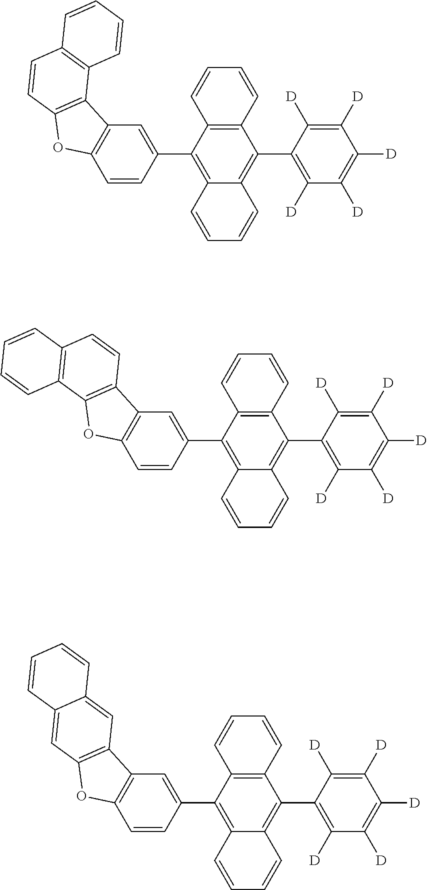 [SMILES: [2H]c1c([2H])c([2H])c(-c2c3ccccc3c(-c3ccc4oc5ccc6ccccc6c5c4c3)c3ccccc23)c([2H])c1[2H] ; [2H]c1c([2H])c([2H])c(-c2c3ccccc3c(-c3ccc4oc5c6ccccc6ccc5c4c3)c3ccccc23)c([2H])c1[2H] ; [2H]c1c([2H])c([2H])c(-c2c3ccccc3c(-c3ccc4oc5cc6ccccc6cc5c4c3)c3ccccc23)c([2H])c1[2H]]

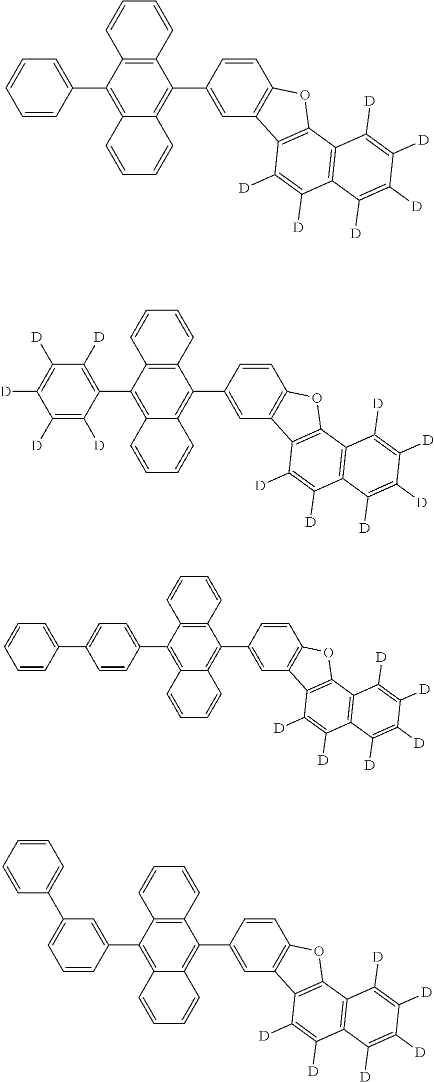 [SMILES: [2H]c1c([2H])c([2H])c2c(c1[2H])c([2H])c([2H])c1c3cc(-c4c5ccccc5c(-c5ccccc5)c5ccccc45)ccc3oc21 ; [2H]c1c([2H])c([2H])c(-c2c3ccccc3c(-c3ccc4oc5c6c([2H])c([2H])c([2H])c([2H])c6c([2H])c([2H])c5c4c3)c3ccccc23)c([2H])c1[2H] ; [2H]c1c([2H])c([2H])c2c(c1[2H])c([2H])c([2H])c1c3cc(-c4c5ccccc5c(-c5ccc(-c6ccccc6)cc5)c5ccccc45)ccc3oc21 ; [2H]c1c([2H])c([2H])c2c(c1[2H])c([2H])c([2H])c1c3cc(-c4c5ccccc5c(-c5cccc(-c6ccccc6)c5)c5ccccc45)ccc3oc21]

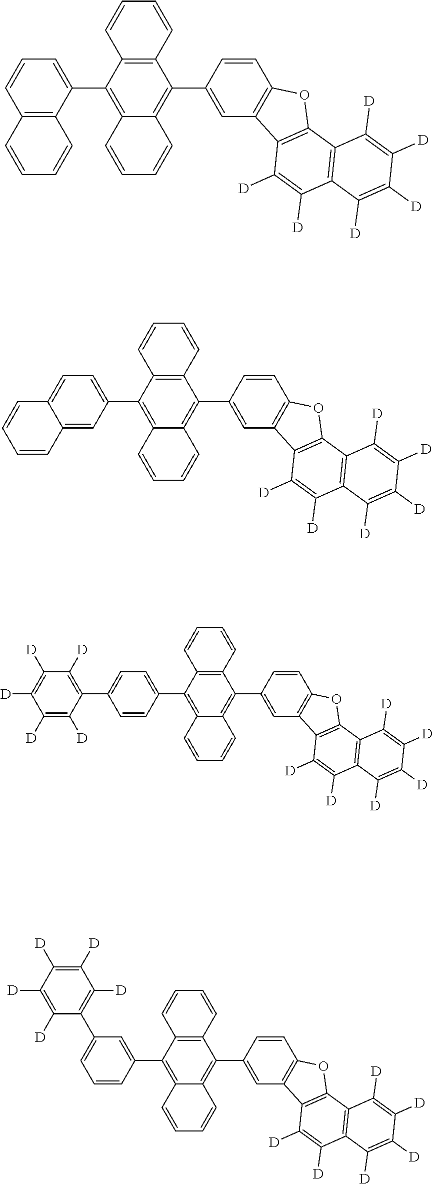 [SMILES: [2H]c1c([2H])c([2H])c2c(c1[2H])c([2H])c([2H])c1c3cc(-c4c5ccccc5c(-c5cccc6ccccc56)c5ccccc45)ccc3oc21 ; [2H]c1c([2H])c([2H])c2c(c1[2H])c([2H])c([2H])c1c3cc(-c4c5ccccc5c(-c5ccc6ccccc6c5)c5ccccc45)ccc3oc21 ; [2H]c1c([2H])c([2H])c(-c2ccc(-c3c4ccccc4c(-c4ccc5oc6c7c([2H])c([2H])c([2H])c([2H])c7c([2H])c([2H])c6c5c4)c4ccccc34)cc2)c([2H])c1[2H] ; [2H]c1c([2H])c([2H])c(-c2cccc(-c3c4ccccc4c(-c4ccc5oc6c7c([2H])c([2H])c([2H])c([2H])c7c([2H])c([2H])c6c5c4)c4ccccc34)c2)c([2H])c1[2H]]

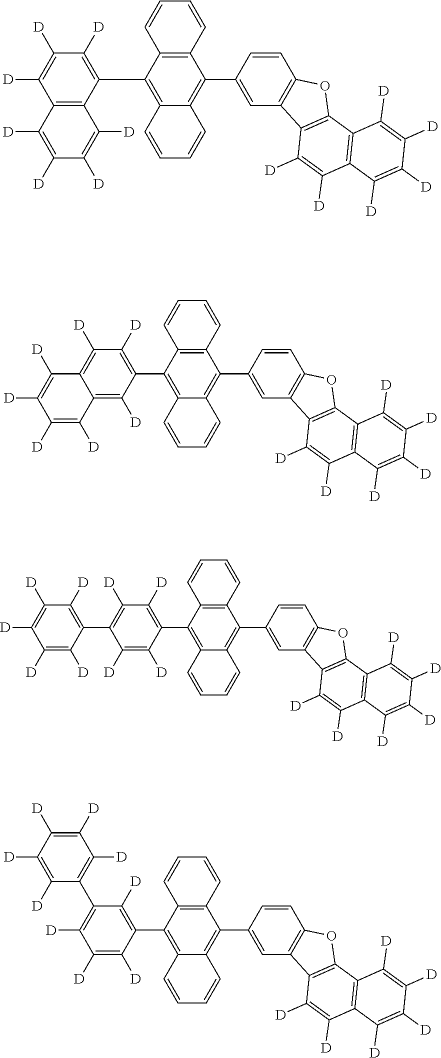

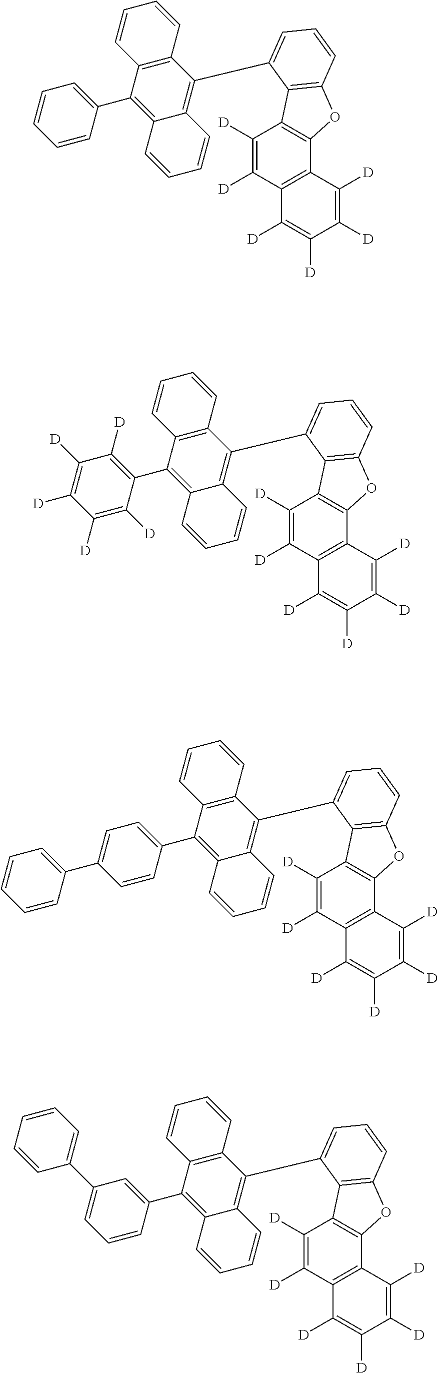 [SMILES: [2H]c1c([2H])c([2H])c2c(c1[2H])c([2H])c([2H])c1c2oc2cccc(-c3c4ccccc4c(-c4ccccc4)c4ccccc34)c21 ; [2H]c1c([2H])c([2H])c(-c2c3ccccc3c(-c3cccc4oc5c6c([2H])c([2H])c([2H])c([2H])c6c([2H])c([2H])c5c34)c3ccccc23)c([2H])c1[2H] ; [2H]c1c([2H])c([2H])c2c(c1[2H])c([2H])c([2H])c1c2oc2cccc(-c3c4ccccc4c(-c4ccc(-c5ccccc5)cc4)c4ccccc34)c21 ; [2H]c1c([2H])c([2H])c2c(c1[2H])c([2H])c([2H])c1c2oc2cccc(-c3c4ccccc4c(-c4cccc(-c5ccccc5)c4)c4ccccc34)c21]

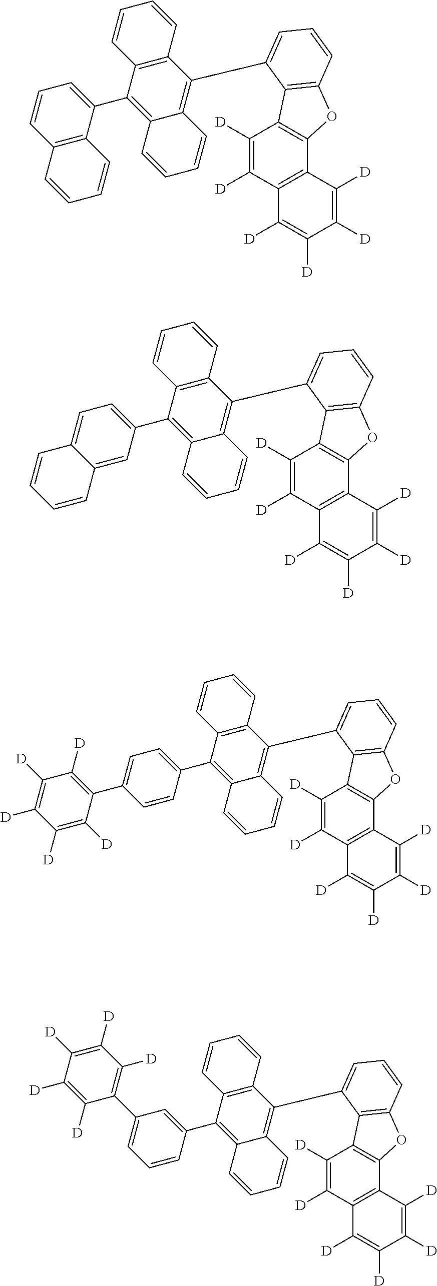 [SMILES: [2H]c1c([2H])c([2H])c2c(c1[2H])c([2H])c([2H])c1c2oc2cccc(-c3c4ccccc4c(-c4cccc5ccccc45)c4ccccc34)c21 ; [2H]c1c([2H])c([2H])c2c(c1[2H])c([2H])c([2H])c1c2oc2cccc(-c3c4ccccc4c(-c4ccc5ccccc5c4)c4ccccc34)c21 ; [2H]c1c([2H])c([2H])c(-c2ccc(-c3c4ccccc4c(-c4cccc5oc6c7c([2H])c([2H])c([2H])c([2H])c7c([2H])c([2H])c6c45)c4ccccc34)cc2)c([2H])c1[2H] ; [2H]c1c([2H])c([2H])c(-c2cccc(-c3c4ccccc4c(-c4cccc5oc6c7c([2H])c([2H])c([2H])c([2H])c7c([2H])c([2H])c6c45)c4ccccc34)c2)c([2H])c1[2H]]

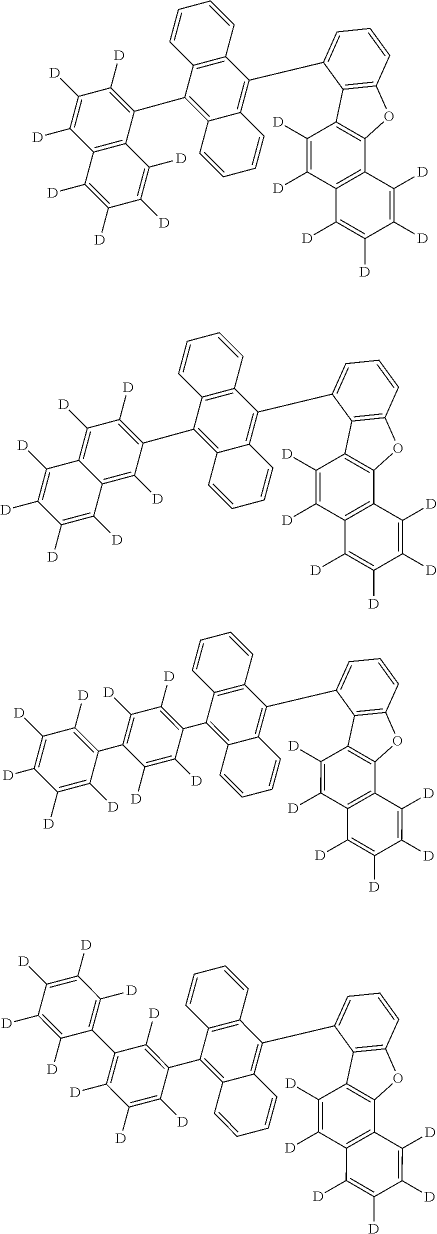

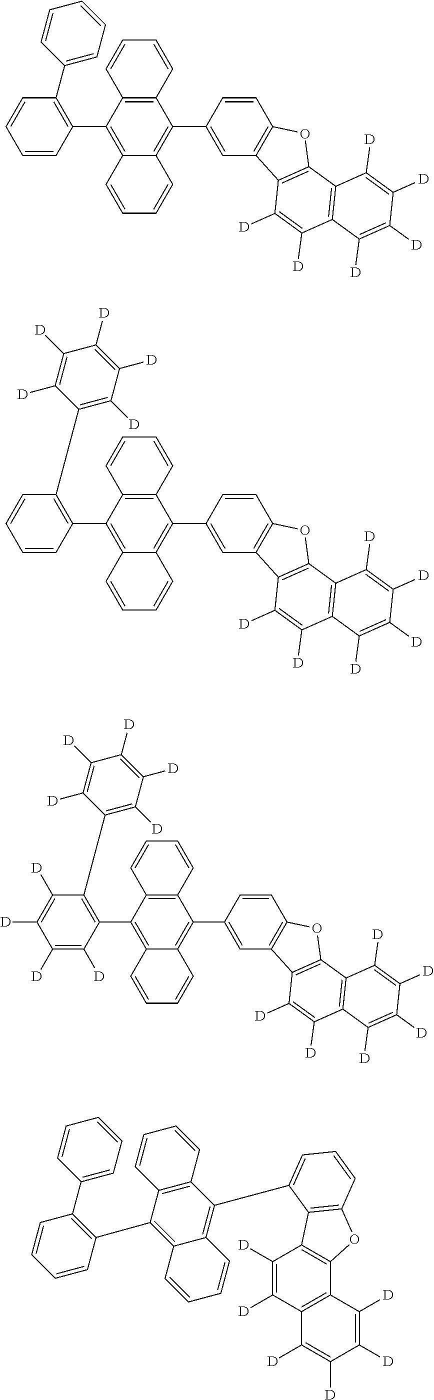 [SMILES: [2H]c1c([2H])c([2H])c2c(c1[2H])c([2H])c([2H])c1c3cc(-c4c5ccccc5c(-c5ccccc5-c5ccccc5)c5ccccc45)ccc3oc21 ; [2H]c1c([2H])c([2H])c(-c2ccccc2-c2c3ccccc3c(-c3ccc4oc5c6c([2H])c([2H])c([2H])c([2H])c6c([2H])c([2H])c5c4c3)c3ccccc23)c([2H])c1[2H] ; [2H]c1c([2H])c([2H])c(-c2c([2H])c([2H])c([2H])c([2H])c2-c2c3ccccc3c(-c3ccc4oc5c6c([2H])c([2H])c([2H])c([2H])c6c([2H])c([2H])c5c4c3)c3ccccc23)c([2H])c1[2H] ; [2H]c1c([2H])c([2H])c2c(c1[2H])c([2H])c([2H])c1c2oc2cccc(-c3c4ccccc4c(-c4ccccc4-c4ccccc4)c4ccccc34)c21]

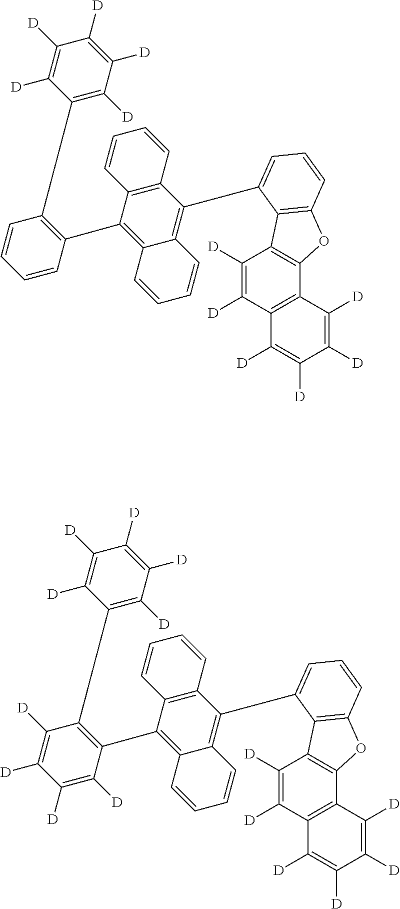

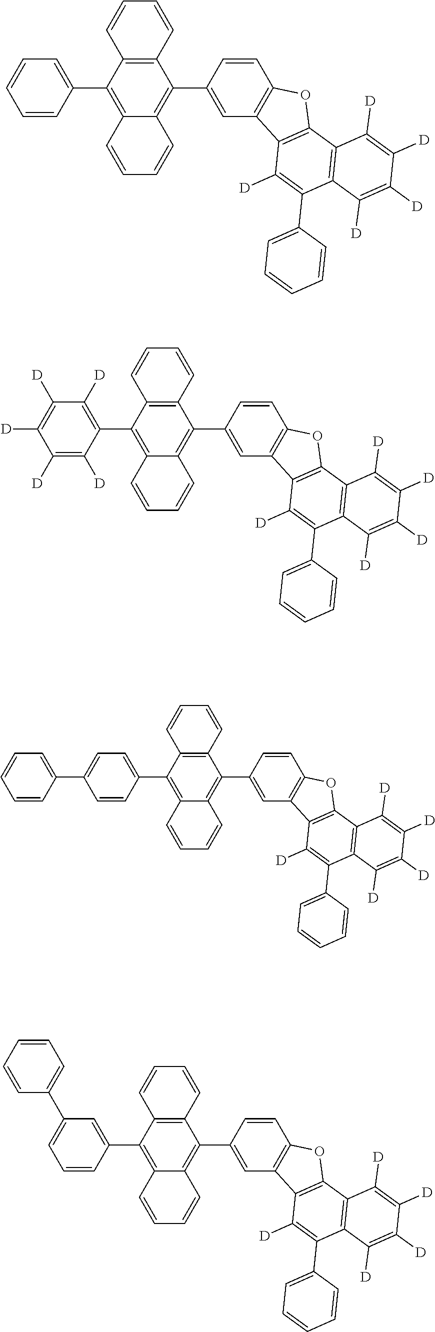 [SMILES: [2H]c1c([2H])c([2H])c2c(c1[2H])c(-c1ccccc1)c([2H])c1c3cc(-c4c5ccccc5c(-c5ccccc5)c5ccccc45)ccc3oc21 ; [2H]c1c([2H])c([2H])c(-c2c3ccccc3c(-c3ccc4oc5c6c([2H])c([2H])c([2H])c([2H])c6c(-c6ccccc6)c([2H])c5c4c3)c3ccccc23)c([2H])c1[2H] ; [2H]c1c([2H])c([2H])c2c(c1[2H])c(-c1ccccc1)c([2H])c1c3cc(-c4c5ccccc5c(-c5ccc(-c6ccccc6)cc5)c5ccccc45)ccc3oc21 ; [2H]c1c([2H])c([2H])c2c(c1[2H])c(-c1ccccc1)c([2H])c1c3cc(-c4c5ccccc5c(-c5cccc(-c6ccccc6)c5)c5ccccc45)ccc3oc21]

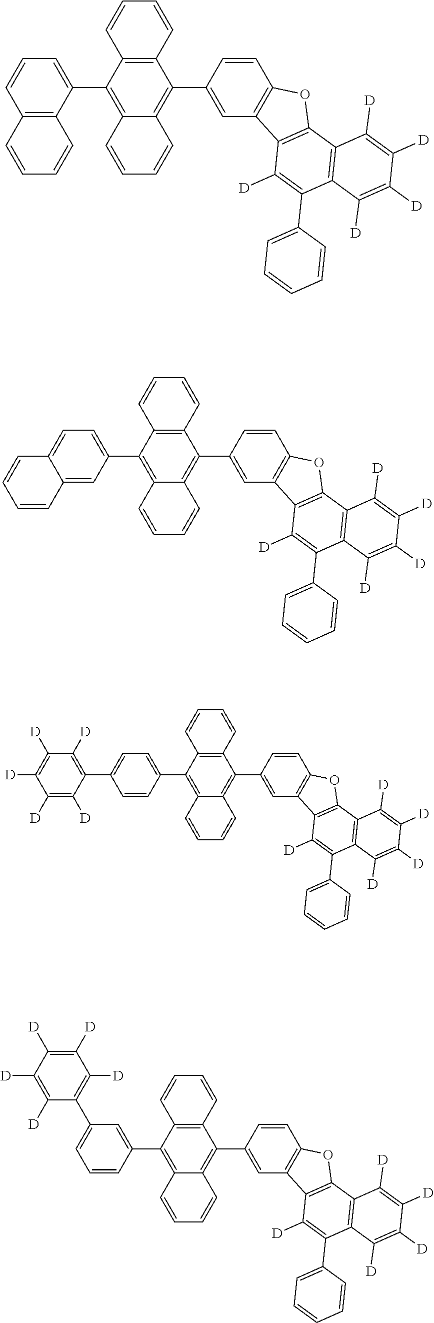 [SMILES: [2H]c1c([2H])c([2H])c2c(c1[2H])c(-c1ccccc1)c([2H])c1c3cc(-c4c5ccccc5c(-c5cccc6ccccc56)c5ccccc45)ccc3oc21 ; [2H]c1c([2H])c([2H])c2c(c1[2H])c(-c1ccccc1)c([2H])c1c3cc(-c4c5ccccc5c(-c5ccc6ccccc6c5)c5ccccc45)ccc3oc21 ; [2H]c1c([2H])c([2H])c(-c2ccc(-c3c4ccccc4c(-c4ccc5oc6c7c([2H])c([2H])c([2H])c([2H])c7c(-c7ccccc7)c([2H])c6c5c4)c4ccccc34)cc2)c([2H])c1[2H] ; [2H]c1c([2H])c([2H])c(-c2cccc(-c3c4ccccc4c(-c4ccc5oc6c7c([2H])c([2H])c([2H])c([2H])c7c(-c7ccccc7)c([2H])c6c5c4)c4ccccc34)c2)c([2H])c1[2H]]

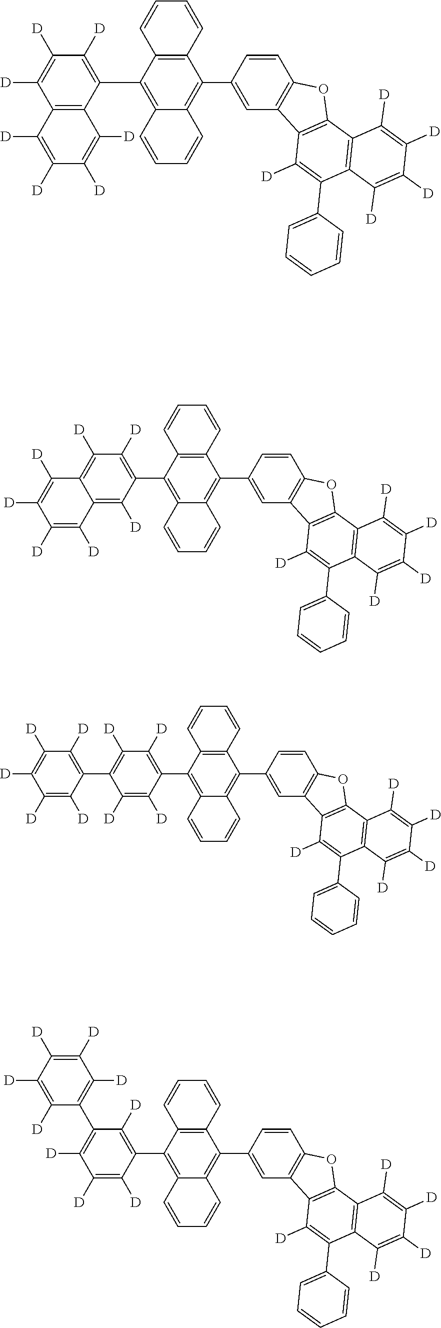

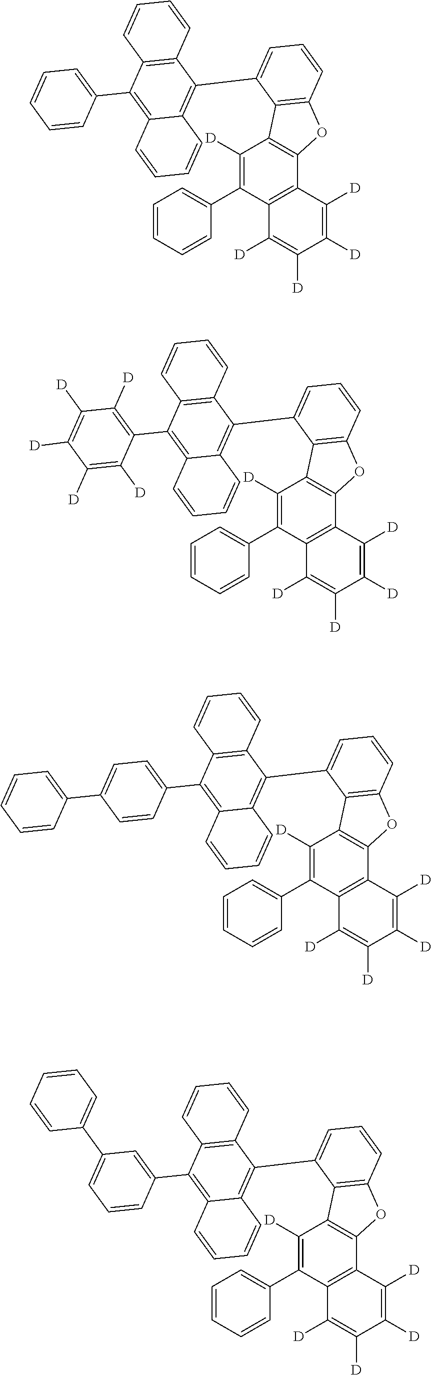 [SMILES: [2H]c1c([2H])c([2H])c2c(c1[2H])c(-c1ccccc1)c([2H])c1c2oc2cccc(-c3c4ccccc4c(-c4ccccc4)c4ccccc34)c21 ; [2H]c1c([2H])c([2H])c(-c2c3ccccc3c(-c3cccc4oc5c6c([2H])c([2H])c([2H])c([2H])c6c(-c6ccccc6)c([2H])c5c34)c3ccccc23)c([2H])c1[2H] ; [2H]c1c([2H])c([2H])c2c(c1[2H])c(-c1ccccc1)c([2H])c1c2oc2cccc(-c3c4ccccc4c(-c4ccc(-c5ccccc5)cc4)c4ccccc34)c21 ; [2H]c1c([2H])c([2H])c2c(c1[2H])c(-c1ccccc1)c([2H])c1c2oc2cccc(-c3c4ccccc4c(-c4cccc(-c5ccccc5)c4)c4ccccc34)c21]

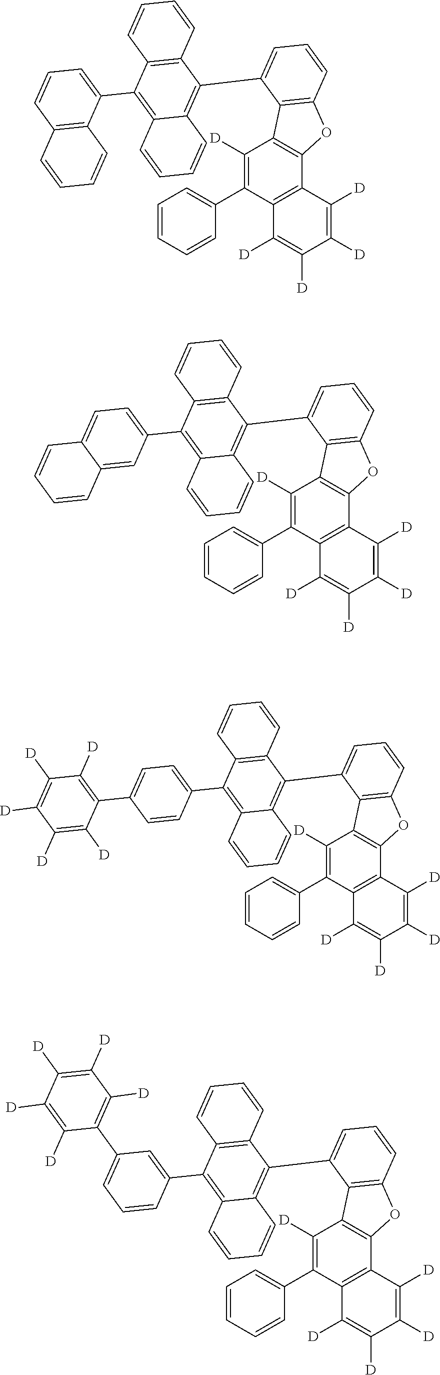 [SMILES: [2H]c1c([2H])c([2H])c2c(c1[2H])c(-c1ccccc1)c([2H])c1c2oc2cccc(-c3c4ccccc4c(-c4cccc5ccccc45)c4ccccc34)c21 ; [2H]c1c([2H])c([2H])c2c(c1[2H])c(-c1ccccc1)c([2H])c1c2oc2cccc(-c3c4ccccc4c(-c4ccc5ccccc5c4)c4ccccc34)c21 ; [2H]c1c([2H])c([2H])c(-c2ccc(-c3c4ccccc4c(-c4cccc5oc6c7c([2H])c([2H])c([2H])c([2H])c7c(-c7ccccc7)c([2H])c6c45)c4ccccc34)cc2)c([2H])c1[2H] ; [2H]c1c([2H])c([2H])c(-c2cccc(-c3c4ccccc4c(-c4cccc5oc6c7c([2H])c([2H])c([2H])c([2H])c7c(-c7ccccc7)c([2H])c6c45)c4ccccc34)c2)c([2H])c1[2H]]

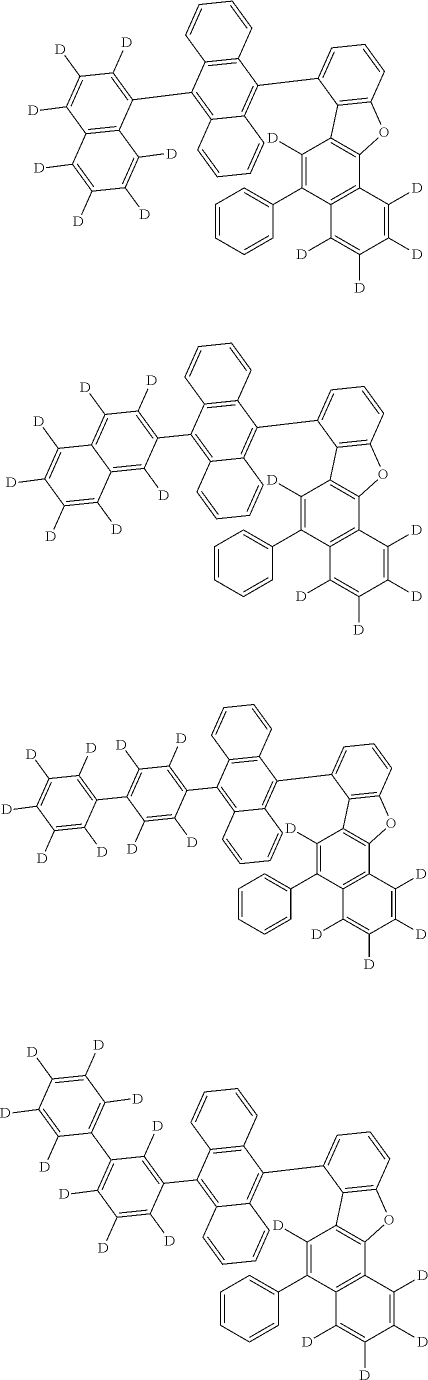

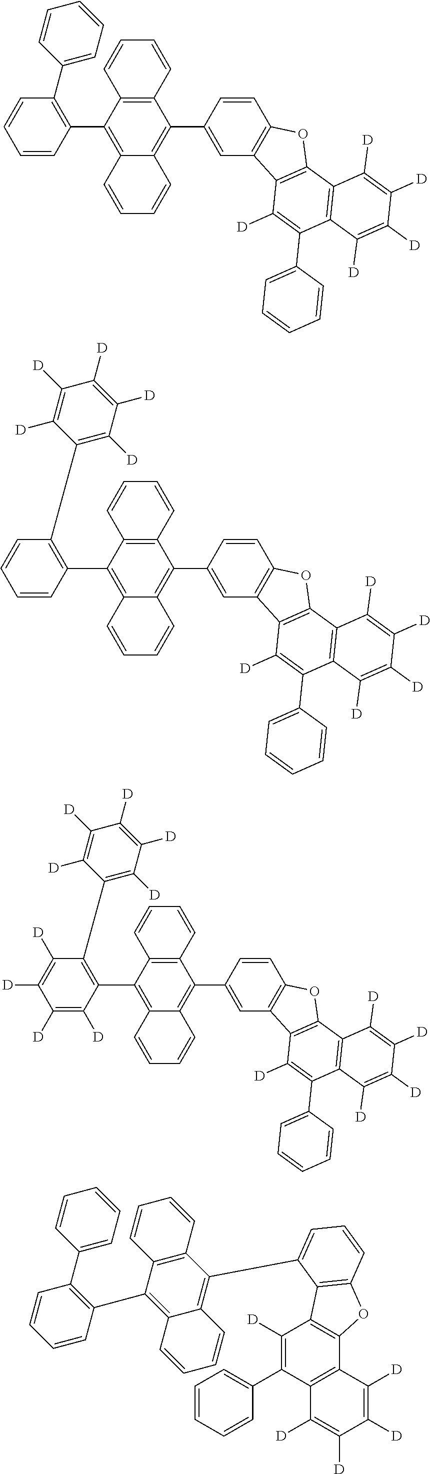

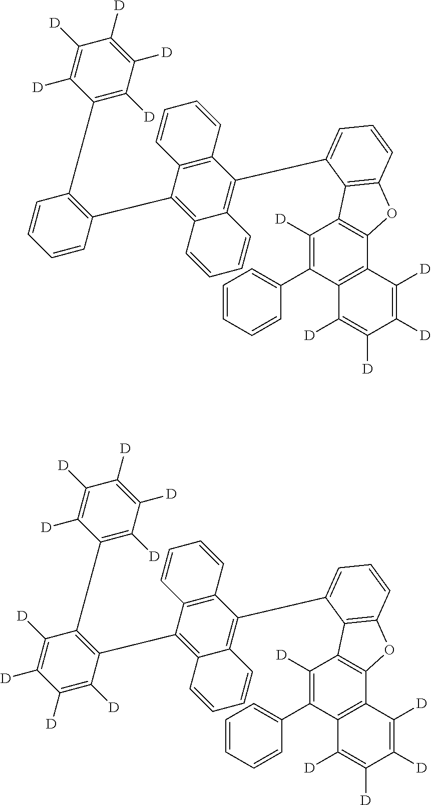 [SMILES: [2H]c1c([2H])c([2H])c(-c2ccccc2-c2c3ccccc3c(-c3cccc4oc5c6c([2H])c([2H])c([2H])c([2H])c6c(-c6ccccc6)c([2H])c5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5c6c([2H])c([2H])c([2H])c([2H])c6c(-c6ccccc6)c([2H])c5c34)c3ccccc23)c([2H])c1[2H]]

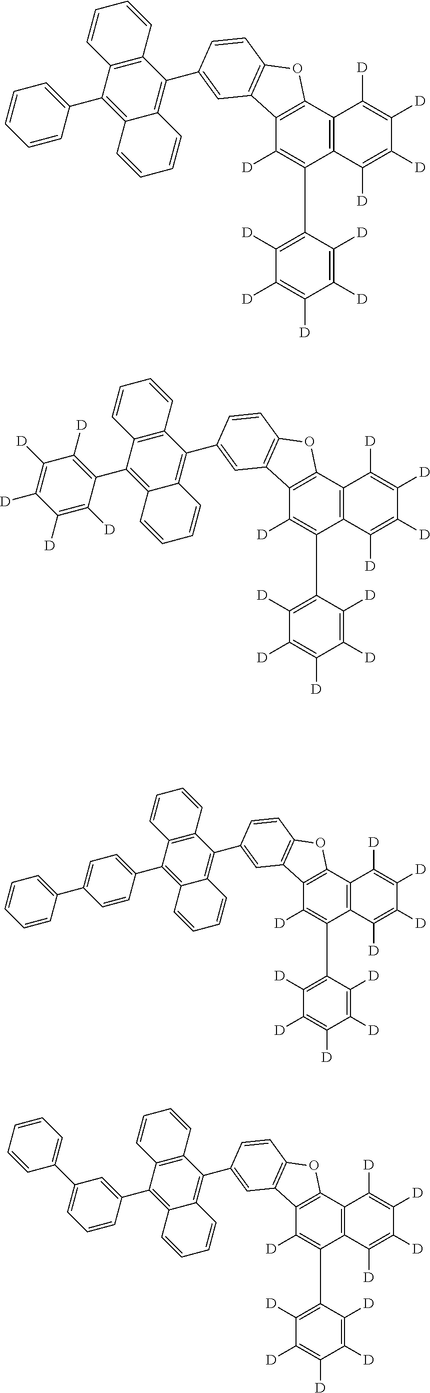

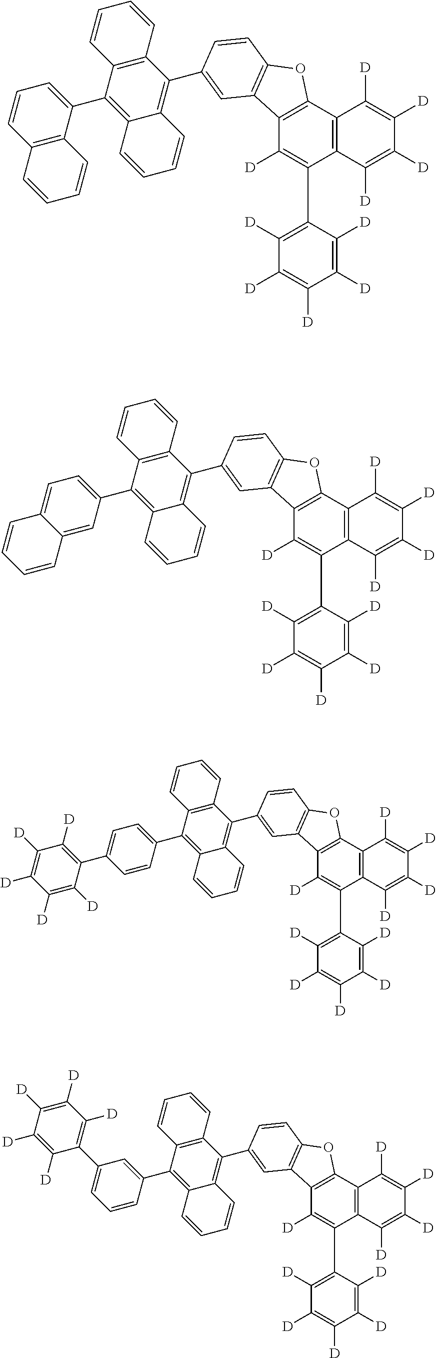

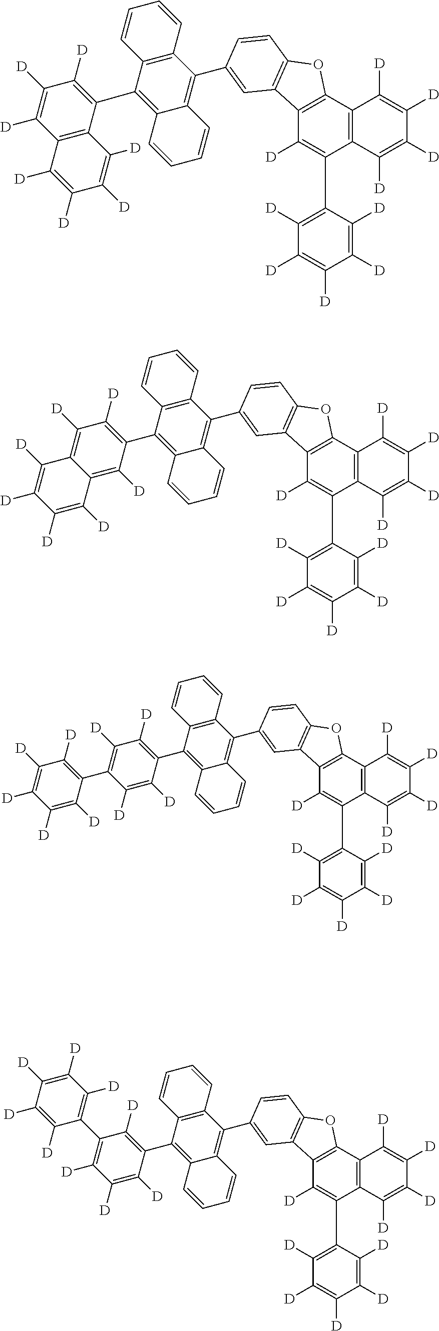

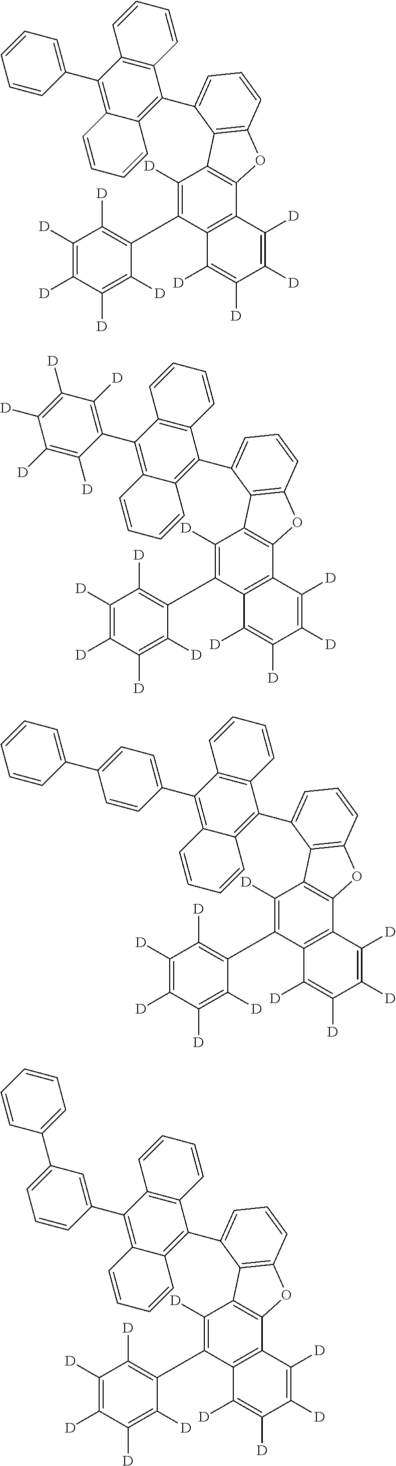

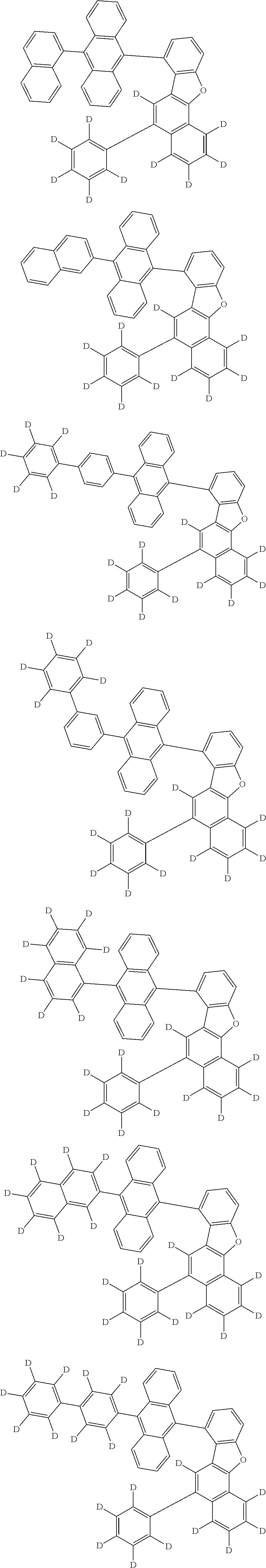

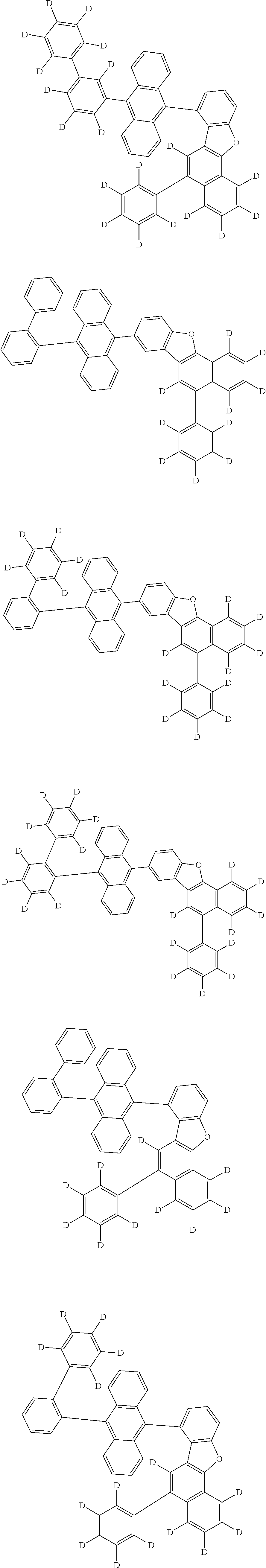

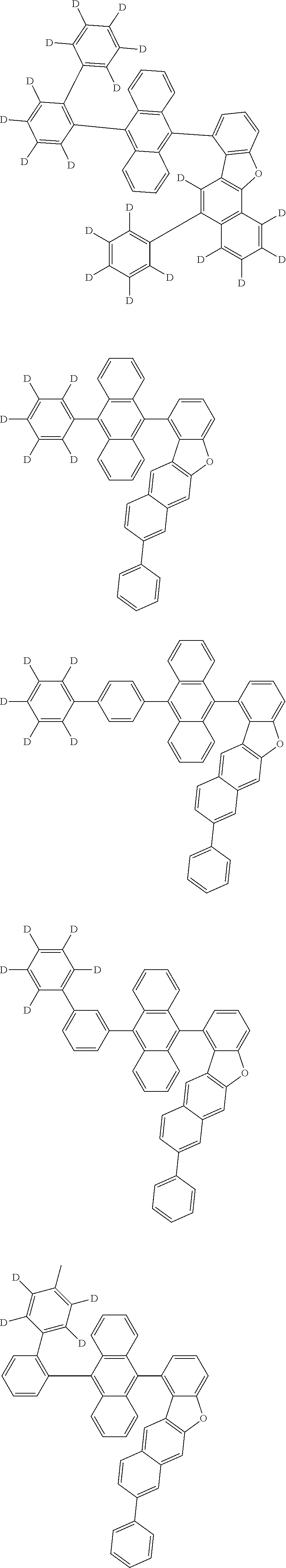

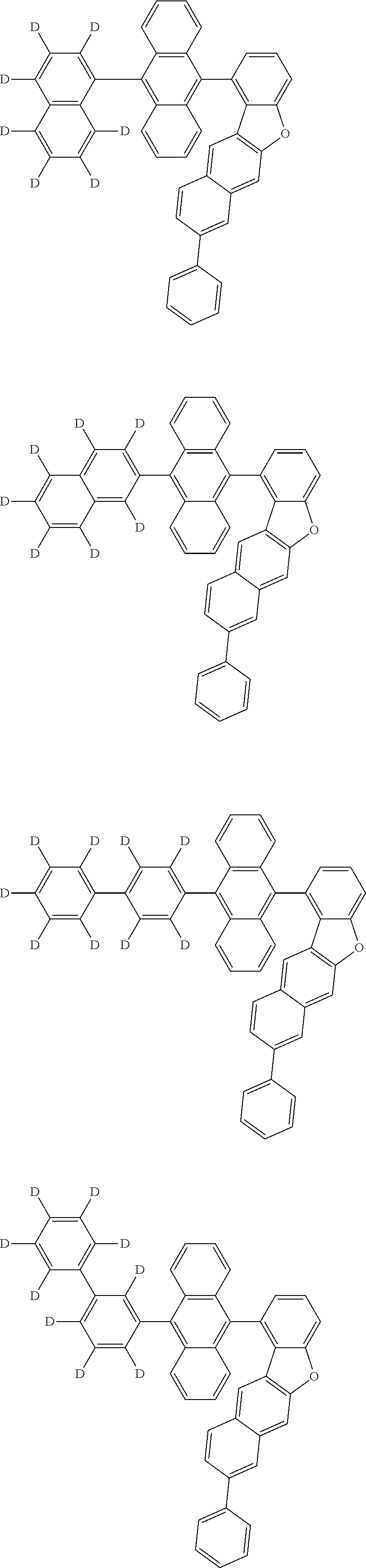

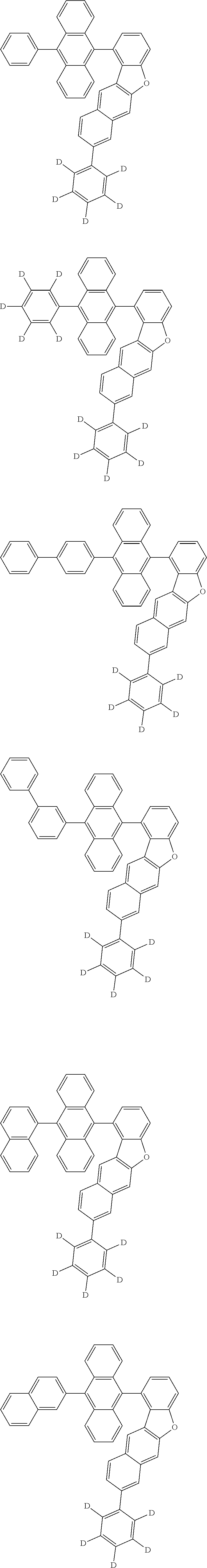

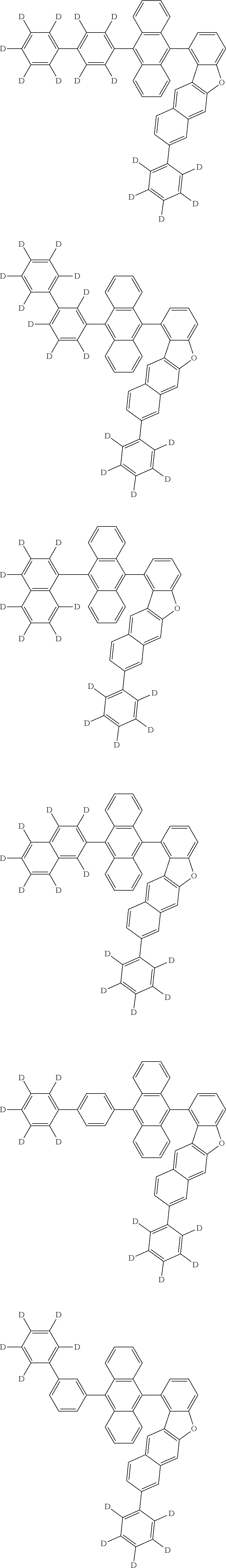

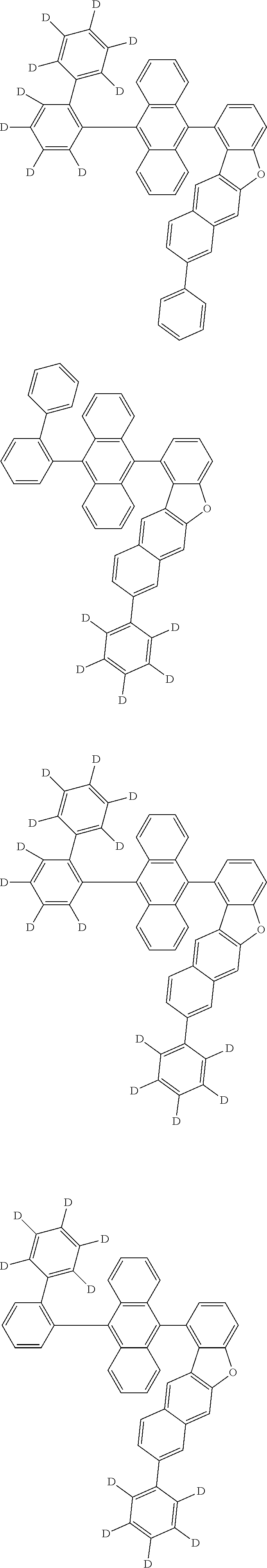

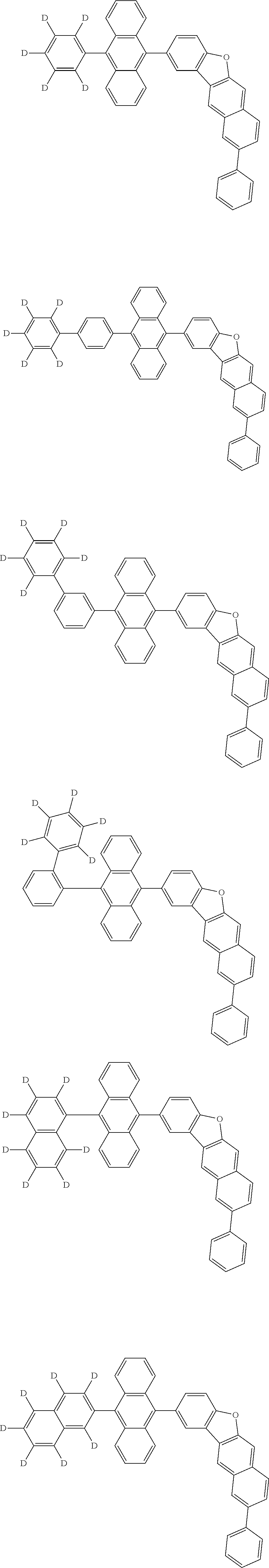

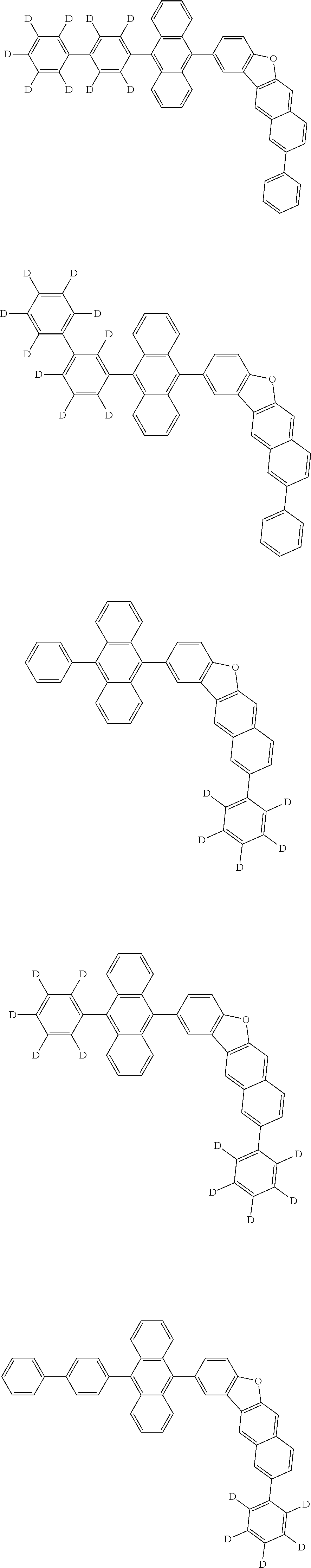

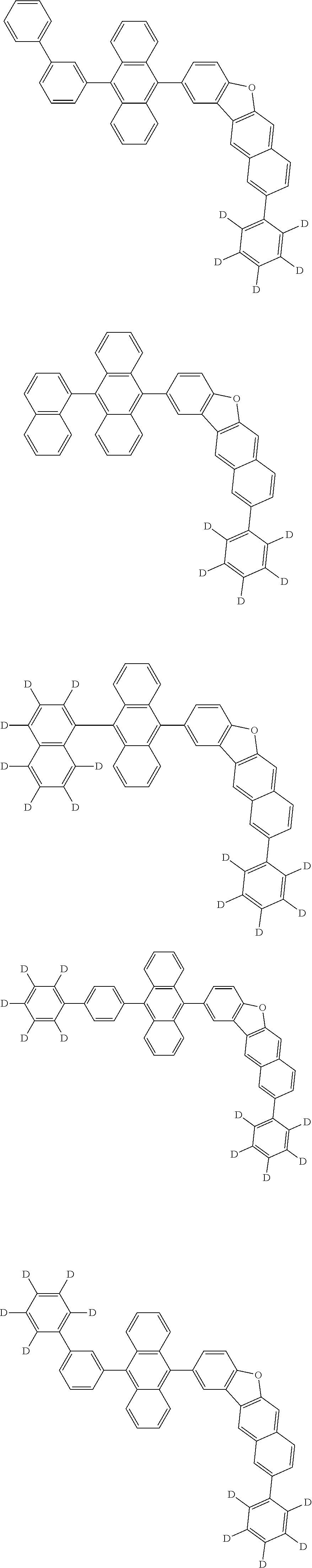

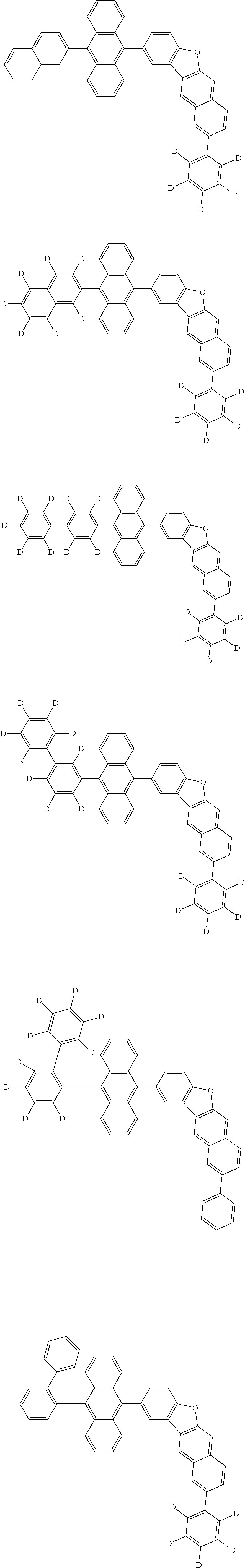

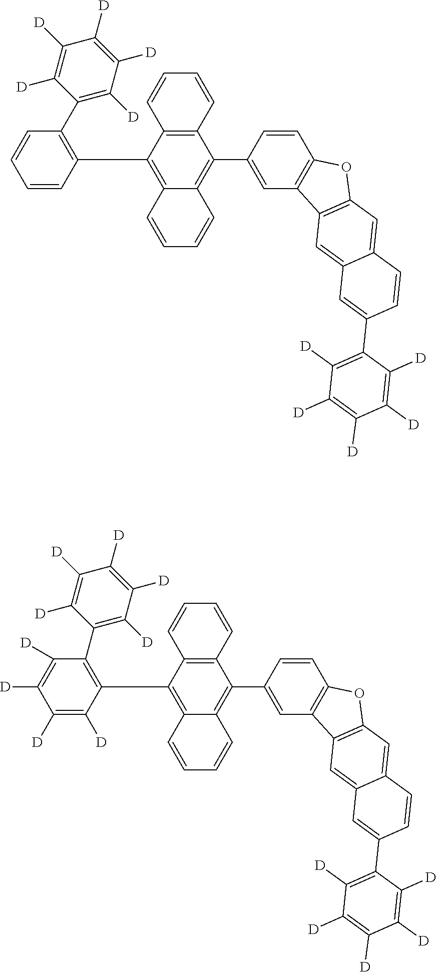 [SMILES: [2H]c1c([2H])c([2H])c(-c2ccc3cc4oc5ccc(-c6c7ccccc7c(-c7ccccc7-c7c([2H])c([2H])c([2H])c([2H])c7[2H])c7ccccc67)cc5c4cc3c2)c([2H])c1[2H] ; [2H]c1c([2H])c([2H])c(-c2ccc3cc4oc5ccc(-c6c7ccccc7c(-c7c([2H])c([2H])c([2H])c([2H])c7-c7c([2H])c([2H])c([2H])c([2H])c7[2H])c7ccccc67)cc5c4cc3c2)c([2H])c1[2H]]

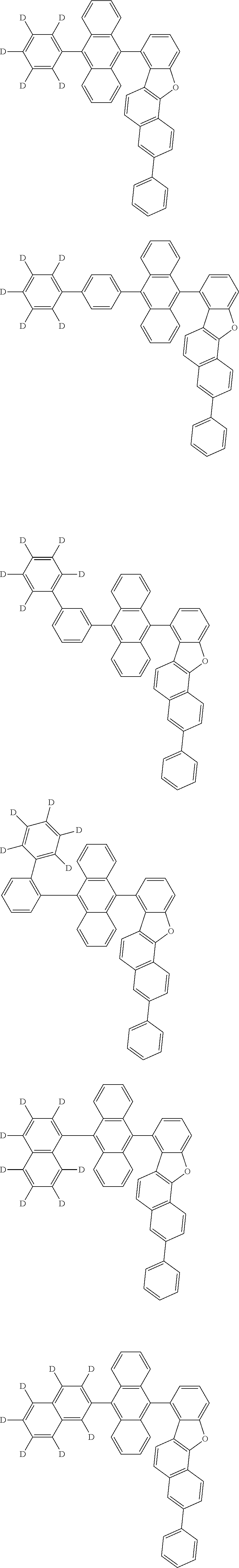

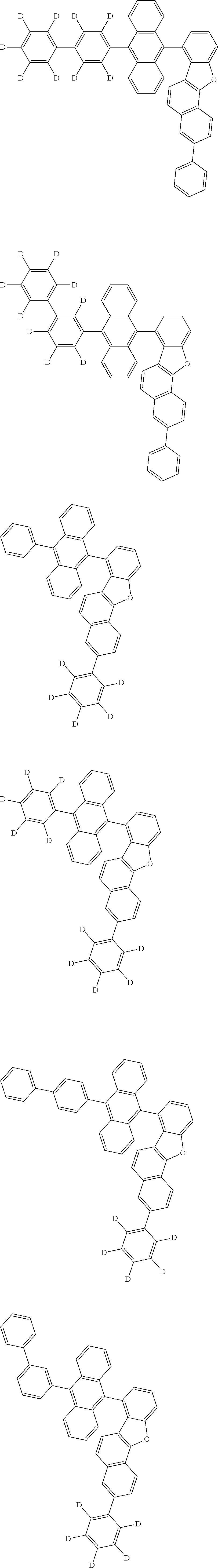

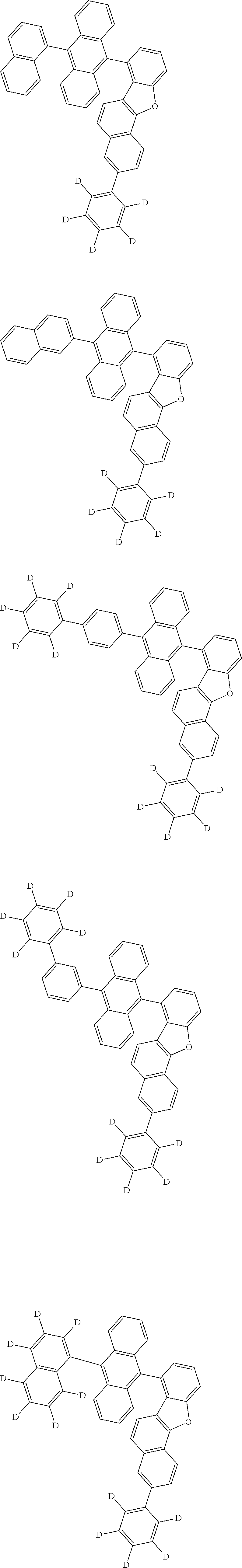

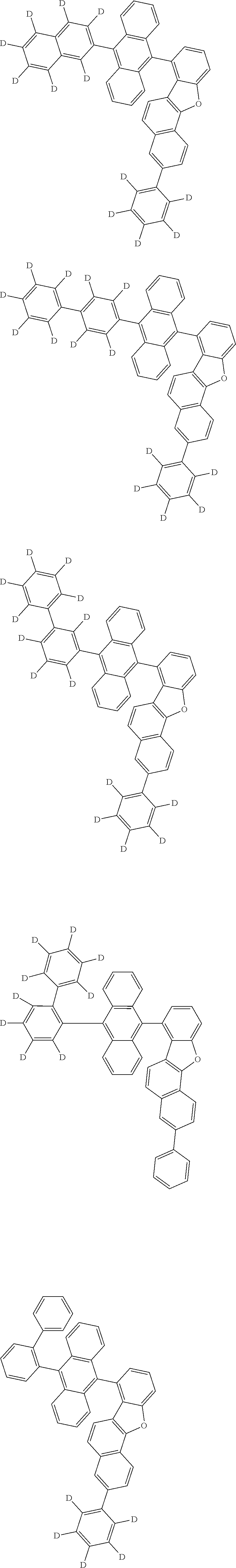

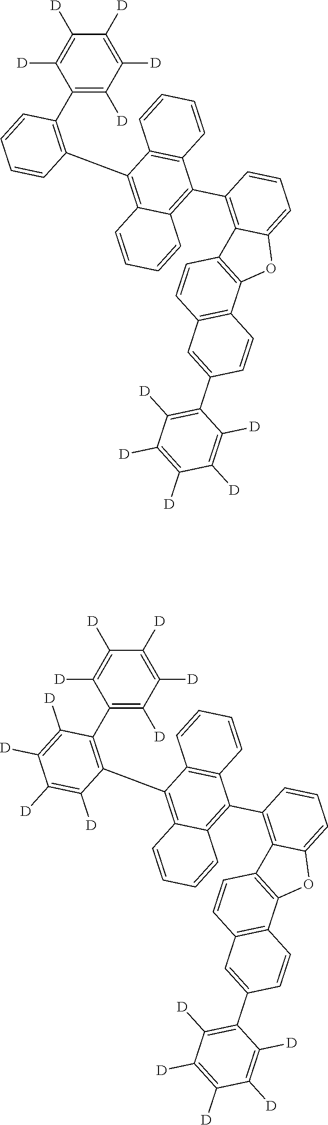 [SMILES: [2H]c1c([2H])c([2H])c(-c2ccc3c(ccc4c3oc3cccc(-c5c6ccccc6c(-c6ccccc6-c6c([2H])c([2H])c([2H])c([2H])c6[2H])c6ccccc56)c34)c2)c([2H])c1[2H] ; [2H]c1c([2H])c([2H])c(-c2ccc3c(ccc4c3oc3cccc(-c5c6ccccc6c(-c6c([2H])c([2H])c([2H])c([2H])c6-c6c([2H])c([2H])c([2H])c([2H])c6[2H])c6ccccc56)c34)c2)c([2H])c1[2H]]

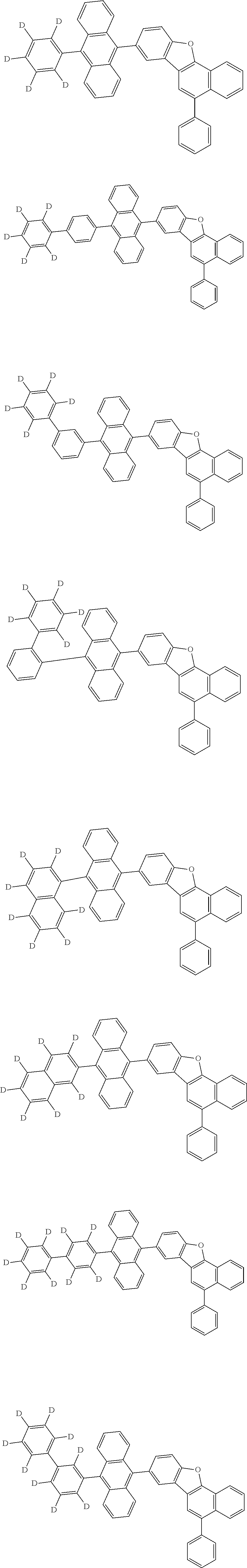

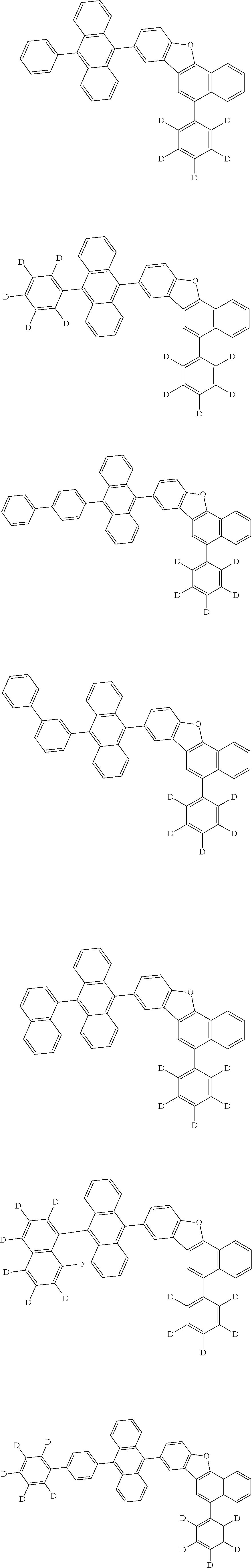

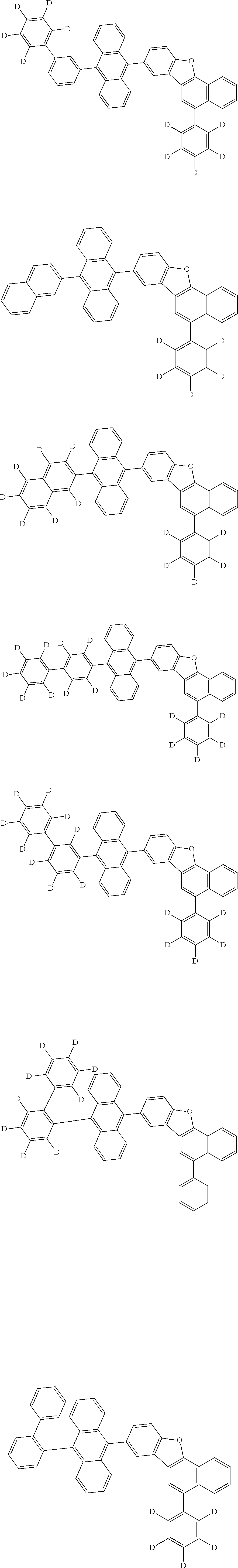

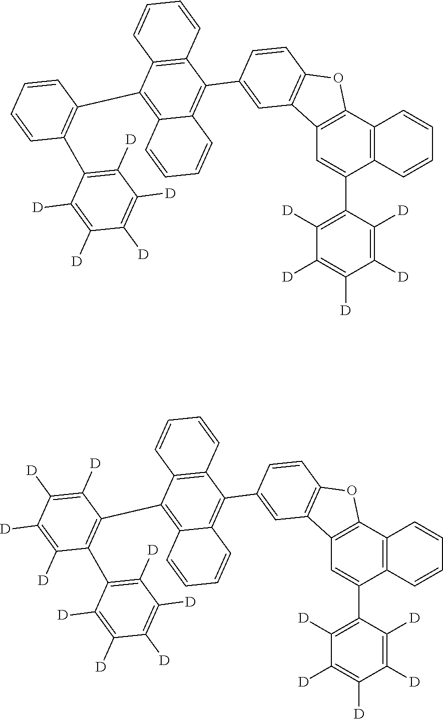 [SMILES: [2H]c1c([2H])c([2H])c(-c2ccccc2-c2c3ccccc3c(-c3ccc4oc5c6ccccc6c(-c6c([2H])c([2H])c([2H])c([2H])c6[2H])cc5c4c3)c3ccccc23)c([2H])c1[2H] ; [2H]c1c([2H])c([2H])c(-c2c([2H])c([2H])c([2H])c([2H])c2-c2c3ccccc3c(-c3ccc4oc5c6ccccc6c(-c6c([2H])c([2H])c([2H])c([2H])c6[2H])cc5c4c3)c3ccccc23)c([2H])c1[2H]]

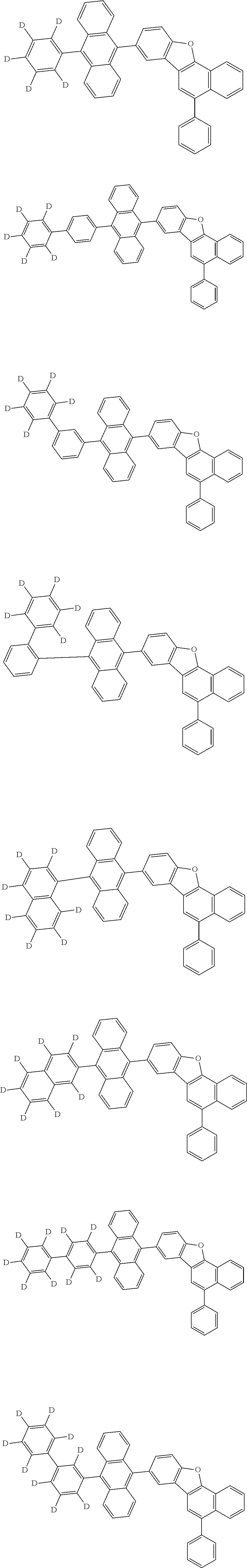

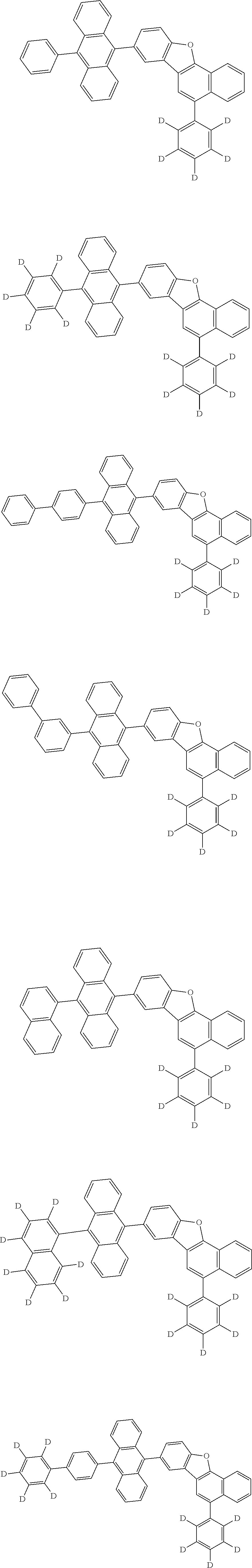

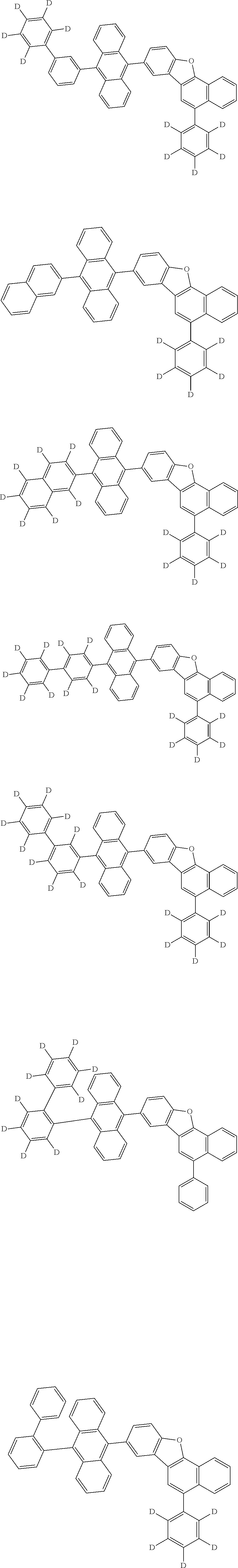

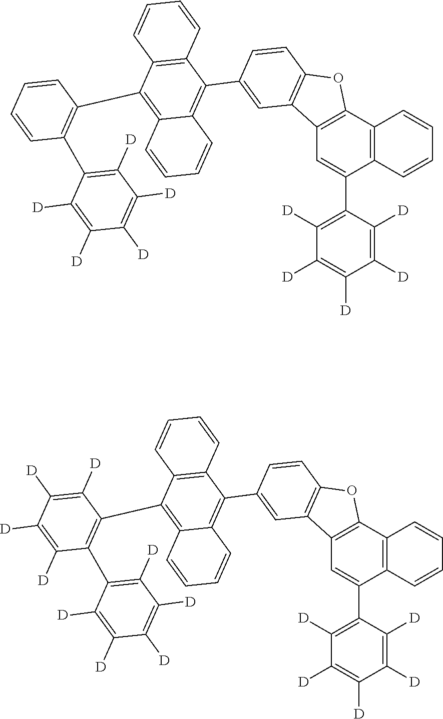 [SMILES: [2H]c1c([2H])c([2H])c(-c2ccccc2-c2c3ccccc3c(-c3ccc4oc5c6ccccc6c(-c6c([2H])c([2H])c([2H])c([2H])c6[2H])cc5c4c3)c3ccccc23)c([2H])c1[2H] ; [2H]c1c([2H])c([2H])c(-c2c([2H])c([2H])c([2H])c([2H])c2-c2c3ccccc3c(-c3ccc4oc5c6ccccc6c(-c6c([2H])c([2H])c([2H])c([2H])c6[2H])cc5c4c3)c3ccccc23)c([2H])c1[2H]]

In one aspect of the present invention, the first compound contains a plurality of compounds represented by the formula (1) and having structures different from each other, and the second compound contains a plurality of compounds represented by the formula (2) and having structures different from each other.

In one aspect of the present invention, the first compound contains the following compounds 1 and 2, and the second compound contains the following compounds 3 and 4 or the following compounds 5 and 6.

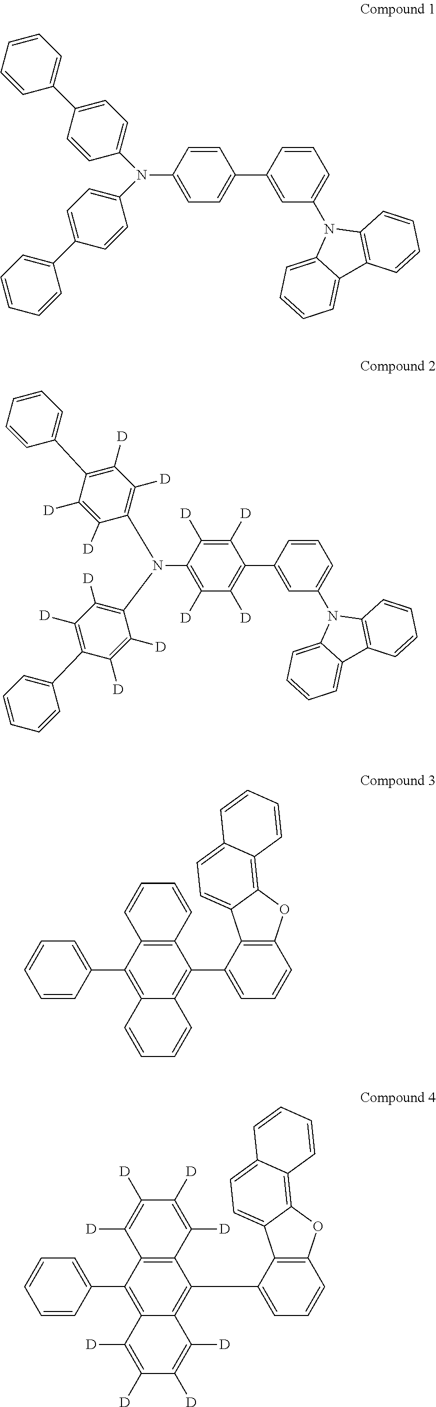 [SMILES: c1ccc(-c2ccc(N(c3ccc(-c4ccccc4)cc3)c3ccc(-c4cccc(-n5c6ccccc6c6ccccc65)c4)cc3)cc2)cc1 ; [2H]c1c([2H])c(N(c2c([2H])c([2H])c(-c3ccccc3)c([2H])c2[2H])c2c([2H])c([2H])c(-c3cccc(-n4c5ccccc5c5ccccc54)c3)c([2H])c2[2H])c([2H])c([2H])c1-c1ccccc1 ; c1ccc(-c2c3ccccc3c(-c3cccc4oc5c6ccccc6ccc5c34)c3ccccc23)cc1 ; [2H]c1c([2H])c([2H])c2c(-c3cccc4oc5c6ccccc6ccc5c34)c3c([2H])c([2H])c([2H])c([2H])c3c(-c3ccccc3)c2c1[2H]]

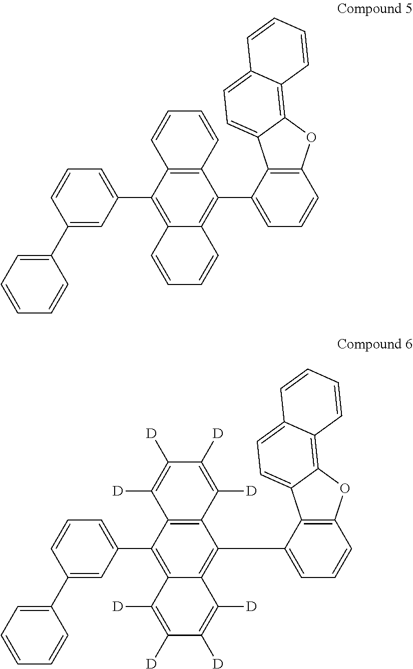 [SMILES: c1ccc(-c2cccc(-c3c4ccccc4c(-c4cccc5oc6c7ccccc7ccc6c45)c4ccccc34)c2)cc1 ; [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H]]

Specific examples of the above groups are as described in the section [Definition] in the description herein.

### <Layer Configuration of Organic EL Element>

As described above, the organic EL element according to the embodiment of the present invention includes: the anode; the cathode; and the organic layer disposed between the anode and the cathode and including the light emitting zone. The organic layer includes the first layer containing the first compound and the second layer containing the second compound. The first layer and the second layer are different layers. The first compound contains 1 mass % or more of the compound having one or more deuterium atoms in the compound represented by the formula (1). The second compound contains 1 mass % or more of the compound having one or more deuterium atoms in the compound represented by the formula (2). In addition, a known material and element configuration in the related art can be applied to the organic EL element as long as the effects of the present invention are not impaired.

Examples of the organic layer containing the first compound and the organic layer containing the second compound include, but are not limited to, a hole transport zone (a hole injection layer, a hole transport layer, an electron blocking layer, an exciton blocking layer, etc.) provided between the anode and the light emitting layer, a light emitting zone (a light emitting layer, a space layer, etc.), and an electron transport zone (an electron injection layer, an electron transport layer, a hole blocking layer, etc.) provided between the cathode and the light emitting layer.

The first compound and the second compound are preferably used as a material for a hole transport zone or a light emitting zone of a fluorescent or phosphorescent EL element.

In a preferred embodiment of the present invention, the organic layer includes a hole transport zone between the anode and the light emitting zone, and the first layer is included in the hole transport zone. In other words, the first compound is preferably used as a material for the hole transport zone.

The hole transport zone includes at least one or more layers having a hole transport function. Examples of the layer constituting the hole transport zone include a hole injection layer, a hole transport layer, an electron blocking layer, and an exciton blocking layer. When the first layer is included in the hole transport zone, the first layer may be a single layer constituting the hole transport zone, or may be at least one layer of a plurality of layers constituting the hole transport zone.

In a preferred embodiment of the present invention, the first layer is the hole transport layer. In other words, the first compound is preferably used as a material for the hole transport layer.

In one embodiment of the present invention, the hole transport zone includes a third layer different from the first layer.

In this case, the third layer may be disposed between the anode and the first layer, and the third layer may not contain the first compound.

In a preferred embodiment of the present invention, as described later, the hole transport layer has a multi-layered structure including two or more layers, the hole transport layer has a two-layered structure including a first hole transport layer (on an anode side) and a second hole transport layer (on a cathode side), the first hole transport layer is the third layer, and the second hole transport layer is the first layer.

In one embodiment of the present invention, no other layers are provided between the first layer and the light emitting zone. In other words, in one embodiment of the present invention, the light emitting zone is in direct contact with the first layer.

In a preferred embodiment of the present invention, the light emitting zone contains the second compound. In other words, the second layer is preferably a layer provided in the light emitting zone, and the second compound is preferably used as the material for the light emitting zone of the fluorescent or phosphorescent EL element.

More preferably the second layer is the light emitting layer, and the second compound is used as a material for the light emitting layer. Even more preferably the second compound is used as a host material for the light emitting layer.

In one embodiment of the present invention, the light emitting zone contains a fluorescent dopant material.

In one embodiment of the present invention, the light emitting zone contains a phosphorescent dopant material.

In a preferred embodiment of the present invention, the hole transport zone includes the first layer, and the light emitting zone includes the second layer.

More preferably the hole transport layer is the first layer and the light emitting layer is the second layer. Even more preferably the second hole transport layer is the first layer and the light emitting layer is the second layer.

The organic EL element may be a fluorescent or phosphorescent light emission type monochromatic light emitting element or a fluorescent and phosphorescent hybrid type white light emitting element, or may be a simple type organic EL element including a single light emitting unit or a tandem type organic EL element including a plurality of light emitting units. Among them, a fluorescent light emission type element is preferred. Here, the “light emitting unit” refers to a minimum unit that includes organic layers, at least one of which is a light emitting layer and emits light by recombination of injected holes and electrons.

For example, as a typical element configuration of the simple type organic EL element, the following element configurations can be exemplified.

### (1) Anode/Light Emitting Unit/Cathode

The light emitting unit may be a laminated type light emitting unit including a plurality of phosphorescent light emitting layers or a plurality of fluorescent light emitting layers, and in this case, a space layer may be provided between the light emitting layers for the purpose of preventing excitons generated in the phosphorescent light emitting layers from diffusing into the fluorescent light emitting layers. A typical layer configuration of a simple type light emitting unit is shown below. The layers in parentheses are optional.
(a) (hole injection layer/) hole transport layer/fluorescent light emitting layer (/electron transport layer/electron injection layer)
(b) (hole injection layer/) hole transport layer/phosphorescent light emitting layer (/electron transport layer/electron injection layer)
(c) (hole injection layer/) hole transport layer/first fluorescent light emitting layer/second fluorescent light emitting layer (/electron transport layer/electron injection layer)
(d) (hole injection layer/) hole transport layer/first phosphorescent light emitting layer/second phosphorescent light emitting layer (/electron transport layer/electron injection layer)
(e) (hole injection layer/) hole transport layer/phosphorescent light emitting layer/space layer/fluorescent light emitting layer (/electron transport layer/electron injection layer)
(f) (hole injection layer/) hole transport layer/first phosphorescent light emitting layer/second phosphorescent light emitting layer/space layer/fluorescent light emitting layer (/electron transport layer/electron injection layer)
(g) (hole injection layer/) hole transport layer/first phosphorescent light emitting layer/space layer/second phosphorescent light emitting layer/space layer/fluorescent light emitting layer (/electron transport layer/electron injection layer)
(h) (hole injection layer/) hole transport layer/phosphorescent light emitting layer/space layer/first fluorescent light emitting layer/second fluorescent light emitting layer (/electron transport layer/electron injection layer)
(i) (hole injection layer/) hole transport layer/electron blocking layer/fluorescent light emitting layer (/electron transport layer/electron injection layer)
(j) (hole injection layer/) hole transport layer/electron blocking layer/phosphorescent light emitting layer (/electron transport layer/electron injection layer)
(k) (hole injection layer/) hole transport layer/exciton blocking layer/fluorescent light emitting layer (/electron transport layer/electron injection layer)
(l) (hole injection layer/) hole transport layer/exciton blocking layer/phosphorescent light emitting layer (/electron transport layer/electron injection layer)
(m) (hole injection layer/) first hole transport layer/second hole transport layer/fluorescent light emitting layer (/electron transport layer/electron injection layer)
(n) (hole injection layer/) first hole transport layer/second hole transport layer/phosphorescent light emitting layer (/electron transport layer/electron injection layer)
(o) (hole injection layer/) first hole transport layer/second hole transport layer/fluorescent light emitting layer/first electron transport layer/second electron transport layer (/electron injection layer)
(p) (hole injection layer/) first hole transport layer/second hole transport layer/phosphorescent light emitting layer/first electron transport layer/second electron transport layer (/electron injection layer)
(q) (hole injection layer/) hole transport layer/fluorescent light emitting layer/hole blocking layer (/electron transport layer/electron injection layer)
(r) (hole injection layer/) hole transport layer/phosphorescent light emitting layer/hole blocking layer (/electron transport layer/electron injection layer)
(s) (hole injection layer/) hole transport layer/fluorescent light emitting layer/exciton blocking layer (/electron transport layer/electron injection layer)
(t) (hole injection layer/) hole transport layer/phosphorescent light emitting layer/exciton blocking layer (/electron transport layer/electron injection layer)

The phosphorescent or fluorescent light emitting layers may exhibit emission colors different from each other. Specifically, in the laminated light emitting unit (f), a layer configuration including (hole injection layer/) hole transport layer/first phosphorescent light emitting layer (red light emission)/second phosphorescent light emitting layer (green light emission)/space layer/fluorescent light emitting layer (blue light emission)/electron transport layer is exemplified.

An electron blocking layer may be appropriately provided between each light emitting layer and the hole transport layer or the space layer. A hole blocking layer may be appropriately provided between each light emitting layer and the electron transport layer. By providing the electron blocking layer or the hole blocking layer, electrons or holes can be confined in the light emitting layer, recombination probability of charges in the light emitting layer can be increased, and luminous efficiency can be improved.

As a typical element configuration of the tandem type organic EL element, the following element configuration can be exemplified.

### (2) Anode/First Light Emitting Unit/Intermediate Layer/Second Light Emitting Unit/Cathode

Here, the first light emitting unit and the second light emitting unit can be independently selected from the light emitting units described above, for example.

The intermediate layer is generally referred to as an intermediate electrode, an intermediate conductive layer, a charge generation layer, an electron removal layer, a connection layer, or an intermediate insulating layer, and may have a known material configuration in which electrons are supplied to the first light emitting unit and holes are supplied to the second light emitting unit.

In the present invention, a host combined with a fluorescent dopant (a fluorescent light emitting material) is referred to as a fluorescent host, and a host combined with a phosphorescent dopant is referred to as a phosphorescent host. The fluorescent host and the phosphorescent host are not distinguished from each other only by a molecular structure. That is, the phosphorescent host means a material for forming a phosphorescent light emitting layer containing a phosphorescent dopant, and does not mean that the phosphorescent host cannot be used as a material for forming a fluorescent light emitting layer. The same applies to the fluorescent host.

Hereinafter, members used in the organic EL element according to the embodiment of the present invention, materials constituting each layer other than the above compounds, and the like will be described.

### (Substrate)

A substrate is used as a support for the organic EL element. As the substrate, for example, a plate made of glass, quartz, plastic, or the like can be used. Alternatively a flexible substrate may be used. Examples of the flexible substrate include a plastic substrate made of a polycarbonate, a polyarylate, a polyethersulfone, a polypropylene, a polyester, polyvinyl fluoride, and polyvinyl chloride. An inorganic vapor deposition film can also be used.

### (Anode)

For the anode formed on the substrate, a metal, an alloy an electrically conductive compound, and a mixture thereof, which have a large work function (specifically 4.0 eV or more), are preferably used. Specific examples thereof include indium tin oxide (ITO), silicon or silicon oxide-containing indium tin oxide, indium zinc oxide, tungsten oxide and zinc oxide-containing indium oxide, and graphene. Other examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), and nitrides of the above metals (for example, titanium nitride).

These materials are generally formed by a sputtering method. For example, the indium zinc oxide can be formed by the sputtering method by using a target obtained by adding 1 wt % to 10 wt % of zinc oxide to indium oxide, and the tungsten oxide and zinc oxide-containing indium oxide can be formed by the sputtering method by using a target obtained by adding 0.5 wt % to 5 wt % of tungsten oxide and 0.1 wt % to 1 wt % of zinc oxide to indium oxide. In addition, a vacuum deposition method, a coating method, an ink jet method, a spin coating method, or the like may be used.

Since the hole injection layer formed in contact with the anode is formed using a material that allows easy hole injection regardless of the work function of the anode, a material (for example, a metal, an alloy an electrically conductive compound, a mixture thereof, and an element belonging to Group 1 or Group 2 in the periodic table) that is generally used as an electrode material can be used.

An element belonging to Group 1 or Group 2 in the periodic table, which is a material having a small work function, that is, an alkali metal such as lithium (Li) and cesium (Cs), an alkaline earth metal such as magnesium (Mg), calcium (Ca), and strontium (Sr), an alloy containing the same (for example, MgAg or AlLi), a rare earth metal such as europium (Eu) and ytterbium (Yb), or an alloy containing the same can be used. When forming the anode using an alkali metal, an alkaline earth metal, or an alloy containing the same, a vacuum deposition method or a sputtering method can be used. Further, when using a silver paste or the like, a coating method, an ink jet method, or the like can be used.

### (Hole Transport Zone)

The hole transport zone includes a hole injection layer, a hole transport layer, an electron blocking layer, and the like. It is preferred that any of these layers contains the first compound, and it is particularly preferred that the hole transport layer contains the first compound.

### (Hole Injection Layer)

The hole injection layer is a layer containing a material having a high hole injecting property (a hole injecting material). A single hole injecting material or a combination of a plurality of hole injecting materials can be used for the hole injection layer.

As the hole injecting material, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, or the like can be used.

Examples of the hole injection layer material also include an aromatic amine compound such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1), which are low-molecular-weight organic compounds.

A polymer compound (an oligomer, a dendrimer, a polymer, etc.) can also be used. Examples thereof include a polymer compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). A polymer compound added with an acid such as poly(3,4-ethylenedioxythiophene)/poly(styrenesulfonic acid) (PEDOT/PSS) or polyaniline/poly(styrenesulfonic acid) (PAni/PSS) can also be used.

Further, it is also preferred to use an acceptor material such as a hexaazatriphenylene (HAT) compound represented by the following formula (K) in combination with another compound.

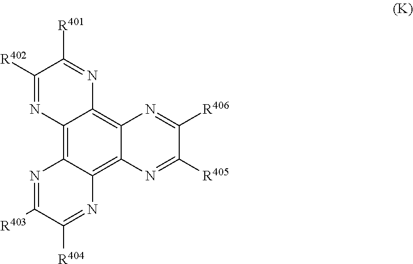

(In the formula (K), R⁴⁰¹ to R⁴⁰⁶ each independently represent a cyano group, —CONH₂, a carboxy group, or —COOR⁴⁰⁷ (R⁴⁰⁷ represents an alkyl group having 1 to 20 carbon atoms or a cycloalkyl group having 3 to 20 carbon atoms). Adjacent two selected from R⁴⁰¹ and R⁴⁰², R⁴⁰³ and R⁴⁰⁴, and R⁴⁰⁵ and R⁴⁰⁶ may be bonded to each other to form a group represented by —CO—O—CO—.)

Examples of R⁴⁰⁷ include a methyl group, an ethyl group, an n-propyl group, an isopropyl group, an n-butyl group, an isobutyl group, a t-butyl group, a cyclopentyl group, and a cyclohexyl group.

### (Hole Transport Layer)

The hole transport layer is a layer containing a material having a high hole transporting property (a hole transporting material). A single hole transporting material or a combination of a plurality of hole transporting materials can be used. As the hole transporting material, for example, an aromatic amine compound, a carbazole derivative, and an anthracene derivative can be used.

Examples of the aromatic amine compound include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The aromatic amine compound has a hole mobility of 10⁻⁶ cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

A polymer compound such as poly(N-vinylcarbazole) (abbreviation: PVK) and poly(4-vinyltriphenylamine) (abbreviation: PVTPA) can also be used.

A compound other than the above may be used as long as the compound has a hole transporting property higher than an electron transporting property.

The hole transport layer may have a single-layered structure or a multi-layered structure including two or more layers. For example, the hole transport layer may be a two-layered structure including a first hole transport layer (on the anode side) and a second hole transport layer (on the cathode side). The above hole transport layers are each formed of the above hole transporting material.

In the hole transport layer having a two-layered structure, the first compound may be contained in one or both of the first hole transport layer and the second hole transport layer. However, the first compound contained in the first hole transport layer and the first compound contained in the second hole transport layer are different from each other.

In a preferred aspect of the present invention, the first compound is contained in the second hole transport layer. In another aspect, the first compound is contained in the first hole transport layer and the second hole transport layer. In still another aspect, the first compound is contained in the first hole transport layer.

In the organic EL element, when the hole transport layer contains the first compound, a content thereof is preferably 10 mass % or more, more preferably 30 mass % or more, and even more preferably 50 mass % or more. An upper limit of the content of the first compound in the hole transport layer is 100 mass %. In other words, when the hole transport layer contains the first compound, the content thereof is preferably 50 mass % to 100 mass %. The present embodiment does not exclude the case where the hole transport layer contains a material other than the first compound.

### (Light Emitting Zone)

The light emitting zone includes a single light emitting layer, a plurality of light emitting layers, a space layer disposed between the plurality of light emitting layers and each light emitting layer, and the like. It is preferred that any of these layers contains the second compound, and it is particularly preferred that the light emitting layer contains the second compound.

### (Dopant Material for Light Emitting Layer)

The light emitting layer is a layer containing a material (a dopant material) having a high light emitting property and various materials can be used. For example, a fluorescent light emitting material or a phosphorescent light emitting material can be used as the dopant material. The fluorescent light emitting material is a compound that emits light from a singlet excited state, and the phosphorescent light emitting material is a compound that emits light from a triplet excited state.

As a blue fluorescent light emitting material that can be used in the light emitting layer, a pyrene derivative, a styrylamine derivative, a chrysene derivative, a fluoranthene derivative, a fluorene derivative, a diamine derivative, a triarylamine derivative, or the like can be used. Specific examples thereof include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), and 4-(10-phenyl-9-anthryl)-4′-(9-phenyl-9H-carbazol-3-yl)triphenylamine (abbreviation: PCBAPA).

As a green fluorescent light emitting material that can be used in the light emitting layer, an aromatic amine derivative or the like can be used. Specific examples thereof include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazol-9-yl)phenyl]-N-phenylanthracen-2-amine (abbreviation: 2YGABPhA), and N,N,9-triphenylanthracen-9-amine (abbreviation: DPhAPhA).

As a red fluorescent light emitting material that can be used in the light emitting layer, a tetracene derivative, a diamine derivative, or the like can be used. Specific examples thereof include N,N,N′,N′-tetrakis(4-methylphenyl)tetracene-5,11-diamine (abbreviation: p-mPhTD), and 7,14-diphenyl-N,N,N′,N′-tetrakis(4-methylphenyl)acenaphtho[1,2-a]fluoranthene-3,10-diamine (abbreviation: p-mPhAFD).

As a blue phosphorescent light emitting material that can be used in the light emitting layer, a metal complex such as an iridium complex, an osmium complex, or a platinum complex can be used. Specific examples thereof include bis[2-(4′,6′-difluorophenyl)pyridinato-N,C2′]iridium (III) tetrakis(1-pyrazolyl)borate (abbreviation: FIr6), bis[2-(4′,6′-difluorophenyl)pyridinato-N,C2′]iridium (III) picolinate (abbreviation: FIrpic), bis[2-(3′,5′bistrifluoromethylphenyl)pyridinato-N, C2′]iridium (III) picolinate (abbreviation: Ir(CF3ppy)2(pic)), and bis[2-(4′,6′-difluorophenyl)pyridinato-N,C2′]iridium (III) acetylacetonate (abbreviation: FIracac).

As a green phosphorescent light emitting material that can be used in the light emitting layer, an iridium complex or the like can be used. Examples thereof include tris(2-phenylpyridinato-N,C2′)iridium (III) (abbreviation: Ir(ppy)3), bis(2-phenylpyridinato-N,C2′)iridium (III) acetylacetonate (abbreviation: Ir(ppy)2(acac)), bis(1,2-diphenyl-1H-benzimidazolato)iridium (III) acetylacetonate (abbreviation: Ir(pbi)2(acac)), and bis(benzo[h]quinolinato)iridium (III) acetylacetonate (abbreviation: Ir(bzq)2(acac)).

As a red phosphorescent light emitting material that can be used in the light emitting layer, a metal complex such as an iridium complex, a platinum complex, a terbium complex, or a europium complex can be used. Specific examples thereof include an organometallic complex such as bis[2-(2′-benzo[4,5-a]thienyl)pyridinato-N,C3′]iridium (III) acetylacetonate (abbreviation: Ir(btp)2(acac)), bis(1-phenylisoquinolinato-N,C2′)iridium (III) acetylacetonate (abbreviation: Ir(piq)2(acac)), (acetylacetonato)bis[2,3-bis(4-fluorophenyl)quinoxalinatoliridium (III) (abbreviation: Ir(Fdpq)2(acac)), and 2,3,7,8,12,13,17,18-octaethyl-21H,23H-porphyrin platinum (II) (abbreviation: PtOEP).

A rare earth metal complex such as tris(acetylacetonate)(monophenanthroline)terbium (III) (abbreviation: Tb(acac)3(Phen)), tris(1,3-diphenyl-1,3-propanedionate)(monophenanthroline)europium (III) (abbreviation: Eu(DBM)3(Phen)), and tris[1-(2-thenoyl)-3,3,3-trifluoroacetonato](monophenanthroline)europium (III) (abbreviation: Eu(TTA)3(Phen)) can be used as a phosphorescent light emitting material because of emitting light from rare earth metal ions (electron transition between different multiplicities).

### (Host Material for Light Emitting Layer)

The light emitting layer may be obtained by dispersing the above dopant material in another material (a host material). As the host material, it is preferred to use a material having a lowest unoccupied molecular orbital level (LUMO level) higher than that of the dopant material and a highest occupied molecular orbit level (HOMO level) lower than that of the dopant material. In a preferred embodiment of the present invention, the second compound is used as the host material for the light emitting layer.

As the host material, use can be made of:
(1) a metal complex such as an aluminum complex, a beryllium complex, or a zinc complex,
(2) a heterocyclic compound such as an oxadiazole derivative, a benzimidazole derivative, or a phenanthroline derivative,
(3) a condensed aromatic compound such as a carbazole derivative, an anthracene derivative, a phenanthrene derivative, a pyrene derivative, or a chrysene derivative, and
(4) an aromatic amine compound such as a triarylamine derivative or a condensed polycyclic aromatic amine derivative.

For example, use can be made of:
a metal complex such as tris(8-quinolinolato)aluminum (III) (abbreviation: Alq), tris(4-methyl-8-quinolinolato)aluminum (III) (abbreviation: Almq3), bis(10-hydroxybenzo[h]quinolinato)beryllium (II) (abbreviation: BeBq2), bis(2-methyl-8-quinolinolato)(4-phenylphenolato)aluminum (III) (abbreviation: BAlq), bis(8-quinolinolato)zinc (II) (abbreviation: Znq), bis[2-(2-benzoxazolyl)phenolato]zinc (II) (abbreviation: ZnPBO), and bis[2-(2-benzothiazolyl)phenolato]zinc (II) (abbreviation: ZnBTZ);
a heterocyclic compound such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), and bathocuproine (abbreviation: BCP);
a condensed aromatic compound such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripylene (abbreviation: TPB3), 9,10-diphenylanthracene (abbreviation: DPAnth), and 6,12-dimethoxy-5,11-diphenylchrysene; and
an aromatic amine compound such as N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB). A plurality of host materials may be used.

Particularly in the case of a blue fluorescent element, it is preferred to use the following anthracene compound as the host material.

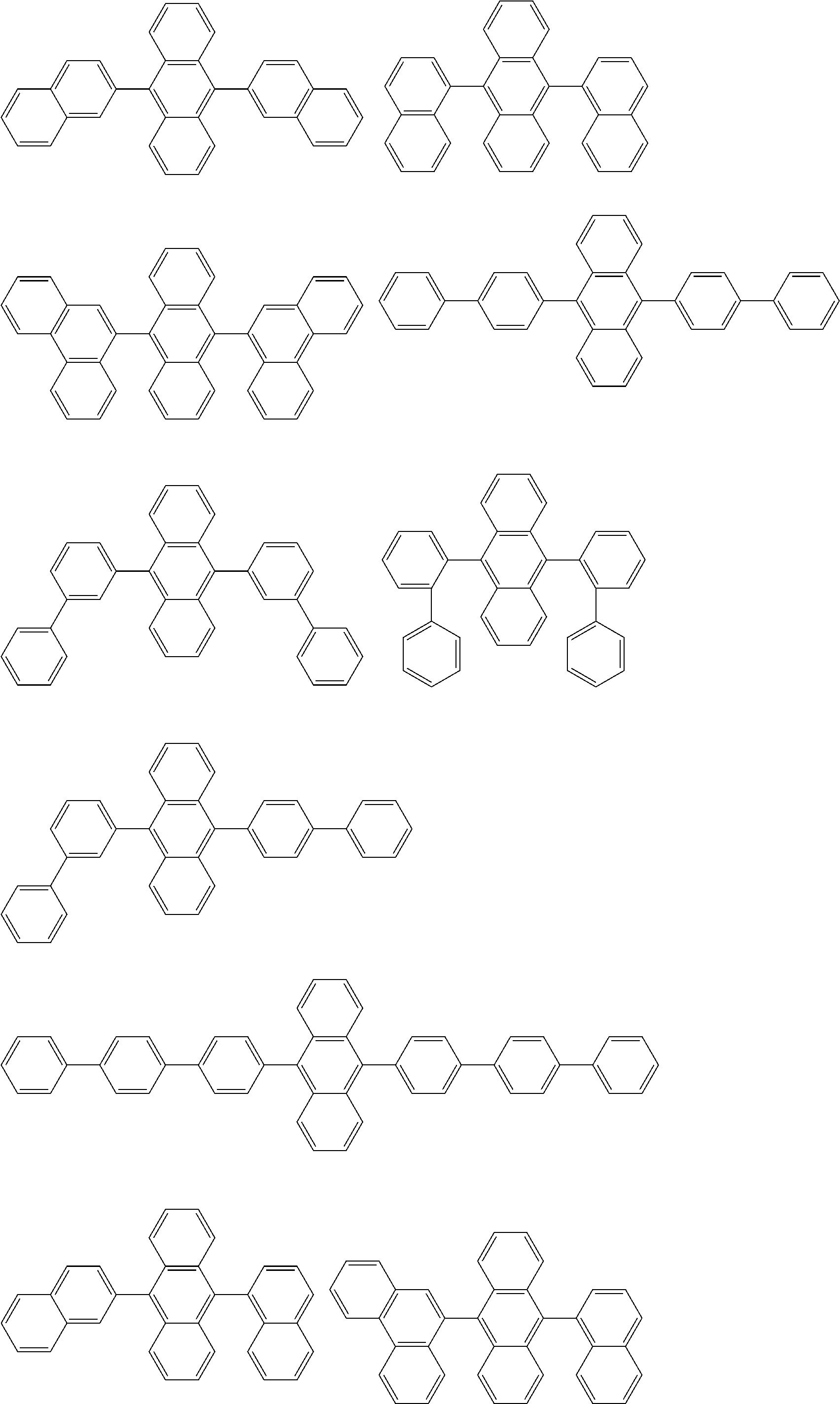

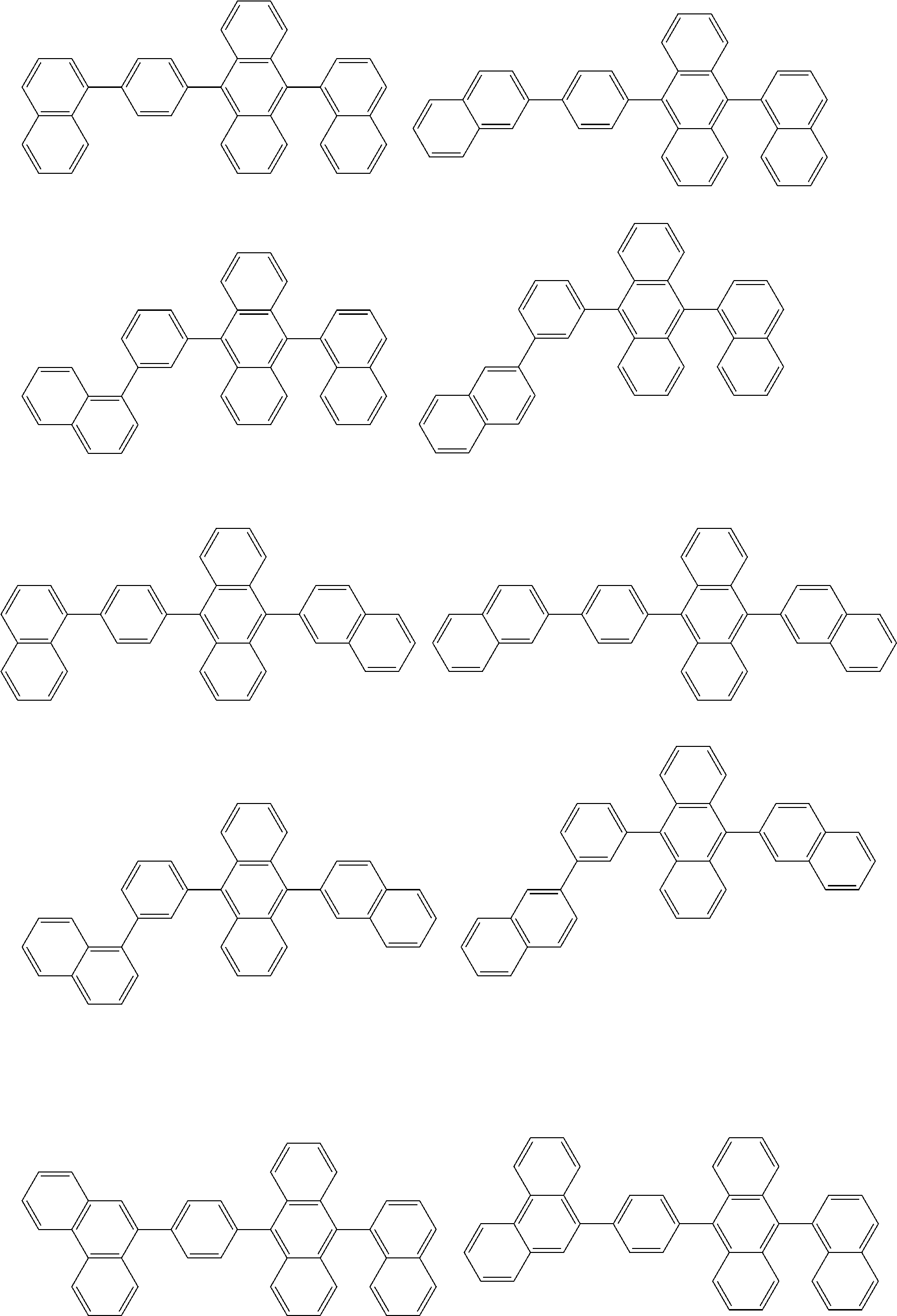

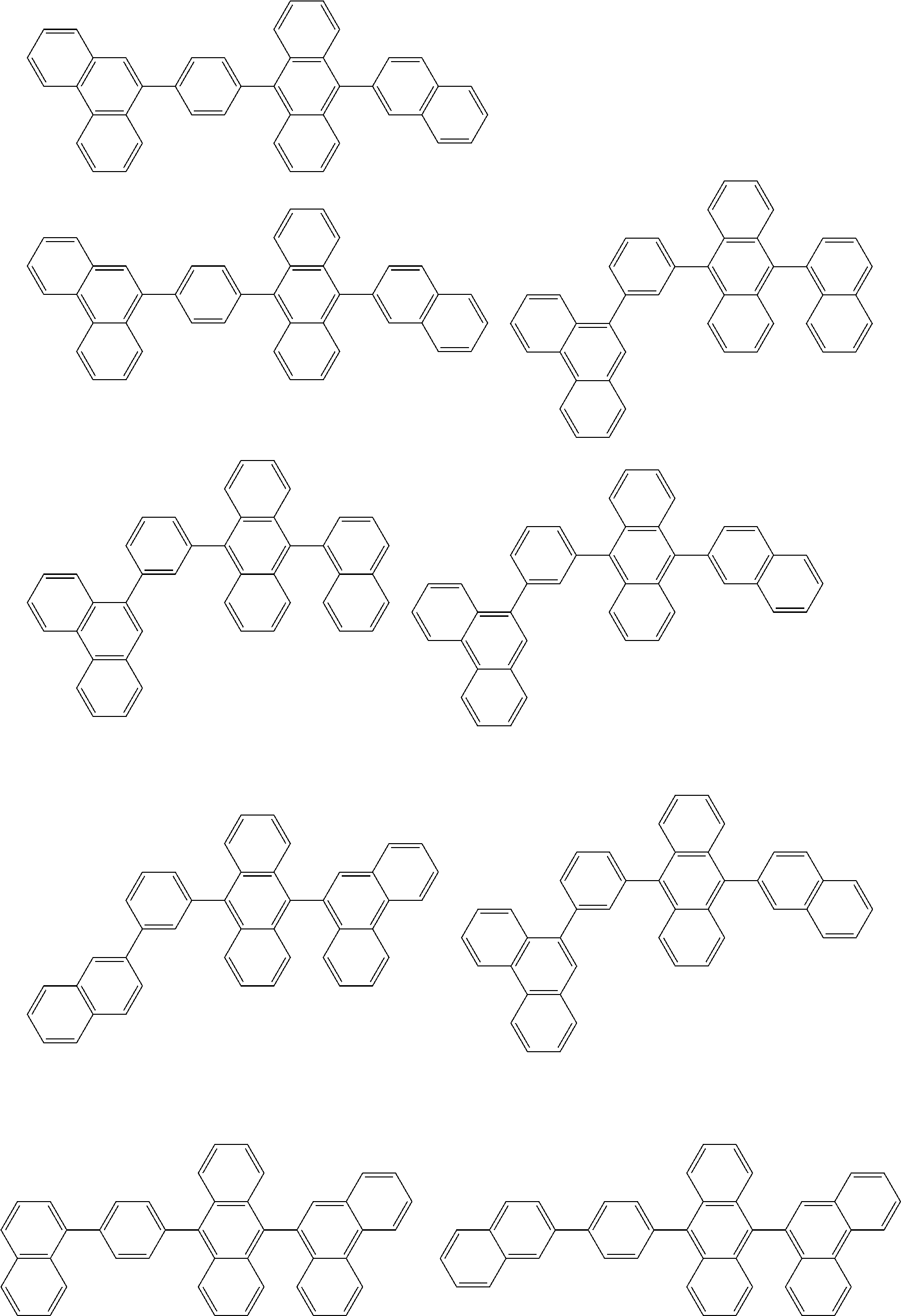

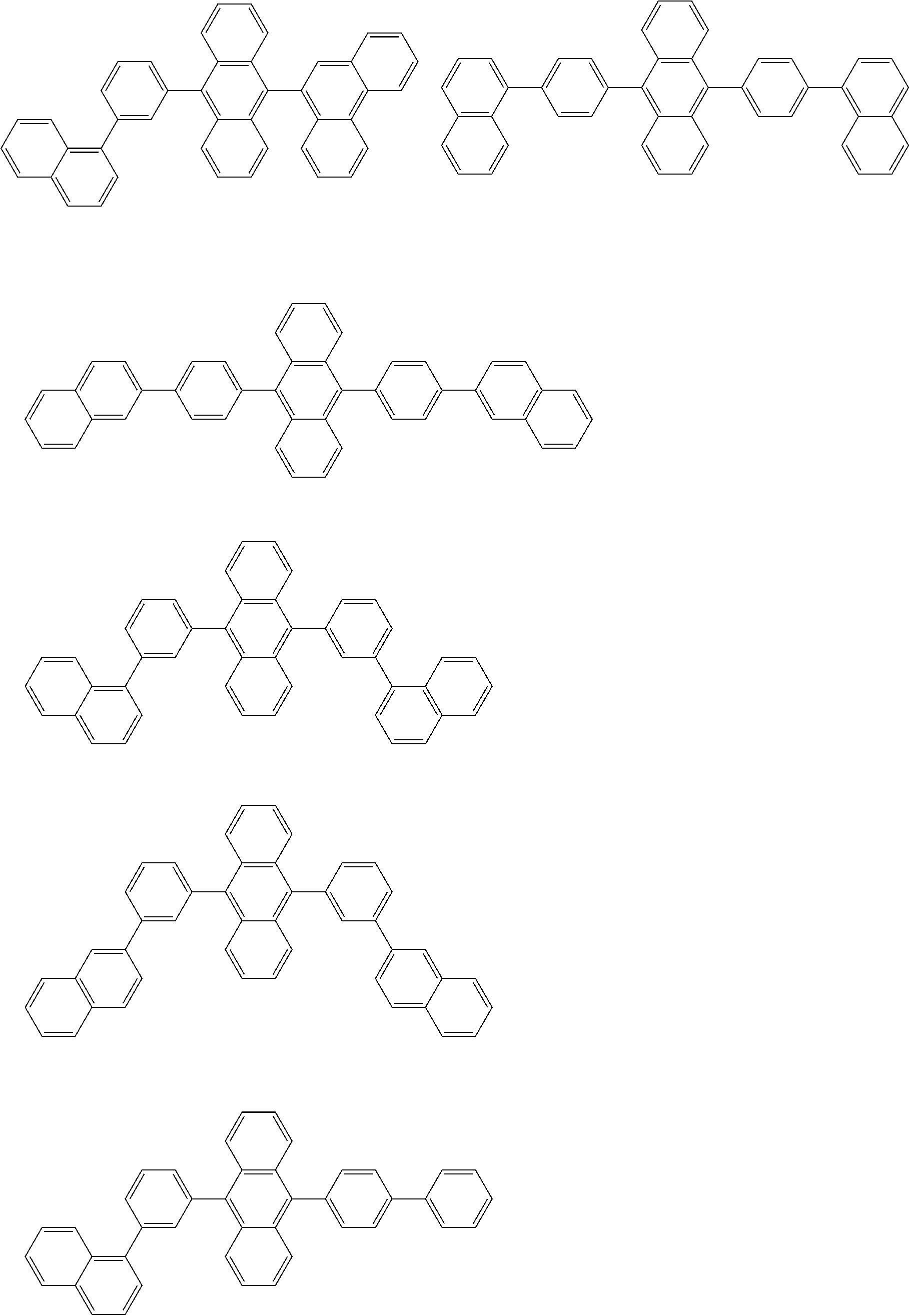

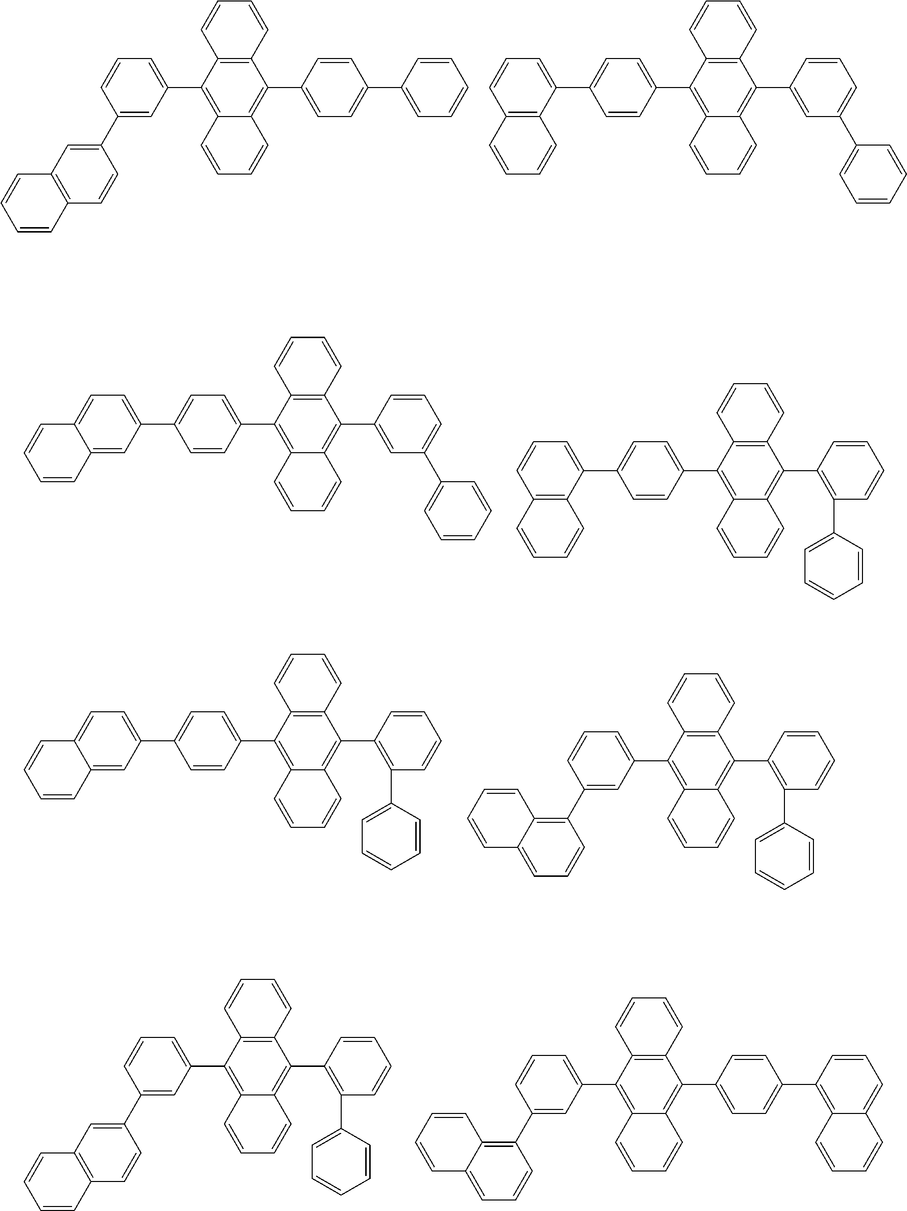

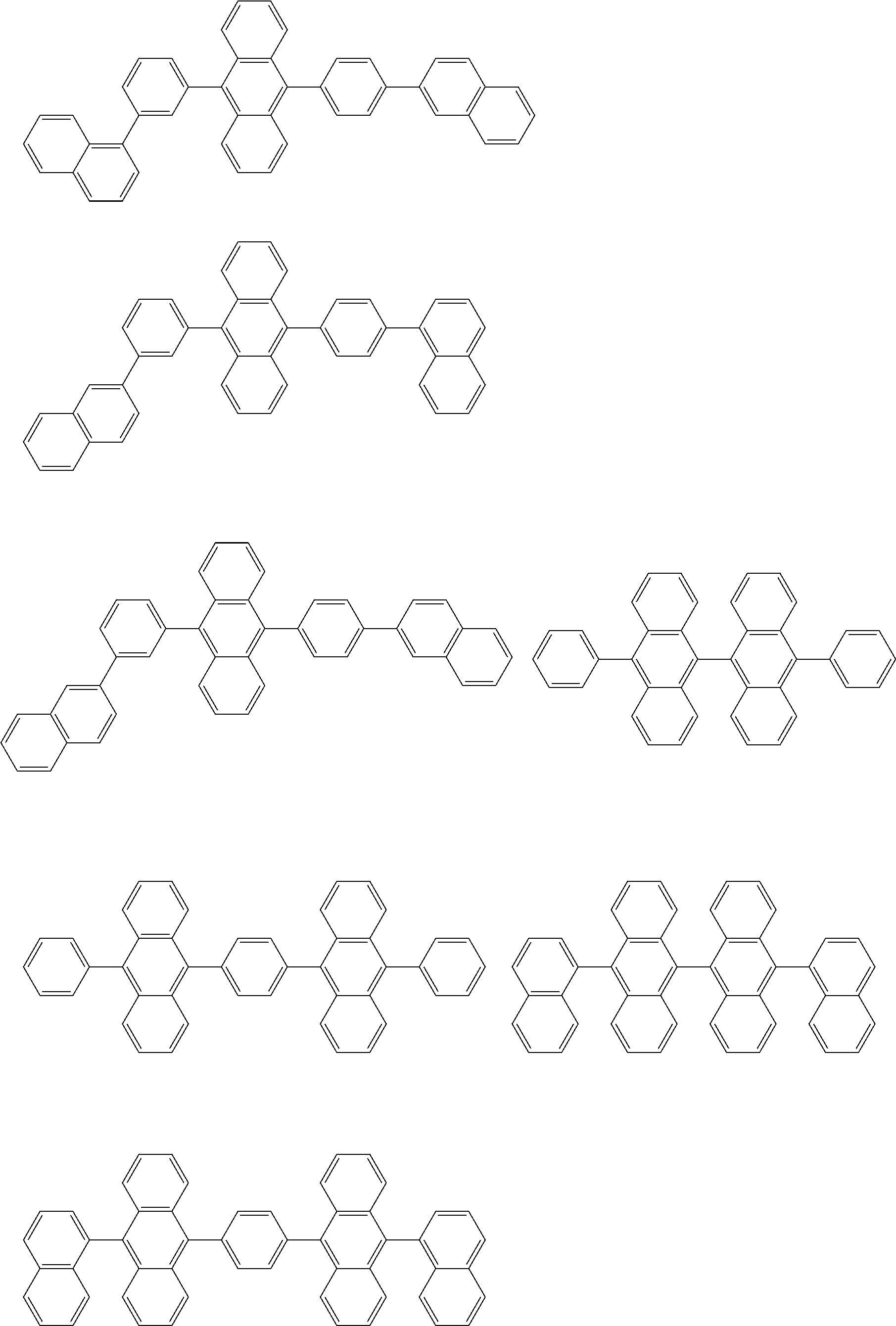

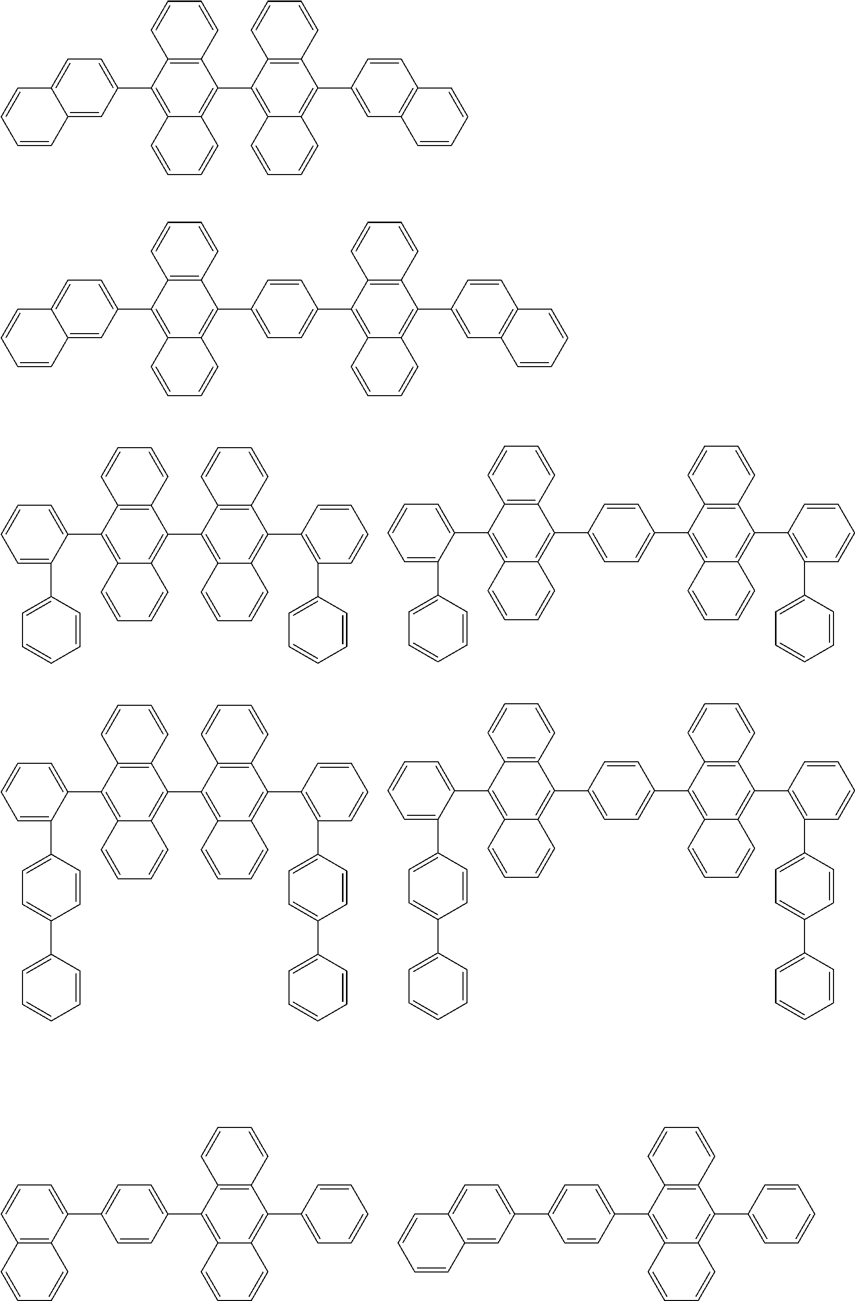

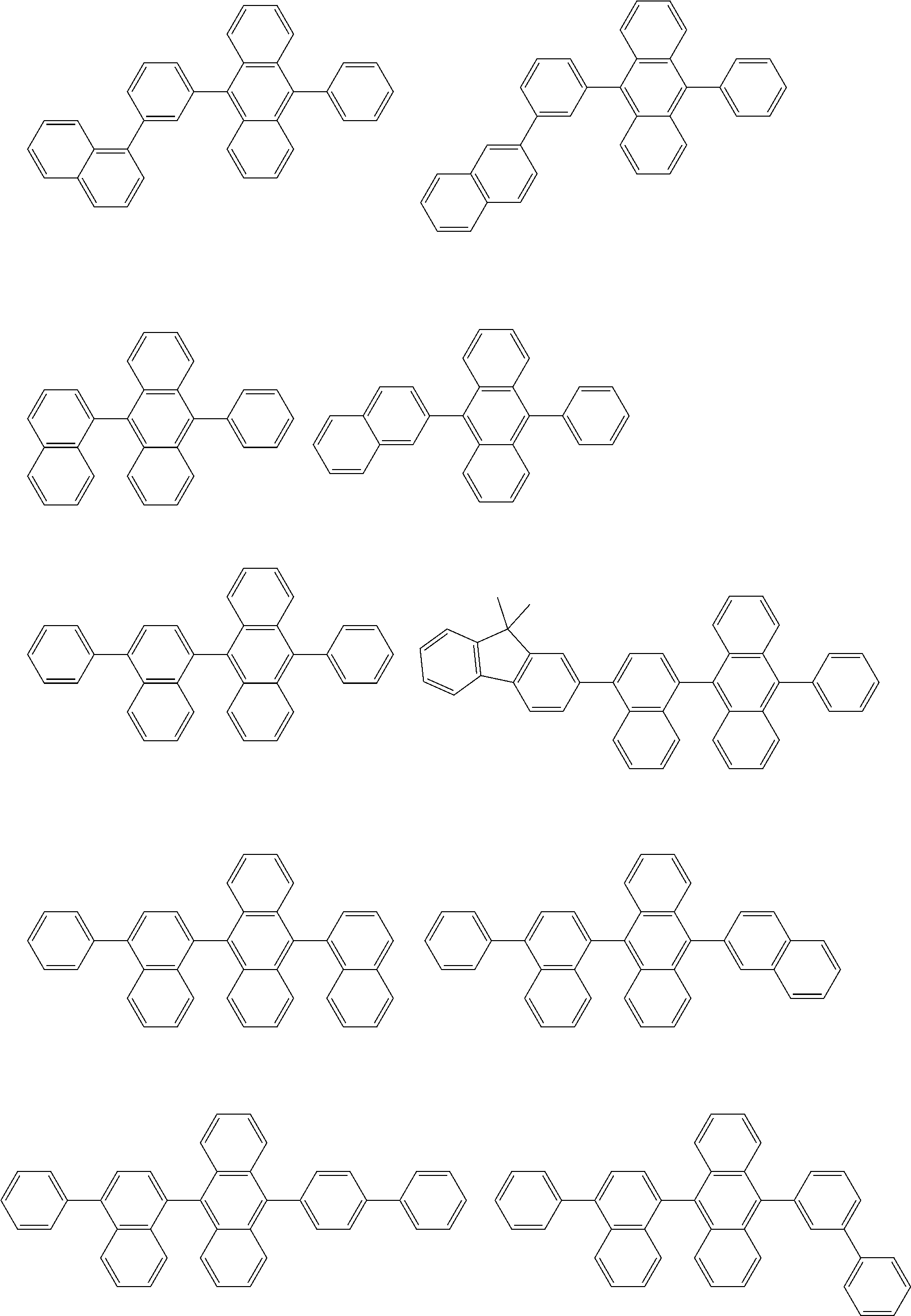

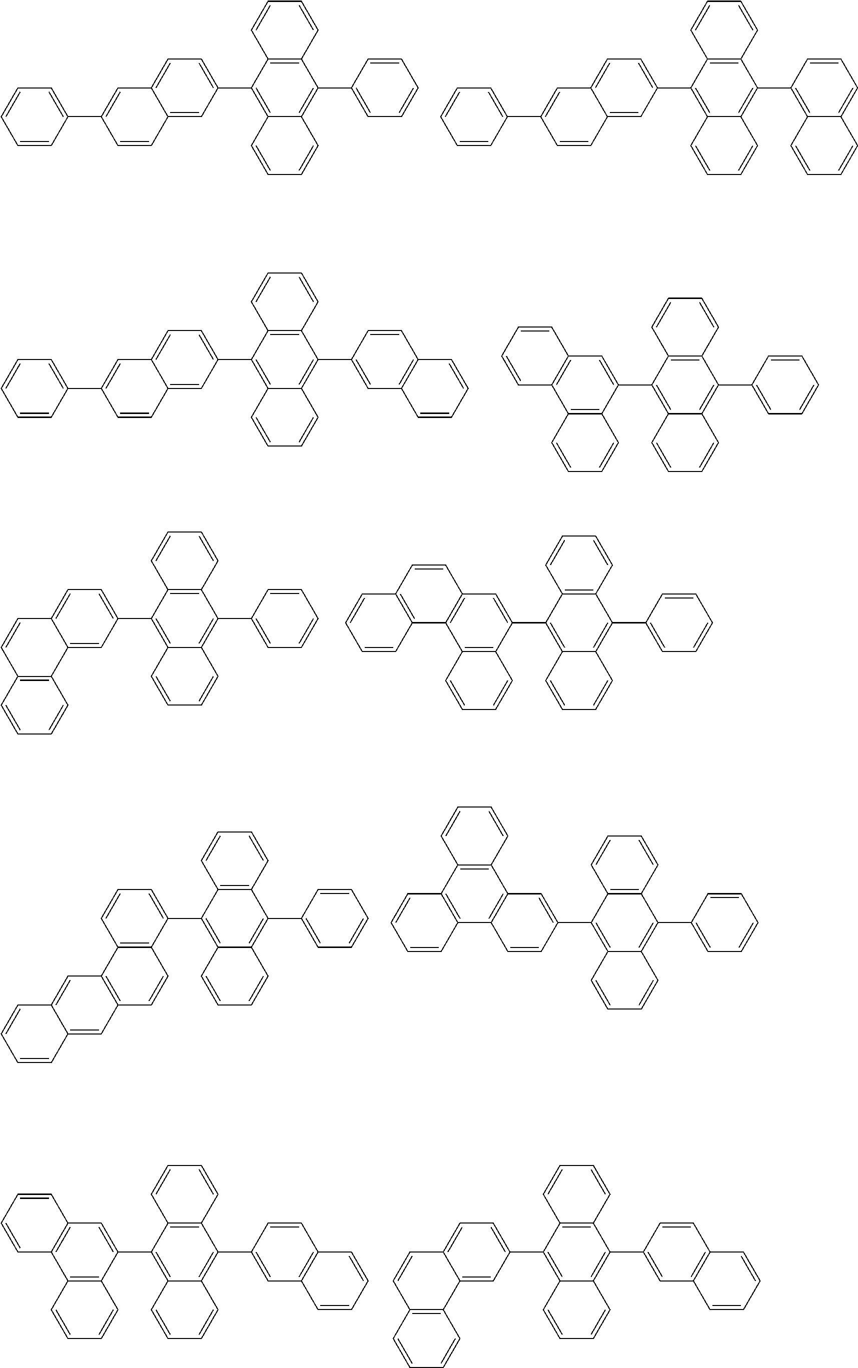

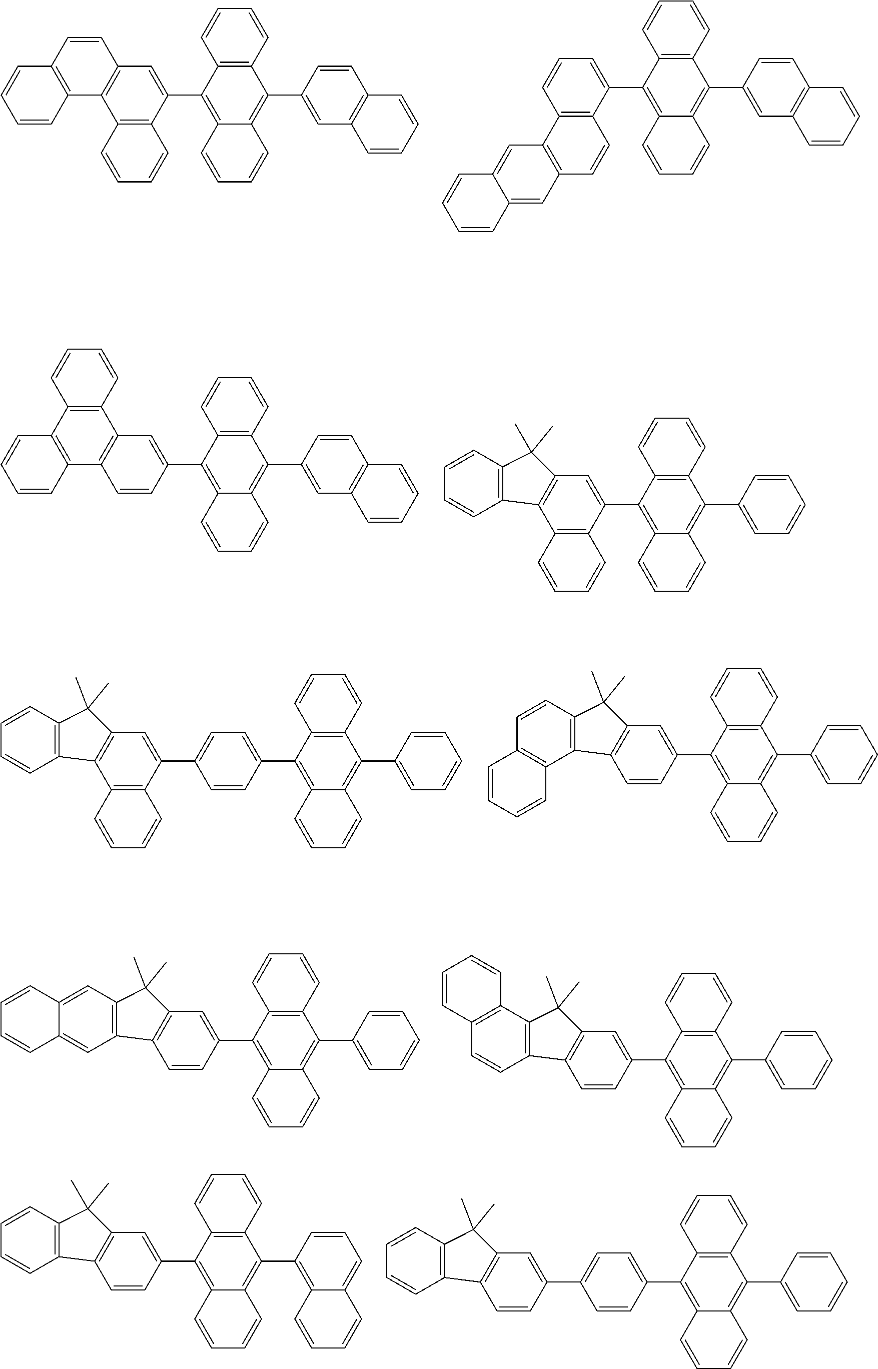

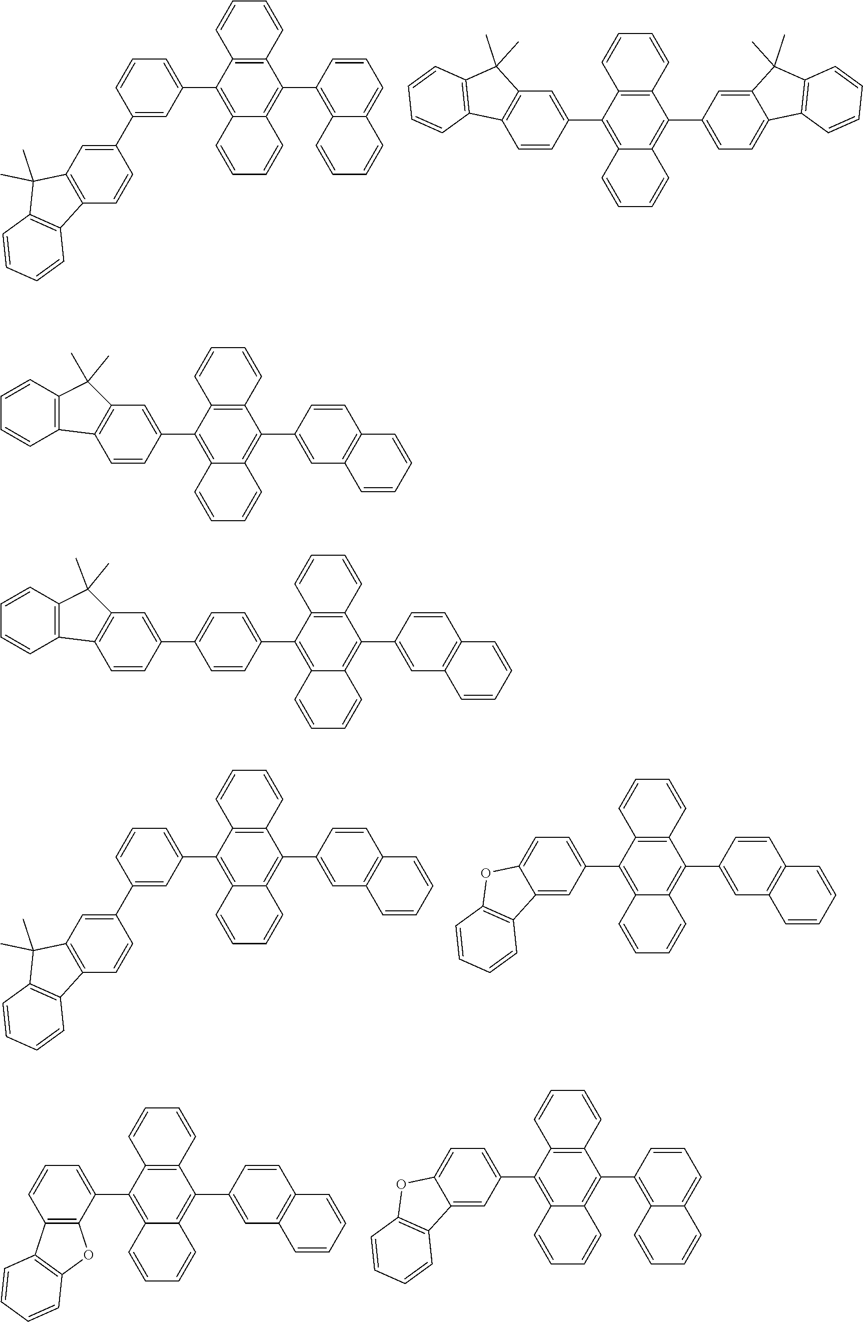

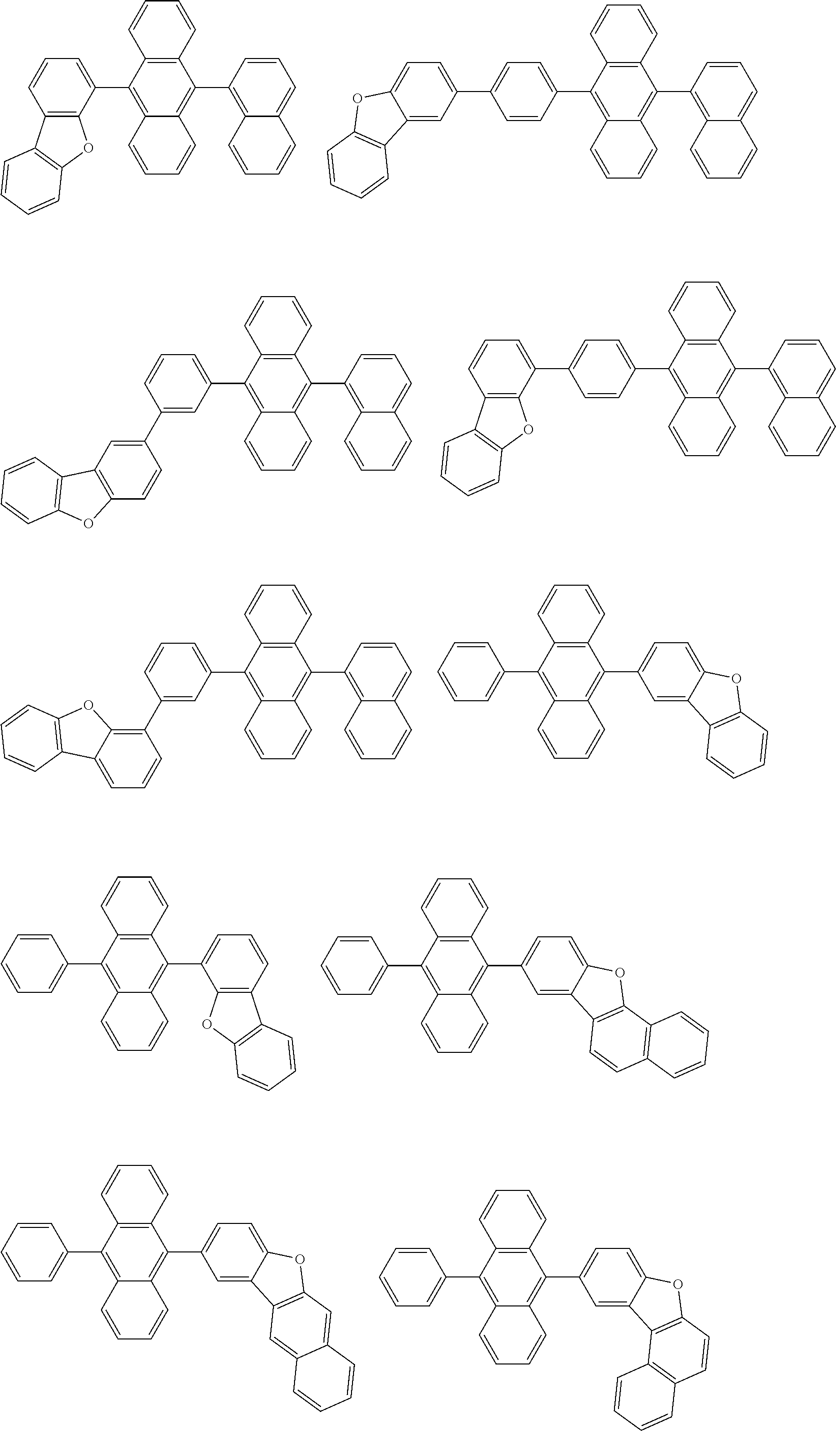

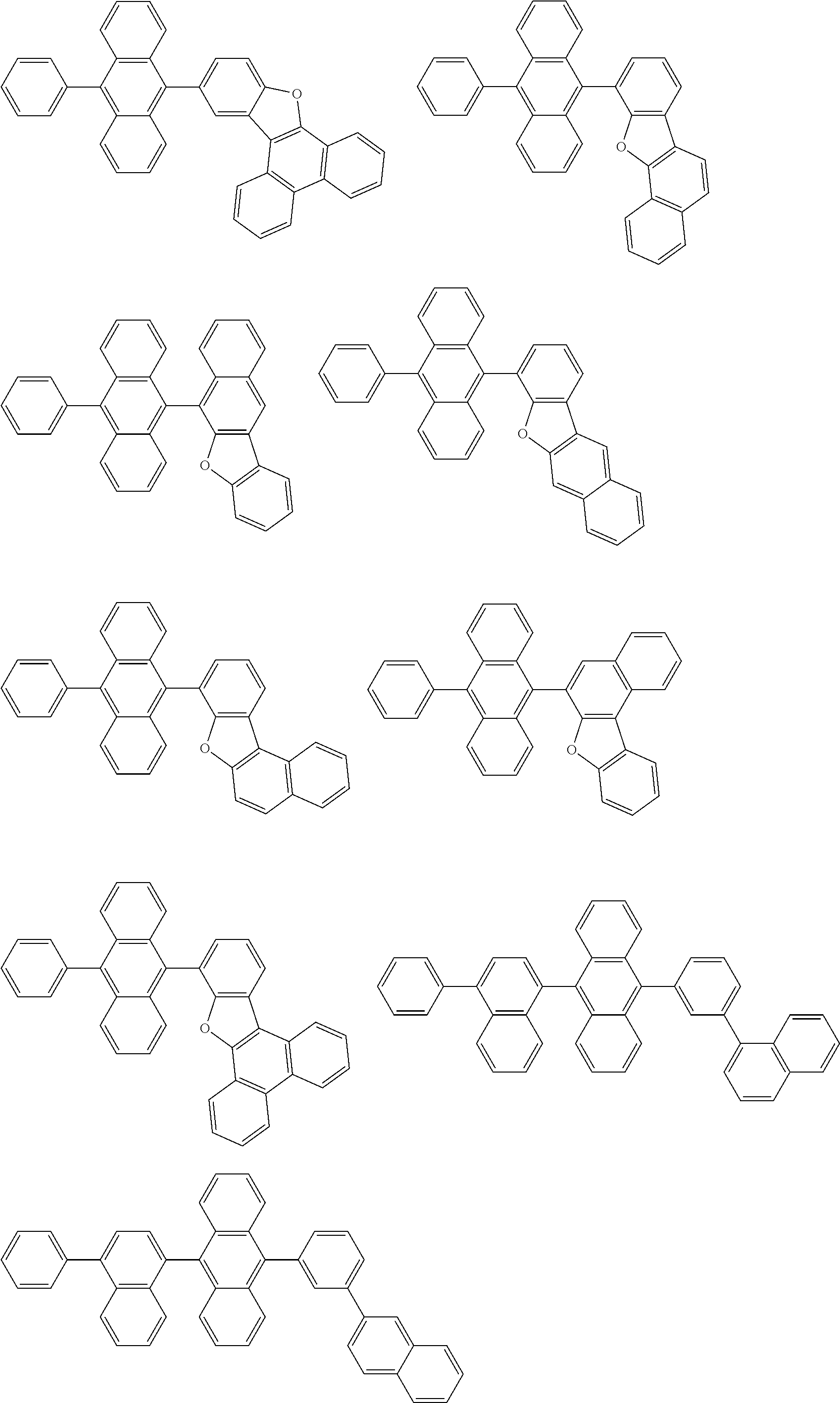

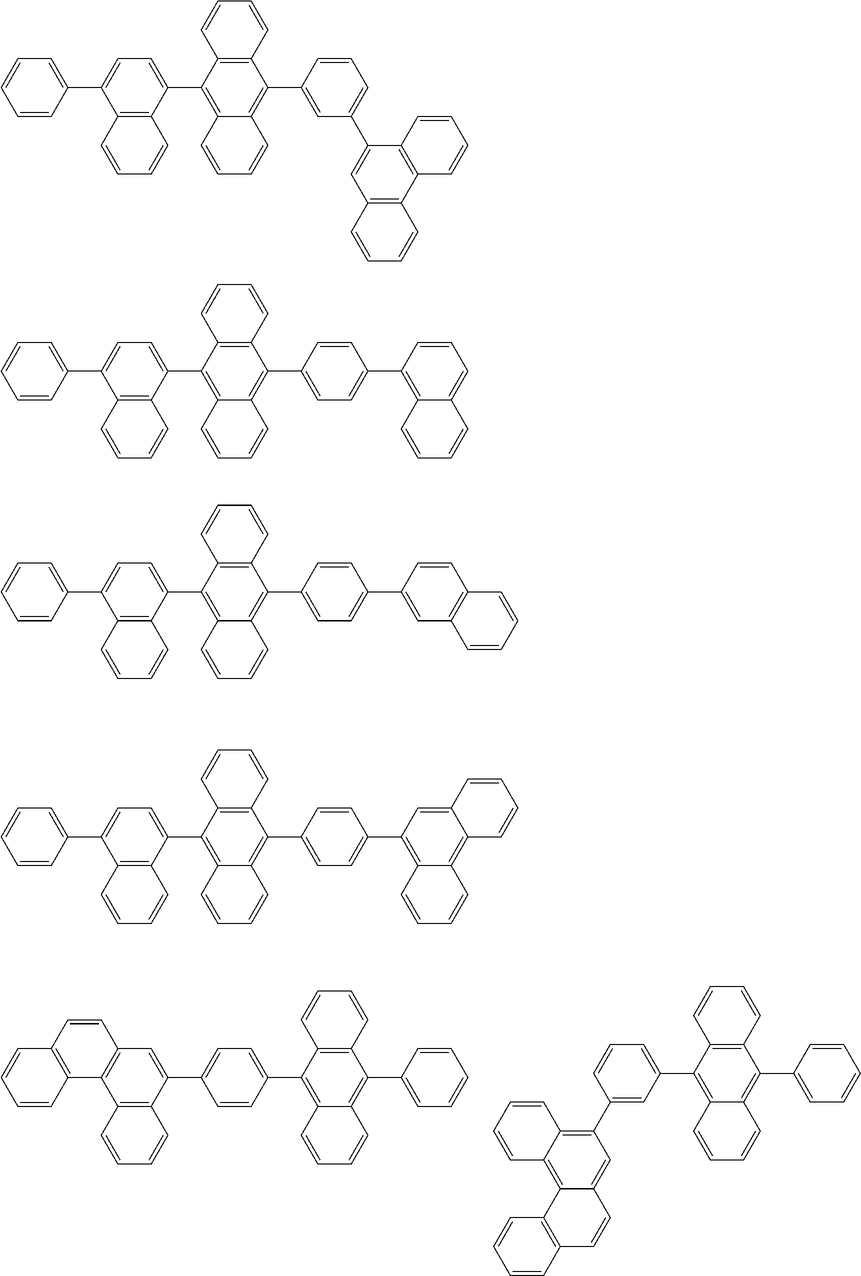

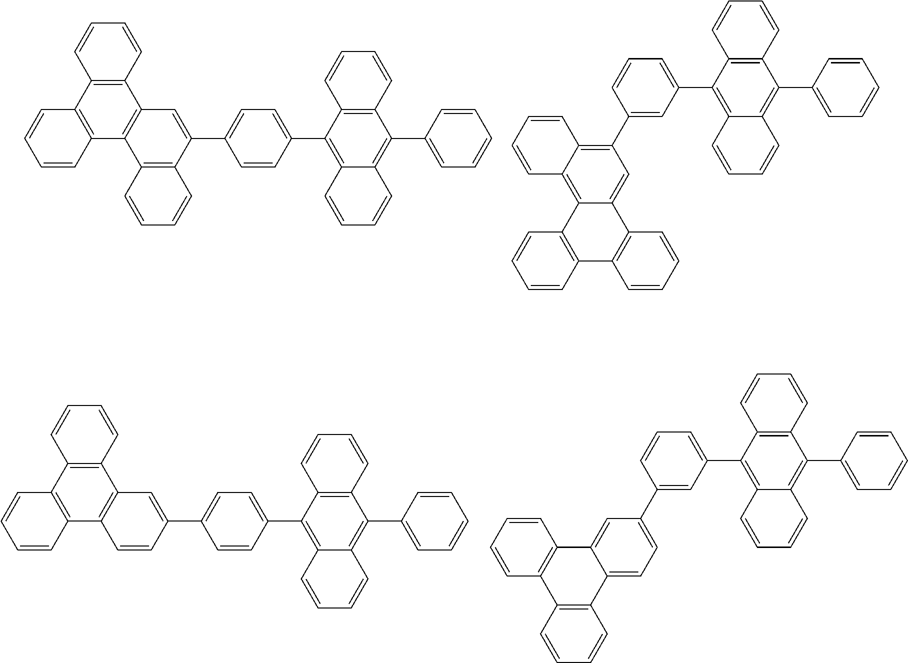

A film thickness of the light emitting zone in the organic EL element is preferably 5 nm or more and 50 nm or less, more preferably 7 nm or more and 50 nm or less, and even more preferably 10 nm or more and 50 nm or less. When the film thickness of the light emitting zone is 5 nm or more, the light emitting zone is easily formed, and a color is easily adjusted. When the film thickness of the light emitting zone is 50 nm or less, an increase in driving voltage is easily prevented.

When the light emitting zone in the organic EL element 1 contains the second compound, a content thereof is preferably 10 mass % or more, more preferably 20 mass % or more, and even more preferably 30 mass % or more. The present embodiment does not exclude the case where the light emitting zone contains a material other than the second compound.

### (Electron Transport Zone)

The electron transport zone includes an electron injection layer, an electron transport layer, a hole blocking layer, and the like. Any layer in the electron transport zone, particularly the electron transport layer, preferably contains one or more selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an alkali metal-containing organic complex, an alkaline earth metal-containing organic complex, and a rare earth metal-containing organic complex.

### (Electron Transport Layer)

The electron transport layer is a layer containing a material having a high electron transporting property (an electron transporting material). As the electron transporting material used for the electron transport layer, use can be made of
(1) a metal complex such as an aluminum complex, a beryllium complex, and a zinc complex,
(2) a heteroaromatic compound such as an imidazole derivative, a benzimidazole derivative, an azine derivative, a carbazole derivative, and a phenanthroline derivative, and
(3) a polymer compound.

Examples of the metal complex include tris(8-quinolinolato)aluminum (III) (abbreviation: Alq), tris(4-methyl-8-quinolinolato)aluminum (abbreviation: Almq3), bis(10-hydroxybenzo[h]quinolinato)beryllium (abbreviation: BeBq2), bis(2-methyl-8-quinolinolato)(4-phenylphenolato)aluminum (III) (abbreviation: BAlq), bis(8-quinolinolato)zinc (II) (abbreviation: Znq), bis[2-(2-benzoxazolyl)phenolato]zinc (II) (abbreviation: ZnPBO), bis[2-(2-benzothiazolyl)phenolato]zinc (II) (abbreviation: ZnBTZ), and (8-quinolinolato)lithium (abbreviation: Liq).

Examples of the heteroaromatic compound include 2-(4-biphenylyl)-5-(4-tert-butylphenyl)-1,3,4-oxadiazole (abbreviation: PBD), 1,3-bis[5-(ptert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazol-2-yl)stilbene (abbreviation: BzOs).

Examples of the polymer compound include poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy).

The above material has an electron mobility of 10⁻⁶ cm²/Vs or more. A material other than the above may be used for the electron transport layer as long as the material has an electron transporting property higher than a hole transporting property.

The electron transport layer may be a single layer or a multi-layer including two or more layers. For example, the electron transport layer may be a layer including a first electron transport layer (on the anode side) and a second electron transport layer (on the cathode side). The two or more electron transport layers are each formed of the above electron transporting material.

### (Electron Injection Layer)

The electron injection layer is a layer containing a material having a high electron injecting property. For the electron injection layer, an alkali metal, an alkaline earth metal, and a compound thereof, such as lithium (Li), cesium (Cs), calcium (Ca), lithium fluoride (LiF), cesium fluoride (CsF), calcium fluoride (CaF₂), and a lithium oxide (LiO) can be used. In addition, a material having an electron transporting property and containing an alkali metal, an alkaline earth metal, or a compound thereof, specifically Alq containing magnesium (Mg) may be used. In this case, electrons can be injected from the cathode more efficiently.

Alternatively a composite material obtained by mixing an organic compound and an electron donor may be used for the electron injection layer. In such a composite material, since the organic compound receives electrons from the electron donor, the electron injecting property and the electron transporting property are excellent. In this case, the organic compound is preferably a material excellent in transporting the received electrons, and specifically for example, a material (a metal complex, a heteroaromatic compound, etc.) constituting the above electron transport layer can be used. The electron donor may be any material as long as it exhibits an electron donating property with respect to the organic compound. Specifically an alkali metal, an alkaline earth metal, and a rare earth metal are preferred, and examples thereof include lithium, cesium, magnesium, calcium, erbium, and ytterbium. In addition, an alkali metal oxide and an alkaline earth metal oxide are preferred, and examples thereof include lithium oxide, calcium oxide, and barium oxide. A Lewis base such as magnesium oxide can also be used. An organic compound such as tetrathiafulvalene (abbreviation: TTF) can also be used.

### (Cathode)

For the cathode, a metal, an alloy an electrically conductive compound, and a mixture thereof, which have a small work function (specifically 3.8 eV or less), are preferably used. Specific examples of such a cathode material include an element belonging to Group 1 or Group 2 in the periodic table, that is, an alkali metal such as lithium (Li) and cesium (Cs), an alkaline earth metal such as magnesium (Mg), calcium (Ca), and strontium (Sr), an alloy containing the same (for example, MgAg or AlLi), a rare earth metal such as europium (Eu) and ytterbium (Yb), or an alloy containing the same.

When forming the cathode using an alkali metal, an alkaline earth metal, or an alloy containing the same, a vacuum deposition method or a sputtering method can be used. When using a silver paste or the like, a coating method, an ink jet method, or the like can be used.

By providing the electron injection layer, the cathode can be formed using various conductive materials such as A1, Ag, ITO, graphene, and silicon or silicon oxide-containing indium tin oxide regardless of the magnitude of the work function. The conductive materials can be formed by using a sputtering method, an ink jet method, a spin coating method, or the like.

### (Insulating Layer)

In the organic EL element, since an electric field is applied to an ultra-thin film, a pixel defect caused by leakage or short circuit is likely to occur. In order to prevent this, an insulating layer made of an insulating thin film layer may be inserted between the pair of electrodes.

Examples of a material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate thereof may be used.

### (Space Layer)

The space layer is, for example, a layer provided between a fluorescent light emitting layer and a phosphorescent light emitting layer for the purpose of not diffusing excitons generated in the phosphorescent light emitting layer into the fluorescent light emitting layer or adjusting carrier balance when the fluorescent light emitting layer and the phosphorescent light emitting layer are laminated. The space layer may be provided between a plurality of phosphorescent light emitting layers. Here, the “carrier” means a charge carrier in a substance.

In order to provide the space layer between the light emitting layers, it is preferred to use a material having both an electron transporting property and a hole transporting property. In addition, in order to prevent diffusion of triplet energy in the adjacent phosphorescent light emitting layer, the triplet energy is preferably 2.6 eV or more. Examples of a material used for the space layer include a material same as those used for the above hole transport layer.

### (Blocking Layer)

A blocking layer such as an electron blocking layer, a hole blocking layer, or an exciton blocking layer may be provided adjacent to the light emitting layer. The electron blocking layer is a layer that prevents electrons from leaking from the light emitting layer to the hole transport layer, and the hole blocking layer is a layer that prevents holes from leaking from the light emitting layer to the electron transport layer. The exciton blocking layer has a function of preventing excitons generated in the light emitting layer from diffusing into peripheral layers and confining the excitons in the light emitting layer.

A film thickness of each layer described above is not particularly limited. Generally when the film thickness is too small, a defect such as a pinhole is likely to occur, and conversely when the film thickness is too large, a high driving voltage is required and efficiency is likely to deteriorate, and therefore, the film thickness is generally 5 nm to 10 μm, and more preferably 10 nm to 0.2 μm. A thickness of the light emitting zone is as described above.

### (Layer Forming Method)

A method of forming each layer of the organic EL element according to the embodiment of the present invention is not particularly limited, and for example, a known method such as a dry film-forming method such as a vacuum deposition method, a molecular beam epitaxy method (an MBE method), a sputtering method, a plasma method, or an ion plating method, or a wet film-forming method such as a spin coating method, a dipping method, a flow coating method, a bar coating method, a roll coating method, or an ink jet method can be adopted.

### [Electronic Device]

An organic EL element according to one embodiment of the present invention can be used in an electronic device such as a display device or a light emitting device. Examples of the display device include a television, a mobile phone, a tablet, a personal computer, and a display component such as an organic EL panel module. Examples of the light emitting device include an illumination and a vehicle lamp.

The organic EL element can be used for the electronic device such as a display device such as a television, a mobile phone, a personal computer, and a display component such as an organic EL panel module, and a light emitting device such as an illumination and a vehicle lamp.

### EXAMPLES

Hereinafter, the present invention will be described in more detail using Examples, but the present invention is not limited to the following Examples.

A compound represented by the formula (1) and used in production of organic EL elements in Examples 1 and 2 and Comparative Examples 2 and 5 to be described later is shown below.

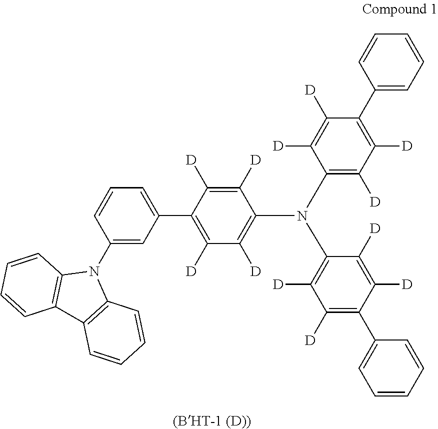 [SMILES: [2H]c1c([2H])c(N(c2c([2H])c([2H])c(-c3ccccc3)c([2H])c2[2H])c2c([2H])c([2H])c(-c3cccc(-n4c5ccccc5c5ccccc54)c3)c([2H])c2[2H])c([2H])c([2H])c1-c1ccccc1]

Compounds represented by the formula (2) and used in production of organic EL elements in Examples 1 and 2 and Comparative Examples 3 and 6 to be described later are shown below.

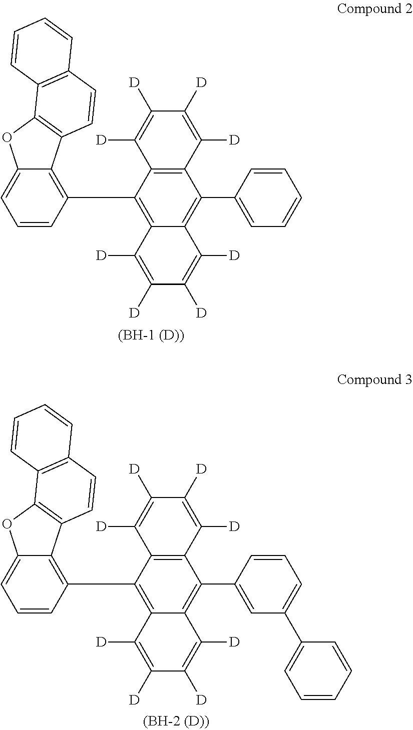 [SMILES: [2H]c1c([2H])c([2H])c2c(-c3cccc4oc5c6ccccc6ccc5c34)c3c([2H])c([2H])c([2H])c([2H])c3c(-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H]]

A compound represented by the formula (1) and used in production of organic EL elements in Examples 3, 4, and 7 and Comparative Examples 8, 11, and 20 to be described later is shown below.

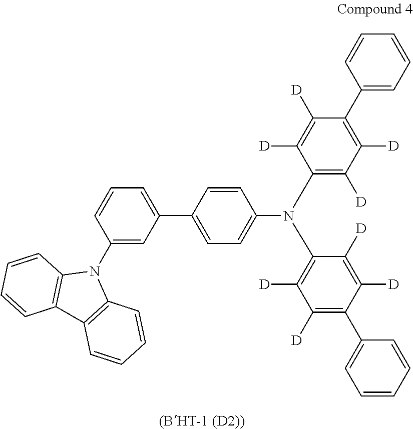 [SMILES: [2H]c1c([2H])c(N(c2ccc(-c3cccc(-n4c5ccccc5c5ccccc54)c3)cc2)c2c([2H])c([2H])c(-c3ccccc3)c([2H])c2[2H])c([2H])c([2H])c1-c1ccccc1]

Compounds represented by the formula (2) and used in production of organic EL elements in Examples 3 and 4 and Comparative Examples 9 and 12 to be described later are shown below.

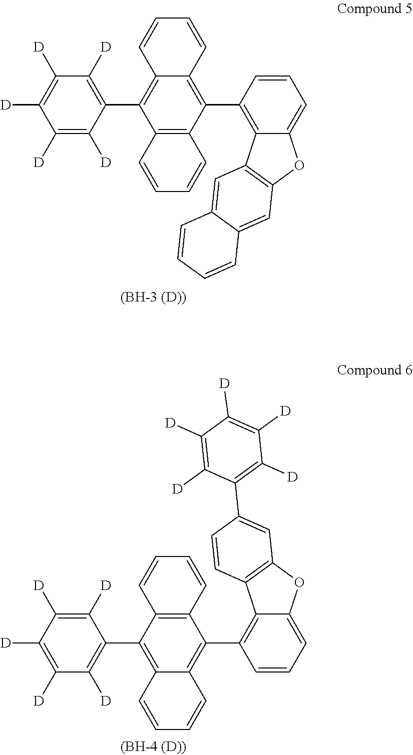 [SMILES: [2H]c1c([2H])c([2H])c(-c2c3ccccc3c(-c3cccc4oc5cc6ccccc6cc5c34)c3ccccc23)c([2H])c1[2H] ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

A compound represented by the formula (1) and used in production of organic EL elements in Examples 5 and 6 and Comparative Examples 14 and 17 to be described later is shown below.

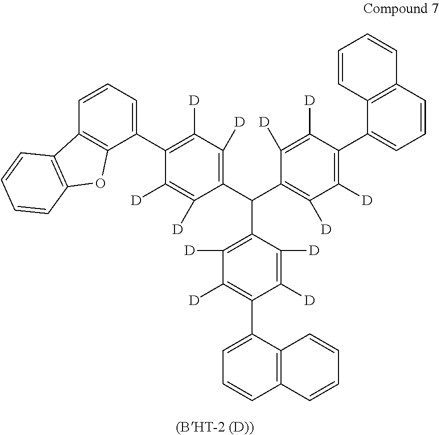 [SMILES: [2H]c1c([2H])c(C(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1cccc2ccccc12]

Compounds represented by the formula (2) and used in production of organic EL elements in Examples 5 and 6 and Comparative Examples 15 and 18 to be described later are shown below.

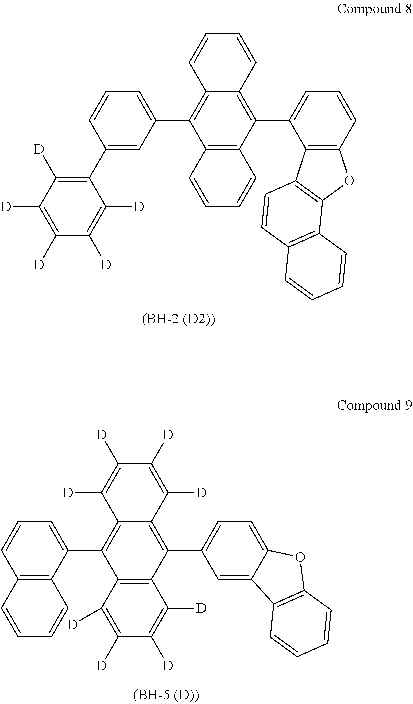 [SMILES: [2H]c1c([2H])c([2H])c(-c2cccc(-c3c4ccccc4c(-c4cccc5oc6c7ccccc7ccc6c45)c4ccccc34)c2)c([2H])c1[2H] ; [2H]c1c([2H])c([2H])c2c(-c3cccc4ccccc34)c3c([2H])c([2H])c([2H])c([2H])c3c(-c3ccc4oc5ccccc5c4c3)c2c1[2H]]

A compound represented by the formula (2) and used in production of organic EL elements in Example 7 and Comparative Example 21 to be described later is shown below.

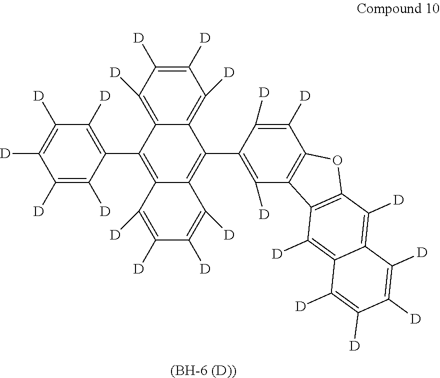 [SMILES: [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; "(BH-6 (D))"]

Compounds used in production of organic EL elements in Comparative Examples 1 to 21 are shown below.

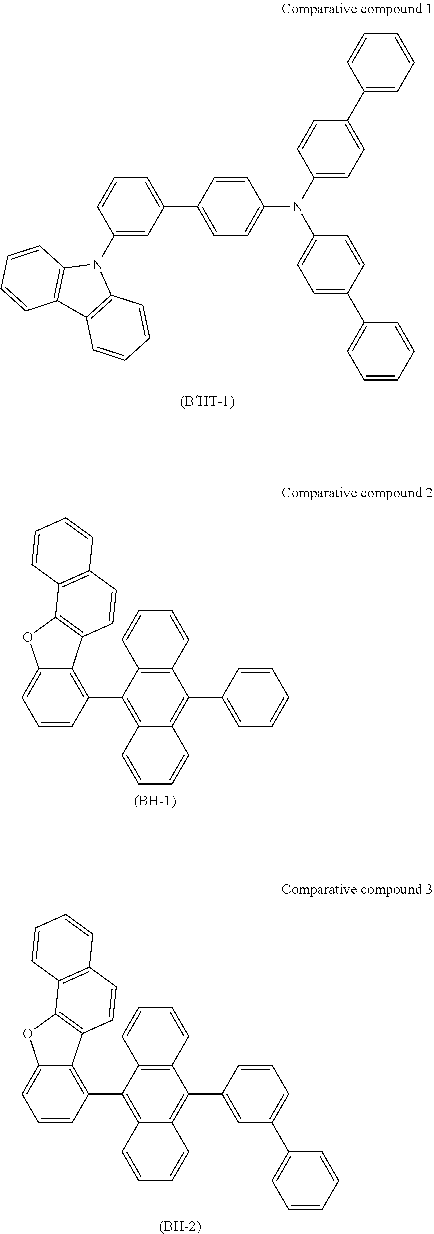

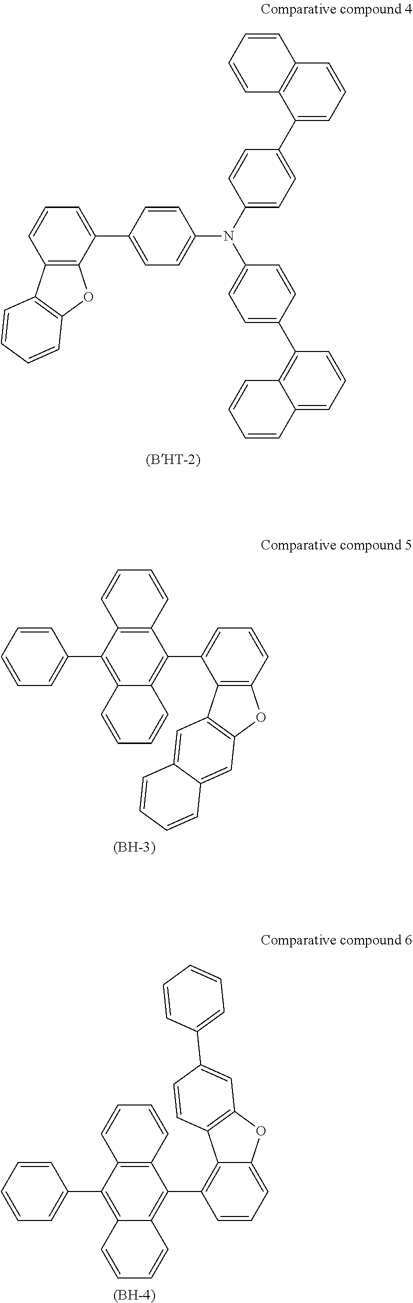

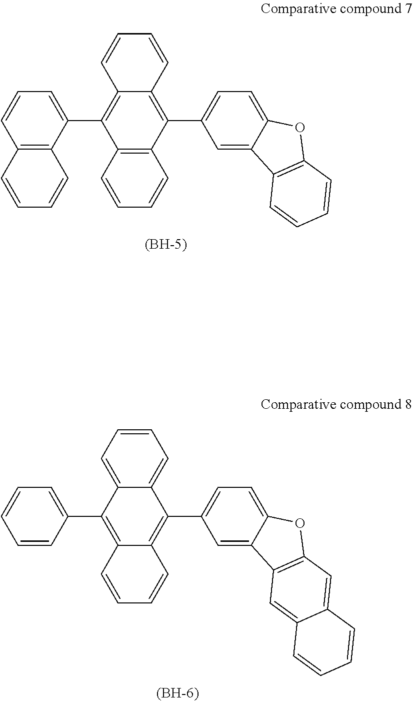

Other compounds used in the production of the organic EL elements in Examples 1 to 7 and Comparative Examples 1 to 21 are shown below.

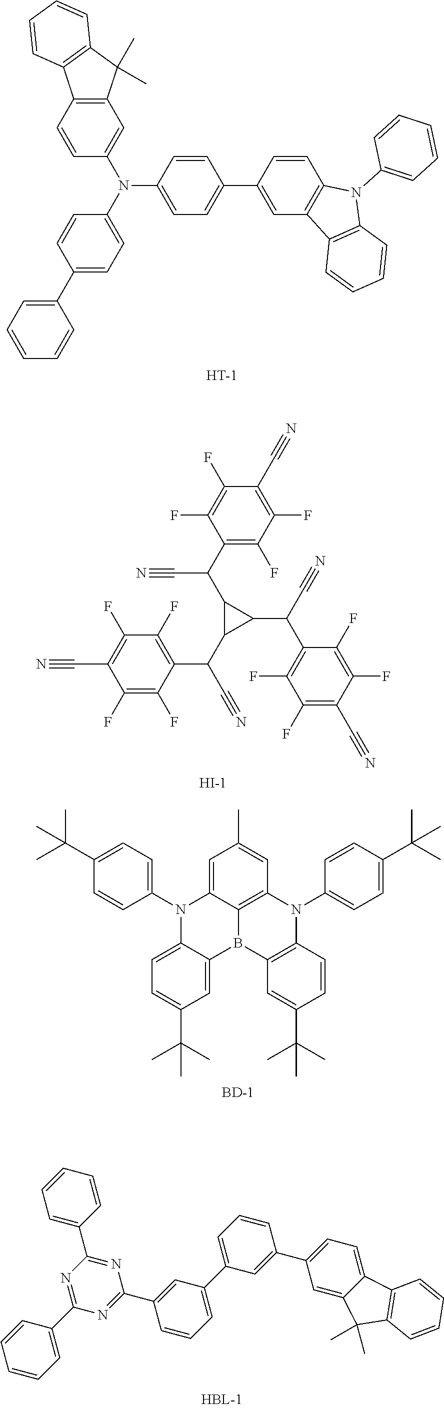

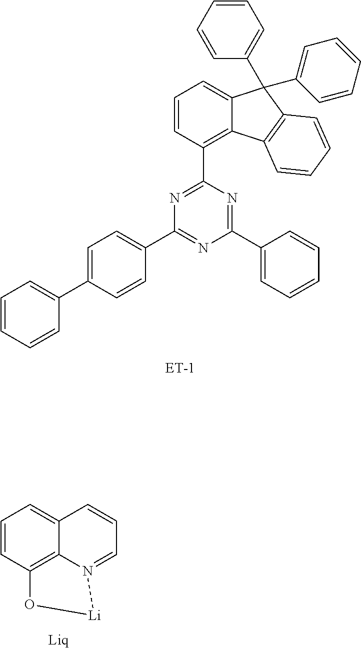

### <Production of Organic EL Element>

An organic EL element was produced as follows.

### Example 1

A 25 mm×75 mm×1.1 mm ITO transparent electrode-attached glass substrate (manufactured by GEOMATEC Co., Ltd.) was subjected to ultrasonic washing in isopropyl alcohol for 5 minutes and then subjected to UV ozone washing for 30 minutes. A thickness of the ITO transparent electrode was 130 nm.

After washing, the ITO transparent electrode-attached glass substrate was mounted on a substrate holder of a vacuum deposition device, and first, the compound HT-1 and the compound HI-1 were co-vapor-deposited so as to cover the ITO transparent electrode, thereby forming a hole injection layer having a film thickness of 10 nm. A concentration of the compound HI-1 in the hole injection layer was 3.0 mass %.

Next, the compound HT-1 (a first hole transport layer material) was vapor-deposited on the hole injection layer to form a first hole transport layer having a film thickness of 85 nm.

Next, the compound 1 (BHT-1(D), a second hole transport layer material) synthesized in Synthesis Example 1 below was vapor-deposited on the first hole transport layer to form a second hole transport layer having a film thickness of 5 nm.

Next, the compound 2 (BH-1(D), a host material) synthesized in Synthesis Example 2 below and the compound BD-1 (a dopant material) were co-vapor-deposited on the second hole transport layer to form a light emitting layer having a film thickness of 20 nm. A concentration of the compound BD-1 in the light emitting layer was 2.0 mass %.

Next, the following compound HBL-1 was vapor-deposited on the light emitting layer to form a hole blocking layer having a film thickness of 5 nm.

Next, the compound ET-1 (an electron transport layer material) and the compound Liq were co-vapor-deposited on the hole blocking layer to form an electron transport layer having a film thickness of 25 nm. A concentration of the compounds ET-1 and Liq in the electron transport layer was 50.0 mass %.

Next, metal A1 was vapor-deposited on the electron transport layer to form a metal A1 cathode having a film thickness of 80 nm.

An element configuration in Example 1 is schematically shown as follows.

ITO (130)/HT-1:HI-1 (10:3%)/HT-1 (85)/B′HT-1(D) (5)/BH-1(D):BD-1 (20:2%)/HBL-1 (5)/ET-1:Liq (25:50%)/A1 (80) The numbers in parentheses indicate a film thickness (unit: nm). The numbers in percentages in the same parentheses indicate ratios (mass %) of the compounds, shown on the right, in the layer. The following Examples 2 to 7 and Comparative Examples 1 to 21 have an element configuration same as that in Example 1 except that at least one of B′HT-1(D) and BH-1(D) used in Example 1 is different, and thus an outline of the element configuration is omitted for these Examples and Comparative Examples.

### Example 2

An organic EL element was produced in the same manner as in Example 1 except that the compound 3 (BH-2(D)) synthesized in Synthesis Example 3 below was used instead of the compound 2 used as the host material for the light emitting layer in Example 1, and the organic EL element was used as an organic EL element in Example 2.

### Comparative Example 1

An organic EL element was produced in the same manner as in Example 1 except that the comparative compound 1 (B′HT-1) was used instead of the compound 1 (B′HT-1(D)) used in the second hole transport layer in Example 1 and the comparative compound 2 (BH-1) was used instead of the compound 2 (BH-1(D)) used as the host material for the light emitting layer in Example 1, and the organic EL element was used as an organic EL element in Comparative Example 1.

### Comparative Example 2

An organic EL element was produced in the same manner as in Example 1 except that the comparative compound 2 (BH-1) was used instead of the compound 2 (BH-1(D)) used as the host material for the light emitting layer in Example 1, and the organic EL element was used as an organic EL element in Comparative Example 2.

### Comparative Example 3

An organic EL element was produced in the same manner as in Example 1 except that the comparative compound 1 (B′HT-1) was used instead of the compound 1 (B′HT-1(D)) used in the second hole transport layer in Example 1, and the organic EL element was used as an organic EL element in Comparative Example 1.

### Comparative Example 4

An organic EL element was produced in the same manner as in Example 2 except that the comparative compound 1 (B′HT-1) was used instead of the compound 1 (B′HT-1(D)) used in the second hole transport layer in Example 2 and the comparative compound 3 (BH-2) was used instead of the compound 3 (BH-2(D)) used as the host material for the light emitting layer in Example 2, and the organic EL element was used as an organic EL element in Comparative Example 4.

### Comparative Example 5

An organic EL element was produced in the same manner as in Example 2 except that the comparative compound 3 (BH-2) was used instead of the compound 3 (BH-2(D)) used as the host material for the light emitting layer in Example 2, and the organic EL element was used as an organic EL element in Comparative Example 5.

### Comparative Example 6

An organic EL element was produced in the same manner as in Example 2 except that the comparative compound 1 (B′HT-1) was used instead of the compound 1 (B′HT-1(D)) used in the second hole transport layer in Example 2, and the organic EL element was used as an organic EL element in Comparative Example 6.

### Example 3

An organic EL element was produced in the same manner as in Example 1 except that the compound 4 (B′HT-1(D2)) was used instead of the compound 1 (B′HT-1(D)) used in the second hole transport layer in Example 1 and the compound 5 (BH-3(D)) was used instead of the compound 2 (BH-1(D)) used as the host material for the light emitting layer in Example 1, and the organic EL element was used as an organic EL element in Example 3.

### Comparative Example 7

An organic EL element was produced in the same manner as in Example 3 except that the comparative compound 1 (B′HT-1) was used instead of the compound 4 used in the second hole transport layer in Example 3 and the comparative compound 5 (BH-3) was used instead of the compound 5 used as the host material for the light emitting layer in Example 3, and the organic EL element was used as an organic EL element in Comparative Example 7.

### Comparative Example 8

An organic EL element was produced in the same manner as in Example 3 except that the comparative compound 5 (BH-3) was used instead of the compound 5 used as the host material for the light emitting layer in Example 3, and the organic EL element was used as an organic EL element in Comparative Example 8.

### Comparative Example 9

An organic EL element was produced in the same manner as in Example 3 except that the comparative compound 1 (B′HT-1) was used instead of the compound 4 used in the second hole transport layer in Example 3, and the organic EL element was used as an organic EL element in Comparative Example 9.

### Example 4

An organic EL element was produced in the same manner as in Example 1 except that the compound 4 (B′HT-1(D2)) was used instead of the compound 1 (B′HT-1(D)) used in the second hole transport layer in Example 1 and the compound 6 (BH-4(D)) was used instead of the compound 2 (BH-1(D)) used as the host material for the light emitting layer in Example 1, and the organic EL element was used as an organic EL element in Example 4.

### Comparative Example 10

An organic EL element was produced in the same manner as in Example 4 except that the comparative compound 1 (B′HT-1) was used instead of the compound 4 used in the second hole transport layer in Example 4 and the comparative compound 6 (BH-4) was used instead of the compound 6 used as the host material for the light emitting layer in Example 4, and the organic EL element was used as an organic EL element in Comparative Example 10.

### Comparative Example 11

An organic EL element was produced in the same manner as in Example 4 except that the comparative compound 6 (BH-4) was used instead of the compound 6 used as the host material for the light emitting layer in Example 4, and the organic EL element was used as an organic EL element in Comparative Example 11.

### Comparative Example 12

An organic EL element was produced in the same manner as in Example 4 except that the comparative compound 1 (B′HT-1) was used instead of the compound 4 used in the second hole transport layer in Example 4, and the organic EL element was used as an organic EL element in Comparative Example 12.

### Example 5

An organic EL element was produced in the same manner as in Example 1 except that the compound 7 (B′HT-2(D)) was used instead of the compound 1 (B′HT-1(D)) used in the second hole transport layer in Example 1 and the compound 8 (BH-2(D2)) was used instead of the compound 2 (BH-1(D)) used as the host material for the light emitting layer in Example 1, and the organic EL element was used as an organic EL element in Example 5.

### Comparative Example 13

An organic EL element was produced in the same manner as in Example 5 except that the comparative compound 4 (B′HT-2) was used instead of the compound 7 used in the second hole transport layer in Example 5 and the comparative compound 7 (BH-5) was used instead of the compound 8 used as the host material for the light emitting layer in Example 5, and the organic EL element was used as an organic EL element in Comparative Example 13.

### Comparative Example 14

An organic EL element was produced in the same manner as in Example 5 except that the comparative compound 7 (BH-5) was used instead of the compound 8 used as the host material for the light emitting layer in Example 5, and the organic EL element was used as an organic EL element in Comparative Example 14.

### Comparative Example 15

An organic EL element was produced in the same manner as in Example 5 except that the comparative compound 4 (B′HT-2) was used instead of the compound 7 used in the second hole transport layer in Example 5, and the organic EL element was used as an organic EL element in Comparative Example 15.

### Example 6

An organic EL element was produced in the same manner as in Example 1 except that the compound 7 (B′HT-2(D)) was used instead of the compound 1 (B′HT-1(D)) used in the second hole transport layer in Example 1 and the compound 9 (BH-5(D)) was used instead of the compound 2 (BH-1(D)) used as the host material for the light emitting layer in Example 1, and the organic EL element was used as an organic EL element in Example 6.

### Comparative Example 16

An organic EL element was produced in the same manner as in Example 6 except that the comparative compound 4 (B′HT-2) was used instead of the compound 7 used in the second hole transport layer in Example 6 and the comparative compound 7 (BH-5) was used instead of the compound 9 used as the host material for the light emitting layer in Example 6, and the organic EL element was used as an organic EL element in Comparative Example 16.

### Comparative Example 17

An organic EL element was produced in the same manner as in Example 6 except that the comparative compound 7 (BH-5) was used instead of the compound 9 used as the host material for the light emitting layer in Example 6, and the organic EL element was used as an organic EL element in Comparative Example 17.

### Comparative Example 18

An organic EL element was produced in the same manner as in Example 6 except that the comparative compound 4 (B′HT-2) was used instead of the compound 7 used in the second hole transport layer in Example 6, and the organic EL element was used as an organic EL element in Comparative Example 18.

### Example 7

An organic EL element was produced in the same manner as in Example 1 except that the compound 4 (B′HT-1(D2)) was used instead of the compound 1 (B′HT-1(D)) used in the second hole transport layer in Example 1 and the compound 10 (BH-6(D)) was used instead of the compound 2 (BH-1(D)) used as the host material for the light emitting layer in Example 1, and the organic EL element was used as an organic EL element in Example 7.

### Comparative Example 19

An organic EL element was produced in the same manner as in Example 7 except that the comparative compound 1 (B′HT-1) was used instead of the compound 4 used in the second hole transport layer in Example 7 and the comparative compound 8 (BH-6) was used instead of the compound 10 used as the host material for the light emitting layer in Example 7, and the organic EL element was used as an organic EL element in Comparative Example 19.

### Comparative Example 20

An organic EL element was produced in the same manner as in Example 7 except that the comparative compound 8 (BH-6) was used instead of the compound 10 used as the host material for the light emitting layer in Example 7, and the organic EL element was used as an organic EL element in Comparative Example 20.

### Comparative Example 21

An organic EL element was produced in the same manner as in Example 7 except that the comparative compound 1 (B′HT-1) was used instead of the compound 4 used in the second hole transport layer in Example 7, and the organic EL element was used as an organic EL element in Comparative Example 21.

### <Evaluation on Organic EL Element>

For the produced organic EL element, a voltage was applied to the organic EL element such that a current density was 50 mA/cm², and a 95% lifetime (LT95) was evaluated. Here, the 95% lifetime (LT95) refers to a time (hr) until the luminance decreases to 95% of the initial luminance during constant current driving.

The results are shown in Tables 1 to 7.

TABLE 1

| | Second hole | Light emitting layer | LT95 |
|---|---|---|---|
| | transport layer | (host material) | [h] |
| Example 1 | B′HT-1(D) | BH-1(D) | 121 |
| Comparative Example 1 | B′HT-1 | BH-1 | 79 |
| Comparative Example 2 | B′HT-1(D) | BH-1 | 86 |
| Comparative Example 3 | B′HT-1 | BH-1(D) | 108 |

TABLE 2

| | Second hole | Light emitting layer | LT95 |
|---|---|---|---|
| | transport layer | (host material) | [h] |
| Example 2 | B′HT-1(D) | BH-2(D) | 96 |
| Comparative Example 4 | B′HT-1 | BH-2 | 58 |
| Comparative Example 5 | B′HT-1(D) | BH-2 | 73 |
| Comparative Example 6 | B′HT-1 | BH-2(D) | 79 |

TABLE 3

| | Second hole | Light emitting layer | LT95 |
|---|---|---|---|
| | transport layer | (host material) | [h] |
| Example 3 | B′HT-1(D2) | BH-3(D) | 101 |
| Comparative Example 7 | B′HT-1 | BH-3 | 75 |
| Comparative Example 8 | B′HT-1(D2) | BH-3 | 79 |
| Comparative Example 9 | B′HT-1 | BH-3(D) | 92 |

TABLE 4

| | Second hole | Light emitting layer | LT95 |
|---|---|---|---|
| | transport layer | (host material) | [h] |
| Example 4 | B′HT-1(D2) | BH-4(D) | 92 |
| Comparative Example 10 | B′HT-1 | BH-4 | 59 |
| Comparative Example 11 | B′HT-1(D2) | BH-4 | 63 |
| Comparative Example 12 | B′HT-1 | BH-4(D) | 80 |

TABLE 5

| | Second hole | Light emitting layer | LT95 |
|---|---|---|---|
| | transport layer | (host material) | [h] |
| Example 5 | B′HT-2(D) | BH-2(D2) | 66 |
| Comparative Example 13 | B′HT-2 | BH-2 | 45 |
| Comparative Example 14 | B′HT-2(D) | BH-2 | 51 |
| Comparative Example 15 | B′HT-2 | BH-2(D2) | 54 |

TABLE 6

| | Second hole | Light emitting layer | LT95 |
|---|---|---|---|
| | transport layer | (host material) | [h] |
| Example 6 | B′HT-2(D) | BH-5(D) | 60 |
| Comparative Example 16 | B′HT-2 | BH-5 | 35 |
| Comparative Example 17 | B′HT-2(D) | BH-5 | 38 |
| Comparative Example 18 | B′HT-2 | BH-5(D) | 47 |

TABLE 7

| | Second hole | Light emitting layer | LT95 |
|---|---|---|---|
| | transport layer | (host material) | [h] |
| Example 7 | B′HT-1(D2) | BH-6(D) | 82 |
| Comparative Example 19 | B′HT-1 | BH-6 | 50 |
| Comparative Example 20 | B′HT-1(D2) | BH-6 | 54 |
| Comparative Example 21 | B′HT-1 | BH-6(D) | 73 |

As is clear from Table 1, it can be seen that when the organic EL element contains the compound B′HT-1(D) encompassed in the formula (1) having a specific structure in the second hole transport layer and the compound BH-1(D) encompassed in the formula (2) having a specific structure, the organic EL element has an LT95 value significantly larger than that of the organic EL elements in Comparative Examples 1 to 3 using either the compound B′HT-1 not encompassed in the formula (1) or the compound BH-1 not encompassed in the formula (2).

As is clear from Table 2, it can be seen that when the organic EL element contains the compound B′HT-1(D) encompassed in the formula (1) having a specific structure in the second hole transport layer and the compound BH-2(D) encompassed in the formula (2) having a specific structure, the organic EL element has an LT95 value significantly larger than that of the organic EL elements in Comparative Examples 4 to 6 using either the compound B′HT-1 not encompassed in the formula (1) or the compound BH-2 not encompassed in the formula (2).

As is clear from Table 3, it can be seen that when the organic EL element contains the compound B′HT-1(D2) encompassed in the formula (1) having a specific structure in the second hole transport layer and the compound BH-3(D) encompassed in the formula (2) having a specific structure, the organic EL element has an LT95 value significantly larger than that of the organic EL elements in Comparative Examples 7 to 9 using either the compound B′HT-1 not encompassed in the formula (1) or the compound BH-3 not encompassed in the formula (2).

As is clear from Table 4, it can be seen that when the organic EL element contains the compound B′HT-1(D2) encompassed in the formula (1) having a specific structure in the second hole transport layer and the compound BH-4(D) encompassed in the formula (2) having a specific structure, the organic EL element has an LT95 value significantly larger than that of the organic EL elements in Comparative Examples 10 to 12 using either the compound B′HT-1 not encompassed in the formula (1) or the compound BH-4 not encompassed in the formula (2).

As is clear from Table 5, it can be seen that when the organic EL element contains the compound B′HT-2(D) encompassed in the formula (1) having a specific structure in the second hole transport layer and the compound BH-2(D) encompassed in the formula (2) having a specific structure, the organic EL element has an LT95 value significantly larger than that of the organic EL elements in Comparative Examples 13 to 15 using either the compound B′HT-2 not encompassed in the formula (1) or the compound BH-2 not encompassed in the formula (2).

As is clear from Table 6, it can be seen that when the organic EL element contains the compound B′HT-2(D) encompassed in the formula (1) having a specific structure in the second hole transport layer and the compound BH-5(D) encompassed in the formula (2) having a specific structure, the organic EL element has an LT95 value significantly larger than that of the organic EL elements in Comparative Examples 16 to 18 using either the compound B′HT-2 not encompassed in the formula (1) or the compound BH-5 not encompassed in the formula (2).

As is clear from Table 7, it can be seen that when the organic EL element contains the compound B′HT-1(D2) encompassed in the formula (1) having a specific structure in the second hole transport layer and the compound BH-6(D) encompassed in the formula (2) having a specific structure, the organic EL element has an LT95 value significantly larger than that of the organic EL elements in Comparative Examples 19 to 21 using either the compound B′HT-1 not encompassed in the formula (1) or the compound BH-6 not encompassed in the formula (2).

### <Synthesis of Compound>

The compounds 1 to 4, 7, and 8 synthesized in Synthesis Examples 1 to 6 below are shown below.

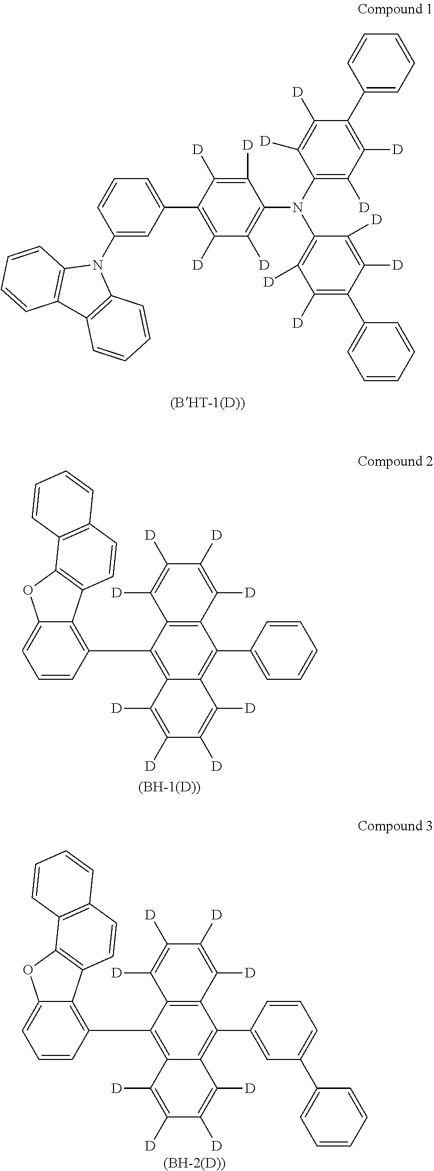

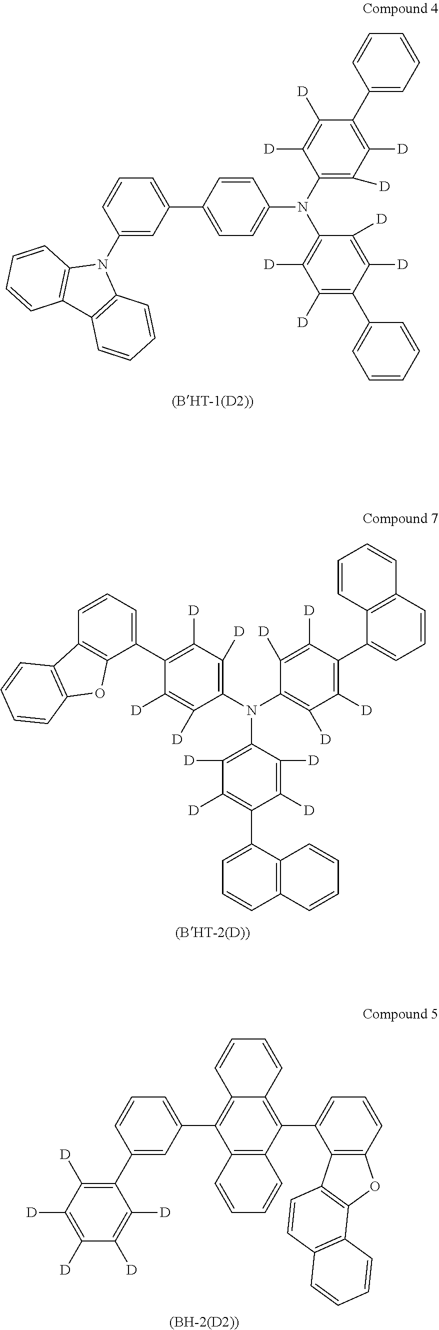 [SMILES: [2H]c1c([2H])c(N(c2ccc(-c3cccc(-n4c5ccccc5c5ccccc54)c3)cc2)c2c([2H])c([2H])c(-c3ccccc3)c([2H])c2[2H])c([2H])c([2H])c1-c1ccccc1 ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1cccc2ccccc12 ; [2H]c1c([2H])c([2H])c(-c2cccc(-c3c4ccccc4c(-c4cccc5oc6c7ccccc7ccc6c45)c4ccccc34)c2)c([2H])c1[2H] ; "(BH-2(D2))"]

### Synthesis Example 1 (Synthesis of Compound 1 (B′HT-1(D))

### Intermediate Synthesis Example 1: Synthesis of Intermediate A

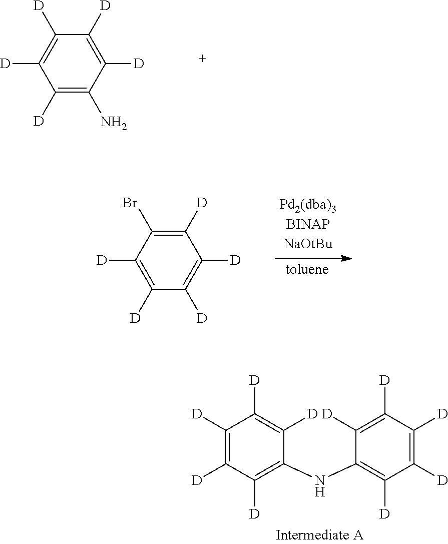 [SMILES: [2H]c1c([2H])c([2H])c(N)c([2H])c1[2H] ; [2H]c1c([2H])c([2H])c(Br)c([2H])c1[2H] ; [2H]c1c([2H])c([2H])c(Nc2c([2H])c([2H])c([2H])c([2H])c2[2H])c([2H])c1[2H]]

Under an argon atmosphere, aniline-2,3,4,5,6-d5 (2.19 g, 22.33 mmol), bromobenzene-d5 (3.29 g, 20.3 mmol), tris(dibenzylideneacetone)dipalladium (0) (372 mg, 0.41 mmol), BINAP (506 mg, 0.812 mmol), sodium-t-butoxide (2.15 g, 22.33 mmol), and toluene (200 m1) were added, followed by heating with stirring at 100° C. for 3 hours. After standing to cool, the residue obtained by filtration was purified by column chromatography to obtain an intermediate A (3.59 g). The yield was 99%.

### Intermediate Synthesis Example 2: Synthesis of Intermediate C

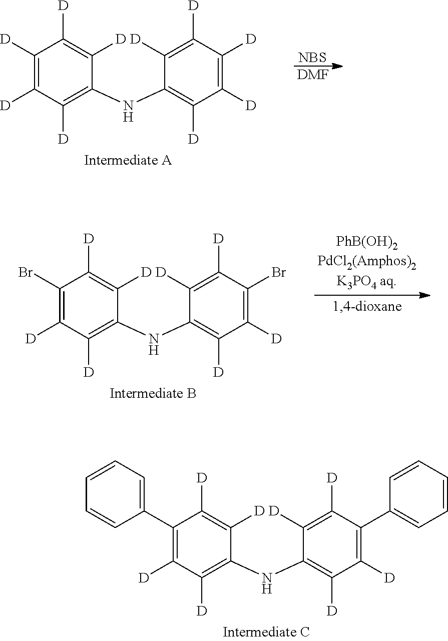

Under an argon atmosphere, the intermediate A (2.9 g, 16.18 mmol) and DMF (55 ml) were mixed, and N-bromosuccinimide (5.76 g, 32.4 mmol) was added thereto at 0° C. An organic layer was extracted by adding water and ethyl acetate and distilled off under reduced pressure to obtain an intermediate B. The intermediate B was subjected to the following reaction without purification.

Under an argon atmosphere, the intermediate B (6.41 g, 19.12 mmol), phenylboronic acid (5.83 g, 47.8 mmol), bis(di-t-butyl(4-dimethylaminophenyl)phosphine)dichloropalladium (II) (406 mg, 0.574 mmol), and 1,4-dioxane (100 m1) were mixed, and a potassium phosphate aqueous solution was added thereto. After heating with stirring at 110° C. for 5 hours and standing to cool, the mixture was filtered, followed by purification by column chromatography and recrystallization to obtain an intermediate C (3.9 g). The yield was 62% (two steps).

### Intermediate Synthesis Example 3: Synthesis of Intermediate 1

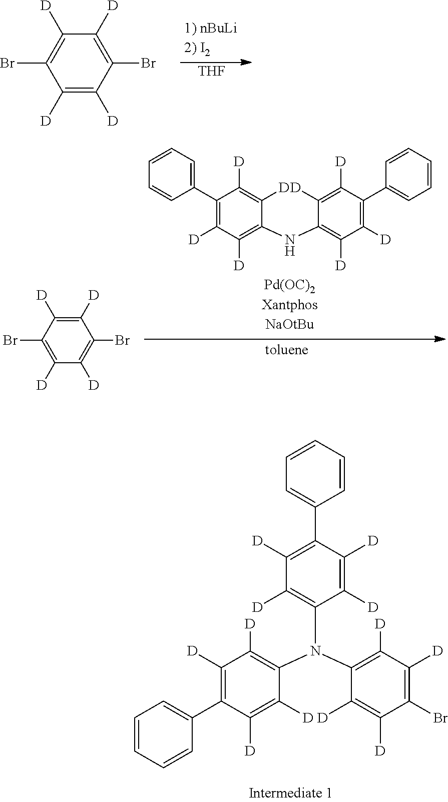 [SMILES: [2H]c1c([2H])c(Br)c([2H])c([2H])c1Br ; [2H]c1c([2H])c(-c2ccccc2)c([2H])c([2H])c1Nc1c([2H])c([2H])c(-c2ccccc2)c([2H])c1[2H] ; [2H]c1c([2H])c(Br)c([2H])c([2H])c1Br ; [2H]c1c([2H])c(N(c2c([2H])c([2H])c(-c3ccccc3)c([2H])c2[2H])c2c([2H])c([2H])c(-c3ccccc3)c([2H])c2[2H])c([2H])c([2H])c1Br ; "THF"]

Under an argon atmosphere, 1,4-dibromobenzene-2,3,5,6-d4 (2.5 g, 10.42 mmol) and THF (105 m1) were mixed, and n-butyllithium (1.59 M, 6.55 m1) was added dropwise at −78° C. Thereafter, a mixture of iodine (3.97 g, 15.63 mmol) and THF (25 m1) was added dropwise at −78° C., followed by stirring for 30 minutes. Thereafter, water and a sodium thiosulfate aqueous solution were added, the temperature was raised to room temperature, and an organic layer was extracted with dichloromethane and distilled off under reduced pressure to obtain 1-bromo-4-iodobenzene-2,3,5,6-d4.

Under an argon atmosphere, the intermediate C (4.12 g, 12.5 mmol), the 1-bromo-4-iodobenzene-2,3,5,6-d4 (4.3 g, 15.0 mmol), palladium(II) acetate (56 mg, 0.25 mmol), 4,5-bis(diphenylphosphino)-9,9-dimethylxanthene (145 mg, 0.25 mmol), sodium-t-butoxide (1.68 g, 17.5 mmol), and toluene (120 m1) were mixed, followed by heating with stirring at 100° C. for 3 hours. After standing to cool, the residue obtained by filtration was suspended and washed with methanol to obtain an intermediate 1. The yield was 98% (two steps).

### Intermediate Synthesis Example 4: Synthesis of Intermediate 2

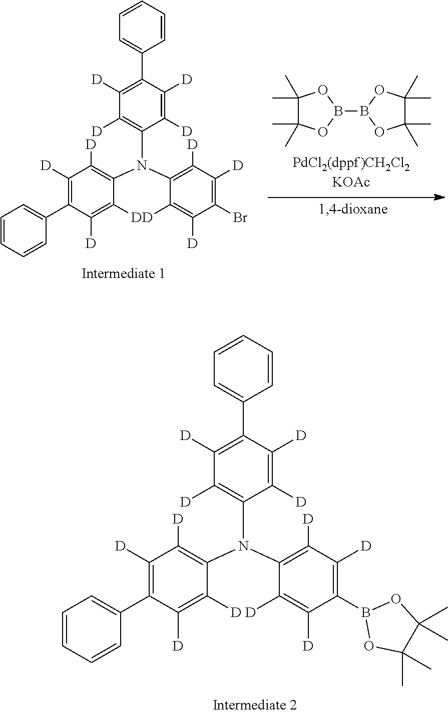

Under an argon atmosphere, the intermediate 1 (4.5 g, 9.21 mmol), bis(pinacolato)diboron (2.81 g, 11.05 mmol), a [1,1′-bis(diphenylphosphino)ferrocene]palladium (II)-dichloride dichloromethane adduct (226 mg, 0.28 mmol), potassium acetate (2.71 g, 27.6 mmol), and 1,4-dioxane (47 m1) were mixed, followed by heating with stirring at 110° C. for 4 hours. After standing to cool, water and ethyl acetate were added for extraction. The obtained organic layer was distilled off under reduced pressure to obtain an intermediate 2.

### Synthesis Example 1: Synthesis of Compound 1 (BHT-1(D))

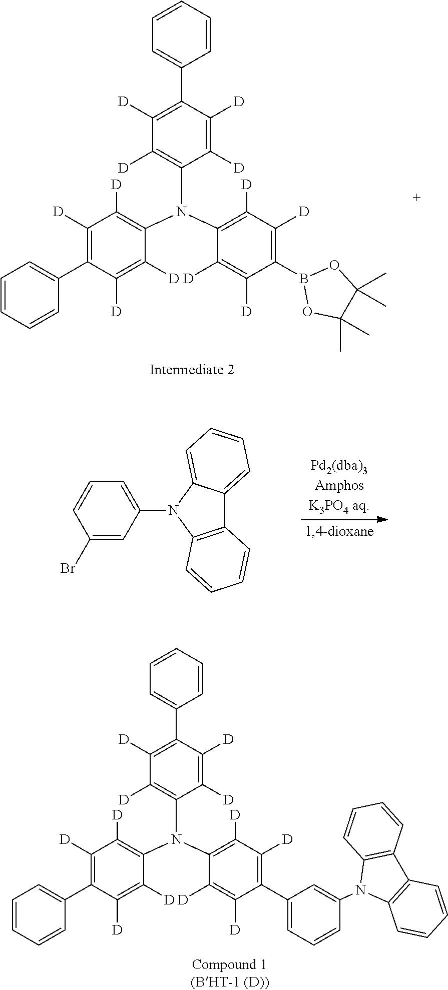 [SMILES: [2H]c1c([2H])c(N(c2c([2H])c([2H])c(-c3ccccc3)c([2H])c2[2H])c2c([2H])c([2H])c(-c3ccccc3)c([2H])c2[2H])c([2H])c([2H])c1B1OC(C)(C)C(C)(C)O1 ; Brc1cccc(-n2c3ccccc3c3ccccc32)c1 ; [2H]c1c([2H])c(N(c2c([2H])c([2H])c(-c3ccccc3)c([2H])c2[2H])c2c([2H])c([2H])c(-c3cccc(-n4c5ccccc5c5ccccc54)c3)c([2H])c2[2H])c([2H])c([2H])c1-c1ccccc1]

Under an argon atmosphere, the intermediate 2 (4.93 g, 9.21 mmol), 9-(3-bromophenyl)carbazole (4.45 g, 13.82 mmol), tris(dibenzylideneacetone)dipalladium (0) (169 mg, 0.18 mmol), [4-(N,N-dimethylamino)phenyl]di-t-butylphosphine (196 mg, 0.74 mmol), potassium phosphate (5.87 g, 27.6 mmol), 1,4-dioxane (75 m1), and water (15 m1) were mixed, followed by heating with stirring at 110° C. for 4 hours. After standing to cool, an organic layer was extracted with toluene and distilled off under reduced pressure. The residue was purified by column chromatography and recrystallization to obtain the compound B′HT-1(D). The yield was 40% (two steps). As a result of mass spectrometry, it was the compound 1 (B′HT-1(D)), and m/e was 650 for a molecular weight of 650.89.

### Synthesis Example 2 (Synthesis of Compound 2 (BH-1(D))

### Intermediate Synthesis Example 5: Synthesis of Intermediate 3

Under an argon atmosphere, 75 ml of toluene, 75 ml of dimethoxyethane, and 75 ml (150.0 mmol) of a 2M Na₂CO₃ aqueous solution were added to 13.3 g (50.0 mmol) of 9-bromoanthracene-d9, 6.4 g (52.5 mmol) of phenylboronic acid, and 1.2 g (1.00 mmol) of Pd[PPh₃]₄, followed by heating under reflux with stirring for 10 hours.

After completion of the reaction, the resultant was cooled to room temperature, and the sample was transferred to a separating funnel and extracted with dichloromethane. An organic layer was dried with MgSO₄, followed by filtration and concentration. The concentration residue was purified by silica gel column chromatography to obtain 10.9 g of a white solid. The obtained compound was subjected to FD-MS analysis, and was identified as the following intermediate 3 (yield: 83%).

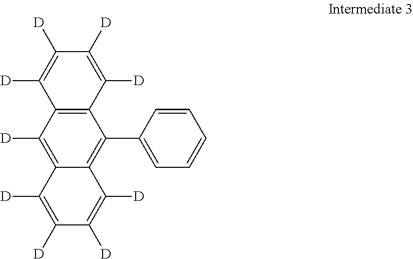 [SMILES: [2H]c1c([2H])c([2H])c2c(-c3ccccc3)c3c([2H])c([2H])c([2H])c([2H])c3c([2H])c2c1[2H]]

### Intermediate Synthesis Example 6: Synthesis of Intermediate 4

To a solution prepared by dissolving 3.2 g (20.0 mmol) of bromine in 12 m1 of dichloromethane was added dropwise at room temperature a solution prepared by dissolving 5.3 g (20.0 mmol) of the intermediate 3 in 120 ml of dichloromethane, followed by stirring for 1 hour.

After completion of the reaction, the sample was transferred to a separating funnel and washed with a 2M Na₂S₂O₃ aqueous solution. Further, an organic phase was washed with 10% Na₂CO₃ and then washed with water, and the separated organic phase was dried with MgSO₄, followed by filtration and concentration.

The concentration residue was dispersed in methanol (100 mL) to precipitate crystals to obtain 6.5 g of a white solid. The obtained compound was subjected to FD-MS analysis, and was identified as the following intermediate 4 (yield: 95%).

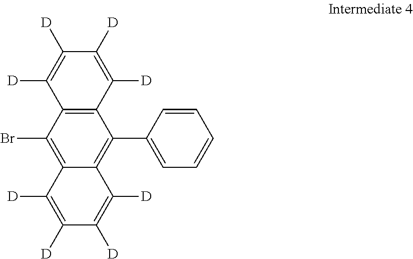 [SMILES: [2H]c1c([2H])c([2H])c2c(-c3ccccc3)c3c([2H])c([2H])c([2H])c([2H])c3c(Br)c2c1[2H]]

### Synthesis Example 2: Synthesis of Compound 2 (BH-1(D))

Under an argon atmosphere, 7.5 ml of toluene, 7.5 ml of dimethoxyethane, and 7.5 ml (15.0 mmol) of a 2M Na₂CO₃ aqueous solution were added to 1.7 g (5.0 mmol) of the intermediate 4, 1.4 g (5.3 mmol) of benzo[b]naphtho[2,1-d]furan-7-boronic acid, and 0.1 g (0.1 mmol) of Pd[PPh₃]₄, followed by heating under reflux with stirring for 10 hours.

After completion of the reaction, the resultant was cooled to room temperature, and the sample was transferred to a separating funnel and extracted with dichloromethane. An organic phase was dried with MgSO₄, followed by filtration and concentration. The concentration residue was purified by silica gel column chromatography to obtain 1.6 g of a white solid. The obtained compound was subjected to FD-MS analysis, and was identified as the following compound BH-1(D) (yield: 75%).

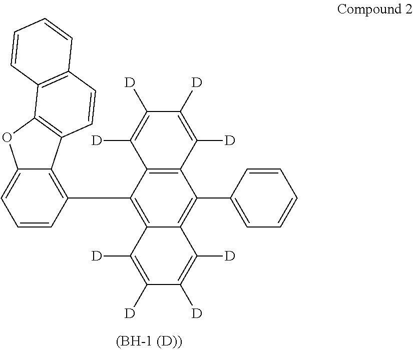 [SMILES: [2H]c1c([2H])c([2H])c2c(-c3cccc4oc5c6ccccc6ccc5c34)c3c([2H])c([2H])c([2H])c([2H])c3c(-c3ccccc3)c2c1[2H]]

### Synthesis Example 3 (Synthesis of Compound 3 (BH-2(D))

### Intermediate Synthesis Example 7: Synthesis of Intermediate 5

Under an argon atmosphere, 75 ml of toluene, 75 ml of dimethoxyethane, and 75 ml (150.0 mmol) of a 2M Na₂CO₃ aqueous solution were added to 13.3 g (50.0 mmol) of 9-bromoanthracene-d9, 10.4 g (52.5 mmol) of 3-biphenylboronic acid, and 1.2 g (1.00 mmol) of Pd[PPh₃]₄, followed by heating under reflux with stirring for 10 hours.

After completion of the reaction, the resultant was cooled to room temperature, and the sample was transferred to a separating funnel and extracted with dichloromethane. An organic phase was dried with MgSO₄, followed by filtration and concentration. The concentration residue was purified by silica gel column chromatography to obtain 13.6 g of a white solid. The obtained compound was subjected to FD-MS analysis, and was identified as the following intermediate 5 (yield: 80%).

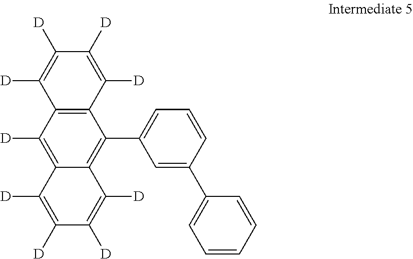 [SMILES: [2H]c1c([2H])c([2H])c2c(-c3cccc(-c4ccccc4)c3)c3c([2H])c([2H])c([2H])c([2H])c3c([2H])c2c1[2H]]

### Intermediate Synthesis Example 8: Synthesis of Intermediate 6

To a solution prepared by dissolving 3.2 g (20.0 mmol) of bromine in 12 m1 of dichloromethane was added dropwise at room temperature a solution prepared by dissolving 6.8 g (20.0 mmol) of the intermediate 6 in 120 ml of dichloromethane, followed by stirring for 1 hour.

After completion of the reaction, the sample was transferred to a separating funnel and washed with a 2M Na₂S₂O₃ aqueous solution. Further, an organic phase was washed with 10% Na₂CO₃, and then washed three times with water. The organic phase was dried with MgSO₄, followed by filtration and concentration.

The concentration residue was dispersed in methanol (100 mL) to precipitate crystals to obtain 8.0 g of a white solid. The obtained compound was subjected to FD-MS analysis, and was identified as the following intermediate 6 (yield: 96%).

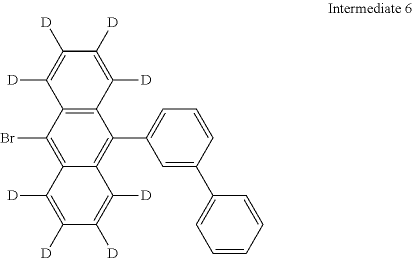 [SMILES: [2H]c1c([2H])c([2H])c2c(-c3cccc(-c4ccccc4)c3)c3c([2H])c([2H])c([2H])c([2H])c3c(Br)c2c1[2H]]

### Synthesis Example 3: Synthesis of Compound 3 (BH-2(D))

Under an argon atmosphere, 7.5 ml of toluene, 7.5 ml of dimethoxyethane, and 7.5 ml (15.0 mmol) of a 2M Na₂CO₃ aqueous solution were added to 2.1 g (5.0 mmol) of the intermediate 6, 1.4 g (5.3 mmol) of benzo[b]naphtho[2,1-d]furan-7-boronic acid, and 0.1 g (0.1 mmol) of Pd[PPh₃]₄, followed by heating under reflux with stirring for 10 hours.

After completion of the reaction, the resultant was cooled to room temperature, and the sample was transferred to a separating funnel and extracted with dichloromethane. An organic phase was dried with MgSO₄, followed by filtration and concentration. The concentration residue was purified by silica gel column chromatography to obtain 1.5 g of a white solid. The obtained compound was subjected to FD-MS analysis, and was identified as the compound 3 (BH-2(D)) (yield: 59%).

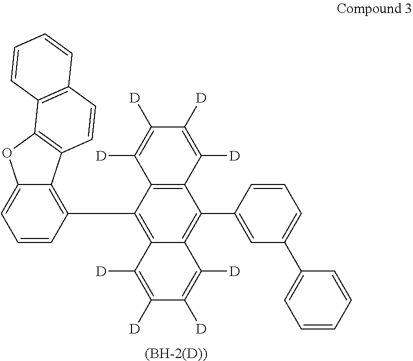

### Synthesis Example 4: Synthesis of Compound 4 (B′HT-1(D2))

The compound B′HT-1(D2) was synthesized by the following synthesis route.

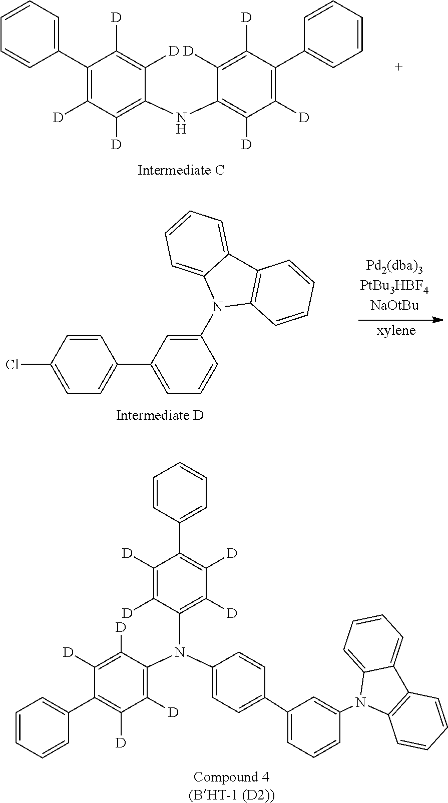 [SMILES: [2H]c1c([2H])c(-c2ccccc2)c([2H])c([2H])c1Nc1c([2H])c([2H])c(-c2ccccc2)c([2H])c1[2H] ; Clc1ccc(-c2cccc(-n3c4ccccc4c4ccccc43)c2)cc1 ; [2H]c1c([2H])c(N(c2ccc(-c3cccc(-n4c5ccccc5c5ccccc54)c3)cc2)c2c([2H])c([2H])c(-c3ccccc3)c([2H])c2[2H])c([2H])c([2H])c1-c1ccccc1]

### Synthesis Example 4: Synthesis of Compound 4 (B′HT-1(D2))

Under an argon atmosphere, the intermediate C (2.9 g, 8.8 mmol), an intermediate D (2.83 g, 8.0 mmol) synthesized by a method known in the literature, tris(dibenzylideneacetone)dipalladium (0) (147 mg, 0.16 mmol), sodium-t-butoxide (1.07 g, 11.2 mmol), and xylene (50 m1) were added, followed by heating with stirring at 140° C. for 1 hour. After standing to cool, the mixture was filtered and purified by column chromatography to synthesize the compound B′HT-1(D2) (3.6 g). The yield was 70%. As a result of mass spectrometry, the obtained compound was the compound B′HT-1(D2), and m/e was 646 for a molecular weight of 646.86.

### Synthesis Example 5 (Synthesis of Compound 7 (B′HT-2(D))

### Intermediate Synthesis Example 9: Synthesis of Intermediate E

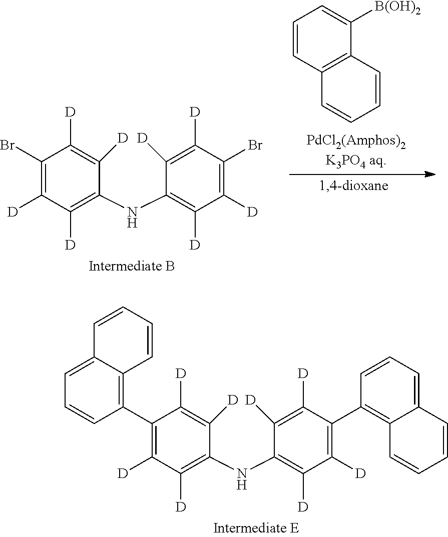 [SMILES: [2H]c1c([2H])c(Nc2c([2H])c([2H])c(Br)c([2H])c2[2H])c([2H])c([2H])c1Br ; OB(O)c1cccc2ccccc12 ; [2H]c1c([2H])c(-c2cccc3ccccc23)c([2H])c([2H])c1Nc1c([2H])c([2H])c(-c2cccc3ccccc23)c([2H])c1[2H]]

Under an argon atmosphere, the intermediate B (6.41 g, 19.12 mmol), 1-naphthylboronic acid (8.22 g, 47.8 mmol), bis(di-t-butyl(4-dimethylaminophenyl)phosphine)dichloropalladium (II) (406 mg, 0.574 mmol), and 1,4-dioxane (100 mL) were mixed, and a potassium phosphate aqueous solution was added thereto. After heating with stirring at 110° C. for 7 hours and standing to cool, the mixture was filtered and purified by column chromatography and recrystallization to obtain an intermediate E (4.9 g). The yield was 71% (two steps).

### Intermediate Synthesis Example 10: Synthesis of Intermediate F

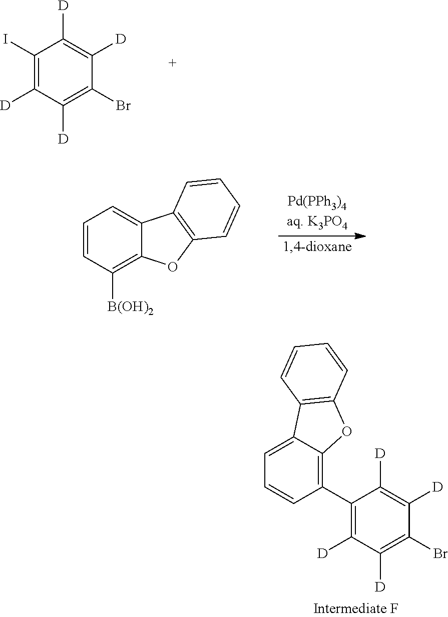 [SMILES: [2H]c1c([2H])c(I)c([2H])c([2H])c1Br ; OB(O)c1cccc2c1oc1ccccc12 ; [2H]c1c([2H])c(-c2cccc3c2oc2ccccc23)c([2H])c([2H])c1Br]

Under an argon atmosphere, a mixture of 25.2 g (52.3 mmol) of 3-bromo-6-iodobenzene-1,2,4,5-d4, 11.1 g (52.4 mmol) of dibenzofuran-4-boronic acid, 1.81 g (1.57 mmol) of tetrakis(triphenylphosphine)palladium (0), 65.4 mL of a 2M potassium phosphate aqueous solution, and 300 mL of 1,4-dioxane was stirred at 80° C. for 8 hours. After the reaction solution was cooled to room temperature, water was added thereto, followed by stirring for 1 hour, and the precipitated solid was collected by filtration. The obtained solid was purified by silica gel column chromatography to obtain 12.4 g of a white solid (an intermediate F). The yield was 73%.

### Synthesis Example 5: Synthesis of Compound 7 (B′HT-2(D))

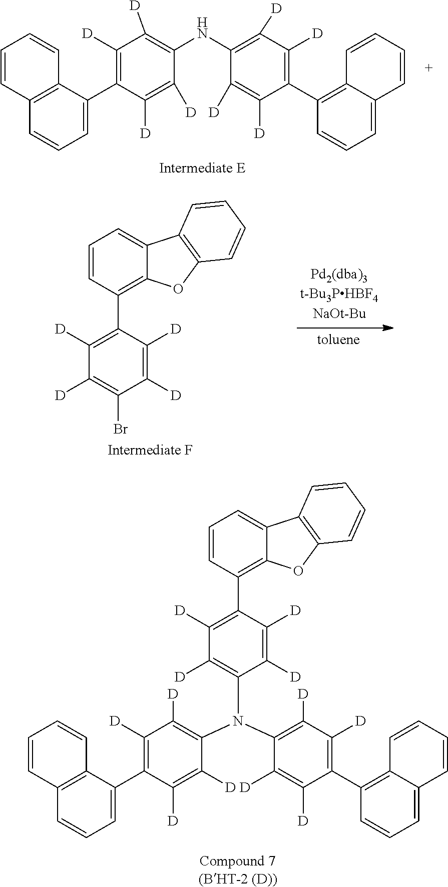 [SMILES: [2H]c1c([2H])c(-c2cccc3ccccc23)c([2H])c([2H])c1Nc1c([2H])c([2H])c(-c2cccc3ccccc23)c([2H])c1[2H] ; [2H]c1c([2H])c(-c2cccc3c2oc2ccccc23)c([2H])c([2H])c1Br ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1cccc2ccccc12]

Under an argon atmosphere, a mixture of 3.01 g (7.00 mmol) of the intermediate E, 2.41 g (7.35 mmol) of the intermediate F, 0.128 g (0.140 mmol) of tris(dibenzylideneacetone)dipalladium (0), 0.162 g (0.560 mmol) of tri-t-butylphosphonium tetrafluoroborate, 0.942 g (9.80 mmol) of sodium-t-butoxide, and 70 mL of toluene was stirred at 100° C. for 6 hours. The reaction solution was cooled to room temperature and then concentrated under reduced pressure. The obtained residue was purified by silica gel column chromatography and recrystallization to obtain 3.90 g of a white solid. The yield was 82%.

As a result of mass spectrometry, the obtained compound was the compound B′HT-2(D), and m/e was 676 for a molecular weight of 675.89.

### Synthesis Example 6: Synthesis of Compound 8 (BH-2(D2))

The compound BH-2(D2) was synthesized by the following synthesis route.

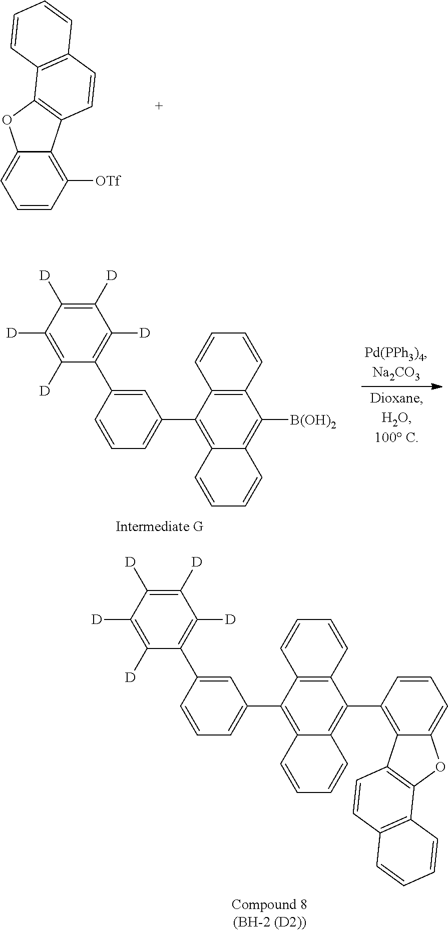 [SMILES: O=S(=O)(Oc1cccc2oc3c4ccccc4ccc3c12)C(F)(F)F ; [2H]c1c([2H])c([2H])c(-c2cccc(-c3c4ccccc4c(B(O)O)c4ccccc34)c2)c([2H])c1[2H] ; [2H]c1c([2H])c([2H])c(-c2cccc(-c3c4ccccc4c(-c4cccc5oc6c7ccccc7ccc6c45)c4ccccc34)c2)c([2H])c1[2H]]

### Synthesis Example 6: Synthesis of Compound 8 (BH-2(D2))

Under an argon atmosphere, 1.93 g of naphtho[1,2-b]benzofuran-7-yl trifluoromethanesulfonate synthesized by a known method, 2.00 g of a boronic acid derivative (an intermediate G) synthesized by a known method, 0.24 g of tetrakis(triphenylphosphine)palladium (0) (Pd(PPh₃)₄), 1.21 g of sodium carbonate, 40 mL of toluene, and 13 mL of ion-exchanged water were added to a flask, followed by stirring under reflux for 18 hours. After cooling to room temperature, the precipitated solid was collected by filtration. The obtained solid was washed with water and acetone, and then recrystallized in a mixed solvent of toluene and hexane to obtain 2.02 g of a pale yellow solid (yield: 69%). As a result of mass spectrometry, the pale yellow solid was the compound BH-2(D2), and m/e was 552 for a molecular weight of 551.70.

The compound BH-3(D), the compound BH-4(D), the compound BH-5(D), and the compound BH-6(D) can be synthesized according to a known synthesis technique, and thus detailed description of the synthesis procedure is omitted.

### REFERENCE SIGNS LIST

1, 11 Organic EL element
2 Substrate
3 Anode
4 Cathode
5 Light emitting zone (light emitting layer)
6 Hole transport zone (hole transport layer)
6a First hole transport layer
6b Second hole transport layer
7 Electron transport zone (electron transport layer)
7a First electron transport layer
7b Second electron transport layer
10, 20 Light emitting unit

1. An organic electroluminescent element comprising:
an anode;
a cathode; and
an organic layer disposed between the anode and the cathode and including a light emitting zone, wherein
the organic layer comprises a first layer containing a first compound and a second layer containing a second compound,
the first layer and the second layer are different layers,
the first compound contains 1 mass % or more of a compound having one or more deuterium atoms in a compound represented by the following formula (1), and
the second compound contains 1 mass % or more of a compound having one or more deuterium atoms in a compound represented by the following formula (2), 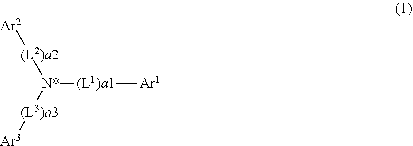
wherein
N* is a central nitrogen atom,
L¹ to L³ each independently represent a single bond, a substituted or unsubstituted arylene group having 6 to 30 ring carbon atoms, or a substituted or unsubstituted divalent heterocyclic group having 5 to 30 ring atoms,
a1 to a3 are each independently an integer of 1 to 3, and
Ar¹ to Ar³ each independently represent a substituted or unsubstituted aryl group having 6 to 30 ring carbon atoms or a substituted or unsubstituted monovalent heterocyclic group having 5 to 30 ring atoms, 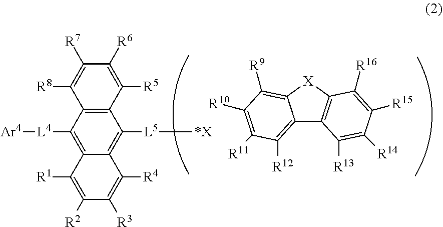
wherein
X represents an oxygen atom, a sulfur atom, or NR¹⁰⁰,
L⁴ and L⁵ each independently represent a single bond, a substituted or unsubstituted arylene group having 6 to 30 ring carbon atoms, or a substituted or unsubstituted divalent heterocyclic group having 5 to 30 ring atoms,
Ar⁴ represents a substituted or unsubstituted aryl group having 6 to 30 ring carbon atoms or a substituted or unsubstituted monovalent heterocyclic group having 5 to 30 ring atoms,
one of R⁹ to R¹⁶ and R¹⁰⁰ is bonded to *x,
R¹ to R⁸, and R⁹ to R¹⁶ and R¹⁰⁰ that are not bonded to *x each independently represent a hydrogen atom or a substituent A,
the substituent A is a substituted or unsubstituted alkyl group having 1 to 50 carbon atoms or a substituted or unsubstituted aryl group having 6 to 50 ring carbon atoms, and
adjacent two selected from R⁹ to R¹⁶ that are not bonded to *x form a ring or do not form a ring.

2. The organic electroluminescent element according to claim 1, wherein the compound represented by the formula (1) is represented by the following formula (1-1a), formula (1-1b), or formula (1-1c), 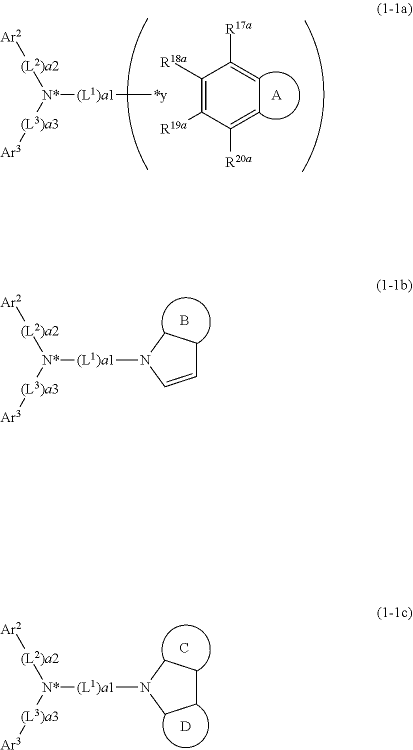
in the formula (1-1a),
a ring A is a substituted or unsubstituted aromatic hydrocarbon ring having 6 to 50 ring carbon atoms, or a substituted or unsubstituted heterocyclic ring having 5 to 50 ring atoms,
one of R^{17a} to R^{20a} is bonded to *y
R^{17a} to R^{20a} that are not bonded to *y each independently represent a hydrogen atom or a substituent B,
the substituent B is a substituted or unsubstituted alkyl group having 1 to 50 carbon atoms or a substituted or unsubstituted aryl group having 6 to 50 ring carbon atoms, and
adjacent two selected from R^{17a} to R^{20a} that are not bonded to *y form a ring or do not form a ring,
in the formula (1-1b),
a ring B is a substituted or unsubstituted aromatic hydrocarbon ring having 6 to 50 ring carbon atoms, or a substituted or unsubstituted heterocyclic ring having 5 to 50 ring atoms,
in the formula (1-1c),
a ring C and a ring D are each independently a substituted or unsubstituted aromatic hydrocarbon ring having 6 to 50 ring carbon atoms, or a substituted or unsubstituted heterocyclic ring having 5 to 50 ring atoms, and
in the formula (1-1a) to the formula (1-1c), N*, L¹ to L³, Ar², Ar³, and a1 to a3 are as defined in the formula (1).

3. The organic electroluminescent element according to claim 1, wherein
the compound represented by the formula (1) is represented by the following formula (1-2), 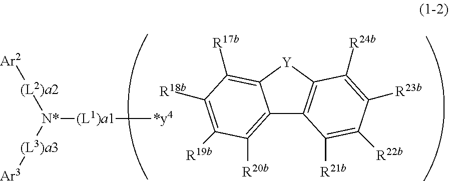
wherein
Y represents an oxygen atom, a sulfur atom, NR¹⁰¹, or CR¹⁰²R¹⁰³,
one of R^{17b} to R^{24b} and R¹⁰¹ is bonded to *y¹,
R¹⁰², R¹⁰³, and R^{17b} to R^{24b} and R¹⁰¹ that are not bonded to *y¹ each independently represent a hydrogen atom or a substituent C,
the substituent C is a substituted or unsubstituted alkyl group having 1 to 50 carbon atoms or a substituted or unsubstituted aryl group having 6 to 50 ring carbon atoms, and
N*, L¹ to L³, Ar², Ar³, and a1 to a3 are as defined in the formula (1).

4. The organic electroluminescent element according to claim 3, wherein
in the formula (1-2), R^{17b} to R^{24b} that are not bonded to L¹ each independently represent a hydrogen atom or an alkyl group having 1 to 10 carbon atoms, and
at least one of R^{17b} to R^{24b} that are not bonded to L¹ represents an alkyl group having 1 to 10 carbon atoms.

5. The organic electroluminescent element according to claim 1, wherein
the compound represented by the formula (1) is represented by the following formula (1-3a) or formula (1-3b), 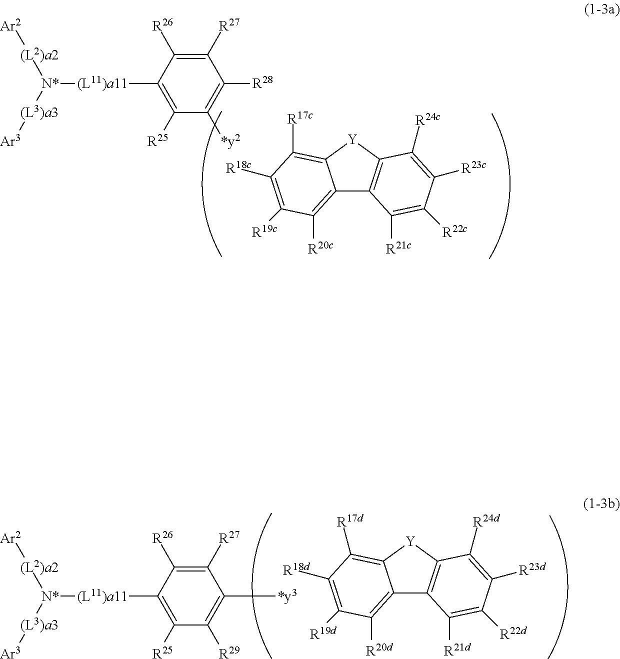
wherein
L¹¹ represents a single bond, a substituted or unsubstituted arylene group having 6 to 30 ring carbon atoms, or a substituted or unsubstituted divalent heterocyclic group having 5 to 30 ring atoms,
a11 is an integer of 1 or 2,
Y represents an oxygen atom, a sulfur atom, NR¹⁰¹, or CR¹⁰²R¹⁰³,
in the formula (1-3a), one of R^{17c} to R^{24c} and R¹⁰¹ is bonded to *y², and R¹⁰², R¹⁰³, and R^{17c} to R^{24c} and R¹⁰¹ that are not bonded to *y² each independently represent a hydrogen atom or a substituent C,
in the formula (1-3b), one of R^{17d} to R^{24d} and R¹⁰¹ is bonded to *y³, and R¹⁰², R¹⁰³, and R^{17d} to R^{24d} and R¹⁰¹ that are not bonded to *y³ each independently represent a hydrogen atom or a substituent C,
the substituent C is a substituted or unsubstituted alkyl group having 1 to 50 carbon atoms or a substituted or unsubstituted aryl group having 6 to 50 ring carbon atoms,
R²⁵ to R²⁸ in the formula (1-3a) and R²⁵ to R²⁷ and R²⁹ in the formula (1-3b) each independently represent a hydrogen atom or a substituent D,
the substituent D is a substituted or unsubstituted alkyl group having 1 to 50 carbon atoms or a substituted or unsubstituted aryl group having 6 to 50 ring carbon atoms,
R²⁶ and R²⁷, R²⁷ and R²⁸ in the formula (1-3a), and R²⁶ and R²⁷, R²⁵ and R²⁹ in the formula (1-3b) each independently form a ring or do not form a ring,
R²⁵ or R²⁶ in the formula (1-3a) and the formula (1-3b) is bonded or is not bonded to a ring carbon atom in L¹¹ adjacent thereto,
N*, L², L³, Ar², Ar³, a2, and a3 are as defined in the formula (1).

6. The organic electroluminescent element according to claim 5, wherein
the compound represented by the formula (1) is represented by the formula (1-3a).

7. The organic electroluminescent element according to claim 1, wherein
in the compound represented by the formula (1), a total number of carbon atoms of groups bonded to the central nitrogen atom is 10 or more.

8. The organic electroluminescent element according to claim 1, wherein
the compound represented by the formula (1) is represented by the following formula (1-4), 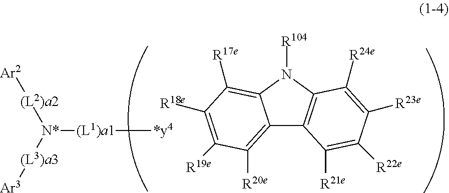
wherein
R¹⁰⁴ represents a hydrogen atom or a substituent E,
the substituent E is a substituted or unsubstituted alkyl group having 1 to 50 carbon atoms or a substituted or unsubstituted aryl group having 6 to 50 ring carbon atoms,
Y represents an oxygen atom, a sulfur atom, NR¹⁰¹, or CR¹⁰²R¹⁰³;
one of R^{17e} to R^{24e} and R¹⁰¹ is bonded to *y⁴,
R¹⁰², R¹⁰³, and R^{17e} to R^{24e} and R¹⁰¹ that are not bonded to *y⁴ each independently represent a hydrogen atom or a substituent C,
the substituent C is a substituted or unsubstituted alkyl group having 1 to 50 carbon atoms or a substituted or unsubstituted aryl group having 6 to 50 ring carbon atoms,
N*, L¹ to L³, Ar², Ar³, and a1 to a3 are as defined in the formula (1).

9. The organic electroluminescent element according to claim 1, wherein
the compound represented by the formula (1) is represented by the following formula (1-5), 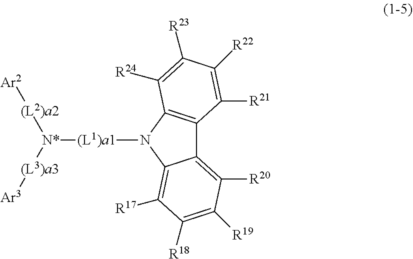
wherein
R¹⁷ to R²⁴ each independently represent a hydrogen atom or a substituent C,
the substituent C is a substituted or unsubstituted alkyl group having 1 to 50 carbon atoms or a substituted or unsubstituted aryl group having 6 to 50 ring carbon atoms,
N*, L¹ to L³, Ar², Ar³, and a1 to a3 are as defined in the formula (1).

10. The organic electroluminescent element according to claim 1, wherein
the compound represented by the formula (1) is represented by the following formula (1-6a) or formula (1-6b), 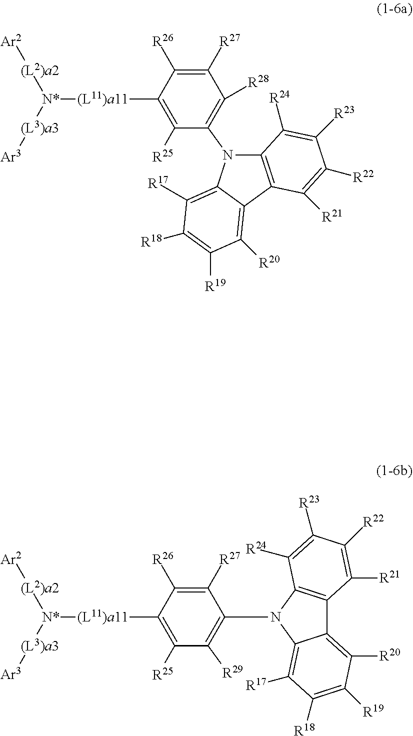
in the formula (1-6a) and the formula (1-6b), N*, L², L³, Ar², Ar³, a2, and a3 are as defined in the formula (1),
R¹⁷ to R²⁴ each independently represent a hydrogen atom or a substituent C,
the substituent C is a substituted or unsubstituted alkyl group having 1 to 50 carbon atoms or a substituted or unsubstituted aryl group having 6 to 50 ring carbon atoms, and
R²⁵ to R²⁸ in the formula (1-6a) and R²⁵ to R²⁷ and R²⁹ in the formula (1-6b) each independently represent a hydrogen atom or a substituent D,
the substituent D is a substituted or unsubstituted alkyl group having 1 to 50 carbon atoms or a substituted or unsubstituted aryl group having 6 to 50 ring carbon atoms,
R²⁶ and R²⁷, R²⁷ and R²⁸ in the formula (1-6a), and R²⁶ and R²⁷, R²⁵ and R²⁹ in the formula (1-6b) each independently form a ring or do not form a ring,
R²⁵ or R²⁶ in the formula (1-6a) and the formula (1-6b) is bonded or is not bonded to a ring carbon atom in L¹¹ adjacent thereto.

11. The organic electroluminescent element according to claim 10, wherein
the compound represented by the formula (1) is represented by the formula (1-6a).

12. The organic electroluminescent element according to claim 1, wherein
the compound represented by the formula (2) is represented by the following formula (2-1), 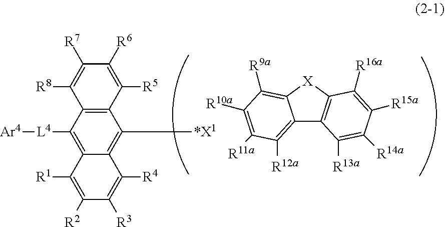
wherein
X represents an oxygen atom, a sulfur atom, or NR¹⁰⁰,
one of R^{9a} to R^{16a} and R¹⁰⁰ is bonded to *x¹,
R^{9a} to R^{16a} and R¹⁰⁰ that are not bonded to *x¹ each independently represent a hydrogen atom or a substituent A, and
L⁴, Ar⁴, X, R¹ to R⁸, and the substituent A are as defined in the formula (2).

13. The organic electroluminescent element according to claim 1, wherein
the compound represented by the formula (2) is represented by any one of the following formula (2-2a) to formula (2-2c), 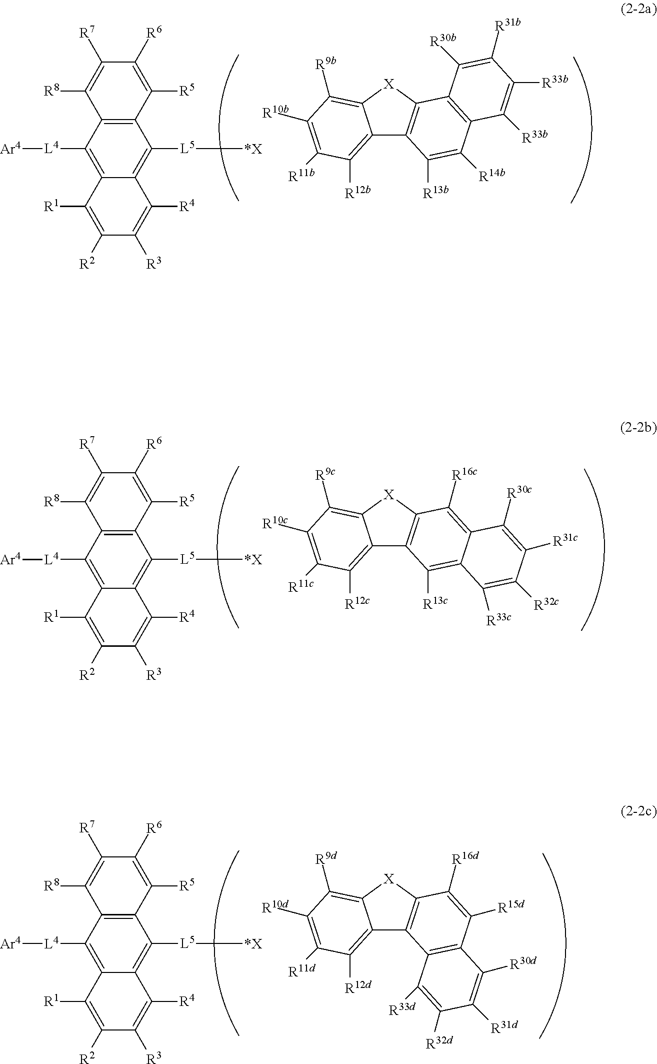
wherein
X represents an oxygen atom, a sulfur atom, or NR¹⁰⁰,
in the formula (2-2a), one of R^{9b} to R^{14b}, R^{30b} to R^{33b}, and R¹⁰⁰ is bonded to *x, and R^{9b} to R^{14b}, R^{30b} to R^{33b}, and R¹⁰⁰ that are not bonded to *x each independently represent a hydrogen atom or a substituent A,
in the formula (2-2b), one of R^{9c} to R^{13c}, R^{16c}, R^{30c} to R^{33c}, and R¹⁰⁰ is bonded to *x, and R^{9c} to R^{13c}, R^{16c}, R^{30c} to R^{33c}, and R¹⁰⁰ that are not bonded to *x each independently represent a hydrogen atom or a substituent A,
in the formula (2-2c), one of R^{9d} to R^{12d}, R^{15d}, R^{16d}, R^{30d} to R^{33d}, and R¹⁰⁰ is bonded to *x, and R^{9d} to R^{12d}, R^{15d}, R^{16d}, R^{30d} to R^{33d}, and R¹⁰⁰ that are not bonded to *x each independently represent a hydrogen atom or a substituent A, and
in the formula (2-2a) to the formula (2-2c), L⁴, L⁵, Ar⁴, and the substituent A are as defined in the formula (2).

14. The organic electroluminescent element according to claim 1, wherein
Ar⁴ represents a substituted or unsubstituted aryl group having 6 to 30 ring carbon atoms.

15. The organic electroluminescent element according to claim 1, wherein
a substituent for the case of “substituted or unsubstituted” is at least one substituent selected from the group consisting of
a cyano group,
a halogen atom,
an alkyl group having 1 to 30 carbon atoms,
a cycloalkyl group having 3 to 30 ring carbon atoms,
an aralkyl group having 7 to 36 carbon atoms,
an alkoxy group having 1 to 30 carbon atoms,
an aryloxy group having 6 to 30 ring carbon atoms,
a tri-substituted silyl group having a substituent selected from an alkyl group having 1 to 30 carbon atoms,
a haloalkyl group having 1 to 30 carbon atoms,
an aryl group having 6 to 30 ring carbon atoms,
an alkylthio group having 1 to 30 carbon atoms, and
an arylthio group having 6 to 30 ring carbon atoms.

16. The organic electroluminescent element according to claim 1, wherein
the first compound includes a plurality of compounds represented by the formula (1) and having structures different from each other, and
the second compound includes a plurality of compounds represented by the formula (2) and having structures different from each other.

17. The organic electroluminescent element according to claim 1, wherein
in the compound represented by the formula (1), the one or more deuterium atom is at least one of a hydrogen atom directly bonded to the arylene group represented by L¹, a hydrogen atom directly bonded to the divalent heterocyclic group represented by L¹, a hydrogen atom directly bonded to the arylene group represented by L², a hydrogen atom directly bonded to the divalent heterocyclic group represented by L², a hydrogen atom directly bonded to the arylene group represented by L³, a hydrogen atom directly bonded to the divalent heterocyclic group represented by L³, a hydrogen atom directly bonded to the aryl group represented by Ar¹, a hydrogen atom directly bonded to the monovalent heterocyclic group represented by Ar¹, a hydrogen atom directly bonded to the aryl group represented by Ar², a hydrogen atom directly bonded to the monovalent heterocyclic group represented by Ar², a hydrogen atom directly bonded to the aryl group represented by Ar³, and a hydrogen atom directly bonded to the monovalent heterocyclic group represented by Ar³.

18. The organic electroluminescent element according to claim 1, wherein
in the compound represented by the formula (1), the one or more deuterium atom is at least one of the hydrogen atom directly bonded to the arylene group represented by L¹, the hydrogen atom directly bonded to the divalent heterocyclic group represented by L¹, the hydrogen atom directly bonded to the arylene group represented by L², the hydrogen atom directly bonded to the divalent heterocyclic group represented by L², the hydrogen atom directly bonded to the arylene group represented by L³, and the hydrogen atom directly bonded to the divalent heterocyclic group represented by L³.

19. The organic electroluminescent element according to claim 1, wherein
the first compound contains 10 mass % or more of, in the compound represented by the formula (1), a compound in which at least one of the hydrogen atom directly bonded to the arylene group represented by L¹, the hydrogen atom directly bonded to the divalent heterocyclic group represented by L¹, the hydrogen atom directly bonded to the arylene group represented by L², the hydrogen atom directly bonded to the divalent heterocyclic group represented by L², the hydrogen atom directly bonded to the arylene group represented by L³, and the hydrogen atom directly bonded to the divalent heterocyclic group represented by L³ is a deuterium atom.

20. The organic electroluminescent element according to claim 1, wherein
the first compound contains the following compounds A1 and A2, and the second compound contains the following compounds B1 and B2 or compounds C1 and C2,

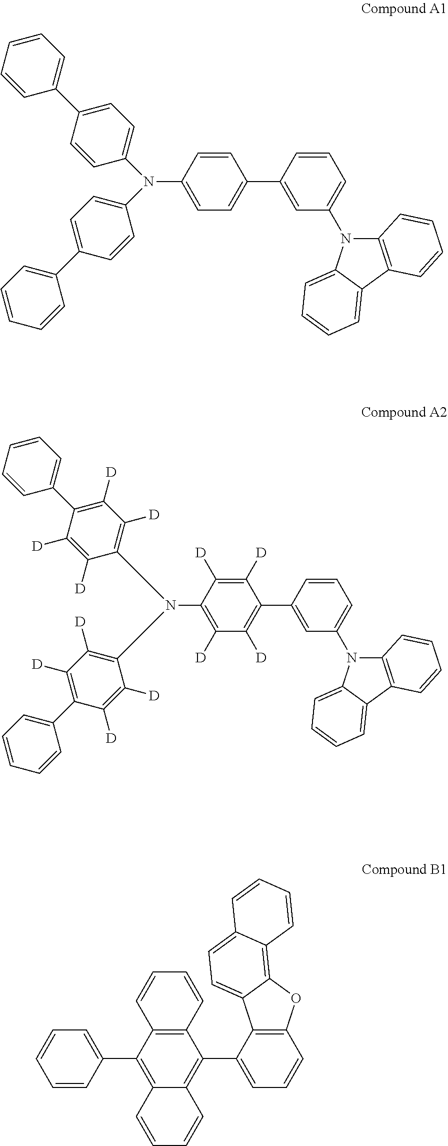 [SMILES: c1ccc(-c2ccc(N(c3ccc(-c4ccccc4)cc3)c3ccc(-c4cccc(-n5c6ccccc6c6ccccc65)c4)cc3)cc2)cc1 ; [2H]c1c([2H])c(N(c2c([2H])c([2H])c(-c3ccccc3)c([2H])c2[2H])c2c([2H])c([2H])c(-c3cccc(-n4c5ccccc5c5ccccc54)c3)c([2H])c2[2H])c([2H])c([2H])c1-c1ccccc1 ; c1ccc(-c2c3ccccc3c(-c3cccc4oc5c6ccccc6ccc5c34)c3ccccc23)cc1]

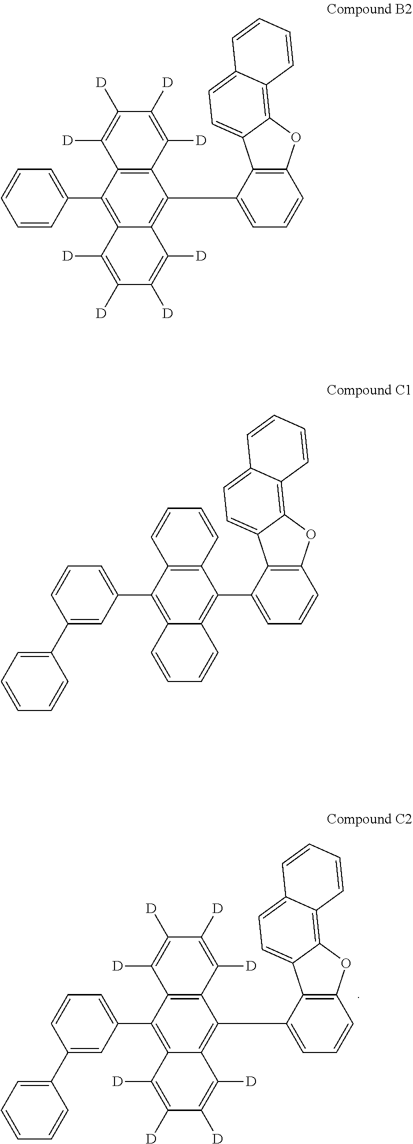 [SMILES: [2H]c1c([2H])c([2H])c2c(-c3cccc4oc5c6ccccc6ccc5c34)c3c([2H])c([2H])c([2H])c([2H])c3c(-c3ccccc3)c2c1[2H] ; c1ccc(-c2cccc(-c3c4ccccc4c(-c4cccc5oc6c7ccccc7ccc6c45)c4ccccc34)c2)cc1 ; [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H]]

21. The organic electroluminescent element according to claim 1, wherein
the first compound contains the following compounds D1 and D2, and the second compound contains the following compounds E1 and E2, compounds F1 and F2, or compounds G1 and G2,

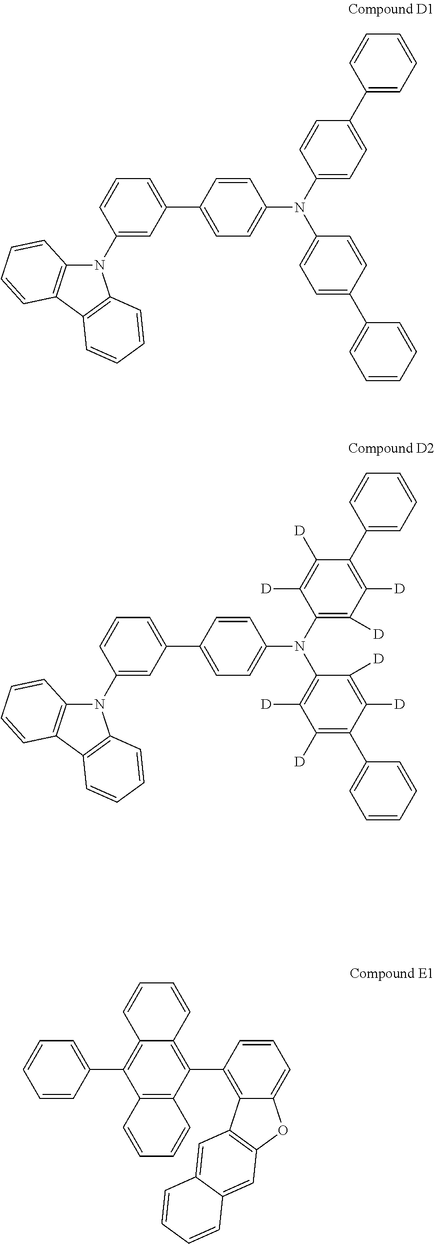 [SMILES: c1ccc(-c2ccc(N(c3ccc(-c4ccccc4)cc3)c3ccc(-c4cccc(-n5c6ccccc6c6ccccc65)c4)cc3)cc2)cc1 ; [2H]c1c([2H])c(N(c2ccc(-c3cccc(-n4c5ccccc5c5ccccc54)c3)cc2)c2c([2H])c([2H])c(-c3ccccc3)c([2H])c2[2H])c([2H])c([2H])c1-c1ccccc1 ; c1ccc(-c2c3ccccc3c(-c3cccc4oc5cc6ccccc6cc5c34)c3ccccc23)cc1]

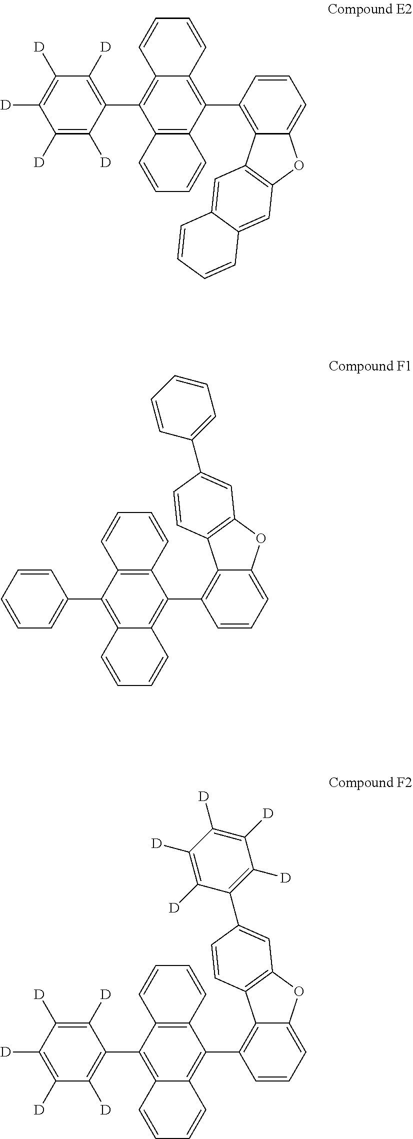 [SMILES: [2H]c1c([2H])c([2H])c(-c2c3ccccc3c(-c3cccc4oc5cc6ccccc6cc5c34)c3ccccc23)c([2H])c1[2H] ; c1ccc(-c2ccc3c(c2)oc2cccc(-c4c5ccccc5c(-c5ccccc5)c5ccccc45)c23)cc1 ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

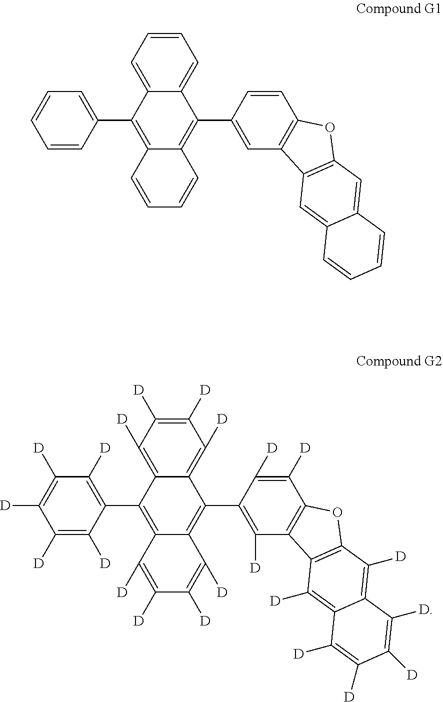

22. The organic electroluminescent element according to claim wherein
the first compound contains the following compounds H1 and H2, and the second compound contains the following compounds I1 and I2 or compounds J1 and J2,

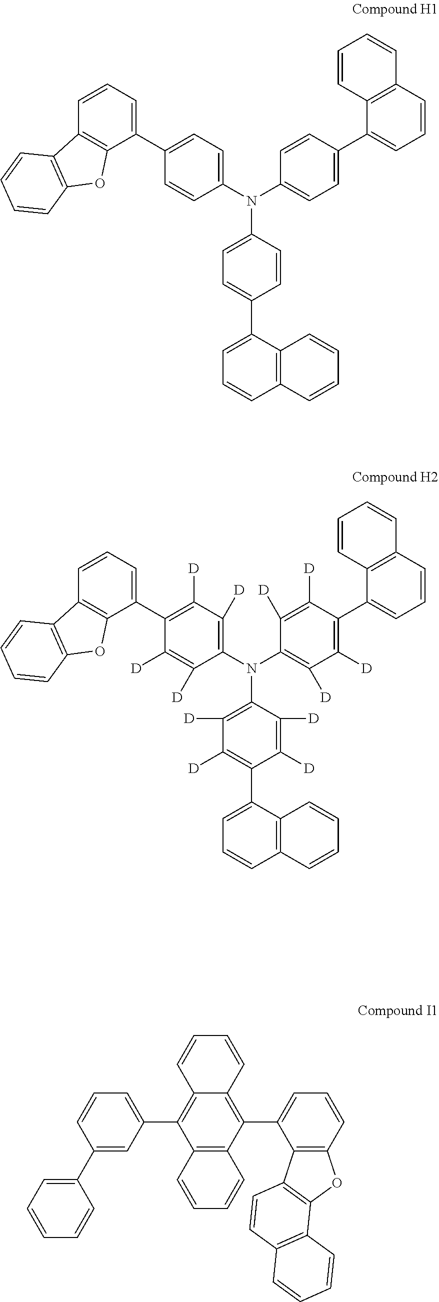 [SMILES: c1ccc2c(-c3ccc(N(c4ccc(-c5cccc6ccccc56)cc4)c4ccc(-c5cccc6c5oc5ccccc56)cc4)cc3)cccc2c1 ; [2H]c1c([2H])c(N(c2c([2H])c([2H])c(-c3cccc4ccccc34)c([2H])c2[2H])c2c([2H])c([2H])c(-c3cccc4c3oc3ccccc34)c([2H])c2[2H])c([2H])c([2H])c1-c1cccc2ccccc12 ; c1ccc(-c2cccc(-c3c4ccccc4c(-c4cccc5oc6c7ccccc7ccc6c45)c4ccccc34)c2)cc1]

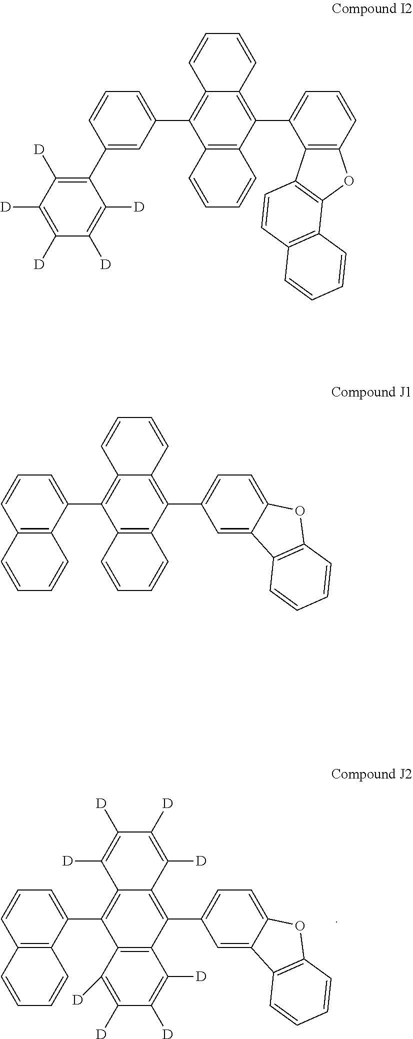 [SMILES: [2H]c1c([2H])c([2H])c(-c2cccc(-c3c4ccccc4c(-c4cccc5oc6c7ccccc7ccc6c45)c4ccccc34)c2)c([2H])c1[2H] ; c1ccc2c(-c3c4ccccc4c(-c4ccc5oc6ccccc6c5c4)c4ccccc34)cccc2c1 ; [2H]c1c([2H])c([2H])c2c(-c3cccc4ccccc34)c3c([2H])c([2H])c([2H])c([2H])c3c(-c3ccc4oc5ccccc5c4c3)c2c1[2H]]

23. The organic electroluminescent element according to claim 1, wherein
the organic layer includes a hole transport zone between the anode and the light emitting zone, and the first layer is included in the hole transport zone.

24. The organic electroluminescent element according to claim 23, wherein
the hole transport zone includes a third layer different from the first layer.

25. The organic electroluminescent element according to claim 24, wherein
the third layer is disposed between the anode and the first layer, and the third layer does not contain the first compound.

26. The organic electroluminescent element according to claim 24, wherein
no other layers are included between the first layer and the light emitting zone.

27. The organic electroluminescent element according to claim 24, wherein
the hole transport zone includes the first layer, and the light emitting zone includes the second layer.

28. The organic electroluminescent element according to claim 1, wherein
the light emitting zone contains a fluorescent dopant material.

29. The organic electroluminescent element according to claim 1, wherein
the light emitting zone contains a phosphorescent dopant material.

30. The organic electroluminescent element according to claim 1, wherein
the light emitting zone contains the second compound.

31. An electronic device comprising:
the organic electroluminescent element according to claim 1.
